SEQUENCE LISTING (420 sequences)

SEQ ID NO: 1 - DNA, 45 bp, synthetic construct
  source 1..45: mol_type = other DNA
agtagaaacaagggtgtttttcctcatatctctgaaattctaatc

SEQ ID NO: 2 - DNA, 34 bp, synthetic construct
  source 1..34: mol_type = other DNA
agtagaaacaaggtcgtttttaaactattcgaca

SEQ ID NO: 3 - DNA, 43 bp, synthetic construct
  source 1..43: mol_type = other DNA
agtagaaacaaggcattttttcatgaaggacaagctaaattca

SEQ ID NO: 4 - DNA, 58 bp, synthetic construct
  source 1..58: mol_type = other DNA
agtagaaacaaggtacttttttggacagtatggatagcaaatagtagcactgccacaa

SEQ ID NO: 5 - DNA, 45 bp, synthetic construct
  source 1..45: mol_type = other DNA
agtagaaacaagggtgtttttcctcatatttctgaaattctaatc

SEQ ID NO: 6 - DNA, 23 bp, synthetic construct
  source 1..23: mol_type = other DNA
agtagaaacaagggtatttttct

SEQ ID NO: 7 - DNA, 28 bp, synthetic construct
  source 1..28: mol_type = other DNA
agtagaaacaaggagttttttgaacaga

SEQ ID NO: 8 - DNA, 20 bp, synthetic construct
  source 1..20: mol_type = other DNA
agtagaaacaaggtagtttt

SEQ ID NO: 9 - DNA, 26 bp, synthetic construct
  source 1..26: mol_type = other DNA
agtagaaacaagggtgttttttatta

SEQ ID NO: 10 - DNA, 94 bp, synthetic construct
  source 1..94: mol_type = other DNA
agtagtaacaagagcatttttcaataacgtttctttgtaatgacaacaagtaaacaagcactacaataaaggaaaatacagggcttaacttccc

SEQ ID NO: 11 - DNA, 89 bp, synthetic construct
  source 1..89: mol_type = other DNA
agtagaaacacgagccttttttcattttaatcatttgtgtatcgcatgtattcaatgatgaccaataaccccataaacatcttcgaagc

SEQ ID NO: 12 - DNA, 60 bp, synthetic construct
  source 1..60: mol_type = other DNA
agtagaaacacgagcatttttcactcaattgcgttcattgaattaatggataaatttata

SEQ ID NO: 13 - DNA, 95 bp, synthetic construct
  source 1..95: mol_type = other DNA
agtagaaacacgtgcatttttgattcttggttctttttattggatgtttagatacataatgaacaaaatagtaccaaattgagtaccatttcctt

SEQ ID NO: 14 - DNA, 101 bp, synthetic construct
  source 1..101: mol_type = other DNA
agtagaaacaacagcattttttatatttattttaataagaataaacacccacattccaaacgtattgaaacaatcacagtcatagtgtctattttgttgcc

SEQ ID NO: 15 - DNA, 103 bp, synthetic construct
  source 1..103: mol_type = other DNA
agtagtaacaagagcatttttcagaaacaattaagtttagtaaggacaattgttcaaacaaaacaggaacaaagggtttagaacagactcaaccattcctcca

SEQ ID NO: 16 - DNA, 89 bp, synthetic construct
  source 1..89: mol_type = other DNA
agtagaaacaacgcactttttccagtttatttgctgacattgattacaatttgcttaaatgcatagtaagaaatacagtaaaattgaat

SEQ ID NO: 17 - DNA, 30 bp, synthetic construct
  source 1..30: mol_type = other DNA
agtagtaacaagaggatttttattttaaat

SEQ ID NO: 18 - DNA, 32 bp, synthetic construct
  source 1..32: mol_type = other DNA
tttggttgtttttattttcccctgcttttgct

SEQ ID NO: 19 - DNA, 32 bp, synthetic construct
  source 1..32: mol_type = other DNA
tttggttgtttttattttcccctgtttctact

SEQ ID NO: 20 - DNA, 27 bp, synthetic construct
  source 1..27: mol_type = other DNA
attgaatataattgacctgctttcgct

SEQ ID NO: 21 - DNA, 27 bp, synthetic construct
  source 1..27: mol_type = other DNA
attgaatataattgacctgtttccact

SEQ ID NO: 22 - DNA, 27 bp, synthetic construct
  source 1..27: mol_type = other DNA
attgaatataattgacctgcttttgct

SEQ ID NO: 23 - DNA, 27 bp, synthetic construct
  source 1..27: mol_type = other DNA
attgaatataattgacctgtttctact

SEQ ID NO: 24 - DNA, 24 bp, synthetic construct
  source 1..24: mol_type = other DNA
tcaaatggtttgcctgctttcgct

SEQ ID NO: 25 - DNA, 24 bp, synthetic construct
  source 1..24: mol_type = other DNA
tcaaatggtttgcctgtttccact

SEQ ID NO: 26 - DNA, 24 bp, synthetic construct
  source 1..24: mol_type = other DNA
tcaaatggtttgcctgcttttgct

SEQ ID NO: 27 - DNA, 24 bp, synthetic construct
  source 1..24: mol_type = other DNA
tcaaatggtttgcctgtttctact

SEQ ID NO: 28 - DNA, 24 bp, synthetic construct
  source 1..24: mol_type = other DNA
tttggatcagtacctgctttcgct

SEQ ID NO: 29 - DNA, 24 bp, synthetic construct
  source 1..24: mol_type = other DNA
tttggatcagtacctgtttccact

SEQ ID NO: 30 - DNA, 24 bp, synthetic construct
  source 1..24: mol_type = other DNA
tttggatcagtacctgcttttgct

SEQ ID NO: 31 - DNA, 24 bp, synthetic construct
  source 1..24: mol_type = other DNA
tttggatcagtacctgtttctact

SEQ ID NO: 32 - DNA, 45 bp, synthetic construct
  source 1..45: mol_type = other DNA
gattttgatgtcactcagtgagtgattatctaccctgcttttgct

SEQ ID NO: 33 - DNA, 45 bp, synthetic construct
  source 1..45: mol_type = other DNA
gattttgatgtcactcagtgagtgattatctaccctgtttctact

SEQ ID NO: 34 - DNA, 20 bp, synthetic construct
  source 1..20: mol_type = other DNA
tttaaacccctgctttcgct

SEQ ID NO: 35 - DNA, 20 bp, synthetic construct
  source 1..20: mol_type = other DNA
tttaaacccctgtttccact

SEQ ID NO: 36 - DNA, 20 bp, synthetic construct
  source 1..20: mol_type = other DNA
tttaaacccctgcttttgct

SEQ ID NO: 37 - DNA, 20 bp, synthetic construct
  source 1..20: mol_type = other DNA
tttaaacccctgtttctact

SEQ ID NO: 38 - DNA, 25 bp, synthetic construct
  source 1..25: mol_type = other DNA
ctttcaatatctacctgctttcgct

SEQ ID NO: 39 - DNA, 25 bp, synthetic construct
  source 1..25: mol_type = other DNA
ctttcaatatctacctgtttccact

SEQ ID NO: 40 - DNA, 25 bp, synthetic construct
  source 1..25: mol_type = other DNA
ctttcaatatctacctgcttttgct

SEQ ID NO: 41 - DNA, 25 bp, synthetic construct
  source 1..25: mol_type = other DNA
ctttcaatatctacctgtttctact

SEQ ID NO: 42 - DNA, 26 bp, synthetic construct
  source 1..26: mol_type = other DNA
tatgtctttgtcaccctgcttttgct

SEQ ID NO: 43 - DNA, 26 bp, synthetic construct
  source 1..26: mol_type = other DNA
tatgtctttgtcaccctgtttctact

SEQ ID NO: 44 - DNA, 33 bp, synthetic construct
  source 1..33: mol_type = other DNA
tttgtggatattagaaaatgctctgcttctgct

SEQ ID NO: 45 - DNA, 33 bp, synthetic construct
  source 1..33: mol_type = other DNA
tttgtggatattagaaaatgctctgtttctact

SEQ ID NO: 46 - DNA, 21 bp, synthetic construct
  source 1..21: mol_type = other DNA
cttaaaggctccgcttctgct

SEQ ID NO: 47 - DNA, 21 bp, synthetic construct
  source 1..21: mol_type = other DNA
cttaaaggctccgtttctact

SEQ ID NO: 48 - DNA, 23 bp, synthetic construct
  source 1..23: mol_type = other DNA
cttgaaaacgctccgcttctgct

SEQ ID NO: 49 - DNA, 23 bp, synthetic construct
  source 1..23: mol_type = other DNA
cttgaaaacgctccgtttctact

SEQ ID NO: 50 - DNA, 29 bp, synthetic construct
  source 1..29: mol_type = other DNA
tatgacaaatcaaacgcaccgcttctgct

SEQ ID NO: 51 - DNA, 29 bp, synthetic construct
  source 1..29: mol_type = other DNA
tatgacaaatcaaacgcaccgtttctact

SEQ ID NO: 52 - DNA, 60 bp, synthetic construct
  source 1..60: mol_type = other DNA
tttgattttcagttcttttactggtgcttgaagttcacaagaaaatgctgtgcttctgct

SEQ ID NO: 53 - DNA, 60 bp, synthetic construct
  source 1..60: mol_type = other DNA
tttgattttcagttcttttactggtgcttgaagttcacaagaaaatgctgtgtttctact

SEQ ID NO: 54 - DNA, 46 bp, synthetic construct
  source 1..46: mol_type = other DNA
ttttggcctatttgcctcagttttgagaagatgctctgcttctgct

SEQ ID NO: 55 - DNA, 46 bp, synthetic construct
  source 1..46: mol_type = other DNA
ttttggcctatttgcctcagttttgagaagatgctctgtttctact

SEQ ID NO: 56 - DNA, 24 bp, synthetic construct
  source 1..24: mol_type = other DNA
tttaagaaagtgcgtgcttctgct

SEQ ID NO: 57 - DNA, 24 bp, synthetic construct
  source 1..24: mol_type = other DNA
tttaagaaagtgcgtgtttctact

SEQ ID NO: 58 - DNA, 44 bp, synthetic construct
  source 1..44: mol_type = other DNA
ttttccctgtttgccagtgactaaacaaatcctctgcttctgct

SEQ ID NO: 59 - DNA, 44 bp, synthetic construct
  source 1..44: mol_type = other DNA
ttttccctgtttgccagtgactaaacaaatcctctgcttctact

SEQ ID NO: 60 - DNA, 1493 bp, synthetic construct
  source 1..1493: mol_type = other DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaacgaacaaattaaa

SEQ ID NO: 61 - DNA, 1493 bp, synthetic construct
  source 1..1493: mol_type = other DNA
atattaggtttttacctacccaggaaaagccaaccaacctcgatctcttgtagatctgttctctaaccggattttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaccggataaattaaa

SEQ ID NO: 62 - DNA, 1493 bp, synthetic construct
  source 1..1493: mol_type = other DNA
atattaggtttttacctacccaggaaaagccaaccaacctcgatctcttgtagatctgttctctaaccggattttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttgctgccaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaacgaacaaattaaa

SEQ ID NO: 63 - DNA, 1493 bp, synthetic construct
  source 1..1493: mol_type = other DNA
atattaggtttttacctacccaggaaaagccaaccaacctcgatctcttgtagatctgttctctaaccggattttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttgctgccaacggtaataagggagccggtggtcatagctatggcatcgatctaaagtcttatgacttaggtgacgagcttggcactgatcccattgaagattatgaacaaaactggaacactgccgctggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaccggataaattaaa

SEQ ID NO: 64 - DNA, 1493 bp, synthetic construct
  source 1..1493: mol_type = other DNA
atattaggtttttacctacccaggaaaagccaaccaacctcgatctcttgtagatctgttctctaaccggattttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactgccgctggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaccggataaattaaa

SEQ ID NO: 65 - DNA, 1493 bp, synthetic construct
  source 1..1493: mol_type = other DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttgctgccaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaacgaacaaattaaa

SEQ ID NO: 66 - DNA, 1493 bp, synthetic construct
  source 1..1493: mol_type = other DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttgctgccaacggtaataagggagccggtggtcatagctatggcatcgatctaaagtcttatgacttaggtgacgagcttggcactgatcccattgaagattatgaacaaaactggaacactgccgctggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaacgaacaaattaaa

SEQ ID NO: 67 - DNA, 1493 bp, synthetic construct
  source 1..1493: mol_type = other DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactgccgctggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaacgaacaaattaaa

SEQ ID NO: 68 - DNA, 1501 bp, synthetic construct
  source 1..1501: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 69 - DNA, 1501 bp, synthetic construct
  source 1..1501: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 70 - DNA, 1513 bp, synthetic construct
  source 1..1513: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttctcccattttagtgacgttcgtgttgttttagatttcatctaaccggataaactaaa

SEQ ID NO: 71 - DNA, 1522 bp, synthetic construct
  source 1..1522: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttctttgactcccattttagttttgacgttcgtgttgttttagatttcatctaaccggataaactaaa

SEQ ID NO: 72 - DNA, 1529 bp, synthetic construct
  source 1..1529: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttgaactttgactcccattttagtttatcctgacgttcgtgttgttttagatttcatctaaccggataaactaaa

SEQ ID NO: 73 - DNA, 1536 bp, synthetic construct
  source 1..1536: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttcaaacagaactttgactcccattttagtttatccgtgacgttcgtgttgttttagatttcatctaaccggataaactaaa

SEQ ID NO: 74 - DNA, 1552 bp, synthetic construct
  source 1..1552: mol_type = other DNA
taaacctttaatctgatgagtccgtgaggacgaaacccggagtcccgggtcattaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 75 - DNA, 1753 bp, synthetic construct
  source 1..1753: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatccttaaaactgacgttcgtgttgttttagatttcatctaaccggataaactaaa

SEQ ID NO: 76 - DNA, 1771 bp, synthetic construct
  source 1..1771: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaacggccgatatcacggccgaagatcacattggcacccgcaatccttaaaactgacgttcgtgttgttttagatttcatctaaccggataaactaaa

SEQ ID NO: 77 - DNA, 1787 bp, synthetic construct
  source 1..1787: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaagtccaccacggccgatatcacggccgtggtggacaagatcacattggcacccgcaatccttaaaactgacgttcgtgttgttttagatttcatctaaccggataaactaaa

SEQ ID NO: 78 - DNA, 1801 bp, synthetic construct
  source 1..1801: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaccctgcggtccaccacggccgatatcacggccgtggtggaccgcagggaagatcacattggcacccgcaatccttaaaactgacgttcgtgttgttttagatttcatctaaccggataaactaaa

SEQ ID NO: 79 - DNA, 1754 bp, synthetic construct
  source 1..1754: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgtttaaacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatccttaaaactgacgttcgtgttgttttagatttcatctaaccggataaactaaa

SEQ ID NO: 80 - DNA, 1762 bp, synthetic construct
  source 1..1762: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatccttaaaaccaccatggtgacgttcgtgttgtttaagatttcatctaaccggataaactacaa

SEQ ID NO: 81 - DNA, 1501 bp, synthetic construct
  source 1..1501: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgtgcgaaagcaaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgtttttcatttcatctaaccggataaactaaa

SEQ ID NO: 82 - DNA, 1510 bp, synthetic construct
  source 1..1510: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacattttagtttatccggttagtttagatttcatctaaccggataaactaaa

SEQ ID NO: 83 - DNA, 1526 bp, synthetic construct
  source 1..1526: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacattttagtttatccggttagatgaaatccgtgttgttttagatttcatctaaccggataaactaaa

SEQ ID NO: 84 - DNA, 1495 bp, synthetic construct
  source 1..1495: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaaaactaaa

SEQ ID NO: 85 - DNA, 1501 bp, synthetic construct
  source 1..1501: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttgctgccaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 86 - DNA, 1501 bp, synthetic construct
  source 1..1501: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttgctgccaacggtaataaaggagctggtggccatagttacggcgccgatctaaagtcatttgacttaggcgacgagcttggcactgatccttatgaagattttcaagaaaactggaacactgccgctagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 87 - DNA, 1502 bp, synthetic construct
  source 1..1502: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgctagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaaccggataaactaaa

SEQ ID NO: 88 - DNA, 1530 bp, synthetic construct
  source 1..1530: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgaccaccatgcggggcgacctggccctcctgaagctgctcgacctgcggtttcatctaaccggataaactaaa

SEQ ID NO: 89 - DNA, 1533 bp, synthetic construct
  source 1..1533: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgaccaccatgaagatcggcctgggcaggcagaagccccctaaagccaccaagtttcatctaaccggataaactaaa

SEQ ID NO: 90 - DNA, 1492 bp, synthetic construct
  source 1..1492: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgaccaccatgtttcatctaaccggataaactaaaa

SEQ ID NO: 91 - DNA, 1210 bp, synthetic construct
  source 1..1210: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgaggtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 92 - DNA, 1201 bp, synthetic construct
  source 1..1201: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 93 - DNA, 1201 bp, synthetic construct
  source 1..1201: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 94 - DNA, 1231 bp, synthetic construct
  source 1..1231: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggttgacgttcgtgttgttttagatttcatctaaccggataaactaaa

SEQ ID NO: 95 - DNA, 1471 bp, synthetic construct
  source 1..1471: mol_type = other DNA
attaaaggtttatagatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 96 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 97 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtatacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 98 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 99 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 100 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatgacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 101 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 102 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgttcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 103 - DNA, 1471 bp, synthetic construct
  source 1..1471: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaccggataaactaaa

SEQ ID NO: 104 - DNA, 1513 bp, synthetic construct
  source 1..1513: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttctcccattttagtgacgttcgtgttgttttagatttcatctaaacgaacaaactaaa

SEQ ID NO: 105 - DNA, 1522 bp, synthetic construct
  source 1..1522: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttctttgactcccattttagttttgacgttcgtgttgttttagatttcatctaaacgaacaaactaaa

SEQ ID NO: 106 - DNA, 1529 bp, synthetic construct
  source 1..1529: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttgaactttgactcccattttagtttatcctgacgttcgtgttgttttagatttcatctaaacgaacaaactaaa

SEQ ID NO: 107 - DNA, 1536 bp, synthetic construct
  source 1..1536: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttcaaacagaactttgactcccattttagtttatccgtgacgttcgtgttgttttagatttcatctaaacgaacaaactaaa

SEQ ID NO: 108 - DNA, 1552 bp, synthetic construct
  source 1..1552: mol_type = other DNA
taaacctttaatctgatgagtccgtgaggacgaaacccggagtcccgggtcattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 109 - DNA, 1753 bp, synthetic construct
  source 1..1753: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatccttaaaactgacgttcgtgttgttttagatttcatctaaacgaacaaactaaa

SEQ ID NO: 110 - DNA, 1771 bp, synthetic construct
  source 1..1771: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaacggccgatatcacggccgaagatcacattggcacccgcaatccttaaaactgacgttcgtgttgttttagatttcatctaaacgaacaaactaaa

SEQ ID NO: 111 - DNA, 1787 bp, synthetic construct
  source 1..1787: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaagtccaccacggccgatatcacggccgtggtggacaagatcacattggcacccgcaatccttaaaactgacgttcgtgttgttttagatttcatctaaacgaacaaactaaa

SEQ ID NO: 112 - DNA, 1801 bp, synthetic construct
  source 1..1801: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaccctgcggtccaccacggccgatatcacggccgtggtggaccgcagggaagatcacattggcacccgcaatccttaaaactgacgttcgtgttgttttagatttcatctaaacgaacaaactaaa

SEQ ID NO: 113 - DNA, 1754 bp, synthetic construct
  source 1..1754: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgtttaaacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatccttaaaactgacgttcgtgttgttttagatttcatctaaacgaacaaactaaa

SEQ ID NO: 114 - DNA, 1762 bp, synthetic construct
  source 1..1762: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattgacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatccttaaaaccaccatggtgacgttcgtgttgtttaagatttcatctaaacgaacaaactacaa

SEQ ID NO: 115 - DNA, 1501 bp, synthetic construct
  source 1..1501: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgtgcgaaagcaaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgtttttcatttcatctaaacgaacaaactaaa

SEQ ID NO: 116 - DNA, 1501 bp, synthetic construct
  source 1..1501: mol_type = other DNA
attaaaggtttataccttcccaggtaacaaaccaaccaactttccatctcttgaagatgggttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 117 - DNA, 1501 bp, synthetic construct
  source 1..1501: mol_type = other DNA
attaaaggtttataccttcccaggtaacaaaccaaccaactatggatctcttgaagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 118 - DNA, 1501 bp, synthetic construct
  source 1..1501: mol_type = other DNA
attaaaggtttataccttcccaggtaacaaaccaaccaactttcgatctcttgtagatctgtaccatggacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 119 - DNA, 1510 bp, synthetic construct
  source 1..1510: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacattttagtttgttcgtttagtttagatttcatctaaacgaacaaactaaa

SEQ ID NO: 120 - DNA, 1526 bp, synthetic construct
  source 1..1526: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacattttagtttgttcgtttagatgaaatccgtgttgttttagatttcatctaaacgaacaaactaaa

SEQ ID NO: 121 - DNA, 1495 bp, synthetic construct
  source 1..1495: mol_type = other DNA
attaaaggtttataccttcccaggtaacaaaccaaccaactttcgatctcttgtagatctgttctctaaacggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaaaactaaa

SEQ ID NO: 122 - DNA, 1501 bp, synthetic construct
  source 1..1501: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttgctgccaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 123 - DNA, 1501 bp, synthetic construct
  source 1..1501: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttgctgccaacggtaataaaggagctggtggccatagttacggcgccgatctaaagtcatttgacttaggcgacgagcttggcactgatccttatgaagattttcaagaaaactggaacactgccgctagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 124 - DNA, 1501 bp, synthetic construct
  source 1..1501: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgctagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaacgaacaaactaaa

SEQ ID NO: 125 - DNA, 1530 bp, synthetic construct
  source 1..1530: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgaccaccatgcggggcgacctggccctcctgaagctgctcgacctgcggtttcatctaaacgaacaaactaaa

SEQ ID NO: 126 - DNA, 1533 bp, synthetic construct
  source 1..1533: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgaccaccatgaagatcggcctgggcaggcagaagccccctaaagccaccaagtttcatctaaacgaacaaactaaa

SEQ ID NO: 127 - DNA, 1492 bp, synthetic construct
  source 1..1492: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgaccaccatgtttcatctaaacgaacaaactaaaa

SEQ ID NO: 128 - DNA, 80 bp, synthetic construct
  source 1..80: mol_type = other DNA
tttaatttatccggttagagaacagatctacaagagatcgaggttggttggcttttcctgggtaggtaaaaacctaatat

SEQ ID NO: 129 - DNA, 370 bp, synthetic construct
  source 1..370: mol_type = other DNA
actcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 130 - DNA, 370 bp, synthetic construct
  source 1..370: mol_type = other DNA
actcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 131 - DNA, 340 bp, synthetic construct
  source 1..340: mol_type = other DNA
actcatgcagaccacacaaggcagatgggcatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 132 - DNA, 340 bp, synthetic construct
  source 1..340: mol_type = other DNA
actcatgcagaccacacaaggcagatgggctatataaacgttttcgcttttccgtttacgatatatagtctactcttgtgcagaatgaattctaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 133 - DNA, 340 bp, synthetic construct
  source 1..340: mol_type = other DNA
actcatgcagaccacacaaggcagatgggctatataaacgttttcgcttttccgtttacgatatatagtctactcttgtgcagaatgaattctcgtaactacatagcacaagtagatgtagttaactttaatctcacatagcaacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 134 - DNA, 340 bp, synthetic construct
  source 1..340: mol_type = other DNA
actcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 135 - DNA, 340 bp, synthetic construct
  source 1..340: mol_type = other DNA
actcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcggcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 136 - DNA, 83 bp, synthetic construct
  source 1..83: mol_type = other DNA
tttagtttatccggttagagaacagatctacaagagatcgaaagttggttggtttgttacctgggaaggtataaacctttaat

SEQ ID NO: 137 - DNA, 1607 bp, synthetic construct
  source 1..1607: mol_type = other DNA
ttttttttttgtcattctcctaagaagctattaaaatcacatggggatagcactactaaaattaattttacacattagggctcttccatataggcagctctccctagcattattcactgtaccctcgatcgtactccgcgtggcctcgatgaaaatgtggtggctctttcaagtcctccctaatgttacacattgattaaagattgctatgtgagattaaagttaactaaacctacttgtgctgtttagttacgagaattcattctgcacaagagtagactatgtatcgtaaacggaattgcgaaaacgtttacatagcccatctgccttgtgtggtcatcatgagtttaggcctgagttgagtcagcactgctcatggattgttgcaattgtttggagaaatcatccaaatctgcagcaggaagaagagtcacagtttgctgtttcttctgtctctgcggtaaggcttgagtttcatcagccttcttctttttgtcctttttaggctctgttggtgggaatgttttgtatgcgtcaatatgcttattcagcaaaatgacttgatctttgaaatttggatctttgtcatccaatttgatggcacctgtgtaggtcaaccacgttcccgaaggtgtgacttccatgccaatgcgcgacattccgaagaacgctgaagcgctgggggcaaattgtgcaatttgcggccaatgtttgtaatcagttccttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccacgaattcgtctggtagctcttcggtagtagccaatttggtcatctggactgctattggtgttaattggaacgccttgtcctcgagggaatttaaggtcttccttgccatgttgagtgagagcggtgaaccaagacgcagtattattgggtaaaccttggggccgacgttgttttgatcgcgccccactgcgttctccattctggttactgccagttgaatctgagggtccaccaaacgtaatgcggggtgcatttcgctgattttggggtccattatcagatta

SEQ ID NO: 138 - DNA, 352 bp, synthetic construct
  source 1..352: mol_type = other DNA
tttttttttttttttgtcattctcctaagaagctattaaaatcacatggggatagcactactaaaattaattttacacattagggctcttccatataggcagctctccctagcattgttcactgtacactcgatcgtactccgcgtggcctcggtgaaaatgtggtggctctttcaagtcctccctaatgttacacactgattaaagattgctatgtgagattaaagttaactacatctacttgtgctatgtagttacgagaattcattctgcacaagagtagactatatatcgtaaacggaaaagcgaaaacgtttatatagcccatctgccttgtgtggtctgcatgagt

SEQ ID NO: 139 - DNA, 1607 bp, synthetic construct
  source 1..1607: mol_type = other DNA
ttttttttttgtcattctcctaagaagctattaaaatcacatggggatagcactactaaaattaattttacacattagggctcttccatataggcagctctccctagcattgttcactgtacactcgatcgtactccgcgtggcctcggtgaaaatgtggtggctctttcaagtcctccctaatgttacacactgattaaagattgctatgtgagattaaagttaactacatctacttgtgctatgtagttacgagaattcattctgcacaagagtagactatatatcgtaaacggaaaagcgaaaacgtttatatagcccatctgccttgtgtggtctgcatgagtttaggcctgagttgagtcagcactgctcatggattgttgcaattgtttggagaaatcatccaaatctgcagcaggaagaagagtcacagtttgctgtttcttctgtctctgcggtaaggcttgagtttcatcagccttcttctttttgtcctttttaggctctgttggtgggaatgttttgtatgcgtcaatatgcttattcagcaaaatgacttgatctttgaaatttggatctttgtcatccaatttgatggcacctgtgtaggtcaaccacgttcccgaaggtgtgacttccatgccaatgcgcgacattccgaagaacgctgaagcgctgggggcaaattgtgcaatttgcggccaatgtttgtaatcagttccttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccacgaattcgtctggtagctcttcggtagtagccaatttggtcatctggactgctattggtgttaattggaacgccttgtcctcgagggaatttaaggtcttccttgccatgttgagtgagagcggtgaaccaagacgcagtattattgggtaaaccttggggccgacgttgttttgatcgcgccccactgcgttctccattctggttactgccagttgaatctgagggtccaccaaacgtaatgcggggtgcatttcgctgattttggggtccattatcagatta

SEQ ID NO: 140 - DNA, 1528 bp, synthetic construct
  source 1..1528: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaaaactaaattttttttttttttttttttttttttttttttt

SEQ ID NO: 141 - DNA, 1633 bp, synthetic construct
  source 1..1633: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaaaactaaatcgaggcggatcgatccgagctctcccttagccatccgagtggacgacgtcctccttcggatgcccaggtcggaccgcgaggaggtggagatgccatgccgacccttttttttttttttttttttttttttttttttt

SEQ ID NO: 142 - DNA, 1610 bp, synthetic construct
  source 1..1610: mol_type = other DNA
nnnttttttttttgtcattctcctaagaagctattaaaatcacatggggatagcactactaaaattaattttacacattagggctcttccatataggcagctctccctagcattgttcactgtacactcgatcgtactccgcgtggcctcggtgaaaatgtggtggctctttcaagtcctccctaatgttacacactgattaaagattgctatgtgagattaaagttaactacatctacttgtgctatgtagttacgagaattcattctgcacaagagtagactatatatcgtaaacggaaaagcgaaaacgtttatatagcccatctgccttgtgtggtctgcatgagtttaggcctgagttgagtcagcactgctcatggattgttgcaattgtttggagaaatcatccaaatctgcagcaggaagaagagtcacagtttgctgtttcttctgtctctgcggtaaggcttgagtttcatcagccttcttctttttgtcctttttaggctctgttggtgggaatgttttgtatgcgtcaatatgcttattcagcaaaatgacttgatctttgaaatttggatctttgtcatccaatttgatggcacctgtgtaggtcaaccacgttcccgaaggtgtgacttccatgccaatgcgcgacattccgaagaacgctgaagcgctgggggcaaattgtgcaatttgcggccaatgtttgtaatcagttccttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccacgaattcgtctggtagctcttcggtagtagccaatttggtcatctggactgctattggtgttaattggaacgccttgtcctcgagggaatttaaggtcttccttgccatgttgagtgagagcggtgaaccaagacgcagtattattgggtaaaccttggggccgacgttgttttgatcgcgccccactgcgttctccattctggttactgccagttgaatctgagggtccaccaaacgtaatgcggggtgcatttcgctgattttggggtccattatcagatta

SEQ ID NO: 143 - DNA, 1528 bp, synthetic construct
  source 1..1528: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaaaactaaattttttttttttttttttttttttttttttttt

SEQ ID NO: 144 - DNA, 1633 bp, synthetic construct
  source 1..1633: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagatttttgacgttcgtgttgttttagatttcatctaaaaactaaatcgaggcggatcgatccgagctctcccttagccatccgagtggacgacgtcctccttcggatgcccaggtcggaccgcgaggaggtggagatgccatgccgacccttttttttttttttttttttttttttttttttt

SEQ ID NO: 145 - DNA, 1501 bp, synthetic construct
  source 1..1501: mol_type = other DNA
tttagtttgttcgtttagatgaaatctaaaacaacacgaacgtcaaaaatctccataaacgatatgttcgaaggcatagccttctaatttataccgttcaatgaattcatccatagctaattctaagaaatcaatttccatttgactcctgggtttaaattcttgtaaatttctactctgagtaaagtaagtttcaggtaattgttggacaacaccatcaactttcttataataattgaactgtgtttttacggcttctccaattaatgtgactccattaagactagcttgtttgggacctacagatggttgtaaacctttaacactaccttctgtaataagaacaccattacgggcatttctaaataagtctacttgaccatcaactctaccatcaaaaaagacagtgagtggtgcacaaatcgtttcagttggtttcttggctatgtcagtcatagaacaaacaccaatagtagatatatgtgctggagcatctcttttgtagtcccagatcacagtattagcagcaatgtccacacccaaattattgagtattttcacctctggtactggtttaatgttgcgcttagcccaaagctcaaatgctacattaacaggtaatgttgttttattttcaaacaattctacatcaacaccatcaacttttgtgtaaacagtgttattaatgatagaaactggtacttcaccctgtcgcatgagttcacgggtaacaccactgctatgtttagtgttccagttttcttgaaaatcttcataaggatcagtgccaagctcgtcgcctaagtcaaatgactttagatcggcgccgtaactatggccaccagctcctttattaccgttcttacgaagaagaaccttgcggtaagccactggtatttcgcccacatgagggacaaggacaccaagtgtctcaccactacgaccgtactgaatgccttcgagttctgctaccagctcaaccataacatgaccatgaggtgcagttcgagcatccgaacgtttgatgaacacatagggctgttcaagttgaggcaaaacgcctttttcaacttctactaagccacaagtgccatctttaagatgttgacgtgcctctgataagacctcctccacggagtctccaaagccacgtacgagcacgtcgcgaacctgtaaaacaggcaaactgagttggacgtgtgttttctcgttgaaaccagggacaaggctctccatcttacctttcggtcacacccggacgaaacctagatgtgctgatgatcggctgcaacacggacgaaaccgtaagcagcctgcagaagatagacgagttactcgtgtcctgtcaacgacagtaattagttattaattatactgcgtgagtgcactaagcatgcagccgagtgacagccacacagattttaaaatccggttagagaacagatctacaagagatcgaaagttggttggtttgttacctgggaaggtataaacctttaat

SEQ ID NO: 146 - DNA, 1501 bp, synthetic construct
  source 1..1501: mol_type = other DNA
tttagtttatccggttagatgaaatctaaaacaacacgaacgtcaaaaatctccataaacgatatgttcgaaggcatagccttctaatttataccgttcaatgaattcatccatagctaattctaagaaatcaatttccatttgactcctgggtttaaattcttgtaaatttctactctgagtaaagtaagtttcaggtaattgttggacaacaccatcaactttcttataataattgaactgtgtttttacggcttctccaattaatgtgactccattaagactagcttgtttgggacctacagatggttgtaaacctttaacactaccttctgtaataagaacaccattacgggcatttctaaataagtctacttgaccatcaactctaccatcaaaaaagacagtgagtggtgcacaaatcgtttcagttggtttcttggctatgtcagtcatagaacaaacaccaatagtagatatatgtgctggagcatctcttttgtagtcccagatcacagtattagcagcaatgtccacacccaaattattgagtattttcacctctggtactggtttaatgttgcgcttagcccaaagctcaaatgctacattaacaggtaatgttgttttattttcaaacaattctacatcaacaccatcaacttttgtgtaaacagtgttattaatgatagaaactggtacttcaccctgtcgcatgagttcacgggtaacaccactgctatgtttagtgttccagttttcttgaaaatcttcataaggatcagtgccaagctcgtcgcctaagtcaaatgactttagatcggcgccgtaactatggccaccagctcctttattaccgttcttacgaagaagaaccttgcggtaagccactggtatttcgcccacatgagggacaaggacaccaagtgtctcaccactacgaccgtactgaatgccttcgagttctgctaccagctcaaccataacatgaccatgaggtgcagttcgagcatccgaacgtttgatgaacacatagggctgttcaagttgaggcaaaacgcctttttcaacttctactaagccacaagtgccatctttaagatgttgacgtgcctctgataagacctcctccacggagtctccaaagccacgtacgagcacgtcgcgaacctgtaaaacaggcaaactgagttggacgtgtgttttctcgttgaaaccagggacaaggctctccatcttacctttcggtcacacccggacgaaacctagatgtgctgatgatcggctgcaacacggacgaaaccgtaagcagcctgcagaagatagacgagttactcgtgtcctgtcaacgacagtaattagttattaattatactgcgtgagtgcactaagcatgcagccgagtgacagccacacagattttaaaatccggttagagaacagatctacaagagatcgaaagttggttggtttgttacctgggaaggtataaacctttaat

SEQ ID NO: 147 - DNA, 957 bp, synthetic construct
  source 1..957: mol_type = other DNA
ggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 148 - DNA, 98 bp, synthetic construct
  source 1..98: mol_type = other DNA
acgcgaaaaaatgcgtacaacaaacttgcataaaccaaaaaaatggggcaaataagaatttgataagtaccacttaaatttaactcccttgcttagcg

SEQ ID NO: 149 - DNA, 53 bp, synthetic construct
  source 1..53: mol_type = other DNA
acgcgaaaaaatgcgtacaacaaacttgcataaaccaaaaaaatggggcaaat

SEQ ID NO: 150 - DNA, 55 bp, synthetic construct
  source 1..55: mol_type = other DNA
acgagaaaaaaagtgtcaaaaactaatatctcgtaaccaaaaaaatggggcaaat

SEQ ID NO: 151 - DNA, 56 bp, synthetic construct
  source 1..56: mol_type = other DNA
acgcgaaaaaatgcgtacaacaaacttgcataaaccaaaaaaatggggcaaatctt

SEQ ID NO: 152 - DNA, 58 bp, synthetic construct
  source 1..58: mol_type = other DNA
acgagaaaaaaagtgtcaaaaactaatatctcgtaaccaaaaaaatggggcaaatctt

SEQ ID NO: 153 - DNA, 54 bp, synthetic construct
  source 1..54: mol_type = other DNA
ggggcaaataagaatttgataagtaccacttaaatttaactcccttgcttagcg

SEQ ID NO: 154 - DNA, 227 bp, synthetic construct
  source 1..227: mol_type = other DNA
tgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 155 - DNA, 52 bp, synthetic construct
  source 1..52: mol_type = other DNA
agttattaaaaataaattacgagatattagtttttgacactttttttctcgt

SEQ ID NO: 156 - DNA, 112 bp, synthetic construct
  source 1..112: mol_type = other DNA
tgaataaagatcttataataaaaattcccatagctatacactaacactgtattcaattatagttattaaaaattaaaaatcgtacgattttttaaataacttttagtgaact

SEQ ID NO: 157 - DNA, 171 bp, synthetic construct
  source 1..171: mol_type = other DNA
tgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggt

SEQ ID NO: 158 - DNA, 227 bp, synthetic construct
  source 1..227: mol_type = other DNA
acgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 159 - DNA, 248 bp, synthetic construct
  source 1..248: mol_type = other DNA
agctgacacgctgtcctctggacgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 160 - DNA, 278 bp, synthetic construct
  source 1..278: mol_type = other DNA
agctgacacgctgtcctctggcgacctgtcgtcggagaggttgggcctccgacgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 161 - DNA, 328 bp, synthetic construct
  source 1..328: mol_type = other DNA
agctgacacgctgtcctctggcgacctgtcgtcggagaggttgggcctccggatgcgcgcggggctctggcctcacggtgaccggctagccggccgcgctcacgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 162 - DNA, 461 bp, synthetic construct
  source 1..461: mol_type = other DNA
gtgtcctctggcgacctgtcggctgtcctctggcgacctgtcgtcggagaggttgggccgtcgttcggagaggttgggcctccggatgcgcgcagctgacacgctgtcctctggcgacctgtgggctctggcctcacggtgaccggctagccggccgcgctccgtcggagaggttgggcctccggatgcgcgcggggctctggcctcacggtgaccggctagccggccgcgctcacgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 163 - DNA, 230 bp, synthetic construct
  source 1..230: mol_type = other DNA
aggacgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 164 - DNA, 227 bp, synthetic construct
  source 1..227: mol_type = other DNA
aggagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 165 - DNA, 167 bp, synthetic construct
  source 1..167: mol_type = other DNA
acgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataact

SEQ ID NO: 166 - DNA, 52 bp, synthetic construct
  source 1..52: mol_type = other DNA
acgagaaaaaaagtgtcaaaaactaatatctcgtaatttatttttaataact

SEQ ID NO: 167 - DNA, 112 bp, synthetic construct
  source 1..112: mol_type = other DNA
agttcactaaaagttatttaaaaaatcgtacgatttttaatttttaataactataattgaatacagtgttagtgtatagctatgggaatttttattataagatctttattca

SEQ ID NO: 168 - DNA, 171 bp, synthetic construct
  source 1..171: mol_type = other DNA
accaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattca

SEQ ID NO: 169 - DNA, 98 bp, synthetic construct
  source 1..98: mol_type = other DNA
cgctaagcaagggagttaaatttaagtggtacttatcaaattcttatttgccccatttttttggtttatgcaagtttgttgtacgcattttttcgcgt

SEQ ID NO: 170 - DNA, 100 bp, synthetic construct
  source 1..100: mol_type = other DNA
cgctaagcaagggagttaaatttaagtggtacttatcaaattcttatttgccccatttttttggttacgagatattagtttttgacactttttttctcgt

SEQ ID NO: 171 - DNA, 121 bp, synthetic construct
  source 1..121: mol_type = other DNA
cgctaagcaagggagttaaatttaagtggtacttatcaaattcttatttgccccatttttttggttacgagatattagtttttgacactttttttctcgtagctgacacgctgtcctctgg

SEQ ID NO: 172 - DNA, 151 bp, synthetic construct
  source 1..151: mol_type = other DNA
cgctaagcaagggagttaaatttaagtggtacttatcaaattcttatttgccccatttttttggttacgagatattagtttttgacactttttttctcgtagctgacacgctgtcctctggcgacctgtcgtcggagaggttgggcctccg

SEQ ID NO: 173 - DNA, 201 bp, synthetic construct
  source 1..201: mol_type = other DNA
cgctaagcaagggagttaaatttaagtggtacttatcaaattcttatttgccccatttttttggttacgagatattagtttttgacactttttttctcgtagctgacacgctgtcctctggcgacctgtcgtcggagaggttgggcctccggatgcgcgcggggctctggcctcacggtgaccggctagccggccgcgctc

SEQ ID NO: 174 - DNA, 374 bp, synthetic construct
  source 1..374: mol_type = other DNA
cgctaagcaagggagttaaatttaagtggtacttatcaaattcttatttgccccatttttttggttacgagatattagtttttgacactttttttctcgtctccggatgcgcgcagctgacacgctgtcctctggcggagacgacctgtcgttcggagaggttgggcctccggatgcgcgcagctgacacgctgtcctctggcgacctgtgggctctggcctcacggtgaccggctagccggccgcgctccgtcggagaggttgggcctccggatgcgcgcggggctctggcctcacggtgaccggctagccggccgcgctccggatgcgcgcggggctctggcctcacggtgaccggctagccggccgcgctc

SEQ ID NO: 175 - DNA, 100 bp, synthetic construct
  source 1..100: mol_type = other DNA
cgctaagcaagggagttaaatttaagtggtacttatcaaattcttatttgccccatttttttggttacgagatattagtttttgacactttttttctcct

SEQ ID NO: 176 - DNA, 56 bp, synthetic construct
  source 1..56: mol_type = other DNA
tatttgccccatttttttggttacgagatattagtttttgacactttttttctcgt

SEQ ID NO: 177 - DNA, 53 bp, synthetic construct
  source 1..53: mol_type = other DNA
atttgccccatttttttggtttatgcaagtttgttgtacgcattttttcgcgt

SEQ ID NO: 178 - DNA, 54 bp, synthetic construct
  source 1..54: mol_type = other DNA
cgctaagcaagggagttaaatttaagtggtacttatcaaattcttatttgcccc

SEQ ID NO: 179 - DNA, 119 bp, synthetic construct
  source 1..119: mol_type = other DNA
accaaacaagaggaaaaacttgtttggaatatataataatattaaatagtattttagggttaaagtaatactttaaagggacaagtcacagacatttgatcttagtataaatttttata

SEQ ID NO: 180 - DNA, 157 bp, synthetic construct
  source 1..157: mol_type = other DNA
tcttatatcttctcatctttattatctaatttgtttaaagagatgagttaacaagataagaaatccctttaactgactcataaaaacatagtaagaaaaacttacaacagacaagagtattaataatatatcgatatttcttaaactcttgtctggt

SEQ ID NO: 181 - DNA, 157 bp, synthetic construct
  source 1..157: mol_type = other DNA
accagacaagagtttaagaaatatcgatatattattaatactcttgtctgttgtaagtttttcttactatgtttttatgagtcagttaaagggatttcttatcttgttaactcatctctttaaacaaattagataataaagatgagaagatataaga

SEQ ID NO: 182 - DNA, 163 bp, synthetic construct
  source 1..163: mol_type = other DNA
aggaccagacaagagtttaagaaatatcgatatattattaatactcttgtctgttgtaagtttttcttactatgtttttatgagtcagttaaagggatttcttatcttgttaactcatctctttaaacaaattagataataaagatgagaagatataagatcc

SEQ ID NO: 183 - DNA, 157 bp, synthetic construct
  source 1..157: mol_type = other DNA
accagacaagagtttaagaaatatcgagatattattaatactcttgtctgttgtaagtttttcttactatgtttttatgagtcagttaaagggatttcttatcttgttaactcatctctttaaacaaattagataataaagatgagaagatataaga

SEQ ID NO: 184 - DNA, 119 bp, synthetic construct
  source 1..119: mol_type = other DNA
tataaaaatttatactaagatcaaatgtctgtgacttgtccctttaaagtattactttaaccctaaaatactatttaatattattatatattccaaacaagtttttcctcttgtttggt

SEQ ID NO: 185 - DNA, 110 bp, synthetic construct
  source 1..110: mol_type = other DNA
accaaacaagagaagagacttgtttggaaatataaatttaaattaaaattaacttaggattaaagacattgactagaaggtcaagaaaagggaactctataatttcaaaa

SEQ ID NO: 186 - DNA, 112 bp, synthetic construct
  source 1..112: mol_type = other DNA
ataaatacaataaagatatatcttaacctttatctttgagcccaaggatagacaaaaagtaagaaaaacatgtaatatatatataccaagcagagttcttctcttgtttggt

SEQ ID NO: 187 - DNA, 112 bp, synthetic construct
  source 1..112: mol_type = other DNA
accaaacaagagaagaactctgcttggtatatatatattacatgtttttcttactttttgtctatccttgggctcaaagataaaggttaagatatatctttattgtatttat

SEQ ID NO: 188 - DNA, 118 bp, synthetic construct
  source 1..118: mol_type = other DNA
aggaccaaacaagagaagaactctgcttggtatatatatattacatgtttttcttactttttgtctatccttgggctcaaagataaaggttaagatatatctttattgtatttatgtc

SEQ ID NO: 189 - DNA, 112 bp, synthetic construct
  source 1..112: mol_type = other DNA
accaaacaagagaagaactctgcttgggatatatatattacatgtttttcttactttttgtctatccttgggctcaaagataaaggttaagatatatctttattgtatttat

SEQ ID NO: 190 - DNA, 110 bp, synthetic construct
  source 1..110: mol_type = other DNA
ttttgaaattatagagttcccttttcttgaccttctagtcaatgtctttaatcctaagttaattttaatttaaatttatatttccaaacaagtctcttctcttgtttggt

SEQ ID NO: 191 - DNA, 53 bp, synthetic construct
  source 1..53: mol_type = other DNA
acgagaaaaaaacgcatataagacaacttccaaacaaaacgggacaagtgaaa

SEQ ID NO: 192 - DNA, 53 bp, synthetic construct
  source 1..53: mol_type = other DNA
acgcgaaaaaaacgcgtataaattaaattccaaacaaaacgggacaagtgaaa

SEQ ID NO: 193 - DNA, 241 bp, synthetic construct
  source 1..241: mol_type = other DNA
cagtgctgaacttaaaaagctaatcaaagtgacaggttacatgctgatgagtaagaaatgatggaaggaaattaccttaagaaattacttatagtgaaggcttaaaacttacatatccatgctgaagaatactaaagtgatagttacttaaacttaataatctgaatatgaagcacacaactagcaattagttatttaaaaatgaggaactaaaattggatgaatacggtttttttgccgt

SEQ ID NO: 194 - DNA, 241 bp, synthetic construct
  source 1..241: mol_type = other DNA
aaaatgatgaagatgacaaaatagatgacaacttcatactattctaaattaattatttgattatgcaattatatgatagttaattaaaataataatgataatgattaaccataatctcacacaactgagaaaataatcgtctaacagtttagttgatcattagttatttaaaattataaaatagtaactaactgataaaaaatcagaaattgaaattgaatgtatacggtttttttgccgt

SEQ ID NO: 195 - DNA, 241 bp, synthetic construct
  source 1..241: mol_type = other DNA
acggcaaaaaaaccgtattcatccaattttagttcctcatttttaaataactaattgctagttgtgtgcttcatattcagattattaagtttaagtaactatcactttagtattcttcagcatggatatgtaagttttaagccttcactataagtaatttcttaaggtaatttccttccatcatttcttactcatcagcatgtaacctgtcactttgattagctttttaagttcagcactg

SEQ ID NO: 196 - DNA, 241 bp, synthetic construct
  source 1..241: mol_type = other DNA
acggcaaaaaaaccgtatacattcaatttcaatttctgattttttatcagttagttactattttataattttaaataactaatgatcaactaaactgttagacgattattttctcagttgtgtgagattatggttaatcattatcattattattttaattaactatcatataattgcataatcaaataattaatttagaatagtatgaagttgtcatctattttgtcatcttcatcatttt

SEQ ID NO: 197 - DNA, 247 bp, synthetic construct
  source 1..247: mol_type = other DNA
aggacggcaaaaaaaccgtatacattcaatttcaatttctgattttttatcagttagttactattttataattttaaataactaatgatcaactaaactgttagacgattattttctcagttgtgtgagattatggttaatcattatcattattattttaattaactatcatataattgcataatcaaataattaatttagaatagtatgaagttgtcatctattttgtcatcttcatcatttttcc

SEQ ID NO: 198 - DNA, 151 bp, synthetic construct
  source 1..151: mol_type = other DNA
acggcaaaaaaaccgtatacattcaatttcaatttctgattttttatcagttagttactattttataattttaaataactaatgatcaactaaactgttagacgattattttctcagttgtgtgagattatggttaatcattatcattatt

SEQ ID NO: 199 - DNA, 157 bp, synthetic construct
  source 1..157: mol_type = other DNA
aggacggcaaaaaaaccgtatacattcaatttcaatttctgattttttatcagttagttactattttataattttaaataactaatgatcaactaaactgttagacgattattttctcagttgtgtgagattatggttaatcattatcattattacc

SEQ ID NO: 200 - DNA, 53 bp, synthetic construct
  source 1..53: mol_type = other DNA
tttcacttgtcccgttttgtttggaagttgtcttatatgcgtttttttctcgt

SEQ ID NO: 201 - DNA, 53 bp, synthetic construct
  source 1..53: mol_type = other DNA
tttcacttgtcccgttttgtttggaatttaatttatacgcgtttttttcgcgt

SEQ ID NO: 202 - DNA, 112 bp, synthetic construct
  source 1..112: mol_type = other DNA
accaaacaagggagaatatggatacgttaaaatatataacgtatttttaaaacttaggaaccaagacaaacacttttggtcttggtattggatcctcaagaaatatatcatc

SEQ ID NO: 203 - DNA, 98 bp, synthetic construct
  source 1..98: mol_type = other DNA
taaccttccaaatccaagaccaactgataacttatgttgatctaaggttcagttattaagaaaaacttaataacgattcttctttacccttgttcggt

SEQ ID NO: 204 - DNA, 98 bp, synthetic construct
  source 1..98: mol_type = other DNA
accgaacaagggtaaagaagaatcgttattaagtttttcttaataactgaaccttagatcaacataagttatcagttggtcttggatttggaaggtta

SEQ ID NO: 205 - DNA, 104 bp, synthetic construct
  source 1..104: mol_type = other DNA
aggaccgaacaagggtaaagaagaatcgttattaagtttttcttaataactgaaccttagatcaacataagttatcagttggtcttggatttggaaggttaaac

SEQ ID NO: 206 - DNA, 112 bp, synthetic construct
  source 1..112: mol_type = other DNA
gatgatatatttcttgaggatccaataccaagaccaaaagtgtttgtcttggttcctaagttttaaaaatacgttatatattttaacgtatccatattctcccttgtttggt

SEQ ID NO: 207 - DNA, 98 bp, synthetic construct
  source 1..98: mol_type = other DNA
cgaactaccacacccatcaccaatgaagcccgaacaagtctacatagatcagttattaagaaaaacttaataacgatctctctttacccttgttcggt

SEQ ID NO: 208 - DNA, 112 bp, synthetic construct
  source 1..112: mol_type = other DNA
accgaacaaggggaaatatggatacgtgttaaaaaactgcgtatgtttaaaacttaggaaccaagacagtgacaattggtcttggtattggacaattgttcaaggttccaaa

SEQ ID NO: 209 - DNA, 98 bp, synthetic construct
  source 1..98: mol_type = other DNA
accgaacaagggtaaagagagatcgttattaagtttttcttaataactgatctatgtagacttgttcgggcttcattggtgatgggtgtggtagttcg

SEQ ID NO: 210 - DNA, 104 bp, synthetic construct
  source 1..104: mol_type = other DNA
aggaccgaacaagggtaaagagagatcgttattaagtttttcttaataactgatctatgtagacttgttcgggcttcattggtgatgggtgtggtagttcgatt

SEQ ID NO: 211 - DNA, 112 bp, synthetic construct
  source 1..112: mol_type = other DNA
tttggaaccttgaacaattgtccaataccaagaccaattgtcactgtcttggttcctaagttttaaacatacgcagttttttaacacgtatccatatttccccttgttcggt

SEQ ID NO: 212 - DNA, 1626 bp, synthetic construct
  source 1..1626: mol_type = other DNA
agcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacactggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtggaattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacattcctaacaacttatgagttcactttttttgcaggttgagggcagaggaagtctgctaacatgcggtgacgtcgaggagaatcctggcccaactagt

SEQ ID NO: 213 - DNA, 1591 bp, synthetic construct
  source 1..1591: mol_type = other DNA
agcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtggaattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatgagggcagaggaagtctgctaacatgcggtgacgtcgaggagaatcctggcccaactagt

SEQ ID NO: 214 - DNA, 1625 bp, synthetic construct
  source 1..1625: mol_type = other DNA
agcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacactggccagacgccaacaaagtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtggaattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacattcctaacaacttatgagttcactttttttgcaggggagggcagaggaagtctgctaacatgcggtgacgtcgaggagaatcctggcccaactagt

SEQ ID NO: 215 - DNA, 315 bp, synthetic construct
  source 1..315: mol_type = other DNA
aggtttttcacctctgcctaatcatctcttgttcatgtcctactgttcaagcctccaagctgtgccttgggtggctttggggcatggacattgacacgtataaagaatttggagcttctgtggagttactctcttttttgccttctgacttctttccttctattcaagatctcatcgacaccgcctctgcttaaccaaaatcaacgggactttccaaaatgtcgtaacaactccgccccattgacgcaaatgggcggtaggcgtgtacggtgggaggtctatataagcagagctcgtttagtgaaccgtgccacc

SEQ ID NO: 216 - DNA, 309 bp, synthetic construct
  source 1..309: mol_type = other DNA
aggtttttcacctctgcctaatcatctcttgttcatgtcctactgttcaagcctccaagctgtgccttgggtggctttggggcatggacattgacacgtataaagaatttggagcttctgtggagttactctcttttttgccttctgacttctttccttctattcaagatctcatcgacaccgcctctgcttaaccaaaatcaacgggactttccaaaatgtcgtaacaactccgccccattgacgcaaatgggcggtaggcgtgtacggtgggaggtctatataagcagagctcgtttagtgaaccgt

SEQ ID NO: 217 - DNA, 1054 bp, synthetic construct
  source 1..1054: mol_type = other DNA
agaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctacaaaaaaaaaaaaaaaaaaaaaaaaaaaaaaaaaaaaaaaaaaaaaaaaaaaaaaaaaaaaaaaaaaa

SEQ ID NO: 218 - DNA, 1090 bp, synthetic construct
  source 1..1090: mol_type = other DNA
caagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctacaaaaaaaaaaaaaaaaaaaaaaaaaaaaaaaaaaaaaaaaaaaaaaaaaaaaaaaaaaaaaaaaaaa

SEQ ID NO: 219 - DNA, 1192 bp, synthetic construct
  source 1..1192: mol_type = other DNA
cttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctacaaaaaaaaaaaaaaaaaaaaaaaaaaaaaaaaaaaaaaaaaaaaaaaaaaaaaaaaaaaaaaaaaaa

SEQ ID NO: 220 - DNA, 2609 bp, synthetic construct
  source 1..2609: mol_type = other DNA
ctgttgccctctggtttctccccaggttatccggcgggtttctgacatccggcgggtgactcacaaccccagaaacagacatatgcccctatcttatcaacacttccggaaactactgttgttagacgacgaggcaggccccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcctttcctcacattcatttacaggaggacattattaatagatgtcaacaatatgtgggccctcttacagttaatgaaaaaaggagattaaaattaattatgcctgctaggttctatcctaaccttaccaaatatttgcccttggacaaaggcattaaaccctattatcctgaacatgctgttaatcattacttcaaaactaggcattatttacatactctgtggaaggctggcattctatataagagagaaactacacgcagtgcctcattttgtgggtcaccatattcttgggaacaagagctacagcatgggaggttggtcttccaaacctcgacaaggcatttttcctgctggtggctccagttccggaacagtaaaccctgttccgactactgcctcacccatatcgtcaatcttctcgaggactggggaccctgcaccgaacatggagaacacaacatcagaattcctaggacccctgctcgtgttacaggcggggtttttcttgttgacaagaatcctcacaataccacagagtctagactcgtggtggacttctctcaattttctagggggagcacccgcgtgtcctggccaaaatttgcagtccccaacctccaatcactcaccaacctcttgtcctccgatttgacctggctatcgctggatgtgtctgcggcgttttatcatattcctcttcatcctgctgctatgcctcatcttcttgttggttcttctggactaccagggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcttgggctttcgcaagattcctatgggagtgggcctcagtccgtttctcctgactcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttaacccctaataaaaccaaacgttggggctactcccttaacttcatgggatatgtaattggaagttggggtactttaccgcaagaacatattgtacttaaaatcaaacaatgttttcgaaaactgcctgtaaatagacctattgattggaaagtatgtcagagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgccaggcaacggtcaggtctctgccaagtgtttgctgacgcaacccccactggatggggcttggctattggccatcgccgcatgcgtggaacctttgtggctcctctgccgatccatactgcggaactcctagcagcttgttttgctcgcagccggtctggagcaaaacttatcggaaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctatcgtccccttctacatctgccgttccggccgaccacggggcgcacctctctttacgcggtctccccgtctgtgccttctcatctgccggaccgtgtgcacttcgcttcacctctgcacgtcgcatggagaccaccgtgaacgccctccaggtcttgcccaaggtcttacataagaggactctcggactctcagcaatgtcaacgaccgaccttgaggcgtacttcaaagactgtttgtttaaggactgggaggagttgggggaggagattaggttaatgatctttgtactaggaggctgtaggcataaattggtctgttcaccagcaccatgcaactttttcacctctgcctaatcatctcttgttcatgtcctactgttcaagcctccaagctgtgccttgggtggctttggggcatggacatcgacccttataaagaatttggagcttcaaaaaaaaaaaaaaaaaaaaaaaaaaaaaaaaaaaaaaaaaaaaaaaaaaaaaaaaaaaaaaaaaaa

SEQ ID NO: 221 - DNA, 2724 bp, synthetic construct
  source 1..2724: mol_type = other DNA
acggttcactaaacgagctctgcttatatagacctcccaccgtacacgcctaccgcccatttgcgtcaatggggcggagttgttacgacattttggaaagtcccgttgattttggctgttgccctctggtttctccccaggttatccggcgggtttctgacatccggcgggtgactcacaaccccagaaacagacatatgcccctatcttatcaacacttccggaaactactgttgttagacgacgaggcaggccccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcctttcctcacattcatttacaggaggacattattaatagatgtcaacaatatgtgggccctcttacagttaatgaaaaaaggagattaaaattaattatgcctgctaggttctatcctaaccttaccaaatatttgcccttggacaaaggcattaaaccctattatcctgaacatgctgttaatcattacttcaaaactaggcattatttacatactctgtggaaggctggcattctatataagagagaaactacacgcagtgcctcattttgtgggtcaccatattcttgggaacaagagctacagcatgggaggttggtcttccaaacctcgacaaggcatttttcctgctggtggctccagttccggaacagtaaaccctgttccgactactgcctcacccatatcgtcaatcttctcgaggactggggaccctgcaccgaacatggagaacacaacatcagaattcctaggacccctgctcgtgttacaggcggggtttttcttgttgacaagaatcctcacaataccacagagtctagactcgtggtggacttctctcaattttctagggggagcacccgcgtgtcctggccaaaatttgcagtccccaacctccaatcactcaccaacctcttgtcctccgatttgacctggctatcgctggatgtgtctgcggcgttttatcatattcctcttcatcctgctgctatgcctcatcttcttgttggttcttctggactaccagggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcttgggctttcgcaagattcctatgggagtgggcctcagtccgtttctcctgactcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttaacccctaataaaaccaaacgttggggctactcccttaacttcatgggatatgtaattggaagttggggtactttaccgcaagaacatattgtacttaaaatcaaacaatgttttcgaaaactgcctgtaaatagacctattgattggaaagtatgtcagagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgccaggcaacggtcaggtctctgccaagtgtttgctgacgcaacccccactggatggggcttggctattggccatcgccgcatgcgtggaacctttgtggctcctctgccgatccatactgcggaactcctagcagcttgttttgctcgcagccggtctggagcaaaacttatcggaaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctatcgtccccttctacatctgccgttccggccgaccacggggcgcacctctctttacgcggtctccccgtctgtgccttctcatctgccggaccgtgtgcacttcgcttcacctctgcacgtcgcatggagaccaccgtgaacgccctccaggtcttgcccaaggtcttacataagaggactctcggactctcagcaatgtcaacgaccgaccttgaggcgtacttcaaagactgtttgtttaaggactgggaggagttgggggaggagattaggttaatgatctttgtactaggaggctgtaggcataaattggtctgttcaccagcaccatgcaactttttcacctctgcctaatcatctcttgttcatgtcctactgttcaagcctccaagctgtgccttgggtggctttggggcatggacatcgacccttataaagaatttggagcttcaaaaaaaaaaaaaaaaaaaaaaaaaaaaaaaaaaaaaaaaaaaaaaaaaaaaaaaaaaaaaaaaaaa

SEQ ID NO: 222 - DNA, 939 bp, synthetic construct
  source 1..939: mol_type = other DNA
aactttttcacctctgcctaatcatctcttgttcatgtcctactgttcaagcctccaagctgtgccttgggtggctttagggcatggacattgacccttataaacaatttggagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaacgcgttaagatacattgatgagtttggacaaaccacaactagaatgcagtgaaaaaaatgctttatttgtgaaatttgtgatgctattgctttatttgtaaccattataagctgcaataaacaagttaac

SEQ ID NO: 223 - DNA, 243 bp, synthetic construct
  source 1..243: mol_type = other DNA
aactttttcacctctgcctaatcatctcttgttcatgtcctactgttcaagcctccaagctgtgccttgggtggctttagggcatggacatcccttataaacaatttggataacgcgttaagatacattgatgagtttggacaaaccacaactagaatgcagtgaaaaaaatgctttatttgtgaaatttgtgatgctattgctttatttgtaaccattataagctgcaataaacaagttaac

SEQ ID NO: 224 - DNA, 239 bp, synthetic construct
  source 1..239: mol_type = other DNA
aggtttttcacctctgcctaatcatctcttgttcatgtcctactgttcaagcctccaagctgtgccttgggtggctttggggcatggacattgacacgtataaagaatttggagcttctgtggagttactctcttttttgccttctgacttctttccttctattcaagatctcatcgacaccgcctctgcttaataagacaaaccacaactagaatgcagtgaaaaaaatgctttattt

SEQ ID NO: 225 - DNA, 1323 bp, synthetic construct
  source 1..1323: mol_type = other DNA
ggtaccgtcgactgcagaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgactagtaggcaatcagcgcatgcgtggaaccattgtggctcctctgccgatccatactgcggaactcctagccgcttgttttgctcgcagccggtctggggcaaagctcatcggaactgacaattctgttgtcctctcgcggaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaacaatttggataacgatcgtaaaacta

SEQ ID NO: 226 - DNA, 2730 bp, synthetic construct
  source 1..2730: mol_type = other DNA
ggtggcacggttcactaaacgagctctgcttatatagacctcccaccgtacacgcctaccgcccatttgcgtcaatggggcggagttgttacgacattttggaaagtcccgttgattttggctgttgccctctggtttctccccaggttatccggcgggtttctgacatccggcgggtgactcacaaccccagaaacagacatatgcccctatcttatcaacacttccggaaactactgttgttagacgacgaggcaggccccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcctttcctcacattcatttacaggaggacattattaatagatgtcaacaatatgtgggccctcttacagttaatgaaaaaaggagattaaaattaattatgcctgctaggttctatcctaaccttaccaaatatttgcccttggacaaaggcattaaaccctattatcctgaacatgctgttaatcattacttcaaaactaggcattatttacatactctgtggaaggctggcattctatataagagagaaactacacgcagtgcctcattttgtgggtcaccatattcttgggaacaagagctacagcatgggaggttggtcttccaaacctcgacaaggcatttttcctgctggtggctccagttccggaacagtaaaccctgttccgactactgcctcacccatatcgtcaatcttctcgaggactggggaccctgcaccgaacatggagaacacaacatcagaattcctaggacccctgctcgtgttacaggcggggtttttcttgttgacaagaatcctcacaataccacagagtctagactcgtggtggacttctctcaattttctagggggagcacccgcgtgtcctggccaaaatttgcagtccccaacctccaatcactcaccaacctcttgtcctccgatttgacctggctatcgctggatgtgtctgcggcgttttatcatattcctcttcatcctgctgctatgcctcatcttcttgttggttcttctggactaccagggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcttgggctttcgcaagattcctatgggagtgggcctcagtccgtttctcctgactcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttaacccctaataaaaccaaacgttggggctactcccttaacttcatgggatatgtaattggaagttggggtactttaccgcaagaacatattgtacttaaaatcaaacaatgttttcgaaaactgcctgtaaatagacctattgattggaaagtatgtcagagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgccaggcaacggtcaggtctctgccaagtgtttgctgacgcaacccccactggatggggcttggctattggccatcgccgcatgcgtggaacctttgtggctcctctgccgatccatactgcggaactcctagcagcttgttttgctcgcagccggtctggagcaaaacttatcggaaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctatcgtccccttctacatctgccgttccggccgaccacggggcgcacctctctttacgcggtctccccgtctgtgccttctcatctgccggaccgtgtgcacttcgcttcacctctgcacgtcgcatggagaccaccgtgaacgccctccaggtcttgcccaaggtcttacataagaggactctcggactctcagcaatgtcaacgaccgaccttgaggcgtacttcaaagactgtttgtttaaggactgggaggagttgggggaggagattaggttaatgatctttgtactaggaggctgtaggcataaattggtctgttcaccagcaccatgcaactttttcacctctgcctaatcatctcttgttcatgtcctactgttcaagcctccaagctgtgccttgggtggctttggggcatggacatcgacccttataaagaatttggagcttcaaaaaaaaaaaaaaaaaaaaaaaaaaaaaaaaaaaaaaaaaaaaaaaaaaaaaaaaaaaaaaaaaaa

SEQ ID NO: 227 - DNA, 740 bp, synthetic construct
  source 1..740: mol_type = other DNA
tggacacacaaaaaagaagaaatagatttatttttaaatttttgtgtgcgaccatttttccaggaatcctttttgcaacgtttattctggcgagccttctgatgagcgtggtcaatgtcttaattatcattaacacgaagattattatctagtctgctatattgtaaagaaggtatttctgattttacagtcctgccttttcttttaattttatcattctgtatgaaatagtagcttactagacaaaaatgttaaggttattacacctaatctcttattttacacaaagattaaggctatcaccgtttttaaaacgacaaattaaagtaatgataattataaaaaacttaatcttaaagatcaattttgttttaagaagttgtgttatcaaccaaaacactattccatctgacaggatattgatacaacattattatatcaataacaatatgagcccagacctttcgttaaagcttaattataaagagtgcagagtttattatgttgcgttaaattcattgcggcagtaatatgtcacacatgctgcattgtgaattttttgattatcacgcagggagagaggctaaatatagtatataatatagggactcctttcgttatttaaatagttgtttattgagattagatcaatatgatgtatgagagcaatttatgagttttttataatctgtatgttaataaccaacctttgcaagtatcaggatactatgcacggtaat

SEQ ID NO: 228 - DNA, 743 bp, synthetic construct
  source 1..743: mol_type = other DNA
aggtggacacacaaaaaagaagaaatagatttatttttaaatttttgtgtgcgaccatttttccaggaatcctttttgcaacgtttattctggcgagccttctgatgagcgtggtcaatgtcttaattatcattaacacgaagattattatctagtctgctatattgtaaagaaggtatttctgattttacagtcctgccttttcttttaattttatcattctgtatgaaatagtagcttactagacaaaaatgttaaggttattacacctaatctcttattttacacaaagattaaggctatcaccgtttttaaaacgacaaattaaagtaatgataattataaaaaacttaatcttaaagatcaattttgttttaagaagttgtgttatcaaccaaaacactattccatctgacaggatattgatacaacattattatatcaataacaatatgagcccagacctttcgttaaagcttaattataaagagtgcagagtttattatgttgcgttaaattcattgcggcagtaatatgtcacacatgctgcattgtgaattttttgattatcacgcagggagagaggctaaatatagtatataatatagggactcctttcgttatttaaatagttgtttattgagattagatcaatatgatgtatgagagcaatttatgagttttttataatctgtatgttaataaccaacctttgcaagtatcaggatactatgcacggtaat

SEQ ID NO: 229 - DNA, 737 bp, synthetic construct
  source 1..737: mol_type = other DNA
tggacacacaaaaaagaagaaatagatttatttttaaatttttgtgtgcgaccatttttccaggaatcctttttgcaacgtttattctggcgagccttctgatgagcgtggtcaatgtcttaattatcattaacacgaagattattatctagtctgctatattgtaaagaaggtatttctgattttacagtcctgccttttcttttaattttatcattctgtatgaaatagtagcttactagacaaaaatgttaaggttattacacctaatctcttattttacacaaagattaaggctatcaccgtttttaaaacgacaaattaaagtaatgataattataaaaaacttaatcttaaagatcaattttgttttaagaagttgtgttatcaaccaaaacactattccatctgacaggatattgatacaacattattatatcaataacaatatgagcccagacctttcgttaaagcttaattataaagagtgcagagtttattatgttgcgttaaattcattgcggcagtaatatgtcacacatgctgcattgtgaattttttgattatcacgcagggagagaggctaaatatagtatataatatagggactcctttcgttatttaaatagttgtttattgagattagatcaatatgatgtatgagagcaatttatgagttttttataatctgtatgttaataaccaacctttgcaagtatcaggatactatgcacggt

SEQ ID NO: 230 - DNA, 737 bp, synthetic construct
  source 1..737: mol_type = other DNA
aggacacacaaaaaagaagaaatagatttatttttaaatttttgtgtgcgaccatttttccaggaatcctttttgcaacgtttattctggcgagccttctgatgagcgtggtcaatgtcttaattatcattaacacgaagattattatctagtctgctatattgtaaagaaggtatttctgattttacagtcctgccttttcttttaattttatcattctgtatgaaatagtagcttactagacaaaaatgttaaggttattacacctaatctcttattttacacaaagattaaggctatcaccgtttttaaaacgacaaattaaagtaatgataattataaaaaacttaatcttaaagatcaattttgttttaagaagttgtgttatcaaccaaaacactattccatctgacaggatattgatacaacattattatatcaataacaatatgagcccagacctttcgttaaagcttaattataaagagtgcagagtttattatgttgcgttaaattcattgcggcagtaatatgtcacacatgctgcattgtgaattttttgattatcacgcagggagagaggctaaatatagtatataatatagggactcctttcgttatttaaatagttgtttattgagattagatcaatatgatgtatgagagcaatttatgagttttttataatctgtatgttaataaccaacctttgcaagtatcaggatactatgcacggt

SEQ ID NO: 231 - DNA, 469 bp, synthetic construct
  source 1..469: mol_type = other DNA
actcggaattttgtgattccgagcaatttgtcttttaactatttccaatgttttaccccaagctttagggttgttgaatcttcaatcccctaagtgttcaagttcaaaaccaaggttaattctcaatcttgcttgtttgactgtgaactaatgctgtcctttaacaataatgtttctattaaggttgttggatcaccctgcagagtctgaggtggaacttgtgatgtgataagacctaggtgttgacaagggattttcaactgagcacactgatgcccttgctcccttcggagacctaggttgttctctatctttgtgatgtggctctgaaacaaaccaggtgtgattacagtaacaatttcaatttaaattccgatataaatttcaatgagaggaaaattattaatcttcctcatagttattcgcacacaaaagatcctaaaaattcttctttctttttgtgtgtccg

SEQ ID NO: 232 - DNA, 708 bp, synthetic construct
  source 1..708: mol_type = other DNA
tggacacacaaaaaagagaaaatacagattttttaatttatttgtgtgcgcttaattttcctggtgaggttaagatcttcctgcacctctaatgtaacttttttcttactactaattaaatcattgacattgagattaaaaacaaaaaaatgtcgtaagaacatcttatggaatttgtagattatcaatcaactacctggttggaataaaaggttcattgaccgtgttcttcttatgttattcccaccggccctaggaaatattttggctgtataagtattaaacttagctttaaaagtgaaattgcaatttagtatcagaaataacctgtcatgtgaaacatattactagttcttagcataagagtattagtcttagtaattttttcttaatacttatgtaactggagtcaacctagcttctaaatttgaatttaatatatgaactagggcaacaatttggttgtgatgttcgttatagatttgtttattttagtcataatagctaggaaaaattattatttacattatcaacataatcaatagagtgtcttcgaaaccttgaatcgatgcttccaacgctgtatattggaagagtcaataaactaatgccaaatgtggtcccttgcatattgttcttacacttgtattgactttgatatatacctggttatttaaaatacttctagaacctgagatgcagttagaa

SEQ ID NO: 233 - DNA, 711 bp, synthetic construct
  source 1..711: mol_type = other DNA
aggtggacacacaaaaaagagaaaatacagattttttaatttatttgtgtgcgcttaattttcctggtgaggttaagatcttcctgcacctctaatgtaacttttttcttactactaattaaatcattgacattgagattaaaaacaaaaaaatgtcgtaagaacatcttatggaatttgtagattatcaatcaactacctggttggaataaaaggttcattgaccgtgttcttcttatgttattcccaccggccctaggaaatattttggctgtataagtattaaacttagctttaaaagtgaaattgcaatttagtatcagaaataacctgtcatgtgaaacatattactagttcttagcataagagtattagtcttagtaattttttcttaatacttatgtaactggagtcaacctagcttctaaatttgaatttaatatatgaactagggcaacaatttggttgtgatgttcgttatagatttgtttattttagtcataatagctaggaaaaattattatttacattatcaacataatcaatagagtgtcttcgaaaccttgaatcgatgcttccaacgctgtatattggaagagtcaataaactaatgccaaatgtggtcccttgcatattgttcttacacttgtattgactttgatatatacctggttatttaaaatacttctagaacctgagatgcagttagaa

SEQ ID NO: 234 - DNA, 705 bp, synthetic construct
  source 1..705: mol_type = other DNA
tggacacacaaaaaagagaaaatacagattttttaatttatttgtgtgcgcttaattttcctggtgaggttaagatcttcctgcacctctaatgtaacttttttcttactactaattaaatcattgacattgagattaaaaacaaaaaaatgtcgtaagaacatcttatggaatttgtagattatcaatcaactacctggttggaataaaaggttcattgaccgtgttcttcttatgttattcccaccggccctaggaaatattttggctgtataagtattaaacttagctttaaaagtgaaattgcaatttagtatcagaaataacctgtcatgtgaaacatattactagttcttagcataagagtattagtcttagtaattttttcttaatacttatgtaactggagtcaacctagcttctaaatttgaatttaatatatgaactagggcaacaatttggttgtgatgttcgttatagatttgtttattttagtcataatagctaggaaaaattattatttacattatcaacataatcaatagagtgtcttcgaaaccttgaatcgatgcttccaacgctgtatattggaagagtcaataaactaatgccaaatgtggtcccttgcatattgttcttacacttgtattgactttgatatatacctggttatttaaaatacttctagaacctgagatgcagtta

SEQ ID NO: 235 - DNA, 705 bp, synthetic construct
  source 1..705: mol_type = other DNA
aggacacacaaaaaagagaaaatacagattttttaatttatttgtgtgcgcttaattttcctggtgaggttaagatcttcctgcacctctaatgtaacttttttcttactactaattaaatcattgacattgagattaaaaacaaaaaaatgtcgtaagaacatcttatggaatttgtagattatcaatcaactacctggttggaataaaaggttcattgaccgtgttcttcttatgttattcccaccggccctaggaaatattttggctgtataagtattaaacttagctttaaaagtgaaattgcaatttagtatcagaaataacctgtcatgtgaaacatattactagttcttagcataagagtattagtcttagtaattttttcttaatacttatgtaactggagtcaacctagcttctaaatttgaatttaatatatgaactagggcaacaatttggttgtgatgttcgttatagatttgtttattttagtcataatagctaggaaaaattattatttacattatcaacataatcaatagagtgtcttcgaaaccttgaatcgatgcttccaacgctgtatattggaagagtcaataaactaatgccaaatgtggtcccttgcatattgttcttacacttgtattgactttgatatatacctggttatttaaaatacttctagaacctgagatgcagtta

SEQ ID NO: 236 - DNA, 457 bp, synthetic construct
  source 1..457: mol_type = other DNA
gttgtaggctctcaggtaaacccttagtagatagcgtgtgatgaaggtaatcagtccacttagctaaaattggctagattttgtggactaaagggatacctcggggccctcctctcaccctctcttgagacttgggatagtatcggtcttactagaaaatgctaactaccaggacaaatttagtctattactctggtaaccacacttatcaagaaggttagaattgtaggaggagccaggttaggaaaaggccaagcaatacgacccacataccttcgcaacactttaggaatgcccccaaatgggggattaactagttaaattgtctccaattggtaaatgatgagatcaatctcaatgtcaatattagtttgagtttgaggaaaatgattaatcttcctcatagttattcgcacacaaaaaatcttaaaaacttttctttctttttgtgtgtccg

SEQ ID NO: 237 - DNA, 635 bp, synthetic construct
  source 1..635: mol_type = other DNA
ggacacacaaaaaagatgaagaatgttttgtgttacttatatcaaagctttttaattttttaattttatatattggtttttcttaatttacattcacatcgacattctagaaatttaactacgaagttttatcacttgtcatcagagaaaacatgacgtggtttttttgatctcaacggtaacttaattcttttcctaatgtatttaaaagaggtgcttgtagattacagcagacacttttttatatttatgatatatggtatgtcataattcaattcaactagtgtatctcagtatttccttaatttcttacaccatattagggataaagctcaaggattcacccaatgtgccaatattaatgctaattatgagcaataacatctgagtaattaacgttaaaaagttacctctggtgtgaccaaacactaatcactagttaatctgtgcgcgtaattttctcacttgtgtgaggcctaaagcgatataagattatcttgtataaaataagctgtctaatagtcttttcgttttcttatttattttattacttctcatacaatatcttagacaatgtaaccctcactcttaaatagattgtttttgtgaattaaatgaagttctctctgttgaaa

SEQ ID NO: 238 - DNA, 636 bp, synthetic construct
  source 1..636: mol_type = other DNA
aggacacacaaaaaagatgaagaatgttttgtgttacttatatcaaagctttttaattttttaattttatatattggtttttcttaatttacattcacatcgacattctagaaatttaactacgaagttttatcacttgtcatcagagaaaacatgacgtggtttttttgatctcaacggtaacttaattcttttcctaatgtatttaaaagaggtgcttgtagattacagcagacacttttttatatttatgatatatggtatgtcataattcaattcaactagtgtatctcagtatttccttaatttcttacaccatattagggataaagctcaaggattcacccaatgtgccaatattaatgctaattatgagcaataacatctgagtaattaacgttaaaaagttacctctggtgtgaccaaacactaatcactagttaatctgtgcgcgtaattttctcacttgtgtgaggcctaaagcgatataagattatcttgtataaaataagctgtctaatagtcttttcgttttcttatttattttattacttctcatacaatatcttagacaatgtaaccctcactcttaaatagattgtttttgtgaattaaatgaagttctctctgttgaaa

SEQ ID NO: 239 - DNA, 635 bp, synthetic construct
  source 1..635: mol_type = other DNA
agacacacaaaaaagatgaagaatgttttgtgttacttatatcaaagctttttaattttttaattttatatattggtttttcttaatttacattcacatcgacattctagaaatttaactacgaagttttatcacttgtcatcagagaaaacatgacgtggtttttttgatctcaacggtaacttaattcttttcctaatgtatttaaaagaggtgcttgtagattacagcagacacttttttatatttatgatatatggtatgtcataattcaattcaactagtgtatctcagtatttccttaatttcttacaccatattagggataaagctcaaggattcacccaatgtgccaatattaatgctaattatgagcaataacatctgagtaattaacgttaaaaagttacctctggtgtgaccaaacactaatcactagttaatctgtgcgcgtaattttctcacttgtgtgaggcctaaagcgatataagattatcttgtataaaataagctgtctaatagtcttttcgttttcttatttattttattacttctcatacaatatcttagacaatgtaaccctcactcttaaatagattgtttttgtgaattaaatgaagttctctctgttgaaa

SEQ ID NO: 240 - DNA, 103 bp, synthetic construct
  source 1..103: mol_type = other DNA
ctcttcaagaatattaacagactgacaagtctcaatgtcaatgttaatattcttctttatttatatgatacacaaaatcatcatctcttgtttttgtgtgtct

SEQ ID NO: 241 - DNA, 49 bp, synthetic construct
  source 1..49: mol_type = other DNA
ataaaactaaaaaactaattgtttattatttctatatctaaacaattta

SEQ ID NO: 242 - DNA, 613 bp, synthetic construct
  source 1..613: mol_type = other DNA
ggtgtcaatatataatactatatattgtcaccataagcaatcaataaggataaaaaggaaacacggacacccaaagtagttggtcccatcccgcaattgctcattacgactagatacatactggattgcatgcactagctgcagggttaaggttagccacattcaggggccggaggactcaaagtgagcacacggggctcttcacaccttgtccattatatggcttcacacccagagggtgtgcaggcagccacgcaggctagaacactgtcgccagtggggtacttctagcctcatctgccaggtctaccatggggtttggctgagcgaccaaccacgtaacttcaaaagtgttactaagctttgcgtaggcactttgcgggtaacgatctcagctgtttttgccctgttggagcatatcagctttgaccggggaaacagaagtgcttgacctttgacaatctgggagattgccgcctattggtcattttgtttaacttctaagttacaaaagggggaggggatcataaaactggcgtacaaaattaccgtaataatagtgtactactcccgttaggagatccaggtgggaacaacctggatccagttttaa

SEQ ID NO: 243 - DNA, 65 bp, synthetic construct
  source 1..65: mol_type = other DNA
ccaagtgaccaaaatttacctctaatgaaagtgactataacttgggtctcaattagagttgtcta

SEQ ID NO: 244 - DNA, 697 bp, synthetic construct
  source 1..697: mol_type = other DNA
tatgaaagttttcaaatcaaggttatcaaaatgcaaattggtaagtaagacttaggatcttttgcaattaatttgacagctaaatggtaacaatatcaaattgtcaccataagcagtcataaaagtaaaaagtgaaacacggacacccaaagtagtcggttccgccacggacttgcgcattacgacaagcaactcactggcttgtgagcacttgctccatggttaggattagccgcattcaggggccggaggactctatagtagctcaatagactcttcacaccttgttcatgtctagcgtctcatggttttcaccatgagtaggccgccaacgcagcctggaccactgtcgccagtggggtgtgtccagactcatcgacccaagctacacacgggttagtgtgctgagcgcaacgcgtcaacaatccaaaggtgatactaggtttctcgaagtactatagcggataacggataggttgttttcaaccgtgggaacagtttatacaaccacaccggggaaacagaagtgcttgctcataagagcgctggtttgcgccccacctattgagccttgatttaaaagcttctaaattacattgggaagggagataaaacaggcgtacaaaggtaccgaagtaccagagtactagccgccacgtgggcccttggagtgggaacaaccccaaagctgttttaa

SEQ ID NO: 245 - DNA, 191 bp, synthetic construct
  source 1..191: mol_type = other DNA
accaaacaaagatttggtgaatgacaagactacactcaagaataattgtgcgcaacctttttttaagacatttatttgagttcgaattcgagctctaaggagtcagagttcaacttttttctaacatagtataattaaatcaccaaggatacaattggccagaaaaggagcctattaatatgtgattttcg

SEQ ID NO: 246 - DNA, 191 bp, synthetic construct
  source 1..191: mol_type = other DNA
aggaccaaacaaagatttggtgaatgacaagactacactcaagaataattgtgcgcaacctttttttaagacatttatttgagttcgaattcgagctctaaggagtcagagttcaacttttttctaacatagtataattaaatcaccaaggatacaattggccagaaaaggagcctattaatatgtgattt

SEQ ID NO: 247 - DNA, 191 bp, synthetic construct
  source 1..191: mol_type = other DNA
agcaaacaaagatttggtgaatgacaagactacactcaagaataattgtgcgcaacctttttttaagacatttatttgagttcgaattcgagctctaaggagtcagagttcaacttttttctaacatagtataattaaatcaccaaggatacaattggccagaaaaggagcctattaatatgtgattttcg

SEQ ID NO: 248 - DNA, 191 bp, synthetic construct; identical to SEQ ID NO: 245
  source 1..191: mol_type = other DNA

SEQ ID NO: 249 - DNA, 374 bp, synthetic construct
  source 1..374: mol_type = other DNA
tttttttttttttttgaaatattaaaaacaaaataaagacataaaagaaaaatttaatatgattttgtaaattgatataaaagacagcattatgcgctgcctgtagtgtttgtaagaggcggtatattacctgtagtgtaattgtatatacggcaattatgcctgattataaagtgtgtataaaattacttaggagcagcattatgcactgcccttagtgttataatttggtaatatagcagccttatgcactgcatcggcggcattatgcaccaccgtcaccatagctcggaagtacatcgaactagggtgaatttagtgtttagtaagacagccttatacactgcctgtagtgttatgtggttgacaagatg

SEQ ID NO: 250 - DNA, 283 bp, synthetic construct
  source 1..283: mol_type = other DNA
ctctgatatttaaatagcaatataatataaataggttaggtactaatgcacaacctatttaggtcagccgtagagggttatggggtgacctcttatagctaggtgcgaaaacgccctagcattagcatggtcattgtcagtgacctgcgttgcttctatctcaaaatgtggaaagcatctttgcagtgacttgacgaatgggctgtctgcgtctaagtcaacatgaactttctccattttggataggtggaaatagggtggttgcctctacaccgtaccctat

SEQ ID NO: 251 - DNA, 475 bp, synthetic construct
  source 1..475: mol_type = other DNA
tgggccaggattctcctcgacgtcaccgcatgttagcagacttcctctgccctctccactgccagctaggtgcgaaaacgccctagcattagcatggtcattgtcagtgacctgcgttgcttctatctcaaaatgtggaaagcatctttgcagtgacttgacgaatgggctgtctgcgtctaagtcaacatgaactttctccattttggataggtggaaatagggtggttgcctctacaccgtaccctatttaggtcagccgtagagggttatggggtgacctcttatagctaggtgcgaaaacgccctagcattagcatggtcattgtcagtgacctgcgttgcttctatctcaaaatgtggaaagcatctttgcagtgacttgacgaatgggctgtctgcgtctaagtcaacatgaactttctccattttggataggtggaaatagggtggttgcctctacaccgtaccctat

SEQ ID NO: 252 - DNA, 283 bp, synthetic construct
  source 1..283: mol_type = other DNA
atagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctataagaggtcaccccataaccctctacggctgacctaaataggttgtgcattagtacctaacctatttatattatattgctatttaaatatcagag

SEQ ID NO: 253 - DNA, 475 bp, synthetic construct
  source 1..475: mol_type = other DNA
atagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctataagaggtcaccccataaccctctacggctgacctaaatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctggcagtggagagggcagaggaagtctgctaacatgcggtgacgtcgaggagaatcctggccca

SEQ ID NO: 254 - DNA, 381 bp, synthetic construct
  source 1..381: mol_type = other DNA
catcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttacaaacactacaggcagcgcataatgctgtcttttatatcaatttacaaaatcatattaaatttttcttttatgtctttattttgtttttaatatttcaaaaaaaaaaaaaaaaaaaaaa

SEQ ID NO: 255 - DNA, 135 bp, synthetic construct
  source 1..135: mol_type = other DNA
tttttttttttttttgaaatattaaaaacaaaatccgattcggaaaagaaaagaaaaaataaaataaaaattttaaggcggcatgccaatcgccgcgagttctatgtaagcagcttgccaattgctgctgtattc

SEQ ID NO: 256 - DNA, 373 bp, synthetic construct
  source 1..373: mol_type = other DNA
cttggcggactagactatgtcgtagtccattcaggttagccgtagagagttataggggcccctctcaggtagctgaatgatttaacactgctagctagagtagtcatggccataactatgatggaagttcctacggtttcataccttgattctactgccttgcacagctctgaaagaatacccactgaagccagatgcgaaaacgctctggcattagcatggtcattatcagtgacctgcttggcttctacctcaaactgcgggaagctccgctgcaaagctctgaggaatgggctgtcttcctcgatgtcaacgtgaactttctccattttgggtaggtaattggtctgggcttctctcatgcgccgcccat

SEQ ID NO: 257 - DNA, 477 bp, synthetic construct
  source 1..477: mol_type = other DNA
tgggccaggattctcctcgacgtcaccgcatgttagcagacttcctctgccctctccactgcctgaagccagatgcgaaaacgctctggcattagcatggtcattatcagtgacctgcttggcttctacctcaaactgcgggaagctccgctgcaaagctctgaggaatgggctgtcttcctcgatgtcaacgtgaactttctccattttgggtaggtaattggtctgggcttctctcatgcgccgcccattcaggttagccgtagagagttataggggcccctctcagtgaagccagatgcgaaaacgctctggcattagcatggtcattatcagtgacctgcttggcttctacctcaaactgcgggaagctccgctgcaaagctctgaggaatgggctgtcttcctcgatgtcaacgtgaactttctccattttgggtaggtaattggtctgggcttctctcatgcgccgcccat

SEQ ID NO: 258 - DNA, 373 bp, synthetic construct
  source 1..373: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 259 - DNA, 477 bp, synthetic construct
  source 1..477: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcactgagaggggcccctataactctctacggctaacctgaatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaggcagtggagagggcagaggaagtctgctaacatgcggtgacgtcgaggagaatcctggccca

SEQ ID NO: 260 - DNA, 144 bp, synthetic construct
  source 1..144: mol_type = other DNA
gaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaa

SEQ ID NO: 261 - DNA, 315 bp, synthetic construct
  source 1..315: mol_type = other DNA
tttttttttttttttgaaattttaaaaacaaaataaaaacaaaagaaaaattataaaagaatttataaagtgtgtatagtgtttttaagtaaaatatgtgtagtgtatattagagacccatatgagtctcataaaagtgatatagtgttgttaatatgtagtgtgatatacgattaatgtgacactgcacagcattatgcactgtgcttcctcggaaatacatcgagttttaatgtcaagcggcgcgtggcattatgcaccacgcttcctcggaagtacatcgagttttaccgctatgtcagtggccgcgctcag

SEQ ID NO: 262 - DNA, 266 bp, synthetic construct
  source 1..266: mol_type = other DNA
tttggcggtgggtaggtgcgtgtctactacgtcacctatttaggtcagccgtagagggtgattgggttccctcttattgccacatgcgaaaacgctctggcattggcatggtcattgtcagtgacctgccttgcttcgatctcaaattgtggaaagctccgctgtaacgacttgacatacgggctgtcagcatctaagtcaacgtgaattctttccatattggatcgattagtttgtagggtaggttcctctataccgtgccctat

SEQ ID NO: 263 - DNA, 479 bp, synthetic construct
  source 1..479: mol_type = other DNA
tgggccaggattctcctcgacgtcaccgcatgttagcagacttcctctgccctctccactgcctgccacatgcgaaaacgctctggcattggcatggtcattgtcagtgacctgccttgcttcgatctcaaattgtggaaagctccgctgtaacgacttgacatacgggctgtcagcatctaagtcaacgtgaattctttccatattggatcgattagtttgtagggtaggttcctctataccgtgccctatttaggtcagccgtagagggtgattgggttccctcttattgccacatgcgaaaacgctctggcattggcatggtcattgtcagtgacctgccttgcttcgatctcaaattgtggaaagctccgctgtaacgacttgacatacgggctgtcagcatctaagtcaacgtgaattctttccatattggatcgattagtttgtagggtaggttcctctataccgtgccctat

SEQ ID NO: 264 - DNA, 266 bp, synthetic construct
  source 1..266: mol_type = other DNA
atagggcacggtatagaggaacctaccctacaaactaatcgatccaatatggaaagaattcacgttgacttagatgctgacagcccgtatgtcaagtcgttacagcggagctttccacaatttgagatcgaagcaaggcaggtcactgacaatgaccatgccaatgccagagcgttttcgcatgtggcaataagagggaacccaatcaccctctacggctgacctaaataggtgacgtagtagacacgcacctacccaccgccaaa

SEQ ID NO: 265 - DNA, 479 bp, synthetic construct
  source 1..479: mol_type = other DNA
atagggcacggtatagaggaacctaccctacaaactaatcgatccaatatggaaagaattcacgttgacttagatgctgacagcccgtatgtcaagtcgttacagcggagctttccacaatttgagatcgaagcaaggcaggtcactgacaatgaccatgccaatgccagagcgttttcgcatgtggcaataagagggaacccaatcaccctctacggctgacctaaatagggcacggtatagaggaacctaccctacaaactaatcgatccaatatggaaagaattcacgttgacttagatgctgacagcccgtatgtcaagtcgttacagcggagctttccacaatttgagatcgaagcaaggcaggtcactgacaatgaccatgccaatgccagagcgttttcgcatgtggcaggcagtggagagggcagaggaagtctgctaacatgcggtgacgtcgaggagaatcctggccca

SEQ ID NO: 266 - DNA, 322 bp, synthetic construct
  source 1..322: mol_type = other DNA
ctgagcgcggccactgacatagcggtaaaactcgatgtacttccgaggaagcgtggtgcataatgccacgcgccgcttgacattaaaactcgatgtatttccgaggaagcacagtgcataatgctgtgcagtgtcacattaatcgtatatcacactacatattaacaacactatatcacttttatgagactcatatgggtctctaatatacactacacatattttacttaaaaacactatacacactttataaattcttttataatttttcttttgtttttattttgtttttaaaatttcaaaaaaaaaaaaaaaaaaaaaa

SEQ ID NO: 267 - DNA, 1176 bp, unidentified
  source 1..1176: mol_type = genomic DNA; note = Respiratory Syncytial Virus
atggccctgagcaaagtgaagctgaacgacaccctgaacaaggatcagctgctgagcagcagcaagtacaccatccagcggagcaccggcgacagcatcgacacccccaactacgacgtgcagaagcacatcaacaagctgtgcggcatgctgctgatcaccgaggacgccaaccacaagttcaccggcctgatcggcatgctgtacgccatgagcagactgggacgcgaggacaccatcaagatcctgcgggacgccggctaccacgtgaaggccaatggcgtggacgtgaccacccaccggcaggacatcaacggcaaagaaatgaagttcgaggtgctgaccctggccagcctgaccaccgagatccagatcaacatcgagatcgagagccggaagtcctacaagaaaatgctgaaagaaatgggcgaggtggcccccgagtaccggcacgatagccccgactgcggcatgatcatcctgtgtatcgctgccctggtgatcacaaagctggccgctggcgacagatccggactgaccgctgtgatcagacgggccaacaacgtgctgaagaacgagatgaagcggtacaagggcctgctgcccaaggatatcgccaacagcttctacgaggtgttcgagaagcacccccacttcatcgacgtgttcgtgcacttcggaatcgcccagagcagcaccagaggcggcagccgggtggaaggcatcttcgccggcctgttcatgaacgcctacggcgctggccaggtgatgctgagatggggcgtgctggccaagagcgtgaagaacatcatgctgggccacgccagcgtgcaggccgagatggaacaggtggtggaagtgtacgagtacgcccagaagctgggcggcgaggccggcttctaccacatcctgaacaaccccaaggccagcctgctgtccctgacccagttcccccacttctccagcgtggtgctgggcaatgccgccggactgggcatcatgggcgagtacagaggcaccccccggaaccaggacctgtacgacgccgccaaggcctacgccgagcagctgaaagaaaacggcgtgatcaactacagcgtgctggatctgaccgccgaggaactggaagccatcaagcaccagctgaaccccaaggacaacgacgtggaactgtga

SEQ ID NO: 268 - DNA, 381 bp, unidentified
  source 1..381: mol_type = genomic DNA; note = Respiratory Syncytial Virus
atggaaaagttcgcccccgagttccacggcgaggacgccaacaaccgggccaccaagtttctggaatccatcaagggcaagttcaccagccccaaggaccccaagaagaaggacagcatcatcagcgtgaacagcatcgacatcgaagtgaccaaagagagccccatcaccagcaacagcaccatcatcaaccccaccaacgagacagacgacaccgccggcaacaagcccaactaccagcggaagcccctggtgtccttcaaagaggaccccacccccagcgacaaccccttcagcaagctgtacaaagagacaatcgagacattcgacaacaacgaggaagagagcagctacagctacgaggaaatcaacgaccagtga

SEQ ID NO: 269 - DNA, 1269 bp, unidentified
  source 1..1269: mol_type = genomic DNA; note = SARS-CoV-1
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 270 - DNA, 564 bp, unidentified
  source 1..564: mol_type = genomic DNA; note = SARS-CoV-1
atggcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaattaa

SEQ ID NO: 271 - DNA, 1260 bp, unidentified
  source 1..1260: mol_type = genomic DNA; note = SARS-CoV-1
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccggagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 272 - DNA, 624 bp, unidentified
  source 1..624: mol_type = genomic DNA; note = SARS-CoV-1
atggcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctctgcaacctgaagaggagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaataa

SEQ ID NO: 273 - DNA, 558 bp, synthetic construct
  source 1..558: mol_type = other DNA
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 274 - DNA, 722 bp, synthetic construct
  source 1..722: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtaa

SEQ ID NO: 275 - DNA, 930 bp, synthetic construct
  source 1..930: mol_type = other DNA
atgaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccactagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagcttctgaacctgcccaagaagatcatcttcgtgggccacgactggggcgccgccctggccttccactacgcctacgagcaccaggacaggatcaaggccatcgtgcacatggagagcgtggtggacgtgatcgagagctgggacgagtggccagacatcgaggaggacatcgccctgatcaagagcgaggagggcgagaagatggtgctggagaacaacttcttcgtggagaccgtgctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagctgttcatcgagggcgaccccggcttcttcagcaacgccatcgtggagggcgccaagaagttccccaacaccgagttcgtgaaggtgaagggcctgcacttcctccaggaggacgcccccgacgagatgggcaagtacatcaagagcttcgtggagagagtgctgaagaacgagcagtaa

SEQ ID NO: 276 - DNA, 711 bp, synthetic construct
  source 1..711: mol_type = other DNA
atggtaagcaagggcgaggaggacaatatggcttcactcccggctacacatgaattgcatatatttggatcaataaacggggtggatttcgatatggtcggccaaggcaccggcaatcctaacgacggatacgaagaattgaatcttaagagtactaagggtgacttgcaattctccccgtggatattggttccccatatcggctacggattccatcaatacctcccttatcccgacggcatgagtccctttcaggccgctatggtggacggatcaggttatcaagtgcatcgaaccatgcagtttgaggatggtgcgtctctcactgttaattataggtacacatacgaaggctcccatatcaaaggtgaggcgcaagtaaaaggcacagggtttccggccgatggtccagttatgaccaatagccttacagccgcggactggtgtcggtctaaaaaaacctatccaaatgacaaaacaatcattagcacattcaagtggagctacactacgggtaacggcaagcgatatcggagcaccgccaggacaacttataccttcgcaaagcctatggcagccaactaccttaagaatcagcccatgtacgttttccgcaagaccgagcttaagcatagcaaaacagagcttaacttcaaggaatggcagaaagcgttcaccgatgtaatgggaatggacgagctgtacaaataa

SEQ ID NO: 277 - DNA, 3075 bp, synthetic construct
  source 1..3075: mol_type = other DNA
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 278 - DNA, 564 bp, Homo sapiens
  source 1..564: mol_type = genomic DNA
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 279 - DNA, 603 bp, Homo sapiens
  source 1..603: mol_type = genomic DNA
atggctgcagcttggaccgtggtgctggtgactttggtgctaggcttggccgtggcaggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctga

SEQ ID NO: 280 - DNA, 591 bp, Homo sapiens
  source 1..591: mol_type = genomic DNA
atgaccggggactgcatgccagtgctggtgctgatggccgcagtgctgaccgtgactggagcagttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 281 - DNA, 462 bp, Homo sapiens
  source 1..462: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 282 - DNA, 1710 bp, Homo sapiens
  source 1..1710: mol_type = genomic DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcatctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 283 - DNA, 534 bp, Homo sapiens
  source 1..534: mol_type = genomic DNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 284 - DNA, 702 bp, Homo sapiens
  source 1..702: mol_type = genomic DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgcttttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 285 - DNA, 873 bp, Homo sapiens
  source 1..873: mol_type = genomic DNA
atgattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcacgatgaagttgatgggggatcccccatggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataagagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagactga

SEQ ID NO: 286 - DNA, 1050 bp, Homo sapiens
  source 1..1050: mol_type = genomic DNA
atggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatccaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttttattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctag

SEQ ID NO: 287 - DNA, 549 bp, Homo sapiens
  source 1..549: mol_type = genomic DNA
atgaacaacaggtggatcctccacgctgcattcctgctgtgcttctccaccacagccctctccatcaactataagcagctccagctccaagaaaggacaaacattcggaaatgtcaggagctcctggagcagctgaatggaaagatcaacctcacctacagggctgacttcaagatccctatggagatgacagagaagatgcagaagtcttacactgcctttgccatccaagagatgctccagaatgtctttcttgtcttcagaaacaatttctccagcactgggtggaatgagactattgttgtaaggctcctggatgaactccaccagcagacagtgtttctgaagacagtattggaggaaaagcaagaggaaagattgacgtgggagatgtcctctactgctctccacttgaagagctattactggagggtgcaaaggtacctcaaactcatgaagtacaacagctacgcctggatggtggtccgggcagagatcttcaggaactttctcatcattcgaagacttaccagaaacttccaaaactga

SEQ ID NO: 288 - DNA, 582 bp, Mus musculus
  source 1..582: mol_type = genomic DNA
atgctcctcctgctgttgcctctgctgctggccgcagtgctgacaagaacccaagctgaccctgtccccagggccaccaggctcccagtggaagcaaaggattgccacattgctcagttcaagtctctgtccccaaaagagctgcaggccttcaaaaaggccaaggatgccatcgagaagaggctgcttgagaaggacctgaggtgcagttcccacctcttccccagggcctgggacctgaagcagctgcaggtccaagagcgccccaaggccttgcaggctgaggtggccctgaccctgaaggtctgggagaacatgactgactcagccctggccaccatcctgggccagcctcttcatacactgagccacattcactcccagctgcagacctgtacacagcttcaggccacagcagagcccaggtccccgagccgccgcctctcccgctggctgcacaggctccaggaggcccagagcaaggagacccctggctgcctggaggcctctgtcacctccaacctgtttcgcctgctcacccgggacctcaagtgtgtggccaatggagaccagtgtgtctga

SEQ ID NO: 289 - DNA, 582 bp, Mus musculus
  source 1..582: mol_type = genomic DNA
atgctcctcctgctgttgcctctgctgctggccgcagtgctgacaagaacccaagctgaccctgtccccagggccaccaggctcccagtggaagcaaaggattgccacattgctcagttcaagtctctgtccccaaaagagctgcaggccttcaaaaaggccaagggtgccatcgagaagaggctgcttgagaaggacatgaggtgcagttcccacctcatctccagggcctgggacctgaagcagctgcaggtccaagagcgccccaaggccttgcaggctgaggtggccctgaccctgaaggtctgggagaacataaatgactcagccctgaccaccatcctgggccagcctcttcatacactgagccacattcactcccagctgcagacctgtacacagcttcaggccacagcagagcccaagcccccgagtcgccgcctctcccgctggctgcacaggctccaggaggcccagagcaaggagactcctggctgcctggaggactctgtcacctccaacctgtttcaactgctcctccgggacctcaagtgtgtggccagtggagaccagtgtgtctga

SEQ ID NO: 290 - DNA, 600 bp, Mus musculus
  source 1..600: mol_type = genomic DNA
atgactccaaagtttttatggctggtggcccttgtggctctatacattccgcccatccaatctctgaactgtgtttacctggatgatagcatcttggaaaatgtgaaacttctgggcagtaccatgaccggctttcccttaagatgtctaaaagatatcacagattttaagtttcctaaagagattttgccatacatccagcatatgaaaagggagataaacgccgtctcctatcgtatatcctctctggcactaactatcttcaatcttaaaggctccatccctccagtgacagaggaacactgggaacgtatcagatcgggacttttcaaacaagtgcggcaagctcaagagtgcttcatggacgaggagaaagagaacagggaacatcctcactccgaggacttcctgacagtctacctggagttgggcaagtatttcttcagaatcaaaaagttcctgataaataagaaatacagtttctgtgcatggaagattgtcacagtggaaataagaagatgtttcattatattttccaagtccagaaaactactcaaaatgatatcagaatcacccaccttcaagcaagaacttaaatag

SEQ ID NO: 291 - DNA, 510 bp, Mus musculus
  source 1..510: mol_type = genomic DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 292 - DNA, 1782 bp, Mus musculus
  source 1..1782: mol_type = genomic DNA
atggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaagcggagctactaacttcagcctgctgaagcaggctggagacgtggaggagaaccctggacctatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctga

SEQ ID NO: 293 - DNA, 543 bp, Mesocricetus auratus
  source 1..543: mol_type = genomic DNA
atgaccaacaggtggatccttcaagctgcatttctgctgtggtttctccccatatccctgtccatcaactacgaggaacttgaggccagacaaagcagaagcaattcagagtgtgaggacctcctgaaacagctgcgtggacctctctgcctcagagacagaaagttcttcaagatgcctatggaggtgaaccacccagaacagatggagaagagagacactgccttcatcatccaagagatgctccagaatatctttcttgtcttcaacaacactttctccagcactgggtggaataagaccatcgttgaaagtttcttaggtaaacttcacaatcagatagtctttctgaatgaaaccctaaaggaagtgccagaggatgaaagtttgactacaaggattagcttgaaaagctattactggcagctgggaaggtaccttaaagagaagaggtacagcagctgcgcctggatggtgacccgagcagcagtctcaaggaactttttaatcattagaaggctcacttggatcttccagagctga

SEQ ID NO: 294 - DNA, 639 bp, Mesocricetus auratus
  source 1..639: mol_type = genomic DNA
atgcccagctcctcacagaccccagagagcagcatgaagccagctgggggccgcctgctcctcctgctgctgctgctgctgctgctggccgcggtgctgacgggagcccgcgctcatcctgtgcccagggccaccaggctcccaccagatgcaaaggattgtcacatagcccggttcaagtctctgcccccgcaagagctgcaggccttcaagaaggccaaggatgccatagaagagtggctgctcaagaaggacgtcaggtgcagctcccgcctcttccccagggcctgggacctgaagcagctgcaggtccaggagcgccccgaggccttgcaggctgagctgaccctcacactgaaggtcctggagaacgtgactgacccgggcctgggccccatcctggaccagcctcttctcaccctgagccacatccactcacagctgcaggcctgtactcagcctcagcctgcagcagagcccaggccccccagccgccgcctctcccgctggctgcacaggctccaggaggcccaggagaaggagacccccggctgcctggaagtctctgtcacttccaacctcttccgcctgctcatccgggacctgaagtgtgtggccagtggagaccagtgtgactga

SEQ ID NO: 295 - DNA, 561 bp, Mustela putorius
  source 1..561: mol_type = genomic DNA
atgaccagcaggtgtatcctccaaactgctctcctggtatatttctccaccacggttcttgccatgaactataacttacttcgattccaactaagcagcagcagtgtggagtgccaggagctccttctacagttgaatggaacatctaaagactgcctcaaggacaggatgaacttcaagatccctgaggagatccagaaatcccagaagttccagaaggaggacatcgtattggtcacccttgagatgttccaaaagacttctgatattttcaggagaaatctctcaagcacgggatggaatgagagcattgtcgagaacctccttgccacactccactggcagaaggaacatctggaggaaatcctggaggacatcatgcaggaggaaaacgtcacttgggaccacaggacccttctgcacctgaagagatattatctaaggattgtgcggtacctgaaagcgaaggagttcagcgtctgtgcctggacaatagtgcaagcagaaatcctcaggagctttttcttccttgataaacttacagattctctccctaactga

SEQ ID NO: 296 - DNA, 573 bp, Sigmodon hispidus
  source 1..573: mol_type = genomic DNA
atgtccaggtcatgtgctttcctgacatttctgctggtgatgagctactggacaacctgctctctgggatgtgacctgcctcagattcataacctcaggaacaacacagccttcacactcctggtgcaaatgaggacactcttccctctctcctgcctgaaggacagaaaggactttgcatcccctctggagaaggtggttgcccagcagctccagaaggctcaagccatccttgtctcaagtgaggtgatccaacagatcctgaacattttcacctcaaatgaatcatctgctgcttgggagacaaccctcctggaaacattctgtgacagcctctaccagaagctcagagacctacaagactgtctgatgcagcaggaggaggtgaaggaacctaccctgagcgaggaagactcgctggggactgtgaggcagtacttccacaggatcactgtctacctgaaagagaggaaacacagcccctgtgcctgggaagtggtcagagcagaagtctggagagccctgtcttcctcagtcaaattgctggcaagatggagtgaggagaagaagtaa

SEQ ID NO: 297 - DNA, 546 bp, Sigmodon hispidus
  source 1..546: mol_type = genomic DNA
atggtcaacagatggattctccaacctgcattcctgctgtggtttctccctacagccctctccatcaactacaagcagcttgagctcagacaaaggagcagcatttcagaatgtctagagctcttgaatcagatgaatggaccgctctgtctcagctatagacagaacttcaagattcctatggaagtgaagcatccaagacagctagagaaggatgaagctgtctccgtcattgaagtgatgctccagaatatcttccacatcttccgcagcaattcctccagcaccgggtggaataaaaacatcattgaaagcttcttggataaactccgtaacgagatggactttctagaggaaataccagagaatgaaagtttgactccaaggaactttaccaccattctacccttgaaaagctattataagaggatgcaagagtatcttgaaaaccatgaatacagcagctgttcctggatagtagtccgaacagaaatccgcaggaacttttccatcatgggaagacttattaagatcttccagaactga

SEQ ID NO: 298 - DNA, 603 bp, Sigmodon hispidus
  source 1..603: mol_type = genomic DNA
atgaagccagaaagagctgggggccacgggctcctcctcctgctgctggccccggtgctggcgggaacccgcgctaaccctgtgcccagggccaccaggctgccaccagatacaaagcattgccacctagcccagttcaagtccctgtccccgcaagcgctgcagaacttcaggaaggccaaggatgccatagaagagtggcgtctcaagaaggaggacatcaggtgcagctcccgcctcttccccagggcgtgggacctgcagcagctgcaggtccaggagcgccccatggccctgaaggctgagctggccctgacactgagggtcctggagaacatgactgactcagccctggatcccatcctggaccagccccttcgcacactgcgccacatccaatcacagctgcaggcctgtactcagcctcagctcacagcagagcccaggcccccaagtcgccgcctctctcgctggctgcaaaggctgcaggaagcccagaggaaggagacccccggcagcctggaggcctgtgtcacatacaacctcttccgcctgctgactcgggacctgaagtgtgtggccagtggagaccagtgtgcctaa

SEQ ID NO: 299 - DNA, 543 bp, Homo sapiens
  source 1..543: mol_type = genomic DNA
atggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtga

SEQ ID NO: 300 - DNA, 336 bp, unidentified
  source 1..336: mol_type = genomic DNA; note = SARS-CoV-2
atgtttgtttttcttgttttattgccactagtctctagtgttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattaaaatacacataa

SEQ ID NO: 301 - DNA, 558 bp, synthetic construct
  source 1..558: mol_type = other DNA
ttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccaagggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccaactctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccat

SEQ ID NO: 302 - DNA, 722 bp, synthetic construct
  source 1..722: mol_type = other DNA
ttacacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccat

SEQ ID NO: 303 - DNA, 930 bp, synthetic construct
  source 1..930: mol_type = other DNA
ttactgctcgttcttcagcactctctccacgaagctcttgatgtacttgcccatctcgtcgggggcgtcctcctggaggaagtgcaggcccttcaccttcacgaactcggtgttggggaacttcttggcgccctccacgatggcgttgctgaagaagccggggtcgccctcgatgaacagcttgggcaggtcgtcgctggctctcaggtaggcgttgtagtttctcacgatctgcaccacgtcgggcttgccgcccttcaccagggggatctctctgggccagctcagggtgggtcttctcacctcgcccttctccttgaagggctccaggtaggcggcgaactcctcgggctccagctttctcatgatcttgctgggcagcacggtctccacgaagaagttgttctccagcaccatcttctcgccctcctcgctcttgatcagggcgatgtcctcctcgatgtctggccactcgtcccagctctcgatcacgtccaccacgctctccatgtgcacgatggccttgatcctgtcctggtgctcgtaggcgtagtggaaggccagggcggcgccccagtcgtggcccacgaagatgatcttcttgggcaggttcagaagctcgaaccaggcggtcaggtacttgtagtggtccagcagcctgtagctgccgttgccgctcttgccgctcttgcccatgccgatcagatcggggatgatgcacctggccacgggctcgatgtggggcaccacgtgcctccacaggtagctgctagtggcgttgccgtgcaggaagatcacggcgttctcggcgtgcttctcgctgtcgtagtagttgatgaagctgtccagcacgttcatctgcttgcacctggcccaccactgggggccggtgatcatcctcttcctctgctcggggtcgtacaccttcat

SEQ ID NO: 304 - DNA, 711 bp, synthetic construct
  source 1..711: mol_type = other DNA
ttatttgtacagctcgtccattcccattacatcggtgaacgctttctgccattccttgaagttaagctctgttttgctatgcttaagctcggtcttgcggaaaacgtacatgggctgattcttaaggtagttggctgccataggctttgcgaaggtataagttgtcctggcggtgctccgatatcgcttgccgttacccgtagtgtagctccacttgaatgtgctaatgattgttttgtcatttggataggtttttttagaccgacaccagtccgcggctgtaaggctattggtcataactggaccatcggccggaaaccctgtgccttttacttgcgcctcacctttgatatgggagccttcgtatgtgtacctataattaacagtgagagacgcaccatcctcaaactgcatggttcgatgcacttgataacctgatccgtccaccatagcggcctgaaagggactcatgccgtcgggataagggaggtattgatggaatccgtagccgatatggggaaccaatatccacggggagaattgcaagtcacccttagtactcttaagattcaattcttcgtatccgtcgttaggattgccggtgccttggccgaccatatcgaaatccaccccgtttattgatccaaatatatgcaattcatgtgtagccgggagtgaagccatattgtcctcctcgcccttgcttaccat

SEQ ID NO: 305 - DNA, 3075 bp, synthetic construct
  source 1..3075: mol_type = other DNA
ttatttttgacaccagaccaactggtaatggtagcgaccggcgctcagctggaattccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccagcgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggtcacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatccgtaatcatggtcat

SEQ ID NO: 306 - DNA, 564 bp, Homo sapiens
  source 1..564: mol_type = genomic DNA
tcagtttcggaggtaacctgtaagtctgttaatgaagtaaaagttccttaggatttccactctgactatggtccaggcacagtgactgtactccttggccttcaggtaatgcagaatcctcccataatatcttttcaggtgcagactgctcatgagttttcccctggtgaaatcttctttctccagtttttcttccaggactgtcttcagatggtttatctgatgatagacattagccaggaggttctcaacaatagtctcattccagccagtgctagatgaatcttgtctgaaaatagcaaagatgttctggagcatctcatagatggtcaatgcggcgtcctccttctggaactgctgcagctgcttaatctcctcagggatgtcaaagttcatcctgtccttgaggcagtattcaagcctcccattcaattgccacaggagcttctgacactgaaaattgctgcttctttgtaggaatccaagcaagttgtagctcatggaaagagctgtagtggagaagcacaacaggagagcaatttggaggagacacttgttggtcat

SEQ ID NO: 307 - DNA, 603 bp, Homo sapiens
  source 1..603: mol_type = genomic DNA
tcaggtggactcagggtgggttgacgttctcagacacaggttcccatcggccacatatttgaggtctcgcgtgaggaggcggaagaggttgaaggtgacagatgcctccaggcagccagcggactcctttttgggggcctcctggagccggtgcagccagtggtggaggcggccccggggcctgggccctgctgtgggctgaggctggatacaggcctggagctgggagaggatgtggtgcagggtgtgaaggggctggtctaggacgtcctccagggctgggccagcagcggcctccaggaccttcagcgtcagggccagctcagcctccaaggccacagggcgctccctcacctggagaagcctcaggtcccaattcccggggaagacaggagagctgcaactccagtttttcagcttgagtgactcttccaaggcgtccctggccttcttgaagctcgctagctcctgtggtgacagagatttgaacctgccaatgtggcagcccttcccagttgtggtgggcttggaagtggggacagggcctgccacggccaagcctagcaccaaagtcaccagcaccacggtccaagctgcagccat

SEQ ID NO: 308 - DNA, 591 bp, Homo sapiens
  source 1..591: mol_type = genomic DNA
tcagacacacaggtccccgctggcaacacaattcaggtctcgcgtgaggaggcggaagaggttgaaggtgacagaggcctcgaggcagccaggggactccttttttggggcctcctggagccggtgcagccaatggtggaggcggccccgggtcctgggccctgccgtgggctgaggctggatacaggcccggagctgggagaggatatggtgcagggtgtgaaggggctggtccaagacatcccccagggctgggtcagtgtcagcggtggcctccagaaccttcagcgtcagggccagctcagcctccaaagccacggggcgctccctcacctgcagctgcctcaggtcccaggtcctggggaagaggcgggagcggcacttgcagtccttcagcagaagcgactcttctaaggcatctttggccctcttaaaggcctgcagctcctgtggagacagggacttgaactgggctatgtggcagccccttgcatccgggagagccccgcggagcctggcgacaggaactgctccagtcacggtcagcactgcggccatcagcaccagcactggcatgcagtccccggtcat

SEQ ID NO: 309 - DNA, 462 bp, Homo sapiens
  source 1..462: mol_type = genomic DNA
tcaagtcagtgttgagatgatgctttgacaaaaggtaatccatctgttcagaaattctacaatggttgctgtctcatcagcatattcacacatgaatgttgtttcagatccctttagttccagaactattacgttgatattgctgattaagtccctgggtcttaagtgaaagtttttgctttgagctaaatttagcacttcctccagaggtttgagttcttcttctagacactgaagatgtttcagttctgtggccttcttgggcatgtaaaacttaaatgtgagcatcctggtgagtttgggattcttgtaattattaattccattcaaaatcatctgtaaatccagcagtaaatgctccagttgtagctgtgttttctttgtagaacttgaagtaggtgcactgtttgtgacaagtgcaagacttagtgcaatgcaagacaggagttgcatcctgtacat

SEQ ID NO: 310 - DNA, 1710 bp, Homo sapiens
  source 1..1710: mol_type = genomic DNA
ctaactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagatgagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacataggtccagggttctcctccacgtctccagcctgcttcagcaggctgaagttagtagctccgcttccggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcaagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctggccaaactgaggtggtccaggaggaccagggtagccacaaggaggaggctgcgcgctggacacat

SEQ ID NO: 311 - DNA, 534 bp, Homo sapiens
  source 1..534: mol_type = genomic DNA
ctactcgtcctcctggaagtagaatttggtgaccatgacgccttcgtcaggcatattggtgaggctgacgggctggtcagcttccatcgctgtgcagaggaaccaaccggggcaggcggcagactcaaaactggtggtggggccactgtctgagcggatgaaggcgaagcgcttgtcctgctttctgttctcgctcaggtcagtgatgttaactgcctccagctggagtctggtctcatcaccagacttgacacaggacaggcacatcttccctccatggattcccaagaacagagcatgaggctcaatgggtaccacatctatcttttcttctaaattgacatttggtccttgcaagtatccagcaactagttggttgttcctcagatagaaggtcttctggttaacatcccagattctgaaggcttgcatcttgctggattttctcccagagggtcggcagatcgtctctgaatggaacaggaagaggaggagagtgattaggtgactgcggaggcctctgcagatttccat

SEQ ID NO: 312 - DNA, 702 bp, Homo sapiens
  source 1..702: mol_type = genomic DNA
tcacagggcaatgatcccaaagtagacctgcccagactcggcaaagtcgagatagtcgggccgattgatctcagcgctgagtcggtcacccttctccagctggaagacccctcccagatagatgggctcataccagggcttggcctcagccccctctggggtctccctctggcaggggctcttgatggcagagaggaggttgaccttggtctggtaggagacggcgatgcggctgatggtgtgggtgaggagcacatgggtggaggggcagccttggcccttgaagaggacctgggagtagatgaggtacaggccctctgatggcaccaccagctggttatctctcagctccacgccattggccaggagggcattggcccggcggttcagccactggagctgcccctcagcttgagggtttgctacaacatgggctacaggcttgtcactcggggttcgagaagatgatctgactgcctgggccagagggctgattagagagaggtccctggggaactcttccctctgggggccgatcactccaaagtgcagcaggcagaaaagcgtggtggcgcctgccacgatcaggaaggagaagaggctgaggaacaagcaccgcctggagccctggggcccccctgtcttcttggggagcgcctcctcggccagctccacgtcccggatcatgctttcagtgctcat

SEQ ID NO: 313 - DNA, 873 bp, Homo sapiens
  source 1..873: mol_type = genomic DNA
tcagtctgtctcaatgccacagtccagttctgtaccacggcaggcctgaataatgaaaagtttgggttttccagttagacttctacaacgatcccctctgaaaaagtttgttatttttttcaggtcaacaggtccatttgttccaaaaattattccttcttcaccatggctcagaagcacacaaacaaaactgctccttttgctgtgatcttctttagaaacatcacgcatcaattccacaatttcttcacgtgtaagatcatttttattcctgacttcatatttcaagtttctgaatgtttccctgaggtttgctgcatcgacatctgtaccagaccgagatgtcattccagtgctcttatgaaaattcttattattaattattatacataaacccatctcaggataatccattttataactgttgtccagggatattccagagtccattgattcgcttccatgtatgatctttggttccaaatttttaatggattttgaatccactgagttttcagtgttctccatgggggatcccccatcaacttcatcgtgataaaaatagagttcttttgtgagcatggaaacaatacatggaatctgtttctttgcatgaaaagtagcgtcaaaggaaaaggactcaaattctgttgccacctttcggttaacccgggtaagaatgtgcataaattcaagcttgtcggcatactgtttcagcatggcacaaagcgactggatgaaccaggagccatcctttgaatttcgccaagaataataaccaggtgctgtggagtatgcatacaagaagtcggcctccactggtattttatgacacgccatgtcatcatcaacaccactgtctgtctcaatcat

SEQ ID NO: 314 - DNA, 1050 bp, Homo sapiens
  source 1..1050: mol_type = genomic DNA
ctaggctgtgcaaggggagcagtcctcccctttgtaggtgccatttcccagagctcctggaattgtttcttcagttgtggcagctgtgtagatcatttttgttttatccttgggagtcttgaatttctcatagcaccaaaatatggcaatgaggagaagcccttctccaagctggaaaatcatgtagaggaggggaaagaagaaaagtggtccaatgacttcaggtggaaaggccacattgaggatggtggaacagagttggacattttggcatccagtctccatgctgacagtgcgtctgcaccgtccattgaggcagaagagagcagagagaacataacccagcagaaagccaataaaaggcatcagggaggaggtggcaatcaagagtggtgtcatggcaaacatgatgctcttccccacattgatggcagagagaactgtgacggccacactgcacaagagaatgatgatcatccctcccttgatgacatagcgcatgtattgtggccgtttggatttgaggacgatccctatggtgcaaggaatgagaaccaggaccagtgatatcacgatgcctttatagggcaccttgtccttcaggtccccatcatagatccccctggagtagatgtacaggaggagaggcatcatgccaagggcacagaaggtggagcaggtggtcatcacaatgctgaggttcatgtcccccttcatggccagactgaagacattggacaggttccctccaggtgagcagccacagaccaagatggccagtgcctcaatgttcttcagccggaagaccttgcccagcacaaaggccgtgaggggcatgatgccatactgtgccaccagggcgatggccagccctttaggcttccataagtgagccttgatcttgctgaactccatggtgcagcccagcgagagcatgatgaagaacaacatgaacaccaggatgacgctcagtgccaggtctgtggggcgcttgccaaagttgggtggcagggtgaagttgaatggggcagacgcgttgtgggcctccat

SEQ ID NO: 315 - DNA, 549 bp, Homo sapiens
  source 1..549: mol_type = genomic DNA
tcagttttggaagtttctggtaagtcttcgaatgatgagaaagttcctgaagatctctgcccggaccaccatccaggcgtagctgttgtacttcatgagtttgaggtacctttgcaccctccagtaatagctcttcaagtggagagcagtagaggacatctcccacgtcaatctttcctcttgcttttcctccaatactgtcttcagaaacactgtctgctggtggagttcatccaggagccttacaacaatagtctcattccacccagtgctggagaaattgtttctgaagacaagaaagacattctggagcatctcttggatggcaaaggcagtgtaagacttctgcatcttctctgtcatctccatagggatcttgaagtcagccctgtaggtgaggttgatctttccattcagctgctccaggagctcctgacatttccgaatgtttgtcctttcttggagctggagctgcttatagttgatggagagggctgtggtggagaagcacagcaggaatgcagcgtggaggatccacctgttgttcat

SEQ ID NO: 316 - DNA, 582 bp, Mus musculus
  source 1..582: mol_type = genomic DNA
tcagacacactggtctccattggccacacacttgaggtcccgggtgagcaggcgaaacaggttggaggtgacagaggcctccaggcagccaggggtctccttgctctgggcctcctggagcctgtgcagccagcgggagaggcggcggctcggggacctgggctctgctgtggcctgaagctgtgtacaggtctgcagctgggagtgaatgtggctcagtgtatgaagaggctggcccaggatggtggccagggctgagtcagtcatgttctcccagaccttcagggtcagggccacctcagcctgcaaggccttggggcgctcttggacctgcagctgcttcaggtcccaggccctggggaagaggtgggaactgcacctcaggtccttctcaagcagcctcttctcgatggcatccttggcctttttgaaggcctgcagctcttttggggacagagacttgaactgagcaatgtggcaatcctttgcttccactgggagcctggtggccctggggacagggtcagcttgggttcttgtcagcactgcggccagcagcagaggcaacagcaggaggagcat

SEQ ID NO: 317 - DNA, 582 bp, Mus musculus
  source 1..582: mol_type = genomic DNA
tcagacacactggtctccactggccacacacttgaggtcccggaggagcagttgaaacaggttggaggtgacagagtcctccaggcagccaggagtctccttgctctgggcctcctggagcctgtgcagccagcgggagaggcggcgactcgggggcttgggctctgctgtggcctgaagctgtgtacaggtctgcagctgggagtgaatgtggctcagtgtatgaagaggctggcccaggatggtggtcagggctgagtcatttatgttctcccagaccttcagggtcagggccacctcagcctgcaaggccttggggcgctcttggacctgcagctgcttcaggtcccaggccctggagatgaggtgggaactgcacctcatgtccttctcaagcagcctcttctcgatggcacccttggcctttttgaaggcctgcagctcttttggggacagagacttgaactgagcaatgtggcaatcctttgcttccactgggagcctggtggccctggggacagggtcagcttgggttcttgtcagcactgcggccagcagcagaggcaacagcaggaggagcat

SEQ ID NO: 318 - DNA, 600 bp, Mus musculus
  source 1..600: mol_type = genomic DNA
ctatttaagttcttgcttgaaggtgggtgattctgatatcattttgagtagttttctggacttggaaaatataatgaaacatcttcttatttccactgtgacaatcttccatgcacagaaactgtatttcttatttatcaggaactttttgattctgaagaaatacttgcccaactccaggtagactgtcaggaagtcctcggagtgaggatgttccctgttctctttctcctcgtccatgaagcactcttgagcttgccgcacttgtttgaaaagtcccgatctgatacgttcccagtgttcctctgtcactggagggatggagcctttaagattgaagatagttagtgccagagaggatatacgataggagacggcgtttatctcccttttcatatgctggatgtatggcaaaatctctttaggaaacttaaaatctgtgatatcttttagacatcttaagggaaagccggtcatggtactgcccagaagtttcacattttccaagatgctatcatccaggtaaacacagttcagagattggatgggcggaatgtatagagccacaagggccaccagccataaaaactttggagtcat

SEQ ID NO: 319 - DNA, 510 bp, Mus musculus
  source 1..510: mol_type = genomic DNA
ttattgagggcttgttgagatgatgctttgacagaaggctatccatctcctcagaaagtccaccacagttgctgactcatcatcgaattggcactcaaatgtgttgtcagagccctttagttttacaacagttactctgatattgctgatgaaattctcagcatcttccaattgaaagcttttgctttgagtcaaatccagaacatgccgcagaggtccaagttcatcttctaggcactgaagatctttcaattctgtggcctgcttgggcaagtaaaatttgaaggtgagcatcctggggagtttcaggttcctgtaattctccatcctgctcaggagctcctgtaggtccatcaacagctgctccaggtgctgctgctgctgctgctgctgctgctgctgctgctgtgcttccgctgtagagcttgaagtggagcttgaagtgggtgcgctgttgacaaggagcacaagtgtcaatgtgacacaggatgcgagctgcatgctgtacat

SEQ ID NO: 320 - DNA, 1782 bp, Mus musculus
  source 1..1782: mol_type = genomic DNA
tcaggatcggaccctgcagggaacacatgcccacttgctgcatgaggaattgtaatagcgatcctgagcttgcacgcagacattcccgcctttgcattggacttcggtagatgtcttctctacgaggaacgcacctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacataggtccagggttctcctccacgtctccagcctgcttcagcaggctgaagttagtagctccgcttccggcggagctcagatagcccatcaccctgttgatggtcacgacgcgggtgctgaaggcgtgaagcaggatgcagagcttcattttcactctgtaagggtctgcttctcccacaggaggtttctggcgcagagtctcgccattatgattcagagactgcatcagctcatcgatggccaccagcatgcccttgtctagaatgatctgctgatggttgtgattctgaagtgctgcgttgatggcctggaactctgtctggtacatcttcaagtcctcatagatgctaccaaggcacagggtcatcatcaaagacgtcttctgtgggggcaggcagctccctcttgttgtggaagaagtctctctagtagccaggcaactctcgttcttgtgtagttccagtggtaaacaggtcttcaatgtgctggtttggtcccgtgtgatgtcttcatgatcgatgtcttcagcagtgcaggaataatgtttcagtttttctctggccgtcttcaccatgtcatctgtggtcttcagcaggtttcgggactggctaagacacctggcaggtccagagactggaatgaccctggccaaactgaggtggtttaggagggcaagggtggccaaaaagaggaggtagcgtgattgacacatgctggaccggcactgagaggagctgctggatgccgagggtgaggctgttggaacgctgaccat

SEQ ID NO: 321 - DNA, 543 bp, Mesocricetus auratus
  source 1..543: mol_type = genomic DNA
tcagctctggaagatccaagtgagccttctaatgattaaaaagttccttgagactgctgctcgggtcaccatccaggcgcagctgctgtacctcttctctttaaggtaccttcccagctgccagtaatagcttttcaagctaatccttgtagtcaaactttcatcctctggcacttcctttagggtttcattcagaaagactatctgattgtgaagtttacctaagaaactttcaacgatggtcttattccacccagtgctggagaaagtgttgttgaagacaagaaagatattctggagcatctcttggatgatgaaggcagtgtctctcttctccatctgttctgggtggttcacctccataggcatcttgaagaactttctgtctctgaggcagagaggtccacgcagctgtttcaggaggtcctcacactctgaattgcttctgctttgtctggcctcaagttcctcgtagttgatggacagggatatggggagaaaccacagcagaaatgcagcttgaaggatccacctgttggtcat

SEQ ID NO: 322 - DNA, 639 bp, Mesocricetus auratus
  source 1..639: mol_type = genomic DNA
tcagtcacactggtctccactggccacacacttcaggtcccggatgagcaggcggaagaggttggaagtgacagagacttccaggcagccgggggtctccttctcctgggcctcctggagcctgtgcagccagcgggagaggcggcggctggggggcctgggctctgctgcaggctgaggctgagtacaggcctgcagctgtgagtggatgtggctcagggtgagaagaggctggtccaggatggggcccaggcccgggtcagtcacgttctccaggaccttcagtgtgagggtcagctcagcctgcaaggcctcggggcgctcctggacctgcagctgcttcaggtcccaggccctggggaagaggcgggagctgcacctgacgtccttcttgagcagccactcttctatggcatccttggccttcttgaaggcctgcagctcttgcgggggcagagacttgaaccgggctatgtgacaatcctttgcatctggtgggagcctggtggccctgggcacaggatgagcgcgggctcccgtcagcaccgcggccagcagcagcagcagcagcagcaggaggagcaggcggcccccagctggcttcatgctgctctctggggtctgtgaggagctgggcat

SEQ ID NO: 323 - DNA, 561 bp, Mustela putorius
  source 1..561: mol_type = genomic DNA
tcagttagggagagaatctgtaagtttatcaaggaagaaaaagctcctgaggatttctgcttgcactattgtccaggcacagacgctgaactccttcgctttcaggtaccgcacaatccttagataatatctcttcaggtgcagaagggtcctgtggtcccaagtgacgttttcctcctgcatgatgtcctccaggatttcctccagatgttccttctgccagtggagtgtggcaaggaggttctcgacaatgctctcattccatcccgtgcttgagagatttctcctgaaaatatcagaagtcttttggaacatctcaagggtgaccaatacgatgtcctccttctggaacttctgggatttctggatctcctcagggatcttgaagttcatcctgtccttgaggcagtctttagatgttccattcaactgtagaaggagctcctggcactccacactgctgctgcttagttggaatcgaagtaagttatagttcatggcaagaaccgtggtggagaaatataccaggagagcagtttggaggatacacctgctggtcat

SEQ ID NO: 324 - DNA, 573 bp, Sigmodon hispidus
  source 1..573: mol_type = genomic DNA
ttacttcttctcctcactccatcttgccagcaatttgactgaggaagacagggctctccagacttctgctctgaccacttcccaggcacaggggctgtgtttcctctctttcaggtagacagtgatcctgtggaagtactgcctcacagtccccagcgagtcttcctcgctcagggtaggttccttcacctcctcctgctgcatcagacagtcttgtaggtctctgagcttctggtagaggctgtcacagaatgtttccaggagggttgtctcccaagcagcagatgattcatttgaggtgaaaatgttcaggatctgttggatcacctcacttgagacaaggatggcttgagccttctggagctgctgggcaaccaccttctccagaggggatgcaaagtcctttctgtccttcaggcaggagagagggaagagtgtcctcatttgcaccaggagtgtgaaggctgtgttgttcctgaggttatgaatctgaggcaggtcacatcccagagagcaggttgtccagtagctcatcaccagcagaaatgtcaggaaagcacatgacctggacat

SEQ ID NO: 325 - DNA, 546 bp, Sigmodon hispidus
  source 1..546: mol_type = genomic DNA
tcagttctggaagatcttaataagtcttcccatgatggaaaagttcctgcggatttctgttcggactactatccaggaacagctgctgtattcatggttttcaagatactcttgcatcctcttataatagctcttcaagggtagaatggtggtaaagttccttggagtcaaactttcattctctggtatttcctctagaaagtccatctcgttacggagtttatccaagaagctttcaatgatgtttttattccacccggtgctggaggaattgctgcggaagatgtggaagatattctggagcatcacttcaatgacggagacagcttcatccttctctagctgtcttggatgcttcacttccataggaatcttgaagttctgtctatagctgagacagagcggtccattcatctgattcaagagctctagacattctgaaatgctgctcctttgtctgagctcaagctgcttgtagttgatggagagggctgtagggagaaaccacagcaggaatgcaggttggagaatccatctgttgaccat

SEQ ID NO: 326 - DNA, 603 bp, Sigmodon hispidus
  source 1..603: mol_type = genomic DNA
ttaggcacactggtctccactggccacacacttcaggtcccgagtcagcaggcggaagaggttgtatgtgacacaggcctccaggctgccgggggtctccttcctctgggcttcctgcagcctttgcagccagcgagagaggcggcgacttgggggcctgggctctgctgtgagctgaggctgagtacaggcctgcagctgtgattggatgtggcgcagtgtgcgaaggggctggtccaggatgggatccagggctgagtcagtcatgttctccaggaccctcagtgtcagggccagctcagccttcagggccatggggcgctcctggacctgcagctgctgcaggtcccacgccctggggaagaggcgggagctgcacctgatgtcctccttcttgagacgccactcttctatggcatccttggccttcctgaagttctgcagcgcttgcggggacagggacttgaactgggctaggtggcaatgctttgtatctggtggcagcctggtggccctgggcacagggttagcgcgggttcccgccagcaccggggccagcagcaggaggaggagcccgtggcccccagctctttctggcttcat

SEQ ID NO: 327 - DNA, 543 bp, Homo sapiens
  source 1..543: mol_type = genomic DNA
tcactgcagcagagcgctgaggcccagcagcacaatcagcagctggggcgccgcagcggagctggagtcctgggagctggggtagtacttcttgtccgcgattctcacgcttaagacctggttttctcttctcagtcgctccacctctgcagacgcgtcctgaagcttatggtttaatgtagtgatctctccctcaagctcctccactttcttttgtccttgggccttctctgcatccagggaagccattagggccatcacagtgtggttgcaggtggcggcctgggcctccacatcctgaaagcccttctgggcctcggtcagctcttgttgcaggagatgggtgacattgcgacactccatcactgcccgaaggccgtcccggcaggcctcgctgttggccttgatggtgaagataatcaagggcacccccagaatcacgatgatcaggagcaccagaattcctatccccagcagaagcttacagcgcttatccccgtcttccatgggcactctgcaatagtcatacgaagtagatgccat

SEQ ID NO: 328 - DNA, 336 bp, unidentified
  source 1..336: mol_type = genomic DNA; note = SARS-CoV-2
ttatgtgtattttaatttgactcctttgagcactggctcagagtcgtcttcatcaaatttgcagcaggatccacaagaacaacagcccttgagacaactacagcaactggtcatacagcaaagcataattgtcaccattactatggcaatcaagccagctataaaacctagccaaatgtaccatggccattttatatactgctcatactttccaagttcttggagatcgatgagagattcatttaaattcttggcaacctcattgaggcggtcaatttctttttgaatgtttacaacactagagactagtggcaataaaacaagaaaaacaaacat

SEQ ID NO: 329 - DNA, 66 bp, synthetic construct
  source 1..66: mol_type = other DNA
ggaagcggagctactaacttcagcctgctgaagcaggctggagacgtggaggagaaccctggacct

SEQ ID NO: 330 - DNA, 60 bp, synthetic construct
  source 1..60: mol_type = other DNA
gagggcagaggaagtctgctaacatgcggtgacgtcgaggagaatcctggcccaactagt

SEQ ID NO: 331 - DNA, 17 bp, synthetic construct
  source 1..17: mol_type = other DNA
taatacgactcactata

SEQ ID NO: 332 - DNA, 19 bp, synthetic construct
  source 1..19: mol_type = other DNA
taatacgactcactataat

SEQ ID NO: 333 - DNA, 19 bp, synthetic construct
  source 1..19: mol_type = other DNA
taatacgactcactataag

SEQ ID NO: 334 - DNA, 19 bp, synthetic construct
  source 1..19: mol_type = other DNA
taatacgactcactatagg

SEQ ID NO: 335-336 - skipped (2 entries)

SEQ ID NO: 337 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 338 - DNA, 105 bp, unidentified
  source 1..105: mol_type = genomic DNA; note = Hepatitis Delta Virus Antigenomic Ribozyme
gggtcggcatggcatctccacctcctcgcggtccgacctgggcatccgaaggaggacgtcgtccactcggatggctaagggagagctcggatcgatccgcctcga

SEQ ID NO: 339 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 340 - DNA, 39 bp, synthetic construct
  source 1..39: mol_type = other DNA
ctgatgagtccgtgaggacgaaacccggagtcccgggtc

SEQ ID NO: 341 - DNA, 51 bp, synthetic construct
  source 1..51: mol_type = other DNA
cttgtttctactctgatgagtccgtgaggacgaaacccggagtcccgggtc

SEQ ID NO: 342 - DNA, 51 bp, synthetic construct
  source 1..51: mol_type = other DNA
cttgttactactctgatgagtccgtgaggacgaaacccggagtcccgggtc

SEQ ID NO: 343 - DNA, 51 bp, synthetic construct
  source 1..51: mol_type = other DNA
taaacctttaatctgatgagtccgtgaggacgaaacccggagtcccgggtc

SEQ ID NO: 344 - DNA, 51 bp, synthetic construct
  source 1..51: mol_type = other DNA
tttttttctcgtctgatgagtccgtgaggacgaaacccggagtcccgggtc

SEQ ID NO: 345 - DNA, 51 bp, synthetic construct
  source 1..51: mol_type = other DNA
aatctaattggtctgatgagtccgtgaggacgaaacccggagtcccgggtc

SEQ ID NO: 346 - DNA, 51 bp, synthetic construct
  source 1..51: mol_type = other DNA
ttttagtgaactctgatgagtccgtgaggacgaaacccggagtcccgggtc

SEQ ID NO: 347 - DNA, 51 bp, synthetic construct
  source 1..51: mol_type = other DNA
attttttcgcgtctgatgagtccgtgaggacgaaacccggagtcccgggtc

SEQ ID NO: 348 - DNA, 51 bp, synthetic construct
  source 1..51: mol_type = other DNA
cttatttgccccctgatgagtccgtgaggacgaaacccggagtcccgggtc

SEQ ID NO: 349 - DNA, 257 bp, synthetic construct
  source 1..257: mol_type = other DNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaacc

SEQ ID NO: 350 - DNA, 188 bp, synthetic construct
  source 1..188: mol_type = other DNA
cggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 351 - DNA, 44 bp, synthetic construct
  source 1..44: mol_type = other DNA
ctcactatttgttttcgcgcccagttgcaaaaagtgtcgccacc

SEQ ID NO: 352 - DNA, 301 bp, synthetic construct
  source 1..301: mol_type = other DNA
aactgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 353 - DNA, 252 bp, Homo sapiens
  source 1..252: mol_type = genomic DNA
acgggccggcccctgcgtgtggcacgggcggccgggagggcgtccccggcccggcgctgctcccgcgtgtgtcctggggttgaccagagggccccgggcgctccgtgtgtggctgcgatggtggcgtttttggggacaggtgtccgtgtcgcgcgtcgcctgggccggcggcgtggtcggtgacgcgacctcccggccccgggggaggtatatctttcgctccgagtcggcattttgggccgccgggttatt

SEQ ID NO: 354 - DNA, 330 bp, Mus musculus
  source 1..330: mol_type = genomic DNA
ctggagctttggatctttttttttttttttttttcttttttcctccagaagccctctctgtccctgtcaccgggggccctgtacgtctgaggccgagggaaagctatgggcgcggttttctttcattgacctgtcggtcttatcagttctcccggttgtcaggtcgaccagttgttccttgaggtccggttcttttcgttatggggtcatttttgggccacctccacaggtatgacttcccaggtattctctgtggcctgtcactttcctccctgtctcttttatgcttgtgatcttttctatctgttcctattggacctggagataggt

SEQ ID NO: 355 - DNA, 34 bp, Mus musculus
  source 1..34: mol_type = genomic DNA
cccccccaacttcggaggtcgaccagtactccgg

SEQ ID NO: 356 - DNA, 29 bp, Mus musculus
  source 1..29: mol_type = genomic DNA
ccaacttcggaggtcgaccagtactccgg

SEQ ID NO: 357 - DNA, 30 bp, Mus musculus
  source 1..30: mol_type = genomic DNA
cccaacttcggaggtcgaccagtactccgg

SEQ ID NO: 358 - DNA, 2200 bp, synthetic construct
  source 1..2200: mol_type = other DNA
ggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggtaccatgcaggtcaattctag

SEQ ID NO: 359 - DNA, 3435 bp, synthetic construct
  source 1..3435: mol_type = other DNA
ggccgcactagggacaggattggtgacagaaaagccccatccttaggcctaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcctcgagactagttctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 360 - DNA, 4861 bp, synthetic construct
  source 1..4861: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcctcgagactagttctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacc

SEQ ID NO: 361 - DNA, 705 bp, synthetic construct
  source 1..705: mol_type = other DNA
ggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 362 - DNA, 4861 bp, synthetic construct; identical to SEQ ID NO: 360
  source 1..4861: mol_type = other DNA

SEQ ID NO: 363 - DNA, 1053 bp, synthetic construct
  source 1..1053: mol_type = other DNA
tcgcggccaagggcgaattctgcagatatccatcacactggcggccgcaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 364 - DNA, 5540 bp, synthetic construct
  source 1..5540: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcctcgagactagttctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccagtgctggtgggtccgccggcggggagggcagaggaagtctgctaacatgcggtgacgtcgaggagaatcctggcccaactagtgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacc

SEQ ID NO: 365 - DNA, 1053 bp, synthetic construct; identical to SEQ ID NO: 363
  source 1..1053: mol_type = other DNA

SEQ ID NO: 366 - DNA, 2618 bp, synthetic construct
  source 1..2618: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccgccggcaccggttcgaggcggatcgatccgagctctcccttagccatccgagtggacgacgtcctccttcggatgcccaggtcggaccgcgaggaggtggagatgccatgccgaccc

SEQ ID NO: 367 - DNA, 3881 bp, synthetic construct
  source 1..3881: mol_type = other DNA
acggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgctcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcactcactatttgttttcgcgcccagttgcaaaaagtgtcgccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccagtgctggtgggtccgccggcggggagggcagaggaagtctgctaacatgcggtgacgtcgaggagaatcctggcccaactagtgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 368 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 369 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 370 - protein, 196 aa, Homo sapiens
MTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 371 - protein, 196 aa, Homo sapiens; identical to SEQ ID NO: 370

SEQ ID NO: 372 - protein, 200 aa, Homo sapiens
MKLDMTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 373 - protein, 200 aa, Homo sapiens
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 374 - protein, 182 aa, Homo sapiens
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 375 - protein, 193 aa, Mus musculus
MLLLLLPLLLAAVLTRTQADPVPRATRLPVEAKDCHIAQFKSLSPKELQAFKKAKDAIEKRLLEKDLRCSSHLFPRAWDLKQLQVQERPKALQAEVALTLKVWENMTDSALATILGQPLHTLSHIHSQLQTCTQLQATAEPRSPSRRLSRWLHRLQEAQSKETPGCLEASVTSNLFRLLTRDLKCVANGDQCV

SEQ ID NO: 376 - protein, 202 aa, Mus musculus
MKPETAGGHMLLLLLPLLLAAVLTRTQADPVPRATRLPVEAKDCHIAQFKSLSPKELQAFKKAKDAIEKRLLEKDLRCSSHLFPRAWDLKQLQVQERPKALQAEVALTLKVWENMTDSALATILGQPLHTLSHIHSQLQTCTQLQATAEPRSPSRRLSRWLHRLQEAQSKETPGCLEASVTSNLFRLLTRDLKCVANGDQCV

SEQ ID NO: 377 - protein, 193 aa, Mus musculus
MLLLLLPLLLAAVLTRTQADPVPRATRLPVEAKDCHIAQFKSLSPKELQAFKKAKGAIEKRLLEKDMRCSSHLISRAWDLKQLQVQERPKALQAEVALTLKVWENINDSALTTILGQPLHTLSHIHSQLQTCTQLQATAEPKPPSRRLSRWLHRLQEAQSKETPGCLEDSVTSNLFQLLLRDLKCVASGDQCV

SEQ ID NO: 378 - protein, 248 aa, Mus musculus
MLLLLLPLLLAAVLTRTQADPVPRATRLPVEAKDCHIAQFKSLSPKELQAFKKAKGAIEKRLLEKDMRCSSHLISRAWDLKQLQVQERPKALQAEVALTLKVWENINDSALTTILGQPLHTLSHIHSQLQTCVSPGSLAPPQCSPATWASPVSQQQPHSPHPLSTDTASGHSRAQAPESPPLPLAAQAPGGPEQGDSWLPGGLCHLQPVSTAPPGPQVCGQWRPVCLTWNPPASFRNSSLCGCTKTTP

SEQ ID NO: 379 - protein, 180 aa, Mesocricetus auratus
MTNRWILQAAFLLWFLPISLSINYEELEARQSRSNSECEDLLKQLRGPLCLRDRKFFKMPMEVNHPEQMEKRDTAFIIQEMLQNIFLVFNNTFSSTGWNKTIVESFLGKLHNQIVFLNETLKEVPEDESLTTRISLKSYYWQLGRYLKEKRYSSCAWMVTRAAVSRNFLIIRRLTWIFQS

SEQ ID NO: 380 - protein, 180 aa, Mesocricetus auratus
MTNRWILQAAFLLWFLPISLSINYEELEARQSRSNSECEDLLKQLRGPLCLRDRKFFKMPMEVNHPEQMEKRDAAFIIQEMLQNIFLVFNNTFSSTGWNKTIVESFLGKLHNQIVFLNETLKEVPEDESLTTRISLKSYYWQLGRYLKEKRYSSCAWMVTRAAVSRNFLIIRRLTWIFQS

SEQ ID NO: 381 - protein, 212 aa, Mesocricetus auratus
MPSSSQTPESSMKPAGGRLLLLLLLLLLLAAVLTGARAHPVPRATRLPPDAKDCHIARFKSLPPQELQAFKKAKDAIEEWLLKKDVRCSSRLFPRAWDLKQLQVQERPEALQAELTLTVKVLGNVTDPGLGPILDQPLLTLSHIHSQLQACTQPQPAAEPRPPSRRLSRWLHRLQEAQEKETPGCLEVSVTSNLFRLLIRDLKCVASGDQCD

SEQ ID NO: 382 - protein, 226 aa, Mesocricetus auratus
MCKPGLKSREPCSSGDTWDLTMATLFATGSGDHDAGLGHSLSCPSFQAPSRLRRGCHISIFQSISQPELDVFKNAPEALEESLPQKKVQCGTRLSPGGWKLAQLPVQERPEALQAELNLTLKVLENVTDPGLGPILDQPLLTLNHIHSQLQACTQPQPAAEPRPPSRRLSRWLHRLQEAQEKETPGCLEVSVTSNLFRLLIRDLKCVASGNQCDSVSWPGTAYPRL

SEQ ID NO: 383 - protein, 212 aa, Mesocricetus auratus
MPSSSQTPESSMKPAGGRLLLLLLLLLLLAAVLTGARAHPVPRATRLPPDAKDCHIARFKSLPPQELQAFKKAKDAIEEWLLKKDVRCSSRLFPRAWDLKQLQVQERPEALQAELTLTLKVLENVTDPGLGPILDQPLLTLSHIHSQLQACTQPQPAAEPRPPSRRLSRWLHRLQEAQEKETPGCLEVSVTSNLFRLLIRDLKCVASGDQCD

SEQ ID NO: 384 - protein, 191 aa, Mustela putorius
MTMAMVWVLLLVTAGLSMATAGPVPTSKPTTTWKGCDIGRFKSLSPRELEAFKMAKDALGNSLKNWSCSSRLFPRNRDLRQLQVWERPVALEAELTLTLKVLETMADRSQGSILDQPLHTLRHIHSELQACVKAQPTVGPHPQGRLHHWLHRLHEASKKESQGCLEASVLFNLFRLLKKDLECVASGDLCV

SEQ ID NO: 385 - protein, 190 aa, Mustela putorius
MATAWVLVLVTLLLGLTGAGSVPTSNPTSTRRGCLMSRFQSLSPSELKAIRKAKDALENALSLKNWSCGSCLFPQDRDLRVLQAWERPVALEAELALTLKVLGAMADSSLGVVLDQPLRLLRHVHSELQACVPALPTAGPRARGLLHHWLHRLQKAPKESQGCLEASVTFNLLRLLTWDLKCVASGDMCV

SEQ ID NO: 386 - protein, 192 aa, Mustela putorius
MAVGCLPVLIAMVLTRTEAVPGPKPLKVFPDAGGCHLAQFQSLSPQELQAFKKAKDTFEESLSLKAWSCRPRLFPRTWDLQQLQVGERPVALEAEVALTLKVLETMADRSLGSILDQPLHTLRHIQSELQACVEAQPLAGPRPRGRLHHWLHRLHEAPKKEPLGCLENSVMFNLFRLLTRDLKCVASGDLCA

SEQ ID NO: 387 - protein, 186 aa, Mustela putorius
MTSRCILQTALLVYFSTTVLAMNYNLLRFQLSSSSVECQELLLQLNGTSKDCLKDRMNFKIPEEIQKSQKFQKEDIVLVTLEMFQKTSDIFRRNLSSTGWNESIVENLLATLHWQKEHLEEILEDIMQEENVTWDHRTLLHLKRYYLRIVRYLKAKEFSVCAWTIVQAEILRSFFFLDKLTDSLPN

SEQ ID NO: 388 - protein, 190 aa, Sigmodon hispidus
MSRSCAFLTFLLVMSYWTTCSLGCDLPQIHNLRNNTAFTLLVQMRTLFPLSCLKDRKDFASPLEKVVAQQLQKAQAILVSSEVIQQILNIFTSNESSAAWETTLLETFCDSLYQKLRDLQDCLMQQEEVKEPTLSEEDSLGTVRQYFHRITVYLKERKHSPCAWEVVRAEVWRALSSSVKLLARWSEEKK

SEQ ID NO: 389 - protein, 181 aa, Sigmodon hispidus
MVNRWILQPAFLLWFLPTALSINYKQLELRQRSSISECLELLNQMNGPLCLSYRQNFKIPMEVKHPRQLEKDEAVSVIEVMLQNIFHIFRSNSSSTGWNKNIIESFLDKLRNEMDFLEEIPENESLTPRNFTTILPLKSYYKRMQEYLENHEYSSCSWIVVRTEIRRNFSIMGRLIKIFQN

SEQ ID NO: 390 - protein, 200 aa, Sigmodon hispidus
MKPERAGGHGLLLLLLAPVLAGTRANPVPRATRLPPDTKHCHLAQFKSLSPQALQNFRKAKDAIEEWRLKKEDIRCSSRLFPRAWDLQQLQVQERPMALKAELALTLRVLENMTDSALDPILDQPLRTLRHIQSQLQACTQPQLTAEPRPPSRRLSRWLQRLQEAQRKETPGSLEACVTYNLFRLLTRDLKCVASGDQCA

SEQ ID NO: 391 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 392 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 393 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 394 - protein, 290 aa, Homo sapiens
MIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHDEVDGGSPMENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETD

SEQ ID NO: 395 - protein, 153 aa, Homo sapiens; identical to SEQ ID NO: 391

SEQ ID NO: 396 - protein, 111 aa, unidentified
  source 1..111: note = SARS-CoV-1
MFVFLVLLPLVSSVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLKYT

SEQ ID NO: 397 - protein, 240 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPG

SEQ ID NO: 398 - protein, 329 aa, Homo sapiens
PMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 399 - protein, 349 aa, Homo sapiens
MEAHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSPCTA

SEQ ID NO: 400 - protein, 391 aa, unidentified
  source 1..391: note = Respiratory Syncytial Virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 401 - protein, 126 aa, unidentified
  source 1..126: note = Respiratory Syncytial Virus
MEKFAPEFHGEDANNRATKFLESIKGKFTSPKDPKKKDSIISVNSIDIEVTKESPITSNSTIINPTNETDDTAGNKPNYQRKPLVSFKEDPTPSDNPFSKLYKETIETFDNNEEESSYSYEEINDQ

SEQ ID NO: 402 - protein, 422 aa, unidentified
  source 1..422: note = SARS-CoV-1
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 403 - protein, 187 aa, unidentified
  source 1..187: note = SARS-CoV-1
MAPIKGVTFGEDTVWEVQGYKNVRITFELDERVDKVLNEKCSVYTVESGTEVTEFACVVAEAVVKTLQPVSDLLTNMGIDLDEWSVATFYLFDDAGEENFSSRMYCSFYPPDEEEEDDAECEEEEIDETCEHEYGTEDDYQGLPLEFGASAETVRVEEEEEEDWLDDTTEQSEIEPEPEPTPEEPVN

SEQ ID NO: 404 - protein, 419 aa, unidentified
  source 1..419: note = SARS-CoV-1
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 405 - protein, 207 aa, unidentified
  source 1..207: note = SARS-CoV-1
MAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIE

SEQ ID NO: 406 - protein, 19 aa, synthetic construct
ATNFSLLKQAGDVEENPGP

SEQ ID NO: 407 - DNA, 22 bp, synthetic construct
  source 1..22: mol_type = other DNA
tcctgctcaacttcctgtcgag

SEQ ID NO: 408 - DNA, 22 bp, synthetic construct
  source 1..22: mol_type = other DNA
caggtcaaacctcctaggaatg

SEQ ID NO: 409 - DNA, 20 bp, synthetic construct
  source 1..20: mol_type = other DNA
atgtcaataccccggctgac

SEQ ID NO: 410 - DNA, 20 bp, synthetic construct
  source 1..20: mol_type = other DNA
ggctctactacgcgatcctg

SEQ ID NO: 411 - DNA, 55 bp, synthetic construct
  source 1..55: mol_type = other DNA
gcaacatatgtaatacgactcactatagggactcactatttgttttcgcgcccag

SEQ ID NO: 412 - DNA, 143 bp, synthetic construct
  source 1..143: mol_type = other DNA
ttttttttttttttttttttttttttttttttttttttttttttttttttttttttttttttttttttttttttttttttttttttttttttttttttttttttttttttttttttttttctctttgccgtatcccatcttag

SEQ ID NO: 413 - DNA, 635 bp, synthetic construct
  source 1..635: mol_type = other DNA
agtagaaacaagggtgtttttcctcatatctctgaaattctaatcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccaagggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccaactctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccattttggttgtttttattttcccctgtttctact

SEQ ID NO: 414 - DNA, 888 bp, synthetic construct
  source 1..888: mol_type = other DNA
aggacgagaaaaaaagtgtcaaaaactaatatctcgtaatttagttaatacacatataaaccaattagattagggtttaaatttattcctccaagattaaaatgataactttaggattagttcactaaaagttatttaaaaaatcgtacgatttttaatttttaataactataattgaatacagtgttagtgtatagctatgggaatttttattataagatctttattcattagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccaagggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccaactctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatcgctaagcaagggagttaaatttaagtggtacttatcaaattcttatttgccccatttttttggttacgagatattagtttttgacactttttttctcgt

SEQ ID NO: 415 - DNA, 32 bp, synthetic construct
  source 1..32: mol_type = other DNA
ggttgggttggctgaattgatttatttgcccc

SEQ ID NO: 416 - DNA, 2421 bp, synthetic construct
  source 1..2421: mol_type = other DNA
atattaggtttttacctacccaggaaaagccaaccaacctcgatctcttgtagatctgttctctaaccggattttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgcgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttctgacgtacttgttgttttaaataaccggataaattaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 417 - DNA, 8920 bp, synthetic construct
  source 1..8920: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccgccggcaccggttcgaggcggatcgatccgagctctcccttagccatccgagtggacgacgtcctccttcggatgcccaggtcggaccgcgaggaggtggagatgccatgccgaccctttttttttttttttttttttttttttttttttgtcattctcctaagaagctattaaaatcacatggggatagcactactaaaattaattttacacattagggctcttccatataggcagctctccctagcattattcactgtaccctcgatcgtactccgcgtggcctcgatgaaaatgtggtggctctttcaagtcctccctaatgttacacattgattaaagattgctatgtgagattaaagttaactaaacctacttgtgctgtttagttacgagaattcattctgcacaagagtagactatgtatcgtaaacggaattgcgaaaacgtttacatagcccatctgccttgtgtggtcatcatgagtttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccaagggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccaactctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccattttaatttatccggttatttaaaacaacaagtacgtcagaaatctccataaacgatgtgttcgaaggcatagccctcgagcttatatcgctgtatgaattcatccatagcgagctcgagaaagtcagtttccatttgtgatctgggcttaaaatcctctaagtctctgctctgagtaaagtaggtttcaggcaactgttgaataatgccgtctactttcttaaagtagttaaactgtgtttttactgattctccaattaatgtgactccattgacgctagcttgtgctggtccctttgaaggtgttagacctttgactgaaccttctgttattaaaacaccattacgggcgtttctaaaaaggtctacctgtccttccactctaccatcaaacaagacagtaagtgaagaacaagcactctcagtaggtttcttggcaatgtcagtcattgtgcagacacctattgtagatacatgtgctggggcttctcttttgtagtcccagattacagtattagcagcgatatcaacacccaaattattgagtatcttaatctctggcactggtttaatgttacgcttagcccaaagctcaaatgcaacattaacaggaagtgttgtcttattttcaaagatctccacatcaataccatctacctttgtgtaaacagcattattaatgatggaaacaggtgcttcgccggcgcgagtgagttcacggagtgcaccactgccatgcttagtgttccagttttgttcataatcttcaatgggatcagtgccaagctcgtcacctaagtcataagactttagatcgatgccatagctatgaccaccggctcccttattaccgttcttacgaagaagaacattgcggtatgcaattggggtttcgcccacatgtggcacgagtactcccagtgttataccgctacgaccgtactgaatgccgtccatttctgcaaccagctcaacgaccttgtggccgtgattggtgcttaaggcatcagaacgtttaatgaacacatagggctgttcaagctggggcagtacgcctttttccagctctactagaccacaagtgccatttttgaggtgttcacgtgcctccgatagggcctcttccacagagtccccgaagccacgcactagcacgtctctaacctgaaggacaggcaaactgagttggacgtgtgttttctcgttgacaccaagaacaaggctctccatcttacctttcggtcacacccggacgaaacctaggtatgctgatgatcgactgcaacacggacgaaaccgtaagcagtctgcagaagagggacgagttactcgtttcttgtcaacgacagtaaaatttattattgtttatactgcgtaggtgcactaggcatgcagccgagcgacagctacacagattttaaaatccggttagagaacagatctacaagagatcgaggttggttggcttttcctgggtaggtaaaaacctaatatacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgctcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcactcactatttgttttcgcgcccagttgcaaaaagtgtcgccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccagtgctggtgggtccgccggcggggagggcagaggaagtctgctaacatgcggtgacgtcgaggagaatcctggcccaactagtgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 418 - DNA, 8926 bp, synthetic construct
  source 1..8926: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccgccggcaccggttcgaggcggatcgatccgagctctcccttagccatccgagtggacgacgtcctccttcggatgcccaggtcggaccgcgaggaggtggagatgccatgccgaccctttttttttttttttttttttttttttttttttgtcattctcctaagaagctattaaaatcacatggggatagcactactaaaattaattttacacattagggctcttccatataggcagctctccctagcattattcactgtaccctcgatcgtactccgcgtggcctcgatgaaaatgtggtggctctttcaagtcctccctaatgttacacattgattaaagattgctatgtgagattaaagttaactaaacctacttgtgctgtttagttacgagaattcattctgcacaagagtagactatgtatcgtaaacggaattgcgaaaacgtttacatagcccatctgccttgtgtggtcatcatgagttcagtttcggaggtaacctgtaagtctgttaatgaagtaaaagttccttaggatttccactctgactatggtccaggcacagtgactgtactccttggccttcaggtaatgcagaatcctcccataatatcttttcaggtgcagactgctcatgagttttcccctggtgaaatcttctttctccagtttttcttccaggactgtcttcagatggtttatctgatgatagacattagccaggaggttctcaacaatagtctcattccagccagtgctagatgaatcttgtctgaaaatagcaaagatgttctggagcatctcatagatggtcaatgcggcgtcctccttctggaactgctgcagctgcttaatctcctcagggatgtcaaagttcatcctgtccttgaggcagtattcaagcctcccattcaattgccacaggagcttctgacactgaaaattgctgcttctttgtaggaatccaagcaagttgtagctcatggaaagagctgtagtggagaagcacaacaggagagcaatttggaggagacacttgttggtcattttaatttatccggttatttaaaacaacaagtacgtcagaaatctccataaacgatgtgttcgaaggcatagccctcgagcttatatcgctgtatgaattcatccatagcgagctcgagaaagtcagtttccatttgtgatctgggcttaaaatcctctaagtctctgctctgagtaaagtaggtttcaggcaactgttgaataatgccgtctactttcttaaagtagttaaactgtgtttttactgattctccaattaatgtgactccattgacgctagcttgtgctggtccctttgaaggtgttagacctttgactgaaccttctgttattaaaacaccattacgggcgtttctaaaaaggtctacctgtccttccactctaccatcaaacaagacagtaagtgaagaacaagcactctcagtaggtttcttggcaatgtcagtcattgtgcagacacctattgtagatacatgtgctggggcttctcttttgtagtcccagattacagtattagcagcgatatcaacacccaaattattgagtatcttaatctctggcactggtttaatgttacgcttagcccaaagctcaaatgcaacattaacaggaagtgttgtcttattttcaaagatctccacatcaataccatctacctttgtgtaaacagcattattaatgatggaaacaggtgcttcgccggcgcgagtgagttcacggagtgcaccactgccatgcttagtgttccagttttgttcataatcttcaatgggatcagtgccaagctcgtcacctaagtcataagactttagatcgatgccatagctatgaccaccggctcccttattaccgttcttacgaagaagaacattgcggtatgcaattggggtttcgcccacatgtggcacgagtactcccagtgttataccgctacgaccgtactgaatgccgtccatttctgcaaccagctcaacgaccttgtggccgtgattggtgcttaaggcatcagaacgtttaatgaacacatagggctgttcaagctggggcagtacgcctttttccagctctactagaccacaagtgccatttttgaggtgttcacgtgcctccgatagggcctcttccacagagtccccgaagccacgcactagcacgtctctaacctgaaggacaggcaaactgagttggacgtgtgttttctcgttgacaccaagaacaaggctctccatcttacctttcggtcacacccggacgaaacctaggtatgctgatgatcgactgcaacacggacgaaaccgtaagcagtctgcagaagagggacgagttactcgtttcttgtcaacgacagtaaaatttattattgtttatactgcgtaggtgcactaggcatgcagccgagcgacagctacacagattttaaaatccggttagagaacagatctacaagagatcgaggttggttggcttttcctgggtaggtaaaaacctaatatacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgctcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcactcactatttgttttcgcgcccagttgcaaaaagtgtcgccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccagtgctggtgggtccgccggcggggagggcagaggaagtctgctaacatgcggtgacgtcgaggagaatcctggcccaactagtgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 419 - DNA, 55 bp, synthetic construct
  source 1..55: mol_type = other DNA
aggacgagaaaaaaagtgtcaaaaactaatatctcgtaatttatttttaataact

SEQ ID NO: 420 - DNA, 55 bp, synthetic construct
  source 1..55: mol_type = other DNA
atttgccccatttttttggttacgagatattagtttttgacactttttttctcgt

(19) United States
(12) Patent Application Publication
(10) Pub. No.: US 2026/0250710 A1
(43) Pub. Date: Aug. 27, 2026

(54) Encrypted RNA and Methods of Its Use

(71) Applicant: Autonomous Therapeutics, Inc., Rockville, MD (US)

(72) Inventors: Timothy Notton, Rockville, MD (US); Andrii Kaberniuk, Rockville, MD (US); Anton Shemetov, Rockville, MD (US); Sayantan Bose, Rockville, MD (US); Mikhail Matlashov, Rockville, MD (US); Kristina Brazhnik, Rockville, MD (US); Ariel Weinberger, Rockville, MD (US); Won-Bin Young, Rockville, MD (US); Janice Ortega Rodríguez, Rockville, MD (US)

(73) Assignee: Autonomous Therapeutics, Inc., Rockville, MD (US)

(21) Appl. No.: 18/992,412

(22) PCT Filed: Jul. 11, 2023
(86) PCT No.: PCT/US2023/069976

§ 371 Date: Jan. 8, 2025

Related U.S. Application Data
(60) Provisional application No. 63/388,110, filed on Jul. 11, 2022.
(60) Provisional application No. 63/390,245, filed on Jul. 18, 2022.
(60) Provisional application No. 63/390,139, filed on Jul. 18, 2022.
(60) Provisional application No. 63/493,906, filed on Apr. 3, 2023.

(51) Int. Cl. C12N 15/86; A61K 38/00; C07K 14/555; C07K 14/565; C12N 15/82; C12P 19/34

(52) U.S. Cl. CPC C12N 15/86; C07K 14/555; C07K 14/565; C12N 15/8283; C12P 19/34; A61K 38/00; C12N 2740/15043

(57) ABSTRACT

Provided herein are encrypted RNAs, and DNAs that encode encrypted RNAs, that enable increased translation of polypeptides, including therapeutic polypeptides, after being contacted by translation activators, and methods of their use.

### CROSS-REFERENCE TO RELATED APPLICATIONS

This application claims the benefit under 35 U.S.C. § 119 (e) of U.S. Provisional Application No. 63/388,110, filed Jul. 11, 2022, entitled “ENCRYPTED RNA AND METHODS OF ITS USE,” U.S. Provisional Application No. 63/390,139, filed Jul. 18, 2022, entitled “ENCRYPTED RNA AND METHODS OF ITS USE,” U.S. Provisional Application No. 63/390,245, filed Jul. 18, 2022, entitled “ENCRYPTED RNA AND METHODS OF ITS USE,” and U.S. Provisional Application No. 63/493,906, filed Apr. 3, 2023, entitled “ENCRYPTED RNA AND METHODS OF ITS USE,” the entire disclosure of each of which is hereby incorporated by reference in its entirety.

### REFERENCE TO AN ELECTRONIC SEQUENCE LISTING

The contents of the electronic sequence listing (A137870001WO00-SEQ-CEW.xml; Size: 647,938 bytes; and Date of Creation: Jul. 11, 2023) is herein incorporated by reference in its entirety.

### FIELD OF THE INVENTION

The disclosure relates to encrypted RNAs, and DNAs that encode encrypted RNAs, that enable increased translation of polypeptides, including therapeutic polypeptides, after being contacted by translation activators, and methods of their use.

### BACKGROUND

Currently, the two major structural classes of FDA-approved drugs are small molecules and proteins. Small-molecule drugs, which consist predominantly of hydrophobic organic compounds, typically act by deactivating or inhibiting target proteins through competitive binding. However, the proteins that might possess such binding pockets have been estimated to account for only 2-5% of the protein-coding human genome (Hopkins A L. et al. Nat Rev Drug Discov. 2002; 1:727-30). Protein-based drugs (e.g., antibodies), by contrast, can bind with high specificity to a variety of targets or be used to replace mutated or missing proteins (e.g., delivering insulin for diabetes). However, the size, specificity, and stability of proteins limit their utility towards many potential disease targets.

The mRNA and DNA precursors of proteins, however, are promising therapeutically in that they can be specifically targeted via Watson-Crick base pairing and, in the case of gene editing, which aims to permanently change the host's DNA, represent an avenue to cure a genetic defect as opposed to just treating it. Over the past few decades, RNA drugs have emerged as candidates to address diseases at the gene and RNA levels. Although it has been known since 1990 that nucleic acids can be used to modulate protein production in vivo (Wolff J A, et al. Science. 1990; 247:1465-8), therapeutic RNA delivery has been limited by a number of factors. Naked, single-stranded RNA is prone to nuclease degradation, can activate the immune system, and is too large and negatively charged to passively cross the cell membrane—and can require additional means of cellular entry and escape from endosomes, which transport extracellular nanoparticles into the cytoplasm (Sahay G, et al. J Control Release. 2010; 145:182-95). As such, the nucleic acid delivery field has centered on the design of delivery methods and materials that will transport RNA drugs to the site of interest.

Despite the recent successes of RNA therapeutics and vaccines, there is still a need in the art to identify RNA medicines that are safe and effective in treating disease, including RNA medicines that have increased disease- or target-specificity.

### SUMMARY

Provided herein are isolated RNA polynucleotides comprising a coding region having a coding and template regions, wherein the template regions comprise two distinct regions, a left flanking region (“L region”) of a virus and a right flanking region (“R region”) of the virus. The present disclosure is related to “encrypted RNA” that encodes a polypeptide of interest, which is translated at reduced levels until the encrypted RNA is contacted by a “target-specific translation activator”. The target-specific translation activator directs increased translation of the polypeptide of interest by transcribing the encrypted RNA into a distinct mRNA species that is more translatable by the cellular ribosomal machinery. In some embodiments, an encrypted RNA encodes a therapeutic polypeptide of interest. The present disclosure is also related to DNA that encodes encrypted RNA. In some embodiments, the target-specific translation activator comprises an RNA-dependent RNA polymerase or an RNA-dependent DNA polymerase.

Aspects of the present disclosure provide isolated RNA polynucleotides, comprising a coding region having a coding sequence encoding one or more therapeutic polypeptides; and template regions, wherein the template regions comprise two distinct regions, a left flanking region (“L region”) of a virus and a right flanking region (“R region”) of the virus, wherein the L region is adjacent to and contiguous with a 5′ end of the coding region and the R region is adjacent to and contiguous with a 3′ end of the coding region; wherein the coding sequence is in an antisense orientation; wherein the therapeutic polypeptide is heterologous to the virus; and wherein the template regions interact with and initiate RNA-dependent polymerase activity of a polymerase in a cell containing the RNA dependent polymerase.

In some embodiments, the present disclosure provides the reverse complement of the isolated RNA polynucleotides described herein.

In some embodiments, the virus is selected from the group consisting of viruses in the orders of Amarillovirales, Articulavirales, Blubervirales, Bunyavirales, Hepelivirales, Martellivirales, Mononegavirales, Nidovirales, and Picornavirales. In some embodiments, the virus is selected from the group consisting of viruses in the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, Rhabdoviridae, and Togaviridae. In some embodiments, the virus is selected from the group consisting of Alphacoronavirus 229E, Alphacoronavirus NL63, Alphacoronavirus WA2028, Avian metapneumovirus (AMPV), Betacoronavirus HKU1, Betacoronavirus HKU15, Betacoronavirus HKU33, Betacoronavirus OC43, Chikungunya virus, Crimean-Congo Hemorrhagic Fever Virus, Dengue Virus, Eastern Equine Encephalitis Virus (EEEV), Enterovirus D68 (EV-D68), Foot and Mouth Disease Virus, Hanta Virus, Hendra Virus, Hepatitis B Virus, Hepatitis C Virus, HMPV, Human Parainfluenzavirus 1 (HPIV1), Human Parainfluenzavirus 3 (HPIV3), Infectious Salmon Anemia Virus, Influenza A Virus, Influenza B Virus, Lassa Virus, Marburg Virus, Middle East Respiratory Syndrome Coronavirus (MERS-CoV), Newcastle Disease Virus (NDV), Nipah Virus, Norwalk Virus, Rabies Virus, Respiratory Syncytial Virus, Reston Ebola virus, Rhinovirus, Rift Valley Fever Virus, Rubella virus, SARS-COV-1, SARS-COV-2, Sudan Ebola virus, Venezuelan Equine Encephalitis Virus (VEEV), Vesicular Stomatitis Virus, Western Equine Encephalitis Virus (WEEV), Yellow Fever Virus, Zaire Ebola virus, and Zika Virus.

In some embodiments, the virus is not an alphavirus. In some embodiments, the template regions are native to the virus. In some embodiments, the template regions are variants of template regions native to the virus, wherein the variants have at least 90%, 91%, 92%, 93%, 94%, 95%, 96%, 97%, 98%, or 99% sequence identity to the template regions native to the virus. In some embodiments, each of the L and the R regions of the template regions comprise fewer than 10, 9, 8, 7, 6, 5, 4, 3, or 2 variations relative to template regions native to the virus. In some embodiments, each of the L and the R regions of the template regions vary from template regions native to the virus by not more than 10, 9, 8, 7, 6, 5, 4, 3, or 2 substitutions that are not involved in 5′ capping. In some embodiments, each of the L and the R regions of the template regions vary from template regions native to the virus by not more than 1 substitution that is not involved in 5′ capping.

In some embodiments, the isolated RNA polynucleotide comprises at least one nucleoside modification. In some embodiments, the level of nucleoside modification can refer to the level of modification across the full isolated polynucleotide, or a portion thereof (e.g., the template regions). In some embodiments, the template regions are nucleoside modified, wherein the percentage of modified nucleosides is not more than 40%, 35%, 30%, 25%, 20%, 15%, 10%, or 5%. In some embodiments, the template regions are nucleoside modified, wherein the percentage of modified nucleosides at least 30%, 35%, 40%, 45%, 50%, 55%, 60%, 65%, 70%, 75%, 80%, 90%, 95%, or 100%.

In some embodiments, the nucleoside modification is a nonimmunogenic uridine modification, and the percentage of modified uridine modifications is not more than 40%, 35%, 30%, 25%, 20% 15% or 10%. In some embodiments, the nucleoside modification is a nonimmunogenic uridine modification, and the percentage of modified uridine modifications is more than 30%, 35%, 40%, 45%, 50%, 55%, 60%, 65%, 70%, 75%, 80%, 90%, or 95%, or is 100%.

In some embodiments, the nucleoside modification is a nonimmunogenic cytidine modification, and the percentage of modified cytidine modifications is not more than 40%, 35%, 30%, 25%, 20% 15% or 10%. In some embodiments, the nucleoside modification is a nonimmunogenic cytidine modification, and the percentage of modified cytidine modifications is more than 30%, 35%, 40%, 45%, 50%, 55%, 60%, 65%, 70%, 75%, 80%, 90%, or 95%, or is 100%.

In some embodiments, the nucleoside modification is a nonimmunogenic adenosine modification, and the percentage of modified adenosine modifications is between 1% and 30%. In some embodiments, the nucleoside modification is a nonimmunogenic adenosine modification, and the percentage of modified adenosine modifications is about 1%, 5%, 10%, 15%, 20%, 25%, or 30%.

In some embodiments, the isolated polynucleotide comprises a 5′ cap structure. In some embodiments, the 5′ end of the L region comprises a 5′ cap structure. In some embodiments, the 5′ end of the L region comprises one or more variations associated with a 5′ cap structure. In some embodiments, the 5′ cap structure is selected from the group consisting of Cap 0, Cap 0 (3′-O-Me), Cap 1, Cap 1 (3′-O-Me), Cap 2, Cap 2 (3′-O-Me), Anti-Reverse Cap Analog (ARCA), inosine, N1-methyl-guanosine, 2′-fluoro-guanosine, 7-deaza-guanosine, 8-oxo-guanosine, 2-amino-guanosine, locked nucleic acid guanosine (LNA-guanosine), and 2-azido-guanosine structure.

In some embodiments, the isolated polynucleotide does not comprise a 5′ cap structure (uncapped). In some embodiments, the 5′ end of the L region does not comprise a 5′ cap structure (uncapped). In some embodiments, the 5′ end of the isolated polynucleotide comprises a 5′-monophosphate, 5′-diphosphate, or 5′-triphosphate. In some embodiments, the 5′ end of the isolated polynucleotide does not comprise a 5′-phosphate (dephosphorylated).

In some embodiments, the template regions are the reverse complement of template regions native to the virus. In some embodiments, the template regions are variants of a reverse complement of a template regions native to the virus, wherein the variants have at least 90%, 91%, 92%, 93%, 94%, 95%, 96%, 97%, 98%, or 99% sequence identity to the reverse complement of the template regions native to the virus. In some embodiments, the reverse complements of each of the L and the R regions vary from the reverse complements of template regions native to the virus by not more than 10, 9, 8, 7, 6, 5, 4, 3, or 2 substitutions that are not involved in 5′ capping. In some embodiments, the reverse complements of each of the L and the R regions vary from the reverse complements of a template region native to the virus by not more than 1 substitution that is not involved in 5′ capping.

In some embodiments, the isolated RNA polynucleotide comprises at least one nucleoside modification. In some embodiments, the level of nucleoside modification can refer to the level of modification across the full isolated polynucleotide, or a portion thereof (e.g., the template regions). In some embodiments, the template regions are nucleoside-modified and the percentage of modified nucleotides is not more than 40%, 35%, 30%, 25%, 20%, 15%, 10%, or 5%. In some embodiments, the 5′ end of the reverse complement of the R region encodes a cap structure. In some embodiments, the 5′ end of the R region is capped.

In some embodiments, the therapeutic polypeptide is a secreted polypeptide. In some embodiments, the therapeutic polypeptide is selected from the group consisting of an interferon, an interferon stimulated gene, a cytokine, a chemokine, an antibody, a signaling molecule, a cytotoxic protein, a protein that causes cell death, an antineoplastic protein, an immunomodulatory protein, protein toll-like receptor agonist, or a dominant negative protein. In some embodiments, the cytokine is an inflammatory cytokine. In some embodiments, the inflammatory cytokine is TNF-α. In some embodiments, the cytokine is an anti-inflammatory cytokine. In some embodiments, the anti-inflammatory cytokine is an interleukin-1 receptor antagonist (IL-1RN). In some embodiments, the therapeutic polypeptide is an interleukin or a caspase. In some embodiments, the interleukin is IL-12A, IL-12B or IL-2. In some embodiments, the secreted protein is an antibody.

In some embodiments, the therapeutic polypeptide is an interferon. In some embodiments, the interferon is an IFN-α, IFN-β, IFN-ε, IFN-κ, IFN-ω, IFN-γ, or IFN-λ. In some embodiments, the interferon is IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-β1, IFN-ε, IFN-κ, IFN-ω1, IFN-γ, IFN-λ1 (IL28A), IFN-λ2 (IL28B), IFN-λ3 (IL29), or IFN-λ4. In some embodiments, the interferon is IFN-α, IFN-β, IFN-κ, IFN-λ1 (IL28A), IFN-λ2 (IL28B), or IFN-λ3 (IL29).

In some embodiments, the coding sequence encodes more than one therapeutic polypeptide, which may be separated by one or more ribosomal skipping sequence. In some embodiments, the coding region further comprises one or more regulatory elements selected from the group consisting of ribosomal binding site, Kozak sequence, Shine-Dalgarno sequence, ribozyme, riboswitch, promoter, microRNA binding site, and internal ribosomal entry site (IRES). In some embodiments, the one or more regulatory elements are operably linked to the coding sequence. In some embodiments, the RNA polynucleotide further comprises a polyadenylation signal and/or a 3′ poly(A) tail.

In some embodiments, the RNA-dependent polymerase is an RNA-dependent RNA polymerase. In some embodiments, the RNA-dependent polymerase is an RNA-dependent DNA polymerase. In some embodiments, the RNA-dependent polymerase is a polymerase is from the virus.

In some embodiments, the isolated RNA polynucleotide is a single stranded RNA. In some embodiments, the isolated polynucleotide is in linear form. In some embodiments, the isolated polynucleotide is in a covalently-closed circular form.

In some embodiments, the virus is an influenza virus, wherein the L region comprises a nucleotide sequence set forth as SEQ ID NO: 2; or a variant of SEQ ID NO: 2, and wherein the R region comprises the nucleotide sequence set forth as any one of SEQ ID NOs: 20, 21, 22, or 23; or a variant of any one of SEQ ID NOs: 20, 21, 22, or 23. In some embodiments, the variant of SEQ ID NO: 2 comprises a variation at one or more nucleotide positions selected from the group consisting of positions 14-26 of SEQ ID NO: 2. In some embodiments, the variant comprises a variation at one or more nucleotide positions selected from the group consisting of positions 8-15 of any one of SEQ ID NOs: 20, 21, 22, or 23. In some embodiments, the virus is an influenza virus, wherein the L region comprises a nucleotide sequence set forth as SEQ ID NO: 3; or a variant of SEQ ID NO: 3, and wherein the R region comprises the nucleotide sequence set forth as any one of SEQ ID NOs: 24, 25, 26, or 27. In some embodiments, (i) the variant of SEQ ID NO: 3 comprises a variation at one or more nucleotide positions selected from the group consisting of positions 14-35 of SEQ ID NO: 3. In some embodiments, (i) the variant of SEQ ID NO: 24 comprises a variation at one or more nucleotide positions selected from the group consisting of positions 8-12 of SEQ ID NO: 24; (ii) the variant of SEQ ID NO: 25 comprises a variation at one or more nucleotide positions selected from the group consisting of positions 8-12 of SEQ ID NO: 25; (iii) the variant of SEQ ID NO: 26 comprises a variation at one or more nucleotide positions selected from the group consisting of positions 8-12 of SEQ ID NO: 26; or (iv) the variant of SEQ ID NO: 27 comprises a variation at one or more nucleotide positions selected from the group consisting of positions 8-12 of SEQ ID NO: 27. In some embodiments, the virus is an influenza virus, wherein the L region comprises a nucleotide sequence set forth as SEQ ID NO: 4; or a variant of SEQ ID NO: 4, and wherein the R region comprises the nucleotide sequence set forth as any one of SEQ ID NOs: 28, 29, 30, or 31; or a variant of any one of SEQ ID NOs: 28, 29, 30, or 31. In some embodiments, the variant of SEQ ID NO: 4 comprises a variation at one or more nucleotide positions selected from the group consisting of positions 14-50 of SEQ ID NO: 4. In some embodiments, the variant of any one of SEQ ID NOs: 28, 29, 30, or 31 comprises a variation at one or more nucleotide positions selected from the group consisting of positions 8-12 of any one of SEQ ID NOs: 28, 29, 30, or 31.

In some embodiments, the virus is an influenza virus, wherein the L region comprises a nucleotide sequence set forth as SEQ ID NO: 1 or 5; or a variant of SEQ ID NO: 1 or 5, and wherein the R region comprises the nucleotide sequence set forth as SEQ ID NO: 18 or 19; or a variant of SEQ ID NO: 18 or 19. In some embodiments, the variant of SEQ ID NO: 1 or 5 comprises a variation at one or more nucleotide positions selected from the group consisting of positions 14-37 of SEQ ID NO: 1 or 5. In some embodiments, the variant of SEQ ID NO: 18 or 19 comprises a variation at one or more nucleotide positions selected from the group consisting of positions 8-20 of SEQ ID NO: 18 or 19. In some embodiments, the virus is an influenza virus, wherein the L region comprises a nucleotide sequence set forth as SEQ ID NO: 6; or a variant of SEQ ID NO: 6, and wherein the R region comprises the nucleotide sequence set forth as SEQ ID NO: 32 or 33; or a variant of SEQ ID NO: 32 or 33. In some embodiments, the variant of SEQ ID NO: 6 comprises a variation at one or more nucleotide positions selected from position 14 or 15 of SEQ ID NO: 6. In some embodiments, the variant of SEQ ID NO: 32 or 33 comprises a variation at one or more nucleotide positions selected from the group consisting of positions 8-33 of SEQ ID NO: 32 or 33. In some embodiments, the virus is an influenza virus, wherein the L region comprises a nucleotide sequence set forth as SEQ ID NO: 7; or a variant of SEQ ID NO: 7, and wherein the R region comprises the nucleotide sequence set forth as any one of SEQ ID NOs: 34, 35, 36, or 37; or a variant of any one of SEQ ID NOs: 34, 35, 36, or 37. In some embodiments, the variant of SEQ ID NO: 7 comprises a variation at one or more nucleotide positions selected from the group consisting of positions 14-20 of SEQ ID NO: 7. In some embodiments, the variant of any one of SEQ ID NOs: 34, 35, 36, or 37, wherein the variant comprises a variation at one or more nucleotide positions selected from the group consisting of positions 5-8 of SEQ ID NO: 34, 35, 36, or 37. In some embodiments, the virus is an influenza virus, wherein the L region comprises a nucleotide sequence set forth as SEQ ID NO: 8; or a variant of SEQ ID NO: 8 and wherein the R region comprises the nucleotide sequence set forth as any one of SEQ ID NOs: 38, 39, 40, or 41; or a variant of any one of SEQ ID NOs: 38, 39, 40, or 41. In some embodiments, the variant of SEQ ID NO: 8 comprises a variation at one or more nucleotide positions selected from position 14 or 15 of SEQ ID NO: 8. In some embodiments, the variant of any one of SEQ ID NOs: 38, 39, 40, or 41 comprises a variation at one or more nucleotide positions selected from the group consisting of positions 8-13 of SEQ ID NO: 38, 39, 40, or 41. In some embodiments, the virus is an influenza virus, wherein the L region comprises a nucleotide sequence set forth as SEQ ID NO: 9; or a variant of SEQ ID NO: 9, and wherein the R region comprises the nucleotide sequence set forth as SEQ ID NO: 42 or 43; or a variant of SEQ ID NO: 42 or 43. In some embodiments, the variant of SEQ ID NO: 9 comprises a variation at one or more nucleotide positions selected from the group consisting of positions 14-18 of SEQ ID NO: 9. In some embodiments, the variant of SEQ ID NO: 42 or 43 comprises a variation at one or more nucleotide positions selected from the group consisting of positions 8-14 of SEQ ID NO: 42 or 43. In some embodiments, the virus is an influenza virus, wherein the L region comprises a nucleotide sequence set forth as SEQ ID NO: 11; or a variant of SEQ ID NO: 11, and wherein the R region comprises the nucleotide sequence set forth as SEQ ID NO: 46 or 47; or a variant of SEQ ID NO: 46 or 47. In some embodiments, the variant of SEQ ID NO: 11 comprises a variation at one or more nucleotide positions selected from the group consisting of positions 14-81 of SEQ ID NO: 11. In some embodiments, the variant of SEQ ID NO: 46 or 47t comprises a variation at one or more nucleotide positions selected from the group consisting of positions 5-9 of SEQ ID NO: 46 or 47. In some embodiments, the virus is an influenza virus, wherein the L region comprises a nucleotide sequence set forth as SEQ ID NO: 12; or a variant of SEQ ID NO: 12, and wherein the R region comprises the nucleotide sequence set forth as any one of SEQ ID NO: 48 or 49; or a variant of any one of SEQ ID NO: 48 or 49. In some embodiments, the variant of SEQ ID NO: 12 comprises a variation at one or more nucleotide positions selected from the group consisting of positions 14-52 of SEQ ID NO: 12. In some embodiments, the variant of any one of SEQ ID NO: 48 or 49 comprises a variation at one or more nucleotide positions selected from the group consisting of positions 8-11 of SEQ ID NO: 48 or 49. In some embodiments, the virus is an influenza virus, wherein the L region comprises a nucleotide sequence set forth as SEQ ID NO: 13; or a variant of SEQ ID NO: 13 and wherein the R region comprises the nucleotide sequence set forth as SEQ ID NO: 50 or 51; or a variant of SEQ ID NO: 50 or 51. In some embodiments, the variant of SEQ ID NO: 13 comprises a variation at one or more nucleotide positions selected from the group consisting of positions 14-87 of SEQ ID NO: 13. In some embodiments, the variant of SEQ ID NO: 50 or 51 comprises a variation at one or more nucleotide positions selected from the group consisting of positions 8-17 of SEQ ID NO: 50 or 51. In some embodiments, the virus is an influenza virus, wherein the L region comprises a nucleotide sequence set forth as any one of SEQ ID NO: 10; or a variant of SEQ ID NO: 10, and wherein the R region comprises the nucleotide sequence set forth as SEQ ID NO: 44 or 45; or a variant of SEQ ID NO: 44 or 45. In some embodiments, the variant of SEQ ID NO: 10 comprises a variation at one or more nucleotide positions selected from the group consisting of positions 14-86 of SEQ ID NO: 10. In some embodiments, the variant of SEQ ID NO: 44 or 45 comprises a variation at one or more nucleotide positions selected from the group consisting of positions 8-21 of SEQ ID NO: 44 or 45. In some embodiments, the virus is an influenza virus, wherein the L region comprises a nucleotide sequence set forth as SEQ ID NO: 14; or a variant of SEQ ID NO: 14, and wherein the R region comprises the nucleotide sequence set forth as any one of SEQ ID NO: 52 or 53; or a variant of any one of SEQ ID NO: 52 or 53. In some embodiments, the variant of SEQ ID NO: 14 comprises a variation at one or more nucleotide positions selected from the group consisting of positions 14-93 of SEQ ID NO: 14. In some embodiments, the variant of any one of SEQ ID NO: 52 or 53 comprises a variation at one or more nucleotide positions selected from the group consisting of positions 8-48 of SEQ ID NO: 52 or 53. In some embodiments, the virus is an influenza virus, wherein the L region comprises a nucleotide sequence set forth as SEQ ID NO: 15; or a variant of SEQ ID NO: 15, and wherein the R region comprises the nucleotide sequence set forth as SEQ ID NO: 54 or 55; or a variant of any one of SEQ ID NO: 54 or 55. In some embodiments, the variant of SEQ ID NO: 15 comprises a variation at one or more nucleotide positions selected from the group consisting of positions 14-95 of SEQ ID NO: 15. In some embodiments, the variant of any one of SEQ ID NO: 54 or 55 comprises a variation at one or more nucleotide positions selected from the group consisting of positions 8-34 of SEQ ID NO: 54 or 55.

In some embodiments, the virus is an influenza virus, wherein the L region comprises a nucleotide sequence set forth as SEQ ID NO: 16; or a variant of SEQ ID NO: 16, and wherein the R region comprises the nucleotide sequence set forth as any one of SEQ ID NO: 56 or 57; or a variant of any one of SEQ ID NO: 56 or 57. In some embodiments, the variant of SEQ ID NO: 16 comprises a variation at one or more nucleotide positions selected from the group consisting of positions 14-81 of SEQ ID NO: 16. In some embodiments; the variant of any one of SEQ ID NO: 56 or 57 comprises a variation at one or more nucleotide positions selected from the group consisting of positions 8-12 of SEQ ID NO: 56 or 57. In some embodiments, the virus is an influenza virus, wherein the L region comprises a nucleotide sequence set forth as SEQ ID NO: 17; or a variant of SEQ ID NO: 17, and wherein the R region comprises the nucleotide sequence set forth as any one of SEQ ID NO: 58 or 59; or a variant of any one of SEQ ID NO: 58 or 59. In some embodiments, the variant of SEQ ID NO: 17 comprises a variation at one or more nucleotide positions selected from the group consisting of positions 14-22 of SEQ ID NO: 17 In some embodiments, the variant of any one of SEQ ID NO: 58 or 59 comprises a variation at one or more nucleotide positions selected from the group consisting of positions 8-32 of SEQ ID NO: 58 or 59. In some embodiments, the virus is a sarbecovirus, wherein the L region comprises a nucleotide sequence set forth as SEQ ID NO: 137; or a variant of SEQ ID NO: 137, and wherein the R region comprises the nucleotide sequence set forth as SEQ ID NO: 128; or a variant of any one of SEQ ID NO: 128. In some embodiments, the variant of SEQ ID NO: 137 comprises a variation at one or more nucleotide positions selected from the group consisting of positions 40-1557 of SEQ ID NO: 137. In some embodiments, the variant of any one of SEQ ID NO: 128 comprises a variation at one or more nucleotide positions selected from the group consisting of positions 20-30 of SEQ ID NO: 128. In some embodiments, the virus is a sarbecovirus, wherein the L region comprises a nucleotide sequence set forth as any one of SEQ ID NOs: 138, 139, 140, 141, 142, 143, or 144; or a variant of any one of SEQ ID NOs: 138, 139, 140, 141, 142, 143, or 144, and wherein the R region comprises the nucleotide sequence set forth as any one of SEQ ID NOs: 130, 136, 145, 146, or 147; or a variant of any one of SEQ ID NOs: 130, 136, 145, 146, or 147. In some embodiments, (i) the variant of SEQ ID NO: 138 comprises a variation at one or more nucleotide positions selected from the group consisting of positions 50-312 of SEQ ID NO: 138; (ii) the variant of SEQ ID NO: 139 comprises a variation at one or more nucleotide positions selected from the group consisting of positions 50-1567 of SEQ ID NO: 139; (iii) the variant of SEQ ID NO: 140 comprises a variation at one or more nucleotide positions selected from the group consisting of positions 50-1488 of SEQ ID NO: 140; (iv) the variant of SEQ ID NO: 141 comprises a variation at one or more nucleotide positions selected from the group consisting of positions 50-1593 of SEQ ID NO: 141; (v) the variant of SEQ ID NO: 142 comprises a variation at one or more nucleotide positions selected from the group consisting of positions 50-1570 of SEQ ID NO: 142; (vi) the variant of SEQ ID NO: 143 comprises a variation at one or more nucleotide positions selected from the group consisting of positions 50-1488 of SEQ ID NO: 143; or (vii) the variant of SEQ ID NO: 144 comprises a variation at one or more nucleotide positions selected from the group consisting of positions 50-1593 of SEQ ID NO: 144. In some embodiments, the variant of any one of SEQ ID NOs: 130, 136, 145, 146, or 147 comprises a variation at one or more nucleotide positions selected from the group consisting of positions 20-320 of SEQ ID NO: 130; (ii) the variant of SEQ ID NO: 136 comprises a variation at one or more nucleotide positions selected from the group consisting of positions 20-33 of SEQ ID NO: 136; (iii) the variant of SEQ ID NO: 145 comprises a variation at one or more nucleotide positions selected from the group consisting of positions 50-1461 of SEQ ID NO: 145; (iv) the variant of SEQ ID NO: 146 comprises a variation at one or more nucleotide positions selected from the group consisting of positions 60-1441 of SEQ ID NO: 146; or (v) the variant of SEQ ID NO: 147 comprises a variation at one or more nucleotide positions selected from the group consisting of positions 60-897 of SEQ ID NO: 147.

In some embodiments, the virus is Respiratory Syncytial Virus (RSV), wherein the L region comprises a nucleotide sequence set forth as any one of SEQ ID NOs: 158, 163, 165, 166, or 419; or a variant of any one of SEQ ID NOs: 158, 163, 165, 166, or 419, and wherein the R region comprises the nucleotide sequence set forth as any one of SEQ ID NOs: 169, 170, 176, 177, or 420; or a variant of any one of SEQ ID NOS: 169, 170, 176, 177, or 420. In some embodiments, (i) the variant of SEQ ID NO: 158 comprises a variation at one or more nucleotide positions selected from the group consisting of positions 15-207 of SEQ ID NO: 158; (ii) the variant of SEQ ID NO: 163 comprises a variation at one or more nucleotide positions selected from the group consisting of positions 18-210 of SEQ ID NO: 163; (iii) the variant of SEQ ID NO: 165 comprises a variation at one or more nucleotide positions selected from the group consisting of positions 15-147 of SEQ ID NO: 165; (iv) the variant of SEQ ID NO: 166 comprises a variation at one or more nucleotide positions selected from the group consisting of positions 15-32 of SEQ ID NO: 166; or (v) the variant of SEQ ID NO: 419 comprises a variation at one or more nucleotide positions selected from the group consisting of positions 18-35 of SEQ ID NO: 419. In some embodiments, (i) the variant of SEQ ID NO: 169 comprises a variation at one or more nucleotide positions selected from the group consisting of positions 15-78 of SEQ ID NO: 169; (ii) the variant of SEQ ID NO: 170 comprises a variation at one or more nucleotide positions selected from the group consisting of positions 15-80 of SEQ ID NO: 170; (iii) the variant of SEQ ID NO: 176 comprises a variation at one or more nucleotide positions selected from the group consisting of positions 15-36 of SEQ ID NO: 176; (iv) the variant of SEQ ID NO: 177 comprises a variation at one or more nucleotide positions selected from the group consisting of positions 15-33 of SEQ ID NO: 177; or (v) the variant of SEQ ID NO: 420 comprises a variation at one or more nucleotide positions selected from the group consisting of positions 15-35 of SEQ ID NO: 420.

In some embodiments, the virus is a parainfluenzavirus, wherein the L region comprises a nucleotide sequence set forth as any one of SEQ ID NOs: 181, 182, or 183; or a variant of any one of SEQ ID NOs: 181, 182, or 183, and wherein the R region comprises the nucleotide sequence set forth as SEQ ID NO: 184; or a variant of SEQ ID NO: 184. In some embodiments, (i) the variant of SEQ ID NO: 181 comprises a variation at one or more nucleotide positions selected from the group consisting of positions 17-136 of SEQ ID NO: 181; (ii) the variant of SEQ ID NO: 182 comprises a variation at one or more nucleotide positions selected from the group consisting of positions 17-142 of SEQ ID NO: 182; or (iii) the variant of SEQ ID NO: 183 comprises a variation at one or more nucleotide positions selected from the group consisting of positions 17-136 of SEQ ID NO: 183. In some embodiments, the variant of SEQ ID NO: 184 comprises a variation at one or more nucleotide positions selected from the group consisting of positions 17-98 of SEQ ID NO: 184. In some embodiments, the virus is a parainfluenzavirus, wherein the L region comprises a nucleotide sequence set forth as any one of SEQ ID NOs: 187, 188, or 189; or a variant of any one of SEQ ID NOs: 187, 188, or 189, and wherein the R region comprises the nucleotide sequence set forth as SEQ ID NO: 190; or a variant of SEQ ID NO: 190. In some embodiments, (i) the variant of SEQ ID NO: 187 comprises a variation at one or more nucleotide positions selected from the group consisting of positions 21-95 of SEQ ID NO: 187; (ii) the variant of SEQ ID NO: 188 comprises a variation at one or more nucleotide positions selected from the group consisting of positions 21-101 of SEQ ID NO: 188; or (iii) the variant of SEQ ID NO: 189 comprises a variation at one or more nucleotide positions selected from the group consisting of positions 21-95 of SEQ ID NO: 189. In some embodiments, the variant of SEQ ID NO: 190 comprises a variation at one or more nucleotide positions selected from the group consisting of positions 21-93 of SEQ ID NO: 190.

In some embodiments, the virus is a metapneumovirus, wherein the L region comprises a nucleotide sequence set forth as any one of SEQ ID NOs: 196, 197, or 199; or a variant of any one of SEQ ID NOs: 196, 197, or 199, and wherein the R region comprises the nucleotide sequence set forth as SEQ ID NO: 201; or a variant of SEQ ID NO: 201. In some embodiments, (i) the variant of SEQ ID NO: 196 comprises a variation at one or more nucleotide positions selected from the group consisting of positions 21-224 of SEQ ID NO: 196; (ii) the variant of SEQ ID NO: 197 comprises a variation at one or more nucleotide positions selected from the group consisting of positions 21-230 of SEQ ID NO: 197; or (iii) the variant of SEQ ID NO: 199 comprises a variation at one or more nucleotide positions selected from the group consisting of positions 21-140 of SEQ ID NO: 199. In some embodiments, the variant of SEQ ID NO: 201 comprises a variation at one or more nucleotide positions selected from the group consisting of positions 17-32 of SEQ ID NO: 201. In some embodiments, the virus is a metapneumovirus, wherein the L region comprises a nucleotide sequence set forth as SEQ ID NO: 195; or a variant of SEQ ID NO: 195, and wherein the R region comprises the nucleotide sequence set forth as SEQ ID NO: 200; or a variant of SEQ ID NO: 200. In some embodiments, the variant of SEQ ID NO: 195 comprises a variation at one or more nucleotide positions selected from the group consisting of positions 21-224 of SEQ ID NO: 195. In some embodiments, the variant of SEQ ID NO: 200 comprises a variation at one or more nucleotide positions selected from the group consisting of positions 17-32 of SEQ ID NO: 200.

In some embodiments, the virus is a henipavirus, wherein the L region comprises a nucleotide sequence set forth as SEQ ID NO: 204; or a variant of SEQ ID NO: 204, and wherein the R region comprises the nucleotide sequence set forth as SEQ ID NO: 206; or a variant of SEQ ID NO: 206. In some embodiments, the variant of SEQ ID NO: 204 comprises a variation at one or more nucleotide positions selected from the group consisting of positions 17-77 of SEQ ID NO: 204. In some embodiments, the variant of SEQ ID NO: 206 comprises a variation at one or more nucleotide positions selected from the group consisting of positions 17-91 of SEQ ID NO: 206. In some embodiments, the virus is a henipavirus, wherein the L region comprises a nucleotide sequence set forth as SEQ ID NO: 209 or 210; or a variant of SEQ ID NO: 209 or 210, and wherein the R region comprises the nucleotide sequence set forth as SEQ ID NO: 211; or a variant of SEQ ID NO: 211. In some embodiments, (i) the variant of SEQ ID NO: 209 comprises a variation at one or more nucleotide positions selected from the group consisting of positions 17-77 of SEQ ID NO: 209; or (ii) the variant of SEQ ID NO: 210 comprises a variation at one or more nucleotide positions selected from the group consisting of positions 17-83 of SEQ ID NO: 210. In some embodiments, the variant of SEQ ID NO: 211 comprises a variation at one or more nucleotide positions selected from the group consisting of positions 17-91 of SEQ ID NO: 211.

In some embodiments, the virus is a hepadnavirus, wherein the L region comprises a nucleotide sequence set forth as SEQ ID NO: 222 or 223; or a variant of SEQ ID NO: 222 or 223 and wherein the R region comprises the nucleotide sequence set forth as SEQ ID NO: 225; or a variant of SEQ ID NO: 225. In some embodiments, (i) the variant of SEQ ID NO: 222 comprises a variation at one or more nucleotide positions selected from the group consisting of positions 101-639 of SEQ ID NO: 222. or (ii) the variant of SEQ ID NO: 223 comprises a variation at one or more nucleotide positions selected from the group consisting of positions 101-186 of SEQ ID NO: 223. In some embodiments, the variant of SEQ ID NO: 225 comprises a variation at one or more nucleotide positions selected from the group consisting of positions 101-1023 of SEQ ID NO: 225.

In some embodiments, the virus is a filovirus, wherein the L region comprises a nucleotide sequence set forth as any one of SEQ ID NOs: 227, 228, 229, or 230; or a variant of any one of SEQ ID NOs: 227, 228, 229, or 230, and wherein the R region comprises the nucleotide sequence set forth as SEQ ID NO: 231; or a variant of SEQ ID NO: 231. In some embodiments, (i) the variant of SEQ ID NO: 227 comprises a variation at one or more nucleotide positions selected from the group consisting of positions 20-710 of SEQ ID NO: 227; (ii) the variant of SEQ ID NO: 228 comprises a variation at one or more nucleotide positions selected from the group consisting of positions 23-713 of SEQ ID NO: 228; (iii) the variant of SEQ ID NO: 229 comprises a variation at one or more nucleotide positions selected from the group consisting of positions 17-707 of SEQ ID NO: 229; or (iv) the variant of SEQ ID NO: 230 comprises a variation at one or more nucleotide positions selected from the group consisting of positions 17-707 of SEQ ID NO: 230. In some embodiments, the variant of SEQ ID NO: 231 comprises a variation at one or more nucleotide positions selected from the group consisting of positions 15-449 of SEQ ID NO: 231. In some embodiments, the virus is a filovirus, wherein the L region comprises a nucleotide sequence set forth as any one of SEQ ID NOs: 232, 233, 234, or 235; or a variant of any one of SEQ ID NOs: 232, 233, 234, or 235, and wherein the R region comprises the nucleotide sequence set forth as SEQ ID NO: 236; or a variant of SEQ ID NO: 236. In some embodiments, (i) the variant of SEQ ID NO: 232 comprises a variation at one or more nucleotide positions selected from the group consisting of positions 20-678 of SEQ ID NO: 232; (ii) the variant of SEQ ID NO: 233 comprises a variation at one or more nucleotide positions selected from the group consisting of positions 23-681 of SEQ ID NO: 233; (iii) the variant of SEQ ID NO: 234 comprises a variation at one or more nucleotide positions selected from the group consisting of positions 23-678 of SEQ ID NO: 234; or (iv) the variant of SEQ ID NO: 235 comprises a variation at one or more nucleotide positions selected from the group consisting of positions 23-678 of SEQ ID NO: 235. In some embodiments, the variant of SEQ ID NO: 236 comprises a variation at one or more nucleotide positions selected from the group consisting of positions 15-437 of SEQ ID NO: 236.

In some embodiments, the virus is a filovirus, wherein the L region comprises a nucleotide sequence set forth as any one of SEQ ID NOs: 237, 238, or 239; or a variant of any one of SEQ ID NOs: 237, 238, or 239; and wherein the R region comprises the nucleotide sequence set forth as SEQ ID NO: 240; or a variant of SEQ ID NO: 240. In some embodiments, (i) the variant of SEQ ID NO: 237 comprises a variation at one or more nucleotide positions selected from the group consisting of positions 20-605 of SEQ ID NO: 237; (ii) the variant of SEQ ID NO: 238 comprises a variation at one or more nucleotide positions selected from the group consisting of positions 21-606 of SEQ ID NO: 238; or (iii) the variant of SEQ ID NO: 239 comprises a variation at one or more nucleotide positions selected from the group consisting of positions 20-605 of SEQ ID NO: 239. In some embodiments, the variant of SEQ ID NO: 240 comprises a variation at one or more nucleotide positions selected from the group consisting of positions 15-83 of SEQ ID NO: 240. In some embodiments, the virus is a filovirus, wherein the L region comprises a nucleotide sequence set forth as SEQ ID NO: 241; or a variant of SEQ ID NO: 241, and wherein the R region comprises the nucleotide sequence set forth as SEQ ID NO: 242; or a variant of any one of SEQ ID NO: 242. In some embodiments, the variant of SEQ ID NO: 241 comprises a variation at one or more nucleotide positions selected from the group consisting of positions 20-34 of SEQ ID NO: 241. In some embodiments, the variant of SEQ ID NO: 242 comprises a variation at one or more nucleotide positions selected from the group consisting of positions 100-593 of SEQ ID NO: 242. In some embodiments, the virus is a filovirus, wherein the L region comprises a nucleotide sequence set forth as SEQ ID NO: 243; or a variant of SEQ ID NO: 243, and wherein the R region comprises the nucleotide sequence set forth as SEQ ID NO: 244; or a variant of SEQ ID NO: 244. In some embodiments, the variant of SEQ ID NO: 243 comprises a variation at one or more nucleotide positions selected from the group consisting of positions 30-45 of SEQ ID NO: 243. In some embodiments, the variant of SEQ ID NO: 244 comprises a variation at one or more nucleotide positions selected from the group consisting of positions 100-677 of SEQ ID NO: 244. In some embodiments, the virus is a filovirus, wherein the L region comprises a nucleotide sequence set forth as any one of SEQ ID NOs: 245, 246, or 247; or a variant of any one of SEQ ID NOs: 245, 246, or 247, and wherein the R region comprises the nucleotide sequence set forth as SEQ ID NO: 248; or a variant of SEQ ID NO: 248. In some embodiments, (i) the variant of SEQ ID NO: 245 comprises a variation at one or more nucleotide positions selected from the group consisting of positions 29-171 of SEQ ID NO: 245; (ii) the variant of SEQ ID NO: 246 comprises a variation at one or more nucleotide positions selected from the group consisting of positions 30-171 of SEQ ID NO: 246; or (iii) the variant of SEQ ID NO: 247 comprises a variation at one or more nucleotide positions selected from the group consisting of positions 29-171 of SEQ ID NO: 247. In some embodiments, the variant of SEQ ID NO: 248 comprises a variation at one or more nucleotide positions selected from the group consisting of positions 20-91 of SEQ ID NO: 248.

In some embodiments, the virus is an alphavirus, wherein the L region comprises a nucleotide sequence set forth as SEQ ID NO: 249; or a variant of SEQ ID NO: 249, and wherein the R region comprises the nucleotide sequence set forth as SEQ ID NO: 250 or 251; or a variant of SEQ ID NO: 250 or 251. In some embodiments, the variant of SEQ ID NO: 249 comprises a variation at one or more nucleotide positions selected from the group consisting of positions 60-274 of SEQ ID NO: 249. In some embodiments, (i) the variant of SEQ ID NO: 250 comprises a variation at one or more nucleotide positions selected from the group consisting of positions 60-183 of SEQ ID NO: 250; or (ii) the variant of SEQ ID NO: 251 comprises a variation at one or more nucleotide positions selected from the group consisting of positions 60-375 of SEQ ID NO: 251. In some embodiments, the virus is an alphavirus, wherein the L region comprises a nucleotide sequence set forth as SEQ ID NO: 255; or a variant of SEQ ID NO: 255 and wherein the R region comprises the nucleotide sequence set forth as SEQ ID NO: 256 or 257; or a variant of SEQ ID NO: 256 or 257. In some embodiments, the variant of SEQ ID NO: 255 comprises a variation at one or more nucleotide positions selected from the group consisting of positions 60-35 of SEQ ID NO: 255. In some embodiments, (i) the variant of SEQ ID NO: 256 comprises a variation at one or more nucleotide positions selected from the group consisting of positions 600-273 of SEQ ID NO: 256; or (ii) the variant of SEQ ID NO: 257 comprises a variation at one or more nucleotide positions selected from the group consisting of positions 60-377 of SEQ ID NO: 257. In some embodiments, the virus is an alphavirus, wherein the L region comprises a nucleotide sequence set forth as SEQ ID NO: 261; or a variant of SEQ ID NO: 261, and wherein the R region comprises the nucleotide sequence set forth as SEQ ID NO: 262 or 263; or a variant of SEQ ID NO: 262 or 263. In some embodiments, the variant of SEQ ID NO: 261 comprises a variation at one or more nucleotide positions selected from the group consisting of positions 60-215 of SEQ ID NO: 261. In some embodiments, (i) the variant of SEQ ID NO: 262 comprises a variation at one or more nucleotide positions selected from the group consisting of positions 60-166 of SEQ ID NO: 262; or (ii) the variant of SEQ ID NO: 263 comprises a variation at one or more nucleotide positions selected from the group consisting of positions 60-379 of SEQ ID NO: 263.

Aspects of the present disclosure provide isolated RNA polynucleotides, comprising a coding region having a coding sequence encoding one or more polypeptide; and template regions, wherein the template regions comprise two distinct regions, a left flanking region (“L region”) of a virus and a right flanking region (“R region”) of the virus, wherein the L region is adjacent to and contiguous with a 5′ end of the coding region and the R region is adjacent to and contiguous with a 3′ end of the coding region; wherein the coding sequence is in a sense orientation; wherein the template region interact with and initiate RNA-dependent polymerase activity of a polymerase in a cell containing the RNA dependent polymerase; and wherein the virus is not an alphavirus or wherein the polypeptide is heterologous to the virus.

In some embodiments, the present disclosure provides the reverse complement of the isolated RNA polynucleotides described herein.

In some embodiments, the virus is selected from the group consisting of viruses in the orders of Amarillovirales, Articulavirales, Blubervirales, Bunyavirales, Hepelivirales, Mononegavirales, Nidovirales, and Picornavirales. In some embodiments, the virus is selected from the group consisting of viruses in the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, and Rhabdoviridae. In some embodiments, the virus is from the group consisting of Alphacoronavirus 229E, Alphacoronavirus NL63, Alphacoronavirus WA2028, Avian metapneumovirus (AMPV), Betacoronavirus HKU1, Betacoronavirus HKU15, Betacoronavirus HKU33, Betacoronavirus OC43, Chikungunya virus, Crimean-Congo Hemorrhagic Fever Virus, Dengue Virus, Enterovirus D68 (EV-D68), Foot and Mouth Disease Virus, Hanta Virus, Hendra Virus, Hepatitis B Virus, Hepatitis C Virus, HMPV, Human Parainfluenzavirus 1 (HPIV1), Human Parainfluenzavirus 3 (HPIV3), Infectious Salmon Anemia Virus, Influenza A Virus, Influenza B Virus, Lassa Virus, Marburg Virus, Middle East Respiratory Syndrome Coronavirus (MERS-COV), Newcastle Disease Virus (NDV), Nipah Virus, Norwalk Virus, Rabies Virus, Respiratory Syncytial Virus, Reston Ebola virus, Rhinovirus, Rift Valley Fever Virus, Rubella virus, SARS-COV-1, SARS-COV-2, Sudan Ebola virus, Vesicular Stomatitis Virus, Yellow Fever Virus, Zaire Ebola virus, and Zika Virus.

In some embodiments, the template regions are native to the virus. In some embodiments, the template regions are variants of template regions native to the virus, wherein the variants have at least 90%, 91%, 92%, 93%, 94%, 95%, 96%, 97%, 98%, or 99% sequence identity to the template regions native to the virus. In some embodiments, each of the L and the R regions of the template regions comprise fewer than 10, 9, 8, 7, 6, 5, 4, 3, or 2 variations relative to template regions native to the virus. In some embodiments, each of the L and the R regions of the template regions vary from template regions native to the virus by not more than 10, 9, 8, 7, 6, 5, 4, 3, or 2 substitutions that are not involved in 5′ capping. In some embodiments, each of the L and the R regions of the template regions varies from template regions native to the virus by not more than 1 substitution that is not involved in 5′ capping.

In some embodiments, the isolated RNA polynucleotide comprises at least one nucleoside modification. In some embodiments, the level of nucleoside modification can refer to the level of modification across the full isolated polynucleotide, or a portion thereof (e.g., the template regions). In some embodiments, the template regions are nucleoside modified, wherein the percentage of modified nucleosides is not more than 40%, 35%, 30%, 25%, 20%, 15%, 10%, or 5%. In some embodiments, the template regions are nucleoside modified, wherein the percentage of modified nucleosides is at least 30%, 35%, 40%, 45%, 50%, 55%, 60%, 65%, 70%, 75%, 80%, 90%, 95%, or 100%.

In some embodiments, the nucleoside modification is a nonimmunogenic uridine modification, and the percentage of modified uridine modifications is not more than 40%, 35%, 30%, 25%, 20% 15% or 10%. In some embodiments, the nucleoside modification is a nonimmunogenic uridine modification, and the percentage of modified uridine modifications is more than 30%, 35%, 40%, 45%, 50%, 55%, 60%, 65%, 70%, 75%, 80%, 90%, or 95%, or is 100%.

In some embodiments, the nucleoside modification is a nonimmunogenic cytidine modification, and the percentage of modified cytidine modifications is not more than 40%, 35%, 30%, 25%, 20% 15% or 10%. In some embodiments, the nucleoside modification is a nonimmunogenic cytidine modification, and the percentage of modified cytidine modifications is more than 30%, 35%, 40%, 45%, 50%, 55%, 60%, 65%, 70%, 75%, 80%, 90%, or 95%, or is 100%.

In some embodiments, the nucleoside modification is a nonimmunogenic adenosine modification, and the percentage of modified adenosine modifications is between 1% and 30%. In some embodiments, the nucleoside modification is a nonimmunogenic adenosine modification, and the percentage of modified adenosine modifications is about 1%, 5%, 10%, 15%, 20%, 25%, or 30%.

In some embodiments, the isolated polynucleotide comprises a 5′ cap structure. In some embodiments, the 5′ end of the L region comprises a 5′ cap structure. In some embodiments, the 5′ end of the L region comprises one or more variations associated with a 5′ cap structure. In some embodiments, the 5′-cap structure is selected from the group consisting of Cap 0, Cap 0 (3′-O-Me), Cap 1, Cap 1 (3′-O-Me), Cap 2, Cap 2 (3′-O-Me), Anti-Reverse Cap Analog (ARCA), inosine, N1-methyl-guanosine, 2′-fluoro-guanosine, 7-deaza-guanosine, 8-oxo-guanosine, 2-amino-guanosine, locked nucleic acid guanosine (LNA-guanosine), and 2-azido-guanosine structure.

In some embodiments, the isolated polynucleotide does not comprise a 5′ cap structure (uncapped). In some embodiments, the 5′ end of the L region does not comprise a 5′ cap structure (uncapped). In some embodiments, the 5′ end of the isolated polynucleotide comprises a 5′-monophosphate, 5′-diphosphate, or 5′-triphosphate. In some embodiments, the 5′ end of the isolated polynucleotide does not comprise a 5′-phosphate (dephosphorylated).

In some embodiments, the template regions are the reverse complement of template regions native to the virus. In some embodiments, the template regions are variants of a reverse complement of template regions native to the virus, wherein the variants have at least 90%, 91%, 92%, 93%, 94%, 95%, 96%, 97%, 98%, or 99% sequence identity to the reverse complement of the template regions native to the virus. In some embodiments, the reverse complements of each of the L and the R regions vary from the reverse complements of template regions native to the virus by not more than 10, 9, 8, 7, 6, 5, 4, 3, or 2 substitutions that are not involved in 5′ capping. In some embodiments, the reverse complements of each of the L and the R regions vary from the reverse complements of template regions native to the virus by not more than 1 substitution that is not involved in 5′ capping.

In some embodiments, the isolated RNA polynucleotide comprises at least one nucleoside modification. In some embodiments, the level of nucleoside modification can refer to the level of modification across the full isolated polynucleotide, or a portion thereof (e.g., the template regions). In some embodiments, the template regions are nucleoside-modified and the percentage of modified nucleotides is not more than 40%, 35%, 30%, 25%, 20%, 15%, 10%, or 5%. In some embodiments, the 5′ end of the reverse complement of the R region encodes a cap structure. In some embodiments, the 5′ end of the R region is capped.

In some embodiments, the therapeutic polypeptide is a secreted polypeptide. In some embodiments, the therapeutic polypeptide is selected from the group consisting of an interferon, an interferon stimulated gene, a cytokine, a chemokine, an antibody, a signaling molecule, a cytotoxic protein, a protein that causes cell death, an antineoplastic protein, an immunomodulatory protein, protein toll-like receptor agonist, or a dominant negative protein. In some embodiments, the cytokine is an inflammatory cytokine. In some embodiments, the inflammatory cytokine is TNF-α. In some embodiments, the cytokine is an anti-inflammatory cytokine. In some embodiments, the anti-inflammatory cytokine is an interleukin-1 receptor antagonist (IL-1RN). In some embodiments, the therapeutic polypeptide is an interleukin or a caspase. In some embodiments, the interleukin is IL-12A, IL-12B or IL-2. In some embodiments, the secreted protein is an antibody.

In some embodiments, the therapeutic polypeptide is an interferon. In some embodiments, the interferon is an IFN-α, IFN-β, IFN-ε, IFN-κ, IFN-ω, IFN-γ, or IFN-λ. In some embodiments, the interferon is IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-1, IFN-ε, IFN-κ, IFN-ω1, IFN-γ, IFN-λ1 (IL28A), IFN-λ2 (IL28B), IFN-λ3 (IL29), or IFN-λ4. In some embodiments, the interferon is IFN-α, IFN-β, IFN-κ, IFN-λ1 (IL28A), IFN-λ2 (IL28B), or IFN-λ3 (IL29).

In some embodiments, the coding sequence encodes more than one therapeutic polypeptide, which may be separated by one or more ribosomal skipping sequence. In some embodiments, the coding region further comprises one or more regulatory elements selected from the group consisting of ribosomal binding site, Kozak sequence, Shine-Dalgarno sequence, ribozyme, riboswitch, promoter, microRNA binding site, and internal ribosomal entry site (IRES). In some embodiments, the one or more regulatory elements are operably linked to the coding sequence. In some embodiments, the RNA polynucleotide further comprises a polyadenylation signal and/or a 3′ poly(A) tail.

In some embodiments, the RNA-dependent polymerase is an RNA-dependent RNA polymerase. In some embodiments, the RNA-dependent polymerase is an RNA-dependent DNA polymerase. In some embodiments, the RNA-dependent polymerase is a polymerase is from the virus.

In some embodiments, the isolated RNA polynucleotide is a single stranded RNA. In some embodiments, the isolated polynucleotide is in linear form. In some embodiments, the isolated polynucleotide is in a covalently-closed circular form.

In some embodiments, the virus is a sarbecovirus, wherein the L region comprises a nucleotide sequence set forth as any one of SEQ ID NOs: 60, 61, 62, 63, 64, 65, 66, or 67; or a variant of any one of SEQ ID NOs: 60, 61, 62, 63, 64, 65, 66, or 67, and wherein the R region comprises the nucleotide sequence set forth SEQ ID NO: 129; or a variant of SEQ ID NO: 129. In some embodiments, the variant comprises a variation at one or more nucleotide positions selected from the group consisting of positions 39-789 or 1426-1493 of any one of SEQ ID NOs: 60, 61, 62, 63, 64, 65, 66, or 67. In some embodiments, the variant of SEQ ID NO: 129 comprises a variation at one or more nucleotide positions selected from the group consisting of positions 20-320 of SEQ ID NO: 129.

In some embodiments, the virus is a sarbecovirus, wherein the L region comprises a nucleotide sequence set forth as any one of SEQ ID NOs: 68, 69, 70, 71, 72, 73, 74, 75, 76, or 77; or a variant of any one of SEQ ID NOW: 68, 69, 70, 71, 72, 73, 74, 75, 76, or 77, and wherein the R region comprises the nucleotide sequence set forth as SEQ ID NO: 130; or a variant of SEQ ID NO: 130. In some embodiments, (i) the variant of SEQ ID NO: 68 comprises a variation at one or more nucleotide positions selected from the group consisting of positions 39-789 or 1434-1501 of SEQ ID NO: 68; (ii) the variant of SEQ ID NO: 69 comprises a variation at one or more nucleotide positions selected from the group consisting of positions 39-789 or 1434-1501 of SEQ ID NO: 69; (iii) the variant of SEQ ID NO: 70 comprises a variation at one or more nucleotide positions selected from the group consisting of positions 39-789 or 1446-1513 of SEQ ID NO: 70; (iv) the variant of SEQ ID NO: 71 comprises a variation at one or more nucleotide positions selected from the group consisting of positions 39-789 or 1455-1522 of SEQ ID NO: 71; (v) the variant of SEQ ID NO: 72 comprises a variation at one or more nucleotide positions selected from the group consisting of positions 39-789 or 1462-1529 of SEQ ID NO: 72; (vi) the variant of SEQ ID NO: 73 comprises a variation at one or more nucleotide positions selected from the group consisting of positions 39-789 or 1469-1536 of SEQ ID NO: 73; (vii) the variant of SEQ ID NO: 74 comprises a variation at one or more nucleotide positions selected from the group consisting of positions 39-789 or 1485-1552 of SEQ ID NO: 74; (viii) the variant of SEQ ID NO: 75 comprises a variation at one or more nucleotide positions selected from the group consisting of positions 39-789 or 1686-1753 of SEQ ID NO: 75; (ix) the variant of SEQ ID NO: 76 comprises a variation at one or more nucleotide positions selected from the group consisting of positions 39-789 or 1704-1771 of SEQ ID NO: 76; or (x) the variant of SEQ ID NO: 77 comprises a variation at one or more nucleotide positions selected from the group consisting of positions 39-789 or 1720-1787 of SEQ ID NO: 77. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 78, 79, 80, 81, 82, 83, 85, 86, 87, or 88; or a variant of any one of SEQ ID NOs: 78, 79, 80, 81, 82, 83, 85, 86, 87, or 88, and wherein the R region comprises the nucleotide sequence set forth as SEQ ID NO: 130; or a variant of SEQ ID NO: 130. In some embodiments, (i) the variant of SEQ ID NO: 78 comprises a variation at one or more nucleotide positions selected from the group consisting of positions 39-789 or 1734-1801 of SEQ ID NO: 78; (ii) the variant of SEQ ID NO: 79 comprises a variation at one or more nucleotide positions selected from the group consisting of positions 39-789 or 1687-1754 of SEQ ID NO: 79; (iii) the variant of SEQ ID NO: 80 comprises a variation at one or more nucleotide positions selected from the group consisting of positions 39-789 or 1695-1762 of SEQ ID NO: 80; (iv) the variant of SEQ ID NO: 81 comprises a variation at one or more nucleotide positions selected from the group consisting of positions 39-789 or 1434-1501 of SEQ ID NO: 81; (v) the variant of SEQ ID NO: 82 comprises a variation at one or more nucleotide positions selected from the group consisting of positions 39-789 or 1443-1510 of SEQ ID NO: 82; (vi) the variant of SEQ ID NO: 83 comprises a variation at one or more nucleotide positions selected from the group consisting of positions 39-789 or 1459-1526 of SEQ ID NO: 83; (vii) the variant of SEQ ID NO: 85 comprises a variation at one or more nucleotide positions selected from the group consisting of positions 39-789 or 1434-1501 of SEQ ID NO: 85; (viii) the variant of SEQ ID NO: 86 comprises a variation at one or more nucleotide positions selected from the group consisting of positions 39-789 or 1434-1501 of SEQ ID NO: 86; (ix) the variant of SEQ ID NO: 87 comprises a variation at one or more nucleotide positions selected from the group consisting of positions 39-789 or 1435-1502 of SEQ ID NO: 87; or (x) the variant of SEQ ID NO: 88 comprises a variation at one or more nucleotide positions selected from the group consisting of positions 39-789 or 1463-1530 of SEQ ID NO: 88. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 89, 90, 91, 92, 96, 104, 105, 106, 107, or 108; or a variant of any one of SEQ ID NOs: 89, 90, 91, 92, 96, 104, 105, 106, 107, or 108, and wherein the R region comprises the nucleotide sequence set forth as SEQ ID NO: 130; or a variant of SEQ ID NO: 130. In some embodiments, (i) the variant of SEQ ID NO: 89 comprises a variation at one or more nucleotide positions selected from the group consisting of positions 39-789 or 1466-1533 of SEQ ID NO: 89; (ii) the variant of SEQ ID NO: 90 comprises a variation at one or more nucleotide positions selected from the group consisting of positions 39-789 or 1425-1492 of SEQ ID NO: 90; (iii) the variant of SEQ ID NO: 91 comprises a variation at one or more nucleotide positions selected from the group consisting of positions 39-789 or 1425-1492 of SEQ ID NO: 91; (iv) the variant of SEQ ID NO: 92 comprises a variation at one or more nucleotide positions selected from the group consisting of positions 39-789 or 1425-1492 of SEQ ID NO: 92; (v) the variant of SEQ ID NO: 96 comprises a variation at one or more nucleotide positions selected from the group consisting of positions 39-769 or 1471-1471 of SEQ ID NO: 96; (vi) the variant of SEQ ID NO: 104 comprises a variation at one or more nucleotide positions selected from the group consisting of positions 39-789 or 1446-1513 of SEQ ID NO: 104; (vii) the variant of SEQ ID NO: 105 comprises a variation at one or more nucleotide positions selected from the group consisting of positions 39-789 or 1455-1522 of SEQ ID NO: 105; (viii) the variant of SEQ ID NO: 106 comprises a variation at one or more nucleotide positions selected from the group consisting of positions 39-789 or 1462-1529 of SEQ ID NO: 106; (ix) the variant of SEQ ID NO: 107 comprises a variation at one or more nucleotide positions selected from the group consisting of positions 39-789 or 1469-1536 of SEQ ID NO: 107; or (x) the variant of SEQ ID NO: 108 comprises a variation at one or more nucleotide positions selected from the group consisting of positions 89-839 or 1485-1552 of SEQ ID NO: 108. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 109, 110, 111, 112, 113, 114, 115, 116, 117, or 118; or a variant of any one of SEQ ID NOs: 109, 110, 111, 112, 113, 114, 115, 116, 117, or 118, and wherein the R region comprises the nucleotide sequence set forth as SEQ ID NO: 130; or a variant of SEQ ID NO: 130. In some embodiments, (i) the variant of SEQ ID NO: 109 comprises a variation at one or more nucleotide positions selected from the group consisting of positions 40-789 or 1686-1753 of SEQ ID NO: 109; (ii) the variant of SEQ ID NO: 110 comprises a variation at one or more nucleotide positions selected from the group consisting of positions 40-789 or 1704-1771 of SEQ ID NO: 110; (iii) the variant of SEQ ID NO: 111 comprises a variation at one or more nucleotide positions selected from the group consisting of positions 40-789 or 1720-1787 of SEQ ID NO: 111; (iv) the variant of SEQ ID NO: 112 comprises a variation at one or more nucleotide positions selected from the group consisting of positions 40-789 or 1734-1801 of SEQ ID NO: 112; (v) the variant of SEQ ID NO: 113 comprises a variation at one or more nucleotide positions selected from the group consisting of positions 40-789 or 1687-1754 of SEQ ID NO: 113; (vi) the variant of SEQ ID NO: 114 comprises a variation at one or more nucleotide positions selected from the group consisting of positions 40-789 or 1695-1762 of SEQ ID NO: 114; (vii) the variant of SEQ ID NO: 115 comprises a variation at one or more nucleotide positions selected from the group consisting of positions 40-789 or 1434-1501 of SEQ ID NO: 115; (viii) the variant of SEQ ID NO: 116 comprises a variation at one or more nucleotide positions selected from the group consisting of positions 40-789 or 1434-1501 of SEQ ID NO: 116; (ix) the variant of SEQ ID NO: 117 comprises a variation at one or more nucleotide positions selected from the group consisting of positions 40-789 or 1434-1501 of SEQ ID NO: 117; or (xl) the variant of SEQ ID NO: 118 comprises a variation at one or more nucleotide positions selected from the group consisting of positions 40-789 or 1434-1501 of SEQ ID NO: 118. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 119, 120, 122, 123, 124, 125, 126, or 127; or a variant of any one of SEQ ID NOs: 119, 120, 122, 123, 124, 125, 126, or 127; and wherein the R region comprises the nucleotide sequence set forth as SEQ ID NO: 130; or a variant of SEQ ID NO: 130. In some embodiments, (i) the variant of SEQ ID NO: 119 comprises a variation at one or more nucleotide positions selected from the group consisting of positions 40-789 or 1443-1510 of SEQ ID NO: 119; (ii) the variant of SEQ ID NO: 120 comprises a variation at one or more nucleotide positions selected from the group consisting of positions 40-789 or 1459-1526 of SEQ ID NO: 120; (iii) the variant of SEQ ID NO: 122 comprises a variation at one or more nucleotide positions selected from the group consisting of positions 40-789 or 1434-1501 of SEQ ID NO: 122; (iv) the variant of SEQ ID NO: 123 comprises a variation at one or more nucleotide positions selected from the group consisting of positions 40-789 or 1434-1501 of SEQ ID NO: 123; (v) the variant of SEQ ID NO: 124 comprises a variation at one or more nucleotide positions selected from the group consisting of positions 40-789 or 1434-1501 of SEQ ID NO: 124; (vi) the variant of SEQ ID NO: 125 comprises a variation at one or more nucleotide positions selected from the group consisting of positions 40-789 or 1463-1530 of SEQ ID NO: 125; (vii) the variant of SEQ ID NO: 126 comprises a variation at one or more nucleotide positions selected from the group consisting of positions 40-789 or 1466-1533 of SEQ ID NO: 126; (viii) the variant of SEQ ID NO: 127 comprises a variation at one or more nucleotide positions selected from the group consisting of positions 40-789 or 1425-1492 of SEQ ID NO: 127. In some embodiments, the variant of SEQ ID NO: 130 comprises a variation at one or more nucleotide positions selected from the group consisting of positions 20-320 of SEQ ID NO: 130.

In some embodiments, the virus is a Respiratory Syncytial Virus (RSV), wherein the L region comprises a nucleotide sequence set forth as any one of SEQ ID NOs: 148, 149, 150, 151, or 152; or a variant of any one of SEQ ID NOs: 148, 149, 150, 151, or 152, and wherein the R region comprises the nucleotide sequence set forth as SEQ ID NO: 154 or 155. In some embodiments, (i) the variant of SEQ ID NO: 148 comprises a variation at one or more nucleotide positions selected from the group consisting of positions 15-78 of SEQ ID NO: 148; (ii) the variant of SEQ ID NO: 149 comprises a variation at one or more nucleotide positions selected from the group consisting of positions 15-33 of SEQ ID NO: 149; (iii) the variant of SEQ ID NO: 150 comprises a variation at one or more nucleotide positions selected from the group consisting of positions 15-35 of SEQ ID NO: 150; (iv) the variant of SEQ ID NO: 151 comprises a variation at one or more nucleotide positions selected from the group consisting of positions 18-36 of SEQ ID NO: 151; or (v) the variant of SEQ ID NO: 152 comprises a variation at one or more nucleotide positions selected from the group consisting of positions 15-38 of SEQ ID NO: 152. In some embodiments, (i) the variant of SEQ ID NO: 154 comprises a variation at one or more nucleotide positions selected from the group consisting of positions 15-207 of SEQ ID NO: 154; or (ii) the variant of SEQ ID NO: 155 comprises a variation at one or more nucleotide positions selected from the group consisting of positions 15-32 of SEQ ID NO: 155.

In some embodiments, the virus is a parainfluenzavirus, wherein the L region comprises a nucleotide sequence set forth as SEQ ID NO: 180; or a variant of SEQ ID NO: 180, and wherein the R region comprises the nucleotide sequence set forth as SEQ ID NO: 179; or a variant of SEQ ID NO: 179. In some embodiments, the variant of SEQ ID NO: 180 comprises a variation at one or more nucleotide positions selected from the group consisting of positions 17-136 of SEQ ID NO: 180. In some embodiments, the variant of SEQ ID NO: 179 comprises a variation at one or more nucleotide positions selected from the group consisting of positions 17-98 of SEQ ID NO: 179. In some embodiments, the virus is a parainfluenzavirus, wherein the L region comprises a nucleotide sequence set forth as SEQ ID NO: 186; or a variant of SEQ ID NO: 186, and wherein the R region comprises the nucleotide sequence set forth as SEQ ID NO: 185; or a variant of SEQ ID NO: 185. In some embodiments, the variant of SEQ ID NO: 186 comprises a variation at one or more nucleotide positions selected from the group consisting of positions 21-95 of SEQ ID NO: 186. In some embodiments, the variant of SEQ ID NO: 185 comprises a variation at one or more nucleotide positions selected from the group consisting of positions 21-93 of SEQ ID NO: 185.

In some embodiments, the virus is a metapneumovirus, wherein the L region comprises a nucleotide sequence set forth as SEQ ID NO: 194; or a variant of SEQ ID NO: 194, and wherein the R region comprises the nucleotide sequence set forth as SEQ ID NO: 192; or a variant of any one of SEQ ID NO: 192. In some embodiments, the variant of SEQ ID NO: 194 comprises a variation at one or more nucleotide positions selected from the group consisting of positions 17-220 of SEQ ID NO: 194. In some embodiments, the variant of SEQ ID NO: 192 comprises a variation at one or more nucleotide positions selected from the group consisting of positions 17-32 of SEQ ID NO: 192. In some embodiments, the virus is a metapneumovirus, wherein the L region comprises a nucleotide sequence set forth as SEQ ID NO: 193; or a variant of SEQ ID NO: 193, and wherein the R region comprises the nucleotide sequence set forth as SEQ ID NO: 191; or a variant of SEQ ID NO: 191. In some embodiments, the variant of SEQ ID NO: 193 comprises a variation at one or more nucleotide positions selected from the group consisting of positions 17-220 of SEQ ID NO: 193. In some embodiments, the variant of SEQ ID NO: 191 comprises a variation at one or more nucleotide positions selected from the group consisting of positions 17-32 of SEQ ID NO: 191.

In some embodiments, the virus is a henipavirus, wherein the L region comprises a nucleotide sequence set forth as SEQ ID NO: 203; or a variant of SEQ ID NO: 203, and wherein the R region comprises the nucleotide sequence set forth as SEQ ID NO: 202; or a variant of SEQ ID NO: 202. In some embodiments, the variant of SEQ ID NO: 203 comprises a variation at one or more nucleotide positions selected from the group consisting of positions 17-77 of SEQ ID NO: 203. In some embodiments, the variant of SEQ ID NO: 202 comprises a variation at one or more nucleotide positions selected from the group consisting of positions 17-91 of SEQ ID NO: 202. In some embodiments, the virus is a henipavirus, wherein the L region comprises a nucleotide sequence set forth as SEQ ID NO: 207; or a variant of SEQ ID NO: 207, and wherein the R region comprises the nucleotide sequence set forth as SEQ ID NO: 208; or a variant of SEQ ID NO: 208. In some embodiments, the variant of SEQ ID NO: 207 comprises a variation at one or more nucleotide positions selected from the group consisting of positions 17-77 of SEQ ID NO: 207. In some embodiments, the variant of SEQ ID NO: 208 comprises a variation at one or more nucleotide positions selected from the group consisting of positions 17-91 of SEQ ID NO: 208.

In some embodiments, the virus is a hepadnavirus, wherein the L region comprises a nucleotide sequence set forth as any one of SEQ ID NOs: 212, 213, 214, 215, or 216; or a variant of any one of SEQ ID NOs: 212, 213, 214, 215, or 216, and wherein the R region comprises the nucleotide sequence set forth as any one of SEQ ID NOs: 217, 218, 219, or 220; or a variant of any one of SEQ ID NOs: 217, 218, 219, or 220. In some embodiments, (i) the variant of SEQ ID NO: 212 comprises a variation at one or more nucleotide positions selected from the group consisting of positions 101-1326 of SEQ ID NO: 212; (ii) the variant of SEQ ID NO: 213 comprises a variation at one or more nucleotide positions selected from the group consisting of positions 101-1291 of SEQ ID NO: 213; (iii) the variant of SEQ ID NO: 214 comprises a variation at one or more nucleotide positions selected from the group consisting of positions 101-1325 of SEQ ID NO: 214; (iv) the variant of SEQ ID NO: 215 comprises a variation at one or more nucleotide positions selected from the group consisting of positions 101-15 of SEQ ID NO: 215; or (v) the variant of SEQ ID NO: 216 comprises a variation at one or more nucleotide positions selected from the group consisting of positions 101-211 of SEQ ID NO: 216. In some embodiments, (i) the variant of SEQ ID NO: 217 comprises a variation at one or more nucleotide positions selected from the group consisting of positions 101-754 of SEQ ID NO: 217; (ii) the variant of SEQ ID NO: 218 comprises a variation at one or more nucleotide positions selected from the group consisting of positions 101-790 of SEQ ID NO: 218; (iii) the variant of SEQ ID NO: 219 comprises a variation at one or more nucleotide positions selected from the group consisting of positions 101-892 of SEQ ID NO: 219; or (iv) the variant of SEQ ID NO: 220 comprises a variation at one or more nucleotide positions selected from the group consisting of positions 101-2309 of SEQ ID NO: 220.

In some embodiments, the virus is an alphavirus, wherein the L region comprises a nucleotide sequence set forth as any one of SEQ ID NO: 252 or 253; or a variant of any one of SEQ ID NO: 252 or 253, and wherein the R region comprises the nucleotide sequence set forth as any one of SEQ ID NO: 254; or a variant of any one of SEQ ID NO: 254. In some embodiments, (i) the variant of SEQ ID NO: 252 comprises a variation at one or more nucleotide positions selected from the group consisting of positions 100-223 of SEQ ID NO: 252; or (ii) the variant of SEQ ID NO: 253 comprises a variation at one or more nucleotide positions selected from the group consisting of positions 100-415 of SEQ ID NO: 253. In some embodiments, (i) the variant of SEQ ID NO: 254 comprises a variation at one or more nucleotide positions selected from the group consisting of positions 100-321 of SEQ ID NO: 254. In some embodiments, the virus is an alphavirus, wherein the L region comprises a nucleotide sequence set forth as any one of SEQ ID NO: 258 or 259; or a variant of any one of SEQ ID NO: 258 or 259, and wherein the R region comprises the nucleotide sequence set forth as any one of SEQ ID NO: 260; or a variant of any one of SEQ ID NO: 260. In some embodiments, (i) the variant of SEQ ID NO: 258 comprises a variation at one or more nucleotide positions selected from the group consisting of positions 100-323 of SEQ ID NO: 258; or (ii) the variant of SEQ ID NO: 259 comprises a variation at one or more nucleotide positions selected from the group consisting of positions 100-427 of SEQ ID NO: 259. In some embodiments, the variant of SEQ ID NO: 260 comprises a variation at one or more nucleotide positions selected from the group consisting of positions 60-84 of SEQ ID NO: 260.

In some embodiments, the virus is an alphavirus, wherein the L region comprises a nucleotide sequence set forth as any one of SEQ ID NO: 264 or 265; or a variant of any one of SEQ ID NO: 264 or 265, and wherein the R region comprises the nucleotide sequence set forth as any one of SEQ ID NO: 266; or a variant of any one of SEQ ID NO: 266. In some embodiments, (i) the variant of SEQ ID NO: 264 comprises a variation at one or more nucleotide positions selected from the group consisting of positions 100-216 of SEQ ID NO: 264; or (ii) the variant of SEQ ID NO: 265 comprises a variation at one or more nucleotide positions selected from the group consisting of positions 100-429 of SEQ ID NO: 265. In some embodiments, the variant of SEQ ID NO: 266 comprises a variation at one or more nucleotide positions selected from the group consisting of positions 60-262 of SEQ ID NO: 266.

Aspects of the present disclosure provide isolated RNA polynucleotides comprising a coding region having a coding sequence encoding one or more polypeptides; and template regions, wherein the template regions comprise two distinct regions, a left flanking region (“L region”) of a virus and a right flanking region (“R region”) of the virus, wherein the L region is adjacent to and contiguous with a 5′ end of the coding region and the R region is adjacent to and contiguous with a 3′ end of the coding region; wherein at least 30% of uridine nucleotides are modified, at least 30% of cytidine nucleotides are modified, and/or between 1-30% of adenosine nucleotides are modified; and wherein the template region interact with and initiate RNA-dependent polymerase activity of a polymerase in a cell containing the RNA dependent polymerase.

In some embodiments, the coding sequence is in an antisense orientation. In some embodiments, the coding sequence is in a sense orientation.

In some embodiments, polypeptide is a secreted protein. In some embodiments, the polypeptide is selected from the group consisting of a medicament, a therapeutic polypeptide, an antigen, and a reporter.

Also provided herein is the reverse complement of any of the isolated RNA polynucleotides described herein.

In some embodiments, the virus is selected from the group consisting of viruses in the orders of Amarillovirales, Articulavirales, Blubervirales, Bunyavirales, Hepelivirales, Martellivirales, Mononegavirales, Nidovirales, and Picornavirales. In some embodiments, the virus is selected from the group consisting of viruses in the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, Rhabdoviridae, and Togaviridae. In some embodiments, the virus is selected from the group consisting of Alphacoronavirus 229E, Alphacoronavirus NL63, Alphacoronavirus WA2028, Avian metapneumovirus (AMPV), Betacoronavirus HKU1, Betacoronavirus HKU15, Betacoronavirus HKU33, Betacoronavirus OC43, Chikungunya virus, Crimean-Congo Hemorrhagic Fever Virus, Dengue Virus, Eastern Equine Encephalitis Virus (EEEV), Enterovirus D68 (EV-D68), Foot and Mouth Disease Virus, Hanta Virus, Hendra Virus, Hepatitis B Virus, Hepatitis C Virus, HMPV, Human Parainfluenzavirus 1 (HPIV1), Human Parainfluenzavirus 3 (HPIV3), Infectious Salmon Anemia Virus, Influenza A Virus, Influenza B Virus, Lassa Virus, Marburg Virus, Middle East Respiratory Syndrome Coronavirus (MERS-CoV), Newcastle Disease Virus (NDV), Nipah Virus, Norwalk Virus, Rabies Virus, Respiratory Syncytial Virus, Reston Ebola virus, Rhinovirus, Rift Valley Fever Virus, Rubella virus, SARS-COV-1, SARS-COV-2, Sudan Ebola virus, Venezuelan Equine Encephalitis Virus (VEEV), Vesicular Stomatitis Virus, Western Equine Encephalitis Virus (WEEV), Yellow Fever Virus, Zaire Ebola virus, and Zika Virus.

In some embodiments, the template regions are native to the virus. In some embodiments, the template regions are variants of template regions native to the virus, wherein the variant has at least 90%, 91%, 92%, 93%, 94%, 95%, 96%, 97%, 98%, or 99% sequence identity to the template region native to the virus. In some embodiments, each of the L and the R regions of the template regions comprise fewer than 10, 9, 8, 7, 6, 5, 4, 3, or 2 variations relative to template regions native to the virus. In some embodiments, each of the L and the R regions of the template regions vary from template regions native to the virus by not more than 10, 9, 8, 7, 6, 5, 4, 3, or 2 substitutions that are not involved in 5′ capping. In some embodiments, each of the L and the R regions of the template regions varies from template regions native to the virus by not more than 1 substitution that is not involved in 5′ capping.

In some embodiments, the template regions are nucleoside modified, wherein the percentage of modified nucleosides is not more than 40%, 35%, 30%, 25%, 20%, 15%, 10%, or 5%. In some embodiments, the template regions are nucleoside modified, wherein the percentage of modified nucleosides at least 30%, 35%, 40%, 45%, 50%, 55%, 60%, 65%, 70%, 75%, 80%, 90%, 95%, or 100%.

In some embodiments, the nucleoside modification is a nonimmunogenic uridine modification, and the percentage of modified uridine modifications is more than 30%, 35%, 40%, 45%, 50%, 55%, 60%, 65%, 70%, 75%, 80%, 90%, or 95%, or is 100%. In some embodiments, the nucleoside modification is a nonimmunogenic cytidine modification, and the percentage of modified cytidine modifications is more than 30%, 35%, 40%, 45%, 50%, 55%, 60%, 65%, 70%, 75%, 80%, 90%, or 95%, or is 100%.

In some embodiments, the nucleoside modification is a nonimmunogenic adenosine modification, and the percentage of modified adenosine modifications is about 1%, 5%, 10%, 15%, 20%, 25%, or 30%.

In some embodiments, the isolated polynucleotide comprises a 5′ cap structure. In some embodiments, the 5′ end of the L region comprises a 5′ cap structure. In some embodiments, the 5′ end of the L region comprises one or more variations associated with a 5′ cap structure. In some embodiments, the 5′-cap structure is selected from the group consisting of Cap 0, Cap 0 (3′-O-Me), Cap 1, Cap 1 (3′-O-Me), Cap 2, Cap 2 (3′-O-Me), Anti-Reverse Cap Analog (ARCA), inosine, N1-methyl-guanosine, 2′-fluoro-guanosine, 7-deaza-guanosine, 8-oxo-guanosine, 2-amino-guanosine, locked nucleic acid guanosine (LNA-guanosine), and 2-azido-guanosine structure.

In some embodiments, the isolated polynucleotide does not comprise a 5′ cap structure (uncapped). In some embodiments, the 5′ end of the L region does not comprise a 5′ cap structure (uncapped). In some embodiments, the 5′ end of the isolated polynucleotide comprises a 5′-monophosphate, 5′-diphosphate, or 5′-triphosphate. In some embodiments, the 5′ end of the isolated polynucleotide does not comprise a 5′-phosphate (dephosphorylated).

In some embodiments, the template regions are the reverse complement of template regions native to the virus. In some embodiments, the template regions are variants of a reverse complement of template regions native to the virus, wherein the variants have at least 90%, 91%, 92%, 93%, 94%, 95%, 96%, 97%, 98%, or 99% sequence identity to the reverse complement of the template regions native to the virus. In some embodiments, the reverse complements of each of the L and the R regions vary from the reverse complements of template regions native to the virus by not more than 10, 9, 8, 7, 6, 5, 4, 3, or 2 substitutions that are not involved in 5′ capping. In some embodiments, the reverse complements of each of the L and the R regions vary from the reverse complements of template regions native to the virus by not more than 1 substitution that is not involved in 5′ capping. In some embodiments, the 5′ end of the reverse complement of the R region encodes a 5′ cap structure. In some embodiments, the 5′ end of the R region is capped.

In some embodiments, the coding sequence encodes more than one polypeptide, which may be separated by one or more ribosomal skipping sequence. In some embodiments, the coding region further comprises one or more regulatory elements selected from the group consisting of ribosomal binding site, Kozak sequence, Shine-Dalgarno sequence, ribozyme, riboswitch, promoter, microRNA binding site, and internal ribosomal entry site (IRES). In some embodiments, the one or more regulatory elements are operably linked to the coding sequence. In some embodiments, the RNA polynucleotides further comprise a polyadenylation signal and/or a 3′ poly(A) tail.

In some embodiments, the RNA-dependent polymerase is an RNA-dependent RNA polymerase. In some embodiments, the RNA-dependent polymerase is an RNA-dependent DNA polymerase. In some embodiments, the RNA-dependent polymerase is a polymerase is from the virus.

In some embodiments, the isolated RNA polynucleotide is a single stranded RNA. In some embodiments, the isolated polynucleotide is in linear form. In some embodiments, the isolated polynucleotide is in a covalently-closed circular form.

In some embodiments, the virus is an influenza virus, wherein the L region comprises a nucleotide sequence set forth as SEQ ID NO: 2; or a variant of SEQ ID NO: 2, and wherein the R region comprises the nucleotide sequence set forth as any one of SEQ ID NOs: 20, 21, 22, or 23; or a variant of any one of SEQ ID NOs: 20, 21, 22, or 23. In some embodiments, the variant of SEQ ID NO: 2 comprises a variation at one or more nucleotide positions selected from the group consisting of positions 14-26 of SEQ ID NO: 2. In some embodiments, the variant comprises a variation at one or more nucleotide positions selected from the group consisting of positions 8-15 of any one of SEQ ID NOs: 20, 21, 22, or 23. In some embodiments, the virus is an influenza virus, wherein the L region comprises a nucleotide sequence set forth as SEQ ID NO: 3; or a variant of SEQ ID NO: 3, and wherein the R region comprises the nucleotide sequence set forth as any one of SEQ ID NOs: 24, 25, 26, or 27. In some embodiments, (i) the variant of SEQ ID NO: 3 comprises a variation at one or more nucleotide positions selected from the group consisting of positions 14-35 of SEQ ID NO: 3. In some embodiments, (i) the variant of SEQ ID NO: 24 comprises a variation at one or more nucleotide positions selected from the group consisting of positions 8-12 of SEQ ID NO: 24; (ii) the variant of SEQ ID NO: 25 comprises a variation at one or more nucleotide positions selected from the group consisting of positions 8-12 of SEQ ID NO: 25; (iii) the variant of SEQ ID NO: 26 comprises a variation at one or more nucleotide positions selected from the group consisting of positions 8-12 of SEQ ID NO: 26; or (iv) the variant of SEQ ID NO: 27 comprises a variation at one or more nucleotide positions selected from the group consisting of positions 8-12 of SEQ ID NO: 27. In some embodiments, the virus is an influenza virus, wherein the L region comprises a nucleotide sequence set forth as SEQ ID NO: 4; or a variant of SEQ ID NO: 4, and wherein the R region comprises the nucleotide sequence set forth as any one of SEQ ID NOs: 28, 29, 30, or 31; or a variant of any one of SEQ ID NOs: 28, 29, 30, or 31. In some embodiments, the variant of SEQ ID NO: 4 comprises a variation at one or more nucleotide positions selected from the group consisting of positions 14-50 of SEQ ID NO: 4. In some embodiments, the variant of any one of SEQ ID NOs: 28, 29, 30, or 31 comprises a variation at one or more nucleotide positions selected from the group consisting of positions 8-12 of any one of SEQ ID NOs: 28, 29, 30, or 31.

In some embodiments, the virus is an influenza virus, wherein the L region comprises a nucleotide sequence set forth as SEQ ID NO: 1 or 5; or a variant of SEQ ID NO: 1 or 5, and wherein the R region comprises the nucleotide sequence set forth as SEQ ID NO: 18 or 19; or a variant of SEQ ID NO: 18 or 19. In some embodiments, the variant of SEQ ID NO: 1 or 5 comprises a variation at one or more nucleotide positions selected from the group consisting of positions 14-37 of SEQ ID NO: 1 or 5. In some embodiments, the variant of SEQ ID NO: 18 or 19 comprises a variation at one or more nucleotide positions selected from the group consisting of positions 8-20 of SEQ ID NO: 18 or 19. In some embodiments, the virus is an influenza virus, wherein the L region comprises a nucleotide sequence set forth as SEQ ID NO: 6; or a variant of SEQ ID NO: 6, and wherein the R region comprises the nucleotide sequence set forth as SEQ ID NO: 32 or 33; or a variant of SEQ ID NO: 32 or 33. In some embodiments, the variant of SEQ ID NO: 6 comprises a variation at one or more nucleotide positions selected from position 14 or 15 of SEQ ID NO: 6. In some embodiments, the variant of SEQ ID NO: 32 or 33 comprises a variation at one or more nucleotide positions selected from the group consisting of positions 8-33 of SEQ ID NO: 32 or 33. In some embodiments, the virus is an influenza virus, wherein the L region comprises a nucleotide sequence set forth as SEQ ID NO: 7; or a variant of SEQ ID NO: 7, and wherein the R region comprises the nucleotide sequence set forth as any one of SEQ ID NOs: 34, 35, 36, or 37; or a variant of any one of SEQ ID NOs: 34, 35, 36, or 37. In some embodiments, the variant of SEQ ID NO: 7 comprises a variation at one or more nucleotide positions selected from the group consisting of positions 14-20 of SEQ ID NO: 7. In some embodiments, the variant of any one of SEQ ID NOs: 34, 35, 36, or 37, wherein the variant comprises a variation at one or more nucleotide positions selected from the group consisting of positions 5-8 of SEQ ID NO: 34, 35, 36, or 37. In some embodiments, the virus is an influenza virus, wherein the L region comprises a nucleotide sequence set forth as SEQ ID NO: 8; or a variant of SEQ ID NO: 8 and wherein the R region comprises the nucleotide sequence set forth as any one of SEQ ID NOs: 38, 39, 40, or 41; or a variant of any one of SEQ ID NOs: 38, 39, 40, or 41. In some embodiments, the variant of SEQ ID NO: 8 comprises a variation at one or more nucleotide positions selected from position 14 or 15 of SEQ ID NO: 8. In some embodiments, the variant of any one of SEQ ID NOs: 38, 39, 40, or 41 comprises a variation at one or more nucleotide positions selected from the group consisting of positions 8-13 of SEQ ID NO: 38, 39, 40, or 41. In some embodiments, the virus is an influenza virus, wherein the L region comprises a nucleotide sequence set forth as SEQ ID NO: 9; or a variant of SEQ ID NO: 9, and wherein the R region comprises the nucleotide sequence set forth as SEQ ID NO: 42 or 43; or a variant of SEQ ID NO: 42 or 43. In some embodiments, the variant of SEQ ID NO: 9 comprises a variation at one or more nucleotide positions selected from the group consisting of positions 14-18 of SEQ ID NO: 9. In some embodiments, the variant of SEQ ID NO: 42 or 43 comprises a variation at one or more nucleotide positions selected from the group consisting of positions 8-14 of SEQ ID NO: 42 or 43. In some embodiments, the virus is an influenza virus, wherein the L region comprises a nucleotide sequence set forth as SEQ ID NO: 11; or a variant of SEQ ID NO: 11, and wherein the R region comprises the nucleotide sequence set forth as SEQ ID NO: 46 or 47; or a variant of SEQ ID NO: 46 or 47. In some embodiments, the variant of SEQ ID NO: 11 comprises a variation at one or more nucleotide positions selected from the group consisting of positions 14-81 of SEQ ID NO: 11. In some embodiments, the variant of SEQ ID NO: 46 or 47t comprises a variation at one or more nucleotide positions selected from the group consisting of positions 5-9 of SEQ ID NO: 46 or 47. In some embodiments, the virus is an influenza virus, wherein the L region comprises a nucleotide sequence set forth as SEQ ID NO: 12; or a variant of SEQ ID NO: 12, and wherein the R region comprises the nucleotide sequence set forth as any one of SEQ ID NO: 48 or 49; or a variant of any one of SEQ ID NO: 48 or 49. In some embodiments, the variant of SEQ ID NO: 12 comprises a variation at one or more nucleotide positions selected from the group consisting of positions 14-52 of SEQ ID NO: 12. In some embodiments, the variant of any one of SEQ ID NO: 48 or 49 comprises a variation at one or more nucleotide positions selected from the group consisting of positions 8-11 of SEQ ID NO: 48 or 49. In some embodiments, the virus is an influenza virus, wherein the L region comprises a nucleotide sequence set forth as SEQ ID NO: 13; or a variant of SEQ ID NO: 13 and wherein the R region comprises the nucleotide sequence set forth as SEQ ID NO: 50 or 51; or a variant of SEQ ID NO: 50 or 51. In some embodiments, the variant of SEQ ID NO: 13 comprises a variation at one or more nucleotide positions selected from the group consisting of positions 14-87 of SEQ ID NO: 13. In some embodiments, the variant of SEQ ID NO: 50 or 51 comprises a variation at one or more nucleotide positions selected from the group consisting of positions 8-17 of SEQ ID NO: 50 or 51. In some embodiments, the virus is an influenza virus, wherein the L region comprises a nucleotide sequence set forth as any one of SEQ ID NO: 10; or a variant of SEQ ID NO: 10, and wherein the R region comprises the nucleotide sequence set forth as SEQ ID NO: 44 or 45; or a variant of SEQ ID NO: 44 or 45. In some embodiments, the variant of SEQ ID NO: 10 comprises a variation at one or more nucleotide positions selected from the group consisting of positions 14-86 of SEQ ID NO: 10. In some embodiments, the variant of SEQ ID NO: 44 or 45 comprises a variation at one or more nucleotide positions selected from the group consisting of positions 8-21 of SEQ ID NO: 44 or 45. In some embodiments, the virus is an influenza virus, wherein the L region comprises a nucleotide sequence set forth as SEQ ID NO: 14; or a variant of SEQ ID NO: 14, and wherein the R region comprises the nucleotide sequence set forth as any one of SEQ ID NO: 52 or 53; or a variant of any one of SEQ ID NO: 52 or 53. In some embodiments, the variant of SEQ ID NO: 14 comprises a variation at one or more nucleotide positions selected from the group consisting of positions 14-93 of SEQ ID NO: 14. In some embodiments, the variant of any one of SEQ ID NO: 52 or 53 comprises a variation at one or more nucleotide positions selected from the group consisting of positions 8-48 of SEQ ID NO: 52 or 53. In some embodiments, the virus is an influenza virus, wherein the L region comprises a nucleotide sequence set forth as SEQ ID NO: 15; or a variant of SEQ ID NO: 15, and wherein the R region comprises the nucleotide sequence set forth as SEQ ID NO: 54 or 55; or a variant of any one of SEQ ID NO: 54 or 55. In some embodiments, the variant of SEQ ID NO: 15 comprises a variation at one or more nucleotide positions selected from the group consisting of positions 14-95 of SEQ ID NO: 15. In some embodiments, the variant of any one of SEQ ID NO: 54 or 55 comprises a variation at one or more nucleotide positions selected from the group consisting of positions 8-34 of SEQ ID NO: 54 or 55.

In some embodiments, the virus is an influenza virus, wherein the L region comprises a nucleotide sequence set forth as SEQ ID NO: 16; or a variant of SEQ ID NO: 16, and wherein the R region comprises the nucleotide sequence set forth as any one of SEQ ID NO: 56 or 57; or a variant of any one of SEQ ID NO: 56 or 57. In some embodiments, the variant of SEQ ID NO: 16 comprises a variation at one or more nucleotide positions selected from the group consisting of positions 14-81 of SEQ ID NO: 16. In some embodiments; the variant of any one of SEQ ID NO: 56 or 57 comprises a variation at one or more nucleotide positions selected from the group consisting of positions 8-12 of SEQ ID NO: 56 or 57. In some embodiments, the virus is an influenza virus, wherein the L region comprises a nucleotide sequence set forth as SEQ ID NO: 17; or a variant of SEQ ID NO: 17, and wherein the R region comprises the nucleotide sequence set forth as any one of SEQ ID NO: 58 or 59; or a variant of any one of SEQ ID NO: 58 or 59. In some embodiments, the variant of SEQ ID NO: 17 comprises a variation at one or more nucleotide positions selected from the group consisting of positions 14-22 of SEQ ID NO: 17 In some embodiments, the variant of any one of SEQ ID NO: 58 or 59 comprises a variation at one or more nucleotide positions selected from the group consisting of positions 8-32 of SEQ ID NO: 58 or 59.

In some embodiments, the virus is a sarbecovirus, wherein the L region comprises a nucleotide sequence set forth as SEQ ID NO: 137; or a variant of SEQ ID NO: 137, and wherein the R region comprises the nucleotide sequence set forth as SEQ ID NO: 128; or a variant of any one of SEQ ID NO: 128. In some embodiments, the variant of SEQ ID NO: 137 comprises a variation at one or more nucleotide positions selected from the group consisting of positions 40-1557 of SEQ ID NO: 137. In some embodiments, the variant of any one of SEQ ID NO: 128 comprises a variation at one or more nucleotide positions selected from the group consisting of positions 20-30 of SEQ ID NO: 128. In some embodiments, the virus is a sarbecovirus, wherein the L region comprises a nucleotide sequence set forth as any one of SEQ ID NOs: 138, 139, 140, 141, 142, 143, or 144; or a variant of any one of SEQ ID NOs: 138, 139, 140, 141, 142, 143, or 144, and wherein the R region comprises the nucleotide sequence set forth as any one of SEQ ID NOs: 130, 136, 145, 146, or 147; or a variant of any one of SEQ ID NOs: 130, 136, 145, 146, or 147. In some embodiments, (i) the variant of SEQ ID NO: 138 comprises a variation at one or more nucleotide positions selected from the group consisting of positions 50-312 of SEQ ID NO: 138; (ii) the variant of SEQ ID NO: 139 comprises a variation at one or more nucleotide positions selected from the group consisting of positions 50-1567 of SEQ ID NO: 139; (iii) the variant of SEQ ID NO: 140 comprises a variation at one or more nucleotide positions selected from the group consisting of positions 50-1488 of SEQ ID NO: 140; (iv) the variant of SEQ ID NO: 141 comprises a variation at one or more nucleotide positions selected from the group consisting of positions 50-1593 of SEQ ID NO: 141; (v) the variant of SEQ ID NO: 142 comprises a variation at one or more nucleotide positions selected from the group consisting of positions 50-1570 of SEQ ID NO: 142; (vi) the variant of SEQ ID NO: 143 comprises a variation at one or more nucleotide positions selected from the group consisting of positions 50-1488 of SEQ ID NO: 143; or (vii) the variant of SEQ ID NO: 144 comprises a variation at one or more nucleotide positions selected from the group consisting of positions 50-1593 of SEQ ID NO: 144. In some embodiments, the variant of any one of SEQ ID NOs: 130, 136, 145, 146, or 147 comprises a variation at one or more nucleotide positions selected from the group consisting of positions 20-320 of SEQ ID NO: 130; (ii) the variant of SEQ ID NO: 136 comprises a variation at one or more nucleotide positions selected from the group consisting of positions 20-33 of SEQ ID NO: 136; (iii) the variant of SEQ ID NO: 145 comprises a variation at one or more nucleotide positions selected from the group consisting of positions 50-1461 of SEQ ID NO: 145; (iv) the variant of SEQ ID NO: 146 comprises a variation at one or more nucleotide positions selected from the group consisting of positions 60-1441 of SEQ ID NO: 146; or (v) the variant of SEQ ID NO: 147 comprises a variation at one or more nucleotide positions selected from the group consisting of positions 60-897 of SEQ ID NO: 147.

In some embodiments, the virus is Respiratory Syncytial Virus (RSV), wherein the L region comprises a nucleotide sequence set forth as any one of SEQ ID NOs: 158, 163, 165, 166, or 419; or a variant of any one of SEQ ID NOs: 158, 163, 165, 166, or 419, and wherein the R region comprises the nucleotide sequence set forth as any one of SEQ ID NOs: 169, 170, 176, 177, or 420; or a variant of any one of SEQ ID NOS: 169, 170, 176, 177, or 420. In some embodiments, (i) the variant of SEQ ID NO: 158 comprises a variation at one or more nucleotide positions selected from the group consisting of positions 15-207 of SEQ ID NO: 158; (ii) the variant of SEQ ID NO: 163 comprises a variation at one or more nucleotide positions selected from the group consisting of positions 18-210 of SEQ ID NO: 163; (iii) the variant of SEQ ID NO: 165 comprises a variation at one or more nucleotide positions selected from the group consisting of positions 15-147 of SEQ ID NO: 165; (iv) the variant of SEQ ID NO: 166 comprises a variation at one or more nucleotide positions selected from the group consisting of positions 15-32 of SEQ ID NO: 166; or (v) the variant of SEQ ID NO: 419 comprises a variation at one or more nucleotide positions selected from the group consisting of positions 18-35 of SEQ ID NO: 419. In some embodiments, (i) the variant of SEQ ID NO: 169 comprises a variation at one or more nucleotide positions selected from the group consisting of positions 15-78 of SEQ ID NO: 169; (ii) the variant of SEQ ID NO: 170 comprises a variation at one or more nucleotide positions selected from the group consisting of positions 15-80 of SEQ ID NO: 170; (iii) the variant of SEQ ID NO: 176 comprises a variation at one or more nucleotide positions selected from the group consisting of positions 15-36 of SEQ ID NO: 176; (iv) the variant of SEQ ID NO: 177 comprises a variation at one or more nucleotide positions selected from the group consisting of positions 15-33 of SEQ ID NO: 177; or (v) the variant of SEQ ID NO: 420 comprises a variation at one or more nucleotide positions selected from the group consisting of positions 15-35 of SEQ ID NO: 420.

In some embodiments, the virus is a parainfluenzavirus, wherein the L region comprises a nucleotide sequence set forth as any one of SEQ ID NOs: 181, 182, or 183; or a variant of any one of SEQ ID NOs: 181, 182, or 183, and wherein the R region comprises the nucleotide sequence set forth as SEQ ID NO: 184; or a variant of SEQ ID NO: 184. In some embodiments, (i) the variant of SEQ ID NO: 181 comprises a variation at one or more nucleotide positions selected from the group consisting of positions 17-136 of SEQ ID NO: 181; (ii) the variant of SEQ ID NO: 182 comprises a variation at one or more nucleotide positions selected from the group consisting of positions 17-142 of SEQ ID NO: 182; or (iii) the variant of SEQ ID NO: 183 comprises a variation at one or more nucleotide positions selected from the group consisting of positions 17-136 of SEQ ID NO: 183. In some embodiments, the variant of SEQ ID NO: 184 comprises a variation at one or more nucleotide positions selected from the group consisting of positions 17-98 of SEQ ID NO: 184. In some embodiments, the virus is a parainfluenzavirus, wherein the L region comprises a nucleotide sequence set forth as any one of SEQ ID NOs: 187, 188, or 189; or a variant of any one of SEQ ID NOs: 187, 188, or 189, and wherein the R region comprises the nucleotide sequence set forth as SEQ ID NO: 190; or a variant of SEQ ID NO: 190. In some embodiments, (i) the variant of SEQ ID NO: 187 comprises a variation at one or more nucleotide positions selected from the group consisting of positions 21-95 of SEQ ID NO: 187; (ii) the variant of SEQ ID NO: 188 comprises a variation at one or more nucleotide positions selected from the group consisting of positions 21-101 of SEQ ID NO: 188; or (iii) the variant of SEQ ID NO: 189 comprises a variation at one or more nucleotide positions selected from the group consisting of positions 21-95 of SEQ ID NO: 189. In some embodiments, the variant of SEQ ID NO: 190 comprises a variation at one or more nucleotide positions selected from the group consisting of positions 21-93 of SEQ ID NO: 190.

In some embodiments, the virus is a metapneumovirus, wherein the L region comprises a nucleotide sequence set forth as any one of SEQ ID NOs: 196, 197, or 199; or a variant of any one of SEQ ID NOs: 196, 197, or 199, and wherein the R region comprises the nucleotide sequence set forth as SEQ ID NO: 201; or a variant of SEQ ID NO: 201. In some embodiments, (i) the variant of SEQ ID NO: 196 comprises a variation at one or more nucleotide positions selected from the group consisting of positions 21-224 of SEQ ID NO: 196; (ii) the variant of SEQ ID NO: 197 comprises a variation at one or more nucleotide positions selected from the group consisting of positions 21-230 of SEQ ID NO: 197; or (iii) the variant of SEQ ID NO: 199 comprises a variation at one or more nucleotide positions selected from the group consisting of positions 21-140 of SEQ ID NO: 199. In some embodiments, the variant of SEQ ID NO: 201 comprises a variation at one or more nucleotide positions selected from the group consisting of positions 17-32 of SEQ ID NO: 201. In some embodiments, the virus is a metapneumovirus, wherein the L region comprises a nucleotide sequence set forth as SEQ ID NO: 195; or a variant of SEQ ID NO: 195, and wherein the R region comprises the nucleotide sequence set forth as SEQ ID NO: 200; or a variant of SEQ ID NO: 200. In some embodiments, the variant of SEQ ID NO: 195 comprises a variation at one or more nucleotide positions selected from the group consisting of positions 21-224 of SEQ ID NO: 195. In some embodiments, the variant of SEQ ID NO: 200 comprises a variation at one or more nucleotide positions selected from the group consisting of positions 17-32 of SEQ ID NO: 200.

In some embodiments, the virus is a henipavirus, wherein the L region comprises a nucleotide sequence set forth as SEQ ID NO: 204; or a variant of SEQ ID NO: 204, and wherein the R region comprises the nucleotide sequence set forth as SEQ ID NO: 206; or a variant of SEQ ID NO: 206. In some embodiments, the variant of SEQ ID NO: 204 comprises a variation at one or more nucleotide positions selected from the group consisting of positions 17-77 of SEQ ID NO: 204. In some embodiments, the variant of SEQ ID NO: 206 comprises a variation at one or more nucleotide positions selected from the group consisting of positions 17-91 of SEQ ID NO: 206. In some embodiments, the virus is a henipavirus, wherein the L region comprises a nucleotide sequence set forth as SEQ ID NO: 209 or 210; or a variant of SEQ ID NO: 209 or 210, and wherein the R region comprises the nucleotide sequence set forth as SEQ ID NO: 211; or a variant of SEQ ID NO: 211. In some embodiments, (i) the variant of SEQ ID NO: 209 comprises a variation at one or more nucleotide positions selected from the group consisting of positions 17-77 of SEQ ID NO: 209; or (ii) the variant of SEQ ID NO: 210 comprises a variation at one or more nucleotide positions selected from the group consisting of positions 17-83 of SEQ ID NO: 210. In some embodiments, the variant of SEQ ID NO: 211 comprises a variation at one or more nucleotide positions selected from the group consisting of positions 17-91 of SEQ ID NO: 211.

In some embodiments, the virus is a hepadnavirus, wherein the L region comprises a nucleotide sequence set forth as SEQ ID NO: 222 or 223; or a variant of SEQ ID NO: 222 or 223 and wherein the R region comprises the nucleotide sequence set forth as SEQ ID NO: 225; or a variant of SEQ ID NO: 225. In some embodiments, (i) the variant of SEQ ID NO: 222 comprises a variation at one or more nucleotide positions selected from the group consisting of positions 101-639 of SEQ ID NO: 222. or (ii) the variant of SEQ ID NO: 223 comprises a variation at one or more nucleotide positions selected from the group consisting of positions 101-186 of SEQ ID NO: 223. In some embodiments, the variant of SEQ ID NO: 225 comprises a variation at one or more nucleotide positions selected from the group consisting of positions 101-1023 of SEQ ID NO: 225.

In some embodiments, the virus is a filovirus, wherein the L region comprises a nucleotide sequence set forth as any one of SEQ ID NOs: 227, 228, 229, or 230; or a variant of any one of SEQ ID NOs: 227, 228, 229, or 230, and wherein the R region comprises the nucleotide sequence set forth as SEQ ID NO: 231; or a variant of SEQ ID NO: 231. In some embodiments, (i) the variant of SEQ ID NO: 227 comprises a variation at one or more nucleotide positions selected from the group consisting of positions 20-710 of SEQ ID NO: 227; (ii) the variant of SEQ ID NO: 228 comprises a variation at one or more nucleotide positions selected from the group consisting of positions 23-713 of SEQ ID NO: 228; (iii) the variant of SEQ ID NO: 229 comprises a variation at one or more nucleotide positions selected from the group consisting of positions 17-707 of SEQ ID NO: 229; or (iv) the variant of SEQ ID NO: 230 comprises a variation at one or more nucleotide positions selected from the group consisting of positions 17-707 of SEQ ID NO: 230. In some embodiments, the variant of SEQ ID NO: 231 comprises a variation at one or more nucleotide positions selected from the group consisting of positions 15-449 of SEQ ID NO: 231. In some embodiments, the virus is a filovirus, wherein the L region comprises a nucleotide sequence set forth as any one of SEQ ID NOs: 232, 233, 234, or 235; or a variant of any one of SEQ ID NOs: 232, 233, 234, or 235, and wherein the R region comprises the nucleotide sequence set forth as SEQ ID NO: 236; or a variant of SEQ ID NO: 236. In some embodiments, (i) the variant of SEQ ID NO: 232 comprises a variation at one or more nucleotide positions selected from the group consisting of positions 20-678 of SEQ ID NO: 232; (ii) the variant of SEQ ID NO: 233 comprises a variation at one or more nucleotide positions selected from the group consisting of positions 23-681 of SEQ ID NO: 233; (iii) the variant of SEQ ID NO: 234 comprises a variation at one or more nucleotide positions selected from the group consisting of positions 23-678 of SEQ ID NO: 234; or (iv) the variant of SEQ ID NO: 235 comprises a variation at one or more nucleotide positions selected from the group consisting of positions 23-678 of SEQ ID NO: 235. In some embodiments, the variant of SEQ ID NO: 236 comprises a variation at one or more nucleotide positions selected from the group consisting of positions 15-437 of SEQ ID NO: 236.

In some embodiments, the virus is a filovirus, wherein the L region comprises a nucleotide sequence set forth as any one of SEQ ID NOs: 237, 238, or 239; or a variant of any one of SEQ ID NOs: 237, 238, or 239; and wherein the R region comprises the nucleotide sequence set forth as SEQ ID NO: 240; or a variant of SEQ ID NO: 240. In some embodiments, (i) the variant of SEQ ID NO: 237 comprises a variation at one or more nucleotide positions selected from the group consisting of positions 20-605 of SEQ ID NO: 237; (ii) the variant of SEQ ID NO: 238 comprises a variation at one or more nucleotide positions selected from the group consisting of positions 21-606 of SEQ ID NO: 238; or (iii) the variant of SEQ ID NO: 239 comprises a variation at one or more nucleotide positions selected from the group consisting of positions 20-605 of SEQ ID NO: 239. In some embodiments, the variant of SEQ ID NO: 240 comprises a variation at one or more nucleotide positions selected from the group consisting of positions 15-83 of SEQ ID NO: 240. In some embodiments, the virus is a filovirus, wherein the L region comprises a nucleotide sequence set forth as SEQ ID NO: 241; or a variant of SEQ ID NO: 241, and wherein the R region comprises the nucleotide sequence set forth as SEQ ID NO: 242; or a variant of any one of SEQ ID NO: 242. In some embodiments, the variant of SEQ ID NO: 241 comprises a variation at one or more nucleotide positions selected from the group consisting of positions 20-34 of SEQ ID NO: 241. In some embodiments, the variant of SEQ ID NO: 242 comprises a variation at one or more nucleotide positions selected from the group consisting of positions 100-593 of SEQ ID NO: 242. In some embodiments, the virus is a filovirus, wherein the L region comprises a nucleotide sequence set forth as SEQ ID NO: 243; or a variant of SEQ ID NO: 243, and wherein the R region comprises the nucleotide sequence set forth as SEQ ID NO: 244; or a variant of SEQ ID NO: 244. In some embodiments, the variant of SEQ ID NO: 243 comprises a variation at one or more nucleotide positions selected from the group consisting of positions 30-45 of SEQ ID NO: 243. In some embodiments, the variant of SEQ ID NO: 244 comprises a variation at one or more nucleotide positions selected from the group consisting of positions 100-677 of SEQ ID NO: 244. In some embodiments, the virus is a filovirus, wherein the L region comprises a nucleotide sequence set forth as any one of SEQ ID NOs: 245, 246, or 247; or a variant of any one of SEQ ID NOs: 245, 246, or 247, and wherein the R region comprises the nucleotide sequence set forth as SEQ ID NO: 248; or a variant of SEQ ID NO: 248. In some embodiments, (i) the variant of SEQ ID NO: 245 comprises a variation at one or more nucleotide positions selected from the group consisting of positions 29-171 of SEQ ID NO: 245; (ii) the variant of SEQ ID NO: 246 comprises a variation at one or more nucleotide positions selected from the group consisting of positions 30-171 of SEQ ID NO: 246; or (iii) the variant of SEQ ID NO: 247 comprises a variation at one or more nucleotide positions selected from the group consisting of positions 29-171 of SEQ ID NO: 247. In some embodiments, the variant of SEQ ID NO: 248 comprises a variation at one or more nucleotide positions selected from the group consisting of positions 20-91 of SEQ ID NO: 248.

In some embodiments, the virus is an alphavirus, wherein the L region comprises a nucleotide sequence set forth as SEQ ID NO: 249; or a variant of SEQ ID NO: 249, and wherein the R region comprises the nucleotide sequence set forth as SEQ ID NO: 250 or 251; or a variant of SEQ ID NO: 250 or 251. In some embodiments, the variant of SEQ ID NO: 249 comprises a variation at one or more nucleotide positions selected from the group consisting of positions 60-274 of SEQ ID NO: 249. In some embodiments, (i) the variant of SEQ ID NO: 250 comprises a variation at one or more nucleotide positions selected from the group consisting of positions 60-183 of SEQ ID NO: 250; or (ii) the variant of SEQ ID NO: 251 comprises a variation at one or more nucleotide positions selected from the group consisting of positions 60-375 of SEQ ID NO: 251. In some embodiments, the virus is an alphavirus, wherein the L region comprises a nucleotide sequence set forth as SEQ ID NO: 255; or a variant of SEQ ID NO: 255 and wherein the R region comprises the nucleotide sequence set forth as SEQ ID NO: 256 or 257; or a variant of SEQ ID NO: 256 or 257. In some embodiments, the variant of SEQ ID NO: 255 comprises a variation at one or more nucleotide positions selected from the group consisting of positions 60-35 of SEQ ID NO: 255. In some embodiments, (i) the variant of SEQ ID NO: 256 comprises a variation at one or more nucleotide positions selected from the group consisting of positions 600-273 of SEQ ID NO: 256; or (ii) the variant of SEQ ID NO: 257 comprises a variation at one or more nucleotide positions selected from the group consisting of positions 60-377 of SEQ ID NO: 257. In some embodiments, the virus is an alphavirus, wherein the L region comprises a nucleotide sequence set forth as SEQ ID NO: 261; or a variant of SEQ ID NO: 261, and wherein the R region comprises the nucleotide sequence set forth as SEQ ID NO: 262 or 263; or a variant of SEQ ID NO: 262 or 263. In some embodiments, the variant of SEQ ID NO: 261 comprises a variation at one or more nucleotide positions selected from the group consisting of positions 60-215 of SEQ ID NO: 261. In some embodiments, (i) the variant of SEQ ID NO: 262 comprises a variation at one or more nucleotide positions selected from the group consisting of positions 60-166 of SEQ ID NO: 262; or (ii) the variant of SEQ ID NO: 263 comprises a variation at one or more nucleotide positions selected from the group consisting of positions 60-379 of SEQ ID NO: 263.

In some embodiments, the virus is a sarbecovirus, wherein the L region comprises a nucleotide sequence set forth as any one of SEQ ID NOs: 68, 69, 70, 71, 72, 73, 74, 75, 76, or 77; or a variant of any one of SEQ ID NOW: 68, 69, 70, 71, 72, 73, 74, 75, 76, or 77, and wherein the R region comprises the nucleotide sequence set forth as SEQ ID NO: 130; or a variant of SEQ ID NO: 130. In some embodiments, (i) the variant of SEQ ID NO: 68 comprises a variation at one or more nucleotide positions selected from the group consisting of positions 39-789 or 1434-1501 of SEQ ID NO: 68; (ii) the variant of SEQ ID NO: 69 comprises a variation at one or more nucleotide positions selected from the group consisting of positions 39-789 or 1434-1501 of SEQ ID NO: 69; (iii) the variant of SEQ ID NO: 70 comprises a variation at one or more nucleotide positions selected from the group consisting of positions 39-789 or 1446-1513 of SEQ ID NO: 70; (iv) the variant of SEQ ID NO: 71 comprises a variation at one or more nucleotide positions selected from the group consisting of positions 39-789 or 1455-1522 of SEQ ID NO: 71; (v) the variant of SEQ ID NO: 72 comprises a variation at one or more nucleotide positions selected from the group consisting of positions 39-789 or 1462-1529 of SEQ ID NO: 72; (vi) the variant of SEQ ID NO: 73 comprises a variation at one or more nucleotide positions selected from the group consisting of positions 39-789 or 1469-1536 of SEQ ID NO: 73; (vii) the variant of SEQ ID NO: 74 comprises a variation at one or more nucleotide positions selected from the group consisting of positions 39-789 or 1485-1552 of SEQ ID NO: 74; (viii) the variant of SEQ ID NO: 75 comprises a variation at one or more nucleotide positions selected from the group consisting of positions 39-789 or 1686-1753 of SEQ ID NO: 75; (ix) the variant of SEQ ID NO: 76 comprises a variation at one or more nucleotide positions selected from the group consisting of positions 39-789 or 1704-1771 of SEQ ID NO: 76; or (x) the variant of SEQ ID NO: 77 comprises a variation at one or more nucleotide positions selected from the group consisting of positions 39-789 or 1720-1787 of SEQ ID NO: 77. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 78, 79, 80, 81, 82, 83, 85, 86, 87, or 88; or a variant of any one of SEQ ID NOs: 78, 79, 80, 81, 82, 83, 85, 86, 87, or 88, and wherein the R region comprises the nucleotide sequence set forth as SEQ ID NO: 130; or a variant of SEQ ID NO: 130. In some embodiments, (i) the variant of SEQ ID NO: 78 comprises a variation at one or more nucleotide positions selected from the group consisting of positions 39-789 or 1734-1801 of SEQ ID NO: 78; (ii) the variant of SEQ ID NO: 79 comprises a variation at one or more nucleotide positions selected from the group consisting of positions 39-789 or 1687-1754 of SEQ ID NO: 79; (iii) the variant of SEQ ID NO: 80 comprises a variation at one or more nucleotide positions selected from the group consisting of positions 39-789 or 1695-1762 of SEQ ID NO: 80; (iv) the variant of SEQ ID NO: 81 comprises a variation at one or more nucleotide positions selected from the group consisting of positions 39-789 or 1434-1501 of SEQ ID NO: 81; (v) the variant of SEQ ID NO: 82 comprises a variation at one or more nucleotide positions selected from the group consisting of positions 39-789 or 1443-1510 of SEQ ID NO: 82; (vi) the variant of SEQ ID NO: 83 comprises a variation at one or more nucleotide positions selected from the group consisting of positions 39-789 or 1459-1526 of SEQ ID NO: 83; (vii) the variant of SEQ ID NO: 85 comprises a variation at one or more nucleotide positions selected from the group consisting of positions 39-789 or 1434-1501 of SEQ ID NO: 85; (viii) the variant of SEQ ID NO: 86 comprises a variation at one or more nucleotide positions selected from the group consisting of positions 39-789 or 1434-1501 of SEQ ID NO: 86; (ix) the variant of SEQ ID NO: 87 comprises a variation at one or more nucleotide positions selected from the group consisting of positions 39-789 or 1435-1502 of SEQ ID NO: 87; or (x) the variant of SEQ ID NO: 88 comprises a variation at one or more nucleotide positions selected from the group consisting of positions 39-789 or 1463-1530 of SEQ ID NO: 88. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 89, 90, 91, 92, 96, 104, 105, 106, 107, or 108; or a variant of any one of SEQ ID NOs: 89, 90, 91, 92, 96, 104, 105, 106, 107, or 108, and wherein the R region comprises the nucleotide sequence set forth as SEQ ID NO: 130; or a variant of SEQ ID NO: 130. In some embodiments, (i) the variant of SEQ ID NO: 89 comprises a variation at one or more nucleotide positions selected from the group consisting of positions 39-789 or 1466-1533 of SEQ ID NO: 89; (ii) the variant of SEQ ID NO: 90 comprises a variation at one or more nucleotide positions selected from the group consisting of positions 39-789 or 1425-1492 of SEQ ID NO: 90; (iii) the variant of SEQ ID NO: 91 comprises a variation at one or more nucleotide positions selected from the group consisting of positions 39-789 or 1425-1492 of SEQ ID NO: 91; (iv) the variant of SEQ ID NO: 92 comprises a variation at one or more nucleotide positions selected from the group consisting of positions 39-789 or 1425-1492 of SEQ ID NO: 92; (v) the variant of SEQ ID NO: 96 comprises a variation at one or more nucleotide positions selected from the group consisting of positions 39-769 or 1471-1471 of SEQ ID NO: 96; (vi) the variant of SEQ ID NO: 104 comprises a variation at one or more nucleotide positions selected from the group consisting of positions 39-789 or 1446-1513 of SEQ ID NO: 104; (vii) the variant of SEQ ID NO: 105 comprises a variation at one or more nucleotide positions selected from the group consisting of positions 39-789 or 1455-1522 of SEQ ID NO: 105; (viii) the variant of SEQ ID NO: 106 comprises a variation at one or more nucleotide positions selected from the group consisting of positions 39-789 or 1462-1529 of SEQ ID NO: 106; (ix) the variant of SEQ ID NO: 107 comprises a variation at one or more nucleotide positions selected from the group consisting of positions 39-789 or 1469-1536 of SEQ ID NO: 107; or (x) the variant of SEQ ID NO: 108 comprises a variation at one or more nucleotide positions selected from the group consisting of positions 89-839 or 1485-1552 of SEQ ID NO: 108. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 109, 110, 111, 112, 113, 114, 115, 116, 117, or 118; or a variant of any one of SEQ ID NOs: 109, 110, 111, 112, 113, 114, 115, 116, 117, or 118, and wherein the R region comprises the nucleotide sequence set forth as SEQ ID NO: 130; or a variant of SEQ ID NO: 130. In some embodiments, (i) the variant of SEQ ID NO: 109 comprises a variation at one or more nucleotide positions selected from the group consisting of positions 40-789 or 1686-1753 of SEQ ID NO: 109; (ii) the variant of SEQ ID NO: 110 comprises a variation at one or more nucleotide positions selected from the group consisting of positions 40-789 or 1704-1771 of SEQ ID NO: 110; (iii) the variant of SEQ ID NO: 111 comprises a variation at one or more nucleotide positions selected from the group consisting of positions 40-789 or 1720-1787 of SEQ ID NO: 111; (iv) the variant of SEQ ID NO: 112 comprises a variation at one or more nucleotide positions selected from the group consisting of positions 40-789 or 1734-1801 of SEQ ID NO: 112; (v) the variant of SEQ ID NO: 113 comprises a variation at one or more nucleotide positions selected from the group consisting of positions 40-789 or 1687-1754 of SEQ ID NO: 113; (vi) the variant of SEQ ID NO: 114 comprises a variation at one or more nucleotide positions selected from the group consisting of positions 40-789 or 1695-1762 of SEQ ID NO: 114; (vii) the variant of SEQ ID NO: 115 comprises a variation at one or more nucleotide positions selected from the group consisting of positions 40-789 or 1434-1501 of SEQ ID NO: 115; (viii) the variant of SEQ ID NO: 116 comprises a variation at one or more nucleotide positions selected from the group consisting of positions 40-789 or 1434-1501 of SEQ ID NO: 116; (ix) the variant of SEQ ID NO: 117 comprises a variation at one or more nucleotide positions selected from the group consisting of positions 40-789 or 1434-1501 of SEQ ID NO: 117; or (xl) the variant of SEQ ID NO: 118 comprises a variation at one or more nucleotide positions selected from the group consisting of positions 40-789 or 1434-1501 of SEQ ID NO: 118. In some embodiments, the variant of SEQ ID NO: 130 comprises a variation at one or more nucleotide positions selected from the group consisting of positions 20-320 of SEQ ID NO: 130.

In some embodiments, the virus is a sarbecovirus, wherein the L region comprises a nucleotide sequence set forth as any one of SEQ ID NOs: 119, 120, 122, 123, 124, 125, 126, or 127; or a variant of any one of SEQ ID NOs: 119, 120, 122, 123, 124, 125, 126, or 127; and wherein the R region comprises the nucleotide sequence set forth as SEQ ID NO: 130; or a variant of SEQ ID NO: 130. In some embodiments, (i) the variant of SEQ ID NO: 119 comprises a variation at one or more nucleotide positions selected from the group consisting of positions 40-789 or 1443-1510 of SEQ ID NO: 119; (ii) the variant of SEQ ID NO: 120 comprises a variation at one or more nucleotide positions selected from the group consisting of positions 40-789 or 1459-1526 of SEQ ID NO: 120; (iii) the variant of SEQ ID NO: 122 comprises a variation at one or more nucleotide positions selected from the group consisting of positions 40-789 or 1434-1501 of SEQ ID NO: 122; (iv) the variant of SEQ ID NO: 123 comprises a variation at one or more nucleotide positions selected from the group consisting of positions 40-789 or 1434-1501 of SEQ ID NO: 123; (v) the variant of SEQ ID NO: 124 comprises a variation at one or more nucleotide positions selected from the group consisting of positions 40-789 or 1434-1501 of SEQ ID NO: 124; (vi) the variant of SEQ ID NO: 125 comprises a variation at one or more nucleotide positions selected from the group consisting of positions 40-789 or 1463-1530 of SEQ ID NO: 125; (vii) the variant of SEQ ID NO: 126 comprises a variation at one or more nucleotide positions selected from the group consisting of positions 40-789 or 1466-1533 of SEQ ID NO: 126; (viii) the variant of SEQ ID NO: 127 comprises a variation at one or more nucleotide positions selected from the group consisting of positions 40-789 or 1425-1492 of SEQ ID NO: 127. In some embodiments, the variant of SEQ ID NO: 130 comprises a variation at one or more nucleotide positions selected from the group consisting of positions 20-320 of SEQ ID NO: 130.

In some embodiments, the virus is a Respiratory Syncytial Virus (RSV), wherein the L region comprises a nucleotide sequence set forth as any one of SEQ ID NOs: 148, 149, 150, 151, or 152; or a variant of any one of SEQ ID NOs: 148, 149, 150, 151, or 152, and wherein the R region comprises the nucleotide sequence set forth as SEQ ID NO: 154 or 155. In some embodiments, (i) the variant of SEQ ID NO: 148 comprises a variation at one or more nucleotide positions selected from the group consisting of positions 15-78 of SEQ ID NO: 148; (ii) the variant of SEQ ID NO: 149 comprises a variation at one or more nucleotide positions selected from the group consisting of positions 15-33 of SEQ ID NO: 149; (iii) the variant of SEQ ID NO: 150 comprises a variation at one or more nucleotide positions selected from the group consisting of positions 15-35 of SEQ ID NO: 150; (iv) the variant of SEQ ID NO: 151 comprises a variation at one or more nucleotide positions selected from the group consisting of positions 18-36 of SEQ ID NO: 151; or (v) the variant of SEQ ID NO: 152 comprises a variation at one or more nucleotide positions selected from the group consisting of positions 15-38 of SEQ ID NO: 152. In some embodiments, (i) the variant of SEQ ID NO: 154 comprises a variation at one or more nucleotide positions selected from the group consisting of positions 15-207 of SEQ ID NO: 154; or (ii) the variant of SEQ ID NO: 155 comprises a variation at one or more nucleotide positions selected from the group consisting of positions 15-32 of SEQ ID NO: 155.

In some embodiments, the virus is a parainfluenzavirus, wherein the L region comprises a nucleotide sequence set forth as SEQ ID NO: 180; or a variant of SEQ ID NO: 180, and wherein the R region comprises the nucleotide sequence set forth as SEQ ID NO: 179; or a variant of SEQ ID NO: 179. In some embodiments, the variant of SEQ ID NO: 180 comprises a variation at one or more nucleotide positions selected from the group consisting of positions 17-136 of SEQ ID NO: 180. In some embodiments, the variant of SEQ ID NO: 179 comprises a variation at one or more nucleotide positions selected from the group consisting of positions 17-98 of SEQ ID NO: 179. In some embodiments, the virus is a parainfluenzavirus, wherein the L region comprises a nucleotide sequence set forth as SEQ ID NO: 186; or a variant of SEQ ID NO: 186, and wherein the R region comprises the nucleotide sequence set forth as SEQ ID NO: 185; or a variant of SEQ ID NO: 185. In some embodiments, the variant of SEQ ID NO: 186 comprises a variation at one or more nucleotide positions selected from the group consisting of positions 21-95 of SEQ ID NO: 186. In some embodiments, the variant of SEQ ID NO: 185 comprises a variation at one or more nucleotide positions selected from the group consisting of positions 21-93 of SEQ ID NO: 185.

In some embodiments, the virus is a metapneumovirus, wherein the L region comprises a nucleotide sequence set forth as SEQ ID NO: 194; or a variant of SEQ ID NO: 194, and wherein the R region comprises the nucleotide sequence set forth as SEQ ID NO: 192; or a variant of any one of SEQ ID NO: 192. In some embodiments, the variant of SEQ ID NO: 194 comprises a variation at one or more nucleotide positions selected from the group consisting of positions 17-220 of SEQ ID NO: 194. In some embodiments, the variant of SEQ ID NO: 192 comprises a variation at one or more nucleotide positions selected from the group consisting of positions 17-32 of SEQ ID NO: 192. In some embodiments, the virus is a metapneumovirus, wherein the L region comprises a nucleotide sequence set forth as SEQ ID NO: 193; or a variant of SEQ ID NO: 193, and wherein the R region comprises the nucleotide sequence set forth as SEQ ID NO: 191; or a variant of SEQ ID NO: 191. In some embodiments, the variant of SEQ ID NO: 193 comprises a variation at one or more nucleotide positions selected from the group consisting of positions 17-220 of SEQ ID NO: 193. In some embodiments, the variant of SEQ ID NO: 191 comprises a variation at one or more nucleotide positions selected from the group consisting of positions 17-32 of SEQ ID NO: 191.

In some embodiments, the virus is a henipavirus, wherein the L region comprises a nucleotide sequence set forth as SEQ ID NO: 203; or a variant of SEQ ID NO: 203, and wherein the R region comprises the nucleotide sequence set forth as SEQ ID NO: 202; or a variant of SEQ ID NO: 202. In some embodiments, the variant of SEQ ID NO: 203 comprises a variation at one or more nucleotide positions selected from the group consisting of positions 17-77 of SEQ ID NO: 203. In some embodiments, the variant of SEQ ID NO: 202 comprises a variation at one or more nucleotide positions selected from the group consisting of positions 17-91 of SEQ ID NO: 202. In some embodiments, the virus is a henipavirus, wherein the L region comprises a nucleotide sequence set forth as SEQ ID NO: 207; or a variant of SEQ ID NO: 207, and wherein the R region comprises the nucleotide sequence set forth as SEQ ID NO: 208; or a variant of SEQ ID NO: 208. In some embodiments, the variant of SEQ ID NO: 207 comprises a variation at one or more nucleotide positions selected from the group consisting of positions 17-77 of SEQ ID NO: 207. In some embodiments, the variant of SEQ ID NO: 208 comprises a variation at one or more nucleotide positions selected from the group consisting of positions 17-91 of SEQ ID NO: 208.

In some embodiments, the virus is a hepadnavirus, wherein the L region comprises a nucleotide sequence set forth as any one of SEQ ID NOs: 212, 213, 214, 215, or 216; or a variant of any one of SEQ ID NOs: 212, 213, 214, 215, or 216, and wherein the R region comprises the nucleotide sequence set forth as any one of SEQ ID NOs: 217, 218, 219, or 220; or a variant of any one of SEQ ID NOs: 217, 218, 219, or 220. In some embodiments, (i) the variant of SEQ ID NO: 212 comprises a variation at one or more nucleotide positions selected from the group consisting of positions 101-1326 of SEQ ID NO: 212; (ii) the variant of SEQ ID NO: 213 comprises a variation at one or more nucleotide positions selected from the group consisting of positions 101-1291 of SEQ ID NO: 213; (iii) the variant of SEQ ID NO: 214 comprises a variation at one or more nucleotide positions selected from the group consisting of positions 101-1325 of SEQ ID NO: 214; (iv) the variant of SEQ ID NO: 215 comprises a variation at one or more nucleotide positions selected from the group consisting of positions 101-15 of SEQ ID NO: 215; or (v) the variant of SEQ ID NO: 216 comprises a variation at one or more nucleotide positions selected from the group consisting of positions 101-211 of SEQ ID NO: 216. In some embodiments, (i) the variant of SEQ ID NO: 217 comprises a variation at one or more nucleotide positions selected from the group consisting of positions 101-754 of SEQ ID NO: 217; (ii) the variant of SEQ ID NO: 218 comprises a variation at one or more nucleotide positions selected from the group consisting of positions 101-790 of SEQ ID NO: 218; (iii) the variant of SEQ ID NO: 219 comprises a variation at one or more nucleotide positions selected from the group consisting of positions 101-892 of SEQ ID NO: 219; or (iv) the variant of SEQ ID NO: 220 comprises a variation at one or more nucleotide positions selected from the group consisting of positions 101-2309 of SEQ ID NO: 220.

In some embodiments, the virus is an alphavirus, wherein the L region comprises a nucleotide sequence set forth as any one of SEQ ID NO: 252 or 253; or a variant of any one of SEQ ID NO: 252 or 253, and wherein the R region comprises the nucleotide sequence set forth as any one of SEQ ID NO: 254; or a variant of any one of SEQ ID NO: 254. In some embodiments, (i) the variant of SEQ ID NO: 252 comprises a variation at one or more nucleotide positions selected from the group consisting of positions 100-223 of SEQ ID NO: 252; or (ii) the variant of SEQ ID NO: 253 comprises a variation at one or more nucleotide positions selected from the group consisting of positions 100-415 of SEQ ID NO: 253. In some embodiments, (i) the variant of SEQ ID NO: 254 comprises a variation at one or more nucleotide positions selected from the group consisting of positions 100-321 of SEQ ID NO: 254. In some embodiments, the virus is an alphavirus, wherein the L region comprises a nucleotide sequence set forth as any one of SEQ ID NO: 258 or 259; or a variant of any one of SEQ ID NO: 258 or 259, and wherein the R region comprises the nucleotide sequence set forth as any one of SEQ ID NO: 260; or a variant of any one of SEQ ID NO: 260. In some embodiments, (i) the variant of SEQ ID NO: 258 comprises a variation at one or more nucleotide positions selected from the group consisting of positions 100-323 of SEQ ID NO: 258; or (ii) the variant of SEQ ID NO: 259 comprises a variation at one or more nucleotide positions selected from the group consisting of positions 100-427 of SEQ ID NO: 259. In some embodiments, the variant of SEQ ID NO: 260 comprises a variation at one or more nucleotide positions selected from the group consisting of positions 60-84 of SEQ ID NO: 260.

In some embodiments, the virus is an alphavirus, wherein the L region comprises a nucleotide sequence set forth as any one of SEQ ID NO: 264 or 265; or a variant of any one of SEQ ID NO: 264 or 265, and wherein the R region comprises the nucleotide sequence set forth as any one of SEQ ID NO: 266; or a variant of any one of SEQ ID NO: 266. In some embodiments, (i) the variant of SEQ ID NO: 264 comprises a variation at one or more nucleotide positions selected from the group consisting of positions 100-216 of SEQ ID NO: 264; or (ii) the variant of SEQ ID NO: 265 comprises a variation at one or more nucleotide positions selected from the group consisting of positions 100-429 of SEQ ID NO: 265. In some embodiments, the variant of SEQ ID NO: 266 comprises a variation at one or more nucleotide positions selected from the group consisting of positions 60-262 of SEQ ID NO: 266.

Aspects of the present disclosure provide isolated DNA polynucleotides encoding any of the isolated RNA polynucleotides described herein.

Other aspects provide a cell or cell line comprising any of the isolated DNA polynucleotides described herein.

Other aspects provide vectors comprising any of the isolated RNA polynucleotides or isolated DNA polynucleotides described herein. In some embodiments, the vector is a viral vector or an expression vector. In some embodiments, the viral vector is selected from the group consisting of adenovirus vector, adeno-associated virus vector, poxvirus vector, retrovirus vector, lentivirus vector, herpesvirus vector, alphavirus vector, and baculovirus vector.

Other aspects of the present disclosure provide an RNA-protein complex comprising any of the isolated RNA polynucleotides described herein and an RNA-binding protein; wherein the isolated RNA polynucleotide of the RNA-protein complex has increased stability as compared to the isolated RNA polypeptide without the RNA binding protein. In some embodiments, the RNA-binding protein is a viral nucleocapsid protein (N) or viral capsid protein. In some embodiments, the RNA-binding protein is a viral nucleocapsid protein (N) or a viral capsid protein of the virus. In some embodiments, the viral nucleocapsid protein or a viral capsid protein from an influenza virus, sarbecovirus, pneumovirus, paramyxovirus, henipavirus, or hepadnavirus.

Other aspects of the present disclosure provide compositions comprising any of the isolated RNA polynucleotides, isolated DNA polynucleotides, cell or cell line, vector, or RNA-protein complexes described herein. In some embodiments, the composition further comprises a pharmaceutically acceptable carrier.

Yet other aspects provide nanoparticles comprising any of the isolated RNA polynucleotides, isolated DNA polynucleotides, or the RNA-protein complexes described herein.

Aspects of the present disclosure provide methods comprising administering to a subject in need thereof a therapeutically effective amount of any of the isolated RNA polynucleotides, the isolated DNA polynucleotides, the cells or cell lines, the vectors, the RNA-protein complexes, the compositions, or nanoparticles described herein. In some embodiments, the method further comprises administering to a subject in need thereof a therapeutically effective amount of any of a second isolated RNA polynucleotide, a second isolated DNA polynucleotide, a second cell or cell line, a second vector, a second RNA-protein complex, a second composition, or a second nanoparticle, wherein the second entity is different from the first entity (e.g., the second isolated RNA polynucleotide is different than the first isolated RNA polynucleotide administered to the subject). In some embodiments, the subject is a human, cow, pig, sheep, horse, deer, rumenants, rodent, fish, or fowl.

In some embodiments, the subject has a disease or disorder resulting from a viral infection. In some embodiments, the subject has an infection with a virus.

In some embodiments, the administration is by intratracheal or inhalation, intranasal, oral, rectal, vaginal, transmucosal, or intestinal administration; parenteral delivery, including intradermal, transdermal (topical), intramuscular, subcutaneous, intramedullary injections, as well as intrathecal, direct intraventricular, intravenous, or intraperitoneal administration.

Aspects of the present disclosure provide methods comprising contacting a cell with any of the isolated RNA polynucleotides, the isolated DNA polynucleotides, the cells or cell lines, the vectors, the RNA-protein complexes, the compositions, or nanoparticles described herein. In some embodiments, the contacting is in vitro or ex vivo.

Aspects of the present disclosure provide methods comprising administering to a subject in need thereof (i) a therapeutically effective amount of any of the isolated RNA polynucleotides, the isolated DNA polynucleotides, the cells or cell lines, the vectors, the RNA-protein complexes, the compositions, or nanoparticles described herein; and (ii) a second polynucleotide encoding a polymerase capable of interacting with and initiating the transcription or translation of the therapeutic polypeptide or polypeptide. In some embodiments, the method further comprises administering to the subject (iii) one or more accessory proteins associated with polymerase activity. In some embodiments, the accessory protein is a nucleocapsid protein. In some embodiments, the polymerase and/or accessory proteins are administered in the form of one or more nucleic acid encoding the polymerase and/or accessory proteins. In some embodiments, (i) and (ii) are administered sequentially or simultaneously. In some embodiments, (i) and (ii) are present on the same polynucleotide. In some embodiments, (i) and (ii) are present on separate polynucleotides.

In some embodiments, the method further comprises administering to a subject in need thereof a therapeutically effective amount of a any of the isolated RNA polynucleotides, the isolated DNA polynucleotides, the cells or cell lines, the vectors, the RNA-protein complexes, the compositions, or nanoparticles described herein. In some embodiments, the subject is a human, cow, pig, sheep, horse, deer, rumenants, rodent, fish, or fowl.

Other aspects of the present disclosure provide method comprising (a) providing a DNA vector encoding any of the isolated RNA polynucleotide described herein; (b) linearizing the DNA vector to produce a linear DNA vector; and (c) contacting the linear DNA vector with a RNA polymerase, thereby producing the isolated RNA polynucleotide. In some embodiments, the method further comprises (d) subjecting the isolated RNA polynucleotide of (c) to one or more purification steps. In some embodiments, the one or more purification steps of (d) are selected from contacting the isolated RNA polynucleotide with DNAse under conditions suitable for the digestion of the DNA vector; and tangential flow filtration. In some embodiments, the DNA vector comprises a promoter capable of directing activity of the RNA polymerase and/or a restriction endonuclease recognition site. In some embodiments, the RNA polymerase is a T7 RNA polymerase and the promoter is a T7 promoter. In some embodiments, linearizing the DNA vector comprises contacting the DNA vector with a restriction endonuclease that recognizes the restriction endonuclease recognition site. In some embodiments, the contacting of (c) is performed at about 50° C. In some embodiments, the contacting of (c) is performed in the presence of one or more additional factors selected from the group consisting of ribonucleotide triphosphates, modified nucleotide triphosphates, a cap analog, inorganic pyrophosphatase, and a RNAse inhibitor. In some embodiments, the method further comprises formulating the isolated RNA polynucleotide into a nanoparticle.

Other aspects of the present disclosure provide methods of generating a transgenic animal or plant comprising inserting any of the isolated RNA polynucleotides, the isolated DNA polynucleotides, the cells or cell lines, the vectors, the RNA-protein complexes, or the compositions, or the nanoparticles into an animal or plant, thereby generating a transgenic animal or plant. In some embodiments, the coding sequence of the encodes an antiviral polypeptide. In some embodiments, the transgenic animal or plant has increased resistance to viral infection. In some embodiments, the transgenic animal or plant is an avian, pig, fish, cow, horse, camel, dog, cat, mouse, rat, cotton rat, hamster, ferret, primate, or other commercially valuable animal or plant species.

In one aspect the present disclosure provides an isolated ribonucleic acid (RNA) polynucleotide including (a) a coding region which encodes a therapeutic polypeptide of interest and (b) a template region for binding a target-specific translation activator. The isolated polynucleotide interacts with the translation activator causing transcription and ultimately translation of the therapeutic polypeptide of interest in increased amounts in a cell containing said RNA polynucleotide. In some embodiments, the translation activator is a polymerase. In some embodiments, the polymerase is an RNA-dependent RNA polymerase or RNA-Dependent DNA Polymerase. In some embodiments, the template region of the isolated RNA or the translation activator is not derived from an alphavirus genome. In some embodiments, the RNA is a single-stranded RNA polynucleotide. In some embodiments, the coding region for the polypeptide of interest is on the sense or antisense strand.

The isolated ribonucleic acid (RNA) polynucleotide in embodiments can incorporate a nucleoside that is not adenosine, cytidine, guanosine, or uridine. In some embodiments, the isolated ribonucleic acid (RNA) polynucleotide in the 5′ terminus is capped. In some embodiments, it is uncapped. In some embodiments, the isolated ribonucleic acid (RNA) polynucleotide is 5′-monophosphorylated or 5′-nonphosphorylated.

The isolated ribonucleic acid (RNA) polynucleotides of the present disclosure can be in linear or can be in covalently-closed circular form.

In some embodiments, the isolated ribonucleic acid (RNA) polynucleotide has increased immunogenicity after it is contacted by a translation activator.

In some embodiments, isolated ribonucleic acid (RNA) polynucleotide has a coding region that codes for an interferon, an interferon stimulated gene, an antibody, a signaling molecule, a cytotoxic protein, a protein that causes cell death, an antineoplastic protein, an immunomodulatory protein, or a dominant negative protein. In some embodiments, the coding region codes for both a pro-inflammatory cytokine and an anti-inflammatory cytokine. In some embodiments, the coding region codes for an interleukin-1 receptor antagonist. In some embodiments, the coding region codes for an interleukin or a caspase. In some embodiments, the coding region codes for a protein with antiviral activity. In some embodiments, the coding region codes for a secreted protein, which may an antibody or an interferon including IFN-α, IFN-β, IFN-ε, IFN-κ, IFN-ω, IFN-γ, or IFN-λ, IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-β1, IFN-ε, IFN-κ, IFN-ω1, IFN-γ, IFN-λ1 (IL28A), IFN-λ2 (IL28B), IFN-λ3 (IL29), or IFN-λ4. In some embodiments, the interferon is IFN-α, IFN-β, IFN-λ1 (IL28A), IFN-λ2 (IL28B), or IFN-λ3 (IL29).

In some embodiments the target-specific translation activator comprises a viral RNA-Dependent RNA Polymerase. The viral RNA-dependent RNA polymerase or the RNA-Dependent DNA Polymerase in some embodiments is produced from a viral genome during viral infection. In some embodiments the target-specific translation activator is an Influenza A polymerase, Influenza B polymerase, respiratory syncytial virus (RSV) polymerase, coronavirus polymerase, sarbecovirus polymerase, metapneumovirus polymerase, parainfluenza virus polymerase, or henipavirus polymerase. In some embodiments, the polymerase is an NL63, OC43, 229E, HKU-1, SARS-COV-1, SARS-COV-2, or MERS-COV polymerase. In some embodiments, the target-specific translation activator comprises a hepadnavirus polymerase or a hepatitis B virus polymerase. The target-specific translation activator may comprise additional polypeptide(s) required for mRNA synthesis, for example a matrix protein or nucleoprotein.

In some embodiments, the isolated RNA includes a left flanking region (“L”) comprised of a cis-acting sequence; a central region (“C”) comprised of the coding region for the polypeptide of interest; and a right flanking region (“R”) comprised of a cis-acting sequence; wherein region L and R together allow for the target-specific translation activator to direct transcription of mRNA that is distinct from the isolated RNA that codes for the therapeutic polypeptide of interest. In some embodiments, region L is comprised of a sequence in Table 1 for which the flank is identified as “L” and the encryption is identified as “antisense”. In some embodiments, region C is comprised of an antisense protein coding sequence. In some embodiments, region R is comprised of a sequence in Table 1 for which the flank is identified as “R” and the encryption is identified as “antisense”. In some embodiments, region L is comprised of a sequence in Table 1 for which the flank is identified as “L” and the encryption is identified as “sense”, region C is comprised of a sense protein coding sequence, and region R is comprised of a sequence in Table 1 for which the flank is identified as “R” and the encryption is identified as “sense”.

In some embodiments, region L is comprised of a sequence L′ and region R is comprised of a sequence R′, where L′ is an L sequence from Table 2 and R′ is an R sequence from Table 2, and L′ and R′ share the same Encrypted RNA Scaffold. In some embodiments, the Encrypted RNA Scaffold is antisense and in some it is sense.

According to the present disclosure, region C can encode one or more than one polypeptide of interest. In some embodiments, the more than one polypeptides of interest are separated by ribosomal skipping sites. In some embodiments involving more than one polypeptide of interest, the isolated RNA has a structure of: a first central region (“C1”) comprised of a coding region for a polypeptide of interest; one or more additional coding regions, each having an internal flanking region (“I”) comprised of a cis-acting sequence, separating a preceding polypeptide of interest from a subsequent polypeptide of interest; and a subsequent region comprised of a coding region for a subsequent polypeptide of interest. In some embodiments, the encryption of the Encrypted RNA is antisense and the internal flanking region (“I”) is selected from paramyxovirus or pneumovirus gene start sequences. In some embodiments, region L is comprised of a sequence L′ and region R is comprised of a sequence R′, where L′ is an L sequence from Table 2 and R′ is an R sequence from Table 2, and L′ and R′ share the same Encrypted RNA Scaffold, and the target virus is RSV. In some embodiments, the internal flanking regions (“I”), are selected from Table 1 where the Target Virus is identified as “RSV” and the Flank is identified as “I1”.

The present disclosure also embraces an isolated DNA that encodes the isolated RNA described above. The present disclosure also embraces a viral vector comprising the isolated RNA described above or the isolated DNA encoding that RNA. In some embodiments, the viral vector is an adenovirus, adeno-associated virus, poxvirus, retrovirus, lentivirus, herpesvirus, alphavirus, or baculovirus.

The present disclosure also embraces a cell line containing the DNA described above. It further embraces a nanoparticle comprising the isolated RNA or DNA described above.

According to another aspect of the present disclosure, a method of inducing cell death is provided. The method involves administering a therapeutically effective amount of the isolated RNA or the isolated DNA described above, wherein the coding region codes for an antineoplastic agent.

According to another aspect of the present disclosure, both a viral vector and a target-specific translation activator are introduced into a cell.

According to another aspect of the present disclosure, a method of inducing an immunogenic response in a subject is provided. The method involves administering a therapeutically effective amount of any of the isolated RNA, the isolated DNA, the viral vector, the cell line, or the nanoparticle described above, and a target-specific translation activator to the subject.

According to another aspect of the present disclosure, a method of treating a viral infection in a subject is provided. The method involves administering a therapeutically effective amount of any of the isolated RNA, the isolated DNA, the viral vector, the cell line, or the nanoparticle described above to the subject.

In some embodiments, the isolated RNA is produced following in vivo administration of an isolated DNA encoding the isolated RNA. In some embodiments, the isolated RNA or the DNA encoding the isolated RNA is delivered as an inhaled nanoparticle or an inhaled viral vector.

In any of the embodiments described herein, the polypeptide of interest may be an antineoplastic protein.

In some embodiments, the isolated RNA or the DNA encoding the isolated RNA, and the target-specific translation activator are co-administered. In some embodiments, the isolated RNA or the DNA encoding the isolated RNA or the target-specific translation activator are administered via viral infection. In some embodiments, the subject is a human.

In some embodiments, any of the isolated RNA, the isolated DNA, the viral vector, or the nanoparticle described above is administered to a cell. In some embodiments, the cell is a human cell, an animal cell, or a plant cell. In some embodiments, the isolated RNA or the isolated DNA or the viral vector or the nanoparticle is administered ex vivo.

According to another aspect of the present disclosure, a method of generating a transgenic animal or plant is provided. The method includes inserting any of the isolated RNA, the isolated DNA, the viral vector, or the nanoparticle described above into said animal or plant. In some embodiments, the coding region of the RNA polynucleotide encodes an antiviral polypeptide of interest. In some embodiments, the transgenic animal or plant has increased resistance to viral infection. In some embodiments, the transgenic animal or plant is an avian, pig, fish, cow, horse, camel, dog, cat, mouse, rat, cotton rat, hamster, ferret, primate, or other commercially valuable animal or plant species.

In some embodiments, the polypeptide of interest comprises an antiviral polypeptide. In some embodiments, the transgenic animal or cell has increased resistance to viral infection.

According to another aspect of the present disclosure, a method of increasing the activation of an encrypted RNA is provided by complexing any of the isolated RNA described above with an RNA-binding protein. In some embodiments, the RNA-binding protein is a viral nucleocapsid protein or capsid protein. In some embodiments, the RNA-binding protein is a viral nucleocapsid protein or capsid protein of the target virus of the Encrypted RNA. In some embodiments, the viral nucleocapsid protein or capsid protein is obtained from an influenza virus, sarbecovirus, pneumovirus, paramyxovirus, henipavirus, or hepadnavirus.

Each of the limitations of the compositions and methods described in this disclosure may encompass various described embodiments. It is, therefore, anticipated that each of the limitations of the invention involving any one element or combinations of elements can be included in each aspect of the invention. This present disclosure is not limited in its application to the details of construction and the arrangement of components set forth in the following description or illustrated in the drawings.

### BRIEF DESCRIPTION OF THE DRAWINGS

The accompanying drawings are not intended to be drawn to scale. For purposes of clarity, the drawings are illustrative only and are not required for enablement of the disclosure. Not every component may be labeled in every drawing. In the drawings:

FIGS. 1A-1B show schematics of how some embodiments of encrypted RNAs function. FIG. 1A shows an encrypted RNA in the absence of a target-specific translation activator. FIG. 1B shows an encrypted RNA in the presence of a target-specific translation activator.

FIGS. 2A-2B show schematics comparing an encrypted RNA with an mRNA in the presence or absence of a translation activator of the encrypted RNA. FIG. 2A shows that the level of protein translation from the mRNA is not dependent on the presence of the translation activator in a cell. In contrast, FIG. 2B shows that the activation of the encrypted RNA is dependent on the presence of the target-specific translation activator in a cell.

FIGS. 3A-3C show schematics of some embodiments of therapeutic encrypted RNAs, which encode a therapeutic polypeptide of interest and for which the translation activator is provided by virus infection of a cell. FIG. 3A shows a schematic of some embodiments of therapeutic encrypted RNAs, wherein negligible levels of the therapeutic polypeptide of interest are translated in a cell in the absence of a translation activator such as viral infection. FIG. 3B is a schematic showing that, in some embodiments, viral infection of a cell in the absence of therapeutic encrypted RNA treatment can result in high levels of viral replication. FIG. 3C is a schematic showing that, in some embodiments, upon virus infection of a cell treated with a therapeutic encrypted RNA, increased translation of the therapeutic polypeptide of interest occurs.

FIG. 3C also shows that in some embodiments, a therapeutic polypeptide of interest is a secreted protein, for example a cytokine that induces an antiviral response after it binds to its receptor on the surface of a cell.

FIGS. 4A-4B describe and show experiments to test the level of activation of an encrypted RNA after infection of treated cells with a virus. FIG. 4A shows a schematic of the design of an experiment to test the level of activation of an encrypted RNA in treated cells when the cells are infected with different viral doses (multiplicities of infection, MOI). FIG. 4B shows influenza encrypted RNAs (Encrypted v2 and Encrypted v3) that are engineered from a prototype influenza encrypted RNA (Encrypted v1) to enable enhanced translation of the polypeptide of interest during influenza infection. FIG. 4B shows that, in some embodiments of an encrypted RNA, levels of the polypeptide of interest (GDura) can be increased by more than 10⁴× after contact of the encrypted RNA with a translation activator.

FIG. 5 shows that, in some embodiments, an encrypted RNA can be activated by a translation activator in the absence of virus infection. For example, in cells treated with an influenza encrypted RNA (ERNA-IAV-002-GDura), transfecting the cells with plasmids encoding influenza A polymerase proteins (PB1, PB2, and PA) and NP protein can substantially increase translation of the polypeptide of interest in the absence of virus infection.

FIG. 6 shows that an influenza A encrypted RNA can be activated by influenza A or influenza B strains. Levels of the polypeptide of interest encoded by the encrypted RNA (LMAX-LNP-formulated ERNA-IAV-002-GDura) are shown in the presence or absence of influenza A or B virus infections. In encrypted RNA-treated cells that are infected with influenza A or B viruses, levels of the polypeptide of interest can increase by approximately 10,000-100,000× (i.e., 4-5 log). Influenza strains shown: A/H1N1; A/H3N2; an influenza B strain from the “Yamagata” lineage; an influenza B strain from the “Victoria” lineage.

FIG. 7 shows that, in some embodiments, an influenza encrypted RNA (LMAX-LNP formulated ERNA-IAV-002-GDura) is not substantially activated by non-influenza viruses such as OC43-CoV, RSV or EMCV (e.g. translation of the polypeptide of interest is not substantially driven by nonspecific cellular immune responses to viral infection).

FIGS. 8A-8B shows that, in some embodiments, an influenza encrypted RNA can be substantially activated even when nucleoside-modified, 5′-monophosphorylated, or 5′-capped. FIG. 8A shows that a 5′-triphosphorylated influenza encrypted RNA (S158), a 5′-monophosphorylated influenza encrypted RNA (S159), or an influenza encrypted RNA with ~70% of uridine nucleotides modified to pseudouridine (S160), can be substantially activated by influenza A/PR8 infection. FIG. 8B shows that an influenza encrypted RNA (S158) retains the ability to be substantially activated in the presence of an influenza translation activator after incorporating a diversity of nucleotide modifications or 5′-capping. The influenza translation activator was provided by co-transfecting the treated cells with plasmids encoding PolA. In this drawing, +Flu means an encrypted RNA treated culture was co-transfected with 4 plasmids encoding PolA. In contrast, −Flu means an encrypted RNA treated culture was not co-transfected with plasmids encoding PolA.

FIG. 9 shows that, in some embodiments, an influenza encrypted RNA can be activated by influenza infection at least 13 weeks after treatment of cells with a DNA-encoded influenza encrypted RNA cassette (LVG04-ERNA-GDura).

FIGS. 10A-10B show that, in some embodiments, an influenza encrypted RNA can be activated by influenza infection multiple times after a single of treatment of cells with a DNA-encoded influenza encrypted RNA cassette (LVG04-ERNA-GDura). FIG. 10A shows the activation of the DNA-encoded influenza encrypted RNA cassette after an initial influenza infection. Notably, activation decays within 3 days of influenza infection (due to a reduction in viral titers). FIG. 10B shows that activation of the DNA-encoded influenza encrypted RNA cassette is restored after a second influenza infection of the cells 1 week later.

FIG. 11 shows that, in some embodiments, a therapeutic sarbecovirus encrypted RNA has antiviral efficacy against a virus encoding a translation activator of the encrypted RNA. Treatment of cells with an uncapped therapeutic encrypted RNA (LMAX-LNP-formulated uncapped ERNA-SARS2-101-hu_IFNB) elicited an approximately 3 log reduction of SARS-COV-2 generation-limited infection model with respect to untreated cells, and a more than 2-log reduction in virus level with respect to cells that received either an uncapped non-therapeutic encrypted RNA (LMAX-LNP-formulated uncapped ERNA-SARS2-101-GDura) or an LMAX-LNP-formulated uncapped mRNA encoding a GFP (a non-therapeutic protein).

FIG. 12 shows that, in some embodiments, a therapeutic encrypted RNA can provide efficacy against influenza infection at least thirteen weeks after treatment of cells with a DNA-encoded therapeutic influenza encrypted RNA cassette (LVG04-ERNA-hu_IFNB, labelled IFN-β). Efficacy of the DNA-encoded therapeutic influenza encrypted RNA cassette wherein human IFN-β is the polypeptide of interest is compared with efficacy of a DNA-encoded encrypted RNA cassette (LVG04-ERNA-GDura, labelled GDura) wherein GDura is the polypeptide of interest.

FIG. 13 shows a pairwise alignment of the L & R flanking sequences of some influenza antisense encrypted RNAs and highlights key nucleotide differences between the sequences. Sequences are written in DNA form and their conversion to RNA is also implied.

FIG. 14 shows that, in some embodiments, an influenza B encrypted RNA (pAT002-ERNA-IBV-001-GDura) comprised of the 5′ and 3′ vRNA termini of the HA segment of an influenza B vRNA can be activated by influenza A (H1N1 or H3N2 strains) or influenza B strains. FIGS. 6 and 14 collectively show that, in some embodiments, the same encrypted RNA can be activated by distinct translation activators, or, in some embodiments, distinct encrypted RNAs can be activated by the same translation activator.

FIG. 15 shows a schematic of some sarbecovirus sense encrypted RNAs.

FIGS. 16A-16B show that in cells treated with some sarbecovirus sense encrypted RNAs, translation of a polypeptide of interested is increased when the cells are infected with SARS-COV-2. The encrypted RNAs shown are: ERNA-SARS2-101-GDura (labelled “WT”), ERNA-SARS2-102-GDura (labelled “N250”), ERNA-SARS2-109-GDura (labelled “ATG_HP45”), ERNA-SARS2-110-GDura (labelled “ATG_HP60”), and ERNA-SARS2-105-GDura (labelled “N250-ATG_HP45”). For each tested encrypted RNA, FIG. 16A shows levels of the polypeptide of interest at 24 hours post-infection with SARS-COV-2. For each tested encrypted RNA, FIG. 16B shows levels of the polypeptide of interest at 48 hours post-infection with SARS-COV-2.

FIG. 17 shows, in cells treated with some sarbecovirus sense encrypted RNAs, a normalized increase in translation of the polypeptide of interest can occur when cells are infected with SARS-COV-2, as compared to translation of the polypeptide of interest in the absence of SARS-COV-2 infection. Shown are the same encrypted RNAs as in FIG. 16. For each encrypted RNA, FIG. 17 shows the level of activation at both 24 and 48 hours after SARS-COV-2 infection, normalized to the level of activation in the absence of SARS-COV-2.

FIGS. 18A-18B show activation of some sarbecovirus encrypted RNAs after SARS2-GL infection of treated cells. FIG. 18A shows the dose-dependent activation of a sarbecovirus encrypted RNA (LMAX-LNP-formulated ERNA-SARS2-101-GDura) in cells infected with SARS-COV-2 (GL) at two different MOI (1× and 10×). FIG. 18B shows that a sarbecovirus encrypted RNA can also be developed from L and R regions that are derived from SARS-COV-1. The activation of LMAX-LNP-formulated ERNA-SARS2-101-GDura and LMAX-LNP-formulated ERNA-SARS1-101-GDura is compared in cells infected with SARS-COV-2 (GL). In this example, the SARS-COV-1 derived encrypted RNA is activated as well or better than the SARS-COV-2 derived encrypted RNA in a SARS-COV-2 infection.

FIG. 19 shows a schematic of some embodiments of sarbecovirus antisense encrypted RNAs that rely on the addition of an IRES sequence to increase translation of a polypeptide of interest.

FIGS. 20A-20B show that, in cells treated with some sarbecovirus antisense encrypted RNAs, translation of the polypeptide of interest can be increased when the cells are provided with a variety of sarbecovirus-derived translation activators. The cells were treated with one of 3 DNA-encoded sarbecovirus antisense encrypted RNA cassettes and transfected with either: (i) no additional plasmids (“none”); (ii) plasmids producing SARS-COV-2 nsp7, nsp8, nsp12 polypeptides; (iii) plasmids producing SARS-COV-2 nsp7, nsp8, nsp12, and Nucleoprotein (N) polypeptides; (iv) a multigenic BAC expression plasmid which drives SARS-COV-2 orf1ab production from a constitutive minimal HCMV IE2 promoter and separately drives SARS-COV-2 Nucleoprotein (N) production from an EF1a promoter (“minirep”); (v) a SARS-COV-2 BAC which produces a SARS-COV-2 genome competent for orf1ab production but deficient for all structural proteins except N (“S2-trans”). FIG. 20A shows the level of activation for each encrypted RNA and transfection pool at 24 hours post-transfection.

FIG. 20B shows the level of activation for each encrypted RNA and transfection pool at 48 hours post-transfection.

FIG. 21 shows a schematic of some embodiments of RSV encrypted RNAs (RSV means Respiratory Syncytial Virus).

FIG. 22 shows that, in cells treated with an RSV encrypted RNA, translation of the polypeptide of interest (GDura) can be substantially increased by infection of the cells with RSV.

FIG. 23 shows activation of a therapeutic RSV encrypted RNA encoding human IFN-β as the polypeptide of interest in the presence or absence of RSV infection. The level of production of human IFN-β is measured by ELISA.

FIGS. 24A-24H provide summary drawings showing the activation of some encrypted RNAs by translation activators comprising viral RNA dependent polymerases. FIG. 24A shows the activation of a sarbecovirus encrypted RNA by a panel of different sarebcovirus variants. FIG. 24B shows the activation of an influenza encrypted RNA in the presence of influenza A and B translation activators. FIG. 24C shows the activation of a henipavirus encrypted RNA in the presence of Nipah and Hendra translation activators. FIG. 24D shows the activation of a filovirus encrypted RNA in the presence of a Zaire ebolavirus (ZEBOV) polymerase complex (labelled “EBOV” here). FIG. 24E shows the activation of an RSV encrypted RNA in in the presence of RSV translation activators. FIG. 24F shows activation of an HPIV1 encrypted RNA by HPIV1 infection. FIG. 24G shows activation of an HPIV3 encrypted RNA by HPIV3 infection. FIG. 24H shows activation of an HMPV encrypted RNA by HMPV infection. More generally, encrypted RNAs were developed against viruses with divergent polymerase proteins or replication cycles: e.g., influenza (−sense RNA viruses, nuclear replication); sarbecoviruses (+sense RNA viruses, cytoplasmic replication); RSV (−sense RNA viruses, cytoplasmic replication).

FIGS. 25A-25B show a therapeutic RSV encrypted RNA that can confer efficacy against an RSV infection (strain A2). FIG. 25A shows micrographs of infections of HEp-2 cells by an RSV (labelled with a red fluorescent reporter protein) when the cells are: untreated (left panel), treated with a therapeutic RSV encrypted RNA encoding human IFN-β as the polypeptide of interest (middle panel), or treated with a control RSV encrypted RNA encoding a luciferase as the polypeptide of interest (right panel). FIG. 25B shows that a therapeutic RSV encrypted RNA (encoding a human IFN-β protein) can reduce RSV viral levels by approximately 10-100×(1-2 log) in HEp-2 cells, as quantified by a viral plaque assay.

FIG. 26 shows the antiviral efficacy of an LMAX-LNP-formulated RSV antisense encrypted RNA against RSV (strain A2).

FIGS. 27A-27C show that a therapeutic sarbecovirus encrypted RNA can be effective at reducing the viral loads of multiple sarbecovirus variants in Vero-hACE2-TMPRSS2 cells. FIG. 27A shows that the therapeutic sarbecovirus encrypted RNA is effective at reducing the viral level of a Delta variant of SARS-COV-2, in comparison to viral levels in cells treated with a control non-therapeutic sarbecovirus encrypted RNA (encoding GDura) or a control mRNA encoding a GFP. FIG. 27B shows that the therapeutic sarbecovirus encrypted RNA is effective at reducing the viral level of an Omicron variant of SARS-COV-2. FIG. 27C shows that the therapeutic sarbecovirus encrypted RNA is effective at reducing the viral level of an ancestral (WA1) variant of SARS-COV-2. Viral loads were measured by plaque assay.

FIG. 28 shows a capped therapeutic sarbecovirus encrypted RNA that does not provide substantial efficacy against influenza, which does not provide a translation activator for the encrypted RNA.

FIGS. 29A-29B show that a capped therapeutic sarbecovirus encrypted RNA encoding mouse IFN-β can be safe and effective against SARS-COV-2 in mice, when administered to mice prophylactically. Groups of mice were provided with one of 3 treatments (ERNA-SARS2-001-m_IFNB, ERNA-SARS2-001-GDura, or a vehicle-control alone) and then infected with a lethal dose of a mouse-adapted variant of SARS-COV-2 (MA30). FIG. 29A shows mean body weight loss over time for each group of tested mice. FIG. 29B shows Kaplan-Meier survival curves for each group of tested mice.

FIG. 30 shows that an encrypted RNA delivered as a circular RNA or an encrypted RNA with additional terminal flanking sequences can be activated by viral infection.

FIG. 31 shows that, in some embodiments, treating cells with an encrypted RNA that incorporates modified nucleotides can, in the absence of a translation activator, reduce the background levels of encrypted RNA immunogenicity or the levels of translation of the polypeptide of interest.

FIG. 32 shows a schematic of some DNA-encoded encrypted RNA cassettes delivered using viral (e.g. lentiviral) vectors.

FIG. 33 shows that DNA cassettes incorporating enhanced Pol I terminator sequences can produce RNA transcripts without undesired additional 3′ nucleotides within the terminator sequences, as measured by 3′-RACE.

FIG. 34 shows that, in some embodiments, RSV encrypted RNAs with 5′ terminal modifications (e.g. a 5′-monophosphate) can be efficiently activated by a translation activator provided by RSV infection.

FIG. 35 shows that a 5′ terminal phosphate of the encrypted RNA is not required for activation by RSV infection.

FIG. 36 shows that, in some embodiments, RSV antisense encrypted RNAs can be activated by RSV infection up to at least 5 days after treatment of cells with a single dose of LNP-formulated encrypted RNA. Shown are both an LNP-formulated 5′-triphosphorylated RSV encrypted RNA and an LNP-formulated 5′-monophosphorylated RSV encrypted RNA administered to cells at either 250 ng or 100 ng doses on Day 0.

FIG. 37 shows that, in some embodiments, RSV antisense encrypted RNA can be activated by multiple strains of human RSV, including A2 or B1, when the 5′ end of the RSV antisense encrypted RNA is monophosphorylated or triphosphorylated.

FIG. 38 shows that, in some embodiments, nucleoside-modified RSV encrypted RNAs prepared via in vitro transcription can be activated via infection with RSV.

FIG. 39 shows a subset of the data in FIG. 37 highlighting that, in some embodiments, an RSV antisense encrypted RNA can be activated by RSV infection up to at least 5 days after treatment of cells with a single dose of LNP-formulated encrypted RNA.

FIGS. 40A-40B show that, in some embodiments, changing the TRS sequences present in sarbecovirus encrypted RNAs does not substantially affect encrypted RNA activation. FIG. 40A shows that SARS2-GL can substantially activate a sarbecovirus encrypted RNA possessing a different TRS (“non-cognate TRS”) than the viral genome. FIG. 40B shows that SARS2-GL can substantially activate a sarbecovirus encrypted RNA possessing the same TRS (“cognate TRS”) as the viral genome. FIGS. 40A-40B further show that, in the absence of a translation activator, the level of background translation of the polypeptide of interest of an encrypted RNA can depend on the presence or absence of a 5′-Cap on the encrypted RNA.

FIG. 41 shows that, in some embodiments, RSV sense encrypted RNAs or RSV antisense encrypted RNAs can be activated by RSV infection. The level of background translation of the polypeptide of interest can depend on the sequence of the encrypted RNA (e.g. sense or antisense).

FIG. 42 shows that, in some embodiments, translation activators of RSV encrypted RNAs can be provided via viral infection or as polynucleotide sequences encoding individual proteins (e.g. absent viral infection).

FIG. 43 shows that, in some embodiments, RSV encrypted RNAs can be transmitted to new cells via RSV infection and that the transmitted encrypted RNAs can be activated by RSV infection in these new cells. FIG. 43 additionally shows that, in the absence of RSV infection, some translation activators of RSV encrypted RNAs (e.g., plasmids encoding RSV N, P, M2-1, and L proteins) are not sufficient to enable sustained transmission of an RSV encrypted RNA.

FIG. 44A shows that, in some embodiments, a DNA-encoded encrypted RNA can be activated by a targeted viral infection to produce therapeutic polypeptide of interest weeks after treatment, when virus infection provides the translation activator.

FIG. 44B, shows that, in some embodiments, treatment of cells with a DNA-encoded encrypted RNA encoding a therapeutic polypeptide (human IFN-beta) is effective at inhibiting viral infection, while treatment of cells with an analogous DNA-encoded encrypted RNA not encoding a therapeutic polypeptide is ineffective at preventing virus replication. FIG. 44C shows that immunocompetent cells (A549) can be effectively treated by transduction with a lentiviral vector encoding a DNA-encoded encrypted RNA, with the cassette persisting multiple weeks after delivery (>14 days).

FIG. 45 shows that, in some embodiments, a hepadnavirus encrypted RNA (ERNA-HBV-105-GDura) can be substantially activated by providing a translation activator, comprising the core protein of HBV (Huh-7 NTCP cells).

FIG. 46 shows that, in some embodiments, a sarbecovirus encrypted RNA can be substantially activated after infection of treated cells by any of a panel of SARS-CoV-2 variants, including: USA/WA1/2020 (“Ancestral” or “WA1”), Beta (B.1.351), Delta (B.1.617.2), an Omicron BA.4 isolate, an Omicron BA.5 isolate, or “MA30”. In contrast, the sarbecovirus encrypted RNA was not substantially activated in cells infected with influenza A/PR8 alone.

FIGS. 47A-47B show that a capped therapeutic sarbecovirus encrypted RNA encoding mouse IFN-lambda2 can be safe and effective against SARS-COV-2 in mice, when administered to mice prophylactically. Groups of mice were provided with one of 3 treatments (ERNA-SARS2-001-m_IFN_L2, ERNA-SARS2-001-GDura, or a vehicle-control alone) and then infected with a lethal dose of MA30. FIG. 47A shows mean body weight loss over time for each group of tested mice. FIG. 47B shows Kaplan-Meier survival curves for each group of tested mice.

FIGS. 48A-48B show that, in some embodiments, a control therapeutic RSV encrypted RNA encoding mouse IFN-lambda2 is not effective against SARS-COV-2 in mice, when administered to mice prophylactically. Groups of mice were provided with one of 3 treatments (ERNA-SARS2-001-m_IFN_L2, ERNA-RSV-005-m_IFN_L2, or a vehicle-control alone) and then infected with a lethal dose of MA30. FIG. 48A shows mean body weight loss over time for each group of tested mice. FIG. 48B shows Kaplan-Meier survival curves for each group of tested mice.

FIGS. 49A-49B show that a capped therapeutic sarbecovirus encrypted RNA encoding mouse IFN-lambda2 can be safe and effective against SARS-COV-2 in mice, when administered to mice therapeutically after infection with MA30. Groups of mice were infected with a lethal dose of MA and then provided with one of 3 treatments (ERNA-SARS2-001-m_IFN_L2, ERNA-SARS2-001-GDura, or a vehicle-control alone). FIG. 49A shows mean body weight loss over time for each group of tested mice. FIG. 49B shows Kaplan-Meier survival curves for each group of tested mice.

FIGS. 50A-50B show that a therapeutic sarbecovirus encrypted RNA encoding hamster IFN-lambda3 can be safe and effective against SARS-COV-2 in Syrian hamsters. Groups of hamsters were provided with one of 3 treatments (ERNA-SARS2-001-ham_IFN_L3, ERNA-SARS2-001-GDura, or a vehicle-control alone) and then infected with SARS-COV-2 WA1. FIG. 50A shows H&E staining from the lungs, liver and heart obtained from treated animals at necroscopy. FIG. 50B shows infectious viral load from lung homogenates and oropharyngeal (OP) swabs for the therapeutic sarbecovirus encrypted RNA and control treatments. Samples were taken at necroscopy 3 days post-infection and viral load was quantified via plaque assay.

FIG. 51A shows a simplified schematic of an experiment to test the persistence of an RSV encrypted RNA in treated cells. FIG. 51B shows the result of one such experiment in cells treated with ERNA-RSV-005-GDura on Day 0. RSV infection 14 days later resulted in activation levels >100× above background when cells were co-transfected with RSV N and RSV P on Day 0. In contrast, in cells that received the encrypted RNA alone (i.e. without N, P) on Day 0, no encrypted RNA could be activated 14 days after treatment. Thus, the persistence of an encrypted RNA in treated cells can be substantially increased by complexing the encrypted RNA with RNA-binding proteins, such as nucleoproteins.

FIG. 52 shows that, in some embodiments, LNP-encapsulated ERNA-RSV-005-GDura can be substantially activated (in the absence of viral infection) via co-transfection with plasmids or mRNAs encoding RSV proteins L, N, M2-1, and P (together a translation activator of the RSV encrypted RNA). Whether plasmids or mRNAs are co-transfected into cells, activation of the encrypted RNA was increased by ~3 logs within 48 hours when polynucleotides encoding L, N, M2-1, and P are provided to the cells.

FIG. 53 shows that, in some embodiments, an LNP-formulated RSV encrypted RNA can be substantially activated by a panel of RSV A and B variants, including clinical isolates, but is not activated by non-RSV species.

FIGS. 54A-54B show activation of some RSV encrypted RNAs in the presence or absence of RSV infection in human primary airway cells. FIG. 54A shows that human primary airway cells treated with ERNA-RSV-005-GDura exhibited an ~2 log increase in translation of the GDura polypeptide of interest in the presence of RSV infection. FIG. 54B shows an analogous experiment with an RSV encrypted RNA encoding human IFN-β as the polypeptide of interest, where translation of the therapeutic polypeptide was increased by ~300 μg/ml (as quantified by ELISA) in the presence of RSV infection.

FIGS. 55A-55B show activation and antiviral efficacy of some RSV encrypted RNAs in the presence or absence of RSV infection in HEp-2 cells. FIG. 55A shows an encrypted RNA encoding human IFN-β as the polypeptide of interest, where translation of the therapeutic polypeptide was increased by ~2000 μg/ml (as quantified by ELISA) in the presence of RSV infection. FIG. 55B shows an approximately 2 log reduction in RSV viral load in Hep-2 cells treated with a therapeutic RSV encrypted RNA encoding human IFN-β protein as the polypeptide of interest (ERNA-RSV-005-hu_IFNB). Notably, the viral load knockdown in comparison to a matched control non-therapeutic encrypted RNA can be even more pronounced when a therapeutic encrypted RNA is optimized to lack a 5′-triphosphate and to thereby reduce off-target immunogenicity.

FIGS. 56A-56B show that encrypted RNA scaffolds can be used to encode multiple polypeptides of interest, which can be administered simultaneously to cells (e.g. to provide combination therapies of multiple therapeutic polypeptides against the same disease). FIG. 56A shows Vero-E6-hACE2+ORF3a/E hACE cells treated with a sarbecovirus sense encrypted RNA encoding GDura, a sarbecovirus sense encrypted RNA encoding IFN-β, or both encrypted RNAs together. Notably, when cells are treated with both sarbecovirus encrypted RNAs and infected with SARS2-GL, both polypeptides of interest (in this case, GDura and IFN-β) are substantially increased at the same time. FIG. 56B shows HEp-2 cells treated with an RSV antisense encrypted RNA encoding GDura, an RSV antisense encrypted RNA encoding IFN-β, or both encrypted RNAs together. Notably, when cells are treated with both RSV encrypted RNAs and infected with RSV, both polypeptides of interest (in this case, GDura and IFN-β) are substantially increased at the same time.

FIGS. 57A-57B show that multiple encrypted RNA scaffolds can be used to encode the same polypeptide of interest (e.g. to enable activation of a therapeutic protein against multiple viral infections simultaneously). FIG. 57A shows an experiment performed in cells treated with both a sarbecovirus encrypted RNA and an RSV encrypted RNA and infected with either RSV, SARS-COV-2 (GL), or both viruses. Notably, when the cells are infected with either virus, the corresponding encrypted RNA activates, and when cells are infected by both viruses simultaneously, both encrypted RNAs activate simultaneously. FIG. 57B shows an analogous experiment where cells are treated with both an RSV encrypted RNA and a DNA vector (lentivirus) encoding an influenza encrypted RNA. An analogous result is seen to that in FIG. 57A, namely that when cells are infected with either influenza or RSV, the corresponding encrypted RNA activates.

FIGS. 58A-58B show the plug-and-play capability of both an influenza encrypted RNA scaffold and an RSV encrypted RNA scaffold, for example to encode immunomodulatory proteins as polypeptides of interest. FIG. 58A shows an influenza encrypted RNA scaffold encoding human IL-12 (ERNA-IAV-002-hu_IL_12) or mouse IL-2 (ERNA-IAV-002-mIL_2) as the polypeptide of interest. When co-delivered with an influenza translation activator, the encrypted RNA scaffold can produce more immunomodulatory protein than an mRNA directly encoding the immunomodulatory proteins. FIG. 58B shows an RSV encrypted RNA encoding an anti-inflammatory protein (IL-1RN) as the polypeptide of interest. When treated cells are infected with RSV, the encrypted RNA can similarly translate higher levels of IL-1RN than an mRNA encoding IL-1RN.

FIG. 59 shows that, in some embodiments, a sarbecovirus encrypted RNA, ERNA-SARS2-101-GDura, incorporating modified nucleotides can be substantially activated by SARS2-GL. The figure shows that incorporation of 1-3% N6-methyladenosine (m6a) had no significant effect on the activation of a sarbecovirus encrypted RNA when the cells were infected with SARS-COV-2 GL. Further, when ERNA-SARS2-101-GDura was formulated with 30-60% of uridine nucleotides modified to pseudouridine, the encrypted RNA was activated similarly or up to approximately 1.5-fold higher than a matched ERNA-SARS2-101-GDura construct formulated with 100% unmodified U.

FIG. 60 shows that, in some embodiments, nucleoside-modification of an encrypted RNA (ERNA-SARS2-101-GDura) can lower immunogenicity of the encrypted RNA when delivered to cells in the absence of a translation activator. FIG. 60 also shows that, in some embodiments, immunogenicity of the encrypted RNA can be reduced by HPLC-purification or nucleoside-modification or both HPLC-purification and nucleoside-modification.

FIG. 61 shows that an antisense RSV encrypted RNA can be modified with a 5′-cap without losing the ability to be substantially activated by RSV infection. Notably, capping of the RSV encrypted RNA utilized an additional three-nucleotide AGG sequence added at the 5′-end of the L-region of the encrypted RNA. Capped and uncapped RSV encrypted RNAs demonstrated similar activation in response to RSV infection.

FIG. 62 shows that, in some embodiments, an RSV antisense encrypted RNA can incorporate up to 100% modified nucleotides without losing the ability to be substantially activated by RSV infection. RSV encrypted RNAs can incorporate at least up to 30% N6-methyladenosine (“m6a”), up to 100% 5-methylcytidine (“5-meC”), and up to 70% 5-methoxyuridine (“5-moU”) without substantially reduced activation (measured at 72 h post infection) in response to RSV infection. Unmodified is 100% uridine.

FIG. 63 further demonstrates that the RSV encrypted RNA incorporating a combination of two modified nucleotides such as 10% N6-meA and 70% 5-meOU can be substantially activated by RSV infection (measured at 48 h post RSV infection).

FIG. 64 shows that, in some embodiments, a nucleoside-modified RSV antisense encrypted RNA (ERNA-RSV-008-GDura) is significantly less immunogenic than an unmodified RSV encrypted RNA when provided to cells without the translation activator (as measured by an interferon-stimulated gene reporter, IRF, in A549 Dual cells).

FIG. 65 shows additional embodiments in which a nucleoside-modified RSV antisense encrypted RNA (ERNA-RSV-008-GDura) is significantly less immunogenic than nonmodified RSV encrypted RNA when provided to cells without the translation activator (as measured by an interferon-stimulated gene reporter, IRF, in A549 Dual cells). Nucleoside-modifications include replacement of uridine with 5-methoxyuridine (“methoxy” or “MeO”), or a complete replacement of uridine with a binary mixture of N1-methylpseudouridine (“m1”) and 5-methoxyuridine (“MeO”). The ratio of the binary mixture is indicated by numerals separated by a colon—e.g., 30% N1-methylpseudouridine and 70% 5-methoxyuridine is indicated by “30:70 m1-meO”.

FIG. 66 shows that, in some embodiments, nucleoside-modified encrypted RNAs can be activated in the presence of a translation activator. Some encrypted RNAs tested for compatibility with nucleoside-modification were: an influenza encrypted RNA (ERNA-IAV-002-GDura), a sarbecovirus (“SARS-2”) encrypted RNA (ERNA-SARS2-101-GDura), an RSV encrypted RNA (ERNA-RSV-008-GDura), an HPIV1 encrypted RNA (ERNA-HPIV1-002-GDura), an HPIV3 encrypted RNA (ERNA-HPIV1-003-GDura), an HMPV encrypted RNA (ERNA-HMPV-003-GDura), a henipavirus (“NiV”) encrypted RNA (ERNA-NiV-001-GDura), a henipavirus (“HeV”) encrypted RNA (ERNA-HeV-001-GDura), or a filovirus (“ZEBOV”) encrypted RNA (ERNA-ZEBOV-001-GDura).

FIG. 67 shows that, in some embodiments, encrypted RNAs can be nucleoside-modified with more than one class of nucleoside and continue to retain activation by a translation activator. An RSV encrypted RNA (ERNA-008-GDura) was nucleoside-modified by A-modification (e.g. 10% N6-methyladenosine), C-modification (e.g., 100% 5-methylcytidine), U-modification (N1-methylpseudouridine or 5-methyoxyuridine or both) or by more than one class of modification. Activation values are reported as a percentage of fthe activation of the nonmodified encrypted RNA.

### DETAILED DESCRIPTION

### Definitions

In order that the present disclosure can be more readily understood, certain terms are first defined. As used in this specification, except as otherwise expressly provided herein, each of the following terms shall have the meaning set forth below. Additional definitions are set forth throughout the specification.

The use of the alternative (e.g., “or”) should be understood to mean either one, both, or any combination thereof of the alternatives. As used herein, the term “and/or” has the same meaning as “or”.

As used herein, the indefinite articles “a” or “an” or “some” should be understood to refer to “one or more” of any recited or enumerated component. As such, the terms “a”, “an”, “some”, “one or more”, and “at least one” can be used interchangeably.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition that “comprises,” “has” or “includes” one or more features is not limited to possessing only those one or more features and can cover other unlisted features. It is understood that wherever aspects are described herein with the language “comprising”, “having”, or “including”, otherwise analogous aspects described in terms of “consisting of” or “consisting essentially of” are also provided.

The use of any and all examples, or exemplary language (e.g. “such as”) provided with respect to certain embodiments, herein is intended merely to better illuminate the present disclosure and does not pose a limitation on the scope of the present disclosure otherwise claimed. This disclosure is not limited to the particular methodology, protocols, and reagents, etc., described herein and as such can vary. No language in the specification should be construed as indicating any non-claimed element as essential to the practice of the present disclosure. The terminology used herein is for the purpose of describing particular embodiments only, and is not intended to limit the scope of the present disclosure, which is defined solely by the claims.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2^{nd} ed., 2002, CRC Press; The Dictionary of Cell and Molecular Biology, 3^{rd} ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “about”, “substantially”, “approximately”, or “comprising essentially of” refer to a value or composition that is within an acceptable error range for the particular value or composition as determined by one of ordinary skill in the art, which will depend in part on how the value or composition is measured or determined, e.g., on the limitations of the measurement system. For example, “about”, “substantially”, “approximately”, or “comprising essentially of” can mean within 1 or more than 1 standard deviation per the practice in the art. Alternatively, “about,” “substantially”, “approximately”, or “comprising essentially of” can mean a range of up to 20%. Furthermore, particularly with respect to biological systems or processes, the terms can mean up to 5-fold or up to 10-fold of a value. When particular values or compositions are provided in the application and claims, unless otherwise stated, the meaning of “about” “substantially”, “approximately”, or “comprising essentially of” should be assumed to be within an acceptable error range for that particular value or composition.

As used herein, any concentration range, percentage range, ratio range, or integer range is to be understood to include the value of any integer within the recited range and, when appropriate, fractions thereof (such as one tenth or one hundredth of an integer), unless otherwise indicated. In addition, all ranges are intended to expressly include the boundaries of the range individually. For clarity, the range 3-6 is intended to include individually 3, 4, 5 and 6 as well as any fraction within that range.

As used herein, a “target-specific translation activator” is one or more polypeptides that directs synthesis of a coding region of an encrypted RNA, which coding region comprises a coding sequence that encodes a polypeptide of interest that is translated at increased levels when the target-specific translation activator contacts the encrypted RNA. As used herein, “translation activator” means “target-specific translation activator”. In some embodiments, the target-specific translation activator is a polymerase. In some embodiments, the polymerase is an RNA-dependent RNA polymerase (RdRp). In some embodiments, the polymerase is an RNA-dependent DNA polymerase (RdDp, also referred to as a reverse transcriptase (RT)).

As used herein, an “encrypted RNA” is an isolated ribonucleic acid (RNA) polynucleotide, comprising: (a) a “coding region” which comprises a coding sequence that encodes a polypeptide of interest; and (b) “template regions” for binding a target-specific translation activator; wherein the target-specific translation activator directs transcription of mRNA that is distinct from the isolated RNA, and wherein translation of the polypeptide of interest is increased in a cell containing said RNA polynucleotide when the RNA polynucleotide is contacted in said cell with the target-specific translation activator. As used herein, a “polypeptide of interest” or “protein of interest” is a polypeptide encoded within a coding sequence of a coding region of an encrypted RNA according to the present disclosure.

The template regions are comprised of two distinct regions, a left flanking region (“L region”) of a virus and a right flanking region (“R region”) of the virus. The L region is 5′ to and contiguous with the coding region and the R region is 3′ to and contiguous with the coding region. Examples of L and R regions of various viruses as well as variants are provided in the Sequence Listing and in the Tables and Examples below. In some embodiments, the L and the R regions of a virus each do not contain a polynucleotide sequence encoding a polypeptide. In some embodiments, the L or the R region can contain a polynucleotide sequence encoding a polypeptide, which polypeptide is homologous to the the virus. If the L or the R region contain(s) a polynucleotide sequence, then that polynucleotide sequence contributes to the interaction of the L or R region, as appropriate, with the translation activator.

A coding region comprises one or more coding sequences. In some embodiments, the coding region contains two or more (e.g., 2, 3, 4, or more) coding sequences. In some embodiments, the coding region contains one coding sequence. In addition, a coding region may contain one or more non-coding sequences. Typically, a coding region contains a 5′ untranslated region (5′ UTR), a coding sequence, and a 3′ untranslated region (3′ UTR).

A “coding sequence” is a sequence of nucleotides which encodes the complete amino acid sequence of at least one polypeptide of interest. In some embodiments, the coding sequence encodes two or more (e.g., 2, 3, 4, or more) polypeptides. In some embodiments, the coding sequence encodes one polypeptide. As used herein, “a polypeptide of interest” is a polypeptide encoded by the coding sequence of a coding region. In some embodiments, the coding sequence of a coding region encodes a polypeptide that is heterologous to the virus from which the L and R regions of the encrypted RNA are derived. As used herein, “heterologous to the virus” means the coding sequence encodes a polypeptide that is not naturally found in the species of the virus (not a native polypeptide). As used herein, “homologous to the virus” means the coding sequence encodes a polypeptide that is naturally found in the species of the virus. A homologous sequence of a virus may also be referred to “native” to the virus. Classification of viral species is according to internationally accepted standards established by the International Committee on Taxonomy of Viruses (“ICTV”). The coding sequence is comprised of a series of three-nucleotide units, known as codons. The first three nucleotides of a coding sequence, the “start codon”, initiate translation of the polypeptide(s) of interest and typically encode for methionine or N-formylmethionine. An example start codon is “atg”. The final three nucleotides of a coding sequence, the “stop codon”, encode a stop codon or termination codon which terminates translation elongation of the polypeptide(s) of interest. Some examples of stop codons are “tag” (amber stop codon), “taa” (ochre stop codon), and “tga” (opal stop codon).

A “non-coding sequence” is a contiguous sequence of nucleotides which does not contain a coding sequence and does not encode a polypeptide. Non-coding sequences can be used to alter the expression of a polypeptide of interest. Non-limiting examples of non-coding sequences include 5′-untranslated region (UTR), 3′-UTR, promoters, introns, ribozymes, riboswitches, ribosome binding sites, Kozak sequences, Shine-Dalgarno sequences, Internal Ribosomal Entry Site(s) (IRES), poly-adenylation signals, poly-A sequences, microRNA binding sites, and other regulatory elements. As mentioned above, in some embodiments either the L or R region, or both of the L and the R regions consist of non-coding sequences. In some embodiments, the L or the R region can contain a polynucleotide sequence encoding one or more polypeptide, which polypeptide is homologous to the the virus.

A “5′-UTR of a coding sequence” or “5′ untranslated region of a coding sequence” is a non-coding sequence located adjacent to and contiguous with the 5′ start codon of a coding sequence. When a coding sequence is the first coding sequence 3′ of an L region, the 5′-UTR of the coding sequence begins at the first nucleotide of the first 5′ non-coding sequence in the coding region and ends one nucleotide before the start codon of the coding sequence. If there are two (or more) coding sequences in the coding region, then the coding sequences can be separated by untranslated regions. When a coding sequence is not the first coding sequence 3′ of an L region, there can be a second 5′-UTR for the second coding sequence (a third 5′-UTR for the third coding sequence, and so on), which separates the coding sequences from one another. The 5′-UTR of a coding sequence may comprise elements for controlling gene expression, also called regulatory elements. Such regulatory elements include, for example, ribosomal binding sites, Kozak sequences, Shine-Dalgarno sequences, ribozymes, riboswitches, promoters, microRNA binding sites, or IRES elements.

A “3′-UTR of a coding sequence” or “3′ untranslated region of a coding sequence” is a non-coding sequence located adjacent to and contiguous with the 3′ stop codon of a coding sequence. When a coding sequence is the first coding sequence adjacent to the 5′ end of an R region, the 3′-UTR of the coding sequence begins at the first nucleotide following the stop codon of the coding sequence and ends at the last 3′ nucleotide of the coding region before the 5′ end of the R region. If there are two (or more) coding sequences in the coding region, then the first and the second coding sequences can be separated by untranslated regions. A first 3′-UTR of the first coding sequence can separate the first coding sequence from the next adjacent coding sequence nearer the R region (and a second 3′UTR of a second coding sequence can separate the second coding sequence from the next adjacent coding sequence nearer the R region, and so on). The 3′-UTR of a coding sequence may comprise one or more elements for controlling gene expression, also called regulatory elements. Such regulatory elements include, for example, ribozymes, micro RNA binding sites, poly(A) sequences, and polyadenylation signals.

Translation of the polypeptide of interest or protein of interest in an encrypted RNA of the present disclosure is increased when the encrypted RNA contacts/interacts with a target-specific translation activator of the encrypted RNA. Such interaction initiates activity of the target-specific translation activator.

In some embodiments, the polypeptide of interest is a “therapeutic polypeptide”. A therapeutic polypeptide, exemplified in greater detail below, is a polypeptide that treats or ameliorates one or more symptoms of a disease or condition in a subject. In some embodiments the treatment is of an existing condition. In some embodiments, the treatment is prophylactic treatment. In some embodiments, the therapeutic polypeptide encoded by an encrypted RNA is heterologous to the virus from which the L and R regions of the encrypted RNA are derived. In some embodiments, the coding sequence for a therapeutic polypeptide does not naturally occur in the same nucleotide position in a viral genome. In some embodiments, the therapeutic polypeptide is an immunomodulatory protein, such as a human immunomodulatory protein known to exert an activity on the human immune system. Examples of immunomodulatory proteins include proteins such as a chemokine, a cytokine, an interleukin, a factor, an antibody, an immune checkpoint inhibitor, or an aptamer. A therapeutic polypeptide in some embodiments is a native human protein or an analog of a human protein (e.g., a truncated version of the protein or variant of the protein having one or more amino acid substitutions). In some embodiments, the therapeutic polypeptide is an antigen, including a self antigen including a cancer antigen or an antigen present in a pathogen.

A “therapeutic polypeptide of interest”, a “therapeutic polypeptide”, or a “therapeutic protein” has an advantageous effect on the condition or disease state of a subject when administered to the subject in a therapeutically effective amount. In some aspects, a therapeutic polypeptide has curative or palliative properties and may be administered to ameliorate, relieve, alleviate, reverse, delay onset of or lessen the severity of one or more symptoms of a disease or disorder. A therapeutic polypeptide may have prophylactic properties and may be used to delay or prevent the onset of a disease or to lessen the severity of such disease or pathological condition. The term therapeutic polypeptide includes entire proteins or polypeptides, and can also refer to active fragments thereof. It can also include active analogs of a peptide or protein. A pharmaceutically active peptide or protein can also be referred to as a therapeutic peptide or protein.

In some embodiments, the polypeptide of interest is a polypeptide that when administered to a particular subject, does not provoke or induce a medically significant antigen-specific response to the polypeptide of interest. In some embodiments, the polypeptide of interest is an immunostimulatory polypeptide. In some embodiments, the polypeptide of interest is a polypeptide that when administered to a particular subject, provokes or induces a medically significant antigen-specific response to the polypeptide of interest. In some embodiments, the polypeptide of interest is an immunosuppressive polypeptide. In some embodiments, the polypeptide of interest that when administered to a particular subject, provokes or induces a medically significant immunosuppressive immune response or inhibits or prevents an immunostimulatory or inflammatory immune response.

In some embodiments, the polypeptide of interest is a reporter polypeptide. Examples of reporter polypeptides are provided in the Examples and are well known to those of ordinary skill in the art.

As used herein, “activation” or “activate” describe the process or action or series of processes or series of actions by which translation of a polypeptide of interest is increased when an encrypted RNA encoding the polypeptide of interest is contacted by a translation activator of the encrypted RNA. As used herein, an encrypted RNA is said to be “activated” by contact with a translation activator.

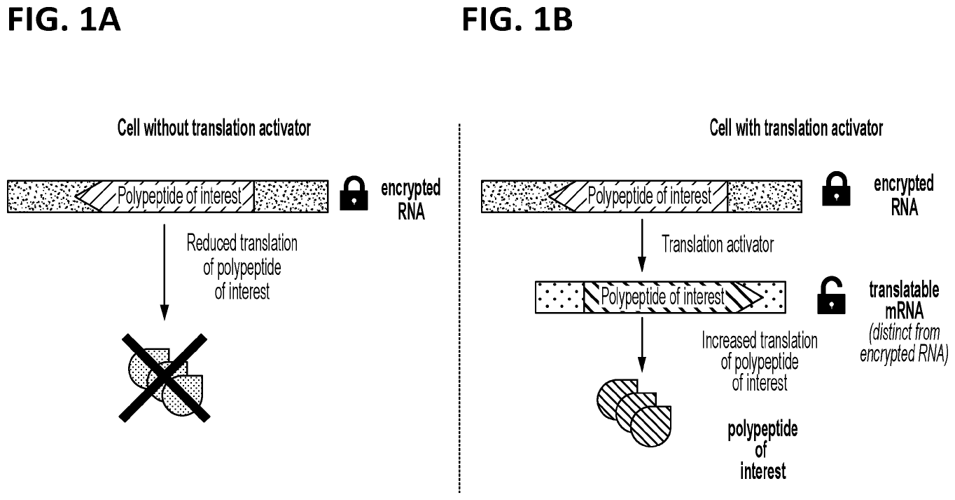

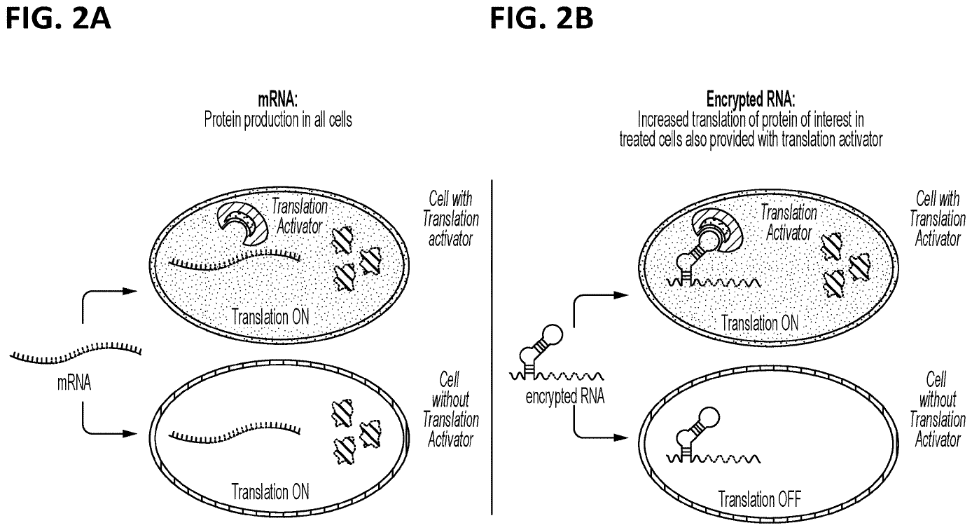

As shown in FIGS. 1A-B and FIGS. 2A-2B, in some embodiments, contact between an encrypted RNA and a translation activator increases translation of the polypeptide of interest.

As used herein, an “encrypted protein” or an “encrypted polypeptide” is a polypeptide of interest encoded by an encrypted RNA.

As used herein, a “therapeutic encrypted RNA” is an encrypted RNA wherein the coding region encodes a therapeutic polypeptide. As used herein, “SHIELD” or “SHIELD RNA” or “SHIELD encrypted RNA” have the same meanings as “therapeutic encrypted RNA”.

As used herein, a “DNA-encoded encrypted RNA” is a DNA sequence that encodes an encrypted RNA cassette.

As used herein, an “encrypted nucleic acid” means an encrypted RNA or a DNA-encoded encrypted RNA.

As used herein, “antisense encrypted RNA” means that the coding sequence, which encodes the polypeptide of interest within the coding region, is positioned in an antisense orientation with respect to the encrypted RNA sequence. As used herein, “negative-sense encrypted RNA” and “(−)-sense encrypted RNA” are equivalent to “antisense encrypted RNA”.

As used herein, “sense encrypted RNA” means that the coding sequence, which encodes the polypeptide of interest within the coding region, is positioned in a sense orientation with respect to the encrypted RNA sequence. As used herein, “positive-sense encrypted RNA” and “(+)-sense encrypted RNA” are equivalent to “sense encrypted RNA”.

As used herein, an “influenza encrypted RNA” is an encrypted RNA with a target-specific translation activator comprising an influenza virus polypeptide. For clarity, an encrypted RNA with a target-specific translation activator comprising an influenza virus polypeptide means the encrypted RNA is activated by an influenza virus polypeptide (e.g., an influenza virus polymerase).

As used herein, an “influenza A encrypted RNA” is an encrypted RNA with a target-specific translation activator comprising an influenza A virus polypeptide. For clarity this means the influenza A encrypted RNA is activated by an influenza A virus polypeptide (e.g., an influenza A virus polymerase).

As used herein, an “influenza B encrypted RNA” is an encrypted RNA activated by an influenza B virus polypeptide (e.g., an influenza B virus polymerase). As used herein, a “therapeutic influenza encrypted RNA” or an “influenza SHIELD” is an influenza encrypted RNA that is a therapeutic encrypted RNA.

As used herein, an “influenza antisense encrypted RNA” or an “influenza negative-sense encrypted RNA” or an “influenza (−)-sense encrypted RNA” is an influenza encrypted RNA that is an antisense encrypted RNA.

As used herein, an “influenza sense encrypted RNA” or an “influenza positive-sense encrypted RNA” or an “influenza (+)-sense encrypted RNA” is an influenza encrypted RNA that is a sense encrypted RNA.

As used herein, a “sarbecovirus encrypted RNA” is an encrypted RNA activated by a sarbecovirus polypeptide (e.g., a sarbecovirus virus polymerase) v.

As used herein, a “therapeutic sarbecovirus encrypted RNA” or a “sarbecovirus SHIELD” is a sarbecovirus encrypted RNA that is a therapeutic encrypted RNA.

As used herein, a “sarbecovirus antisense encrypted RNA” or a “sarbecovirus negative-sense encrypted RNA” or a “sarbecovirus (−)-sense encrypted RNA” is a sarbecovirus encrypted RNA that is a antisense encrypted RNA.

As used herein, a “sarbecovirus sense encrypted RNA” or a “sarbecovirus positive-sense encrypted RNA” or a “sarbecovirus (+)-sense encrypted RNA” is a sarbecovirus encrypted RNA that is a sense encrypted RNA.

As used herein, “SARS-2” is the SARS-COV-2 virus.

As used herein, an “RSV encrypted RNA” is an encrypted RNA activated by a respiratory syncytial virus (RSV) polypeptide (e.g., an RSV polymerase).

As used herein, a “therapeutic RSV encrypted RNA” or an “RSV SHIELD” is an RSV encrypted RNA that is a therapeutic encrypted RNA.

As used herein, an “RSV antisense encrypted RNA” or an “RSV negative-sense encrypted RNA” or an “RSV (−)-sense encrypted RNA” is an RSV encrypted RNA that is an antisense encrypted RNA.

As used herein, an “RSV sense encrypted RNA” or an “RSV positive-sense encrypted RNA” or an “RSV (+)-sense encrypted RNA” is an RSV encrypted RNA that is a sense encrypted RNA.

As used herein, a carrier or polymeric carrier is typically a compound that facilitates transport or complexation of another compound (cargo). A polymeric carrier is typically a carrier that is formed of a polymer. A carrier may be associated with its cargo by covalent or non-covalent interaction. A carrier may transport nucleic acids, e.g. RNA or DNA, to the target cells and/or may facilitate uptake of nucleic acids into the target cells. The carrier may, for some embodiments, be a cationic component.

The term “cationic component” typically refers to a charged molecule, which is positively charged (cation) at a pH value typically from 1 to 9. Accordingly, a cationic component may be any positively charged compound or polymer, such as a cationic peptide or protein or lipid, which is positively charged under physiological conditions, such as those that occur in vivo. A “cationic peptide or protein” may contain at least one positively charged amino acid, or more than one positively charged amino acid, e.g. selected from Arg, His, Lys or Asn. Accordingly, “polycationic” components are also within the scope exhibiting more than one positive charge under the conditions given.

The term “subject” refers to an animal, for example a human, to whom treatment, including prophylactic treatment, with methods, polynucleotides (including encrypted RNAs and DNA encoding encrypted RNAs) and compositions described herein, is provided. For treatment of those conditions or disease states which are specific to a specific animal such as a human subject, the term “subject” refers to that specific animal. Cells, tissues, and progeny of said cells or tissues obtained in vivo or cultured ex vivo or in vitro are also included. In addition to humans, subjects include cows, pigs, sheep, horses, deer, other rumenants, rodents, fish, and fowl (e.g., chickens and ducks).

The term “tissue” refers to a group or layer of similarly specialized cells which together perform certain special functions.

“Gene therapy” may typically be understood to mean a treatment of a patient's body or isolated elements of a patient's body, for example isolated tissues/cells, by nucleic acids encoding a peptide or protein. It typically may comprise at least one of the steps of a) administration of a nucleic acid directly to the patient—by any applicable administration route—or in vitro to isolated cells/tissues of the patient, which results in transfection of the patient's cells either in vivo/ex vivo or in vitro; b) transcription and/or translation of the introduced nucleic acid molecule; and optionally c) re-administration of isolated, transfected cells to the patient, if the nucleic acid has not been administered directly to the patient. The term “gene therapy” as used herein typically encompasses treatment as well as prevention or prophylaxis of a disease or disorder.

As used herein, it is understood that RNA polynucleotides are comprised of ribonucleotide monomers and that DNA polynucleotides are comprised of deoxyribonucleotide monomers. As ribonucleotides are nucleotides and deoxyribonucleotides are nucleotides, the leading “ribo” or “deoxyribo” can be omitted when the meaning is clear. As an example, “an RNA polynucleotide comprised of nucleotides” has the same meaning as “an RNA polynucleotide comprised of ribonucleotides”. Likewise, “a DNA polynucleotide comprised of nucleotides” has the same meaning as “a DNA polynucleotide comprised of deoxyribonucleotides”

“RNA” is the usual abbreviation for ribonucleic acid. It is a nucleic acid molecule or polynucleotide, i.e. a polymer consisting of ribonucleotides (nucleotides). These nucleotides are usually adenosine monophosphate (AMP), cytidine monophosphate (CMP), guanosine-monophosphate (GMP), and uridine monophosphate (UMP) monomers, which are connected to each other along a so-called backbone or phosphodiester backbone. When the meaning is clear, RNA polynucleotides may be said to be comprised of their nucleotide triphosphates, e.g., adenine triphosphate (ATP), cytidine triphosphate (CTP), guanosine triphosphate (GTP), or uridine triphosphate (UTP), indicating that an RNA polynucleotide was synthesized or transcribed using nucleotide triphosphate monomers to form a usual RNA polynucleotide.

The backbone is formed by phosphodiester bonds between the sugar, i.e., ribose, of a first monomer and a phosphate moiety of a second, adjacent monomer. The specific succession of the monomers is called the RNA sequence. Usually, RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA, which has to be processed into so-called messenger RNA, usually abbreviated as mRNA. Processing of the premature RNA, e.g., in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino-acid sequence of a particular polypeptide or protein. Typically, a mature mRNA comprises a 5′-UTR, an open reading frame, and a 3′-UTR. Aside from messenger RNA, several types of RNA exist, which may be involved in regulation of transcription or translation.

As used herein, “nucleoside-modified” means that an RNA polynucleotide is comprised of at least one nucleotide that is not AMP, CMP, GMP, or UMP.

As used herein, the terms “nucleoside-modified RNA” or “nucleoside-modified encrypted RNA” or “nucleoside-modified therapeutic encrypted RNA” or “nucleoside-modified SHIELD” or “nucleoside-modified mRNA” refer to RNA molecules containing one, two, or more than two nucleoside modifications compared to adenosine (A) ((2R,3R,4S,5R)-2-(6-amino-9H-purin-9-yl)-5-(hydroxymethyl)oxolane-3,4-diol), guanosine (G) (2-Amino-9-[3,4-dihydroxy-5-(hydroxymethyl)oxolan-2-yl]-3H-purin-6-one), cytidine (C) (4-amino-1-[3,4-dihydroxy-5-(hydroxymethyl)tetrahydrofuran-2-yl]pyrimidin-2-one), or uridine (U) (1-[(3R,4S,5R)-3,4-dihydroxy-5-(hydroxymethyl)oxolan-2-yl]pyrimidine-2,4-dione), or compared to AMP, GMP, CMP, or UMP, in RNA molecules, or a portion thereof. Non-limiting examples of nucleoside modifications are provided elsewhere in herein. Where the nucleotide sequence of a particular claimed RNA is otherwise identical to the sequence of a naturally-existing RNA molecule, the nucleoside-modified RNA is understood to be an RNA molecule with at least one modification different from those existing in the naturally occurring counterpart. The difference can be either in the chemical change to the nucleoside/nucleotide.

In some embodiments, between about 30%-100% of UMP nucleotides within a nucleoside-modified RNA are replaced with a modified nucleoside. In some embodiments, about 30%, 35%, 40%, 45%, 50%, 55%, 60%, 65%, 70%, 75%, 80%, 85%, 90%, 95%, 99% or about 100% of UMP nucleotides within a nucleoside-modified RNA are replaced with a modified nucleoside. In some embodiments, between about 30%-100% of CMP nucleotides within a nucleoside-modified RNA are replaced with a modified nucleoside. In some embodiments, between about 30%-100% of CMP nucleotides within a nucleoside-modified RNA are replaced wth a modified nucleoside. In some embodiments, about 30%, 35%, 40%, 45%, 50%, 55%, 60%, 65%, 70%, 75%, 80%, 85%, 90%, 95%, 99% or about 100% of CMP nucleotides within a nucleoside-modified RNA are replaced with a modified nucleoside.

In some embodiments, between about 1%-30% of AMP nucleotides within a nucleoside-modified RNA are replaced with a modified nucleoside. In some embodiments, about 1%, 2%, 3, 4%, 5%, 10%, 15%, 20%, 25%, or about 30% of AMP nucleotides within a nucleoside-modified RNA are replaced with a modified nucleoside.

In some embodiments, a nucleoside-modified RNA includes at least one UMP that is modified to form N1-methyl-pseudo-UMP (N1-methylpseudouridine, N1m-pU). In some embodiments, a nucleoside-modified RNA includes at least one UMP that is modified to form pseudo-UMP (pseudouridine, pU). In a nucleoside-modified RNA, not all nucleosides need to be modified. In some embodiments, between about 10% and about 100% of UMP nucleotides within a nucleoside-modified RNA are replaced with pseudo-UMP or with N1-methyl-pseudo-UMP. In some embodiments, about 10%, 15%, 20%, 25%, 30%, 35%, 40%, 45%, 50%, 55%, 60% or about 70% of UMP nucleotides within a nucleoside-modified RNA are replaced with pseudouridine or N1-methyl-pseudo-UMP. In some embodiments, between about 10% and 35% of UMP nucleotides within a nucleoside-modified RNA are replaced with pseudo-UMP or N1-methyl-pseudo-UMP. In some embodiments, between about 10%, 15%, 20%, 25%, 30%, or about 35% of UMP nucleotides within a nucleoside-modified RNA are replaced with pseudo-UMP or N1-methyl-pseudo-UMP. In some embodiments, about 100% of UMP nucleotides within a nucleoside-modified RNA are replaced with pseudo-UMP or N1-methyl-pseudo-UMP. In some embodiments, about 70% of UMP nucleotides within a nucleoside-modified RNA are replaced with pseudo-UMP nucleotides. In some embodiments, about 100% of UMP nucleotides within a nucleoside-modified RNA are replaced with pseudo-UMP nucleotides. In some embodiments, a nucleoside-modified RNA includes at least one AMP that is modified to form N6-methyl-AMP. In some embodiments, a nucleoside-modified RNA includes at least one CMP that is modified to form 5-methyl-CMP. In some embodiments, a nucleoside-modified RNA includes at least one UMP that is modified to form 5-methoxy-UMP (moU). In some embodiments, the RNA does not comprise any nucleoside-modifications (unmodified RNA).

As used herein, “capped RNA” or “5′-capped RNA” refers to RNA molecules incorporating a Cap structure at their 5′ end. Cap structures are present on the 5′-end of many mRNAs in eukaryotic organisms as well as on the viral RNA of some viruses.

Naturally occurring Cap structures typically comprise a riboguanosine residue that is methylated at position N7 of the guanine base. This N7-methylguanosine (m7G) is linked via a 5′- to 5′-triphosphate chain at the 5′-end of the mRNA molecule. 5′-capping of RNA can facilitate resistance to degradation by exonucleases and facilitates transport of mRNAs from the nucleus to the cytoplasm. Naturally-occurring examples of Cap structures include Cap 0, Cap 1, and Cap 2. When the only capping modification is an N7-methylguanosine linked to the terminal nucleotide of the RNA via a 5′-to-5′-triphosphate linkage, the structure is referred to as Cap 0. When the RNA additionally incorporates a 2′-O-methylation of only the first nucleoside 5′ of Cap 0 (i.e., the penultimate nucleoside of the RNA, inclusive of m7G), the structure is referred to as Cap 1. When the RNA additionally incorporates 2′-O-methylation of the first two nucleosides 5′ of Cap 0 (i.e., both the penultimate and the antepenultimate nucleoside, inclusive of m7G), the structure is referred to as Cap 2.

Cap 0 (3′-O-Me) is Cap 0 in which the 3′-OH (i.e., 3′ hydroxyl group) of the 5′ N7-methylguanosine (m7G) cap of Cap 0 is replaced by —OCH₃ (i.e., 3′ methoxy group). Similarly, Cap 1 (3′-O-Me) and Cap 2 (3′-O-Me) are Cap 1 and Cap 2 structures which include a 3′-O-methylation of the 5′ N7-methylguanosine (m7G) cap relative to the respective Cap 1 or Cap 2.

In some embodiments, a capped RNA contains a 5′-Cap structure that is a Cap 0, Cap 0 (3′-O-Me), Cap 1, Cap 1 (3′-O-Me), Cap 2, Cap 2 (3′-O-Me), Anti-Reverse Cap Analog (ARCA), inosine, N1-methyl-guanosine, 2′-fluoro-guanosine, 7-deaza-guanosine, 8-oxo-guanosine, 2-amino-guanosine, locked nucleic acid guanosine (LNA-guanosine), or 2-azido-guanosine structure. All of these represent nucleoside-modified RNA molecules.

As used herein, “uncapped RNA” or “noncapped RNA” refers to RNA molecules that lack a 5′-Cap structure.

As used herein, “5′-phosphorylation” refers to the number of consecutive phosphate molecules attached to the 5′-end of uncapped RNA. RNA molecules which are “triphosphorylated” or “5′-triphosphorylated” are uncapped and have a 5′-terminal triphosphate (3 phosphates). RNA molecules which are “5′-diphosphorylated” or “5′-biphosphorylated” are uncapped and have a 5′-terminal diphosphate (2 phosphates). RNA molecules which are “monophosphorylated” or “5′-monophosphorylated” are uncapped and have a 5′-terminal monophosphate or 5′-terminal phosphate (1 phosphate). RNA molecules which are “nonphosphorylated” or “5′-nonphosphorylated” have no 5′ terminal phosphate (0 phosphates).

A “polymerase” generally refers to a molecular entity capable of catalyzing the synthesis of a polymeric molecule from monomeric building blocks. An “RNA polymerase” is a molecular entity capable of catalyzing the synthesis of an RNA molecule from ribonucleotide building blocks. A “DNA polymerase” is a molecular entity capable of catalyzing the synthesis of a DNA molecule from deoxyribonucleotide building blocks. For the case of DNA polymerases or RNA polymerases, the molecular entity is typically a protein or an assembly or complex of multiple proteins. Typically, a DNA polymerase synthesizes a DNA molecule based on a template nucleic acid, which is typically a DNA molecule. Some DNA polymerases are RNA-dependent DNA polymerases and synthesize DNA molecules based on template nucleic acids. Some RNA-dependent DNA polymerases are termed “reverse transcriptases”. Typically, an RNA polymerase synthesizes an RNA molecule based on a template nucleic acid, which is either a DNA molecule (in that case the RNA polymerase is a DNA-dependent RNA polymerase, DdRP), or an RNA molecule (in that case the RNA polymerase is an RNA-dependent RNA polymerase, RdRP).

“RNA dependent RNA polymerases” or “RdRPs” are multi-domain (a and B) proteins that catalyze RNA-template dependent formation of phosphodiester bonds between ribonucleotides in the presence of divalent metal ions. The initiation of synthesis occurs at the 3′-end of the template in a primer-dependent or independent manner and proceeds on the synthesized strand in the 5′->3′ direction. The average length of the core RdRP domain is less than 500 amino acids and is folded into three subdomains. The active sites of RdRPs from different RNA viruses are conserved and show resemblances to those of other enzymes such as reverse transcriptases and DNA polymerases indicating their similar role in nucleotidyl transfer reactions.

Some viral polymerases possess additional domains such as methyltransferase or endonuclease domain to carry out functions associated with RNA synthesis. The polymerase domain may also interact with other host factors for efficient polymerization and to discriminate activities such as genome replication and mRNA transcription. The host factors include translation factors, protein chaperones, RNA-modifying enzymes, or other cellular proteins. These together with the RdRPs, constitute the viral replication complexes (VRCs). The VRCs differ in their composition, subcellular location, and interaction with the viral RNA templates.

As defined herein, a “ribozyme” is a catalytic macromolecular complex comprising an RNA with catalytic activity. Examples of ribozymes include, without limitation, an RNA molecule with a self-splicing intron sequence, an RNA molecule comprised of the Hepatitis Delta Virus antigenomic ribozyme, an RNA molecule comprised of a “Hammerhead” ribozyme, and a two-component ribonucleoprotein system comprising a guide RNA complexed with a Cas protein (“CRISPR-Cas”). RNA molecules comprising a ribozyme with nuclease activity may cleave within the molecule in which they are embedded or may cleave RNA outside of the molecule in which they are embedded.

As used herein, “sequence identity”, is used to mean a relationship between two or more protein (polypeptide) sequences or two or more nucleic acid (polynucleotide) sequences, as determined by comparing the sequences. Two or more sequences are identical if they exhibit the same length and order of nucleotides or amino acids. Calculation of the percent identity (or % identity) of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, 96%, 97%, 98%, 99%, or 100% of the length of a reference sequence. The nucleotides at corresponding nucleotide positions are then compared. When a position in the first sequence is occupied by the same nucleotide or amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using an algorithm. For example, the percent identity between two nucleotide sequences or two polypeptide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference.

Polynucleotide or polypeptide sequences can be compared by performing a sequence alignment, which may be gapped or ungapped. In an ungapped alignment, two or more sequences are compared as “contiguous” sequences, i.e., one sequence is aligned with the other sequence and each nucleotide or amino acid in one sequence is directly compared with the corresponding nucleotide or amino acid in the other sequence, one residue at a time. In an ungapped alignment, in an otherwise identical pair of sequences, one insertion or deletion may cause the other nucleotide or amino acid residues to be put out of alignment, thus resulting in a potentially non-optimal global alignment. In a gapped alignment, sequences are compared “non-contiguously”, and insertions and deletions (collectively “gaps”) may be inserted to optimally align the sequences.

As used herein, “sequence similarity”, is used in a like manner to “sequence identity”, but captures aspects of relatedness between two sequences, such as functional or phenotypic relatedness, that may not be fully explained by methods to determine sequence identity.

Methods to determine identity and similarity are codified in publicly available algorithms or software, including: BLAST, FASTA, T-COFFEE, and M-COFFEE. In some methods, a scaled similarity score matrix or equivalent can be used to assign a score to each pairwise comparison based on chemical similarity or evolutionary distance. An example of such a matrix commonly used is the BLOSUM62 matrix—the default matrix for the Basic Local Alignment Search Tool (BLAST) suite of programs. There are also alternative computational methods used to determine identity or similarity (e.g., INFERNAL or R-COFFEE) which also consider aspects of sequence relatedness (e.g., covariance models, secondary structure, or tertiary structure) in addition to the primary sequence (Eddy & Durbin, Nucleic Acids Research (1994); DOI: 10.1093/nar/22.11.2079) (Rivas et al., Bioinformatics (2020); DOI: 10.1093/bioinformatics/btaa080) (Nawrocki & Eddy; Bioinformatics (2013); DOI: 10.1093/bioinformatics/btt509).

In some embodiments, encrypted RNAs with different template regions can be activated by the same translation activator. Therefore, template regions may share a common structure and function although their primary nucleotide sequences differ: i.e., template regions of the same translation activator may be non-identical but similar sequences.

In some embodiments, an encrypted RNA with a variant template region will have the same or similar activation in the presence or absence of a translation activator as an encrypted RNA with a reference template region. Alternatively, an encrypted RNA with the variant template region may have altered activation (e.g., increased or decreased) relative to the encrypted RNA with a reference template region. Generally, the variant template region will have similarity or identity to the reference template region of at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% but less than 100% sequence identity to that particular reference polynucleotide as determined by sequence alignment programs and parameters described herein and known to those skilled in the art. As used herein, a “variant” of a nucleotide sequence is one that has less than 100% sequence identity to a reference nucleotide sequence due to a substitution of at least one nucleotide for another, an addition (insertion) of one or more nucleotides, and/or a deletion of one or more nucleotides relative to a reference sequence. As used herein, a “variant” of a polypeptide sequence is one that has less than 100% sequence identity to a reference polypeptide sequence due to a substitution of at least one amino acid for another, an addition (insertion) of one or more amino acids, and/or a deletion of one or more amino acids relative to a reference sequence.

In some embodiments, two different translation activators can activate the same encrypted RNA. Therefore, translation activators may share a common structure and function although their primary polypeptide sequences differ.

In some embodiments, a translation activator comprising a variant polypeptide (e.g., a variant polymerase) will similarly activate an encrypted RNA as a translation activator comprising a reference polypeptide. Alternatively, a translation activator comprising a variant polypeptide may have altered activation of an encrypted RNA (e.g., increased or decreased) relative to the a translation activator comprising a reference polypeptide. Generally, the variant polypeptide will have similarity or identity to the reference template region of at least about 30%, 35%, 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polypeptide as determined by sequence alignment programs and parameters described herein and known to those skilled in the art.

A “stabilized nucleic acid molecule” is a nucleic acid molecule, typically a DNA or RNA molecule, that is modified such that it is more stable to disintegration or degradation, e.g., by environmental factors or enzymatic digest such as by exo- or endonuclease degradation, than the nucleic acid molecule without the modification. In some embodiments, a stabilized nucleic acid molecule is stabilized against degradation in a cell, such as a prokaryotic or eukaryotic cell. In some further embodiments, a stabilized nucleic acid molecule is stabilized against degradation in a mammalian cell, such as a human cell. The stabilization effect may also be exerted outside of cells, e.g., in a buffer solution etc., for example, in a manufacturing process for a pharmaceutical composition comprising the stabilized nucleic acid molecule.

The term “transfection” refers to the introduction of nucleic acid molecules, such as DNA or RNA (e.g., mRNA) molecules, into cells, such as eukaryotic cells. In the context of the present disclosure, the term “transfection” encompasses any method known to the skilled person for introducing nucleic acid molecules into cells, such as into mammalian cells. Such methods encompass, for example, electroporation, lipofection, e.g., based on cationic lipids or liposomes, calcium phosphate precipitation, nanoparticle based transfection, virus based transfection, or transfection based on cationic polymers, such as DEAE-dextran or polyethylenimine, etc.

The term “vector” refers to a nucleic acid molecule. A vector in the context of the present disclosure is suitable for incorporating or harboring a desired nucleic acid sequence, such as a nucleic acid sequence comprising an open reading frame. Such vectors may be storage vectors, expression vectors, cloning vectors, transfer vectors, etc. A storage vector is a vector, which allows the convenient storage of a nucleic acid molecule, for example, of an mRNA molecule. Thus, the vector may comprise a sequence corresponding, e.g., to a desired mRNA sequence or a part thereof, such as a sequence corresponding to the coding sequence and the 3′-UTR of an mRNA. An expression vector may be used for production of expression products, such as: RNA, encrypted RNA, mRNA, peptides, polypeptides, or proteins. An expression vector may comprise sequences needed for transcription of a sequence stretch of the vector, such as a promoter sequence, e.g., an RNA polymerase promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector, which is suitable for transferring nucleic acid molecules into cells or organisms, for example, viral vectors. In some embodiments, the viral vector is a lentiviral vector. A vector in the context of the present disclosure may be, e.g., an RNA vector or a DNA vector. In some embodiments, the vector is a DNA molecule. In some embodiments, the vector comprises a cloning site, a selection marker, such as an antibiotic resistance factor, and/or a sequence suitable for multiplication of the vector, such as an origin of replication. In some embodiments, the vector is a plasmid vector, also referred to as a plasmid.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, a “lentiviral vector” is a viral vector derived from a lentivirus.

A “vehicle” is typically understood to be a material that is suitable for storing, transporting, or administering a compound, such as a pharmaceutically active compound. For example, it may be a physiologically acceptable liquid, which is suitable for storing, transporting, or administering a pharmaceutically active compound.

### Encrypted RNAs

Aspects of the present disclosure provide encrypted RNAs, including DNA encoding encrypted RNAs, comprising

In some embodiments, an encrypted RNA is a single stranded RNA (ssRNA)s.

In some embodiments, an encrypted RNA is a capped ssRNA.

In some embodiments, an encrypted RNA is an uncapped ssRNA.

In some embodiments, an encrypted RNA is a 5′-triphophosphorylated uncapped ssRNA.

In some embodiments, an encrypted RNA is a 5′-diphosphorylated uncapped SSRNA.

In some embodiments, an encrypted RNA is a 5′-monophosphorylated uncapped ssRNA.

In some embodiments, an encrypted RNA is a 5′-nonphosphorylated uncapped ssRNA.

In some embodiments, an encrypted RNA is an uncapped ssRNA with four or more 5′-terminal phosphates (tetraphosphates, pentaphosphates).

In some embodiments, an encrypted RNA is a stabilized nucleic acid molecule.

In some embodiments, an encrypted RNA is a circular RNA.

In some embodiments, the template region of an encrypted RNA or the translation activator is not derived from an alphavirus genome.

In some embodiments, an encrypted RNA is delivered to cells and is translated at low levels until it contacts a polymerase encoded by an infectious virus that then results in increased translation of the encrypted RNA.

In some embodiments, an encrypted RNA is delivered to cells together with a target-specific translation activator.

In some embodiments, DNA is used to encode an encrypted RNA.

In some embodiments, an encrypted nucleic acid is a stabilized nucleic acid molecule.

In some embodiments, a DNA sequence which flanks an encrypted RNA within a DNA-encoded encrypted RNA cassette can have a desirable effect on the production of the encrypted RNA by inducing one or more outcomes, including: altering the level (abundance) of encrypted RNA produced, altering the average molecular structure of the encrypted RNA, or altering the rate at which encrypted RNA is produced from the DNA template.

In some embodiments, a DNA-encoded encrypted RNA cassette can be repurposed to produce other RNA species by substitution of the encrypted RNA sequence with an alternative, non-encrypted RNA sequence encoding an RNA. In some embodiments, a DNA-encoded encrypted RNA cassette can be repurposed to encode non-encrypted RNA sequences which encode viral genetic elements. In some embodiments, a DNA-encoded encrypted RNA cassette can be repurposed to encode non-encrypted RNA sequences which are antisense to a targeted sequence. In some embodiments, a DNA-encoded encrypted RNA cassette can be repurposed to encode non-encrypted RNA sequences which encode guide RNAs for a CRISPR-Cas system. In some embodiments, a DNA-encoded encrypted RNA cassette can be repurposed to encode non-encrypted RNA sequences which encode mRNA sequences.

In some embodiments, the DNA encoding an encrypted RNA is delivered to cells in a viral vector.

In some embodiments, the DNA encoding an encrypted RNA is delivered to cells in a plasmid.

In some embodiments, an encrypted RNA or the DNA encoding the encrypted RNA encodes a therapeutic protein, for example an immunomodulatory protein.

In some embodiments, an encrypted RNA or a DNA encoding an encrypted RNA encodes more than one polypeptide of interest. Strategies for encoding multiple polypeptides are well-known to practioners skilled in the art (see, for example, Liu et al., Scientific Reports (2017) DOI: 10.1038/s41598-017-02460-2). Such strategies include use of multiple promoters, fusion proteins, proteolytic cleavage sites within polypeptides, internal ribosome entry sites, and “ribosomal skipping” 2A peptides, In some embodiments, more than one polypeptide of interest is encoded in a coding sequence that is translated as two or more polypeptides, through the action of one or more “2A” like sequences present in a coding sequence (e.g., the 2A sequence from porcine teschovirus-1, the 2A sequence from foot-and-mouth disease virus, the 2A sequence from equine rhinitis A virus, or the 2A sequence from thosea asigna virus). See, e.g., Liu et al. Scientific Reports (2017) 7:2193.

In some embodiments, a target-specific translation activator comprises a polymerase. In some embodiments, a translation activator comprises an RNA-Dependent RNA Polymerase (“RdRP”) or an RNA-Dependent DNA Polymerase (“RdDP”). In some further embodiments, a translation activator comprises additional polypeptides that facilitate mRNA synthesis. In some further embodiments, the additional polypeptides include: matrix proteins, nucleoproteins, or non-structural proteins.

RNA viruses are quite diverse in virus particle and genome structure and in virus entry and assembly mechanisms. However, they do share fundamental features in their genome replication and transcription, often using a virally encoded RdRP to carry out the biosynthesis of an RNA product directed by an RNA template. Although the genome replication machinery often requires the participation of other factors, typically at the initiation phase of synthesis, the RdRP governs the elongation phase of synthesis that includes thousands of efficient nucleotide addition cycles (NACs). Viral RdRPs vary greatly in size and structural organization, from the ~50-kDa picornavirus 3Dpol, to the ~100-kDa flavivirus NS5 that contains a naturally fused methyltransferase domain, to the ~250-kDa RSV L protein harboring at least three enzymatic domains, to the ~260-kDa three-subunit PA-PB1-PB2 influenza virus replicase complex. On the other hand, all RdRPs share a 50- to 70-kDa polymerase core that forms a unique encircled right-hand structure with palm, fingers, and thumb domains. Among the seven classic RdRP catalytic motifs, A-E are within the most conserved palm domain, and F and G are located in the fingers; they are all arranged similarly around the active site. The structural conservation of the RdRP polymerase core and the seven motifs form the basis for understanding the common features in viral RdRP catalytic mechanism and for finding intervention strategies targeting these enzymes with possible broad-spectrum potential.

In some embodiments, an encrypted RNA resembles an RNA that is viral in origin, but instead of encoding a polypeptide of interest that is native to the virus (homologous), the encrypted RNA encodes a therapeutic polypeptide of interest that is not native to the virus (heterologous). In this instance, the construct of the encrypted RNA encoding the therapeutic polypeptide of interest is arranged with a flanking L region and a flanking R region and the arrangement is not “native” to the virus.

In some embodiments, an encrypted RNA resembles a viral RNA and possesses sufficient cis-acting sequences to be encapsidated into viral particles. In some further embodiments, encrypted RNA possesses sufficient cis-acting sequences to be encapsidated into viral particles that are infectious and can transmit and deliver encrypted RNA to additional cells via viral infection. In some embodiments, the RNA species produced after an encrypted RNA is contacted by a target-specific translation activator is competent for encapsidation into viral particles. In some further embodiments, the RNA species produced after an encrypted RNA is contacted by a target-specific translation activator possesses sufficient cis-acting sequences to be encapsidated into viral particles that are infectious and can transmit and deliver the produced RNA species to additional cells via viral infection.

The negative-strand RNA viruses of animals are divided into several families and include the agents of well-known diseases such as rabies, mumps, measles, and influenza as well as more emerging pathogens such as Ebola virus. In all of these, the single-stranded RNA in the virus particle is complementary to the mRNA and is therefore the minus strand. These viruses vary in shape and structure but are similar in having an outer envelope derived from the membrane of the host cell where they were assembled. Thus, the RNA in negative-strand RNA viruses is the antisense strand.

After infiltrating the cell, a key mission of a negative-strand RNA virus is to make its RNA double-stranded by synthesizing the corresponding positive RNA strand. Once it becomes double-stranded, it uses both RNA strands as templates. The plus strand (alternatively written as “+strand”) is used as a template to manufacture more negative strands for the next generation of virus particles. The minus strand (alternatively written as “-strand”) is used as a template to manufacture multiple positive strands that act as mRNA molecules. This strategy is not only an effective division of labor but also avoids the problem of translating multiple reading frames on a single incoming virus RNA molecule.

Positive-strand RNA viruses, also known as a sense-strand RNA viruses, are viruses whose genetic information consists of a single strand of RNA that is the positive (or sense) strand which encodes mRNA and protein. Replication in positive-strand RNA viruses proceeds through a negative-strand intermediate. Non-limiting examples of positive-strand RNA viruses include coronaviruses, polio virus, Coxsackie virus, and echovirus.

Some RNA viruses, including all retroviruses and lentiviruses, produce a DNA copy of their RNA genome during an aspect of their natural viral lifecycle. A virally-encoded RdDP or reverse transcriptase is the polymerase which reverse transcribes the viral genomic RNA into a DNA copy which can be subsequently integrated into a host cell chromosome or be retained extrachromosomally as an episome. The term provirus or proviral DNA can be used to describe the DNA copy of a retroviral genome.

Some DNA viruses, such as those of the family Hepadnaviridae (a member of which is Hepatitis B Virus, “HBV”), replicate their DNA viral genome through an RNA intermediate and possess an RdDP or reverse transcriptase to convert the RNA intermediate into DNA template molecule for further genome amplification.

In some embodiments, a sequence within the encrypted RNA is converted to DNA by a translation activator comprised of an RdDP or a reverse transcriptase. The DNA sequence can then further transcribed into mRNA by a translation activator.

Some RNA viruses, such as Hepatitis Delta Virus (HDV), are thought to use the minor RdRP activity of certain host RNA polymerases, including human RNA Polymerase I (human Pol I), human RNA Polymerase II (human Pol II), or human RNA Polymerase III (human Pol III) to transcribe their viral RNA to produce mRNA. These host RNA polymerases are typically thought to be primarily DNA-Dependent RNA Polymerases, but may be able to synthesize RNA from a DNA template or from an RNA template.

In some embodiments, the translation activatoe of an encrypted RNA is comprised of a polymerase that can synthesize RNA from a DNA template or an RNA template.

In some embodiments, the translation activator of an encrypted RNA is comprised of viral RdRPs. Without wishing to be bound to any particular theory, it is thought that activation of an encrypted RNA into mRNA occurs because the encrypted RNA contains virus-derived sequences that can bind to viral RdRP complexes. In some embodiments, activation of an encrypted RNA occurs because the encrypted RNA contains virus-derived sequences that can bind to viral RdDP complexes. In some embodiments, the polypeptide of interest of a therapeutic encrypted RNA is translated at reduced levels by host cell ribosomal machinery in the absence of viral infection, with increased therapeutic protein production upon viral infection.

In some embodiments, an encrypted RNA can resemble viral replication intermediates that a virus synthesizes into mRNA to replicate its genome. In other words, both the encrypted RNA and the reverse complement of the encrypted RNA can be activated by a translation activator. In some embodiments, when virus infection ends, translation of the polypeptide of interest of an encrypted RNA substantially ends due to the short half-life of produced mRNA and of the polypeptide of interest and an inability to substantially produce new mRNA in the absence of the translation activator.

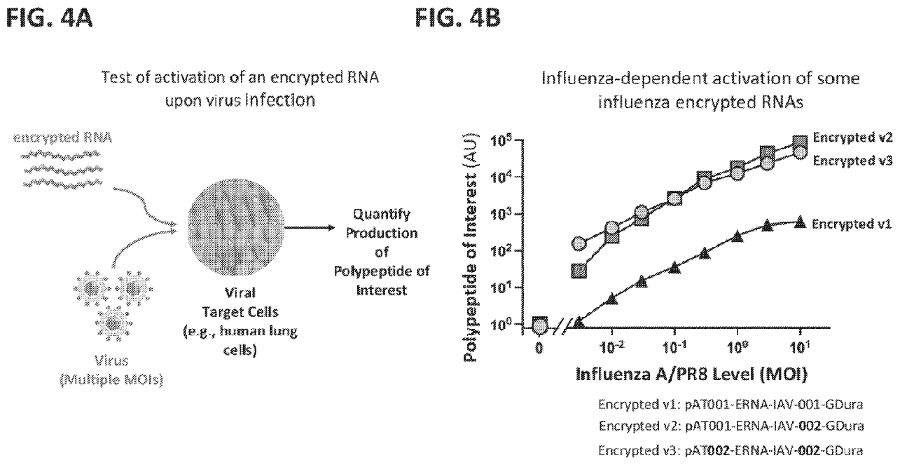

In some embodiments, encrypted RNAs do not contain internal ribosome entry site (IRES) sequences. In some embodiments, encrypted RNAs are engineered to lack features that are important for efficient protein translation by host cell ribosomes, such as: a 5′-Cap or a 3′ poly(A) tail. In some embodiments, translation of a polypeptide of interest encoded by an encrypted RNA can be increased by at least 10-fold, 100-fold, 1000-fold, 10⁴-fold, 10⁵-fold or more in the presence of virus infection (see, for example, FIG. 4).

In some embodiments, the virus is selected from the group consisting of viruses in the orders of Amarillovirales, Articulavirales, Blubervirales, Bunyavirales, Hepelivirales, Martellivirales, Mononegavirales, Nidovirales, and Picornavirales. In some embodiments, the virus is selected from the group consisting of viruses in the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, Rhabdoviridae, and Togaviridae. In some embodiments, the virus is selected from the group consisting of Alphacoronavirus 229E, Alphacoronavirus NL63, Alphacoronavirus WA2028, Avian metapneumovirus (AMPV), Betacoronavirus HKU1, Betacoronavirus HKU15, Betacoronavirus HKU33, Betacoronavirus OC43, Chikungunya virus, Crimean-Congo Hemorrhagic Fever Virus, Dengue Virus, Eastern Equine Encephalitis Virus (EEEV), Enterovirus D68 (EV-D68), Foot and Mouth Disease Virus, Hanta Virus, Hendra Virus, Hepatitis B Virus, Hepatitis C Virus, HMPV, Human Parainfluenzavirus 1 (HPIV1), Human Parainfluenzavirus 3 (HPIV3), Infectious Salmon Anemia Virus, Influenza A Virus, Influenza B Virus, Lassa Virus, Marburg Virus, Middle East Respiratory Syndrome Coronavirus (MERS-CoV), Newcastle Disease Virus (NDV), Nipah Virus, Norwalk Virus, Rabies Virus, Respiratory Syncytial Virus, Reston Ebola virus, Rhinovirus, Rift Valley Fever Virus, Rubella virus, SARS-COV-1, SARS-COV-2, Sudan Ebola virus, Venezuelan Equine Encephalitis Virus (VEEV), Vesicular Stomatitis Virus, Western Equine Encephalitis Virus (WEEV), Yellow Fever Virus, Zaire Ebola virus, and Zika Virus.

In some embodiments, the virus does not belong to any one of the orders select from Amarillovirales, Articulavirales, Blubervirales, Bunyavirales, Hepelivirales, Martellivirales, Mononegavirales, Nidovirales, and Picornavirales. In some embodiments, the virus does not belong to any one of the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, Rhabdoviridae, and Togaviridae.

In some embodiments, the virus is not an Alphacoronavirus. In some embodiments, the virus is not a metapneumovirus. In some embodiments, the virus is not a Betacoronavirus. In some embodiments, the virus is not a Chikungunya virus. In some embodiments, the virus is not Crimean-Congo Hemorrhagic Fever Virus. In some embodiments, the virus is not Dengue Virus. In some embodiments, the virus is not Eastern Equine Encephalitis Virus (EEEV). In some embodiments, the virus is not Enterovirus D68 (EV-D68). In some embodiments, the virus is not Foot and Mouth Disease Virus. In some embodiments, the virus is not Hanta Virus. In some embodiments, the virus is not Hendra Virus. In some embodiments, the virus is not Hepatitis B Virus. In some embodiments, the virus is not Hepatitis C Virus. In some embodiments, the virus is not HMPV. In some embodiments, the virus is not Parainfluenzavirus 1 (HPIV1). In some embodiments, the virus is not Infectious Salmon Anemia Virus. In some embodiments, the virus is not Influenza A Virus. In some embodiments, the virus is not Influenza B Virus. In some embodiments, the virus is not Lassa Virus. In some embodiments, the virus is not Marburg Virus. In some embodiments, the virus is not Middle East Respiratory Syndrome Coronavirus (MERS-CoV). In some embodiments, the virus is not Newcastle Disease Virus (NDV). In some embodiments, the virus is not Nipah Virus. In some embodiments, the virus is not Norwalk Virus. In some embodiments, the virus is not Rabies Virus. In some embodiments, the virus is not Respiratory Syncytial Virus. In some embodiments, the virus is not Ebola virus. In some embodiments, the virus is not Rhinovirus. In some embodiments, the virus is not Rift Valley Fever Virus. In some embodiments, the virus is not Rubella virus. In some embodiments, the virus is not SARS-COV-1. In some embodiments, the virus is not SARS-COV-2. In some embodiments, the virus is not Sudan Ebola virus. In some embodiments, the virus is not Venezuelan Equine Encephalitis Virus (VEEV). In some embodiments, the virus is not Vesicular Stomatitis Virus. In some embodiments, the virus is not Western Equine Encephalitis Virus (WEEV). In some embodiments, the virus is not Yellow Fever Virus. In some embodiments, the virus is not Zaire Ebola virus. In some embodiments, the virus is not Zika Virus.

Also within the scope of the present disclosure are methods of producing any of the encrypted RNAs described herein. In some embodiments, encrypted RNA is produced outside a cell via in vitro transcription (IVT) using an RNA polymerase and a DNA template molecule that encodes the encrypted RNA. In some embodiments, the encrypted RNA is prepared via IVT as a precursor molecule that is subsequently processed to yield the encrypted RNA. Methods of using in vitro transcription to produce a RNA, including methods that produce RNA having reduced levels or free of immunostimulatory byproducts, are evident to one of ordinary skill in the art. See, e.g., Dousis et al. Nature Biotechnology (2023) 41:560-568.

In some embodiments, the encrypted RNAs are produced by a method of in vitro transcription comprising the steps of (a) providing a DNA vector encoding any of the encrypted RNAs described herein, (b) linearizing the DNA vector to produce a linear DNA vector; and (c) contacting the linear DNA vector with a RNA polymerase (e.g., at about 50° C.), thereby producing the isolated RNA polynucleotide. In some embodiments, the contacting of (c) is performed in the presence of one or more additional factors, e.g., ribonucleotide triphosphates, modified nucleotide triphosphates, a cap analog, inorganic pyrophosphatase, and a RNAse inhibitor).

In some embodiments, the method further comprises (d) subjecting the isolated RNA polynucleotide of (c) to one or more purification steps, e.g., contacting the isolated RNA polynucleotide with DNAse under conditions suitable for the digestion of the DNA vector; and tangential flow filtration.

In some embodiments, the DNA vector comprises a promoter capable of directing activity of the RNA polymerase and/or a restriction endonuclease recognition site. In some embodiments, the RNA polymerase is a T7 RNA polymerase and the promoter is a T7 promoter. In some embodiments, linearizing the DNA vector comprises contacting the DNA vector with a restriction endonuclease that recognizes the restriction endonuclease recognition site. In some embodiments, the method further comprises formulating the isolated RNA polynucleotide into a nanoparticle.

In some embodiments, the precursor encrypted RNA is comprised of an encrypted RNA portion and a ribozyme portion, in which the ribozyme portion cleaves the precursor encrypted RNA to generate two shorter RNA products, including the encrypted RNA and the ribozyme. In some embodiments, after cleavage of the precursor encrypted RNA by the ribozyme portion, the encrypted RNA is 5′-monophosphorylated.

Exemplary sequence elements of encrypted RNAs or of DNA-encoded encrypted RNAs are listed in Table 1. Exemplary pairings of sequence elements which can be used together as elements of an encrypted RNA are listed in Table 2. Exemplary coding sequences and reverse complements of coding sequences (i.e., antisense coding sequences) are listed in Table 3. Some useful sequences for producing some encrypted RNAs or for DNA-encoding encrypted RNAs are listed in Table 4. Some exemplary amino acid sequences of polypeptides of interest are listed in Table 5.

A table below should be read to continue, potentially for multiple pages, until the next table is listed or the tables end; e.g. Table 1 continues over multiple pages until Table 2 begins. Similarly, Table 5 continues until the next section entitled “Proteins of interest” begins. To the extent DNA sequences are listed, it is understood that the sequences also disclose and embody their RNA counterparts (T→U). Similarly, when RNA sequences are listed, it is understood that the sequences also disclose and embody their DNA counterparts (U→T).

TABLE 1
Sequence elements of L and R regions of encrypted RNAs

| SEQ | | | | | Se- | | |
|---|---|---|---|---|---|---|---|
| ID | Target | | Encryp- | | quence | | |
| NO: | Virus | Name | tion | Flank | Type | Length | Sequence |
| 1 | influenza | antis_5p_IAV | anti- | L | DNA or | 45 | agtagaaacaagggtgtttttcctcatatctctgaaattctaatc |
| | | | sense | | RNA | | |
| 2 | influenza | antis_5p_IAV_ | anti- | L | DNA or | 34 | agtagaaacaaggtcgtttttaaactattcgaca |
| | | seg01 | sense | | RNA | | |
| 3 | influenza | antis_5p_IAV_ | anti- | L | DNA or | 43 | agtagaaacaaggcattttttcatgaaggacaagctaaattca |
| | | seg02 | sense | | RNA | | |
| 4 | influenza | antis_5p_IAV_ | anti- | L | DNA or | 58 | agtagaaacaaggtacttttttggacagtatggatagcaaatagtagcactgccacaa |
| | | gse03 | sense | | RNA | | |
| 5 | influenza | antis_5p_IAV_ | anti- | L | DNA or | 45 | agtagaaacaagggtgtttttcctcatatttctgaaattctaatc |
| | | gse04 | sense | | RNA | | |
| 6 | influenza | antis_5p_IAV_ | anti- | L | DNA or | 23 | agtagaaacaagggtatttttct |
| | | seg05 | sense | | RNA | | |
| 7 | influenza | antis_5p_IAV_ | anti- | L | DNA or | 28 | agtagaaacaaggagttttttgaacaga |
| | | seg06 | sense | | RNA | | |
| 8 | influenza | antis_5p_IAV_ | anti- | L | DNA or | 20 | agtagaaacaaggtagtttt |
| | | seg07 | sense | | RNA | | |
| 9 | influenza | antis_5p_IAV_ | anti- | L | DNA or | 26 | agtagaaacaagggtgttttttatta |
| | | seg08 | sense | | RNA | | |
| 10 | influenza | antis_5p_IBV | anti- | L | DNA or | 94 | agtagtaacaagagcatttttcaataacgtttctttgtaatgacaacaagtaaacaagcactacaataaaggaaaata |
| | | | sense | | RNA | | cagggcttaacttccc |
| 11 | influenza | antis_5p_IBV_ | anti- | L | DNA or | 89 | agtagaaacacgagccttttttcattttaatcatttgtgtatcgcatgtattcaatgatgaccaataaccccataaaca |
| | | seg01 | sense | | RNA | | tcttcgaagc |
| 12 | influenza | antis_5p_IBV_ | anti- | L | DNA or | 60 | agtagaaacacgagcatttttcactcaattgcgttcattgaattaatggataaatttata |
| | | seg02 | sense | | RNA | | |
| 13 | influenza | antis_5p_IBV_ | anti- | L | DNA or | 95 | agtagaaacacgtgcatttttgattcttggttctttttattggatgtttagatacataatgaacaaaatagtaccaaat |
| | | seg03 | sense | | RNA | | tgagtaccatttcctt |
| 14 | influenza | antis_5p_IBV_ | anti- | L | DNA or | 101 | agtagaaacaacagcattttttatatttattttaataagaataaacacccacattccaaacgtattgaaacaatcacag |
| | | seg05 | sense | | RNA | | tcatagtgtctattttgttgcc |
| 15 | influenza | antis_5p_IBV_ | anti- | L | DNA or | 103 | agtagtaacaagagcatttttcagaaacaattaagtttagtaaggacaattgttcaaacaaaacaggaacaaagggtt |
| | | seg06 | sense | | RNA | | tagaacagactcaaccattcctcca |
| 16 | influenza | antis_5p_IBV_ | anti- | L | DNA or | 89 | agtagaaacaacgcactttttccagtttatttgctgacattgattacaatttgcttaaatgcatagtaagaaatacagt |
| | | seg07 | sense | | RNA | | aaaattgaat |
| 17 | influenza | antis_5p_IBV_ | anti- | L | DNA or | 30 | agtagtaacaagaggatttttattttaaat |
| | | seg08 | sense | | RNA | | |
| 18 | influenza | antis_3p_IAV | anti- | R | DNA or | 32 | tttggttgtttttattttcccctgcttttgct |
| | | | sense | | RNA | | |
| 19 | influenza | antis_3p_IAV_ | anti- | R | DNA or | 32 | tttggttgtttttattttcccctgtttctact |
| | | enhanced | sense | | RNA | | |
| 20 | influenza | antis_3p_IAV_ | anti- | R | DNA or | 27 | attgaatataattgacctgctttcgct |
| | | seg01 | sense | | RNA | | |
| 21 | influenza | antis_3p_IAV_ | anti- | R | DNA or | 27 | attgaatataattgacctgtttccact |
| | | seg01_enhanced | sense | | RNA | | |
| 22 | influenza | antis_3p_IAV_ | anti- | R | DNA or | 27 | attgaatataattgacctgcttttgct |
| | | seg01_m4 | sense | | RNA | | |
| 23 | influenza | antis_3p_IAV_ | anti- | R | DNA or | 27 | attgaatataattgacctgtttctact |
| | | seg01_m4_ | sense | | RNA | | |
| | | enhanced | | | | | |
| 24 | influenza | antis_3p_IAV_ | anti- | R | DNA or | 24 | tcaaatggtttgcctgctttcgct |
| | | seg02 | sense | | RNA | | |
| 25 | influenza | antis_3p_IAV_ | anti- | R | DNA or | 24 | tcaaatggtttgcctgtttccact |
| | | seg02_enhanced | sense | | RNA | | |
| 26 | influenza | antis_3p_IAV_ | anti- | R | DNA or | 24 | tcaaatggtttgcctgcttttgct |
| | | seg02_m4 | sense | | RNA | | |
| 27 | influenza | antis_3p_IAV_ | anti- | R | DNA or | 24 | tcaaatggtttgcctgtttctact |
| | | seg02_m4_ | sense | | RNA | | |
| | | enhanced | | | | | |
| 28 | influenza | antis_3p_IAV_ | anti- | R | DNA or | 24 | tttggatcagtacctgctttcgct |
| | | seg03 | sense | | RNA | | |
| 29 | influenza | antis_3p_IAV_ | anti- | R | DNA or | 24 | tttggatcagtacctgtttccact |
| | | seg03_enhanced | sense | | RNA | | |
| 30 | influenza | antis_3p_IAV_ | anti- | R | DNA or | 24 | tttggatcagtacctgcttttgct |
| | | seg03_m4 | sense | | RNA | | |
| 31 | influenza | antis_3p_IAV_ | anti- | R | DNA or | 24 | tttggatcagtacctgtttctact |
| | | seg03_m4_ | sense | | RNA | | |
| | | enhanced | | | | | |
| 32 | influenza | antis_3p_IAV_ | anti- | R | DNA or | 45 | gattttgatgtcactcagtgagtgattatctaccctgcttttgct |
| | | gse05 | sense | | RNA | | |
| 33 | influenza | antis_3p_IAV_ | anti- | R | DNA or | 45 | gattttgatgtcactcagtgagtgattatctaccctgtttctact |
| | | seg05_enhanced | sense | | RNA | | |
| 34 | influenza | antis_3p_IAV_ | anti- | R | DNA or | 20 | tttaaacccctgctttcgct |
| | | seg06 | sense | | RNA | | |
| 35 | influenza | antis_3p_IAV_ | anti- | R | DNA or | 20 | tttaaacccctgtttccact |
| | | seg06_enhanced | sense | | RNA | | |
| 36 | influenza | antis_3p_IAV_ | anti- | R | DNA or | 20 | tttaaacccctgcttttgct |
| | | seg06_m4 | sense | | RNA | | |
| 37 | influenza | antis_3p_IAV_ | anti- | R | DNA or | 20 | tttaaacccctgtttctact |
| | | seg06_m4_ | sense | | RNA | | |
| | | enhanced | | | | | |
| 38 | influenza | antis_3p_IAV_ | anti- | R | DNA or | 25 | ctttcaatatctacctgctttcgct |
| | | seg07 | sense | | RNA | | |
| 39 | influenza | antis_3p_IAV_ | anti- | R | DNA or | 25 | ctttcaatatctacctgtttccact |
| | | gse07_enhanced | sense | | RNA | | |
| 40 | influenza | antis_3p_IAV_ | anti- | R | DNA or | 25 | ctttcaatatctacctgcttttgct |
| | | seg07_m4 | sense | | RNA | | |
| 41 | influenza | antis_3p_IAV_ | anti- | R | DNA or | 25 | ctttcaatatctacctgtttctact |
| | | seg07_m4_ | sense | | RNA | | |
| | | enhanced | | | | | |
| 42 | influenza | antis_3p_IAV_ | anti- | R | DNA or | 26 | tatgtctttgtcaccctgcttttgct |
| | | seg08 | sense | | RNA | | |
| 43 | influenza | antis_3p_IAV_ | anti- | R | DNA or | 26 | tatgtctttgtcaccctgtttctact |
| | | seg08_enhanced | sense | | RNA | | |
| 44 | influenza | antis_3p_IBV | anti- | R | DNA or | 33 | tttgtggatattagaaaatgctctgcttctgct |
| | | | sense | | RNA | | |
| 45 | influenza | antis_3p_IBV_en | anti- | R | DNA or | 33 | tttgtggatattagaaaatgctctgtttctact |
| | | hanced | sense | | RNA | | |
| 46 | influenza | antis_3p_IBV_ | anti- | R | DNA or | 21 | cttaaaggctccgcttctgct |
| | | seg01 | sense | | RNA | | |
| 47 | influenza | antis_3p_IBV_ | anti- | R | DNA or | 21 | cttaaaggctccgtttctact |
| | | seg01_enhanced | sense | | RNA | | |
| 48 | influenza | antis_3p_IBV_ | anti- | R | DNA or | 23 | cttgaaaacgctccgcttctgct |
| | | seg02 | sense | | RNA | | |
| 49 | influenza | antis_3p_IBV_ | anti- | R | DNA or | 23 | cttgaaaacgctccgtttctact |
| | | seg02_enhanced | sense | | RNA | | |
| 50 | influenza | antis_3p_IBV_ | anti- | R | DNA or | 29 | tatgacaaatcaaacgcaccgcttctgct |
| | | seg03 | sense | | RNA | | |
| 51 | influenza | antis_3p_IBV_ | anti- | R | DNA or | 29 | tatgacaaatcaaacgcaccgtttctact |
| | | seg03_enhanced | sense | | RNA | | |
| 52 | influenza | antis_3p_IBV_ | anti- | R | DNA or | 60 | tttgattttcagttcttttactggtgcttgaagttcacaagaaaatgctgtgcttctgct |
| | | seg05 | sense | | RNA | | |
| 53 | influenza | antis_3p_IBV_ | anti- | R | DNA or | 60 | tttgattttcagttcttttactggtgcttgaagttcacaagaaaatgctgtgtttctact |
| | | seg05_enhanced | sense | | RNA | | |
| 54 | influenza | antis_3p_IBV_ | anti- | R | DNA or | 46 | ttttggcctatttgcctcagttttgagaagatgctctgcttctgct |
| | | seg60 | sense | | RNA | | |
| 55 | influenza | antis_3p_IBV_ | anti- | R | DNA or | 46 | ttttggcctatttgcctcagttttgagaagatgctctgtttctact |
| | | seg06_enhanced | sense | | RNA | | |
| 56 | influenza | antis_3p_IBV_ | anti- | R | DNA or | 24 | tttaagaaagtgcgtgcttctgct |
| | | seg07 | sense | | RNA | | |
| 57 | influenza | antis_3p_IBV_ | anti- | R | DNA or | 24 | tttaagaaagtgcgtgtttctact |
| | | seg07_enhanced | sense | | RNA | | |
| 58 | influenza | antis_3p_IBV_ | anti- | R | DNA or | 44 | ttttccctgtttgccagtgactaaacaaatcctctgcttctgct |
| | | seg08 | sense | | RNA | | |
| 59 | influenza | antis_3p_IBV_ | anti- | R | DNA or | 44 | ttttccctgtttgccagtgactaaacaaatcctctgcttctact |
| | | seg08_enhanced | sense | | RNA | | |
| 60 | sarbe- | sense_5p_SARS1 | sense | L | DNA or | 1493 | atattaggtttttacctacccaggaaaagccaaccaacctcgatctcttgtagatctgttctctaaacgaactttaaaa |
| | covirus | | | | RNA | | tctgtgtagctgtcgctcggctgcatgcctagtgcacctacgcagtataaacaataataaattttactgtcgttgacaa |
| | | | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagt |
| | | | | | | | ttgcctgtccttcaggttagagacgtgctagtgcgtggcttcggggactctgtggaagaggccctatcggaggcac |
| | | | | | | | gtgaacacctcaaaaatggcacttgtggtctagtagagctggaaaaaggcgtactgccccagcttgaacagcccta |
| | | | | | | | tgtgttcattaaacgttctgatgccttaagcaccaatcacggccacaaggtcgttgagctggttgcagaaatggacg |
| | | | | | | | gcattcagtacggtcgtagcggtataacactgggagtactcgtgccacatgtgggcgaaaccccaattgcataccg |
| | | | | | | | caatgttcttcttcgtaagaacggtaataagggagccggtggtcatagctatggcatcgatctaaagtcttatgactta |
| | | | | | | | ggtgacgagcttggcactgatcccattgaagattatgaacaaaactggaacactaagcatggcagtggtgcactcc |
| | | | | | | | gtgaactcactcgcgccggcgaagcacctgtttccatcattaataatgctgtttacacaaaggtagatggtattgatgt |
| | | | | | | | ggagatctttgaaaataagacaacacttcctgttaatgttgcatttgagctttgggctaagcgtaacattaaaccagtg |
| | | | | | | | ccagagattaagatactcaataatttgggtgttgatatcgctgctaatactgtaatctgggactacaaaagagaagcc |
| | | | | | | | ccagcacatgtatctacaataggtgtctgcacaatgactgacattgccaagaaacctactgagagtgcttgttcttca |
| | | | | | | | cttactgtcttgtttgatggtagagtggaaggacaggtagacctttttagaaacgcccgtaatggtgttttaataacag |
| | | | | | | | aaggttcagtcaaaggtctaacaccttcaaagggaccagcacaagctagcgtcaatggagtcacattaattggaga |
| | | | | | | | atcagtaaaaacacagtttaactactttaagaaagtagacggcattattcaacagttgcctgaaacctactttactcag |
| | | | | | | | agcagagacttagaggattttaagcccagatcacaaatggaaactgactttctcgagctcgctatggatgaattcata |
| | | | | | | | cagcgatataagctcgagggctatgccttcgaacacatcgtttatggagatttctgacgtacttgttgttttaaataaa |
| | | | | | | | cgaacaaattaaa |
| 61 | sarbe- | sense_5p_SARS1 | sense | L | DNA or | 1493 | atattaggtttttacctacccaggaaaagccaaccaacctcgatctcttgtagatctgttctctaaccggattttaaaa |
| | covirus | GL | | | RNA | | tctgtgtagctgtcgctcggctgcatgcctagtgcacctacgcagtataaacaataataaattttactgtcgttgacaa |
| | | | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagtt |
| | | | | | | | tgcctgtccttcaggttagagacgtgctagtgcgtggcttcggggactctgtggaagaggccctatcggaggcacg |
| | | | | | | | tgaacacctcaaaaatggcacttgtggtctagtagagctggaaaaaggcgtactgccccagcttgaacagccctat |
| | | | | | | | gtgttcattaaacgttctgatgccttaagcaccaatcacggccacaaggtcgttgagctggttgcagaaatggacgg |
| | | | | | | | cattcagtacggtcgtagcggtataacactgggagtactcgtgccacatgtgggcgaaaccccaattgcataccgc |
| | | | | | | | aatgttcttcttcgtaagaacggtaataagggagccggtggtcatagctatggcatcgatctaaagtcttatgacttag |
| | | | | | | | gtgacgagcttggcactgatcccattgaagattatgaacaaaactggaacactaagcatggcagtggtgcactccg |
| | | | | | | | tgaactcactcgcgccggcgaagcacctgtttccatcattaataatgctgtttacacaaaggtagatggtattgatgtg |
| | | | | | | | gagatctttgaaaataagacaacacttcctgttaatgttgcatttgagctttgggctaagcgtaacattaaaccagtgc |
| | | | | | | | cagagattaagatactcaataatttgggtgttgatatcgctgctaatactgtaatctgggactacaaaagagaagccc |
| | | | | | | | cagcacatgtatctacaataggtgtctgcacaatgactgacattgccaagaaacctactgagagtgcttgttcttcact |
| | | | | | | | tactgtcttgtttgatggtagagtggaaggacaggtagacctttttagaaacgcccgtaatggtgttttaataacagaa |
| | | | | | | | ggttcagtcaaaggtctaacaccttcaaagggaccagcacaagctagcgtcaatggagtcacattaattggagaat |
| | | | | | | | cagtaaaaacacagtttaactactttaagaaagtagacggcattattcaacagttgcctgaaacctactttactcagag |
| | | | | | | | cagagacttagaggattttaagcccagatcacaaatggaaactgactttctcgagctcgctatggatgaattcataca |
| | | | | | | | gcgatataagctcgagggctatgccttcgaacacatcgtttatggagatttctgacgtacttgttgttttaaataaccg |
| | | | | | | | gataaattaaa |
| 62 | sarbe- | sense_5p_SARS1 | sense | L | DNA or | 1493 | atattaggtttttacctacccaggaaaagccaaccaacctcgatctcttgtagatctgttctctaaccggattttaaaa |
| | covirus | GL_nsp1_124 | | | RNA | | tctgtgtagctgtcgctcggctgcatgcctagtgcacctacgcagtataaacaataataaattttactgtcgttgacaa |
| | | | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagtt |
| | | | | | | | tgcctgtccttcaggttagagacgtgctagtgcgtggcttcggggactctgtggaagaggccctatcggaggcacg |
| | | | | | | | tgaacacctcaaaaatggcacttgtggtctagtagagctggaaaaaggcgtactgccccagcttgaacagccctat |
| | | | | | | | gtgttcattaaacgttctgatgccttaagcaccaatcacggccacaaggtcgttgagctggttgcagaaatggacgg |
| | | | | | | | cattcagtacggtcgtagcggtataacactgggagtactcgtgccacatgtgggcgaaaccccaattgcataccgc |
| | | | | | | | aatgttcttcttgctgccaacggtaataagggagccggtggtcatagctatggcatcgatctaaagtcttatgacttag |
| | | | | | | | gtgacgagcttggcactgatcccattgaagattatgaacaaaactggaacactaagcatggcagtggtgcactccg |
| | | | | | | | tgaactcactcgcgccggcgaagcacctgtttccatcattaataatgctgtttacacaaaggtagatggtattgatgtg |
| | | | | | | | gagatctttgaaaataagacaacacttcctgttaatgttgcatttgagctttgggctaagcgtaacattaaaccagtgc |
| | | | | | | | cagagattaagatactcaataatttgggtgttgatatcgctgctaatactgtaatctgggactacaaaagagaagccc |
| | | | | | | | cagcacatgtatctacaataggtgtctgcacaatgactgacattgccaagaaacctactgagagtgcttgttcttcact |
| | | | | | | | tactgtcttgtttgatggtagagtggaaggacaggtagacctttttagaaacgcccgtaatggtgttttaataacagaa |
| | | | | | | | ggttcagtcaaaggtctaacaccttcaaagggaccagcacaagctagcgtcaatggagtcacattaattggagaat |
| | | | | | | | cagtaaaaacacagtttaactactttaagaaagtagacggcattattcaacagttgcctgaaacctactttactcagag |
| | | | | | | | cagagacttagaggattttaagcccagatcacaaatggaaactgactttctcgagctcgctatggatgaattcataca |
| | | | | | | | gcgatataagctcgagggctatgccttcgaacacatcgtttatggagatttctgacgtacttgttgttttaaataaacg |
| | | | | | | | aacaaattaaa |
| 63 | sarbe- | sense_5p_SARS1 | sense | L | DNA or | 1493 | atattaggtttttacctacccaggaaaagccaaccaacctcgatctcttgtagatctgttctctaaccggattttaaaa |
| | covirus | GL_nsp1_124_ | | | RNA | | tctgtgtagctgtcgctcggctgcatgcctagtgcacctacgcagtataaacaataataaattttactgtcgttgacaa |
| | | 164 | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagtt |
| | | | | | | | tgcctgtccttcaggttagagacgtgctagtgcgtggcttcggggactctgtggaagaggccctatcggaggcacg |
| | | | | | | | tgaacacctcaaaaatggcacttgtggtctagtagagctggaaaaaggcgtactgccccagcttgaacagccctat |
| | | | | | | | gtgttcattaaacgttctgatgccttaagcaccaatcacggccacaaggtcgttgagctggttgcagaaatggacgg |
| | | | | | | | cattcagtacggtcgtagcggtataacactgggagtactcgtgccacatgtgggcgaaaccccaattgcataccgc |
| | | | | | | | aatgttcttcttgctgccaacggtaataagggagccggtggtcatagctatggcatcgatctaaagtcttatgacttag |
| | | | | | | | gtgacgagcttggcactgatcccattgaagattatgaacaaaactggaacactgccgctggcagtggtgcactccg |
| | | | | | | | tgaactcactcgcgccggcgaagcacctgtttccatcattaataatgctgtttacacaaaggtagatggtattgatgtg |
| | | | | | | | gagatctttgaaaataagacaacacttcctgttaatgttgcatttgagctttgggctaagcgtaacattaaaccagtgc |
| | | | | | | | cagagattaagatactcaataatttgggtgttgatatcgctgctaatactgtaatctgggactacaaaagagaagccc |
| | | | | | | | cagcacatgtatctacaataggtgtctgcacaatgactgacattgccaagaaacctactgagagtgcttgttcttcact |
| | | | | | | | tactgtcttgtttgatggtagagtggaaggacaggtagacctttttagaaacgcccgtaatggtgttttaataacagaa |
| | | | | | | | ggttcagtcaaaggtctaacaccttcaaagggaccagcacaagctagcgtcaatggagtcacattaattggagaat |
| | | | | | | | cagtaaaaacacagtttaactactttaagaaagtagacggcattattcaacagttgcctgaaacctactttactcagag |
| | | | | | | | cagagacttagaggattttaagcccagatcacaaatggaaactgactttctcgagctcgctatggatgaattcataca |
| | | | | | | | gcgatataagctcgagggctatgccttcgaacacatcgtttatggagatttctgacgtacttgttgttttaaataaccg |
| | | | | | | | gataaattaaa |
| 64 | sarbe- | sense_5p_SARS1 | sense | L | DNA or | 1493 | atattaggtttttacctacccaggaaaagccaaccaacctcgatctcttgtagatctgttctctaaccggattttaaaa |
| | covirus | GL_nsp1_164 | | | RNA | | tctgtgtagctgtcgctcggctgcatgcctagtgcacctacgcagtataaacaataataaattttactgtcgttgacaa |
| | | | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagtt |
| | | | | | | | tgcctgtccttcaggttagagacgtgctagtgcgtggcttcggggactctgtggaagaggccctatcggaggcacg |
| | | | | | | | tgaacacctcaaaaatggcacttgtggtctagtagagctggaaaaaggcgtactgccccagcttgaacagccctat |
| | | | | | | | gtgttcattaaacgttctgatgccttaagcaccaatcacggccacaaggtcgttgagctggttgcagaaatggacgg |
| | | | | | | | cattcagtacggtcgtagcggtataacactgggagtactcgtgccacatgtgggcgaaaccccaattgcataccgc |
| | | | | | | | aatgttcttcttcgtaagaacggtaataagggagccggtggtcatagctatggcatcgatctaaagtcttatgacttag |
| | | | | | | | gtgacgagcttggcactgatcccattgaagattatgaacaaaactggaacactgccgctggcagtggtgcactccg |
| | | | | | | | tgaactcactcgcgccggcgaagcacctgtttccatcattaataatgctgtttacacaaaggtagatggtattgatgtg |
| | | | | | | | gagatctttgaaaataagacaacacttcctgttaatgttgcatttgagctttgggctaagcgtaacattaaaccagtgc |
| | | | | | | | cagagattaagatactcaataatttgggtgttgatatcgctgctaatactgtaatctgggactacaaaagagaagccc |
| | | | | | | | cagcacatgtatctacaataggtgtctgcacaatgactgacattgccaagaaacctactgagagtgcttgttcttcact |
| | | | | | | | tactgtcttgtttgatggtagagtggaaggacaggtagacctttttagaaacgcccgtaatggtgttttaataacagaa |
| | | | | | | | ggttcagtcaaaggtctaacaccttcaaagggaccagcacaagctagcgtcaatggagtcacattaattggagaat |
| | | | | | | | cagtaaaaacacagtttaactactttaagaaagtagacggcattattcaacagttgcctgaaacctactttactcagag |
| | | | | | | | cagagacttagaggattttaagcccagatcacaaatggaaactgactttctcgagctcgctatggatgaattcataca |
| | | | | | | | gcgatataagctcgagggctatgccttcgaacacatcgtttatggagatttctgacgtacttgttgttttaaataaccg |
| | | | | | | | gataaattaaa |
| 65 | sarbe- | sense_5p_ | sense | L | DNA or | 1493 | atattaggtttttacctacccaggaaaagccaaccaacctcgatctcttgtagatctgttctctaaacgaactttaaaa |
| | covirus | SARS1_nsp1_124 | | | RNA | | tctgtgtagctgtcgctcggctgcatgcctagtgcacctacgcagtataaacaataataaattttactgtcgttgacaa |
| | | | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagt |
| | | | | | | | ttgcctgtccttcaggttagagacgtgctagtgcgtggcttcggggactctgtggaagaggccctatcggaggcac |
| | | | | | | | gtgaacacctcaaaaatggcacttgtggtctagtagagctggaaaaaggcgtactgccccagcttgaacagcccta |
| | | | | | | | tgtgttcattaaacgttctgatgccttaagcaccaatcacggccacaaggtcgttgagctggttgcagaaatggacg |
| | | | | | | | gcattcagtacggtcgtagcggtataacactgggagtactcgtgccacatgtgggcgaaaccccaattgcataccg |
| | | | | | | | caatgttcttcttgctgccaacggtaataagggagccggtggtcatagctatggcatcgatctaaagtcttatgactta |
| | | | | | | | ggtgacgagcttggcactgatcccattgaagattatgaacaaaactggaacactaagcatggcagtggtgcactcc |
| | | | | | | | gtgaactcactcgcgccggcgaagcacctgtttccatcattaataatgctgtttacacaaaggtagatggtattgatgt |
| | | | | | | | ggagatctttgaaaataagacaacacttcctgttaatgttgcatttgagctttgggctaagcgtaacattaaaccagtg |
| | | | | | | | ccagagattaagatactcaataatttgggtgttgatatcgctgctaatactgtaatctgggactacaaaagagaagcc |
| | | | | | | | ccagcacatgtatctacaataggtgtctgcacaatgactgacattgccaagaaacctactgagagtgcttgttcttca |
| | | | | | | | cttactgtcttgtttgatggtagagtggaaggacaggtagacctttttagaaacgcccgtaatggtgttttaataacag |
| | | | | | | | aaggttcagtcaaaggtctaacaccttcaaagggaccagcacaagctagcgtcaatggagtcacattaattggaga |
| | | | | | | | atcagtaaaaacacagtttaactactttaagaaagtagacggcattattcaacagttgcctgaaacctactttactcag |
| | | | | | | | agcagagacttagaggattttaagcccagatcacaaatggaaactgactttctcgagctcgctatggatgaattcata |
| | | | | | | | cagcgatataagctcgagggctatgccttcgaacacatcgtttatggagatttctgacgtacttgttgttttaaataaa |
| | | | | | | | cgaacaaattaaa |
| 66 | sarbe- | sense_5p_ | sense | L | DNA or | 1493 | atattaggtttttacctacccaggaaaagccaaccaacctcgatctcttgtagatctgttctctaaacgaactttaaaa |
| | covirus | SARS1_nsp1_ | | | RNA | | tctgtgtagctgtcgctcggctgcatgcctagtgcacctacgcagtataaacaataataaattttactgtcgttgacaa |
| | | 124_164 | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagt |
| | | | | | | | ttgcctgtccttcaggttagagacgtgctagtgcgtggcttcggggactctgtggaagaggccctatcggaggcac |
| | | | | | | | gtgaacacctcaaaaatggcacttgtggtctagtagagctggaaaaaggcgtactgccccagcttgaacagcccta |
| | | | | | | | tgtgttcattaaacgttctgatgccttaagcaccaatcacggccacaaggtcgttgagctggttgcagaaatggacg |
| | | | | | | | gcattcagtacggtcgtagcggtataacactgggagtactcgtgccacatgtgggcgaaaccccaattgcataccg |
| | | | | | | | caatgttcttcttgctgccaacggtaataagggagccggtggtcatagctatggcatcgatctaaagtcttatgactta |
| | | | | | | | ggtgacgagcttggcactgatcccattgaagattatgaacaaaactggaacactgccgctggcagtggtgcactcc |
| | | | | | | | gtgaactcactcgcgccggcgaagcacctgtttccatcattaataatgctgtttacacaaaggtagatggtattgatgt |
| | | | | | | | ggagatctttgaaaataagacaacacttcctgttaatgttgcatttgagctttgggctaagcgtaacattaaaccagtg |
| | | | | | | | ccagagattaagatactcaataatttgggtgttgatatcgctgctaatactgtaatctgggactacaaaagagaagcc |
| | | | | | | | ccagcacatgtatctacaataggtgtctgcacaatgactgacattgccaagaaacctactgagagtgcttgttcttca |
| | | | | | | | cttactgtcttgtttgatggtagagtggaaggacaggtagacctttttagaaacgcccgtaatggtgttttaataacag |
| | | | | | | | aaggttcagtcaaaggtctaacaccttcaaagggaccagcacaagctagcgtcaatggagtcacattaattggaga |
| | | | | | | | atcagtaaaaacacagtttaactactttaagaaagtagacggcattattcaacagttgcctgaaacctactttactcag |
| | | | | | | | agcagagacttagaggattttaagcccagatcacaaatggaaactgactttctcgagctcgctatggatgaattcata |
| | | | | | | | cagcgatataagctcgagggctatgccttcgaacacatcgtttatggagatttctgacgtacttgttgttttaaataaa |
| | | | | | | | cgaacaaattaaa |
| 67 | sarbe- | sense_5p_ | sense | L | DNA or | 1493 | atattaggtttttacctacccaggaaaagccaaccaacctcgatctcttgtagatctgttctctaaacgaactttaaaa |
| | covirus | SARS1_nsp1_164 | | | RNA | | tctgtgtagctgtcgctcggctgcatgcctagtgcacctacgcagtataaacaataataaattttactgtcgttgacaa |
| | | | | | | | gaaacgagtaactcgtccctcttctgcagactgcttacggtttcgtccgtgttgcagtcgatcatcagcatacctaggt |
| | | | | | | | ttcgtccgggtgtgaccgaaaggtaagatggagagccttgttcttggtgtcaacgagaaaacacacgtccaactcagt |
| | | | | | | | ttgcctgtccttcaggttagagacgtgctagtgcgtggcttcggggactctgtggaagaggccctatcggaggcac |
| | | | | | | | gtgaacacctcaaaaatggcacttgtggtctagtagagctggaaaaaggcgtactgccccagcttgaacagcccta |
| | | | | | | | tgtgttcattaaacgttctgatgccttaagcaccaatcacggccacaaggtcgttgagctggttgcagaaatggacg |
| | | | | | | | gcattcagtacggtcgtagcggtataacactgggagtactcgtgccacatgtgggcgaaaccccaattgcataccg |
| | | | | | | | caatgttcttcttcgtaagaacggtaataagggagccggtggtcatagctatggcatcgatctaaagtcttatgactta |
| | | | | | | | ggtgacgagcttggcactgatcccattgaagattatgaacaaaactggaacactgccgctggcagtggtgcactcc |
| | | | | | | | gtgaactcactcgcgccggcgaagcacctgtttccatcattaataatgctgtttacacaaaggtagatggtattgatgt |
| | | | | | | | ggagatctttgaaaataagacaacacttcctgttaatgttgcatttgagctttgggctaagcgtaacattaaaccagtg |
| | | | | | | | ccagagattaagatactcaataatttgggtgttgatatcgctgctaatactgtaatctgggactacaaaagagaagcc |
| | | | | | | | ccagcacatgtatctacaataggtgtctgcacaatgactgacattgccaagaaacctactgagagtgcttgttcttca |
| | | | | | | | cttactgtcttgtttgatggtagagtggaaggacaggtagacctttttagaaacgcccgtaatggtgttttaataacag |
| | | | | | | | aaggttcagtcaaaggtctaacaccttcaaagggaccagcacaagctagcgtcaatggagtcacattaattggaga |
| | | | | | | | atcagtaaaaacacagtttaactactttaagaaagtagacggcattattcaacagttgcctgaaacctactttactcag |
| | | | | | | | agcagagacttagaggattttaagcccagatcacaaatggaaactgactttctcgagctcgctatggatgaattcata |
| | | | | | | | cagcgatataagctcgagggctatgccttcgaacacatcgtttatggagatttctgacgtacttgttgttttaaataaa |
| | | | | | | | cgaacaaattaaa |
| 68 | sarbe- | sense_5p_SARS2 | sense | L | DNA or | 1501 | attaaaggtttataccttcccaggtaacaaaccaaccaactttcgatctcttgtagatctgttctctaaacgaacttta |
| | covirus | | | | RNA | | aaatctgtgtggctgtcactcggctgcatgcttagtgcactcacgcagtataattaataactaattactgtcgttgaca |
| | | | | | | | aggcacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaacga |
| | | | | | | | acaaactaaa |
| 69 | sarbe- | sense_5p_SARS2 | sense | L | DNA or | 1501 | attaaaggtttataccttcccaggtaacaaaccaaccaactttcgatctcttgtagatctgttctctaaccggatttta |
| | covirus | GL | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaccgg |
| | | | | | | | ataaactaaa |
| 70 | sarbe- | sense_5p_SARS2 | sense | L | DNA or | 1513 | attaaaggtttataccttcccaggtaacaaaccaaccaactttcgatctcttgtagatctgttctctaaccggatttta |
| | covirus | GL_ATG_HP15 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttctcccattttagtgacgttcgtgttgttttagatt |
| | | | | | | | tcatctaaccggataaactaaa |
| 71 | sarbe- | sense_5p_SARS2 | sense | L | DNA or | 1522 | attaaaggtttataccttcccaggtaacaaaccaaccaactttcgatctcttgtagatctgttctctaaccggatttta |
| | covirus | GL_ATG_HP30 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttctttgactcccattttagttttgacgttcgtgttg |
| | | | | | | | ttttagatttcatctaaccggataaactaaa |
| 72 | sarbe- | sense_5p_SARS2 | sense | L | DNA or | 1529 | attaaaggtttataccttcccaggtaacaaaccaaccaactttcgatctcttgtagatctgttctctaaccggatttta |
| | covirus | GL_ATG_HP45 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttgaactttgactcccattttagtttatcctgacgtt |
| | | | | | | | cgtgttgttttagatttcatctaaccggataaactaaa |
| 73 | sarbe- | sense_5p_SARS2 | sense | L | DNA or | 1536 | attaaaggtttataccttcccaggtaacaaaccaaccaactttcgatctcttgtagatctgttctctaaccggatttta |
| | covirus | GL_ATG_HP60 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | tgtgtaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttcaaacagaactttgactcccattttagtttatccg |
| | | | | | | | tgacgttcgtgttgttttagatttcatctaaccggataaactaaa |
| 74 | sarbe | sense_5p_SARS2 | sense | | DNA or | 1552 | taaacctttaatctgatgagtccgtgaggacgaaacccggagtcccgggtcattaaaggtttataccttcccaggtaa |
| | covirus | GL_GGC_HHRz | | | RNA | | caaaccaaccaactttcgatctcttgtagatctgttctctaaccggattttaaaatctgtgtggctgtcactcggctgc |
| | | | | | | | atgcttagtgcactcacgcagtataattaataactaattactgtcgttgacaggacacgagtaactcgtctatcttctg |
| | | | | | | | caggctgcttacggtttcgtccgtgttgcagccgatcatcagcacatctaggtttcgtccgggtgtgaccgaaaggtaa |
| | | | | | | | gatggagagccttgtccctggtttcaacgagaaaacacacgtccaactcagtttgcctgttttacaggttcgcgacgt |
| | | | | | | | gctcgtacgtggctttggagactccgtggaggaggtcttatcagaggcacgtcaacatcttaaagatggcacttgtg |
| | | | | | | | gcttagtagaagttgaaaaaggcgttttgcctcaacttgaacagccctatgtgttcatcaaacgttcggatgctcgaa |
| | | | | | | | ctgcacctcatggtcatgttatggttgagctggtagcagaactcgaaggcattcagtacggtcgtagtggtgagaca |
| | | | | | | | cttggtgtccttgtccctcatgtgggcgaaataccagtggcttaccgcaaggttcttcttcgtaagaacggtaataaa |
| | | | | | | | ggagctggtggccatagttacggcgccgatctaaagtcatttgacttaggcgacgagcttggcactgatccttatga |
| | | | | | | | agattttcaagaaaactggaacactaaacatagcagtggtgttacccgtgaactcatgcgacagggtgaagtacca |
| | | | | | | | gtttctatcattaataacactgtttacacaaaagttgatggtgttgatgtagaattgtttgaaaataaaacaacattac |
| | | | | | | | ctgttaatgtagcatttgagctttgggctaagcgcaacattaaaccagtaccagaggtgaaaatactcaataatttggg |
| | | | | | | | tgtggacattgctgctaatactgtgatctgggactacaaaagagatgctccagcacatatatctactattggtgtttgt |
| | | | | | | | tctatgactgacatagccaagaaaccaactgaaacgatttgtgcaccactcactgtcttttttgatggtagagttgatg |
| | | | | | | | gtcaagtagacttatttagaaatgcccgtaatggtgttcttattacagaaggtagtgttaaaggtttacaaccatctgt |
| | | | | | | | aggtcccaaacaagctagtcttaatggagtcacattaattggagaagccgtaaaaacacagttcaattattataagaaa |
| | | | | | | | gttgatggtgttgtccaacaattacctgaaacttactttactcagagtagaaatttacaagaatttaaacccaggagtc |
| | | | | | | | aaatggaaattgatttcttagaattagctatggatgaattcattgaacggtataaattagaaggctatgccttcgaaca |
| | | | | | | | tatcgtttatggagatttttgacgttcgtgttgttttagatttcatctaaccggataaactaaa |
| 75 | sarbe- | sense_5p_SARS2 | sense | L | DNA or | 1753 | attaaaggtttataccttcccaggtaacaaaccaaccaactttcgatctcttgtagatctgttctctaaccggatttta |
| | covirus | GL_N250 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggt |
| | | | | | | | agtgttaaaggtttacaaccatctgtaggtcccaaacaagctagtcttaatggagtcacattaattggagaagccgtaa |
| | | | | | | | aaacacagttcaattattataagaaagttgatggtgttgtccaacaattacctgaaacttactttactcagagtagaaa |
| | | | | | | | tttacaagaatttaaacccaggagtcaaatggaaattgatttcttagaattagctatggatgaattcattgaacggtat |
| | | | | | | | aaattagaaggctatgccttcgaacatatcgtttatggagattgacaaggcgttccaattaacaccaatagcagtcca |
| | | | | | | | gatgaccaaattggctactaccgaagagctaccagacgaattcgtggtggtgacggtaaaatgaaagatctcagtccaa |
| | | | | | | | gatggtatttctactacctaggaactgggccagaagctggacttccctatggtgctaacaaagacggcatcatatgggt |
| | | | | | | | tgcaactgagggagccttgaatacaccaaaagatcacattggcacccgcaatccttaaaactgacgttcgtgttgtttt |
| | | | | | | | agatttcatctaaccggataaactaaa |
| 76 | sarbe- | sense_5p_SARS2 | sense | L | DNA or | 1771 | attaaaggtttataccttcccaggtaacaaaccaaccaactttcgatctcttgtagatctgttctctaaccggatttta |
| | covirus | GL_N250_HP10 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattgacaaggcgttccaattaacaccaatagcagtccaga |
| | | | | | | | tgaccaaattggctactaccgaagagctaccagacgaattcgtggtggtgacggtaaaatgaaagatctcagtccaaga |
| | | | | | | | tggtatttctactacctaggaactgggccagaagctggacttccctatggtgctaacaaagacggcatcatatgggtt |
| | | | | | | | gcaactgagggagccttgaatacaccaacggccgatatcacggccgaagatcacattggcacccgcaatccttaa |
| | | | | | | | aactgacgttcgtgttgttttagatttcatctaaccggataaactaaa |
| 77 | sarbe- | sense_5p_SARS2 | sense | L | DNA or | 1787 | attaaaggtttataccttcccaggtaacaaaccaaccaactttcgatctcttgtagatctgttctctaaccggatttta |
| | covirus | GL_N250_HP30 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaa |
| | | | | | | | aaacacagttcaattattataagaaagttgatggtgttgtccaacaattacctgaaacttactttactcagagtagaaa |
| | | | | | | | tttacaagaatttaaacccaggagtcaaatggaaattgatttcttagaattagctatggatgaattcattgaacggtat |
| | | | | | | | aaattagaaggctatgccttcgaacatatcgtttatggagattgacaaggcgttccaattaacaccaatagcagtccag |
| | | | | | | | atgaccaaattggctactaccgaagagctaccagacgaattcgtggtggtgacggtaaaatgaaagatctcagtccaa |
| | | | | | | | gatggtatttctactacctaggaactgggccagaagctggacttccctatggtgctaacaaagacggcatcatatgggt |
| | | | | | | | tgcaactgagggagccttgaatacaccaagtccaccacggccgatatcacggccgtggtggacaagatcacattg |
| | | | | | | | gcacccgcaatccttaaaactgacgttcgtgttgttttagatttcatctaaccggataaactaaa |
| 78 | sarbe- | sense_5p_SARS2 | sense | L | DNA or | 1801 | attaaaggtttataccttcccaggtaacaaaccaaccaactttcgatctcttgtagatctgttctctaaccggatttta |
| | covirus | GL_N250_HP50 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtat |
| | | | | | | | aaattagaaggctatgccttcgaacatatcgtttatggagattgacaaggcgttccaattaacaccaatagcagtccag |
| | | | | | | | atgaccaaattggctactaccgaagagctaccagacgaattcgtggtggtgacggtaaaatgaaagatctcagtccaa |
| | | | | | | | gatggtatttctactacctaggaactgggccagaagctggacttccctatggtgctaacaaagacggcatcatatgggt |
| | | | | | | | tgcaactgagggagccttgaatacaccaaccctgcggtccaccacggccgatatcacggccgtggtggaccgca |
| | | | | | | | gggaagatcacattggcacccgcaatccttaaaactgacgttcgtgttgttttagatttcatctaaccggataaactaa |
| | | | | | | | a |
| 79 | sarbe- | sense_5p_SARS2 | sense | L | DNA or | 1754 | attaaaggtttataccttcccaggtaacaaaccaaccaactttcgatctcttgtagatctgttctctaaccggatttt |
| | covirus | GL_N250_stop | | | RNA | | aaaatctgtgtggctgtcactcggctgcatgcttagtgcactcacgcagtataattaataactaattactgtcgttgac |
| | | | | | | | aggacacgagtaactcgtctatcttctgcaggctgcttacggtttcgtccgtgttgcagccgatcatcagcacatcta |
| | | | | | | | ggtttcgtccgggtgtgaccgaaaggtaagatggagagccttgtccctggtttcaacgagaaaacacacgtccaactca |
| | | | | | | | gtttgcctgtttaaacaggttcgcgacgtgctcgtacgtggctttggagactccgtggaggaggtcttatcagaggc |
| | | | | | | | acgtcaacatcttaaagatggcacttgtggcttagtagaagttgaaaaaggcgttttgcctcaacttgaacagcccta |
| | | | | | | | tgtgttcatcaaacgttcggatgctcgaactgcacctcatggtcatgttatggttgagctggtagcagaactcgaagg |
| | | | | | | | cattcagtacggtcgtagtggtgagacacttggtgtccttgtccctcatgtgggcgaaataccagtggcttaccgca |
| | | | | | | | aggttcttcttcgtaagaacggtaataaaggagctggtggccatagttacggcgccgatctaaagtcatttgacttag |
| | | | | | | | gcgacgagcttggcactgatccttatgaagattttcaagaaaactggaacactaaacatagcagtggtgttacccgt |
| | | | | | | | gaactcatgcgacagggtgaagtaccagtttctatcattaataacactgtttacacaaaagttgatggtgttgatgtag |
| | | | | | | | aattgtttgaaaataaaacaacattacctgttaatgtagcatttgagctttgggctaagcgcaacattaaaccagtacc |
| | | | | | | | agaggtgaaaatactcaataatttgggtgtggacattgctgctaatactgtgatctgggactacaaaagagatgctcc |
| | | | | | | | agcacatatatctactattggtgtttgttctatgactgacatagccaagaaaccaactgaaacgatttgtgcaccactc |
| | | | | | | | actgtcttttttgatggtagagttgatggtcaagtagacttatttagaaatgcccgtaatggtgttcttattacagaag |
| | | | | | | | gtagtgttaaaggtttacaaccatctgtaggtcccaaacaagctagtcttaatggagtcacattaattggagaagccgt |
| | | | | | | | aaaaacacagttcaattattataagaaagttgatggtgttgtccaacaattacctgaaacttactttactcagagtaga |
| | | | | | | | aatttacaagaatttaaacccaggagtcaaatggaaattgatttcttagaattagctatggatgaattcattgaacggt |
| | | | | | | | ataaattagaaggctatgccttcgaacatatcgtttatggagattgacaaggcgttccaattaacaccaatagcagtcc |
| | | | | | | | agatgaccaaattggctactaccgaagagctaccagacgaattcgtggtggtgacggtaaaatgaaagatctcagtcc |
| | | | | | | | aagatggtatttctactacctaggaactgggccagaagctggacttccctatggtgctaacaaagacggcatcatat |
| | | | | | | | gggttgcaactgagggagccttgaatacaccaaaagatcacattggcacccgcaatccttaaaactgacgttcgtg |
| | | | | | | | ttgttttagatttcatctaaccggataaactaaa |
| 80 | sarbe- | sense_5p_SARS2 | sense | L | DNA or | 1762 | attaaaggtttataccttcccaggtaacaaaccaaccaactttcgatctcttgtagatctgttctctaaccggatttt |
| | covirus | GL_N250_us_ | | | RNA | | aaaatctgtgtggctgtcactcggctgcatgcttagtgcactcacgcagtataattaataactaattactgtcgttgac |
| | | ATG | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattgacaaggcgttccaattaacaccaatagcagtccaga |
| | | | | | | | tgaccaaattggctactaccgaagagctaccagacgaattcgtggtggtgacggtaaaatgaaagatctcagtccaaga |
| | | | | | | | tggtatttctactacctaggaactgggccagaagctggacttccctatggtgctaacaaagacggcatcatatgggtt |
| | | | | | | | gcaactgagggagccttgaatacaccaaaagatcacattggcacccgcaatccttaaaaccaccatggtgacgttc |
| | | | | | | | gtgttgtttaagatttcatctaaccggataaactacaa |
| 81 | sarbe- | sense_5p_SARS2 | sense | L | DNA or | 1501 | attaaaggtttataccttcccaggtaacaaaccaaccaactttcgatctcttgtagatctgttctctaaccggatttta |
| | covirus | GL_PolII_ASA_ | | | RNA | | aaatctgtgtggctgtcactcggctgcatgcttagtgcactcacgcagtataattaataactaattactgtcgttgaca |
| | | ASD | | | | | ggacacgagtaactcgtctatcttctgcaggctgcttacggtttcgtccgtgttgcagccgatcatcagcacatctagg |
| | | | | | | | tttcgtccgggtgtgtgcgaaagcaaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgtttttcatttcatctaaccgg |
| | | | | | | | ataaactaaa |
| 82 | sarbe- | sense_5p_SARS2 | sense | L | DNA or | 1510 | attaaaggtttataccttcccaggtaacaaaccaaccaactttcgatctcttgtagatctgttctctaaccggatttta |
| | covirus | GL_hp_ATG2 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtat |
| | | | | | | | aaattagaaggctatgccttcgaacatatcgtttatggagatttttgacattttagtttatccggttagtttagatttc |
| | | | | | | | atctaaccggataaactaaa |
| 83 | sarbe- | sense_5p_SARS2 | sense | L | DNA or | 1526 | attaaaggtttataccttcccaggtaacaaaccaaccaactttcgatctcttgtagatctgttctctaaccggatttta |
| | covirus | GL_hp_ATG3 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacattttagtttatccggttagatgaaatccgt |
| | | | | | | | gttgttttagatttcatctaaccggataaactaaa |
| 84 | sarbe- | sense_5p_SARS2 | sense | L | DNA or | 1495 | attaaaggtttataccttcccaggtaacaaaccaaccaactttcgatctcttgtagatctgttctctaaccggatttta |
| | covirus | GL_ | | | RNA | | aaatctgtgtggctgtcactcggctgcatgcttagtgcactcacgcagtataattaataactaattactgtcgttgaca |
| | | nonactivatable | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaaaac |
| | | | | | | | taaa |
| 85 | sarbe- | sense_5p_SARS2 | sense | L | DNA or | 1501 | attaaaggtttataccttcccaggtaacaaaccaaccaactttcgatctcttgtagatctgttctctaaccggatttta |
| | covirus | GL_nsp1_124 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttgctgccaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | tgcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaccgg |
| | | | | | | | ataaactaaa |
| 86 | sarbe- | sense_5p_SARS2 | sense | L | DNA or | 1501 | attaaaggtttataccttcccaggtaacaaaccaaccaactttcgatctcttgtagatctgttctctaaccggatttt |
| | covirus | GL_nsp1_124_ | | | RNA | | aaaatctgtgtggctgtcactcggctgcatgcttagtgcactcacgcagtataattaataactaattactgtcgttgac |
| | | 164 | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttgctgccaacggtaataaaggagctggtggccatagttacggcgccgatctaaagtcatttgacttaggcg |
| | | | | | | | acgagcttggcactgatccttatgaagattttcaagaaaactggaacactgccgctagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaccgg |
| | | | | | | | ataaactaaa |
| 87 | sarbe- | sense_5p_SARS2 | sense | L | DNA or | 1502 | attaaaggtttataccttcccaggtaacaaaccaaccaactttcgatctcttgtagatctgttctctaaccggatttt |
| | covirus | GL_nsp1_164 | | | RNA | | aaaatctgtgtggctgtcactcggctgcatgcttagtgcactcacgcagtataattaataactaattactgtcgttgac |
| | | | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactgccgctagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaaccg |
| | | | | | | | gataaactaaa |
| 88 | sarbe- | sense_5p_SARS2 | sense | L | DNA or | 1530 | attaaaggtttataccttcccaggtaacaaaccaaccaactttcgatctcttgtagatctgttctctaaccggatttta |
| | covirus | GL_uATG_ | | | RNA | | aaatctgtgtggctgtcactcggctgcatgcttagtgcactcacgcagtataattaataactaattactgtcgttgac |
| | | NDegron1 | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | tctcatgcgacagggtgaagtaccagtttctatcattaataacactgtttacacaaaagttgatggtgttgatgtagaa |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgaccaccatgcggggcgacctggccctcctgaag |
| | | | | | | | ctgctcgacctgcggtttcatctaaccggataaactaaa |
| 89 | sarbe- | sense_5p_SARS2 | sense | L | DNA or | 1533 | attaaaggtttataccttcccaggtaacaaaccaaccaactttcgatctcttgtagatctgttctctaaccggatttta |
| | covirus | GL_uATG_ | | | RNA | | aaatctgtgtggctgtcactcggctgcatgcttagtgcactcacgcagtataattaataactaattactgtcgttgaca |
| | | NDegron2 | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaaaaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | tgcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgaccaccatgaagatcggcctgggcaggcagaag |
| | | | | | | | ccccctaaagccaccaagtttcatctaaccggataaactaaa |
| 90 | sarbe- | sense_5p_SARS2 | sense | L | DNA or | 1492 | attaaaggtttataccttcccaggtaacaaaccaaccaactttcgatctcttgtagatctgttctctaaccggatttta |
| | covirus | GL_uATG_ | | | RNA | | aaatctgtgtggctgtcactcggctgcatgcttagtgcactcacgcagtataattaataactaattactgtcgttgaca |
| | | overlap | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgaccaccatgtttcatctaaccggataaacta |
| | | | | | | | aaa |
| 91 | sarbe- | sense_5p_SARS2 | sense | L | DNA or | 1210 | attaaaggtttataccttcccaggtaacaaaccaaccaactttcgatctcttgtagatctgttctctaaccggatttta |
| | covirus | GL_A300_locA | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgaggtccctcatgtgggcgaaat |
| | | | | | | | accagtggcttaccgcaaggttcttcttcgtaagaacggtaataaaggagctggtggccatagttacggcgccgat |
| | | | | | | | ctaaagtcatttgacttaggcgacgagcttggcactgatccttatgaagattttcaagaaaactggaacactaaacat |
| | | | | | | | agcagtggtgttacccgtgaactcatgcgacagggtgaagtaccagtttctatcattaataacactgtttacacaaaa |
| | | | | | | | gttgatggtgttgatgtagaattgtttgaaaataaaacaacattacctgttaatgtagcatttgagctttgggctaagc |
| | | | | | | | gcaacattaaaccagtaccagaggtgaaaatactcaataatttgggtgtggacattgctgctaatactgtgatctggga |
| | | | | | | | ctacaaaagagatgctccagcacatatatctactattggtgtttgttctatgactgacatagccaagaaaccaactgaa |
| | | | | | | | acgatttgtgcaccactcactgtcttttttgatggtagagttgatggtcaagtagacttatttagaaatgcccgtaatg |
| | | | | | | | gttcttattacagaaggtagtgttaaaggtttacaaccatctgtaggtcccaaacaagctagtcttaatggagtcacat |
| | | | | | | | tgtaattggagaagccgtaaaaacacagttcaattattataagaaagttgatggtgttgtccaacaattacctgaaact |
| | | | | | | | tactttactcagagtagaaatttacaagaatttaaacccaggagtcaaatggaaattgatttcttagaattagctatgg |
| | | | | | | | atgaattcattgaacggtataaattagaaggctatgccttcgaacatatcgtttatggagatttttgacgttcgtgtt |
| | | | | | | | gttttagatttcatctaaccggataaactaaa |
| 92 | sarbe- | sense_5p_SARS2 | sense | L | DNA or | 1201 | attaaaggtttataccttcccaggtaacaaaccaaccaactttcgatctcttgtagatctgttctctaaccggatttt |
| | covirus | GL_A300_locB | | | RNA | | aaaatctgtgtggctgtcactcggctgcatgcttagtgcactcacgcagtataattaataactaattactgtcgttgac |
| | | | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgttaatgtagcatttgagctttgggctaagcgcaacattaaa |
| | | | | | | | ccagtaccagaggtgaaaatactcaataatttgggtgtggacattgctgctaatactgtgatctgggactacaaaag |
| | | | | | | | agatgctccagcacatatatctactattggtgtttgttctatgactgacatagccaagaaaccaactgaaacgatttgt |
| | | | | | | | gcaccactcactgtcttttttgatggtagagttgatggtcaagtagacttatttagaaatgcccgtaatggtgttctta |
| | | | | | | | ttacagaaggtagtgttaaaggtttacaaccatctgtaggtcccaaacaagctagtcttaatggagtcacattaattgg |
| | | | | | | | agaagccgtaaaaacacagttcaattattataagaaagttgatggtgttgtccaacaattacctgaaacttactttact |
| | | | | | | | cagagtagaaatttacaagaatttaaacccaggagtcaaatggaaattgatttcttagaattagctatggatgaattca |
| | | | | | | | ttgaacggtataaattagaaggctatgccttcgaacatatcgtttatggagatttttgacgttcgtgttgttttagatt |
| | | | | | | | tcatctaaccggataaactaaa |
| 93 | sarbe- | sense_5p_SARS2 | sense | L | DNA or | 1201 | attaaaggtttataccttcccaggtaacaaaccaaccaactttcgatctcttgtagatctgttctctaaccggatttt |
| | covirus | GL_A300_locC | | | RNA | | aaaatctgtgtggctgtcactcggctgcatgcttagtgcactcacgcagtataattaataactaattactgtcgttgac |
| | | | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctttacaaccatctgtaggtcccaaacaagctagtcttaatggagtcacattaat |
| | | | | | | | tggagaagccgtaaaaacacagttcaattattataagaaagttgatggtgttgtccaacaattacctgaaacttacttt |
| | | | | | | | actcagagtagaaatttacaagaatttaaacccaggagtcaaatggaaattgatttcttagaattagctatggatgaat |
| | | | | | | | tcattgaacggtataaattagaaggctatgccttcgaacatatcgtttatggagatttttgacgttcgtgttgtttta |
| | | | | | | | gatttcatctaaccggataaactaaa |
| 94 | sarbe- | sense_5p_SARS2 | sense | L | DNA or | 1231 | attaaaggtttataccttcccaggtaacaaaccaaccaactttcgatctcttgtagatctgttctctaaccggatttt |
| | covirus | GL_A300_locD | | | RNA | | aaaatctgtgtggctgtcactcggctgcatgcttagtgcactcacgcagtataattaataactaattactgtcgttga |
| | | | | | | | caggacacgagtaactcgtctatcttctgcaggctgcttacggtttcgtccgtgttgcagccgatcatcagcacatcta |
| | | | | | | | ggtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggttgacgttcgtgttgttttagatttcatctaaccggataaactaaa |
| 95 | sarbe- | sense_5p_SARS2 | sense | L | DNA or | 1471 | attaaaggtttatagatctcttgtagatctgttctctaaccggattttaaaatctgtgtggctgtcactcggctgcat |
| | covirus | GL_A30_loc01 | | | RNA | | gcttagtgcactcacgcagtataattaataactaattactgtcgttgacaggacacgagtaactcgtctatcttctgc |
| | | | | | | | aggctgcttacggtttcgtccgtgttgcagccgatcatcagcacatctaggtttcgtccgggtgtgaccgaaaggtaa |
| | | | | | | | gatggagagccttgtccctggtttcaacgagaaaacacacgtccaactcagtttgcctgttttacaggttcgcgacgtg |
| | | | | | | | ctcgtacgtggctttggagactccgtggaggaggtcttatcagaggcacgtcaacatcttaaagatggcacttgtggct |
| | | | | | | | tagtagaagttgaaaaaggcgttttgcctcaacttgaacagccctatgtgttcatcaaacgttcggatgctcgaactgc |
| | | | | | | | acctcatggtcatgttatggttgagctggtagcagaactcgaaggcattcagtacggtcgtagtggtgagacacttg |
| | | | | | | | gtgtccttgtccctcatgtgggcgaaataccagtggcttaccgcaaggttcttcttcgtaagaacggtaataaaggag |
| | | | | | | | ctggtggccatagttacggcgccgatctaaagtcatttgacttaggcgacgagcttggcactgatccttatgaagatt |
| | | | | | | | ttcaagaaaactggaacactaaacatagcagtggtgttacccgtgaactcatgcgacagggtgaagtaccagtttct |
| | | | | | | | atcattaataacactgtttacacaaaagttgatggtgttgatgtagaattgtttgaaaataaaacaacattacctgtt |
| | | | | | | | aatgtagcatttgagctttgggctaagcgcaacattaaaccagtaccagaggtgaaaatactcaataatttgggtgtgg |
| | | | | | | | acattgctgctaatactgtgatctgggactacaaaagagatgctccagcacatatatctactattggtgtttgttctat |
| | | | | | | | gactgacatagccaagaaaccaactgaaacgatttgtgcaccactcactgtcttttttgatggtagagttgatggtcaa |
| | | | | | | | gtagacttatttagaaatgcccgtaatggtgttcttattacagaaggtagtgttaaaggtttacaaccatctgtaggt |
| | | | | | | | cccaaacaagctagtcttaatggagtcacattaattggagaagccgtaaaaacacagttcaattattataagaaagttg |
| | | | | | | | atggtgttgtccaacaattacctgaaacttactttactcagagtagaaatttacaagaatttaaacccaggagtcaaat |
| | | | | | | | ggaaattgatttcttagaattagctatggatgaattcattgaacggtataaattagaaggctatgccttcgaacatatc |
| | | | | | | | gtttatggagatttttgacgttcgtgttgttttagatttcatctaaccggataaactaaa |
| 96 | sarbe- | sense_5p_SARS2 | sense | L | DNA or | 1471 | attaaaggtttataccttcccaggtaacaaaccaaccaactttcgatctcttgtagatctgttctctaaccggatttt |
| | covirus | GL_A30_loc02 | | | RNA | | aaaatctgcactcacgcagtataattaataactaattactgtcgttgacaggacacgagtaactcgtctatcttctgca |
| | | | | | | | ggctgcttacggtttcgtccgtgttgcagccgatcatcagcacatctaggtttcgtccgggtgtgaccgaaaggtaaga |
| | | | | | | | tggagagccttgtccctggtttcaacgagaaaacacacgtccaactcagtttgcctgttttacaggttcgcgacgtgct |
| | | | | | | | cgtacgtggctttggagactccgtggaggaggtcttatcagaggcacgtcaacatcttaaagatggcacttgtggctt |
| | | | | | | | agtagaagttgaaaaaggcgttttgcctcaacttgaacagccctatgtgttcatcaaacgttcggatgctcgaactgc |
| | | | | | | | acctcatggtcatgttatggttgagctggtagcagaactcgaaggcattcagtacggtcgtagtggtgagacacttg |
| | | | | | | | gtgtccttgtccctcatgtgggcgaaataccagtggcttaccgcaaggttcttcttcgtaagaacggtaataaaggag |
| | | | | | | | ctggtggccatagttacggcgccgatctaaagtcatttgacttaggcgacgagcttggcactgatccttatgaagatt |
| | | | | | | | ttcaagaaaactggaacactaaacatagcagtggtgttacccgtgaactcatgcgacagggtgaagtaccagtttct |
| | | | | | | | atcattaataacactgtttacacaaaagttgatggtgttgatgtagaattgtttgaaaataaaacaacattacctgtt |
| | | | | | | | aatgtagcatttgagctttgggctaagcgcaacattaaaccagtaccagaggtgaaaatactcaataatttgggtgtgg |
| | | | | | | | acattgctgctaatactgtgatctgggactacaaaagagatgctccagcacatatatctactattggtgtttgttctat |
| | | | | | | | gactgacatagccaagaaaccaactgaaacgatttgtgcaccactcactgtcttttttgatggtagagttgatggtcaa |
| | | | | | | | gtagacttatttagaaatgcccgtaatggtgttcttattacagaaggtagtgttaaaggtttacaaccatctgtaggtc |
| | | | | | | | ccaaacaagctagtcttaatggagtcacattaattggagaagccgtaaaaacacagttcaattattataagaaagttga |
| | | | | | | | tggtgttgtccaacaattacctgaaacttactttactcagagtagaaatttacaagaatttaaacccaggagtcaaatg |
| | | | | | | | gaaattgatttcttagaattagctatggatgaattcattgaacggtataaattagaaggctatgccttcgaacatatc |
| | | | | | | | gtttatggagatttttgacgttcgtgttgttttagatttcatctaaccggataaactaaa |
| 97 | sarbe- | sense_5p_SARS2 | sense | L | DNA or | 1471 | attaaaggtttataccttcccaggtaacaaaccaaccaactttcgatctcttgtagatctgttctctaaccggatttt |
| | covirus | GL_A30_loc03 | | | RNA | | aaaatctgtgtggctgtcactcggctgcatgcttagtgcactcacgcagtatacacgagtaactcgtctatcttctgca |
| | | | | | | | ggctgcttacggtttcgtccgtgttgcagccgatcatcagcacatctaggtttcgtccgggtgtgaccgaaaggtaaga |
| | | | | | | | tggagagccttgtccctggtttcaacgagaaaacacacgtccaactcagtttgcctgttttacaggttcgcgacgtgct |
| | | | | | | | cgtacgtggctttggagactccgtggaggaggtcttatcagaggcacgtcaacatcttaaagatggcacttgtggctt |
| | | | | | | | agtagaagttgaaaaaggcgttttgcctcaacttgaacagccctatgtgttcatcaaacgttcggatgctcgaactgc |
| | | | | | | | acctcatggtcatgttatggttgagctggtagcagaactcgaaggcattcagtacggtcgtagtggtgagacacttg |
| | | | | | | | gtgtccttgtccctcatgtgggcgaaataccagtggcttaccgcaaggttcttcttcgtaagaacggtaataaaggag |
| | | | | | | | ctggtggccatagttacggcgccgatctaaagtcatttgacttaggcgacgagcttggcactgatccttatgaagatt |
| | | | | | | | ttcaagaaaactggaacactaaacatagcagtggtgttacccgtgaactcatgcgacagggtgaagtaccagtttct |
| | | | | | | | atcattaataacactgtttacacaaaagttgatggtgttgatgtagaattgtttgaaaataaaacaacattacctgtt |
| | | | | | | | aatgtagcatttgagctttgggctaagcgcaacattaaaccagtaccagaggtgaaaatactcaataatttgggtgtgg |
| | | | | | | | acattgctgctaatactgtgatctgggactacaaaagagatgctccagcacatatatctactattggtgtttgttctat |
| | | | | | | | gactgacatagccaagaaaccaactgaaacgatttgtgcaccactcactgtcttttttgatggtagagttgatggtcaa |
| | | | | | | | gtagacttatttagaaatgcccgtaatggtgttcttattacagaaggtagtgttaaaggtttacaaccatctgtaggtc |
| | | | | | | | ccaaacaagctagtcttaatggagtcacattaattggagaagccgtaaaaacacagttcaattattataagaaagttga |
| | | | | | | | tggtgttgtccaacaattacctgaaacttactttactcagagtagaaatttacaagaatttaaacccaggagtcaaatg |
| | | | | | | | gaaattgatttcttagaattagctatggatgaattcattgaacggtataaattagaaggctatgccttcgaacatatcg |
| | | | | | | | tttatggagatttttgacgttcgtgttgttttagatttcatctaaccggataaactaaa |
| 98 | sarbe- | sense_5p_SARS2 | sense | L | DNA or | 1471 | attaaaggtttataccttcccaggtaacaaaccaaccaactttcgatctcttgtagatctgttctctaaccggatttt |
| | covirus | GL_A30_loc04 | | | RNA | | aaaatctgtgtggctgtcactcggctgcatgcttagtgcactcacgcagtataattaataactaattactgtcgttga |
| | | | | | | | caggacacgagtaactcgtctatcttgcagccgatcatcagcacatctaggtttcgtccgggtgtgaccgaaaggtaag |
| | | | | | | | atggagagccttgtccctggtttcaacgagaaaacacacgtccaactcagtttgcctgttttacaggttcgcgacgtgc |
| | | | | | | | tcgtacgtggctttggagactccgtggaggaggtcttatcagaggcacgtcaacatcttaaagatggcacttgtggct |
| | | | | | | | tagtagaagttgaaaaaggcgttttgcctcaacttgaacagccctatgtgttcatcaaacgttcggatgctcgaactg |
| | | | | | | | cacctcatggtcatgttatggttgagctggtagcagaactcgaaggcattcagtacggtcgtagtggtgagacactt |
| | | | | | | | ggtgtccttgtccctcatgtgggcgaaataccagtggcttaccgcaaggttcttcttcgtaagaacggtaataaagga |
| | | | | | | | gctggtggccatagttacggcgccgatctaaagtcatttgacttaggcgacgagcttggcactgatccttatgaaga |
| | | | | | | | ttttcaagaaaactggaacactaaacatagcagtggtgttacccgtgaactcatgcgacagggtgaagtaccagttt |
| | | | | | | | ctatcattaataacactgtttacacaaaagttgatggtgttgatgtagaattgtttgaaaataaaacaacattacctgt |
| | | | | | | | taatgtagcatttgagctttgggctaagcgcaacattaaaccagtaccagaggtgaaaatactcaataatttgggtgtg |
| | | | | | | | gacattgctgctaatactgtgatctgggactacaaaagagatgctccagcacatatatctactattggtgtttgttcta |
| | | | | | | | tgactgacatagccaagaaaccaactgaaacgatttgtgcaccactcactgtcttttttgatggtagagttgatggtca |
| | | | | | | | agtagacttatttagaaatgcccgtaatggtgttcttattacagaaggtagtgttaaaggtttacaaccatctgtaggt |
| | | | | | | | cccaaacaagctagtcttaatggagtcacattaattggagaagccgtaaaaacacagttcaattattataagaaagttg |
| | | | | | | | atggtgttgtccaacaattacctgaaacttactttactcagagtagaaatttacaagaatttaaacccaggagtcaaat |
| | | | | | | | ggaaattgatttcttagaattagctatggatgaattcattgaacggtataaattagaaggctatgccttcgaacatatc |
| | | | | | | | gtttatggagatttttgacgttcgtgttgttttagatttcatctaaccggataaactaaa |
| 99 | sarbe- | sense_5p_SARS2 | sense | L | DNA or | 1471 | attaaaggtttataccttcccaggtaacaaaccaaccaactttcgatctcttgtagatctgttctctaaccggatttta |
| | covirus | GL_A30_loc05 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcaggtaagat |
| | | | | | | | ggagagccttgtccctggtttcaacgagaaaacacacgtccaactcagtttgcctgttttacaggttcgcgacgtgctc |
| | | | | | | | gtacgtggctttggagactccgtggaggaggtcttatcagaggcacgtcaacatcttaaagatggcacttgtggctt |
| | | | | | | | agtagaagttgaaaaaggcgttttgcctcaacttgaacagccctatgtgttcatcaaacgttcggatgctcgaactgc |
| | | | | | | | acctcatggtcatgttatggttgagctggtagcagaactcgaaggcattcagtacggtcgtagtggtgagacacttg |
| | | | | | | | gtgtccttgtccctcatgtgggcgaaataccagtggcttaccgcaaggttcttcttcgtaagaacggtaataaaggag |
| | | | | | | | ctggtggccatagttacggcgccgatctaaagtcatttgacttaggcgacgagcttggcactgatccttatgaagatt |
| | | | | | | | ttcaagaaaactggaacactaaacatagcagtggtgttacccgtgaactcatgcgacagggtgaagtaccagtttct |
| | | | | | | | atcattaataacactgtttacacaaaagttgatggtgttgatgtagaattgtttgaaaataaaacaacattacctgtta |
| | | | | | | | gtagcatttgagctttgggctaagcgcaacattaaaccagtaccagaggtgaaaatactcaataatttgggtgtgga |
| | | | | | | | atcattgctgctaatactgtgatctgggactacaaaagagatgctccagcacatatatctactattggtgtttgttcta |
| | | | | | | | tgactgacatagccaagaaaccaactgaaacgatttgtgcaccactcactgtcttttttgatggtagagttgatggtca |
| | | | | | | | agtagacttatttagaaatgcccgtaatggtgttcttattacagaaggtagtgttaaaggtttacaaccatctgtaggt |
| | | | | | | | cccaaacaagctagtcttaatggagtcacattaattggagaagccgtaaaaacacagttcaattattataagaaagttg |
| | | | | | | | atggtgttgtccaacaattacctgaaacttactttactcagagtagaaatttacaagaatttaaacccaggagtcaaat |
| | | | | | | | ggaaattgatttcttagaattagctatggatgaattcattgaacggtataaattagaaggctatgccttcgaacatatc |
| | | | | | | | gtttatggagatttttgacgttcgtgttgttttagatttcatctaaccggataaactaaa |
| 100 | sarbe- | sense_5p_SARS2 | sense | L | DNA or | 1471 | attaaaggtttataccttcccaggtaacaaaccaaccaactttcgatctcttgtagatctgttctctaaccggatttta |
| | covirus | GL_A30_loc06 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatgacacacgtccaactcagtttgcctgttttacaggttcgcgacgtgctc |
| | | | | | | | gtacgtggctttggagactccgtggaggaggtcttatcagaggcacgtcaacatcttaaagatggcacttgtggctt |
| | | | | | | | agtagaagttgaaaaaggcgttttgcctcaacttgaacagccctatgtgttcatcaaacgttcggatgctcgaactgc |
| | | | | | | | acctcatggtcatgttatggttgagctggtagcagaactcgaaggcattcagtacggtcgtagtggtgagacacttg |
| | | | | | | | gtgtccttgtccctcatgtgggcgaaataccagtggcttaccgcaaggttcttcttcgtaagaacggtaataaaggag |
| | | | | | | | ctggtggccatagttacggcgccgatctaaagtcatttgacttaggcgacgagcttggcactgatccttatgaagatt |
| | | | | | | | ttcaagaaaactggaacactaaacatagcagtggtgttacccgtgaactcatgcgacagggtgaagtaccagtttct |
| | | | | | | | atcattaataacactgtttacacaaaagttgatggtgttgatgtagaattgtttgaaaaaaaacaacattacctgttaa |
| | | | | | | | tgtagcatttgagctttgggctaagcgcaacattaaaccagtaccagaggtgaaaatactcaataatttgggtgtgga |
| | | | | | | | cattgctgctaatactgtgatctgggactacaaaagagatgctccagcacatatatctactattggtgtttgttctatg |
| | | | | | | | actgacatagccaagaaaccaactgaaacgatttgtgcaccactcactgtcttttttgatggtagagttgatggtcaag |
| | | | | | | | tagacttatttagaaatgcccgtaatggtgttcttattacagaaggtagtgttaaaggtttacaaccatctgtaggtcc |
| | | | | | | | caaacaagctagtcttaatggagtcacattaattggagaagccgtaaaaacacagttcaattattataagaaagttgat |
| | | | | | | | ggtgttgtccaacaattacctgaaacttactttactcagagtagaaatttacaagaatttaaacccaggagtcaaatgg |
| | | | | | | | aaattgatttcttagaattagctatggatgaattcattgaacggtataaattagaaggctatgccttcgaacatatcgt |
| | | | | | | | ttatggagatttttgacgttcgtgttgttttagatttcatctaaccggataaactaaa |
| 101 | sarbe- | sense_5p_SARS2 | sense | L | DNA or | 1471 | attaaaggtttataccttcccaggtaacaaaccaaccaactttcgatctcttgtagatctgttctctaaccggatttta |
| | covirus | GL_A30_loc07 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtcgacgtgc |
| | | | | | | | tcgtacgtggctttggagactccgtggaggaggtcttatcagaggcacgtcaacatcttaaagatggcacttgtggc |
| | | | | | | | ttagtagaagttgaaaaaggcgttttgcctcaacttgaacagccctatgtgttcatcaaacgttcggatgctcgaactg |
| | | | | | | | cacctcatggtcatgttatggttgagctggtagcagaactcgaaggcattcagtacggtcgtagtggtgagacactt |
| | | | | | | | ggtgtccttgtccctcatgtgggcgaaataccagtggcttaccgcaaggttcttcttcgtaagaacggtaataaagga |
| | | | | | | | gctggtggccatagttacggcgccgatctaaagtcatttgacttaggcgacgagcttggcactgatccttatgaaga |
| | | | | | | | ttttcaagaaaactggaacactaaacatagcagtggtgttacccgtgaactcatgcgacagggtgaagtaccagttt |
| | | | | | | | ctatcattaataacactgtttacacaaaagttgatggtgttgatgtagaattgtttgaaaataaaacaacattacctgt |
| | | | | | | | taatgtagcatttgagctttgggctaagcgcaacattaaaccagtaccagaggtgaaaatactcaataatttgggtgtg |
| | | | | | | | gacattgctgctaatactgtgatctgggactacaaaagagatgctccagcacatatatctactattggtgtttgttcta |
| | | | | | | | tgactgacatagccaagaaaccaactgaaacgatttgtgcaccactcactgtcttttttgatggtagagttgatggtca |
| | | | | | | | agtagacttatttagaaatgcccgtaatggtgttcttattacagaaggtagtgttaaaggtttacaaccatctgtaggt |
| | | | | | | | cccaaacaagctagtcttaatggagtcacattaattggagaagccgtaaaaacacagttcaattattataagaaagttg |
| | | | | | | | atggtgttgtccaacaattacctgaaacttactttactcagagtagaaatttacaagaatttaaacccaggagtcaaat |
| | | | | | | | ggaaattgatttcttagaattagctatggatgaattcattgaacggtataaattagaaggctatgccttcgaacatatc |
| | | | | | | | gtttatggagatttttgacgttcgtgttgttttagatttcatctaaccggataaactaaa |
| 102 | sarbe- | sense_5p_SARS2 | sense | L | DNA or | 1471 | attaaaggtttataccttcccaggtaacaaaccaaccaactttcgatctcttgtagatctgttctctaaccggatttta |
| | covirus | GL_A30_loc08 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgttcagaggcacgtcaacatcttaaagatggcacttgtggctt |
| | | | | | | | agtagaagttgaaaaaggcgttttgcctcaacttgaacagccctatgtgttcatcaaacgttcggatgctcgaactgc |
| | | | | | | | acctcatggtcatgttatggttgagctggtagcagaactcgaaggcattcagtacggtcgtagtggtgagacacttg |
| | | | | | | | gtgtccttgtccctcatgtgggcgaaataccagtggcttaccgcaaggttcttcttcgtaagaacggtaataaaggag |
| | | | | | | | ctggtggccatagttacggcgccgatctaaagtcatttgacttaggcgacgagcttggcactgatccttatgaagatt |
| | | | | | | | ttcaagaaaactggaacactaaacatagcagtggtgttacccgtgaactcatgcgacagggtgaagtaccagtttct |
| | | | | | | | atcattaataacactgtttacacaaaagttgatggtgttgatgtagaattgtttgaaaataaaacaacattacctgtt |
| | | | | | | | aatgtagcatttgagctttgggctaagcgcaacattaaaccagtaccagaggtgaaaatactcaataatttgggtgtgg |
| | | | | | | | acattgctgctaatactgtgatctgggactacaaaagagatgctccagcacatatatctactattggtgtttgttctat |
| | | | | | | | gactgacatagccaagaaaccaactgaaacgatttgtgcaccactcactgtcttttttgatggtagagttgatggtcaa |
| | | | | | | | gtagacttatttagaaatgcccgtaatggtgttcttattacagaaggtagtgttaaaggtttacaaccatctgtaggtc |
| | | | | | | | ccaaacaagctagtcttaatggagtcacattaattggagaagccgtaaaaacacagttcaattattataagaaagttga |
| | | | | | | | tggtgttgtccaacaattacctgaaacttactttactcagagtagaaatttacaagaatttaaacccaggagtcaaatg |
| | | | | | | | gaaattgatttcttagaattagctatggatgaattcattgaacggtataaattagaaggctatgccttcgaacatatcg |
| | | | | | | | tttatggagatttttgacgttcgtgttgttttagatttcatctaaccggataaactaaa |
| 103 | sarbe- | sense_5p_SARS2 | sense | L | DNA or | 1471 | attaaaggtttataccttcccaggtaacaaaccaaccaactttcgatctcttgtagatctgttctctaaccggatttta |
| | covirus | GL_A30_loc09 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaaaaaggcgttttgcctcaacttgaacagccctatgtgttcatcaaacgttcggatgctcgaactgc |
| | | | | | | | acctcatggtcatgttatggttgagctggtagcagaactcgaaggcattcagtacggtcgtagtggtgagacacttg |
| | | | | | | | gtgtccttgtccctcatgtgggcgaaataccagtggcttaccgcaaggttcttcttcgtaagaacggtaataaaggag |
| | | | | | | | ctggtggccatagttacggcgccgatctaaagtcatttgacttaggcgacgagcttggcactgatccttatgaagatt |
| | | | | | | | ttcaagaaaactggaacactaaacatagcagtggtgttacccgtgaactcatgcgacagggtgaagtaccagtttct |
| | | | | | | | atcattaataacactgtttacacaaaagttgatggtgttgatgtagaattgtttgaaaaaaaacaacattacctgttaa |
| | | | | | | | tgtagcatttgagctttgggctaagcgcaacattaaaccagtaccagaggtgaaaatactcaataatttgggtgtgga |
| | | | | | | | cattgctgctaatactgtgatctgggactacaaaagagatgctccagcacatatatctactattggtgtttgttctatg |
| | | | | | | | actgacatagccaagaaaccaactgaaacgatttgtgcaccactcactgtcttttttgatggtagagttgatggtcaag |
| | | | | | | | tagacttatttagaaatgcccgtaatggtgttcttattacagaaggtagtgttaaaggtttacaaccatctgtaggtcc |
| | | | | | | | caaacaagctagtcttaatggagtcacattaattggagaagccgtaaaaacacagttcaattattataagaaagttgat |
| | | | | | | | ggtgttgtccaacaattacctgaaacttactttactcagagtagaaatttacaagaatttaaacccaggagtcaaatgg |
| | | | | | | | aaattgatttcttagaattagctatggatgaattcattgaacggtataaattagaaggctatgccttcgaacatatcgt |
| | | | | | | | ttatggagatttttgacgttcgtgttgttttagatttcatctaaccggataaactaaa |
| 104 | sarbe- | sense_5p_ | sense | L | DNA or | 1513 | attaaaggtttataccttcccaggtaacaaaccaaccaactttcgatctcttgtagatctgttctctaaacgaacttta |
| | covirus | SARS2_ATG_HP15 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttctcccattttagtgacgttcgtgttgttttagatt |
| | | | | | | | tcatctaaacgaacaaactaaa |
| 105 | sarbe- | sense_5p_ | sense | L | DNA or | 1522 | attaaaggtttataccttcccaggtaacaaaccaaccaactttcgatctcttgtagatctgttctctaaacgaacttta |
| | covirus | SARS2_ATG_HP30 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttctttgactcccattttagttttgacgttcgtgttg |
| | | | | | | | ttttagatttcatctaaacgaacaaactaaa |
| 106 | sarbe- | sense_5p_ | sense | L | DNA or | 1529 | attaaaggtttataccttcccaggtaacaaaccaaccaactttcgatctcttgtagatctgttctctaaacgaacttta |
| | covirus | SARS2_ATG_HP45 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaaaaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttgaactttgactcccattttagtttatcctgacgtt |
| | | | | | | | cgtgttgttttagatttcatctaaacgaacaaactaaa |
| 107 | sarbe- | sense_5p_ | sense | L | DNA or | 1536 | attaaaggtttataccttcccaggtaacaaaccaaccaactttcgatctcttgtagatctgttctctaaacgaacttta |
| | covirus | SARS2_ATG_HP60 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattttcaaacagaactttgactcccattttagtttatcc |
| | | | | | | | gtgacgttcgtgttgttttagatttcatctaaacgaacaaactaaa |
| 108 | sarbe- | sense_5p_ | sense | L | DNA or | 1552 | taaacctttaatctgatgagtccgtgaggacgaaacccggagtcccgggtcattaaaggtttataccttcccaggtaa |
| | covirus | SARS2_GGC_HHRz | | | RNA | | caaaccaaccaactttcgatctcttgtagatctgttctctaaacgaactttaaaatctgtgtggctgtcactcggctgc |
| | | | | | | | atgcttagtgcactcacgcagtataattaataactaattactgtcgttgacaggacacgagtaactcgtctatcttctg |
| | | | | | | | caggctgcttacggtttcgtccgtgttgcagccgatcatcagcacatctaggtttcgtccgggtgtgaccgaaaggtaa |
| | | | | | | | gatggagagccttgtccctggtttcaacgagaaaacacacgtccaactcagtttgcctgttttacaggttcgcgacgt |
| | | | | | | | gctcgtacgtggctttggagactccgtggaggaggtcttatcagaggcacgtcaacatcttaaagatggcacttgtg |
| | | | | | | | gcttagtagaagttgaaaaaggcgttttgcctcaacttgaacagccctatgtgttcatcaaacgttcggatgctcgaa |
| | | | | | | | ctgcacctcatggtcatgttatggttgagctggtagcagaactcgaaggcattcagtacggtcgtagtggtgagaca |
| | | | | | | | cttggtgtccttgtccctcatgtgggcgaaataccagtggcttaccgcaaggttcttcttcgtaagaacggtaataaa |
| | | | | | | | ggagctggtggccatagttacggcgccgatctaaagtcatttgacttaggcgacgagcttggcactgatccttatga |
| | | | | | | | agattttcaagaaaactggaacactaaacatagcagtggtgttacccgtgaactcatgcgacagggtgaagtacca |
| | | | | | | | gtttctatcattaataacactgtttacacaaaagttgatggtgttgatgtagaattgtttgaaaataaaacaacattac |
| | | | | | | | ctgttaatgtagcatttgagctttgggctaagcgcaacattaaaccagtaccagaggtgaaaatactcaataatttggg |
| | | | | | | | tgtggacattgctgctaatactgtgatctgggactacaaaagagatgctccagcacatatatctactattggtgtttgt |
| | | | | | | | tctatgactgacatagccaagaaaccaactgaaacgatttgtgcaccactcactgtcttttttgatggtagagttgatg |
| | | | | | | | agtagacttatttagaaatgcccgtaatggtgttcttattacagaaggtagtgttaaaggtttacaaccatctgtaggt |
| | | | | | | | cgtcaccaaacaagctagtcttaatggagtcacattaattggagaagccgtaaaaacacagttcaattattataagaaa |
| | | | | | | | gttgatggtgttgtccaacaattacctgaaacttactttactcagagtagaaatttacaagaatttaaacccaggagtc |
| | | | | | | | aaatggaaattgatttcttagaattagctatggatgaattcattgaacggtataaattagaaggctatgccttcgaaca |
| | | | | | | | tatcgtttatggagatttttgacgttcgtgttgttttagatttcatctaaacgaacaaactaaa |
| 109 | sarbe- | sense_5p_ | sense | L | DNA or | 1753 | attaaaggtttataccttcccaggtaacaaaccaaccaactttcgatctcttgtagatctgttctctaaacgaacttta |
| | covirus | SARS2_N250 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattgacaaggcgttccaattaacaccaatagcagtccaga |
| | | | | | | | tgaccaaattggctactaccgaagagctaccagacgaattcgtggtggtgacggtaaaatgaaagatctcagtccaaga |
| | | | | | | | tggtatttctactacctaggaactgggccagaagctggacttccctatggtgctaacaaagacggcatcatatgggtt |
| | | | | | | | gcaactgagggagccttgaatacaccaaaagatcacattggcacccgcaatccttaaaactgacgttcgtgttgtttt |
| | | | | | | | agatttcatctaaacgaacaaactaaa |
| 110 | sarbe- | sense_5p_ | sense | L | DNA or | 1771 | attaaaggtttataccttcccaggtaacaaaccaaccaactttcgatctcttgtagatctgttctctaaacgaacttta |
| | covirus | SARS2_N250_HP10 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattgacaaggcgttccaattaacaccaatagcagtccag |
| | | | | | | | atgaccaaattggctactaccgaagagctaccagacgaattcgtggtggtgacggtaaaatgaaagatctcagtccaa |
| | | | | | | | gatggtatttctactacctaggaactgggccagaagctggacttccctatggtgctaacaaagacggcatcatatgggt |
| | | | | | | | tgcaactgagggagccttgaatacaccaacggccgatatcacggccgaagatcacattggcacccgcaatccttaa |
| | | | | | | | aactgacgttcgtgttgttttagatttcatctaaacgaacaaactaaa |
| 111 | sarbe- | sense_5p_ | sense | L | DNA or | 1787 | attaaaggtttataccttcccaggtaacaaaccaaccaactttcgatctcttgtagatctgttctctaaacgaacttta |
| | covirus | SARS2_N250_HP30 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattgacaaggcgttccaattaacaccaatagcagtccaga |
| | | | | | | | tgaccaaattggctactaccgaagagctaccagacgaattcgtggtggtgacggtaaaatgaaagatctcagtccaaga |
| | | | | | | | tggtatttctactacctaggaactgggccagaagctggacttccctatggtgctaacaaagacggcatcatatgggtt |
| | | | | | | | gcaactgagggagccttgaatacaccaagtccaccacggccgatatcacggccgtggtggacaagatcacattg |
| | | | | | | | gcacccgcaatccttaaaactgacgttcgtgttgttttagatttcatctaaacgaacaaactaaa |
| 112 | sarbe- | sense_5p_ | sense | L | DNA or | 1801 | attaaaggtttataccttcccaggtaacaaaccaaccaactttcgatctcttgtagatctgttctctaaacgaacttt |
| | covirus | SARS2_N250_ | | | RNA | | aaaatctgtgtggctgtcactcggctgcatgcttagtgcactcacgcagtataattaataactaattactgtcgttga |
| | | HP50 | | | | | caggacacgagtaactcgtctatcttctgcaggctgcttacggtttcgtccgtgttgcagccgatcatcagcacatct |
| | | | | | | | aggtttcgtccgggtgtgaccgaaaggtaagatggagagccttgtccctggtttcaacgagaaaacacacgtccaact |
| | | | | | | | cagtttgcctgttttacaggttcgcgacgtgctcgtacgtggctttggagactccgtggaggaggtcttatcagaggc |
| | | | | | | | acgtcaacatcttaaagatggcacttgtggcttagtagaagttgaaaaaggcgttttgcctcaacttgaacagccctat |
| | | | | | | | gtgttcatcaaacgttcggatgctcgaactgcacctcatggtcatgttatggttgagctggtagcagaactcgaaggca |
| | | | | | | | ttcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattgacaaggcgttccaattaacaccaatagcagtccaga |
| | | | | | | | tgaccaaattggctactaccgaagagctaccagacgaattcgtggtggtgacggtaaaatgaaagatctcagtccaaga |
| | | | | | | | tggtatttctactacctaggaactgggccagaagctggacttccctatggtgctaacaaagacggcatcatatgggtt |
| | | | | | | | gcaactgagggagccttgaatacaccaaccctgcggtccaccacggccgatatcacggccgtggtggaccgca |
| | | | | | | | gggaagatcacattggcacccgcaatccttaaaactgacgttcgtgttgttttagatttcatctaaacgaacaaactaa |
| | | | | | | | a |
| 113 | sarbe- | sense_5p_ | sense | L | DNA or | 1754 | attaaaggtttataccttcccaggtaacaaaccaaccaactttcgatctcttgtagatctgttctctaaacgaacttt |
| | covirus | SARS2_N250_ | | | RNA | | aaaatctgtgtggctgtcactcggctgcatgcttagtgcactcacgcagtataattaataactaattactgtcgttga |
| | | stop | | | | | caggacacgagtaactcgtctatcttctgcaggctgcttacggtttcgtccgtgttgcagccgatcatcagcacatcta |
| | | | | | | | ggtttcgtccgggtgtgaccgaaaggtaagatggagagccttgtccctggtttcaacgagaaaacacacgtccaactca |
| | | | | | | | gtttgcctgtttaaacaggttcgcgacgtgctcgtacgtggctttggagactccgtggaggaggtcttatcagaggc |
| | | | | | | | acgtcaacatcttaaagatggcacttgtggcttagtagaagttgaaaaaggcgttttgcctcaacttgaacagcccta |
| | | | | | | | tgtgttcatcaaacgttcggatgctcgaactgcacctcatggtcatgttatggttgagctggtagcagaactcgaagg |
| | | | | | | | cattcagtacggtcgtagtggtgagacacttggtgtccttgtccctcatgtgggcgaaataccagtggcttaccgca |
| | | | | | | | aggttcttcttcgtaagaacggtaataaaggagctggtggccatagttacggcgccgatctaaagtcatttgacttag |
| | | | | | | | gcgacgagcttggcactgatccttatgaagattttcaagaaaactggaacactaaacatagcagtggtgttacccgt |
| | | | | | | | gaactcatgcgacagggtgaagtaccagtttctatcattaataacactgtttacacaaaagttgatggtgttgatgtag |
| | | | | | | | aattgtttgaaaataaaacaacattacctgttaatgtagcatttgagctttgggctaagcgcaacattaaaccagtacc |
| | | | | | | | agaggtgaaaatactcaataatttgggtgtggacattgctgctaatactgtgatctgggactacaaaagagatgctcc |
| | | | | | | | agcacatatatctactattggtgtttgttctatgactgacatagccaagaaaccaactgaaacgatttgtgcaccactc |
| | | | | | | | actgtcttttttgatggtagagttgatggtcaagtagacttatttagaaatgcccgtaatggtgttcttattacagaag |
| | | | | | | | gtagtgttaaaggtttacaaccatctgtaggtcccaaacaagctagtcttaatggagtcacattaattggagaagccgt |
| | | | | | | | aaaaacacagttcaattattataagaaagttgatggtgttgtccaacaattacctgaaacttactttactcagagtaga |
| | | | | | | | aatttacaagaatttaaacccaggagtcaaatggaaattgatttcttagaattagctatggatgaattcattgaacggt |
| | | | | | | | ataaattagaaggctatgccttcgaacatatcgtttatggagattgacaaggcgttccaattaacaccaatagcagtcc |
| | | | | | | | agatgaccaaattggctactaccgaagagctaccagacgaattcgtggtggtgacggtaaaatgaaagatctcagtcc |
| | | | | | | | aagatggtatttctactacctaggaactgggccagaagctggacttccctatggtgctaacaaagacggcatcatat |
| | | | | | | | gggttgcaactgagggagccttgaatacaccaaaagatcacattggcacccgcaatccttaaaactgacgttcgtg |
| | | | | | | | ttgttttagatttcatctaaacgaacaaactaaa |
| 114 | sarbe- | sense_5p_ | sense | L | DNA or | 1762 | attaaaggtttataccttcccaggtaacaaaccaaccaactttcgatctcttgtagatctgttctctaaacgaacttta |
| | covirus | SARS2_N250_ | | | RNA | | aaatctgtgtggctgtcactcggctgcatgcttagtgcactcacgcagtataattaataactaattactgtcgttgaca |
| | | us_ATG | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagattgacaaggcgttccaattaacaccaatagcagtccaga |
| | | | | | | | tgaccaaattggctactaccgaagagctaccagacgaattcgtggtggtgacggtaaaatgaaagatctcagtccaaga |
| | | | | | | | tggtatttctactacctaggaactgggccagaagctggacttccctatggtgctaacaaagacggcatcatatgggtt |
| | | | | | | | gcaactgagggagccttgaatacaccaaaagatcacattggcacccgcaatccttaaaaccaccatggtgacgttc |
| | | | | | | | gtgttgtttaagatttcatctaaacgaacaaactacaa |
| 115 | sarbe- | sense_5p_ | sense | L | DNA or | 1501 | attaaaggtttataccttcccaggtaacaaaccaaccaactttcgatctcttgtagatctgttctctaaacgaacttta |
| | covirus | SARS2PolII_ | | | RNA | | aaatctgtgtggctgtcactcggctgcatgcttagtgcactcacgcagtataattaataactaattactgtcgttgaca |
| | | ΔSA_ΔSD | | | | | ggacacgagtaactcgtctatcttctgcaggctgcttacggtttcgtccgtgttgcagccgatcatcagcacatctagg |
| | | | | | | | tttcgtccgggtgtgtgcgaaagcaaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgtttttcatttcatctaaacga |
| | | | | | | | acaaactaaa |
| 116 | sarbe- | sense_5p_ | sense | L | DNA or | 1501 | attaaaggtttataccttcccaggtaacaaaccaaccaactttccatctcttgaagatgggttctctaaacgaacttta |
| | covirus | SARS2_ | | | RNA | | aaatctgtgtggctgtcactcggctgcatgcttagtgcactcacgcagtataattaataactaattactgtcgttgaca |
| | | UTRuATGv1 | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaacg |
| | | | | | | | aacaaactaaa |
| 117 | sarbe- | sense_5p_ | sense | L | DNA or | 1501 | attaaaggtttataccttcccaggtaacaaaccaaccaactatggatctcttgaagatctgttctctaaacgaacttta |
| | covirus | SARS2_ | | | RNA | | aaatctgtgtggctgtcactcggctgcatgcttagtgcactcacgcagtataattaataactaattactgtcgttgaca |
| | | UTRuATGv2 | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtat |
| | | | | | | | aaattagaaggctatgccttcgaacatatcgtttatggagatttttgacgttcgtgttgttttagatttcatctaaac |
| | | | | | | | gaacaaactaaa |
| 118 | sarbe- | sense_5p_ | sense | L | DNA or | 1501 | attaaaggtttataccttcccaggtaacaaaccaaccaactttcgatctcttgtagatctgtaccatggacgaacttta |
| | covirus | SARS2_ | | | RNA | | aaatctgtgtggctgtcactcggctgcatgcttagtgcactcacgcagtataattaataactaattactgtcgttgaca |
| | | UTRuATGv3 | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaacga |
| | | | | | | | acaaactaaa |
| 119 | sarbe- | sense_5p_ | sense | L | DNA or | 1510 | attaaaggtttataccttcccaggtaacaaaccaaccaactttcgatctcttgtagatctgttctctaaacgaacttta |
| | covirus | SARS2_hp_ATG2 | | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaa |
| | | | | | | | tttacaagaatttaaacccaggagtcaaatggaaattgatttcttagaattagctatggatgaattcattgaacggtat |
| | | | | | | | aaattagaaggctatgccttcgaacatatcgtttatggagatttttgacattttagtttgttcgtttagtttagattt |
| | | | | | | | catctaaacgaacaaactaaa |
| 120 | sarbe- | sense_5p_ | sense | L | DNA or | 1526 | attaaaggtttataccttcccaggtaacaaaccaaccaactttcgatctcttgtagatctgttctctaaacgaacttt |
| | covirus | SARS2_hp_ATG3 | | | RNA | | aaaatctgtgtggctgtcactcggctgcatgcttagtgcactcacgcagtataattaataactaattactgtcgttgac |
| | | | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtat |
| | | | | | | | aaattagaaggctatgccttcgaacatatcgtttatggagatttttgacattttagtttgttcgtttagatgaaatccg |
| | | | | | | | tgttgttttagatttcatctaaacgaacaaactaaa |
| 121 | sarbe- | sense_5p_ | sense | L | DNA or | 1495 | attaaaggtttataccttcccaggtaacaaaccaaccaactttcgatctcttgtagatctgttctctaaacggatttta |
| | covirus | SARS2_ | | | RNA | | aaatctgtgtggctgtcactcggctgcatgcttagtgcactcacgcagtataattaataactaattactgtcgttgaca |
| | | nonactivatable | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaa |
| | | | | | | | tttacaagaatttaaacccaggagtcaaatggaaattgatttcttagaattagctatggatgaattcattgaacggtat |
| | | | | | | | aaattagaaggctatgccttcgaacatatcgtttatggagatttttgacgttcgtgttgttttagatttcatctaaa |
| | | | | | | | aactaaa |
| 122 | sarbe- | sense_5p_ | sense | L | DNA or | 1501 | attaaaggtttataccttcccaggtaacaaaccaaccaactttcgatctcttgtagatctgttctctaaacgaacttt |
| | covirus | SARS2_nsp1_ | | | RNA | | aaaatctgtgtggctgtcactcggctgcatgcttagtgcactcacgcagtataattaataactaattactgtcgttgac |
| | | 124 | | | | | aggacacgagtaactcgtctatcttctgcaggctgcttacggtttcgtccgtgttgcagccgatcatcagcacatcta |
| | | | | | | | ggtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttgctgccaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtat |
| | | | | | | | aaattagaaggctatgccttcgaacatatcgtttatggagatttttgacgttcgtgttgttttagatttcatctaaacg |
| | | | | | | | aacaaactaaa |
| 123 | sarbe- | sense_5p_ | sense | L | DNA or | 1501 | attaaaggtttataccttcccaggtaacaaaccaaccaactttcgatctcttgtagatctgttctctaaacgaacttt |
| | covirus | SARS2_nsp1_ | | | RNA | | aaaatctgtgtggctgtcactcggctgcatgcttagtgcactcacgcagtataattaataactaattactgtcgttgac |
| | | 124_164 | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttgctgccaacggtaataaaggagctggtggccatagttacggcgccgatctaaagtcatttgacttaggcg |
| | | | | | | | acgagcttggcactgatccttatgaagattttcaagaaaactggaacactgccgctagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaaaaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaacga |
| | | | | | | | acaaactaaa |
| 124 | sarbe- | sense_5p_ | sense | L | DNA or | 1501 | attaaaggtttataccttcccaggtaacaaaccaaccaactttcgatctcttgtagatctgttctctaaacgaacttta |
| | covirus | SARS2_nsp1_ | | | RNA | | aaatctgtgtggctgtcactcggctgcatgcttagtgcactcacgcagtataattaataactaattactgtcgttgaca |
| | | 164 | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactgccgctagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaacga |
| | | | | | | | acaaactaaa |
| 125 | sarbe- | sense_5p_ | sense | L | DNA or | 1530 | attaaaggtttataccttcccaggtaacaaaccaaccaactttcgatctcttgtagatctgttctctaaacgaacttta |
| | covirus | SARS2_uATG_ | | | RNA | | aaatctgtgtggctgtcactcggctgcatgcttagtgcactcacgcagtataattaataactaattactgtcgttgaca |
| | | NDegron1 | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgaccaccatgcggggcgacctggccctcctgaag |
| | | | | | | | ctgctcgacctgcggtttcatctaaacgaacaaactaaa |
| 126 | sarbe- | sense_5p_ | sense | L | DNA or | 1533 | attaaaggtttataccttcccaggtaacaaaccaaccaactttcgatctcttgtagatctgttctctaaacgaacttt |
| | covirus | SARS2_uATG_ | | | RNA | | aaaatctgtgtggctgtcactcggctgcatgcttagtgcactcacgcagtataattaataactaattactgtcgttgac |
| | | NDegron2 | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaa |
| | | | | | | | tttacaagaatttaaacccaggagtcaaatggaaattgatttcttagaattagctatggatgaattcattgaacggtat |
| | | | | | | | aaattagaaggctatgccttcgaacatatcgtttatggagatttttgaccaccatgaagatcggcctgggcaggcagaa |
| | | | | | | | gccccctaaagccaccaagtttcatctaaacgaacaaactaaa |
| 127 | sarbe- | sense_5p_ | sense | L | DNA or | 1492 | attaaaggtttataccttcccaggtaacaaaccaaccaactttcgatctcttgtagatctgttctctaaacgaacttta |
| | covirus | SARS2_uATG_ | | | RNA | | aaatctgtgtggctgtcactcggctgcatgcttagtgcactcacgcagtataattaataactaattactgtcgttgaca |
| | | overlap | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaa |
| | | | | | | | tttacaagaatttaaacccaggagtcaaatggaaattgatttcttagaattagctatggatgaattcattgaacggta |
| | | | | | | | taaattagaaggctatgccttcgaacatatcgtttatggagatttttgaccaccatgtttcatctaaacgaacaaact |
| | | | | | | | aaaa |
| 128 | sarbe- | antis_3p_ | sense | R | DNA or | 80 | tttaatttatccggttagagaacagatctacaagagatcgaggttggttggcttttcctgggtaggtaaaaacctaat |
| | covirus | SARS1_short | | | RNA | | at |
| 129 | sarbe- | sense_3p_SARS1 | sense | R | DNA or | 370 | actcatgatgaccacacaaggcagatgggctatgtaaacgttttcgcaattccgtttacgatacatagtctactcttgt |
| | covirus | | | | RNA | | gcagaatgaattctcgtaactaaacagcacaagtaggtttagttaactttaatctcacatagcaatctttaatcaatg |
| | | | | | | | tgtaacattagggaggacttgaaagagccaccacattttcatcgaggccacgcggagtacgatcgagggtacagtga |
| | | | | | | | ataatgctagggagagctgcctatatggaagagccctaatgtgtaaaattaattttagtagtgctatccccatgtgat |
| | | | | | | | tttaatagcttcttaggagaatgacaaaaaaaaaaaaaaaaaaaaaaaaaaaaaaaaa |
| 130 | sarbe- | sense_3p_SARS2 | sense | R | DNA or | 370 | actcatgcagaccacacaaggcagatgggctatataaacgttttcgcttttccgtttacgatatatagtctactcttg |
| | covirus | | | | RNA | | tgcagaatgaattctcgtaactacatagcacaagtagatgtagttaactttaatctcacatagcaatctttaatcagt |
| | | | | | | | gtgtaacattagggaggacttgaaagagccaccacattttcaccgaggccacgcggagtacgatcgagtgtacagtga |
| | | | | | | | acaatgctagggagagctgcctatatggaagagccctaatgtgtaaaattaattttagtagtgctatccccatgtgat |
| | | | | | | | tttaatagcttcttaggagaatgacaaaaaaaaaaaaaaaaaaaaaaaaaaaaaaaaa |
| 131 | sarbe- | sense_3p_SARS2 | sense | R | DNA or | 340 | actcatgcagaccacacaaggcagatgggcatatatagtctactcttgtgcagaatgaattctcgtaactacatagca |
| | covirus | GL_A30_loc10 | | | RNA | | caagtagatgtagttaactttaatctcacatagcaatctttaatcagtgtgtaacattagggaggacttgaaagagcca |
| | | | | | | | ccacattttcaccgaggccacgcggagtacgatcgagtgtacagtgaacaatgctagggagagctgcctatatgg |
| | | | | | | | aagagccctaatgtgtaaaattaattttagtagtgctatccccatgtgattttaatagcttcttaggagaatgacaaa |
| | | | | | | | aaaaaaaaaaaaaaaaaaaaaaaaaaaaaa |
| 132 | sarbe- | sense_3p_SARS2 | sense | R | DNA or | 340 | actcatgcagaccacacaaggcagatgggctatataaacgttttcgcttttccgtttacgatatatagtctactcttg |
| | covirus | GL_A30_loc11 | | | RNA | | tgcagaatgaattctaactttaatctcacatagcaatctttaatcagtgtgtaacattagggaggacttgaaagagcc |
| | | | | | | | accacattttcaccgaggccacgcggagtacgatcgagtgtacagtgaacaatgctagggagagctgcctatatggaa |
| | | | | | | | gagccctaatgtgtaaaattaattttagtagtgctatccccatgtgattttaatagcttcttaggagaatgacaaaaa |
| | | | | | | | aaaaaaaaaaaaaaaaaaaaaaaaaaaa |
| 133 | sarbe- | sense_3p_SARS2 | sense | R | DNA or | 340 | actcatgcagaccacacaaggcagatgggctatataaacgttttcgcttttccgtttacgatatatagtctactcttg |
| | covirus | GL_A30_loc12 | | | RNA | | tgcagaatgaattctcgtaactacatagcacaagtagatgtagttaactttaatctcacatagcaacttgaaagagcc |
| | | | | | | | accacattttcaccgaggccacgcggagtacgatcgagtgtacagtgaacaatgctagggagagctgcctatatgga |
| | | | | | | | agagccctaatgtgtaaaattaattttagtagtgctatccccatgtgattttaatagcttcttaggagaatgacaaaa |
| | | | | | | | aaaaaaaaaaaaaaaaaaaaaaaaaaaaa |
| 134 | sarbe- | sense_3p_SARS2 | sense | R | DNA or | 340 | actcatgcagaccacacaaggcagatgggctatataaacgttttcgcttttccgtttacgatatatagtctactcttg |
| | covirus | GL_A30_loc13 | | | RNA | | tgcagaatgaattctcgtaactacatagcacaagtagatgtagttaactttaatctcacatagcaatctttaatcagt |
| | | | | | | | gtgtaacattagggaggacttgaaggagtacgatcgagtgtacagtgaacaatgctagggagagctgcctatatggaa |
| | | | | | | | gagccctaatgtgtaaaattaattttagtagtgctatccccatgtgattttaatagcttcttaggagaatgacaaaaa |
| | | | | | | | aaaaaaaaaaaaaaaaaaaaaaaaaaaa |
| 135 | sarbe- | sense_3p_SARS2 | sense | R | DNA or | 340 | actcatgcagaccacacaaggcagatgggctatataaacgttttcgcttttccgtttacgatatatagtctactcttg |
| | covirus | GL_Δ30_loc14 | | | RNA | | tgcagaatgaattctcgtaactacatagcacaagtagatgtagttaactttaatctcacatagcaatctttaatcagt |
| | | | | | | | gtgtaacattagggaggacttgaaagagccaccacattttcaccgaggccacgcggagtacgatcggcctatatggaa |
| | | | | | | | gagccctaatgtgtaaaattaattttagtagtgctatccccatgtgattttaatagcttcttaggagaatgacaaaaa |
| | | | | | | | aaaaaaaaaaaaaaaaaaaaaaaaaaaa |
| 136 | sarbe- | antis_3p_ | anti- | L | DNA or | 83 | tttagtttatccggttagagaacagatctacaagagatcgaaagttggttggtttgttacctgggaaggtataaacct |
| | covirus | SARS2_short | sense | | RNA | | ttaat |
| 137 | sarbe- | antis_5p_SARS1 | anti- | L | DNA or | 1607 | ttttttttttgtcattctcctaagaagctattaaaatcacatggggatagcactactaaaattaattttacacattagg |
| | covirus | | sense | | RNA | | gctcttccatataggcagctctccctagcattattcactgtaccctcgatcgtactccgcgtggcctcgatgaaaatg |
| | | | | | | | tggtggctctttcaagtcctccctaatgttacacattgattaaagattgctatgtgagattaaagttaactaaacctac |
| | | | | | | | ttgtgctgtttagttacgagaattcattctgcacaagagtagactatgtatcgtaaacggaattgcgaaaacgtttaca |
| | | | | | | | tagcccatctgccttgtgtggtcatcatgagtttaggcctgagttgagtcagcactgctcatggattgttgcaattgtt |
| | | | | | | | tggagaaatcatccaaatctgcagcaggaagaagagtcacagtttgctgtttcttctgtctctgcggtaaggcttgagt |
| | | | | | | | ttcatcagccttcttctttttgtcctttttaggctctgttggtgggaatgttttgtatgcgtcaatatgcttattcagc |
| | | | | | | | aaaatgacttgatctttgaaatttggatctttgtcatccaatttgatggcacctgtgtaggtcaaccacgttcccgaag |
| | | | | | | | gtgtgacttccatgccaatgcgcgacattccgaagaacgctgaagcgctgggggcaaattgtgcaatttgcggccaat |
| | | | | | | | gtttgtaatcagttccttgtctgattagttcctggtccccaaaatttccttgggtttgttctggaccacgtctgccga |
| | | | | | | | aagcttgtgttacattgtatgctttagtggcagtacgtttttgccgaggcttcttagaagcctcagcagcagatttctt |
| | | | | | | | agtgacagtttggccttgttgttgttggcctttaccagacattttgctctcaagctggttcaatctgtcaagcagcagc |
| | | | | | | | aaagcaagagcagcatcaccgccattgccagccattctagcaggagaagttcccctactgctgcctggagttgaattt |
| | | | | | | | cttgaactgttgcgactacgtgatgaggaacgagaagaggcttgactgccgcctctgctcccttctgcgtagaagcctt |
| | | | | | | | ttggcaatgttgttccttgaggaagttgtagcacgattgcagcattgttagcaggattgcgggtgccaatgtgatcttt |
| | | | | | | | tggtgtattcaaggctccctcagttgcaacccatatgatgccgtctttgttagcaccatagggaagtccagcttctggc |
| | | | | | | | ccagttcctaggtagtagaaataccatcttggactgagatctttcattttaccgtcaccaccacgaattcgtctggtag |
| | | | | | | | ctcttcggtagtagccaatttggtcatctggactgctattggtgttaattggaacgccttgtcctcgagggaatttaa |
| | | | | | | | ggtcttccttgccatgttgagtgagagcggtgaaccaagacgcagtattattgggtaaaccttggggccgacgttgttt |
| | | | | | | | cgttctccattctggttactgccagttgaatctgagggtccaccaaacgtaatgcggggtgcatttcgctgattttggg |
| | | | | | | | tgatcgcgccccactggtccattatcagatta |
| 138 | sarbe- | antis_5p_SARS2 | anti- | L | DNA or | 352 | tttttttttttttttgtcattctcctaagaagctattaaaatcacatggggatagcactactaaaattaattttacaca |
| | covirus | | sense | | RNA | | ttagggctcttccatataggcagctctccctagcattgttcactgtacactcgatcgtactccgcgtggcctcggtgaa |
| | | | | | | | aatgtggtggctctttcaagtcctccctaatgttacacactgattaaagattgctatgtgagattaaagttaactacat |
| | | | | | | | ctacttgtgctatgtagttacgagaattcattctgcacaagagtagactatatatcgtaaacggaaaagcgaaaacgt |
| | | | | | | | ttatatagcccatctgccttgtgtggtctgcatgagt |
| 139 | sarbe- | antis_5p_SARS2 | anti- | L | DNA or | 1607 | ttttttttttgtcattctcctaagaagctattaaaatcacatggggatagcactactaaaattaattttacacattagg |
| | covirus | GL_B | sense | | RNA | | gctcttccatataggcagctctccctagcattgttcactgtacactcgatcgtactccgcgtggcctcggtgaaaatgt |
| | | | | | | | ggtggctctttcaagtcctccctaatgttacacactgattaaagattgctatgtgagattaaagttaactacatctac |
| | | | | | | | ttgtgctatgtagttacgagaattcattctgcacaagagtagactatatatcgtaaacggaaaagcgaaaacgtttat |
| | | | | | | | atagcccatctgccttgtgtggtctgcatgagtttaggcctgagttgagtcagcactgctcatggattgttgcaattg |
| | | | | | | | tttggagaaatcatccaaatctgcagcaggaagaagagtcacagtttgctgtttcttctgtctctgcggtaaggcttga |
| | | | | | | | gtttcatcagccttcttctttttgtcctttttaggctctgttggtgggaatgttttgtatgcgtcaatatgcttattc |
| | | | | | | | agcaaaatgacttgatctttgaaatttggatctttgtcatccaatttgatggcacctgtgtaggtcaaccacgttcccg |
| | | | | | | | aaggtgtgacttccatgccaatgcgcgacattccgaagaacgctgaagcgctgggggcaaattgtgcaatttgcggcca |
| | | | | | | | atgtttgtaatcagttccttgtctgattagttcctggtccccaaaatttccttgggtttgttctggaccacgtctgcc |
| | | | | | | | gaaagcttgtgttacattgtatgctttagtggcagtacgtttttgccgaggcttcttagaagcctcagcagcagattt |
| | | | | | | | cttagtgacagtttggccttgttgttgttggcctttaccagacattttgctctcaagctggttcaatctgtcaagcag |
| | | | | | | | cagcaaagcaagagcagcatcaccgccattgccagccattctagcaggagaagttcccctactgctgcctggagttgaa |
| | | | | | | | tttcttgaactgttgcgactacgtgatgaggaacgagaagaggcttgactgccgcctctgctcccttctgcgtagaagc |
| | | | | | | | cttttggcaatgttgttccttgaggaagttgtagcacgattgcagcattgttagcaggattgcgggtgccaatgtgatc |
| | | | | | | | ttttggtgtattcaaggctccctcagttgcaacccatatgatgccgtctttgttagcaccatagggaagtccagcttc |
| | | | | | | | tggcccagttcctaggtagtagaaataccatcttggactgagatctttcattttaccgtcaccaccacgaattcgtctg |
| | | | | | | | gtagctcttcggtagtagccaatttggtcatctggactgctattggtgttaattggaacgccttgtcctcgagggaat |
| | | | | | | | ttaaggtcttccttgccatgttgagtgagagcggtgaaccaagacgcagtattattgggtaaaccttggggccgacgt |
| | | | | | | | tgttttgatcgcgccccactgcgttctccattctggttactgccagttgaatctgagggtccaccaaacgtaatgcggg |
| | | | | | | | gtgcatttcgctgattttggggtccattatcagatta |
| 140 | sarbe- | antis_5p_SARS2 | anti- | L | DNA or | 1528 | attaaaggtttataccttcccaggtaacaaaccaaccaactttcgatctcttgtagatctgttctctaaccggatttta |
| | covirus | GL_invert_pA | sense | | RNA | | aaatctgtgtggctgtcactcggctgcatgcttagtgcactcacgcagtataattaataactaattactgtcgttgaca |
| | | | | | | | ggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | tgcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaa |
| | | | | | | | tttacaagaatttaaacccaggagtcaaatggaaattgatttcttagaattagctatggatgaattcattgaacggta |
| | | | | | | | taaattagaaggctatgccttcgaacatatcgtttatggagatttttgacgttcgtgttgttttagatttcatctaaa |
| | | | | | | | aactaaattttttttttttttttttttttttttttttttt |
| 141 | sarbe- | antis_5p_SARS2 | anti- | L | DNA or | 1633 | attaaaggtttataccttcccaggtaacaaaccaaccaactttcgatctcttgtagatctgttctctaaccggatttta |
| | covirus | GL_invert_pA_ | sense | | RNA | | aaatctgtgtggctgtcactcggctgcatgcttagtgcactcacgcagtataattaataactaattactgtcgttgaca |
| | | HDVR | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtat |
| | | | | | | | aaattagaaggctatgccttcgaacatatcgtttatggagatttttgacgttcgtgttgttttagatttcatctaaaa |
| | | | | | | | actaaatcgaggcggatcgatccgagctctcccttagccatccgagtggacgacgtcctccttcggatgcccaggtcg |
| | | | | | | | gaccgcgaggaggtggagatgccatgccgacccttttttttttttttttttttttttttttttttt |
| 142 | sarbe- | antis_5p_ | anti- | L | DNA or | 1610 | nnnttttttttttgtcattctcctaagaagctattaaaatcacatggggatagcactactaaaattaattttacacatt |
| | covirus | SARS2_B | sense | | RNA | | agggctcttccatataggcagctctccctagcattgttcactgtacactcgatcgtactccgcgtggcctcggtgaaaa |
| | | | | | | | tgtggtggctctttcaagtcctccctaatgttacacactgattaaagattgctatgtgagattaaagttaactacatc |
| | | | | | | | tacttgtgctatgtagttacgagaattcattctgcacaagagtagactatatatcgtaaacggaaaagcgaaaacgttt |
| | | | | | | | atatagcccatctgccttgtgtggtctgcatgagtttaggcctgagttgagtcagcactgctcatggattgttgcaat |
| | | | | | | | tgtttggagaaatcatccaaatctgcagcaggaagaagagtcacagtttgctgtttcttctgtctctgcggtaaggctt |
| | | | | | | | gagtttcatcagccttcttctttttgtcctttttaggctctgttggtgggaatgttttgtatgcgtcaatatgcttatt |
| | | | | | | | cagcaaaatgacttgatctttgaaatttggatctttgtcatccaatttgatggcacctgtgtaggtcaaccacgttcc |
| | | | | | | | cgaaggtgtgacttccatgccaatgcgcgacattccgaagaacgctgaagcgctgggggcaaattgtgcaatttgcggc |
| | | | | | | | caatgtttgtaatcagttccttgtctgattagttcctggtccccaaaatttccttgggtttgttctggaccacgtctgc |
| | | | | | | | cgaaagcttgtgttacattgtatgctttagtggcagtacgtttttgccgaggcttcttagaagcctcagcagcagatt |
| | | | | | | | tcttagtgacagtttggccttgttgttgttggcctttaccagacattttgctctcaagctggttcaatctgtcaagcag |
| | | | | | | | cagcaaagcaagagcagcatcaccgccattgccagccattctagcaggagaagttcccctactgctgcctggagttgaa |
| | | | | | | | tttcttgaactgttgcgactacgtgatgaggaacgagaagaggcttgactgccgcctctgctcccttctgcgtagaagc |
| | | | | | | | cttttggcaatgttgttccttgaggaagttgtagcacgattgcagcattgttagcaggattgcgggtgccaatgtgatc |
| | | | | | | | ttttggtgtattcaaggctccctcagttgcaacccatatgatgccgtctttgttagcaccatagggaagtccagcttct |
| | | | | | | | ggcccagttcctaggtagtagaaataccatcttggactgagatctttcattttaccgtcaccaccacgaattcgtctgg |
| | | | | | | | tagctcttcggtagtagccaatttggtcatctggactgctattggtgttaattggaacgccttgtcctcgagggaattt |
| | | | | | | | aaggtcttccttgccatgttgagtgagagcggtgaaccaagacgcagtattattgggtaaaccttggggccgacgttg |
| | | | | | | | ttttgatcgcgccccactgcgttctccattctggttactgccagttgaatctgagggtccaccaaacgtaatgcgggg |
| | | | | | | | tgcatttcgctgattttggggtccattatcagatta |
| 143 | sarbe- | antis_5p_ | anti- | L | DNA or | 1528 | attaaaggtttataccttcccaggtaacaaaccaaccaactttcgatctcttgtagatctgttctctaaacgaacttt |
| | covirus | SARS2_invert_ | sense | | RNA | | aaaatctgtgtggctgtcactcggctgcatgcttagtgcactcacgcagtataattaataactaattactgtcgttgac |
| | | pA | | | | | aggacacgagtaactcgtctatcttctgcaggctgcttacggtttcgtccgtgttgcagccgatcatcagcacatctag |
| | | | | | | | gtttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggt |
| | | | | | | | agtgttaaaggtttacaaccatctgtaggtcccaaacaagctagtcttaatggagtcacattaattggagaagccgtaa |
| | | | | | | | aaacacagttcaattattataagaaagttgatggtgttgtccaacaattacctgaaacttactttactcagagtagaa |
| | | | | | | | atttacaagaatttaaacccaggagtcaaatggaaattgatttcttagaattagctatggatgaattcattgaacggta |
| | | | | | | | taaattagaaggctatgccttcgaacatatcgtttatggagatttttgacgttcgtgttgttttagatttcatctaaaa |
| | | | | | | | actaaattttttttttttttttttttttttttttttttt |
| 144 | sarbe- | antis_5p_ | anti- | L | DNA or | 1633 | attaaaggtttataccttcccaggtaacaaaccaaccaactttcgatctcttgtagatctgttctctaaacgaacttta |
| | covirus | SARS2_invert_ | sense | | RNA | | aaatctgtgtggctgtcactcggctgcatgcttagtgcactcacgcagtataattaataactaattactgtcgttgaca |
| | | pA_HDVR | | | | | ggacacgagtaactcgtctatcttctgcaggctgcttacggtttcgtccgtgttgcagccgatcatcagcacatctagg |
| | | | | | | | tttcgtccgggtgtgaccgaaaggtaagatggagagccttgtccctggtttcaacgagaaaacacacgtccaactca |
| | | | | | | | gtttgcctgttttacaggttcgcgacgtgctcgtacgtggctttggagactccgtggaggaggtcttatcagaggcac |
| | | | | | | | gtcaacatcttaaagatggcacttgtggcttagtagaagttgaaaaaggcgttttgcctcaacttgaacagccctatgt |
| | | | | | | | gttcatcaaacgttcggatgctcgaactgcacctcatggtcatgttatggttgagctggtagcagaactcgaaggcat |
| | | | | | | | tcagtacggtcgtagtggtgagacacttggtgtccttgtccctcatgtgggcgaaataccagtggcttaccgcaagg |
| | | | | | | | ttcttcttcgtaagaacggtaataaaggagctggtggccatagttacggcgccgatctaaagtcatttgacttaggcg |
| | | | | | | | acgagcttggcactgatccttatgaagattttcaagaaaactggaacactaaacatagcagtggtgttacccgtgaa |
| | | | | | | | ctcatgcgacagggtgaagtaccagtttctatcattaataacactgtttacacaaaagttgatggtgttgatgtagaat |
| | | | | | | | tgtttgaaaataaaacaacattacctgttaatgtagcatttgagctttgggctaagcgcaacattaaaccagtaccaga |
| | | | | | | | ggtgaaaatactcaataatttgggtgtggacattgctgctaatactgtgatctgggactacaaaagagatgctccagc |
| | | | | | | | acatatatctactattggtgtttgttctatgactgacatagccaagaaaccaactgaaacgatttgtgcaccactcact |
| | | | | | | | gtcttttttgatggtagagttgatggtcaagtagacttatttagaaatgcccgtaatggtgttcttattacagaaggta |
| | | | | | | | gtgttaaaggtttacaaccatctgtaggtcccaaacaagctagtcttaatggagtcacattaattggagaagccgtaaa |
| | | | | | | | aacacagttcaattattataagaaagttgatggtgttgtccaacaattacctgaaacttactttactcagagtagaaat |
| | | | | | | | ttacaagaatttaaacccaggagtcaaatggaaattgatttcttagaattagctatggatgaattcattgaacggtata |
| | | | | | | | aattagaaggctatgccttcgaacatatcgtttatggagatttttgacgttcgtgttgttttagatttcatctaaaaa |
| | | | | | | | ctaaatcgaggcggatcgatccgagctctcccttagccatccgagtggacgacgtcctccttcggatgcccaggtcgg |
| | | | | | | | accgcgaggaggtggagatgccatgccgacccttttttttttttttttttttttttttttttttt |
| 145 | sarbe- | antis_3p_SARS2 | anti- | R | DNA or | 1501 | tttagtttgttcgtttagatgaaatctaaaacaacacgaacgtcaaaaatctccataaacgatatgttcgaaggcatag |
| | covirus | | sense | | RNA | | ccttctaatttataccgttcaatgaattcatccatagctaattctaagaaatcaatttccatttgactcctgggtttaa |
| | | | | | | | attcttgtaaatttctactctgagtaaagtaagtttcaggtaattgttggacaacaccatcaactttcttataataatt |
| | | | | | | | gaactgtgtttttacggcttctccaattaatgtgactccattaagactagcttgtttgggacctacagatggttgtaaa |
| | | | | | | | cctttaacactaccttctgtaataagaacaccattacgggcatttctaaataagtctacttgaccatcaactctaccat |
| | | | | | | | caaaaaagacagtgagtggtgcacaaatcgtttcagttggtttcttggctatgtcagtcatagaacaaacaccaatagt |
| | | | | | | | agatatatgtgctggagcatctcttttgtagtcccagatcacagtattagcagcaatgtccacacccaaattattgagt |
| | | | | | | | attttcacctctggtactggtttaatgttgcgcttagcccaaagctcaaatgctacattaacaggtaatgttgttttat |
| | | | | | | | tttcaaacaattctacatcaacaccatcaacttttgtgtaaacagtgttattaatgatagaaactggtacttcaccctg |
| | | | | | | | tcgcatgagttcacgggtaacaccactgctatgtttagtgttccagttttcttgaaaatcttcataaggatcagtgcca |
| | | | | | | | agctcgtcgcctaagtcaaatgactttagatcggcgccgtaactatggccaccagctcctttattaccgttcttacgaa |
| | | | | | | | gaagaaccttgcggtaagccactggtatttcgcccacatgagggacaaggacaccaagtgtctcaccactacgaccgta |
| | | | | | | | ctgaatgccttcgagttctgctaccagctcaaccataacatgaccatgaggtgcagttcgagcatccgaacgtttgat |
| | | | | | | | gaacacatagggctgttcaagttgaggcaaaacgcctttttcaacttctactaagccacaagtgccatctttaagatgt |
| | | | | | | | tgacgtgcctctgataagacctcctccacggagtctccaaagccacgtacgagcacgtcgcgaacctgtaaaacaggc |
| | | | | | | | aaactgagttggacgtgtgttttctcgttgaaaccagggacaaggctctccatcttacctttcggtcacacccggacga |
| | | | | | | | aacctagatgtgctgatgatcggctgcaacacggacgaaaccgtaagcagcctgcagaagatagacgagttactcgtg |
| | | | | | | | tcctgtcaacgacagtaattagttattaattatactgcgtgagtgcactaagcatgcagccgagtgacagccacacaga |
| | | | | | | | ttttaaaatccggttagagaacagatctacaagagatcgaaagttggttggtttgttacctgggaaggtataaacctt |
| | | | | | | | taat |
| 146 | sarbe- | antis_3p_SARS2 | anti- | R | DNA or | 1501 | tttagtttatccggttagatgaaatctaaaacaacacgaacgtcaaaaatctccataaacgatatgttcgaaggcatag |
| | covirus | GL | sense | | RNA | | ccttctaatttataccgttcaatgaattcatccatagctaattctaagaaatcaatttccatttgactcctgggtttaa |
| | | | | | | | attcttgtaaatttctactctgagtaaagtaagtttcaggtaattgttggacaacaccatcaactttcttataataat |
| | | | | | | | tgaactgtgtttttacggcttctccaattaatgtgactccattaagactagcttgtttgggacctacagatggttgtaa |
| | | | | | | | acctttaacactaccttctgtaataagaacaccattacgggcatttctaaataagtctacttgaccatcaactctacc |
| | | | | | | | atcaaaaaagacagtgagtggtgcacaaatcgtttcagttggtttcttggctatgtcagtcatagaacaaacaccaata |
| | | | | | | | gtagatatatgtgctggagcatctcttttgtagtcccagatcacagtattagcagcaatgtccacacccaaattattga |
| | | | | | | | gtattttcacctctggtactggtttaatgttgcgcttagcccaaagctcaaatgctacattaacaggtaatgttgtttt |
| | | | | | | | attttcaaacaattctacatcaacaccatcaacttttgtgtaaacagtgttattaatgatagaaactggtacttcaccc |
| | | | | | | | tgtcgcatgagttcacgggtaacaccactgctatgtttagtgttccagttttcttgaaaatcttcataaggatcagtgc |
| | | | | | | | caagctcgtcgcctaagtcaaatgactttagatcggcgccgtaactatggccaccagctcctttattaccgttcttacg |
| | | | | | | | aagaagaaccttgcggtaagccactggtatttcgcccacatgagggacaaggacaccaagtgtctcaccactacgaccg |
| | | | | | | | tactgaatgccttcgagttctgctaccagctcaaccataacatgaccatgaggtgcagttcgagcatccgaacgtttga |
| | | | | | | | tgaacacatagggctgttcaagttgaggcaaaacgcctttttcaacttctactaagccacaagtgccatctttaagatg |
| | | | | | | | ttgacgtgcctctgataagacctcctccacggagtctccaaagccacgtacgagcacgtcgcgaacctgtaaaacaggc |
| | | | | | | | aaactgagttggacgtgtgttttctcgttgaaaccagggacaaggctctccatcttacctttcggtcacacccggacga |
| | | | | | | | aacctagatgtgctgatgatcggctgcaacacggacgaaaccgtaagcagcctgcagaagatagacgagttactcgtg |
| | | | | | | | tcctgtcaacgacagtaattagttattaattatactgcgtgagtgcactaagcatgcagccgagtgacagccacacag |
| | | | | | | | attttaaaatccggttagagaacagatctacaagagatcgaaagttggttggtttgttacctgggaaggtataaacct |
| | | | | | | | ttaat |
| 147 | sarbe- | antis_3p_ | anti- | R | DNA or | 957 | ggttgtggccatattatcatcgtgtttttcaaaggaaaaccacgtccccgtggttcggggggcctagacgtttttttaa |
| | covirus | SARS2_ | sense | | RNA | | cctcgactaaacacatgtaaagcatgtgcaccgaggccccagatcagatcccatacaatggggtaccttctgggc |
| | | invert_IRES | | | | | atccttcagccccttgttgaatacgcttgaggagagccatttgactctttccacaactatccaactcacaacgtggcac |
| | | | | | | | tggggttgtgccgcctttgcaggtgtatcttatacacgtggcttttggccgcagaggcacctgtcgccaggtggggg |
| | | | | | | | gttccgctgcctgcaaagggtcgctacagacgttgtttgtcttcaagaagcttccagaggaactgcttccttcacgac |
| | | | | | | | attcaacagaccttgcattcctttggcgagaggggaaagacccctaggaatgctcgtcaagaagacagggccag |
| | | | | | | | gtttccgggccctcacattgccaaaagacggcaatatggtggaaaataacatatagacaaacgcacaccggcctta |
| | | | | | | | ttccaagcggcttcggccagtaacgttagggggggggggggagaggggactcatgcagaccacacaaggca |
| | | | | | | | gatgggctatataaacgttttcgcttttccgtttacgatatatagtctactcttgtgcagaatgaattctcgtaactac |
| | | | | | | | gatacacaagtagatgtagttaactttaatctcacatagcaatctttaatcagtgtgtaacattagggaggacttgaaa |
| | | | | | | | gagccaccacattttcaccgaggccacgcggagtacgatcgagtgtacagtgaacaatgctagggagagctgcctata |
| | | | | | | | tggaagagccctaatgtgtaaaattaattttagtagtgctatccccatgtgattttaatagcttcttaggagaatgac |
| | | | | | | | aaaaaaaaaaaaaaaaaaaaaaaaaaaaaaaaa |
| 148 | RSV | sense_5p_RSV | sense | L | DNA or | 98 | acgcgaaaaaatgcgtacaacaaacttgcataaaccaaaaaaatggggcaaataagaatttgataagtaccactta |
| | | | | | RNA | | aatttaactcccttgcttagcg |
| 149 | RSV | sense_5p_RSV_ | sense | L | DNA or | 53 | acgcgaaaaaatgcgtacaacaaacttgcataaaccaaaaaaatggggcaaat |
| | | short | | | RNA | | |
| 150 | RSV | sense_5p_RSV_ | sense | L | DNA or | 55 | acgagaaaaaaagtgtcaaaaactaatatctcgtaaccaaaaaaatggggcaaat |
| | | short_3610 | | | RNA | | |
| 151 | RSV | sense_5p_RSV_ | sense | L | DNA or | 56 | acgcgaaaaaatgcgtacaacaaacttgcataaaccaaaaaaatggggcaaatctt |
| | | short_add_agg | | | RNA | | |
| 152 | RSV | sense_5p_RSV_ | sense | L | DNA or | 58 | acgagaaaaaaagtgtcaaaaactaatatctcgtaaccaaaaaaatggggcaaatctt |
| | | short_agg_3610 | | | RNA | | |
| 153 | RSV | sense_5p_RSV_ | sense | L | DNA or | 54 | ggggcaaataagaatttgataagtaccacttaaatttaactcccttgcttagcg |
| | | Δleader | | | RNA | | |
| 154 | RSV | sense_3p_RSV | sense | R | DNA or | 227 | tgaataaagatcttataataaaaattcccatagctatacactaacactgtattcaattatagttattaaaaattaaaa |
| | | | | | RNA | | atcgtacgattttttaaataacttttagtgaactaatcctaaagttatcattttaatcttggaggaataaatttaaac |
| | | | | | | | cctaatctaattggtttatatgtgtattaactaaattacgagatattagtttttgacactttttttctcgt |
| 155 | RSV | sense 3p_RSV_ | sense | R | DNA or | 52 | agttattaaaaataaattacgagatattagtttttgacactttttttctcgt |
| | | short | | | RNA | | |
| 156 | RSV | sense_3p_RSV_ | sense | R | DNA or | 112 | tgaataaagatcttataataaaaattcccatagctatacactaacactgtattcaattatagttattaaaaattaaaa |
| | | Δ115 | | | RNA | | atcgtacgattttttaaataacttttagtgaact |
| 157 | RSV | sense_3p_RSV_ | sense | R | DNA or | 171 | tgaataaagatcttataataaaaattcccatagctatacactaacactgtattcaattatagttattaaaaattaaaa |
| | | Δ56 | | | RNA | | atcgtacgattttttaaataacttttagtgaactaatcctaaagttatcattttaatcttggaggaataaatttaaac |
| | | | | | | | cctaatctaattggt |
| 158 | RSV | antis_5p_RSV | anti- | L | DNA or | 227 | acgagaaaaaaagtgtcaaaaactaatatctcgtaatttagttaatacacatataaaccaattagattagggtttaaa |
| | | | sense | | RNA | | tttattcctccaagattaaaatgataactttaggattagttcactaaaagttatttaaaaaatcgtacgatttttaat |
| | | | | | | | ttttaataactataattgaatacagtgttagtgtatagctatgggaatttttattataagatctttattca |
| 159 | RSV | antis_5p_RSV_ | anti- | L | DNA or | 248 | agctgacacgctgtcctctggacgagaaaaaaagtgtcaaaaactaatatctcgtaatttagttaatacacatataaa |
| | | add_021 nt | sense | | RNA | | ccaattagattagggtttaaatttattcctccaagattaaaatgataactttaggattagttcactaaaagttattta |
| | | | | | | | aaaaatcgtacgatttttaatttttaataactataattgaatacagtgttagtgtatagctatgggaatttttattat |
| | | | | | | | aagatctttattca |
| 160 | RSV | antis_5p_RSV_ | anti- | L | DNA or | 278 | agctgacacgctgtcctctggcgacctgtcgtcggagaggttgggcctccgacgagaaaaaaagtgtcaaaaact |
| | | add_051 nt | sense | | RNA | | aatatctcgtaatttagttaatacacatataaaccaattagattagggtttaaatttattcctccaagattaaaatgat |
| | | | | | | | aactttaggattagttcactaaaagttatttaaaaaatcgtacgatttttaatttttaataactataattgaatacagt |
| | | | | | | | gttagtgtatagctatgggaatttttattataagatctttattca |
| 161 | RSV | antis_5p_RSV_ | anti- | L | DNA or | 328 | agctgacacgctgtcctctggcgacctgtcgtcggagaggttgggcctccggatgcgcgcggggctctggcctc |
| | | add_101 nt | sense | | RNA | | acggtgaccggctagccggccgcgctcacgagaaaaaaagtgtcaaaaactaatatctcgtaatttagttaataca |
| | | | | | | | catataaaccaattagattagggtttaaatttattcctccaagattaaaatgataactttaggattagttcactaaaa |
| | | | | | | | gttatttaaaaaatcgtacgatttttaatttttaataactataattgaatacagtgttagtgtatagctatgggaatt |
| | | | | | | | tttattataagatctttattca |
| 162 | RSV | antis_5p_RSV_ | anti- | L | DNA or | 461 | gtgtcctctggcgacctgtcggctgtcctctggcgacctgtcgtcggagaggttgggccgtcgttcggagaggttg |
| | | add_234nt | sense | | RNA | | ggcctccggatgcgcgcagctgacacgctgtcctctggcgacctgtgggctctggcctcacggtgaccggctag |
| | | | | | | | ccggccgcgctccgtcggagaggttgggcctccggatgcgcgcggggctctggcctcacggtgaccggctagc |
| | | | | | | | cggccgcgctcacgagaaaaaaagtgtcaaaaactaatatctcgtaatttagttaatacacatataaaccaattagat |
| | | | | | | | tagggtttaaatttattcctccaagattaaaatgataactttaggattagttcactaaaagttatttaaaaaatcgta |
| | | | | | | | cgatttttaatttttaataactataattgaatacagtgttagtgtatagctatgggaatttttattataagatcttta |
| | | | | | | | ttca |
| 163 | RSV | antis_5p_RSV_ | anti- | L | DNA or | 230 | aggacgagaaaaaaagtgtcaaaaactaatatctcgtaatttagttaatacacatataaaccaattagattagggttta |
| | | add_agg | sense | | RNA | | aatttattcctccaagattaaaatgataactttaggattagttcactaaaagttatttaaaaaatcgtacgattttta |
| | | | | | | | atttttaataactataattgaatacagtgttagtgtatagctatgggaatttttattataagatctttattca |
| 164 | RSV | antis_5p_RSV_ | anti- | L | DNA or | 227 | aggagaaaaaaagtgtcaaaaactaatatctcgtaatttagttaatacacatataaaccaattagattagggtttaaa |
| | | ag | sense | | RNA | | tttattcctccaagattaaaatgataactttaggattagttcactaaaagttatttaaaaaatcgtacgatttttaat |
| | | | | | | | ttttaataactataattgaatacagtgttagtgtatagctatgggaatttttattataagatctttattca |
| 165 | RSV | antis_5p_RSV_ | anti- | L | DNA or | 167 | acgagaaaaaaagtgtcaaaaactaatatctcgtaatttagttaatacacatataaaccaattagattagggtttaaa |
| | | no_flank | sense | | RNA | | tttattcctccaagattaaaatgataactttaggattagttcactaaaagttatttaaaaaatcgtacgatttttaatt |
| | | | | | | | tttaataact |
| 166 | RSV | antis_5p_RSV_ | anti- | L | DNA or | 52 | acgagaaaaaaagtgtcaaaaactaatatctcgtaatttatttttaataact |
| | | short | sense | | RNA | | |
| 419 | RSV | antis_5p_RSV_ | anti- | L | DNA or | 55 | aggacgagaaaaaaagtgtcaaaaactaatatctcgtaatttatttttaataact |
| | | short_add_agg | sense | | RNA | | |
| 167 | RSV | antis_5p_RSV_ | anti- | L | DNA or | 112 | agttcactaaaagttatttaaaaaatcgtacgatttttaatttttaataactataattgaatacagtgttagtgtata |
| | | Δ115 | sense | | RNA | | gctatgggaatttttattataagatctttattca |
| 168 | RSV | antis_5p_RSV_ | anti- | L | DNA or | 171 | accaattagattagggtttaaatttattcctccaagattaaaatgataactttaggattagttcactaaaagttattt |
| | | Δ56 | sense | | RNA | | aaaaaatcgtacgatttttaatttttaataactataattgaatacagtgttagtgtatagctatgggaatttttatta |
| | | | | | | | taagatctttattca |
| 169 | RSV | antis_3p_RSV | anti- | R | DNA or | 98 | cgctaagcaagggagttaaatttaagtggtacttatcaaattcttatttgccccatttttttggtttatgcaagtttg |
| | | | sense | | RNA | | ttgtacgcattttttcgcgt |
| 170 | RSV | antis_3p_RSV_ | anti- | R | DNA or | 100 | cgctaagcaagggagttaaatttaagtggtacttatcaaattcttatttgccccatttttttggttacgagatattag |
| | | 36 TrC | sense | | RNA | | tttttgacactttttttctcgt |
| 171 | RSV | antis_3p_RSV_ | anti- | R | DNA or | 121 | cgctaagcaagggagttaaatttaagtggtacttatcaaattcttatttgccccatttttttggttacgagatattag |
| | | 36 TrC_ | sense | | RNA | | tttttgacactttttttctcgtagctgacacgctgtcctctgg |
| | | add_021 nt | | | | | |
| 172 | RSV | antis_3p_RSV_ | anti- | R | DNA or | 151 | cgctaagcaagggagttaaatttaagtggtacttatcaaattcttatttgccccatttttttggttacgagatattag |
| | | 36 TrC_ | sense | | RNA | | tttttgacactttttttctcgtagctgacacgctgtcctctggcgacctgtcgtcggagaggttgggcctccg |
| | | add_051 nt | | | | | |
| 173 | RSV | antis_3p_RSV_ | anti- | R | DNA or | 201 | cgctaagcaagggagttaaatttaagtggtacttatcaaattcttatttgccccatttttttggttacgagatatta |
| | | 36 TrC_ | sense | | RNA | | gtttttgacactttttttctcgtagctgacacgctgtcctctggcgacctgtcgtcggagaggttgggcctccggatg |
| | | add_101 nt | | | | | cgcgcggggctctggcctcacggtgaccggctagccggccgcgctc |
| 174 | RSV | antis_3p_RSV_ | anti- | R | DNA or | 374 | cgctaagcaagggagttaaatttaagtggtacttatcaaattcttatttgccccatttttttggttacgagatattag |
| | | 36 TrC_ | sense | | RNA | | tttttgacactttttttctcgtctccggatgcgcgcagctgacacgctgtcctctggcggagacgacctgtcgttcgg |
| | | add_274 nt | | | | | agaggttgggcctccggatgcgcgcagctgacacgctgtcctctggcgacctgtgggctctggcctcacggtgaccggc |
| | | | | | | | tagccggccgcgctccgtcggagaggttgggcctccggatgcgcgcggggctctggcctcacggtgaccggct |
| | | | | | | | agccggccgcgctccggatgcgcgcggggctctggcctcacggtgaccggctagccggccgcgctc |
| 175 | RSV | antis_3p_RSV_ | anti- | R | DNA or | 100 | cgctaagcaagggagttaaatttaagtggtacttatcaaattcttatttgccccatttttttggttacgagatattag |
| | | 36 TrC_ag | sense | | RNA | | tttttgacactttttttctcct |
| 176 | RSV | antis_3p_RSV_ | anti- | R | DNA or | 56 | tatttgccccatttttttggttacgagatattagtttttgacactttttttctcgt |
| | | 36 TrC_ | sense | | RNA | | |
| | | no_flank | | | | | |
| 177 | RSV | antis_3p_RSV_ | anti- | R | DNA or | 53 | atttgccccatttttttggtttatgcaagtttgttgtacgcattttttcgcgt |
| | | short | sense | | RNA | | |
| 178 | RSV | antis_3p_RSV_ | anti- | R | DNA or | 54 | cgctaagcaagggagttaaatttaagtggtacttatcaaattcttatttgcccc |
| | | Δleader | sense | | RNA | | |
| 420 | RSV | antis_3p_RSV_ | anti- | R | DNA or | 55 | atttgccccatttttttggttacgagatattagtttttgacactttttttctcgt |
| | | short_36TrC | sense | | RNA | | |
| 179 | HPIV1 | sense_3p_ | sense | R | DNA or | 119 | accaaacaagaggaaaaacttgtttggaatatataataatattaaatagtattttagggttaaagtaatactttaaag |
| | | HPIV1_FR2011 | | | RNA | | ggacaagtcacagacatttgatcttagtataaatttttata |
| 180 | HPIV1 | sense_5p_ | sense | L | DNA or | 157 | tcttatatcttctcatctttattatctaatttgtttaaagagatgagttaacaagataagaaatccctttaactgact |
| | | HPIV1_FR2011 | | | RNA | | cataaaaacatagtaagaaaaacttacaacagacaagagtattaataatatatcgatatttcttaaactcttgtctggt |
| 181 | HPIV1 | antis_5p_ | anti- | L | DNA or | 157 | accagacaagagtttaagaaatatcgatatattattaatactcttgtctgttgtaagtttttcttactatgtttttat |
| | | HPIV1_FR2011 | sense | | RNA | | gagtcagttaaagggatttcttatcttgttaactcatctctttaaacaaattagataataaagatgagaagatataag |
| | | | | | | | a |
| 182 | HPIV1 | antis_5p_ | anti- | L | DNA or | 163 | aggaccagacaagagtttaagaaatatcgatatattattaatactcttgtctgttgtaagtttttcttactatgtttt |
| | | HPIV1_FR2011_ | sense | | RNA | | tatgagtcagttaaagggatttcttatcttgttaactcatctctttaaacaaattagataataaagatgagaagatat |
| | | add_agg | | | | | aagatcc |
| 183 | HPIV1 | antis_5p_ | anti- | L | DNA or | 157 | accagacaagagtttaagaaatatcgagatattattaatactcttgtctgttgtaagtttttcttactatgtttttat |
| | | HPIV1_FR2011_ | sense | | RNA | | gagtcagttaaagggatttcttatcttgttaactcatctctttaaacaaattagataataaagatgagaagatataag |
| | | t28g | | | | | a |
| 184 | HPIV1 | antis_3p_ | anti- | R | DNA or | 119 | tataaaaatttatactaagatcaaatgtctgtgacttgtccctttaaagtattactttaaccctaaaatactatttaa |
| | | HPIV1_FR2011 | sense | | RNA | | tattattatatattccaaacaagtttttcctcttgtttggt |
| 185 | HPIV3 | sense_3p_ | sense | R | DNA or | 110 | accaaacaagagaagagacttgtttggaaatataaatttaaattaaaattaacttaggattaaagacattgactagaa |
| | | HPIV3_Wash | | | RNA | | ggtcaagaaaagggaactctataatttcaaaa |
| 186 | HPIV3 | sense_5p_ | sense | L | DNA or | 112 | ataaatacaataaagatatatcttaacctttatctttgagcccaaggatagacaaaaagtaagaaaaacatgtaatat |
| | | HPIV3_Wash | | | RNA | | atatataccaagcagagttcttctcttgtttggt |
| 187 | HPIV3 | antis_5p_ | anti- | L | DNA or | 112 | accaaacaagagaagaactctgcttggtatatatatattacatgtttttcttactttttgtctatccttgggctcaaa |
| | | HPIV3_Wash | sense | | RNA | | gataaaggttaagatatatctttattgtatttat |
| 188 | HPIV3 | antis_5p_ | anti- | L | DNA or | 118 | aggaccaaacaagagaagaactctgcttggtatatatatattacatgtttttcttactttttgtctatccttgggctc |
| | | HPIV3_Wash_ | sense | | RNA | | aaagataaaggttaagatatatctttattgtatttatgtc |
| | | add_agg | | | | | |
| 189 | HPIV3 | antis_5p_ | anti- | L | DNA or | 112 | accaaacaagagaagaactctgcttgggatatatatattacatgtttttcttactttttgtctatccttgggctcaaa |
| | | HPIV3_Wash_ | sense | | RNA | | gataaaggttaagatatatctttattgtatttat |
| | | t28g | | | | | |
| 190 | HPIV3 | antis_3p_ | anti- | R | DNA or | 110 | ttttgaaattatagagttcccttttcttgaccttctagtcaatgtctttaatcctaagttaattttaatttaaattta |
| | | HPIV3_Wash | sense | | RNA | | tatttccaaacaagtctcttctcttgtttggt |
| 191 | HMPV | sense_3p_AMPV | sense | R | DNA or | 53 | acgagaaaaaaacgcatataagacaacttccaaacaaaacgggacaagtgaaa |
| | | | | | RNA | | |
| 192 | HMPV | sense_3p_ | sense | R | DNA or | 53 | acgcgaaaaaaacgcgtataaattaaattccaaacaaaacgggacaagtgaaa |
| | | HMPV_NL | | | RNA | | |
| 193 | HMPV | sense_5p_AMPV | sense | L | DNA or | 241 | cagtgctgaacttaaaaagctaatcaaagtgacaggttacatgctgatgagtaagaaatgatggaaggaaattacct |
| | | | | | RNA | | taagaaattacttatagtgaaggcttaaaacttacatatccatgctgaagaatactaaagtgatagttacttaaactt |
| | | | | | | | aataatctgaatatgaagcacacaactagcaattagttatttaaaaatgaggaactaaaattggatgaatacggtttt |
| | | | | | | | tttgccgt |
| 194 | HMPV | sense_5p_ | sense | L | DNA or | 241 | aaaatgatgaagatgacaaaatagatgacaacttcatactattctaaattaattatttgattatgcaattatatgata |
| | | HMPV_NL | | | RNA | | gttaattaaaataataatgataatgattaaccataatctcacacaactgagaaaataatcgtctaacagtttagttga |
| | | | | | | | tcattagttatttaaaattataaaatagtaactaactgataaaaaatcagaaattgaaattgaatgtatacggtttt |
| | | | | | | | tttgccgt |
| 195 | HMPV | antis_5p_AMPV | anti- | L | DNA or | 241 | acggcaaaaaaaccgtattcatccaattttagttcctcatttttaaataactaattgctagttgtgtgcttcatattc |
| | | | sense | | RNA | | agattattaagtttaagtaactatcactttagtattcttcagcatggatatgtaagttttaagccttcactataagta |
| | | | | | | | atttcttaaggtaatttccttccatcatttcttactcatcagcatgtaacctgtcactttgattagctttttaagttc |
| | | | | | | | agcactg |
| 196 | HMPV | antis_5p_HMPV | anti- | L | DNA or | 241 | acggcaaaaaaaccgtatacattcaatttcaatttctgattttttatcagttagttactattttataattttaaataa |
| | | NL | sense | | RNA | | ctaatgatcaactaaactgttagacgattattttctcagttgtgtgagattatggttaatcattatcattattatttt |
| | | | | | | | aattaactatcatataattgcataatcaaataattaatttagaatagtatgaagttgtcatctattttgtcatcttca |
| | | | | | | | tcatttt |
| 197 | HMPV | antis_5p_ | anti- | L | DNA or | 247 | aggacggcaaaaaaaccgtatacattcaatttcaatttctgattttttatcagttagttactattttataattttaaat |
| | | HMPV_NL_add_ | sense | | RNA | | aactaatgatcaactaaactgttagacgattattttctcagttgtgtgagattatggttaatcattatcattattattt |
| | | agg | | | | | taattaactatcatataattgcataatcaaataattaatttagaatagtatgaagttgtcatctattttgtcatcttc |
| | | | | | | | atcatttttcc |
| 198 | HMPV | antis_5p_ | anti- | L | DNA or | 151 | acggcaaaaaaaccgtatacattcaatttcaatttctgattttttatcagttagttactattttataattttaaataac |
| | | HMPV_NL_s2 | sense | | RNA | | taatgatcaactaaactgttagacgattattttctcagttgtgtgagattatggttaatcattatcattatt |
| 199 | HMPV | antis_5p_ | anti- | L | DNA or | 157 | aggacggcaaaaaaaccgtatacattcaatttcaatttctgattttttatcagttagttactattttataattttaaa |
| | | HMPV_NL_s2_ | sense | | RNA | | taactaatgatcaactaaactgttagacgattattttctcagttgtgtgagattatggttaatcattatcattattac |
| | | add_agg | | | | | c |
| 200 | HMPV | antis_3p_AMPV | anti- | R | DNA or | 53 | tttcacttgtcccgttttgtttggaagttgtcttatatgcgtttttttctcgt |
| | | | sense | | RNA | | |
| 201 | HMPV | antis_3p_ | anti- | R | DNA or | 53 | tttcacttgtcccgttttgtttggaatttaatttatacgcgtttttttcgcgt |
| | | HMPV_NL | sense | | RNA | | |
| 202 | NIV | sense_3p_NiV_ | sense | R | DNA or | 112 | accaaacaagggagaatatggatacgttaaaatatataacgtatttttaaaacttaggaaccaagacaaacacttttg |
| | | UMMC1 | | | RNA | | gtcttggtattggatcctcaagaaatatatcatc |
| 203 | NIV | sense_5p_NiV_ | sense | L | DNA or | 98 | taaccttccaaatccaagaccaactgataacttatgttgatctaaggttcagttattaagaaaaacttaataacgatt |
| | | UMMC1 | | | RNA | | cttctttacccttgttcggt |
| 204 | NiV | antis_5p_NiV_ | anti- | L | DNA or | 98 | accgaacaagggtaaagaagaatcgttattaagtttttcttaataactgaaccttagatcaacataagttatcagttg |
| | | UMMC1 | sense | | RNA | | gtcttggatttggaaggtta |
| 205 | NIV | antis_5p_NiV_ | anti- | L | DNA or | 104 | aggaccgaacaagggtaaagaagaatcgttattaagtttttcttaataactgaaccttagatcaacataagttatcag |
| | | UMMC1_add_agg | sense | | RNA | | ttggtcttggatttggaaggttaaac |
| 206 | NIV | antis_3p_NiV_ | anti- | R | DNA or | 112 | gatgatatatttcttgaggatccaataccaagaccaaaagtgtttgtcttggttcctaagttttaaaaatacgttata |
| | | UMMC1 | sense | | RNA | | tattttaacgtatccatattctcccttgtttggt |
| 207 | HeV | sense_5p_HeV_ | sense | L | DNA or | 98 | cgaactaccacacccatcaccaatgaagcccgaacaagtctacatagatcagttattaagaaaaacttaataacgat |
| | | AUS | | | RNA | | ctctctttacccttgttcggt |
| 208 | HeV | sense_3p_HeV_ | sense | R | DNA or | 112 | accgaacaaggggaaatatggatacgtgttaaaaaactgcgtatgtttaaaacttaggaaccaagacagtgacaatt |
| | | AUS | | | RNA | | ggtcttggtattggacaattgttcaaggttccaaa |
| 209 | HeV | antis_5p_HeV_ | anti- | L | DNA or | 98 | accgaacaagggtaaagagagatcgttattaagtttttcttaataactgatctatgtagacttgttcgggcttcattg |
| | | AUS | sense | | RNA | | gtgatgggtgtggtagttcg |
| 210 | HeV | antis_5p_HeV_ | anti- | L | DNA or | 104 | aggaccgaacaagggtaaagagagatcgttattaagtttttcttaataactgatctatgtagacttgttcgggcttca |
| | | AUS_add_agg | sense | | RNA | | ttggtgatgggtgtggtagttcgatt |
| 211 | HeV | antis_3p_HeV_ | anti- | R | DNA or | 112 | tttggaaccttgaacaattgtccaataccaagaccaattgtcactgtcttggttcctaagttttaaacatacgcagtt |
| | | AUS | sense | | RNA | | ttttaacacgtatccatatttccccttgttcggt |
| 212 | HBV | sense_5p_HBV_ | sense | L | DNA or | 1626 | agcaccatgcaactttttcacctctgcctaatcatctcttgttcatgtcctactgttcaagcctccaagctgtgcctt |
| | | 21 | | | RNA | | gggtggctttggggcatggacatcgacccttataaagaatttggagctactgtggagttactctcgtttttgccttctg |
| | | | | | | | acttctttccttcagtacgagatcttctagataccgcctcagctctgtatcgggaagccttagagtctcctgagcatt |
| | | | | | | | gttcacctcaccatactgcactcaggcaagcaattctttgctggggggaactaatgactctagctacctgggtgggtgt |
| | | | | | | | taatttggaagatccagcatctagagacctagtagtcagttatgtcaacactaatatgggcctaaagttcaggcaact |
| | | | | | | | cttgtggtttcacatttcttgtctcacttttggaagagaaaccgttatagagtatttggtgtctttcggagtgtggatt |
| | | | | | | | cgcactcctccagcttatagaccaccaaatgcccctatcctatcaacacttccggaaactactgttgttagacgacgag |
| | | | | | | | gcaggtcccctagaagaagaactccctcgcctcgcagacgaaggtctcaatcgccgcgtcgcagaagatctcaatctcg |
| | | | | | | | ggaacctcaatgttagtattccttggactcataaggtggggaactttactggtctttattcttctactgtacctgtct |
| | | | | | | | ttaatcctcattggaaaacaccatcttttcctaatatacatttacaccaagacattatcaaaaaatgtgaacagtttgt |
| | | | | | | | aggcccacttacagttaatgagaaaagaagattgcaattgattatgcctgctaggttttatccaaaggttaccaaatat |
| | | | | | | | ttaccattggataagggtattaaaccttattatccagaacatctagttaatcattacttccaaactagacactatttac |
| | | | | | | | acactctatggaaggcgggtatattatataagagagaaacaacacatagcgcctcattttgtgggtcaccatattctt |
| | | | | | | | gggaacaagatctacagcatggggcagaatctttccaccagcaatcctctgggattctttcccgaccaccagttggat |
| | | | | | | | ccagccttcagagcaaacacagcaaatccagattgggacttcaatcccaacaaggacactggccagacgccaacaaggt |
| | | | | | | | aggagctggagcattcgggctgggtttcaccccaccgcacggaggccttttggggtggagccctcaggctcagggcata |
| | | | | | | | ctacaaactttgccagcaaatccgcctcctgcctccaccaatcgccagacaggaaggcagcctaccccgctgtctcc |
| | | | | | | | acctttgagaaacactcatcctcaggccatgcagtggaattccacaacctttcaccaaactctgcaagatcccagag |
| | | | | | | | tgagaggcctgtatttccctgctggtggctccagttcaggagcagtaaaccctgttccgactactgcctctcccttatc |
| | | | | | | | gtcaatcttctcgaggattggggaccctgcgctgaacattcctaacaacttatgagttcactttttttgcaggttgagg |
| | | | | | | | gcagaggaagtctgctaacatgcggtgacgtcgaggagaatcctggcccaactagt |
| 213 | HBV | sense_5p_HBV_ | sense | L | DNA or | 1591 | agcaccatgcaactttttcacctctgcctaatcatctcttgttcatgtcctactgttcaagcctccaagctgtgcctt |
| | | 31 | | | RNA | | gggtggctttggggcatggacatcgacccttataaagaatttggagctactgtggagttactctcgtttttgccttct |
| | | | | | | | gacttctttccttcagtacgagatcttctagataccgcctcagctctgtatcgggaagccttagagtctcctgagcat |
| | | | | | | | tgttcacctcaccatactgcactcaggcaagcaattctttgctggggggaactaatgactctagctacctgggtgggtg |
| | | | | | | | ttaatttggaagatccagcatctagagacctagtagtcagttatgtcaacactaatatgggcctaaagttcaggcaact |
| | | | | | | | cttgtggtttcacatttcttgtctcacttttggaagagaaaccgttatagagtatttggtgtctttcggagtgtggatt |
| | | | | | | | cgcactcctccagcttatagaccaccaaatgcccctatcctatcaacacttccggaaactactgttgttagacgacgag |
| | | | | | | | gcaggtcccctagaagaagaactccctcgcctcgcagacgaaggtctcaatcgccgcgtcgcagaagatctcaatctcg |
| | | | | | | | ggaacctcaatgttagtattccttggactcataaggtggggaactttactggtctttattcttctactgtacctgtct |
| | | | | | | | ttaatcctcattggaaaacaccatcttttcctaatatacatttacaccaagacattatcaaaaaatgtgaacagtttg |
| | | | | | | | ttaggcccactacagttaatgagaaaagaagattgcaattgattatgcctgctaggttttatccaaaggttaccaaata |
| | | | | | | | tttaccattggataagggtattaaaccttattatccagaacatctagttaatcattacttccaaactagacactattta |
| | | | | | | | cacactctatggaaggcgggtatattatataagagagaaacaacacatagcgcctcattttgtgggtcaccatattct |
| | | | | | | | tgggaacaagatctacagcatggggcagaatctttccaccagcaatcctctgggattctttcccgaccaccagttggat |
| | | | | | | | ccagccttcagagcaaacacagcaaatccagattgggacttcaatcccaacaaggacacctggccagacgccaacaagg |
| | | | | | | | taggagctggagcattcgggctgggtttcaccccaccgcacggaggccttttggggtggagccctcaggctcagggcat |
| | | | | | | | actacaaactttgccagcaaatccgcctcctgcctccaccaatcgccagacaggaaggcagcctaccccgctgtctc |
| | | | | | | | cacctttgagaaacactcatcctcaggccatgcagtggaattccacaacctttcaccaaactctgcaagatcccaga |
| | | | | | | | gtgagaggcctgtatttccctgctggtggctccagttcaggagcagtaaaccctgttccgactactgcctctcccttat |
| | | | | | | | cgtcaatcttctcgaggattggggaccctgcgctgaacatgagggcagaggaagtctgctaacatgcggtgacgt |
| | | | | | | | cgaggagaatcctggcccaactagt |
| 214 | HBV | sense_5p_HBV_ | sense | L | DNA or | 1625 | agcaccatgcaactttttcacctctgcctaatcatctcttgttcatgtcctactgttcaagcctccaagctgtgccttg |
| | | 41 | | | RNA | | ggtggctttggggcatggacatcgacccttataaagaatttggagctactgtggagttactctcgtttttgccttctga |
| | | | | | | | cttctttccttcagtacgagatcttctagataccgcctcagctctgtatcgggaagccttagagtctcctgagcattgt |
| | | | | | | | tcacctcaccatactgcactcaggcaagcaattctttgctggggggaactaatgactctagctacctgggtgggtgtta |
| | | | | | | | atttggaagatccagcatctagagacctagtagtcagttatgtcaacactaatatgggcctaaagttcaggcaactctt |
| | | | | | | | gtggtttcacatttcttgtctcacttttggaagagaaaccgttatagagtatttggtgtctttcggagtgtggattcgc |
| | | | | | | | actcctccagcttatagaccaccaaatgcccctatcctatcaacacttccggaaactactgttgttagacgacgaggca |
| | | | | | | | ggtcccctagaagaagaactccctcgcctcgcagacgaaggtctcaatcgccgcgtcgcagaagatctcaatctcggga |
| | | | | | | | acctcaatgttagtattccttggactcataaggtggggaactttactggtctttattcttctactgtacctgtctttaa |
| | | | | | | | tcctcattggaaaacaccatcttttcctaatatacatttacaccaagacattatcaaaaaatgtgaacagtttgtaggc |
| | | | | | | | ccacttacagttaatgagaaaagaagattgcaattgattatgcctgctaggttttatccaaaggttaccaaatatttac |
| | | | | | | | cattggataagggtattaaaccttattatccagaacatctagttaatcattacttccaaactagacactatttacacac |
| | | | | | | | tctatggaaggcgggtatattatataagagagaaacaacacatagcgcctcattttgtgggtcaccatattcttgggaa |
| | | | | | | | caagatctacagcatggggcagaatctttccaccagcaatcctctgggattctttcccgaccaccagttggatccagc |
| | | | | | | | cttcagagcaaacacagcaaatccagattgggacttcaatcccaacaaggacactggccagacgccaacaaagtaggag |
| | | | | | | | ctggagcattcgggctgggtttcaccccaccgcacggaggccttttggggtggagccctcaggctcagggcatac |
| | | | | | | | tacaaactttgccagcaaatccgcctcctgcctccaccaatcgccagacaggaaggcagcctaccccgctgtctcc |
| | | | | | | | acctttgagaaacactcatcctcaggccatgcagtggaattccacaacctttcaccaaactctgcaagatcccagag |
| | | | | | | | tgagaggcctgtatttccctgctggtggctccagttcaggagcagtaaaccctgttccgactactgcctctcccttatc |
| | | | | | | | gtcaatcttctcgaggattggggaccctgcgctgaacattcctaacaacttatgagttcactttttttgcaggggaggg |
| | | | | | | | cagaggaagtctgctaacatgcggtgacgtcgaggagaatcctggcccaactagt |
| 215 | HBV | sense_5p_HBV_ | sense | L | DNA or | 315 | aggtttttcacctctgcctaatcatctcttgttcatgtcctactgttcaagcctccaagctgtgccttgggtggctttg |
| | | 60 | | | RNA | | gggcatggacattgacacgtataaagaatttggagcttctgtggagttactctcttttttgccttctgacttctttcct |
| | | | | | | | tctattcaagatctcatcgacaccgcctctgcttaaccaaaatcaacgggactttccaaaatgtcgtaacaactccgcc |
| | | | | | | | ccattgacgcaaatgggcggtaggcgtgtacggtgggaggtctatataagcagagctcgtttagtgaaccgtgccacc |
| 216 | HBV | sense_5p_HBV_ | sense | L | DNA or | 309 | aggtttttcacctctgcctaatcatctcttgttcatgtcctactgttcaagcctccaagctgtgccttgggtggctttg |
| | | 61 | | | RNA | | gcatggacattgacacgtataaagaatttggagcttctgtggagttactctcttttttgccttctgacttctttccttc |
| | | | | | | | tggattcaagatctcatcgacaccgcctctgcttaaccaaaatcaacgggactttccaaaatgtcgtaacaactccgc |
| | | | | | | | cccattgacgcaaatgggcggtaggcgtgtacggtgggaggtctatataagcagagctcgtttagtgaaccgt |
| 217 | HBV | sense_3p_HBV_ | sense | R | DNA or | 1054 | agaaaacttcctattaacaggcctattgattggaaagtatgtcaacgaattgtgggtcttttgggttttgctgccccat |
| | | 21 | | | RNA | | ttacacaatgtggttatcctgcgttaatgcccttgtatgcatgtattcaatctaagcaggctttcactttctcgccaac |
| | | | | | | | ttacaaggcctttctgtgtaaacaatacctgaacctttaccccgttgcccggcaacggccaggtctgtgccaagtgttt |
| | | | | | | | gctgacgcaacccccactggctggggcttggtcatgggccatcagcgcgtgcgtggaaccttttcggctcctctgccg |
| | | | | | | | atccatactgcggaactcctagccgcttgttttgctcgcagcaggtctggagcaaacattatcgggactgataactct |
| | | | | | | | gttgtcctctcccgcaaatatacatcgtatccatggctgctaggctgtgctgccaactggatcctgcgcgggacgtc |
| | | | | | | | ctttgtttacgtcccgtcggcgctgaatcctgcggacgacccttctcggggtcgcttgggactctctcgtccccttctc |
| | | | | | | | cgtctgccgttccgaccgaccacggggcgcacctctctttacgcggactccccgtctgtgccttctcatctgccgga |
| | | | | | | | ccgtgtgcacttcgcttcacctctgcacgtcgcatggagaccaccgtgaacgcccaccgaatgttgcccaaggtct |
| | | | | | | | tacataagaggactcttggactctctgcaatgtcaacgaccgaccttgaggcatacttcaaagactgtttgtttaaaga |
| | | | | | | | ctgggaggagttgggggaggagattagattaaaggtctttgtactaggaggctgtaggcataaattggtctgcgca |
| | | | | | | | ccagcaccatgcaactttttcacctctgcctaatcatctcttgttcatgtcctactgttcaagcctccaagctgtgcct |
| | | | | | | | tgggtggctttggggcatggacatcgacccttataaagaatttggagctacaaaaaaaaaaaaaaaaaaaaaaaaaa |
| | | | | | | | aaaaaaaaaaaaaaaaaaaaaaaaaaaaaaaaaaaaaaaaa |
| 218 | HBV | sense_3p_HBV_ | sense | R | DNA or | 1090 | caagaacacatcatacaaaaaatcaaagaatgttttagaaaacttcctattaacaggcctattgattggaaagtatgtc |
| | | 31 | | | RNA | | aacgaattgtgggtcttttgggttttgctgccccatttacacaatgtggttatcctgcgttaatgcccttgtatgcatg |
| | | | | | | | tattcaatctaagcaggctttcactttctcgccaacttacaaggcctttctgtgtaaacaatacctgaacctttacccc |
| | | | | | | | gttgcccggcaacggccaggtctgtgccaagtgtttgctgacgcaacccccactggctggggcttggtcatgggccatc |
| | | | | | | | agcgcgtgcgtggaaccttttcggctcctctgccgatccatactgcggaactcctagccgcttgttttgctcgcagca |
| | | | | | | | ggtctggagcaaacattatcgggactgataactctgttgtcctctcccgcaaatatacatcgtatccatggctgctag |
| | | | | | | | gctgtgctgccaactggatcctgcgcgggacgtcctttgtttacgtcccgtcggcgctgaatcctgcggacgaccct |
| | | | | | | | tctcggggtcgcttgggactctctcgtccccttctccgtctgccgttccgaccgaccacgggggcacctctctttac |
| | | | | | | | gcggactccccgtctgtgccttctcatctgccggaccgtgtgcacttcgcttcacctctgcacgtcgcatggagacc |
| | | | | | | | accgtgaacgcccaccgaatgttgcccaaggtcttacataagaggactcttggactctctgcaatgtcaacgaccg |
| | | | | | | | accttgaggcatacttcaaagactgtttgtttaaagactgggaggagttgggggaggagattagattaaaggtctttg |
| | | | | | | | tactaggaggctgtaggcataaattggtctgcgcaccagcaccatgcaactttttcacctctgcctaatcatctcttgt |
| | | | | | | | tcatgtcctactgttcaagcctccaagctgtgccttgggtggctttggggcatggacatcgacccttataaagaatttg |
| | | | | | | | gagctacaaaaaaaaaaaaaaaaaaaaaaaaaaaaaaaaaaaaaaaaaaaaaaaaaaaaaaaaaaaaaaaaa |
| | | | | | | | aa |
| 219 | HBV | sense_3p_HBV_ | sense | R | DNA or | 1192 | cttttgtctttgggtatacatttaaaccctaacaaaacaaagagatggggttactctctgaattttatgggttatgtc |
| | | 41 | | | RNA | | attggaagttatgggtccttgccacaagaacacatcatacaaaaaatcaaagaatgttttagaaaacttcctattaaca |
| | | | | | | | ggcctattgattggaaagtatgtcaacgaattgtgggtcttttgggttttgctgccccatttacacaatgtggttatcc |
| | | | | | | | tgcgttaatgcccttgtatgcatgtattcaatctaagcaggctttcactttctcgccaacttacaaggcctttctgtgt |
| | | | | | | | aaacaatacctgaacctttaccccgttgcccggcaacggccaggtctgtgccaagtgtttgctgacgcaacccccact |
| | | | | | | | ggctggggcttggtcatgggccatcagcgcgtgcgtggaaccttttcggctcctctgccgatccatactgcggaactcc |
| | | | | | | | tagccgcttgttttgctcgcagcaggtctggagcaaacattatcgggactgataactctgttgtcctctcccgcaaata |
| | | | | | | | tacatcgtatccatggctgctaggctgtgctgccaactggatcctgcgcgggacgtcctttgtttacgtcccgtcggc |
| | | | | | | | gctgaatcctgcggacgacccttctcggggtcgcttgggactctctcgtccccttctccgtctgccgttccgaccgacc |
| | | | | | | | acggggcgcacctctctttacgcggactccccgtctgtgccttctcatctgccggaccgtgtgcacttcgcttcacctc |
| | | | | | | | tgcacgtcgcatggagaccaccgtgaacgcccaccgaatgttgcccaaggtcttacataagaggactcttggactct |
| | | | | | | | ctgcaatgtcaacgaccgaccttgaggcatacttcaaagactgtttgtttaaagactgggaggagttgggggagga |
| | | | | | | | gattagattaaaggtctttgtactaggaggctgtaggcataaattggtctgcgcaccagcaccatgcaactttttcacc |
| | | | | | | | tctgcctaatcatctcttgttcatgtcctactgttcaagcctccaagctgtgccttgggtggctttggggcatggacat |
| | | | | | | | cgacccttataaagaatttggagctacaaaaaaaaaaaaaaaaaaaaaaaaaaaaaaaaaaaaaaaaaaaaaaaa |
| | | | | | | | aaaaaaaaaaaaaaaaaaa |
| 220 | HBV | sense_3p_HBV_ | sense | R | DNA or | 2609 | ctgttgccctctggtttctccccaggttatccggcgggtttctgacatccggcgggtgactcacaaccccagaaaca |
| | | 60 | | | RNA | | gacatatgcccctatcttatcaacacttccggaaactactgttgttagacgacgaggcaggccccctagaagaaga |
| | | | | | | | actccctcgcctcgcagacgaaggtctcaatcgccgcgtcgcagaagatctcaatctcgggaatctcaatgttagta |
| | | | | | | | tcccttggactcataaggtgggaaactttactgggctttattcttctactgtacctgtctttaatcctgagtggcaaac |
| | | | | | | | tccctcctttcctcacattcatttacaggaggacattattaatagatgtcaacaatatgtgggccctcttacagttaat |
| | | | | | | | gaaaaaaggagattaaaattaattatgcctgctaggttctatcctaaccttaccaaatatttgcccttggacaaaggca |
| | | | | | | | ttaaaccctattatcctgaacatgctgttaatcattacttcaaaactaggcattatttacatactctgtggaaggctgg |
| | | | | | | | cattctatataagagagaaactacacgcagtgcctcattttgtgggtcaccatattcttgggaacaagagctacagcat |
| | | | | | | | gggaggttggtcttccaaacctcgacaaggcatttttcctgctggtggctccagttccggaacagtaaaccctgttccg |
| | | | | | | | actactgcctcacccatatcgtcaatcttctcgaggactggggaccctgcaccgaacatggagaacacaacatcagaat |
| | | | | | | | tcctaggacccctgctcgtgttacaggggggtttttcttgttgacaagaatcctcacaataccacagagtctagactc |
| | | | | | | | gtggtggacttctctcaattttctagggggagcacccgcgtgtcctggccaaaatttgcagtccccaacctccaatca |
| | | | | | | | ctcaccaacctcttgtcctccgatttgacctggctatcgctggatgtgtctgcggcgttttatcatattcctcttcatc |
| | | | | | | | ctgctgctatgcctcatcttcttgttggttcttctggactaccagggtatgttgcccgtttgtcctctacttccagga |
| | | | | | | | acatcaactaccagcacgggaccatgcaagacctgcacgattcctgctcaaggaacctctatgtttccctcttgttgct |
| | | | | | | | gtacaaaaccttcggacggaaactgcacttgtattcccatcccatcatcttgggctttcgcaagattcctatgggagtg |
| | | | | | | | ggcctcagtccgtttctcctgactcagtttactagtgccatttgttcagtggttcgtagggctttcccccactgtttgg |
| | | | | | | | ctttcagttatatggatgatgtggtattgggggccaagtctgtacaacatcttgagtccctttttacctctattaccaa |
| | | | | | | | ttttcttttgtctttgggtatacatttaacccctaataaaaccaaacgttggggctactcccttaacttcatgggatat |
| | | | | | | | gtaattggaagttggggtactttaccgcaagaacatattgtacttaaaatcaaacaatgttttcgaaaactgcctgtaa |
| | | | | | | | atagacctattgattggaaagtatgtcagagaattgtgggtcttttgggctttgctgccccttttacacaatgtggcta |
| | | | | | | | tcctgccttaatgcctttatatgcatgtatacaatctaagcaggctttcactttctcgccaacttacaaggcctttctg |
| | | | | | | | tgtaaacaatatctgaacctttaccccgttgccaggcaacggtcaggtctctgccaagtgtttgctgacgcaaccccc |
| | | | | | | | actggatggggcttggctattggccatcgccgcatgcgtggaacctttgtggctcctctgccgatccatactgcggaac |
| | | | | | | | tcctagcagcttgttttgctcgcagccggtctggagcaaaacttatcggaaccgacaactctgttgtcctctctcggaa |
| | | | | | | | atacacctcctttccatggctgctagggtgtgctgccaactggatcctgcgcgggacgtcctttgtctacgtcccgtcg |
| | | | | | | | gcgctgaatcccgcggacgacccgtctcggggccgtttgggactctatcgtccccttctacatctgccgttccggccga |
| | | | | | | | ccacggggcgcacctctctttacgcggtctccccgtctgtgccttctcatctgccggaccgtgtgcacttcgcttcacc |
| | | | | | | | tctgcacgtcgcatggagaccaccgtgaacgccctccaggtcttgcccaaggtcttacataagaggactctcggactct |
| | | | | | | | cagcaatgtcaacgaccgaccttgaggcgtacttcaaagactgtttgtttaaggactgggaggagttgggggaggagat |
| | | | | | | | taggttaatgatctttgtactaggaggctgtaggcataaattggtctgttcaccagcaccatgcaactttttcacctct |
| | | | | | | | gcctaatcatctcttgttcatgtcctactgttcaagcctccaagctgtgccttgggtggctttggggcatggacatcga |
| | | | | | | | cccttataaagaatttggagcttcaaaaaaaaaaaaaaaaaaaaaaaaaaaaaaaaaaaaaaaaaaaaaaaaaaaaaa |
| | | | | | | | aaaaaaaaaaaaa |
| 221 | HBV | antis_3p_HBV_ | anti- | L | DNA or | 2724 | acggttcactaaacgagctctgcttatatagacctcccaccgtacacgcctaccgcccatttgcgtcaatggggcgg |
| | | 51 | sense | | RNA | | agttgttacgacattttggaaagtcccgttgattttggctgttgccctctggtttctccccaggttatccggcgggtt |
| | | | | | | | tctgacatccggcgggtgactcacaaccccagaaacagacatatgcccctatcttatcaacacttccggaaactactg |
| | | | | | | | ttgttagacgacgaggcaggccccctagaagaagaactccctcgcctcgcagacgaaggtctcaatcgccgcgtc |
| | | | | | | | gcagaagatctcaatctcgggaatctcaatgttagtatcccttggactcataaggtgggaaactttactgggctttatt |
| | | | | | | | cttctactgtacctgtctttaatcctgagtggcaaactccctcctttcctcacattcatttacaggaggacattattaa |
| | | | | | | | tagatgtcaacaatatgtgggccctcttacagttaatgaaaaaaggagattaaaattaattatgcctgctaggttctat |
| | | | | | | | cctaaccttaccaaatatttgcccttggacaaaggcattaaaccctattatcctgaacatgctgttaatcattacttc |
| | | | | | | | aaaactaggcattatttacatactctgtggaaggctggcattctatataagagagaaactacacgcagtgcctcatttt |
| | | | | | | | gtgggtcaccatattcttgggaacaagagctacagcatgggaggttggtcttccaaacctcgacaaggcatttttcctg |
| | | | | | | | ctggtggctccagttccggaacagtaaaccctgttccgactactgcctcacccatatcgtcaatcttctcgaggactgg |
| | | | | | | | ggaccctgcaccgaacatggagaacacaacatcagaattcctaggacccctgctcgtgttacaggcggggtttttcttg |
| | | | | | | | ttgacaagaatcctcacaataccacagagtctagactcgtggtggacttctctcaattttctagggggagcacccgcgt |
| | | | | | | | gtcctggccaaaatttgcagtccccaacctccaatcactcaccaacctcttgtcctccgatttgacctggctatcgctg |
| | | | | | | | gatgtgtctgcggcgttttatcatattcctcttcatcctgctgctatgcctcatcttcttgttggttcttctggactac |
| | | | | | | | cagggtatgttgcccgtttgtcctctacttccaggaacatcaactaccagcacgggaccatgcaagacctgcacgattc |
| | | | | | | | ctgctcaaggaacctctatgtttccctcttgttgctgtacaaaaccttcggacggaaactgcacttgtattcccatccc |
| | | | | | | | atcatcttgggctttcgcaagattcctatgggagtgggcctcagtccgtttctcctgactcagtttactagtgccattt |
| | | | | | | | gttcagtggttcgtagggctttcccccactgtttggctttcagttatatggatgatgtggtattgggggccaagtctgt |
| | | | | | | | acaacatcttgagtccctttttacctctattaccaattttcttttgtctttgggtatacatttaacccctaataaaacc |
| | | | | | | | aaacgttggggctactcccttaacttcatgggatatgtaattggaagttggggtactttaccgcaagaacatattgtac |
| | | | | | | | ttaaaatcaaacaatgttttcgaaaactgcctgtaaatagacctattgattggaaagtatgtcagagaattgtgggtct |
| | | | | | | | tttgggctttgctgccccttttacacaatgtggctatcctgccttaatgcctttatatgcatgtatacaatctaagcag |
| | | | | | | | gctttcactttctcgccaacttacaaggcctttctgtgtaaacaatatctgaacctttaccccgttgccaggcaacggt |
| | | | | | | | caggtctctgccaagtgtttgctgacgcaacccccactggatggggcttggctattggccatcgccgcatgcgtggaac |
| | | | | | | | ctttgtggctcctctgccgatccatactgcggaactcctagcagcttgttttgctcgcagccggtctggagcaaaactt |
| | | | | | | | atcggaaccgacaactctgttgtcctctctcggaaatacacctcctttccatggctgctagggtgtgctgccaactgga |
| | | | | | | | tcctgcgcgggacgtcctttgtctacgtcccgtcggcgctgaatcccgcggacgacccgtctcggggccgtttgggact |
| | | | | | | | ctatcgtccccttctacatctgccgttccggccgaccacggggcgcacctctctttacgcggtctccccgtctgtgcct |
| | | | | | | | tctcatctgccggaccgtgtgcacttcgcttcacctctgcacgtcgcatggagaccaccgtgaacgccctccaggtctt |
| | | | | | | | gcccaaggtcttacataagaggactctcggactctcagcaatgtcaacgaccgaccttgaggcgtacttcaaagactgt |
| | | | | | | | ttgtttaaggactgggaggagttgggggaggagattaggttaatgatctttgtactaggaggctgtaggcataaattgg |
| | | | | | | | tctgttcaccagcaccatgcaactttttcacctctgcctaatcatctcttgttcatgtcctactgttcaagcctccaa |
| | | | | | | | gctgtgccttgggtggctttggggcatggacatcgacccttataaagaatttggagcttcaaaaaaaaaaaaaaaaaa |
| | | | | | | | aaaaaaaaaaaaaaaaaaaaaaaaaaaaaaaaaaaaaaaaaaaaaaaaa |
| 222 | HBV | antis_5p_HBV_ | anti- | L | DNA or | 939 | aactttttcacctctgcctaatcatctcttgttcatgtcctactgttcaagcctccaagctgtgccttgggtggcttt |
| | | 01 | sense | | RNA | | agggcatggacattgacccttataaacaatttggagtgagcaagggcgaggcagtgatcaaggagttcatgcggttca |
| | | | | | | | aggtgcacatggagggctccatgaacggccacgagttcgagatcgagggcgagggcgagggccgcccctacgag |
| | | | | | | | ggcacccagaccgccaagctgaaggtgaccaagggtggccccctgcccttctcctgggacatcctgtcccctca |
| | | | | | | | gttcatgtacggctccagggccttcatcaagcaccccgccgacatccccgactactataagcagtccttccccgag |
| | | | | | | | ggcttcaagtgggagcgcgtgatgaacttcgaggacggcggcgccgtgaccgtgacccaggacacctccctgg |
| | | | | | | | aggacggcaccctgatctacaaggtgaagctccgcggcaccaacttccctcctgacggccccgtaatgcagaag |
| | | | | | | | aagacaatgggctgggaagcgtccaccgagcggttgtaccccgaggacggcgtgctgaagggcgacattaaga |
| | | | | | | | tggccctgcgcctgaaggacggcggccgctacctggcggacttcaagaccacctacaaggccaagaagcccgt |
| | | | | | | | gcagatgcccggcgcctacaacgtcgaccgcaagttggacatcacctcccacaacgaggactacaccgtggtgg |
| | | | | | | | aacagtacgaacgctccgagggccgccactccaccggcggcatggacgagctgtacaagtaacgcgttaagat |
| | | | | | | | acattgatgagtttggacaaaccacaactagaatgcagtgaaaaaaatgctttatttgtgaaatttgtgatgctattg |
| | | | | | | | ctttatttgtaaccattataagctgcaataaacaagttaac |
| 223 | HBV | antis_5p_HBV_ | anti- | L | DNA or | 243 | aactttttcacctctgcctaatcatctcttgttcatgtcctactgttcaagcctccaagctgtgccttgggggcttta |
| | | 20 | sense | | RNA | | gggcatggacatcccttataaacaatttggataacgcgttaagatacattgatgagtttggacaaaccacaactagaat |
| | | | | | | | gcagtgaaaaaaatgctttatttgtgaaatttgtgatgctattgctttatttgtaaccattataagctgcaataaaca |
| | | | | | | | agttaac |
| 224 | HBV | antis_5p_HBV_ | anti- | L | DNA or | 239 | aggtttttcacctctgcctaatcatctcttgttcatgtcctactgttcaagcctccaagctgtgccttgggtggcttt |
| | | 50 | sense | | RNA | | ggggcatggacattgacacgtataaagaatttggagcttctgtggagttactctcttttttgccttctgacttctttc |
| | | | | | | | cttctattcaagatctcatcgacaccgcctctgcttaataagacaaaccacaactagaatgcagtgaaaaaaatgctt |
| | | | | | | | tattt |
| 225 | HBV | antis_3p_HBV_ | anti- | R | DNA or | 1323 | ggtaccgtcgactgcagaattcgaagcttgagctcgagatctgagtccggtagcgctagcggatctgacggttcac |
| | | 01 | sense | | RNA | | taaaccagctctgcttatatagacctcccaccgtacacgcctaccgcccatttgcgtcaatggggcggagttgttac |
| | | | | | | | gacattttggaaagtcccgttgattttggtgccaaaacaaactcccattgacgtcaatggggtggagacttggaaatc |
| | | | | | | | cccgtgagtcaaaccgctatccacgcccattgatgtactgccaaaaccgcatcaccatggtaatagcgatgactaat |
| | | | | | | | acgtagatgtactgccaagtaggaaagtcccataaggtcatgtactgggcataatgccaggcgggccatttaccgt |
| | | | | | | | cattgacgtcaatagggggcgtacttggcatatgatacacttgatgtactgccaagtgggcagtttaccgtaaatact |
| | | | | | | | ccacccattgacgtcaatggaaagtccctattggcgttactatgggaacatacgtcattattgacgtcaatgggcgg |
| | | | | | | | gggtcgttgggcggtcagccaggcgggccatttaccgtaagttatgtaacgactagtaggcaatcagcgcatgcg |
| | | | | | | | tggaaccattgtggctcctctgccgatccatactgcggaactcctagccgcttgttttgctcgcagccggtctgggg |
| | | | | | | | caaagctcatcggaactgacaattctgttgtcctctcgcggaaatatacatcgtttccatggctgctaggctgtgctgc |
| | | | | | | | caactggatcctgcgcgggacgtcctttgtttacgtcccgtcggcgctgaatcctgcggacgacccttctcggggtc |
| | | | | | | | gcttgggactctctcgtccccttctccgtctgccgttccgaccgaccacggggcgcacctctctttacgcggactcc |
| | | | | | | | ccgtctgtgccttctcatctgccggaccgtgtgcacttcgcttcacctctgcacgtcgcatggagaccaccgtgaac |
| | | | | | | | gcccaccgaatgttgcccaaggtcttacataagaggactcttggactctctgcaatgtcaacgaccgaccttgagg |
| | | | | | | | catacttcaaagactgtttgtttaaagactgggaggagttgggggaggagattagattaaaggtctttgtactaggag |
| | | | | | | | gctgtaggcataaattggtctgcgcaccagcaccatgcaactttttcacctctgcctaatcatctcttgttcatgtcct |
| | | | | | | | actgttcaagcctccaagctgtgccttgggtggctttggggcatggacatcgacccttataaacaatttggataacgat |
| | | | | | | | cgtaaaacta |
| 226 | HBV | antis_3p_HBV_ | anti- | R | DNA or | 2730 | ggtggcacggttcactaaacgagctctgcttatatagacctcccaccgtacacgcctaccgcccatttgcgtcaatg |
| | | 50 | sense | | RNA | | gggcggagttgttacgacattttggaaagtcccgttgattttggctgttgccctctggtttctccccaggttatccggc |
| | | | | | | | gggtttctgacatccgggggtgactcacaaccccagaaacagacatatgcccctatcttatcaacacttccggaa |
| | | | | | | | actactgttgttagacgacgaggcaggccccctagaagaagaactccctcgcctcgcagacgaaggtctcaatcg |
| | | | | | | | ccgcgtcgcagaagatctcaatctcgggaatctcaatgttagtatcccttggactcataaggtgggaaactttactgg |
| | | | | | | | gctttattcttctactgtacctgtctttaatcctgagtggcaaactccctcctttcctcacattcatttacaggaggac |
| | | | | | | | attattaatagatgtcaacaatatgtgggccctcttacagttaatgaaaaaaggagattaaaattaattatgcctgct |
| | | | | | | | aggttctatcctaaccttaccaaatatttgcccttggacaaaggcattaaaccctattatcctgaacatgctgttaatc |
| | | | | | | | attacttcaaaactaggcattatttacatactctgtggaaggctggcattctatataagagagaaactacacgcagtgc |
| | | | | | | | ctcattttgtgggtcaccatattcttgggaacaagagctacagcatgggaggttggtcttccaaacctcgacaaggcat |
| | | | | | | | ttttcctgctggtggctccagttccggaacagtaaaccctgttccgactactgcctcacccatatcgtcaatcttctcg |
| | | | | | | | aggactggggaccctgcaccgaacatggagaacacaacatcagaattcctaggacccctgctcgtgttacaggcggggt |
| | | | | | | | ttttcttgttgacaagaatcctcacaataccacagagtctagactcgtggtggacttctctcaattttctagggggagc |
| | | | | | | | acccgcgtgtcctggccaaaatttgcagtccccaacctccaatcactcaccaacctcttgtcctccgatttgacctggc |
| | | | | | | | tatcgctggatgtgtctgcggcgttttatcatattcctcttcatcctgctgctatgcctcatcttcttgttggttcttc |
| | | | | | | | tggactaccagggtatgttgcccgtttgtcctctacttccaggaacatcaactaccagcacgggaccatgcaagacctg |
| | | | | | | | cacgattcctgctcaaggaacctctatgtttccctcttgttgctgtacaaaaccttcggacggaaactgcacttgtatt |
| | | | | | | | cccatcccatcatcttgggctttcgcaagattcctatgggagtgggcctcagtccgtttctcctgactcagtttactag |
| | | | | | | | tgccatttgttcagtggttcgtagggctttcccccactgtttggctttcagttatatggatgatgtggtattgggggcc |
| | | | | | | | aagtctgtacaacatcttgagtccctttttacctctattaccaattttcttttgtctttgggtatacatttaaccccta |
| | | | | | | | ataaaaccaaacgttggggctactcccttaacttcatgggatatgtaattggaagttggggtactttaccgcaagaac |
| | | | | | | | atattgtacttaaaatcaaacaatgttttcgaaaactgcctgtaaatagacctattgattggaaagtatgtcagagaat |
| | | | | | | | tgtgggtcttttgggctttgctgccccttttacacaatgtggctatcctgccttaatgcctttatatgcatgtatacaa |
| | | | | | | | tctaagcaggctttcactttctcgccaacttacaaggcctttctgtgtaaacaatatctgaacctttaccccgttgcca |
| | | | | | | | ggcaacggtcaggtctctgccaagtgtttgctgacgcaacccccactggatggggcttggctattggccatcgccgcat |
| | | | | | | | gcgtggaacctttgtggctcctctgccgatccatactgcggaactcctagcagcttgttttgctcgcagccggtctgga |
| | | | | | | | gcaaaacttatcggaaccgacaactctgttgtcctctctcggaaatacacctcctttccatggctgctagggtgtgctg |
| | | | | | | | ccaactggatcctgcgcgggacgtcctttgtctacgtcccgtcggcgctgaatcccgcggacgacccgtctcggggccg |
| | | | | | | | tttgggactctatcgtccccttctacatctgccgttccggccgaccacggggcgcacctctctttacgcggtctcccc |
| | | | | | | | gtctgtgccttctcatctgccggaccgtgtgcacttcgcttcacctctgcacgtcgcatggagaccaccgtgaacgccc |
| | | | | | | | tccaggtcttgcccaaggtcttacataagaggactctcggactctcagcaatgtcaacgaccgaccttgaggcgtactt |
| | | | | | | | caaagactgtttgtttaaggactgggaggagttgggggaggagattaggttaatgatctttgtactaggaggctgtag |
| | | | | | | | gcataaattggtctgttcaccagcaccatgcaactttttcacctctgcctaatcatctcttgttcatgtcctactgtt |
| | | | | | | | caagcctccaagctgtgccttgggtggctttggggcatggacatcgacccttataaagaatttggagcttcaaaaaaa |
| | | | | | | | aaaaaaaaaaaaaaaaaaaaaaaaaaaaaaaaaaaaaaaaaaaaaaaaaaaaaaaaaaaa |
| 227 | ZEBOV | antis_5p_ZEBOV | anti- | L | DNA or | 740 | tggacacacaaaaaagaagaaatagatttatttttaaatttttgtgtgcgaccatttttccaggaatcctttttgcaac |
| | | | sense | | RNA | | gtttattctggcgagccttctgatgagcgtggtcaatgtcttaattatcattaacacgaagattattatctagtctgct |
| | | | | | | | atattgtaaagaaggtatttctgattttacagtcctgccttttcttttaattttatcattctgtatgaaatagtagctt |
| | | | | | | | actagacaaaaatgttaaggttattacacctaatctcttattttacacaaagattaaggctatcaccgtttttaaaacg |
| | | | | | | | acaaattaaagtaatgataattataaaaaacttaatcttaaagatcaattttgttttaagaagttgtgttatcaaccaa |
| | | | | | | | aacactattccatctgacaggatattgatacaacattattatatcaataacaatatgagcccagacctttcgttaaagc |
| | | | | | | | ttaattataaagagtgcagagtttattatgttgcgttaaattcattgcggcagtaatatgtcacacatgctgcattgtg |
| | | | | | | | aattttttgattatcacgcagggagagaggctaaatatagtatataatatagggactcctttcgttatttaaatagttg |
| | | | | | | | tttattgagattagatcaatatgatgtatgagagcaatttatgagttttttataatctgtatgttaataaccaaccttt |
| | | | | | | | gcaagtatcaggatactatgcacggtaat |
| 228 | ZEBOV | antis_5p_ | anti- | L | DNA or | 743 | aggtggacacacaaaaaagaagaaatagatttatttttaaatttttgtgtgcgaccatttttccaggaatcctttttgc |
| | | ZEBOV_add_agg | sense | | RNA | | aacgtttattctggcgagccttctgatgagcgtggtcaatgtcttaattatcattaacacgaagattattatctagtct |
| | | | | | | | gctatattgtaaagaaggtatttctgattttacagtcctgccttttcttttaattttatcattctgtatgaaatagtag |
| | | | | | | | cttactagacaaaaatgttaaggttattacacctaatctcttattttacacaaagattaaggctatcaccgtttttaaa |
| | | | | | | | acgacaaattaaagtaatgataattataaaaaacttaatcttaaagatcaattttgttttaagaagttgtgttatcaac |
| | | | | | | | caaaacactattccatctgacaggatattgatacaacattattatatcaataacaatatgagcccagacctttcgttaa |
| | | | | | | | agcttaattataaagagtgcagagtttattatgttgcgttaaattcattgcggcagtaatatgtcacacatgctgcat |
| | | | | | | | tgtgaattttttgattatcacgcagggagagaggctaaatatagtatataatatagggactcctttcgttatttaaata |
| | | | | | | | gttgtttattgagattagatcaatatgatgtatgagagcaatttatgagttttttataatctgtatgttaataaccaac |
| | | | | | | | ctttgcaagtatcaggatactatgcacggtaat |
| 229 | ZEBOV | antis_5p_ | anti- | L | DNA or | 737 | tggacacacaaaaaagaagaaatagatttatttttaaatttttgtgtgcgaccatttttccaggaatcctttttgcaac |
| | | ZEBOV_Δ3 | sense | | RNA | | gtttattctggcgagccttctgatgagcgtggtcaatgtcttaattatcattaacacgaagattattatctagtctgct |
| | | | | | | | atattgtaaagaaggtatttctgattttacagtcctgccttttcttttaattttatcattctgtatgaaatagtagctt |
| | | | | | | | actagacaaaaatgttaaggttattacacctaatctcttattttacacaaagattaaggctatcaccgtttttaaaacg |
| | | | | | | | acaaattaaagtaatgataattataaaaaacttaatcttaaagatcaattttgttttaagaagttgtgttatcaaccaa |
| | | | | | | | aacactattccatctgacaggatattgatacaacattattatatcaataacaatatgagcccagacctttcgttaaagc |
| | | | | | | | ttaattataaagagtgcagagtttattatgttgcgttaaattcattgcggcagtaatatgtcacacatgctgcattgt |
| | | | | | | | gaattttttgattatcacgcagggagagaggctaaatatagtatataatatagggactcctttcgttatttaaatagt |
| | | | | | | | tgtttattgagattagatcaatatgatgtatgagagcaatttatgagttttttataatctgtatgttaataaccaac |
| | | | | | | | ctttgcaagtatcaggatactatgcacggt |
| 230 | ZEBOV | antis_5p_ | anti- | L | DNA or | 737 | aggacacacaaaaaagaagaaatagatttatttttaaatttttgtgtgcgaccatttttccaggaatcctttttgcaac |
| | | ZEBOV_Δ3_ag | sense | | RNA | | gtttattctggcgagccttctgatgagcgtggtcaatgtcttaattatcattaacacgaagattattatctagtctgc |
| | | | | | | | tatattgtaaagaaggtatttctgattttacagtcctgccttttcttttaattttatcattctgtatgaaatagtagct |
| | | | | | | | tactagacaaaaatgttaaggttattacacctaatctcttattttacacaaagattaaggctatcaccgtttttaaaac |
| | | | | | | | gacaaattaaagtaatgataattataaaaaacttaatcttaaagatcaattttgttttaagaagttgtgttatcaacca |
| | | | | | | | aaacactattccatctgacaggatattgatacaacattattatatcaataacaatatgagcccagacctttcgttaaag |
| | | | | | | | cttaattataaagagtgcagagtttattatgttgcgttaaattcattgcggcagtaatatgtcacacatgctgcattgt |
| | | | | | | | gaattttttgattatcacgcagggagagaggctaaatatagtatataatatagggactcctttcgttatttaaatagtt |
| | | | | | | | gtttattgagattagatcaatatgatgtatgagagcaatttatgagttttttataatctgtatgttaataaccaacct |
| | | | | | | | ttgcaagtatcaggatactatgcacggt |
| 231 | ZEBOV | antis_3p_ZEBOV | anti- | R | DNA or | 469 | actcggaattttgtgattccgagcaatttgtcttttaactatttccaatgttttaccccaagctttagggttgttgaat |
| | | | sense | | RNA | | cttcaatcccctaagtgttcaagttcaaaaccaaggttaattctcaatcttgcttgtttgactgtgaactaatgctgtc |
| | | | | | | | ctttaacaataatgtttctattaaggttgttggatcaccctgcagagtctgaggtggaacttgtgatgtgataagacct |
| | | | | | | | aggtgttgacaagggattttcaactgagcacactgatgcccttgctcccttcggagacctaggttgttctctatctttg |
| | | | | | | | tgatgtggctctgaaacaaaccaggtgtgattacagtaacaatttcaatttaaattccgatataaatttcaatgagagg |
| | | | | | | | aaaattattaatcttcctcatagttattcgcacacaaaagatcctaaaaattcttcttttttttgtgtgtccg |
| 232 | SEBOV | antis_5p_SEBOV | anti- | L | DNA or | 708 | tggacacacaaaaaagagaaaatacagattttttaatttatttgtgtgcgcttaattttcctggtgaggttaagatctt |
| | | | sense | | RNA | | cctgcacctctaatgtaacttttttcttactactaattaaatcattgacattgagattaaaaacaaaaaaatgtcgtaa |
| | | | | | | | gaacatcttatggaatttgtagattatcaatcaactacctggttggaataaaaggttcattgaccgtgttcttcttatg |
| | | | | | | | ttattcccaccggccctaggaaatattttggctgtataagtattaaacttagctttaaaagtgaaattgcaatttagta |
| | | | | | | | tcagaaataacctgtcatgtgaaacatattactagttcttagcataagagtattagtcttagtaattttttcttaatac |
| | | | | | | | ttatgtaactggagtcaacctagcttctaaatttgaatttaatatatgaactagggcaacaatttggttgtgatgttcg |
| | | | | | | | ttatagatttgtttattttagtcataatagctaggaaaaattattatttacattatcaacataatcaatagagtgtctt |
| | | | | | | | cgaaaccttgaatcgatgcttccaacgctgtatattggaagagtcaataaactaatgccaaatgtggtcccttgcatat |
| | | | | | | | tgttcttacacttgtattgactttgatatatacctggttatttaaaatacttctagaacctgagatgcagttagaa |
| 233 | SEBOV | antis_5p_ | anti- | L | DNA or | 711 | aggtggacacacaaaaaagagaaaatacagattttttaatttatttgtgtgcgcttaattttcctggtgaggttaagat |
| | | SEBOV_add_agg | sense | | RNA | | cttcctgcacctctaatgtaacttttttcttactactaattaaatcattgacattgagattaaaaaaaaaaaatgtcgt |
| | | | | | | | aagaacatcttatggaatttgtagattatcaatcaactacctggttggaataaaaggttcattgaccgtgttcttctta |
| | | | | | | | tgttattcccaccggccctaggaaatattttggctgtataagtattaaacttagctttaaaagtgaaattgcaattta |
| | | | | | | | gtatcagaaataacctgtcatgtgaaacatattactagttcttagcataagagtattagtcttagtaattttttcttaa |
| | | | | | | | tacttatgtaactggagtcaacctagcttctaaatttgaatttaatatatgaactagggcaacaatttggttgtgatgt |
| | | | | | | | tcgttatagatttgtttattttagtcataatagctaggaaaaattattatttacattatcaacataatcaatagagtgt |
| | | | | | | | cttcgaaaccttgaatcgatgcttccaacgctgtatattggaagagtcaataaactaatgccaaatgtggtcccttgca |
| | | | | | | | tattgttcttacacttgtattgactttgatatatacctggttatttaaaatacttctagaacctgagatgcagttagaa |
| 234 | SEBOV | antis_5p_ | anti- | L | DNA or | 705 | tggacacacaaaaaagagaaaatacagattttttaatttatttgtgtgcgcttaattttcctggtgaggttaagatctt |
| | | SEBOV_Δ3 | sense | | RNA | | cctgcacctctaatgtaacttttttcttactactaattaaatcattgacattgagattaaaaaaaaaaaatgtcgtaag |
| | | | | | | | aacatcttatggaatttgtagattatcaatcaactacctggttggaataaaaggttcattgaccgtgttcttcttatgt |
| | | | | | | | tattcccaccggccctaggaaatattttggctgtataagtattaaacttagctttaaaagtgaaattgcaatttagtat |
| | | | | | | | cagaaataacctgtcatgtgaaacatattactagttcttagcataagagtattagtcttagtaattttttcttaatact |
| | | | | | | | tatgtaactggagtcaacctagcttctaaatttgaatttaatatatgaactagggcaacaatttggttgtgatgttcgt |
| | | | | | | | tatagatttgtttattttagtcataatagctaggaaaaattattatttacattatcaacataatcaatagagtgtcttc |
| | | | | | | | gaaaccttgaatcgatgcttccaacgctgtatattggaagagtcaataaactaatgccaaatgtggtcccttgcatat |
| | | | | | | | tgttcttacacttgtattgactttgatatatacctggttatttaaaatacttctagaacctgagatgcagtta |
| 235 | SEBOV | antis_5p_ | anti- | L | DNA or | 705 | aggacacacaaaaaagagaaaatacagattttttaatttatttgtgtgcgcttaattttcctggtgaggttaagatctt |
| | | SEBOV_Δ3_ag | sense | | RNA | | cctgcacctctaatgtaacttttttcttactactaattaaatcattgacattgagattaaaaacaaaaaaatgtcgtaa |
| | | | | | | | gaacatcttatggaatttgtagattatcaatcaactacctggttggaataaaaggttcattgaccgtgttcttcttatg |
| | | | | | | | ttattcccaccggccctaggaaatattttggctgtataagtattaaacttagctttaaaagtgaaattgcaatttagta |
| | | | | | | | tcagaaataacctgtcatgtgaaacatattactagttcttagcataagagtattagtcttagtaattttttcttaata |
| | | | | | | | cttatgtaactggagtcaacctagcttctaaatttgaatttaatatatgaactagggcaacaatttggttgtgatgttc |
| | | | | | | | gttatagatttgtttattttagtcataatagctaggaaaaattattatttacattatcaacataatcaatagagtgtct |
| | | | | | | | tcgaaaccttgaatcgatgcttccaacgctgtatattggaagagtcaataaactaatgccaaatgtggtcccttgcata |
| | | | | | | | ttgttcttacacttgtattgactttgatatatacctggttatttaaaatacttctagaacctgagatgcagtta |
| 236 | SEBOV | antis_3p_SEBOV | anti- | R | DNA or | 457 | gttgtaggctctcaggtaaacccttagtagatagcgtgtgatgaaggtaatcagtccacttagctaaaattggctaga |
| | | | sense | | RNA | | ttttgtggactaaagggatacctcggggccctcctctcaccctctcttgagacttgggatagtatcggtcttactagaa |
| | | | | | | | aatgctaactaccaggacaaatttagtctattactctggtaaccacacttatcaagaaggttagaattgtaggaggag |
| | | | | | | | ccaggttaggaaaaggccaagcaatacgacccacataccttcgcaacactttaggaatgcccccaaatgggggat |
| | | | | | | | taactagttaaattgtctccaattggtaaatgatgagatcaatctcaatgtcaatattagtttgagtttgaggaaaatg |
| | | | | | | | attaatcttcctcatagttattcgcacacaaaaaatcttaaaaacttttctttctttttgtgtgtccg |
| 237 | MARV | antis_5p_MARV | anti- | L | DNA or | 635 | ggacacacaaaaaagatgaagaatgttttgtgttacttatatcaaagctttttaattttttaattttatatattggttt |
| | | | sense | | RNA | | ttcttaatttacattcacatcgacattctagaaatttaactacgaagttttatcacttgtcatcagagaaaacatgacg |
| | | | | | | | tggtttttttgatctcaacggtaacttaattcttttcctaatgtatttaaaagaggtgcttgtagattacagcagacac |
| | | | | | | | ttttttatatttatgatatatggtatgtcataattcaattcaactagtgtatctcagtatttccttaatttcttacacc |
| | | | | | | | atattagggataaagctcaaggattcacccaatgtgccaatattaatgctaattatgagcaataacatctgagtaatta |
| | | | | | | | acgttaaaaagttacctctggtgtgaccaaacactaatcactagttaatctgtgcgcgtaattttctcacttgtgtgag |
| | | | | | | | gcctaaagcgatataagattatcttgtataaaataagctgtctaatagtcttttcgttttcttatttattttattact |
| | | | | | | | tctcatacaatatcttagacaatgtaaccctcactcttaaatagattgtttttgtgaattaaatgaagttctctctgt |
| | | | | | | | tgaaa |
| 238 | MARV | antis_5p_MARV_ | anti- | L | DNA or | 636 | aggacacacaaaaaagatgaagaatgttttgtgttacttatatcaaagctttttaattttttaattttatatattggtt |
| | | add_a | sense | | RNA | | tttcttaatttacattcacatcgacattctagaaatttaactacgaagttttatcacttgtcatcagagaaaacatgac |
| | | | | | | | gtggtttttttgatctcaacggtaacttaattcttttcctaatgtatttaaaagaggtgcttgtagattacagcagaca |
| | | | | | | | cttttttatatttatgatatatggtatgtcataattcaattcaactagtgtatctcagtatttccttaatttcttacac |
| | | | | | | | catattagggataaagctcaaggattcacccaatgtgccaatattaatgctaattatgagcaataacatctgagtaat |
| | | | | | | | taacgttaaaaagttacctctggtgtgaccaaacactaatcactagttaatctgtgcgcgtaattttctcacttgtgtg |
| | | | | | | | aggcctaaagcgatataagattatcttgtataaaataagctgtctaatagtcttttcgttttcttatttattttattac |
| | | | | | | | ttctcatacaatatcttagacaatgtaaccctcactcttaaatagattgtttttgtgaattaaatgaagttctctctg |
| | | | | | | | ttgaaa |
| 239 | MARV | antis_5p_MARV_ | anti- | L | DNA or | 635 | agacacacaaaaaagatgaagaatgttttgtgttacttatatcaaagctttttaattttttaattttatatattggttt |
| | | gla | sense | | RNA | | ttcttaatttacattcacatcgacattctagaaatttaactacgaagttttatcacttgtcatcagagaaaacatgacg |
| | | | | | | | tggtttttttgatctcaacggtaacttaattcttttcctaatgtatttaaaagaggtgcttgtagattacagcagacac |
| | | | | | | | ttttttatatttatgatatatggtatgtcataattcaattcaactagtgtatctcagtatttccttaatttcttacacc |
| | | | | | | | atattagggataaagctcaaggattcacccaatgtgccaatattaatgctaattatgagcaataacatctgagtaatta |
| | | | | | | | acgttaaaaagttacctctggtgtgaccaaacactaatcactagttaatctgtgcgcgtaattttctcacttgtgtgag |
| | | | | | | | gcctaaagcgatataagattatcttgtataaaataagctgtctaatagtcttttcgttttcttatttattttattact |
| | | | | | | | tctcatacaatatcttagacaatgtaaccctcactcttaaatagattgtttttgtgaattaaatgaagttctctctgt |
| | | | | | | | tgaaa |
| 240 | MARV | antis_3p_MARV | anti- | R | DNA or | 103 | ctcttcaagaatattaacagactgacaagtctcaatgtcaatgttaatattcttctttatttatatgatacacaaaat |
| | | | sense | | RNA | | catcatctcttgtttttgtgtgtct |
| 241 | RV | antis_5p_RV | anti- | L | DNA or | 49 | ataaaactaaaaaactaattgtttattatttctatatctaaacaattta |
| | | | sense | | RNA | | |
| 242 | RV | antis_3p_RV | anti- | R | DNA or | 613 | ggtgtcaatatataatactatatattgtcaccataagcaatcaataaggataaaaaggaaacacggacacccaaagt |
| | | | sense | | RNA | | agttggtcccatcccgcaattgctcattacgactagatacatactggattgcatgcactagctgcagggttaaggtta |
| | | | | | | | gccacattcaggggccggaggactcaaagtgagcacacggggctcttcacaccttgtccattatatggcttcacac |
| | | | | | | | ccagagggtgtgcaggcagccacgcaggctagaacactgtcgccagtggggtacttctagcctcatctgccaggt |
| | | | | | | | ctaccatggggtttggctgagcgaccaaccacgtaacttcaaaagtgttactaagctttgcgtaggcactttgcgggt |
| | | | | | | | aacgatctcagctgtttttgccctgttggagcatatcagctttgaccggggaaacagaagtgcttgacctttgacaatc |
| | | | | | | | tgggagattgccgcctattggtcattttgtttaacttctaagttacaaaagggggaggggatcataaaactggcgtac |
| | | | | | | | aaaattaccgtaataatagtgtactactcccgttaggagatccaggtgggaacaacctggatccagttttaa |
| 243 | EVD68 | antis_5p_EVD68 | anti- | L | DNA or | 65 | ccaagtgaccaaaatttacctctaatgaaagtgactataacttgggtctcaattagagttgtcta |
| | | | sense | | RNA | | |
| 244 | EVD68 | antis_3p_EVD68 | anti- | R | DNA or | 697 | tatgaaagttttcaaatcaaggttatcaaaatgcaaattggtaagtaagacttaggatcttttgcaattaatttgaca |
| | | | sense | | RNA | | gctaaatggtaacaatatcaaattgtcaccataagcagtcataaaagtaaaaagtgaaacacggacacccaaagtagtc |
| | | | | | | | ggttccgccacggacttgcgcattacgacaagcaactcactggcttgtgagcacttgctccatggttaggattagcc |
| | | | | | | | gcattcaggggccggaggactctatagtagctcaatagactcttcacaccttgttcatgtctagcgtctcatggttttc |
| | | | | | | | accatgagtaggccgccaacgcagcctggaccactgtcgccagtggggtgtgtccagactcatcgacccaagct |
| | | | | | | | acacacgggttagtgtgctgagcgcaacgcgtcaacaatccaaaggtgatactaggtttctcgaagtactatagcg |
| | | | | | | | gataacggataggttgttttcaaccgtgggaacagtttatacaaccacaccggggaaacagaagtgcttgctcataa |
| | | | | | | | gagcgctggtttgcgccccacctattgagccttgatttaaaagcttctaaattacattgggaagggagataaaacag |
| | | | | | | | gcgtacaaaggtaccgaagtaccagagtactagccgccacgtgggcccttggagtgggaacaaccccaaagct |
| | | | | | | | gttttaa |
| 245 | NDV | antis_5p_NDV | anti- | L | DNA or | 191 | accaaacaaagatttggtgaatgacaagactacactcaagaataattgtgcgcaacctttttttaagacatttatttg |
| | | | sense | | RNA | | agttcgaattcgagctctaaggagtcagagttcaacttttttctaacatagtataattaaatcaccaaggatacaatt |
| | | | | | | | ggccagaaaaggagcctattaatatgtgattttcg |
| 246 | NDV | antis_5p_NDV_ | anti- | L | DNA or | 191 | aggaccaaacaaagatttggtgaatgacaagactacactcaagaataattgtgcgcaacctttttttaagacatttat |
| | | Δ3_add_agg | sense | | RNA | | ttgagttcgaattcgagctctaaggagtcagagttcaacttttttctaacatagtataattaaatcaccaaggataca |
| | | | | | | | attggccagaaaaggagcctattaatatgtgattt |
| 247 | NDV | antis_5p_NDV_ | anti- | L | DNA or | 191 | agcaaacaaagatttggtgaatgacaagactacactcaagaataattgtgcgcaacctttttttaagacatttatttg |
| | | ag | sense | | RNA | | agttcgaattcgagctctaaggagtcagagttcaacttttttctaacatagtataattaaatcaccaaggatacaatt |
| | | | | | | | ggccagaaaaggagcctattaatatgtgattttcg |
| 248 | NDV | antis_3p_NDV | anti- | L | DNA or | 121 | accaaacaaagatttggtgaatgacaagactacactcaagaataattgtgcgcaacctttttttaagacatttatttg |
| | | | sense | | RNA | | agttcgaattcgagctctaaggagtcagagttcaacttttttctaacatagtataattaaatcaccaaggatacaattg |
| | | | | | | | gccagaaaaggagcctattaatatgtgattttcg |
| 249 | EEEV | antis_5p_EEEV | anti- | L | DNA or | 374 | tttttttttttttttgaaatattaaaaacaaaataaagacataaaagaaaaatttaatatgattttgtaaattgatat |
| | | | sense | | RNA | | aaaagacagcattatgcgctgcctgtagtgtttgtaagaggcggtatattacctgtagtgtaattgtatatacggcaat |
| | | | | | | | tatgcctgattataaagtgtgtataaaattacttaggagcagcattatgcactgcccttagtgttataatttggtaata |
| | | | | | | | tagcagccttatgcactgcatcggcggcattatgcaccaccgtcaccatagctcggaagtacatcgaactagggtgaat |
| | | | | | | | ttagtgtttagtaagacagccttatacactgcctgtagtgttatgtggttgacaagatg |
| 250 | EEEV | antis_3p_EEEV | anti- | R | DNA or | 283 | ctctgatatttaaatagcaatataatataaataggttaggtactaatgcacaacctatttaggtcagccgtagagggtt |
| | | | sense | | RNA | | atggggtgacctcttatagctaggtgcgaaaacgccctagcattagcatggtcattgtcagtgacctgcgttgcttcta |
| | | | | | | | tctcaaaatgtggaaagcatctttgcagtgacttgacgaatgggctgtctgcgtctaagtcaacatgaactttctccat |
| | | | | | | | tttggataggtggaaatagggtggttgcctctacaccgtaccctat |
| 251 | EEEV | antis_3p_EEEV_ | anti- | R | DNA or | 475 | tgggccaggattctcctcgacgtcaccgcatgttagcagacttcctctgccctctccactgccagctaggtgcgaaa |
| | | V2 | sense | | RNA | | acgccctagcattagcatggtcattgtcagtgacctgcgttgcttctatctcaaaatgtggaaagcatctttgcagtga |
| | | | | | | | cttgacgaatgggctgtctgcgtctaagtcaacatgaactttctccattttggataggtggaaatagggtggttgcctc |
| | | | | | | | tacaccgtaccctatttaggtcagccgtagagggttatggggtgacctcttatagctaggtgcgaaaacgccctagc |
| | | | | | | | attagcatggtcattgtcagtgacctgcgttgcttctatctcaaaatgtggaaagcatctttgcagtgacttgacgaat |
| | | | | | | | gggctgtctgcgtctaagtcaacatgaactttctccattttggataggtggaaatagggtggttgcctctacaccgtac |
| | | | | | | | cctat |
| 252 | EEEV | sense_5p_EEEV | sense | L | DNA or | 283 | atagggtacggtgtagaggcaaccaccctatttccacctatccaaaatggagaaagttcatgttgacttagacgcag |
| | | | | | RNA | | acagcccattcgtcaagtcactgcaaagatgctttccacattttgagatagaagcaacgcaggtcactgacaatgac |
| | | | | | | | catgctaatgctagggcgttttcgcacctagctataagaggtcaccccataaccctctacggctgacctaaataggtt |
| | | | | | | | gtgcattagtacctaacctatttatattatattgctatttaaatatcagag |
| 253 | EEEV | Sense_5p_EEEV_ | sense | L | DNA or | 475 | atagggtacggtgtagaggcaaccaccctatttccacctatccaaaatggagaaagttcatgttgacttagacgcag |
| | | V2 | | | RNA | | acagcccattcgtcaagtcactgcaaagatgctttccacattttgagatagaagcaacgcaggtcactgacaatgac |
| | | | | | | | catgctaatgctagggcgttttcgcacctagctataagaggtcaccccataaccctctacggctgacctaaataggg |
| | | | | | | | tacggtgtagaggcaaccaccctatttccacctatccaaaatggagaaagttcatgttgacttagacgcagacagcc |
| | | | | | | | cattcgtcaagtcactgcaaagatgctttccacattttgagatagaagcaacgcaggtcactgacaatgaccatgcta |
| | | | | | | | atgctagggcgttttcgcacctagctggcagtggagagggcagaggaagtctgctaacatgcggtgacgtcgag |
| | | | | | | | gagaatcctggccca |
| 254 | EEEV | sense_3p_EEEV | sense | R | DNA or | 381 | catcttgtcaaccacataacactacaggcagtgtataaggctgtcttactaaacactaaattcaccctagttcgatgta |
| | | | | | RNA | | cttccgagctatggtgacggtggtgcataatgccgccgatgcagtgcataaggctgctatattaccaaattataaca |
| | | | | | | | ctaagggcagtgcataatgctgctcctaagtaattttatacacactttataatcaggcataattgccgtatatacaatt |
| | | | | | | | acactacaggtaatataccgcctcttacaaacactacaggcagcgcataatgctgtcttttatatcaatttacaaaatc |
| | | | | | | | atattaaatttttcttttatgtctttattttgtttttaatatttcaaaaaaaaaaaaaaaaaaaaaa |
| 255 | VEEV | antis_5p_VEEV | anti- | L | DNA or | 135 | tttttttttttttttgaaatattaaaaacaaaatccgattcggaaaagaaaagaaaaaataaaataaaaattttaagg |
| | | | sense | | RNA | | cggcatgccaatcgccgcgagttctatgtaagcagcttgccaattgctgctgtattc |
| 256 | VEEV | antis_3p_VEEV | anti- | R | DNA or | 373 | cttggcggactagactatgtcgtagtccattcaggttagccgtagagagttataggggcccctctcaggtagctgaa |
| | | | sense | | RNA | | tgatttaacactgctagctagagtagtcatggccataactatgatggaagttcctacggtttcataccttgattctact |
| | | | | | | | gccttgcacagctctgaaagaatacccactgaagccagatgcgaaaacgctctggcattagcatggtcattatcagtg |
| | | | | | | | acctgcttggcttctacctcaaactgcgggaagctccgctgcaaagctctgaggaatgggctgtcttcctcgatgtc |
| | | | | | | | aacgtgaactttctccattttgggtaggtaattggtctgggcttctctcatgcgccgcccat |
| 257 | VEEV | antis_3p_VEEV_ | anti- | R | DNA or | 477 | tgggccaggattctcctcgacgtcaccgcatgttagcagacttcctctgccctctccactgcctgaagccagatgcg |
| | | V2 | sense | | RNA | | aaaacgctctggcattagcatggtcattatcagtgacctgcttggcttctacctcaaactgcgggaagctccgctgca |
| | | | | | | | aagctctgaggaatgggctgtcttcctcgatgtcaacgtgaactttctccattttgggtaggtaattggtctgggcttc |
| | | | | | | | tctcatgcgccgcccattcaggttagccgtagagagttataggggcccctctcagtgaagccagatgcgaaaacgc |
| | | | | | | | tctggcattagcatggtcattatcagtgacctgcttggcttctacctcaaactgcgggaagctccgctgcaaagctct |
| | | | | | | | gaggaatgggctgtcttcctcgatgtcaacgtgaactttctccattttgggtaggtaattggtctgggcttctctcatg |
| | | | | | | | cgccgcccat |
| 258 | VEEV | sense_5p_VEEV | sense | L | DNA or | 373 | atgggcggcgcatgagagaagcccagaccaattacctacccaaaatggagaaagttcacgttgacatcgaggaa |
| | | | | | RNA | | gacagcccattcctcagagctttgcagcggagcttcccgcagtttgaggtagaagccaagcaggtcactgataatg |
| | | | | | | | accatgctaatgccagagcgttttcgcatctggcttcagtgggtattctttcagagctgtgcaaggcagtagaatcaa |
| | | | | | | | ggtatgaaaccgtaggaacttccatcatagttatggccatgactactctagctagcagtgttaaatcattcagctacct |
| | | | | | | | gagaggggcccctataactctctacggctaacctgaatggactacgacatagtctagtccgccaag |
| 259 | VEEV | sense_5p_VEEV_ | sense | L | DNA or | 477 | atgggcggcgcatgagagaagcccagaccaattacctacccaaaatggagaaagttcacgttgacatcgaggaa |
| | | V2 | | | RNA | | gacagcccattcctcagagctttgcagcggagcttcccgcagtttgaggtagaagccaagcaggtcactgataatg |
| | | | | | | | accatgctaatgccagagcgttttcgcatctggcttcactgagaggggcccctataactctctacggctaacctgaat |
| | | | | | | | gggcggcgcatgagagaagcccagaccaattacctacccaaaatggagaaagttcacgttgacatcgaggaag |
| | | | | | | | acagcccattcctcagagctttgcagcggagcttcccgcagtttgaggtagaagccaagcaggtcactgataatga |
| | | | | | | | ccatgctaatgccagagcgttttcgcatctggcttcaggcagtggagagggcagaggaagtctgctaacatgcggt |
| | | | | | | | gacgtcgaggagaatcctggccca |
| 260 | VEEV | sense_3p_VEEV | sense | R | DNA or | 144 | gaatacagcagcaattggcaagctgcttacatagaactcgcggcgattggcatgccgccttaaaatttttattttatt |
| | | | | | RNA | | ttttcttttcttttccgaatcggattttgtttttaatatttcaaaaaaaaaaaaaaaaaaaaaaaa |
| 261 | WEEV | antis_5p_WEEV | anti- | L | DNA or | 315 | tttttttttttttttgaaattttaaaaacaaaataaaaacaaaagaaaaattataaaagaatttataaagtgtgtatag |
| | | | sense | | RNA | | tgtttttaagtaaaatatgtgtagtgtatattagagacccatatgagtctcataaaagtgatatagtgttgttaatatg |
| | | | | | | | tagtgtgatatacgattaatgtgacactgcacagcattatgcactgtgcttcctcggaaatacatcgagttttaatgtc |
| | | | | | | | aagcggcgcgtggcattatgcaccacgcttcctcggaagtacatcgagttttaccgctatgtcagtggccgcgctcag |
| 262 | WEEV | antis_3p_WEEV | anti- | R | DNA or | 266 | tttggcggtgggtaggtgcgtgtctactacgtcacctatttaggtcagccgtagagggtgattgggttccctcttattg |
| | | | sense | | RNA | | ccacatgcgaaaacgctctggcattggcatggtcattgtcagtgacctgccttgcttcgatctcaaattgtggaaagc |
| | | | | | | | tccgctgtaacgacttgacatacgggctgtcagcatctaagtcaacgtgaattctttccatattggatcgattagtttg |
| | | | | | | | tagggtaggttcctctataccgtgccctat |
| 263 | WEEV | antis_3p_ | anti- | R | DNA or | 479 | tgggccaggattctcctcgacgtcaccgcatgttagcagacttcctctgccctctccactgcctgccacatgcgaaa |
| | | WEEV_V2 | sense | | RNA | | acgctctggcattggcatggtcattgtcagtgacctgccttgcttcgatctcaaattgtggaaagctccgctgtaacg |
| | | | | | | | acttgacatacgggctgtcagcatctaagtcaacgtgaattctttccatattggatcgattagtttgtagggtaggttc |
| | | | | | | | ctctataccgtgccctatttaggtcagccgtagagggtgattgggttccctcttattgccacatgcgaaaacgctctgg |
| | | | | | | | cattggcatggtcattgtcagtgacctgccttgcttcgatctcaaattgtggaaagctccgctgtaacgacttgacata |
| | | | | | | | cgggctgtcagcatctaagtcaacgtgaattctttccatattggatcgattagtttgtagggtaggttcctctataccg |
| | | | | | | | tgccctat |
| 264 | WEEV | sense_5p_WEEV | sense | L | DNA or | 266 | atagggcacggtatagaggaacctaccctacaaactaatcgatccaatatggaaagaattcacgttgacttagatgc |
| | | | | | RNA | | tgacagcccgtatgtcaagtcgttacagcggagctttccacaatttgagatcgaagcaaggcaggtcactgacaat |
| | | | | | | | gaccatgccaatgccagagcgttttcgcatgtggcaataagagggaacccaatcaccctctacggctgacctaaat |
| | | | | | | | aggtgacgtagtagacacgcacctacccaccgccaaa |
| 265 | WEEV | sense_5p_ | sense | L | DNA or | 479 | atagggcacggtatagaggaacctaccctacaaactaatcgatccaatatggaaagaattcacgttgacttagatgc |
| | | WEEV_V2 | | | RNA | | tgacagcccgtatgtcaagtcgttacagcggagctttccacaatttgagatcgaagcaaggcaggtcactgacaat |
| | | | | | | | gaccatgccaatgccagagcgttttcgcatgtggcaataagagggaacccaatcaccctctacggctgacctaaat |
| | | | | | | | agggcacggtatagaggaacctaccctacaaactaatcgatccaatatggaaagaattcacgttgacttagatgctg |
| | | | | | | | acagcccgtatgtcaagtcgttacagcggagctttccacaatttgagatcgaagcaaggcaggtcactgacaatga |
| | | | | | | | ccatgccaatgccagagcgttttcgcatgtggcaggcagtggagagggcagaggaagtctgctaacatgcggtg |
| | | | | | | | acgtcgaggagaatcctggccca |
| 266 | WEEV | sense_3p_WEEV | sense | R | DNA or | 322 | ctgagcgcggccactgacatagcggtaaaactcgatgtacttccgaggaagcgtggtgcataatgccacgcgcc |
| | | | | | RNA | | gcttgacattaaaactcgatgtatttccgaggaagcacagtgcataatgctgtgcagtgtcacattaatcgtatatcac |
| | | | | | | | actacatattaacaacactatatcacttttatgagactcatatgggtctctaatatacactacacatattttacttaaa |
| | | | | | | | aacactatacacactttataaattcttttataatttttttttgtttttattttgtttttaaaatttcaaaaaaaaaaaa |
| | | | | | | | aaaaaaaaaa |

TABLE 2
Exemplary Encrypted RNA Scaffolds

| | | L Region | | R Region | | |
|---|---|---|---|---|---|---|
| | | | SEQ | | SEQ | |
| Encrypted | | | ID | | ID | Additional |
| RNA Scaffold | Encryption | Name | NO: | Name | NO: | Information |
| ERNA-IAV-001 | antisense | antis_5p_IAV | 1 | antis_3p_IAV | 18 | |
| ERNA-IAV-002 | antisense | antis_5p_IAV | 1 | antis_3p_IAV_enhanced | 19 | |
| ERNA-IAV-003 | antisense | antis_5p_IAV_seg01 | 2 | antis_3p_IAV_seg01 | 20 | |
| ERNA-IAV-004 | antisense | antis_5p_IAV_seg02 | 3 | antis_3p_IAV_seg02 | 24 | |
| ERNA-IAV-005 | antisense | antis_5p_IAV_seg03 | 4 | antis_3p_IAV_seg03 | 28 | |
| ERNA-IAV-006 | antisense | antis_5p_IAV_seg04 | 5 | antis_3p_IAV | 18 | |
| ERNA-IAV-007 | antisense | antis_5p_IAV_seg05 | 6 | antis_3p_IAV_seg05 | 32 | |
| ERNA-IAV-008 | antisense | antis_5p_IAV_seg06 | 7 | antis_3p_IAV_seg06 | 34 | |
| ERNA-IAV-009 | antisense | antis_5p_IAV_seg07 | 8 | antis_3p_IAV_seg07 | 38 | |
| ERNA-IAV-010 | antisense | antis_5p_IAV_seg08 | 9 | antis_3p_IAV_seg08 | 42 | |
| ERNA-IAV-011 | antisense | antis_5p_IAV_seg01 | 2 | antis_3p_IAV_seg01_enhanced | 21 | |
| ERNA-IAV-012 | antisense | antis_5p_IAV_seg02 | 3 | antis_3p_IAV_seg02_enhanced | 25 | |
| ERNA-IAV-013 | antisense | antis_5p_IAV_seg03 | 4 | antis_3p_IAV_seg03_enhanced | 29 | |
| ERNA-IAV-014 | antisense | antis_5p_IAV_seg04 | 5 | antis_3p_IAV_enhanced | 19 | |
| ERNA-IAV-015 | antisense | antis_5p_IAV_seg05 | 6 | antis_3p_IAV_seg05_enhanced | 33 | |
| ERNA-IAV-016 | antisense | antis_5p_IAV_seg06 | 7 | antis_3p_IAV_seg06_enhanced | 35 | |
| ERNA-IAV-017 | antisense | antis_5p_IAV_seg07 | 8 | antis_3p_IAV_seg07_enhanced | 39 | |
| ERNA-IAV-018 | antisense | antis_5p_IAV_seg08 | 9 | antis_3p_IAV_seg08_enhanced | 43 | |
| ERNA-IAV-019 | antisense | antis_5p_IAV_seg01 | 2 | antis_3p_IAV_seg01_m4 | 22 | |
| ERNA-IAV-020 | antisense | antis_5p_IAV_seg02 | 3 | antis_3p_IAV_seg02_m4 | 26 | |
| ERNA-IAV-021 | antisense | antis_5p_IAV_seg03 | 4 | antis_3p_IAV_seg03_m4 | 30 | |
| ERNA-IAV-022 | antisense | antis_5p_IAV_seg06 | 7 | antis_3p_IAV_seg06_m4 | 36 | |
| ERNA-IAV-023 | antisense | antis_5p_IAV_seg07 | 8 | antis_3p_IAV_seg07_m4 | 40 | |
| ERNA-IAV-024 | antisense | antis_5p_IAV_seg01 | 2 | antis_3p_IAV_seg01_m4_enhanced | 23 | |
| ERNA-IAV-025 | antisense | antis_5p_IAV_seg02 | 3 | antis_3p_IAV_seg02_m4_enhanced | 27 | |
| ERNA-IAV-026 | antisense | antis_5p_IAV_seg03 | 4 | antis_3p_IAV_seg03_m4_enhanced | 31 | |
| ERNA-IAV-027 | antisense | antis_5p_IAV_seg06 | 7 | antis_3p_IAV_seg06_m4_enhanced | 37 | |
| ERNA-IAV-028 | antisense | antis_5p_IAV_seg07 | 8 | antis_3p_IAV_seg07_m4_enhanced | 41 | |
| ERNA-IBV-001 | antisense | antis_5p_IBV | 10 | antis_3p_IBV | 44 | |
| ERNA-IBV-002 | antisense | antis_5p_IBV | 10 | antis_3p_IBV_enhanced | 45 | |
| ERNA-IBV-003 | antisense | antis_5p_IBV_seg01 | 11 | antis_3p_IBV_seg01 | 46 | |
| ERNA-IBV-004 | antisense | antis_5p_IBV_seg02 | 12 | antis_3p_IBV_seg02 | 48 | |
| ERNA-IBV-005 | antisense | antis_5p_IBV_seg03 | 13 | antis_3p_IBV_seg03 | 50 | |
| ERNA-IBV-006 | antisense | antis_5p_IBV_seg05 | 14 | antis_3p_IBV_seg05 | 52 | |
| ERNA-IBV-007 | antisense | antis_5p_IBV_seg06 | 15 | antis_3p_IBV_seg06 | 54 | |
| ERNA-IBV-008 | antisense | antis_5p_IBV_seg07 | 16 | antis_3p_IBV_seg07 | 56 | |
| ERNA-IBV-009 | antisense | antis_5p_IBV_seg08 | 17 | antis_3p_IBV_seg08 | 58 | |
| ERNA-IBV-010 | antisense | antis_5p_IBV_seg01 | 11 | antis_3p_IBV_seg01_enhanced | 47 | |
| ERNA-IBV-011 | antisense | antis_5p_IBV_seg02 | 12 | antis_3p_IBV_seg02_enhanced | 49 | |
| ERNA-IBV-012 | antisense | antis_5p_IBV_seg03 | 13 | antis_3p_IBV_seg03_enhanced | 51 | |
| ERNA-IBV-013 | antisense | antis_5p_IBV_seg05 | 14 | antis_3p_IBV_seg05_enhanced | 53 | |
| ERNA-IBV-014 | antisense | antis_5p_IBV_seg06 | 15 | antis_3p_IBV_seg06_enhanced | 55 | |
| ERNA-IBV-015 | antisense | antis_5p_IBV_seg07 | 16 | antis_3p_IBV_seg07_enhanced | 57 | |
| ERNA-IBV-016 | antisense | antis_5p_IBV_seg08 | 17 | antis_3p_IBV_seg08_enhanced | 59 | |
| ERNA-SARS1-001 | sense | sense_5p_SARS1 | 60 | sense_3p_SARS1 | 129 | |
| ERNA-SARS1-002 | sense | sense_5p_SARS1_nsp1_124 | 65 | sense_3p_SARS1 | 129 | |
| ERNA-SARS1-003 | sense | sense_5p_SARS1_nsp1_164 | 67 | sense_3p_SARS1 | 129 | |
| ERNA-SARS1-004 | sense | sense_5p_SARS1_nsp1_124_164 | 66 | sense_3p_SARS1 | 129 | |
| ERNA-SARS1-501 | antisense | antis_5p_SARS1 | 137 | antis_3p_SARS1_short | 128 | |
| ERNA-SARS1-101 | sense | sense_5p_SARS1GL | 61 | sense_3p_SARS1 | 129 | |
| ERNA-SARS1-102 | sense | sense_5p_SARS1GL_nsp1_124 | 62 | sense_3p_SARS1 | 129 | |
| ERNA-SARS1-103 | sense | sense_5p_SARS1GL_nsp1_164 | 64 | sense_3p_SARS1 | 129 | |
| ERNA-SARS1-104 | sense | sense_5p_SARS1GL_nsp1_124_164 | 63 | sense_3p_SARS1 | 129 | |
| ERNA-SARS2-001 | sense | sense_5p_SARS2 | 68 | sense_3p_SARS2 | 130 | |
| ERNA-SARS2-002 | sense | sense_5p_SARS2_N250 | 109 | sense_3p_SARS2 | 130 | |
| ERNA-SARS2-003 | sense | sense_5p_SARS2_N250_HP10 | 110 | sense_3p_SARS2 | 130 | |
| ERNA-SARS2-004 | sense | sense_5p_SARS2_N250_HP30 | 111 | sense_3p_SARS2 | 130 | |
| ERNA-SARS2-005 | sense | sense_5p_SARS2_N250_HP50 | 112 | sense_3p_SARS2 | 130 | |
| ERNA-SARS2-006 | sense | sense_5p_SARS2_N250_us_ATG | 114 | sense_3p_SARS2 | 130 | |
| ERNA-SARS2-007 | sense | sense_5p_SARS2_ATG_HP15 | 104 | sense_3p_SARS2 | 130 | |
| ERNA-SARS2-008 | sense | sense_5p_SARS2_ATG_HP30 | 105 | sense_3p_SARS2 | 130 | |
| ERNA-SARS2-009 | sense | sense_5p_SARS2_ATG_HP45 | 106 | sense_3p_SARS2 | 130 | |
| ERNA-SARS2-010 | sense | sense_5p_SARS2_ATG_HP60 | 107 | sense_3p_SARS2 | 130 | |
| ERNA-SARS2-011 | sense | sense_5p_SARS2_N250_stop | 113 | sense_3p_SARS2 | 130 | |
| ERNA-SARS2-012 | sense | sense_5p_SARS2_hp_ATG2 | 119 | sense_3p_SARS2 | 130 | |
| ERNA-SARS2-013 | sense | sense_5p_SARS2_uATG_overlap | 127 | sense_3p_SARS2 | 130 | |
| ERNA-SARS2-014 | sense | sense_5p_SARS2_uATG_NDegron1 | 125 | sense_3p_SARS2 | 130 | |
| ERNA-SARS2-015 | sense | sense_5p_SARS2_uATG_NDegron2 | 126 | sense_3p_SARS2 | 130 | |
| ERNA-SARS2-016 | sense | sense_5p_SARS2_nsp1_124 | 122 | sense_3p_SARS2 | 130 | |
| ERNA-SARS2-017 | sense | sense_5p_SARS2_nsp1_164 | 124 | sense_3p_SARS2 | 130 | |
| ERNA-SARS2-018 | sense | sense_5p_SARS2_nsp1_124_164 | 123 | sense_3p_SARS2 | 130 | |
| ERNA-SARS2-019 | sense | sense_5p_SARS2_GGC_HHRz | 108 | sense_3p_SARS2 | 130 | |
| ERNA-SARS2-020 | sense | sense_5p_SARS2_PolII_ΔSA_ΔSD | 115 | sense_3p_SARS2 | 130 | |
| ERNA-SARS2-021 | sense | sense_5p_SARS2_UTRuATGv1 | 116 | sense_3p_SARS2 | 130 | |
| ERNA-SARS2-022 | sense | sense_5p_SARS2_UTRuATGv2 | 117 | sense_3p_SARS2 | 130 | |
| ERNA-SARS2-023 | sense | sense_5p_SARS2_UTRuATGv3 | 118 | sense_3p_SARS2 | 130 | |
| ERNA-SARS2-024 | sense | sense_5p_SARS2_hp_ATG3 | 120 | sense_3p_SARS2 | 130 | |
| ERNA-SARS2-025 | sense | sense_5p_SARS2_nonactivatable | 121 | sense_3p_SARS2 | 130 | non- |
| | | | | | | functional |
| ERNA-SARS2-101 | sense | sense_5p_SARS2GL | 69 | sense_3p_SARS2 | 130 | |
| ERNA-SARS2-102 | sense | sense_5p_SARS2GL_N250 | 75 | sense_3p_SARS2 | 130 | |
| ERNA-SARS2-103 | sense | sense_5p_SARS2GL_N250_HP10 | 76 | sense_3p_SARS2 | 130 | |
| ERNA-SARS2-104 | sense | sense_5p_SARS2GL_N250_HP30 | 77 | sense_3p_SARS2 | 130 | |
| ERNA-SARS2-105 | sense | sense_5p_SARS2GL_N250_HP50 | 78 | sense_3p_SARS2 | 130 | |
| ERNA-SARS2-106 | sense | sense_5p_SARS2GL_N250_us_ATG | 80 | sense_3p_SARS2 | 130 | |
| ERNA-SARS2-107 | sense | sense_5p_SARS2GL_ATG_HP15 | 70 | sense_3p_SARS2 | 130 | |
| ERNA-SARS2-108 | sense | sense_5p_SARS2GL_ATG_HP30 | 71 | sense_3p_SARS2 | 130 | |
| ERNA-SARS2-109 | sense | sense_5p_SARS2GL_ATG_HP45 | 72 | sense_3p_SARS2 | 130 | |
| ERNA-SARS2-110 | sense | sense_5p_SARS2GL_ATG_HP60 | 73 | sense_3p_SARS2 | 130 | |
| ERNA-SARS2-111 | sense | sense _5p_SARS2GL_N250_stop | 79 | sense_3p_SARS2 | 130 | |
| ERNA-SARS2-112 | sense | sense_5p_SARS2GL_hp_ATG2 | 82 | sense_3p_SARS2 | 130 | |
| ERNA-SARS2-113 | sense | sense_5p_SARS2GL_uATG_overlap | 90 | sense_3p_SARS2 | 130 | |
| ERNA-SARS2-114 | sense | sense_5p_SARS2GL_uATG_NDegron1 | 88 | sense_3p_SARS2 | 130 | |
| ERNA-SARS2-115 | sense | sense_5p_SARS2GL_uATG_NDegron2 | 89 | sense_3p_SARS2 | 130 | |
| ERNA-SARS2-116 | sense | sense_5p_SARS2GL_nsp1_124 | 85 | sense_3p_SARS2 | 130 | |
| ERNA-SARS2-117 | sense | sense_5p_SARS2GL_nsp1_164 | 87 | sense_3p_SARS2 | 130 | |
| ERNA-SARS2-118 | sense | sense_5p_SARS2GL_nsp1_124_164 | 86 | sense_3p_SARS2 | 130 | |
| ERNA-SARS2-119 | sense | sense_5p_SARS2GL_GGC_HHRz | 74 | sense_3p_SARS2 | 130 | |
| ERNA-SARS2-120 | sense | sense_5p_SARS2GL_PolII_ΔSA_ΔSD | 81 | sense_3p_SARS2 | 130 | |
| ERNA-SARS2-121 | sense | sense_5p_SARS2GL_hp_ATG3 | 83 | sense_3p_SARS2 | 130 | |
| ERNA-SARS2-122 | sense | sense_5p_SARS2GL_nonactivatable | 84 | sense_3p_SARS2 | 130 | non- |
| | | | | | | functional |
| | | | | | | control |
| ERNA-SARS2-123 | sense | sense_5p_SARS2GL_A300_locA | 91 | sense_3p_SARS2 | 130 | |
| ERNA-SARS2-124 | sense | sense_5p_SARS2GL_A300_locB | 92 | sense_3p_SARS2 | 130 | |
| ERNA-SARS2-125 | sense | sense_5p_SARS2GL_A300_locC | 93 | sense_3p_SARS2 | 130 | non- |
| | | | | | | functional |
| | | | | | | control |
| ERNA-SARS2-126 | sense | sense_5p_SARS2GL_A300_locD | 94 | sense_3p_SARS2 | 130 | non- |
| | | | | | | functional |
| | | | | | | control |
| ERNA-SARS2-127 | sense | sense_5p_SARS2GL_A30_loc01 | 95 | sense_3p_SARS2 | 130 | non- |
| | | | | | | functional |
| | | | | | | control |
| ERNA-SARS2-128 | sense | sense_5p_SARS2GL_A30_loc02 | 96 | sense_3p_SARS2 | 130 | |
| ERNA-SARS2-129 | sense | sense_5p_SARS2GL_A30_loc03 | 97 | sense_3p_SARS2 | 130 | non- |
| | | | | | | functional |
| | | | | | | control |
| ERNA-SARS2-130 | sense | sense_5p_SARS2GL_A30_loc04 | 98 | sense_3p_SARS2 | 130 | non- |
| | | | | | | functional |
| | | | | | | control |
| ERNA-SARS2-131 | sense | sense_5p_SARS2GL_A30_loc05 | 99 | sense_3p_SARS2 | 130 | non- |
| | | | | | | functional |
| | | | | | | control |
| ERNA-SARS2-132 | sense | sense_5p_SARS2GL_A30_loc06 | 100 | sense_3p_SARS2 | 130 | non- |
| | | | | | | functional |
| | | | | | | control |
| ERNA-SARS2-133 | sense | sense_5p_SARS2GL_A30_loc07 | 101 | sense_3p_SARS2 | 130 | non- |
| | | | | | | functional |
| | | | | | | control |
| ERNA-SARS2-134 | sense | sense_5p_SARS2GL_A30_loc08 | 102 | sense_3p_SARS2 | 130 | non- |
| | | | | | | functional |
| | | | | | | control |
| ERNA-SARS2-135 | sense | sense_5p_SARS2GL_A30_loc09 | 103 | sense_3p_SARS2 | 130 | non- |
| | | | | | | functional |
| | | | | | | control |
| ERNA-SARS2-136 | sense | sense_5p_SARS2GL | 69 | sense_3p_SARS2GL_Δ30_loc10 | 131 | non- |
| | | | | | | functional |
| | | | | | | control |
| ERNA-SARS2-137 | sense | sense_5p_SARS2GL | 69 | sense_3p_SARS2GL_Δ30_loc11 | 132 | non- |
| | | | | | | functional |
| | | | | | | control |
| ERNA-SARS2-138 | sense | sense_5p_SARS2GL | 69 | sense_3p_SARS2GL_Δ30_loc12 | 133 | non- |
| | | | | | | functional |
| | | | | | | control |
| ERNA-SARS2-139 | sense | sense_5p_SARS2GL | 69 | sense_3p_SARS2GL_Δ30_loc13 | 134 | non- |
| | | | | | | functional |
| | | | | | | control |
| ERNA-SARS2-140 | sense | sense_5p_SARS2GL | 69 | sense_3p_SARS2GL_Δ30_loc14 | 135 | non- |
| | | | | | | functional |
| | | | | | | control |
| ERNA-SARS2-501 | antisense | antis_5p_SARS2 | 138 | antis_3p_SARS2 | 145 | |
| ERNA-SARS2-502 | antisense | antis_5p_SARS2_invert_pA_HDVR | 144 | sense_3p_SARS2 | 130 | |
| ERNA-SARS2-503 | antisense | antis_5p_SARS2_invert_pA | 143 | antis_3p_SARS2_invert_IRES | 147 | |
| ERNA-SARS2-504 | antisense | antis_5p_SARS2_invert_pA_HDVR | 144 | antis_3p_SARS2_invert_IRES | 147 | |
| ERNA-SARS2-505 | antisense | antis_5p_SARS2_B | 142 | antis_3p_SARS2 | 145 | |
| ERNA-SARS2-506 | antisense | antis_5p_SARS2_B | 142 | antis_3p_SARS2_short | 136 | |
| ERNA-SARS2-601 | antisense | antis_5p_SARS2 | 138 | antis_3p_SARS2GL | 146 | |
| ERNA-SARS2-602 | antisense | antis_5p_SARS2GL_invert_pA_HDVR | 141 | sense_3p_SARS2 | 130 | |
| ERNA-SARS2-603 | antisense | antis_5p_SARS2GL_invert_pA | 140 | antis_3p_SARS2_invert_IRES | 147 | |
| ERNA-SARS2-604 | antisense | antis_5p_SARS2GL_invert_pA_HDVR | 141 | antis_3p_SARS2_invert_IRES | 147 | |
| ERNA-SARS2-605 | antisense | antis_5p_SARS2GL_B | 139 | antis_3p_SARS2GL | 146 | |
| ERNA-SARS2-606 | antisense | antis_5p_SARS2GL_B | 139 | antis_3p_SARS2_short | 136 | |
| ERNA-RSV-001 | antisense | antis_5p_RSV | 158 | antis_3p_RSV | 169 | |
| ERNA-RSV-002 | antisense | antis_5p_RSV_Δ56 | 168 | antis_3p_RSV | 169 | non- |
| | | | | | | functional |
| | | | | | | control |
| ERNA-RSV-003 | antisense | antis_5p_RSV_Δ115 | 167 | antis_3p_RSV | 169 | non- |
| | | | | | | functional |
| | | | | | | control |
| ERNA-RSV-004 | antisense | antis_5p_RSV | 158 | antis_3p_RSV_Δleader | 178 | non- |
| | | | | | | functional |
| | | | | | | control |
| ERNA-RSV-005 | antisense | antis_5p_RSV | 158 | antis_3p_RSV_36TrC | 170 | |
| ERNA-RSV-006 | antisense | antis_5p_RSV_ag | 164 | antis_3p_RSV_36TrC | 170 | non- |
| | | | | | | functional |
| | | | | | | control |
| ERNA-RSV-007 | antisense | antis_5p_RSV_ag | 164 | antis_3p_RSV_36TrC_ag | 175 | non- |
| | | | | | | functional |
| | | | | | | control |
| ERNA-RSV-008 | antisense | antis_5p_RSV_add_agg | 163 | antis_3p_RSV_36TrC | 170 | |
| ERNA-RSV-009 | antisense | antis_5p_RSV_add_agg | 163 | antis_3p_RSV_36TrC_ag | 175 | non- |
| | | | | | | functional |
| | | | | | | control |
| ERNA-RSV-010 | antisense | antis_5p_RSV_no_flank | 165 | antis_3p_RSV_36TrC_no_flank | 176 | |
| ERNA-RSV-011 | antisense | antis_5p_RSV_short | 166 | antis_3p_RSV_short | 177 | |
| ERNA-RSV-012 | antisense | antis_5p_RSV_short | 166 | antis_3p_RSV_36TrC | 170 | |
| ERNA-RSV-013 | antisense | antis_5p_RSV_add_021nt | 159 | antis_3p_RSV_36TrC_add_021nt | 171 | |
| ERNA-RSV-014 | antisense | antis_5p_RSV_add_051nt | 160 | antis_3p_RSV_36TrC_add_051nt | 172 | |
| ERNA-RSV-015 | antisense | antis_5p_RSV_add_101nt | 161 | antis_3p_RSV_36TrC_add_101nt | 173 | |
| ERNA-RSV-016 | antisense | antis_5p_RSV_add_234nt | 162 | antis_3p_RSV_36TrC_add_274nt | 174 | |
| ERNA-RSV-017 | antisense | antis_5p_RSV_short_add_agg | 419 | antis_3p_RSV_short | 177 | |
| ERNA-RSV-018 | antisense | antis_5p_RSV_short | 166 | antis_3p_RSV_short_36TrC | 420 | |
| ERNA-RSV-019 | antisense | antis_5p_RSV_short_add_agg | 419 | antis_3p_RSV_short_36TrC | 420 | |
| ERNA-RSV-101 | sense | sense_5p_RSV | 148 | sense_3p_RSV | 154 | |
| ERNA-RSV-102 | sense | sense_5p_RSV | 148 | sense_3p_RSV_A56 | 157 | non- |
| | | | | | | functional |
| | | | | | | control |
| ERNA-RSV-103 | sense | sense_5p_RSV | 148 | sense_3p_RSV_A115 | 156 | non- |
| | | | | | | functional |
| | | | | | | control |
| ERNA-RSV-104 | sense | sense_5p_RSV_Δleader | 153 | sense_3p_RSV | 154 | non- |
| | | | | | | functional |
| | | | | | | control |
| ERNA-RSV-105 | sense | sense_5p_RSV_short | 149 | sense_3p_RSV_short | 155 | |
| ERNA-RSV-106 | sense | sense_5p_RSV_short_add_agg | 151 | sense_3p_RSV_short | 155 | |
| ERNA-RSV-107 | sense | sense_5p_RSV_short_3610 | 150 | sense_3p_RSV_short | 155 | |
| ERNA-RSV-108 | sense | sense_5p_RSV_short_agg_3610 | 152 | sense_3p_RSV_short | 155 | |
| ERNA-HPIV1-001 | antisense | antis_5p_HPIV1_FR2011 | 181 | antis_3p_HPIV1_FR2011 | 184 | |
| ERNA-HPIV1-002 | antisense | antis_5p_HPIV1_FR2011_add_agg | 182 | antis_3p_HPIV1_FR2011 | 184 | |
| ERNA-HPIV1-003 | antisense | antis_5p_HPIV1_FR2011_t28g | 183 | antis_3p_HPIV1_FR2011 | 184 | |
| ERNA-HPIV1-501 | sense | sense_5p_HPIV1_FR2011 | 180 | sense_3p_HPIV1_FR2011 | 179 | |
| ERNA-HPIV3-001 | antisense | antis_5p_HPIV3_Wash | 187 | antis_3p_HPIV3_Wash | 190 | |
| ERNA-HPIV3-002 | antisense | antis_5p_HPIV3_Wash_add_agg | 188 | antis_3p_HPIV3_Wash | 190 | |
| ERNA-HPIV3-003 | antisense | antis_5p_HPIV3_Wash_t28g | 189 | antis_3p_HPIV3_Wash | 190 | |
| ERNA-HPIV3-501 | sense | sense_5p_HPIV3_Wash | 186 | sense_3p_HPIV3_Wash | 185 | |
| ERNA-HMPV-001 | antisense | antis_5p_HMPV_NL | 196 | antis_3p_HMPV_NL | 201 | |
| ERNA-HMPV-002 | antisense | antis_5p_HMPV_NL_s2 | 198 | antis_3p_HMPV_NL | 201 | |
| ERNA-HMPV-003 | antisense | antis_5p_HMPV_NL_add_agg | 197 | antis_3p_HMPV_NL | 201 | |
| ERNA-HMPV-004 | antisense | antis_5p_HMPV_NL_s2_add_agg | 199 | antis_3p_HMPV_NL | 201 | |
| ERNA-HMPV-005 | antisense | antis_5p_AMPV | 195 | antis_3p_AMPV | 200 | |
| ERNA-HMPV-501 | sense | sense_5p_HMPV_NL | 194 | sense_3p_HMPV_NL | 192 | |
| ERNA-HMPV-502 | sense | sense_5p_AMPV | 193 | sense_3p_AMPV | 191 | |
| ERNA-NIV-001 | antisense | antis_5p_NiV_UMMC1 | 204 | antis_3p_NiV_UMMC1 | 206 | |
| ERNA-NIV-002 | antisense | antis_5p_NiV_UMMC1_add_agg | 205 | antis_3p_NiV_UMMC1 | 206 | |
| ERNA-NIV-501 | sense | sense_5p_NiV_UMMC1 | 203 | sense_3p_NiV_UMMC1 | 202 | |
| ERNA-HeV-001 | antisense | antis_5p_HeV_AUS | 209 | antis_3p_HeV_AUS | 211 | |
| ERNA-HeV-002 | antisense | antis_5p_HeV_AUS_add_agg | 210 | antis_3p_HeV_AUS | 211 | |
| ERNA-HeV-501 | sense | sense_5p_HeV_AUS | 207 | sense_3p_HeV_AUS | 208 | |
| ERNA-HBV-001 | antisense | antis_5p_HBV_01 | 222 | antis_3p_HBV_01 | 225 | |
| ERNA-HBV-002 | antisense | antis_5p_HBV_02 | 223 | antis_3p_HBV_01 | 225 | |
| ERNA-HBV-003 | antisense | antis_5p_HBV_50 | 224 | antis_3p_HBV_50 | 226 | |
| ERNA-HBV-004 | antisense | antis_5p_HBV_50 | 224 | antis_3p_HBV_51 | 221 | |
| ERNA-HBV-101 | sense | sense_5p_HBV_21 | 212 | sense_3p_HBV_41 | 219 | |
| ERNA-HBV-102 | sense | sense_5p_HBV_41 | 214 | sense_3p_HBV_41 | 219 | |
| ERNA-HBV-103 | sense | sense_5p_HBV_31 | 213 | sense_3p_HBV_21 | 217 | |
| ERNA-HBV-104 | sense | sense_5p_HBV_31 | 213 | sense_3p_HBV_31 | 218 | |
| ERNA-HBV-105 | sense | sense_5p_HBV_60 | 215 | sense_3p_HBV_60 | 220 | |
| ERNA-HBV-106 | sense | sense_5p_HBV_61 | 216 | sense_3p_HBV_60 | 220 | |
| ERNA-ZEBOV-001 | antisense | antis_5p_ZEBOV | 227 | antis_3p_ZEBOV | 231 | |
| ERNA-ZEBOV-002 | antisense | antis_5p_ZEBOV_Δ3 | 229 | antis_3p_ZEBOV | 231 | |
| ERNA-ZEBOV-003 | antisense | antis_5p_ZEBOV_Δ3_ag | 230 | antis_3p_ZEBOV | 231 | |
| ERNA-ZEBOV-004 | antisense | antis_5p_ZEBOV_add_agg | 228 | antis_3p_ZEBOV | 231 | |
| ERNA-SEBOV-001 | antisense | antis_5p_SEBOV | 232 | antis_3p_SEBOV | 236 | |
| ERNA-SEBOV-002 | antisense | antis_5p_SEBOV_Δ3 | 234 | antis_3p_SEBOV | 236 | |
| ERNA-SEBOV-003 | antisense | antis_5p_SEBOV_Δ3_ag | 235 | antis_3p_SEBOV | 236 | |
| ERNA-SEBOV-004 | antisense | antis_5p_SEBOV_add_agg | 233 | antis_3p_SEBOV | 236 | |
| ERNA-MARV-001 | antisense | antis_5p_MARV | 237 | antis_3p_MARV | 240 | |
| ERNA-MARV-002 | antisense | antis_5p_MARV_g1a | 239 | antis_3p_MARV | 240 | |
| ERNA-MARV-003 | antisense | antis_5p_MARV_add_a | 238 | antis_3p_MARV | 240 | |
| ERNA-RV-001 | antisense | antis_5p_RV | 241 | antis_3p_RV | 242 | |
| ERNA-EVD68-001 | antisense | antis_5p_EVD68 | 243 | antis_3p_EVD68 | 244 | |
| ERNA-NDV-001 | antisense | antis_5p_NDV | 245 | antis_3p_NDV | 248 | |
| ERNA-NDV-002 | antisense | antis_5p_NDV_Δ3_add_agg | 246 | antis_3p_NDV | 248 | |
| ERNA-NDV-003 | antisense | antis_5p_NDV_ag | 247 | antis_3p_NDV | 248 | |
| ERNA-EEEV-001 | antisense | antis_5p_EEEV | 249 | antis_3p_EEEV | 250 | |
| ERNA-EEEV-002 | antisense | antis_5p_EEEV | 249 | antis_3p_EEEV_V2 | 251 | |
| ERNA-EEEV-501 | sense | sense_5p_EEEV | 252 | sense_3p_EEEV | 254 | |
| ERNA-EEEV-502 | sense | sense_5p_EEEV_V2 | 253 | sense_3p_EEEV | 254 | |
| ERNA-VEEV-001 | antisense | antis_5p_VEEV | 255 | antis_3p_VEEV | 256 | |
| ERNA-VEEV-002 | antisense | antis_5p_VEEV | 255 | antis_3p_VEEV_V2 | 257 | |
| ERNA-VEEV-501 | sense | sense_5p_VEEV | 258 | sense_3p_VEEV | 260 | |
| ERNA-VEEV-502 | sense | sense 5p_VEEV_V2 | 259 | sense_3p_VEEV | 260 | |
| ERNA-WEEV-001 | antisense | antis_5p_WEEV | 261 | antis_3p_WEEV | 262 | |
| ERNA-WEEV-002 | antisense | antis_5p_WEEV | 261 | antis_3p_WEEV_V2 | 263 | |
| ERNA-WEEV-501 | sense | sense_5p_WEEV | 264 | sense_3p_WEEV | 266 | |
| ERNA-WEEV-502 | sense | sense_5p_WEEV_V2 | 265 | sense_3p_WEEV | 266 | |

TABLE 3
Coding Sequences and Reverse Complements of Coding Sequences for Exemplary Encrypted RNAs

| | | | CDS or | Sequence | | |
|---|---|---|---|---|---|---|
| SEQ ID NO: | Name | Description | rcCDS | Type | Length | Sequence |
| 267 | CDS_RSV_N | Respiratory Syncytial | CDS | DNA or | 1176 | atggccctgagcaaagtgaagctgaacgacaccctgaacaaggatcagctgctgagcagcag |
| | | Virus (RSV) | | RNA | | caagtacaccatccagcggagcaccggcgacagcatcgacacccccaactacgacgtgcag |
| | | Nucleoprotein (N) | | | | aagcacatcaacaagctgtgcggcatgctgctgatcaccgaggacgccaaccacaagttcac |
| | | | | | | cggcctgatcggcatgctgtacgccatgagcagactgggacgcgaggacaccatcaagatcc |
| | | | | | | tgcgggacgccggctaccacgtgaaggccaatggcgtggacgtgaccacccaccggcagg |
| | | | | | | acatcaacggcaaagaaatgaagttcgaggtgctgaccctggccagcctgaccaccgagatc |
| | | | | | | cagatcaacatcgagatcgagagccggaagtcctacaagaaaatgctgaaagaaatgggcga |
| | | | | | | ggtggcccccgagtaccggcacgatagccccgactgcggcatgatcatcctgtgtatcgctgc |
| | | | | | | cctggtgatcacaaagctggccgctggcgacagatccggactgaccgctgtgatcagacggg |
| | | | | | | ccaacaacgtgctgaagaacgagatgaagcggtacaagggcctgctgcccaaggatatcgcc |
| | | | | | | aacagcttctacgaggtgttcgagaagcacccccacttcatcgacgtgttcgtgcacttcggaat |
| | | | | | | cgcccagagcagcaccagaggcggcagccgggtggaaggcatcttcgccggcctgttcatg |
| | | | | | | aacgcctacggcgctggccaggtgatgctgagatggggcgtgctggccaagagcgtgaaga |
| | | | | | | acatcatgctgggccacgccagcgtgcaggccgagatggaacaggtggtggaagtgtacga |
| | | | | | | gtacgcccagaagctgggcggcgaggccggcttctaccacatcctgaacaaccccaaggcc |
| | | | | | | agcctgctgtccctgacccagttcccccacttctccagcgtggtgctgggcaatgccgccggac |
| | | | | | | tgggcatcatgggcgagtacagaggcaccccccggaaccaggacctgtacgacgccgccaa |
| | | | | | | ggcctacgccgagcagctgaaagaaaacggcgtgatcaactacagcgtgctggatctgaccg |
| | | | | | | ccgaggaactggaagccatcaagcaccagctgaaccccaaggacaacgacgtggaactgtg |
| | | | | | | a |
| 268 | CDS_RSV_P | Respiratory Syncytial | CDS | DNA or | 381 | atggaaaagttcgcccccgagttccacggcgaggacgccaacaaccgggccaccaagtttct |
| | | Virus (RSV) | | RNA | | ggaatccatcaagggcaagttcaccagccccaaggaccccaagaagaaggacagcatcatc |
| | | Phosphoprotein (P) | | | | agcgtgaacagcatcgacatcgaagtgaccaaagagagccccatcaccagcaacagcacca |
| | | | | | | tcatcaaccccaccaacgagacagacgacaccgccggcaacaagcccaactaccagcggaa |
| | | | | | | gcccctggtgtccttcaaagaggaccccacccccagcgacaaccccttcagcaagctgtacaa |
| | | | | | | agagacaatcgagacattcgacaacaacgaggaagagagcagctacagctacgaggaaatc |
| | | | | | | aacgaccagtga |
| 269 | CDS_SARS1_N | Sudden Acute | CDS | DNA or | 1269 | atgtctgataatggaccccaatcaaaccaacgtagtgccccccgcattacatttggtggacccac |
| | | Respiratory Syndrome | | RNA | | agattcaactgacaataaccagaatggaggacgcaatggggcaaggccaaaacagcgccga |
| | | Coronavirus 1 (SARS- | | | | ccccaaggtttacccaataatactgcgtcttggttcacagctctcactcagcatggcaaggagga |
| | | CoV-1) Nucleocapsid | | | | acttagattccctcgaggccagggcgttccaatcaacaccaatagtggtccagatgaccaaatt |
| | | protein (N) | | | | ggctactaccgaagagctacccgacgagttcgtggtggtgacggcaaaatgaaagagctcag |
| | | | | | | ccccagatggtacttctattacctaggaactggcccagaagcttcacttccctacggcgctaaca |
| | | | | | | aagaaggcatcgtatgggttgcaactgagggagccttgaatacacccaaagaccacattggca |
| | | | | | | cccgcaatcctaataacaatgctgccaccgtgctacaacttcctcaaggaacaacattgccaaa |
| | | | | | | aggcttctacgcagagggaagcagaggcggcagtcaagcctcttctcgctcctcatcacgtag |
| | | | | | | tcgcggtaattcaagaaattcaactcctggcagcagtaggggaaattctcctgctcgaatggcta |
| | | | | | | gcggaggtggtgaaactgccctcgcgctattgctgctagacagattgaaccagcttgagagca |
| | | | | | | aagtttctggtaaaggccaacaacaacaaggccaaactgtcactaagaaatctgctgctgaggc |
| | | | | | | atctaaaaagcctcgccaaaaacgtactgccacaaaacagtacaacgtcactcaagcatttggg |
| | | | | | | agacgtggtccagaacaaacccaaggaaatttcggggaccaagacctaatcagacaaggaac |
| | | | | | | tgattacaaacattggccgcaaattgcacaatttgctccaagtgcctctgcattctttggaatgtca |
| | | | | | | cgcattggcatggaagtcacaccttcgggaacatggctgacttatcatggagccattaaattgga |
| | | | | | | tgacaaagatccacaattcaaagacaacgtcatactgctgaacaagcacattgacgcatacaaa |
| | | | | | | acattcccaccaacagagcctaaaaaggacaaaaagaaaaagactgatgaagctcagcctttg |
| | | | | | | ccgcagagacaaaagaagcagcccactgtgactcttcttcctgcggctgacatggatgatttctc |
| | | | | | | cagacaacttcaaaattccatgagtggagcttctgctgattcaactcaggcataa |
| 270 | CDS_SARS1_NSP3A | Sudden Acute | CDS | DNA or | 564 | atggcaccaattaaaggtgtaacctttggagaagatactgtttgggaagttcaaggttacaagaa |
| | | Respiratory Syndrome | | RNA | | tgtgagaatcacatttgagcttgatgaacgtgttgacaaagtgcttaatgaaaagtgctctgtctac |
| | | Coronavirus 1 (SARS- | | | | actgttgaatccggtaccgaagttactgagtttgcatgtgttgtagcagaggctgttgtgaagactt |
| | | CoV-1) Nonstructural | | | | tacaaccagtttctgatctccttaccaacatgggtattgatcttgatgagtggagtgtagctacattc |
| | | protein 3A domain | | | | tacttatttgatgatgctggtgaagaaaacttttcatcacgtatgtattgttccttttaccctccaga |
| | | protein (NSP3A) | | | | tgaggaagaagaggacgatgcagagtgtgaggaagaagaaattgatgaaacctgtgaacatga |
| | | | | | | gtacggtacagaggatgattatcaaggtctccctctggaatttggtgcctcagctgaaacagttc |
| | | | | | | gagttgaggaagaagaagaggaagactggctggatgatactactgagcaatcagagattgag |
| | | | | | | ccagaaccagaacctacacctgaagaaccagttaattaa |
| 271 | CDS_SARS2_N | Sudden Acute | CDS | DNA or | 1260 | atgtctgataatggaccccaaaatcagcgaaatgcaccccgcattacgtttggtggaccctcag |
| | | Respiratory Syndrome | | RNA | | attcaactggcagtaaccagaatggagaacgcagtggggcgcgatcaaaacaacgtcggccc |
| | | Coronavirus 2 (SARS- | | | | caaggtttacccaataatactgcgtcttggttcaccgctctcactcaacatggcaaggaagacctt |
| | | CoV-1) Nucleocapsid | | | | aaattccctcgaggacaaggcgttccaattaacaccaatagcagtccagatgaccaaattggct |
| | | protein (N) | | | | actaccggagagctaccagacgaattcgtggtggtgacggtaaaatgaaagatctcagtccaa |
| | | | | | | gatggtatttctactacctaggaactgggccagaagctggacttccctatggtgctaacaaagac |
| | | | | | | ggcatcatatgggttgcaactgagggagccttgaatacaccaaaagatcacattggcacccgc |
| | | | | | | aatcctgctaacaatgctgcaatcgtgctacaacttcctcaaggaacaacattgccaaaaggctt |
| | | | | | | ctacgcagaagggagcagaggcggcagtcaagcctcttctcgttcctcatcacgtagtcgcaa |
| | | | | | | cagttcaagaaattcaactccaggcagcagtaggggaacttctcctgctagaatggctggcaat |
| | | | | | | ggcggtgatgctgctcttgctttgctgctgcttgacagattgaaccagcttgagagcaaaatgtct |
| | | | | | | ggtaaaggccaacaacaacaaggccaaactgtcactaagaaatctgctgctgaggcttctaag |
| | | | | | | aagcctcggcaaaaacgtactgccactaaagcatacaatgtaacacaagctttcggcagacgt |
| | | | | | | ggtccagaacaaacccaaggaaattttggggaccaggaactaatcagacaaggaactgattac |
| | | | | | | aaacattggccgcaaattgcacaatttgcccccagcgcttcagcgttcttcggaatgtcgcgcat |
| | | | | | | tggcatggaagtcacaccttcgggaacgtggttgacctacacaggtgccatcaaattggatgac |
| | | | | | | aaagatccaaatttcaaagatcaagtcattttgctgaataagcatattgacgcatacaaaacattcc |
| | | | | | | caccaacagagcctaaaaaggacaaaaagaagaaggctgatgaaactcaagccttaccgca |
| | | | | | | gagacagaagaaacagcaaactgtgactcttcttcctgctgcagatttggatgatttctccaaaca |
| | | | | | | attgcaacaatccatgagcagtgctgactcaactcaggcctaa |
| 272 | CDS_SARS2_NSP3A | Sudden Acute | CDS | DNA or | 624 | atggcaccaacaaaggttacttttggtgatgacactgtgatagaagtgcaaggttacaagagtgt |
| | | Respiratory Syndrome | | RNA | | gaatatcacttttgaacttgatgaaaggattgataaagtacttaatgagaagtgctctgcctataca |
| | | Coronavirus 2 (SARS- | | | | gttgaactcggtacagaagtaaatgagttcgcctgtgttgtggcagatgctgtcataaaaactttg |
| | | CoV-1) Nonstructural | | | | caaccagtatctgaattacttacaccactgggcattgatttagatgagtggagtatggctacatact |
| | | protein 3A domain | | | | acttatttgatgagtctggtgagtttaaattggcttcacatatgtattgttctttctaccctccagat |
| | | protein (NSP3A) | | | | gaggatgaagaagaaggtgattgtgaagaagaagagtttgagccatcaactcaatatgagtatggt |
| | | | | | | actgaagatgattaccaaggtaaacctttggaatttggtgccacttctgctgctctgcaacctgaa |
| | | | | | | gaggagcaagaagaagattggttagatgatgatagtcaacaaactgttggtcaacaagacggc |
| | | | | | | agtgaggacaatcagacaactactattcaaacaattgttgaggttcaacctcaattagagatgga |
| | | | | | | acttacaccagttgttcagactattgaataa |
| 273 | CDS_GDura | GDura | CDS | DNA or | 558 | atgggagtcaaagttctgtttgccctgatctgcatcgctgtggccgaggccaagcccaccgaga |
| | | | | RNA | | acaacgaagacttcaacatcgtggccgtggccagcaacttcgcgaccacggatctcgatgctg |
| | | | | | | accgcgggaagttgcccggcaagaagctgccgctggaggtgctcaaagagttggaagccaa |
| | | | | | | tgcccggaaagctggctgcaccaggggctgtctgatctgcctgtcccacatcaagtgcacgcc |
| | | | | | | caagatgaagaagttcatcccaggacgctgccacacctacgaaggcgacaaagagtccgcac |
| | | | | | | agggcggcataggcgaggcgatcgtcgacattcctgagattcctgggttcaaggacttggagc |
| | | | | | | ccttggagcagttcatcgcacaggtcgatctgtgtgtggactgcacaactggctgcctcaaagg |
| | | | | | | gcttgccaacgtgcagtgttctgacctgctcaagaagtggctgccgcaacgctgtgcgaccttt |
| | | | | | | gccagcaagatccagggccaggtggacaagatcaagggggccggtggtgactaa |
| 274 | CDS_EGFP | EGFP | CDS | DNA or | 722 | atggtgagcaagggcgaggagctgttcaccggggtggtgcccatcctggtcgagctggacgg |
| | | | | RNA | | cgacgtaaacggccacaagttcagcgtgtccggcgagggcgagggcgatgccacctacggc |
| | | | | | | aagctgaccctgaagttcatctgcaccaccggcaagctgcccgtgccctggcccaccctcgtg |
| | | | | | | accaccctgacctacggcgtgcagtgcttcagccgctaccccgaccacatgaagcagcacga |
| | | | | | | cttcttcaagtccgccatgcccgaaggctacgtccaggagcgcaccatcttcttcaaggacgac |
| | | | | | | ggcaactacaagacccgcgccgaggtgaagttcgagggcgacaccctggtgaaccgcatcg |
| | | | | | | agctgaagggcatcgacttcaaggaggacggcaacatcctggggcacaagctggagtacaa |
| | | | | | | ctacaacagccacaacgtctatatcatggccgacaagcagaagaacggcatcaaggtgaactt |
| | | | | | | caagatccgccacaacatcgaggacggcagcgtgcagctcgccgaccactaccagcagaac |
| | | | | | | acccccatcggcgacggccccgtgctgctgcccgacaaccactacctgagcacccagtccgc |
| | | | | | | cctgagcaaagaccccaacgagaagcgcgatcacatggtcctgctggagttcgtgaccgccg |
| | | | | | | ccgggatcactctcggcatggacgagctgtacaagtgtaa |
| 275 | CDS_Rluc8 | Rluc8 | CDS | DNA or | 930 | atgaaggtgtacgaccccgagcagaggaagaggatgatcaccggcccccagtggtgggcca |
| | | | | RNA | | ggtgcaagcagatgaacgtgctggacagcttcatcaactactacgacagcgagaagcacgcc |
| | | | | | | gagaacgccgtgatcttcctgcacggcaacgccactagcagctacctgtggaggcacgtggt |
| | | | | | | gccccacatcgagcccgtggccaggtgcatcatccccgatctgatcggcatgggcaagagcg |
| | | | | | | gcaagagcggcaacggcagctacaggctgctggaccactacaagtacctgaccgcctggttc |
| | | | | | | gagcttctgaacctgcccaagaagatcatcttcgtgggccacgactggggcgccgccctggcc |
| | | | | | | ttccactacgcctacgagcaccaggacaggatcaaggccatcgtgcacatggagagcgtggt |
| | | | | | | ggacgtgatcgagagctgggacgagtggccagacatcgaggaggacatcgccctgatcaag |
| | | | | | | agcgaggagggcgagaagatggtgctggagaacaacttcttcgtggagaccgtgctgcccag |
| | | | | | | caagatcatgagaaagctggagcccgaggagttcgccgcctacctggagcccttcaaggaga |
| | | | | | | agggcgaggtgagaagacccaccctgagctggcccagagagatccccctggtgaagggcg |
| | | | | | | gcaagcccgacgtggtgcagatcgtgagaaactacaacgcctacctgagagccagcgacga |
| | | | | | | cctgcccaagctgttcatcgagggcgaccccggcttcttcagcaacgccatcgtggagggcgc |
| | | | | | | caagaagttccccaacaccgagttcgtgaaggtgaagggcctgcacttcctccaggaggacg |
| | | | | | | cccccgacgagatgggcaagtacatcaagagcttcgtggagagagtgctgaagaacgagca |
| | | | | | | gtaa |
| 276 | CDS_ | mNeonGreen | CDS | DNA or | 711 | atggtaagcaagggcgaggaggacaatatggcttcactcccggctacacatgaattgcatatat |
| | mNeonGreen | | | RNA | | ttggatcaataaacggggtggatttcgatatggtcggccaaggcaccggcaatcctaacgacg |
| | | | | | | gatacgaagaattgaatcttaagagtactaagggtgacttgcaattctccccgtggatattggttc |
| | | | | | | cccatatcggctacggattccatcaatacctcccttatcccgacggcatgagtccctttcaggcc |
| | | | | | | gctatggtggacggatcaggttatcaagtgcatcgaaccatgcagtttgaggatggtgcgtctct |
| | | | | | | cactgttaattataggtacacatacgaaggctcccatatcaaaggtgaggcgcaagtaaaaggc |
| | | | | | | acagggtttccggccgatggtccagttatgaccaatagccttacagccgcggactggtgtcggt |
| | | | | | | ctaaaaaaacctatccaaatgacaaaacaatcattagcacattcaagtggagctacactacgggt |
| | | | | | | aacggcaagcgatatcggagcaccgccaggacaacttataccttcgcaaagcctatggcagc |
| | | | | | | caactaccttaagaatcagcccatgtacgttttccgcaagaccgagcttaagcatagcaaaaca |
| | | | | | | gagcttaacttcaaggaatggcagaaagcgttcaccgatgtaatgggaatggacgagctgtac |
| | | | | | | aaataa |
| 277 | CDS_LacZ | LacZ | CDS | DNA or | 3075 | atgaccatgattacggattcactggccgtcgttttacaacgtcgtgactgggaaaaccctggcgt |
| | | | | RNA | | tacccaacttaatcgccttgcagcacatccccctttcgccagctggcgtaatagcgaagaggcc |
| | | | | | | cgcaccgatcgcccttcccaacagttgcgcagcctgaatggcgaatggcgctttgcctggtttc |
| | | | | | | cggcaccagaagcggtgccggaaagctggctggagtgcgatcttcctgaggccgatactgtc |
| | | | | | | gtcgtcccctcaaactggcagatgcacggttacgatgcgcccatctacaccaacgtgacctatc |
| | | | | | | ccattacggtcaatccgccgtttgttcccacggagaatccgacgggttgttactcgctcacattta |
| | | | | | | atgttgatgaaagctggctacaggaaggccagacgcgaattatttttgatggcgttaactcggcg |
| | | | | | | tttcatctgtggtgcaacgggcgctgggtcggttacggccaggacagtcgtttgccgtctgaattt |
| | | | | | | gacctgagcgcatttttacgcgccggagaaaaccgcctcgcggtgatggtgctgcgctggagt |
| | | | | | | gacggcagttatctggaagatcaggatatgtggcggatgagcggcattttccgtgacgtctcgtt |
| | | | | | | gctgcataaaccgactacacaaatcagcgatttccatgttgccactcgctttaatgatgatttcagc |
| | | | | | | cgcgctgtactggaggctgaagttcagatgtgcggcgagttgcgtgactacctacgggtaaca |
| | | | | | | gtttctttatggcagggtgaaacgcaggtcgccagcggcaccgcgcctttcggcggtgaaatta |
| | | | | | | tcgatgagcgtggtggttatgccgatcgcgtcacactacgtctgaacgtcgaaaacccgaaact |
| | | | | | | gtggagcgccgaaatcccgaatctctatcgtgcggtggttgaactgcacaccgccgacggcac |
| | | | | | | gctgattgaagcagaagcctgcgatgtcggtttccgcgaggtgcggattgaaaatggtctgctg |
| | | | | | | ctgctgaacggcaagccgttgctgattcgaggcgttaaccgtcacgagcatcatcctctgcatg |
| | | | | | | gtcaggtcatggatgagcagacgatggtgcaggatatcctgctgatgaagcagaacaactttaa |
| | | | | | | cgccgtgcgctgttcgcattatccgaaccatccgctgtggtacacgctgtgcgaccgctacggc |
| | | | | | | ctgtatgtggtggatgaagccaatattgaaacccacggcatggtgccaatgaatcgtctgaccg |
| | | | | | | atgatccgcgctggctaccggcgatgagcgaacgcgtaacgcgaatggtgcagcgcgatcgt |
| | | | | | | aatcacccgagtgtgatcatctggtcgctggggaatgaatcaggccacggcgctaatcacgac |
| | | | | | | gcgctgtatcgctggatcaaatctgtcgatccttcccgcccggtgcagtatgaaggcggcgga |
| | | | | | | gccgacaccacggccaccgatattatttgcccgatgtacgcgcgcgtggatgaagaccagcc |
| | | | | | | cttcccggctgtgccgaaatggtccatcaaaaaatggctttcgctacctggagagacgcgcccg |
| | | | | | | ctgatcctttgcgaatacgcccacgcgatgggtaacagtcttggcggtttcgctaaatactggca |
| | | | | | | ggcgtttcgtcagtatccccgtttacagggcggcttcgtctgggactgggtggatcagtcgctga |
| | | | | | | ttaaatatgatgaaaacggcaacccgtggtcggcttacggcggtgattttggcgatacgccgaa |
| | | | | | | cgatcgccagttctgtatgaacggtctggtctttgccgaccgcacgccgcatccagcgctgacg |
| | | | | | | gaagcaaaacaccagcagcagtttttccagttccgtttatccgggcaaaccatcgaagtgacca |
| | | | | | | gcgaatacctgttccgtcatagcgataacgagctcctgcactggatggtggcgctggatggtaa |
| | | | | | | gccgctggcaagcggtgaagtgcctctggatgtcgctccacaaggtaaacagttgattgaactg |
| | | | | | | cctgaactaccgcagccggagagcgccgggcaactctggctcacagtacgcgtagtgcaacc |
| | | | | | | gaacgcgaccgcatggtcagaagccgggcacatcagcgcctggcagcagtggcgtctggcg |
| | | | | | | gaaaacctcagtgtgacgctccccgccgcgtcccacgccatcccgcatctgaccaccagcga |
| | | | | | | aatggatttttgcatcgagctgggtaataagcgttggcaatttaaccgccagtcaggctttctttca |
| | | | | | | cagatgtggattggcgataaaaaacaactgctgacgccgctgcgcgatcagttcacccgtgca |
| | | | | | | ccgctggataacgacattggcgtaagtgaagcgacccgcattgaccctaacgcctgggtcgaa |
| | | | | | | cgctggaaggcggcgggccattaccaggccgaagcagcgttgttgcagtgcacggcagata |
| | | | | | | cacttgctgatgcggtgctgattacgaccgctcacgcgtggcagcatcaggggaaaaccttattt |
| | | | | | | atcagccggaaaacctaccggattgatggtagtggtcaaatggcgattaccgttgatgttgaagt |
| | | | | | | ggcgagcgatacaccgcatccggcgcggattggcctgaactgccagctggcgcaggtagca |
| | | | | | | gagcgggtaaactggctcggattagggccgcaagaaaactatcccgaccgccttactgccgc |
| | | | | | | ctgttttgaccgctgggatctgccattgtcagacatgtataccccgtacgtcttcccgagcgaaaa |
| | | | | | | cggtctgcgctgcgggacgcgcgaattgaattatggcccacaccagtggcgcggcgacttcc |
| | | | | | | agttcaacatcagccgctacagtcaacagcaactgatggaaaccagccatcgccatctgctgc |
| | | | | | | acgcggaagaaggcacatggctgaatatcgacggtttccatatggggattggtggcgacgact |
| | | | | | | cctggagcccgtcagtatcggcggaattccagctgagcgccggtcgctaccattaccagttggt |
| | | | | | | ctggtgtcaaaaataa |
| 278 | CDS_human_ | Human interferon-beta | CDS | DNA or | 564 | atgaccaacaagtgtctcctccaaattgctctcctgttgtgcttctccactacagctctttccatgag |
| | IFN_beta_1_ | precursor (Homo | | RNA | | ctacaacttgcttggattcctacaaagaagcagcaattttcagtgtcagaagctcctgtggcaatt |
| | precursor | sapiens) | | | | gaatgggaggcttgaatactgcctcaaggacaggatgaactttgacatccctgaggagattaag |
| | | | | | | cagctgcagcagttccagaaggaggacgccgcattgaccatctatgagatgctccagaacatc |
| | | | | | | tttgctattttcagacaagattcatctagcactggctggaatgagactattgttgagaacctcctgg |
| | | | | | | ctaatgtctatcatcagataaaccatctgaagacagtcctggaagaaaaactggagaaagaaga |
| | | | | | | tttcaccaggggaaaactcatgagcagtctgcacctgaaaagatattatgggaggattctgcatt |
| | | | | | | acctgaaggccaaggagtacagtcactgtgcctggaccatagtcagagtggaaatcctaagga |
| | | | | | | acttttacttcattaacagacttacaggttacctccgaaactga |
| 279 | CDS_human_ | Human interferon- | CDS | DNA or | 603 | atggctgcagcttggaccgtggtgctggtgactttggtgctaggcttggccgtggcaggccctg |
| | IFN_lambda_ | lambda precursor | | RNA | | tccccacttccaagcccaccacaactgggaagggctgccacattggcaggttcaaatctctgtc |
| | 1_precursor | (Homo sapiens) | | | | accacaggagctagcgagcttcaagaaggccagggacgccttggaagagtcactcaagctg |
| | | | | | | aaaaactggagttgcagctctcctgtcttccccgggaattgggacctgaggcttctccaggtga |
| | | | | | | gggagcgccctgtggccttggaggctgagctggccctgacgctgaaggtcctggaggccgct |
| | | | | | | gctggcccagccctggaggacgtcctagaccagccccttcacaccctgcaccacatcctctcc |
| | | | | | | cagctccaggcctgtatccagcctcagcccacagcagggcccaggccccggggccgcctcc |
| | | | | | | accactggctgcaccggctccaggaggcccccaaaaaggagtccgctggctgcctggaggc |
| | | | | | | atctgtcaccttcaacctcttccgcctcctcacgcgagacctcaaatatgtggccgatgggaacc |
| | | | | | | tgtgtctgagaacgtcaacccaccctgagtccacctga |
| 280 | CDS_human_ | Human interferon- | CDS | DNA or | 591 | atgaccggggactgcatgccagtgctggtgctgatggccgcagtgctgaccgtgactggagc |
| | IFN_lambda_ | lambda 3 precursor | | RNA | | agttcctgtcgccaggctccgcggggctctcccggatgcaaggggctgccacatagcccagtt |
| | 3_precursor | (Homo sapiens) | | | | caagtccctgtctccacaggagctgcaggcctttaagagggccaaagatgccttagaagagtc |
| | | | | | | gcttctgctgaaggactgcaagtgccgctcccgcctcttccccaggacctgggacctgaggca |
| | | | | | | gctgcaggtgagggagcgccccgtggctttggaggctgagctggccctgacgctgaaggttc |
| | | | | | | tggaggccaccgctgacactgacccagccctgggggatgtcttggaccagccccttcacacc |
| | | | | | | ctgcaccatatcctctcccagctccgggcctgtatccagcctcagcccacggcagggcccagg |
| | | | | | | acccggggccgcctccaccattggctgcaccggctccaggaggccccaaaaaaggagtccc |
| | | | | | | ctggctgcctcgaggcctctgtcaccttcaacctcttccgcctcctcacgcgagacctgaattgt |
| | | | | | | gttgccagcggggacctgtgtgtctga |
| 281 | CDS_human_ | Human interleukin-2 | CDS | DNA or | 462 | atgtacaggatgcaactcctgtcttgcattgcactaagtcttgcacttgtcacaaacagtgcacct |
| | IL2_precursor | precursor (Homo | | RNA | | acttcaagttctacaaagaaaacacagctacaactggagcatttactgctggatttacagatgattt |
| | | sapiens) | | | | tgaatggaattaataattacaagaatcccaaactcaccaggatgctcacatttaagttttacatgcc |
| | | | | | | caagaaggccacagaactgaaacatcttcagtgtctagaagaagaactcaaacctctggagga |
| | | | | | | agtgctaaatttagctcaaagcaaaaactttcacttaagacccagggacttaatcagcaatatcaa |
| | | | | | | cgtaatagttctggaactaaagggatctgaaacaacattcatgtgtgaatatgctgatgagacag |
| | | | | | | caaccattgtagaatttctgaacagatggattaccttttgtcaaagcatcatctcaacactgacttg |
| | | | | | | a |
| 282 | CDS_human_ | Human interleukin- | CDS | DNA or | 1710 | atgtgtccagcgcgcagcctcctccttgtggctaccctggtcctcctggaccacctcagtttggc |
| | IL12A_P2A_ | 12A and interleukin- | | RNA | | cagaaacctccccgtggccactccagacccaggaatgttcccatgccttcaccactcccaaaa |
| | IL12B_ | 12B precursors (bi- | | | | cctgctgagggccgtcagcaacatgctccagaaggccagacaaactctagaattttacccttgc |
| | precursors | cistronic via P2A site) | | | | acttctgaagagattgatcatgaagatatcacaaaagataaaaccagcacagtggaggcctgttt |
| | | (Homo sapiens) | | | | accattggaattaaccaagaatgagagttgcctaaattccagagagacctctttcataactaatgg |
| | | | | | | gagttgcctggcctccagaaagacctcttttatgatggccctgtgccttagtagtatttatgaagac |
| | | | | | | ttgaagatgtaccaggtggagttcaagaccatgaatgcaaagcttctgatggatcctaagaggc |
| | | | | | | agatctttctagatcaaaacatgctggcagttattgatgagctgatgcaggccctgaatttcaaca |
| | | | | | | gtgagactgtgccacaaaaatcctcccttgaagaaccggatttttataaaactaaaatcaagctct |
| | | | | | | gcatacttcttcatgctttcagaattcgggcagtgactattgatagagtgatgagctatctgaatgc |
| | | | | | | ttccggaagcggagctactaacttcagcctgctgaagcaggctggagacgtggaggagaacc |
| | | | | | | ctggacctatgtgtcaccagcagttggtcatctcttggttttccctggtttttctggcatctcccct |
| | | | | | | cgtggccatatgggaactgaagaaagatgtttatgtcgtagaattggattggtatccggatgcccct |
| | | | | | | ggagaaatggtggtcctcacctgtgacacccctgaagaagatggtatcacctggaccttggac |
| | | | | | | cagagcagtgaggtcttaggctctggcaaaaccctgaccatccaagtcaaagagtttggagat |
| | | | | | | gctggccagtacacctgtcacaaaggaggcgaggttctaagccattcgctcctgctgcttcaca |
| | | | | | | aaaaggaagatggaatttggtccactgatattttaaaggaccagaaagaacccaaaaataagac |
| | | | | | | ctttctaagatgcgaggccaagaattattctggacgtttcacctgctggtggctgacgacaatca |
| | | | | | | gtactgatttgacattcagtgtcaaaagcagcagaggctcatctgacccccaaggggtgacgtg |
| | | | | | | cggagctgctacactctctgcagagagagtcagaggggacaacaaggagtatgagtactcag |
| | | | | | | tggagtgccaggaggacagtgcctgcccagctgctgaggagagtctgcccattgaggtcatg |
| | | | | | | gtggatgccgttcacaagctcaagtatgaaaactacaccagcagcttcttcatcagggacatcat |
| | | | | | | caaacctgacccacccaagaacttgcagctgaagccattaaagaattctcggcaggtggaggt |
| | | | | | | cagctgggagtaccctgacacctggagtactccacattcctacttctccctgacattctgcgttca |
| | | | | | | ggtccagggcaagagcaagagagaaaagaaagatagagtcttcacggacaagacctcagcc |
| | | | | | | acggtcatctgccgcaaaaatgccagcattagcgtgcgggcccaggaccgctactatagctca |
| | | | | | | tcttggagcgaatgggcatctgtgccctgcagttag |
| 283 | CDS_human_ | Human interleukin-1 | CDS | DNA or | 534 | atggaaatctgcagaggcctccgcagtcacctaatcactctcctcctcttcctgttccattcagag |
| | IL1RN_isoform_ | receptor antagonist | | RNA | | acgatctgccgaccctctgggagaaaatccagcaagatgcaagccttcagaatctgggatgtta |
| | 1_precursor | precursor (Homo | | | | accagaagaccttctatctgaggaacaaccaactagttgctggatacttgcaaggaccaaatgt |
| | | sapiens) | | | | caatttagaagaaaagatagatgtggtacccattgagcctcatgctctgttcttgggaatccatgg |
| | | | | | | agggaagatgtgcctgtcctgtgtcaagtctggtgatgagaccagactccagctggaggcagtt |
| | | | | | | aacatcactgacctgagcgagaacagaaagcaggacaagcgcttcgccttcatccgctcaga |
| | | | | | | cagtggccccaccaccagttttgagtctgccgcctgccccggttggttcctctgcacagcgatg |
| | | | | | | gaagctgaccagcccgtcagcctcaccaatatgcctgacgaaggcgtcatggtcaccaaattct |
| | | | | | | acttccaggaggacgagtag |
| 284 | CDS_human_ | Human tumor necrosis | CDS | DNA or | 702 | atgagcactgaaagcatgatccgggacgtggagctggccgaggaggcgctccccaagaaga |
| | TNF_precursor | factor precursor | | RNA | | caggggggccccagggctccaggcggtgcttgttcctcagcctcttctccttcctgatcgtggc |
| | | (Homo sapiens) | | | | aggcgccaccacgcttttctgcctgctgcactttggagtgatcggcccccagagggaagagttc |
| | | | | | | cccagggacctctctctaatcagccctctggcccaggcagtcagatcatcttctcgaaccccga |
| | | | | | | gtgacaagcctgtagcccatgttgtagcaaaccctcaagctgaggggcagctccagtggctga |
| | | | | | | accgccgggccaatgccctcctggccaatggcgtggagctgagagataaccagctggtggtg |
| | | | | | | ccatcagagggcctgtacctcatctactcccaggtcctcttcaagggccaaggctgcccctcca |
| | | | | | | cccatgtgctcctcacccacaccatcagccgcatcgccgtctcctaccagaccaaggtcaacct |
| | | | | | | cctctctgccatcaagagcccctgccagagggagaccccagagggggctgaggccaagccc |
| | | | | | | tggtatgagcccatctatctgggaggggtcttccagctggagaagggtgaccgactcagcgct |
| | | | | | | gagatcaatcggcccgactatctcgactttgccgagtctgggcaggtctactttgggatcattgc |
| | | | | | | cctgtga |
| 285 | CDS_human_ | Human activated | CDS | DNA or | 873 | atgattgagacagacagtggtgttgatgatgacatggcgtgtcataaaataccagtggaggccg |
| | revCaspase3 | Caspase 3 (Homo | | RNA | | acttcttgtatgcatactccacagcacctggttattattcttggcgaaattcaaaggatggctcctg |
| | | sapiens) | | | | gttcatccagtcgctttgtgccatgctgaaacagtatgccgacaagcttgaatttatgcacattctt |
| | | | | | | acccgggttaaccgaaaggtggcaacagaatttgagtccttttcctttgacgctacttttcatgcaa |
| | | | | | | agaaacagattccatgtattgtttccatgctcacaaaagaactctatttttatcacgatgaagttgat |
| | | | | | | gggggatcccccatggagaacactgaaaactcagtggattcaaaatccattaaaaatttggaac |
| | | | | | | caaagatcatacatggaagcgaatcaatggactctggaatatccctggacaacagttataaaat |
| | | | | | | ggattatcctgagatgggtttatgtataataattaataataagaattttcataagagcactggaatga |
| | | | | | | catctcggtctggtacagatgtcgatgcagcaaacctcagggaaacattcagaaacttgaaatat |
| | | | | | | gaagtcaggaataaaaatgatcttacacgtgaagaaattgtggaattgatgcgtgatgtttctaaa |
| | | | | | | gaagatcacagcaaaaggagcagttttgtttgtgtgcttctgagccatggtgaagaaggaataat |
| | | | | | | ttttggaacaaatggacctgttgacctgaaaaaaataacaaactttttcagaggggatcgttgtag |
| | | | | | | aagtctaactggaaaacccaaacttttcattattcaggcctgccgtggtacagaactggactgtg |
| | | | | | | gcattgagacagactga |
| 286 | CDS_human_NTCP | Human NTCP | CDS | DNA or | 1050 | atggaggcccacaacgcgtctgccccattcaacttcaccctgccacccaactttggcaagcgc |
| | | (sodium taurocholate | | RNA | | cccacagacctggcactgagcgtcatcctggtgttcatgttgttcttcatcatgctctcgctgggct |
| | | cotransporting | | | | gcaccatggagttcagcaagatcaaggctcacttatggaagcctaaagggctggccatcgccc |
| | | polypeptide) (Homo | | | | tggtggcacagtatggcatcatgcccctcacggcctttgtgctgggcaaggtcttccggctgaa |
| | | sapiens) | | | | gaacattgaggcactggccatcttggtctgtggctgctcacctggagggaacctgtccaatgtct |
| | | | | | | tcagtctggccatgaagggggacatgaacctcagcattgtgatgaccacctgctccaccttctgt |
| | | | | | | gcccttggcatgatgcctctcctcctgtacatctactccagggggatctatgatggggacctgaa |
| | | | | | | ggacaaggtgccctataaaggcatcgtgatatcactggtcctggttctcattccttgcaccatagg |
| | | | | | | gatcgtcctcaaatccaaacggccacaatacatgcgctatgtcatcaagggagggatgatcatc |
| | | | | | | attctcttgtgcagtgtggccgtcacagttctctctgccatcaatgtggggaagagcatcatgtttg |
| | | | | | | ccatgacaccactcttgattgccacctcctccctgatgccttttattggctttctgctgggttatgt |
| | | | | | | tctctctgctctcttctgcctcaatggacggtgcagacgcactgtcagcatggagactggatgccaa |
| | | | | | | aatgtccaactctgttccaccatcctcaatgtggcctttccacctgaagtcattggaccacttttctt |
| | | | | | | ctttcccctcctctacatgattttccagcttggagaagggcttctcctcattgccatattttggtgc |
| | | | | | | tatgagaaattcaagactcccaaggataaaacaaaaatgatctacacagctgccacaactgaaga |
| | | | | | | aacaattccaggagctctgggaaatggcacctacaaaggggaggactgctccccttgcacag |
| | | | | | | cctag |
| 287 | CDS_mouse_ | Human interferon- | CDS | DNA or | 549 | atgaacaacaggtggatcctccacgctgcattcctgctgtgcttctccaccacagccctctccat |
| | IFN_beta_1_ | lambda 3 precursor | | RNA | | caactataagcagctccagctccaagaaaggacaaacattcggaaatgtcaggagctcctgga |
| | precursor | (Homo sapiens) | | | | gcagctgaatggaaagatcaacctcacctacagggctgacttcaagatccctatggagatgac |
| | | | | | | agagaagatgcagaagtcttacactgcctttgccatccaagagatgctccagaatgtctttcttgt |
| | | | | | | cttcagaaacaatttctccagcactgggtggaatgagactattgttgtaaggctcctggatgaact |
| | | | | | | ccaccagcagacagtgtttctgaagacagtattggaggaaaagcaagaggaaagattgacgtg |
| | | | | | | ggagatgtcctctactgctctccacttgaagagctattactggagggtgcaaaggtacctcaaac |
| | | | | | | tcatgaagtacaacagctacgcctggatggtggtccgggcagagatcttcaggaactttctcatc |
| | | | | | | attcgaagacttaccagaaacttccaaaactga |
| 288 | CDS_mouse_ | Mouse interferon- | CDS | DNA or | 582 | atgctcctcctgctgttgcctctgctgctggccgcagtgctgacaagaacccaagctgaccctgt |
| | IFN_lambda_ | lambda 2 precursor | | RNA | | ccccagggccaccaggctcccagtggaagcaaaggattgccacattgctcagttcaagtctct |
| | 2_precursor | (Mus musculus) | | | | gtccccaaaagagctgcaggccttcaaaaaggccaaggatgccatcgagaagaggctgcttg |
| | | | | | | agaaggacctgaggtgcagttcccacctcttccccagggcctgggacctgaagcagctgcag |
| | | | | | | gtccaagagcgccccaaggccttgcaggctgaggtggccctgaccctgaaggtctgggaga |
| | | | | | | acatgactgactcagccctggccaccatcctgggccagcctcttcatacactgagccacattca |
| | | | | | | ctcccagctgcagacctgtacacagcttcaggccacagcagagcccaggtccccgagccgcc |
| | | | | | | gcctctcccgctggctgcacaggctccaggaggcccagagcaaggagacccctggctgcct |
| | | | | | | ggaggcctctgtcacctccaacctgtttcgcctgctcacccgggacctcaagtgtgtggccaat |
| | | | | | | ggagaccagtgtgtctga |
| 289 | CDS_mouse_ | Mouse interferon- | CDS | DNA or | 582 | atgctcctcctgctgttgcctctgctgctggccgcagtgctgacaagaacccaagctgaccctgt |
| | IFN_lambda_ | lambda 3 precursor | | RNA | | ccccagggccaccaggctcccagtggaagcaaaggattgccacattgctcagttcaagtctct |
| | 3_precursor | (Mus musculus) | | | | gtccccaaaagagctgcaggccttcaaaaaggccaagggtgccatcgagaagaggctgcttg |
| | | | | | | agaaggacatgaggtgcagttcccacctcatctccagggcctgggacctgaagcagctgcag |
| | | | | | | gtccaagagcgccccaaggccttgcaggctgaggtggccctgaccctgaaggtctgggaga |
| | | | | | | acataaatgactcagccctgaccaccatcctgggccagcctcttcatacactgagccacattcac |
| | | | | | | tcccagctgcagacctgtacacagcttcaggccacagcagagcccaagcccccgagtcgccg |
| | | | | | | cctctcccgctggctgcacaggctccaggaggcccagagcaaggagactcctggctgcctgg |
| | | | | | | aggactctgtcacctccaacctgtttcaactgctcctccgggacctcaagtgtgtggccagtgga |
| | | | | | | gaccagtgtgtctga |
| 290 | CDS_mouse_ | Mouse interferon- | CDS | DNA or | 600 | atgactccaaagtttttatggctggtggcccttgtggctctatacattccgcccatccaatctctga |
| | IFN_kappa_ | kappa precursor (Mus | | RNA | | actgtgtttacctggatgatagcatcttggaaaatgtgaaacttctgggcagtaccatgaccggct |
| | precursor | musculus) | | | | ttcccttaagatgtctaaaagatatcacagattttaagtttcctaaagagattttgccatacatccag |
| | | | | | | catatgaaaagggagataaacgccgtctcctatcgtatatcctctctggcactaactatcttcaatc |
| | | | | | | ttaaaggctccatccctccagtgacagaggaacactgggaacgtatcagatcgggacttttcaa |
| | | | | | | acaagtgcggcaagctcaagagtgcttcatggacgaggagaaagagaacagggaacatcct |
| | | | | | | cactccgaggacttcctgacagtctacctggagttgggcaagtatttcttcagaatcaaaaagttc |
| | | | | | | ctgataaataagaaatacagtttctgtgcatggaagattgtcacagtggaaataagaagatgtttc |
| | | | | | | attatattttccaagtccagaaaactactcaaaatgatatcagaatcacccaccttcaagcaagaa |
| | | | | | | cttaaatag |
| 291 | CDS_mouse_ | Mouse interleukin-2 | CDS | DNA or | 510 | atgtacagcatgcagctcgcatcctgtgtcacattgacacttgtgctccttgtcaacagcgcaccc |
| | IL2_precursor | precursor (Mus | | RNA | | acttcaagctccacttcaagctctacagcggaagcacagcagcagcagcagcagcagcagca |
| | | musculus) | | | | gcagcagcagcacctggagcagctgttgatggacctacaggagctcctgagcaggatggag |
| | | | | | | aattacaggaacctgaaactccccaggatgctcaccttcaaattttacttgcccaagcaggccac |
| | | | | | | agaattgaaagatcttcagtgcctagaagatgaacttggacctctgcggcatgttctggatttgac |
| | | | | | | tcaaagcaaaagctttcaattggaagatgctgagaatttcatcagcaatatcagagtaactgttgt |
| | | | | | | aaaactaaagggctctgacaacacatttgagtgccaattcgatgatgagtcagcaactgtggtg |
| | | | | | | gactttctgaggagatggatagccttctgtcaaagcatcatctcaacaagccctcaataa |
| 292 | CDS_mouse_ | Mouse interleukin- | CDS | DNA or | 1782 | atggtcagcgttccaacagcctcaccctcggcatccagcagctcctctcagtgccggtccagca |
| | IL12A_P2A_ | 12A and interleukin- | | RNA | | tgtgtcaatcacgctacctcctctttttggccacccttgccctcctaaaccacctcagtttggccag |
| | IL12B_precursors | 12B precursors | | | | ggtcattccagtctctggacctgccaggtgtcttagccagtcccgaaacctgctgaagaccaca |
| | | (bicistronic via P2A | | | | gatgacatggtgaagacggccagagaaaaactgaaacattattcctgcactgctgaagacatc |
| | | sequence) (Mus | | | | gatcatgaagacatcacacgggaccaaaccagcacattgaagacctgtttaccactggaacta |
| | | musculus) | | | | cacaagaacgagagttgcctggctactagagagacttcttccacaacaagagggagctgcctg |
| | | | | | | cccccacagaagacgtctttgatgatgaccctgtgccttggtagcatctatgaggacttgaagat |
| | | | | | | gtaccagacagagttccaggccatcaacgcagcacttcagaatcacaaccatcagcagatcatt |
| | | | | | | ctagacaagggcatgctggtggccatcgatgagctgatgcagtctctgaatcataatggcgaga |
| | | | | | | ctctgcgccagaaacctcctgtgggagaagcagacccttacagagtgaaaatgaagctctgca |
| | | | | | | tcctgcttcacgccttcagcacccgcgtcgtgaccatcaacagggtgatgggctatctgagctc |
| | | | | | | cgccggaagcggagctactaacttcagcctgctgaagcaggctggagacgtggaggagaac |
| | | | | | | cctggacctatgtgtcctcagaagctaaccatctcctggtttgccatcgttttgctggtgtctccact |
| | | | | | | catggccatgtgggagctggagaaagacgtttatgttgtagaggtggactggactcccgatgcc |
| | | | | | | cctggagaaacagtgaacctcacctgtgacacgcctgaagaagatgacatcacctggacctca |
| | | | | | | gaccagagacatggagtcataggctctggaaagaccctgaccatcactgtcaaagagtttctag |
| | | | | | | atgctggccagtacacctgccacaaaggaggcgagactctgagccactcacatctgctgctcc |
| | | | | | | acaagaaggaaaatggaatttggtccactgaaattttaaaaaatttcaaaaacaagactttcctga |
| | | | | | | agtgtgaagcaccaaattactccggacggttcacgtgctcatggctggtgcaaagaaacatgg |
| | | | | | | acttgaagttcaacatcaagagcagtagcagttcccctgactctcgggcagtgacatgtggaat |
| | | | | | | ggcgtctctgtctgcagagaaggtcacactggaccaaagggactatgagaagtattcagtgtcc |
| | | | | | | tgccaggaggatgtcacctgcccaactgccgaggagaccctgcccattgaactggcgttggaa |
| | | | | | | gcacggcagcagaataaatatgagaactacagcaccagcttcttcatcagggacatcatcaaa |
| | | | | | | ccagacccgcccaagaacttgcagatgaagcctttgaagaactcacaggtggaggtcagctg |
| | | | | | | ggagtaccctgactcctggagcactccccattcctacttctccctcaagttctttgttcgaatccag |
| | | | | | | cgcaagaaagaaaagatgaaggagacagaggaggggtgtaaccagaaaggtgcgttcctcg |
| | | | | | | tagagaagacatctaccgaagtccaatgcaaaggcgggaatgtctgcgtgcaagctcaggatc |
| | | | | | | gctattacaattcctcatgcagcaagtgggcatgtgttccctgcagggtccgatcctga |
| 293 | CDS_Syrian_ | Syrian hamster | CDS | DNA or | 543 | atgaccaacaggtggatccttcaagctgcatttctgctgtggtttctccccatatccctgtccatca |
| | hamster_IFN_ | interferon-beta 1 | | RNA | | actacgaggaacttgaggccagacaaagcagaagcaattcagagtgtgaggacctcctgaaa |
| | beta_precursor | precursor | | | | cagctgcgtggacctctctgcctcagagacagaaagttcttcaagatgcctatggaggtgaacc |
| | | (Mesocricetus auratus) | | | | acccagaacagatggagaagagagacactgccttcatcatccaagagatgctccagaatatctt |
| | | | | | | tcttgtcttcaacaacactttctccagcactgggtggaataagaccatcgttgaaagtttcttaggt |
| | | | | | | aaacttcacaatcagatagtctttctgaatgaaaccctaaaggaagtgccagaggatgaaagttt |
| | | | | | | gactacaaggattagcttgaaaagctattactggcagctgggaaggtaccttaaagagaagag |
| | | | | | | gtacagcagctgcgcctggatggtgacccgagcagcagtctcaaggaactttttaatcattaga |
| | | | | | | aggctcacttggatcttccagagctga |
| 294 | CDS_Syrian | Syrian hamster | CDS | DNA or | 639 | atgcccagctcctcacagaccccagagagcagcatgaagccagctgggggccgcctgctcct |
| | hamster_IFN_ | interferon-lambda 3 | | RNA | | cctgctgctgctgctgctgctgctggccgcggtgctgacgggagcccgcgctcatcctgtgcc |
| | lambda_3_ | precursor | | | | cagggccaccaggctcccaccagatgcaaaggattgtcacatagcccggttcaagtctctgcc |
| | precursor | (Mesocricetus auratus) | | | | cccgcaagagctgcaggccttcaagaaggccaaggatgccatagaagagtggctgctcaag |
| | | | | | | aaggacgtcaggtgcagctcccgcctcttccccagggcctgggacctgaagcagctgcaggt |
| | | | | | | ccaggagcgccccgaggccttgcaggctgagctgaccctcacactgaaggtcctggagaac |
| | | | | | | gtgactgacccgggcctgggccccatcctggaccagcctcttctcaccctgagccacatccact |
| | | | | | | cacagctgcaggcctgtactcagcctcagcctgcagcagagcccaggccccccagccgccg |
| | | | | | | cctctcccgctggctgcacaggctccaggaggcccaggagaaggagacccccggctgcctg |
| | | | | | | gaagtctctgtcacttccaacctcttccgcctgctcatccgggacctgaagtgtgtggccagtgg |
| | | | | | | agaccagtgtgactga |
| 295 | CDS_ferret_IFN_ | Domestic ferret | CDS | DNA or | 561 | atgaccagcaggtgtatcctccaaactgctctcctggtatatttctccaccacggttcttgccatga |
| | beta_1_precursor | interferon beta 1 | | RNA | | actataacttacttcgattccaactaagcagcagcagtgtggagtgccaggagctccttctacag |
| | | precursor (Mustela | | | | ttgaatggaacatctaaagactgcctcaaggacaggatgaacttcaagatccctgaggagatcc |
| | | putorius furo) | | | | agaaatcccagaagttccagaaggaggacatcgtattggtcacccttgagatgttccaaaagac |
| | | | | | | ttctgatattttcaggagaaatctctcaagcacgggatggaatgagagcattgtcgagaacctcct |
| | | | | | | tgccacactccactggcagaaggaacatctggaggaaatcctggaggacatcatgcaggagg |
| | | | | | | aaaacgtcacttgggaccacaggacccttctgcacctgaagagatattatctaaggattgtgcg |
| | | | | | | gtacctgaaagcgaaggagttcagcgtctgtgcctggacaatagtgcaagcagaaatcctcag |
| | | | | | | gagctttttcttccttgataaacttacagattctctccctaactga |
| 296 | CDS_cotton | Cotton rat interferon- | CDS | DNA or | 573 | atgtccaggtcatgtgctttcctgacatttctgctggtgatgagctactggacaacctgctctctgg |
| | rat_IFN_alpha_ | alpha precursor | | RNA | | gatgtgacctgcctcagattcataacctcaggaacaacacagccttcacactcctggtgcaaatg |
| | precursor | (Sigmodon hispidus) | | | | aggacactcttccctctctcctgcctgaaggacagaaaggactttgcatcccctctggagaaggt |
| | | | | | | ggttgcccagcagctccagaaggctcaagccatccttgtctcaagtgaggtgatccaacagatc |
| | | | | | | ctgaacattttcacctcaaatgaatcatctgctgcttgggagacaaccctcctggaaacattctgt |
| | | | | | | gacagcctctaccagaagctcagagacctacaagactgtctgatgcagcaggaggaggtgaa |
| | | | | | | ggaacctaccctgagcgaggaagactcgctggggactgtgaggcagtacttccacaggatca |
| | | | | | | ctgtctacctgaaagagaggaaacacagcccctgtgcctgggaagtggtcagagcagaagtct |
| | | | | | | ggagagccctgtcttcctcagtcaaattgctggcaagatggagtgaggagaagaagtaa |
| 297 | CDS_cotton_ | Cotton rat interferon- | CDS | DNA or | 546 | atggtcaacagatggattctccaacctgcattcctgctgtggtttctccctacagccctctccatca |
| | rat_IFN_beta_ | beta 1 precursor | | RNA | | actacaagcagcttgagctcagacaaaggagcagcatttcagaatgtctagagctcttgaatca |
| | 1_precursor | (Sigmodon hispidus) | | | | gatgaatggaccgctctgtctcagctatagacagaacttcaagattcctatggaagtgaagcatc |
| | | | | | | caagacagctagagaaggatgaagctgtctccgtcattgaagtgatgctccagaatatcttccac |
| | | | | | | atcttccgcagcaattcctccagcaccgggtggaataaaaacatcattgaaagcttcttggataa |
| | | | | | | actccgtaacgagatggactttctagaggaaataccagagaatgaaagtttgactccaaggaac |
| | | | | | | tttaccaccattctacccttgaaaagctattataagaggatgcaagagtatcttgaaaaccatgaat |
| | | | | | | acagcagctgttcctggatagtagtccgaacagaaatccgcaggaacttttccatcatgggaag |
| | | | | | | acttattaagatcttccagaactga |
| 298 | CDS_cotton_ | Cotton rat interferon- | CDS | DNA or | 603 | atgaagccagaaagagctgggggccacgggctcctcctcctgctgctggccccggtgctggc |
| | rat_IFN_lambda_ | lambda 2 precursor | | RNA | | gggaacccgcgctaaccctgtgcccagggccaccaggctgccaccagatacaaagcattgc |
| | 2_precursor | (Sigmodon hispidus) | | | | cacctagcccagttcaagtccctgtccccgcaagcgctgcagaacttcaggaaggccaaggat |
| | | | | | | gccatagaagagtggcgtctcaagaaggaggacatcaggtgcagctcccgcctcttccccag |
| | | | | | | ggcgtgggacctgcagcagctgcaggtccaggagcgccccatggccctgaaggctgagctg |
| | | | | | | gccctgacactgagggtcctggagaacatgactgactcagccctggatcccatcctggaccag |
| | | | | | | ccccttcgcacactgcgccacatccaatcacagctgcaggcctgtactcagcctcagctcacag |
| | | | | | | cagagcccaggcccccaagtcgccgcctctctcgctggctgcaaaggctgcaggaagccca |
| | | | | | | gaggaaggagacccccggcagcctggaggcctgtgtcacatacaacctcttccgcctgctga |
| | | | | | | ctcgggacctgaagtgtgtggccagtggagaccagtgtgcctaa |
| 299 | CDS_BST2_ | BST-2 (Tetherin) | CDS | DNA or | 543 | atggcatctacttcgtatgactattgcagagtgcccatggaagacggggataagcgctgtaagc |
| | Tetherin_CD317 | (CD317) precursor | | RNA | | ttctgctggggataggaattctggtgctcctgatcatcgtgattctgggggtgcccttgattatcttc |
| | | (Homo sapiens) | | | | accatcaaggccaacagcgaggcctgccgggacggccttcgggcagtgatggagtgtcgca |
| | | | | | | atgtcacccatctcctgcaacaagagctgaccgaggcccagaagggctttcaggatgtggagg |
| | | | | | | cccaggccgccacctgcaaccacactgtgatggccctaatggcttccctggatgcagagaag |
| | | | | | | gcccaaggacaaaagaaagtggaggagcttgagggagagatcactacattaaaccataagct |
| | | | | | | tcaggacgcgtctgcagaggtggagcgactgagaagagaaaaccaggtcttaagcgtgaga |
| | | | | | | atcgcggacaagaagtactaccccagctcccaggactccagctccgctgcggcgccccagct |
| | | | | | | gctgattgtgctgctgggcctcagcgctctgctgcagtga |
| 300 | CDS_SARS_ | Sudden Acute | CDS | DNA or | 336 | atgtttgtttttcttgttttattgccactagtctctagtgttgtaaacattcaaaaagaaattgaccgc |
| | Spike_truncation | Respiratory Syndrome | | RNA | | ctcaatgaggttgccaagaatttaaatgaatctctcatcgatctccaagaacttggaaagtatgagca |
| | | Coronavirus 2 (SARS- | | | | gtatataaaatggccatggtacatttggctaggttttatagctggcttgattgccatagtaatggtg |
| | | CoV-2) Spike | | | | acaattatgctttgctgtatgaccagttgctgtagttgtctcaagggctgttgttcttgtggatcct |
| | | truncation protein | | | | gctgcaaatttgatgaagacgactctgagccagtgctcaaaggagtcaaattaaaatacacataa |
| 301 | rcCDS_GDura | GDura | rcCDS | DNA or | 558 | ttagtcaccaccggcccccttgatcttgtccacctggccctggatcttgctggcaaaggtcgcac |
| | | | | RNA | | agcgttgcggcagccacttcttgagcaggtcagaacactgcacgttggcaagccctttgaggc |
| | | | | | | agccagttgtgcagtccacacacagatcgacctgtgcgatgaactgctccaagggctccaagt |
| | | | | | | ccttgaacccaggaatctcaggaatgtcgacgatcgcctcgcctatgccgccctgtgcggactc |
| | | | | | | tttgtcgccttcgtaggtgtggcagcgtcctgggatgaacttcttcatcttgggcgtgcacttgatg |
| | | | | | | tgggacaggcagatcagacagcccctggtgcagccagctttccgggcattggcttccaactctt |
| | | | | | | tgagcacctccagcggcagcttcttgccgggcaacttcccgcggtcagcatcgagatccgtgg |
| | | | | | | tcgcgaagttgctggccacggccacgatgttgaagtcttcgttgttctcggtgggcttggcctcg |
| | | | | | | gccacagcgatgcagatcagggcaaacagaactttgactcccat |
| 302 | rcCDS_EGFP | EGFP | rcCDS | DNA or | 722 | ttacacttgtacagctcgtccatgccgagagtgatcccggcggcggtcacgaactccagcagg |
| | | | | RNA | | accatgtgatcgcgcttctcgttggggtctttgctcagggcggactgggtgctcaggtagtggtt |
| | | | | | | gtcgggcagcagcacggggccgtcgccgatgggggtgttctgctggtagtggtcggcgagct |
| | | | | | | gcacgctgccgtcctcgatgttgtggcggatcttgaagttcaccttgatgccgttcttctgcttgtc |
| | | | | | | ggccatgatatagacgttgtggctgttgtagttgtactccagcttgtgccccaggatgttgccgtc |
| | | | | | | ctccttgaagtcgatgcccttcagctcgatgcggttcaccagggtgtcgccctcgaacttcacct |
| | | | | | | cggcgcgggtcttgtagttgccgtcgtccttgaagaagatggtgcgctcctggacgtagccttc |
| | | | | | | gggcatggcggacttgaagaagtcgtgctgcttcatgtggtcggggtagcggctgaagcactg |
| | | | | | | cacgccgtaggtcagggtggtcacgagggtgggccagggcacgggcagcttgccggtggtg |
| | | | | | | cagatgaacttcagggtcagcttgccgtaggtggcatcgccctcgccctcgccggacacgctg |
| | | | | | | aacttgtggccgtttacgtcgccgtccagctcgaccaggatgggcaccaccccggtgaacagc |
| | | | | | | tcctcgcccttgctcaccat |
| 303 | rcCDS_Rluc8 | Rluc8 | rcCDS | DNA or | 930 | ttactgctcgttcttcagcactctctccacgaagctcttgatgtacttgcccatctcgtcgggggcg |
| | | | | RNA | | tcctcctggaggaagtgcaggcccttcaccttcacgaactcggtgttggggaacttcttggcgc |
| | | | | | | cctccacgatggcgttgctgaagaagccggggtcgccctcgatgaacagcttgggcaggtcgt |
| | | | | | | cgctggctctcaggtaggcgttgtagtttctcacgatctgcaccacgtcgggcttgccgcccttc |
| | | | | | | accagggggatctctctgggccagctcagggtgggtcttctcacctcgcccttctccttgaagg |
| | | | | | | gctccaggtaggcggcgaactcctcgggctccagctttctcatgatcttgctgggcagcacggt |
| | | | | | | ctccacgaagaagttgttctccagcaccatcttctcgccctcctcgctcttgatcagggcgatgtc |
| | | | | | | ctcctcgatgtctggccactcgtcccagctctcgatcacgtccaccacgctctccatgtgcacga |
| | | | | | | tggccttgatcctgtcctggtgctcgtaggcgtagtggaaggccagggcggcgccccagtcgt |
| | | | | | | ggcccacgaagatgatcttcttgggcaggttcagaagctcgaaccaggcggtcaggtacttgta |
| | | | | | | gtggtccagcagcctgtagctgccgttgccgctcttgccgctcttgcccatgccgatcagatcg |
| | | | | | | gggatgatgcacctggccacgggctcgatgtggggcaccacgtgcctccacaggtagctgct |
| | | | | | | agtggcgttgccgtgcaggaagatcacggcgttctcggcgtgcttctcgctgtcgtagtagttga |
| | | | | | | tgaagctgtccagcacgttcatctgcttgcacctggcccaccactgggggccggtgatcatcct |
| | | | | | | cttcctctgctcggggtcgtacaccttcat |
| 304 | rcCDS_ | mNeonGreen | rcCDS | DNA or | 711 | ttatttgtacagctcgtccattcccattacatcggtgaacgctttctgccattccttgaagttaagctc |
| | mNeonGreen | | | RNA | | tgttttgctatgcttaagctcggtcttgcggaaaacgtacatgggctgattcttaaggtagttggct |
| | | | | | | gccataggctttgcgaaggtataagttgtcctggcggtgctccgatatcgcttgccgttacccgta |
| | | | | | | gtgtagctccacttgaatgtgctaatgattgttttgtcatttggataggtttttttagaccgacaccag |
| | | | | | | tccgcggctgtaaggctattggtcataactggaccatcggccggaaaccctgtgccttttacttg |
| | | | | | | cgcctcacctttgatatgggagccttcgtatgtgtacctataattaacagtgagagacgcaccatc |
| | | | | | | ctcaaactgcatggttcgatgcacttgataacctgatccgtccaccatagcggcctgaaaggga |
| | | | | | | ctcatgccgtcgggataagggaggtattgatggaatccgtagccgatatggggaaccaatatcc |
| | | | | | | acggggagaattgcaagtcacccttagtactcttaagattcaattcttcgtatccgtcgttaggatt |
| | | | | | | gccggtgccttggccgaccatatcgaaatccaccccgtttattgatccaaatatatgcaattcatg |
| | | | | | | tgtagccgggagtgaagccatattgtcctcctcgcccttgcttaccat |
| 305 | rcCDS_LacZ | LacZ | rcCDS | DNA or | 3075 | ttatttttgacaccagaccaactggtaatggtagcgaccggcgctcagctggaattccgccgata |
| | | | | RNA | | ctgacgggctccaggagtcgtcgccaccaatccccatatggaaaccgtcgatattcagccatgt |
| | | | | | | gccttcttccgcgtgcagcagatggcgatggctggtttccatcagttgctgttgactgtagcggct |
| | | | | | | gatgttgaactggaagtcgccgcgccactggtgtgggccataattcaattcgcgcgtcccgcag |
| | | | | | | cgcagaccgttttcgctcgggaagacgtacggggtatacatgtctgacaatggcagatcccag |
| | | | | | | cggtcaaaacaggcggcagtaaggcggtcgggatagttttcttgcggccctaatccgagccag |
| | | | | | | tttacccgctctgctacctgcgccagctggcagttcaggccaatccgcgccggatgcggtgtat |
| | | | | | | cgctcgccacttcaacatcaacggtaatcgccatttgaccactaccatcaatccggtaggttttcc |
| | | | | | | ggctgataaataaggttttcccctgatgctgccacgcgtgagcggtcgtaatcagcaccgcatc |
| | | | | | | agcaagtgtatctgccgtgcactgcaacaacgctgcttcggcctggtaatggcccgccgccttc |
| | | | | | | cagcgttcgacccaggcgttagggtcaatgcgggtcgcttcacttacgccaatgtcgttatccag |
| | | | | | | cggtgcacgggtgaactgatcgcgcagcggcgtcagcagttgttttttatcgccaatccacatct |
| | | | | | | gtgaaagaaagcctgactggcggttaaattgccaacgcttattacccagctcgatgcaaaaatc |
| | | | | | | catttcgctggtggtcagatgcgggatggcgtgggacgcggcggggagcgtcacactgaggt |
| | | | | | | tttccgccagacgccactgctgccaggcgctgatgtgcccggcttctgaccatgcggtcgcgtt |
| | | | | | | cggttgcactacgcgtactgtgagccagagttgcccggcgctctccggctgcggtagttcagg |
| | | | | | | cagttcaatcaactgtttaccttgtggagcgacatccagaggcacttcaccgcttgccagcggct |
| | | | | | | taccatccagcgccaccatccagtgcaggagctcgttatcgctatgacggaacaggtattcgct |
| | | | | | | ggtcacttcgatggtttgcccggataaacggaactggaaaaactgctgctggtgttttgcttccgt |
| | | | | | | cagcgctggatgcggcgtgcggtcggcaaagaccagaccgttcatacagaactggcgatcgt |
| | | | | | | tcggcgtatcgccaaaatcaccgccgtaagccgaccacgggttgccgttttcatcatatttaatca |
| | | | | | | gcgactgatccacccagtcccagacgaagccgccctgtaaacggggatactgacgaaacgc |
| | | | | | | ctgccagtatttagcgaaaccgccaagactgttacccatcgcgtgggcgtattcgcaaaggatc |
| | | | | | | agcgggcgcgtctctccaggtagcgaaagccattttttgatggaccatttcggcacagccggga |
| | | | | | | agggctggtcttcatccacgcgcgcgtacatcgggcaaataatatcggtggccgtggtgtcgg |
| | | | | | | ctccgccgccttcatactgcaccggggggaaggatcgacagatttgatccagcgatacagcg |
| | | | | | | cgtcgtgattagcgccgtggcctgattcattccccagcgaccagatgatcacactcgggtgatta |
| | | | | | | cgatcgcgctgcaccattcgcgttacgcgttcgctcatcgccggtagccagcgcggatcatcg |
| | | | | | | gtcagacgattcattggcaccatgccgtgggtttcaatattggcttcatccaccacatacaggcc |
| | | | | | | gtagcggtcgcacagcgtgtaccacagcggatggttcggataatgcgaacagcgcacggcgt |
| | | | | | | taaagttgttctgcttcatcagcaggatatcctgcaccatcgtctgctcatccatgacctgaccatg |
| | | | | | | cagaggatgatgctcgtgacggttaacgcctcgaatcagcaacggcttgccgttcagcagcag |
| | | | | | | cagaccattttcaatccgcacctcgcggaaaccgacatcgcaggcttctgcttcaatcagcgtg |
| | | | | | | ccgtcggcggtgtgcagttcaaccaccgcacgatagagattcgggatttcggcgctccacagtt |
| | | | | | | tcgggttttcgacgttcagacgtagtgtgacgcgatcggcataaccaccacgctcatcgataattt |
| | | | | | | caccgccgaaaggcgcggtgccgctggcgacctgcgtttcaccctgccataaagaaactgtta |
| | | | | | | cccgtaggtagtcacgcaactcgccgcacatctgaacttcagcctccagtacagcgcggctga |
| | | | | | | aatcatcattaaagcgagtggcaacatggaaatcgctgatttgtgtagtcggtttatgcagcaac |
| | | | | | | gagacgtcacggaaaatgccgctcatccgccacatatcctgatcttccagataactgccgtcact |
| | | | | | | ccagcgcagcaccatcaccgcgaggcggttttctccggcgcgtaaaaatgcgctcaggtcaa |
| | | | | | | attcagacggcaaacgactgtcctggccgtaaccgacccagcgcccgttgcaccacagatga |
| | | | | | | aacgccgagttaacgccatcaaaaataattcgcgtctggccttcctgtagccagctttcatcaac |
| | | | | | | attaaatgtgagcgagtaacaacccgtcggattctccgtgggaacaaacggcggattgaccgt |
| | | | | | | aatgggataggtcacgttggtgtagatgggcgcatcgtaaccgtgcatctgccagtttgagggg |
| | | | | | | acgacgacagtatcggcctcaggaagatcgcactccagccagctttccggcaccgcttctggt |
| | | | | | | gccggaaaccaggcaaagcgccattcgccattcaggctgcgcaactgttgggaagggcgatc |
| | | | | | | ggtgcgggcctcttcgctattacgccagctggcgaaagggggatgtgctgcaaggcgattaag |
| | | | | | | ttgggtaacgccagggttttcccagtcacgacgttgtaaaacgacggccagtgaatccgtaatc |
| | | | | | | atggtcat |
| 306 | rcCDS_human_ | Human interferon-beta | rcCDS | DNA or | 564 | tcagtttcggaggtaacctgtaagtctgttaatgaagtaaaagttccttaggatttccactctgacta |
| | IFN_beta_ | precursor (Homo | | RNA | | tggtccaggcacagtgactgtactccttggccttcaggtaatgcagaatcctcccataatatctttt |
| | 1_precursor | sapiens) | | | | caggtgcagactgctcatgagttttcccctggtgaaatcttctttctccagtttttcttccaggactgt |
| | | | | | | cttcagatggtttatctgatgatagacattagccaggaggttctcaacaatagtctcattccagcca |
| | | | | | | gtgctagatgaatcttgtctgaaaatagcaaagatgttctggagcatctcatagatggtcaatgcg |
| | | | | | | gcgtcctccttctggaactgctgcagctgcttaatctcctcagggatgtcaaagttcatcctgtcct |
| | | | | | | tgaggcagtattcaagcctcccattcaattgccacaggagcttctgacactgaaaattgctgcttc |
| | | | | | | tttgtaggaatccaagcaagttgtagctcatggaaagagctgtagtggagaagcacaacagga |
| | | | | | | gagcaatttggaggagacacttgttggtcat |
| 307 | rcCDS_human_ | Human interferon- | rcCDS | DNA or | 603 | tcaggtggactcagggtgggttgacgttctcagacacaggttcccatcggccacatatttgaggt |
| | IFN_lambda_ | lambda precursor | | RNA | | ctcgcgtgaggaggcggaagaggttgaaggtgacagatgcctccaggcagccagcggactc |
| | precursor | (Homo sapiens) | | | | ctttttgggggcctcctggagccggtgcagccagtggtggaggcggccccggggcctgggc |
| | | | | | | cctgctgtgggctgaggctggatacaggcctggagctgggagaggatgtggtgcagggtgtg |
| | | | | | | aaggggctggtctaggacgtcctccagggctgggccagcagcggcctccaggaccttcagc |
| | | | | | | gtcagggccagctcagcctccaaggccacagggcgctccctcacctggagaagcctcaggtc |
| | | | | | | ccaattcccggggaagacaggagagctgcaactccagtttttcagcttgagtgactcttccaag |
| | | | | | | gcgtccctggccttcttgaagctcgctagctcctgtggtgacagagatttgaacctgccaatgtg |
| | | | | | | gcagcccttcccagttgtggtgggcttggaagtggggacagggcctgccacggccaagccta |
| | | | | | | gcaccaaagtcaccagcaccacggtccaagctgcagccat |
| 308 | rcCDS_human_ | Human interferon- | rcCDS | DNA or | 591 | tcagacacacaggtccccgctggcaacacaattcaggtctcgcgtgaggaggcggaagaggt |
| | IFN_lambda_ | lambda 3 precursor | | RNA | | tgaaggtgacagaggcctcgaggcagccaggggactccttttttggggcctcctggagccggt |
| | 3_precursor | (Homo sapiens) | | | | gcagccaatggtggaggcggccccgggtcctgggccctgccgtgggctgaggctggataca |
| | | | | | | ggcccggagctgggagaggatatggtgcagggtgtgaaggggctggtccaagacatccccc |
| | | | | | | agggctgggtcagtgtcagcggtggcctccagaaccttcagcgtcagggccagctcagcctc |
| | | | | | | caaagccacggggcgctccctcacctgcagctgcctcaggtcccaggtcctggggaagagg |
| | | | | | | cgggagcggcacttgcagtccttcagcagaagcgactcttctaaggcatctttggccctcttaaa |
| | | | | | | ggcctgcagctcctgtggagacagggacttgaactgggctatgtggcagccccttgcatccgg |
| | | | | | | gagagccccgcggagcctggcgacaggaactgctccagtcacggtcagcactgcggccatc |
| | | | | | | agcaccagcactggcatgcagtccccggtcat |
| 309 | rcCDS_human_ | Human interleukin-2 | rcCDS | DNA or | 462 | tcaagtcagtgttgagatgatgctttgacaaaaggtaatccatctgttcagaaattctacaatggtt |
| | IL2_precursor | precursor (Homo | | RNA | | gctgtctcatcagcatattcacacatgaatgttgtttcagatccctttagttccagaactattacgttg |
| | | sapiens) | | | | atattgctgattaagtccctgggtcttaagtgaaagtttttgctttgagctaaatttagcacttcctcc |
| | | | | | | agaggtttgagttcttcttctagacactgaagatgtttcagttctgtggccttcttgggcatgtaaaa |
| | | | | | | cttaaatgtgagcatcctggtgagtttgggattcttgtaattattaattccattcaaaatcatctgtaa |
| | | | | | | atccagcagtaaatgctccagttgtagctgtgttttctttgtagaacttgaagtaggtgcactgtttg |
| | | | | | | tgacaagtgcaagacttagtgcaatgcaagacaggagttgcatcctgtacat |
| 310 | rcCDS_human_ | Human interleukin- | rcCDS | DNA or | 1710 | ctaactgcagggcacagatgcccattcgctccaagatgagctatagtagcggtcctgggcccg |
| | IL12A_P2A_ | 12A and interleukin- | | RNA | | cacgctaatgctggcatttttgcggcagatgaccgtggctgaggtcttgtccgtgaagactctatc |
| | IL12B_ | 12B precursors (bi- | | | | tttcttttctctcttgctcttgccctggacctgaacgcagaatgtcagggagaagtaggaatgtgg |
| | precursors | cistronic via P2A site) | | | | agtactccaggtgtcagggtactcccagctgacctccacctgccgagaattctttaatggcttca |
| | | (Homo sapiens) | | | | gctgcaagttcttgggtgggtcaggtttgatgatgtccctgatgaagaagctgctggtgtagttttc |
| | | | | | | atacttgagcttgtgaacggcatccaccatgacctcaatgggcagactctcctcagcagctggg |
| | | | | | | caggcactgtcctcctggcactccactgagtactcatactccttgttgtcccctctgactctctctg |
| | | | | | | cagagagtgtagcagctccgcacgtcaccccttgggggtcagatgagcctctgctgcttttgac |
| | | | | | | actgaatgtcaaatcagtactgattgtcgtcagccaccagcaggtgaaacgtccagaataattctt |
| | | | | | | ggcctcgcatcttagaaaggtcttatttttgggttctttctggtcctttaaaatatcagtggaccaaat |
| | | | | | | tccatcttcctttttgtgaagcagcaggagcgaatggcttagaacctcgcctcctttgtgacaggt |
| | | | | | | gtactggccagcatctccaaactctttgacttggatggtcagggttttgccagagcctaagacctc |
| | | | | | | actgctctggtccaaggtccaggtgataccatcttcttcaggggtgtcacaggtgaggaccacc |
| | | | | | | atttctccaggggcatccggataccaatccaattctacgacataaacatctttcttcagttcccatat |
| | | | | | | ggccacgaggggagatgccagaaaaaccagggaaaaccaagagatgaccaactgctggtg |
| | | | | | | acacataggtccagggttctcctccacgtctccagcctgcttcagcaggctgaagttagtagctc |
| | | | | | | cgcttccggaagcattcagatagctcatcactctatcaatagtcactgcccgaattctgaaagcat |
| | | | | | | gaagaagtatgcagagcttgattttagttttataaaaatccggttcttcaagggaggatttttgtggc |
| | | | | | | acagtctcactgttgaaattcagggcctgcatcagctcatcaataactgccagcatgttttgatcta |
| | | | | | | gaaagatctgcctcttaggatccatcagaagctttgcattcatggtcttgaactccacctggtacat |
| | | | | | | cttcaagtcttcataaatactactaaggcacagggccatcataaaagaggtctttctggaggcca |
| | | | | | | ggcaactcccattagttatgaaagaggtctctctggaatttaggcaactctcattcttggttaattcc |
| | | | | | | aatggtaaacaggcctccactgtgctggttttatcttttgtgatatcttcatgatcaatctcttcagaa |
| | | | | | | gtgcaagggtaaaattctagagtttgtctggccttctggagcatgttgctgacggccctcagcag |
| | | | | | | gttttgggagtggtgaaggcatgggaacattcctgggtctggagtggccacggggaggtttctg |
| | | | | | | gccaaactgaggtggtccaggaggaccagggtagccacaaggaggaggctgcgcgctgga |
| | | | | | | cacat |
| 311 | rcCDS_human_ | Human interleukin-1 | rcCDS | DNA or | 534 | ctactcgtcctcctggaagtagaatttggtgaccatgacgccttcgtcaggcatattggtgaggct |
| | IL1RN_isoform_ | receptor antagonist | | RNA | | gacgggctggtcagcttccatcgctgtgcagaggaaccaaccggggcaggcggcagactca |
| | 1_precursor | precursor (Homo | | | | aaactggtggtggggccactgtctgagcggatgaaggcgaagcgcttgtcctgctttctgttctc |
| | | sapiens) | | | | gctcaggtcagtgatgttaactgcctccagctggagtctggtctcatcaccagacttgacacagg |
| | | | | | | acaggcacatcttccctccatggattcccaagaacagagcatgaggctcaatgggtaccacatc |
| | | | | | | tatcttttcttctaaattgacatttggtccttgcaagtatccagcaactagttggttgttcctcagata |
| | | | | | | gaaggtcttctggttaacatcccagattctgaaggcttgcatcttgctggattttctcccagagggtc |
| | | | | | | ggcagatcgtctctgaatggaacaggaagaggaggagagtgattaggtgactgcggaggcct |
| | | | | | | ctgcagatttccat |
| 312 | rcCDS_human_ | Human tumor necrosis | rcCDS | DNA or | 702 | tcacagggcaatgatcccaaagtagacctgcccagactcggcaaagtcgagatagtcgggcc |
| | TNF_precursor | factor precursor | | RNA | | gattgatctcagcgctgagtcggtcacccttctccagctggaagacccctcccagatagatggg |
| | | (Homo sapiens) | | | | ctcataccagggcttggcctcagccccctctggggtctccctctggcaggggctcttgatggca |
| | | | | | | gagaggaggttgaccttggtctggtaggagacggcgatgcggctgatggtgtgggtgaggag |
| | | | | | | cacatgggtggaggggcagccttggcccttgaagaggacctgggagtagatgaggtacagg |
| | | | | | | ccctctgatggcaccaccagctggttatctctcagctccacgccattggccaggagggcattgg |
| | | | | | | cccggcggttcagccactggagctgcccctcagcttgagggtttgctacaacatgggctacag |
| | | | | | | gcttgtcactcggggttcgagaagatgatctgactgcctgggccagagggctgattagagaga |
| | | | | | | ggtccctggggaactcttccctctgggggccgatcactccaaagtgcagcaggcagaaaagc |
| | | | | | | gtggtggcgcctgccacgatcaggaaggagaagaggctgaggaacaagcaccgcctggag |
| | | | | | | ccctggggcccccctgtcttcttggggagcgcctcctcggccagctccacgtcccggatcatg |
| | | | | | | ctttcagtgctcat |
| 313 | rcCDS_human_ | Human activated | rcCDS | DNA or | 873 | tcagtctgtctcaatgccacagtccagttctgtaccacggcaggcctgaataatgaaaagtttggg |
| | revCaspase3 | Caspase 3 (Homo | | RNA | | ttttccagttagacttctacaacgatcccctctgaaaaagtttgttatttttttcaggtcaacaggtc |
| | | sapiens) | | | | catttgttccaaaaattattccttcttcaccatggctcagaagcacacaaacaaaactgctccttttg |
| | | | | | | ctgtgatcttctttagaaacatcacgcatcaattccacaatttcttcacgtgtaagatcatttttattc |
| | | | | | | ctgacttcatatttcaagtttctgaatgtttccctgaggtttgctgcatcgacatctgtaccagaccga |
| | | | | | | gatgtcattccagtgctcttatgaaaattcttattattaattattatacataaacccatctcaggataa |
| | | | | | | tccattttataactgttgtccagggatattccagagtccattgattcgcttccatgtatgatctttggt |
| | | | | | | tccaaatttttaatggattttgaatccactgagttttcagtgttctccatgggggatcccccatcaac |
| | | | | | | ttcatcgtgataaaaatagagttcttttgtgagcatggaaacaatacatggaatctgtttctttgcat |
| | | | | | | gaaaagtagcgtcaaaggaaaaggactcaaattctgttgccacctttcggttaacccgggtaagaat |
| | | | | | | gtgcataaattcaagcttgtcggcatactgtttcagcatggcacaaagcgactggatgaaccag |
| | | | | | | gagccatcctttgaatttcgccaagaataataaccaggtgctgtggagtatgcatacaagaagtc |
| | | | | | | ggcctccactggtattttatgacacgccatgtcatcatcaacaccactgtctgtctcaatcat |
| 314 | rcCDS_human_ | Human NTCP | rcCDS | DNA or | 1050 | ctaggctgtgcaaggggagcagtcctcccctttgtaggtgccatttcccagagctcctggaattg |
| | NTCP | (sodium taurocholate | | RNA | | tttcttcagttgtggcagctgtgtagatcatttttgttttatccttgggagtcttgaatttctcatag |
| | | cotransporting | | | | caccaaaatatggcaatgaggagaagcccttctccaagctggaaaatcatgtagaggaggggaaa |
| | | polypeptide) (Homo | | | | gaagaaaagtggtccaatgacttcaggtggaaaggccacattgaggatggtggaacagagttg |
| | | sapiens) | | | | gacattttggcatccagtctccatgctgacagtgcgtctgcaccgtccattgaggcagaagaga |
| | | | | | | gcagagagaacataacccagcagaaagccaataaaaggcatcagggaggaggtggcaatc |
| | | | | | | aagagtggtgtcatggcaaacatgatgctcttccccacattgatggcagagagaactgtgacgg |
| | | | | | | ccacactgcacaagagaatgatgatcatccctcccttgatgacatagcgcatgtattgtggccgt |
| | | | | | | ttggatttgaggacgatccctatggtgcaaggaatgagaaccaggaccagtgatatcacgatgc |
| | | | | | | ctttatagggcaccttgtccttcaggtccccatcatagatccccctggagtagatgtacaggagg |
| | | | | | | agaggcatcatgccaagggcacagaaggtggagcaggtggtcatcacaatgctgaggttcat |
| | | | | | | gtcccccttcatggccagactgaagacattggacaggttccctccaggtgagcagccacagac |
| | | | | | | caagatggccagtgcctcaatgttcttcagccggaagaccttgcccagcacaaaggccgtgag |
| | | | | | | gggcatgatgccatactgtgccaccagggcgatggccagccctttaggcttccataagtgagc |
| | | | | | | cttgatcttgctgaactccatggtgcagcccagcgagagcatgatgaagaacaacatgaacac |
| | | | | | | caggatgacgctcagtgccaggtctgtggggcgcttgccaaagttgggtggcagggtgaagtt |
| | | | | | | gaatggggcagacgcgttgtgggcctccat |
| 315 | rcCDS_mouse_ | Human interferon- | rcCDS | DNA or | 549 | tcagttttggaagtttctggtaagtcttcgaatgatgagaaagttcctgaagatctctgcccggacc |
| | IFN_beta_ | lambda 3 precursor | | RNA | | accatccaggcgtagctgttgtacttcatgagtttgaggtacctttgcaccctccagtaatagctct |
| | 1_precursor | (Homo sapiens) | | | | tcaagtggagagcagtagaggacatctcccacgtcaatctttcctcttgcttttcctccaatactgt |
| | | | | | | cttcagaaacactgtctgctggtggagttcatccaggagccttacaacaatagtctcattccaccc |
| | | | | | | agtgctggagaaattgtttctgaagacaagaaagacattctggagcatctcttggatggcaaag |
| | | | | | | gcagtgtaagacttctgcatcttctctgtcatctccatagggatcttgaagtcagccctgtaggtga |
| | | | | | | ggttgatctttccattcagctgctccaggagctcctgacatttccgaatgtttgtcctttcttggagc |
| | | | | | | tggagctgcttatagttgatggagagggctgtggtggagaagcacagcaggaatgcagcgtgg |
| | | | | | | aggatccacctgttgttcat |
| 316 | rcCDS_mouse_ | Mouse interferon- | rcCDS | DNA or | 582 | tcagacacactggtctccattggccacacacttgaggtcccgggtgagcaggcgaaacaggtt |
| | IFN_lambda_ | lambda 2 precursor | | RNA | | ggaggtgacagaggcctccaggcagccaggggtctccttgctctgggcctcctggagcctgt |
| | 2_precursor | (Mus musculus) | | | | gcagccagcgggagaggcggcggctcggggacctgggctctgctgtggcctgaagctgtgt |
| | | | | | | acaggtctgcagctgggagtgaatgtggctcagtgtatgaagaggctggcccaggatggtgg |
| | | | | | | ccagggctgagtcagtcatgttctcccagaccttcagggtcagggccacctcagcctgcaagg |
| | | | | | | ccttggggcgctcttggacctgcagctgcttcaggtcccaggccctggggaagaggtgggaa |
| | | | | | | ctgcacctcaggtccttctcaagcagcctcttctcgatggcatccttggcctttttgaaggcctgca |
| | | | | | | gctcttttggggacagagacttgaactgagcaatgtggcaatcctttgcttccactgggagcctg |
| | | | | | | gtggccctggggacagggtcagcttgggttcttgtcagcactgcggccagcagcagaggcaa |
| | | | | | | cagcaggaggagcat |
| 317 | rcCDS_mouse_ | Mouse interferon- | rcCDS | DNA or | 582 | tcagacacactggtctccactggccacacacttgaggtcccggaggagcagttgaaacaggtt |
| | IFN_lambda_ | lambda 3 precursor | | RNA | | ggaggtgacagagtcctccaggcagccaggagtctccttgctctgggcctcctggagcctgtg |
| | 3_precursor | (Mus musculus) | | | | cagccagcgggagaggcggcgactcgggggcttgggctctgctgtggcctgaagctgtgtac |
| | | | | | | aggtctgcagctgggagtgaatgtggctcagtgtatgaagaggctggcccaggatggtggtca |
| | | | | | | gggctgagtcatttatgttctcccagaccttcagggtcagggccacctcagcctgcaaggccttg |
| | | | | | | gggcgctcttggacctgcagctgcttcaggtcccaggccctggagatgaggtgggaactgca |
| | | | | | | cctcatgtccttctcaagcagcctcttctcgatggcacccttggcctttttgaaggcctgcagctct |
| | | | | | | tttggggacagagacttgaactgagcaatgtggcaatcctttgcttccactgggagcctggtggc |
| | | | | | | cctggggacagggtcagcttgggttcttgtcagcactgcggccagcagcagaggcaacagca |
| | | | | | | ggaggagcat |
| 318 | rcCDS_mouse_ | Mouse interferon- | rcCDS | DNA or | 600 | ctatttaagttcttgcttgaaggtgggtgattctgatatcattttgagtagttttctggacttggaaa |
| | IFN_kappa_ | kappa precursor (Mus | | RNA | | atataatgaaacatcttcttatttccactgtgacaatcttccatgcacagaaactgtatttcttattt |
| | precursor | musculus) | | | | atcaggaactttttgattctgaagaaatacttgcccaactccaggtagactgtcaggaagtcctcgga |
| | | | | | | gtgaggatgttccctgttctctttctcctcgtccatgaagcactcttgagcttgccgcacttgtttg |
| | | | | | | aaaagtcccgatctgatacgttcccagtgttcctctgtcactggagggatggagcctttaagattgaa |
| | | | | | | gatagttagtgccagagaggatatacgataggagacggcgtttatctcccttttcatatgctggatg |
| | | | | | | tatggcaaaatctctttaggaaacttaaaatctgtgatatcttttagacatcttaagggaaagccgg |
| | | | | | | tcatggtactgcccagaagtttcacattttccaagatgctatcatccaggtaaacacagttcagag |
| | | | | | | attggatgggcggaatgtatagagccacaagggccaccagccataaaaactttggagtcat |
| 319 | rcCDS_mouse_ | Mouse interleukin-2 | rcCDS | DNA or | 510 | ttattgagggcttgttgagatgatgctttgacagaaggctatccatctcctcagaaagtccaccac |
| | IL2_precursor | precursor (Mus | | RNA | | agttgctgactcatcatcgaattggcactcaaatgtgttgtcagagccctttagttttacaacagtta |
| | | musculus) | | | | ctctgatattgctgatgaaattctcagcatcttccaattgaaagcttttgctttgagtcaaatccaga |
| | | | | | | acatgccgcagaggtccaagttcatcttctaggcactgaagatctttcaattctgtggcctgcttg |
| | | | | | | ggcaagtaaaatttgaaggtgagcatcctggggagtttcaggttcctgtaattctccatcctgctc |
| | | | | | | aggagctcctgtaggtccatcaacagctgctccaggtgctgctgctgctgctgctgctgctgctg |
| | | | | | | ctgctgctgtgcttccgctgtagagcttgaagtggagcttgaagtgggtgcgctgttgacaagga |
| | | | | | | gcacaagtgtcaatgtgacacaggatgcgagctgcatgctgtacat |
| 320 | rcCDS_mouse_ | Mouse interleukin- | rcCDS | DNA or | 1782 | tcaggatcggaccctgcagggaacacatgcccacttgctgcatgaggaattgtaatagcgatcc |
| | IL12A_P2A_ | 12A and interleukin- | | RNA | | tgagcttgcacgcagacattcccgcctttgcattggacttcggtagatgtcttctctacgaggaac |
| | IL12B_ | 12B precursors | | | | gcacctttctggttacacccctcctctgtctccttcatcttttctttcttgcgctggattcgaacaaa |
| | precursors | (bicistronic via P2A | | | | gaacttgagggagaagtaggaatggggagtgctccaggagtcagggtactcccagctgacctc |
| | | sequence) (Mus | | | | cacctgtgagttcttcaaaggcttcatctgcaagttcttggggggtctggtttgatgatgtccctg |
| | | musculus) | | | | atgaagaagctggtgctgtagttctcatatttattctgctgccgtgcttccaacgccagttcaatgg |
| | | | | | | gcagggtctcctcggcagttgggcaggtgacatcctcctggcaggacactgaatacttctcata |
| | | | | | | gtccctttggtccagtgtgaccttctctgcagacagagacgccattccacatgtcactgcccgag |
| | | | | | | agtcaggggaactgctactgctcttgatgttgaacttcaagtccatgtttctttgcaccagccatga |
| | | | | | | gcacgtgaaccgtccggagtaatttggtgcttcacacttcaggaaagtcttgtttttgaaatttttta |
| | | | | | | aaatttcagtggaccaaattccattttccttcttgtggagcagcagatgtgagtggctcagagtctc |
| | | | | | | gcctcctttgtggcaggtgtactggccagcatctagaaactctttgacagtgatggtcagggtctt |
| | | | | | | tccagagcctatgactccatgtctctggtctgaggtccaggtgatgtcatcttcttcaggcgtgtca |
| | | | | | | caggtgaggttcactgtttctccaggggcatcgggagtccagtccacctctacaacataaacgt |
| | | | | | | ctttctccagctcccacatggccatgagtggagacaccagcaaaacgatggcaaaccaggag |
| | | | | | | atggttagcttctgaggacacataggtccagggttctcctccacgtctccagcctgcttcagcag |
| | | | | | | gctgaagttagtagctccgcttccggcggagctcagatagcccatcaccctgttgatggtcacg |
| | | | | | | acgcgggtgctgaaggcgtgaagcaggatgcagagcttcattttcactctgtaagggtctgcttc |
| | | | | | | tcccacaggaggtttctggcgcagagtctcgccattatgattcagagactgcatcagctcatcga |
| | | | | | | tggccaccagcatgcccttgtctagaatgatctgctgatggttgtgattctgaagtgctgcgttgat |
| | | | | | | ggcctggaactctgtctggtacatcttcaagtcctcatagatgctaccaaggcacagggtcatca |
| | | | | | | tcaaagacgtcttctgtgggggcaggcagctccctcttgttgtggaagaagtctctctagtagcc |
| | | | | | | aggcaactctcgttcttgtgtagttccagtggtaaacaggtcttcaatgtgctggtttggtcccgtg |
| | | | | | | tgatgtcttcatgatcgatgtcttcagcagtgcaggaataatgtttcagtttttctctggccgtcttc |
| | | | | | | accatgtcatctgtggtcttcagcaggtttcgggactggctaagacacctggcaggtccagagac |
| | | | | | | tggaatgaccctggccaaactgaggtggtttaggagggcaagggtggccaaaaagaggagg |
| | | | | | | tagcgtgattgacacatgctggaccggcactgagaggagctgctggatgccgagggtgaggc |
| | | | | | | tgttggaacgctgaccat |
| 321 | rcCDS_Syrian_ | Syrian hamster | rcCDS | DNA or | 543 | tcagctctggaagatccaagtgagccttctaatgattaaaaagttccttgagactgctgctcgggt |
| | hamster_IFN_ | interferon-beta 1 | | RNA | | caccatccaggcgcagctgctgtacctcttctctttaaggtaccttcccagctgccagtaatagct |
| | beta_precursor | precursor | | | | tttcaagctaatccttgtagtcaaactttcatcctctggcacttcctttagggtttcattcagaaagac |
| | | (Mesocricetus auratus) | | | | tatctgattgtgaagtttacctaagaaactttcaacgatggtcttattccacccagtgctggagaaa |
| | | | | | | gtgttgttgaagacaagaaagatattctggagcatctcttggatgatgaaggcagtgtctctcttct |
| | | | | | | ccatctgttctgggtggttcacctccataggcatcttgaagaactttctgtctctgaggcagagag |
| | | | | | | gtccacgcagctgtttcaggaggtcctcacactctgaattgcttctgctttgtctggcctcaagttc |
| | | | | | | ctcgtagttgatggacagggatatggggagaaaccacagcagaaatgcagcttgaaggatcc |
| | | | | | | acctgttggtcat |
| 322 | rcCDS_Syrian_ | Syrian hamster | rcCDS | DNA or | 639 | tcagtcacactggtctccactggccacacacttcaggtcccggatgagcaggcggaagaggtt |
| | hamster_IFN_ | interferon-lambda 3 | | RNA | | ggaagtgacagagacttccaggcagccgggggtctccttctcctgggcctcctggagcctgtg |
| | lambda_3_ | precursor | | | | cagccagcgggagaggcggcggctggggggcctgggctctgctgcaggctgaggctgagt |
| | precursor | (Mesocricetus auratus) | | | | acaggcctgcagctgtgagtggatgtggctcagggtgagaagaggctggtccaggatgggg |
| | | | | | | cccaggcccgggtcagtcacgttctccaggaccttcagtgtgagggtcagctcagcctgcaag |
| | | | | | | gcctcggggcgctcctggacctgcagctgcttcaggtcccaggccctggggaagaggcggg |
| | | | | | | agctgcacctgacgtccttcttgagcagccactcttctatggcatccttggccttcttgaaggcct |
| | | | | | | gcagctcttgcgggggcagagacttgaaccgggctatgtgacaatcctttgcatctggtgggag |
| | | | | | | cctggtggccctgggcacaggatgagcgcgggctcccgtcagcaccgcggccagcagcag |
| | | | | | | cagcagcagcagcaggaggagcaggcggcccccagctggcttcatgctgctctctggggtct |
| | | | | | | gtgaggagctgggcat |
| 323 | rcCDS_ferret_ | Domestic ferret | rcCDS | DNA or | 561 | tcagttagggagagaatctgtaagtttatcaaggaagaaaaagctcctgaggatttctgcttgca |
| | IFN_beta_1_ | interferon beta 1 | | RNA | | ctattgtccaggcacagacgctgaactccttcgctttcaggtaccgcacaatccttagataatatct |
| | precursor | precursor (Mustela | | | | cttcaggtgcagaagggtcctgtggtcccaagtgacgttttcctcctgcatgatgtcctccaggat |
| | | putorius furo) | | | | ttcctccagatgttccttctgccagtggagtgtggcaaggaggttctcgacaatgctctcattccat |
| | | | | | | cccgtgcttgagagatttctcctgaaaatatcagaagtcttttggaacatctcaagggtgaccaat |
| | | | | | | acgatgtcctccttctggaacttctgggatttctggatctcctcagggatcttgaagttcatcctgtc |
| | | | | | | cttgaggcagtctttagatgttccattcaactgtagaaggagctcctggcactccacactgctgct |
| | | | | | | gcttagttggaatcgaagtaagttatagttcatggcaagaaccgtggtggagaaatataccagg |
| | | | | | | agagcagtttggaggatacacctgctggtcat |
| 324 | rcCDS_cotton_ | Cotton rat interferon- | rcCDS | DNA or | 573 | ttacttcttctcctcactccatcttgccagcaatttgactgaggaagacagggctctccagacttct |
| | rat_IFN_alpha_ | alpha precursor | | RNA | | gctctgaccacttcccaggcacaggggctgtgtttcctctctttcaggtagacagtgatcctgtgg |
| | precursor | (Sigmodon hispidus) | | | | aagtactgcctcacagtccccagcgagtcttcctcgctcagggtaggttccttcacctcctcctgc |
| | | | | | | tgcatcagacagtcttgtaggtctctgagcttctggtagaggctgtcacagaatgtttccaggag |
| | | | | | | ggttgtctcccaagcagcagatgattcatttgaggtgaaaatgttcaggatctgttggatcacctc |
| | | | | | | acttgagacaaggatggcttgagccttctggagctgctgggcaaccaccttctccagaggggat |
| | | | | | | gcaaagtcctttctgtccttcaggcaggagagagggaagagtgtcctcatttgcaccaggagtg |
| | | | | | | tgaaggctgtgttgttcctgaggttatgaatctgaggcaggtcacatcccagagagcaggttgtc |
| | | | | | | cagtagctcatcaccagcagaaatgtcaggaaagcacatgacctggacat |
| 325 | rcCDS_cotton_ | Cotton rat interferon- | rcCDS | DNA or | 546 | tcagttctggaagatcttaataagtcttcccatgatggaaaagttcctgcggatttctgttcggacta |
| | rat_IFN_beta_ | beta 1 precursor | | RNA | | ctatccaggaacagctgctgtattcatggttttcaagatactcttgcatcctcttataatagctcttca |
| | 1_precursor | (Sigmodon hispidus) | | | | agggtagaatggtggtaaagttccttggagtcaaactttcattctctggtatttcctctagaaagtc |
| | | | | | | catctcgttacggagtttatccaagaagctttcaatgatgtttttattccacccggtgctggaggaat |
| | | | | | | tgctgcggaagatgtggaagatattctggagcatcacttcaatgacggagacagcttcatccttc |
| | | | | | | tctagctgtcttggatgcttcacttccataggaatcttgaagttctgtctatagctgagacagagcg |
| | | | | | | gtccattcatctgattcaagagctctagacattctgaaatgctgctcctttgtctgagctcaagctg |
| | | | | | | cttgtagttgatggagagggctgtagggagaaaccacagcaggaatgcaggttggagaatcc |
| | | | | | | atctgttgaccat |
| 326 | rcCDS_cotton_ | Cotton rat interferon- | rcCDS | DNA or | 603 | ttaggcacactggtctccactggccacacacttcaggtcccgagtcagcaggcggaagaggtt |
| | rat_IFN_ | lambda 2 precursor | | RNA | | gtatgtgacacaggcctccaggctgccgggggtctccttcctctgggcttcctgcagcctttgca |
| | lambda_2_ | (Sigmodon hispidus) | | | | gccagcgagagaggcggcgacttgggggcctgggctctgctgtgagctgaggctgagtaca |
| | precursor | | | | | ggcctgcagctgtgattggatgtggcgcagtgtgcgaaggggctggtccaggatgggatcca |
| | | | | | | gggctgagtcagtcatgttctccaggaccctcagtgtcagggccagctcagccttcagggccat |
| | | | | | | ggggcgctcctggacctgcagctgctgcaggtcccacgccctggggaagaggcgggagctg |
| | | | | | | cacctgatgtcctccttcttgagacgccactcttctatggcatccttggccttcctgaagttctgca |
| | | | | | | gcgcttgcggggacagggacttgaactgggctaggtggcaatgctttgtatctggtggcagcct |
| | | | | | | ggtggccctgggcacagggttagcgcgggttcccgccagcaccggggccagcagcaggag |
| | | | | | | gaggagcccgtggcccccagctctttctggcttcat |
| 327 | rcCDS_BST2_ | BST-2 (Tetherin) | rcCDS | DNA or | 543 | tcactgcagcagagcgctgaggcccagcagcacaatcagcagctggggcgccgcagcgga |
| | Tetherin_CD317 | (CD317) precursor | | RNA | | gctggagtcctgggagctggggtagtacttcttgtccgcgattctcacgcttaagacctggttttct |
| | | (Homo sapiens) | | | | cttctcagtcgctccacctctgcagacgcgtcctgaagcttatggtttaatgtagtgatctctccct |
| | | | | | | caagctcctccactttcttttgtccttgggccttctctgcatccagggaagccattagggccatcac |
| | | | | | | agtgtggttgcaggtggcggcctgggcctccacatcctgaaagcccttctgggcctcggtcag |
| | | | | | | ctcttgttgcaggagatgggtgacattgcgacactccatcactgcccgaaggccgtcccggca |
| | | | | | | ggcctcgctgttggccttgatggtgaagataatcaagggcacccccagaatcacgatgatcag |
| | | | | | | gagcaccagaattcctatccccagcagaagcttacagcgcttatccccgtcttccatgggcactc |
| | | | | | | tgcaatagtcatacgaagtagatgccat |
| 328 | rcCDS_SARS_ | Sudden Acute | rcCDS | DNA or | 336 | ttatgtgtattttaatttgactcctttgagcactggctcagagtcgtcttcatcaaatttgcagcagg |
| | Spike_ | Respiratory Syndrome | | RNA | | atccacaagaacaacagcccttgagacaactacagcaactggtcatacagcaaagcataattgtc |
| | truncation | Coronavirus 2 (SARS- | | | | accattactatggcaatcaagccagctataaaacctagccaaatgtaccatggccattttatatact |
| | | CoV-2) Spike | | | | gctcatactttccaagttcttggagatcgatgagagattcatttaaattcttggcaacctcattgagg |
| | | truncation protein | | | | cggtcaatttctttttgaatgtttacaacactagagactagtggcaataaaacaagaaaaacaaac |
| | | | | | | at |

TABLE 4
Additional Genetic Elements

| SEQ | | | | | |
|---|---|---|---|---|---|
| ID | | | Sequence | | |
| NO: | Name | Description | Type | Length | Sequence |
| 329 | RS_P2A | ribosomal | DNA or | 66 | ggaagcggagctactaacttcagcctgctgaagcaggctggagacgtggaggagaaccctggacct |
| | | skipping sequence | RNA | | |
| 330 | RS_T2A | ribosomal | DNA or | 60 | gagggcagaggaagtctgctaacatgcggtgacgtcgaggagaatcctggcccaactagt |
| | | skipping sequence | RNA | | |
| 331 | Promoter_T7_Core | Core T7 promoter | DNA or | 17 | taatacgactcactata |
| | | | RNA | | |
| 332 | Promoter_T7_AT | initiator_AU | DNA or | 19 | taatacgactcactataat |
| | | | RNA | | |
| 333 | Promoter_T7_AG | initiator_AG | DNA or | 19 | taatacgactcactataag |
| | | | RNA | | |
| 334 | Promoter_T7_GG | initator_GG | DNA or | 19 | taatacgactcactatagg |
| | | | RNA | | |
| 335 | RE_SapI_3p | Sap I site | DNA or | 8 | cgaagagc |
| | | | RNA | | |
| 336 | RE_BbsI_3p | BbsI site | DNA or | 8 | aagtcttc |
| | | | RNA | | |
| 337 | Promoter_CMV_IE2 | IE2 promoter | DNA or | 508 | cgttacataacttacggtaaatggcccgcctggctgaccgcccaacgacccccgcccattgacgtcaata |
| | | | RNA | | atgacgtatgttcccatagtaacgccaatagggactttccattgacgtcaatgggtggagtatttacggtaaa |
| | | | | | ctgcccacttggcagtacatcaagtgtatcatatgccaagtacgccccctattgacgtcaatgacggtaaat |
| | | | | | ggcccgcctggcattatgcccagtacatgaccttatgggactttcctacttggcagtacatctacgtattagt |
| | | | | | catcgctattaccatggtgatgcggttttggcagtacatcaatgggcgtggatagcggtttgactcacggg |
| | | | | | gatttccaagtctccaccccattgacgtcaatgggagtttgttttggcaccaaaatcaacgggactttccaaa |
| | | | | | atgtcgtaacaactccgccccattgacgcaaatgggcggtaggcgtgtacggtgggaggtctatataagc |
| | | | | | agagct |
| 338 | UTR_3p_HDVR_anti | Hepatitis Delta | DNA or | 105 | gggtcggcatggcatctccacctcctcgcggtccgacctgggcatccgaaggaggacgtcgtccactcg |
| | genomic | Virus | RNA | | gatggctaagggagagctcggatcgatccgcctcga |
| | | Antigenomic | | | |
| | | Ribozyme | | | |
| 339 | UTR_3p_BGH_polyA | Bovine Growth | DNA or | 208 | ctgtgccttctagttgccagccatctgttgtttgcccctcccccgtgccttccttgaccctggaaggtgccact |
| | | Hormone | RNA | | cccactgtcctttcctaataaaatgaggaaattgcatcgcattgtctgagtaggtgtcattctattctgggggg |
| | | polyadenylation | | | tggggtggggcaggacagcaagggggaggattgggaagacaatagcaggcatgctgggga |
| | | signal | | | |
| 340 | UTR_5p_HHRz_core | UTR_5p_HHRz_ | DNA or | 39 | ctgatgagtccgtgaggacgaaacccggagtcccgggtc |
| | | core | RNA | | |
| 341 | UTR_5p_IAV_ | UTR_5p_IAV_ | DNA or | 51 | cttgtttctactctgatgagtccgtgaggacgaaacccggagtcccgggtc |
| | antis_HHRz | antis_HHRz | RNA | | |
| 342 | UTR_5p_IBV_ | UTR_5p_IBV_ | DNA or | 51 | cttgttactactctgatgagtccgtgaggacgaaacccggagtcccgggtc |
| | antis_HHRz | antis_HHRz | RNA | | |
| 343 | UTR_5p_SARS2_ | UTR_5p_SARS2_ | DNA or | 51 | taaacctttaatctgatgagtccgtgaggacgaaacccggagtcccgggtc |
| | sense_HHRz | sense_HHRz | RNA | | |
| 344 | UTR_5p_RSV_ | UTR_5p_RSV_ | DNA or | 51 | tttttttctcgtctgatgagtccgtgaggacgaaacccggagtcccgggtc |
| | antis_HHRz | antis_HHRz | RNA | | |
| 345 | UTR_5p_RSV_ | UTR_5p_RSV_ | DNA or | 51 | aatctaattggtctgatgagtccgtgaggacgaaacccggagtcccgggtc |
| | antis_Delta56_ | antis_Delta56_ | RNA | | |
| | HHRz | HHRZ | | | |
| 346 | UTR_5p_RSV_ | UTR_5p_RSV_ | DNA or | 51 | ttttagtgaactctgatgagtccgtgaggacgaaacccggagtcccgggtc |
| | antis_Delta115_ | antis_Delta115_ | RNA | | |
| | HHRz | HHRz | | | |
| 347 | UTR_5p_RSV_ | UTR_5p_RSV_ | DNA or | 51 | attttttcgcgtctgatgagtccgtgaggacgaaacccggagtcccgggtc |
| | sense_HHRz | sense_HHRz | RNA | | |
| 348 | UTR_5p_RSV_ | UTR_5p_RSV_ | DNA or | 51 | cttatttgccccctgatgagtccgtgaggacgaaacccggagtcccgggtc |
| | sense_ | sense_ | RNA | | |
| | DeltaLeader_ | DeltaLeader_ | | | |
| | HHRz | HHRz | | | |
| 349 | UTR_5p_circ_RNA | circular RNA 5′ | DNA or | 257 | gggagaccctcgaccgtcgattgtccactggtcaacaatagatgacttacaactaatcggaaggtgcaga |
| | | | RNA | | gactcgacgggagctaccctaacgtcaagacgagggtaaagagagagtccaattctcaaagccaatag |
| | | | | | gcagtagcgaaagctgcaagagaatgaaaatccgttgaccttaaacggtcgtgtgggttcaagtccctcc |
| | | | | | acccccacgccggaaacgcaatagccgaaaaacaaaaaacaaaaaaacc |
| 350 | UTR_3p_circ_RNA | circular RNA 3′ | DNA or | 188 | cggctattatgcgttaccggcgagacgctacggacttaaataattgagccttaaagaagaaattctttaagt |
| | | | RNA | | ggatgctctcaaactcagggaaacctaaatctagttatagacaaggcaatcctgagccaagccgaagtag |
| | | | | | taattagtaagaccagtggacaatcgacggataacagcatatctag |
| 351 | UTR_5p_generic_ | 5′ UTR for coding | DNA or | 44 | ctcactatttgttttcgcgcccagttgcaaaaagtgtcgccacc |
| | plus_Kozak | region | RNA | | |
| 352 | UTR_3p_generic_ | 3′ UTR for coding | DNA or | 301 | aactgactcgagctggtactgcatgcacgcaatgctagctgcccctttcccgtcctgggtaccccgagtct |
| | Hb | region | RNA | | cccccgacctcgggtcccaggtatgctcccacctccacctgccccactcaccacctctgctagttccaga |
| | | | | | cacctcccaagcacgcagcaatgcagctcaaaacgcttagcctagccacacccccacgggaaacagca |
| | | | | | gtgattaacctttagcaataaacgaaagtttaactaagctatactaaccccagggttggtcaatttcgtgcca |
| | | | | | gccacaccctggagctagc |
| 353 | Promoter_PolI_ | Pol I promoter, | DNA or | 252 | acgggccggcccctgcgtgtggcacgggcggccgggagggcgtccccggcccggcgctgctcccgc |
| | human | human (Homo | RNA | | gtgtgtcctggggttgaccagagggccccgggcgctccgtgtgtggctgcgatggtggcgtttttgggga |
| | | sapiens) | | | caggtgtccgtgtcgcgcgtcgcctgggccggcggcgtggtcggtgacgcgacctcccggccccggg |
| | | | | | ggaggtatatctttcgctccgagtcggcattttgggccgccgggttatt |
| 354 | Promoter_PolI_ | Pol I promoter, | DNA or | 330 | ctggagctttggatctttttttttttttttttttcttttttcctccagaagccctctctgtccctgtcaccgggggcc |
| | mouse | house mouse | RNA | | ctgtacgtctgaggccgagggaaagctatgggcgcggttttctttcattgacctgtcggtcttatcagttctcc |
| | | (Mus musculus) | | | cggttgtcaggtcgaccagttgttccttgaggtccggttcttttcgttatggggtcatttttgggccacctcca |
| | | | | | caggtatgacttcccaggtattctctgtggcctgtcactttcctccctgtctcttttatgcttgtgatcttttctatc |
| | | | | | tgttcctattggacctggagataggt |
| 355 | Terminator_PolI_ | Mouse rRNA | DNA or | 34 | cccccccaacttcggaggtcgaccagtactccgg |
| | mouse | term (classical) | RNA | | |
| 356 | Terminator_PolI_ | Mouse rRNA | DNA or | 29 | ccaacttcggaggtcgaccagtactccgg |
| | mouse_enhanced | term (A5C) | RNA | | |
| 357 | Terminator_PolI_ | Mouse rRNA | DNA or | 30 | cccaacttcggaggtcgaccagtactccgg |
| | mouse_enhanced_d4 | term (A4C) | RNA | | |
| 358 | LV_5p_G03 | LVG03, 5′ end | DNA or | 2200 | ggaagggctaattcactcccaaagaagacaagatatccttgatctgtggatctaccacacacaaggctact |
| | | | RNA | | tccctgattagcagaactacacaccagggccaggggtcagatatccactgacctttggatggtgctacaa |
| | | | | | gctagtaccagttgagccagataaggtagaagaggccaataaaggagagaacaccagcttgttacaccc |
| | | | | | tgtgagcctgcatgggatggatgacccggagagagaagtgttagagtggaggtttgacagccgcctagc |
| | | | | | atttcatcacgtggcccgagagctgcatccggagtacttcaagaactgctgatatcgagcttgctacaagg |
| | | | | | gactttccgctggggactttccagggaggcgtggcctgggcgggactggggagtggcgagccctcaga |
| | | | | | tcctgcatataagcagctgctttttgcctgtactgggtctctctggttagaccagatctgagcctgggagctct |
| | | | | | ctggctaactagggaacccactgcttaagcctcaataaagcttgccttgagtgcttcaagtagtgtgtgccc |
| | | | | | gtctgttgtgtgactctggtaactagagatccctcagacccttttagtcagtgtggaaaatctctagcagtgg |
| | | | | | cgcccgaacagggacttgaaagcgaaagggaaaccagaggagctctctcgacgcaggactcggcttg |
| | | | | | ctgaagcgcgcacggcaagaggcgaggggcggcgactggtgagtacgccaaaaattttgactagcgg |
| | | | | | aggctagaaggagagagatgggtgcgagagcgtcagtattaagcgggggagaattagatcgcgatgg |
| | | | | | gaaaaaattcggttaaggccagggggaaagaaaaaatataaattaaaacatatagtatgggcaagcagg |
| | | | | | gagctagaacgattcgcagttaatcctggcctgttagaaacatcagaaggctgtagacaaatactgggac |
| | | | | | agctacaaccatcccttcagacaggatcagaagaacttagatcattatataatacagtagcaaccctctatt |
| | | | | | gtgtgcatcaaaggatagagataaaagacaccaaggaagctttagacaagatagaggaagagcaaaac |
| | | | | | aaaagtaagaccaccgcacagcaagcggccggccgctgatcttcagacctggaggaggagatatgag |
| | | | | | ggacaattggagaagtgaattatataaatataaagtagtaaaaattgaaccattaggagtagcacccacca |
| | | | | | aggcaaagagaagagtggtgcagagagaaaaaagagcagtgggaataggagctttgttccttgggttct |
| | | | | | tgggagcagcaggaagcactatgggcgcagcgtcaatgacgctgacggtacaggccagacaattattgt |
| | | | | | ctggtatagtgcagcagcagaacaatttgctgagggctattgaggcgcaacagcatctgttgcaactcaca |
| | | | | | gtctggggcatcaagcagctccaggcaagaatcctggctgtggaaagatacctaaaggatcaacagctc |
| | | | | | ctggggatttggggttgctctggaaaactcatttgcaccactgctgtgccttggaatgctagttggagtaata |
| | | | | | aatctctggaacagatttggaatcacacgacctggatggagtgggacagagaaattaacaattacacaag |
| | | | | | cttaatacactccttaattgaagaatcgcaaaaccagcaagaaaagaatgaacaagaattattggaattaga |
| | | | | | taaatgggcaagtttgtggaattggtttaacataacaaattggctgtggtatataaaattattcataatgatagt |
| | | | | | aggaggcttggtaggtttaagaatagtttttgctgtactttctatagtgaatagagttaggcagggatattcac |
| | | | | | cattatcgtttcagacccacctcccaaccccgaggggacccgacaggcccgaaggaatagaagaagaa |
| | | | | | ggtggagagagagacagagacagatccattcgattagtgaacggatctcgacggtatcgcctttaaaaga |
| | | | | | aaaggggggattggggggtacagtgcaggggaaagaatagtagacataatagcaacagacatacaaa |
| | | | | | ctaaagaattacaaaaacaaattacaaaaattcaaaattttcgggtttattacagggacagcagagatccag |
| | | | | | tttggtaccatgcaggtcaattctag |
| 359 | LV_3p_G03 | LVG03, 3′ end | DNA or | 3435 | ggccgcactagggacaggattggtgacagaaaagccccatccttaggcctaagcttgggagttccgcgt |
| | | | RNA | | tacataacttacggtaaatggcccgcctggctgaccgcccaacgacccccgcccattgacgtcaataatg |
| | | | | | acgtatgttcccatagtaacgccaatagggactttccattgacgtcaatgggtggagtatttacggtaaactg |
| | | | | | cccacttggcagtacatcaagtgtatcatatgccaagtacgccccctattgacgtcaatgacggtaaatgg |
| | | | | | cccgcctggcattatgcccagtacatgaccttatgggactttcctacttggcagtacatctacgtattagtcat |
| | | | | | cgctattaccatggtgatgcggttttggcagtacatcaatgggcgtggatagcggtttgactcacggggatt |
| | | | | | tccaagtctccaccccattgacgtcaatgggagtttgttttggcaccaaaatcaacgggactttccaaaatgt |
| | | | | | cgtaacaactccgccccattgacgcaaatgggcggtaggcgtgtacggtgggaggtctatataagcaga |
| | | | | | gctcgtttagtgaaccgtcagatcgcctggagacgccatccacgctgttttgacctccatagaagacaccg |
| | | | | | actctactagaggatctatttccggtgaattcctcgagactagttctagagcggccgcggatcccgcccctc |
| | | | | | tccctcccccccccctaacgttactggccgaagccgcttggaataaggccggtgtgcgtttgtctatatgtt |
| | | | | | attttccaccatattgccgtcttttggcaatgtgagggcccggaaacctggccctgtcttcttgacgagcatt |
| | | | | | cctaggggtctttcccctctcgccaaaggaatgcaaggtctgttgaatgtcgtgaaggaagcagttcctctg |
| | | | | | gaagcttcttgaagacaaacaacgtctgtagcgaccctttgcaggcagcggaaccccccacctggcgac |
| | | | | | aggtgcctctgcggccaaaagccacgtgtataagatacacctgcaaaggcggcacaaccccagtgcca |
| | | | | | cgttgtgagttggatagttgtggaaagagtcaaatggctcacctcaagcgtattcaacaaggggctgaag |
| | | | | | gatgcccagaaggtaccccattgtatgggatctgatctggggcctcggtgcacatgctttacatgtgtttagt |
| | | | | | cgaggttaaaaaacgtctaggccccccgaaccacggggacgtggttttcctttgaaaaacacgatgataa |
| | | | | | tatggtgagcaagggcgaggaggataacatggccatcatcaaggagttcatgcgcttcaaggtgcacat |
| | | | | | ggagggctccgtgaacggccacgagttcgagatcgagggcgagggcgagggccgcccctacgagg |
| | | | | | gcacccagaccgccaagctgaaggtgaccaagggtggccccctgcccttcgcctgggacatcctgtcc |
| | | | | | cctcagttcatgtacggctccaaggcctacgtgaagcaccccgccgacatccccgactacttgaagctgt |
| | | | | | ccttccccgagggcttcaagtgggagcgcgtgatgaacttcgaggacggcggcgtggtgaccgtgacc |
| | | | | | caggactcctccctgcaggacggcgagttcatctacaaggtgaagctgcgcggcaccaacttcccctcc |
| | | | | | gacggccccgtaatgcagaagaagaccatgggctgggaggcctcctccgagcggatgtaccccgagg |
| | | | | | acggcgccctgaagggcgagatcaagcagaggctgaagctgaaggacggcggccactacgacgctg |
| | | | | | aggtcaagaccacctacaaggccaagaagcccgtgcagctgcccggcgcctacaacgtcaacatcaag |
| | | | | | ttggacatcacctcccacaacgaggactacaccatcgtggaacagtacgaacgcgccgagggccgcca |
| | | | | | ctccaccggcggcatggacgagctgtacaagtgaacgcgtctggaacaatcaacctctggattacaaaat |
| | | | | | ttgtgaaagattgactggtattcttaactatgttgctccttttacgctatgtggatacgctgctttaatgcctttgt |
| | | | | | atcatgctattgcttcccgtatggctttcattttctcctccttgtataaatcctggttgctgtctctttatgaggagt |
| | | | | | tgtggcccgttgtcaggcaacgtggcgtggtgtgcactgtgtttgctgacgcaacccccactggttgggg |
| | | | | | cattgccaccacctgtcagctcctttccgggactttcgctttccccctccctattgccacggcggaactcatc |
| | | | | | gccgcctgccttgcccgctgctggacaggggctcggctgttgggcactgacaattccgtggtgttgtcgg |
| | | | | | ggaagctgacgtcctttccatggctgctcgcctgtgttgccacctggattctgcgcgggacgtccttctgct |
| | | | | | acgtcccttcggccctcaatccagcggaccttccttcccgcggcctgctgccggctctgcggcctcttccg |
| | | | | | cgtcttcgccttcgccctcagacgagtcggatctccctttgggccgcctccccgcctggaattaattctgca |
| | | | | | gtcgagacctagaaaaacatggagcaatcacaagtagcaatacagcagctaccaatgctgattgtgcctg |
| | | | | | gctagaagcacaagaggaggaggaggtgggttttccagtcacacctcaggtacctttaagaccaatgact |
| | | | | | tacaaggcagctgtagatcttagccactttttaaaagaaaagaggggactggaagggctaattcactccca |
| | | | | | acgaagacaagatatccttgatctgtggatctaccacacacaaggctacttccctgattagcagaactaca |
| | | | | | caccagggccaggggtcagatatccactgacctttggatggtgctacaagctagtaccagttgagccaga |
| | | | | | taaggtagaagaggccaataaaggagagaacaccagcttgttacaccctgtgagcctgcatgggatgga |
| | | | | | tgacccggagagagaagtgttagagtggaggtttgacagccgcctagcatttcatcacgtggcccgaga |
| | | | | | gctgcatccggagtacttcaagaactgctgatatcgagcttgctacaagggactttccgctggggactttcc |
| | | | | | agggaggcgtggcctggggggactggggagtggcgagccctcagatcctgcatataagcagctgctt |
| | | | | | tttgcctgtactgggtctctctggttagaccagatctgagcctgggagctctctggctaactagggaaccca |
| | | | | | ctgcttaagcctcaataaagcttgccttgagtgcttcaagtagtgtgtgcccgtctgttgtgtgactctggtaa |
| | | | | | ctagagatccctcagacccttttagtcagtgtggaaaatctctagca |
| 360 | LV_5p_G04 | LVG04, 5′ end | DNA or | 4861 | tggaagggctaattcactcccaaagaagacaagatatccttgatctgtggatctaccacacacaaggctac |
| | | | RNA | | ttccctgattagcagaactacacaccagggccaggggtcagatatccactgacctttggatggtgctacaa |
| | | | | | gctagtaccagttgagccagataaggtagaagaggccaataaaggagagaacaccagcttgttacaccc |
| | | | | | tgtgagcctgcatgggatggatgacccggagagagaagtgttagagtggaggtttgacagccgcctagc |
| | | | | | atttcatcacgtggcccgagagctgcatccggagtacttcaagaactgctgatatcgagcttgctacaagg |
| | | | | | gactttccgctggggactttccagggaggcgtggcctggggggactggggagtggcgagccctcaga |
| | | | | | tcctgcatataagcagctgctttttgcctgtactgggtctctctggttagaccagatctgagcctgggagctct |
| | | | | | ctggctaactagggaacccactgcttaagcctcaataaagcttgccttgagtgcttcaagtagtgtgtgccc |
| | | | | | gtctgttgtgtgactctggtaactagagatccctcagacccttttagtcagtgtggaaaatctctagcagtgg |
| | | | | | cgcccgaacagggacttgaaagcgaaagggaaaccagaggagctctctcgacgcaggactcggcttg |
| | | | | | ctgaagcgcgcacggcaagaggcgaggggcggcgactggtgagtacgccaaaaattttgactagcgg |
| | | | | | aggctagaaggagagagatgggtgcgagagcgtcagtattaagcgggggagaattagatcgcgatgg |
| | | | | | gaaaaaattcggttaaggccagggggaaagaaaaaatataaattaaaacatatagtatgggcaagcagg |
| | | | | | gagctagaacgattcgcagttaatcctggcctgttagaaacatcagaaggctgtagacaaatactgggac |
| | | | | | agctacaaccatcccttcagacaggatcagaagaacttagatcattatataatacagtagcaaccctctatt |
| | | | | | gtgtgcatcaaaggatagagataaaagacaccaaggaagctttagacaagatagaggaagagcaaaac |
| | | | | | aaaagtaagaccaccgcacagcaagcggccggccgctgatcttcagacctggaggaggagatatgag |
| | | | | | ggacaattggagaagtgaattatataaatataaagtagtaaaaattgaaccattaggagtagcacccacca |
| | | | | | aggcaaagagaagagtggtgcagagagaaaaaagagcagtgggaataggagctttgttccttgggttct |
| | | | | | tgggagcagcaggaagcactatgggcgcagcgtcaatgacgctgacggtacaggccagacaattattgt |
| | | | | | ctggtatagtgcagcagcagaacaatttgctgagggctattgaggcgcaacagcatctgttgcaactcaca |
| | | | | | gtctggggcatcaagcagctccaggcaagaatcctggctgtggaaagatacctaaaggatcaacagctc |
| | | | | | ctggggatttggggttgctctggaaaactcatttgcaccactgctgtgccttggaatgctagttggagtaata |
| | | | | | aatctctggaacagatttggaatcacacgacctggatggagtgggacagagaaattaacaattacacaag |
| | | | | | cttaatacactccttaattgaagaatcgcaaaaccagcaagaaaagaatgaacaagaattattggaattaga |
| | | | | | taaatgggcaagtttgtggaattggtttaacataacaaattggctgtggtatataaaattattcataatgatagt |
| | | | | | aggaggcttggtaggtttaagaatagtttttgctgtactttctatagtgaatagagttaggcagggatattcac |
| | | | | | cattatcgtttcagacccacctcccaaccccgaggggacccgacaggcccgaaggaatagaagaagaa |
| | | | | | ggtggagagagagacagagacagatccattcgattagtgaacggatctcgacggtatcgcctttaaaaga |
| | | | | | aaaggggggattggggggtacagtgcaggggaaagaatagtagacataatagcaacagacatacaaa |
| | | | | | ctaaagaattacaaaaacaaattacaaaaattcaaaattttcgggtttattacagggacagcagagatccag |
| | | | | | tttatcgataagcttgggagttccgcgttacataacttacggtaaatggcccgcctggctgaccgcccaac |
| | | | | | gacccccgcccattgacgtcaataatgacgtatgttcccatagtaacgccaatagggactttccattgacgt |
| | | | | | caatgggtggagtatttacggtaaactgcccacttggcagtacatcaagtgtatcatatgccaagtacgccc |
| | | | | | cctattgacgtcaatgacggtaaatggcccgcctggcattatgcccagtacatgaccttatgggactttcct |
| | | | | | acttggcagtacatctacgtattagtcatcgctattaccatggtgatgcggttttggcagtacatcaatgggc |
| | | | | | gtggatagcggtttgactcacggggatttccaagtctccaccccattgacgtcaatgggagtttgttttggca |
| | | | | | ccaaaatcaacgggactttccaaaatgtcgtaacaactccgccccattgacgcaaatgggcggtaggcgt |
| | | | | | gtacggtgggaggtctatataagcagagctcgtttagtgaaccgtcagatcgcctggagacgccatccac |
| | | | | | gctgttttgacctccatagaagacaccgactctactagaggatctatttccggtgaattcctcgagactagtt |
| | | | | | ctagagcggccgcggatcccgcccctctccctcccccccccctaacgttactggccgaagccgcttgga |
| | | | | | ataaggccggtgtgcgtttgtctatatgttattttccaccatattgccgtcttttggcaatgtgagggcccgga |
| | | | | | aacctggccctgtcttcttgacgagcattcctaggggtctttcccctctcgccaaaggaatgcaaggtctgtt |
| | | | | | gaatgtcgtgaaggaagcagttcctctggaagcttcttgaagacaaacaacgtctgtagcgaccctttgca |
| | | | | | ggcagcggaaccccccacctggcgacaggtgcctctgcggccaaaagccacgtgtataagatacacct |
| | | | | | gcaaaggcggcacaaccccagtgccacgttgtgagttggatagttgtggaaagagtcaaatggctcacct |
| | | | | | caagcgtattcaacaaggggctgaaggatgcccagaaggtaccccattgtatgggatctgatctggggc |
| | | | | | ctcggtgcacatgctttacatgtgtttagtcgaggttaaaaaacgtctaggccccccgaaccacggggacg |
| | | | | | tggttttcctttgaaaaacacgatgataatatggtgagcaagggcgaggaggataacatggccatcatcaa |
| | | | | | ggagttcatgcgcttcaaggtgcacatggagggctccgtgaacggccacgagttcgagatcgagggcg |
| | | | | | agggcgagggccgcccctacgagggcacccagaccgccaagctgaaggtgaccaagggtggcccc |
| | | | | | ctgcccttcgcctgggacatcctgtcccctcagttcatgtacggctccaaggcctacgtgaagcaccccgc |
| | | | | | cgacatccccgactacttgaagctgtccttccccgagggcttcaagtgggagcgcgtgatgaacttcgag |
| | | | | | gacggcggcgtggtgaccgtgacccaggactcctccctgcaggacggcgagttcatctacaaggtgaa |
| | | | | | gctgcgcggcaccaacttcccctccgacggccccgtaatgcagaagaagaccatgggctgggaggcct |
| | | | | | cctccgagcggatgtaccccgaggacggcgccctgaagggcgagatcaagcagaggctgaagctgaa |
| | | | | | ggacggcggccactacgacgctgaggtcaagaccacctacaaggccaagaagcccgtgcagctgccc |
| | | | | | ggcgcctacaacgtcaacatcaagttggacatcacctcccacaacgaggactacaccatcgtggaacag |
| | | | | | tacgaacgcgccgagggccgccactccaccggcggcatggacgagctgtacaagtgaacgcgtctgg |
| | | | | | aacaatcaacctctggattacaaaatttgtgaaagattgactggtattcttaactatgttgctccttttacgctat |
| | | | | | gtggatacgctgctttaatgcctttgtatcatgctattgcttcccgtatggctttcattttctcctccttgtataaat |
| | | | | | cctggttgctgtctctttatgaggagttgtggcccgttgtcaggcaacgtggcgtggtgtgcactgtgtttgc |
| | | | | | tgacgcaacccccactggttggggcattgccaccacctgtcagctcctttccgggactttcgctttccccct |
| | | | | | ccctattgccacggcggaactcatcgccgcctgccttgcccgctgctggacaggggctcggctgttggg |
| | | | | | cactgacaattccgtggtgttgtcggggaagctgacgtcctttccatggctgctcgcctgtgttgccacctg |
| | | | | | gattctgcgcgggacgtccttctgctacgtcccttcggccctcaatccagcggaccttccttcccgcggcct |
| | | | | | gctgccggctctgcggcctcttccgcgtcttcgccttcgccctcagacgagtcggatctccctttgggccg |
| | | | | | cctccccgcctggaattaattctgcagtcgagacctagaaaaacatggagcaatcacaagtagcaataca |
| | | | | | gcagctaccaatgctgattgtgcctggctagaagcacaagaggaggaggaggtgggttttccagtcaca |
| | | | | | cc |
| 361 | LV_3p_G04 | LVG04, 3′ end | DNA or | 705 | ggtacctttaagaccaatgacttacaaggcagctgtagatcttagccactttttaaaagaaaagaggggact |
| | | | RNA | | ggaagggctaattcactcccaacgaagacaagatatccttgatctgtggatctaccacacacaaggctact |
| | | | | | tccctgattagcagaactacacaccagggccaggggtcagatatccactgacctttggatggtgctacaa |
| | | | | | gctagtaccagttgagccagataaggtagaagaggccaataaaggagagaacaccagcttgttacaccc |
| | | | | | tgtgagcctgcatgggatggatgacccggagagagaagtgttagagtggaggtttgacagccgcctagc |
| | | | | | atttcatcacgtggcccgagagctgcatccggagtacttcaagaactgctgatatcgagcttgctacaagg |
| | | | | | gactttccgctggggactttccagggaggcgtggcctgggcgggactggggagtggcgagccctcaga |
| | | | | | tcctgcatataagcagctgctttttgcctgtactgggtctctctggttagaccagatctgagcctgggagctct |
| | | | | | ctggctaactagggaacccactgcttaagcctcaataaagcttgccttgagtgcttcaagtagtgtgtgccc |
| | | | | | gtctgttgtgtgactctggtaactagagatccctcagacccttttagtcagtgtggaaaatctctagca |
| 362 | LV_5p_G05 | LVG05, 5′ end | DNA or | 4861 | tggaagggctaattcactcccaaagaagacaagatatccttgatctgtggatctaccacacacaaggctac |
| | | | RNA | | ttccctgattagcagaactacacaccagggccaggggtcagatatccactgacctttggatggtgctacaa |
| | | | | | gctagtaccagttgagccagataaggtagaagaggccaataaaggagagaacaccagcttgttacaccc |
| | | | | | tgtgagcctgcatgggatggatgacccggagagagaagtgttagagtggaggtttgacagccgcctagc |
| | | | | | atttcatcacgtggcccgagagctgcatccggagtacttcaagaactgctgatatcgagcttgctacaagg |
| | | | | | gactttccgctggggactttccagggaggcgtggcctgggcgggactggggagtggcgagccctcaga |
| | | | | | tcctgcatataagcagctgctttttgcctgtactgggtctctctggttagaccagatctgagcctgggagctct |
| | | | | | ctggctaactagggaacccactgcttaagcctcaataaagcttgccttgagtgcttcaagtagtgtgtgccc |
| | | | | | gtctgttgtgtgactctggtaactagagatccctcagacccttttagtcagtgtggaaaatctctagcagtgg |
| | | | | | cgcccgaacagggacttgaaagcgaaagggaaaccagaggagctctctcgacgcaggactcggcttg |
| | | | | | ctgaagcgcgcacggcaagaggcgaggggcggcgactggtgagtacgccaaaaattttgactagcgg |
| | | | | | aggctagaaggagagagatgggtgcgagagcgtcagtattaagcgggggagaattagatcgcgatgg |
| | | | | | gaaaaaattcggttaaggccagggggaaagaaaaaatataaattaaaacatatagtatgggcaagcagg |
| | | | | | gagctagaacgattcgcagttaatcctggcctgttagaaacatcagaaggctgtagacaaatactgggac |
| | | | | | agctacaaccatcccttcagacaggatcagaagaacttagatcattatataatacagtagcaaccctctatt |
| | | | | | gtgtgcatcaaaggatagagataaaagacaccaaggaagctttagacaagatagaggaagagcaaaac |
| | | | | | aaaagtaagaccaccgcacagcaagcggccggccgctgatcttcagacctggaggaggagatatgag |
| | | | | | ggacaattggagaagtgaattatataaatataaagtagtaaaaattgaaccattaggagtagcacccacca |
| | | | | | aggcaaagagaagagtggtgcagagagaaaaaagagcagtgggaataggagctttgttccttgggttct |
| | | | | | tgggagcagcaggaagcactatgggcgcagcgtcaatgacgctgacggtacaggccagacaattattgt |
| | | | | | ctggtatagtgcagcagcagaacaatttgctgagggctattgaggcgcaacagcatctgttgcaactcaca |
| | | | | | gtctggggcatcaagcagctccaggcaagaatcctggctgtggaaagatacctaaaggatcaacagctc |
| | | | | | ctggggatttggggttgctctggaaaactcatttgcaccactgctgtgccttggaatgctagttggagtaata |
| | | | | | aatctctggaacagatttggaatcacacgacctggatggagtgggacagagaaattaacaattacacaag |
| | | | | | cttaatacactccttaattgaagaatcgcaaaaccagcaagaaaagaatgaacaagaattattggaattaga |
| | | | | | taaatgggcaagtttgtggaattggtttaacataacaaattggctgtggtatataaaattattcataatgatagt |
| | | | | | aggaggcttggtaggtttaagaatagtttttgctgtactttctatagtgaatagagttaggcagggatattcac |
| | | | | | cattatcgtttcagacccacctcccaaccccgaggggacccgacaggcccgaaggaatagaagaagaa |
| | | | | | ggtggagagagagacagagacagatccattcgattagtgaacggatctcgacggtatcgcctttaaaaga |
| | | | | | aaaggggggattggggggtacagtgcaggggaaagaatagtagacataatagcaacagacatacaaa |
| | | | | | ctaaagaattacaaaaacaaattacaaaaattcaaaattttcgggtttattacagggacagcagagatccag |
| | | | | | tttatcgataagcttgggagttccgcgttacataacttacggtaaatggcccgcctggctgaccgcccaac |
| | | | | | gacccccgcccattgacgtcaataatgacgtatgttcccatagtaacgccaatagggactttccattgacgt |
| | | | | | caatgggtggagtatttacggtaaactgcccacttggcagtacatcaagtgtatcatatgccaagtacgccc |
| | | | | | cctattgacgtcaatgacggtaaatggcccgcctggcattatgcccagtacatgaccttatgggactttcct |
| | | | | | acttggcagtacatctacgtattagtcatcgctattaccatggtgatgcggttttggcagtacatcaatgggc |
| | | | | | gtggatagcggtttgactcacggggatttccaagtctccaccccattgacgtcaatgggagtttgttttggca |
| | | | | | ccaaaatcaacgggactttccaaaatgtcgtaacaactccgccccattgacgcaaatgggcggtaggcgt |
| | | | | | gtacggtgggaggtctatataagcagagctcgtttagtgaaccgtcagatcgcctggagacgccatccac |
| | | | | | gctgttttgacctccatagaagacaccgactctactagaggatctatttccggtgaattcctcgagactagtt |
| | | | | | ctagagcggccgcggatcccgcccctctccctcccccccccctaacgttactggccgaagccgcttgga |
| | | | | | ataaggccggtgtgcgtttgtctatatgttattttccaccatattgccgtcttttggcaatgtgagggcccgga |
| | | | | | aacctggccctgtcttcttgacgagcattcctaggggtctttcccctctcgccaaaggaatgcaaggtctgtt |
| | | | | | gaatgtcgtgaaggaagcagttcctctggaagcttcttgaagacaaacaacgtctgtagcgaccctttgca |
| | | | | | ggcagcggaaccccccacctggcgacaggtgcctctgcggccaaaagccacgtgtataagatacacct |
| | | | | | gcaaaggcggcacaaccccagtgccacgttgtgagttggatagttgtggaaagagtcaaatggctcacct |
| | | | | | caagcgtattcaacaaggggctgaaggatgcccagaaggtaccccattgtatgggatctgatctggggc |
| | | | | | ctcggtgcacatgctttacatgtgtttagtcgaggttaaaaaacgtctaggccccccgaaccacggggacg |
| | | | | | tggttttcctttgaaaaacacgatgataatatggtgagcaagggcgaggaggataacatggccatcatcaa |
| | | | | | ggagttcatgcgcttcaaggtgcacatggagggctccgtgaacggccacgagttcgagatcgagggcg |
| | | | | | agggcgagggccgcccctacgagggcacccagaccgccaagctgaaggtgaccaagggtggcccc |
| | | | | | ctgcccttcgcctgggacatcctgtcccctcagttcatgtacggctccaaggcctacgtgaagcaccccgc |
| | | | | | cgacatccccgactacttgaagctgtccttccccgagggcttcaagtgggagcgcgtgatgaacttcgag |
| | | | | | gacggcggcgtggtgaccgtgacccaggactcctccctgcaggacggcgagttcatctacaaggtgaa |
| | | | | | gctgcgcggcaccaacttcccctccgacggccccgtaatgcagaagaagaccatgggctgggaggcct |
| | | | | | cctccgagcggatgtaccccgaggacggcgccctgaagggcgagatcaagcagaggctgaagctgaa |
| | | | | | ggacggcggccactacgacgctgaggtcaagaccacctacaaggccaagaagcccgtgcagctgccc |
| | | | | | ggcgcctacaacgtcaacatcaagttggacatcacctcccacaacgaggactacaccatcgtggaacag |
| | | | | | tacgaacgcgccgagggccgccactccaccggcggcatggacgagctgtacaagtgaacgcgtctgg |
| | | | | | aacaatcaacctctggattacaaaatttgtgaaagattgactggtattcttaactatgttgctccttttacgctat |
| | | | | | gtggatacgctgctttaatgcctttgtatcatgctattgcttcccgtatggctttcattttctcctccttgtataaat |
| | | | | | cctggttgctgtctctttatgaggagttgtggcccgttgtcaggcaacgtggcgtggtgtgcactgtgtttgc |
| | | | | | tgacgcaacccccactggttggggcattgccaccacctgtcagctcctttccgggactttcgctttccccct |
| | | | | | ccctattgccacggcggaactcatcgccgcctgccttgcccgctgctggacaggggctcggctgttggg |
| | | | | | cactgacaattccgtggtgttgtcggggaagctgacgtcctttccatggctgctcgcctgtgttgccacctg |
| | | | | | gattctgcgcgggacgtccttctgctacgtcccttcggccctcaatccagcggaccttccttcccgcggcct |
| | | | | | gctgccggctctgcggcctcttccgcgtcttcgccttcgccctcagacgagtcggatctccctttgggccg |
| | | | | | cctccccgcctggaattaattctgcagtcgagacctagaaaaacatggagcaatcacaagtagcaataca |
| | | | | | gcagctaccaatgctgattgtgcctggctagaagcacaagaggaggaggaggtgggttttccagtcaca |
| | | | | | cc |
| 363 | LV_3p_G05 | LVG05, 3′ end | DNA or | 1053 | tcgcggccaagggcgaattctgcagatatccatcacactggcggccgcaacttgtttattgcagcttataat |
| | | | RNA | | ggttacaaataaggcaatagcatcacaaatttcacaaataaggcatttttttcactgcattctagttttggtttgt |
| | | | | | ccaaactcatcaatgtatcttatcatgtctggatctcaaatccctcggaagctgcgcctgtcttaggttggagt |
| | | | | | gatacatttttatcacttttacccgtctttggattaggcagtagctctgacggccctcctgtcttaggttagtga |
| | | | | | aaaatgtcactctcttacccgtcattggctgtccagcttagctcgcaggggggtacctttaagaccaatgac |
| | | | | | ttacaaggcagctgtagatcttagccactttttaaaagaaaagaggggactggaagggctaattcactccc |
| | | | | | aacgaagacaagatatccttgatctgtggatctaccacacacaaggctacttccctgattagcagaactac |
| | | | | | acaccagggccaggggtcagatatccactgacctttggatggtgctacaagctagtaccagttgagccag |
| | | | | | ataaggtagaagaggccaataaaggagagaacaccagcttgttacaccctgtgagcctgcatgggatgg |
| | | | | | atgacccggagagagaagtgttagagtggaggtttgacagccgcctagcatttcatcacgtggcccgag |
| | | | | | agctgcatccggagtacttcaagaactgctgatatcgagcttgctacaagggactttccgctggggactttc |
| | | | | | cagggaggcgtggcctgggcgggactggggagtggcgagccctcagatcctgcatataagcagctgct |
| | | | | | ttttgcctgtactgggtctctctggttagaccagatctgagcctgggagctctctggctaactagggaaccc |
| | | | | | actgcttaagcctcaataaagcttgccttgagtgcttcaagtagtgtgtgcccgtctgttgtgtgactctggta |
| | | | | | actagagatccctcagacccttttagtcagtgtggaaaatctctagca |
| 364 | LV_5p_G06 | LVG06, 5′ end | DNA or | 5540 | tggaagggctaattcactcccaaagaagacaagatatccttgatctgtggatctaccacacacaaggctac |
| | | | RNA | | ttccctgattagcagaactacacaccagggccaggggtcagatatccactgacctttggatggtgctacaa |
| | | | | | gctagtaccagttgagccagataaggtagaagaggccaataaaggagagaacaccagcttgttacaccc |
| | | | | | tgtgagcctgcatgggatggatgacccggagagagaagtgttagagtggaggtttgacagccgcctagc |
| | | | | | atttcatcacgtggcccgagagctgcatccggagtacttcaagaactgctgatatcgagcttgctacaagg |
| | | | | | gactttccgctggggactttccagggaggcgtggcctgggcgggactggggagtggcgagccctcaga |
| | | | | | tcctgcatataagcagctgctttttgcctgtactgggtctctctggttagaccagatctgagcctgggagctct |
| | | | | | ctggctaactagggaacccactgcttaagcctcaataaagcttgccttgagtgcttcaagtagtgtgtgccc |
| | | | | | gtctgttgtgtgactctggtaactagagatccctcagacccttttagtcagtgtggaaaatctctagcagtgg |
| | | | | | cgcccgaacagggacttgaaagcgaaagggaaaccagaggagctctctcgacgcaggactcggcttg |
| | | | | | ctgaagcgcgcacggcaagaggcgaggggcggcgactggtgagtacgccaaaaattttgactagcgg |
| | | | | | aggctagaaggagagagatgggtgcgagagcgtcagtattaagcgggggagaattagatcgcgatgg |
| | | | | | gaaaaaattcggttaaggccagggggaaagaaaaaatataaattaaaacatatagtatgggcaagcagg |
| | | | | | gagctagaacgattcgcagttaatcctggcctgttagaaacatcagaaggctgtagacaaatactgggac |
| | | | | | agctacaaccatcccttcagacaggatcagaagaacttagatcattatataatacagtagcaaccctctatt |
| | | | | | gtgtgcatcaaaggatagagataaaagacaccaaggaagctttagacaagatagaggaagagcaaaac |
| | | | | | aaaagtaagaccaccgcacagcaagcggccggccgctgatcttcagacctggaggaggagatatgag |
| | | | | | ggacaattggagaagtgaattatataaatataaagtagtaaaaattgaaccattaggagtagcacccacca |
| | | | | | aggcaaagagaagagtggtgcagagagaaaaaagagcagtgggaataggagctttgttccttgggttct |
| | | | | | tgggagcagcaggaagcactatgggcgcagcgtcaatgacgctgacggtacaggccagacaattattgt |
| | | | | | ctggtatagtgcagcagcagaacaatttgctgagggctattgaggcgcaacagcatctgttgcaactcaca |
| | | | | | gtctggggcatcaagcagctccaggcaagaatcctggctgtggaaagatacctaaaggatcaacagctc |
| | | | | | ctggggatttggggttgctctggaaaactcatttgcaccactgctgtgccttggaatgctagttggagtaata |
| | | | | | aatctctggaacagatttggaatcacacgacctggatggagtgggacagagaaattaacaattacacaag |
| | | | | | cttaatacactccttaattgaagaatcgcaaaaccagcaagaaaagaatgaacaagaattattggaattaga |
| | | | | | taaatgggcaagtttgtggaattggtttaacataacaaattggctgtggtatataaaattattcataatgatagt |
| | | | | | aggaggcttggtaggtttaagaatagtttttgctgtactttctatagtgaatagagttaggcagggatattcac |
| | | | | | cattatcgtttcagacccacctcccaaccccgaggggacccgacaggcccgaaggaatagaagaagaa |
| | | | | | ggtggagagagagacagagacagatccattcgattagtgaacggatctcgacggtatcgcctttaaaaga |
| | | | | | aaaggggggattggggggtacagtgcaggggaaagaatagtagacataatagcaacagacatacaaa |
| | | | | | ctaaagaattacaaaaacaaattacaaaaattcaaaattttcgggtttattacagggacagcagagatccag |
| | | | | | tttatcgataagcttgggagttccgcgttacataacttacggtaaatggcccgcctggctgaccgcccaac |
| | | | | | gacccccgcccattgacgtcaataatgacgtatgttcccatagtaacgccaatagggactttccattgacgt |
| | | | | | caatgggtggagtatttacggtaaactgcccacttggcagtacatcaagtgtatcatatgccaagtacgccc |
| | | | | | cctattgacgtcaatgacggtaaatggcccgcctggcattatgcccagtacatgaccttatgggactttcct |
| | | | | | acttggcagtacatctacgtattagtcatcgctattaccatggtgatgcggttttggcagtacatcaatgggc |
| | | | | | gtggatagcggtttgactcacggggatttccaagtctccaccccattgacgtcaatgggagtttgttttggca |
| | | | | | ccaaaatcaacgggactttccaaaatgtcgtaacaactccgccccattgacgcaaatgggcggtaggcgt |
| | | | | | gtacggtgggaggtctatataagcagagctcgtttagtgaaccgtcagatcgcctggagacgccatccac |
| | | | | | gctgttttgacctccatagaagacaccgactctactagaggatctatttccggtgaattcctcgagactagtt |
| | | | | | ctagagcggccgcggatcccgcccctctccctcccccccccctaacgttactggccgaagccgcttgga |
| | | | | | ataaggccggtgtgcgtttgtctatatgttattttccaccatattgccgtcttttggcaatgtgagggcccgga |
| | | | | | aacctggccctgtcttcttgacgagcattcctaggggtctttcccctctcgccaaaggaatgcaaggtctgtt |
| | | | | | gaatgtcgtgaaggaagcagttcctctggaagcttcttgaagacaaacaacgtctgtagcgaccctttgca |
| | | | | | ggcagcggaaccccccacctggcgacaggtgcctctgcggccaaaagccacgtgtataagatacacct |
| | | | | | gcaaaggcggcacaaccccagtgccacgttgtgagttggatagttgtggaaagagtcaaatggctctcct |
| | | | | | caagcgtattcaacaaggggctgaaggatgcccagaaggtaccccattgtatgggatctgatctggggc |
| | | | | | ctcggtgcacatgctttacatgtgtttagtcgaggttaaaaaaacgtctaggccccccgaaccacggggac |
| | | | | | gtggttttcctttgaaaaacacgatgataatatggccacaaccatgaccgagtacaagcccacggtgcgcc |
| | | | | | tcgccacccgcgacgacgtccccagggccgtacgcaccctcgccgccgcgttcgccgactaccccgc |
| | | | | | cacgcgccacaccgtcgatccggaccgccacatcgagcgggtcaccgagctgcaagaactcttcctca |
| | | | | | cgcgcgtcgggctcgacatcggcaaggtgtgggtcgcggacgacggcgccgcggtggcggtctgga |
| | | | | | ccacgccggagagcgtcgaagcgggggcggtgttcgccgagatcggcccgcgcatggccgagttga |
| | | | | | gcggttcccggctggccgcgcagcaacagatggaaggcctcctggcgccgcaccggcccaaggagc |
| | | | | | ccgcgtggttcctggccaccgtcggcgtctcgcccgaccaccagggcaagggtctgggcagcgccgtc |
| | | | | | gtgctccccggagtggaggcggccgagcgcgccggggtgcccgccttcctggagacctccgcgcccc |
| | | | | | gcaacctccccttctacgagcggctcggcttcaccgtcaccgccgacgtcgaggtgcccgaaggaccg |
| | | | | | cgcacctggtgcatgacccgcaagcccggtgccagtgctggtgggtccgccggcggggagggcaga |
| | | | | | ggaagtctgctaacatgcggtgacgtcgaggagaatcctggcccaactagtgtgagcaagggcgaggc |
| | | | | | agtgatcaaggagttcatgcggttcaaggtgcacatggagggctccatgaacggccacgagttcgagat |
| | | | | | cgagggcgagggcgagggccgcccctacgagggcacccagaccgccaagctgaaggtgaccaagg |
| | | | | | gtggccccctgcccttctcctgggacatcctgtcccctcagttcatgtacggctccagggccttcatcaagc |
| | | | | | accccgccgacatccccgactactataagcagtccttccccgagggcttcaagtgggagcgcgtgatga |
| | | | | | acttcgaggacggcggcgccgtgaccgtgacccaggacacctccctggaggacggcaccctgatctac |
| | | | | | aaggtgaagctccgcggcaccaacttccctcctgacggccccgtaatgcagaagaagacaatgggctg |
| | | | | | ggaagcgtccaccgagcggttgtaccccgaggacggcgtgctgaagggcgacattaagatggccctgc |
| | | | | | gcctgaaggacggcggccgctacctggggacttcaagaccacctacaaggccaagaagcccgtgca |
| | | | | | gatgcccggcgcctacaacgtcgaccgcaagttggacatcacctcccacaacgaggactacaccgtggt |
| | | | | | ggaacagtacgaacgctccgagggccgccactccaccggcggcatggacgagctgtacaagtaaacg |
| | | | | | cgtctggaacaatcaacctctggattacaaaatttgtgaaagattgactggtattcttaactatgttgctcctttt |
| | | | | | acgctatgtggatacgctgctttaatgcctttgtatcatgctattgcttcccgtatggctttcattttctcctcctt |
| | | | | | gtataaatcctggttgctgtctctttatgaggagttgtggcccgttgtcaggcaacgtggcgtggtgtgcactg |
| | | | | | tgtttgctgacgcaacccccactggttggggcattgccaccacctgtcagctcctttccgggactttcgcttt |
| | | | | | ccccctccctattgccacggcggaactcatcgccgcctgccttgcccgctgctggacaggggctcggct |
| | | | | | gttgggcactgacaattccgtggtgttgtcggggaagctgacgtcctttccatggctgctcgcctgtgttgc |
| | | | | | cacctggattctgcgcgggacgtccttctgctacgtcccttcggccctcaatccagcggaccttccttcccg |
| | | | | | cggcctgctgccggctctgcggcctcttccgcgtcttcgccttcgccctcagacgagtcggatctccctttg |
| | | | | | ggccgcctccccgcctggaattaattctgcagtcgagacctagaaaaacatggagcaatcacaagtagc |
| | | | | | aatacagcagctaccaatgctgattgtgcctggctagaagcacaagaggaggaggaggtgggttttcca |
| | | | | | gtcacacc |
| 365 | LV_3p_G06 | LVG06, 3′ end | DNA or | 1053 | tcgcggccaagggcgaattctgcagatatccatcacactggcggccgcaacttgtttattgcagcttataat |
| | | | RNA | | ggttacaaataaggcaatagcatcacaaatttcacaaataaggcatttttttcactgcattctagttttggtttgt |
| | | | | | ccaaactcatcaatgtatcttatcatgtctggatctcaaatccctcggaagctgcgcctgtcttaggttggagt |
| | | | | | gatacatttttatcacttttacccgtctttggattaggcagtagctctgacggccctcctgtcttaggttagtga |
| | | | | | aaaatgtcactctcttacccgtcattggctgtccagcttagctcgcaggggggtacctttaagaccaatgac |
| | | | | | ttacaaggcagctgtagatcttagccactttttaaaagaaaagaggggactggaagggctaattcactccc |
| | | | | | aacgaagacaagatatccttgatctgtggatctaccacacacaaggctacttccctgattagcagaactac |
| | | | | | acaccagggccaggggtcagatatccactgacctttggatggtgctacaagctagtaccagttgagccag |
| | | | | | ataaggtagaagaggccaataaaggagagaacaccagcttgttacaccctgtgagcctgcatgggatgg |
| | | | | | atgacccggagagagaagtgttagagtggaggtttgacagccgcctagcatttcatcacgtggcccgag |
| | | | | | agctgcatccggagtacttcaagaactgctgatatcgagcttgctacaagggactttccgctggggactttc |
| | | | | | cagggaggcgtggcctggggggactggggagtggcgagccctcagatcctgcatataagcagctgct |
| | | | | | ttttgcctgtactgggtctctctggttagaccagatctgagcctgggagctctctggctaactagggaaccc |
| | | | | | actgcttaagcctcaataaagcttgccttgagtgcttcaagtagtgtgtgcccgtctgttgtgtgactctggta |
| | | | | | actagagatccctcagacccttttagtcagtgtggaaaatctctagca |
| 366 | LV_5p_G07 | LVG07, 5′ end | DNA or | 2618 | tggaagggctaattcactcccaaagaagacaagatatccttgatctgtggatctaccacacacaaggctac |
| | | | RNA | | ttccctgattagcagaactacacaccagggccaggggtcagatatccactgacctttggatggtgctacaa |
| | | | | | gctagtaccagttgagccagataaggtagaagaggccaataaaggagagaacaccagcttgttacaccc |
| | | | | | tgtgagcctgcatgggatggatgacccggagagagaagtgttagagtggaggtttgacagccgcctagc |
| | | | | | atttcatcacgtggcccgagagctgcatccggagtacttcaagaactgctgatatcgagcttgctacaagg |
| | | | | | gactttccgctggggactttccagggaggcgtggcctgggcgggactggggagtggcgagccctcaga |
| | | | | | tcctgcatataagcagctgctttttgcctgtactgggtctctctggttagaccagatctgagcctgggagctct |
| | | | | | ctggctaactagggaacccactgcttaagcctcaataaagcttgccttgagtgcttcaagtagtgtgtgccc |
| | | | | | gtctgttgtgtgactctggtaactagagatccctcagacccttttagtcagtgtggaaaatctctagcagtgg |
| | | | | | cgcccgaacagggacttgaaagcgaaagggaaaccagaggagctctctcgacgcaggactcggcttg |
| | | | | | ctgaagcgcgcacggcaagaggcgaggggcggcgactggtgagtacgccaaaaattttgactagcgg |
| | | | | | aggctagaaggagagagatgggtgcgagagcgtcagtattaagcgggggagaattagatcgcgatgg |
| | | | | | gaaaaaattcggttaaggccagggggaaagaaaaaatataaattaaaacatatagtatgggcaagcagg |
| | | | | | gagctagaacgattcgcagttaatcctggcctgttagaaacatcagaaggctgtagacaaatactgggac |
| | | | | | agctacaaccatcccttcagacaggatcagaagaacttagatcattatataatacagtagcaaccctctatt |
| | | | | | gtgtgcatcaaaggatagagataaaagacaccaaggaagctttagacaagatagaggaagagcaaaac |
| | | | | | aaaagtaagaccaccgcacagcaagcggccggccgctgatcttcagacctggaggaggagatatgag |
| | | | | | ggacaattggagaagtgaattatataaatataaagtagtaaaaattgaaccattaggagtagcacccacca |
| | | | | | aggcaaagagaagagtggtgcagagagaaaaaagagcagtgggaataggagctttgttccttgggttct |
| | | | | | tgggagcagcaggaagcactatgggcgcagcgtcaatgacgctgacggtacaggccagacaattattgt |
| | | | | | ctggtatagtgcagcagcagaacaatttgctgagggctattgaggcgcaacagcatctgttgcaactcaca |
| | | | | | gtctggggcatcaagcagctccaggcaagaatcctggctgtggaaagatacctaaaggatcaacagctc |
| | | | | | ctggggatttggggttgctctggaaaactcatttgcaccactgctgtgccttggaatgctagttggagtaata |
| | | | | | aatctctggaacagatttggaatcacacgacctggatggagtgggacagagaaattaacaattacacaag |
| | | | | | cttaatacactccttaattgaagaatcgcaaaaccagcaagaaaagaatgaacaagaattattggaattaga |
| | | | | | taaatgggcaagtttgtggaattggtttaacataacaaattggctgtggtatataaaattattcataatgatagt |
| | | | | | aggaggcttggtaggtttaagaatagtttttgctgtactttctatagtgaatagagttaggcagggatattcac |
| | | | | | cattatcgtttcagacccacctcccaaccccgaggggacccgacaggcccgaaggaatagaagaagaa |
| | | | | | ggtggagagagagacagagacagatccattcgattagtgaacggatctcgacggtatcgcctttaaaaga |
| | | | | | aaaggggggattggggggtacagtgcaggggaaagaatagtagacataatagcaacagacatacaaa |
| | | | | | ctaaagaattacaaaaacaaattacaaaaattcaaaattttcgggtttattacagggacagcagagatccag |
| | | | | | tttatcgacttaacttgtttattgcagcttataatggttacaaataaggcaatagcatcacaaatttcacaaataa |
| | | | | | ggcatttttttcactgcattctagttttggtttgtccaaactcatcaatgtatcttatcatgtctggatctcaaatc |
| | | | | | cctcggaagctgcgcctgtcttaggttggagtgatacatttttatcacttttacccgtctttggattaggcagtag |
| | | | | | ctctgacggccctcctgtcttaggttagtgaaaaatgtcactctcttacccgtcattggctgtccagcttagct |
| | | | | | cgcaggggaggtggtctggatccgccggcaccggttcgaggcggatcgatccgagctctcccttagcc |
| | | | | | atccgagtggacgacgtcctccttcggatgcccaggtcggaccgcgaggaggtggagatgccatgccg |
| | | | | | accc |
| 367 | LV_3p_G07 | LVG07, 3′ end | DNA or | 3881 | acggttcactaaacgagctctgcttatatagacctcccaccgtacacgcctaccgcccatttgcgtcaatgg |
| | | | RNA | | ggcggagttgttacgacattttggaaagtcccgttgattttggtgccaaaacaaactcccattgacgtcaatg |
| | | | | | gggtggagacttggaaatccccgtgagtcaaaccgctatccacgcccattgatgtactgccaaaaccgca |
| | | | | | tcaccatggtaatagcgatgactaatacgtagatgtactgccaagtaggaaagtcccataaggtcatgtact |
| | | | | | gggcataatgccaggcgggccatttaccgtcattgacgtcaatagggggcgtacttggcatatgatacact |
| | | | | | tgatgtactgccaagtgggcagtttaccgtaaatactccacccattgacgtcaatggaaagtccctattggc |
| | | | | | gttactatgggaacatacgtcattattgacgtcaatgggcgggggtcgttgggcggtcagccaggcgggc |
| | | | | | catttaccgtaagttatgtaacgcggaactccatatatgggctatgaactaatgaccccgctcgaggtgata |
| | | | | | attccacggggttggggttgcgccttttccaaggcagccctgggtttgcgcagggacgcggctgctctgg |
| | | | | | gcgtggttccgggaaacgcagcggcgccgaccctgggtctcgcacattcttcacgtccgttcgcagcgt |
| | | | | | cacccggatcttcgccgctacccttgtgggccccccggcgacgcttcctgctccgcccctaagtcgggaa |
| | | | | | ggttccttgcggttcgcggcgtgccggacgtgacaaacggaagccgcacgtctcactagtaccctcgca |
| | | | | | gacggacagcgccagggagcaatggcagcgcgccgaccgcgatgggctgtggccaatagcggctgc |
| | | | | | tcagcagggcgcgccgagagcagcggccgggaaggggcggtgcgggaggcggggtgtggggcgg |
| | | | | | tagtgtgggccctgttcctgcccgcgcggtgttccgcactcactatttgttttcgcgcccagttgcaaaaagt |
| | | | | | gtcgccaccatgaccgagtacaagcccacggtgcgcctcgccacccgcgacgacgtcccccgggccg |
| | | | | | tacgcaccctcgccgccgcgttcgccgactaccccgccacgcgccacaccgtcgacccggaccgcca |
| | | | | | catcgagcgggtcaccgagctgcaagaactcttcctcacgcgcgtcgggctcgacatcggcaaggtgtg |
| | | | | | ggtcgcggacgacggcgccgcggtggcggtctggaccacgccggagagcgtcgaagcgggggcgg |
| | | | | | tgttcgccgagatcggcccgcgcatggccgagttgagcggttcccggctggccgcgcagcaacagatg |
| | | | | | gaaggcctcctggcgccgcaccggcccaaggagcccgcgtggttcctggccaccgtcggcgtctcgc |
| | | | | | ccgaccaccagggcaagggtctgggcagcgccgtcgtgctccccggagtggaggcggccgagcgcg |
| | | | | | ccggggtgcccgccttcctggagacctccgcgccccgcaacctccccttctacgagcggctcggcttca |
| | | | | | ccgtcaccgccgacgtcgaggtgcccgaaggaccgcgcacctggtgcatgacccgcaagcccggtgc |
| | | | | | cagtgctggtgggtccgccggcggggagggcagaggaagtctgctaacatgcggtgacgtcgaggag |
| | | | | | aatcctggcccaactagtgtgagcaagggcgaggcagtgatcaaggagttcatgcggttcaaggtgcac |
| | | | | | atggagggctccatgaacggccacgagttcgagatcgagggcgagggcgagggccgcccctacgag |
| | | | | | ggcacccagaccgccaagctgaaggtgaccaagggtggccccctgcccttctcctgggacatcctgtcc |
| | | | | | cctcagttcatgtacggctccagggccttcatcaagcaccccgccgacatccccgactactataagcagtc |
| | | | | | cttccccgagggcttcaagtgggagcgcgtgatgaacttcgaggacggcggcgccgtgaccgtgaccc |
| | | | | | aggacacctccctggaggacggcaccctgatctacaaggtgaagctccgcggcaccaacttccctcctg |
| | | | | | acggccccgtaatgcagaagaagacaatgggctgggaagcgtccaccgagcggttgtaccccgagga |
| | | | | | cggcgtgctgaagggcgacattaagatggccctgcgcctgaaggacggcggccgctacctggcggac |
| | | | | | ttcaagaccacctacaaggccaagaagcccgtgcagatgcccggcgcctacaacgtcgaccgcaagtt |
| | | | | | ggacatcacctcccacaacgaggactacaccgtggtggaacagtacgaacgctccgagggccgccact |
| | | | | | ccaccggcggcatggacgagctgtacaagtaaacgcgtctggaacaatcaacctctggattacaaaattt |
| | | | | | gtgaaagattgactggtattcttaactatgttgctccttttacgctatgtggatacgctgctttaatgcctttgtat |
| | | | | | catgctattgcttcccgtatggctttcattttctcctccttgtataaatcctggttgctgtctctttatgaggagttg |
| | | | | | tggcccgttgtcaggcaacgtggcgtggtgtgcactgtgtttgctgacgcaacccccactggttggggcat |
| | | | | | tgccaccacctgtcagctcctttccgggactttcgctttccccctccctattgccacggcggaactcatcgc |
| | | | | | cgcctgccttgcccgctgctggacaggggctcggctgttgggcactgacaattccgtggtgttgtcgggg |
| | | | | | aagctgacgtcctttccatggctgctcgcctgtgttgccacctggattctgcgcgggacgtccttctgctac |
| | | | | | gtcccttcggccctcaatccagcggaccttccttcccgcggcctgctgccggctctgcggcctcttccgcg |
| | | | | | tcttcgccttcgccctcagacgagtcggatctccctttgggccgcctccccgcctggaattaattctgcagt |
| | | | | | cgagacctagaaaaacatggagcaatcacaagtagcaatacagcagctaccaatgctgattgtgcctgg |
| | | | | | ctagaagcacaagaggaggaggaggtgggttttccagtcacacctcaggtacctttaagaccaatgactt |
| | | | | | acaaggcagctgtagatcttagccactttttaaaagaaaagaggggactggaagggctaattcactccca |
| | | | | | acgaagacaagatatccttgatctgtggatctaccacacacaaggctacttccctgattagcagaactaca |
| | | | | | caccagggccaggggtcagatatccactgacctttggatggtgctacaagctagtaccagttgagccaga |
| | | | | | taaggtagaagaggccaataaaggagagaacaccagcttgttacaccctgtgagcctgcatgggatgga |
| | | | | | tgacccggagagagaagtgttagagtggaggtttgacagccgcctagcatttcatcacgtggcccgaga |
| | | | | | gctgcatccggagtacttcaagaactgctgatatcgagcttgctacaagggactttccgctggggactttcc |
| | | | | | agggaggcgtggcctgggcgggactggggagtggcgagccctcagatcctgcatataagcagctgctt |
| | | | | | tttgcctgtactgggtctctctggttagaccagatctgagcctgggagctctctggctaactagggaaccca |
| | | | | | ctgcttaagcctcaataaagcttgccttgagtgcttcaagtagtgtgtgcccgtctgttgtgtgactctggtaa |

TABLE 5
Amino Acid Sequences for Exemplary Polypeptides of Interest

| SEQ | | | | | | |
|---|---|---|---|---|---|---|
| ID | | | | | | |
| NO: | Name | Description | Class | Type | Length | Sequence |
| 368 | Protein_ | hsIFNbeta | poly- | Protein | 187 | MTNKCLLQIALLLCFSTTALSMSYNLLGFLQRSSNFQCQKLLWQL |
| | human_ | precursor | peptide | | | NGRLEYCLKDRMNFDIPEEIKQLQQFQKEDAALTIYEMLQNIFAIF |
| | IFN_beta_ | | | | | RQDSSSTGWNETIVENLLANVYHQINHLKTVLEEKLEKEDFTRGK |
| | 1_ | | | | | LMSSLHLKRYYGRILHYLKAKEYSHCAWTIVRVEILRNFYFINRLT |
| | precursor | | | | | GYLRN |
| 369 | Protein_ | hsIFNlambda | poly- | Protein | 200 | MAAAWTVVLVTLVLGLAVAGPVPTSKPTTTGKGCHIGRFKSLSPQ |
| | human_ | 1 precursor | peptide | | | ELASFKKARDALEESLKLKNWSCSSPVFPGNWDLRLLQVRERPVA |
| | IFN_ | | | | | LEAELALTLKVLEAAAGPALEDVLDQPLHTLHHILSQLQACIQPQP |
| | lambda_1_ | | | | | TAGPRPRGRLHHWLHRLQEAPKKESAGCLEASVTFNLFRLLTRDL |
| | precursor | | | | | KYVADGNLCLRTSTHPEST |
| 370 | Protein_ | hsIFNlambda | poly- | Protein | 196 | MTGDCMPVLVLMAAVLTVTGAVPVARLRGALPDARGCHIAQFKS |
| | human_ | 3 precursor | peptide | | | LSPQELQAFKRAKDALEESLLLKDCKCRSRLFPRTWDLRQLQVRE |
| | IFN_ | | | | | RPVALEAELALTLKVLEATADTDPALGDVLDQPLHTLHHILSQLR |
| | lambda_3_ | | | | | ACIQPQPTAGPRTRGRLHHWLHRLQEAPKKESPGCLEASVTFNLF |
| | precursor | | | | | RLLTRDLNCVASGDLCV |
| 371 | Protein_ | Human | poly- | Protein | 196 | MTGDCMPVLVLMAAVLTVTGAVPVARLRGALPDARGCHIAQFKS |
| | human_ | interferon- | peptide | | | LSPQELQAFKRAKDALEESLLLKDCKCRSRLFPRTWDLRQLQVRE |
| | IFN_ | lambda 3 | | | | RPVALEAELALTLKVLEATADTDPALGDVLDQPLHTLHHILSQLR |
| | lambda_3_ | precursor | | | | ACIQPQPTAGPRTRGRLHHWLHRLQEAPKKESPGCLEASVTFNLF |
| | isoform_ | (Homo | | | | RLLTRDLNCVASGDLCV |
| | 2_ | sapiens) | | | | |
| | precursor | | | | | |
| 372 | Protein_ | Human | poly- | Protein | 200 | MKLDMTGDCMPVLVLMAAVLTVTGAVPVARLRGALPDARGCHI |
| | human_ | interferon- | peptide | | | AQFKSLSPQELQAFKRAKDALEESLLLKDCKCRSRLFPRTWDLRQ |
| | IFN_ | lambda 3 | | | | LQVRERPVALEAELALTLKVLEATADTDPALGDVLDQPLHTLHHI |
| | lambda_3_ | isoform 1 | | | | LSQLRACIQPQPTAGPRTRGRLHHWLHRLQEAPKKESPGCLEASV |
| | isoform_ | precursor | | | | TFNLFRLLTRDLNCVASGDLCV |
| | 1_ | (Homo | | | | |
| | precursor | sapiens) | | | | |
| 373 | Protein_ | Human | poly- | Protein | 200 | MKLDMTGDCTPVLVLMAAVLTVTGAVPVARLHGALPDARGCHI |
| | human_ | interferon- | peptide | | | AQFKSLSPQELQAFKRAKDALEESLLLKDCRCHSRLFPRTWDLRQ |
| | IFN_ | lambda 2 | | | | LQVRERPMALEAELALTLKVLEATADTDPALVDVLDQPLHTLHHI |
| | lambda_ | precursor | | | | LSQFRACIQPQPTAGPRTRGRLHHWLYRLQEAPKKESPGCLEASV |
| | 2_ | (Homo | | | | TFNLFRLLTRDLNCVASGDLCV |
| | precursor | sapiens) | | | | |
| 374 | Protein_ | Human | poly- | Protein | 182 | MNNRWILHAAFLLCFSTTALSINYKQLQLQERTNIRKCQELLEQLN |
| | mouse_ | interferon- | peptide | | | GKINLTYRADFKIPMEMTEKMQKSYTAFAIQEMLQNVFLVFRNNF |
| | IFN_ | lambda 3 | | | | SSTGWNETIVVRLLDELHQQTVFLKTVLEEKQEERLTWEMSSTAL |
| | beta_1_ | precursor | | | | HLKSYYWRVQRYLKLMKYNSYAWMVVRAEIFRNFLIIRRLTRNF |
| | precursor | (Homo | | | | QN |
| | | sapiens) | | | | |
| 375 | Protein_ | Mouse | poly- | Protein | 193 | MLLLLLPLLLAAVLTRTQADPVPRATRLPVEAKDCHIAQFKSLSPK |
| | mouse_ | interferon- | peptide | | | ELQAFKKAKDAIEKRLLEKDLRCSSHLFPRAWDLKQLQVQERPKA |
| | IFN_ | lambda 2 | | | | LQAEVALTLKVWENMTDSALATILGQPLHTLSHIHSQLQTCTQLQ |
| | lambda_ | precursor | | | | ATAEPRSPSRRLSRWLHRLQEAQSKETPGCLEASVTSNLFRLLTRD |
| | 2_ | (Mus | | | | LKCVANGDQCV |
| | precursor | musculus) | | | | |
| 376 | Protein_ | Mouse | poly- | Protein | 202 | MKPETAGGHMLLLLLPLLLAAVLTRTQADPVPRATRLPVEAKDC |
| | mouse_ | interferon- | peptide | | | HIAQFKSLSPKELQAFKKAKDAIEKRLLEKDLRCSSHLFPRAWDLK |
| | IFN_ | lambda 2 | | | | QLQVQERPKALQAEVALTLKVWENMTDSALATILGQPLHTLSHIH |
| | lambda_ | isoform X1 | | | | SQLQTCTQLQATAEPRSPSRRLSRWLHRLQEAQSKETPGCLEASVT |
| | 2_ | (Mus | | | | SNLFRLLTRDLKCVANGDQCV |
| | isoform_ | musculus) | | | | |
| | X1 | | | | | |
| 377 | Protein_ | Mouse | poly- | Protein | 193 | MLLLLLPLLLAAVLTRTQADPVPRATRLPVEAKDCHIAQFKSLSPK |
| | mouse_ | interferon- | peptide | | | ELQAFKKAKGAIEKRLLEKDMRCSSHLISRAWDLKQLQVQERPKA |
| | IFN_ | lambda 3 | | | | LQAEVALTLKVWENINDSALTTILGQPLHTLSHIHSQLQTCTQLQA |
| | lambda_ | precursor | | | | TAEPKPPSRRLSRWLHRLQEAQSKETPGCLEDSVTSNLFQLLLRDL |
| | 3_ | (Mus | | | | KCVASGDQCV |
| | precursor | musculus) | | | | |
| 378 | Protein_ | Mouse | poly- | Protein | 248 | MLLLLLPLLLAAVLTRTQADPVPRATRLPVEAKDCHIAQFKSLSPK |
| | mouse_ | interferon- | peptide | | | ELQAFKKAKGAIEKRLLEKDMRCSSHLISRAWDLKQLQVQERPKA |
| | IFN_ | lambda 3 | | | | LQAEVALTLKVWENINDSALTTILGQPLHTLSHIHSQLQTCVSPGS |
| | lambda_ | isoform X1 | | | | LAPPQCSPATWASPVSQQQPHSPHPLSTDTASGHSRAQAPESPPLP |
| | 3_ | (Mus | | | | LAAQAPGGPEQGDSWLPGGLCHLQPVSTAPPGPQVCGQWRPVCL |
| | isoform_ | musculus) | | | | TWNPPASFRNSSLCGCTKTTP |
| | X1 | | | | | |
| 379 | Protein_ | Syrian | poly- | Protein | 180 | MTNRWILQAAFLLWFLPISLSINYEELEARQSRSNSECEDLLKQLR |
| | Syrian- | hamster | peptide | | | GPLCLRDRKFFKMPMEVNHPEQMEKRDTAFIIQEMLQNIFLVENN |
| | hamster_ | interferon- | | | | TFSSTGWNKTIVESFLGKLHNQIVFLNETLKEVPEDESLTTRISLKS |
| | IFN_ | beta 1 | | | | YYWQLGRYLKEKRYSSCAWMVTRAAVSRNFLIIRRLTWIFQS |
| | beta_ | precursor | | | | |
| | precursor | (Mesocricetus | | | | |
| | | auratus) | | | | |
| 380 | Protein_ | Syrian | poly- | Protein | 180 | MTNRWILQAAFLLWFLPISLSINYEELEARQSRSNSECEDLLKQLR |
| | Syrian- | hamster | peptide | | | GPLCLRDRKFFKMPMEVNHPEQMEKRDAAFIIQEMLQNIFLVENN |
| | hamster_ | interferon- | | | | TFSSTGWNKTIVESFLGKLHNQIVFLNETLKEVPEDESLTTRISLKS |
| | IFN_ | beta 1 | | | | YYWQLGRYLKEKRYSSCAWMVTRAAVSRNFLIIRRLTWIFQS |
| | beta_ | precursor | | | | |
| | precursor_ | (Mesocricetus | | | | |
| | alt | auratus) | | | | |
| 381 | Protein_ | Syrian | poly- | Protein | 212 | MPSSSQTPESSMKPAGGRLLLLLLLLLLLAAVLTGARAHPVPRATR |
| | Syrian- | hamster | peptide | | | LPPDAKDCHIARFKSLPPQELQAFKKAKDAIEEWLLKKDVRCSSRL |
| | hamster_ | interferon- | | | | FPRAWDLKQLQVQERPEALQAELTLTVKVLGNVTDPGLGPILDQP |
| | IFN_ | lambda 2 like | | | | LLTLSHIHSQLQACTQPQPAAEPRPPSRRLSRWLHRLQEAQEKETP |
| | lambda_2_ | (Mesocricetus | | | | GCLEVSVTSNLFRLLIRDLKCVASGDQCD |
| | precursor | auratus) | | | | |
| 382 | Protein_ | Syrian | poly- | Protein | 226 | MCKPGLKSREPCSSGDTWDLTMATLFATGSGDHDAGLGHSLSCPS |
| | Syrian- | hamster | peptide | | | FQAPSRLRRGCHISIFQSISQPELDVFKNAPEALEESLPQKKVQCGT |
| | hamster_ | interferon- | | | | RLSPGGWKLAQLPVQERPEALQAELNLTLKVLENVTDPGLGPILD |
| | IFN_ | lambda 2 like | | | | QPLLTLNHIHSQLQACTQPQPAAEPRPPSRRLSRWLHRLQEAQEKE |
| | lambda_2_ | (Mesocricetus | | | | TPGCLEVSVTSNLFRLLIRDLKCVASGNQCDSVSWPGTAYPRL |
| | precursor_ | auratus) | | | | |
| | alt | | | | | |
| 383 | Protein_ | Syrian | poly- | Protein | 212 | MPSSSQTPESSMKPAGGRLLLLLLLLLLLAAVLTGARAHPVPRATR |
| | Syrian- | hamster | peptide | | | LPPDAKDCHIARFKSLPPQELQAFKKAKDAIEEWLLKKDVRCSSRL |
| | hamster_ | interferon- | | | | FPRAWDLKQLQVQERPEALQAELTLTLKVLENVTDPGLGPILDQP |
| | IFN_ | lambda 3 | | | | LLTLSHIHSQLQACTQPQPAAEPRPPSRRLSRWLHRLQEAQEKETP |
| | lambda_3_ | precursor | | | | GCLEVSVTSNLFRLLIRDLKCVASGDQCD |
| | precursor | (Mesocricetus | | | | |
| | | auratus) | | | | |
| 384 | Protein_ | Domestic | poly- | Protein | 191 | MTMAMVWVLLLVTAGLSMATAGPVPTSKPTTTWKGCDIGRFKSL |
| | ferret_ | ferret | peptide | | | SPRELEAFKMAKDALGNSLKNWSCSSRLFPRNRDLRQLQVWERP |
| | IFN_ | interferon | | | | VALEAELTLTLKVLETMADRSQGSILDQPLHTLRHIHSELQACVKA |
| | lambda_1_ | lambda 1 | | | | QPTVGPHPQGRLHHWLHRLHEASKKESQGCLEASVLFNLFRLLKK |
| | precursor | precursor | | | | DLECVASGDLCV |
| | | (Mustela | | | | |
| | | putorius furo) | | | | |
| 385 | Protein_ | Domestic | poly- | Protein | 190 | MATAWVLVLVTLLLGLTGAGSVPTSNPTSTRRGCLMSRFQSLSPS |
| | ferret_ | ferret | peptide | | | ELKAIRKAKDALENALSLKNWSCGSCLFPQDRDLRVLQAWERPV |
| | IFN_ | interferon | | | | ALEAELALTLKVLGAMADSSLGVVLDQPLRLLRHVHSELQACVP |
| | lambda_1_ | lambda 1 like | | | | ALPTAGPRARGLLHHWLHRLQKAPKESQGCLEASVTFNLLRLLT |
| | like_ | precursor | | | | WDLKCVASGDMCV |
| | precursor | (Mustela | | | | |
| | | putorius furo) | | | | |
| 386 | Protein_ | Domestic | poly- | Protein | 192 | MAVGCLPVLIAMVLTRTEAVPGPKPLKVFPDAGGCHLAQFQSLSP |
| | ferret_ | ferret | peptide | | | QELQAFKKAKDTFEESLSLKAWSCRPRLFPRTWDLQQLQVGERPV |
| | IFN_ | interferon | | | | ALEAEVALTLKVLETMADRSLGSILDQPLHTLRHIQSELQACVEAQ |
| | lambda_3_ | lambda 3 | | | | PLAGPRPRGRLHHWLHRLHEAPKKEPLGCLENSVMFNLFRLLTRD |
| | precursor | precursor | | | | LKCVASGDLCA |
| | | (Mustela | | | | |
| | | putorius furo) | | | | |
| 387 | Protein_ | Domestic | poly- | Protein | 186 | MTSRCILQTALLVYFSTTVLAMNYNLLRFQLSSSSVECQELLLQLN |
| | ferret_ | ferret | peptide | | | GTSKDCLKDRMNFKIPEEIQKSQKFQKEDIVLVTLEMFQKTSDIFR |
| | IFN_ | interferon- | | | | RNLSSTGWNESIVENLLATLHWQKEHLEEILEDIMQEENVTWDHR |
| | beta_1_ | beta 1 | | | | TLLHLKRYYLRIVRYLKAKEFSVCAWTIVQAEILRSFFFLDKLTDS |
| | precursor | precursor | | | | LPN |
| 388 | Protein_ | Cotton rat | poly- | Protein | 190 | MSRSCAFLTFLLVMSYWTTCSLGCDLPQIHNLRNNTAFTLLVQMR |
| | cotton_ | interferon- | peptide | | | TLFPLSCLKDRKDFASPLEKVVAQQLQKAQAILVSSEVIQQILNIFT |
| | rat_IFN_ | alpha | | | | SNESSAAWETTLLETFCDSLYQKLRDLQDCLMQQEEVKEPTLSEE |
| | alpha_ | precursor | | | | DSLGTVRQYFHRITVYLKERKHSPCAWEVVRAEVWRALSSSVKL |
| | precursor | (Sigmodon | | | | LARWSEEKK |
| | | hispidus) | | | | |
| 389 | Protein_ | Cotton rat | poly- | Protein | 181 | MVNRWILQPAFLLWFLPTALSINYKQLELRQRSSISECLELLNQMN |
| | cotton_ | interferon- | peptide | | | GPLCLSYRQNFKIPMEVKHPRQLEKDEAVSVIEVMLQNIFHIFRSN |
| | rat_IFN_ | beta 1 | | | | SSSTGWNKNIIESFLDKLRNEMDFLEEIPENESLTPRNFTTILPLKSY |
| | beta_1_ | precursor | | | | YKRMQEYLENHEYSSCSWIVVRTEIRRNFSIMGRLIKIFQN |
| | precursor | (Sigmodon | | | | |
| | | hispidus) | | | | |
| 390 | Protein_ | Cotton rat | poly- | Protein | 200 | MKPERAGGHGLLLLLLAPVLAGTRANPVPRATRLPPDTKHCHLA |
| | cotton_ | interferon- | peptide | | | QFKSLSPQALQNFRKAKDAIEEWRLKKEDIRCSSRLFPRAWDLQQ |
| | rat_IFN_ | lambda 2 | | | | LQVQERPMALKAELALTLRVLENMTDSALDPILDQPLRTLRHIQSQ |
| | lambda_ | precursor | | | | LQACTQPQLTAEPRPPSRRLSRWLQRLQEAQRKETPGSLEACVTY |
| | 2_ | (Sigmodon | | | | NLFRLLTRDLKCVASGDQCA |
| | precursor | hispidus) | | | | |
| 391 | Protein_ | Human | poly- | Protein | 153 | MYRMQLLSCIALSLALVTNSAPTSSSTKKTQLQLEHLLLDLQMILN |
| | human_ | interleukin-2 | peptide | | | GINNYKNPKLTRMLTFKFYMPKKATELKHLQCLEEELKPLEEVLN |
| | IL2_ | precursor | | | | LAQSKNFHLRPRDLISNINVIVLELKGSETTFMCEYADETATIVEFL |
| | precursor | (Homo | | | | NRWITFCQSIISTLT |
| | | sapiens) | | | | |
| 392 | Protein_ | Human | poly- | Protein | 177 | MEICRGLRSHLITLLLFLFHSETICRPSGRKSSKMQAFRIWDVNQKT |
| | human_ | interleukin-1 | peptide | | | FYLRNNQLVAGYLQGPNVNLEEKIDVVPIEPHALFLGIHGGKMCL |
| | IL1RN_ | receptor | | | | SCVKSGDETRLQLEAVNITDLSENRKQDKRFAFIRSDSGPTTSFESA |
| | isoform_ | antagonist | | | | ACPGWFLCTAMEADQPVSLTNMPDEGVMVTKFYFQEDE |
| | 1_ | precursor | | | | |
| | precursor | (Homo | | | | |
| | | sapiens) | | | | |
| 393 | Protein_ | Human tumor | poly- | Protein | 233 | MSTESMIRDVELAEEALPKKTGGPQGSRRCLFLSLFSFLIVAGATT |
| | human_ | necrosis | peptide | | | LFCLLHFGVIGPQREEFPRDLSLISPLAQAVRSSSRTPSDKPVAHVV |
| | TNF_ | factor | | | | ANPQAEGQLQWLNRRANALLANGVELRDNQLVVPSEGLYLIYSQ |
| | precursor | precursor | | | | VLFKGQGCPSTHVLLTHTISRIAVSYQTKVNLLSAIKSPCQRETPEG |
| | | (Homo | | | | AEAKPWYEPIYLGGVFQLEKGDRLSAEINRPDYLDFAESGQVYFGI |
| | | sapiens) | | | | IAL |
| 394 | Protein_ | Human | poly- | Protein | 290 | MIETDSGVDDDMACHKIPVEADFLYAYSTAPGYYSWRNSKDGSW |
| | human_ | activated | peptide | | | FIQSLCAMLKQYADKLEFMHILTRVNRKVATEFESFSFDATFHAK |
| | revCaspase3 | Caspase 3 | | | | KQIPCIVSMLTKELYFYHDEVDGGSPMENTENSVDSKSIKNLEPKII |
| | | (Homo | | | | HGSESMDSGISLDNSYKMDYPEMGLCIIINNKNFHKSTGMTSRSGT |
| | | sapiens) | | | | DVDAANLRETFRNLKYEVRNKNDLTREEIVELMRDVSKEDHSKRS |
| | | | | | | SFVCVLLSHGEEGIIFGTNGPVDLKKITNFFRGDRCRSLTGKPKLFII |
| | | | | | | QACRGTELDCGIETD |
| 395 | Protein_ | BST-2 | poly- | Protein | 153 | MYRMQLLSCIALSLALVTNSAPTSSSTKKTQLQLEHLLLDLQMILN |
| | BST2_ | (Tetherin) | peptide | | | GINNYKNPKLTRMLTFKFYMPKKATELKHLQCLEEELKPLEEVLN |
| | Tetherin_ | (CD317) | | | | LAQSKNFHLRPRDLISNINVIVLELKGSETTFMCEYADETATIVEFL |
| | CD317 | precursor | | | | NRWITFCQSIISTLT |
| | | (Homo | | | | |
| | | sapiens) | | | | |
| 396 | Protein_ | Sudden Acute | poly- | Protein | 111 | MFVFLVLLPLVSSVVNIQKEIDRLNEVAKNLNESLIDLQELGKYEQ |
| | SARS_ | Respiratory | peptide | | | YIKWPWYIWLGFIAGLIAIVMVTIMLCCMTSCCSCLKGCCSCGSCC |
| | Spike_ | Syndrome | | | | KFDEDDSEPVLKGVKLKYT |
| | truncation | Coronavirus | | | | |
| | | 2 (SARS- | | | | |
| | | CoV-1) Spike | | | | |
| | | truncation | | | | |
| | | protein | | | | |
| 397 | Protein_ | Human | poly- | Protein | 240 | MCPARSLLLVATLVLLDHLSLARNLPVATPDPGMFPCLHHSQNLL |
| | human_ | interleukin- | peptide | | | RAVSNMLQKARQTLEFYPCTSEEIDHEDITKDKTSTVEACLPLELT |
| | IL12A_ | 12A | | | | KNESCLNSRETSFITNGSCLASRKTSFMMALCLSSIYEDLKMYQVE |
| | P2A_ | precursor | | | | FKTMNAKLLMDPKRQIFLDQNMLAVIDELMQALNENSETVPQKSS |
| | cleaved | after P2A | | | | LEEPDFYKTKIKLCILLHAFRIRAVTIDRVMSYLNASGSGATNFSLL |
| | | separation | | | | KQAGDVEENPG |
| | | with C- | | | | |
| | | terminal 21 | | | | |
| | | amino acid | | | | |
| | | P2A tail | | | | |
| 398 | Protein_ | Human | poly- | Protein | 329 | PMCHQQLVISWFSLVFLASPLVAIWELKKDVYVVELDWYPDAPG |
| | human_ | interleukin- | peptide | | | EMVVLTCDTPEEDGITWTLDQSSEVLGSGKTLTIQVKEFGDAGQY |
| | P2A_ | 12B | | | | TCHKGGEVLSHSLLLLHKKEDGIWSTDILKDQKEPKNKTFLRCEA |
| | IL12B_ | precursor | | | | KNYSGRFTCWWLTTISTDLTFSVKSSRGSSDPQGVTCGAATLSAE |
| | cleaved | after P2A | | | | RVRGDNKEYEYSVECQEDSACPAAEESLPIEVMVDAVHKLKYEN |
| | | separation | | | | YTSSFFIRDIIKPDPPKNLQLKPLKNSRQVEVSWEYPDTWSTPHSYF |
| | | with N- | | | | SLTFCVQVQGKSKREKKDRVFTDKTSATVICRKNASISVRAQDRY |
| | | terminal | | | | YSSSWSEWASVPCS |
| | | proline added | | | | |
| 399 | Protein_ | Human | poly- | Protein | 349 | MEAHNASAPFNFTLPPNFGKRPTDLALSVILVFMLFFIMLSLGCTM |
| | human_ | NTCP | peptide | | | EFSKIKAHLWKPKGLAIALVAQYGIMPLTAFVLGKVFRLKNIEALA |
| | NTCP | (sodium | | | | ILVCGCSPGGNLSNVFSLAMKGDMNLSIVMTTCSTFCALGMMPLL |
| | | taurocholate | | | | LYIYSRGIYDGDLKDKVPYKGIVISLVLVLIPCTIGIVLKSKRPQYM |
| | | cotransporting | | | | RYVIKGGMIIILLCSVAVTVLSAINVGKSIMFAMTPLLIATSSLMPFI |
| | | polypeptide) | | | | GFLLGYVLSALFCLNGRCRRTVSMETGCQNVQLCSTILNVAFPPE |
| | | (Homo | | | | VIGPLFFFPLLYMIFQLGEGLLLIAIFWCYEKFKTPKDKTKMIYTAA |
| | | sapiens) | | | | TTEETIPGALGNGTYKGEDCSPCTA |
| 400 | Protein_ | Respiratory | poly- | Protein | 391 | MALSKVKLNDTLNKDQLLSSSKYTIQRSTGDSIDTPNYDVQKHIN |
| | RSV_N | Syncytial | peptide | | | KLCGMLLITEDANHKFTGLIGMLYAMSRLGREDTIKILRDAGYHV |
| | | Virus (RSV) | | | | KANGVDVTTHRQDINGKEMKFEVLTLASLTTEIQINIEIESRKSYKK |
| | | Nucleoprotein | | | | MLKEMGEVAPEYRHDSPDCGMIILCIAALVITKLAAGDRSGLTAVI |
| | | (N) | | | | RRANNVLKNEMKRYKGLLPKDIANSFYEVFEKHPHFIDVFVHFGI |
| | | | | | | AQSSTRGGSRVEGIFAGLFMNAYGAGQVMLRWGVLAKSVKNIML |
| | | | | | | GHASVQAEMEQVVEVYEYAQKLGGEAGFYHILNNPKASLLSLTQ |
| | | | | | | FPHFSSVVLGNAAGLGIMGEYRGTPRNQDLYDAAKAYAEQLKEN |
| | | | | | | GVINYSVLDLTAEELEAIKHQLNPKDNDVEL |
| 401 | Protein_ | Respiratory | poly- | Protein | 126 | MEKFAPEFHGEDANNRATKFLESIKGKFTSPKDPKKKDSIISVNSID |
| | RSV_P | Syncytial | peptide | | | IEVTKESPITSNSTIINPTNETDDTAGNKPNYQRKPLVSFKEDPTPSD |
| | | Virus (RSV) | | | | NPFSKLYKETIETFDNNEEESSYSYEEINDQ |
| | | Phosphoprotein | | | | |
| | | (P) | | | | |
| 402 | Protein_ | Sudden Acute | poly- | Protein | 422 | MSDNGPQSNQRSAPRITFGGPTDSTDNNQNGGRNGARPKQRRPQ |
| | SARS1_N | Respiratory | peptide | | | GLPNNTASWFTALTQHGKEELRFPRGQGVPINTNSGPDDQIGYYR |
| | | Syndrome | | | | RATRRVRGGDGKMKELSPRWYFYYLGTGPEASLPYGANKEGIVW |
| | | Coronavirus | | | | VATEGALNTPKDHIGTRNPNNNAATVLQLPQGTTLPKGFYAEGSR |
| | | 1 (SARS- | | | | GGSQASSRSSSRSRGNSRNSTPGSSRGNSPARMASGGGETALALLL |
| | | CoV-1) | | | | LDRLNQLESKVSGKGQQQQGQTVTKKSAAEASKKPRQKRTATKQ |
| | | Nucleocapsid | | | | YNVTQAFGRRGPEQTQGNFGDQDLIRQGTDYKHWPQIAQFAPSAS |
| | | protein (N) | | | | AFFGMSRIGMEVTPSGTWLTYHGAIKLDDKDPQFKDNVILLNKHI |
| | | | | | | DAYKTFPPTEPKKDKKKKTDEAQPLPQRQKKQPTVTLLPAADMD |
| | | | | | | DFSRQLQNSMSGASADSTQA |
| 403 | Protein_ | Sudden Acute | poly- | Protein | 187 | MAPIKGVTFGEDTVWEVQGYKNVRITFELDERVDKVLNEKCSVY |
| | SARS1_ | Respiratory | peptide | | | TVESGTEVTEFACVVAEAVVKTLQPVSDLLTNMGIDLDEWSVATF |
| | NSP3A | Syndrome | | | | YLFDDAGEENFSSRMYCSFYPPDEEEEDDAECEEEEIDETCEHEYG |
| | | Coronavirus | | | | TEDDYQGLPLEFGASAETVRVEEEEEEDWLDDTTEQSEIEPEPEPTP |
| | | 1 (SARS- | | | | EEPVN |
| | | CoV-1) | | | | |
| | | Nonstructural | | | | |
| | | protein 3A | | | | |
| | | domain | | | | |
| | | protein | | | | |
| | | (nsp3a) | | | | |
| 404 | Protein_ | Sudden Acute | poly- | Protein | 419 | MSDNGPQNQRNAPRITFGGPSDSTGSNQNGERSGARSKQRRPQGL |
| | SARS2_N | Respiratory | peptide | | | PNNTASWFTALTQHGKEDLKFPRGQGVPINTNSSPDDQIGYYRRA |
| | | Syndrome | | | | TRRIRGGDGKMKDLSPRWYFYYLGTGPEAGLPYGANKDGIIWVA |
| | | Coronavirus | | | | TEGALNTPKDHIGTRNPANNAAIVLQLPQGTTLPKGFYAEGSRGGS |
| | | 2 (SARS- | | | | QASSRSSSRSRNSSRNSTPGSSRGTSPARMAGNGGDAALALLLLDR |
| | | CoV-1) | | | | LNQLESKMSGKGQQQQGQTVTKKSAAEASKKPRQKRTATKAYN |
| | | Nucleocapsid | | | | VTQAFGRRGPEQTQGNFGDQELIRQGTDYKHWPQIAQFAPSASAF |
| | | protein (N) | | | | FGMSRIGMEVTPSGTWLTYTGAIKLDDKDPNFKDQVILLNKHIDA |
| | | | | | | YKTFPPTEPKKDKKKKADETQALPQRQKKQQTVTLLPAADLDDFS |
| | | | | | | KQLQQSMSSADSTQA |
| 405 | Protein_ | Sudden Acute | poly- | Protein | 207 | MAPTKVTFGDDTVIEVQGYKSVNITFELDERIDKVLNEKCSAYTVE |
| | SARS2_ | Respiratory | peptide | | | LGTEVNEFACVVADAVIKTLQPVSELLTPLGIDLDEWSMATYYLF |
| | NSP3A | Syndrome | | | | DESGEFKLASHMYCSFYPPDEDEEEGDCEEEEFEPSTQYEYGTEDD |
| | | Coronavirus | | | | YQGKPLEFGATSAALQPEEEQEEDWLDDDSQQTVGQQDGSEDNQ |
| | | 2 (SARS- | | | | TTTIQTIVEVQPQLEMELTPVVQTIE |
| | | CoV-2) | | | | |
| | | Nonstructural | | | | |
| | | protein 3A | | | | |
| | | domain | | | | |
| | | protein | | | | |
| | | (nsp3a) | | | | |
| 406 | Protein_ | Unprocessed | poly- | Protein | 19 | ATNFSLLKQAGDVEENPGP |
| | P2A_ | & Translated | peptide | | | |
| | unprocessed | P2A | | | | |
| | | Ribosomal | | | | |
| | | Skipping | | | | |
| | | Sequence | | | | |

TABLE 6
provides a summary of nucleotide positions at which variation
may be allowed for each of the indicated regions.

| | | SEQ | | Some variation |
|---|---|---|---|---|
| Target | L Region Name or | ID | Sequence | allowed at these |
| Virus | R Region Name | NO | Length | positions |
| influenza | antis_5p_IAV | 1 | 45 | 14-37 |
| influenza | antis_5p_IAV_seg01 | 2 | 34 | 14-26 |
| influenza | antis_5p_IAV_seg02 | 3 | 43 | 14-35 |
| influenza | antis_5p_IAV_seg03 | 4 | 58 | 14-50 |
| influenza | antis_5p_IAV_seg04 | 5 | 45 | 14-37 |
| influenza | antis_5p_IAV_seg05 | 6 | 23 | 14-15 |
| influenza | antis_5p_IAV_seg06 | 7 | 28 | 14-20 |
| influenza | antis_5p_IAV_seg07 | 8 | 20 | 14-15 |
| influenza | antis_5p_IAV_seg08 | 9 | 26 | 14-18 |
| influenza | antis_5p_IBV | 10 | 94 | 14-86 |
| influenza | antis_5p_IBV_seg01 | 11 | 89 | 14-81 |
| influenza | antis_5p_IBV_seg02 | 12 | 60 | 14-52 |
| influenza | antis_5p_IBV_seg03 | 13 | 95 | 14-87 |
| influenza | antis_5p_IBV_seg05 | 14 | 101 | 14-93 |
| influenza | antis_5p_IBV_seg06 | 15 | 103 | 14-95 |
| influenza | antis_5p_IBV_seg07 | 16 | 89 | 14-81 |
| influenza | antis_5p_IBV_seg08 | 17 | 30 | 14-22 |
| influenza | antis_3p_IAV | 18 | 32 | 8-20 |
| influenza | antis_3p_IAV_enhanced | 19 | 32 | 8-20 |
| influenza | antis_3p_IAV_seg01 | 20 | 27 | 8-15 |
| influenza | antis_3p_IAV_seg01_enhanced | 21 | 27 | 8-15 |
| influenza | antis_3p_IAV_seg01_m4 | 22 | 27 | 8-15 |
| influenza | antis_3p_IAV_seg01_m4_enhanced | 23 | 27 | 8-15 |
| influenza | antis_3p_IAV_seg02 | 24 | 24 | 8-12 |
| influenza | antis_3p_IAV_seg02_enhanced | 25 | 24 | 8-12 |
| influenza | antis_3p_IAV_seg02_m4 | 26 | 24 | 8-12 |
| influenza | antis_3p_IAV_seg02_m4_enhanced | 27 | 24 | 8-12 |
| influenza | antis_3p_IAV_seg03 | 28 | 24 | 8-12 |
| influenza | antis_3p_IAV_seg03_enhanced | 29 | 24 | 8-12 |
| influenza | antis_3p_IAV_seg03_m4 | 30 | 24 | 8-12 |
| influenza | antis_3p_IAV_seg03_m4_enhanced | 31 | 24 | 8-12 |
| influenza | antis_3p_IAV_seg05 | 32 | 45 | 8-33 |
| influenza | antis_3p_IAV_seg05_enhanced | 33 | 45 | 8-33 |
| influenza | antis_3p_IAV_seg06 | 34 | 20 | 5-8 |
| influenza | antis_3p_IAV_seg06_enhanced | 35 | 20 | 5-8 |
| influenza | antis_3p_IAV_seg06_m4 | 36 | 20 | 5-8 |
| influenza | antis_3p_IAV_seg06_m4_enhanced | 37 | 20 | 5-8 |
| influenza | antis_3p_IAV_seg07 | 38 | 25 | 8-13 |
| influenza | antis_3p_IAV_seg07_enhanced | 39 | 25 | 8-13 |
| influenza | antis_3p_IAV_seg07_m4 | 40 | 25 | 8-13 |
| influenza | antis_3p_IAV_seg07_m4_enhanced | 41 | 25 | 8-13 |
| influenza | antis_3p_IAV_seg08 | 42 | 26 | 8-14 |
| influenza | antis_3p_IAV_seg08_enhanced | 43 | 26 | 8-14 |
| influenza | antis_3p_IBV | 44 | 33 | 8-21 |
| influenza | antis_3p_IBV_enhanced | 45 | 33 | 8-21 |
| influenza | antis_3p_IBV_seg01 | 46 | 21 | 5-9 |
| influenza | antis_3p_IBV_seg01_enhanced | 47 | 21 | 5-9 |
| influenza | antis_3p_IBV_seg02 | 48 | 23 | 8-11 |
| influenza | antis_3p_IBV_seg02_enhanced | 49 | 23 | 8-11 |
| influenza | antis_3p_IBV_seg03 | 50 | 29 | 8-17 |
| influenza | antis_3p_IBV_seg03_enhanced | 51 | 29 | 8-17 |
| influenza | antis_3p_IBV_seg05 | 52 | 60 | 8-48 |
| influenza | antis_3p_IBV_seg05_enhanced | 53 | 60 | 8-48 |
| influenza | antis_3p_IBV_seg06 | 54 | 46 | 8-34 |
| influenza | antis_3p_IBV_seg06_enhanced | 55 | 46 | 8-34 |
| influenza | antis_3p_IBV_seg07 | 56 | 24 | 8-12 |
| influenza | antis_3p_IBV_seg07_enhanced | 57 | 24 | 8-12 |
| influenza | antis_3p_IBV_seg08 | 58 | 44 | 8-32 |
| influenza | antis_3p_IBV_seg08_enhanced | 59 | 44 | 8-32 |
| sarbecovirus | sense_5p_SARS1 | 60 | 1493 | 39-789 or |
| | | | | 1426-1493 |
| sarbecovirus | sense_5p_SARS1GL | 61 | 1493 | 39-789 or |
| | | | | 1426-1493 |
| sarbecovirus | sense_5p_SARS1GL_nsp1_124 | 62 | 1493 | 39-789 or |
| | | | | 1426-1493 |
| sarbecovirus | sense_5p_SARS1GL_nsp1_124_164 | 63 | 1493 | 39-789 or |
| | | | | 1426-1493 |
| sarbecovirus | sense_5p_SARS1GL_nsp1_164 | 64 | 1493 | 39-789 or |
| | | | | 1426-1493 |
| sarbecovirus | sense_5p_SARS1_nsp1_124 | 65 | 1493 | 39-789 or |
| | | | | 1426-1493 |
| sarbecovirus | sense_5p_SARS1_nsp1_124_164 | 66 | 1493 | 39-789 or |
| | | | | 1426-1493 |
| sarbecovirus | sense_5p_SARS1_nsp1_164 | 67 | 1493 | 39-789 or |
| | | | | 1426-1493 |
| sarbecovirus | sense_5p_SARS2 | 68 | 1501 | 39-789 or |
| | | | | 1434-1501 |
| sarbecovirus | sense_5p_SARS2GL | 69 | 1501 | 39-789 or |
| | | | | 1434-1501 |
| sarbecovirus | sense_5p_SARS2GL_ATG_HP15 | 70 | 1513 | 39-789 or |
| | | | | 1446-1513 |
| sarbecovirus | sense_5p_SARS2GL_ATG_HP30 | 71 | 1522 | 39-789 or |
| | | | | 1455-1522 |
| sarbecovirus | sense_5p_SARS2GL_ATG_HP45 | 72 | 1529 | 39-789 or |
| | | | | 1462-1529 |
| sarbecovirus | sense_5p_SARS2GL_ATG_HP60 | 73 | 1536 | 39-789 or |
| | | | | 1469-1536 |
| sarbecovirus | sense_5p_SARS2GL_GGC_HHRz | 74 | 1552 | 39-789 or |
| | | | | 1485-1552 |
| sarbecovirus | sense_5p_SARS2GL_N250 | 75 | 1753 | 39-789 or |
| | | | | 1686-1753 |
| sarbecovirus | sense_5p_SARS2GL_N250_HP10 | 76 | 1771 | 39-789 or |
| | | | | 1704-1771 |
| sarbecovirus | sense_5p_SARS2GL_N250_HP30 | 77 | 1787 | 39-789 or |
| | | | | 1720-1787 |
| sarbecovirus | sense_5p_SARS2GL_N250_HP50 | 78 | 1801 | 39-789 or |
| | | | | 1734-1801 |
| sarbecovirus | sense_5p_SARS2GL_N250_stop | 79 | 1754 | 39-789 or |
| | | | | 1687-1754 |
| sarbecovirus | sense_5p_SARS2GL_N250_us_ATG | 80 | 1762 | 39-789 or |
| | | | | 1695-1762 |
| sarbecovirus | sense_5p_SARS2GL_PolII_ΔSA_ΔSD | 81 | 1501 | 39-789 or |
| | | | | 1434-1501 |
| sarbecovirus | sense 5p_SARS2GL_hp_ATG2 | 82 | 1510 | 39-789 or |
| | | | | 1443-1510 |
| sarbecovirus | sense_5p_SARS2GL_hp_ATG3 | 83 | 1526 | 39-789 or |
| | | | | 1459-1526 |
| sarbecovirus | sense_5p_SARS2GL_nonactivatable | 84 | 1495 | 39-789 or |
| | | | | 1428-1495 |
| sarbecovirus | sense_5p_SARS2GL_nsp1_124 | 85 | 1501 | 39-789 or |
| | | | | 1434-1501 |
| sarbecovirus | sense_5p_SARS2GL_nsp1_124_164 | 86 | 1501 | 39-789 or |
| | | | | 1434-1501 |
| sarbecovirus | sense_5p_SARS2GL_nsp1_164 | 87 | 1502 | 39-789 or |
| | | | | 1435-1502 |
| sarbecovirus | sense_5p_SARS2GL_uATG_NDegron1 | 88 | 1530 | 39-789 or |
| | | | | 1463-1530 |
| sarbecovirus | sense_5p_SARS2GL_uATG_NDegron2 | 89 | 1533 | 39-789 or |
| | | | | 1466-1533 |
| sarbecovirus | sense_5p_SARS2GL_uATG_overlap | 90 | 1492 | 39-789 or |
| | | | | 1425-1492 |
| sarbecovirus | sense_5p_SARS2GL_Δ300_locC | 93 | 1201 | 39-789 or |
| | | | | 1134-1201 |
| sarbecovirus | sense_5p_SARS2GL_Δ300_locD | 94 | 1231 | 39-789 or |
| | | | | 1164-1231 |
| sarbecovirus | sense_5p_SARS2GL_Δ30_loc02 | 96 | 1471 | 39-769 or |
| | | | | 1471-1471 |
| sarbecovirus | sense_5p_SARS2GL_Δ30_loc03 | 97 | 1471 | 39-789 or |
| | | | | 1404-1471 |
| sarbecovirus | sense_5p_SARS2GL_Δ30_loc04 | 98 | 1471 | 39-789 or |
| | | | | 1404-1471 |
| sarbecovirus | sense_5p_SARS2GL_Δ30_loc05 | 99 | 1471 | 39-789 or |
| | | | | 1404-1471 |
| sarbecovirus | sense_5p_SARS2GL_Δ30_loc06 | 100 | 1471 | 39-789 or |
| | | | | 1404-1471 |
| sarbecovirus | sense_5p_SARS2GL_Δ30_loc07 | 101 | 1471 | 39-789 or |
| | | | | 1404-1471 |
| sarbecovirus | sense_5p_SARS2GL_Δ30_loc08 | 102 | 1471 | 39-789 or |
| | | | | 1404-1471 |
| sarbecovirus | sense_5p_SARS2GL_Δ30_loc09 | 103 | 1471 | 39-789 or |
| | | | | 1404-1471 |
| sarbecovirus | sense_5p_SARS2_ATG_HP15 | 104 | 1513 | 39-789 or |
| | | | | 1446-1513 |
| sarbecovirus | sense_5p_SARS2_ATG_HP30 | 105 | 1522 | 39-789 or |
| | | | | 1455-1522 |
| sarbecovirus | sense_5p_SARS2_ATG_HP45 | 106 | 1529 | 39-789 or |
| | | | | 1462-1529 |
| sarbecovirus | sense_5p_SARS2_ATG_HP60 | 107 | 1536 | 39-789 or |
| | | | | 1469-1536 |
| sarbecovirus | sense_5p_SARS2_GGC_HHRz | 108 | 1552 | 89-839 or |
| | | | | 1485-1552 |
| sarbecovirus | sense_5p_SARS2_N250 | 109 | 1753 | 40-789 or |
| | | | | 1686-1753 |
| sarbecovirus | sense_5p_SARS2_N250_HP10 | 110 | 1771 | 40-789 or |
| | | | | 1704-1771 |
| sarbecovirus | sense_5p_SARS2_N250_HP30 | 111 | 1787 | 40-789 or |
| | | | | 1720-1787 |
| sarbecovirus | sense_5p_SARS2_N250_HP50 | 112 | 1801 | 40-789 or |
| | | | | 1734-1801 |
| sarbecovirus | sense_5p_SARS2_N250_stop | 113 | 1754 | 40-789 or |
| | | | | 1687-1754 |
| sarbecovirus | sense_5p_SARS2_N250_us_ATG | 114 | 1762 | 40-789 or |
| | | | | 1695-1762 |
| sarbecovirus | sense_5p_SARS2_PolII_ΔSA_ΔSD | 115 | 1501 | 40-789 or |
| | | | | 1434-1501 |
| sarbecovirus | sense_5p_SARS2_UTRuATGv1 | 116 | 1501 | 40-789 or |
| | | | | 1434-1501 |
| sarbecovirus | sense_5p_SARS2_UTRuATGv2 | 117 | 1501 | 40-789 or |
| | | | | 1434-1501 |
| sarbecovirus | sense_5p_SARS2_UTRuATGv3 | 118 | 1501 | 40-789 or |
| | | | | 1434-1501 |
| sarbecovirus | sense_5p_SARS2_hp_ATG2 | 119 | 1510 | 40-789 or |
| | | | | 1443-1510 |
| sarbecovirus | sense_5p_SARS2_hp_ATG3 | 120 | 1526 | 40-789 or |
| | | | | 1459-1526 |
| sarbecovirus | sense_5p_SARS2_nsp1_124 | 122 | 1501 | 40-789 or |
| | | | | 1434-1501 |
| sarbecovirus | sense_5p_SARS2_nsp1_124_164 | 123 | 1501 | 40-789 or |
| | | | | 1434-1501 |
| sarbecovirus | sense_5p_SARS2_nsp1_164 | 124 | 1501 | 40-789 or |
| | | | | 1434-1501 |
| sarbecovirus | sense_5p_SARS2_uATG_NDegron1 | 125 | 1530 | 40-789 or |
| | | | | 1463-1530 |
| sarbecovirus | sense_5p_SARS2_uATG_NDegron2 | 126 | 1533 | 40-789 or |
| | | | | 1466-1533 |
| sarbecovirus | sense_5p_SARS2_uATG_overlap | 127 | 1492 | 40-789 or |
| | | | | 1425-1492 |
| sarbecovirus | antis_3p_SARS1_short | 128 | 80 | 20-30 |
| sarbecovirus | sense_3p_SARS1 | 129 | 370 | 20-320 |
| sarbecovirus | sense_3p_SARS2 | 130 | 370 | 20-320 |
| sarbecovirus | sense_3p_SARS2GL_Δ30_loc10 | 131 | 340 | 20-290 |
| sarbecovirus | sense_3p_SARS2GL_Δ30_loc11 | 132 | 340 | 20-290 |
| sarbecovirus | sense_3p_SARS2GL_Δ30_loc12 | 133 | 340 | 20-290 |
| sarbecovirus | sense_3p_SARS2GL_Δ30_loc13 | 134 | 340 | 20-290 |
| sarbecovirus | sense_3p_SARS2GL_Δ30_loc14 | 135 | 340 | 20-290 |
| sarbecovirus | antis_3p_SARS2_short | 136 | 83 | 20-33 |
| sarbecovirus | antis_5p_SARS1 | 137 | 1607 | 40-1557 |
| sarbecovirus | antis_5p_SARS2 | 138 | 352 | 50-312 |
| sarbecovirus | antis_5p_SARS2GL_B | 139 | 1607 | 50-1567 |
| sarbecovirus | antis_5p_SARS2GL_invert_pA | 140 | 1528 | 50-1488 |
| sarbecovirus | antis_5p_SARS2GL_invert_pA_HDVR | 141 | 1633 | 50-1593 |
| sarbecovirus | antis_5p_SARS2_B | 142 | 1610 | 50-1570 |
| sarbecovirus | antis_5p_SARS2_invert_pA | 143 | 1528 | 50-1488 |
| sarbecovirus | antis_5p_SARS2_invert_pA_HDVR | 144 | 1633 | 50-1593 |
| sarbecovirus | antis_3p_SARS2 | 145 | 1501 | 50-1461 |
| sarbecovirus | antis_3p_SARS2GL | 146 | 1501 | 60-1441 |
| sarbecovirus | antis_3p_SARS2_invert_IRES | 147 | 957 | 60-897 |
| RSV | sense_5p_RSV | 148 | 98 | 15-78 |
| RSV | sense_5p_RSV_short | 149 | 53 | 15-33 |
| RSV | sense_5p_RSV_short_3610 | 150 | 55 | 15-35 |
| RSV | sense_5p_RSV_short_add_agg | 151 | 56 | 18-36 |
| RSV | sense_5p_RSV_short_agg_3610 | 152 | 58 | 15-38 |
| RSV | sense_5p_RSV_Δleader | 153 | 54 | 15-34 |
| RSV | sense_3p_RSV | 154 | 227 | 15-207 |
| RSV | sense_3p_RSV_short | 155 | 52 | 15-32 |
| RSV | sense_3p_RSV_Δ115 | 156 | 112 | 15-92 |
| RSV | sense_3p_RSV_Δ56 | 157 | 171 | 15-151 |
| RSV | antis_5p_RSV | 158 | 227 | 15-207 |
| RSV | antis_5p_RSV_add_021nt | 159 | 248 | 15-228 |
| RSV | antis_5p_RSV_add_051nt | 160 | 278 | 15-258 |
| RSV | antis_5p_RSV_add_101nt | 161 | 328 | 15-308 |
| RSV | antis_5p_RSV_add_234nt | 162 | 461 | 15-441 |
| RSV | antis_5p_RSV_add_agg | 163 | 230 | 18-210 |
| RSV | antis_5p_RSV_ag | 164 | 227 | 15-207 |
| RSV | antis_5p_RSV_no_flank | 165 | 167 | 15-147 |
| RSV | antis_5p_RSV_short | 166 | 52 | 15-32 |
| RSV | antis_5p_RSV_short_add_agg | 419 | 55 | 18-35 |
| RSV | antis_5p_RSV_Δ115 | 167 | 112 | 15-92 |
| RSV | antis_5p_RSV_Δ56 | 168 | 171 | 15-151 |
| RSV | antis_3p_RSV | 169 | 98 | 15-78 |
| RSV | antis_3p_RSV_36TrC | 170 | 100 | 15-80 |
| RSV | antis_3p_RSV_36TrC_add_021nt | 171 | 121 | 15-101 |
| RSV | antis_3p_RSV_36TrC_add_051nt | 172 | 151 | 15-131 |
| RSV | antis_3p_RSV_36TrC_add_101nt | 173 | 201 | 15-181 |
| RSV | antis_3p_RSV_36TrC_add_274nt | 174 | 374 | 15-354 |
| RSV | antis_3p_RSV_36TrC_ag | 175 | 100 | 15-80 |
| RSV | antis_3p_RSV_36TrC_no_flank | 176 | 56 | 15-36 |
| RSV | antis_3p_RSV_short | 177 | 53 | 15-33 |
| RSV | antis_3p_RSV_Δleader | 178 | 54 | 15-34 |
| RSV | antis_3p_RSV_short_36TrC | 420 | 55 | 15-35 |
| HPIV1 | sense_3p_HPIV1_FR2011 | 179 | 119 | 17-98 |
| HPIV1 | sense_5p_HPIV1_FR2011 | 180 | 157 | 17-136 |
| HPIV1 | antis_5p_HPIV1_FR2011 | 181 | 157 | 17-136 |
| HPIV1 | antis_5p_HPIV1_FR2011_add_agg | 182 | 163 | 17-142 |
| HPIV1 | antis_5p_HPIV1_FR2011_t28g | 183 | 157 | 17-136 |
| HPIV1 | antis_3p_HPIV1_FR2011 | 184 | 119 | 17-98 |
| HPIV3 | sense_3p_HPIV3_Wash | 185 | 110 | 21-93 |
| HPIV3 | sense_5p_HPIV3_Wash | 186 | 112 | 21-95 |
| HPIV3 | antis_5p_HPIV3_Wash | 187 | 112 | 21-95 |
| HPIV3 | antis_5p_HPIV3_Wash_add_agg | 188 | 118 | 21-101 |
| HPIV3 | antis_5p_HPIV3_Wash_t28g | 189 | 112 | 21-95 |
| HPIV3 | antis_3p_HPIV3_Wash | 190 | 110 | 21-93 |
| HMPV | sense_3p_AMPV | 191 | 53 | 17-32 |
| HMPV | sense_3p_HMPV_NL | 192 | 53 | 17-32 |
| HMPV | sense_5p_AMPV | 193 | 241 | 17-220 |
| HMPV | sense_5p_HMPV_NL | 194 | 241 | 17-220 |
| HMPV | antis_5p_AMPV | 195 | 241 | 21-224 |
| HMPV | antis_5p_HMPV_NL | 196 | 241 | 21-224 |
| HMPV | antis_5p_HMPV_NL_add_agg | 197 | 247 | 21-230 |
| HMPV | antis_5p_HMPV_NL_s2 | 198 | 151 | 21-134 |
| HMPV | antis_5p_HMPV_NL_s2_add_agg | 199 | 157 | 21-140 |
| HMPV | antis_3p_AMPV | 200 | 53 | 17-32 |
| HMPV | antis_3p_HMPV_NL | 201 | 53 | 17-32 |
| NiV | sense_3p_NiV_UMMC1 | 202 | 112 | 17-91 |
| NiV | sense_5p_NiV_UMMC1 | 203 | 98 | 17-77 |
| NiV | antis_5p_NiV_UMMC1 | 204 | 98 | 17-77 |
| NiV | antis_5p_NiV_UMMC1_add_agg | 205 | 104 | 17-83 |
| NiV | antis_3p_NiV_UMMC1 | 206 | 112 | 17-91 |
| HeV | sense_5p_HeV_AUS | 207 | 98 | 17-77 |
| HeV | sense_3p_HeV_AUS | 208 | 112 | 17-91 |
| HeV | antis_5p_HeV_AUS | 209 | 98 | 17-77 |
| HeV | antis_5p_HeV_AUS_add_agg | 210 | 104 | 17-83 |
| HeV | antis_3p_HeV_AUS | 211 | 112 | 17-91 |
| HBV | sense_5p_HBV_21 | 212 | 1626 | 101-1326 |
| HBV | sense_5p_HBV_31 | 213 | 1591 | 101-1291 |
| HBV | sense_5p_HBV_41 | 214 | 1625 | 101-1325 |
| HBV | sense_5p_HBV_60 | 215 | 315 | 101-15 |
| HBV | sense_5p_HBV_61 | 216 | 309 | 101-211 |
| HBV | sense_3p_HBV_21 | 217 | 1054 | 101-754 |
| HBV | sense_3p_HBV_31 | 218 | 1090 | 101-790 |
| HBV | sense_3p_HBV_41 | 219 | 1192 | 101-892 |
| HBV | sense_3p_HBV_60 | 220 | 2609 | 101-2309 |
| HBV | antis_3p_HBV_51 | 221 | 2724 | 101-2424 |
| HBV | antis_5p_HBV_01 | 222 | 939 | 101-639 |
| HBV | antis_5p_HBV_02 | 223 | 243 | 101-186 |
| HBV | antis_5p_HBV_50 | 224 | 239 | 101-178 |
| HBV | antis_3p_HBV_01 | 225 | 1323 | 101-1023 |
| HBV | antis_3p_HBV_50 | 226 | 2730 | 101-2430 |
| ZEBOV | antis_5p_ZEBOV | 227 | 740 | 20-710 |
| ZEBOV | antis_5p_ZEBOV_add_agg | 228 | 743 | 23-713 |
| ZEBOV | antis_5p_ZEBOV_Δ3 | 229 | 737 | 17-707 |
| ZEBOV | antis_5p_ZEBOV_Δ3_ag | 230 | 737 | 17-707 |
| ZEBOV | antis_3p_ZEBOV | 231 | 469 | 15-449 |
| SEBOV | antis_5p_SEBOV | 232 | 708 | 20-678 |
| SEBOV | antis_5p_SEBOV_add_agg | 233 | 711 | 23-681 |
| SEBOV | antis_5p_SEBOV_Δ3 | 234 | 705 | 23-678 |
| SEBOV | antis_5p_SEBOV_Δ3_ag | 235 | 705 | 23-678 |
| SEBOV | antis_3p_SEBOV | 236 | 457 | 15-437 |
| MARV | antis_5p_MARV | 237 | 635 | 20-605 |
| MARV | antis_5p_MARV_add_a | 238 | 636 | 21-606 |
| MARV | antis_5p_MARV_g1a | 239 | 635 | 20-605 |
| MARV | antis_3p_MARV | 240 | 103 | 15-83 |
| RV | antis_5p_RV | 241 | 49 | 20-34 |
| RV | antis_3p_RV | 242 | 613 | 100-593 |
| EVD68 | antis_5p_EVD68 | 243 | 65 | 30-45 |
| EVD68 | antis_3p_EVD68 | 244 | 697 | 100-677 |
| NDV | antis_5p_NDV | 245 | 191 | 29-171 |
| NDV | antis_5p_NDV_Δ3_add_agg | 246 | 191 | 30-171 |
| NDV | antis_5p_NDV_ag | 247 | 191 | 29-171 |
| NDV | antis_3p_NDV | 248 | 121 | 20-91 |
| EEEV | antis_5p_EEEV | 249 | 374 | 60-274 |
| EEEV | antis_3p_EEEV | 250 | 283 | 60-183 |
| EEEV | antis_3p_EEEV_V2 | 251 | 475 | 60-375 |
| EEEV | sense_5p_EEEV | 252 | 283 | 100-223 |
| EEEV | sense_5p_EEEV_V2 | 253 | 475 | 100-415 |
| EEEV | sense_3p_EEEV | 254 | 381 | 100-321 |
| VEEV | antis_5p_VEEV | 255 | 135 | 60-35 |
| VEEV | antis_3p_VEEV | 256 | 373 | 600-273 |
| VEEV | antis_3p_VEEV_V2 | 257 | 477 | 60-377 |
| VEEV | sense_5p_VEEV | 258 | 373 | 100-323 |
| VEEV | sense_5p_VEEV_V2 | 259 | 477 | 100-427 |
| VEEV | sense_3p_VEEV | 260 | 144 | 60-84 |
| WEEV | antis_5p_WEEV | 261 | 315 | 60-215 |
| WEEV | antis_3p_WEEV | 262 | 266 | 60-166 |
| WEEV | antis_3p_WEEV_V2 | 263 | 479 | 60-379 |
| WEEV | sense_5p_WEEV | 264 | 266 | 100-216 |
| WEEV | sense_5p_WEEV_V2 | 265 | 479 | 100-429 |
| WEEV | sense_3p_WEEV | 266 | 322 | 60-262 |

As will be evident to one of ordinary skill in the art, the nucleotide positions indicated as allowing for variation correspond to the nucleotide position in the reference sequence provided for the row. For example, for the first row, any of the nucleotides of positions 14-37 of antis 5p IAV (SEQ ID NO: 1) may be varied. In some embodiments, the encrypted RNA comprises one or more variations at any of the positions indicated in Table 6. Further exemplary variations are provided in Table 17.

### Proteins of Interest

The terms “polypeptide”, “peptide”, “amino acid sequence” and “protein” are used interchangeably herein to refer to polymers of amino acids of any length. The polymer may be linear or branched.

In some embodiments, a therapeutic polypeptide of interest comprises a “protein that causes cell death”. A protein that causes cell death, when produced at a sufficient concentration within a cell, increases the death rate of the cell and therefore reduces the expected lifetime of the cell. Proteins that cause cell death include, but are not limited to: granzymes, including Granzyme A and Granzyme K; perforins; pro-apoptotic members of the BCL-2 family such as BCL-2 homology domain 3-only proteins, the B-cell lymphoma-2 (Bcl-2) family proteins BIM, p53 upregulated modulator of apoptosis (PUMA), Bid, Bcl2 modifying factor (BMF), Noxa, Bcl-2-interacting killer (BIK), BCL2 associated X, apoptosis regulator (BAX), Bcl-2 agonist of cell death (BAD); herpesvirus thymidine kinase; Vaccinia virus E3L; Receptor-interacting protein kinase 3 (RIPK3)/mixed lineage kinase domain-like protein (MLKL); caspases, including caspase-3, caspase-6, and caspase-7; gasdermin D.

In some embodiments, a protein that causes cell death can further increase the death rate of a cell when the surrounding milieu contains a sufficient concentration of a molecule that is contacted by the protein that causes cell death to produce a new cytotoxic molecule. Such a potentiating protein could include the use of herpesvirus thymidine kinase in conjunction with ganciclovir.

As used herein, an “immune response” may be a specific reaction of the adaptive immune system to a particular antigen (i.e., a specific or adaptive immune response), a nonspecific reaction of the innate immune system (i.e., a nonspecific or innate immune response), or a combination thereof.

As used herein, “immunogenicity” refers to the capacity of a polynucleotide of the present disclosure to induce an immune response. Some embodiments described herein involve using RNA constructs that have altered nucleotides to reduce the immunogenicity of the polynucleotide in the absence of activation by a translation activator. An aspect of the present disclosure is the discovery that polynucleotides having L and R regions containing modified nucleotides can still be recognized and efficiently replicated by a viral polymerase.

This discovery is surprising, in part because nucleotide modifications can alter the secondary or tertiary structures of a polynucleotide, and these secondary and tertiary structures are important for the polynucleotide to react with and initiate polymerase activity. In addition, there are prior art reports that a viral RdRp, specifically an alphavirus RdRp, cannot replicate templates without unmodified uridine nucleotides for efficient protein translation (see, e.g., Beissert T et al., A Trans-amplifying RNA Vaccine Strategy for Induction of Potent Protective Immunity. Mol Ther. (2020) 28 (1): 119-128).

We note that an attempt to use a nucleoside-modified alphavirus replicon (“a self-amplifying RNA”) encoding a SARS-COV-2 vaccine antigen resulted in loss of antigen (protein) production in vivo (Voigt, E. A., et al. A self-amplifying RNA vaccine against COVID-19 with long-term room-temperature stability. Npj Vaccines (2022). DOI: 10.1038/s41541-022-00549-y). In contrast, in some embodiments, the encrypted RNAs described herein can be developed with 100% of uridine nucleotides modified and can retain at least equal or higher protein production in the presence of a viral polymerase—in addition to substantially reduced immunogenicity in the absence of the viral polymerase. It is recognized that some viral RNAs are modified by cellular enzymes in select positions during their natural lifecycle. As an example, adenosines of Hepatitis C Virus, Zika virus, and Feline leukemia virus are post-transcriptionally modified to N6-methyladenosine by cellular methyltransferases (Gokhale N. & Horner S; PLOS Pathog. 2017 March; 13 (3): e1006188). Modification is thought to be rare, for example the 5 kb RNA genome of Rous sarcoma virus is modified at just 15 positions (~1% of the positions in the transcript). As used herein, an “immunomodulatory polypeptide” refers to a polypeptide which is able to alter an immune response, including by: inducing or suppressing maturation of immune cells, inducing or suppressing cytokine biosynthesis, or altering humoral immunity by stimulating antibody production by B cells. Immunomodulatory polypeptides may have antiviral and antitumor activity, and may also down-regulate other aspects of the immune response, for example shifting the immune response away from a TH2 immune response, which is useful for treating a wide range of TH2-mediated diseases.

In some embodiments, the polypeptide of interest is an immunomodulatory polypeptide. In some further embodiments, the polypeptide of interest is an immunomodulatory polypeptide that is immunogenic in a subject, i.e., acts as an antigen in the subject to yield an immune response. As used herein, the term “antigen” means an immunogenic compound that elicits an adaptive immune response in a subject being treated with the antigen. In particular, an “antigen” relates to any substance that induces in the subject being treated an antigen-specific antibody or T-lymphocyte (T-cell) response. The term “antigen” comprises any molecule which comprises at least one epitope. In one aspect, an antigen is a molecule which, optionally after processing, induces an immune reaction, which is specific for the antigen in the subject being treated. Antigens may include polypeptides derived from allergens, viruses, bacteria, fungi, parasites and other infectious agents and pathogens or from cancers, including tumor antigens. In one aspect, an antigen corresponds to a naturally occurring product, for example, a polypeptide naturally displayed on the surface of a cell, a pathogen, a bacterium, a virus, a fungus, a parasite, an allergen, or a tumor. The antigen may elicit an immune response against a cell, a pathogen, a bacterium, a virus, a fungus, a parasite, an allergen, or a tumor.

The term “pathogen” refers to pathogenic biological material capable of causing disease in an organism. Pathogens include microorganisms such as bacteria, unicellular eukaryotic organisms (protozoa), fungi, as well as viruses.

In some embodiments, the polypeptide of interest comprises an antigen suitable for vaccination of a target organism. In some embodiments, an antigen is selected from the group comprising a self-antigen and non-self-antigen. A non-self-antigen may be a viral antigen, a bacterial antigen, a fungal antigen, an allergen or a parasite antigen.

In some embodiments, the antigen is a self-antigen, particularly a tumor antigen. The term “tumor antigen” or “tumor-associated antigen” refers to proteins that, under normal conditions, are specifically expressed in a limited number of tissues or organs or in specific developmental stages, for example, the tumor antigen may be under normal conditions specifically expressed in stomach tissue, for example in the gastric mucosa, in reproductive organs, e.g., in testis, in trophoblastic tissue, e.g., in placenta, or in germ line cells, and are expressed or aberrantly expressed in one or more tumor or cancer tissues. In this context, “a limited number” can be not more than 3, or not more than 2. Tumor antigens in the context of the present disclosure can include, for example, differentiation antigens, for example cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. In some embodiments, the tumor antigen is associated with the cell surface of a cancer cell and is not or only rarely expressed in normal tissues. In some embodiments, the tumor antigen or the aberrant expression of the tumor antigen identifies cancer cells. In some embodiments, the tumor antigen that is expressed by a cancer cell in a subject, e.g., a patient suffering from a cancer disease, is a self-protein in said subject. In some embodiments, the tumor antigen in the context of the present disclosure is expressed under normal conditions specifically in a tissue or organ that is non-essential, i.e., tissues or organs which when damaged by the immune system do not lead to death of the subject, or in organs or structures of the body which are not or only hardly accessible by the immune system.

As used herein, “background translation” of a polypeptide of interest means the translation of the polypeptide of interest in the absence of a translation activator.

In some embodiments, background translation of the polypeptide of interest is not substantially immunogenic.

In some embodiments, background translation of the polypeptide of interest is not substantially immunogenic; however, translation of the polypeptide of interest in the presence of a translation activator (e.g., in a viral infection or expression of a polynucleotide encoding a translation activator) is substantially immunogenic.

Aspects of the present disclosure relate, at least in part, to an encrypted RNA comprising a coding sequence that encodes a therapeutic polypeptide. In some embodiments, the therapeutic polypeptide is an immunotherapeutic polypeptide. In some embodiments, the immunotherapeutic polypeptide of interest is a chemokine. In some embodiments, the immunotherapeutic polypeptide of interest is a cytokine. In some embodiments, the immunotherapeutic polypeptide of interest is a toll-like receptor (TLR) agonist. In some embodiments, the therapeutic polypeptide of interest is a structural protein. In some embodiments, the therapeutic polypeptide of interest is a blood protein. In some embodiments, the immunotherapeutic polypeptide of interest is a programmed cell death polypeptide. In some embodiments, the immunotherapeutic polypeptide of interest is an antigen. In some embodiments, the immunotherapeutic polypeptide of interest is an antibody. In some embodiments, the therapeutic polypeptide of interest is an endocrine polypeptide. In some embodiments, the therapeutic polypeptide of interest is a human peptide. In some embodiments, the therapeutic polypeptide of interest is a receptor. In some embodiments, the therapeutic polypeptide of interest is a binding protein. In some embodiments, the therapeutic polypeptide of interest is a transcription factor. In some embodiments, the therapeutic polypeptide of interest is a tumor suppressor protein. In some embodiments, the therapeutic polypeptide of interest is a T cell receptor protein. In some embodiments, the therapeutic polypeptide of interest is a homing receptor. In some embodiments the therapeutic polypeptide of interest is a translation factor. In some embodiments, the therapeutic polypeptide of interest is a membrane transporter. In some embodiments, the therapeutic peptide of interest is not a viral peptide or of viral origin.

In some embodiments, the immunotherapeutic polypeptide is a chemokine. The term “chemokine,” as used herein, refers to a peptide that acts as a chemoattractant to guide the migration of cells. In some embodiments, the chemokine is chemokine ligand 1 (CCL1), CCL2, CCL3, CCL4, CCL5, CCL7, CCL8, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL16, XCL1, XCL2, CX3CL1, IL-8, MCP-1, MIP-1alpha.

In some embodiments, the immunotherapeutic polypeptide of interest is a cytokine. The term “cytokine,” as used herein, refers to a peptide that is a signaling protein that helps control immune system responses. In some embodiments, the cytokine is Human tumor necrosis factor precursor (Homo sapiens), TNFα, TNFβ, Prostaglandin, RANKL, VEGF, selectin, addressin. In some embodiments, the cytokine is an interleukin. The term “interleukin,” as used herein, refers to a group of cytokines that are expressed and secreted by body cells. In some embodiments, the interleukin is interleukin-1 (IL-1), IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, Human interleukin-1 receptor antagonist precursor (Homo sapiens), Human interleukin-12A and interleukin-12B precursors (bi-cistronic via P2A site) (Homo sapiens), Human interleukin-2 precursor (Homo sapiens), Mouse interleukin-12A and interleukin-12B precursors (bicistronic via P2A sequence) (Mus musculus), Mouse interleukin-2 precursor (Mus musculus). In some embodiments, the cytokine is an interferon. The term “interferon,” as used herein, refers to a group of cytokines that are expressed and secreted by body cells in response to infection. In some embodiments, the interleukin is Human interferon-beta precursor (Homo sapiens), Human interferon-lambda 3 precursor (Homo sapiens), Human interferon-lambda 3 precursor (Homo sapiens), Human interferon-lambda precursor (Homo sapiens), Mouse interferon-kappa precursor (Mus musculus), Mouse interferon-lambda 2 precursor (Mus musculus), Mouse interferon-lambda 3 precursor (Mus musculus), Syrian hamster interferon-beta 1 precursor (Mesocricetus auratus), Syrian hamster interferon-lambda 3 precursor (Mesocricetus auratus), Cotton rat interferon-alpha precursor (Sigmodon hispidus), Cotton rat interferon-beta 1 precursor (Sigmodon hispidus), Cotton rat interferon-lambda 2 precursor (Sigmodon hispidus), Domestic ferret interferon beta 1 precursor (Mustela putorius furo), IFNα1, IFNα2, IFNα4, IFNα5, IFNα6, IFNα7, IFNα8, IFNα10, IFNα13, IFNα14, IFNα16, IFNα17, IFNα21, IFNβ1, IFN-ω, IFN-γ, IFN-κ, IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-β1, IFN-ε, IFN-κ, IFN-ω1, IFN-γ, IFN-λ1 (IL28A), IFN-λ2 (IL28B), IFN-λ3 (IL29), or IFN-λ4. In some embodiments, the cytokine is a colony stimulating factor (CSF). The term “colony stimulating factor,” as used herein, refers to a secreted glycoproteins that bind to receptor proteins on the surfaces of hemopoietic stem cells. In some embodiments, the colony stimulating factor is CSF-1, CSF-2, CSF-3, CSF-4, CSF-5, CSF-6, G-CSF, GM-CSF, or M-CSF. In some embodiments, the immunotherapeutic polypeptide of interest is a toll-like receptor (TLR) agonist. In some embodiments, the TLR agonist is of bacterial origin. In some embodiments, the TLR agonist that is of bacterial origin is BCSP31, FHA, MOMP, FomA, MymA (Rv3083), ESAT6, PorB, PVL, Porin, OmpA, PepO, OmpU, or flagellins. In some embodiments, the TLR agonist is of viral origin. In some embodiments, the TLR agonist that is of viral origin is glycoprotein F, envelope glycoprotein, glycoprotein GP, NS3, hemagglutinin H, gB, gH, gL, gp120, gp41, p24, or p17. In some embodiments, the immunotherapeutic polypeptide of interest is an interferon stimulated gene. In some embodiments, the interferon stimulated gene is BST2 (tetherin), C6orf150 (MB21D1), DDX58, EIFAK2, HPSE, IFIH1 (MDA5), IFIT1, IFIT2, IFIT3, IFIT5, IFITM1, IFITM2, IFITM3, IRF1, IRF7, ISG15, ISGS20, MX1, MX2, NAMPT (PBEF1), OAS1, OAS2, OAS3, RSAD2 (viperin), or ZC3HAV1 (ZAP).

In some embodiments, the therapeutic polypeptide of interest is a structural protein. In some embodiments, the structural protein is collagen, fibrin, fibrinogen, elastin, tubulin, actin, or myosin.

In some embodiments, the therapeutic polypeptide of interest is a blood protein. The term “blood protein,” as used herein, refers to a protein present in blood plasma. In some embodiments, the blood protein is thrombin, serum albumin, Factor VII, Factor VIII, insulin, Factor IX, Factor X, tissue plasminogen activator, protein C, von Willebrand factor, antithrombin III, glucocerebrosidase, erythropoietin granulocyte colony stimulating factor (GCSF) or modified Factor VIII, or anticoagulants.

In some embodiments, the immunotherapeutic polypeptide of interest is a programmed cell death polypeptide. The term “programmed cell death polypeptide,” as used herein, refers to a polypeptide that initiates a series of molecular steps in a cell that lead to its death. In some embodiments, the programmed cell death polypeptide is a granzyme. In some embodiments the granzyme is Granzyme A. In some embodiments the granzyme is Granzyme K. In some embodiments, the programmed cell death polypeptide is a perform. In some embodiments, the programmed cell death polypeptide is an apoptotic protein. The term “apoptotic protein,” as used herein, refers to a protein is involved in cell death. In some embodiments, the apoptotic protein is a BCL-2 homology domain 3-only protein, BIM, PUMA, BID, BMF, NOXA, BIK, BAD, herpes thymidine kinase, Vaccinia virus E3L, RIPK3/MLKL, caspases, including caspase-3, caspase-6, and caspase-7, or gasdermin D.

In some embodiments, the immunotherapeutic polypeptide of interest is an antigen. The term “antigen,” as used herein, refers to a molecule or moiety or matter that can bind to a specific antibody or T-cell receptor to elicit an immune response. In some embodiments, the antigen is a tumor antigen. In some embodiments the tumor antigen is a alphafetoprotein (AFP), carcinoembryonic antigen (CEA), MAGE, BAGE, GAGE, NY-ESO-1, HER2, HPV16 E7, WT1, MART-1, gp100, tyrosinase, URLC10, VEGFR1, VEGFR2, MUC1, MUC2, surviving, TRP1/gp75, TRP2, gangliosides, PSMA, or EphA3.

In some embodiments, the immunotherapeutic polypeptide of interest is an antibody. The term “antibody,” as used herein, refers to a peptide that is used by the immune system to identify and counteract foreign objects. In some embodiments, the antibody is a therapy for cancer. In some embodiments the cancer antibody is trastuzumab, pembrolizumab, bevacizumab, cetuximab, ibritumomab, ofatumumab, or obinutuzumab. In some embodiments, the antibody is a therapy for viral infections. In some embodiments the viral antibody is ansuvimab, atoltivimab, maftivimab, odesivimab, ibalizumab, obiltoxaximab, raxibacumab, sotrovimab, tixagevimab, cilgavimab, palivizumab, or immunoglobulins. In some embodiments the antibody is a therapy for autoimmune disorders. In some embodiments the autoimmune antibody is clazakizumab, clenoliximab, fezakinumab, fletikumab, gimsilumab, guselkumab, rituximab, or denosumab.

In some embodiments, the therapeutic polypeptide of interest is an endocrine polypeptide. In some embodiments, the endocrine polypeptide is a hormone. In some embodiments, the hormone is insulin, erythropoietin, thyroid hormone, catecholamines, gonadotropins, trophic hormones, human grown hormone, prolactin, oxytocin, dopamine, bovine somatotropin, leptins, adrenocorticotropic hormone (ACTH), adropin, amylin, angiotensin, atrial natriuretic peptide (ANP), calcitonin, cholecystokinin (CCK), gastrin, ghrelin, glucagon, follicle-stimulating hormone (FSH), luteinizing hormone (LH), melanocyte-stimulating hormone (MSH), parathyroid hormone (PTH), Renin, somatostatin, thyrotropin-releasing hormone (TRH), or vasopressin. In some embodiments, the therapeutic polypeptide of interest is an endocrine polypeptide. In some embodiments, the endocrine polypeptide is a growth factor. In some embodiments, the growth factor is epidermal growth factor (EGF), nerve growth factor (NGF), insulin-like growth factor, fibroblast growth factor (FGF), or platelet-derived growth factor (PDGF). In some embodiments, the therapeutic polypeptide of interest is an endocrine polypeptide. In some embodiments, the endocrine polypeptide is a growth factor receptor. In some embodiments, the growth factor receptor is WNT receptor, tie, neurotrophin receptor, ephrin receptor, insulin-like growth factor receptor (IGF receptor), epidermal growth factor receptor (EGF receptor), fibroblast growth factor receptor (FGF receptor), platelet-derived growth factor receptor (PDGF receptor) or vascular endothelial growth factor receptor (VEGF receptor).

In some embodiments, the endocrine polypeptide is an enzyme. In some embodiments, the enzyme is tissue plasminogen activator, streptokinase, cholesterol biosynthetic or degradative, steroidogenic enzymes, kinases, phosphodiesterases, methylases, de-methylases, dehydrogenases, cellulases, proteases, lipases, phospholipases, aromatases, cytochromes, adenylate or guanylate cyclases, or neuraminidases.

In some embodiments, the therapeutic polypeptide of interest is a human peptide. In some embodiments, the human peptide is for gene therapy. In some embodiments the human peptide for gene therapy is PCCA, PCCB, MMUT, MMAA, MMAB, MMADHC, MCEE, IVD, MCCC1, MCCC2, HMGCL, holocarboxylase synthetase, ACAT1, glutaryl-CoA dehydrogenase, OCTN2, SLC22A5, MCAD, VLCAD, LCHAD, HADHA, HADHB, argininosuccinate lyase, ASS1, SLC25A13, BCKDHA, BCKDHB, DBT, CBS, MTHFR, MTR, MTRR, MMADHC, phenylalanine hydroxylase, FAH, TAT, HPD, PAX8, TSHR, DUOX2, SLC5A5, TG, TPO, TSHB, HBB, TRDN.

In some embodiments, the therapeutic polypeptide of interest is a receptor. In some embodiments, the receptor is steroid hormone receptors, peptide receptors, or integrins.

In some embodiments, the therapeutic polypeptide of interest is a binding protein. In some embodiments, the binding protein is growth hormone or growth factor binding protein, single stranded binding protein, calmodulin, gelsolin, polypyrimidine tract-binding protein, maltose binding protein, metallothionein, FABP6, syntaxin binding protein 2, syntaxin binding protein 3, androgen binding protein, TATA-binding protein, LTBP2, E3 binding protein, CREB, retinol binding protein 2, retinol binding protein 4, RNA binding protein FUS, or tropomodulin.

In some embodiments, the therapeutic polypeptide of interest is a transcription factor. In some embodiments, the transcription factor is OCT4, SOX2, KLF4, MYC (OSKM), TFIIA, TFIIB, TFIID, TFIIE, TFIIF, or TFIIH.

In some embodiments, the therapeutic polypeptide of interest is a tumor suppressor protein. The term “tumor suppressor protein,” as used herein, refers to a protein that regulates cell during division and replication. In some embodiments, the tumor suppressor protein is Angiopoietin-2 (Ang2), APC, MADR2, p53, TGF-β, BRCA1, p16, p14, CADM1, or FAS.

In some embodiments, the therapeutic polypeptide of interest is a T cell receptor protein. In some embodiments, the T cell receptor protein is TCR-α, TCR-β, CD2, CD3, CD4, CD5, CD7, CD8, ζ-chain (CD27), or CD28.

In some embodiments, the therapeutic polypeptide of interest is a homing receptor. In some embodiments, the hoking receptor is integrin α4β1, vascular adhesion molecule-1 (VCAM-1), BCR, CD34, or GLYCAM-1.

In some embodiments the therapeutic polypeptide of interest is a translation factor. In some embodiments the translation factor is eIF1A, eIF5B, eIF1, eIF5A, eIF2, or eIF6.

In some embodiments, the therapeutic polypeptide of interest is a membrane transporter. The term “membrane transporter,” as used herein, refers to a membrane protein involved in the movement of ions, small molecules, and macromolecules, such as another protein, across a biological membrane. In some embodiments, the membrane transporter is a sugar transporter. In some embodiments, the sugar transporter is GLUT-1, GLUT-2, GLUT-3, GLUT-4, GLUT-5, GLUT-7, GLUT-9, GLUT-10, GLUT-11, GLUT-12, GLUT-14, SGLT-1, SGLT-2, SGLT-3, SGLT-5, SGLT-6, or HMIT. In some embodiments, the membrane transporter is an amino acid transporter. In some embodiments, the amino acid transporter is CAT-1, CAT-2, CAT-3, SNAT-1, SNAT-2, SNAT-3, SNAT-4, SNAT-5, LAT-2, LAT-4f2 hc, EAAT-1, EAAT-2, EAAT-3, EAAT-4, EAAT-XCT, EAAT-4f2 hc, or TATA-1. In some embodiments, the membrane transporter is a lipid transporter. In some embodiments, the lipid transporter is FABPpm, FATP-1, FATP-2, FATP-3, FATP-4, FATP-5, FATP-6, ABC, or NPC1L1. In some embodiments, the membrane transporter is a nucleoside transporter. In some embodiments, the nucleoside transporter is CNT1, CNT2, ENT-1, ENT-2, ENT-3, or ENT-14.

In some embodiments, the therapeutic polypeptide is a peptide that inhibits viral replication. In some embodiments, the peptide that inhibits viral replication is APOBEC3G, ISG15, OASL. OAS1, OAS2, OAS3, PML (TRIM19), SP100, Tetherin (BST2), Viperin (RSAD2), IFITM1, IFITM2, or IFITM3.

In some embodiments, the coding sequence further comprises a signal peptide. The term “signal peptide,” as used herein, refers to a short peptide bound to another peptide (e.g., a therapeutic peptide or industrial applicable peptide) that translocates another peptide. The terms “signal sequence,” “targeting signal,” “localization signal,” “localization sequence,” “transit peptide,” “leader sequence,” and “leader peptide” are used herein interchangeably. In some embodiments, the signal peptide is Sec/SPI, Sec/SPII, Sec/SPIII, Tat/SPI, or Tat/SPII.

In some embodiments, the nucleotide sequences encoding proteins of interest are listed in Table 3. In some embodiments, the amino acid sequences of proteins of interest are listed in Table 5.

In some embodiments, the therapeutic polypeptide of interest is comprised of a cytokine which is involved in regulating lymphoid homeostasis, such as a cytokine which is involved in and induces or enhances development, priming, expansion, differentiation or survival of T cells. In some embodiments, the cytokine is an interleukin, such as IL-1, IL-2, IL-6, IL-6RA, IL-7, IL-12, IL-15, IL-21, or IL-23. In some embodiments, the interleukin is an anti-inflammatory cytokine, such as IL-1 receptor antagonist (IL-1RN or IL-1RA), IL-23RA, IL-36RA, or IL-37.

In some embodiments, the therapeutic polypeptide of interest is comprised of an “antineoplastic protein”. An antineoplastic protein is a polypeptide effective in the treatment of cancer. Particular classes of antineoplastic proteins include, but are not limited to: monoclonal antibodies, nanobodies, hormones, proteins that cause cell death, immune checkpoint inhibitors, interleukins, and immunogens.

In some aspects, the polypeptide of interest is an antagonist of Programed Cell Death Ligand 1 (PD-L1) or Programmed Cell Death 1 (PD-1).

A PD-1 antagonist, as used herein, is an agent that inhibits or prevents PD-1 activity, e.g., by binding to PD-1.

PD-1 activity may be interfered with by antibodies that bind selectively to and block the activity of PD-1. The activity of PD-1 can also be inhibited or blocked by molecules other than antibodies that bind PD-1. Such molecules include proteins (such as fusion proteins) and peptides, e.g., peptide mimetics of PD-L1 and PD-L2 that bind PD-1 but do not activate PD-1.

Exemplary PD-1 antagonists include those described in U.S. Publications 20130280265, 20130237580, 20130230514, 20130109843, 20130108651, 20130017199, 20120251537, and 20110271358, and in European Patent EP2170959B1, the entire disclosures of which are incorporated herein by reference. Other exemplary PD-1 antagonists are described in Curran et al., PNAS, 107, 4275 (2010); Topalian et al., New Engl. J. Med. 366, 2443 (2012); Brahmer et al., New Engl. J. Med. 366, 2455 (2012); Dolan et al., Cancer Control 21, 3 (2014); and Sunshine et al., Curr. Opin. in Pharmacol. 23 (2015).

Exemplary PD-1 antagonists include: nivolumab (e.g., OPDIVO® from Bristol-Myers Squibb), a fully human IgG4 monoclonal antibody that binds PD-1; pidilizumab (e.g., CT-011 from CureTech), a humanized IgG1 monoclonal antibody that binds PD-1; pembrolizumab (e.g., KEYTRUDA® from Merck), a humanized IgG4-kappa monoclonal antibody that binds PD-1; MEDI-0680 (AstraZeneca/MedImmune) a monoclonal antibody that binds PD-1; and REGN2810 (Regeneron/Sanofi) a monoclonal antibody that binds PD-1. Another exemplary PD-1 antagonist is AMP-224 (Glaxo Smith Kline and Amplimmune), a recombinant fusion protein composed of the extracellular domain of the Programmed Cell Death Ligand 2 (PD-L2) and the Fc region of human IgG1, that binds to PD-1.

A PD-L1 antagonist, as used herein, is an agent that inhibits or prevents PD-L1 activity, e.g., by binding to PD-L1.

PD-L1 activity may be blocked by molecules that selectively bind to and block the activity of PD-L1, e.g. by blocking the interaction with and activation of PD-1 and/or B7-1. The activity of PD-L1 can also be inhibited or blocked by molecules other than antibodies that bind PD-L1. Such molecules include proteins (such as fusion proteins and peptides.

Exemplary PD-L1 antagonists include those described in U.S. Publications 20090055944, 20100203056, 20120039906, 20130045202, 20130309250, and 20160108123, the entire disclosures of which are incorporated herein by reference. Other exemplary PD-L1 antagonists are described in Sunshine et al., Curr. Opin. in Pharmacol. 23 (2015).

PD-L1 antagonists include, for example: atezolizumab (also called MPDL3280A or TECENTRIQ™, Genentech/Roche), an human monoclonal antibody that binds to PD-L1; durvalumab (also called MEDI4736 or IMFINZI™, AstraZeneca/MedImmune), a human immunoglobulin IgG1 kappa monoclonal antibody that binds to PD-L1; BMS-936559 (Bristol-Meyers Squibb), a fully human IgG4 monoclonal antibody that binds to PD-L1; avelumab (also called MSB 0010718C or BAVENCIO®, Merck KGaA/Pfizer), a fully human IgG1 monoclonal antibody that binds to PD-L1; and CA-170 (Aurigene/Curis) a small molecule antagonist of PD-L1.

### RNA Compositions

In some embodiments, an encrypted RNA or DNA encoding an encrypted RNA is complexed with one or more cationic or polycationic compounds, for example with cationic or polycationic polymers, cationic or polycationic peptides or proteins (e.g., protamine), cationic or polycationic polysaccharides, or cationic or polycationic lipids.

As used herein, “lipid nanoparticles” or “LNPs” are nanoscale structures comprised of one or more lipid-like compounds. LNPs include liposomes, lipoplexes, RNA-carrying lipid nanoparticles, DNA-carrying lipid nanoparticles, solid lipid nanoparticles, lipidoid nanoparticles, or cubosomes (see, e.g., Tenchov et al., ACS Nano (2021) 15 (11): 16982-17015; DOI: 10.1021/acsnano.1c04996).

In some embodiments, an encrypted RNA or a DNA encoding an encrypted RNA can be complexed with lipids to form lipid nanoparticles. Therefore, in some embodiments, the composition comprises lipid nanoparticles comprising one or more encrypted RNAs or one or more DNAs encoding encrypted RNAs.

Lipid-based formulations have been increasingly recognized as promising delivery systems for RNA due, in part, to their biocompatibility and their ease of large-scale production.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843).

Cationic (including ionizable cationic) or neutral lipids have been widely studied as synthetic materials for delivery of RNA. In some LNP embodiments, after mixing together, nucleic acids are condensed by lipids to form lipid/nucleic acid complexes. LNP complexes can protect genetic material from the action of nucleases and deliver genetic material into cells by interacting with the negatively charged cell membrane. Some LNPs can be prepared by directly mixing positively charged lipids at physiological pH with negatively charged nucleic acids.

LNPs are typically comprised of four lipid or lipid-like components: (i) a cholesterol or cholesterol derivative; (ii) a cationic lipid, sometimes called an ionizable lipid; (iii) a structural lipid, sometimes called a phospholipid; and (iv) a PEG lipid, sometimes called a PEGylated lipid, which is a polyethylene glycol (PEG) functionalized lipid used to stabilize the particle and improve product stability and pharmacokinetic properties due to surfactant properties (see, e.g., Hou et al, Nat Rev Mater (2021) 6:1078-1094; DOI: 10.1038/s41578-021-00358-0). Furthermore, an LNP can be used for specific targeting by attaching ligands (e.g., antibodies, peptides, or carbohydrates) to its surface or to the terminal end of the attached PEG chains (Front Pharmacol. 2015 Dec. 1; 5:285).

Lipid nanoparticles comprised of cationic lipids have been commonly used non-viral delivery systems for mRNA sequences, as well as oligonucleotides, including plasmid DNA, antisense oligos, or siRNA/small hairpin RNA-shRNA. Cationic lipids, such as DOTAP (1,2-dioleoyl-3-trimethylammonium-propane); DOTMA (N-[1-(2,3-dioleoyloxy) propyl]-N,N,N-trimethyl-ammonium methyl sulfate); or D-Lin-MC3-DMA ((6Z,9Z,28Z,31Z)-heptatriaconta-6,9,28,31-tetraen-19-yl 4-(dimethylamino)butanoate), can form complexes with negatively charged nucleic acids to form nanoparticles by electrostatic interaction, providing high in vitro transfection efficiency. Furthermore, lipid nanoparticles comprised of neutral lipids for RNA delivery have been developed, such as 1,2-dioleoyl-sn-glycero-3-phosphatidylcholine (DOPC)-based liposomes (Adv Drug Deliv Rev. 2014 February; 55:110-115) and newer lipid nanoparticles that utilize squaramide amino lipids (Cornebise et al., Adv Functional Materials (2022) 32 (8): 2016727, DOI: 10.1002/adfm.202106727). Therefore, in some embodiments, an encrypted RNA is complexed with a cationic lipid (e.g., an ionizable cationic lipid) or a neutral lipid, and is thereby formulated into a lipid nanoparticle.

In some embodiments, a composition of the present disclosure comprises an encrypted RNA or a DNA encoding an encrypted RNA that is formulated together with a cationic or polycationic compound or with a polymeric carrier. Accordingly, in a further embodiment, the RNA as defined herein or any other nucleic acid comprised in the inventive (pharmaceutical) composition is associated with or complexed with a cationic or polycationic compound or a polymeric carrier. Therein, the RNA as defined herein or any other nucleic acid comprised in the (pharmaceutical) composition according to the present disclosure can also be associated with a vehicle, transfection, or complexation agent for increasing the transfection efficiency or the expression of the RNA according to the present disclosure or of optionally comprised further included nucleic acids.

As defined above, a polymeric carrier, which may be used to complex any of the RNA described herein or any further nucleic acid comprised in the (pharmaceutical) composition according to the present disclosure may be formed by disulfide-crosslinked cationic (or polycationic) components.

In some embodiments, the composition comprises at least one RNA as defined herein, which is complexed with one or more polycations, and at least one free RNA, wherein the at least one complexed RNA is identical to the at least one free RNA. In this context, the composition can comprise any of the RNA described herein complexed at least partially with a cationic or polycationic compound or a polymeric carrier, e.g., cationic lipids or peptides. In this context, the disclosures of PCT Publication No. WO 2010/037539 and WO 2012/113513 are incorporated herewith by reference. Partially means that only a part of the RNA as defined herein is complexed in the composition with a cationic compound and that the rest of the RNA as defined herein is (comprised in the inventive (pharmaceutical) composition) in uncomplexed form (“free”).

In some embodiments, the complexed RNA in the (pharmaceutical) composition as described herein is prepared according to a first step by complexing any of the RNA described herein with a cationic or polycationic compound or with a polymeric carrier, in a specific ratio to form a stable complex. In this context, no free cationic or polycationic compound or polymeric carrier or only a negligibly small amount thereof remains in the component of the complexed RNA after complexing the RNA. Accordingly, the ratio of the RNA and the cationic or polycationic compound or the polymeric carrier in the component of the complexed RNA can be selected in a range so that the RNA is entirely complexed and no free cationic or polycationic compound or polymeric carrier or only a negligibly small amount thereof remains in the composition.

In other embodiments, the composition comprising the RNA as defined herein may be administered naked without being associated with any further vehicle, transfection, or complexation agent.

In embodiments wherein the (pharmaceutical) composition comprises more than one encrypted nucleic acid species, which may be RNA or DNA encoding an encrypted RNA, these encrypted nucleic acid species may be provided as, for example, two, three, four, five, six, or more separate compositions, which may contain at least one encrypted nucleic acid species each, each encoding a polypeptide of interest. Also, the (pharmaceutical) composition may be a combination of at least two distinct compositions, each composition comprising at least one encrypted nucleic acid. In some embodiments, the two distinct compositions comprise a composition carrying the encrypted RNA or DNA encoding the encrypted RNA and a composition carrying a polynucleotide (an RNA or DNA) encoding a translation activator that activates the encrypted nucleic acid. The (pharmaceutical) composition may be combined to provide one single composition prior to its use or it may be used such that more than one administration is required to administer the (therapeutic) encrypted nucleic acid species. If the (pharmaceutical) composition contains at least one (therapeutic) encrypted nucleic acid species, for example at least two encrypted nucleic acid species, encoding a combination of (therapeutic) proteins, it may e.g. be administered by one single administration (combining all encrypted nucleic acid species), or by at least two separate administrations. Accordingly, any combination of (therapeutic) encrypted nucleic acid species encoding the at least one (therapeutic) polypeptide of interest or any combination of (therapeutic) polypeptides of interest, provided as separate entities (each containing one encrypted nucleic acids species) or as a combined entity (containing more than one encrypted nucleic acid species), is understood as a (pharmaceutical) composition according to the present disclosure. In some embodiments of a (pharmaceutical) composition, at least one encrypted nucleic acid encodes at least two (therapeutic) polypeptides of interest.

The (pharmaceutical) composition according to the present disclosure may be provided in liquid or dry (e.g., lyophilized) form.

In some embodiments, the (pharmaceutical) composition comprises a safe and effective amount of an encrypted nucleic acid, encoding a (therapeutic) polypeptide of interest or a combination of (therapeutic) polypeptides of interest. As used herein, “safe and effective amount” means an amount of the encrypted nucleic acid that is sufficient to significantly favorably affect a disease, disorder, or condition (or a symptom of any thereof). At the same time, however, a “safe and effective amount” is small enough to avoid serious side-effects, that is to say to permit a sensible relationship between advantage and risk. The determination of these limits typically lies within the scope of sensible medical judgment. In relation to the (pharmaceutical) composition of the present disclosure, the expression “safe and effective amount” can mean an amount of the encrypted nucleic acid (and thus of the (therapeutic) polypeptide of interest) that is suitable for obtaining appropriate expression levels of the (therapeutic) polypeptide of interest when the translation activator is present or absent. Such a “safe and effective amount” of the encrypted nucleic acid of the (pharmaceutical) composition may furthermore be selected in dependence on the encrypted nucleic acid species (e.g., nucleoside-modified vs. non-nucleoside-modified, or 5′-triphosphorylated vs. 5′-nonphosphorylated) or on the method of encrypted nucleic acid production, purification, or formulation; for example, since some (therapeutic) encrypted nucleic acid species may lead to substantially higher translation of the (therapeutic) polypeptide of interest than would occur from treatment with an equal amount of an alternative (therapeutic) encrypted nucleic acid species encoding the same (therapeutic) polypeptide of interest. A “safe and effective amount” of the encrypted nucleic acid of the (pharmaceutical) composition as defined above will furthermore vary in connection with: the particular condition to be treated, the severity of the condition, the age and physical condition of the patient to be treated, the duration of the treatment, the nature of any accompanying therapy, the particular pharmaceutically acceptable carrier used, or similar factors within the knowledge and experience of the accompanying doctor. The (pharmaceutical) composition can be used according to the disclosure for human or for veterinary medical purposes.

In some embodiments, the encrypted nucleic acid of the (pharmaceutical) composition, including encrypted RNAs and DNA encoding encrypted RNAs or kit of parts according to the disclosure is provided in lyophilized form. The lyophilized RNA can be reconstituted in a suitable buffer advantageously based on an aqueous carrier prior to administration, e.g., Ringer-Lactate solution, Ringer solution, or a phosphate buffered solution. In some embodiments, the (pharmaceutical) composition or the kit of parts according to the disclosure contains at least two, three, four, five, six, or more encrypted nucleic acids species, which are provided separately inc lyophilized form (optionally together with at least one further additive) and which may be reconstituted separately in a suitable buffer (such as Ringer-Lactate solution) prior to their use so as to allow individual administration of each of the encrypted nucleic acids.

A composition according to the disclosure may contain a pharmaceutically acceptable carrier. The expression “pharmaceutically acceptable carrier” as used herein may include the liquid or non-liquid basis of the composition. If the composition is provided in liquid form, the carrier can be water, typically pyrogen-free water; isotonic saline or buffered (aqueous) solutions, e.g., phosphate, citrate etc. buffered solutions. Particularly for injection of the (pharmaceutical) composition, water or a buffer, such as an aqueous buffer, may be used, containing a sodium salt or a calcium salt or a potassium salt. In some embodiments, the sodium, calcium, or potassium salts may occur in the form of their halogenides, e.g., chlorides, iodides, or bromides, or in the form of their hydroxides, carbonates, hydrogen carbonates, or sulfates, etc. Non-limiting examples of sodium salts include NaCl, NaI, NaBr, Na₂CO₂, NaHCO₂, Na₂SO₄; some examples of the optional potassium salts include KCl, KI, KBr, K₂CO₂, KHCO₂, K₂SO₄; and non-limiting examples of calcium salts include CaCl₂), CaI2, CaBr₂, CaCO₂, CaSO₄, Ca(OH)₂. Furthermore, organic anions of the aforementioned cations may be contained in the buffer. In some embodiments, the buffer suitable for injection purposes as defined above, may contain salts selected from sodium chloride (NaCl), calcium chloride (CaCl₂)), and potassium chloride (KCl), wherein further anions may be present additional to the chlorides. CaCl₂) can also be replaced by another salt like KCl. In some injection buffers, salts are present in a concentration of at least 50 mM sodium chloride (NaCl), and at least 3 mM potassium chloride (KCl) and at least 0.01 mM calcium chloride (CaCl₂)) are present. The injection buffer may be hypertonic, isotonic or hypotonic with reference to the specific reference medium, i.e., the buffer may have a higher, identical, or lower salt content with reference to the specific reference medium, and concentrations of the aforementioned salts may be used that do not lead to damage of cells due to osmosis or other concentration effects. Common buffers or liquids are known to a skilled person. Ringer-Lactate solution is one example of a liquid basis.

Also within the scope of the present disclosure are kits, comprising one or more of the encrypted RNAs described herein or DNA encoding one or more of the encrypted RNAs described herein, or one or more composition thereof. In some embodiments, the kit comprises at least two, three, four, five, six, or more encrypted nucleic acids species. In some embodiments, the kit further comprises instructions for administering to a subject or contacting a cell, tissue, or biological sample with encrypted RNAs described herein or DNA encoding one or more of the encrypted RNAs described herein, or one or more composition thereof. In some embodiments, the kit further comprises a suitable buffer for reconstituting nucleic acids in lyophilized form prior to administration, e.g., Ringer-Lactate solution, Ringer solution, or a phosphate buffered solution.

### Administration and Delivery

Suitable routes of administration include, for example, pulmonary including intratracheal or inhaled, intranasal, oral, rectal, vaginal, transmucosal, or intestinal administration; parenteral delivery, including intradermal, transdermal (topical), intramuscular, subcutaneous, intramedullary injections, as well as intrathecal, direct intraventricular, intravenous, or intraperitoneal. Inhaled administration includes delivery via a nebulizer device or a nasal spray. In some embodiments, inhaled delivery is to airway cells, including upper or lower airway cells. In some embodiments, intramuscular administration is to a muscle selected from the group consisting of skeletal muscle, smooth muscle and cardiac muscle. In some embodiments, intravenous administration results in delivery of RNA to liver cells.

Delivery of an encrypted nucleic acid, such as any of the encrypted RNAs described herein or a DNA encoding any of the encrypted RNAs described herein, can be achieved using viral vectors. Viral vectors useful for delivery include: lentiviral vectors, adenovirus vectors, herpes simplex virus vectors, vaccinia virus vectors, adeno-associated virus vectors, or baculovirus vectors. Within the non-viral subclass of delivery methods, techniques utilizing naked DNA injection, electroporation, biolistic systems for DNA delivery (e.g., gene guns), sonoporation, magnetofection, or LNPs have also been developed for gene delivery.

Lentiviral (LV) vectors based on human immunodeficiency virus type I (HIV-1) have been developed to deliver genetic material to a broad range of cell types. Integration-proficient LV (IPLV) vectors are the conventional form of LV technology, in which vector proviruses permanently integrate into the transduced cell genome. But these integration events sometimes occur within genes, which can dysregulate endogenous gene expression. To minimize integration events, integration-deficient LV (IDLV) vectors have also been developed by mutating the HIV-1 integrase component of LV vectors to ensure that the majority of proviral DNA remains as extrachromosomal episomes. However, some chromosomal integration still occurs with IDLV technology, with 0.1%-1% of proviruses integrating into the genome.

HIV-1-based LV vectors offer a potential means to deliver RNA to a wide range of cell types in vivo and in vitro, as they package their genomes in the form of ssRNA. In conventional LV vectors, the ssRNA genome is reverse-transcribed to give a double-stranded DNA (dsDNA) product, which then enters the nucleus.

Adeno-associated virus (AAV) is a small, helper-dependent, single-stranded DNA virus capable of transducing dividing or non-dividing cells by delivering a predominantly episomal transgene product. In comparison to adenoviral vectors, AAV vectors may provide a safer option for transduction given their potentially diminished pathogenicity and immunogenicity in humans. One disadvantage of AAV vectors is their small transgene capacity of approximately 4.8 kb, which can restrict the breadth of therapeutic genes that may be delivered via AAV vector.

Adenoviral vectors are able to transduce replicating or quiescent cell populations, making them a valuable tool in delivering transgenes in vivo and within mature tissues. AdVs are able to deliver larger transgenes than AAVs; as with AAV, AdV-delivered DNA does not generally integrate into the host genome, but rather, resides episomally in the host nucleus. Such episomal transduction minimizes the risks of insertional mutagenesis, by minimizing direct integration into the host genome. Yet, transgene expression is transient, is vulnerable to cell silencing mechanisms, and is destined for dilution among daughter cells should cell division ensue.

The herpes simplex virus (HSV) is a double-stranded DNA virus capable of delivering up to 50 kbp of transgenic DNA when used as a vector. Similar to the adenovirus, pre-existing immunity to HSV infection is prevalent within the general population; however, HSV vectors are largely able to evade inactivation by host immune response. HSV vector genomes also remain episomal like those of AdVs. As a result, they are expectedly burdened by the same limitations of transient expression faced by AdVs.

RNA delivery offers a means to transiently express exogenous genes in a target cell, as the delivered RNA often remains extranuclear. Non-viral vectors have been developed for in vivo RNA delivery, but tissue-specific targeting requires further optimization.

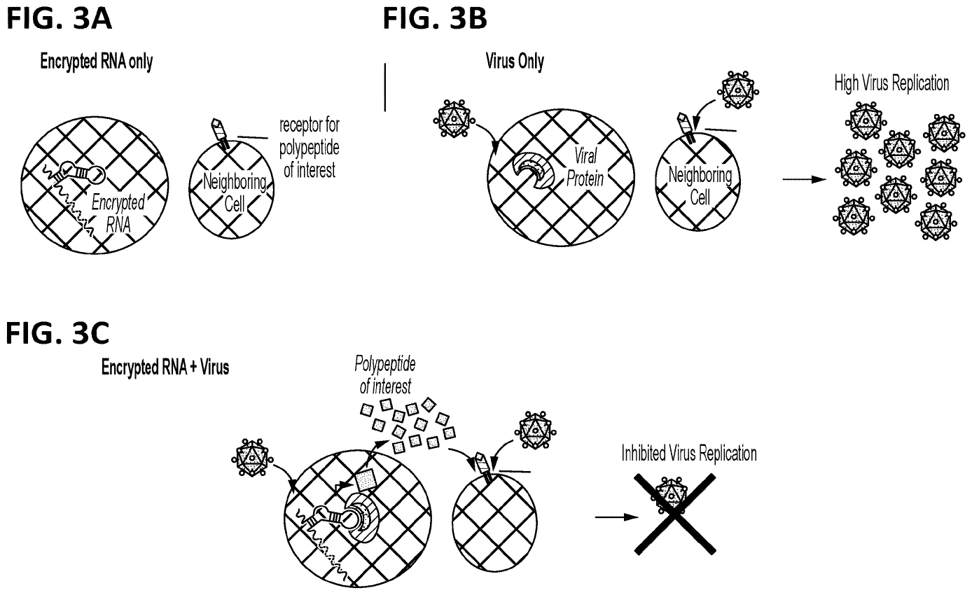

In some embodiments, any of the encrypted RNAs or DNA encoding any of the encrypted RNAs described herein is used to produce a medicament in the context of a viral infection, wherein the medicament is for treatment or prophylaxis of a disease, disorder, or condition caused by the viral infection. As shown in FIG. 3A, in some embodiments, treatment of a cell with a therapeutic encrypted RNA (or a DNA encoding a therapeutic encrypted RNA) in the absence of viral infection (e.g., prophylactic administration) does not result in translation of the therapeutic polypeptide of interest, because no translation activator of the encrypted RNA is present. Conversely, FIG. 3B shows that viral infection of a cell in the absence treatment with a therapeutic encrypted RNA (or a DNA encoding a therapeutic encrypted RNA) can result in high levels of viral replication. FIG. 3C shows that if cells are both treated with a therapeutic encrypted RNA (or a DNA encoding a therapeutic encrypted RNA) and also infected with a virus encoding a translation activator of the therapeutic encrypted RNA, the therapeutic encrypted RNA can be activated by the virus-encoded translation activator, which results in production of a distinct mRNA species and subsequent translation of the therapeutic polypeptide of interest (e.g., an interferon). In some embodiments, upon infection of encrypted RNA treated cells by a virus encoding a translation activator, the therapeutic polypeptide(s) of interest are both translated and secreted to protect neighboring cells from virus infection (including cells that did not initially receive the encrypted nucleic acid treatment), to thereby inhibit virus spread across a subject, e.g., via paracrine signaling.

In some embodiments, any of the encrypted RNAs or DNA encoding any of the encrypted RNAs described herein may be administered to a subject prophylactically, such as in the absence of a viral infection. In some embodiments, the subject is administered a therapeutically effective amount of the encrypted RNAs or DNA encoding any of the encrypted RNAs and administering a second polynucleotide encoding a polymerase capable of interacting with and initiating the transcription or translation of the therapeutic polypeptide or polypeptide. In some embodiments, the method further comprises administering one or more accessory proteins associated with polymerase activity, such as a nucleocapsid protein. In some embodiments, the polymerase and/or accessory proteins are administered in the form of one or more nucleic acid encoding the polymerase and/or accessory proteins.

In some embodiments, the encrypted RNAs or DNAs encoding the encrypted RNAs is administered sequentially or simultaneously as a polynucleotide encoding a polymerase. In some embodiments, the encrypted RNAs or DNAs encoding the encrypted RNAs are present on the same polynucleotide as a polynucleotide encoding a polymerase. In some embodiments, the encrypted RNAs or DNA encoding the encrypted RNAs and a polynucleotide encoding a polymerase are present on separate polynucleotides.

In some embodiments, encrypted nucleic acids or formulations of encrypted nucleic acids (e.g., LNP or viral vector formulations) can be administered systemically or locally. Examples of systemic administration are described above (e.g., intravenous or subcutaneous administration), and can be useful for delivering to regions of the body such as the liver. Alternatively or additionally, the encrypted nucleic acids described herein and compositions thereof may be administered in a local rather than systemic manner, for example, via injection of the pharmaceutical composition directly into a targeted tissue (e.g., in a sustained release formulation). Local delivery can be affected in various ways, depending on the tissue to be targeted. For example, aerosols containing compositions of the present disclosure can be inhaled (for nasal, tracheal, or bronchial delivery); compositions of the present disclosure can be injected into the site of injury, disease manifestation, or pain, for example; compositions can be provided in lozenges for oral, tracheal, or esophageal application; can be supplied in liquid, tablet or capsule form for administration to the stomach or intestines; can be supplied in suppository form for rectal or vaginal application; or can be delivered to the eye by use of creams, drops, or injection. Formulations containing provided compositions complexed with therapeutic molecules or ligands can be surgically administered, for example in association with a polymer or other structure or substance that can allow the compositions to diffuse from the site of implantation to surrounding cells. Alternatively, they can be applied surgically without the use of polymers or supports.

In some embodiments, a subject is treated with a therapeutically effective amount of one or more encrypted RNAs. As described herein, treatment could occur via a variety of means, including by providing therapeutic encrypted RNAs directly in RNA form via formulation into suitable lipid nanoparticles or by providing suitable DNAs encoding for the encrypted RNA as plasmids or viral vectors.

In some embodiments, a subject is treated with a therapeutically effective amount of more than one encrypted RNA, wherein treatment with each encrypted RNA can be accomplished by the same delivery method or by different delivery methods or by combinations thereof. If more than one encrypted RNA is delivered in DNA encoded form, each encrypted RNA could be encoded in a unique DNA molecule, or more than one encrypted RNA could be encoded in a single DNA molecule.

In some embodiments, a subject is treated with more than one therapeutic encrypted RNA, wherein at least two therapeutic encrypted RNAs encode different polypeptides of interest.

In some embodiments, a subject is treated with more than one therapeutic encrypted RNA, wherein at least two therapeutic encrypted RNAs have template regions for binding (interacting with) the same translation activator.

In some embodiments, a subject is treated with more than one therapeutic encrypted RNA, wherein at least two therapeutic encrypted RNAs have template regions for binding (interacting with) different translation activators.

In some embodiments, a cell harboring at least one encrypted RNA is created by delivering one or more encrypted nucleic acids encoding one or more therapeutic polypeptides of interest into a cell. In some further embodiments, at least two of these encrypted RNAs encode different therapeutic polypeptides of interest. In some further embodiments, at least two therapeutic encrypted RNAs have unique template regions for binding (interacting with) the same translation activator. In some further embodiments, at least two therapeutic encrypted RNAs have template regions for binding (interacting with) different translation activators.

In some embodiments, the present disclosure comprises a plant or animal cell comprising an encrypted nucleic acid. For example, the encrypted nucleic acid of the present disclosure may be exogenous to the plant or animal cell, e.g., an encrypted nucleic acid in which the polypeptide of interest is an antiviral protein.

### EXAMPLES

The invention is not limited in its application to the details of construction or to the arrangement of components that are set forth in the foregoing description or illustrated in the drawings. The invention is capable of other embodiments and of being practiced or of being carried out in various ways. It should also be understood that, unless clearly indicated to the contrary, in any methods claimed herein that include more than one step or act, the order of the steps or acts of the method is not necessarily limited to the order in which the steps or acts of the method are recited.

While several inventive embodiments are described and illustrated herein, those of ordinary skill in the art will readily envision a variety of other means or structures for performing the function or obtaining the results or one or more of the advantages described herein, and each of such variations or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, or configurations will depend upon the specific application or applications for which the inventive teachings are used.

Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed.

Inventive embodiments of the present disclosure are directed to each individual feature, system, article, material, kit, or method described herein. In addition, any combination of two or more such features, systems, articles, materials, kits, or methods, if such features, systems, articles, materials, kits, or methods are not mutually inconsistent, is included within the inventive scope of the present disclosure.

Other aspects of the invention will be apparent to those skilled in the art from consideration of the specification and practice of the invention disclosed herein. It is intended that the specification and examples be considered as exemplary only, with a true scope and spirit of the invention being indicated by the following claims. All of the claims in the claim listing are herein incorporated by reference into the specification in their entireties as additional embodiments.

All definitions, as defined and used herein, should be understood to control over dictionary definitions, definitions in documents incorporated by reference, or ordinary meanings of the defined terms. All references, patents, and patent applications disclosed herein are incorporated by reference with respect to the subject matter for which each is cited, which in some cases may encompass the entirety of a cited document.

The present invention is further illustrated by the following examples, which in no way should be construed as further limiting.

### Conventions

Unless otherwise noted, the following buffers have the below essential compositions:
WFI is nuclease-free, endotoxin-free water for injection
PBS is phosphate buffered saline (0.144 g/L KCl; 9.00 g/L NaCl; 0.795 g/L Na₂HPO₄-7H₂O)
DPBS is Dulbecco's Phosphate-Buffered Saline (0.10 g/L anhydrous CaCl₂); 0.20 g/L KCl; 0.20 g/L KH₂PO₄; 0.10 g/L MgCl₂·6H₂O; 8.00 g/L NaCl; anhydrous Na₂HPO₄; 2.1716 g/L Na₂HPO₄-7H₂O; pH 7.4)
DPBS-CMF is calcium and magnesium-free DPBS (0.10 g/L anhydrous CaCl₂); 0.20 g/L KCl; 0.20 g/L KH₂PO₄; 8.00 g/L NaCl; anhydrous Na₂HPO₄; 2.1716 g/L Na₂HPO₄·7H₂O; pH 7.4)
DMEM is Dulbecco's Modified Eagle Medium using the Corning formulation
D02 is Dulbecco's Modified Eagle Medium supplemented with 2% (v/v) Fetal Bovine Serum, 100 U/mL penicillin, and 100 μg/mL of streptomycin (all concentrations final).
D10 is Dulbecco's Modified Eagle Medium supplemented with 10% (v/v) Fetal Bovine Serum, 100 U/mL penicillin, and 100 μg/mL of streptomycin (all concentrations final).
MEM is minimal essential media
Oxoid agar is Oxoid™ Purified Agar (Thermo Fisher Scientific, cat. No. LP0028B)
sterile saline is 0.9 g/L NaCl in WFI
100% ethanol or absolute ethanol is >99.5% ethanol (e.g., MilliporeSigma, cat. No 459836)
70% ethanol is 70% (v/v) ethanol in NFW (see below)
dH₂O is nuclease-free water with resistivity >18.0 megaohm·cm
NFW is dH₂O that is certified nuclease-free (DNAse-free and RNAse-free)
PFA is paraformaldehyde

Unless otherwise noted, water-soluble solutions were usually prepared in dH₂O.

PCR means polymerase chain reaction; RT means reverse transcription; RT-qPCR means reverse-transcription quantitative polymerase chain reaction; Cq is the cycle of quantification, in qPCR or RT-qPCR the cycle at which the reaction signal exceeds a threshold. Unless otherwise indicated, all reactions usually occurred at a pressure of 89-101 kPa.

Room temperature is any temperature in the interval from 19 to 26° C.

### Nanoparticle Formulation

FM00 is DPBS
FM01 is 5% (m/v) sucrose in DPBS-CMF

### Immunostaining

PBSM is DPBS-CMF+5% (m/v) dry nonfat milk
PBSMT is PBSM with 0.05% (v/v) Tween-20
PBST is DPBS-CMF+0.05% (v/v) Tween-20
   Other conventions
Opti-MEM means Opti-MEM I Reduced Serum Media (Thermo Fisher Scientific, cat. No. 31985070)
TPCK-trypsin means modified trypsin (obtained from bovine pancreas) that has been treated with N-tosyl-L-phenylalanine chloromethyl ketone (TPCK) to inactivate extraneous chymotryptic activity (Millipore Sigma, cat. No. T8802-100 MG)
BSA means globulin-free bovine serum albumin, typically sourced as a 35% (m/v) sterile solution from (MP Biomedicals; cat. No. 08810061)

### Additional Definitions

dpi means days post-infection
hpi means hours post-infection
PFU means Plaque Forming Unit
FFU means Focus Forming Unit
PFU and FFU, as measures of the level of infectious virus, are used interchangeably, whether a plaque assay or focus-forming unit assay was performed.
Unless otherwise specified, when n is any non-negative number, n log means 10ⁿ (e.g., 2 log means 100, and 3 log means 1000).
“AU” means arbitrary units. This can correspond to “raw data” as obtained from a raw measurement (e.g. one reported by an instrument) or to consistently transformed “raw data” (e.g., data consistently normalized by subtracting a value or by dividing by a value) to aid comparison within an experiment or between experiments.
“~” means approximately equal to.
Unless otherwise noted, GFP or green fluorescent protein means any protein that exhibits green fluorescence in cells that are exposed to light in the blue to ultraviolet range and can thereby be used to report on the level of protein translation in cells.
Unless otherwise noted, RFP or red fluorescent protein means any protein that exhibits red to orange fluorescence in cells and can thereby be used to report on the level of protein translation in cells.

### Virus Strains

To aid in the teaching of these Examples, example viral strains or example virus isolates are provided that in no way limit the scope of the invention disclosed. Although particular strains or isolates are described here, other strains, species, or genera of virus may be used, as appropriate.

Influenza A virus abbreviations are: A/PR8 means Influenza A/PuertoRico/8/1934 (H1N1); A/SwOH means Influenza A Virus, A/swine/Ohio/09SW79M/2009 (H3N2); A/NewCaledonia means Influenza A Virus, A/New Caledonia/20/1999 (H1N1).

Influenza B virus abbreviations are: B/Texas means Influenza B Virus, B/Texas/06/2011 (Yamagata Lineage); B/Sydney means Influenza B virus B/Sydney/507/2006 (Yamagata Lineage); B/Brisbane means Influenza B Virus, B/Brisbane/60/2008 (Victoria Lineage); B/Ohio means Influenza B virus B/Ohio/01/2005 (Victoria Lineage).

Coronavirus abbreviations are: OC43 or OC43-CoV means Human Coronavirus OC43 (unless otherwise noted, strain ATCC VR-759 is used for the OC43 experiments herein); SARS-COV-2 means Severe acute respiratory syndrome coronavirus 2; Washington or WA-1 means SARS-COV-2 Isolate USA-WA1/2020 (BEI Resources, cat. No. NR-52281); MA30 means the SARS2-N501YMA30 mouse-adapted SARS-COV-2 virus described in (Wong et al. Nature (2022), DOI: 10.1038/s41586-022-04630-3); Omicron BA.1 means the SARS-CoV-2 isolate from Omicron variant lineage B.1.1.529; Alpha means the SARS-CoV-2 isolate from Alpha variant lineage B.1.1.7; Beta means the SARS-COV-2 isolate from Beta variant lineage B.1.351; Gamma means the SARS-COV-2 isolate from Gamma variant lineage P.1; Delta means the SARS-COV-2 isolate from Delta variant lineage B.1.617.2; MERS-COV means Middle East Respiratory Syndrome Coronavirus (MERS) (unless otherwise noted, isolate EMC/2012 is used for the MERS experiments performed or prophesized herein).

EMCV abbreviations are: “EMCV” means Encephalomyocarditis virus, strain MM (BEI Resources; cat. No. NR-19846).

RSV abbreviations are: “RSV A2” or “A2” means Human Respiratory Syncytial Virus, strain A2; and “RSV B1” or “RB1” means Human Respiratory Syncytial Virus, strain B1/18537.

Human parainfluenza virus abbreviations are: “HPIV1” means human parainfluenza virus 1 and “HPIV3” means human parainfluenza virus 3.

Human metapneumovirus abbreviations are: “HMPV” means human metapneumovirus.

Henipavirus abbreviations are: “NiV” means Nipah virus; “NiV-Bangladesh” or “NiVB” means Nipah virus, Bangladesh strain; “NiV-Malaysia” or “NiVm” means Nipah virus, Malaysia stain; and “HeV” means Hendra virus.

Measurement of LNP Size Via Dynamic Light Scattering & Calculation of Polydispersity Index (PDI) from Particle Size Distribution

Particle size and particle size distribution of LNP formulations were quantified using a standard technique: Dynamic Light Scattering (DLS). DLS was performed using a Zetasizer Pro (Malvern Panalytical, Malvern UK).

Each LNP sample was diluted 100-fold with DPBS-CMF before analysis. Peak size was reported as the average diameter in nanometers (d·nm) for each separate peak of the distribution, as calculated by the first cumulant or moment of the distribution, using the method specified in ISO method ISO22412:2017 (ISO standard on Particle size analysis via Dynamic Light Scattering (DLS)).

Homogeneous, monodisperse preparations were expected to have a low polydispersity index (PDI) (typically PDI<0.1 for monodisperse preparations), where PDI was also determined using the Zetasizer Pro. PDI was determined as the square of the standard deviation divided by the square of the mean particle diameter, and PDI width is the square root of the PDI times the z-average.

### Measurement of Zeta Potential of LNP Mixture

The zeta-potential (ζ-potential) of formulated LNPs was also measured using the Zetasizer Pro. The parameters of the Zetasizer Pro were set as follows: temperature at 25° C., viscosity at 0.8872 (cP), a dielectric constant of 78.6, and Henry function of 1.5. Disposable folded capillary cells (DTS1070, Malvern Instruments) were rinsed thoroughly before use with water, followed by ethanol, and finally water again using a minimum of 1 mL each rinse. After the final rinse, capillary cells were air-dried before use. Each LNP sample was prepared at three concentrations in 0.22 μm filtered 10 mM NaCl. Capillary cells were loaded with 1 mL of diluted LNP sample and the averaged phase (over 5 measurements), frequency, and zeta potential distribution were measured.

### Measurement of RNA Encapsulation Efficiency Via Exclusion of Membrane-Impermeable RNA-Specific Dye

The degree of encapsulation of an RNA within an LNP formulation was estimated by determining the exclusion of a membrane impermeable, RNA-specific dye from RNA formulated into the LNPs when the LNPs are intact vs. when they are disrupted. As an example, concentration of LNP-formulated RNA and efficiency of encapsulation was measured using a membrane-impermeable, RNA-specific RiboGreen dye (Thermo Fisher Scientific, cat. No. R11490). Using this reagent, the concentration and encapsulation efficiency of an LNP-formulated RNA with RiboGreen was measured in duplicate following the manufacturer's directions. The RNA encapsulation efficiency was calculated using the below equation:

$${ω}_{RNA} = \frac{( {F}_{total} - {F}_{u n} )}{{F}_{total}}$$

where ω_{RNA} is the RNA encapsulation efficiency, Fₜₒₜₐₗ is the total RNA fluorescence, and Fᵤₙ is the fluorescence component attributable to the RNA outside of the nanoparticles (‘unencapsulated RNA’). LNP formulations suitable for in vitro and in vivo studies typically have ω_{RNA}>0.85.

### Measurement of pKa via TNS

LNP pKₐ was determined using TNS (6-(p-Toluidino)-2-naphthalenesulfonic acid, Sigma (T9892)) and an assay according to (Zhang et al., Langmuir (2011); DOI: 10.1021/10.1021/la104590k). Briefly, LNPs are diluted to 1 and 10 ng/μL (as determined by the concentration of encapsulated mRNA) in a series of buffers with pH ranging between 3 and 12. Buffered solutions are composed of 150 mM NaCl or 100 mM citric acid/citrate, sodium acetate, N-2-hydroxyethylpiperazine-N′-2-ethanesulfonic acid (HEPES), or 3-morpholinopropane-1-sulfonic acid (MOPS) and 150 mM NaCl. A stock solution of TNS is prepared as a 300 μM solution in DMSO and then added to the above buffered solution containing LNPs to a 6 μM final solution of TNS. The fluorescence of the resulting solution was read on a Spectra Max M5 fluorescence plate reader (Molecular Devices) with the excitation wavelength set at 325 nm and the emission wavelength set at 435 nm. The fluorescence of TNS was plotted against pH and fitted using a three parameter sigmoid function. In the presence of LNPs, TNS fluorescence reaches a maximum when 100% of the amino lipids are ionized, when the amino lipids are in the un-ionized state TNS has little fluorescence. The pH values at which half of the maximum fluorescence is reached is reported as the apparent pKₐ values of the LNP.

### Measurement of Protein Translation Via Flow Cytometry

LNPs containing RNA were applied to adherent cells (50-75% confluency) at a typical dosage of 5-50 ng of mRNA per cm² of plate surface area. At 24 h post mRNA application, adherent cell cultures were first washed with DPBS, then detached from their plastic substrate by enzymatic dissociation with TrypLE (Thermo Fisher Scientific, cat. No. 12604013) for 5 min at 37° C. Dissociated cells were subsequently resuspended in a suitable isotonic buffer (often DPBS-CMF supplemented with 5% FBS and 2 mM EDTA) and loaded into a CytoFlex S flow cytometer (Beckman Coulter).

Typically, a total of about 10,000 live, single-cell events were recorded for analysis. Live cells events were gated on forward-scatter and side-scatter, and single live cells were further gated on forward scatter height vs. forward scatter area. Subgates corresponding to live fluorescent cells populations were further established based on the spectral characteristics of the fluorescent protein utilized. Single cells are gated by forward and side scatter and live cells are further gated by fluorescence (e.g., GFP or RFP) intensity. Fluorescent protein gates were set so that no more than 1% of untreated cells fell within an fluorescent-protein positive gate.

Fluorescence values of cell populations under study were reported as: (i) the median fluorescence intensity of all single, live-cell events; (ii) the fraction of fluorescent-protein positive cells ((fluorescing live cells]/[total live cell population]).

When reported as normalized fluorescent intensity (fold-increase), the median intensity from samples incubated with empty LNP (without RNA) was used as a basis for normalization.

Measurement of Secreted Luciferases which Convert Coelenterazine

Method A: Culture media samples or tissue homogenate were analyzed with luminescence assay by adding 50 μL QUANTI-Luc luciferase substrate (InvivoGen, cat. No. rep-qlc2) to 10 μL of the sample in triplicates and measuring luminescence signal immediately on multimode plate reader (PerkinElmer).

Method B: Luciferase-containing samples were serially diluted by at least 10-fold using DPBS-CMF and 10 μL of the diluted sample were transferred to white opaque 96-well microplates. Immediately before the reading, coelenterazine substrate (GoldBio, cat. No. CZ2.5) was diluted in DPBS to a final concentration of 3.5 μM and 90 μL of the 3.5 UM coelenterazine solution were added to the sample shortly before reading (ideally <5 min). Quantification of luciferase activity as measured by detected chemiluminescence activity was using a PerkinElmer VictorX3 2030 Multilabel Reader.

### ELISA (Human, Mouse, Hamster)

For all ELISAs, a standard curve was created using provided standards in the kit and concentrations of analyte of interest were estimated by fitting a linear least squares regression model to the standard curve and interpolating the test sample analyte concentration from either the absorbance or luminescence of the recorded value.

Human interferon beta secreted into cell culture supernatant was detected using a LumiKine Xpress hIFN-β 2.0 ELISA kit (InvivoGen, cat. No. uex-hifnbv2) according to the manufacturer's instructions. A standard curve was created using provided standards in the kit and concentrations of human IFN-β in picomoles/mL (nM) was estimated as described above. Dilutions of the test samples were made at ratios 1:1, 1:25, 1:50, and 1:100 with the manufacturer's supplied buffer.

Human interferon lambda 1-3 secreted into cell culture supernatant were detected using a DIY Human IFN Lambda 1/2/3 (IL-29/28A/28B) ELISA kit (TCM) (PBL Assay Science, cat. No. 61840-1) according to the manufacturer's instructions. A standard curve was created using provided standards in the kit and concentrations of human IFN-β in picomoles/mL (nM) was estimated. Dilutions of the test samples were made at ratios 1:1, 1:25, 1:50, and 1:100 with the manufacturer's supplied buffer.

The human inflammatory cytokine TNF-α was detected from tissue culture supernatants using the Human TNF-alpha DuoSet ELISA kit (R&D Systems, cat. No. DY210) according to the manufacturer's instructions. Concentrations of secreted TNF-α in the test samples were calculated as described above. Dilutions of the test samples were made at ratios 1:1 with the supplied buffer.

Mouse interferon beta secreted into cell culture supernatant was detected using a LumiKine Xpress mIFNb-2.0 ELISA kit (InvivoGen, cat. No. luex-mifnbv2) according to the manufacturer's instructions. A standard curve was created using provided standards in the kit and concentrations of human IFN-β in picomoles/mL (nM) was estimated. Dilutions of the test samples were made at ratios 1:1, 1:25, and 1:50 with the supplied buffer.

Mouse interferon lambda 2 and 3 secreted into cell culture supernatant were detected using a DIY Mouse IFN Lambda 2/3 (IL-28A/B) ELISA kit (TCM) (PBL Assay Science, cat. No. 62830-1) according to the manufacturer's instructions. A standard curve was created using provided standards in the kit and concentrations of human IFN-β in picomoles/mL (nM) was estimated. Dilutions of the samples were made at ratios 1:1, 1:25, 1:50, 1:100 with the supplied buffer.

The murine inflammatory cytokine TNF-α was detected from tissue culture supernatants using the Mouse TNF-alpha DuoSet ELISA kit (R&D Systems, cat no. DY410) according to the manufacturer's instructions. A standard curve was created using provided standards in the kit and concentrations of human TNF-α in picomoles/mL (nM) was estimated.

Isolation of Cell-Free or Viral RNA from Cell-Free Materials

RNA was extracted from infected cell culture supernatants using the Qiagen Viral RNA Mini Kit (Qiagen, cat. No. 52906) according to the manufacturer's protocol with one exception. During RNA extraction, 5 μL of isolated MS2 bacteriophage genomic RNA (typically <100 μg) was spiked into each mL of the AVL buffer used for lysis as a spike-in standard for downstream quantitation.

### Quantification of Cellular/Tissue Gene Expression Changes Via RT-qPCR

RNA was purified from cultured cells using the Monarch Total RNA Miniprep Kit (New England Biolabs, cat. No. T2010S) according to the manufacturer's protocol with one exception. As DNAse treatment was included as a part of subsequent downstream assays, the optional ‘on-column DNAse treatment’ was omitted.

RNA was purified from animal tissue using one of two methods: (i) Monarch Total RNA Miniprep Kit (New England Biolabs, cat. No. T2010S) according to the manufacturer's protocol, but omitting the optional ‘on-column DNAse treatment’ as described above; (ii) TRIzol Reagent (Thermo Fisher Scientific, cat. No. 15596018) according to the manufacturer's protocol.

Purified cellular RNA, whether obtained from in vitro cultured cells or animal tissues, was subsequently treated with DNAse from the TURBO DNAse-Free kit (Thermo Fisher Scientific, cat. No. AM1907) and the DNAse enzyme removed using the supplied inactivation reagent according to the manufacturer's instructions.

Gene expression changes were quantified by performing reverse transcription quantitative real time PCR (RT-qPCR) on purified RNA isolated from cells. RT-qPCR was performed using either dye-based (e.g. SYBR Green I) or hydrolysis-probe based (e.g., TaqMan probes) chemistries.

Complementary DNA (cDNA) was prepared from DNAse-treated RNA in 20 μL reverse transcription reactions using reagents obtained from ProtoScript® II First Strand cDNA Synthesis Kit (New England Biolabs, cat no. E6560L). First, 6 μL of DNA-free RNA was combined with 2 μL Random Primer Mix and the combined volume heated to 65° C. for 5 min before cooling on ice. Random Primer Mix (60 μM) contains 35 μM random hexamers, 25 μM dT23 VN and 1 mM dNTPs in 5 mM Tris-HCl (pH 8.0) and 0.5 mM EDTA.) Next, 10 μL of ProtoScript® II Reaction mix and 2 μL of ProtoScript® II Enzyme mix were added and the reaction gently mixed. The reverse transcription reaction was carried out for ~1 h according to the manufacturer's protocol (25° C. for 5 min; 42° C. for 1 h; 80° C. for 5 min). The resultant cDNA was used for further qPCR analysis. All cDNA templates were diluted (typically within dilution ratios of 1:2 to 1:10 in NFW) before use in qPCR reactions.

Dye-based qPCR was performed with 20 μL reaction volumes assembled by combining the following for each reaction: 10 μL of 2×Luna® Universal qPCR Master Mix (New England Biolabs. Cat no. M3003E), 5 μL of the diluted cDNA, 2.5 μL of 1 μM forward primer in NFW (125 nM final), 2.5 μL of 1 μM reverse primer in NFW (125 nM final).

Final reactions were set up in a MicroAmp Optical 96-well Reaction Plate (Thermo Fisher Scientific, cat. No. 4306737) and analyzed in an ABI 7300 Real Time PCR machine (Applied Biosystems, Inc.). The thermocycling conditions are listed in Table 7 below:

TABLE 7
Standard qPCR thermocycling conditions

| | | | Number of |
|---|---|---|---|
| Step | Temperature | Duration | Cycles |
| Enzyme activation | 95° C. | 60 s | 1 |
| Denaturation | 95° C. | 15 s | 40 |
| Annealing/Extension | 60° C. | 30-60 s | |

Hydrolysis Probe-Based qPCR

For hydrolysis probe-based qPCR, hydrolysis probes were designed and obtained from MilliporeSigma or Integrated DNA Technologies and diluted if necessary to 100 μM concentration with 10 mM Tris·HCl, pH 8.0 or NFW. A 10× Probe/primer mix was created at the following concentrations for probe and primers: hydrolysis probe (1 μM), forward primer (4 μM), and reverse primer (4 μM), all dissolved in NFW. The final reaction master mix was set up using 2× Luna® Universal Probe qPCR Master Mix (New England Biolabs, cat no. M3004E), 1× the Probe/primer mix, and 5 μL of the diluted cDNA in a total reaction volume of 20 μL.

Final reactions were set up in a MicroAmp Optical 96-well Reaction Plate (Thermo Fisher Scientific, cat. No. 4306737) and analyzed in an ABI 7300 Real Time PCR machine (Applied Biosystems, Inc.). The thermocycling conditions are listed in Table 7 above.

### Influenza Infectious Titer by Focus-Forming Unit Assay

Virus supernatants were serially diluted in DMEM, containing 0.035% BSA, 50 mM sodium bicarbonate, and antibiotics (typically penicillin/streptomycin). Twelve-well dishes of MDCK cells were infected with various serial dilutions of the virus in 400 μL of the above media and incubated at 37° C. in 5% CO₂ for 90 min and gently rocked every 15 min. After removing the inoculum, the wells were overlaid with 2 mL of MEM with 0.001% (m/v) Dextran, 0.1% (m/v) sodium bicarbonate, 1 μg/mL of TPCK-trypsin, and 2% (m/v) low melt Oxoid agar. (The purpose of adding TPCK-Trypsin was to allow for influenza virus to spread cell-to-cell by maturation of the viral hemagglutinin protein.) The plates were then incubated for 48 h in a humidified 5% CO₂ incubator at one of two temperatures: 37° C. (for most influenza A strains) or 33° C. (for most influenza B strains).

After the 48 h incubation period, the plaquing monolayers were fixed using 1 mL of 4% PFA per well for a minimum of 4 hours to overnight (16-20 hours). Following this, the agar overlay plugs were removed by gentle tapping on the side of the microplate and rinsing of the wells with dH₂O. For influenza A strains, plates were incubated with 1:4000 dilution of anti-influenza A antibody (Chicken Influenza A, Puerto Rico 8/34 Polyclonal Antibody; MyBioSource Cat. No. MBS623909) in PBSM for 4 h at room temperature on a shaker. The plates were washed 3× with PBS and incubated with 1:4000 dilution of Peroxide-Conjugated Affinipure Donkey anti-Chicken Antibody (Jackson Immunoresearch, cat no. 703-035-155) in PBSM for 1 h at room temperature on a shaker. Following incubation with the secondary antibody, the wells were washed 3× with PBS and the plaques developed using the TMB Solution (Ready-to-Use) for IMMUNOBLOT (Thermo Fisher Scientific, cat no. 002019).

Viral titers were calculated by enumerating a countable number of discrete infection foci (e.g., >50) and multiplying by the nominal dilution factor. Viral titers were reported as plaque-forming units per mL (PFU/mL), regardless of whether the counting of discrete infection foci was enabled by immunostaining (focus-forming unit; FFU) or by clearing of a cell monolayer (plaque forming unit; PFU).

For Influenza B viruses, plates were incubated with 1:4000 dilution of anti-influenza B Rabbit polyclonal antibody HD09DE2801-B against B/Florida HA (Sino Biological, cat no. 11053-T62) in PBSM for 4 h at room temperature. The plates were washed 3× with PBS and incubated with 1:4000 dilution of Peroxide-Conjugated Affinipure Donkey anti-Rabbit Antibody (Jackson Immunoresearch, cat no. 711-035-152) in PBSM for 1 h at room temperature. Following incubation with the secondary antibody, the plaques were developed, and virus quantified as described above.

### SARS-COV-2 & MERS-COV Infectious Titer Via Plaque Assay

Virus was titrated via plaque assay as described in (Zheng et al., Nature (2021); DOI: 10.1038/s41586-020-2943-z). Briefly, virus or tissue homogenate supernatants were serially diluted in DMEM and then used to inoculate 12 well plates of Vero E6 cells. Cells were incubated with inocula for 1 h at 37° C. in 5% CO₂ humidified incubators, with gently rocking every 15 min. After removing the inocula, plates were overlaid with D02 supplemented with 0.6% (m/v) low-melt agarose. After 3 days, overlays were removed, and plaques visualized by staining with 0.1% crystal violet. Viral titers were recorded as PFU/mL for cell culture samples or PFU/mL/mg tissue for tissue homogenate samples.

### Infectious Titration of Generation-Limited SARS-COV-2

A BSL-2 infection model of SARS-COV-2 (“generation-limited SARS-COV-2” or “SARS2-GL”) was used in some in vitro assays. Titration of infectious generation-limited SARS-COV-2 virus was based upon a viral titration protocol outlined by (Mendoza et al., Curr Protoc Microbiol, (2020); DOI: 10.1002/cpmc.105). Supernatant samples were serially diluted in D02, then 6-well plates of Vero-E6-hACE2+ORF3a/E cells were incubated with the diluted inocula for 1 h at 37° C. in 5% CO₂ humidified incubators, with gently rocking by hand applied every 15 min during the incubation.

After removing the inocula, plates were overlaid with 0.8% (m/v) Oxoid agarose in 1×MEM (ThermoFisher Scientific, cat. No. 11935046) containing 3.5% FBS supplemented with 0.2 μg/mL doxycycline, 10 μg/mL puromycin, 1.8 mM L-glutamine (ThermoFisher Scientific, cat. No. 25030149), 0.9× nonessential amino acids (ThermoFisher Scientific, cat. No. 11140050), 0.7× sodium bicarbonate (ThermoFisher Scientific, cat. No. 25080094). After 48 h, plaques were stained by adding an overlay additionally supplemented by 0.01% (m/v) neutral red. Plaques were enumerated at 24 h after adding the second overlay (at approximately 72 hpi).

### SARS-COV-2 Infectious Titer Via RT-qPCR

SARS-COV-2 viral RNA was extracted from infected cell culture supernatants as described above (“Standard Methods”). Further steps of DNAse treatment and RT-qPCR (dye-based or hydrolysis probe based), were carried out as described above (“Standard Methods). Virus genome titers were obtained by normalizing the viral RNA detected in the supernatant to the MS2 bacteriophage RNA spike-in.

TABLE 8
Primers used for SARS-COV2 titer by RT-qPCR

| | | |
|---|---|---|
| 1 | MS2-F | TCCTGCTCAACTTCCTGTCGAG |
| | | (SEQ ID NO: 407) |
| 2 | MS2-R | CAGGTCAAACCTCCTAGGAATG |
| | | (SEQ ID NO: 408) |
| 3 | nCOV_N2 F | IDT, cat. No. 10006824 |
| 4 | nCOV_N2 R | IDT, cat. No. 10006825 |
| 5 | nCOV_N2 Probe | IDT, cat. No. 10006826 |

### OC43 Infectious Titer Via RT-qPCR

OC43 viral RNA was extracted from infected cell culture supernatants as described above (“Standard Methods”). The further steps of DNAse treatment and RT-qPCR were carried out as described above (“Standard Methods”). Virus titers were obtained by normalizing the viral RNA detected in the supernatant to the spiked in MS2 phage RNA.

TABLE 9
Primers used for OC43 titer by RT-qPCR

| | | |
|---|---|---|
| 1 | MS2-F | TCCTGCTCAACTTCCTGTCGAG |
| | | (SEQ ID NO: 407) |
| 2 | MS2-R | CAGGTCAAACCTCCTAGGAATG |
| | | (SEQ ID NO: 408) |
| 3 | OC43-QPCR-F | ATGTCAATACCCCGGCTGAC |
| | | (SEQ ID NO: 409) |
| 4 | OC43-QPCR-R | GGCTCTACTACGCGATCCTG |
| | | (SEQ ID NO: 410) |

### RSV Infectious Titer Via Focus-Forming Unit Assay

Virus supernatants were serially diluted in Opti-MEM. 24-well dishes of Hep-2 cells were infected with various serial dilutions of Respiratory Syncytial Virus stocks in 200 μL of Opti-MEM and incubated at 37° C. in 5% CO₂ for 2 h, gently rocking every 15 min. After removing the inoculum, the wells were overlaid with 2 mL of DMEM containing 2% FBS and 1% dissolved carboxymethylcellulose. The plates were incubated at 37° C. in a humidified 5% CO₂ incubator for 5 days without disturbing.

6 days post infection, the plaques in the monolayers were fixed using 1 mL of 4% PFA per well for a minimum of 1 h. Following this, the overlay plugs were removed by gentle tapping and water flow. Plates were then washed with PBS and treated with 0.5% (v/v) IGEPAL CA630 for 10 min and washed 2× with PBS. Subsequently, plates were incubated with PBSM to block for 1 h at room temperature on a shaker. After blocking, 1:1000 dilution of anti-RSV goat polyclonal antibody (Abcam, cat. No. ab20745) in PBSMT, was added and the plates incubated for 4 h at room temperature on a rocker. The plates were washed 3× with PBST and then incubated with a 1:1000 dilution of Peroxide-Conjugated Affinipure Rabbit anti-Goat Antibody (Jackson Immunoresearch, cat no. 305-035-003) in PBSMT and the plates incubated for 1 h at room temperature on a rocker.

Following incubation with the secondary antibody, the wells were washed 3× with PBST and the plaques developed using the TMB solution for blotting (Life Technologies Inc. Cat No. 002019). Viral titers were quantified as PFU/mL of tissue culture supernatant. For RSV bearing a fluorescent reporter gene-encoded into the viral genome, plaques were directly counted from the wells and imaged.

### HPIV Infectious Titer Via Focus-Forming Unit Assay or Plaque-Forming Unit Assay

Virus supernatants were serially diluted in Opti-MEM. 24-well dishes of LLC-MK2 cells or Hep2 cells were infected with various serial dilutions of human parainfluenza virus 1 (HPIV1) or human parainfluenza virus 3 (HPIV3) in 200 μL of Opti-MEM and incubated at 37° C. in 5% CO₂ for 2 h, gently rocking every 15 min. After removing the inoculum, the wells were overlaid with 2 mL of DMEM containing 2% FBS and 1% dissolved carboxymethylcellulose, or HPIV3 overlay media (MEM containing 1% Oxoid agar) for HPIV3 FFU assays. For the HPIV1 FFU assay, the cells were overlaid with HPIV1 overlay media (MEM containing 1% low melting point agarose and 1 mg/mL TPCK-Trypsin). The plates were incubated at 37° C. in a humidified 5% CO₂ incubator for 4 days (HPIV3) or 6 days (HPIV1) without disturbing.

After the 4 day (HPIV3) or 6 day incubation (HPIV1), the plates were fixed using 1 mL of 4% PFA per well for a minimum of 1 h. Following this, the overlay plugs were removed by gentle tapping and rinsing with dH₂O. Plates were then washed with PBS and treated with 0.5% (v/v) IGEPAL CA630 in DPBS for 10 min and washed 2× with PBS. Subsequently, plates were incubated with PBSM to block for 1 h at room temperature on a shaker. After blocking, 1:1000 dilution of anti-HPIV1 or anti-HPIV3 guinea-pig antiserum (NIAID, V321-511-558 or V323-501-558) in PBSMT, was added and the plates incubated for overnight at 4° C. on a rocker. The plates were washed 3× with PBST and then incubated with a 1:1000 dilution of Peroxide-Conjugated AffiniPure Goat Anti-Guinea Pig antibody (Jackson Immunoresearch, cat no. 106-035-003) in PBSMT and the plates incubated for 1 h at room temperature on a rocker.

Following incubation with the secondary antibody, the wells were washed 3× with PBST and the plaques developed using the TMB solution for blotting (Life Technologies Inc. Cat No. 002019). Viral titers were quantified as PFU/mL of tissue culture supernatant. Alternatively, for FFU assay with an agarose overlay, an additional overlay with 1% agarose and 0.25 mg/ml neutral red was added at 4- or 6-days post-infection. 48 hrs post addition of the second overlay the plaques in the monolayers were fixed using 1 mL of 4% PFA per well for a minimum of 1 h. Following this, the overlay plugs were removed by gentle tapping and water flow and plaques were visualized against the negative staining of the neutral red solution.

### HMPV Infectious Titer Via Focus-Forming Unit Assay

Virus supernatants were serially diluted in HMPV growth media (Opti-MEM containing 2% FBS, 100 U/mL penicillin, 100 μg/mL streptomycin, 2 mM glutamine, 50 μg/mL gentamycin, 2.5 μg/mL Amphotericin B, 100 μg/mL CaCl₂)). 24-well dishes of LLC-MK2 cells were infected with various serial dilutions of HMPV stocks in 200 μL of HMPV growth media and incubated at 37° C. in 5% CO₂ for 2 h, gently rocking every 15 min. After removing the inoculum, the wells were overlaid with HMPV overlay media containing up to 5 μg/ml TPCK-trypsin and 1% dissolved carboxymethylcellulose. The plates were incubated at 37° C. in a humidified 5% CO₂ incubator for 10 days without disturbing them.

10 days post infection, the plaques in the monolayers were fixed using 1 mL of 4% PFA per well for a minimum of 1 h. Following this, the overlay plugs were removed by gentle tapping and water flow. Plates were then washed with PBS and treated with 0.5% (v/v) IGEPAL CA630 for 10 min and washed 2× with PBS.

Subsequently, plates were incubated with PBSM to block for 1 h at room temperature on a shaker. After blocking, 1:1000 dilution of anti-HMPV mouse monoclonal antibody (GeneTex, Cat. No. GTX36792) in PBSMT, was added and the plates were incubated overnight at 4° C. on a rocker. The plates were washed 3× with PBST and then incubated with a 1:1000 dilution of Peroxide-Conjugated Affinipure Goat anti-Mouse Antibody (Jackson Immunoresearch, cat no. 115-035-003) in PBSMT and the plates incubated for 1 h at room temperature on a rocker. Following incubation with the secondary antibody, the wells were washed 3× with PBST and the plaques were developed using the TMB solution for blotting (Life Technologies Inc. Cat No. 002019). Viral titers were quantified as PFU/mL of tissue culture supernatant.

### Encephalomyocarditis Virus (EMCV) Titer Via Median Tissue Culture Infectious Dose Assay

Encephalomyocarditis Virus (EMCV) stocks or tissue culture supernatants from infected cells were diluted from 10-1× to 10-12× via ten-fold dilutions in D02. Human HeLa or mouse L929 cells plated in a confluent monolayer in 96-well dishes were infected with 100 μL of the above virus dilutions per well and incubated for 48 h at 37° C. in 5% CO₂. After 48 h or whenever clearance of wells was observed for lower dilutions of virus, the plates were fixed with 100 μL of 4% PFA per well for at least 1 hour.

The fixatives were removed by tipping the plate and washed with PBS. The plates were then stained with 0.1% crystal violet and the completely cleared wells recorded. TCID₅₀ concentration is calculated by the Spearman & Kärber algorithm as described in Hierholzer & Killington (1996), Virology Methods Manual, p. 374. Viral titer in TCID₅₀ was represented per mL of tissue culture supernatant.

### Example 1: Construction of Influenza Encrypted RNA Scaffolds and their DNA-Encoded Cassettes

DNA sequences were designed computationally and cloned by standard molecular biology methods. Source templates were typically obtained via amplification by PCR, restriction digest, synthetic oligonucleotides, or by custom synthesis of dsDNA via assembly of dsDNA from pools of overlapping and complementary oligonucleotides (e.g. gBlock or eBlock fragments available from Integrated DNA Technologies (IDT)). The identity of cloned DNA molecules was confirmed by a combination of restriction digests, Sanger sequencing (Genewiz), or next-generation sequencing (NGS) based on Illumina DNA sequencing technology.

The initial influenza A (IAV) antisense encrypted RNAs payloads were obtained by combining three independent sequence blocks (L, C, and R regions). ERNA-IAV-001-GDura was generated by concatenation of antis_5p_IAV (SEQ ID NO: 1), the reverse complement of GDura coding sequence (rcCDS_GDura, SEQ ID NO: 301), and antis_3p_IAV (SEQ ID NO: 18) in 5′ to 3′ order. ERNA-IAV-002-GDura was constructed similarly to ERNA-IAV-001-GDura, except antis_3p_IAV_enhanced (SEQ ID NO: 19) was substituted for antis_3p_IAV (SEQ ID NO: 18). Importantly, the three substitutions in antis_3p_IAV_enhanced (SEQ ID NO: 19) with respect to antis_3p_IAV (SEQ ID NO: 18) were anticipated to improve the performance of the encrypted RNA by increasing the level of the translated polypeptide of interest upon influenza infection (Neumann & Hobom, J. Gen Virology, 1995; DOI: 10.1099/0022-1317-76-7-1709).

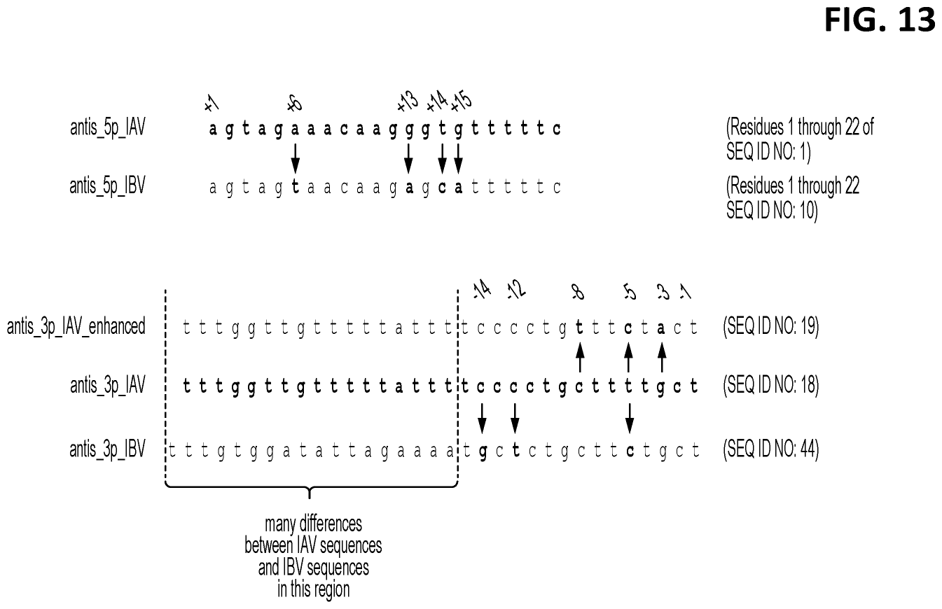

An encrypted RNA developed around an influenza B backbone was obtained similarly, but used L and R regions with greater similarity to influenza B. ERNA-IBV-001-GDura was generated by concatenation of antis_5p_IBV (SEQ ID NO: 10), the reverse complement of GDura coding sequence (rcCDS_GDura, SEQ ID NO: 301), and antis_3p_IBV (SEQ ID NO: 44). As shown in FIG. 13, the L and R regions of the influenza B encrypted RNAs are related but distinct from L and R regions of the influenza A encrypted RNAs.

TABLE 10
Sense and Antisense CDS for encoding within encrypted RNA

| | | | Antisense CDS | |
|---|---|---|---|---|
| | Sense CDS | | (reverse complement of Sense CDS) | |
| Protein | | SEQ | | SEQ |
| Sequence | | ID | | ID |
| Encoded | Sequence Name | NO | Sequence Name | NO |
| GDura | CDS_GDura | 273 | rcCDS_GDura | 301 |
| EGFP | CDS_EGFP | 274 | rcCDS_EGFP | 302 |
| Rluc8 | CDS_Rluc8 | 275 | rcCDS_Rluc8 | 303 |
| Human | CDS_human_IFN_beta_1_precursor | 278 | rcCDS_human_IFN_beta_1_precursor | 306 |
| IFN-β | | | | |
| Human | CDS_human_IFN_lambda_1_precursor | 279 | rcCDS_human_IFN_lambda_1_precursor | 307 |
| IFN- | | | | |
| lambda1 | | | | |
| Human | CDS_human_IFN_lambda_3_precursor | 280 | rcCDS_human_IFN_lambda_3_precursor | 308 |
| IFN- | | | | |
| lambda3 | | | | |
| Mouse | CDS_mouse_IFN_beta_1_precursor | 287 | rcCDS_mouse_IFN_beta_1_precursor | 315 |
| IFN-β | | | | |
| Mouse | CDS_mouse_IFN_lambda_2_precursor | 288 | rcCDS_mouse_IFN_lambda_2_precursor | 316 |
| IFN- | | | | |
| lambda2 | | | | |
| Mouse | CDS_mouse_IFN_lambda_3_precursor | 289 | rcCDS_mouse_IFN_lambda_3_precursor | 317 |
| IFN- | | | | |
| lambda3 | | | | |
| Syrian | CDS_Syrian- | 293 | rcCDS_Syrian- | 321 |
| Hamster | hamster_IFN_beta_precursor | | hamster_IFN_beta_precursor | |
| IFN-β | | | | |
| Ferret | CDS_ferret_IFN_beta_1_precursor | 295 | rcCDS_ferret_IFN_beta_1_precursor | 323 |
| IFN-β | | | | |

Introduction of Additional Protein Payloads into Influenza Encrypted RNA Scaffolds

The above 3 influenza antisense encrypted RNA scaffolds that can encode for Gdura were used as a basis to similarly encode alternative polypeptides of interest listed in Table 5 or Table 10 or others, including: EGFP, Rluc8, human interleukin 2 (IL-2), human interleukin 12 (IL-12), human IFN-β, human IFN-lambda1, human IFN-lambda3, mouse IFN-β, mouse IFN-lambda2, mouse IFN-lambda3, mouse interleukin 2 (IL-2), mouse interleukin 12 (IL-12), Syrian hamster IFN-β, or domestic ferret IFN-β.

For avoidance of ambiguity “hu_IFNB” means “human IFN-beta”; “m_IFNB” means “mouse IFN-beta”.

Similarly, three influenza antisense encrypted RNA scaffolds encoding alternative polypeptides of interest were created by replacing rcCDS_Gdura in ERNA-IAV-001-GDura, ERNA-IAV-002-GDura, and ERNA-IBV-001-GDura with another antisense CDS selected from Table 5 or Table 10.

Throughout the present disclosure, a systematic naming convention is used to describe encrypted RNA sequences, wherein “ERNA-IAV-X-Z” indicates that influenza A encrypted RNA Scaffold X encodes polypeptide of interest Z. Similarly, “ERNA-IBV-X-Z” indicates that influenza B encrypted RNA Scaffold X encodes polypeptide of interest Z.

For simplicity, when “Z” is “hu_IFNB”, the polypeptide of interest encoded in an encrypted RNA scaffold is human IFN-β. Similarly, “m_IFNB” means mouse IFN-β, and “ham_IFNB” means Syrian hamster IFN-β.

### DNA-Encoding of Influenza Encrypted RNA for In Vitro Production of Encrypted RNA

A DNA-encoded cassette for production of encrypted RNA via in vitro transcription was generated by cloning an influenza encrypted RNA scaffold in between a T7 promoter on the 5′ end (Promoter_T7_Core, SEQ ID NO: 331) and a convenient 3′ SapI restriction site (RE_SapI_3p, SEQ ID NO: 335) within the MCS of the pUC19 vector. For simplicity this construct is referred to as pAT201-X, where X is the influenza encrypted RNA scaffold. After linearization of a pAT201-series plasmid via SapI digestion, the linearized template can used to produce uncapped, 5′-triphosphorylated transcripts with standard in vitro transcription with four classic ribonucleotides (ATP, CTP, GTP, UTP) or 5′-capped transcripts (Cap 1) via co-transcriptional capping (e.g., by using CleanCap reagents, TriLink BioTechnologies).

To produce 5′-monophosphorylated influenza encrypted RNAs, alternative plasmid templates (pAT221-X, pAT222-X) were designed and constructed to incorporate a hammerhead ribozyme sequence within the 5′-end of the transcript preceding the influenza encrypted RNA scaffold sequence. Post-transcription, the hammerhead ribozyme divides the full length IVT product into a shorter 5′-triphosphorylated sequence and a full length 5′-monophosphorylated encrypted RNA, which can be separated by a variety of biochemical techniques (e.g., chromatography, selective precipitation).

Hammerhead ribozymes were developed with the following scheme to cleave the phosphodiester bond, which joins a 5′ hammerhead ribozyme sequence to a 3′ target sequence. Let Tq be target sequence q, H be a core hammerhead ribozyme, and Vq be the variant hammerhead sequence specific for Tq. The variant hammerhead sequence Vq is then set as the reverse complement of the target sequence Tq. To form a functional hammerhead ribozyme sequence, Vq and H were concatenated in 5′ to 3′ order and this concatemer (VqH) was appended immediately 5′ to the target sequence Tq so that the nucleotide immediately 3′ of the last nucleotide in H is the first nucleotide of Tq. Typically, the target sequence, Tq, is the first 12 nt of an encrypted RNA, and therefore Vq is the reverse complement of the 12 nt Tq sequence. H is typically a core hammerhead ribozyme sequence (UTR_5p_HHRz_core, SEQ ID NO: 340). As an example, the leading 12 nt of several influenza A antisense encrypted RNA are “agtagaaacaag”, so TIAV is “agtagaaacaag”. Therefore, an influenza A antisense hammerhead ribozyme sequence (UTR_5p_IAV_antis_HHRz, SEQ ID NO: 341) was constructed by concatenating the reverse complement of target sequence TIAV (VIAV=reverse_complement (TIAV)=“cttgtttctact”) to a core hammerhead ribozyme sequence (UTR_5p_HHRz_core, SEQ ID NO: 340).

Plasmid template family pAT221-X was created similarly to pAT201-X by concatenation of an alternative T7 promoter (Promoter_T7_AT, SEQ ID NO: 332), influenza A antisense hammerhead ribozyme sequence (UTR_5p_IAV_antis_HHRz, SEQ ID NO: 341), influenza A antisense encrypted RNA, and convenient 3′ SapI restriction site (RE_SapI_3p, SEQ ID NO: 335). This construct is referred to as pAT221-X, where X is the influenza A antisense encrypted RNA scaffold.

Plasmid template family pAT222-X was created analogously to pAT221-X by concatenation of an alternative T7 promoter (Promoter_T7_AT, SEQ ID NO: 332), influenza B antisense hammerhead ribozyme sequence (UTR_5p_IBV_antis_HHRz, SEQ ID NO: 342), influenza B antisense encrypted RNA, and convenient 3′ SapI restriction site (RE_SapI_3p, SEQ ID NO: 335). This construct is referred to as pAT222-X, where X is the influenza B antisense encrypted RNA scaffold.

### DNA-Encoding of Influenza Encrypted RNA for Intracellular Production of Encrypted RNA

A DNA-encoded influenza encrypted RNA cassette for use in primate cells (human or non-human primates) was generated by cloning an influenza encrypted RNA cassette in between a human PolI promoter sequence (Promoter_PolI_human, SEQ ID NO: 353) and a mouse PolI terminator sequence (Terminator_PolI_mouse, SEQ ID NO: 355) within a commercial cloning vector kit (pCR-BluntII-TOPO) (Thermo Fisher Scientific, cat. No. 450245). For brevity, this vector is referred to as pAT001. A similar version incorporating an optimized terminator sequence (Terminator_PolI_mouse_enhanced, SEQ ID NO: 356) was prepared similarly and is described as pAT002. A related vector, pAT004, was prepared similarly to pAT001 and pAT002, but utilized another terminator sequence (Terminator_PolI_mouse_enhanced_4, SEQ ID NO: 357). In all systems, transfection of a suitable human or non-human primate-derived cell line (e.g., A549, 293T) with pAT001, pAT002, or pAT004 encoding an encrypted RNA construct leads to the PolI-driven production of a single-stranded, 5′-triphosphorylated encrypted RNA within the nucleus.

Using this naming convention, the following five DNA-encoded constructs were generated: pAT001-ERNA-IAV-001-GDura, pAT001-ERNA-IAV-002-GDura, pAT002-ERNA-IAV-001-GDura, pAT002-ERNA-IAV-002-GDura, and pAT002-ERNA-IBV-001-GDura.

In addition to the constructs explicitly listed, it is clear that any of the influenza encrypted RNA constructs could be generated similarly by inserting the influenza antisense encrypted RNA construct into the same position within pAT001 or pAT002. To simplify nomenclature, pAT002-ERNA-IAV-001-GDura would indicate that ERNA-IAV-001-GDura was cloned into the central position within pAT002 for proper expression.

As PolI promoters may have limited biological activity outside of their host species, additional derivatives of pAT002 were developed that incorporate an alternative PolI promoter for use in a different species (e.g. a PolI promoter suitable for mouse cells), but otherwise retain the same genetic elements of pAT002. For example, construct pAT003 was developed, which used the sequence elements of pAT002, but substituted a mouse PolI promoter sequence (Promoter_PolI_mouse, SEQ ID NO: 354) for the previous human PolI promoter sequence (Promoter_PolI_human, SEQ ID NO: 353). pAT003 was anticipated to be used primarily in live mice and in mouse cells due to the general loss-of-function of PolI promoters when used in cells derived from a host species substantially unrelated to the origin species of the PolI promoter. The restriction of the activity of a particular PolI promoter to some species stems from the potential incompatibility of PolI transcription factors across different species (Eberhad & Grummt, DNA Cell Bio. (1996); DOI: 10.1089/dna.1996.15.167).

Demonstration of Enhanced 3′ End Formation with Improved Terminator Sequences

An RNA transcript of similar size to ERNA-IAV-001-GDura and ERNA-IAV-002-GDura and incorporating the same three terminal 3′ nucleotides was cloned identically into pAT001 (Terminator_PolI_mouse, SEQ ID NO: 355), pAT002 (Terminator_PolI_mouse_enhanced, SEQ ID NO: 356), pAT004 (Terminator_PolI_enhanced_d4, SEQ ID NO: 357) to create pAT001-GCU, pAT002-GCU, and pAT004-GCU.

To compare the differences in 3′ transcript termini sequences conferred by each of these promoter sequences, human 293T cells were transfected with each of these plasmids encoding the same transcript. Cellular RNA was isolated at approximately 24 h, and the 3′ ends of the transcripts profiled by 3′-RACE (Rapid Amplification of cDNA Ends) via molecular cloning and Sanger sequencing.

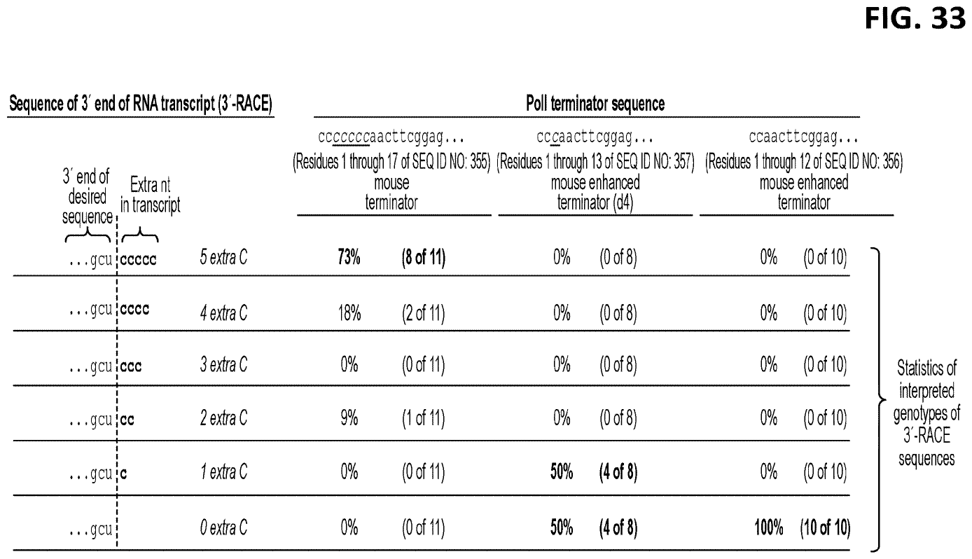

FIG. 33 shows that transcripts produced by using a human PolI promoter sequence in concert with conventional PolI terminator sequence within pAT001 (Terminator_PolI_mouse, SEQ ID NO: 355), generally have 5 additional cytosine nucleotides at their 3′ end (approximately 75% of 11 examined sequences), and seldom have the exact 3′ terminus desired (0 of 11 clones sequenced). This was a surprising finding, as the Terminator_PolI_mouse sequence is commonly utilized in bi-directional influenza reverse genetics systems. An incorrect addition or insertion of additional nucleotides at the termini of viral RNA would be expected to hamper the efficiency of viral rescue and inhibit virus replication. Notably, Terminator_PolI_mouse (SEQ ID NO: 355) is a common terminator sequence in influenza reverse genetics systems (see, for example, FIG. 2 of Neumann et al., Methods Mol Biol 2012, DOI: 10.1007/978-1-61779-621-0_12).

In contrast, removal of 5 deoxycytidine nucleotides from the 5′ end of Terminator_PolI_mouse (SEQ ID NO: 355) to create Terminator_PolI_mouse_enhanced (SEQ ID NO: 356) was sufficient to enable error-free and faithful reproduction of the desired 3′ end of the RNA transcript, as 100% of sequenced clones (10 of 10) contained no undesired additional terminal nucleotides. Deletion of only 4 deoxycytidine nucleotides from the 5′ end of Terminator_PolI_mouse (SEQ ID NO: 355) to create Terminator_PolI_mouse_enhanced_d4 (SEQ ID NO: 357) was also helpful in allowing faithful reproduction of the RNA transcript, as 50% of sequenced clones (4 of 8) contained no additional terminal nucleotides, while the remaining 50% (4 of 8) contained only one additional C nucleotide.

### Example 2: Activation of Encrypted RNAs by Virus Infection or RdRPs

FIG. 4A shows a simplified schematic of an experiment to test the activation of an encrypted RNA after viral infection of treated cells.

More specifically, human 293T cells (4×10⁴ cells) were transfected with a mixture of plasmids comprising 90 ng of pmaxGFP (Lonza) and 10 ng of pAT001- or pAT002-based encrypted RNAs encoding GDura as a polypeptide of interest. Sixteen hours later, cells were either mock-infected (MOI-0) or infected with influenza A (A/PR8) at an MOI ranging from 0.003 to 10 in 0.5 log intervals. At 24 hours after infection, the GDura containing supernatant was harvested and the amount of secreted luciferase measured by plate reader assay as described above.

As shown in FIG. 4B, cells that were either untreated with a DNA-encoded encrypted RNA or treated with a DNA-encoded encrypted RNA but uninfected showed approximately equivalent and low levels of translated luciferase payload. In contrast, cells treated with a DNA-encoded encrypted RNA and then infected with influenza showed an influenza MOI-dependent level of translation and secretion of the luciferase (GDura) polypeptide of interest. Importantly, modifying the 3′-flank of encrypted RNA v1 (pAT001-ERNA-IAV-001-GDura) to encrypted RNA v2 (pAT001-ERNA-IAV-002-GDura), resulted in an approximately 100× increase in secreted luciferase protein. Further modification of encrypted RNA v2 to encrypted RNA v3 (pAT002-ERNA-IAV-002-GDura) increased the activation of the DNA-encoded encrypted RNA for influenza infections at low MOI.

### Example 3: A Target-Specific Translation Activator can Activate Encrypted RNA in the Absence of Viral Infection

Pools of plasmid DNA were prepared and used for transfection. A 1000 ng DNA pool termed “Pola” comprised of 200 ng of each plasmid producing the influenza PB2, PB1, PA, and NP proteins to reconstitute a complete influenza A/PR8 polymerase complex supplemented by nucleocapsid protein; 100 ng of pAT001-ERNA-IAV-002-GDura; 100 ng of pmaxGFP (Lonza) plasmid. Pool “PolA-PB2” was prepared similarly, but omitted the 200 ng of PB2 production plasmid and increased the pmaxGFP mass to 300 ng to maintain the total mass of DNA at 1000 ng. Likewise, pool “Pola-PB1” omitted only PB1 with respect to pool PolA; pool “PolA-PA” omitted only PA with respect to pool PolA, and pool “PolA-ERNA” omitted pAT001-ERNA-IAV-GDura with respect to pool PolA. In all cases the total mass of DNA was brought to 1000 ng by adding supplemental pmaxGFP plasmid, if necessary. Pool “no TA” was prepared by combining 100 ng of pAT001-ERNA-IAV-GDura with 900 ng of pmaxGFP.

Human 293T (7.5×10⁵ cells per well of 12-well plate) were transfected with one of the above six pools or left untransfected and the amount of the translated polypeptide of interest (secreted GDura luciferase) was quantified at 24 hours post-transfection as described above. In this Example, if the level of GDura increased more than 10× relative to untransfected cells, then the polypeptide of interest (GDura) was said to have increased translation.

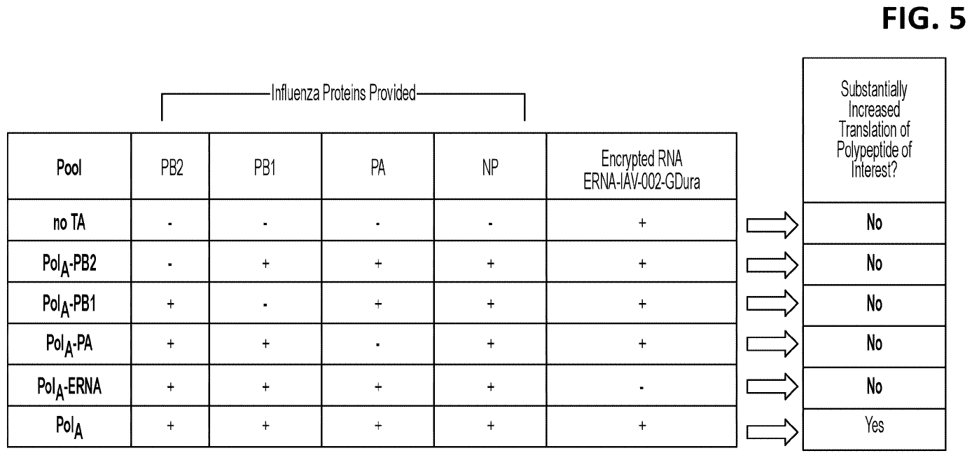

As shown in FIG. 5, co-transfection of a complete polymerase complex and accessory proteins (together, a translation activator) with an encrypted RNA (pAT001-ERNA-IAV-002-GDura) can lead to increased translation of the polypeptide of interest. In FIG. 5, co-transfection of Pola with the influenza encrypted RNA encoding GDura as the polypeptide of interest results in increased translation of GDura. Thus, encrypted RNAs can be activated by a target-specific translation activator outside of the context of a viral infection. Further, if any of the elements of the influenza polymerase trimeric complex (PB1, PB2, or PA) were removed from PolA, the influenzae encrypted RNA was not substantially activated.

### Example 4: Production of Uncapped RNA from a Linear PCR Template

DNA Template Generation with Forward T7 and Reverse Poly(A) Primers

A linear PCR template was generated in a 3-step PCR using PrimeStar MAX DNA polymerase (Takara, cat. No. R045B). Forward and reverse primers were obtained as custom DNA oligonucleotides from a commercial supplier (GENEWIZ).

In order to both amplify the linear DNA template and add additional sequences necessary for efficient IVT, the forward primer was designed to add the T7 promoter sequence to the 5′ end of the amplicon while the reverse primer was designed to add a sequence of 120 T bases 3′ of the amplified region of the template DNA. Representative example sequences used for generation of T7-Apt17-containing linear PCR templates are shown in Table 11 below:

TABLE 11

| | |
|---|---|
| T7 forward primer | gcaacatatgTAATACGACTCACTATAG |
| | GGACTCACTATTTGTTTTCGCGCCCAG |
| | (SEQ ID NO: 411) |
| 120T reverse primer | TTTTTTTTTTTTTTTTTTTTTTTTTTTT |
| | TTTTTTTTTTTTTTTTTTTTTTTTTTTT |
| | TTTTTTTTTTTTTTTTTTTTTTTTTTTT |
| | TTTTTTTTTTTTTTTTTTTTTTTTTTTT |
| | TTTTTTTTCTCTTTGCCGTATCCCATCT |
| | TAG |
| | (SEQ ID NO: 412) |

A typical reaction was formed by combining:
1 μL (20 ng/μL) plasmid DNA template
1.5 μL forward primer
1.5 μL reverse primer
5 μL GC enhancer (New England Biolabs, cat. No. B9027S)
3.5 μL nuclease-free water (New England Biolabs, cat. No. B1500S)
12.5 μL PrimeStar MAX DNA polymerase 2× Master Mix
Total volume 25 μL

To obtain a sufficient quantity of PCR template, 3 to 4 PCR reactions were run in parallel and subsequently pooled. PCR product length was confirmed by gel electrophoresis in 1% agarose (VWR, cat. No. 0710) and then was purified using Mag-Bind TotalPure NGS bead (Omega Bio-Tek, cat. No. M1378-01) according to manufacturer's protocol and the final product was eluted in 20 μL NFW. The concentration of DNA was determined by measuring the absorbance at 260 nm using a UV/Vis spectrophotometer (ThermoFisher NanoDrop 2000) and the concentration calculated from the absorbance using a nominal extinction coefficient of 50 μg/mL/cm.

In Vitro Transcription (IVT) with a Non-Thermostable T7 RNA Polymerase

IVT reactions were run using HiScribe High Yield RNA synthesis kit (New England Biolabs, cat. No. E2040S). A standard reaction setup was:

| | |
|---|---|
| Nuclease-free water | 11 μL − X |
| ATP, 100 mM | 1 μL |
| UTP, 100 mM | 1 μL |
| CTP, 100 mM | 1 μL |
| GTP, 100 mM | 1 μL |
| 10× Reaction buffer | 2 μL |
| DNA, 500 ng | X μL |
| T7 Enzyme mix | 3 μL |
| Total | 20 μL |

All components of the reaction were combined in 1.5 mL DNA LoBind tube (Eppendorf, cat. No. 022431021) and incubated for 2.5-3.0 h at 37° C. in a dry air incubator (ThermoFisher). After the incubation, 2 μL of DNAse I (2 U/μL) (New England Biolabs, cat. No. M0303) was added and the reaction was incubated for an additional 15 min at 37° C.

### In Vitro Transcription at Elevated Temperature Using a Thermostable RNA Polymerase

Alternatively, RNA was produced via IVT using a variant of E. coli T7 phage RNA polymerase that is more stable than a “wildtype” reference T7 RNA polymerase (T7 RNAP) in reactions at elevated temperatures (e.g, 40° C., 45° C., 48° C., 50° C., 52° C., 54° C., 56° C.). These “thermostable T7 RNA polymerases” or “thermostable T7 RNAP” are well-known and may be commercially obtained (e.g., Hi-T7 RNA Polymerase from New England Biolabs). In vitro transcription reactions using thermostable T7 RNA Polymerases and allow in vitro transcription to occur at elevated temperatures above 37° C., which can lower the immunogenicity of produced RNA, e.g., by reducing the formation of 3′ extended run-off transcripts (Wu et al., RNA (2020): DOI: 10.1261/rna.073858.119).

To prepare RNA via IVT using a thermostable T7 RNAP, IVT reactions were prepared from individual components or from a kit (New England Biolabs). Most reagents were obtained from New England Biolabs: NFW (New England Biolabs, cat. No. B1500); ATP, CTP, GTP, UTP, all at 100 mM (New England Biolabs, cat. No. N0450); E. coli inorganic pyrophosphatase ((New England Biolabs, cat. No. M0361); RNAse Inhibitor, murine (New England Biolabs, cat. No. M0314), Hi-T7 RNA Polymerase (New England Biolabs, M0658S).

The composition of a typical 40 μL IVT reaction using a thermostable T7 RNAP was:

| | |
|---|---|
| Nuclease-free water (New England Biolabs, cat. No. | 22 μL − X |
| B1500) | |
| ATP, 100 mM (New England Biolabs, cat. No. N0450) | 2 μL |
| UTP, 100 mM (New England Biolabs, cat. No. N0450) | 2 μL |
| CTP, 100 mM (New England Biolabs, cat. No. N0450) | 2 μL |
| GTP, 100 mM (New England Biolabs, cat. No. N0450) | 2 μL |
| 10× ATI20 Reaction buffer | 4 μL |
| E. coli IPP (New England Biolabs, cat. No. M0361) | 0.8 μL |
| RNAse Inhibitor, murine (New England Biolabs, cat. | 2 μL |
| No. M0314) | |
| DNA template, 1 μg | X μL |
| Hi-T7 Polymerase (New England Biolabs, M0658S) | 3.2 μL |
| Total | 40 μL |

Composition of ATI20:
10 mM Tris-HCl, pH 8 (VWR, cat. No. E199-500ML)
20 mM MgCl₂ (Quality Biological, cat. No. 351-033-721)
2 mM spermidine (ThermoFisher, cat. No. A19096-06)
2 mM dithiothreitol (Acros, cat. No. AC16568-0050)

| | |
|---|---|
| NFW | 8.2 μL |
| 10× Reaction Buffer | 2.0 μL |
| ATP, 100 mM | 1.0 μL |
| UTP, 100 mM | 1.0 μL |
| CTP, 100 mM | 1.0 μL |
| GTP, 100 mM | 1.0 μL |
| CleanCap, 100 mM | 0.8 μL |
| DNA, 250 ng/μL | 2.0 μL |
| T7 Enzyme mix | 3.0 μL |
| Final Volume | 20.0 μL |

IVT Reactions were typically incubated for 3 h at 50° C. before purification. In some reactions, the concentration of magnesium in ATI20 was increased or decreased.

### Reaction Clean-Up

The IVT-synthesized RNA was purified from the completed reaction with Monarch RNA CleanUp Kit (New England Biolabs, cat. No. T2050L) according to manufacturer's protocol.

### Example 5: Production of Capped RNA from a Linear PCR Template

DNA Template Generation with Forward T7 and Reverse Poly(A)

The same protocol was used as described in Example 4.

### In Vitro Transcription Using a Non-Thermostable T7 RNA Polymerase

IVT reaction was run using HiScribe High Yield RNA synthesis kit (New England Biolabs, cat. No. E2040S) and CleanCap AG (TriLink, cat. No. N-7113) or CleanCap AU (Trilink, cat. No. N-7114) reagents depending on the second base of template after transcription start. Standard reactions were setup using the following recipe:

| | |
|---|---|
| NFW | 8.2 μL |
| 10× Reaction Buffer | 2.0 μL |
| ATP, 100 mM | 1.0 μL |
| UTP, 100 mM | 1.0 μL |
| CTP, 100 mM | 1.0 μL |
| GTP, 100 mM | 1.0 μL |
| CleanCap, 100 mM | 0.8 μL |
| DNA, 250 ng/μL | 2.0 μL |
| T7 Enzyme mix | 3.0 μL |
| Final Volume | 20.0 μL |

Incubation and following treatment are the same as in Example 4.

### In Vitro Transcription at Elevated Temperature Using a Thermostable T7 RNA Polymerase

In vitro transcriptions using a thermostable T7 RNA Polymerase were prepared similarly to that described in Example 4, except a typical reaction included CleanCap (TriLink). A typical reaction was prepared from individual components, explained in Example 4:

| | |
|---|---|
| Nuclease-free water (New England Biolabs, cat. | 20.4 μL − X |
| No. B1500) | |
| ATP, 100 mM (New England Biolabs, cat. No. N0450) | 2 μL |
| UTP, 100 mM (New England Biolabs, cat. No. N0450) | 2 μL |
| CTP, 100 mM (New England Biolabs, cat. No. N0450) | 2 μL |
| GTP, 100 mM (New England Biolabs, cat. No. N0450) | 2 μL |
| CleanCap, 100mM | 1.6 μL |
| 10× ATI20 Reaction buffer | 4 μL |
| E. coli IPP (New England Biolabs, cat. No. M0361) | 0.8 μL |
| RNAse Inhibitor, murine (New England Biolabs, cat. | 2 μL |
| No. M0314) | |
| DNA, 1 μg | X μL |
| Hi-T7 Polymerase (New England Biolabs, M0658S) | 3.2 μL |
| Total | 40 μL |

### Reaction Clean-Up

Reaction clean-up was performed as described in Example 4.

### Example 6: Production of Capped RNA or Uncapped RNA from a Plasmid Template

### IVT Plasmid Linearization

IVT plasmid DNA templates were linearized by treatment with type IIS restriction endonucleases: SapI (New England Biolabs, cat. No. R0569L) or BspQI (New England Biolabs, cat. No. R0712L) or BbsI (New England Biolabs, cat. No. R0539L). Restriction digests were conducted per the manufacturer's recommend protocols. Treatment of a designated IVT plasmid template with an above type IIS restriction endonuclease cuts the plasmid at a single site, generally cutting asymmetrically after the final adenosine in the poly(A) tail via the unique Type IIS restriction site positioned immediately 3′ of the poly(A) tail in the correct strand orientation, to allow plasmid cleavage without the addition of non-A nucleotides at 3′ end of the resulting poly(A) tail. For some RNA transcripts, a poly(A) tail was not included by design and the type IIS sequence was therefore positioned 3′ of the transcript template to generate the desired RNA.

### In Vitro Transcription & Co-Transcriptional Capping

Capped RNA was produced using approaches detailed in Example 4 (with thermostable T7 RNA Polymerase or non-thermostable T7 RNA Polymerase), but with the inclusion of a 5′-Cap analog in the IVT buffer using the manufacturer's recommended conditions (CleanCap product, TriLink Biotechnologies).

A standard IVT reaction buffer consists of:

TABLE 12

| Component | Final concentration | Source |
|---|---|---|
| Tris, pH 8.0 | 10 mM | VWR, E199-500ML |
| MgCl₂ | 16 mM | Quality Biological, 351-033-721 |
| spermidine | 2 mM | Thermo, A19096-06 |
| DTT (1,4-dithiothreitol) | 2 mM | Acros AC16568-0050 |
| ATP | 0-5 mM | New England Biolabs, N0450L |
| CTP | 0-5 mM | New England Biolabs, N0450L |
| GTP | 0-5 mM | New England Biolabs, N0450L |
| UTP | 0-5 mM | New England Biolabs, N0450L |
| N⁶-methyladenosine-5′- | 0-5 mM | (Trilink, N-1013), |
| triphosphate (m⁶A) | | |
| N1-methylpseudouridine-5′- | | (Trilink, N-1081) |
| triphosphate (m¹Ψ) | | |
| pseudouridine-5′-triphosphate | | |
| (Ψ) (Trilink, N-1019) | | |
| 5-methylcytidine-5′-triphosphate | | (Trilink, N-1014) |
| (m¹C) | | |
| DNA Template | 50 ng/μL | |
| Murine RNAse inhibitor | 2 U/μL | New England Biolabs, M0314 |
| Thermostable inorganic | 0.1 U/μL | New England Biolabs, M0296 |
| pyrophosphatase | | |
| M5 T7 RNA pol | 200 nM | |
| CleanCap AG | 0.4 mM | Trilink, N-7113 |
| CleanCap AU | 0.4 mM | Trilink, N-7114 |

When IVT was performed using a thermostable T7 RNA polymerase, reactions were often assembled on ice, initiated with the addition of a thermostable T7 RNA polymerase and incubated for 3 h at 50° C. in a thermocycler. After 3 hours, DNAse I (RNAse-free) (New England Biolabs, cat. No. M0303) was added at 0.1 U/μL of reaction and incubated at 37° C. for an additional 30 min. The resulting full-length mRNA was isolated from the starting materials using a Monarch RNA Cleanup Kit (New England Biolabs, cat. No. T2050) eluted in NFW per the manufacturer's instructions The concentration of RNA was determined by measuring the absorbance at 260 nm using a UV/Vis spectrophotometer (ThermoFisher, NanoDrop 2000) with a nominal extinction coefficient of 40 μg/mL/cm. The size and purity of IVT reactions was assessed by running the product on a denaturing agarose gel (MOPS supplemented with 2% formaldehyde) and visualization with ethidium bromide or SYBR Gold (Thermo Fisher Scientific, cat. No. S11494).

### Example 7: Production of Circular Encrypted RNA from a Plasmid Template

### Synthesis and Circularization

Circular encrypted RNA was synthesized via IVT using a thermostable or conventional T7 RNA polymerase, as described above in Example 6. DNA-encoded cassettes for preparation of linear encrypted RNA were converted to DNA-encoded cassettes for preparation of circular encrypted RNA by the addition of a 5′ flanking sequence (UTR_5p_circ_RNA, SEQ ID NO: 349) to the 5′ end of the original RNA scaffold sequence and addition of a 3′ flanking sequence (UTR_3p_circ_RNA, SEQ ID NO: 350) to the 3′ end of the original RNA scaffold sequence. The addition of both sequences allows for the RNA formed via in vitro transcription to be circularized via the action of the encoded self-splicing intron (Wesselhoeft et al., Mol. Cell 2019; DOI: 10.1016/j.molcel.2019.02.015).

Briefly, IVT reactions of SapI-treated circular encrypted RNA plasmid templates were prepared and incubated as described above in Example 6 (typically 3 h at 37° C.). After 3 h, the reaction was subsequently treated by adding DNAse I (New England Biolabs, cat. No. M0303L) to a final concentration of 0.2 U/μL and incubating for 15 min at 37° C. The resulting RNA was purified from the completed reaction with Monarch RNA CleanUp Kit (New England Biolabs, cat. No. T2050L) according to manufacturer's protocol and eluted in NFW.

For circularization of IVT products, GTP was added to a final concentration of 2 mM, and then reactions were heated at 55° C. for 15 min. RNA was then column purified with Monarch RNA CleanUp Kit. To enrich for circular encrypted RNA, 40 μg of RNA was diluted in water (88 AL final volume) and then heated at 65° C. for 3 min and cooled on ice for 3 min. Next, 20 U RNAse R (Lucigen, cat. No. RNR07250) and 10 μL of 10×RNAse R buffer (Lucigen) were added, and the reaction was incubated at 37° C. for 8 min; an additional 20 U RNAse R was added, and reaction was incubated for an additional 8 minutes. A final purification of the RNAse R-digested RNA was performed with Monarch RNA CleanUp Kit (New England Biolabs, cat. No. T2050L) prior to HPLC purification.

### HPLC Purification

HPLC fractionation of circular encrypted RNA was achieved using a reversed-phase column (Agilent, Zorbax Extend 300 C18, 4.6 mm×150 mm, 3.5 micron) operated at 0.5 mL/min via a ThermoScientific uHPLC3000a. Both mobile and solvent phases were prepared with NFW.

Mobile phase A consisted of Solution A (0.1 M triethylammonium acetate (TEAA), pH 7.5 in NFW). Solvent phase B consisted of Solution B (0.1 M TEAA pH 7.5 in 25% (v/v) acetonitrile in NFW).

TABLE 13
Chromatograph programming

| Time (min) | Solution A (%) | Solution B (%) | Elution |
|---|---|---|---|
| 0-1 | 55 → 49 | 45 → 51 | linear gradient |
| 1-9 | 49 → 40 | 51 → 60 | linear gradient |
| 9-10.5 | 40 → 0 | 60 → 100 | linear gradient |
| 10.5-11 | 0 → 55 | 100 → 45 | step gradient |
| 11-18 | 55 | 45 | re-equilibrium |

Fractions were collected manually and then concentrated using an Amicon Ultra-0.5 centrifugal filter unit (Millipore, cat. No. UFC510024). Concentration of RNA was determined by measuring the absorbance at 260 nm using a UV/Vis spectrophotometer (ThermoFisher NanoDrop 2000) and a nominal extinction coefficient of 40 μg/mL/cm.

### Example 8: Complexation of Encrypted RNA into LMAX-LNPs Via Commercial Lipid Nanoparticle Product

RNA was formulated into lipid nanoparticles (LNPs) suitable for transient transfection via a complexation with a commercially available product, Lipofectamine MessengerMAX Transfection Reagent (ThermoFisher, cat. No. LMRNA008), per the manufacturer's instructions. As the manufacturer's instructions provide some latitude in use of the lipid reagent, a representative example is provided here to clarify how the reagent was frequently used.

For transfection of cells at the 24 well-plate scale with up to 250 ng of RNA, the following procedure was used. Solution LipoA was prepared by adding 1.5 μL of Lipofectamine MessengerMAX Transfection Reagent (ThermoFisher, cat. No. LMRNA008) to 25 μL of Opti-MEM. Solution LipoB was prepared by adding 250 ng of RNA to 50 μL Opti-MEM. Solutions LipoA and LipoB were separately, but contemporaneously, incubated for a minimum of 10 min at room temperature (20-25° C.). After the 10 min parallel incubations, the entire volume of Solution LipoB was added to Solution LipoA and the resulting mixture, termed Solution LipoAB, briefly mixed and then further incubated for 5-15 min at room temperature (20-25° C.). After the 5-15 min incubation, Solution LipoAB was added dropwise to cells.

In order to distinguish RNA formulated into LNPs via the Lipofectamine MessengerMAX Transfection Reagent (LMAX) from other potential LNP formulations (e.g. the alternative LNP formulations described in Example 9), RNA formulated into LNPs via LMAX are referred to as: LMAX-LNP formulated RNA, LMAX-LNP encapsulated RNA, or equivalents.

### Example 9: Preparation of Alternative LNP-Formulated Preparations of RNA or Encrypted RNA

In addition to the LMAX-formulated RNAs described in Example 8, LNP-formulated encrypted RNAs were prepared using alternative lipid nanoparticle compositions and methods.

A skilled practioner in the art can readily formulate RNA molecules into a variety of LNPs, including LNPs suitable for the delivery of encapsulated RNA to animal and human subjects, such as the LNPs used to deliver FDA-approved small-interfering RNA therapeutics or COVID-19 mRNA vaccines to human subjects (see, for example, Buschmann et al., Nanomaterial Delivery Systems for mRNA vaccines, Vaccines 2021; DOI: 10.3390/vaccines9010065).

At the simplest level, lipid transfection reagents used to prepare LNP-formulated RNA can be obtained commercially and LNP-formulated RNAs can be prepared per the manufacturer's directions. Additional examples of lipid transfection reagents include: FUGENE HD (Promega), LipoD293 (SignaGen Laboratories), LipoJet (SignaGen Laboratories), MegaFectin (Qbiogene), or TransFectin (Bio-Rad).

LNP-formulated RNA may also be formed by acquiring one or more readily available lipid components from a commercial supplier (e.g., Cayman Chemical Company, Polysciences, or Avanti Polar Lipids) and preparing LNPs using a published or readily available method. Lipid nanoparticles may be formed from cationic lipids such as: DOTAP MS (N-[1-(2,3-Dioleoyloxy) propyl]-N,N,N-trimethylammonium methyl-sulfate); DOTMA (N-[1-(2,3-Dioleoyloxy) propyl]-N,N,N-trimethylammonium chloride); DODMA (1,2-dioleyloxy-3-dimethylaminopropane); DODAP (1,2-dioleoyl-3-dimethylammonium propane); DSTAP Chloride (1,2-distearoyl-3-trimethylammonium-propane chloride); or DOSPA Hydrochloride (N-(2-(2,5-Bis((3-aminopropyl)amino) pentanamido)ethyl)-N,N-dimethyl-2,3-bis((Z)-octadec-9-en-1-yloxy) propan-1-aminium chloride tetrahydrochloride)). LNPs may be formed by a variety of methods, including: thin film hydration (Jain et al., Mol Pharmaceuticals (2013); DOI: 10.1021/mp400036w), sonication, or extrusion (Lapinski et al., Langmuir (2007); DOI: 10.1021/la7020963).

LNPs that are suitable for RNA delivery in vitro and in vivo are now common (Hou et al, Nat Rev Mater (2021); DOI: 10.1038/s41578-021-00358-0), with pharmaceutical grade lipid components and preparation methods available from a variety of sources (e.g., Precision NanoSystems). LNPs are typically comprised of four lipid or lipid-like components: (i) a cholesterol or cholesterol derivative; (ii) a cationic lipid, sometimes called an ionizable lipid; (iii) a structural lipid, sometimes called a phospholipid; and (iv) a PEG lipid, sometimes called a PEGylated lipid, which is a polyethylene glycol (PEG) functionalized lipid used to stabilize the particle and improve product stability and pharmacokinetic properties due to surfactant properties (e.g., see Hou et al, Nat Rev Mater (2021); DOI: 10.1038/s41578-021-00358-0). Example cholesterol or cholesterol derivatives include: cholesterol ((3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-ol); cholesteryl arachidonate ([(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl](5Z,8Z,11Z,14Z)-icosa-5,8,11,14-tetraenoate); cholesteryl linoleate ([(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl](9Z,12Z)-octadeca-9,12-dienoate); or beta-sitosterol ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol). Example cationic lipids or ionizable lipids include: D-Lin-MC3-DMA or “MC3” ((6Z,9Z,28Z,31Z)-heptatriaconta-6,9,28,31-tetraen-19-yl 4-(dimethylamino)butanoate); DOTAP (1,2-dioleoyl-3-trimethylammonium-propane); c12-200 (1,1′-((2-(4-(2-((2-(bis(2-hydroxydodecyl)amino)ethyl)(2-hydroxydodecyl)amino)ethyl) piperazin-1-yl)ethyl)azanediyl)bis(dodecan-2-ol)); cKK-E12 (3,6-bis(4-(bis(2-hydroxydodecyl)amino)butyl) piperazine-2,5-dione); SM-102 (Heptadecan-9-yl 8-((2-hydroxyethyl)(6-oxo-6-(undecyloxy)hexyl)amino)octanoate); ALC-0315 (((4-Hydroxybutyl) azanediyl)bis(hexane-6,1-diyl)bis(2-hexyldecanoate)); or others detailed in (Han et al., Nat Comm (2021); DOI: 10.1038/s41467-021-27493-0 or Hou et al, Nat Rev Mater (2021); DOI: 10.1038/s41578-021-00358-0). Representative PEG lipids include: DMG-PEG 2000 (1,2-dimyristoyl-rac-glycero-3-methoxypolyethylene glycol-2000); or ALC-0159 (α-[2-(ditetradecylamino)-2-oxoethyl]-ω-methoxy-poly(oxy-1,2-ethanediyl). Representative structural lipid components include: DSPC (1,2-distearoyl-sn-glycero-3-phosphocholine); DOPE (1,2-dioleoyl-sn-glycero-3-phosphoethanolamine; or DPPC (1,2-dipalmitoyl-sn-glycero-3-phosphocholine).

To prepare some LNPs, form a lipid mix by combining one or more lipid-like compounds (e.g., a cholesterol, a cationic lipid, a structural lipid, or a PEG lipid) in an organic solvent (such as ethanol or chloroform). Subsequently, combine the lipid mix (in a suitable organic solvent) with an aqueous solution of RNA in a low pH buffer via rapid mixing or a microfluidic device (e.g., WO2017049258A2, U.S. Pat. Nos. 11,357,856, 1,168,051) to form a lipid nanoparticle emulsion encapsulating nucleic acids. After the emulsion is formed, carefully change the aqueous buffer to a suitable composition for storage or use (e.g., by diafiltration or dialysis). An example of a commercial technology to form the initial LNP emulsion is the NanoAssemblr platform (e.g. NanoAssemblr Ignite, or NanoAssemblr Blaze) from Precision NanoSystems Inc.

The capped or uncapped RNA used for formulation into LNPs was produced according to the corresponding method described above in Examples 4 through 7.

When an LNP other than LMAX was used, a produced RNA was typically formulated into the LNP by complexing the RNA with: (i) a cationic lipid, sometimes called an ionizable lipid; ii) a cholesterol or cholesterol derivative; (iii) a structural lipid, sometimes called a phospholipid; and (iv) a PEG lipid.

The LNP-formulated RNA was produced at one of two different scales, which use the same microfluidic mixing chip but distinct production methods. When less than 60 μg of RNA was formulated into LNP in a single batch, Method A below was generally employed. When more than 60 μg of RNA was formulated into a single batch, Method B was generally employed.

### Method A (Generally for Production of <60 μg of LNP-RNA)

RNA was formulated into lipid nanoparticles (LNP) using microfluidic mixing via a commercial microfluidic chip (NanoAssemblr Ignite cartridge from Precision Nanosystems Inc.). Briefly, separate organic lipid mix and aqueous RNA solutions were simultaneously injected in parallel into the microfluidic mixing chip, which rapidly mixes the fluid streams to form a stable emulsion. In this Example, lipid mix or lipid mixture means a lipid solution.

Lipids were dissolved in ethanol to form a lipid mix with a total lipid concentration of typically 12.5-25 mM. An acidic aqueous mRNA solution (100 mM citrate, pH 3.0) was mixed with the lipid solution at a 3:1 volume:volume ratio, corresponding to a flow rate of 9 mL/min for aqueous mRNA and 3 mL/min for organic lipid mix for a total flow rate of 12 mL/min. An N/P ratio ranged from 5-20, calculated via cationic amines in the lipids and the anionic phosphate on mRNA, with a final lipid concentration in the resulting LNP of 2 mM.

After passing the entire volume through the microfluidic chip, the outflow LNP-RNAs were immediately diluted 50-fold with DPBS-CMF (e.g. 1 mL outflow added to 49 mL of DPBS-CMF), and then concentrated using an Amicon centrifugal filter unit (MilliporeSigma, Cat No. UFC910024). The final concentrations of the LNP-encapsulated RNA were measured as described above using the RiboGreen assay (described above), and typically fell between 10 ng/μL and 200 ng/μL.

### Method B (for Production of 60 μg or More of LNP-RNA)

RNA-LNP complexes at pilot-scale (greater than 4 mL of hydrated volume or more than 60 μg of RNA) were produced using dual remote syringe pumps (Legato; KdScientific, 78-8110DRS) connected in tandem to a NanoAssemblr Ignite cartridge (Precision Nanosystem). The lipid mixture and RNA were prepared as described above in Method A of this example.

After collection at the output of the microfluidic cassette, LNPs were dialyzed (D-Tube Dialyzer Mega, Novagen 71743-3) against FM01 for 6 h at 4° C. and then concentrated using an Amicon centrifugal filter unit (Millipore Sigma, UFC910024). Final encapsulated RNA concentrations were typically between 10-200 ng/μL and were measured using the above RiboGreen method.

### Example 10: Influenza Encrypted RNA can be Activated by Influenza A or Influenza B Infection

Influenza encrypted RNAs were complexed into LMAX-LNP formulations (per Example 8) and the ability of cells transfected with these encrypted RNAs to translate polypeptides of interest was measured in the presence or absence of influenza infection. Typically, the day before infection, cells were plated in individual wells of multiwell plates at the desired density. The density of cells was estimated empirically such that just before infection, the monolayer was approximately ~80% confluent. Shortly before infection (1 to 3 h), the cells from one well were counted to quantify the cell number and the virus inoculum calculated to achieve the desired MOI. At 0 hpi, wells were infected with the virus at the desired MOI. After infection, encrypted RNA was delivered to the infected cells by treatment with LMAX-LNP-encapsulated RNA. The level of a translated polypeptide of interest at various times after infection was determined as described above.

In this example, at 16 hours prior to infection, human 293T cells were plated in 96-well plates at a density of 5×10⁴ cells per well. At 0 hpi, these 293T cells were either mock-infected (MOI-0) or infected with one of strains of four strains of influenza A or influenza B: A/PR8 (H1N1), A/SwOH (H3N2), B/Yamagata, B/Victoria at an MOI of 3 as described above. At two hours post-infection, human 293T cells were treated with 6.25-200 ng of LMAX-LNP-formulated influenza A encrypted RNA (ERNA-IAV-002-GDura). At 24 hpi, the GDura containing supernatant was harvested and the amount of secreted luciferase measured by plate reader assay as described above.

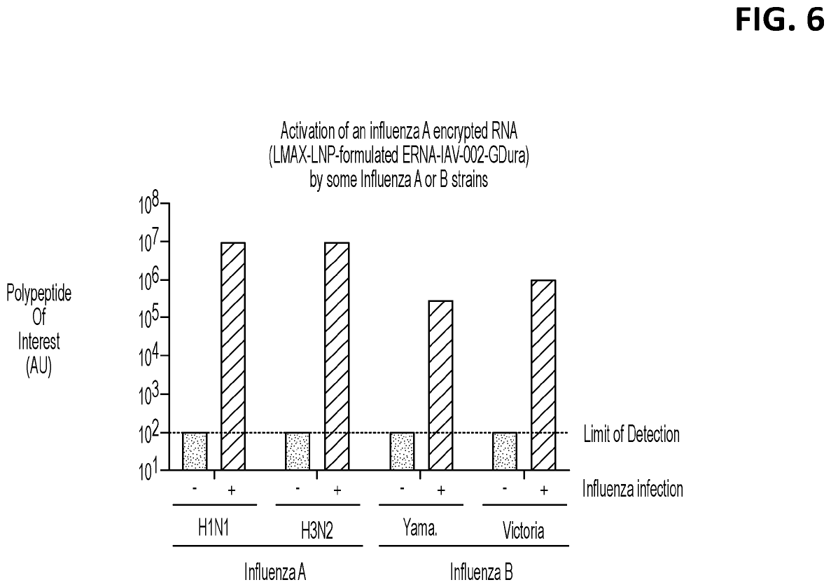

As shown in FIG. 6, cells treated with an LMAX-LNP-formulated influenza A encrypted RNA (ERNA-IAV-002-GDura) translated approximately background levels of the polypeptide of interest in the absence of influenza infection (as expected). In contrast, cells treated with the LMAX-LNP-formulated encrypted RNA translated high levels of the polypeptide of interest (GDura) after infection by a variety of influenza A or influenza B strains, including strains representing diverse influenza A subtypes (H1N1 or H3N2) or representative influenza B lineages (e.g., Yamagata or Victoria lineages). Levels of the polypeptide of interest increased by approximately 1,000-100,000× (i.e., 3-5 log) in influenza A or B infected cells treated with the encrypted RNA relative to the level of the polypeptide of interest in treated with the encrypted RNA but not infected with an influenza variant.

These results are consistent with previously reported findings that influenza minigenomes comprised of portions of influenza A or influenza B can be replicated by influenza A or influenza B RdRPs (Baker et al., J Viro (2014); DOI: 10.1128/JVI/01440-14).

The findings of this Example demonstrate that an influenza A encrypted RNA can be activated by a variety of influenza A or B strains. The findings of Example 17 below demonstrate that an influenza B encrypted RNA can be activated by a variety of influenza A or B strains.

Thus, the corresponding therapeutic influenza encrypted RNAs (e.g. with an interferon as the polypeptide of interest) can be activated by influenza A or influenza B strains. As a result, therapeutic encrypted RNA activation can extend beyond the level of species to target a viral genus (influenza A or B), even when species within the genus are genetically and antigenically distinct. Further, one or more therapeutic encrypted RNA sequences may be activated by the same translation activator and, inversely, distinct translation activators may activate the same therapeutic encrypted RNA.

Importantly, providing an encrypted RNA alone, either directly as an RNA or encoded as a DNA, and without complexation with influenza proteins (such as NP or M), can be sufficient to permit specific translation activation by a target-specific translation activator. Despite influenza viruses having genome replication occur primarily in the nucleus, providing encrypted RNA to the cell cytoplasm (e.g., via LNP-based transfection) can be sufficient to activate translation in the presence of the targeted translation activator. As RNA viruses (and all viruses) utilize the host to translate their mRNA (as they lack a complete set of translational components), viral proteins must migrate from the site of synthesis (a cytoplasmic ribosome) to other cellular destinations, which can be cytoplasmic or non-cytoplasmic. Consequently, there is an opportunity for encrypted RNA to contact viral polypeptides within the cytoplasm, even if viral replication occurs outside the cytoplasm.

### Example 11: Construction of Viral Vectors Harboring DNA-Encoded Encrypted RNA Cassettes

Lentiviral transfer vectors were constructed based upon standard 2nd generation lentiviral vector systems (Johnson et al., Mol. Ther. Meth. Dev. 2021, doi: 10.1016/j.omtm.2021.03.018) using the methods described in Example 1. Transfer vectors were developed that incorporated 5′ and 3′ flanking elements that flanked a DNA-encoded encrypted RNA cassette.

Additional sets of lentiviral transfer vectors were initially developed: pLVG03, pLVG04, pLVG05, pLVG06, and pLVG07. For their construction, pLVG03-based transfer vectors were comprised of a DNA-encoded encrypted RNA cassette flanked by a 5′ sequence (LV_5p_G03, SEQ ID NO: 358) and 3′ sequence (LV_3p_G03, SEQ ID NO: 359). The additional three families were constructed similarly: pLVG04 based vectors were comprised of a DNA-encoded encrypted RNA cassette flanked by a 5′ sequence (LV_5p_G04, SEQ ID NO: 360) and 3′ sequence (LV_3p_G04, SEQ ID NO: 361); pLVG05 based vectors were comprised of a DNA-encoded encrypted RNA cassette flanked by a 5′ sequence (LV_5p_G05, SEQ ID NO: 362) and 3′ sequence (LV_3p_G05, SEQ ID NO: 363); and pLVG06 based vectors were comprised of a DNA-encoded encrypted RNA cassette flanked by a 5′ sequence (LV_5p_G06, SEQ ID NO: 364) and 3′ sequence (LV_3p_G06, SEQ ID NO: 365). pLVG07 based vectors were developed for sarbecovirus encrypted RNAs (see Example 20 below) and were comprised of a DNA-encoded encrypted RNA cassette flanked by a 5′ sequence (LV_5p_G07, SEQ ID NO: 366) and a 3′ sequence (LV_3p_G07), SEQ ID NO: 367).

The form pLVG03-AT101-ERNA-IAV-001-GDura is used to indicate that an influenza A encrypted RNA encoding GDura utilizing the IAV-001 scaffold was cloned into the AT101 DNA cassette within the pLVG03 lentiviral transfer vector.

### Example 12: Production of Viral Vectors to Deliver DNA-Encoded Encrypted RNA Cassettes

### Lentiviral Vector Crude Production

A Lenti X 293T cell line (Clontech Labs, cat. No. 3P 632180) was typically used for lentiviral vector production. Cells were plated in sterile 15 cm polystyrene plates in D10 one day before transfection to attain 90-95% confluency the next day.

A set of three 2nd generation lentiviral packaging plasmids were used for cell transfection and lentivirus rescue:
psPax2 packaging plasmid;
VSV-G envelope expressing plasmid;
Lentiviral transfer vector encoding encrypted RNA described in Example 11.

In total, 30 μg of DNA was used for transfection of one 15-cm plate at molar ratio 7:1:6 of psPax2, VSV-G, and transfer vector plasmid, respectively.

323 mg of transfection reagent polyethyleneimine (PEI max, linear, molecular weight 40,000 (Polysciences, cat. No. 24765-1)) was dissolved in total 1 L of cell culture water, adjusted to pH 2-3, filter-sterilized, and aliquoted for storage at −20° C. The calculation of PEI, DPBS and DNA amounts was based on an optimal nitrogen: phosphate ratio of 27.5 in PEI and DNA, respectively (transfection calculator from Challis et al., Nature Protocols 2019). Precisely, ratio of 11:1 of PEI solution (μL): total DNA (μg) was used in total of 2 mL DPBS volume per one 15-cm plate.

At the time of transfection, PEI+DPBS master mix and the DNA+DPBS solution were prepared for each viral prep. Required volume of PEI+DPBS master mix was added dropwise to the DNA+DPBS solution while gently vortexing to mix. The tube was then thoroughly vortexed for 10 seconds to mix and incubated at ambient temperature (20-25° C.) for 10 minutes. Two mL of the transfection solution was added dropwise to each 15-cm plated with 20 mL media and swirled to mix before returning the dishes to the cell culture incubator. Transfection media was removed 12-24 h post transfection and replaced it with 15 mL of fresh D10. Media containing lentivirus was harvested 48 h after first media change and replaced by 15 mL of fresh D10. Finally, lentivirus was again harvested another 48 h later after second media change, two fractions of total volume 28-30 mL from a single 15-cm plate were combined.

### Purification

Collected solution was filtered through 0.45 μm PES membrane filter unit and 25 mL of filtered virus was added to a SW-28 ultracentrifuge tube. Filtrate was carefully underlaid with 5 mL of 6% (m/v) iodixanol solution. Final volume was carefully brought to 35 mL by adding an additional 5 mL of filtrate to each tube. Tubes were loaded in an “SW 28”-type swinging bucket rotor and spun at 2×10⁴ rpm for 90 min at 4° C. within an ultracentrifuge. Post-spin, supernatant was aspirated clinging to the rim and sides of tube. The viral vector pellet (not visible) was washed out and resuspended by pipetting in 300-1000 μL of D10 to make 10-100× fold concentrated lentivirus. Final viral preparation was aliquoted as appropriate, e.g., into 50-200 μL aliquots, and stored at −80° C. avoiding thaw-freeze cycles.

### Viral Titration

Lentivirus physical titers were determined using RT-qPCR Lentivirus Titer Kit (Applied Biological Materials, cat. No. LV900). For purified high titer viral samples (100× concentrated), virus was diluted to 0.1× or 0.01×. Low viral titer samples were used undiluted.

Briefly, 2 μL of the lentivirus preparation were mixed with 18 μL of virus lysis buffer and incubated at room temperature (20-25° C.) for 3 min. Five 10-fold serial dilutions of the standard control DNA were performed to plot calibration curve. RT-qPCR reactions were combined with viral samples and standard dilutions as follows:

TABLE 14

| Component | Volume |
|---|---|
| 2x qPCR MM | 10 μL |
| Primer mix | 2 μL |
| Sample, NTC, Standard | 2 μL |
| Nuclease-free water | 6 μL |

TABLE 15
qPCR cycling conditions

| Step | Temperature | Duration | Cycle |
|---|---|---|---|
| Reverse transcription | 42° C. | 20 min | 1 |
| Enzyme activation | 95° C. | 10 min | 1 |
| Denaturation | 95° C. | 15 sec | 30 |
| Annealing/Extension | 60° C. | 1 min | |

Lentivirus titer, Viral Genomes per mL (VG/mL) was determined using standard curve logarithmic regression of Cq value vs. virus titer for standard dilutions with known concentration, taking into consideration sample dilution factor.

### Example 13: An Influenza Encrypted RNA can be Activated by Target-Specific Translation Activators and is not Activated by RdRPs from Non-Influenza Viruses

Experiments were designed to test the specificity of induction of encrypted RNA activation by IAV as compared to off-target activation by unrelated viruses (RSV and EMCV). Lentiviral vectors (LV) encoding the DNA-encoded encrypted RNA cassette from pAT002-ERNA-IAV-002-GDura were used to transduce human cells. In particular, a preparation of a lentiviral vector using pLVG04-AT002-ERNA-IAV-002-GDura was produced (VSV-G pseudotyped). For simplicity, in this Example, LVG04-ERNA-GDura means lentiviral vectors prepared with pLVG04-AT002-ERNA-IAV-002-GDura.

In this assay, cells were transduced with LVG04-ERNA-GDura lentiviral vector carrying influenza A encrypted RNA and infected as previously with IAV, or with non-influenza viruses (e.g. RSV, OC43-CoV or EMCV).

At approximately 8 h prior to transduction with lentiviral vectors encoding DNA-encoded encrypted RNA cassettes, human 293T cells (1×10⁵ cells per well) or human A549-STAT^{-/-} cells (5×10⁴ cells per well) were plated in 12-well multiwell plates at the indicated densities. At the time of transduction, approximately 8 h after plating, cells were transduced with lentiviral vectors in the presence of 10 μg/mL polybrene and allowed to recover for 72 h. After the recovery period (72 h), cultures were replated in 48-well multiwell plates at a density of 5×10⁴ cells/well and allowed to recover overnight prior to virus infection.

At 0 hpi, cells were either mock-infected (MOI=0) or infected with one of four viruses at an MOI of 10: influenza A (H1N1), RSV A2, OC43, or EMCV. At 24 hpi, the GDura containing supernatant was harvested and the amount of secreted luciferase measured by plate reader assay as described above.

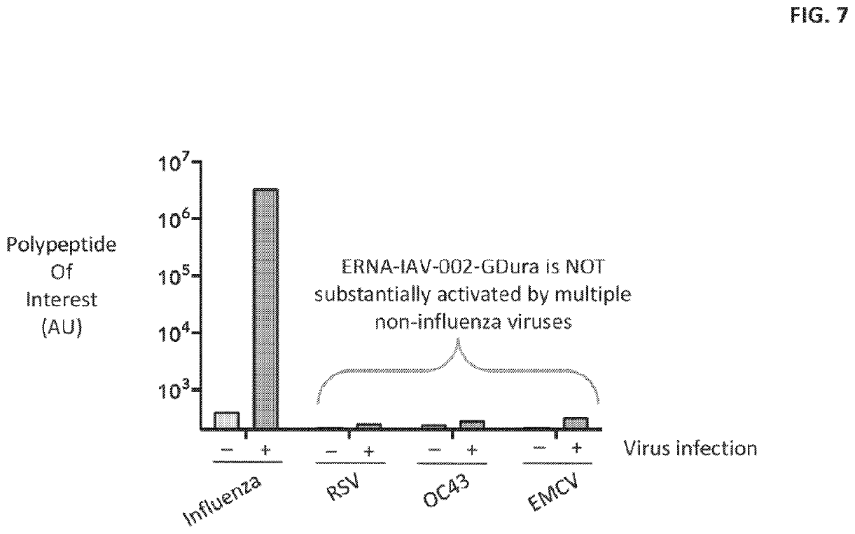

FIG. 7 shows that influenza infection of cells transduced with LVG04-ERNA-GDura resulted in activation of the influenza encrypted RNA. Infections of the cells with a non-influenza RNA virus did not substantially activate the influenza encrypted RNA. In particular, infections of cells transduced with LVG04-ERNA-GDura by some pneumoviruses (e.g., RSV), coronaviruses (e.g., OC43-CoV), or picornaviruses (e.g., EMCV) did not substantially activate the influenza encrypted RNA. None of these ‘off-target’ viruses encodes the influenza RdRP domains that enable substantial activation of the influenza encrypted RNA.

### Example 14: Encrypted RNAs can be Modified to Remove Key PAMPs while Retaining the Ability to be Efficiently Activated

Influenza A encrypted RNAs based on the ERNA-IAV-002-GDura scaffold were prepared via in vitro transcription from a template plasmid using the conditions specified above. RNA S158 was prepared using template plasmid pAT201-ERNA-IAV-002-GDura and an equimolar solution of ATP, CTP, GTP, and UTP to form the nucleotide pool. The resulting encrypted RNA (S158) is 5′-triphosphorylated. RNA S159 was prepared via IVT from template plasmid pAT221-ERNA-IAV-002-GDura, and the larger 5′-monophosphorylated encrypted RNA was purified away from the leading hammerhead ribozyme. RNA S160 was prepared analogously to S158 except the total “UTP pool” was comprised of a binary mixture of 30% UTP and 70% pseudouridine triphosphate (percentages are % by mol).

293T cells were treated with S158, S159, or S160 encrypted RNA that had been formulated into LNPs, and then the cells were infected with influenza A/PR8 at 16 hpi. The amount of luciferase signal was quantified in the supernatant, as described above.

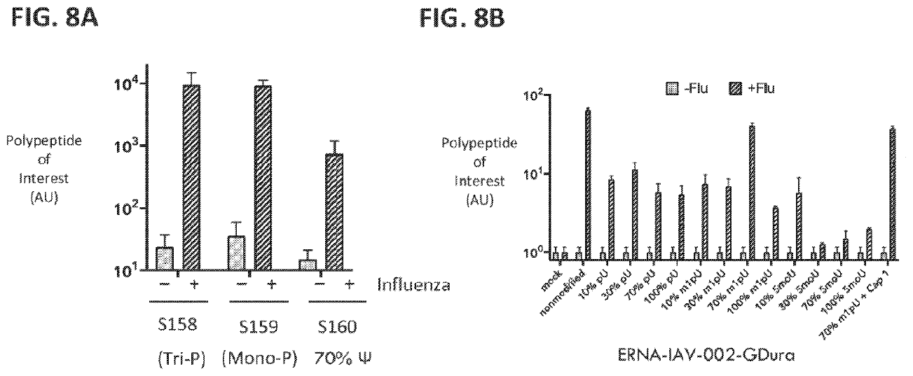

FIG. 8A shows that the tested encrypted RNAs were able to be activated by influenza infection and produced about a 1.5-3.0 log increase in luciferase protein levels after influenza infection, whether or not the encrypted RNAs retained all molecular features of influenza viral RNAs. For example, a 5′-triphosphorylated influenza encrypted RNA (S158), a 5′-monophosphorylated influenza encrypted RNA (S159), or a 5′-triphosphorylated influenza encrypted RNA incorporating 70% pseudouridine (S160) can each be substantially activated by influenza infection to produce the encoded polypeptide of interest.

Additional modifications of influenza encrypted RNAs were also prepared analogously to S158. For example, 4 additional encrypted RNAs (S161 through S164) were prepared analogously to S158, except that the UTP pool in the encrypted RNAs was comprised of one of the following binary mixtures: 10% N1-methylpseudouridine triphosphate and 90% UTP (in S161); 30% N1-methylpseudouridine triphosphate and 70% UTP (in S162); 70% N1-methylpseudouridine triphosphate and 30% UTP (in S163); or 100% N1-methylpseudouridine triphosphate and 0% UTP (in S164). The percentages are % by mol.

An additional 4 encrypted RNAs (S165 through S168) were prepared analogously to S158, except that the UTP pool in the encrypted RNAs was comprised of one of the following binary mixtures: 10% 5-methoxyuridine triphosphate and 90% UTP (in S165); 30% 5-methoxyuridine triphosphate and 70% UTP (in S166); 70% 5-methoxyuridine triphosphate and 30% UTP (in S167); or 100% 5-methoxyuridine triphosphate and 0% UTP (in S168). The percentages are % by mol.

An additional 4 encrypted RNAs (S169 through S172) were prepared analogously to S158, except that the UTP pool in the encrypted RNAs was comprised of one of the following binary mixtures: 10% pseudouridine triphosphate and 90% UTP (in S169); 30% pseudouridine triphosphate and 70% UTP (in S170); 70% pseudouridine triphosphate and 30% UTP (in S171); or 100% pseudouridine triphosphate and 0% UTP (in S172). The percentages are % by mol.

Each of these nucleotide modified encrypted RNAs (S161 through S172) can be substantially activated by a translation activator derived from influenza. Each influenza encrypted RNA was LNP-formulated and transfected into a population of human 293T cells. The 293T cells were co-transfected with a pool of plasmids encoding the PA, PB2, PB1, and NP proteins derived from Influenza A/PR8 following the approach in FIG. 8A. The amount of the translated polypeptide of interest (secreted GDura luciferase) from the encrypted RNAs was quantified at 24 hours post-transfection, as described above.

Other modifications of influenza encrypted RNAs were prepared analogously to S158 to test whether an encrypted RNA could tolerate modifications of the 5′ terminus. For example, influenza encrypted RNAs were prepared analogously to S158 but modified to incorporate a 5′-Cap structure (a Cap 1 structure generated via co-transcriptional capping by using CleanCap Reagent AG, TriLink BioTechnologies). An additional influenza encrypted RNA was prepared analogously to S158 but with no 5′ triphosphate due to additional phosphatase treatment with Calf Intestinal Alkaline Phosphatase, (CIP), New England Biolabs.

FIG. 8B shows that each of these modified encrypted RNAs was substantially activated during co-transfection of 293T cells with a pool of plasmids encoding the PA, PB2, PB1, and NP proteins derived from Influenza A/PR8, following the approach above.

In some embodiments, in addition to being activated by a translation activator derived from influenza, a 5′-Cap structure or modified nucleosides substantially reduced the immunogenicity of an encrypted RNA in A549 Dual cells, as quantified by the immunogenicity (ISRE) assay described below in Example 19. For example, an encrypted RNA where 70% of the UTP pool was modified to N1-methylpseudouridine triphosphate and the encrypted RNA was capped with the CleanCap AG 5′-Cap structure had immunogenicity (ISRE) levels ~10-fold lower than S158, and nearly equivalent levels of activation in the presence of the influenza-derived translation activator.

Thus, genomic regions that are known to be immunogenic PAMPs (e.g. uncapped 5′ triphosphates or polyuridine stretches) can be removed on some encrypted RNAs or modified on some encrypted RNAs—and the resulting encrypted RNAs can still retain the capability to be contacted and activated by a virus-derived translation activator. In some embodiments, encrypted RNAs can therefore be designed to encode immunogenic proteins, but to only trigger a substantial immune response upon contact with a translation activator (e.g. during viral infection).

### Example 15: Long-Term Persistence and Activation of DNA-Encoded Encrypted RNA Cassettes Delivered Via Viral Vector

Lentiviral vectors (LV) encoding the DNA-encoded encrypted RNA cassette from pAT002-ERNA-IAV-002-GDura were used to transduce human A549 cells at a multiplicity of infection (MOI) of approximately 1-2 as described above. In particular, separate preparations of lentiviral vectors using pLVG04-AT002-ERNA-IAV-002-GDura or pLVG04-AT002-ERNA-IAV-002-hu_IFNB were produced. For simplicity, in this Example, LVG04-ERNA-GDura means lentiviral vectors prepared with pLVG04-AT002-ERNA-IAV-002-GDura and likewise LVG04-ERNA-hu_IFNB means lentiviral vectors prepared with pLVG04-AT002-ERNA-IAV-002-hu_IFNB.

### Lentivirus Transduction

Low passage AD293 cells and A549 cells were each plated in 8 wells of 48-well plates, 1.3×10⁵ cells/well in 150 μL of D10 media at one day prior to transduction. On the following day (typically 16-24 h later), three wells of each cell line were transduced with LVG04-ERNA-GDura, three wells were transduced with LVG04-ERNA-hu_IFNB, while two wells remained controls. Transduction was performed with lentiviral vectors at an MOI of 10. To achieve this, 7.5 μL of lentiviral viral prep with titer 2.1×10⁸ vg/mL was diluted in 50 μL of media containing 5 μg/mL polybrene (MilliporeSigma, cat. No. TR-1003) to enhance transduction, and added directly to cells in 150 μL media. Control wells received 50 μL of D10 only. After adding lentiviral vector to cells, the cultures were briefly mixed, then allowed to recover in humidified 5% CO₂ incubators at 37° C.

At 3 days post-transduction (dpt) one control well per cell line and each of lentiviral vector transduced wells (AD293 and A549 transduced with LVG04-ERNA-GDura or LVG04-ERNA-hu_IFNB) were removed and analyzed by flow cytometry (Beckman-Coulter, CytoFlex S flow cytometer) to monitor transduction efficiency through RFP fluorescent tag expression.

Out of remaining transduced replicates, one full transduction set along with associated controls was split and replated to maintain the lineages in continuous culture through the time-course experiment.

### PR8 Influenza Infection

The remaining set of AD293 and A549 (transduced with LVG04-ERNA-GDura or LVG04-ERNA-hu_IFNB) were infected with influenza A/PR8 at various timepoints, beginning at 3 days post-transduction. First, 150 μL media from each well plus control media were collected into tubes for further analysis. Remaining media was replaced with 50 μL of infection media (DPBS-CMF supplemented with 0.04% (m/v) BSA and filter-sterilized), containing PR8 at an MOI of 5 (superinfection) without adding TPCK-trypsin. Influenza inocula were incubated with cells for 2 h at 37° C. with frequent swirling of the infection media across the monolayer surface. After 2 h of incubation, 150 μL of FreeStyle 293 Expression Medium (ThermoFisher, cat. No. R79007) was added directly to the infection media. PR8-infected cells were incubated for 48 h and 150 μL of media was collected at both 24 h and 48 h. Media samples before and after influenza infection were stored frozen at −20° C. until performing quantifying the amount of secreted luciferase. The set of cells maintained further in culture was split and replated as needed and used for A/PR8 influenza infections at the following time-points through the time-course experiment. Samples were obtained at day 3 post-transduction, and subsequently at 7 day intervals post-transduction from day 7 to day 35 (day 7, day 14, . . . , day 35), and at 14 day intervals thereafter (day 49, day 63, day 77, . . . ).

### Quantification of Translated GDura or Human IFN-β (Hu_IFNB)

Media samples, collected from LVG04-ERNA-GDura or LVG04-ERNA-hu_IFNB transduced cells (before and after PR8 infection) were analyzed via luminescence assay by adding 50 μL QUANTI-Luc luciferase substrate (InvivoGen, cat. No. rep-qlc2) to 20 μL sample in duplicates and measuring luminescence signal immediately on plate reader (PerkinElmer).

Media samples, collected from LVG04-ERNA-GDura or LVG04-ERNA-hu_IFNB transduced cells (before and after PR8 infection) were analyzed using human IFN-β bioluminescent ELISA kit (InvivoGen, cat. No. luex-hifnbv2). Briefly, 96-well plates were pre-coated with human IFN-β capture antibody, the mixture of 50 μL sample or standard sample dilutions and 50 μL Lucia-conjugated detection antibody (30 ng/mL) was added to the plate, sealed, and incubated for 2 hours at 37° C. After incubation, plates were thoroughly washed 3 times with PBST three times, then 50 μL of QUANTI-Luc luciferase substrate was added and luminescence signal detected immediately on plate reader (PerkinElmer). The human IFN-β concentration in samples was determined using standard curve logarithmic regression of fluorescence intensity value vs. concentration of standard sample dilutions with known concentration, taking into consideration any sample dilution factor used.

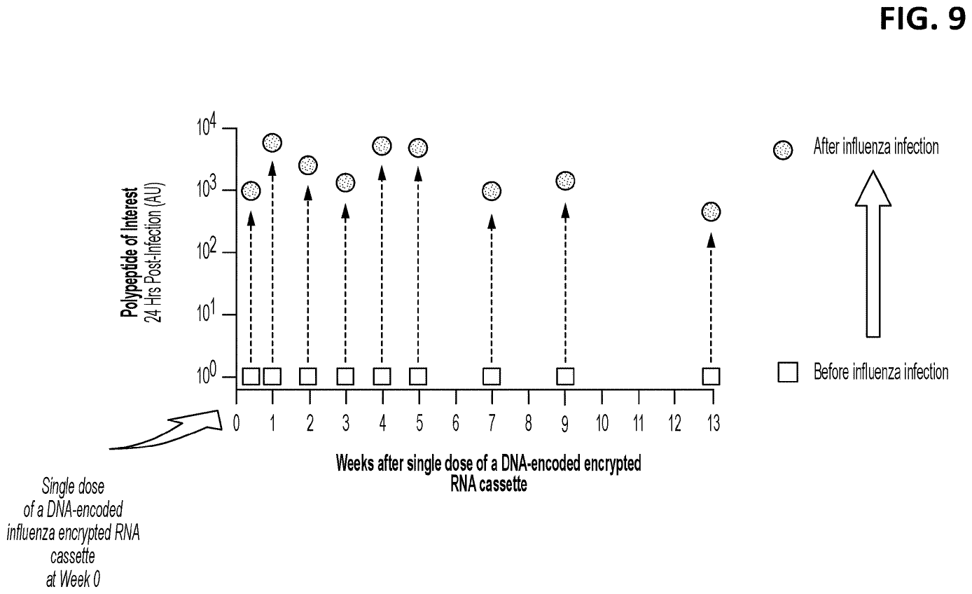

FIG. 9 shows that an influenza encrypted RNA can be activated up to at least 13 weeks later after delivery into cells by a DNA-encoded influenza encrypted RNA cassette. As described in detail above, AD293 cells were transduced with LVG04-ERNA-GDura on Day 0, and production (and secretion) of the polypeptide of interest (GDura) was repeatedly quantified during a 13-week study in the presence or absence of influenza infection (A/PR8). Low levels of the polypeptide of interest were measured at each time point of the study in the absence of influenza infection. However, an approximately 103-104× increase in GDura protein production (in comparison to the level of GDura in the absence of virus infection) was quantified at each time point in the presence of influenza infection. Notably, the cells were only treated with the DNA-encoded encrypted RNA a single time during the study, on Day 0.

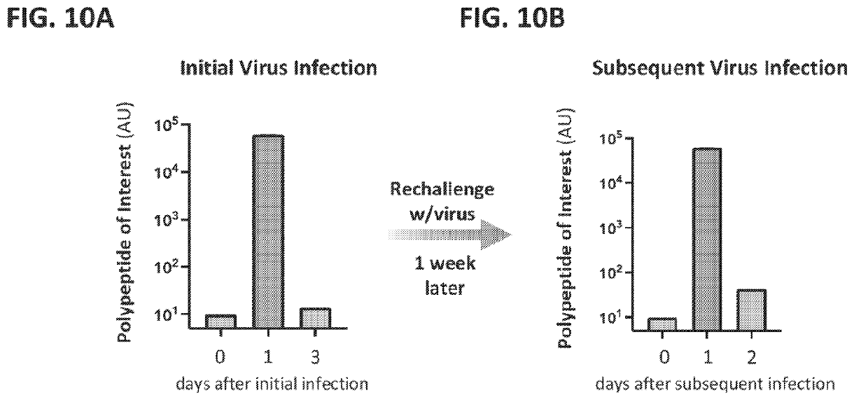

FIGS. 10A-10B show that, in some embodiments, an encrypted RNA can be activated multiple times after a single of treatment of cells with a DNA-encoded influenza encrypted RNA cassette. As in FIG. 9, AD293 cells were transduced with LVG04-ERNA-GDura once, and production of the polypeptide of interest (GDura) was then quantified in the presence or absence of repeat influenza infections (A/PR8). FIG. 10A shows that after the first influenza infection of the treated cells, GDura protein levels increased up to approximately 10,000× (4 log) within 1 day, before they declined to approximately baseline levels within 3 days, coincident with a reduction of viral infection. FIG. 10B shows that when influenza A/PR8 virus was re-introduced into the culture (1 week later)—without an additional encrypted RNA treatment—the encrypted RNA was again reactivated to produce a high level of GDura (~4 log above the level immediately prior to re-infection), within 1 day of re-infection. The reactivation of the encrypted RNA shows that its deactivation upon viral clearance is not solely an artifact of selection for cells that lack the encrypted RNA.

### Example 16: Durable Antiviral Activity of an Influenza Therapeutic Encrypted RNA In Vitro

To test if the persistence of a DNA-encoded encrypted RNA described in Example 16 could result in durable antiviral efficacy, cell populations analogous to those in Example 15 were treated with a DNA-encoded therapeutic encrypted RNA cassette (LVG04-ERNA-hu_IFNB) and tracked over a contemporaneous 13-week study.

Following the approach described in detail in Example 15, A549 cells were transduced with LVG04-ERNA-hu_IFNB once on Day 0, and production (and secretion) of the polypeptide of interest (human IFN-β) was repeatedly quantified during a 13-week study in the presence or absence of influenza infection (A/PR8). At each sampled timepoint during the study, the levels of human IFN-β increased by 10-100× or more upon influenza viral infection of the treated cells.

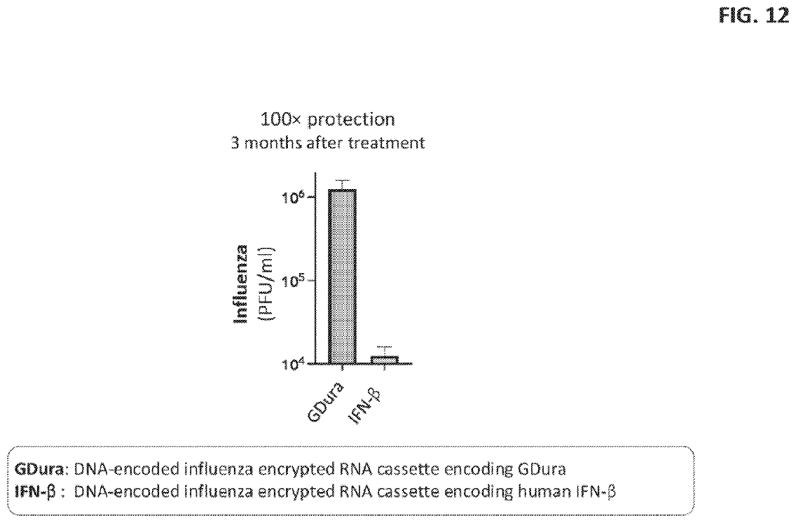

FIG. 12 shows the antiviral efficacy of LVG04-ERNA-hu_IFNB. At the end of the 13-week study, the A549 cells treated with LVG04-ERNA-hu_IFNB were compared with the A549 cells treated with LVG04-ERNA-GDura (described in Example 15). Each cell population was treated only a single time approximately 13 weeks prior to this point. Both sets of cells were then challenged with A/PR8 at MOI 0.01. The level of viral replication in A549 cells treated with the DNA-encoded therapeutic encrypted RNA (LVG04-ERNA-hu_IFNB) was approximately 2 log (100×) lower than the level of influenza in the parallel A549 cells treated with the control encrypted RNA (LVG04-ERNA-GDura), as measured by a FFU assay, as described above.

### Example 17: Influenza B Encrypted RNA can be Successfully Activated by Influenza A Infection or Influenza B Infection

Human 293T cells were transfected with 100 ng of an influenza B encrypted RNA expressing plasmid (pAT002-ERNA-IBV-001-GDura). Sixteen hours later, cells were either mock-infected (MOI-0) or infected with one of four strains of influenza A or influenza B: A/PR8 (H1N1), A/SwOH (H3N2), B/Brisbane (Victoria lineage), or A/NewCaledonia (H1N1) at an MOI ranging between 0 and 3, as described above. At 48 h after infection, the GDura containing supernatant was harvested and the amount of secreted luciferase measured by plate reader assay, as described above.

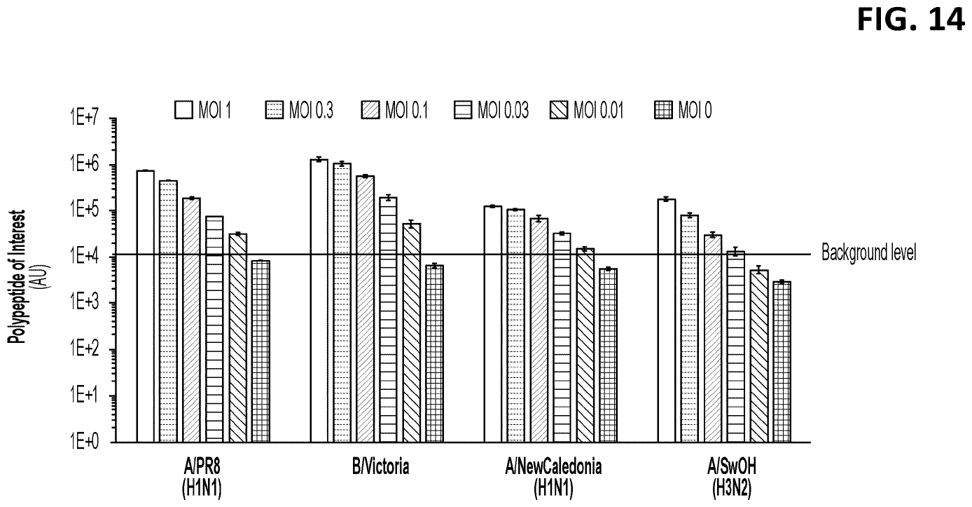

As shown in FIG. 14, all transfections yielded approximately background levels of translation of the polypeptide of interest (GDura) in the absence of influenza infection (MOI of 0). Further, the influenza B antisense encrypted RNA encoding GDura (produced from the pAT002-ERNA-IBV-001-GDura plasmid) was able to be activated in cells infected with influenza A (multiple H1N1 subtypes and H3N2 subtypes) or influenza B/Brisbane in an MOI-dependent manner.

These results mirror the observations in Example 10, demonstrating that influenza encrypted RNAs (including influenza therapeutic encrypted RNAs), whether inspired by influenza A or influenza B genomes, can be activated by influenza A or influenza B. Notably, the same influenza B strain (B/Brisbane) which efficiently activated the influenza B encrypted RNA in this Example, efficiently activated the influenza A encrypted RNA in Example 10 (wherein the influenza strain is labelled as B/Victoria, for its lineage). Thus, Examples 10 and 17 collectively show that, in some embodiments, distinct encrypted RNAs can be activated by the same translation activator, or, in some embodiments, the same encrypted RNA can be activated by distinct translation activators. Activation of encrypted RNAs can therefore extend beyond the level of species to target a viral genus (e.g. influenza A & B), even when the species within the genus are genetically and antigenically distinct.

### Example 18: Construction of Sarbecovirus Encrypted RNAs

### Sarbecovirus Sense Encrypted RNA Scaffolds

DNA sequences were cloned by standard molecular biology methods as described in Example 1. For sarbecovirus sense encrypted RNAs, some L and R flanking nucleotide sequences were obtained by concatenation of nucleotide regions from a publicly available SARS-COV-2 genome sequence (NCBI GenBank ID: NC_045512.2). All numbering is in reference to this nucleotide sequence. The 1501 nt L flanking sequence (sense_5p_SARS2, SEQ ID NO: 68) is comprised of a concatenation of three regions of nucleotides of SARS-COV-2 genome: a 789 nucleotide region (nt 1-789); a 667 nucleotide region (nt 19674-20340); and a 45 nucleotide region (28229-28273). The R flanking sequence (sense_3p_SARS2, SEQ ID NO: 130) is comprised of a 370 nt contiguous nucleotide region from nt 29534-29903.

Alternative SARS-COV-2 sense encrypted RNA sequences were also developed, which included additional sequences within the L flanking sequence not present in sense_5p_SARS2 (SEQ ID NO: 68) to further lower translation in the absence of a target-specific translation activator and increase translation in the presence of the translation activator. When compared to sense_5p_SARS2 (SEQ ID NO: 68), an alternative L flank sequence was constructed (sense_5p_SARS2_N250, SEQ ID NO: 109) that contained an additional 250 nt region of N (nt 28479-28726) and two stop codons (“N250”), positioned within the L flank. An analogous L flank sequence was also developed which introduced a stop codon into the retained portion of orf1ab (sense_5p_SARS2_N250_stop, SEQ ID NO: 113) to reduce the potential for extended translation of the orf1ab reading frame.

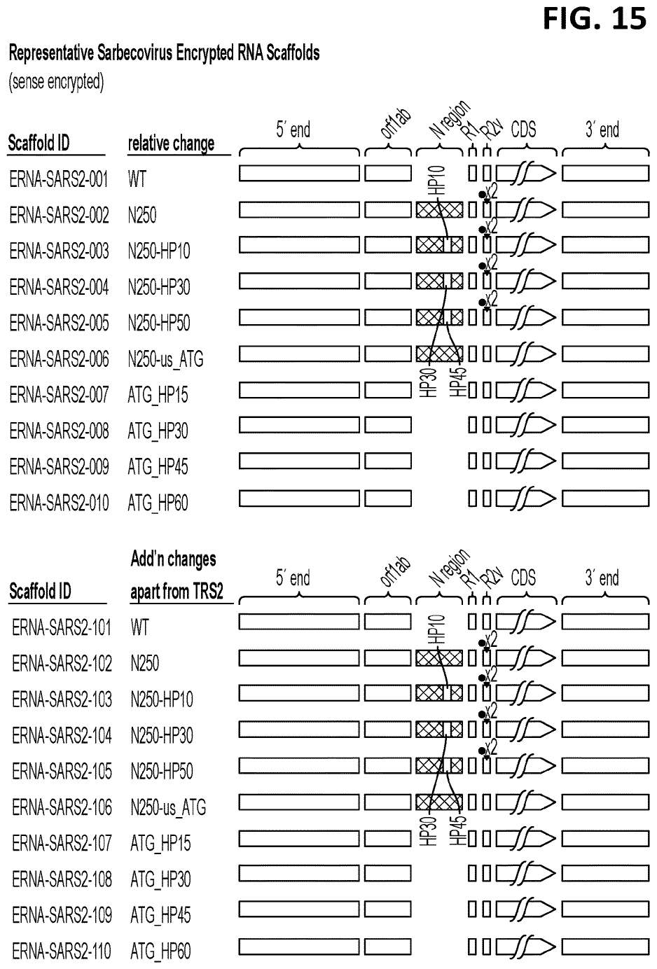

In a similar way, sense_5p_SARS2_N250_HP10 (SEQ ID NO: 110), contained the N250 sequence with inclusion of a hairpin structure situated within the N250 sequence, with a predicted free energy of folding of 10 kcal/mol (HP10). Predicted thermodynamic stabilities of hairpin structures were computed using the online UNAFold web service (unafold.org) RNA Folding Form V2.3 with default parameters to calculate the free energy of folding. Using this convention, sense_5p_SARS2_N250_HP30 (SEQ ID NO: 111) and sense_5p_SARS2_N250_HP50 (SEQ ID NO: 112) were constructed analogously, except the 10 kcal/mol hairpin in “N250_HP10” was replaced with progressively longer and more stable hairpins. N250_HP10 was substituted with a 30 kcal/mol hairpin in sense_5p_SARS2_N250_HP30 (SEQ ID NO: 111) or a 50 kcal/mol in sense_5p_SARS2_N250_HP50 (SEQ ID NO: 112). An L flanking sequence without a supplementary hairpin but with the addition of an upstream ATG sequence was also developed: sense_5p_SARS2_N250_us_ATG (SEQ ID NO: 114). These modified sequences are represented in a simplified schematic in FIG. 15.

Additional L flank sequences were further developed for use in sarbecovirus sense encrypted RNA scaffolds to reduce the potential for the encoded protein payload to be translated absent a translation activator (e.g. absent viral infection). With respect to sense_5p_SARS2 (SEQ ID NO: 68), sense_5p_SARS2_ATG_HP15 (SEQ ID NO: 104) contains a nucleotide sequence upstream of the last 45 nt of the L flank, designed to form an extended hairpin or stem-loop structure with the nucleotide sequence within the L flank preceding the ATG codon. The predicted free-energy of folding of the extended hairpin structure is 15 kcal/mol. Similarly, three additional L flank sequences were developed via a parallel approach: sense_5p_SARS2_ATG_HP30 (SEQ ID NO: 105), sense_5p_SARS2_ATG_HP45 (SEQ ID NO: 106), and sense_5p_SARS2_ATG_HP60 (SEQ ID NO: 107). In the “ATG_HP” family of sequences, the two numbers following HP in the name indicate the predicted stability of the hairpin structure in kcal/mol (e.g., “ATG_HP45” has a hairpin structure with predicted Gibbs Free Energy of folding of 45 kcal/mol). These modified sequences are represented in a simplified schematic in FIG. 15.

Alternative strategies to reduce the potential for the encoded protein payload to be translated absent a translation activator were also developed. For example, three L flank sequences-sense_5p_SARS2_hp_ATG2 (SEQ ID NO: 119), sense_5p_SARS2_hp_ATG3 (SEQ ID NO: 119), and sense_5p_SARS2_uATG_overlap (SEQ ID NO: 127)—were engineered to reduce background translation by masking the payload start codon within the secondary structure or by introducing upstream “decoy” start codons that would precede the sequence encoding the polypeptide of interest. An additional approach yielded L flank sequences sense_5p_SARS2_uATG_NDegron1 (SEQ ID NO: 125) and sense_5p_SARS2_uATG_NDegron2 (SEQ ID NO: 126), which added “N Degron” sequences to the subsequent payload CDS to accelerate degradation of the polypeptide of interest if the polypeptide of interest is translated absent a translation activator.

Additional L flank sequences-sense_5p_SARS2_UTRuATGv1 (SEQ ID NO: 116), sense_5p_SARS2_UTRuATGv2 (SEQ ID NO: 117) or sense_5p_SARS2_UTRuATGv3 (SEQ ID NO: 118)—were engineered to further reduce background translation.

Additional L flank sequences were developed that harbored amino acid substitutions within the portion of non-structural protein 1 (nsp1) present in some sarbecovirus encrypted RNA scaffolds. The mutation set “nsp1_124” comprises two point mutations: mutating amino acid 124 from arginine to alanine (R124A) and amino acid 125 from lysine to arginine (K125A). The mutation set “nsp1_164” comprises two point mutations: mutating amino acid 164 from lysine to alanine (K164A) and amino acid 165 from histidine to alanine (H165A). Mutation set “nsp1_124_164” combined all four substitutions of “nsp1_124” and “nsp1_164”: R124A, K125A, K164A, and H165A. As an example, sense_5p_SARS2 was used a basis to generate sense_5p_SARS2_nsp1_124 (SEQ ID NO: 122), sense_5p_SARS2_nsp1_164 (SEQ ID NO: 124) and sense_5p_SARS2_nsp1_124_164 (SEQ ID NO: 123).

A 5′-extended L sequence, sense_5p_SARS2_GGC_HHRz (SEQ ID NO: 108) was developed to add compatibility with the co-transcriptional CG RNA dinucleotide (GpG RNA Dinucleotide [5′-3′], Trilink; Cat. No. O-31011) and subsequent hammerhead ribozyme sequence to the L flank.

An alternative L flank sequence, sense_5p_SARS2_PolII_ASA_ASD (SEQ ID NO: 115) was developed to reduce the potential for internal splicing within a sarbecovirus encrypted RNA (during transcription from a chromosomal or episomal vector within the nucleus) by removing classical splice acceptor and splice donor sequences.

For each of the L flank sequences described above (SEQ ID NOs: 68, 104-115, 119, and 122-127), (refer to Table 1 and Table 2) an additional corresponding L flank sequence was prepared to allow use of the sequence with a BSL-2 SARS-CoV-2 generation-limited (GL) infection model (SEQ ID NOs: 69-82 and 85-90). This was performed in order to test the activation of sarbecovirus sense encrypted RNAs containing the L flank sequence within a BSL-2 laboratory. In each of the modified sequences, the two TRS sequences (the first at the interval homologous to nt 70-75 of NC_045512 and the second at the interval homologous to nt 28260-28265 of NC_045512) were switched from ACGAAC to CCGGAT for use with a generation-limited (GL) viral system.

Certain GL compatible L flank sequences were labelled SEQ ID NOs: 69-82 and 85-90, specifically: (i) sense_5p_SARS2GL (SEQ ID NO: 69);
(ii) sense_5p_SARS2GL_N250 (SEQ ID NO: 75);
(iii) sense_5p_SARS2GL_N250_HP10 (SEQ ID NO: 76);
(iv) sense_5p_SARS2GL_N250_HP30 (SEQ ID NO: 77);
(v) sense_5p_SARS2GL_N250_HP50 (SEQ ID NO: 78);
(vi) sense_5p_SARS2GL_N250_us_ATG (SEQ ID NO: 80);
(vii) sense_5p_SARS2GL_ATG_HP15 (SEQ ID NO: 70);
(viii) sense_5p_SARS2GL_ATG_HP30 (SEQ ID NO: 71);
(ix) sense_5p_SARS2GL_ATG_HP45 (SEQ ID NO: 72);
(x) sense_5p_SARS2GL_ATG_HP60 (SEQ ID NO: 73);
(xi) sense_5p_SARS2GL_N250_stop (SEQ ID NO: 79);
(xii) sense_5p_SARS2GL_hp_ATG2 (SEQ ID NO: 82);
(xiii) sense_5p_SARS2GL_uATG_overlap (SEQ ID NO: 90);
(xiv) sense_5p_SARS2GL_uATG_NDegron1 (SEQ ID NO: 88);
(xv) sense_5p_SARS2GL_uATG_NDegron2 (SEQ ID NO: 89);
(xvi) sense_5p_SARS2GL_nsp1_124 (SEQ ID NO: 85);
(xvii) sense_5p_SARS2GL_nsp1_164 (SEQ ID NO: 87);
(xviii) sense_5p_SARS2GL_nsp1_124_164 (SEQ ID NO: 86);
(xix) sense_5p_SARS2GL_GGC_HHRz (SEQ ID NO: 74); and
(xx) sense_5p_SARS2GL_PolII_ASA_ASD (SEQ ID NO: 81).

Additional L and R flank sequences were generated that used a SARS-COV-1 genomic sequence as a basis. For sarbecovirus sense encrypted RNAs based on SARS-COV-1, an L flanking nucleotide sequence and an R flanking nucleotide sequence were obtained by concatenation of nucleotide regions from a publicly available SARS-COV-1 Urbani genome sequence (NCBI GenBank ID: AY278741.1). The L flanking sequence developed was labelled sense_5p_SARS1 (SEQ ID NO: 60) and the R flanking sequence was labelled sense_3p_SARS1 (SEQ ID NO: 129).

Similarly, sense_5p_SARS1 (SEQ ID NO: 60) was used as a basis to generate three additional sequences: sense_5p_SARS1_nsp1_124 (SEQ ID NO: 65), sense_5p_SARS1_nsp1_164 (SEQ ID NO: 67), and sense_5p_SARS1_nsp1_124_164 (SEQ ID NO: 66). Four analogous GL virus-compatible sequences with modified TRS sequences were also developed for the “sense_5p_SARS1-series” of L flank sequences: sense_5p_SARS1 (SEQ ID NO: 61), sense_5p_SARS1_nsp1_124 (SEQ ID NO: 62), sense_5p_SARS1_nsp1_164 (SEQ ID NO: 64), and sense_5p_SARS1_nsp1_124_164 (SEQ ID NO: 63).

Control L flank sequences, sense_5p_SARS2_nonactivatable (SEQ ID NO: 121) and sense_5p_SARS2GL_nonactivatable (SEQ ID NO: 84), were also developed with the reduced capacity to be activated by target translation activators by deletion of one of the TRS sites from sense_5p_SARS2 (SEQ ID NO: 68) or sense_5p_SARS2GL (SEQ ID NO: 69), respectively.

The initial sarbecovirus sense encrypted RNAs scaffolds were obtained by flanking a coding sequence for the polypeptide of interest on the 5′ side with an L sequence described above and on the 3′ side with sense_3p_SARS2 (SEQ ID NO: 130). For example, when GDura was the polypeptide of interest, sarbecovirus sense encrypted RNAs were constructed on these scaffolds by flanking a GDura coding sequence (CDS_GDura, SEQ ID NO: 273) on the 5′ side with an L sequence described above and on the 3′ side with sense_3p_SARS2 (SEQ ID NO: 130). ERNA-SARS2-001 was generated by concatenation of sense_5p_SARS2 (SEQ ID NO: 68), a coding sequence for the polypeptide of interest, and sense_3p_SARS2 (SEQ ID NO: 130) in 5′ to 3′ order. Additional sarbecovirus sense encrypted RNA scaffolds (ERNA-SARS2-002 through ERNA-SARS2-020) were constructed by a similar process, except the L sequence was selected from SEQ ID NOs: 104-115, 119, and 122-127. Sequences were used in order, with ERNA-SARS2-002 using SEQ ID NO: 109 and ERNA-SARS-020 using SEQ ID NO: 115. This construction strategy is depicted, in part, in a simplified schematic in FIG. 15.

Additional sarbecovirus sense encrypted RNA scaffolds incorporating modified TRS sequences for use with a BSL-2 GL virus were constructed similarly (ERNA-SARS2-101 through ERNA-SARS2-120). In this series, each scaffold used a different L sequence selected from SEQ ID NOs: 69-82 and 85-90, while the R sequence (sense_3p_SARS2, SEQ ID NO: 130) remained constant. For example, when GDura was the polypeptide of interest, each scaffold used a different L sequence selected from SEQ ID NOs: 69-82 and 85-90, while the CDS (CDS_GDura, SEQ ID NO: 273) and R sequence (sense_3p_SARS2, SEQ ID NO: 130) remained constant. L sequences were selected in numerical order, with ERNA-SARS2-101 using SEQ ID NO: 69 and ERNA-SARS2-120 using SEQ ID NO: 81. This construction strategy is depicted, in part, in a simplified schematic in FIG. 15. Complete details about the scaffolds are in Table 2.

Sarbecovirus sense encrypted RNA scaffolds employing “SARS1” L- or R-flanking sequences were additionally developed as listed in Table 2. An L-flank sequence was selected from SEQ ID NOs: 60, 65, 67, 66, 61, 62, 64, or 63 while the R-flank sequence was always sense_3p_SARS1 (SEQ ID NO: 129).

To test additional L- and R-flanking sequences for encrypted RNA function, 4 constructs were developed with 270-300 nt deletions in the orf1ab part of the L-flanking region: sense_5p_SARS2GL_Δ300_locA, sense_5p_SARS2GL_Δ300_locB, sense_5p_SARS2GL_Δ300_locC, and sense_5p_SARS2GL_Δ300_locD (SEQ ID NO: 91-94). Among the constructs with 270-300 nt deletions, only the scaffolds with sense_5p_SARS2GL_Δ300_locA (SEQ ID NO: 91) or sense_5p_SARS2GL_Δ300_locB (SEQ ID NO: 92) had activity that was within 10× the level of the corresponding undeleted L flank sequence when used with the same R flank sequence.

In addition, 9 constructs with 30 nt deletions of the L-flanking sequence were created to test the functionality of varied regions throughout the L-flanking sequence: sense_5p_SARS2GL_Δ30_loc01, sense_5p_SARS2GL_Δ30_loc02, sense_5p_SARS2GL_Δ30_loc03, sense_5p_SARS2GL_Δ30_loc04, sense_5p_SARS2GL_Δ30_loc05, sense_5p_SARS2GL_Δ30_loc06, sense_5p_SARS2GL_Δ30_loc07, sense_5p_SARS2GL_Δ30_loc08, and sense_5p_SARS2GL_Δ30_loc09 (SEQ ID NO: 95-103). Similarly, 5 partially deleted versions of R-flanking sequence were also developed: sense_3p_SARS2GL_Δ30_loc10, sense_3p_SARS2GL_Δ30_loc11, sense_3p_SARS2GL_Δ30_loc12, sense_3p_SARS2GL_Δ30_loc13, and sense_3p_SARS2GL_Δ30_loc14 (SEQ ID NO: 131-135). Among the constructs with 30 nt deletions, only the scaffold with the L region denoted sense_5p_SARS2GL_Δ30_loc02 (SEQ ID NO: 96) had activity that was within 10× of ERNA-SARS2-101.

Other sarbecovirus encrypted RNA scaffolds not explicitly listed here are detailed in Table 2.

### Sarbecovirus Antisense Encrypted RNA Scaffolds

For construction of some sarbecovirus antisense encrypted RNAs, L and R flanking nucleotide sequences were obtained by concatenation of nucleotide regions from a publicly available SARS-COV-2 genome sequence (NCBI GenBank ID: NC_045512.2). All numbering is in reference to this nucleotide sequence. The first antisense L flank developed (antis_5p_SARS2, SEQ ID NO: 138) is a 352 nt subsequence of the reverse complement of sense_3p_SARS2 (SEQ ID NO: 130) where the poly(A) tract has 18 fewer A. The initial R flank sequence developed (antis_3p_SARS2, SEQ ID NO: 145) is the reverse complement of sense_5p_SARS2 (SEQ ID NO: 68).

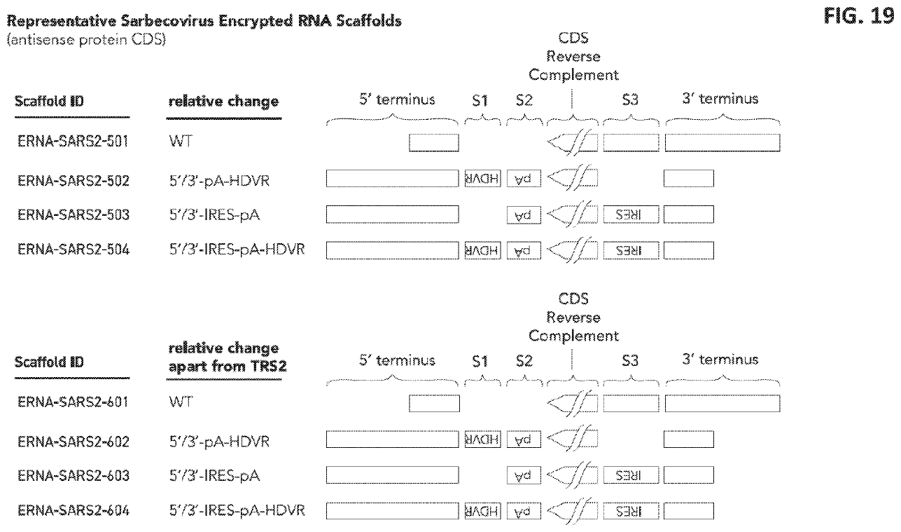

A collection of alternative L and R flanking sequences were also constructed to support the translation of the coding sequence from an IRES sequence when converted to a positive sense transcript during sarbecovirus replication. This strategy employed using L flanking sequences homologous to sense_5p_SARS2 (SEQ ID NO: 68) and R flanking sequences homologous to sense_3p_SARS2 (SEQ ID NO: 130). Two alternative L flank sequences were initially developed, incorporating: (i) an inverted poly(A) tract (antis_5p_SARS2_invert_pA, SEQ ID NO: 143); or (ii) both an inverted poly(A) tract and an adjacent inverted antigenomic hepatitis delta virus ribozyme (antis_5p_SARS2_invert_pA_HDVR, SEQ ID NO: 144). The corresponding novel R flank sequence (antis_3p_SARS2_invert_IRES, SEQ ID NO: 147) introduced an inverted IRES sequence. Some details of these constructs are explained graphically in a simplified schematic in FIG. 19.

To ensure compatibility with the BSL-2 generation-limited viral infection system, a parallel set of L or R flanking sequences was created for each sequence that modified critical TRS sequences situated just before orf1ab. For each of the L or R flank sequences to be changed, the TRS sequences were switched from ACGAAC to CCGGAT as described above. The following three new GL-compatible sequences were generated: antis_5p_SARS2_invert_pA (SEQ ID NO: 143) was converted to antis_5p_SARS2GL_invert_pA (SEQ ID NO: 140); antis_5p_SARS2_invert_pA_HDVR (SEQ ID NO: 144) was converted to antis_5p_SARS2GL_invert_pA_HDVR (SEQ ID NO: 141); and antis_3p_SARS2 (SEQ ID NO: 145) was converted to antis_3p_SARS2GL (SEQ ID NO: 146). A simplified schematic of these constructions is presented in FIG. 19.

The initial four sarbecovirus antisense encrypted RNA scaffolds were obtained by flanking the reverse complement of the coding sequence of a polypeptide of interest on the 5′ side with an L sequence described above and on the 3′ side with either antis_3p_SARS2 (SEQ ID NO: 145) or antis_3p_SARS2_invert_IRES (SEQ ID NO: 146). For example, ERNA-SARS2-501-GDura was generated by concatenation of antis_5p_SARS2 (SEQ ID NO: 138), rcCDS_GDura (SEQ ID NO: 301), and antis_3p_SARS2 (SEQ ID NO: 145) in 5′ to 3′ order. Similarly, ERNA-SARS2-502-GDura was generated by concatenation of antis_5p_SARS2_invert_pA_HDVR (SEQ ID NO: 144), rcCDS_GDura (SEQ ID NO: 301), and sense_3p_SARS2 (SEQ ID NO: 130) in 5′ to 3′ order. Likewise, ERNA-SARS2-503-GDura was generated by concatenation of antis_5p_SARS2_invert_pA (SEQ ID NO: 143), rcCDS_GDura (SEQ ID NO: 301), and antis_3p_SARS2_invert_IRES (SEQ ID NO: 147), while ERNA-SARS2-504-GDura was generated by concatenation of antis_5p_SARS2_invert_pA_HDVR (SEQ ID NO: 144), rcCDS_GDura (SEQ ID NO: 301), and antis_3p_SARS2_invert_IRES (SEQ ID NO: 147). This construction strategy is depicted in a simplified schematic in FIG. 19.

Four analogous sarbecovirus antisense encrypted RNA scaffolds were also generated for compatibility with the GL virus infection system (ERNA-SARS2-601 through ERNA-SARS2-604). For example, ERNA-SARS2-601-GDura was generated by concatenation of antis_5p_SARS2 (SEQ ID NO: 138), rcCDS_GDura (SEQ ID NO: 301), and antis_3p_SARS2GL (SEQ ID NO: 146) in 5′ to 3′ order. Similarly, ERNA-SARS2-602-GDura was generated by concatenation of antis_5p_SARS2GL_invert_pA_HDVR (SEQ ID NO: 141), rcCDS_GDura (SEQ ID NO: 301), and sense_3p_SARS2 (SEQ ID NO: 130) in 5′ to 3′ order. Likewise, ERNA-SARS2-603-GDura was generated by concatenation of antis_5p_SARS2GL_invert_pA (SEQ ID NO: 140), rcCDS_GDura (SEQ ID NO: 301), and antis_3p_SARS2_invert_IRES (SEQ ID NO: 147), while ERNA-SARS2-604-GDura was generated by concatenation of antis_5p_SARS2GL_invert_pA_HDVR (SEQ ID NO: 141), rcCDS_GDura (SEQ ID NO: 301), and antis_3p_SARS2_invert_IRES (SEQ ID NO: 147). As before, this construction strategy is depicted in a simplified schematic in FIG. 19.

Introduction of Additional Protein Payloads into Sarbecovirus Encrypted RNA Scaffolds

The above sarbecovirus encrypted RNA scaffolds described above for GDura as the polypeptide of interest were used as a basis to similarly encode alternative polypeptides of interest as listed in Table 5 or Table 10, including: EGFP, RLuc8, human IFN-β, human IFN-lambda1, human IFN-lambda3, mouse IFN-β, mouse IFN-lambda2, mouse IFN-lambda3, Syrian hamster IFN-β, or domestic ferret IFN-β.

For example, additional sarbecovirus sense encrypted RNA scaffolds encoding alternative polypeptides of interest were created by replacing CDS_GDura in ERNA-SARS2-001-GDura through ERNA-SARS2-011-GDura and in ERNA-SARS2-101-GDura through ERNA-SARS2-111-GDura with an alternative sense CDS selected from Table 5 or Table 10.

Sarbecovirus antisense encrypted RNA scaffolds encoding alternative polypeptides of interest were created by replacing rcCDS_GDura in ERNA-SARS2-501-GDura through ERNA-SARS2-504-GDura and in ERNA-SARS2-601-GDura through ERNA-SARS2-604-GDura with an alternative antisense CDS selected from Table 5 or Table 10. Following the convention of the previous nomenclature, “ERNA-SARS2-X-Z” indicates that sarbecovirus encrypted RNA Scaffold X encodes polypeptide of interest Z.

### DNA-Encoding of Sarbecovirus Encrypted RNA for In Vitro Production of RNA

A DNA-encoded construct for production of encrypted RNA via in vitro transcription was generated by cloning a sarbecovirus encrypted RNA cassette in between a T7 promoter on the 5′ end (Promoter_T7_Core, SEQ ID NO: 331) and a convenient 3′ BbsI restriction site (RE_BbsI_3p, SEQ ID NO: 336) within the MCS of the pUC19 vector (SapI was not used unlike in Example 1, as SapI cleaves within some sarbecovirus encrypted RNA sequences). This DNA-encoded construct is referred to as pAT202-X, where X is the sarbecovirus encrypted RNA scaffold. After linearization via BbsI digestion, this template can used to produce uncapped, 5′-triphosphorylated transcripts with standard in vitro transcription with four standard ribonucleotides (ATP, CTP, GTP, UTP) or 5′-capped transcripts (e.g. transcripts with Cap 1 structures generated via co-transcriptional capping by using CleanCap Reagent AU, TriLink BioTechnologies).

An alternative in vitro transcription template for generation of 5′-monophosphorylated sarbecovirus encrypted RNA was created similarly by cloning a sarbecovirus encrypted RNA cassette by concatenation of an alternative T7 promoter (Promoter_T7_AT, SEQ ID NO: 332), a sarbecovirus sense hammerhead ribozyme sequence (UTR_5p_SARS2_sense_HHRz, SEQ ID NO: 343), a sarbecovirus encrypted RNA scaffold, and a convenient 3′ BbsI restriction site (RE_BbsI_3p, SEQ ID NO: 336). For simplicity, this construct is referred to as pAT223-X, where X is the sarbecovirus encrypted RNA scaffold.

Similarly, pAT224-X was generated for compatibility with a co-transcriptional cap analog (such as CleanCap GG), by concatenation of an alternative T7 promoter (Promoter_T7_GG, SEQ ID NO: 334), a sarbecovirus sense hammerhead ribozyme sequence (UTR_5p_SARS2_sense_HHRz; SEQ ID NO: 343), a sarbecovirus encrypted RNA scaffold, and a convenient 3′ BbsI restriction site (RE_BbsI_3p, SEQ ID NO: 336).

### DNA-Encoding of Sarbecovirus Encrypted RNA for Intracellular Production of Encrypted RNA

A DNA-encoded sarbecovirus encrypted RNA cassette for use in mammalian cells was generated by cloning a sarbecovirus encrypted RNA scaffold in between a human cytomegalovirus promoter at the 5′ end (Promoter_CMV_IE2, SEQ ID NO: 337) and at the 3′ end by a concatenation of two contiguous sequences: (i) hepatitis delta virus antigenomic ribozyme (UTR_3p_HDVR_antigenomic, SEQ ID NO: 338), which was followed by; (ii) bovine growth hormone polyadenylation signal (UTR_3p_BGH_polyA, SEQ ID NO: 339), within the pUC19 cloning vector. For brevity, this vector is referred to as pAT101. Transfection of a mammalian cell line (e.g., BHK-21, A549, 293T) with pAT101 encoding an encrypted RNA construct leads to the RNA Polymerase II-driven production of a single-stranded 5′-capped encrypted RNA within the nucleus that is subsequently exported from the nucleus. In this way, an encrypted RNA can be delivered in DNA-vectored form with the resulting ssRNA transcript becoming accessible to the sarbecovirus replication complex outside of the nucleus. Following the convention of the previous nomenclature, pAT101-ERNA-SARS2-001-GDura would indicate that ERNA-SARS2-001-GDura was cloned into the central position within pAT101 for proper expression.

An alternative DNA-encoded sarbecovirus encrypted RNA cassette for use in mammalian cells was generated by cloning a sarbecovirus antisense encrypted RNA scaffold in between a human PolI promoter at the 5′ end (Promoter_PolI_human, SEQ ID NO: 353) and at the 3′ end by a concatenation of two contiguous sequences: (i) hepatitis delta virus antigenomic ribozyme (UTR_3p_HDVR_antigenomic, SEQ ID NO: 338), which was followed by; (ii) an enhanced Pol I terminator sequence (Terminator_PolI_mouse_enhanced, SEQ ID NO: 355), within the pUC19 cloning vector. For brevity, this vector is referred to as pAT401. An alternative construct was also developed, termed pAT402, which incorporated the same elements as pAT401 but omitted the 3′ antigenomic HDVR sequence.

Transfection of a mammalian cell line (e.g., BHK-21, A549 or 293T) with pAT401 or pAT402 encoding an encrypted RNA construct leads to the RNA Polymerase I-driven production of a single-stranded encrypted RNA within the nucleus that can be subsequently exported from the nucleus. In this way, an encrypted RNA can be delivered in DNA-vectored form with the resulting ssRNA transcript becoming accessible to the sarbecovirus replication complex outside of the nucleus. Following the previous nomenclature, pAT401-ERNA-SARS2-601-GDura would indicate that ERNA-SARS2-601-GDura was cloned into the central position within pAT401 for proper expression.

### Example 19: Activation of Sarbecovirus Encrypted RNAs by SARS-COV-2

### Generation-Limited Virus

A BSL-2 generation-limited (“GL” or “SARS2-GL”) system was utilized to conduct certain studies of encrypted RNA activation and efficacy at BSL-2 (since research with infectious SARS-COV-2 virus is currently only permitted within BSL-3 laboratories).

The GL virus was generated from a single plasmid BAC system that can launch coronavirus replication by transfecting permissive cells (such as Vero E6 and derivatives) with the BAC plasmid. Within the plasmid, a coronavirus genome cDNA is flanked on the 5′ end by a human cytomegalovirus promoter on the 3′ end by a bipartite cassette comprised of an antigenomic hepatitis delta virus ribozyme followed by a bovine growth hormone polyadenylation signal sequence. The cDNA contains several mutations with respect to the reference SARS-COV-2 sequence (NCBI GenBank ID: NC_045512.2), including: (i) deletion of ORF3a; (ii) deletion of E; (iii) modification of each of the TRS sequences from ACGAAC to CCGGA; (iv) introduction of a fluorescent protein reporter cassette at the 3′ end of the genome.

The GL virus requires ORF3a and E complementation for multi-round growth. To provide this, a line of Vero E6 cells expressing high levels of angiotensin converting enzyme-2 (ACE2) were transduced with a Tet-On lentiviral vector encoding three cassettes: (i) an inducible promoter with internal Tet operator sites that drives inducible expression of an ORF3a and E cDNA; (ii) a constitutive promoter driving production of a Tet activator that is only functional in the presence of doxycycline (or tetracycline); (iii) a constitutive promoter driving transcription of a gene conferring resistance to puromycin selection (an N-acetyltransferase). The Vero E6 cells harboring this transgene cassette (via lentiviral transduction) are termed Vero-E6-hACE2+ORF3a/E.

SARS-2 specific activation of protein expression encoded by encrypted RNA in Vero E6 hACE2 cells expressing ORF3a and E proteins

At 16 hours before infection, Vero-E6-hACE2+ORF3a/E cells were plated in 48-well plates at a density of 4×10⁴ cells/well in D10 supplemented to a final concentration of 0.2 μg/mL doxycycline (to induce expression of ORF3a & E) and 10 μg/mL puromycin.

At 0 hpi, the prepared cells were mock-infected (MOI-0) or at an MOI of 1 or 10 by incubating with a BSL-2 infection model of SARS-COV-2 (generation-limited SARS-COV-2 virus (SARS2-GL)) for 1 h at 37° C. in a humidified 5% CO₂ incubator. Immediately after the 1 h infection, cells were treated with 250 ng of an LMAX-formulated encrypted RNA or with a control uncapped mRNA encoding EGFP. Over the subsequent three days, culture media was collected daily (24 hpi, 48 hpi, 72 hpi) and assayed for secreted luciferase expression as described above.

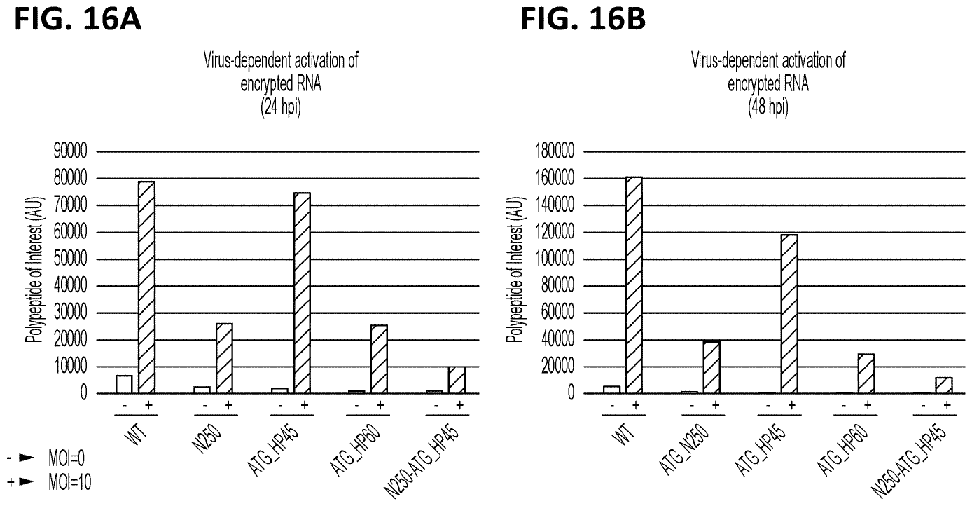

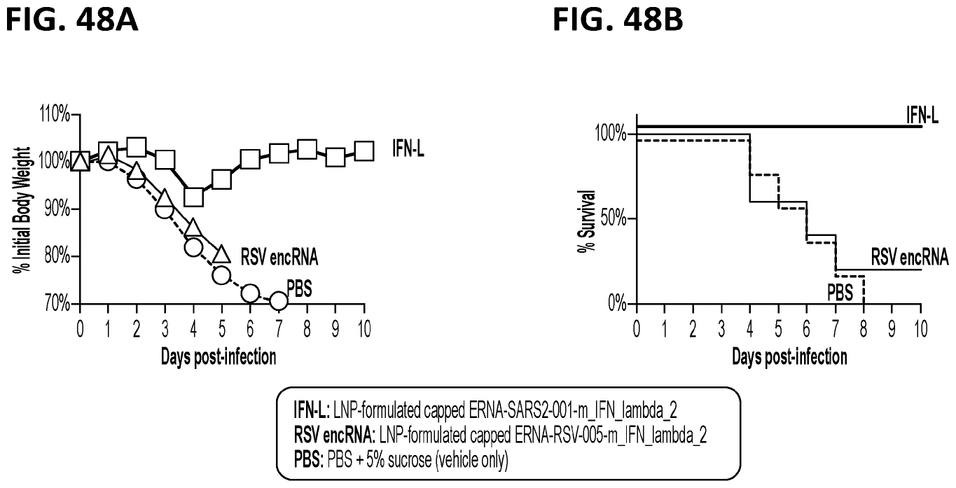

FIGS. 16A and 16B show that SARS-COV-2 infection can activate some sarbecovirus sense encrypted RNAs. Vero-E6-hACE2+ORF3a/E cells were treated with a sarbecovirus sense encrypted RNA encoding GDura as the polypeptide of interest, as described in the preceding paragraph. The treated cells were then infected with SARS2-GL at an MOI of 10, and the level of GDura was measured at both 24 hours (FIG. 16A) and 48 hours post-infection (FIG. 16B). Multiple sarbecovirus sense encrypted RNA constructs were tested for activation in this study: ERNA-SARS2-101-GDura (labelled “WT”), ERNA-SARS2-102-GDura (labelled “N250”), ERNA-SARS2-109-GDura (labelled “ATG_HP45”), ERNA-SARS2-110-GDura (labelled “ATG_HP60”), and ERNA-SARS2-105-GDura (labelled “N250-ATG_HP45”). For each tested encrypted RNA, FIG. 16A shows levels of the polypeptide of interest at 24 hours post-infection with SARS2-GL and FIG. 16B shows levels of the polypeptide of interest at 48 hours post-infection with SARS2-GL, compared to the level of activation of interest in the absence of sarbecovirus infection.

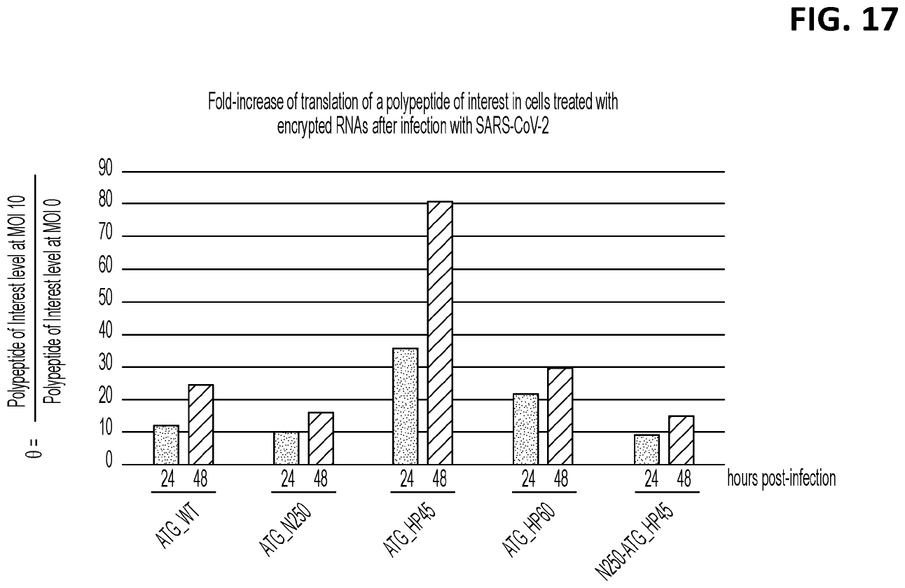

FIG. 17 presents these results as the quotient of the level of polypeptide of interest translation in SARS2-GL infected cells divided by the level of polypeptide of interest translation in uninfected cells: 0=LEVEL_ON/LEVEL_OFF. In all of these examples, θ>1 (i.e., translation of the polypeptide of interest was always increased by SARS2-GL infection in these examples). Importantly, certain versions of the encrypted RNA have a greater 0 than others; e.g. some cassettes incorporating ATG_HP45 have a substantially increased 0 (reaching 80 at 48 hpi). As a quotient, 0 can be increased by either increasing the LEVEL_ON dividend (greater protein levels in activated state, higher peak expression) or by decreasing the background “leakiness” in the uninfected encrypted state (decreasing the LEVEL_OFF divisor). By comparing FIG. 17 and FIG. 16, it is apparent that modification of the encrypted RNA from WT to ATG_HP45 slightly decreased LEVEL_ON, but dramatically decreased LEVEL_OFF (the background leakiness).

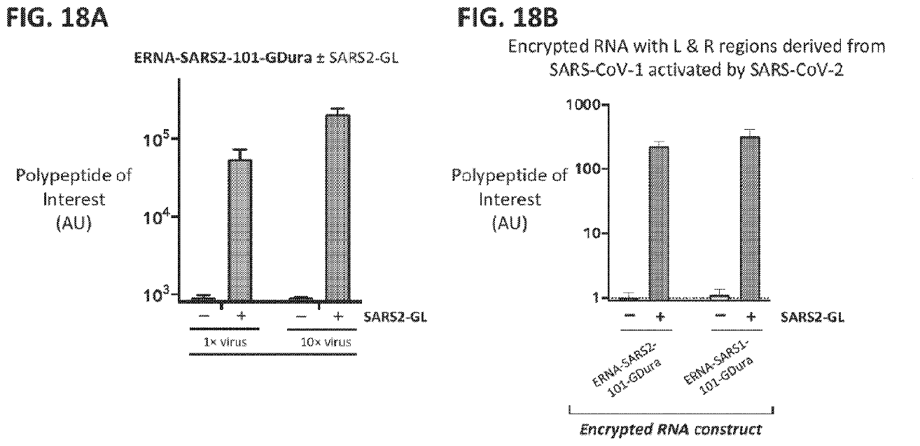

FIG. 18A shows that encrypted RNA activation can be dependent on the dose of a translation activator, which in this case is provided by infection with SARS2-GL at an MOI of 1 (indicated as “1× virus”) or 10 (indicated as “10× virus”). In the absence of infection, ERNA-SARS2-101-GDura produces approximately background levels of the polypeptide of interest (GDura) in Vero-E6-hACE2+ORF3a/E cells. SARS2-GL infection increased translation of the polypeptide of interest in a dose-dependent manner: by ~1.5 log with “1× virus” and ~2.5 log with “10× virus”.

FIG. 18B shows that a sarbecovirus encrypted RNA can also be developed using L and R regions that are derived from SARS-COV-1. A sarebcovirus encrypted RNA (ERNA-SARS1-101-GDura) built from L and R regions derived from SARS-COV-1 was LMAX-LNP-formulated and used to treat Vero-E6-hACE2+ORF3a/E cells. A parallel Vero-E6-hACE2+ORF3a/E culture was treated with LMAX-LNP-formulated ERNA-SARS2-101-GDura. Both treated cultures were also infected with SARS-COV-2 (GL) at an MOI of 1.

Notably, treatment with the SARS-COV-1 derived encrypted RNA resulted in an activation level of the polypeptide of interest (GDura) comparable to or higher than that measured in cells treated with the SARS-COV-2 derived encrypted RNA during SARS-COV-2 GL infection.

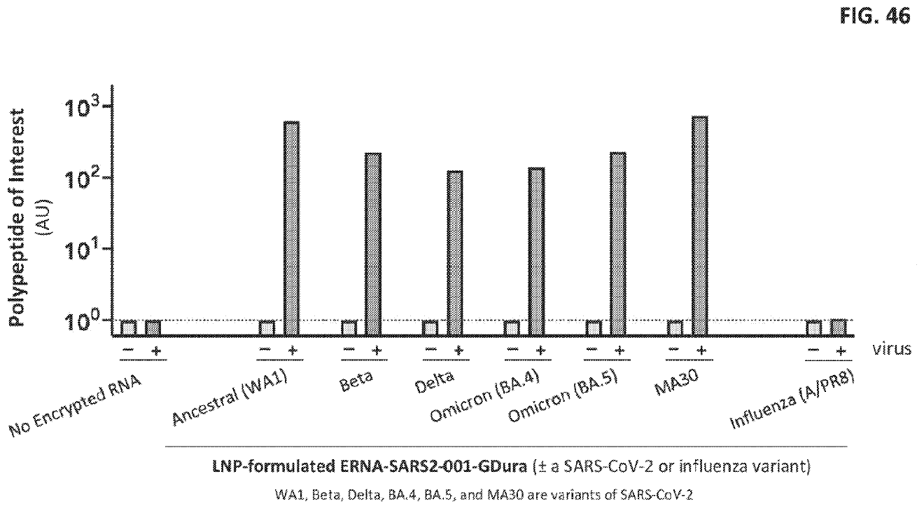

The SARS-COV-1 genome has ~20% sequence divergence from the SARS-CoV-2 genome, yet a SARS-COV-1 based encrypted RNA is substantially activated by SARS-COV-2. Together with FIG. 46 below, this further indicates that some sarbecovirus encrypted RNAs can be activated by a diverse set of sarbecoviruses.

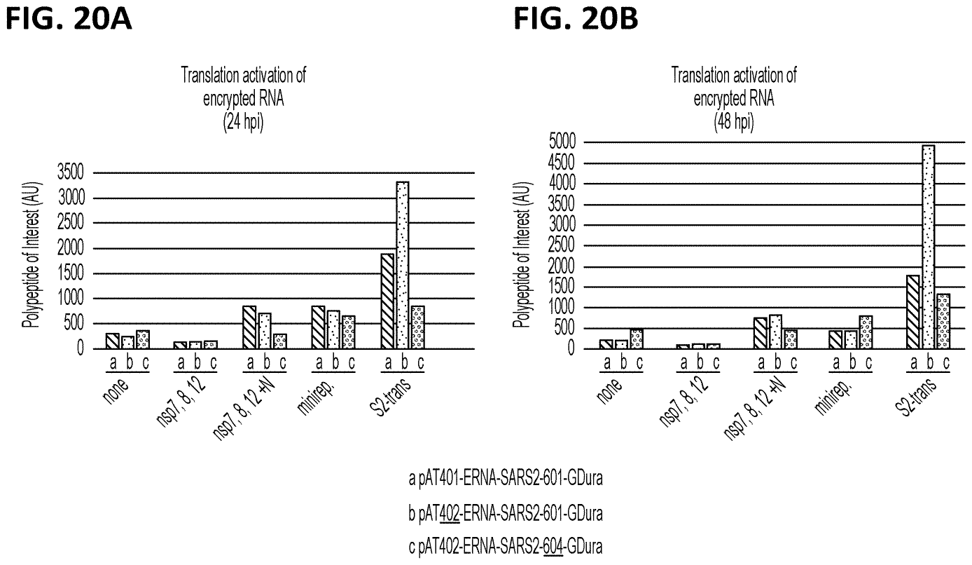

FIG. 20 shows that translation activators of sarbecovirus antisense encrypted RNA can be provided by: (i) viral infection initiated by transfection of a DNA encoding the virus genome, or by (ii) providing expression plasmids that produce polypeptides, which together comprise a translation activator of a sarbecovirus encrypted RNA. In this experiment, cells were transfected with a DNA-encoded vector producing a sarbecovirus encrypted RNA and one of 5 additional sets of transfection conditions: (i) no additional plasmids (“none”); (ii) plasmids producing SARS-COV-2 nsp7, nsp8, nsp12 polypeptides; (iii) plasmids producing SARS-COV-2 nsp7, nsp8, nsp12, and Nucleoprotein (N) polypeptides; (iv) a multigenic BAC expression plasmid which drives SARS-COV-2 orf1ab production from a constitutive minimal HCMV IE2 promoter and separately drives SARS-CoV-2 Nucleoprotein (N) production from an EF1a promoter (“minirep”); (v) a SARS-COV-2 BAC which produces a SARS-COV-2 genome competent for orf1ab production but deficient for all structural proteins except N (“S2-trans”).

The following sarbecovirus antisense encrypted RNAs were used: (a) pAT401-ERNA-SARS2-601-GDura; (b) pAT402-ERNA-SARS2-601-GDura; (c) pAT402-ERNA-SARS2-604-GDura. The key differences among these encrypted RNAs were the removal of the 3′ HDVR sequences in (b) relative to (a), and the use of a different antisense encrypted RNA scaffold in (b) relative to (c).

For each of the encrypted RNA constructs tested, provision of nsp7, nsp8, nsp12, and N was sufficient to activate the encrypted RNAs. The best performing and strongest translation activator was a simulated viral infection generated by transfection with the SARS2-GL virus BAC encoding all non-structural proteins and Nucleoprotein (N). Thus, infection or complementation with key viral proteins can activate translation of a sarbecovirus encrypted RNA.

The potential for activation of encrypted RNA when the RNA sequences are delivered in circular RNA form was also tested. At 0 hpi, Vero-E6-hACE2+ORF3a/E cells were mock-infected (MOI=0) or infected at an MOI of 10 by incubating with a BSL-2 infection model of SARS-COV-2 (SARS2-GL) for 1 h at 37° C. in a humidified 5% CO₂ incubator. Immediately after the 1 h infection, cells were treated with ~250 ng of LMAX-LNP formulated encrypted RNA (with GDura as the polypeptide of interest) that was prepared with circularized or non-circular RNA form as described in Example 7 (non-circular RNA contains the flanking self-splicing intron sequences). Over the subsequent three days, culture media was collected daily (24 hpi, 48 hpi, 72 hpi) and assayed for secreted luciferase expression as described above.

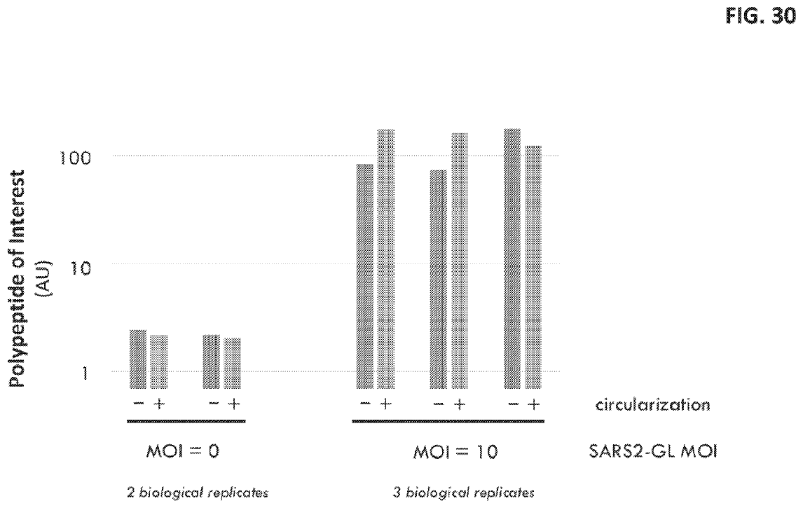

FIG. 30 demonstrates that a circular encrypted RNA (containing the ERNA-SARS2-101-GDura sequence) retains the ability to be activated by viral infection of a cell. Uninfected Vero-E6-hACE2+ORF3a/E cells treated with linear or circular encrypted RNAs had approximately background levels of translation of the polypeptide of interest at 72 hpi. In contrast, in Vero-E6-hACE2+ORF3a/E cells infected with SARS2-GL, treatment of the cells with a circular encrypted RNA or a linear encrypted RNA (with flanking ends) resulted in increased translation of the polypeptide of interest (approximately 100× or 2 log above the level in uninfected cells).

In a further study, nucleoside-modified sarbecovirus encrypted RNAs were prepared. Sarbecovirus encrypted RNAs were prepared using ERNA-SARS2-002 scaffold (N250 insertion relative to −001 “WT” scaffold) and encoded GDura (ERNA-SARS2-002-GDura) or human IFN-β (ERNA-SARS2-002-hu_IFNB).

Encrypted RNAs were prepared via high temperature in vitro transcription (50° C.) and capped with a Cap1 via co-transcriptional capping (CleanCap) as described above in Example 6, using three different nucleotide pools: (i) “100% U”, an equimolar solution of ATP, CTP, GTP, and UTP during in vitro transcription; (ii) “70% Ψ”, analogous to the previous but the ‘UTP’ pool was comprised of a binary mixture of 30% UTP and 70% pseudouridine triphosphate (percentages are % by mol); (iii) “100% m1Ψ”, where the ‘UTP pool’ was comprised of about 100% N1-methylpseudouridine (m1Ψ) triphosphate.

The six different encrypted RNAs were formulated into LMAX-LNPs and used to treat A549 Dual cells. A549 Dual cells have a genome with an endogenous secreted luciferase under control of a minimal ISG54 promoter with 5 interferon sensitive response elements (the “ISRE promoter”). At 24 h after treatment, the total secreted level of luciferase (which can be comprised of endogenous production from the ISRE promoter in these cells driving luciferase expression or exogenous production from any leaky activation of the encrypted RNAs) was quantified as described above. Notably, activation of the ISRE promoter can occur due to recognition by cellular receptors (e.g. RIG-I or MDA5) of pathogen-associated molecular patterns (“PAMPs”), such as double-stranded RNA (dsRNA) or 5′-triphosphorylated dsRNA, on RNAs, which can activate cellular innate immune pathways. For the encrypted RNAs encoding human IFN-β as the polypeptide of interest, the luciferase pool reports on the level of innate immune system activation from treatment with an RNA as well as any ‘leaky’ translation of the encoded human IFN-β protein, which would activate luciferase secretion from the cell line (via the ISRE promoter). Similarly, for the encrypted RNAs encoding GDura as the polypeptide of interest, the total luciferase pool can have contributions from innate immune activation as well as direct ‘leaky’ translation of GDura from the encrypted RNA.

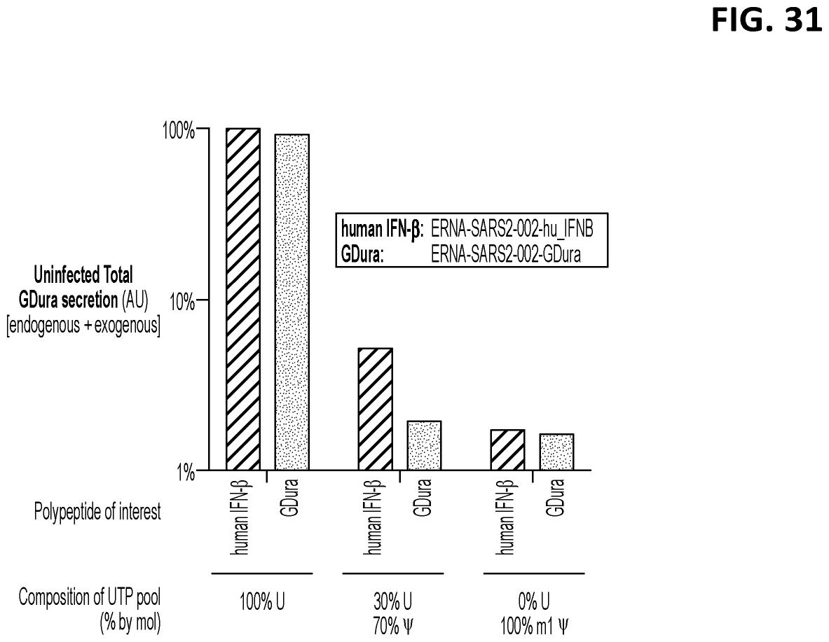

FIG. 31 shows that, in the absence of a translation activator, treatment of A549 Dual cells with nucleoside-modified encrypted RNAs can result in lower background levels of immunogenicity or encrypted RNA activation than treatment of cells with corresponding non-nucleoside modified encrypted RNAs. For example, in the absence of a translation activator, treatment of A549 Dual cells with a nucleoside-modified sarbecovirus encrypted RNA incorporating the 70% nucleotide pools resulted in a level of total luciferase secretion that was ~10% of the level measured in cells analogously treated with a corresponding sarbecovirus encrypted RNA incorporating the 100% U nucleotide pools. In addition, in the absence of a translation activator, treatment of A549 Dual cells with a nucleoside-modified sarbecovirus encrypted RNA incorporating the 100% m1P nucleotide pools resulted in a level of total luciferase secretion that was approximately ~2% of the level measured in cells analogously treated with a corresponding sarbecovirus encrypted RNA incorporating the 100% U nucleotide pools. Thus, nucleoside modification of an encrypted RNA can reduce the immunogenicity of the encrypted RNA or background translation of the polypeptide of interest in the absence of a translation activator.

The effect of incorporating modified nucleotides on the activation of a sarbecovirus encrypted RNA was also tested in Vero-E6-hACE2+ORF3a/E cells infected with SARS2-GL. Encrypted RNAs were produced by in vitro transcription using a thermostable T7 RNA polymerase, as described above in Example 6 using CleanCap AU (Trilink, cat. No. N-7114) for co-translational capping. Four different types of modified nucleotides were used for incorporation into ERNA-SARS2-101-GDura scaffold: (i) N⁶-methyladenosine (“m6a”); (ii) pseudouridine (“Ψ”), (iii) N1-methylpseudouridine (m¹Ψ), (iv) or 5-methoxyuridine (5moU). The rate of incorporation was controlled by changing the percentage of modified nucleotides to the total amount of adenosine or uridine (percentages are mol %) in the IVT reaction mix. IVT RNAs with modified nucleotides were formulated into LNPs, as described in Example 9, and activation by SARS2-GL was measured via luciferase assay.

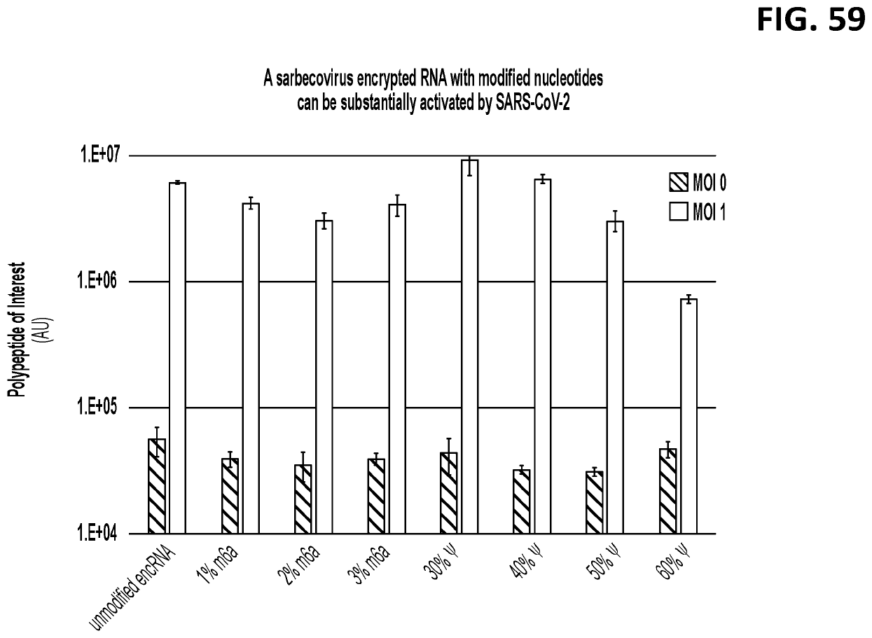

FIG. 59 shows that a sarbecovirus encrypted RNA, ERNA-SARS2-101-GDura, incorporating modified nucleotides can be substantially activated by SARS2-GL. The figure shows that incorporation of 1-3% N6-methyladenosine had no significant effect on the activation of a sarbecovirus encrypted RNA when the cells were infected with SARS-COV-2 GL. Further, when ERNA-SARS2-101-GDura was formulated with 30-60% of uridine nucleotides modified to pseudouridine, the encrypted RNA was activated similarly or up to approximately 1.5-fold higher than a matched ERNA-SARS2-101-GDura construct formulated with 100% unmodified U in response. Similarly, when ERNA-SARS2-101-GDura was formulated with 30-60% of uridine nucleotides modified to N1-methylpseudouridine or 5-methoxyuridine, the encrypted RNA was activated approximately similarly or higher than a matched ERNA-SARS2-101-GDura construct formulated with 100% unmodified U.

Importantly, increasing the percentage of modified U nucleotides above ~40% significantly decreased activation of the sarbecovirus encrypted RNAs in response to sarbecovirus infection of treated cells.

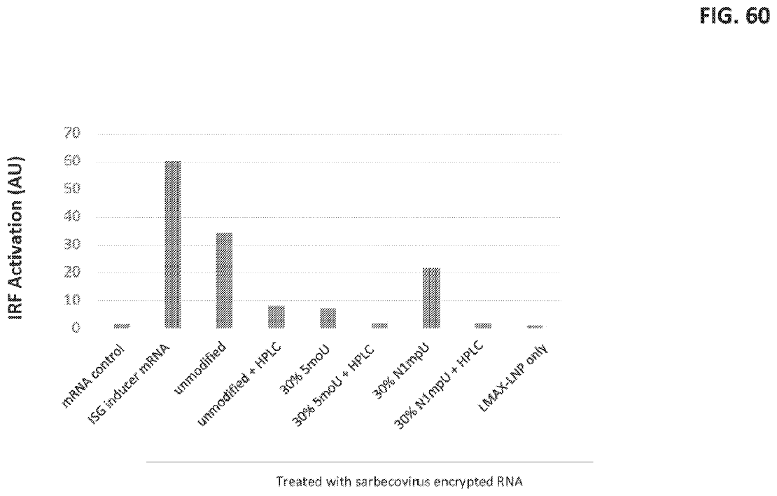

FIG. 60 shows that the immunogenicity of an LMAX-LNP formulated sarbecovirus encrypted RNA (ERNA-SARS2-101-GDura) in human A549 Dual cells can be substantially decreased by using modified nucleotides, including additional steps of purification after IVT (i.e. HPLC), or by combining additional purification of the RNA with the use of modified nucleotides. In this study, IVT of the sarbecovirus encrypted RNA was performed as described in Example 6, HPLC purification was performed as described in Example 7, and immunogenicity was quantified as in Example 19. Notably, modifying ~30% of uridine nucleotides to 5-methoxyuridine and performing HPLC was sufficient to reduce encrypted RNA immunogenicity to near background levels (i.e. the levels generated by the LMAX-LNP formulation alone).

In a further study, the impact of 5′-capping on the level of encrypted RNA activation was measured. RNA was prepared using either an ERNA-SARS2-002-GDura scaffold (which has an N250 insertion relative to “WT”-001) or ERNA-SARS2-102-GDura (TRS2 relative to ‘002’). Capped encrypted RNA was prepared via in vitro transcription using a thermostable T7 RNA polymerase and capped with Cap1 via co-transcriptional capping (CleanCap) as described above in Example 6. Uncapped encrypted RNA was prepared similarly, except the CleanCap analog was omitted from the IVT reaction.

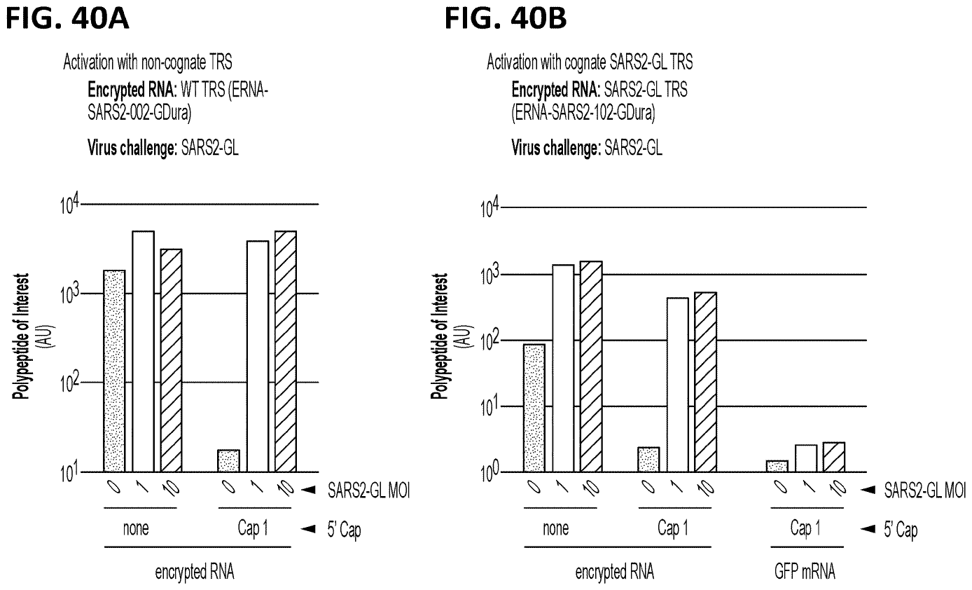

At 0 hpi, Vero-E6-hACE2+ORF3a/E cells were mock-infected (MOI-0) or infected at an MOI of 1 or 10 by inoculation with SARS2-GL for 1 h at 37° C. in a humidified 5% CO₂ incubator. Immediately after the 1 h infection, cells were treated with ~250 ng of LMAX-LNP formulated encrypted RNA (capped or uncapped species described above). Over the subsequent two days, culture media was collected daily (24 hpi, 48 hpi) and assayed for production of the GDura polypeptide of interest (secreted luciferase expression), as described above. The results of this study are shown in FIGS. 40A-40B.

As FIGS. 40A-40B show, in cells treated with a sarbecovirus sense encrypted RNA, 5′ capping (or lack thereof) of the encrypted RNA can strongly affect the background level of translation of the polypeptide of interest in the absence of a translation activator (e.g. in the absence of viral infection). For example, in the absence of SARS2-GL viral infection, cells treated with a 5′-capped encrypted RNA have significantly lower levels of translation of the polypeptide of interest (GDura) than cells treated with the corresponding uncapped sarbecovirus encrypted RNA (i.e., the level of background translation of GDura in uninfected cells treated with the capped encrypted RNA is ~5% of the level of GDura in uninfected cells treated with the uncapped encrypted RNA). Conversely, in cells infected with SARS2-GL (and thereby provided with a translation activator), treatment with 5′-capped encrypted RNAs or 5′-uncapped encrypted RNAs resulted in comparably high levels of translation of the polypeptide of interest, while cells treated with a negative control (GFP) mRNA had low levels of luciferase activity.

FIG. 40A further shows that the TRS sequences within a sarbecovirus encrypted RNA can also be modified (e.g. from ACGAAC to CCGGAT) if they are internally self-consistent, without ablating translation activation or substantially influencing the background translation of the polypeptide of interest. FIG. 40A shows that SARS2-GL can substantially activate a sarbecovirus encrypted RNA possessing a different TRS (“non-cognate TRS”) than the viral genome. FIG. 40B shows that SARS2-GL can substantially activate a sarbecovirus encrypted RNA possessing the same TRS (“cognate TRS”) as the viral genome.

### Example 20: Antiviral Activity of Sarbecovirus Encrypted RNA In Vitro Using a BSL-2 GL SARS-COV-2 System

At 16 hours before infection, Vero-E6-hACE2+ORF3a/E cells were plated in 24-well plates at a density of 7×10⁴ cells/well in D10 supplemented to a final concentration of 0.2 μg/mL doxycycline (to induce expression of ORF3a & E) and 10 μg/mL puromycin (to maintain selection for the inducible ORF3a/E cassette).

At 0 hpi, the prepared cells were mock-infected (MOI-0) or infected with SARS-GL an MOI of 0.1 or 1.0 by incubating with a BSL-2 infection model of SARS-COV-2 (generation-limited SARS-COV-2 virus (SARS2-GL)) for 1 h at 37° C. in a humidified 5% CO₂ incubator. Immediately after infection, cells in each well were treated with 6-500 ng of LMAX-LNP formulated encrypted RNA or with control uncapped mRNA encoding EGFP. At 72 hpi, virus-containing culture media was collected for viral RNA isolation, and the SARS2-GL viral titers (as vg/mL) subsequently quantified by RT-qPCR as described above.

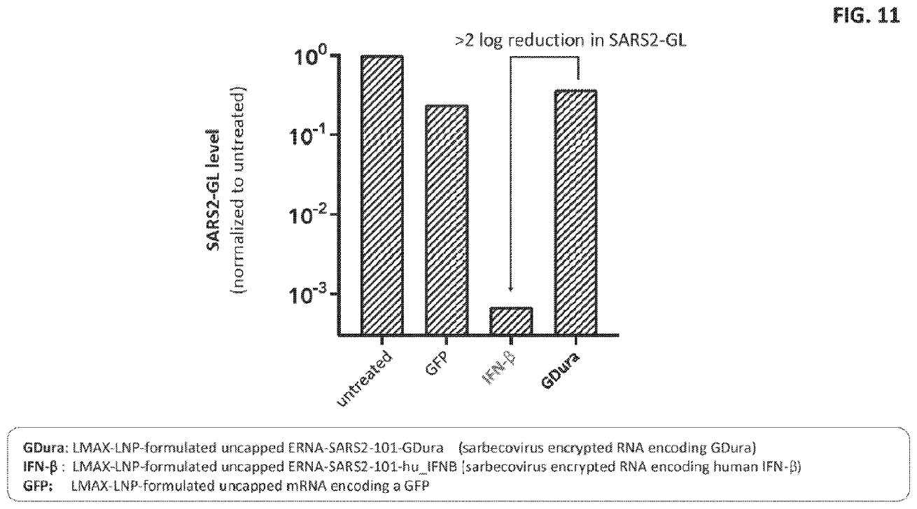

FIG. 11 demonstrates the antiviral efficacy of a therapeutic sarbecovirus encrypted RNA formulated into an LMAX-LNP. Treatment of Vero-E6-hACE2+ORF3a/E cells with a therapeutic sarbecovirus encrypted RNA encoding human IFN-β (500 ng of LMAX-LNP formulated uncapped ERNA-SARS2-101-hu_IFNB) elicited an approximately 3 log reduction in the SARS2-GL virus level compared to the virus level in untreated cells, and a more than 2 log reduction in the SARS2-GL virus level compared to the virus level in cells that received a control sarbecovirus encrypted RNA formulated analogously (500 ng of LMAX-LNP-formulated uncapped ERNA-SARS2-101-GDura) or a control mRNA (500 ng of LMAX-LNP-formulated uncapped mRNA encoding a GFP).

To test the specificity of a therapeutic sarbecovirus encrypted RNA for a translation activator provided by sarbecoviruses, human A549-RIGI-KO cells were infected with a non-sarbecovirus (influenza A/PR8 at MOI 0.01). One hour later, the cells were treated with one of: 250 ng of an LMAX-LNP-formulated therapeutic sarbecovirus encrypted RNA (uncapped ERNA-SARS2-101-hu_IFNB), 250 ng of a control LMAX-LNP-formulated sarbecovirus encrypted RNA (uncapped ERNA-SARS2-101-GDura), or LMAX-LNP alone (no RNA). Two days after treatment, the amount of influenza virus was titrated by focus forming unit assay.

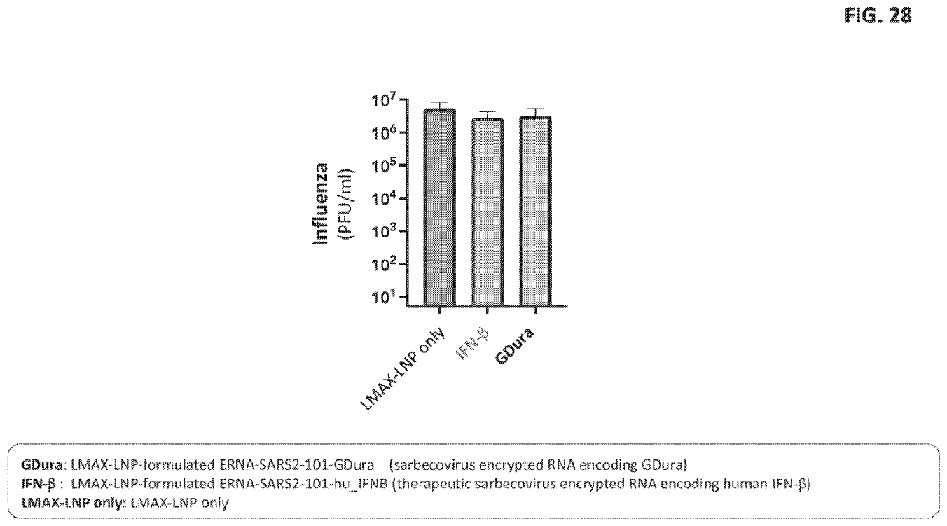

As shown in FIG. 28, treatment of A549-RIGI-KO cells with the therapeutic sarbecovirus encrypted RNA (ERNA-SARS2-101-hu_IFNB) or a control sarbecovirus encrypted RNA (ERNA-SARS2-101-GDura) did not substantially reduce influenza replication in the cells, indicating the specificity of a therapeutic sarbecovirus encrypted RNA for a translation activator provided by sarbecovirus infection.

Notably, in Example 16, an influenza encrypted RNA encoding the same polypeptide of interest (human interferon-beta) was shown to substantially reduce influenza A/PR8 viral levels, during which the influenza encrypted RNA is substantially activated. FIG. 28 therefore shows that the activity of a therapeutic polypeptide of interest can be limited—e.g. to enable the targeted prophylaxis or treatment of viruses of a specific viral species—by encoding the therapeutic polypeptide in an encrypted RNA cassette.

In addition, the same sarbecovirus encrypted RNAs (ERNA-SARS2-101-hu_IFNB and ERNA-SARS2-101-GDura) were tested against a second non-sarbecovirus: human parainfluenza virus type-3 (HPIV3). In this experiment, parallel cultures of Vero-E6-hACE2+ORF3a/E cells were each treated with 500 ng total of mixtures of sarbecovirus encrypted RNAs formulated into LMAX-LNPs. The following four mixtures were formulated as LMAX-LNPs: 100% ERNA-SARS2-101-hu_IFNB; or 100% ERNA-SARS2-101-GDura; or a mixture of 10% ERNA-SARS2-101-hu_IFNB and 90% ERNA-SARS2-101-GDura; or no encrypted RNA. (Percentages here are mass percentages which is about equal to % by mol, as the length of the encrypted RNAs are similar). Parallel cell cultures were treated with one of these four mixtures and then infected 16 hours later with HPIV3. No substantial difference was measured between HPIV3 replication in cells treated with a therapeutic sarbecovirus encrypted RNA (the LMAX-LNPs containing 100% ERNA-SARS2-101-hu_IFNB) and cells treated with a control encrypted RNA mixture (e.g., the LMAX-LNPs containing 100% ERNA-SARS2-101-GDura), as measured by RT-qPCR. In contrast, when parallel analogously treated Vero-E6-hACE2+ORF3a/E cells were infected, 16 hours after treatment, with SARS2-GL (instead of HPIV3), the cells treated with a therapeutic sarbecovirus encrypted RNA (the LMAX-LNPs containing 100% ERNA-SARS2-101-hu_IFNB) had SARS2-GL viral levels that were approximately 10-100× lower than in untreated cells or in cells treated with a control sarbecovirus encrypted RNA (LMAX-LNPs containing 100% ERNA-SARS2-101-GDura), as measured by RT-qPCR.

Collectively, FIG. 28 and the additional experiments with HPIV3 and SARS2-GL in Vero-E6-hACE2+ORF3a/E cells show that while an LMAX-LNP-formulated therapeutic sarbecovirus encrypted RNA is highly effective at inhibiting a sarbecovirus, the therapeutic sarbecovirus encrypted RNA does not have substantial efficacy against non-sarbecoviruses such as influenza or HPIV3.

To additionally test whether different therapeutic proteins (e.g. beyond interferon proteins) could confer efficacy when encoded into the sarbecovirus encrypted RNA scaffold, the antiviral efficacy of a sarbecovirus encrypted RNA encoding a truncated SARS-COV-2 Spike protein [ERNA-SARS2-101-SARSSpikeM, where SARSSpikeM represents the CDS of CDS_SARS_Spike_trunctation (SEQ ID NO: 300)] was tested. The mutated SARS Spike protein monomers lack an essential extracellular domain but can interact with functional Spike protein monomers encoded by SARS-COV-2 to form defective trimeric Spike proteins and can thereby reduce the number of functional Spike proteins available to new SARS-COV-2 viral particles produced during viral replication in a treated cell. Thus, cells treated with an encrypted RNA encoding a truncated SARS-COV-2 Spike protein and infected with SARS-COV-2 virus, are expected to produce fewer functional SARS-COV-2 viral particles than untreated cells infected by the virus, resulting in a reduction in SARS-COV-2 virus level.

The antiviral efficacy of a therapeutic sarbecovirus encrypted RNA encoding the truncated Spike protein was tested by formulating the encrypted RNA into an LMAX-LNP. Treatment of Vero-E6-hACE2+ORF3a/E cells with the LMAX-LNP formulated therapeutic sarbecovirus encrypted RNA encoding the truncated SARS2 Spike protein (20 ng of LMAX-LNP formulated capped ERNA-SARS2-101-SARSSpikeM) elicited an approximately 4-fold reduction in the SARS2-GL virus level compared to the virus level in untreated cells or in cells that received a control sarbecovirus encrypted RNA formulated analogously (20 ng of LMAX-LNP-formulated capped ERNA-SARS2-101-GDura).

### Efficacy of a DNA-Encoded Sarbecovirus Encrypted RNA

DNA-encoded sarbecovirus encrypted RNA cassettes were developed using the lentiviral vector pLVG07 (described in Example 11 above). Two representative lentiviral transfer vectors were developed using the pLVG07 system: (i) pLVG07-ERNA-SARS1-101-GDura and (ii) pLVG07-ERNA-SARS1-101-hu_IFNB. The provirus (proviral) sequence of pLVG07-ERNA-SARS1-101-GDura is listed as SEQ ID NO: 417; the proviral sequence of pLVG07-ERNA-SARS1-101-hu_IFNB is listed as SEQ ID NO: 418 to accurately describe the position and orientation of each sarbecovirus encrypted RNA within their lentiviral transfer vector. The sequence of ERNA-SARS1-GDura is SEQ ID NO: 416. Preparation of LVG07-ERNA-SARS1-GDura and LVG07-ERNA-SARS1-hu_IFNB lentivirus was performed using the method described above (Example 12).

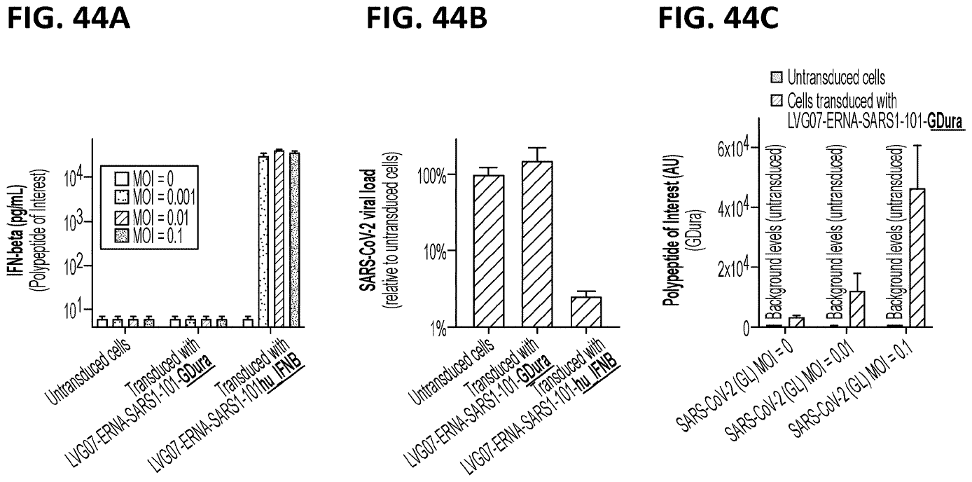

FIG. 44A and FIG. 44B, taken together, demonstrate the antiviral efficacy of a DNA-encoded sarbecovirus encrypted RNA. Vero hACE2 cells were transduced with LVG07-ERNA-SARS1-101-GDura or LVG07-ERNA-SARS1-101-hu_IFNB preparations. After transduction, cells were selected using puromycin at a final concentration of 20 mcg/mL. After puromycin selection, cells were expanded for approximately 14 days and plated on 24-well plates for subsequent infection with SARS2-GL.

To perform the infection, Vero hACE2, Vero hACE2 LVG07-ERNA-SARS1-101-GDura, or Vero hACE2 LVG07-ERNA-SARS1-101-huIFNb were plated on 24-well plates at a concentration of 10⁵ cells/well. At 18 hours after plating, cells were treated with 10 ng/cm² mRNA-LNP encoding Sars2-E and Sars2-ORF3a proteins (described above) to provide these proteins in trans and to enable proliferation of SARS2-GL virus in Vero hACE2 cells.

At 6 hours after LNP treatment, cells were infected with SARS2-GL at MOI 0.1, 0.01, 0.001 or 0, according to previously described protocol of infection of Vero cells. At 1 hour after adding virus, the infection media was aspirated and fresh D10 media was added. At 72 h post-infection, supernatants were collected and clarified from the cell debris by centrifugation. A sample of 140 mcL of collected media was used to isolate vRNA using QIAamp Viral DNA kit (Qiagen, cat no. 52906) according to the manufacturer's protocol and the SARS2-GL viral titers (as vg/mL) subsequently quantified by RT-qPCR as described above. Concentrations of secreted human IFN-beta were measured with LumiKine Xpress hIFN-b 2.0 Kit (InvivoGen, cat no. luex-hifnbv2).

FIG. 44A demonstrates that DNA-encoded encrypted RNAs can be delivered to cells and activated by viral infection weeks after delivery (>14 days). We observed significant activation and production of human IFN-beta from cells transduced with LVG07-ERNA-SARS1-101-hu_IFNB and infected with SARS2-GL according to the results of detection of human IFN-beta secreted into the culture media (>1E4 μg/mL IFN-beta). In contrast, untreated cells or cells transduced with LVG07-ERNA-SARS1-101-GDura showed no detectable levels of IFN-beta when infected with SARS2-GL or left uninfected. Moreover, IFN-beta secretion from LVG07-ERNA-SARS1-101-hu_IFNB transduced cells was dependent on sarbecoviral infection to activate the encrypted RNA- and levels of secreted IFN-beta were significant (>100 μg/mL) and detectable over the range of MOIs tested (0.01-1.0).

FIG. 44B shows that LVG07-ERNA-SARS1-101-hu_IFNB treatment of cells was effective at inhibiting viral infection, while treatment of cells with an analogous DNA-encoded encrypted RNA (LVG07-ERNA-SARS1-GDura) not encoding a therapeutic polypeptide was not effective at preventing virus replication. Levels of SARS-COV-2 (SARS-2 GL) were reduced about 1.5 log 10 (30×) when the DNA-encoded encrypted RNA encoded a therapeutic polypeptide of interest (IFN-beta) versus a non-therapeutic polypeptide of interest. Additionally, LVG07-ERNA-SARS1-GDura treated cells propagated virus to the same level as untreated cells, indicating that the antiviral efficacy of encrypted RNA treatment was specific to the polypeptide of interest.

Additionally, the specificity of a DNA-encoded sarbecovirus encrypted RNA efficacy against sarbecoviruses (and not untargeted viruses, such as influenza) was evaluated. A549 cells were transduced with LVG07-ERNA-SARS1-101-GDura or LVG07-ERNA-SARS1-101-hu_IFNB and selected with puromycin at final concentration 2 mcg/mL. After puromycin selection cells were expanded and plated on 24-well plates for infection with IAV/Puerto Rico/8/1934 (H1N1).

A549, A549 LVG07-ERNA-SARS1-101-GDura, A549 LVG07-ERNA-SARS1-101-hu_IFNB cells were plated on 24-well plate at a concentration of 1.2E5 cells/well. At 18 hours after plating, cells were infected with IAV/Puerto Rico/8/1934 (H1N1) at MOIs of 0.01, 0.001 or 0, according to the above methods. At 2 hours after adding virus, infection media was aspirated and fresh 293 expression medium (Gibco, ct no. 12338018) was added.

At 48 h post-infection supernatants were collected and clarified from the cell debris by centrifugation. No significant difference of IAV/Puerto Rico/8/1934 (H1N1) titers in experimental samples were observed after detecting with plaque assay according to the previously described protocol (data not shown).

To confirm that the encrypted RNA cassette was indeed functional after integration in an immunocompetent cells line, the level of activation of encrypted payload expression in A549 LVG07-ERNA-SARS1-101-GDura cells, as quantified by GDura secretion.

A549 LVG07-ERNA-SARS1-101-GDura were plated on 24-well plate at 1.2E5 cells/well. At 18 hours after plating, cells were treated with 10 ng/cm² LNP encoding Sars2-E, Sars2-ORF3a, TMPRSS2 and ACE2 proteins to provide these proteins in trans and to enable infection and proliferation of SARS2-GL virus in A549 cells. At 6 hours after LNP treatment, cells were infected with SARS2-GL at MOI 1, 0.1, and 0, according to previously described protocol of infection of Vero cells. At 1 hour after adding virus infection media was aspirated and fresh D10 media was added. At 72 hours after infection, supernatants were collected and a bioluminescent signal of expressed GDura was measured according to the previously described protocol.

FIG. 44C shows that immunocompetent cells (A549) can be effectively treated by transduction with a lentiviral vector encoding a DNA-encoded encrypted RNA, with the cassette persisting weeks after delivery (>14 days). Secretion of GDura from the activated encrypted RNA was proportional to the MOI of infection.

### Example 21: Antiviral Activity of a Therapeutic Sarbecovirus Encrypted RNA In Vitro Using Infectious Sarebcovirus Isolates in a BSL-3 Laboratory

A therapeutic sarbecovirus encrypted RNA (ERNA-SARS2-101-hu_IFNB), a control non-therapeutic sarbecovirus encrypted RNA (ERNA-SARS2-101-GDura), and a control mRNA encoding a GFP were formulated into LNPs following the approach in Example 9. Vero E6, Vero-hACE2-TMPRSS2, or Huh7 cells were treated with either the LNP-formulated therapeutic sarbecovirus encrypted RNA, the LNP-formulated non-therapeutic sarbecovirus encrypted RNA, or the LNP-formulated control mRNA encoding a GFP. The culture media was D10. Typically, cells were cultured in 24-well plates and were treated with about 15-150 ng of mRNA or about 15-150 ng of encrypted RNA per well (about 7.5-75 ng/cm²). Approximately 24 h later, cells were infected with a SARS-COV-2 isolate at a multiplicity of infection of 0.01 and returned to growth at 37° C. in a humidified 5% CO₂ atmosphere. Samples were collected at 48 hours post-infection and the level of infectious virus titrated by plaque assay on Vero E6, Vero-hACE2-TMPRSS2, or Huh7 cells as described for SARS-COV-2 in (Wong et al., Nature 2022; 605 (7908): 146-151, DOI: 10.1038/s41586-022-04630-3).

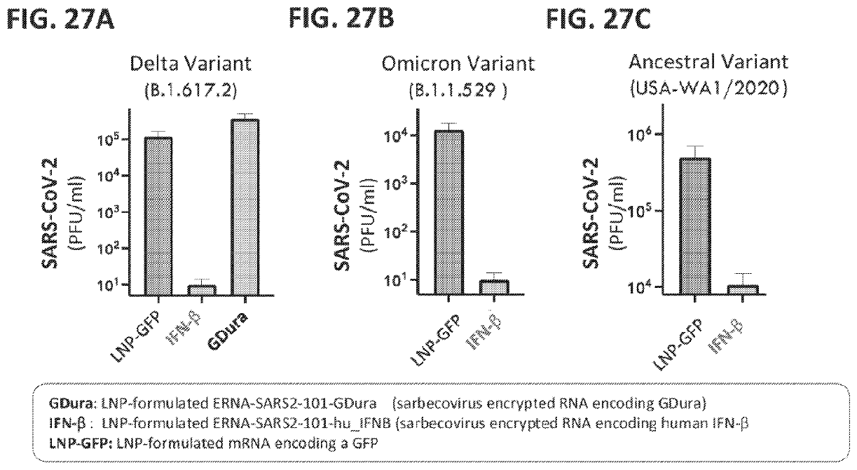

FIG. 27 shows that treatment of cells with an LNP-formulated therapeutic sarbecovirus encrypted RNA (ERNA-SARS2-101-hu_IFNB) substantially reduced SARS-COV-2 viral loads for a panel of diverse SARS-COV-2 variants, including: USA/WA1/2020 (“ancestral” or “Wuhan” variant), a Delta variant (Variant B.1.617.2), and an Omicron BA.1 (B.1.1.529) variant. For each tested SARS-CoV-2 variant, SARS-COV-2 viral loads were reduced by 10-10,000× in cell cultures treated with the LNP-formulated therapeutic sarbecovirus encrypted RNA (ERNA-SARS2-101-hu_IFNB) vs. the levels of SARS-COV-2 in matched cell cultures infected with the same SARS-COV-2 variant but treated with an LNP-formulated control RNA (either ERNA-SARS2-101-GDura or an mRNA encoding a GFP). The same LNP formulation was used to formulate ERNA-SARS2-101-hu_IFNB, ERNA-SARS2-101-GDura, and the mRNA encoding a GFP.

To further test the pan-sarbecovirus activity of a sarbecovirus encrypted RNA, Vero-hACE2-TMPRSS2 cells were treated an LNP-formulated ERNA-SARS2-001-GDura and infected with one of the following SARS-COV-2 variants at an MOI of 1 approximately 24 hours after treatment: USA/WA1/2020 (“Ancestral” or “WA1”), Beta (B.1.351), Delta (B.1.617.2), an Omicron BA.4 isolate, an Omicron BA.5 isolate, or “MA30”. MA30 denotes SARS2-N501YMA30, a mouse adapted strain of SARS-COV-2 described in (Wong et al., Nature 2022; 605 (7908): 146-151, DOI: 10.1038/s41586-022-04630-3).

FIG. 46 shows that the activation of a sarbecovirus encrypted RNA (ERNA-SARS2-001-GDura) was increased by ~2 logs or more in Vero-hACE2-TMPRSS2 cells treated with the encrypted RNA and infected by any of the tested SARS-COV-2 variants. The activation of the LNP-formulated sarbecovirus encrypted was measured by quantifying the levels of secreted luciferase in infected and uninfected cells, as described above.

As a control experiment in FIG. 46, the ability of a non-sarbecovirus (influenza A/PR8) to activate the LNP-formulated sarbecovirus encrypted RNA was also tested by quantifying the level of luciferase secretion in cells infected with influenza A/PR8 and subsequently treated with LNP-formulated ERNA-SARS2-001-GDura as in Example 21 (influenza A/PR8 at MOI 0.01), A549 cells. No substantial activation of the sarbecovirus encrypted RNA was measured in influenza infected cells. The sarbecovirus encrypted RNA was not substantially activated by influenza infection-because the influenza variant does not encode a translation activator for the sarbecovirus encrypted RNA.

### Example 22: Construction of RSV Encrypted RNAs

### RSV Antisense Encrypted RNA Scaffolds

DNA sequences were cloned by standard molecular biology methods as described in Example 1. For RSV antisense encrypted RNAs, some L and R flanking nucleotide sequences were obtained by concatenation of nucleotide regions from a publicly available human RSV strain A2 genome sequence (NCBI GenBank ID: KT992094.1). All nucleotide numbering is in reference to this nucleotide sequence. The L flank sequence (antis_5p_RSV, SEQ ID NO: 158) is comprised of a 227 nt region (nt 1-227) from the 5′ end of the RSV RNA (5′ trailer of L gene end sequence and NS1 gene start) with point mutations introduced around nt 142-146 (TATAT to CGTAC). The R flank (antis_3p_RSV, SEQ ID NO: 169) is comprised of the terminal 98 nt from the 3′ end of the RSV RNA comprising the untranslated region, gene start, and 3′-Leader sequences, with point mutations introduced upstream of the gene start and within 4 nucleotides of the 3′ end (CCGT to GCGT).

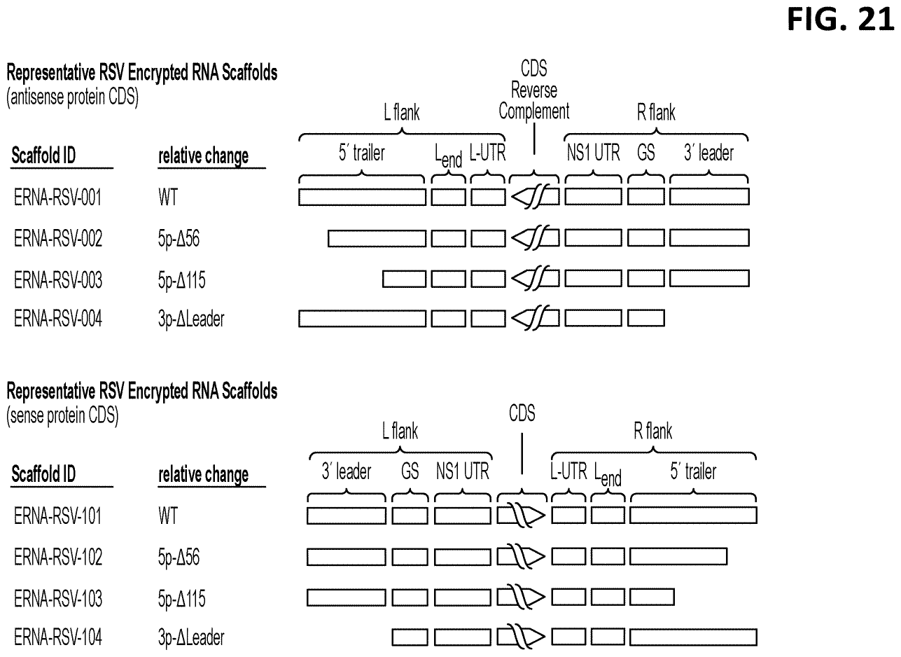

Two additional L flank sequences were developed which lacked either 56 nt (antis_5p_RSV_Δ56, SEQ ID NO: 168) or 115 nt (antis_5p_RSV_Δ115, SEQ ID NO: 167) of the 5′ end of RSV. Alternative R flank sequences were also developed: (i) antis_3p_RSV_Δleader (SEQ ID NO: 178) which lacked the terminal Leader sequence present in antis_3p_RSV (SEQ ID NO: 169); and (ii) antis_3p_RSV_36TrC (SEQ ID NO: 170) which alters the extreme 3′ end to enhance protein translation of the encoded protein. Some details of these sequence elements are depicted in a simplified schematic in FIG. 21.

Further L flank sequences were developed which modified the 5′ terminal nucleotides for compatibility with co-transcriptional capping systems: antis_5p_RSV_add_agg (SEQ ID NO: 163) and antis_5p_RSV_ag (SEQ ID NO: 164). A corresponding R flank sequences was additionally engineered: antis_3p_RSV_36TrC_ag (SEQ ID NO: 175).

The initial RSV antisense encrypted RNA scaffolds were obtained by combining three independent sequence blocks developed above. For example, ERNA-RSV-001-GDura was generated by concatenation of antis_5p_RSV (SEQ ID NO: 158), the reverse complement of the coding sequence of GDura (rcCDS_GDura, SEQ NO 301), and antis_3p_RSV (SEQ ID NO: 169) in 5′ to 3′ order. This construction strategy is depicted in a simplified schematic in FIG. 21. Two new RSV antisense encrypted RNAs incorporating the modified L flanks (5′ ends) were constructed similarly to ERNA-RSV-001-GDura. Both constructs retained the same R flank and antisense coding sequence as ERNA-RSV-002-GDura, but substituted a different 5′ sequence: ERNA-RSV-002-GDura used antis_5p_RSV_Δ56 (SEQ ID NO: 168) whereas ERNA-RSV-003-GDura used antis_5p_RSV_Δ115 (SEQ ID NO: 167). Similarly, ERNA-RSV-004-GDura was constructed by concatenation of antis_5p_RSV (SEQ ID NO: 158), rcCDS_GDura (SEQ ID NO: 301), and antis_3p_RSV_Δleader (SEQ ID NO: 178) in 5′ to 3′ order. ERNA-RSV-005-GDura was constructed analogously by concatenation of antis_5p_RSV (SEQ ID NO: 158), rcCDS_GDura (SEQ ID NO: 301), and antis_3p_RSV_36TrC (SEQ ID NO: 170) in 5′ to 3′ order. Some examples of additional RSV antisense encrypted RNA scaffolds are listed in Table 2.

### RSV Sense Encrypted RNA Scaffolds

RSV sense encrypted RNAs scaffolds were also prepared, which are in essence reverse complements of the above antisense scaffolds. To form sense RSV encrypted RNA scaffolds, an L flank (sense_5p_RSV, SEQ ID NO: 148) which is the reverse complement of antis_3p_RSV (SEQ ID NO: 169) was developed. Similarly, the R flank (sense_3p_RSV, SEQ ID NO: 154) is the reverse complement of antis_5p_RSV (SEQ ID NO: 158). An alternative L flank sequence was developed (sense_5p_RSV_Δleader, SEQ ID NO: 153), which is the reverse complement of antis_3p_RSV_Δleader (SEQ ID NO: 178). Two additional R flank sequences were developed: (i) sense_3p_RSV_Δ56 (SEQ ID NO: 157), which is the reverse complement of antis_5p_RSV_456 (SEQ ID NO: 168); and (ii) sense_3p_RSV_Δ115 (SEQ ID NO: 156), which is the reverse complement of antis_5p_RSV_Δ115 (SEQ ID NO: 167).

The initial sense RSV encrypted RNA scaffolds were obtained by combining three independent sequence blocks described above. For example, ERNA-RSV-101-GDura (the reverse complement of ERNA-RSV-001-GDura) was generated by concatenation of sense_5p_RSV (SEQ ID NO: 148), CDS_GDura (SEQ ID NO: 273), and sense_3p_RSV (SEQ ID NO: 154) in 5′ to 3′ order. This construction strategy is depicted in a simplified schematic in FIG. 21. Two alternative sense RSV scaffolds with modified R flank sequences were constructed similarly. Both constructs retained the same L flank and sense coding sequence as ERNA-RSV-101-GDura, but substituted a different 3′ R sequence: ERNA-RSV-102-GDura used sense_3p_RSV_Δ56 (SEQ ID NO: 157) whereas ERNA-RSV-103-GDura used sense_3p_RSV_Δ115 (SEQ ID NO: 156).

An additional alternative sense RSV encrypted RNAs incorporating a modified L flank was constructed similarly: ERNA-RSV-104-GDura was constructed by concatenation of sense_5p_RSV_Δleader (SEQ ID NO: 153), CDS_GDura (SEQ ID NO: 273), and sense_3p_RSV (SEQ ID NO: 154) in 5′ to 3′ order. Some examples of additional RSV sense encrypted RNA scaffolds are listed in Table 2.

### Development of Multigenic RSV Encrypted RNA Scaffolds

RSV antisense encrypted RNAs scaffolds were further developed that would encode more than one protein payloads. In the case of RSV antisense encrypted RNA scaffolds, a new sequence antis_I1_RSV_NS2 (SEQ ID NO: 415) was developed to permit transcription of separate mRNA payloads in the presence of a target-specific translation activator.

The initial RSV antisense encrypted RNA scaffolds with bi-genic payloads were obtained by combining five independent sequence blocks developed above. For example, ERNA-RSV-001-GDura-I1-IFNB was generated by concatenation of antis_5p_RSV (SEQ ID NO: 158), the reverse complement of the coding sequence of GDura (rcCDS_GDura, SEQ NO 301), antis_11_RSV_NS2 (SEQ ID NO: 415), the reverse complement of the coding sequence of IFNB (rcCDS_human_IFN_beta_1_precursor, SEQ ID NO: 306), and antis_3p_RSV (SEQ ID NO: 169) in 5′ to 3′ order. Using the same scaffold, but varying the order of the two coding sequences generated ERNA-RSV-001-IFNB-11-GDura by concatenation of antis_5p_RSV (SEQ ID NO: 158), the reverse complement of the coding sequence of IFNB (rcCDS_human_IFN_beta_1_precursor, SEQ ID NO: 306), antis_I1_RSV_NS2 (SEQ ID NO: 415), the reverse complement of the coding sequence of GDura (rcCDS_GDura, SEQ NO 301), and antis_3p_RSV (SEQ ID NO: 169) in 5′ to 3′ order.

Introduction of Additional Protein Payloads into RSV Encrypted RNA Scaffolds

The above 9 RSV encrypted RNA scaffolds shown with GDura as the polypeptide of interest were used as a basis to similarly encode alternative polypeptides of interest as listed in Table 5 or Table 10, including: EGFP, RLuc8, human IFN-β, human IFN-lambda1, human IFN-lambda3, mouse IFN-β, mouse IFN-lambda2, mouse IFN-lambda3, Syrian hamster IFN-β, or domestic ferret IFN-β. RSV antisense scaffolds encoding alternative polypeptides of interest were created by replacing rcCDS_GDura in ERNA-RSV-001-GDura, -002-GDura, -003-GDura, -004-GDura, or -005-GDura with an antisense CDS selected from Table 5 or Table 10. RSV sense encrypted RNA scaffolds encoding alternative polypeptides of interest were created by replacing CDS_GDura in ERNA-RSV-101, -102-GDura, -103-GDura, or -104-GDura with a sense CDS selected from Table 5 or Table 10. Following the convention of the previous nomenclature, “ERNA-RSV-X-Z” indicates that RSV encrypted RNA Scaffold X encodes polypeptide of interest Z.

Bigenic RSV antisense encrypted RNA scaffolds encoding for two proteins can be generated by replacing the rcCDS_GDura sequence (SEQ ID NO: 301) in ERNA-RSV-001-GDura-11-IFNB with an rcCDS selected from Table 5 or Table 10 and similarly replacing rcCDS_human_IFN_beta_1_precursor (SEQ ID NO: 306) in ERNA-RSV-001-GDura-I1-IFNB with an rcCDS selected from Table 5 or Table 10.

### DNA-Encoding of RSV Encrypted RNA for In Vitro Production of RNA

To produce capped RSV encrypted RNA or 5′-triphosphorylated RSV encrypted RNA, RSV encrypted scaffolds were cloned into the pAT201 vector described in Example 1.

An alternative in vitro transcription template to generate 5′-monophosphorylated RSV antisense encrypted RNA was created using the strategy described in Example 1. Plasmid templates in the pAT225-X family were constructed by concatenation of an alternative T7 promoter (Promoter_T7_AT, SEQ ID NO: 332), RSV antisense hammerhead ribozyme sequence (UTR_5p_RSV_antis_HHRz, SEQ ID NO: 7506), RSV antisense encrypted RNA, and convenient 3′ SapI restriction site (RE_SapI_3p, SEQ ID NO: 7201). For simplicity this construct is referred to as pAT225-X, where X is the RSV antisense encrypted RNA scaffold.

Plasmid family pAT226-X was created similarly to pAT225-X, except RSV antisense hammerhead ribozyme sequence for Delta56 (UTR_5p_RSV_antis_Delta56_HHRz, SEQ ID NO: 7507) was substituted for the original hammerhead ribozyme sequence. Likewise, in pAT227-X, RSV antisense hammerhead ribozyme sequence for Delta115 (UTR_5p_RSV_antis_Delta115_HHRz, SEQ ID NO: 7508) was substituted for the original hammerhead ribozyme sequence.

Plasmid family pAT228-X and plasmid family pAT229-X were created to facilitate the production of 5′-monophosphorylated RSV sense encrypted RNA. pAT228-X was created analogously to pAT225-X, except: (i) an RSV sense hammerhead ribozyme sequence (UTR_5p_RSV_sense_HHRz, SEQ ID NO: 7509) was substituted for the original hammerhead ribozyme sequence; (ii) an RSV sense encrypted RNA scaffold was substituted for an RSV antisense encrypted RNA scaffold. Likewise, pAT229-X was created analogously to pAT225-X, except: (i) RSV sense hammerhead ribozyme sequence for DeltaLeader (UTR_5p_RSV_sense_DeltaLeader_HHRz, SEQ ID NO: 7510) was substituted for the original hammerhead ribozyme sequence; (ii) an RSV sense encrypted RNA scaffold was substituted for an RSV antisense encrypted RNA scaffold.

### Example 23: Activation of RSV Encrypted RNA by RSV Infection or by a Translation Activator Comprising an RSV RdRP

At 24 hours before infection, HEK-293T cells were plated at a concentration of 400,000 cells/well in a 24-well dish. At 0 hpi, culture media was removed and cells were infected with the RSV A2 strain virus by adding the virus inoculum into a total volume of 250 μL Opti-MEM per well. The cells were infected at one of MOIs: 10, 1, or 0. Following incubation at 37° C. with 5% CO₂ for 2 h, the virus inoculum was removed and 250 μL of D02 media was added to cells.

Encrypted RNAs were formulated into LMAX-LNPs as described above in Example 8. LMAX-LNP-formulated encrypted RNAs were added dropwise to cells in D02 media and the cultures allowed to recover in humidified 5% CO₂ incubators at 37° C.

At 24, 48, or 72 hpi, GDura protein production from an RSV encrypted RNA encoding GDura as the polypeptide of interest was estimated by collecting culture supernatants and analyzing the production of secreted luciferase in the presence or absence of RSV infection. Per the above, 10 μL of the tissue culture supernatant was added to 90 μL of luciferase coelenterazine substrate and analyzed in a Victor 3 luminometer (Perkin-Elmer). Low levels of background translation of luciferase (the polypeptide of interest) encoded in RSV encrypted RNA was observed in the absence of RSV infection.

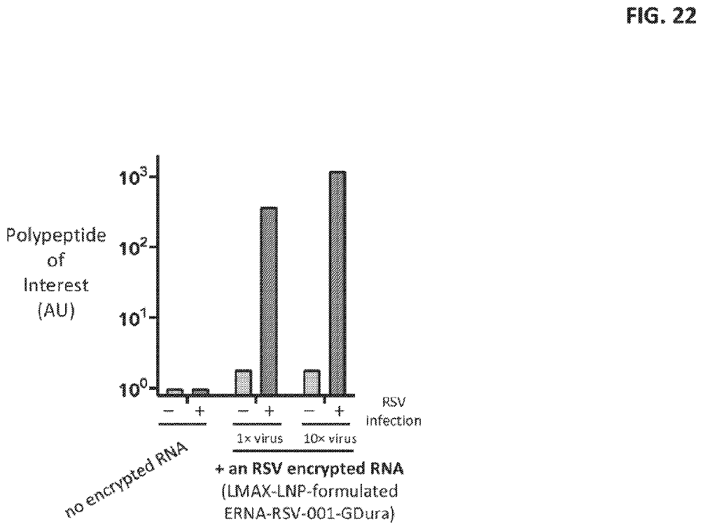

FIG. 22 demonstrates that an RSV encrypted RNA (ERNA-RSV-001-GDura) is activated when cells treated with the encrypted RNA are infected with RSV A2 at an MOI of 1 (1× in FIG. 22) or 10 (10× in FIG. 22). Translated levels of the GDura polypeptide of interest increased approximately 2-3 log (100-1000×) in the presence of RSV infection. Only low levels of GDura were observed when treatment with the encrypted RNA was omitted or when cells were not infected with RSV.

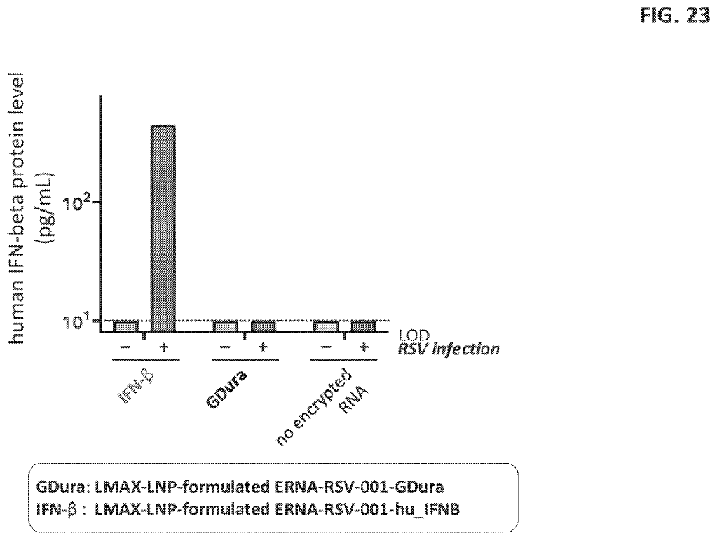

FIG. 23 shows that a therapeutic RSV encrypted RNA (i.e. an RSV SHIELD) can be activated by infection with RSV A2 to produce increased levels of a therapeutic protein (human IFN-β). Human 293T cells were treated with an LMAX-LNP-formulated encrypted RNA encoding GDura (ERNA-RSV-001-GDura) or an LMAX-LNP-formulated therapeutic encrypted RNA encoding human IFN-β (ERNA-RSV-001-hu_IFNB) or were not treated with an encrypted RNA. Cultures were infected with RSV A2 or not infected. At 72 hours after infection (about 70 h after treatment with encrypted RNAs or controls), the level of secreted human IFN-β in the culture media was quantified by ELISA.

In this experiment, substantially increased human IFN-β production (450 pg/mL of cell culture supernatant) was only observed in RSV SHIELD-treated cells (ERNA-RSV-001-hu_IFNB) and only in cells infected with RSV. In contrast, low levels of human IFN-β (assigned to the limit of detection at 10 μg/mL) were produced without RSV infection or when cells are treated with a control encrypted RNA (ERNA-RSV-001-GDura). The absence of substantial observed immunogenicity (here, secretion of human IFN-β) from a non-therapeutic encrypted RNA shows that even though some encrypted RNAs incorporate viral regulatory elements, encrypted RNAs can be substantially non-immunogenic if they do not encode immunogenic polypeptides of interest. The absence of substantial observed immunogenicity of ERNA-RSV-001-hu_IFNB in the absence of viral infection shows that, even if encrypted RNAs encode immunogenic polypeptides of interest, encrypted RNAs can remain substantially non-immunogenic until they undergo activation by a translation activator.

Translation Activation of RSV Encrypted RNAs by RSV Infection does not Require 5′ Phosphorylation of Encrypted RNAs

The effect of 5′ phosphorylation state (monophosphate, triphosphate, or no phosphate) of RSV antisense encrypted RNAs was investigated next.

5′-triphosphorylated RSV antisense encrypted RNAs were prepared via IVT from a plasmid template vector (pAT201) using a standard equimolar mixture of ATP, CTP, GTP, and UTP as described above in Example 6.

5′-monophosphorylated RSV antisense encrypted RNAs were prepared via IVT from a plasmid template vector (pAT225, pAT226, or pAT227) which added a hammerhead ribozyme sequence to the 5′ end of RNA transcript. Post-transcription, self-cleavage of the hammerhead ribozyme divides the full length IVT product into a shorter 5′-triphosphorylated sequence and a much longer 5′-monophosphorylated encrypted RNA. Purification of the larger encrypted RNA transcript via HPLC (see Example 7) allows for the production of uncapped, 5′-monophosphorylated RNA.

5′-nonphosphorylated RNAs was prepared by enzymatic treatment of RNA with recombinant shrimp alkaline phosphatase (“rSAP”, New England Biolabs, cat. No. M0371S) per the manufacturer's instructions.

5′-triphosphorylated and 5′-monophosphorylated RSV antisense encrypted RNAs were prepared as described above using the ERNA-RSV-001-GDura or ERNA-RSV-001-hu_IFNB scaffolds, then formulated into LMAX-LNPs as described in Example 8.

At 24 hours before infection, HEK-293T cells were plated at a concentration of 400,000 cells/well in a 24-well dish. At 0 hpi, culture media was removed and cells were infected with RSV A2 strain virus by adding the virus inoculum in a total volume of 250 μL Opti-MEM per well. The cells were infected at one of MOIs: 1, 0.1, or 0. Following incubation at 37° C. with 5% CO₂ for 2 h, the virus inoculum was removed and 250 μL of D02 media was added to cells.

At about 2 hpi, cells were treated with LMAX-LNP-formulated encrypted RNA or control substances by dropwise addition to cells in the D02 media and the cultures allowed to recover in humidified 5% CO₂ incubators at 37° C. Cultures were treated with about 250 ng of LMAX-LNP-formulated encrypted RNA.

At 24, 48, or 72 hpi, luciferase protein production from RSV encrypted RNAs was estimated by collecting culture supernatants and analyzing the production of secreted luciferase in the presence or absence of RSV infection. Per the above methods, 10 μL of the tissue culture supernatant was added to 90 μL of luciferase coelenterazine substrate and analyzed in a Victor 3 luminometer (Perkin-Elmer).

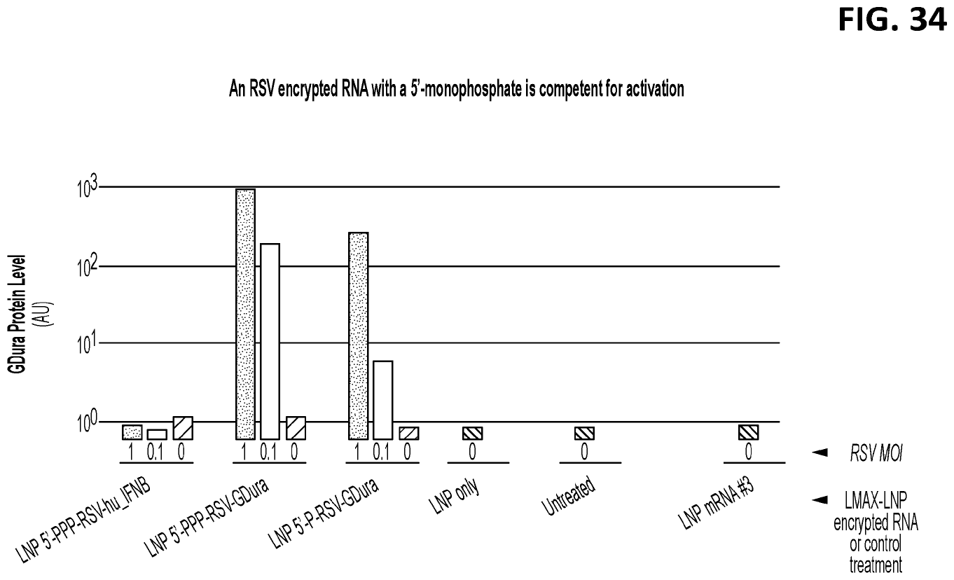

FIG. 34 shows that cells which received an LMAX-LNP-formulated 5′-triphosphorylated or 5′-monophosphorylated ERNA-RSV-001-GDura produced high levels of the GDura polypeptide of interest when infected with RSV (MOI of 1), with GDura levels increasing approximately 100-1000× due to RSV infection. Thus, RSV antisense encrypted RNAs can be activated whether the encrypted RNAs have 5′-ends with a monophosphate or triphosphate moiety. Treatment of cells with 5′-triphosphorylated ERNA-RSV-001-hu_IFNB or with non-encrypted mRNA controls (encoding a non-luciferase protein) did not yield substantial GDura production. In all cases, approximately background levels of luciferase activity were observed from the RSV encrypted RNA encoding GDura in the absence of RSV infection.

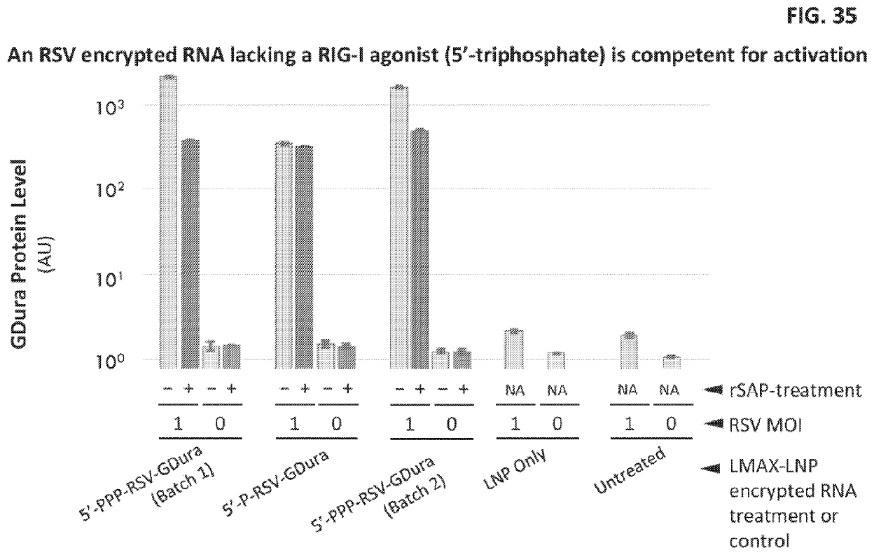

Results from a similar experiment are shown in FIG. 35, which included RSV encrypted RNAs incorporating 5′-nonphosphorylated RNAs (via rSAP treatment) and limited infection to an MOI of 1 or 0.

FIG. 35 shows that cells treated with an LMAX-LNP-formulated 5′-triphosphorylated, 5′-monophosphorylated, or 5′-nonphosphorylated ERNA-RSV-001-GDura produced high levels of the GDura when infected with RSV, with GDura levels increasing approximately 100-1000× in the presence of RSV infection. In this experiment, two production batches of 5′-triphosphorylated ERNA-RSV-001-GDura were tested (labelled Batch 1 and Batch 2, respectively); the two batches produced similar results. This extends the results of FIG. 34 and shows that RSV antisense encrypted RNAs can be activated by translation activators when the uncapped encrypted RNAs have 5′-ends with 0, 1, or 3 phosphates. Treatment of cells with 5′-triphosphorylated non-encrypted RNA (e.g. an mRNA encoding a non-luciferase protein) or other controls (e.g. LMAX-LNP without RNA) did not yield substantial GDura production. In all cases, approximately background levels of luciferase activity from the RSV encrypted RNA encoding GDura were observed in the absence of RSV infection.

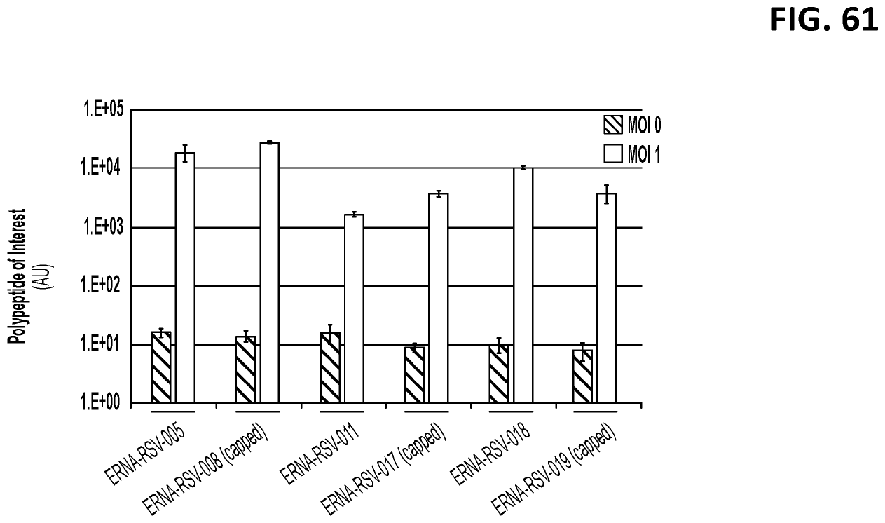

FIG. 61 shows that an antisense RSV encrypted RNA can be modified with a 5′-cap without losing the ability to be substantially activated by RSV infection. Notably, capping of the RSV encrypted RNA utilized an additional three-nucleotide AGG sequence added at the 5′-end of the L-region of the encrypted RNA to enable co-transcriptional capping via CleanCap AG (TriLink, cat. No. N-7113). In this example, 5′-capped and uncapped variants of the same encrypted RNA (e.g. ERNA-RSV-008-GDura) demonstrated similar levels of activation in response to RSV infection, as measured by the luciferase assay in treated cells.

### Nucleoside-Modified RSV Encrypted RNAs can be Activated by RSV Infection

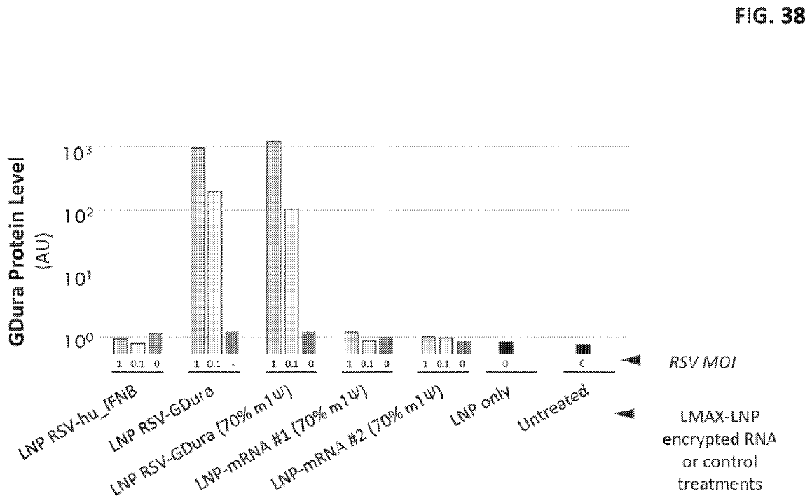

FIG. 38 shows that RSV antisense encrypted RNAs incorporating modified nucleotides can retain their ability to be activated by RSV infection. Encrypted RNAs were prepared using the ERNA-RSV-001 scaffold (and encoded either GDura or human IFN-β as a polypeptide of interest).

Encrypted RNAs were prepared in vitro transcription as described above, (but left uncapped), as described above in Example 6. Two different nucleotide pools were used: (i) “100% U”, an equimolar solution of ATP, CTP, GTP, and UTP during in vitro transcription; (ii) “70% m14”, analogous to the previous but the ‘UTP’ pool was comprised of a binary mixture of 30% UTP and 70% N1-methylpseudouridine triphosphate (percentages are % by mol). RSV antisense encrypted RNAs were then formulated into LMAX-LNPs as described in Example 8.

At 24 hours before infection, 239T cells were plated at a concentration of 250,000 cells/well in 24-well microplates. At ~0 hpi, parallel cultures were infected with RSV A2 at an MOI of 0, 0.1, or 1. To perform the infection, culture media was removed and cells were infected by adding the virus inoculum in a total volume of 250 μL Opti-MEM per well. Following incubation at 37° C. with 5% CO₂ for 2 h, the virus inoculum was removed and 250 μL of D02 media was added to cells.

At about 2 hpi, cells were treated with LMAX-LNP-formulated encrypted RNA or control substances by dropwise addition to cells to the D02 media and the cultures allowed to recover in humidified 5% CO₂ incubators at 37° C. Cultures were treated with about 250 ng of LNP-formulated encrypted RNA.

At 72 hpi, GDura protein expression from RSV encrypted RNA in the presence or absence of RSV infection was measured by collecting culture supernatants and analyzing the production of secreted luciferase due to infection by RSV.

As shown in FIG. 38, nucleoside-modified (70% m1Ψ) or non-nucleoside modified RSV encrypted RNAs can be activated in cells infected with RSV. FIG. 38 shows that cells treated with LMAX-LNP-formulated nucleoside-modified (70% m1Ψ) or non-nucleoside modified ERNA-RSV-001-GDura produced comparably high levels of the GDura polypeptide of interest when infected with RSV, with GDura levels increasing ~1000× in the presence of RSV infection with MOI of 1. Thus, RSV antisense encrypted RNAs can be activated even when the encrypted RNAs incorporate modified nucleotides (i.e., are nucleoside-modified). Treatment of cells with ERNA-RSV-001-hu_IFNB or with non-encrypted mRNA controls (encoding a non-luciferase protein) did not yield substantial GDura production. Whether nucleoside-modified or unmodified, approximately background levels of luciferase activity were observed from the RSV encrypted RNAs encoding GDura in the absence of RSV infection.

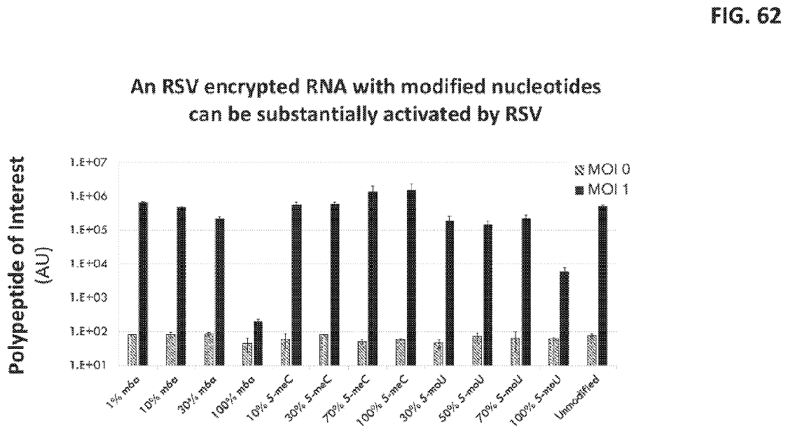

FIG. 62 shows that, in some embodiments, an RSV antisense encrypted RNA can incorporate up to 100% modified nucleotides without losing the ability to be substantially activated by RSV infection. A capped RSV encrypted RNA (ERNA-RSV-008-GDura) was shown to be able to incorporate at least up to 30% N6-methyladenosine (“m6a”), up to 100% 5-methylcytidine (“5-meC”), or up to 70% 5-methoxyuridine (“5-moU”) without substantially reduced activation (measured at 72 h post infection in HEp-2 cells) in response to RSV infection. Unmodified in this drawing shows the encrypted RNA with 100% uridine.

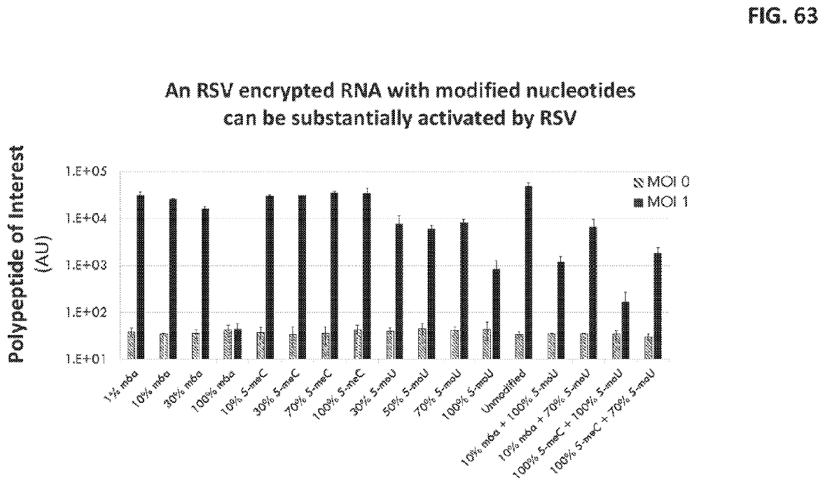

FIG. 63 further demonstrates that the RSV encrypted RNA incorporating a combination of two modified nucleotides such as 10% N6-meA and 70% 5-meOU can be substantially activated by RSV infection (here measured at 48 h post RSV infection in HEp-2 cells via the luciferase assay).

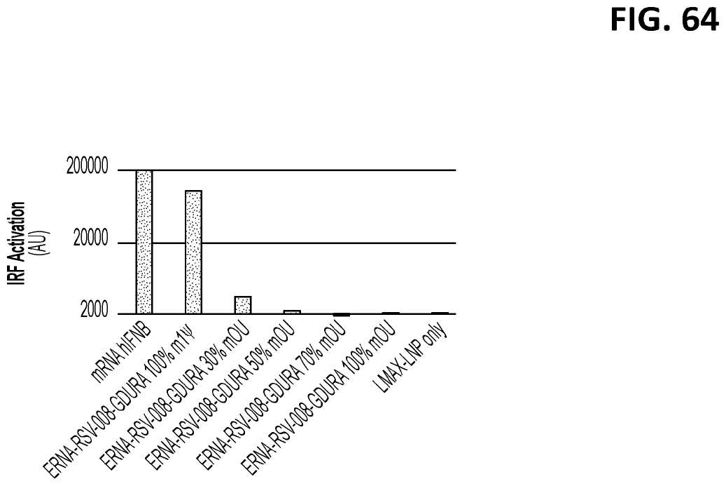

FIG. 64 shows that, in some embodiments, a nucleoside-modified RSV antisense encrypted RNA (ERNA-RSV-008-GDura) is significantly less immunogenic than an unmodified RSV encrypted RNA when provided to cells without a translation activator as measured by an interferon-stimulated gene reporter, IRF, in A549 Dual cells using the immunogenicity assay described in Example 19 above (i.e., production of luciferase in the A549 Dual cells is tied to an ISRE promoter, so that higher levels of immunogenicity result in higher levels of secreted luciferase, denoted IRF here).

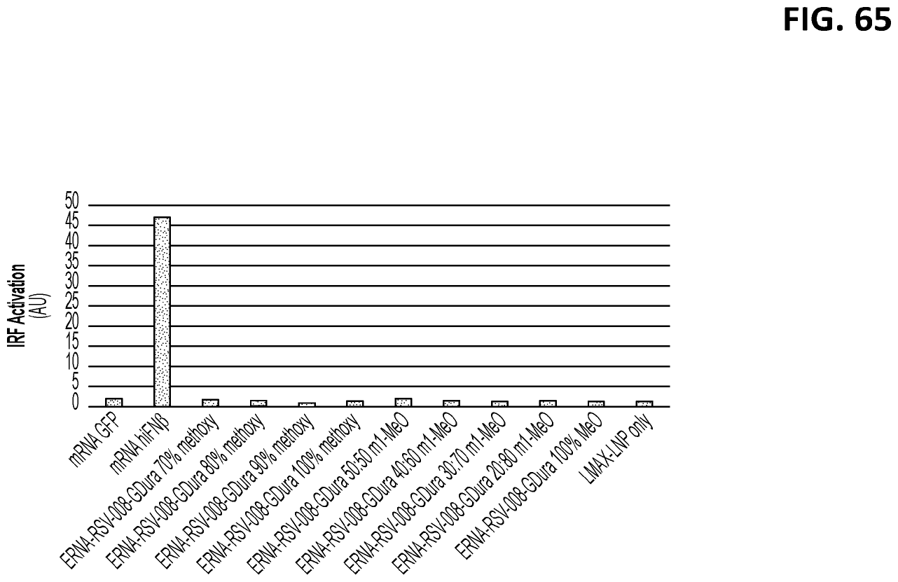

FIG. 65 shows additional embodiments wherein a nucleoside-modified RSV antisense encrypted RNA (ERNA-RSV-008-GDura) is significantly less immunogenic than nonmodified RSV encrypted RNA when provided to cells without the translation activator (as measured by an interferon-stimulated gene reporter, IRF, in A549 Dual cells following the approach in Example 19). Nucleoside-modifications shown include replacement of uridine with 5-methoxyuridine (“methoxy” or “MeO”), or a complete replacement of uridine with a binary mixture of N1-methylpseudouridine (“m1”) and 5-methoxyuridine (“MeO”). The ratio of the binary mixture is indicated by numerals separated by a colon—e.g., 30% N1-methylpseudouridine and 70% 5-methoxyuridine is indicated by “30:70 m1-meO”.

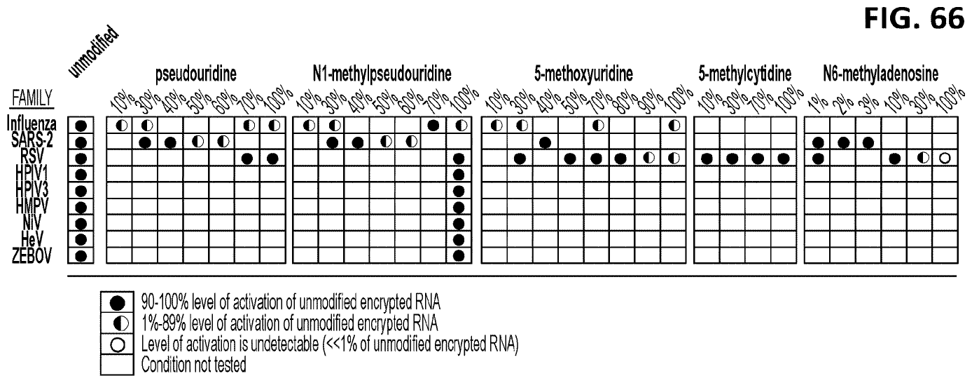

FIG. 66 shows a summary figure of some nucleoside-modified encrypted RNAs that can be activated in the presence of a translation activator. Some encrypted RNAs tested for compatibility with nucleoside-modification were: an influenza encrypted RNA (ERNA-IAV-002-GDura), a sarbecovirus (“SARS-2”) encrypted RNA (ERNA-SARS2-101-GDura), an RSV encrypted RNA (ERNA-RSV-008-GDura), an HPIV1 encrypted RNA (ERNA-HPIV1-002-GDura), an HPIV3 encrypted RNA (ERNA-HPIV1-003-GDura), an HMPV encrypted RNA (ERNA-HMPV-003-GDura), a henipavirus (“NiV”) encrypted RNA (ERNA-NiV-001-GDura), a henipavirus (“HeV”) encrypted RNA (ERNA-HeV-001-GDura), or a filovirus (“ZEBOV”) encrypted RNA (ERNA-ZEBOV-001-GDura).

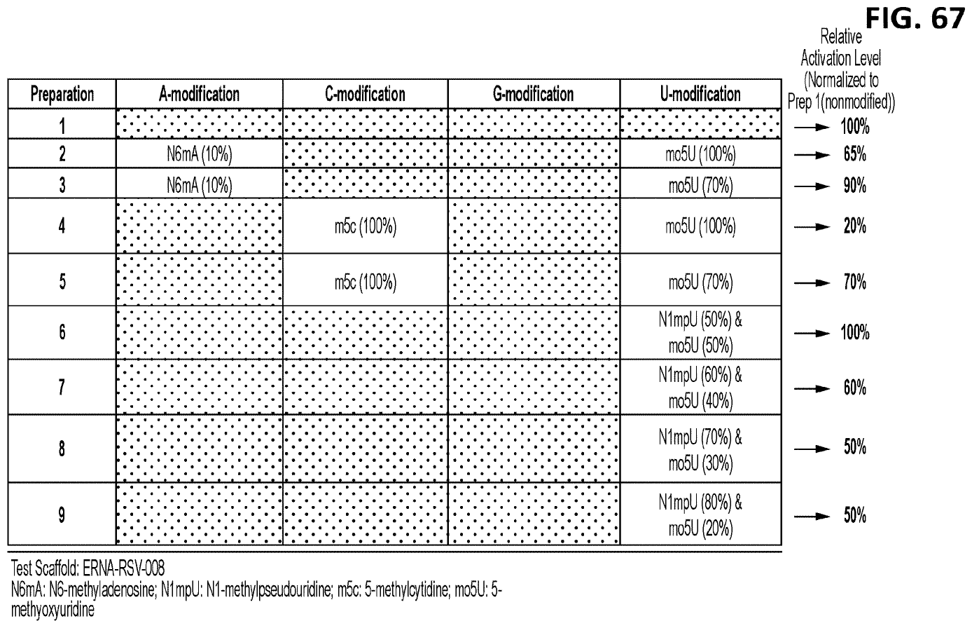

FIG. 67 shows that, in some embodiments, encrypted RNAs can be nucleoside-modified with more than one class of nucleoside and continue to retain activation by a translation activator. An RSV encrypted RNA (ERNA-008-GDura) was nucleoside-modified by A-modification (e.g. 10% N6-methyladenosine), C-modification (e.g., 100% 5-methylcytidine), U-modification (N1-methylpseudouridine or 5-methyoxyuridine or both) or by more than one class of modification. Activation values are reported as a percentage of the activation of the nonmodified encrypted RNA.

### RSV Antisense or RSV Sense Encrypted RNAs can be Activated by RSV Infection

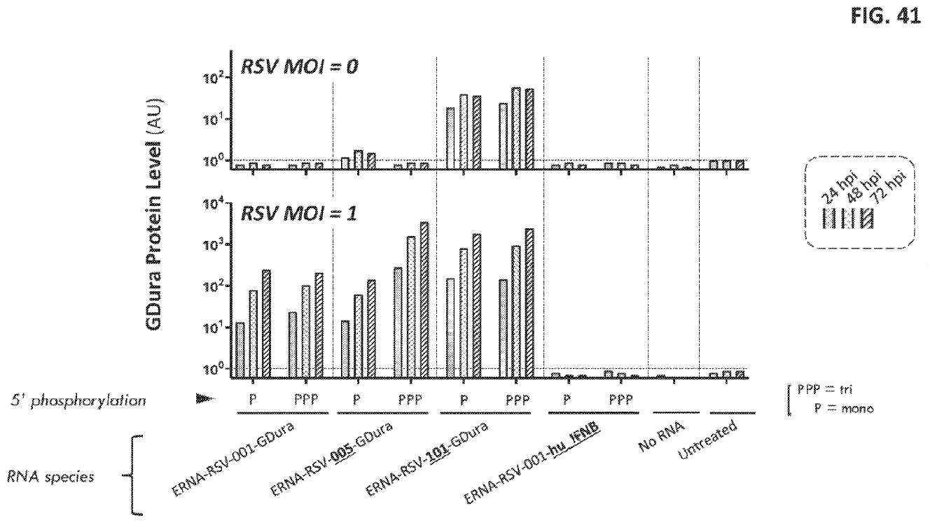

FIG. 41 shows that RSV antisense encrypted RNAs or RSV sense encrypted RNAs can be activated via RSV infection. Antisense encrypted RNAs were prepared using the ERNA-RSV-001-GDura scaffold or the ERNA-RSV-005-GDura scaffold (which contained a different R flanking sequence to enhance protein translation upon activation). Additional RSV antisense encrypted RNAs were prepared using ERNA-RSV-001-hu_IFNB or ERNA-RSV-001-m_IFNB scaffolds. Sense encrypted RNAs were prepared using the ERNA-RSV-101-GDura scaffold.

5′-triphosphorylated and 5′-monophosphorylated RSV antisense encrypted RNAs were prepared and purified as described above, then formulated into LMAX-LNPs as described in Example 8. Similarly, 5′-triphosphorylated and 5′-monophosphorylated RSV sense encrypted RNAs were prepared and purified as described above, then formulated into LMAX-LNPs as described in Example 8.

At 24 hours before infection, 239T cells were plated at a concentration of 250,000 cells/well in 24-well microplates At ~0 hpi, parallel cultures were infected with RSV A2 at an MOI of 0 or 1. To perform the infection, culture media was removed and cells were infected by adding the virus inoculum in a total volume of 250 μL Opti-MEM per well. Following incubation at 37° C. with 5% CO₂ for 2 h, the virus inoculum was removed and 250 μL of D02 media was added to cells.

At about 2 hpi, cells were treated with LMAX-LNP-formulated encrypted RNA or control substances by dropwise addition to cells in the D02 media and the cultures allowed to recover in humidified 5% CO₂ incubators at 37° C. Cultures were treated with about 250 ng of LNP-formulated encrypted RNA.

At 24, 48, or 72 hpi, GDura protein expression from RSV encrypted RNAs in the presence or absence of RSV infection was measured by collecting culture supernatants and analyzing the production of secreted luciferase due to infection by RSV.

FIG. 41 shows RSV sense encrypted RNAs and RSV antisense encrypted RNAs encoding GDura as the polypeptide of interest that can be activated by RSV infection. These sense and antisense encrypted RNAs can be activated by RSV whether the encrypted RNAs are 5′-triphosphorylated or 5′-monophosphorylated. In some cases, the RSV sense encrypted RNAs show higher background levels of GDura protein translation (in the absence of RSV infection) than the RSV antisense encrypted RNAs. In addition, an alternative RSV antisense encrypted RNA (ERNA-RSV-005-GDura) had substantially improved translation activation in the context of RSV infection when the encrypted RNA was 5′-triphosphorylated, producing GDura protein levels ~10-100× higher than the levels observed in parallel cell cultures infected with RSV and treated with 5′-triphosphorylated ERNA-RSV-001-GDura. A control encrypted RNA that did not encode a luciferase as the polypeptide of interest had approximately background levels of luciferase activity in uninfected and RSV-infected cells.

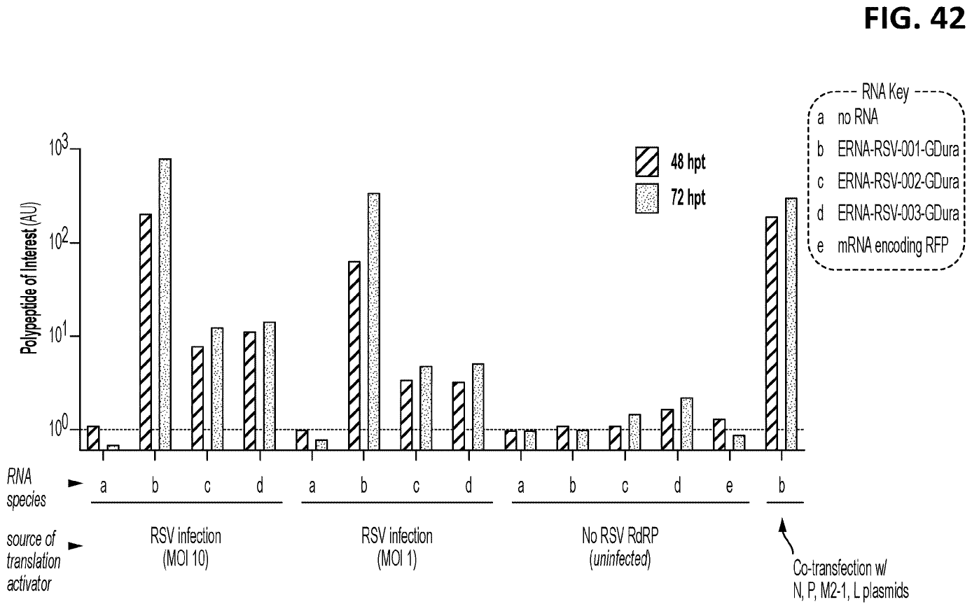

FIG. 42 shows an additional experiment using a similar methodology as FIG. 41 to: (i) quantify the activation of alternative RSV antisense encrypted RNAs; (ii) demonstrate that a translation activator for some RSV encrypted RNAs is comprised of four RSV proteins (N, P, M2-1, L). Using the same methods as FIG. 41, LMAX-LNP-formulated 5′-triphosphorylated RSV antisense encrypted RNAs were prepared from encrypted RNA scaffolds ERNA-RSV-001-GDura, ERNA-RSV-002-GDura, or ERNA-RSV-003-GDura. An LMAX-LNP-formulated capped mRNA encoding an RFP was used as a control.

The following were tested for the ability to function as translation activators: infection with RSV A2 at an MOI of 10; infection with RSV A2 at an MOI of 1; co-transfection of four expression plasmids using Lipofectamine 3000 (Thermo Fisher Scientific; cat. No. L3000015) with each expression plasmid producing a different RSV protein (N, P, M2-1, L), such that N, P, M2-1, and L are simultaneously produced within a given cell.

FIG. 42 shows that RSV encrypted RNAs can be activated by infection with RSV or by a translation activator provided in the absence of viral infection. Cells were treated at 2 hpi with one of three encrypted RNAs (ERNA-RSV-001-GDura, ERNA-RSV-002-GDura, or ERNA-RSV-003-GDura) that were formulated into LMAX-LNPs (~250 ng of LNP-formulated RNA per culture), following the method above. For each of the three RSV encrypted RNAs tested, the level of the GDura polypeptide of interest in RSV-infected cells at 72 hours post-infection (MOI of 10) increased approximately 1-3 log (10-1000×) above the level of GDura in cells analogously treated with the same LMAX-LNP-formulated encrypted RNA but not infected with RSV. Furthermore, providing a set of four plasmids encoding the RSV proteins (N, P, M2-1, L) to cells treated with LMAX-LNP-formulated ERNA-RSV-001-GDura, increased translation of the polypeptide of interest (GDura) by approximately 2-3 log (100-1000×) above the level of GDura in cells analogously treated with LMAX-LNP-formulated ERNA-RSV-001-GDura but not provided with the set of four RSV plasmids (encoding N, P, M2-1, L). Thus, a translation activator for RSV encrypted RNAs can be provided in the absence of viral infection (e.g. by co-transfecting cells with plasmids encoding a viral polymerase complex).

### Some RSV Encrypted RNAs can be Transmitted to New Cells Upon RSV Infection

As translation activators of some encrypted RNAs are comprised of proteins provided by viruses during infection, it is reasonable that some encrypted RNAs could have sufficient similarity to viral genomes such that they would be replicated via viral infection and potentially even encapsidated within viral particles and transmitted to new cells.

An experiment was performed to test the capacity of some RSV encrypted RNAs to be transmitted to untreated cells via: (i) infection with RSV; or (ii) by providing RSV N, P, M2-1, L proteins via transfection of expression plasmids.

An RSV antisense encrypted RNA was prepared as 5′-triphosphorylated ssRNA via IVT from an ERNA-RSV-001-GDura scaffold template. An mRNA encoding a red fluorescent protein (RFP) was used as a control.

At 24 hours before infection, 239T cells were plated at a concentration of 250,000 cells/well in 24-well microplates.

At ~0 hpi, parallel cultures were infected with RSV A2 at an MOI of 0, 1, or 10. To perform the infection, culture media was removed and cells were infected by adding the virus inoculum in a total volume of 250 μL Opti-MEM per well. Following incubation at 37° C. with 5% CO₂ for 2 h, the virus inoculum was removed and 250 μL of D02 media was added to cells. At about 6 hours after infection, cells were treated with LMAX-LNP-formulated encrypted RNA or control substances by dropwise addition to cells to the D02 media and the cultures allowed to recover in humidified 5% CO₂ incubators at 37° C. Cultures were treated with about 250 ng of LMAX-LNP-formulated encrypted RNA.

Parallel cultures were not infected with RSV, but were instead transfected with 4 expression plasmids each producing a distinct RSV protein (N, P, M2-1, or L), such that N, P, M2-1, and L are produced within a given cell. Transfection of plasmid DNA was accomplished using Lipofectamine 3000 (Thermo Fisher Scientific; cat. no. L3000015). These cells were simultaneously treated with LMAX-LNP-formulated encrypted RNA or control substances by dropwise addition to cells to the D02 media and the cultures allowed to recover in humidified 5% CO₂ incubators at 37° C. Cultures were treated with about 250 ng of LMAX-LNP-formulated encrypted RNA.

At 3 days after treatment of donor 293T cultures with encrypted RNA or control RNA, 100 μL of donor culture supernatant was used to inoculate 24-well plates of HEp-2 cells (250,000 cells/well). After 2 h of incubation, inocula were removed and the recipient cell monolayers washed extensively with DPBS (to remove any residual GDura protein carried over from the donor media), and fresh D02 media added to the recipient cells. At 0, 1, 2, or 3 days post-transfer, a portion of the culture media was removed and the level of GDura protein quantified as described above.

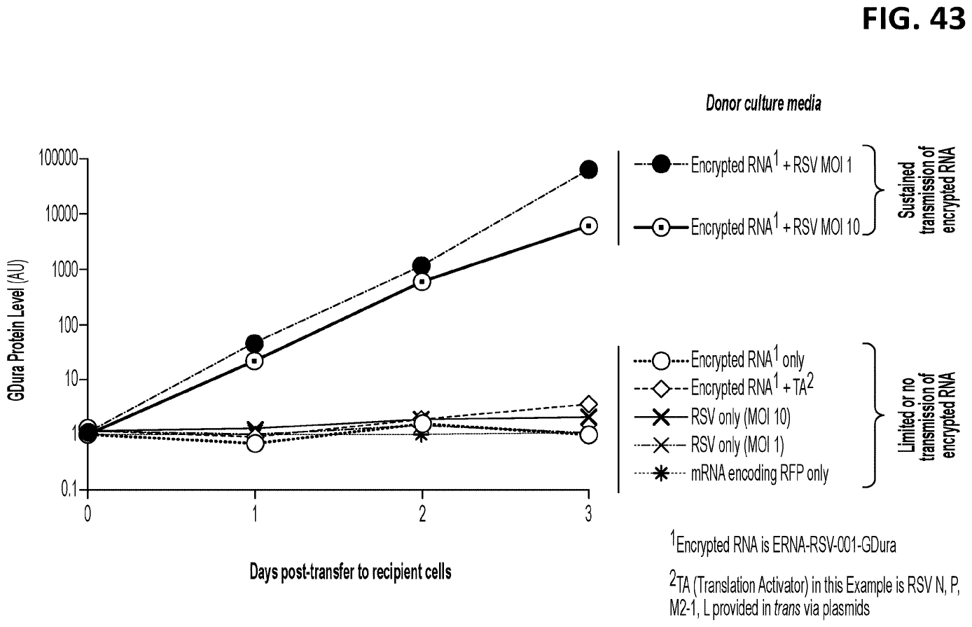

FIG. 43 shows that ERNA-RSV-001-GDura could be transmitted to new cells when donor 293T cells were treated with ERNA-RSV-001-GDura and infected with RSV (MOI 1 or 10) (indicated by the two upper curves in FIG. 43). The recipient HEp-2 cultures from these two donors showed an approximately exponential increase in GDura protein levels over the 3 days after the transfer, increasing up to 4 log. Since only the donor cells were infected with RSV, this indicated that both the encrypted RNA and the translation activator (i.e. the translation activator provided during RSV infection) were transmitted to the recipient cells from these two donor cultures and that the transmitted encrypted RNA was activated in the recipient cells.

In contrast, recipient cultures that received inocula from all other donor cultures (lower 5 flat curves in FIG. 43) did not show an increase in GDura protein levels over 3 days post-transfer, indicating that the encrypted RNA or the translation activator was not transmitted to new cells. In particular, treatment of the donor cells with expression plasmids encoding the 4 RSV proteins N, P, M2-1, L (shown in FIG. 42 to be a translation activator of ERNA-RSV-001-GDura) does not result in activation of GDura in the recipient cells. In this example, sustained transmission of an encrypted RNA to new cells requires viral infection.

### Example 24: RSV Encrypted RNAs Activated Using Different Strains of RSV

5′-triphosphorylated or 5′-monophosphorylated RSV encrypted RNAs were prepared as described in Example 23 (e.g. using the ERNA-RSV-001-GDura scaffold cloned into a suitable production plasmid). The encrypted RNAs were then formulated into LNPs as described in Example 9.

At 24 hours before infection, HEp-2 cells were plated at a concentration of 250,000 cells/well in 24-well microplates. At 0 hpi, parallel cultures were infected with RSV A2 or RSV B1 at an MOI of 1. More specifically, the infection was performed by removing the culture media and infecting cells with: (i) RSV A2 virus at an MOI of 0 or 1; or (ii) RSV B1 at an MOI or 0.1 or 1. The cultures were infected by adding the virus inoculum in a total volume of 250 μL Opti-MEM per well. Following incubation at 37° C. with 5% CO₂ for 2 h, the virus inoculum was removed and 250 μL of D02 was added to cells.

At 2 hpi, the cells were treated with LNP-formulated encrypted RNAs encoding GDura as the polypeptide of interest or control substances by dropwise addition of the articles to cells in D02 media and the cultures were allowed to recover in humidified 5% CO₂ incubators at 37° C. A representative dose was 100 ng or 200 ng of LNP-formulated encrypted RNA.

At 24, 48, or 72 hpi, the level of GDura translation was quantified in cultures treated with RSV encrypted RNA or controls in the presence or absence of RSV infection. A portion of the culture supernatants (e.g., 50 μL) was collected and used to quantify the production of secreted luciferase due to infection by RSV.

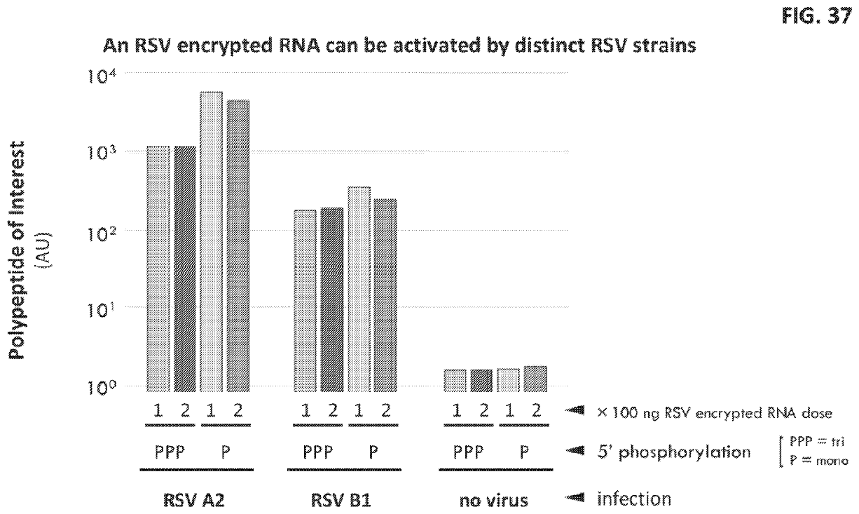

FIG. 37 shows that an LNP-formulated RSV encrypted RNA can be activated by diverse RSV variants whether the encrypted RNA is 5′-monophosphorylated or 5′-triphosphorylated. Both 5′-monophosphorylated and 5′-triphosphorylated variants of ERNA-RSV-001-GDura were activated when treated HEp-2 cells were infected by diverse RSV variants: RSV A2 or RSV B1. In fact, expression of the GDura polypeptide of interest was increased by >10× in the presence of RSV A2 or RSV B1 infection (compared to the level of GDura in analogously treated cells not infected with an RSV strain). This further indicates that activation of an encrypted RNA can occur after infection of treated cells with a variety of viral strains within a viral species, whether the encrypted RNA is 5′-monophosphorylated or 5′-triphosphorylated.

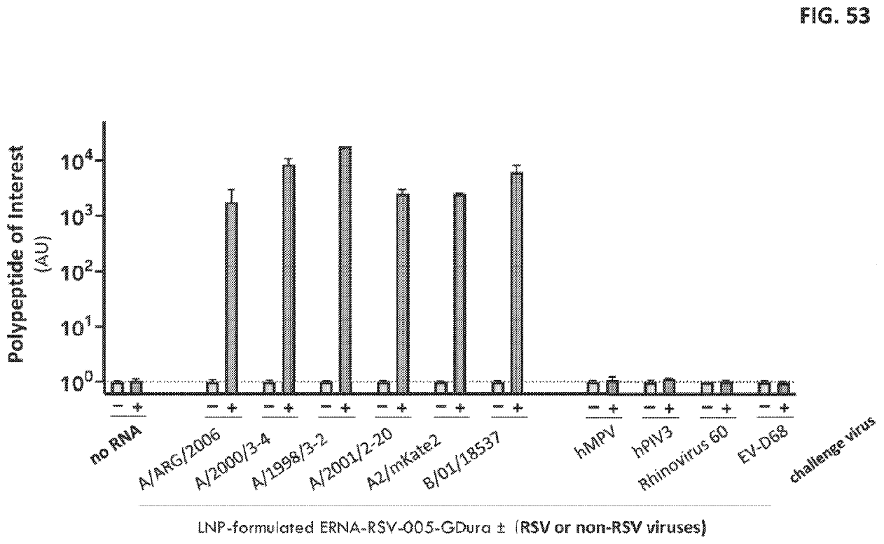

FIG. 53 shows that an LNP-formulated RSV encrypted RNA (ERNA-RSV-005-GDura) can be substantially activated by a diverse set of RSV A & B variants, including clinical isolates. HEp-2 cells treated with ERNA-RSV-005-GDura demonstrated substantial activation of the GDura polypeptide of interest (as measured by the luciferase assay at 72 hpi) when treated cells were infected by a panel of RSV variants, including specific RSV clinical isolates and the laboratory engineered RSV A2 strain.

On the other hand, ERNA-RSV-005-GDura showed no substantial activation upon infection of treated cells with viruses from non-RSV viral species such as hMPV, HPIV3, Rhinovirus 60 or EV-D68. Expression level of the GDura polypeptide of interest increased by >3 logs after infection with any of the shown RSV variants compared to the activation level in uninfected cells or in cells infected with a non-RSV virus.

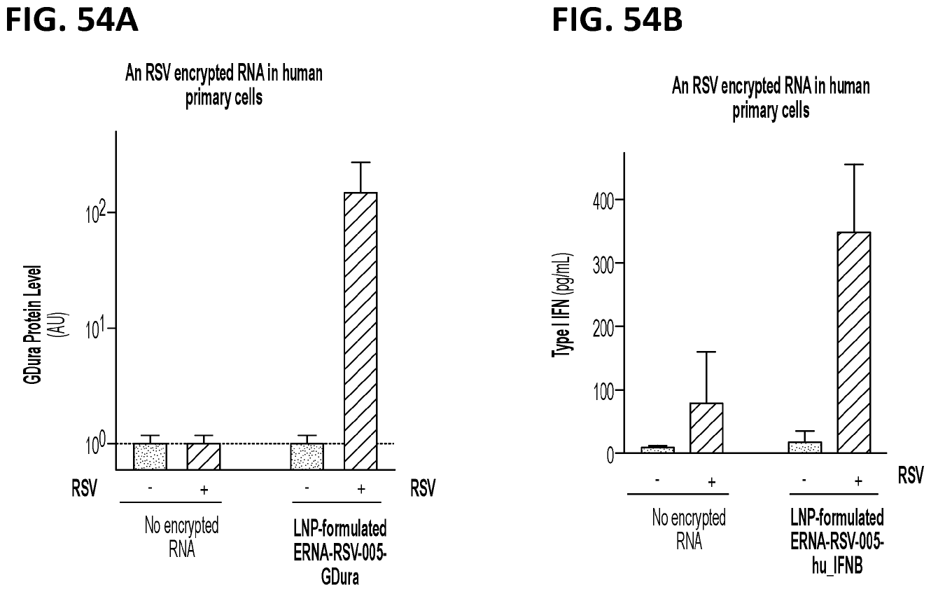

FIGS. 54A-54B show activation of some RSV encrypted RNAs in the presence or absence of RSV infection in human primary airway cells. FIG. 54A shows that human primary airway cells treated with ERNA-RSV-005-GDura exhibited an ~2 log increase in translation of the GDura polypeptide of interest in the presence of RSV infection. FIG. 54B shows an analogous experiment with an RSV encrypted RNA encoding human IFN-β as the polypeptide of interest (ERNA-RSV-005-hu_IFNB), where translation of the therapeutic polypeptide was increased by ~300 μg/ml (as quantified by ELISA) in the presence of RSV infection.

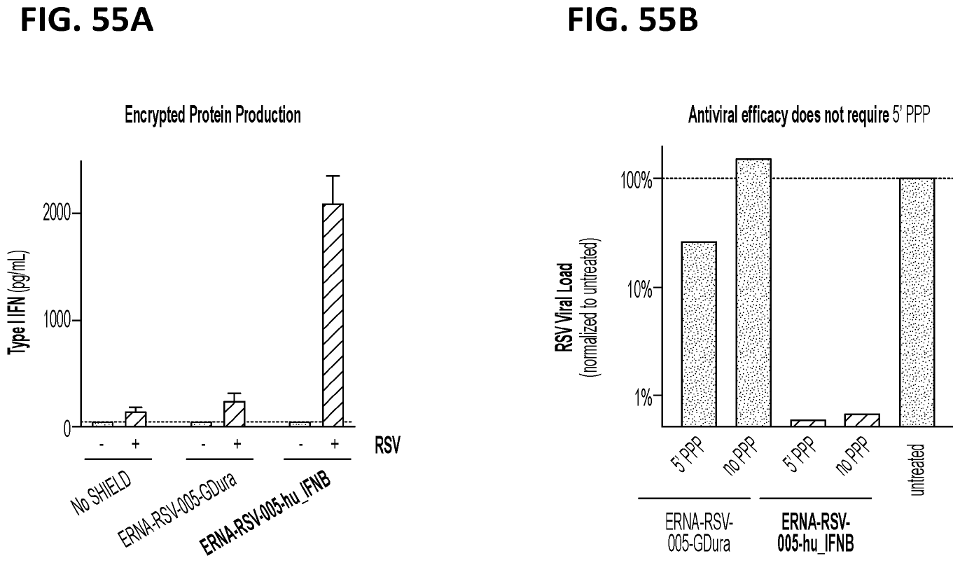

FIG. 55A shows activation of the RSV encrypted RNA encoding human IFN-β in the presence or absence of RSV infection in HEp-2 cells. FIG. 55A shows that translation of the therapeutic polypeptide was increased by ~2000 μg/ml (as quantified by ELISA) in the presence of RSV infection compared to the level of human IFN-β expression in analogously treated uninfected cells or in RSV-infected cells treated with an ERNA-RSV-005-GDura control.

### Example 25: RSV Encrypted RNAs Activated by Viral Infection of Treated Cells Days after Encrypted RNA Treatment

5′-triphosphorylated and 5′-monophosphorylated RSV encrypted RNAs were prepared as described above (e.g., using the ERNA-RSV-001-GDura scaffold in conjunction with the appropriate production plasmid), and then formulated into LNPs as described in Example 9.

At 24 hours before treatment with encrypted RNAs or controls, HEp-2 cells were plated at a concentration of 250,000 cells/well in a 24-well microplates. At −2 hpi, cells were treated with LNP-formulated encrypted RNA or a control substance by dropwise addition to cells to the D02 media and the cultures were allowed to recover in humidified 5% CO₂ incubators at 37° C. A representative dose was treatment with 100 ng or 250 ng of LNP-formulated encrypted RNA (e.g., LMAX-LNP-formulated encrypted RNA).

At ~2 h (0 d), 1 d, 2 d, 3 d, 4 d, 5 d, or >5 d after treatment with an LNP-formulated encrypted RNA or controls, parallel cultures were infected with RSV (e.g., with strain A2) at an MOI of 0, 0.1, or 1. To perform the infection, the culture media was removed and the virus inoculum added in a total volume of 250 μL Opti-MEM per well. Following incubation at 37° C. with 5% CO₂ for ~2 h, the virus inoculum was removed and 250 μL of D02 media was added.

At 24, 48, 72, 120, or >120 hpi, the increase in the translation of the polypeptide of interest (e.g., GDura) in cells treated with an RSV encrypted RNA was quantified relative to the level of the polypeptide of interest in parallel cultures treated with the encrypted RNA but not infected with RSV. To quantify this increase in translation when GDura was the polypeptide of interest, a portion of the culture supernatant (e.g., 10 μL) was collected at multiple time points across the study, and used to quantify the production of secreted luciferase at each time point, using the methods described above.

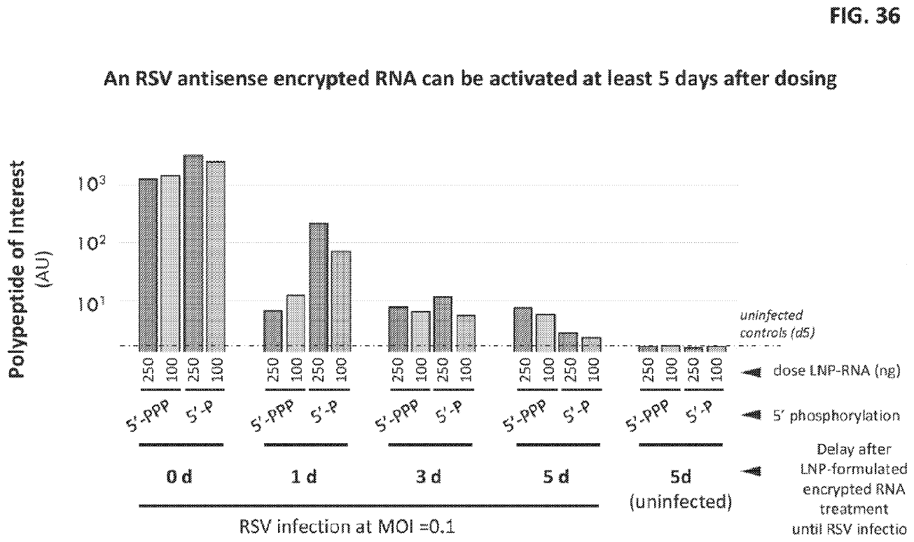

As shown in FIG. 36, 5′-monophosphorylated or 5′-triphosphorylated encrypted RNAs can be activated by RSV infection up to 5 days or more after treatment of the cells with an encrypted RNA, with the level of activation decaying approximately exponentially per the intracellular half-life of the encrypted RNAs.

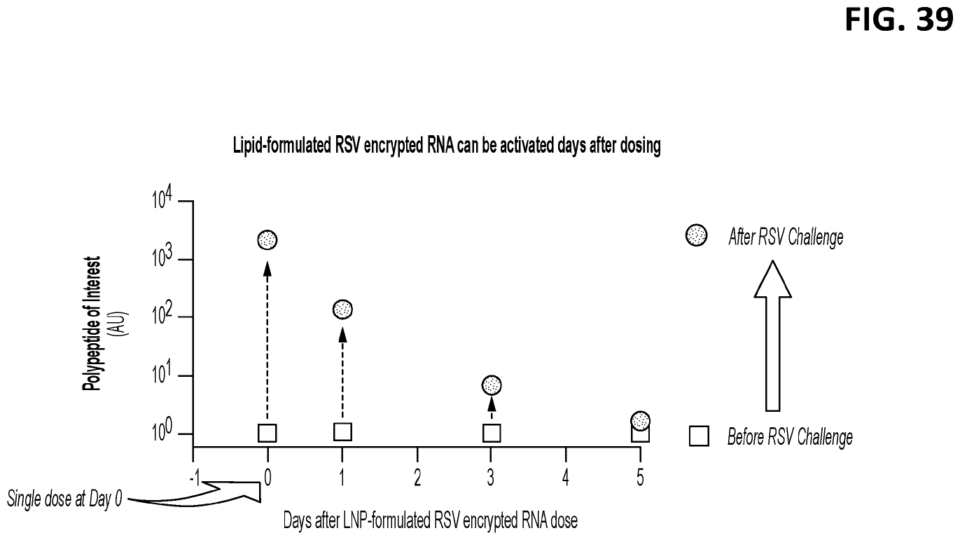

FIG. 39 shows a subset of the data in FIG. 36, and demonstrates the decay over time in the level of encrypted RNA activation when cells were dosed with 250 ng of LNP-formulated 5′-monophosphorylated RSV encrypted RNA (ERNA-RSV-001-GDura) and infected by RSV at either 0, 1, 3, or 5 days post-infection.

### Example 26: Antiviral Activity of RSV Encrypted RNAs

At 24 h before infection, A549-RIGI-KO or 239T cells were plated at a concentration of 400,000 cells/well in a 24-well dish. At 0 hpi, culture media was removed and cells were infected with RSV A2 virus at MOIs of 10, 1, 0.1, or 0 by adding the virus inoculum in a total volume of 250 μL Opti-MEM per well. Following incubation at 37° C. with 5% CO₂ for about 6 h, the virus inoculum was removed and 500 μL of D02 media was added to cells.

LMAX-LNP-formulated encrypted RNAs were prepared as described above in Example 8. LMAX-LNP-formulated encrypted RNAs were added dropwise to cells in D02 media and the cultures allowed to recover in humidified 5% CO₂ incubators at 37° C. Cultures were treated with about 250 ng of LMAX-LNP-formulated encrypted RNA.

When 293T cells were used for the initial infection, virus-containing culture supernatant was collected from the donor infections at about 72 hpi (or hours post-transfection for uninfected cells) and used to infect recipient HEp-2 cells to accurately quantify the viral load via FFU assay. Donor 239T supernatants were transferred to fresh HEp-2 cultures for 2 hours to serve as inocula for infection. After a 2 h incubation, cells were thoroughly washed four times with DPBS to remove any residual carryover of the produced cytokines. Inocula were replaced with 0.5 mL of D02 and the virus infections allowed to proceed and spread for 5 days on HEp-2 cells. After 5 d, the supernatants were collected and analyzed by an RSV FFU assay described above. Viral titers were compared between RSV-infected samples that were treated with a therapeutic RSV encrypted RNA (encoding human IFN-β as the polypeptide of interest) or a non-therapeutic RSV encrypted RNA (encoding GDura as the polypeptide of interest). Equivalent amounts of an mRNA encoding a reporter protein (e.g., RFP) were used as a control.

When A549-RIGI-KO were used for transfection/infections, the supernatants were collected at 5 dpi and the viral titer quantified by RSV FFU assay, as described above. As above, viral titers were compared between RSV infected samples that were treated with an LMAX-LNP-formulated therapeutic RSV encrypted RNA (encoding human IFN-β as the polypeptide of interest) or a control non-therapeutic RSV encrypted RNA (encoding GDura as the polypeptide of interest).

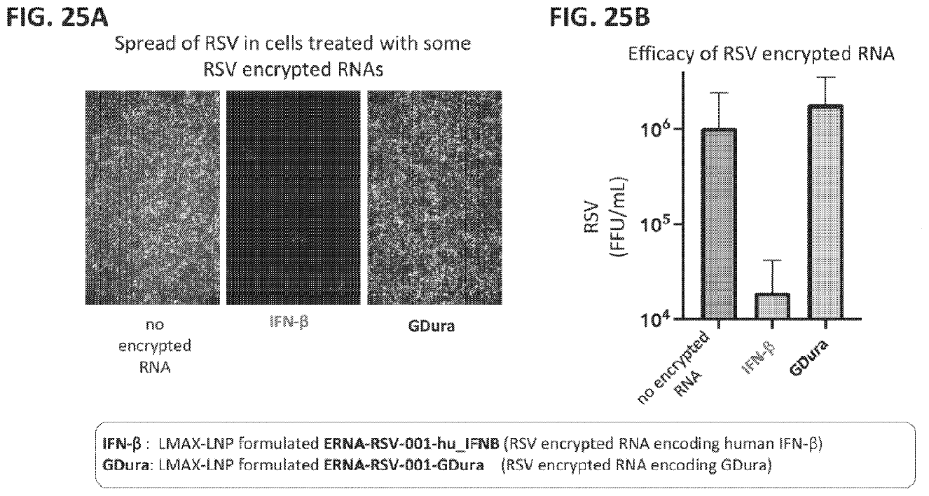

FIG. 25 shows that a therapeutic RSV encrypted RNA encoding human IFN-β as the polypeptide of interest inhibits RSV replication while mock-treatment or treatment of cells with a non-therapeutic RSV encrypted RNA (ERNA-RSV-001-GDura) does not substantially inhibit RSV replication. In the left panel of FIG. 25, micrographs of infections of HEp-2 cells by RSV (labelled with a red fluorescent reporter protein) in the presence or absence of an RSV encrypted RNA show that cells treated with a therapeutic RSV encrypted RNA (encoding human IFN-β as the polypeptide of interest) show a reduction in RSV replication relative to the level of RSV replication in parallel HEp-2 cells treated with a non-therapeutic encrypted RNA (i.e., the identical encrypted RNA scaffold encoding GDura as the polypeptide of interest) or parallel untreated HEp-2 cells. This shows that encrypted RNA efficacy can be payload-driven and is not solely an artifact of cellular immune responses to treatment with an RNA (e.g., an encrypted RNA).

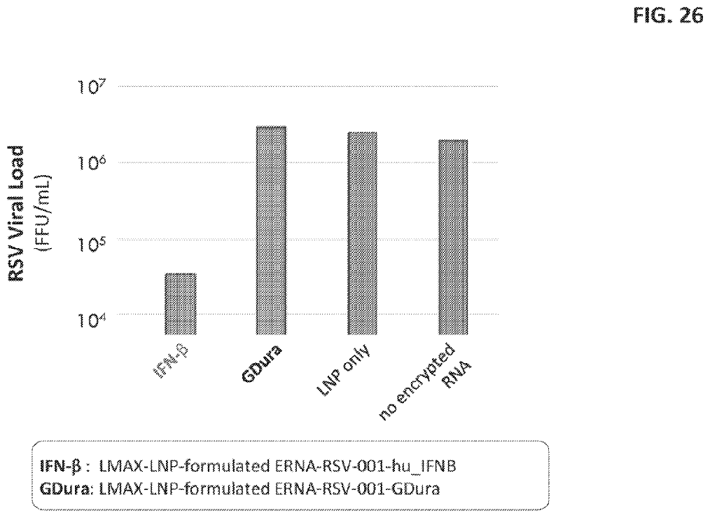

FIG. 26 and the right panel of FIG. 25 show that treatment of HEp-2 cells with a therapeutic RSV encrypted RNA encoding human IFN-β (ERNA-RSV-001-hu_IFNB) can reduce RSV viral titer by approximately 2 log with respect to treatment with negative control articles including: (i) a paired non-therapeutic RSV encrypted RNA encoding GDura as the polypeptide of interest (ERNA-RSV-001-GDura); (ii) LMAX-LNP lipid mix only; or (iii) media only (labelled “no encrypted RNA”).

FIG. 55B shows an approximately 2 log reduction in RSV viral load in Hep-2 cells treated with a therapeutic RSV encrypted RNA encoding human IFN-β protein as the polypeptide of interest (ERNA-RSV-005-hu_IFNB). Notably, the viral load knockdown in comparison to a matched control non-therapeutic encrypted RNA can be even more pronounced when a therapeutic encrypted RNA is optimized to lack a 5′-triphosphate and to thereby reduce off-target immunogenicity.

### Example 27: Prophylactic Efficacy of an LNP-Formulated Encrypted RNA in Mice

Studies of the safety and efficacy of encrypted RNA candidates and controls were performed using laboratory mice. All animal studies were reviewed and approved by independent Institutional Animal Care and Use Committees (IACUC).

### Safety & Tolerability

BALB/c mice (e.g. The Jackson Laboratory; cat. no. 000651) were treated with an LNP-formulated encrypted RNA test article or controls. The test articles or controls were administered as a single 50 μL intranasal dose under an IACUC-approved anesthesia regimen. During intranasal administration under anesthesia, the animal was positioned in a supine position with the dorsal plane aligned to 60° above the horizontal plane (such that head was elevated at an angle approximately 60° above the horizon and the tail lowered to an angle approximately 60° below the horizon). The 50 μL inoculum was instilled dropwise into both nostrils with a sterile micropipette.

After treatment with encrypted RNA or control, animals were weighed daily and closely observed at least once daily using a six-point clinical scale to gauge overall health. Animals were humanely euthanized using an American Vetereniary Medical Association (AVMA) approved method before necropsy and post-mortem tissue collection.

Safety after Treatment with 1 or More Doses of LNP-Formulated Encrypted RNA

Encrypted RNA test articles and control RNAs were each formulated into an LNP suitable for in vivo use, using an approach described in Example 9. Encrypted RNAs tested for in vivo safety included: sarebcovirus encrypted RNAs, influenza encrypted RNAs, and RSV encrypted RNAs.

Groups of mice (at least three per treatment group) were treated intranasally (as described above) with a range of encrypted RNA and control doses. Typical experiments tested the safety of ~0.1-5 μg of an LNP-formulated therapeutic encrypted RNA (test article), ~0.1-5 μg of an LNP-formulated non-therapeutic encrypted RNA control (e.g. an encrypted RNA with the same scaffold as the therapeutic encrypted RNA but encoding a luciferase as the polypeptide of interest), ~0.1-5 μg of an LNP-formulated mRNA control (e.g. encoding a GFP), and an equal volume vehicle-only control (e.g. either PBS alone or PBS+5% sucrose).

After receiving a single dose of the above at day 0, all animals were followed out to 7 days post-treatment. Animal body weights of the mice were measured daily and clinical scores were recorded for each of the animals to quantify signs of disease and any reactions to the LNP-formulated encrypted RNA test articles. For a given treatment dose (e.g. 2 μg), animals treated with a therapeutic encrypted RNA generally did not show significantly enhanced weight loss or deterioration in phenotypic health (quantified by clinical score) over a period of 7 days in comparison to control treated animals. These studies indicated the comparative safety of intranasal treatment of encrypted RNA in healthy animals in comparison to treatment with intranasally delivered RNA controls or vehicle-only controls. Further, the studies demonstrated that an LNP-formulated therapeutic encrypted RNA can be safely delivered intranasally to healthy animals at doses of >1 μg.

### Prophylactic Efficacy of an LNP-Formulated Therapeutic Sarbecovirus Encrypted RNA Against SARS-COV-2 Infection in Mice.

The antiviral efficacy of a therapeutic sarbecovirus encrypted RNA in mice was determined by performing efficacy experiments in which susceptible animals were infected with a SARS-COV-2 variant in the presence or absence of a therapeutic sarbecovirus encrypted RNA and observing any differences in the typical course of infection conferred by the therapeutic sarbecovirus encrypted RNA.

The primary objective of these experiments was to determine if the dosing of mice prophylactically with a therapeutic sarbecovirus encrypted RNA (e.g., ERNA-SARS2-001-m_IFNB) would be sufficient to protect mice from a viral challenge with a lethal SARS-COV-2 variant. A secondary objective was to determine whether mice prophylactically treated with a therapeutic encrypted RNA would have reduced weight loss during disease progression. Taken together, increased survival and prevention of weight loss are strongly correlated with reduced SARS-COV-2 viral load within the lungs. Other indicators of antiviral efficacy include reduced lung pathology or reduced SARS-COV-2 viral load within the lungs.

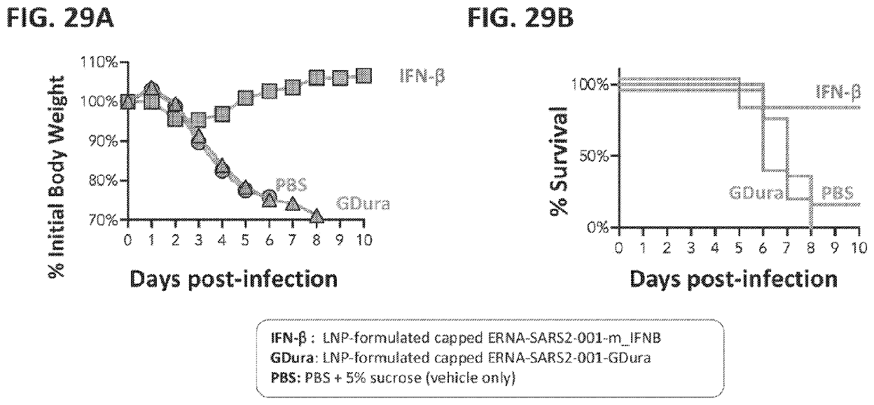

FIG. 29 shows the in vivo prophylactic safety and efficacy of a therapeutic sarbecovirus encrypted RNA (ERNA-SARS2-001-m_IFNB) administered intranasally to mice that were subsequently challenged with a lethal dose of “MA30”, a mouse adapted variant of SARS-COV-2 (SARS2-N501YMA30 in Wong et al., Nature 2022; 605 (7908): 146-151, DOI: 10.1038/s41586-022-04630-3).

Groups of 5 BALB/c mice (6-10 weeks old) were treated with one of 3 experimental interventions as a single 50 μL intranasal dose, either: 2 μg of an LNP-formulated therapeutic sarbecovirus encrypted RNA (ERNA-SARS2-001-m_IFNB) in PBS containing 5% sucrose (test article), 2 μg of an LNP-formulated non-therapeutic sarbecovirus encrypted RNA (ERNA-SARS2-001-GDura) in PBS containing 5% sucrose (control or sham encrypted RNA), or PBS containing 5% sucrose (vehicle-only control). Notably, the same LNP composition was used to formulate the therapeutic encrypted RNA (encoding mouse interferon-beta as the polypeptide of interest) and the control encrypted RNA (encoding a luciferase as the polypeptide of interest). We also note that mouse interferon-beta was encoded in the test article, given the requirement for a species specific interferon-beta (e.g. due to differences in interferon receptors between humans and mice).

At 1 day post-treatment, all mice were intranasally challenged with ~3,000 PFU of MA30. In 6-10 week old BALB/c mice, 3000 PFU of MA30 is ~5× higher than the lethal dose that, on average, induces a lethal infection in 50% of infected animals (LD₅₀). A challenge dose of 3000 PFU of MA30 is therefore generally lethal in ~100% of these mice within ~6-8 days after infection (in the absence of effective antiviral treatment or prophylaxis). The body weights, appearance, behavior, and survival of all of the mice were monitored over a period of 10 days post-infection or until a percentage (25% or more) of body weight loss or deteriorating clinical scores necessitated euthanasia in accordance with IACUC and ethical standards.

The left panel of FIG. 29 shows mean body weight measurements for each treatment or control group across the study. The right panel of FIG. 29 shows Kaplan-Meier survival curves for each group. Treatment with the therapeutic sarbecovirus encrypted RNA substantially increases both mean body weight recovery and % survival after MA30 challenge. When prophylactically treated with the therapeutic sarebcovirus encrypted RNA, 4/5 animals (80%) recovered body weight and phenotypic health after the MA30 viral challenge. In contrast, only 1/10 control-treated animals recovered body weight and survived. In particular, all mice treated with the non-therapeutic sarbecovirus encrypted RNA (ERNA-SARS2-001-GDura) lost >30% of their weight within 6-8 days and succumbed to infection.

FIG. 29 demonstrates that a therapeutic sarbecovirus encrypted RNA, when administered prophylactically, can provide substantial protection against lethal sarbecovirus infections in vivo- and that such protection can require translation of a therapeutic protein by the therapeutic encrypted RNA. We note that the control encrypted RNA and the therapeutic encrypted RNA used in this study share the same encrypted RNA scaffold (ERNA-SARS2-001-XX), the same LNP formulation, and the same PBS+5% sucrose vehicle. The key difference between treatment and “sham” control articles is that the treatment article encoded a therapeutic polypeptide as the polypeptide of interest.

### Example 28: A Sarbecovirus Encrypted RNA Scaffold can be Used to Protect Against Sarbecoviral Infections in Mice by Encoding Different Therapeutic Polypeptides of Interest

In Example 27, a therapeutic sarbecovirus encrypted RNA encoding mouse interferon-beta as the polypeptide of interest was shown to confer antiviral protection against lethal sarbecovirus infections in vivo. To additionally test whether the same therapeutic sarebcovirus encrypted RNA scaffold could encode different therapeutic polypeptides of interest (beyond an interferon-beta) and still confer antiviral protection against lethal sarbecovirus infections in vivo, a Type III interferon (a mouse interferon lambda) was encoded as the polypeptide of interest in a follow-on experiment. More specifically, the antiviral efficacy of LNP-formulated ERNA-SARS2-001-m_IFN_lambda_2 was tested in a similar prophylactic study to that shown in Example 27 (the only additional difference in study design is that treatment occurred one day earlier, at 48 h prior to infection, in Example 28). This follow-on study was performed to test the plug-and-play capability of an encrypted RNA scaffold to encode arbitrary therapeutic polypeptides of interest. Taken together with Example 27, Example 28 shows that a single encrypted RNA scaffold with a specific L region sequence and R region sequence can be used to encode different therapeutic polypeptides of interest, and that each resulting therapeutic encrypted RNA can remain therapeutically protective in vivo.

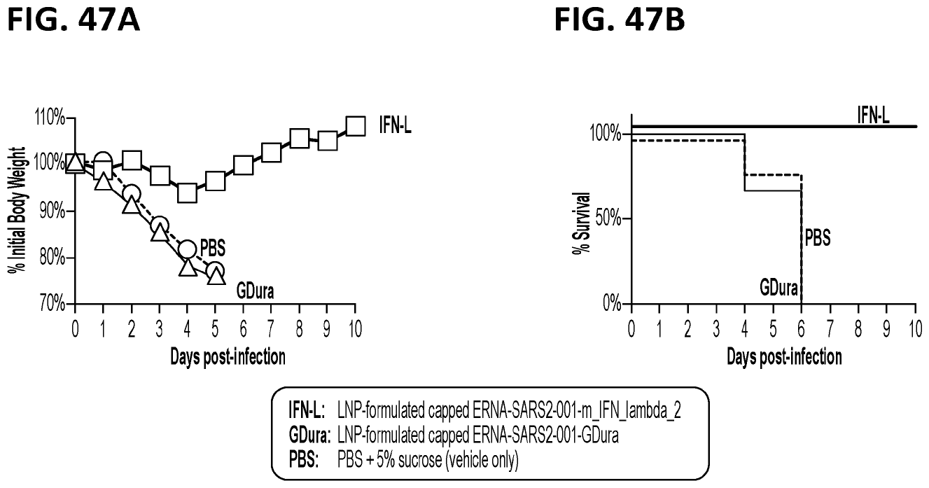

FIG. 47 shows that a therapeutic sarbecovirus encrypted RNA encoding an interferon-lambda as the polypeptide of interest remains protective against a SARS-CoV-2 variant (MA30) in a lethal infection in animals. Groups of 5 BALB/c mice (6-10 weeks old) were treated with one of 3 experimental interventions as a single 50 μL intranasal dose, either: 2 μg of an LNP-formulated therapeutic sarbecovirus encrypted RNA (ERNA-SARS2-001-m_IFN_lambda_2) in PBS containing 5% sucrose (test article), 2 μg of an LNP-formulated non-therapeutic sarbecovirus encrypted RNA (ERNA-SARS2-001-GDura) in PBS containing 5% sucrose (control or sham encrypted RNA), or PBS containing 5% sucrose (vehicle-only control). The same LNP composition was used to formulate the therapeutic sarbecovirus encrypted RNA (encoding mouse interferon-lambda 2 as the polypeptide of interest) and the control non-therapeutic sarbecovirus encrypted RNA (encoding a luciferase as the polypeptide of interest). At 2 days post-treatment, all mice were intranasally challenged with ~3,000 PFU of MA30, and the animals were monitored for 10 days post-infection as in Example 27.

FIG. 47 shows analogous body weight (left panel) and Kaplan-Meier survival (right panel) curves to those shown in FIG. 29. Notably, while 5 of 5 mice prophylactically treated with the therapeutic sarbecovirus encrypted RNA (ERNA-SARS2-001-m_IFN_lambda_2) were protected from lethal infection, the mice treated with the control non-therapeutic sarbecovirus encrypted RNA (i.e. the encrypted RNA scaffold encoding a luciferase) were not protected. 5 of 5 (100%) mice treated with the non-therapeutic sarebcovirus encrypted RNA succumbed to the SARS-COV-2 infection within 6 days. This demonstrates that a therapeutic encrypted RNA can confer efficacy in vivo by encoding different therapeutic polypeptides of interest.

### Example 29: An RSV Encrypted RNA Encoding a Therapeutic Polypeptide of Interest is not Protective Against a SARS-COV-2 Infection in Mice

In Examples 27 and 28, encoding a therapeutic polypeptide of interest was necessary for a sarbecovirus encrypted RNA to confer antiviral protection against lethal sarbecovirus infections in vivo (i.e. a control sarbecovirus encrypted RNA encoding a luciferase as the polypeptide of interest did not confer protection in vivo in those studies). To additionally test whether encoding a therapeutic polypeptide of interest can be sufficient for an encrypted RNA to confer antiviral protection against lethal sarbecovirus infections in vivo even when encoded by an encrypted RNA not activated by sarbecoviruses, a therapeutic polypeptide of interest (a mouse interferon lambda) was encoded in a non-sarbecovirus encrypted RNA. More specifically, a therapeutic RSV encrypted RNA (ERNA-RSV-005-m_IFN_lambda_2) was LNP formulated using the same LNP as used in FIGS. 29 and 47 and administered to mice intranasally.

FIG. 48 shows that the therapeutic RSV encrypted RNA does not protect against a SARS-COV-2 variant (MA30) in a lethal infection in animals. Groups of 5 BALB/c mice (6-10 weeks old) were treated with one of 3 experimental interventions as a single 50 μL intranasal dose, either: 2 μg of the LNP-formulated therapeutic sarbecovirus encrypted RNA (ERNA-SARS2-001-m_IFN_lambda_2) in PBS containing 5% sucrose (test article), 2 μg of the LNP-formulated therapeutic RSV encrypted RNA (ERNA-RSV-005-m_IFN_lambda_2) in PBS containing 5% sucrose (control or sham encrypted RNA), or PBS containing 5% sucrose (vehicle-only control). The same LNP composition was used to formulate the therapeutic sarbecovirus encrypted RNA (encoding mouse interferon-lambda 2 as the polypeptide of interest) and the control therapeutic RSV encrypted RNA (encoding the same mouse interferon-lambda 2 protein as the polypeptide of interest). As in Example 27, at 1 day post-treatment, all mice were intranasally challenged with ~3,000 PFU of MA30, and monitored for 10 days post-infection.

FIG. 48 shows analogous body weight (left panel) and Kaplan-Meier survival (right panel) curves to those shown in FIGS. 29 and 47. Notably, while 5 of 5 mice prophylactically treated with the therapeutic sarbecovirus encrypted RNA are protected from lethal infection, the mice treated with the therapeutic RSV encrypted RNA (i.e. an encrypted RNA not activated by a sarbecovirus) are not generally protected. 4 of 5 (80%) mice treated with the therapeutic RSV encrypted RNA succumb to the SARS-COV-2 infection within 7 days. This demonstrates an example in which a therapeutic polypeptide of interest is not sufficient for an encrypted RNA to confer protection against lethal sarbecoviruses infections. In this experiment, the therapeutic polypeptide of interest was only protective when encoded by a sarbecovirus encrypted RNA (i.e. by an encrypted RNA activated by MA30). As above in Example 20, this further demonstrates that an encrypted RNA can be used to limit the activity of therapeutic proteins (e.g. immunomodulatory proteins) absent infection of a subject with viruses that encode a translation activator of the encrypted RNA.

### Example 30: Therapeutic Efficacy of an LNP-Formulated Encrypted RNA in Mice

In Examples 27-29, prophylactic treatment with a therapeutic sarbecovirus encrypted RNA was shown to confer antiviral protection against lethal sarbecovirus infections in vivo. To additionally test whether the therapeutic sarebcovirus encrypted RNA would be protective if administered therapeutically (i.e., after infection with SARS-COV-2 MA30), a similar experiment to those described in Examples 27-29 was performed, albeit with treatment administered 6 h after infection with a lethal dose of MA30.

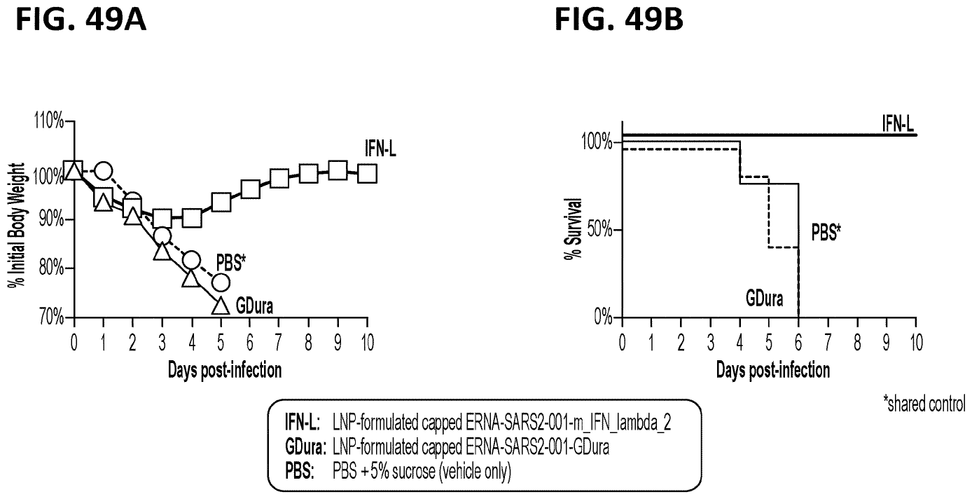

FIG. 49 shows that a therapeutic sarbecovirus encrypted RNA encoding an interferon-lambda is protective even when administered after the animals are first challenged with a lethal dose of a SARS-COV-2 variant (MA30). All mice in this study were intranasally challenged with ~3,000 PFU of MA30. At 6 h post-infection, groups of 5 BALB/c mice (6-10 weeks old) mice were treated with one of 2 experimental interventions as a single 50 μL intranasal dose, either: 2 μg of an LNP-formulated therapeutic sarbecovirus encrypted RNA (ERNA-SARS2-001-m_IFN_lambda_2) in PBS containing 5% sucrose, or 2 μg of an LNP-formulated non-therapeutic sarbecovirus encrypted RNA (ERNA-SARS2-001-GDura) in PBS containing 5% sucrose (control or sham encrypted RNA). The study was performed contemporaneously with the study in Example 28 (FIG. 47), so an additional shared control group of 5 BALB/c mice (6-10 weeks old) was monitored consisting of mice intranasally treated 2 days prior to infection with a single 50 μL intranasal dose PBS containing 5% sucrose (vehicle-only control). The same LNP composition was used to formulate the therapeutic sarbecovirus encrypted RNA (encoding mouse interferon-lambda 2 as the polypeptide of interest) and the control non-therapeutic sarbecovirus encrypted RNA (encoding a luciferase as the polypeptide of interest). All animals were monitored for 10 days post-infection as in Examples 27-29.

FIG. 49 shows analogous body weight (left panel) and Kaplan-Meier survival (right panel) curves to those shown in FIG. 29. Notably, while 5 of 5 mice therapeutically treated post-infection with the therapeutic sarbecovirus encrypted RNA (ERNA-SARS2-001-m_IFN_lambda_2) were protected from lethal infection, mice treated with the control non-therapeutic sarbecovirus encrypted RNA (i.e. the same encrypted RNA scaffold encoding a luciferase) were not protected. 5 of 5 (100%) mice treated with the non-therapeutic sarbecovirus encrypted RNA succumbed to the SARS-COV-2 infection within 6 days. This demonstrates that a therapeutic encrypted RNA can confer efficacy in vivo, when first administered after viral infection.

### Example 31: Safety and Efficacy of an LNP-Formulated Encrypted RNA in Hamsters

Studies of the safety and efficacy of encrypted RNA candidates and controls were also performed using laboratory hamsters. All hamster studies were reviewed and approved by an independent IACUC.

### Safety & Tolerability

To test the safety of LNP-formulated encrypted RNAs in an additional animal model, healthy ~8-10 week-old Syrian hamsters (e.g. Charles River Laboratories International, Inc., Strain 049) were treated with therapeutic encrypted RNA candidates or controls via a single intranasal 100 μL dose under an IACUC-approved anesthesia regimen.

Groups of hamsters were treated intranasally with a range of encrypted RNA and control doses. Typical experiments tested the safety of a single intranasal instillation of either: ~5-8 μg of an LNP-formulated therapeutic encrypted RNA (test article), ~5-8 μg of an LNP-formulated non-therapeutic encrypted RNA control (e.g. an encrypted RNA with the same scaffold as the therapeutic encrypted RNA, but encoding a luciferase as the polypeptide of interest), ~5-8 μg of an LNP-formulated mRNA control (e.g. encoding a GFP), or an equal volume vehicle-only control (e.g. PBS+5% sucrose).

After receiving a single dose of the above at day 0, all animals were followed out to 3 days post-treatment. Animal body weights of the mice were measured daily and clinical scores were recorded for each of the animals to quantify signs of disease and any reactions to the LNP-formulated encrypted RNA test articles. For a given treatment dose (e.g. 8 μg), animals treated with a therapeutic encrypted RNA generally did not show enhanced weight loss or deterioration in phenotypic health (quantified by clinical score) in comparison to control treated animals. These studies indicated the safety of intranasal treatment of encrypted RNA in healthy hamsters in comparison to treatment with intranasally delivered RNA controls or vehicle-only controls. Further, the studies demonstrated that an LNP-formulated therapeutic encrypted RNA can be safely delivered intranasally to healthy animals at doses of >5 μg.

### Safety & Efficacy of an LNP-Formulated Therapeutic Sarbecovirus Encrypted RNA During Treatment of SARS-COV-2 in Hamsters.

The above safety studies were performed in the absence of virus infection. However, the possibility exists that the delivery of an LNP-formulated encrypted RNA to the lungs in conjunction with SARS-COV-2 infection could potentially exacerbate the lung damage that the virus is known to cause. For this reason, we designed a combined safety-efficacy study to test the effects of LNP-formulated encrypted RNAs in hamsters that were also infected SARS-COV-2.

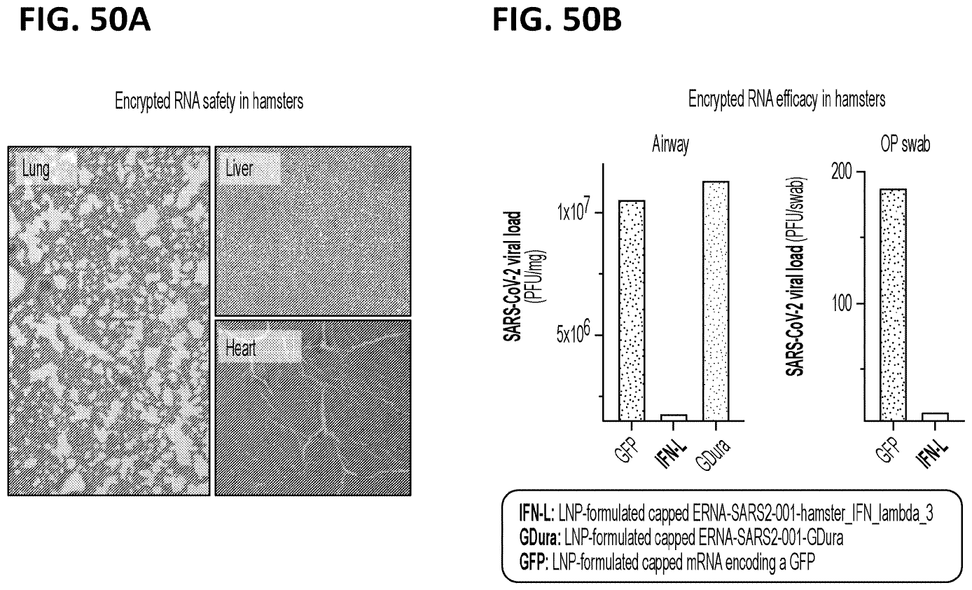

FIG. 50A shows the safety of a prophylactic administration of a therapeutic sarbecovirus encrypted RNA in both uninfected hamsters (heart and spleen images) and hamsters subsequently infected with SARS-COV-2 (lung image).

Groups of 4 animals were anesthesized and treated with an intranasal instillation of 100 μL containing either: 8 μg of an LNP-formulated therapeutic sarbecovirus encrypted RNA (ERNA-SARS2-001-hamster_IFN_lambda_3), 8 μg of an LNP-formulated non-therapeutic sarbecovirus encrypted RNA (ERNA-SARS2-001-GDura), 8 μg of a control LNP-formulated mRNA (encoding a GFP), or an equal volume vehicle-only control (e.g. PBS+5% sucrose). The therapeutic encrypted RNA and control RNAs were formulated using the same LNP composition. At 1 day post-treatment, each group of animals was infected via intranasal challenge with 104 PFU of SARS-COV-2 variant WA1.

Body weight and clinical scores were measured in all animals from −24 hpi to +72 hpi. Oropharyngeal swabs were collected at +72 hpi to quantify viral load in the oropharynx at +72 hpi. After swab collection, all study animals were euthanized by an AVMA-approved method at the conclusion of the study (+72 hpi). Terminal tissue collection included airway target tissues (nasal turbinates, cranial lung) and off-target tissues (liver, spleen, heart).

In parallel, the safety of encrypted RNA delivery was also evaluated in uninfected hamsters. Animals were anesthesized and treated, as above, with an intranasal instillation of 100 μL containing either: 8 μg of an LNP-formulated therapeutic sarbecovirus encrypted RNA (ERNA-SARS2-001-hamster_IFN_lambda_3), 8 μg of an LNP-formulated non-therapeutic sarbecovirus encrypted RNA (ERNA-SARS2-001-GDura), 8 μg of a control LNP-formulated mRNA (encoding a GFP), or an equal volume vehicle-only control (e.g. PBS+5% sucrose). The therapeutic encrypted RNA and control RNAs were formulated using the same LNP composition. Notably, the animals were not subsequently infected with SARS-COV-2 in this study. All uninfected animals were monitored for body weight changes and clinical score for 72 h. After 72 h, the animals were euthanized, and samples of airway target tissues (nasal turbinates, cranial lung) and off-target tissues (liver, spleen, heart) were collected as above at necroscopy.

In uninfected animals, trachea, heart, liver, and spleen were normal in all treatment groups. Similarly, lung sections of all treated animals were scored as either normal (50% had no pathology present) or were assigned the lowest grade of minor inflammation (the remaining 50%). Low grade, minor inflammation can be observed with the administration of any agent to the airway.

As with uninfected animals, there were no statistically significant differences in body weight between animals treated with the therapeutic encrypted RNA and animals treated with control, when the animals were subsequently challenged with virus (two sample Student's t-test, p>0.05, on each day of daily weight comparisons).

In addition to the holistic clinical scoring, select organs were obtained at necropsy, then sectioned, H&E-stained, and histologically examined for signs of toxicity by a veterinary pathologist. In all animals, tracheal sections were examined for inflammation and vasculitis while lung sections were additionally evaluated for bronchitis, alveolitis, and hyperplasia.

FIG. 50A shows H&E stained lung, heart, and liver samples. Notably, the lung sample is from an infected animal, while the heart and liver samples were from the matched safety studies (with the same treatment dose) when animals were not subsequently infected with SARS-COV-2. The pathologist found the tissues to be essentially normal, indicating the safety of encrypted RNA treatment.

FIG. 50B shows the antiviral efficacy of the therapeutic encrypted RNA, as quantified by viral load reduction in infected hamsters. Infectious viral loads were quantified from oropharyngeal swabs (OP swabs) and airway tissue (nasal turbinates) collected at 72 hpi by performing plaque assays on homogenates of these samples.

Animals treated with ERNA-SARS2-001-hamster_IFN_lambda_3 had substantially lower viral loads in the oropharynx (median viral load reduction of ~1 log) compared with animals treated with the control encrypted RNA (encoding GDura) or the control mRNA encoding a GFP. Similarly, animals treated with ERNA-SARS2-001-hamster_IFN_lambda_3 had substantially lower viral loads in airway nasal turbinates (80% decrease or 4× reduction in viral load) compared to animals treated with the control mRNA encoding a GFP.

Thus, treatment with ERNA-SARS2-001-hamster_IFN_lambda_3 has an antiviral effect (as quantified by reduced viral loads) and can provide an additional clinical benefit via the reduction of viral replication and inflammation in the airway (both in nasal turbinates and in the lungs).

Taken together, the studies in this example, including those shown in FIGS. 50A-50B, demonstrate the in vivo safety and efficacy of a therapeutic sarbecovirus encrypted RNA in both uninfected hamsters and in hamsters infected with SARS-CoV-2.

### Example 32: Treatment of Animals with DNA-Encoded Encrypted RNA Cassettes Delivered Via Lentiviral Vector

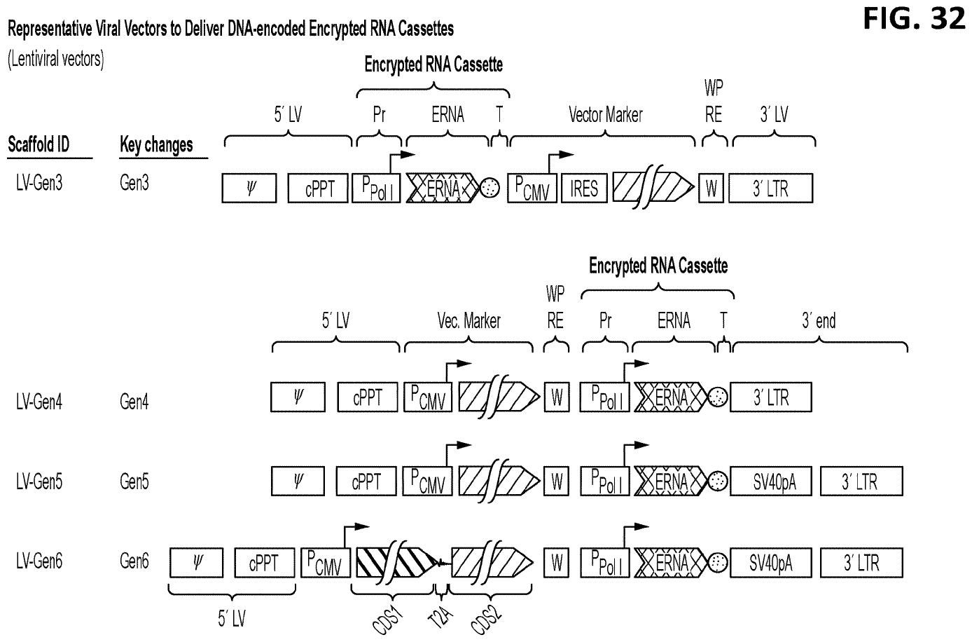

Lentiviral vectors (LV) encoding the DNA-encoded encrypted RNA cassettes from pAT002-ERNA-IAV-002-GDura and pAT002-ERNA-IAV-002-m_IFNB were prepared and VSV-G pseudotyped, as described above. Separate preparations of lentiviral vectors using pLVG04-AT002-ERNA-IAV-002-GDura or pLVG04-AT002-ERNA-IAV-002-m_IFNB were produced. For simplicity, in this Example, LVG04-ERNA-GDura means lentiviral vectors prepared with pLVG04-AT002-ERNA-IAV-002-GDura and likewise LVG04-ERNA-m_IFNB means lentiviral vectors prepared with pLVG04-AT002-ERNA-IAV-002-m_IFNB. These lentiviral vectors are represented in a simplified schematic in FIG. 32.

BALB/cJ mice were treated intranasally with lentiviral vector test articles using the same methodology explained in Example 27. For treatment, groups of 3 to 4 mice received one of the following four test articles intranasally: (i) 10⁷ vg of LVG04-ERNA-GDura (VSV-G pseudotyped); (ii) 107 vg of LVG04-ERNA-m_IFNB (VSV-G pseudotyped); (iii) 107 vg of heat-inactivated (30 min at 65° C.) LVG04-ERNA-GDura (VSV-G pseudotyped) to serve as a non-infectious control; (iv) vehicle-only control comprised of D10 media.

General condition and health parameters (e.g. weight, activity) were observed and monitored daily. Groups of mice were followed out for 28 days after receiving a single dose of a lentiviral vector encoding an encrypted RNA or a control. No differences in body weight and overall health were observed among the 4 groups, demonstrating the safety and tolerability of a DNA-encoded encrypted RNA treatment.

In addition, groups of BALB/c mice (N=3) were treated with preparations of lentiviral vector LVG07-ERNA-SARS1-GDura or PBS control intranasally (1E7 transducing units of lentiviral vector) and monitored for a period of 3 days to 10 days post-treatment. At 3 days and 10 days post-treatment, animals were euthanized and lung RNA isolated. The level of ERNA-SARS1-GDura RNA, emanating from the lentiviral provirus within transduced tissue, was quantified by RT-qPCR as described above. Detectable levels of encrypted RNA were observed within the lungs of treated mice, and no encrypted RNA was detectable in PBS-control treated mice (both at 3 days and 10 days post-treatment).

### Example 33: (Prophetic) Engineering of Cells to Harbor a DNA-Encoded Encrypted RNA Cassette or an Encrypted RNA

Using DNA-encoded encrypted RNA cassettes such as those developed in the above Examples, it is possible to develop a “knock-in” vector, which would add at least flanking homology arms to a DNA-encoded encrypted RNA cassette to direct integration of the DNA-encoded encrypted RNA cassette into a suitable genomic location, such as a safe-harbor locus (e.g. ROSA26), and other key genetic elements to facilitate selection of cells harboring the DNA-encoded encrypted RNA cassette. Non-limiting methods to improve knock-in success rates include: cleaving the genome within the integration locus by CRISPR-assisted integration (e.g., co-administration of a Cas9 with a suitable guide RNA), cleaving the integration site with a TALEN or zinc-finger nuclease to assist in homology directed repair, using a transposon, or using a site-specific recombinase (e.g. phiC31 integrase or Cre recombinase). An alternative procedure would be to introduce a DNA-encoded encrypted RNA cassette via treatment of cells with a viral vector, such as a lentiviral vector. Encrypted RNA could also be applied directly to a host cell, for example, using an LNP formulation or via electroporation.

Encrypted RNAs or DNA-encoded encrypted RNA cassettes could encode a therapeutic polypeptide of interest or a non-therapeutic polypeptide of interest. Some therapeutic polypeptides of interest can be antiviral, for example: immunomodulatory proteins (e.g. interferon proteins), dominant negative proteins, antibody proteins, or other effective therapeutic proteins.

Any of the above procedures, or combinations thereof, could be iteratively performed to yield cells harboring one or more independent DNA-encoded encrypted RNA cassettes.

Some therapeutic polypeptides of interest may be more effective when a host cell possesses certain alleles than when a host cell lacks certain alleles. As an example, some therapeutic polypeptides of interest, such as interferon proteins, rely on the induction of host genes (e.g., interferon-stimulated genes) for efficacy. Thus, a host cell that possesses certain alleles (such as a functional interferon-simulated gene) may have improved antiviral efficacy when treated with a therapeutic encrypted RNA in comparison to a host cell that lacks these alleles.

Conversely, some therapeutic polypeptides of interest may be less effective when a host cell possesses certain alleles than when a host cell lacks these alleles.

### Application to Particular Host Cells

Non-limiting examples of host cells include: primary cells obtained from solid organs (e.g. hepatocytes from a liver) or from collections of blood (lymphocytes from whole blood); pluripotent or totipotent cells; primordial germ cells; reprogrammed cells; homogeneous cell lines; heterogeneous cell lines; or transformed cell lines.

When an encrypted RNA is introduced to a host cell as a DNA-encoded encrypted RNA cassette, the integration of a DNA-encoded encrypted RNA cassette into the host genome may occur episomally or may be chromosomal. When integration is chromosomal, integration can occur at a specific locus (e.g., ROSA26) or occur approximately randomly (e.g. via lentiviral vector transduction).

Many cells support integration into and robust expression from “safe-harbor” loci, such as ROSA26, AAVS1, or CCR5 loci. As non-limiting examples, there are demonstrated integrations of transgenes into safe-harbor loci, including the ROSA26 loci of: rodents (Chu et al., BMC Biotechnology (2016); DOI: 10.1186/s12896-016-0234-4); pigs (Rieblinger et al., PNAS (2021); DOI: 10.1073/pnas.2022562118); cows (Wang et al, Sci. Reports (2018); DOI: 10.1038/s41598-018-28502-x); or sheep (Wu et al., Sci Reports (2016); DOI: 10.1038/srep24360). Cells from some avian species, such as the domestic chicken, support robust expression from an inserted transgene when integration occurs within an endogenous attP locus (Rieblinger et al., PNAS (2021); DOI: 10.1073/pnas.2022562118). Cells from some fish species, such as zebrafish (Kimura et al., Sci Reports (2014); DOI: 10.1038/srep06545) and salmonids (e.g. AquAdvantage salmon) (see Federal Register, Vol 80, 73104-73105 or FDA NADA 141-454), support robust expression from inserted transgenes at a variety of genomic locations.

Plant cells, including those of important agricultural species, such as rice (Lu et al, Nat Biotech (2020); DOI: 10.1038/s41587-020-0581-5), wheat, corn, grapes, cabbage, brassicas (reviewed in (Loyola-Vargas et al., Methods in Molecular Biology (2018); DOI: 10.1007/978-1-4939-8594-4_7)) support transgenic insertions with a variety of methods, including CRISPR/Cas.

### Some Considerations for the Genetic Engineering of Encrypted RNAs in Animal Cells

Some animals (and the cells derived from these animals) are deficient in one or more immune pathays that are required for protection via an immunomodulatory protein. Thus, if an encrypted RNA encodes an immunomodulatory protein as the polypeptide of interest, treated animals (or animal cells) would likely not be protected from a targeted disease if the polypeptide of interest relies on an immune pathway that is deficient in the treated animals. To enable encrypted RNA efficacy in such cases, the deficient immune pathway would also need to be restored in treated animal cells (e.g. by encoding a functional form of the deficient immune protein in a lentiviral vector that also delivers the encrypted RNA).

For example, strains of inbred laboratory mice (e.g., BALB/c, C57BL/6) are often homozygous for a non-functional Mx1 gene, often lacking exons 9-11 with respect to the Mx1 from mouse A2G Mx1 (Ferris et al, 2013; PLOS Pathogens; DOI: 10.1371/journal.ppat.1003196). Mx1 is an interferon stimulated gene that helps confer protection against influenza infection (Haller et al., 2007; Microbes and Infection; DOI: 10.1016/j.micinf.2007.09.010). Crucially, commonly used mouse-derived cell lines and primary mouse cells are frequently derived from Mx1-deficient mice and are therefore deficient in a robust innate immune response against influenza infection (e.g., Let1a airway epithelial cells from C57BL/6; NIH 3T3 fibroblasts from an NIH/Swiss mouse embryo).

Thus, if cells without a functional Mx1 are used, it may be necessary to correct the Mx1 deficiency, either by replacement of the deficient alleles with functional Mx1 or via introduction of a synthetic Mx1 paralog. Addition of a paralogous allele could be accomplished via insertion of a copy of the Mx1 coding sequence under control of an interferon-stimulated promoter. Introduction of such an Mx1 cassette could occur before editing with an encrypted RNA cassette, simultaneous with editing, or after addition of an encrypted RNA cassette.

To test the antiviral effect of modifying a cell line via insertion of a DNA cassette encoding a therapeutic encrypted RNA, at least two new isoclonal cell lines should be created in parallel from the same progenitor population. One cell line will contain a putatively effective therapeutic influenza encrypted RNA cassette while the other cell line will contain a putatively non-effective influenza encrypted RNA cassette but otherwise contain the same flanking effective genetic elements. A representative example of a pair of cell lines would be a cell line encoding a therapeutic encrypted RNA encoding for mouse IFN-β and a cell line encoding an influenza encrypted RNA encoding a luminescent or fluorescent reporter protein (expected not to be antiviral).

After generation of the cell lines, transcription of the encrypted RNA from the DNA cassette introduced into the cell line would be confirmed by RT-qPCR, as described above. Similar to the results detailed in Example 15 above, influenza infection of the cell line harboring the therapeutic influenza encrypted RNA cassette would be expected to have reduced levels of influenza replication (e.g., at least 1 log reduction) in comparison to the “sham-treated” cell line harboring the non-therapeutic influenza encrypted RNA cassette.

In an optional step, the encrypted RNA cassette would be inactivated (by deletion, insertion, or other effective procedure to genetically inactivate the encrypted RNA cassette) and would be shown to restore influenza replication to approximately the levels measured in the sham-treated cell line. The purpose of this prospective manipulation is to show that the antiviral effect is mediated by the therapeutic encrypted RNA and is not due to an inadvertent off-target “hitchhiker” mutation that is genetically linked to insertion of the effective DNA cassette encoding an encrypted RNA.

### Example 34: (Prophetic) Production of a Transgenic Mouse with Germline Production of an Encrypted RNA

Using an effective transgene cassette validated in Example 33, a mouse strain suitable for both influenza infection and germline manipulation (e.g., C57BL/6 wildtype with a non-functional Mx1) will be utilized as the recipient animal for transgenesis. Standard methods could be used to generate transgenic knock-in mice, including by microinjection of mouse zygotes with DNA harboring the encrypted RNA cassette and coinjection with Cas9 mRNA and sgRNA targeting Rosa26 (Chu et al., BMC Biotechnology, 2016, DOI: 10.1186/s12896-016-0234-4).

As a DNA-encoded encrypted RNA cassette has decreased translation of the polypeptide of interest in the absence of a translation activator, we anticipate that embryo survival will be high. This is because production of the polypeptide of interest is expected to be low in the embryonic state due to the absence of a translation activator. After rearing pups, standard backcrossing methods could be used to generate animals that are either heterozygous or homozygous for the encrypted RNA cassette. Validation for encrypted RNA expression and activation will occur via standard methods, such as RT-qPCR or RNA FISH of various tissues.

In the case of germline modification with a DNA-encoded therapeutic influenza encrypted RNA, the DNA encoding the encrypted RNA cassette will also encode a functional Mx1. Functional tests for translation activation of the influenza encrypted RNA cassette by influenza infection can occur by isolating permissive cells, infecting with influenza, and measuring the subsequent changes in levels of the polypeptide of interest. The specificity of this effect can be measured as in Example 13, where infection with off-target viruses is not expected to result in enhanced protein translation. If the polypeptide of interest were a suitable protein (e.g. a fluorescent or luminescent protein), infection could be closely monitored by measuring the level of the protein using a convenient and sensitive monitoring method.

### Example 35: (Prophetic) Demonstration that a Transgenic Mouse Harboring a DNA-Encoded Influenza Encrypted RNA Cassette has Increased Resistance to Influenza Infection

The transgenic animal model developed in Example 34 can be infected with influenza and the level and severity of infection can be quantified by measuring body weight loss, observation with clinical scoring, histopathology, and measurement of infectious viral titers within the lungs from samples collected postmortem.

Transgenic animals with germline expression of an antiviral therapeutic influenza encrypted RNA via a DNA-encoded therapeutic encrypted RNA cassette are expected to show increased resistance to influenza infection in comparison to control mice that are modified with a DNA-encoded non-therapeutic encrypted RNA cassette.

Thus, transgenic animals harboring an antiviral influenza encrypted RNA cassette should have substantially reduced disease, significantly lower levels of viral replication, and decreased morbidity/mortality with respect to control animals. In contrast, influenza infection of control animals should lead to typical infection kinetics, disease, and viral loads consistent with normal influenza replication in these mice.

Given that some embodiments of influenza encrypted RNAs can be activated by a diversity of influenza A and B strains, we anticipate that transgenic animals harboring a therapeutic influenza encrypted RNA cassette would be resistant to multiple strains of influenza.

### Example 36: Construction of Parainfluenza Virus Encrypted RNAs

### Parainfluenza Virus Antisense Encrypted RNA Scaffolds

DNA sequences were cloned by standard molecular biology methods as described in Example 1. For parainfluenza virus antisense encrypted RNAs, some L and R flanking nucleotide sequences were obtained by concatenation of nucleotide regions from publicly available human parainfluenza virus 1 (NCBI GenBank ID: KF687313.1) or human parainfluenza virus 3 sequences (Wash/47885/57 strain). All nucleotide numbering in this example is in reference to these nucleotide sequences. The L flank sequence for HPIV1 (antis_5p_HPIV1_FR2011, SEQ ID NO: 181) is comprised of a 157 nt region (nt 1-157) from the 5′ end of the HPIV1 RNA (5′ trailer of L gene end sequence and NS1 gene start). The R flank (antis_3p_HPIV1_FR2011, SEQ ID NO: 184) for HPIV1 is comprised of the terminal 119 nt from the 3′ end of the HPIV1 RNA comprising the untranslated region, gene start, and 3′-Leader sequences. Similarly, the L flank sequence for HPIV3 (antis_5p_HPIV3_Wash, SEQ ID NO: 187) is comprised of a 157 nt region (nt 1-157) from the 5′ end of the HPIV3 RNA (5′ trailer of L gene end sequence and NS1 gene start). The R flank (antis_3p_HPIV3_Wash, SEQ ID NO: 190) for HPIV3 is comprised of the terminal 110 nt from the 3′ end of the HPIV3 RNA comprising the untranslated region, gene start, and 3′-Leader sequences.

The initial HPIV antisense encrypted RNA scaffolds were obtained by combining three independent sequence blocks developed above. For example, ERNA-HPIV1-001-GDura was generated by concatenation of antis_5p_HPIV1_FR2011 (SEQ ID NO: 181), the reverse complement of the coding sequence of Gdura (rcCDS_Gdura, SEQ NO: 301), and antis_3p_HPIV1_FR2011 (SEQ ID NO: 184) in 5′ to 3′ order. Likewise, ERNA-HPIV3-001-Gdura was generated by concatenation of antis_5p_HPIV3_Wash (SEQ ID NO: 187), the reverse complement of the coding sequence of Gdura (rcCDS_Gdura, SEQ NO 301), and antis_3p_HPIV3_Wash (SEQ ID NO: 190) in 5′ to 3′ order. Some examples of additional parainfluenzavirus antisense encrypted RNA scaffolds are listed in Table 2 (compatible with “Rule of 6”).

Introduction of Additional Protein Payloads into Parainfluenza Virus Encrypted RNA Scaffolds

The above HPIV encrypted RNA scaffolds shown with GDura as the polypeptide of interest were used as a basis to similarly encode alternative polypeptides of interest as listed in Table 10, including: EGFP, Rluc8, human IFN-β, human IFN-lambda1, human IFN-lambda3, mouse IFN-β, mouse IFN-lambda2, mouse IFN-lambda3, Syrian hamster IFN-β, or domestic ferret IFN-β. HPIV antisense scaffolds encoding alternative polypeptides of interest were created by replacing rcCDS_Gdura in ERNA-HPIV1-001-Gdura or ERNA-HPIV3-001-Gdura with an antisense CDS selected from Table 10. Following the convention of the previous nomenclature, “ERNA-HPIV1-X-Z” indicates that HPIV1 encrypted RNA Scaffold X encodes polypeptide of interest Z. In the same way, “ERNA-HPIV3-X-Z” indicates that HPIV3 encrypted RNA Scaffold X encodes polypeptide of interest Z.

### DNA-Encoding of HPIV Encrypted RNA for In Vitro Production of RNA

To produce capped HPIV encrypted RNAs or 5′-triphosphorylated HPIV encrypted RNAs, HPIV encrypted scaffolds were cloned into the pAT201 vector described in Example 1. Generated constructs were used as templates for IVT, using either standard unmodified nucleotides (A, C, G, U) or in combination with modified nucleotides, as above in Example 14. Encrypted RNAs were shown to be efficiently activated when using unmodified nucleotides (A, C, G, U) and in combination with various amounts of N1-methylpseudouridine (60-100%). In principle, other nucleotides modifications (e.g. pseudouridine, N6-methyladenosine, 5-methylcytidine, 5-methoxyuridine) can be used in IVT rection in combination with standard unmodified or other nucleotide modifications to produce activatable HPIV encrypted RNA as shown for RSV encrypted RNA.

### Example 33: Construction of Metapneumovirus Encrypted RNAs

Metapneumovirus antisense encrypted RNA scaffolds

DNA sequences were cloned by standard molecular biology methods as described in Example 1. For metapneumovirus virus antisense encrypted RNAs, some L and R flanking nucleotide sequences were obtained by concatenation of nucleotide regions from publicly available human metapneumovirus sequences (e.g., NCBI GenBank ID: AY525843.1). All nucleotide numbering in this example is in reference to this nucleotide sequence. The L flank sequence for HMPV (antis_5p_HMPV_NL, SEQ ID NO: 196) is comprised of a 241 nt region (nt 1-241) from the 5′ end of the HMPV RNA (5′ trailer of L gene end sequence and NS1 gene start). The R flank (antis_3p_HMPV_NL, SEQ ID NO: 201) for HMPV is comprised of the terminal 53 nt from the 3′ end of the HMPV RNA comprising the untranslated region, gene start, and 3′-Leader sequences.

The initial HMPV antisense encrypted RNA scaffolds were obtained by combining three independent sequence blocks developed above. For example, ERNA-HMPV-001-GDura was generated by concatenation of antis_5p_HMPV_NL (SEQ ID NO: 196), the reverse complement of the coding sequence of GDura (rcCDS_GDura, SEQ NO 301), and antis_3p_HMPV_NL (SEQ ID NO: 201) in 5′ to 3′ order.

Introduction of Additional Protein Payloads into Metapneumovirus Encrypted RNA Scaffolds

The above HMPV encrypted RNA scaffolds shown with GDura as the polypeptide of interest were used as a basis to similarly encode alternative polypeptides of interest as listed in Table 10, including: EGFP, Rluc8, human IFN-β, human IFN-lambda1, human IFN-lambda3, mouse IFN-β, mouse IFN-lambda2, mouse IFN-lambda3, Syrian hamster IFN-β, or domestic ferret IFN-β. HMPV antisense scaffolds encoding alternative polypeptides of interest were created by replacing rcCDS_GDura in ERNA-HMPV-001-GDura with an antisense CDS selected from Table 10. Following the convention of the previous nomenclature, “ERNA-HMPV-X-Z” indicates that HMPV encrypted RNA Scaffold X encodes polypeptide of interest Z. Some examples of additional metapneumovirus antisense encrypted RNA scaffolds are listed in Table 2 (compatible with “Rule of 6”).

### DNA-Encoding of HMPV Encrypted RNA for In Vitro Production of RNA

To produce capped HMPV encrypted RNA or 5′-triphosphorylated HMPV encrypted RNA, HMPV encrypted scaffolds were cloned into the pAT201 vector described in Example 1. Generated constructs were used as templates for IVT, using either standard unmodified nucleotides (A, C, G, U) or in combination with modified nucleotides, as above in Example 14. Encrypted RNAs were shown to be efficiently activated when using unmodified nucleotides (A, C, G, U) and in combination with various amounts of N1-methylpseudouridine (60-100%). In principle, other nucleotides modifications (e.g. pseudouridine, N6-methyladenosine, 5-methylcytidine, 5-methoxyuridine) can be used in IVT rection in combination with standard unmodified or other nucleotide modifications to produce activatable HMPV encrypted RNA as shown for RSV encrypted RNA (see FIG. 66).

### Example 34: Construction of Henipavirus Encrypted RNAs

### Henipavirus Antisense Encrypted RNA Scaffolds

DNA sequences were cloned by standard molecular biology methods as described in Example 1. For henipavirus antisense encrypted RNAs, some L and R flanking nucleotide sequences were obtained by concatenation of nucleotide regions from publicly available Nipah virus (NCBI GenBank ID: AY029767) or Hendra virus (NCBI GenBank ID: AF017149) sequences. All nucleotide numbering in this example is in reference to these nucleotide sequences. The L flank sequence for NiV (antis_5p_NiV_UMMC1, SEQ ID NO: 204) is comprised of a 100 nt region (nt 1-100) from the 5′ end of the NiV genomic RNA (viral RNA) (5′ trailer of L gene end sequence and NS1 gene start). The R flank (antis_3p_NiV_UMMC1, SEQ ID NO: 206) for NiV is comprised of the terminal 112 nt from the 3′ end of the NiV genomic RNA comprising the untranslated region, gene start, and 3′-Leader sequences. Similarly, the L flank sequence for HeV (antis_5p_HeV_AUS, SEQ ID NO: 209) is comprised of a 10 nt region (nt 1-100) from the 5′ end of the Hendra virus genomic RNA (viral RNA) (5′ trailer of L gene end sequence and NS1 gene start). The R flank (antis_3p_HeV_AUS, SEQ ID NO: 211) for HeV is comprised of the terminal 112 nt from the 3′ end of the HeV RNA comprising the untranslated region, gene start, and 3′-Leader sequences.

The initial henipavirus antisense encrypted RNA scaffolds were obtained by combining three independent sequence blocks developed above. For example, ERNA-NiV-001-GDura was generated by concatenation of antis_5p_NiV_UMMC1 (SEQ ID NO: 204), the reverse complement of the coding sequence of GDura (rcCDS_GDura, SEQ NO: 301), and antis_3p_NiV_UMMC1 (SEQ ID NO: 206) in 5′ to 3′ order. Likewise, ERNA-HeV-001-GDura was generated by concatenation of antis_5p_HeV_AUS (SEQ ID NO: 209), the reverse complement of the coding sequence of GDura (rcCDS_GDura, SEQ NO 301), and antis_3p_HeV_AUS (SEQ ID NO: 211) in 5′ to 3′ order.

Introduction of Additional Protein Payloads into Henipavirus Encrypted RNA Scaffolds

The above henipavirus encrypted RNA scaffolds shown with GDura as the polypeptide of interest were used as a basis to similarly encode alternative polypeptides of interest as listed in Table 5 or Table 10, including: EGFP, Rluc8, human IFN-β, human IFN-lambda1, human IFN-lambda3, mouse IFN-β, mouse IFN-lambda2, mouse IFN-lambda3, Syrian hamster IFN-β, or domestic ferret IFN-β. Henipavirus antisense scaffolds encoding alternative polypeptides of interest were created by replacing rcCDS_GDura in ERNA-NiV-001-GDura or ERNA-HeV-001-GDura with an antisense CDS selected from Table 5 or Table 10. Following the convention of the previous nomenclature, “ERNA-NiV-X-Z” indicates that NiV encrypted RNA Scaffold X encodes polypeptide of interest Z. In the same way, “ERNA-HeV-X-Z” indicates that HeV encrypted RNA Scaffold X encodes polypeptide of interest Z. Some examples of additional henipavirus antisense encrypted RNA scaffolds are listed in Table 2 (compatible with “Rule of 6”).

### DNA-Encoding of Henipavirus Encrypted RNA for In Vitro Production of RNA

To produce capped henipavirus encrypted RNA or 5′-triphosphorylated henipavirus encrypted RNA, henipavirus encrypted scaffolds were cloned into the pAT201 vector described in Example 1. Generated constructs were used as templates for IVT, using either standard unmodified nucleotides (A, C, G, U) or in combination with modified nucleotides, as above in Example 14. Encrypted RNAs were shown to be efficiently activated when using unmodified nucleotides (A, C, G, U) and in combination with various amounts of N1-methylpseudouridine (60-100%). In principle, other nucleotides modifications (e.g. pseudouridine, N6-methyladenosine, 5-methylcytidine, 5-methoxyuridine) can be used in IVT rection in combination with standard unmodified or other nucleotide modifications to produce activatable henipavirus encrypted RNA as shown for RSV encrypted RNA (see FIG. 66).

### Example 35: Construction of Hepadnavirus Encrypted RNAs

### Hepadnavirus Antisense Encrypted RNA Scaffolds

DNA sequences were cloned by standard molecular biology methods as described in Example 1. For hepadnavirus antisense encrypted RNAs, some L and R flanking nucleotide sequences were obtained by concatenation of nucleotide regions from publicly available HBV sequences (e.g., strain ayw; NCBI GenBank ID: FZ421397) with additional nucleotides sequences interposed. All nucleotide numbering is in reference to these nucleotide sequences. The L flank sequence for HBV (antis_5p_HBV_01, SEQ ID NO: 222) is comprised of a subsequence of the 5′ end portion of HBV pre-genomic RNA and an additional reverse complementary coding sequence for an RFP. The R flank (antis_3p_HBV_01, SEQ ID NO: 225) for HBV is comprised of nucleotides from the 3′ end of HBV pre-genomic RNA. An alternative L flank sequence was developed (antis_5p_HBV_02, SEQ ID NO: 223) which omitted the RFP present in antis_5p_HBV__01 (SEQ ID NO: 222).

The initial hepadnavirus antisense encrypted RNA scaffolds were obtained by combining three independent sequence blocks developed above. For example, ERNA-HBV-001-GDura was generated by concatenation of antis_5p_HBV_01 (SEQ ID NO: 222), the reverse complement of the coding sequence of GDura (rcCDS_GDura, SEQ NO: 301), and antis_3p_HBV_01 (SEQ ID NO: 225) in 5′ to 3′ order. Likewise, ERNA-HBV-002-GDura was generated similarly to ERNA-HBV-001-GDura, except antis_5p_HBV_02 (SEQ ID NO: 223) was substituted for antis_5p_HBV_01 (SEQ ID NO: 222). Additional hepadnavirus antisense encrypted RNA scaffolds were prepared using the above scheme with novel L and R sequences as detailed above in Table 2 (e.g., ERNA-HBV-003, ERNA-HBV-004).

Introduction of Additional Protein Payloads into Hepadnavirus Encrypted RNA Scaffolds

The above hepadnavirus encrypted RNA scaffolds shown with GDura as the polypeptide of interest were used as a basis to similarly encode alternative polypeptides of interest as listed in Table 5 or Table 10, including: EGFP, Rluc8, human IFN-β, human IFN-lambda1, human IFN-lambda3, mouse IFN-β, mouse IFN-lambda2, mouse IFN-lambda3, Syrian hamster IFN-β, or domestic ferret IFN-β. Hepadnavirus antisense encrypted RNA scaffolds encoding alternative polypeptides of interest were created by replacing rcCDS_GDura in ERNA-HBV-001-GDura with an antisense CDS selected from Table 5 or Table 10. Following the convention of the previous nomenclature, “ERNA-HBV-X-Z” indicates that HBV encrypted RNA Scaffold X encodes polypeptide of interest Z. In the same way, “ERNA-HBV-X-Z” indicates that HBV encrypted RNA Scaffold X encodes polypeptide of interest Z.

### Hepadnavirus Sense Encrypted RNA Scaffolds

HBV sense encrypted RNAs scaffolds were also prepared. To form sense hepadnavirus encrypted RNA scaffolds, three new L blocks were generated: sense_5p_HBV_21 (SEQ ID NO: 212), sense_5p_31 (SEQ ID NO: 213), and sense_5p_41 (SEQ ID NO: 214). Paired R blocks were also developed: sense_3p_HBV_21 (SEQ ID NO: 217))), sense_3p_HBV_31 (SEQ ID NO: 218), and sense_3p_HBV_41 (SEQ ID NO: 219). Importantly, the L sequence blocks incorporate a “2A” ribosomal skip sequence from Thosea asigna virus (RS_T2A, SEQ ID NO: 330) at the 5′ end of each sequence. Use of the “2A” site may be improved when the when the coding sequence following the ribosomal skip sequence (SEQ ID NO: 330) lacks the initial start codon.

The initial sense HBV encrypted RNA scaffolds were obtained by combining three independent sequence blocks described above. For example, ERNA-HBV-101-GDura was generated by concatenating sense_5p_HBV_21 (SEQ ID NO: 212), CDS_GDura (SEQ ID NO: 273), and sense_3p_HBV_41 (SEQ ID NO: 219) in 5′ to 3′ order. Similarly, ERNA-HBV-102-GDura was generated by concatenation sense_5p_HBV_41 (SEQ ID NO: 214), CDS_GDura (SEQ ID NO: 273), and sense_3p_HBV_41 (SEQ ID NO: 219) in 5′ to 3′ order. Two similar scaffolds were also prepared in which the CDS lacked a conventional start codon by deletion of the first 3 nucleotides of the CDS (typically ATG). These sequences are referred to as nsCDS, no start (codon) coding sequences and abbreviate their formation by prepending “ns” to the sequence name. As an example, CDS_GDura (SEQ ID NO: 273) is 558 nt in length and begins with “atg” while nsCDS_GDura is 555 nt in length (558-3) (and begins with “gga”).

Introduction of Additional Protein Payloads into HBV Encrypted RNA Scaffolds

The above HBV encrypted RNA scaffolds shown with GDura as the polypeptide of interest were used as a basis to similarly encode alternative polypeptides of interest as listed in Table 5 or Table 10, including: EGFP, Rluc8, human IFN-β, human IFN-lambda1, human IFN-lambda3, mouse IFN-β, mouse IFN-lambda2, mouse IFN-lambda3, Syrian hamster IFN-β, or domestic ferret IFN-β. HBV antisense scaffolds encoding alternative polypeptides of interest were created by replacing rcCDS_GDura in ERNA-HBV-001-GDura,—with an antisense CDS selected from Table 5. HBV sense encrypted RNA scaffolds encoding alternative polypeptides of interest were created by replacing CDS_GDura in ERNA-HBV-101, -102-GDura, with a sense CDS selected from Table 5. Similarly, HBV sense encrypted RNA scaffolds encoding alternative polypeptides of interest were created by replacing nsCDS_GDura in ERNA-HBV-101, -102-GDura, with a sense nsCDS by selecting an CDS from Table 5 and removing the first 3 nucleotides (the start codon). Following the convention of the previous nomenclature, “ERNA-HBV-X-Z” indicates that HBV encrypted RNA Scaffold X encodes polypeptide of interest Z.

### Activation of an HBV Encrypted RNA by a Translation Activator Encoded by HBV

Huh7-NTCP cells (Huh7 cells which overexpress human NTCP, the entry receptor for HBV) were transduced with a lentivirus encoding hepadnavirus pregenomic RNA (HBV pgRNA) and selected using puromycin at a final concentration of 10 μg/ml. After puromycin selection cells were expanded to 12-well plates for transfection with a hepadnavirus encrypted RNA. Huh7-NTCP and Huh7-NTCP HBV pgRNA were plated on 12-well plates at 9E04 cells/well. About 20 hours later, both cell populations were transfected with 57 ng/cm² LMAX-LNP formulated ERNA-HBV-105-GDura. About 20 hours after LMAX-LNP treatment, cell media was collected, cells were washed 3 times with DPBS, and fresh medium was added into wells. After medium replacement, 100 mcL of supernatant was collected as baseline or time zero. One hundred mcL of supernatant was collected at 2-, 4-, and 6-days post-transfection. All collected supernatants were clarified from the cell debris by centrifugation. Ten mcL of collected media was transferred to 96-well black plate and bioluminescent signal of expressed GDura reporter were measured, as previously described.

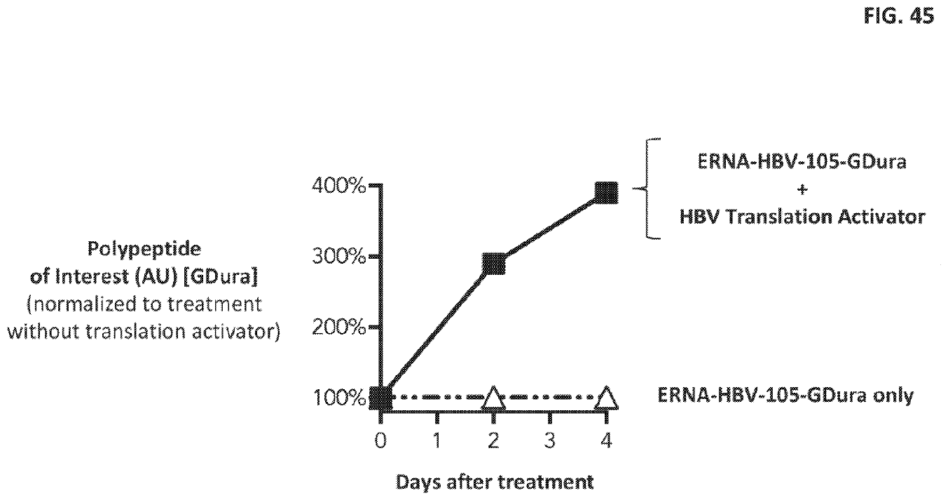

FIG. 45 shows the increase in GDura expression in Huh7-NTCP HBV pgRNA cells over Huh7-NTCP cells, when both cell populations were treated with same hepadnavirus encrypted RNA. Thus, a hepadnavirus encrypted RNA (ERNA-HBV-105-GDura) can be substantially activated by providing a translation activator, comprising the core protein of HBV (e.g. via pgRNA expression within Huh-7 NTCP HBV pgRNA cells).

### Example 36: Stabilization of Encrypted RNA by Complexation with RNA-Binding Proteins

Enhanced Lifetime of RSV Encrypted RNAs by Viral Infection of Treated Cells Days after Encrypted RNA Treatment

Complexation of encrypted RNAs with RNA-binding proteins, such as nucleocapsid proteins, can stabilize encrypted RNAs by diverse mechanisms, including: slowing intrinsic metal-catalyzed hydrolysis of RNA, inhibiting nuclease activity on RNA by steric hindrance, or by preventing access of host innate immune sensors to potential immunostimulatory motifs present on some RNAs (e.g., 5′-triphosphates).

Using the study design taught in Example 25, the stabilizing function of RNA-binding proteins (“RBPs”) on an RSV antisense encrypted RNA (such as ERNA-RSV-005-GDura or ERNA-RSV-001-GDura) was tested. The study was performed as described in Example 25, except that cells were additionally treated by providing 0, 1, or 2 mRNAs encoding RNA-binding proteins: (Treatment 1) no additional mRNA; (Treatment 2) an mRNA encoding for RSV N (polypeptide SEQ ID NO: 400); (Treatment 3) an mRNA encoding for RSV P (polypeptide SEQ ID NO: 401); (Treatment 4) both an mRNA encoding for RSV N and an mRNA encoding for RSV P.

The “functional persistence” of an RSV encrypted RNA (ERNA-RSV-005-GDura) was substantially increased when cells were co-transfected with mRNAs encoding RSV N (Treatment 2) or RSV N and P proteins (Treatment 4). Functional persistence in this example means the ability of an encrypted RNA to both persist in cells and remain capable of activation by a translation activator. Cultures treated with Treatment 2 (RSV N) or Treatment 4 (RSV N+RSV P) demonstrated substantially increased functional persistence as the interval between encrypted RNA treatment and RSV infection was extended to 5 days or more. In contrast, there was no substantial effect on the functional persistence of the RSV encrypted RNA in Treatment 1 (no additional mRNA) or Treatment 3 (mRNA encoding RSV P).

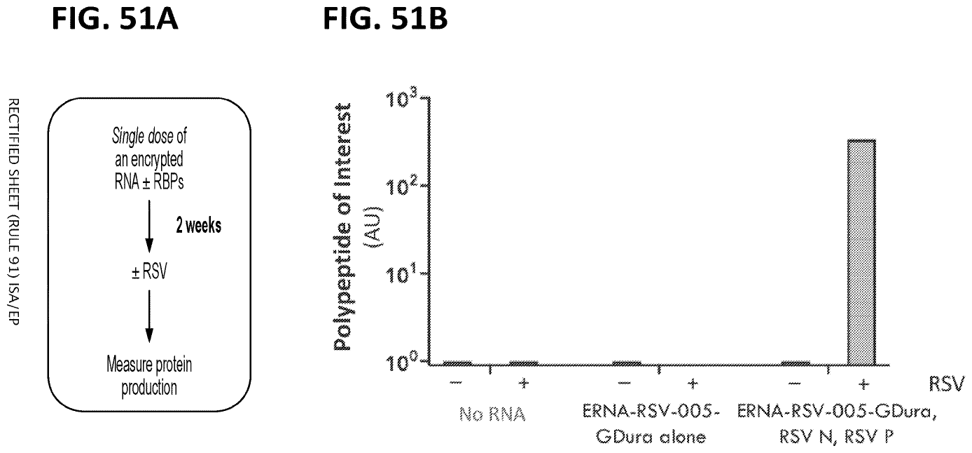

FIG. 51A shows a simplified schematic of an experiment to test the functional persistence of an RSV encrypted RNA.

FIG. 51B shows the result of one such experiment in cells treated with ERNA-RSV-005-GDura on Day 0. RSV infection 14 days later resulted in activation levels >100× above background when cells were co-transfected with Treatment 4 (RSV N and RSV P) on Day 0. Notably, over the course of the 14 day study, production of the polypeptide of interest remained below the level of detection in the absence of RSV (i.e. the encrypted RNA persisted but was not activated above background levels absent a translation activator). Further, the RSV encrypted RNA was not substantially activated by RSV infection on Day 14 if treated cells did not receive N and P complementation (Treatment 1) on Day 0.

More broadly, FIGS. 51A-51B demonstrate that the functional persistence of an encrypted RNA in treated cells can be substantially increased by complexing the encrypted RNA with RNA-binding proteins, such as nucleoproteins.

Enhanced Lifetime of Sarbecovirus Encrypted RNAs by Viral Infection of Treated Cells Days after Encrypted RNA Treatment

We also tested the stabilizing function of RNA-binding proteins on sarbecovirus-encrypted RNA. At 16 hours before infection, Vero-E6-hACE2+ORF3a/E cells were plated in 48-well plates at a density of 4×10⁴ cells/well in D10 supplemented to a final concentration of 0.2 μg/mL doxycycline (to induce expression of ORF3a & E) and 10 μg/mL puromycin (to maintain selection for the inducible ORF3a/E cassette). At 3 h prior to infection, cells were pretreated with 20 ng each of an LNP-formulated mRNA encoding the SARS-CoV-2 nucleocapsid (N) protein (polypeptide SEQ ID NO: 404) and an LNP-formulated mRNA encoding the N-terminal fragment (amino acids 1-206) of the SARS-COV-2 Nsp3 protein (polypeptide SEQ ID NO: 405). An LNP-formulated mRNA encoding a GFP was used as a control. An additional, non-limiting example would be providing SARS-COV-1 N (polypeptide SEQ ID NO: 402) or SARS-CoV-1 Orf3a (polypeptide SEQ ID NO: 403).

Cells were mock-infected (MOI 0) or infected at an MOI 1 with SARS2-GL at MOI 1 for 1 h by inoculation with SARS2-GL for 1 h at 37° C. in a humidified 5% CO₂ incubator. Immediately after the 1 h infection, cells were treated with 10 ng of an LNP formulated encrypted RNA (ERNA-SARS2-001-GDura). Over the subsequent three days, culture media was collected daily (24 hpi, 48 hpi, 72 hpi) and assayed for production of the GDura polypeptide of interest.

Cultures pretreated with SARS-N or both SARS-N and Nsp3a mRNAs demonstrated higher activation in response to viral infection compared to the cultures pretreated with Nsp3a mRNA only or control EGFP mRNA. For example, cultures pretreated with both SARS-N and Nsp3a mRNAs demonstrated up to three-fold increase activation at 0-48 hr interval between encrypted RNA treatment and SARS infection. Additional analysis of SARS-N expression by Western blot demonstrated that co-transfection with Nsp3a increases SARS-N stability in Vero cells (data not shown).

### Example 37: Activation of Encrypted RNAs by Providing Isolated mRNAs that can be Translated in Cells to Comprise a Target-Specific Translation Activator

Example 3 and Example 23 demonstrate that encrypted RNAs can be activated by a target-specific translation activator when the translation activator is provided in trans by co-transfected expression plasmids encoding the translation activator. This Example demonstrates that encrypted RNAs can also be activated by providing a target-specific translation activator as a set of one or more isolated mRNAs, which together comprise a target-specific translation activator.

Pools of up to four purified messenger RNAs encoding for RSV proteins L, M2-1 N, or P, were prepared using methods known to one skilled in the art. ERNA-RSV-005-GDura was prepared as described in Example 23.

Three mRNA pools (comprised of mRNA components selected from L, M2-1, N, or P) were prepared: (i) “mRNA pool L3” was comprised of a mixture of L mRNA, M2-1 mRNA, N mRNA, and P mRNA at a mass ratio of 3:1:1:1 (L:M2-1:N:P); (ii) “mRNA pool N/P” was comprised of N mRNA and P mRNA at a mass ratio of 1:1; and (iii) “mRNA pool L/M2-1” was comprised of L mRNA and M2-1 mRNA at a mass ratio of 3:1.

In Method A, human AD293 cells in 48-well plates were treated simultaneously by one of the following: (Treatment 1) no RNA; (Treatment 2) ~80 ng of ERNA-RSV-005-GDura; (Treatment 3) ~120 ng of “mRNA Pool L3”; or (Treatment 4) ~80 ng of ERNA-RSV-005-GDura and ~120 ng of “mRNA Pool L3”. The amount of the translated polypeptide of interest (secreted GDura luciferase) was quantified at 24 and 48 hours post-treatment as described above. In this Example, if the level of GDura increased more than 10× relative to untransfected cells, then the polypeptide of interest (GDura) was said to have increased translation.

Only negligible, background levels of translation of the polypeptide of interest were observed in untreated cells (Treatment 1), in cells receiving an encrypted RNA alone (Treatment 2), or in cells only receiving mRNAs encoding the translation activator (Treatment 3). In contrast, the polypeptide of interest (GDura) had increased translation when cells were treated with both an encrypted RNA and a target-specific translation activator of the encrypted RNA (Treatment 4), reaching ~1,000× above background levels at 48 hours after treatment.

TABLE 16
Sequential Treatment with Components of a Translation Activator

| | | Subsequent Treatment | Activation |
|---|---|---|---|
| Treatment | First Treatment (0 h) | (24 h) | at 96 h |
| 21 | None | None | ~1× |
| | | | (background) |
| 22 | incomplete translation | incomplete translation | ~1× |
| | activator | activator | (background) |
| | (mRNA Pool N/P) | (mRNA Pool L/M2-1) | |
| 23 | Encrypted RNA | None | ~1× |
| | (ERNA-RSV-005- | | (background) |
| | GDura) + | | |
| | incomplete | | |
| | translation activator | | |
| | (mRNA Pool N/P) | | |
| 24 | Encrypted RNA | incomplete translation | ~1× |
| | (ERNA-RSV-005- | activator | (background) |
| | GDura) | (mRNA Pool L/M2-1) | |
| 25 | Encrypted RNA | incomplete translation | ~1,000× |
| | (ERNA-RSV-005- | activator | |
| | GDura) + | (mRNA Pool L/M2-1) | |
| | complete translation | | |
| | activator | | |
| | (mRNA Pool N/P) | | |

In Method B, human AD293 cells in 48-well plates were treated with RNA sequentially, first at 0 h and subsequently 24 hours later (24 h) with different RNAs according to the schedule listed in Table 16. The amount of the translated polypeptide of interest (secreted GDura luciferase) was quantified at 96 h post-treatment as described above. In this Example, if the level of GDura increased more than 10× relative to untransfected cells, then the polypeptide of interest (GDura) was said to have increased translation. Only negligible, background levels of translation of the polypeptide of interest were observed in untreated cells (Treatment 21) or in cells receiving an encrypted RNA but an incomplete target-specific translation activator (Treatment 23 or Treatment 24), as shown in Table 16. Likewise, cells receiving a complete translation activator but no encrypted RNA (Treatment 22) had only background translation of the polypeptide of interest (GDura).

In contrast, the polypeptide of interest (GDura) had increased translation when cells were treated with both an encrypted RNA and a target-specific translation activator of the encrypted RNA (Treatment 25), even if the precursor components of a translation activator were applied independently and sequentially across 24 hours as isolated mRNAs. In another way, these results further demonstrate that target-specific translation activators can be constituted in cells to activate encrypted RNAs by providing individual components of target-specific translation activators by independent methods (e.g. as plasmids, mRNAs, or via viral infections) and at distinct times.

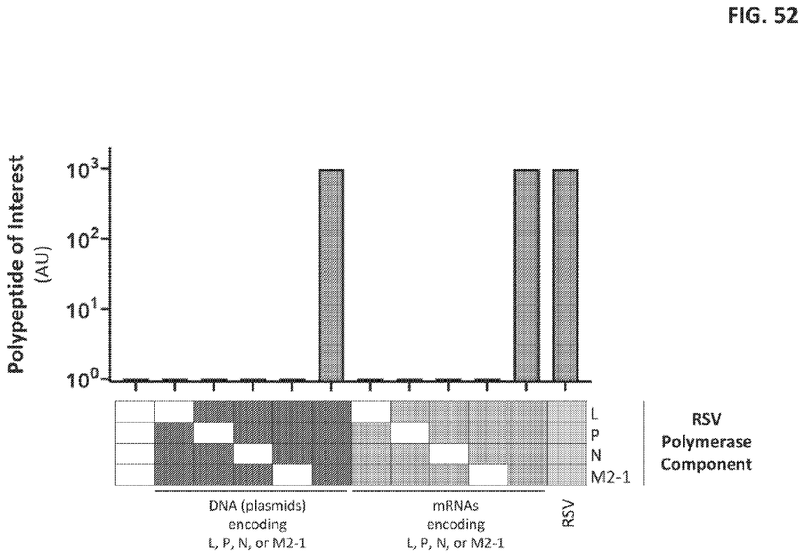

FIG. 52 shows that, in some embodiments, LNP-encapsulated ERNA-RSV-005-GDura can be substantially activated (in the absence of viral infection) via co-transfection with plasmids or mRNAs encoding RSV proteins L, N, M2-1, and P (together a translation activator of the RSV encrypted RNA). The approach for plasmid co-transfection followed the approach used in Example 23 (FIG. 42), and the approach for co-transfection with mRNAs followed the approach in Method B of this example. In both cases, activation of the encrypted RNA was increased by ~3 logs within 48 hours, as quantified by the luciferase assay described above, when each element encoding L, N, M2-1, and P was co-transfected to the cells.

### Example 38: Production of Immunomodulatory or Anti-Neoplastic Proteins with Sencrypted RNA

Encrypted RNA scaffolds can be used to encode a variety of coding sequences, including those that encode proteins with known immunomodulatory or anti-neoplastic properties. Non-limiting examples include caspase 3 (RevCap3), interleukin-1 receptor antagonist (IL1RN), tumor necrosis factor (TNF), interleukin 2 (IL-2), interleukin-2 variants with altered activity (e.g. superkines), or interleukin-12 (IL-12). The encoded polypeptide of interest can have immunomodulatory or anti-neoplastic action either directly on the treated cell or indirectly, for example through paracrine secretion.

AD293 cells or A549 cells were treated using Method B in Example 37, where an RSV encrypted RNA encoding the human IL-2 precursor (ERNA-RSV-005-IL2) was substituted for ERNA-RSV-005-GDura. A parallel experiment was conducted contemporaneously using ERNA-RSV-005-GDura as the encrypted RNA to test the specificity of IL-2 secretion to the encoded protein payload. At 96 h, the levels of secreted IL-2 in the cell media were quantified by ELISA (Human IL-2 ELISA Kit, Protein Tech; Cat No: KE00017).

Cultures treated with the IL-2 encoding encrypted RNA (ENA-RSV-005-hu_IL_2) and a complete target-specific translation activator (per Treatment 25 in Table 16) had IL-2 levels of ~40 μg/mL (~0.65 IU/mL) at 96 h. In contrast, all other treatment schemes (except for Treatment 25) when the encrypted RNA was ERNA-RSV-005-IL2 had IL-2 levels below the limit of detection (<3.6 μg/mL). Importantly, an encrypted RNA encoding IL-2 (ERNA-RSV-005-IL2) was required for IL-2 secretion, as all Treatments (21-25) with ERNA-RSV-005-GDura had IL-2 levels below the limit of detection (<3.6 μg/mL) at 96 h.

To test the production of interleukin-1 receptor antagonist (IL-1RN or IL-1RA) from an encrypted RNA encoding CDS_ILIRN, AD293 cells were treated using Method A in Example 37. An RSV encrypted RNA encoding the human IL-1RN precursor (ERNA-RSV-005-IL1RN) substituted for ERNA-RSV-005-GDura. A parallel experiment was conducted contemporaneously using ERNA-RSV-005-GDura as the encrypted RNA to test the specificity of IL-1RN secretion to the encoded protein payload. At 72 h, the levels of secreted IL-2 in the cell media were quantified by ELISA.

Cultures treated with the IL-1RN encoding encrypted RNA (ENA-RSV-005-IL1RN) and a complete target-specific translation activator (per Treatment 4) had IL-1RN levels of ~3000 μg/mL at 72 h. In contrast, all other treatment schemes (those except for Treatment 24) when the encrypted RNA was ERNA-RSV-005-IL1RN had undetectable levels of IL-1RN (below the limit of detection of 46 μg/mL).

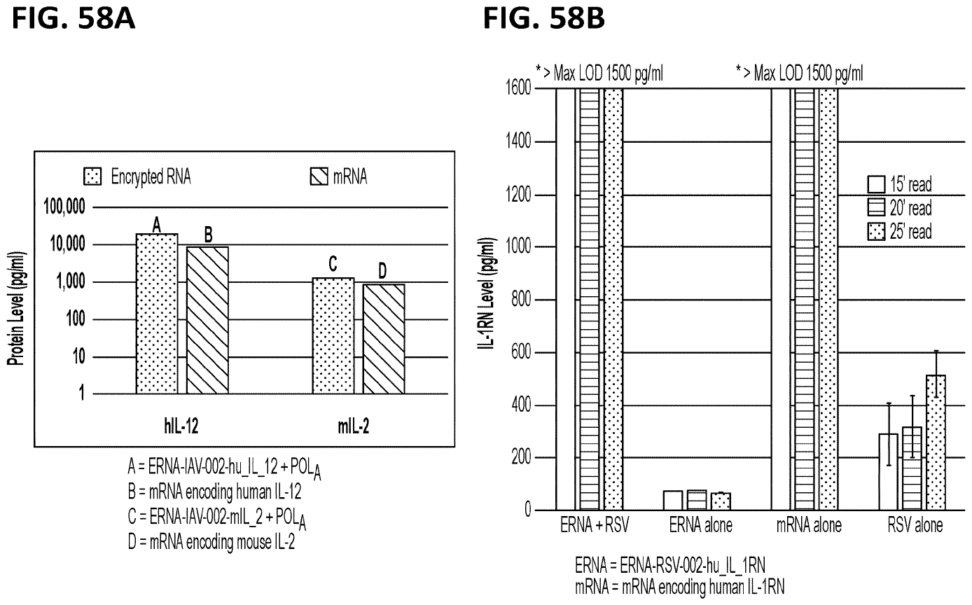

FIGS. 58A-58B show the plug-and-play capability of both influenza and RSV encrypted RNA scaffolds to encode immunomodulatory proteins as polypeptides of interest.

FIG. 58A shows an influenza encrypted RNA scaffold (ERNA-IAV-002-hu_IL_2) encoding human IL-12 (ERNA-IAV-002-hu_IL_12) or mouse IL-2 (ERNA-IAV-002-mIL_2) as the polypeptide of interest.

In the case of human IL-12, an LNP-formulated ERNA-IAV-002-hu_IL_12 encrypted RNA was used to treat human AD293 cells, which were co-transfected with LNPs encapsulating mRNAs encoding influenza (A/PR8) PB1, PB2, PA and NP proteins (i.e. PolA). The level of activation of the polypeptide of interest was quantified by an ELISA specific for human IL-12, using an assay analogous to that described above for human IL-2. Notably, the translated level of the human IL-12 polypeptide of interest by the encrypted RNA in the presence of Pola was higher than the level of translation from a (constitutively active) mRNA encoding human IL-12.

Similarly, an LNP-encapsulated ERNA-IAV-002-m_IL_2 encrypted RNA was used to treat mouse B16-F10 cells, which were co-transfected with LNPs encapsulating mRNAs encoding influenza (A/PR8) PB1, PB2, PA and NP proteins (i.e. PolA). The level of activation of the polypeptide of interest was quantified by an ELISA specific for mouse IL-2, using an assay analogous to that described above for human IL-2. Notably, the translated level of the mouse IL-2 polypeptide of interest by the encrypted RNA in the presence of POLA was higher than the level of translation from a (constitutively active) mRNA encoding mouse IL-2.

Taken together, FIG. 58A shows that an encrypted RNA can be used to encode distinct immunomodulatory proteins for use in human or animal species. FIG. 58A further demonstrates that—by delivering an encrypted RNA encoding these immunomodulatory proteins as polypeptides of interest and a polynucleotide sequence encoding a translation activator of the encrypted RNA—the polypeptides of interest can be translated at high levels outside of viral infections.

Further, because IL-2 and IL-12 are known immunomodulatory proteins that are commonly used as anti-neoplastic therapeutics, a clear use of an encrypted RNA encoding these or other immunomodulatory therapeutics as polypeptides of interest (co-delivered with a polynucleotide sequence encoding a translation activator) is to treat cancers.

A clear advantage of co-delivering an encrypted RNA and nucleic acids that encode a translation activator of the encrypted RNA is that it can enable localized protein translation in vivo (i.e. reduced off-target toxicity). This is because, in such treatments, one way for the encrypted RNA to translate an encoded protein is if it is found in cells that are also treated with a polynucleotide encoding a complete translation activator (i.e. the encrypted RNA approach is essentially an AND gate that requires multiple molecules to probabilistically treat the same cell for an mRNA encoding the polypeptide of interest to be synthesized).

The probability of having multiple polynucleotides in the same cell will decrease approximately exponentially as the distance is increased from the site of treatment (e.g. from a local administration into a tumor). In contrast, if an mRNA encoding an immunomodulatory protein is used as the treatment, the mRNA would only require a single molecule to enter a cell for protein translation to occur. Thus, with an mRNA treatment, there is expected to be more protein translation in tissues more distant from the site of treatment (e.g. outside of a locally treated tumor, in what may be non-cancerous cells that one may aim to avoid treating to increase the therapeutic index).

FIG. 58B shows that an RSV encrypted RNA can analogously encode immunomodulators as polypeptides of interest. In this case, cells were treated with ERNA-RSV-005-IL1_RN encoding IL1-RA (an anti-inflammatory cytokine, which is a known treatment for human autoimmune conditions) as the polypeptide of interest. The translation activator was provided via RSV infection. As was done earlier in this example, IL-1RN production was measured via ELISA. As a positive control, parallel cells were treated with an mRNA encoding IL1-RN in the presence or absence of RSV, and similarly assayed via ELISA. Finally, another set of cells was infected with RSV alone, but not treated with an additional RNA.

FIG. 58B again shows that, in the presence of a translation activator (this time provided by viral infection), an encrypted RNA can translate a therapeutic protein to levels approximately as high as an mRNA constrictively producing that protein. Further, the encrypted RNA produced approximately background levels of the polypeptide of interest in the absence of RSV infection. Finally, RSV alone was not responsible for high levels of IL-1RA production; cells infected with RSV alone and not treated with, for example, the encrypted RNA produced substantially reduced levels of the IL-1RN in comparison to the cells infected with RSV and treated with the encrypted RNA.

### Example 39: Production of Multiple Proteins with Distinct Effects from a Single Encrypted RNA

HEp-2, HEK-Blue, BEAS2B, or A549 cells were treated with a bigenic RSV antisense encrypted RNAs (ERNA-RSV-005-GDura-I1-IFNB or ERNA-RSV-005-IFNB-11-Gdura, the order of rcCDS being inverted) or a monogenic RSV antisense encrypted RNA (ERNA-RSV-005-GDura) and activation measured in the presence or absence of a target-specific translation activator provided by RSV infection (MOI=1), as described in Example 23. For simplicity, only the level of the GDura polypeptide of interest was measured in each experiment.

Only background levels of GDura translation were measured in cell populations that lacked either an encrypted RNA or a target-specific translation activator, specifically populations: (i) treated with an encrypted RNA but not infected with RSV or (ii) infected with RSV but not treated with an encrypted RNA. In contrast, populations which received both a GDura-encoding encrypted RNA and were RSV-infected showed levels 100-10,000× higher than background. The highest levels of GDura translation were observed from ERNA-RSV-005-GDura (~10,000× above background). Significant activation was observed in both bigenic scaffolds: ERNA-RSV-005-GDura-I1-IFNB had translated GDura levels ~1000× above background and ERNA-RSV-005-IFNB-11-GDura had levels ~100× above background. Thus, bigenic encrypted RNA scaffolds can adequately express protein from coding sequences positioned at either locus in the bigenic scaffold in the presence of a target-specific translation activator.

### Example 40: Treatment with Multiple Encrypted RNAs Encoding Unique Payloads but Sharing a Unique Translation Activator

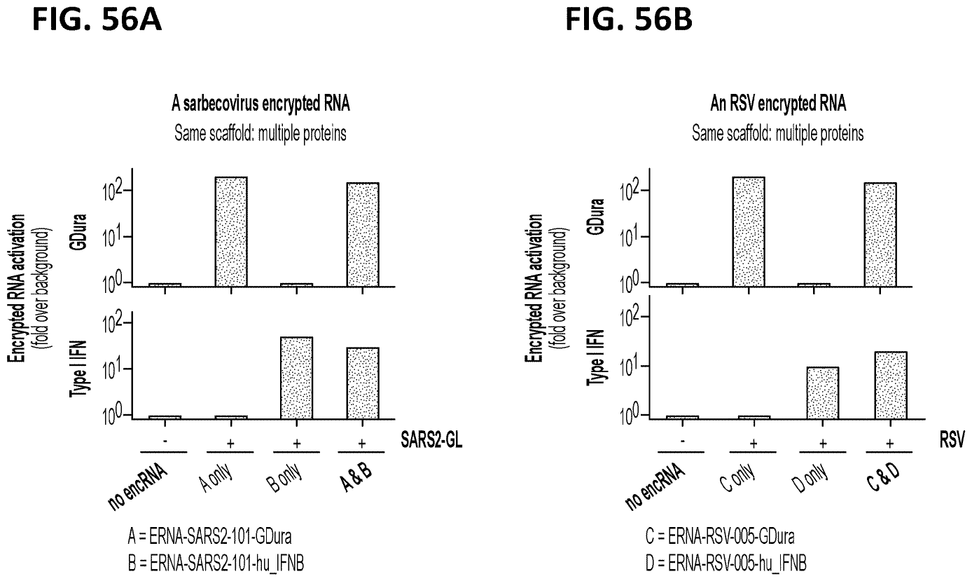

In FIG. 56B, HEp-2 cells were treated using the same methods of Example 22, but were treated with an RSV antisense encrypted RNA encoding GDura (ERNA-RSV-005-GDura), an RSV encrypted RNA encoding mouse IFNB (ERNA-RSV-005-m_IFNB) or both encrypted RNAs (ERNA-RSV-005-GDura+ERNA-RSV-005-m_IFNB). Levels of translated polypeptide of interest (either GDura or mouse IFNB) were quantified at 72 hours after cultures were treated with encrypted RNAs and infected (or not-infected) with RSV at an MOI of 0.1

In the absence of RSV infection or in the absence of encrypted RNA treatment, only negligible, background levels of mouse IFNB or GDura were detected. In cells singly-treated with ERNA-RSV-005-GDura and infected with RSV, levels of secreted luciferase (GDura) of about 100× above background were observed. In cells, singly-treated with ERNA-RSV-005-m_IFNB and infected with RSV, levels of m_IFNB or about 15× above background were observed. With cells treated with both ERNA-RSV-005-m_IFNB and ERNA-RSV-005-GDura, levels of GDura reached ~100× above background and levels of m_IFNB reached >15× above background, approximately the same levels that were observed in cells treated with only one encrypted RNA.

In FIG. 56A, an equivalent experiment was performed using sarbecovirus encrypted RNAs (ERNA-SARS2-101-GDura, ERNA-SARS2-101-m_IFNB) and SARS2-GL infection using the methods of Example 19. In the absence of SARS2-GL infection or in the absence of encrypted RNA treatment, only negligible, background levels of mouse IFNB or GDura were detected. In cells singly-treated with ERNA-SARS2-005-GDura and infected with SARS2-GL, levels of secreted luciferase (GDura) of about 100× above background were observed. In cells, singly-treated with ERNA-SARS2-101-m_IFNB and infected with SARS2-GL, levels of m_IFNB or about 40× above background were observed. With cells treated with both ERNA-SARS2-101-m_IFNB and ERNA-SARS2-101-GDura, levels of GDura reached ~100× above background and levels of m_IFNB reached >30× above background, approximately the same levels that were observed in cells treated with only one encrypted RNA.

### Example 41: Treatment with Multiple Encrypted RNAs Having Different Translation Activators

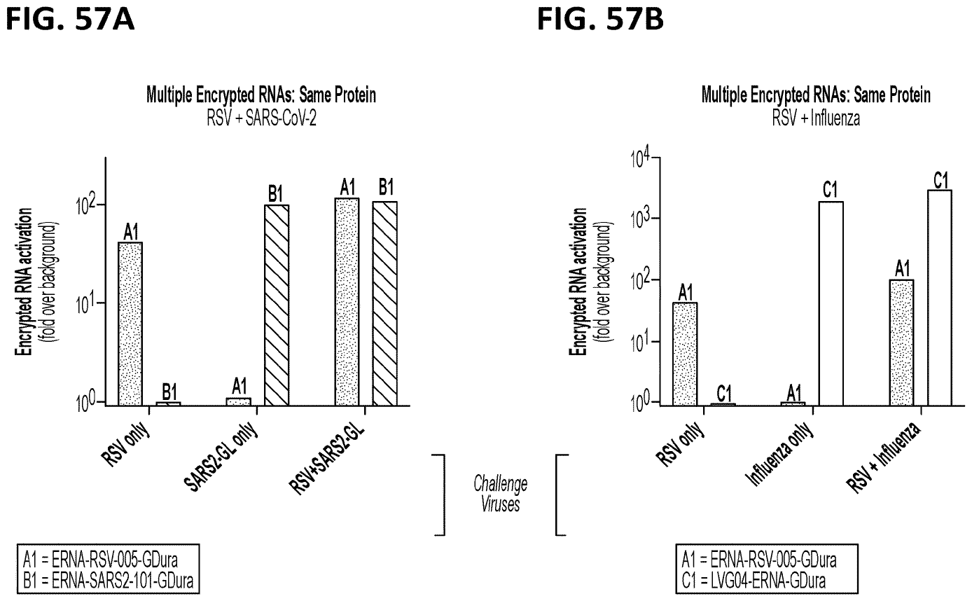

In FIG. 57B, experiments were conducted to test the specificity of encrypted RNA activation by target-specific translation activators when two or more distinct encrypted RNAs with distinct target-specific translation activators were applied to the same cell population and subsequently treated with a target-specific translation activator.

Human A549 cells or 293T cells were treated with influenza encrypted RNAs encoding GDura as described in Example 13, using LVG04-ERNA-GDura. At 0 hpi, the cultured cells were left uninfected or infected with influenza virus at an MOI of 0.1 (as described previously) or infected with RSV at an MOI of 0.1 (as described previously). RSV encrypted RNA encoding mouse interferon-beta (ERNA-RSV-005-m_IFNB) were applied to the cells at ~2 hpi.

At ~24, ~48, and ~72 hpi, the GDura or mouse IFNB containing supernatant was harvested. The amount of secreted luciferase measured by plate reader assay as described above. Secreted levels of mouse IFNB were quantified by ELISA as described above.

Populations of cells treated with both IAV encrypted RNA and RSV encrypted RNA and left uninfected showed no detectable levels of GDura or m_IFNB. Similarly, cells treated infected with IAV or RSV but not treated with encrypted RNAs showed no detectable levels of GDura or m_IFNB. In contrast, cells treated with both encrypted RNAs and infected with IAV secreted levels of GDura ~1000× above background levels and had undetectable levels of m_IFNB, owing to the specificity of the target-specific translation activator. Similarly, cells treated with both encrypted RNAs and infected with RSV secreted levels of m_IFNB ~30-100× above background, but had undetectable levels of GDura.

FIG. 57A shows a similar experiment performed in cells treated with ERNA-SARS2-101-GDura and ERNA-RSV-005-m_IFNB and infected with either RSV or generation-limited SARS-COV-2 (SARS2-GL from Example 19).

Populations of cells treated with both sarbecovirus encrypted RNA and RSV encrypted RNA and left uninfected showed no detectable levels of GDura or m_IFNB. Similarly, cells treated infected with generation-limited SARS-COV-2-GL (SARS2-GL) or RSV but not treated with encrypted RNAs showed no detectable levels of GDura or m_IFNB. In contrast, cells treated with both encrypted RNAs and infected with SARS2-GL secreted levels of GDura ~1000× above background levels and had undetectable levels of m_IFNB, owing to the specificity of the target-specific translation activator. Similarly, cells treated with both encrypted RNAs and infected with RSV secreted levels of m_IFNB ~100× above background, but had undetectable levels of GDura. No “cross-talk” was observed between different encrypted RNAs and their target-specific translation activators. Therefore, even in cells treated with multiple, distinct encrypted RNAs, an individual encrypted RNA can be selectively activated by the introduction of a target-specific translation activator of the encrypted RNA.

### Example 42: Activation of Henipavirus Encrypted RNAs by Providing Isolated mRNAs that can be Translated to Comprise a Target-Specific Translation Activator

We demonstrate here that Henipavirus encrypted RNAs can also be activated by providing a target-specific translation activator as a set of one or more IVT-produced mRNAs, which together reconstitute a target-specific translation activator.

Pools of three strain-specific purified messenger RNAs encoding Nipah virus (NiV) (NCBI GenBank ID: AY029767) or Hendra virus (HeV) (NCBI GenBank ID: AF017149) N, P and L proteins were produced using IVT as described above. HeV and NiV encrypted RNAs were prepared as described above, with scaffolds listed in Table 2. A representative NiV encrypted RNA is ERNA-NiV-001-GDura. A representative HeV encrypted RNA is ERNA-HeV-001-GDura. Both ERNA-NiV-001-GDura and ERNA-HeV-001-GDura are henipavirus encrypted RNAs.

Strain-specific mRNA pools were prepared, representing mixtures of strain-specific N mRNA, P mRNA and L mRNA at several different mass ratios. Two effective mass ratios were comprised of: (i) “mRNA pool_A” of 3.125:2:1 (N:P:L) or (ii) “mRNA pool_B” of 1:1:1 (N:P:L).

Human AD293 or 293T cells grown in 24-well plates (300K cells/well) were treated simultaneously by one of the following: (Treatment 1) no RNA; (Treatment 2) ~295 ng of ERNA-NiV-001-GDura or ~295 ng of ERNA-HeV-001-GDura; (Treatment 3) ~125 ng of ERNA-NiV-001-GDura or ~125 ng of ERNA-HeV-001-GDura; (Treatment 4) ~131.5 ng of “mRNA Pool_A”; (Treatment 5) ~375 ng of “mRNA Pool_B”; (Treatment 6) ~295 ng of ERNA-NiV-001-GDura or ~295 ng of ERNA-HeV-001-GDura, and ~131.5 ng of strain-specific “mRNA Pool_A”, or (Treatment 7)) ~125 ng of ERNA-NiV-001-GDura or ~125 ng of ERNA-HeV-001-GDura, and ~375 ng of strain-specific “mRNA Pool_B”.

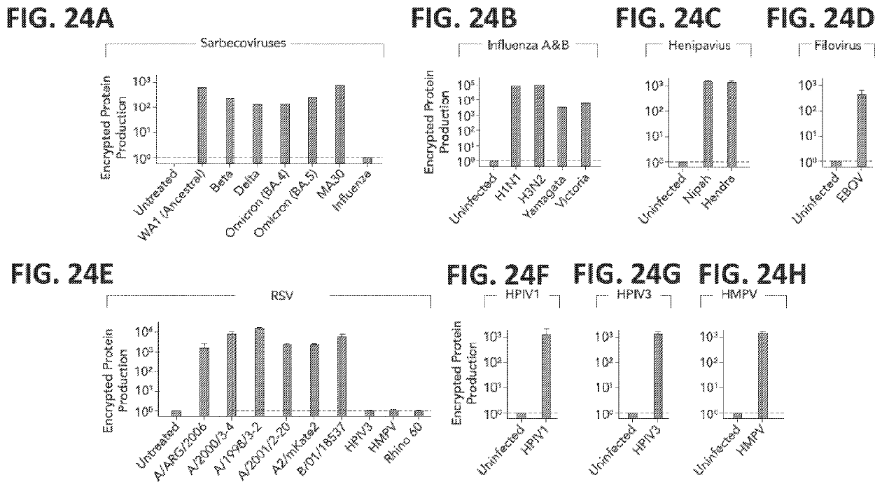

24C shows that a henipavirus encrypted RNA (ERNA-NiV-001-GDura) is activated by both Nipah virus translation activators and Hendra virus translation activators. Within FIG. 24C, “uninfected” corresponds to Treatment 3, “Nipah” corresponds to Treatment 6 when the mRNA pool is derived from Nipah virus proteins and “Hendra” corresponds to Treatment 6 when the mRNA pool is derived from Hendra virus proteins.

Additionally, ERNA-NiV-GD and ERNA-HeV-GD cross-activation experiment was performed with a combination of respective ERNA and strain-mismatched mRNA pool complex. The amount of the translated polypeptide of interest (secreted GDura luciferase) was quantified at 24 and 48 hours post-treatment as described previously. Translation activators from both viruses were able to activate their species-matched encrypted RNA and the non-species matched encrypted RNA.

### Example 43: Construction of Filovirus Encrypted RNAs

Filovirus encrypted RNAs were developed analogously to other encrypted RNAs (e.g. RSV encrypted RNAs) as described above. A listing of filovirus encrypted RNA scaffolds and corresponding L & R region sequences are provided in Table 2. Filovirus encrypted RNAs are have the prefix “ERNA-ZEBOV-” (Zaire ebolavirus), “ERNA-SEBOV-” (Sudan ebolavirus), or “ERNA-MARV-” (Marburg virus).

### Example 44: Activation of Ebolavirus Encrypted RNAs by Providing Isolated mRNAs that can be Translated to Comprise a Target-Specific Translation Activator

We demonstrate here that encrypted RNAs can also be activated by providing a target-specific translation activator as a set of one or more IVT-produced mRNAs, which together comprises a target-specific translation activator.

Pools of four strain-specific purified messenger RNAs encoding for Zaire Ebolavirus virus (ZEBOV) (NCBI GenBank ID: AF086833) were prepared using IVT. ZEBOV encrypted RNAs were prepared as described. One example is ERNA-ZEBOV-001-GDura.

Strain-specific “mRNA pools” (comprised of mRNA components selected from NP, VP30, VP35 and L) were prepared: “mRNA pools” were comprised of a mixture of strain-specific NP mRNA, VP30 mRNA, VP35 mRNA and L mRNA at standard mass ratio of 1:1:1:1.

Human AD293 or 293T cells in 24-well plates (300K/well) were treated simultaneously by one of the following: (Treatment 1) no RNA; (Treatment 2) ~100 ng of ERNA-ZEBOV-001-GDura; (Treatment 3) ~400 ng of “mRNA Pool”, or (Treatment 4) ~100 ng of ERNA-ZEBOV-001-GDura and ~400 ng of strain-specific “mRNA Pool”. The amount of the translated polypeptide of interest (secreted GDura luciferase) was quantified at 24 and 48 hours post-treatment as described previously.

FIG. 24D shows that ERNA-ZEBOV-001-GDura is activated by “mRNA Pool”, comprising an ebolavirus polymerase complex. Treatment 2 corresponds to “Uninfected” and Treatment 4 corresponds to “EBOV”. GDura levels were background in Treatment 1 or Treatment 3 (not shown).

### Example 45: (Prophetic) Activate Marburg Virus Encrypted RNAs or Sudan Ebolavirus Encrypted RNAs by Providing Isolated mRNAs that can be Translated to Comprise a Target-Specific Translation Activator

Prepare pools of four strain-specific purified messenger RNAs encoding for Marburgvirus (MARV) (NCBI GenBank ID: NC_001608.3) proteins NP, VP30, VP35 and L by IVT. Similarly, prepare Sudan ebolavirus purified mRNA corresponding to Sudan ebolavirus (NP, VP30, VP35, and L mRNA) (sequences from NCBI GenBank ID: FJ968794. Prepare suitable filovirus encrypted RNA suitable for activation by Marburg virus (e.g. ERNA-MARV-001, ERNA-MARV-002, ERNA-MARV-003 in Table 2) or Sudan ebolavirus (e.g. ERNA-SEBOV-001, ERNA-SEBOV-002, ERNA-SEBOV-003, ERNA-SEBOV-004), encoding a reporter polypeptide of interest (such as GDura).

Prepare a strain-specific “mRNA pool” (comprising mRNA components selected from NP, VP30, VP35 and L from MARV or NP, VP30, VP35 and L from Sudan ebolavirus). Form an “mRNA pool” comprising a mixture of strain-specific NP mRNA, VP30 mRNA, VP35 mRNA and L mRNA at standard mass ratio of 1:1:1:1.

To quantify activation of Marburg virus encrypted RNAs, treat human AD293 or 293T cells in 24-well plates (300K/well) with one of the following: (Treatment 1) no RNA; (Treatment 2) ~100 ng of a suitable Marburg virus encrypted RNA encoding GDura; (Treatment 3) ~400 ng of strain-specific “mRNA Pool”, or (Treatment 4) ~100 ng of a suitable Marburg virus encrypted RNA encoding GDura and ~400 ng of strain-specific “mRNA Pool”.

To quantify activation of Sudan ebolavirus encrypted RNAs, treat human AD293 or 293T cells in 24-well plates (300K/well) with one of the following: (Treatment 1) no RNA; (Treatment 2) ~100 ng of a suitable Sudan ebolavirus encrypted RNA encoding GDura; (Treatment 3) ~400 ng of strain-specific “mRNA Pool”, or (Treatment 4) ~100 ng of a suitable Sudan ebolavirus encrypted RNA encoding GDura and ~400 ng of strain-specific “mRNA Pool”.

Quantify the level of polypeptide of interest (e.g. secreted GDura luciferase) at 24 and 48 hours post-treatment as described above and interpret activation as described above (e.g., as in Example 44).

### Example 46: (Prophetic) Activation of Newcastle Disease Virus Encrypted RNAs can be Performed by Providing Isolated mRNAs that can be Translated to Comprise a Target-Specific Translation Activator

Newcastle disease virus (NDV) also known as avian paramyxovirus type 1 is a member of the family Paramyxoviridae with a non-segmented negative-stranded RNA genome.

For NDV encrypted RNA some L and R flanking nucleotide sequences can be obtained by concatenation of nucleotide regions from publicly available NDV strain La Sota (NCBI GenBank ID: AF077761.1) sequence. A 191 nt region from the 5′ end of the NDV genomic RNA (viral RNA) (5′ trailer of L gene end sequence and NS1 gene start) can be used as the L flank sequence for NDV encrypted RNA (antis_5p_NDV, SEQ ID NO: 245). The terminal 121 nt from the 3′ end of the NDV genomic RNA consisting of the untranslated region, gene start, and 3′-Leader sequences can be used as the R flank for NDV encrypted RNA (antis_3p_NDV, SEQ ID NO: 248).

The initial NDV antisense encrypted RNA scaffolds can be designed by combining three independent sequence blocks described above. For example, ERNA-NDV-001-GDura can be generated by concatenation of antis_5p_NDV (SEQ ID NO: 245), the reverse complement of the coding sequence of GDura (rcCDS_GDura, SEQ NO: 301), and antis_3p_NDV (SEQ ID NO: 248) in 5′ to 3′ order. Additional suitable encrypted RNA scaffolds can be prepared for testing (e.g. ERNA-NDV-002-GDura, ERNA-NDV-003-GDura).

Pools of three strain-specific purified messenger RNAs encoding for NDV (NCBI GenBank ID: AF077761.1) proteins N, P and L can be prepared using IVT. NDV encrypted RNAs can be prepared as described.

Strain-specific “mRNA pools” representing mixtures of strain-specific N mRNA, P mRNA and L mRNA at different mass ratios should be prepared, e.g.: (i) “mRNA pool_A” of 2:1:1 (N:P:L) and (ii) “mRNA pool_B” of 1:1:1 (N:P:L). The mass of NDV encrypted RNA (encoding a reporter polypeptide) can be 100 ng for “mRNA pool_A” and 125 ng for “mRNA pool_B”.

Human AD293 or 293T or CER cells grown in 24-well plates (300K cells/well) can be simultaneously transfected with Lipofectamine MessengerMax with one of the following: (Treatment 1) no RNA; (Treatment 2) ~100 ng of NDV encrypted RNA (encoding a reporter polypeptide); (Treatment 3) ~125 ng of NDV encrypted RNA (encoding a reporter polypeptide); (Treatment 4) ~400 ng of “mRNA Pool_A”; (Treatment 5) ~375 ng of “mRNA Pool_B”; (Treatment 6) ~100 ng of NDV encrypted RNA (encoding a reporter polypeptide) and ~400 ng of strain-specific “mRNA Pool_A”, or (Treatment 7) ~125 ng of NDV encrypted RNA (encoding a reporter polypeptide) and ~375 ng of strain-specific “mRNA Pool_B”. The amount of the translated polypeptide of interest (secreted GDura luciferase) can be quantified and interpreted at 24 and 48 hours post-treatment as described previously.

### Example 47: Identification of Alternative and Additional L Region and R Region Sequences by Bioinformatic Analysis of Viral Genome Sequences

Identified L region sequences and R region sequences of encrypted RNAs were used as query sequences to search genomic databases for viral sequences related to reference L and R sequences. The United States National Institutes of Health GenBank database was searched using BLAST (Basic Local Alignment Search Tool. The non-redundant “nr/nt” nucleotide database was queried using the “megablast” and “blastn” algorithms using some L or R sequences as query sequences. High-scoring pairs were filtered to those which met the the criteria: (i) expected value <0.001; (ii) sequence name matched a closely related virus to the viral target of the encrypted RNA. Up to about 5,000 alignments were analyzed for each query sequence. Pairwise point mutations were identified and tabulated if at least two variant sequences were detected with the same unambiguous variation with respect to the reference sequences.

Variants in Table 17 are identified as “(reference position) (variant)” notation. As an example, a variant identified as “25a” would indicate that the nucleotide at position 25 of the reference sequence is not an “a”, but can be converted to the variant sequence by changing the nucleotide at position 25 to an “a” (“c”, “g”, and “t” are noted analogously). Where “A” is indicated, the variation is a deletion of the nucleotide at the reference position, e.g., “304” indicates that the nucleotide at reference position 30 is deleted in the variant. Insertions are marked by the location of the positions of the flanking nucleotide of the insertion: “21_22ins_t” indicates that the variant has an insertion of “t” between the nucleotides located at position 21 and position 22 of the reference sequence.

The variations identified in Table 17 are putatively associated with functional viral genomes, which must successfully undergo repeated rounds of replication to be amplified and isolated for sequencing. Taken together, both the source of the genomic information (“functional viruses”) and their relationship to some known L and R flanking sequences, implies that the variants identified in Table 17 are functional.

The relationship between the variations recorded in Table 17 and additional L and R sequences not listed in Table 17 can easily be inferred to a person of ordinary skill in the art by aligning a related L or R sequence to those listed in Table 17, and then subsequently mapping the variation onto the distinct sequence not in Table 17.

TABLE 17
Variants of L and R sequences identified by bioinformatic analysis of viral genomic sequences.

| SEQ | | Number | |
|---|---|---|---|
| ID | Name of L Region or R | Variants | |
| NO | Region Name | Identified | Variants |
| 1 | antis_5p_IAV | 71 | 5c, 12Δ, 13Δ, 14Δ, 15c, 15Δ, 15g, 16a, 16Δ, 16t, 17g, 18a, 18c, 18g, 19g, 21Δ, 22Δ, 22t, 23a, 23Δ, 23g, 23t, 24a, 24c, 24Δ, 24g, 25a, 25Δ, |
| | | | 25t, 26g, 26t, 27a, 27c, 27g, 28c, 28g, 28t, 29a, 29c, 29Δ, 29g, 30Δ, 30g, 30t, 31a, 31c, 32a, 32t, 33a, 33c, 33Δ, 34a, 35g, 35t, 36g, 36t, 37c, |
| | | | 37g, 37t, 38a, 38c, 39c, 39g, 40a, 40t, 41a, 41c, 42g, 42t, 43g, 43t |
| 2 | antis_5p_IAV_seg01 | 4 | 30t, 30a, 31a, 31t |
| 4 | antis_5p_IAV_seg03 | 37 | 4t, 9t, 9g, 31c, 31a, 35c, 37a, 38t, 38a, 39g, 39c, 40g, 41g, 41c, 42c, 42g, 43t, 43g, 45c, 46g, 47t, 48t, 49g, 49t, 50t, 50a, 50g, 50Δ, 53c, 53t, |
| | | | 54t, 55t, 55c, 56t, 56a, 5_6ins_a, 22_23ins_t |
| 10 | antis_5p_IBV | 186 | 7g, 7t, 8g, 9g, 11c, 13t, 13c, 13Δ, 14t, 15t, 15c, 16g, 16c, 16t, 17c, 17t, 17g, 17Δ, 18t, 18Δ, 18g, 18c, 19c, 19g, 19t, 20c, 20t, 20g, 21c, 21g, |
| | | | 21t, 22c, 22t, 22a, 23c, 23g, 23t, 23a, 24Δ, 24g, 24t, 24c, 25t, 25a, 25c, 25g, 26t, 26g, 26c, 27a, 27t, 27c, 27g, 28c, 28g, 28t, 29c, 29t, 29g, |
| | | | 30g, 30c, 30t, 30a, 30Δ, 31g, 31c, 31a, 31t, 31Δ, 32c, 32t, 32Δ, 32g, 33Δ, 33t, 33c, 33g, 34c, 34t, 35a, 35c, 35t, 35g, 36c, 36a, 36g, 37c, 37t, |
| | | | 37g, 38g, 38c, 38t, 39c, 39t, 39g, 40Δ, 40a, 40t, 40g, 41c, 41g, 42g, 42c, 42t, 43g, 43t, 43c, 43a, 44c, 44t, 44g, 45c, 45g, 45t, 46t, 46a, 47c, |
| | | | 47g, 48c, 48g, 48t, 49t, 49a, 49c, 50c, 51c, 51g, 52c, 53g, 53c, 54c, 54g, 55g, 55c, 55t, 56a, 56g, 56Δ, 57a, 57c, 57g, 57t, 58g, 58c, 59t, 59g, |
| | | | 59a, 59c, 60t, 60a, 60g, 60c, 61a, 61t, 61c, 61g, 62c, 62a, 62t, 63g, 63c, 63a, 64t, 64c, 65c, 65t, 66a, 66c, 66g, 67t, 68t, 68g, 68a, 69t, 69a, |
| | | | 70c, 70t, 71c, 72g, 72c, 73g, 77c, 81a, 84t, 85c, 89t |
| 11 | antis_5p_IBV_seg01 | 187 | 5c, 6t, 6c, 7c, 8c, 8a, 8g, 8t, 9g, 9t, 10g, 10c, 10t, 11c, 11a, 11g, 12c, 12g, 12t, 13c, 14g, 14c, 14t, 15g, 15t, 15a, 15c, 16g, 16t, 16a, 16c, 17t, |
| | | | 17c, 18g, 18c, 18a, 18t, 19c, 19t, 20c, 20g, 20t, 20a, 21c, 21Δ, 21g, 21t, 22a, 22t, 22c, 22g, 23g, 23c, 23a, 23t, 24a, 24t, 24c, 24g, 25c, 25a, |
| | | | 25t, 25g, 26g, 26c, 26a, 27c, 27t, 27a, 28t, 28c, 28a, 29a, 29t, 29g, 29c, 30t, 30c, 30g, 30a, 31g, 31c, 31t, 32t, 32c, 32a, 33t, 33a, 33g, 33c, |
| | | | 34c, 34t, 34g, 35g, 35t, 35c, 36c, 36g, 37c, 37g, 37t, 38t, 38c, 38a, 38g, 39a, 39t, 39g, 40c, 40a, 40g, 41c, 41a, 41g, 41t, 42c, 42a, 42g, 43g, |
| | | | 43a, 43c, 44a, 44c, 44g, 44t, 45a, 45c, 45g, 45t, 46c, 46g, 46a, 47g, 47c, 48t, 48c, 48g, 49t, 49g, 50c, 50g, 51c, 51g, 51t, 52t, 52c, 52g, 53g, |
| | | | 53t, 54t, 54g, 55t, 55g, 56t, 56g, 56c, 57t, 57g, 58g, 58t, 59c, 59g, 60g, 60t, 61g, 61t, 62g, 62c, 62t, 63t, 63c, 63g, 64g, 64t, 65a, 65g, 66t, |
| | | | 66c, 68c, 69t, 70g, 71g, 72g, 74g, 75g, 76g, 22_23ins_t |
| 12 | antis_5p_IBV_seg02 | 83 | 6t, 7t, 9t, 9a, 10c, 10t, 10a, 11a, 11t, 12t, 12c, 12a, 13g, 13a, 13t, 13c, 14c, 14t, 14a, 15c, 15a, 15g, 15t, 16c, 16a, 16t, 17t, 17g, 17a, 17c, 18t, |
| | | | 18a, 19c, 19g, 20c, 20t, 21a, 21t, 22t, 22g, 23a, 23c, 24t, 25a, 25t, 26t, 26g, 27t, 27a, 27g, 28t, 28a, 28c, 29a, 29t, 30a, 30g, 31g, 31c, 32t, |
| | | | 32a, 32c, 32g, 33a, 33t, 33c, 34a, 34c, 35a, 35c, 35g, 36t, 36c, 37c, 37g, 38c, 38g, 39c, 40a, 40c, 41c, 55c, 21_22ins_t |
| 13 | antis_5p_IBV_seg03 | 134 | 6t, 7g, 7t, 8g, 9a, 9t, 10c, 10t, 10a, 11t, 11a, 12c, 13a, 13Δ, 14t, 14a, 15t, 15c, 15Δ, 16t, 16Δ, 17g, 17Δ, 18g, 18Δ, 18a, 19g, 19t, 19c, 19Δ, |
| | | | 21g, 21a, 22a, 22Δ, 22g, 23c, 23a, 23Δ, 24g, 24Δ, 25a, 25c, 25Δ, 28c, 28a, 29t, 29a, 30t, 30a, 33a, 36t, 36a, 37a, 38Δ, 43g, 44g, 44c, 50t, |
| | | | 50g, 50a, 51a, 51g, 51t, 52g, 52c, 52t, 53c, 53g, 53a, 53t, 54c, 54t, 55g, 55t, 56a, 56t, 56g, 57c, 57g, 57t, 58g, 58t, 59t, 59a, 59c, 59g, 60a, |
| | | | 60t, 61a, 61t, 61g, 62t, 62g, 63g, 63t, 64c, 64g, 64t, 65g, 65t, 66a, 66t, 66g, 67g, 67t, 68g, 68t, 68c, 69g, 69c, 70g, 70c, 70t, 71g, 71t, 72t, |
| | | | 73t, 73g, 74g, 74t, 74c, 74a, 75t, 75g, 76c, 76t, 82t, 85g, 86t, 87t, 87g, 90g, 17_18ins_t, 23_24ins_t |
| 14 | antis_5p_IBV_seg05 | 162 | 5c, 9g, 9c, 9a, 10g, 10c, 10a, 11g, 11c, 12g, 12c, 12Δ, 13t, 13Δ, 13c, 13a, 13g, 14c, 14g, 14Δ, 14t, 15g, 15c, 15Δ, 16g, 17c, 17a, 17g, 18g, |
| | | | 18a, 18c, 18Δ, 19g, 19c, 20g, 20t, 20c, 20Δ, 21g, 21t, 21c, 21Δ, 22g, 23g, 23c, 24c, 24g, 25g, 27a, 29a, 29g, 31g, 31a, 33c, 33a, 33g, 33t, |
| | | | 34g, 34t, 35c, 35t, 36c, 36g, 37g, 37c, 37t, 38g, 39g, 39c, 39a, 39t, 41g, 41a, 41c, 41t, 42t, 42a, 43a, 43g, 44c, 45c, 45g, 45t, 46g, 46c, 46t, |
| | | | 46a, 47c, 47a, 47g, 48a, 48c, 48g, 49g, 50c, 50g, 50t, 51c, 51g, 52t, 52a, 52g, 53t, 53g, 54t, 54g, 55t, 55c, 56t, 56c, 58g, 59t, 59c, 59g, 60a, |
| | | | 60g, 60c, 60t, 61t, 61c, 62t, 63t, 63g, 63c, 64g, 64a, 64c, 64t, 65c, 65g, 65t, 66t, 66g, 68g, 68a, 69g, 69t, 70t, 71t, 71c, 72t, 72g, 73g, 73c, |
| | | | 74t, 74g, 74a, 74c, 75c, 76g, 76c, 77a, 77c, 78c, 78t, 80c, 80g, 80t, 81t, 81a, 11_12ins_t, 11_12ins_a |
| 15 | antis_5p_IBV_seg06 | 200 | 6a, 6c, 7g, 8g, 9a, 10c, 10t, 10g, 11g, 12c, 12g, 12t, 13g, 13t, 13c, 13a, 14c, 14g, 15c, 15g, 16a, 16c, 16t, 17c, 17g, 17a, 17t, 18c, 18g, 19t, |
| | | | 19c, 19g, 20c, 20a, 20t, 21c, 21g, 22g, 22c, 22t, 23c, 23Δ, 23g, 23a, 24c, 24g, 25t, 25a, 25g, 26a, 26c, 27g, 27c, 27a, 28t, 28a, 28c, 28g, 29c, |
| | | | 29g, 29t, 29a, 30c, 30g, 30t, 30a, 31c, 31g, 32c, 32a, 32t, 33a, 33c, 33t, 33g, 34a, 34t, 34g, 35a, 35c, 35g, 36g, 36c, 36a, 37g, 37c, 37a, 37t, |
| | | | 38c, 38a, 39c, 39t, 40a, 40g, 40c, 40t, 41g, 41t, 42t, 42c, 42g, 43g, 43t, 43c, 44a, 44t, 45a, 45t, 46t, 46g, 47t, 47a, 47g, 48c, 48t, 48a, 49g, |
| | | | 49t, 50g, 51a, 51t, 52t, 52c, 52a, 53a, 53t, 53g, 54t, 54g, 55g, 55t, 56t, 56a, 56g, 57t, 57g, 58t, 59t, 59a, 62t, 62g, 62Δ, 63t, 63c, 63a, 64t, |
| | | | 64g, 64c, 65g, 65t, 66a, 66t, 67g, 68t, 68g, 69t, 69a, 71t, 71a, 72t, 72g, 73a, 73t, 74t, 74c, 75t, 75a, 75g, 76g, 77c, 77g, 78t, 78c, 78a, 79c, |
| | | | 79t, 80a, 80c, 80t, 80g, 81a, 81g, 81c, 82t, 82c, 83g, 83t, 84t, 87t, 88t, 88c, 89t, 89c, 90t, 90c, 94g, 95c, 96c, 97c, 98t |
| 16 | antis_5p_IBV_seg07 | 172 | 6c, 7a, 7c, 8t, 8c, 9a, 9c, 9t, 10c, 10a, 10t, 11g, 11c, 11a, 11t, 11Δ, 12c, 12t, 12a, 13c, 13a, 13g, 14a, 14c, 15a, 15t, 16a, 16t, 16c, 16g, 17c, |
| | | | 17a, 17g, 18a, 18t, 18c, 19c, 19g, 20g, 20a, 21a, 21g, 21t, 21c, 21Δ, 22c, 23c, 23g, 24a, 24t, 24c, 24g, 25g, 25c, 26c, 26g, 27c, 27g, 28a, |
| | | | 28g, 28t, 29g, 30a, 30c, 30g, 31g, 31c, 32c, 33a, 33c, 33g, 34a, 34c, 36t, 36c, 37c, 37a, 38c, 38g, 40c, 40a, 41a, 41c, 41g, 42a, 42g, 42c, |
| | | | 43g, 43a, 43c, 44t, 44c, 44a, 44g, 45c, 45g, 45a, 46Δ, 46t, 46c, 46g, 46a, 47g, 47a, 47c, 48a, 48c, 48g, 49c, 49g, 50g, 50a, 50t, 51t, 51g, 51c, |
| | | | 52t, 52a, 52g, 52c, 53g, 53c, 53t, 54g, 54a, 54c, 55g, 55c, 55Δ, 56a, 56c, 56g, 57g, 57c, 58g, 58t, 58a, 58c, 59g, 59a, 59c, 60a, 60t, 60c, 61g, |
| | | | 61c, 61a, 62g, 62a, 62t, 63g, 64a, 64g, 64c, 65g, 65c, 66a, 66c, 66g, 67c, 67a, 68c, 68a, 69g, 73g, 28_29ins_t, 28_29ins_g, 49_50ins_g, |
| | | | 54_55ins_g, 56_57ins_g, 59_60ins_g, 60_61ins_g |
| 18 | antis_3p_IAV | 46 | 3c, 3g, 4a, 4c, 4t, 5a, 5c, 5t, 6a, 6c, 6g, 8a, 8Δ, 8t, 8c, 9c, 9g, 9a, 10a, 11g, 11a, 14g, 17a, 18a, 12a, 12Δ, 13a, 13c, 13Δ, 13g, 14c, 14t, 15a, |
| | | | 15Δ, 16c, 17c, 18c, 18Δ, 18g, 19a, 19g, 19t, 22t, 25t, 27g, 29c |
| 44 | antis_3p_IBV | 9 | 27a, 10g, 19c, 22a, 22c, 23a, 24c, 29t, 30g |
| 52 | antis_3p_IBV_seg05 | 65 | 5g, 6c, 7c, 7g, 8c, 10t, 11g, 12a, 12c, 13c, 14c, 14Δ, 15t, 15a, 15Δ, 16c, 16Δ, 16t, 17c, 17Δ, 17a, 18c, 18g, 18Δ, 19c, 19g, 19a, 19Δ, 20g, 20t, |
| | | | 20Δ, 21t, 21a, 21g, 21Δ, 22c, 22Δ, 23a, 23Δ, 24a, 25c, 25a, 26a, 26t, 27t, 27a, 29c, 29g, 30t, 30a, 31g, 32g, 32c, 33a, 34c, 35c, 36t, 37g, 37c, |
| | | | 38t, 38a, 42g, 52a, 9_10ins_n, 19_20ins_t |
| 54 | antis_3p_IBV_seg06 | 17 | 10g, 12t, 12c, 14t, 14a, 16t, 21c, 21g, 22c, 25t, 25a, 30t, 30a, 35g, 35c, 20_21ins_t, 24_25ins_t |
| 58 | antis_3p_IBV_seg08 | 7 | 5t, 6t, 10g, 23g, 31g, 33c, 40t |
| 60 | sense_5p_SARS1 | 1522 | 3t, 4a, 5a, 6a, 6t, 7a, 7c, 8t, 8c, 10a, 10t, 11t, 12t, 12a, 13t, 16t, 18t, 20c, 21a, 21c, 22a, 23a, 24a, 27t, 27a, 30c, 31t, 31a, 32t, 33t, 34t, 36t, |
| | | | 37t, 45a, 48t, 55g, 63a, 66a, 67t, 69g, 79g, 80g, 81g, 81a, 82g, 82a, 85g, 86a, 87a, 88a, 88t, 89a, 90a, 92a, 100t, 101t, 102t, 102a, 103t, |
| | | | 106c, 106t, 109t, 110t, 110c, 111t, 111c, 112t, 112c, 113c, 115t, 116c, 122t, 123t, 124t, 125t, 125c, 126t, 126c, 128t, 129Δ, 129t, 129c, |
| | | | 130Δ, 130c, 130t, 131c, 131t, 132c, 132t, 133t, 133Δ, 134Δ, 135Δ, 135c, 136Δ, 136t, 137Δ, 139Δ, 140Δ, 141a, 148g, 150g, 151g, 152g, |
| | | | 153g, 153c, 153a, 154c, 155c, 156c, 156g, 156t, 159c, 168t, 169t, 169a, 170t, 170a, 171t, 171a, 171g, 172a, 174t, 174g, 175a, 176g, 176a, |
| | | | 177c, 180g, 180t, 181g, 182g, 182t, 183g, 186g, 186a, 195g, 203t, 204c, 204t, 208c, 209c, 210c, 211c, 214c, 216t, 219t, 221c, 222c, 223c, |
| | | | 223t, 224c, 224t, 225t, 226t, 227c, 227a, 229t, 229a, 232t, 233t, 234t, 235t, 235Δ, 237t, 240t, 246t, 249t, 250t, 252t, 264a, 267c, 271c, 272c, |
| | | | 273c, 274c, 274t, 275c, 276c, 276a, 277c, 278t, 279t, 279c, 280t, 281t, 281a, 282a, 284t, 284c, 285g, 286a, 287t, 292g, 294a, 295c, 300c, |
| | | | 309t, 316t, 317t, 318t, 319t, 319a, 320t, 320a, 321a, 322a, 322c, 322t, 323t, 324c, 324t, 325a, 326c, 327c, 327t, 328c, 328t, 329c, 329t, |
| | | | 330c, 330t, 331c, 331t, 332c, 333c, 333a, 334c, 337c, 338c, 339c, 339t, 340c, 340a, 340t, 341a, 342a, 343a, 343c, 346a, 349t, 350t, 351t, |
| | | | 351a, 352t, 352a, 353a, 354c, 354a, 355a, 355t, 358a, 358c, 359c, 360c, 360g, 361c, 364c, 364g, 365t, 365g, 366g, 367g, 367t, 367c, 369t, |
| | | | 370t, 370g, 371t, 372t, 373t, 374t, 375t, 376a, 377a, 377t, 378a, 378g, 378t, 379a, 382c, 382a, 383g, 384t, 386c, 387t, 387c, 388c, 389c, |
| | | | 390t, 391t, 392t, 392c, 393t, 394t, 395t, 396t, 397t, 397c, 398g, 398a, 399g, 400g, 400t, 401g, 402a, 404g, 405t, 406g, 410t, 411c, 412c, |
| | | | 413c, 413t, 414c, 414t, 415c, 415t, 415a, 416t, 416Δ, 418g, 418c, 419t, 421a, 422a, 422g, 423a, 423g, 424a, 424g, 424t, 425g, 425t, 425a, |
| | | | 426t, 427t, 427a, 427c, 428g, 429t, 430t, 436t, 437t, 438t, 439t, 439c, 440t, 442t, 443t, 444t, 445t, 445a, 446a, 447a, 448a, 448t, 451a, 454g, |
| | | | 456c, 469c, 470c, 471c, 472c, 474g, 474t, 475c, 477t, 478g, 479g, 479a, 480g, 480t, 481g, 481c, 481a, 484g, 484t, 485t, 485c, 486t, 486c, |
| | | | 486g, 487t, 487c, 487g, 488c, 488g, 488a, 489g, 489a, 489t, 490Δ, 490t, 491Δ, 491c, 492g, 492t, 494Δ, 495t, 495Δ, 496Δ, 496g, 497Δ, |
| | | | 498Δ, 498t, 499t, 499g, 500t, 500Δ, 501t, 501Δ, 501c, 502t, 502Δ, 503Δ, 503t, 504t, 505t, 506t, 506g, 507t, 507Δ, 507g, 507a, 508t, 508g, |
| | | | 508Δ, 509g, 509t, 509Δ, 509a, 510t, 510Δ, 510a, 511t, 511a, 511g, 512a, 512t, 512g, 512Δ, 513g, 513t, 513Δ, 514g, 514Δ, 514t, 514a, |
| | | | 515a, 517g, 519a, 519t, 523a, 524a, 525a, 525g, 526a, 526c, 526g, 529a, 530c, 531a, 531c, 532c, 532t, 533c, 534c, 534t, 535c, 535a, 535t, |
| | | | 536a, 536c, 537a, 537t, 538a, 538c, 540t, 541a, 544c, 546a, 549a, 555a, 556g, 556t, 557a, 557t, 558t, 558g, 559t, 560g, 561g, 561a, 562g, |
| | | | 562a, 562t, 563g, 563a, 564a, 564g, 564t, 565g, 565Δ, 566g, 567t, 567a, 568g, 568t, 568Δ, 569t, 570t, 570c, 571t, 572t, 573t, 574t, 574c, |
| | | | 575c, 575t, 576c, 576t, 577c, 577t, 578t, 579t, 579Δ, 580t, 580c, 581c, 582c, 582t, 583c, 583t, 584t, 585t, 586t, 586c, 588a, 589t, 591a, |
| | | | 597t, 598g, 598t, 598a, 599t, 599a, 600t, 600a, 600g, 601a, 602g, 603t, 603g, 604g, 604a, 605g, 606g, 607g, 607t, 608t, 608g, 609t, 610t, |
| | | | 610g, 612g, 612a, 613t, 615g, 616g, 617g, 618g, 618a, 619g, 621a, 622g, 622t, 624a, 627a, 643a, 644a, 645a, 645t, 646a, 649a, 649t, 650t, |
| | | | 651t, 651c, 652t, 655t, 655c, 656c, 657c, 657g, 658c, 661c, 661g, 662g, 662t, 663t, 663g, 663a, 664g, 664t, 664c, 665c, 666c, 667c, 667t, |
| | | | 668g, 669g, 669c, 670g, 670c, 671g, 671c, 672c, 674g, 675c, 682a, 683a, 684a, 684t, 684g, 685a, 685t, 686t, 687t, 688a, 688Δ, 690c, 690t, |
| | | | 694c, 695c, 696c, 697c, 700c, 715t, 716t, 717t, 717a, 718t, 718a, 719t, 719a, 720a, 720Δ, 721t, 722t, 723a, 726t, 727g, 727t, 727c, 728t, |
| | | | 728c, 729t, 729c, 730c, 731c, 731g, 732g, 732t, 733g, 734g, 734c, 737g, 748a, 749a, 750a, 751a, 752a, 753a, 754a, 755a, 758t, 758a, 762t, |
| | | | 763t, 764t, 765t, 765Δ, 766Δ, 766t, 767Δ, 767a, 768Δ, 768t, 769Δ, 771Δ, 3_4ins_a, 16_17ins_t, 17_18ins_t, 17_18ins_n, 18_19ins_t, |
| | | | 18_19ins_c, 18_19ins_w, 18_19ins_r, 19_20ins_t, 19_20ins_c, 19_20ins_n, 20_21ins_c, 20_21ins_t, 21_22ins_c, 21_22ins_t, 21_22ins_a, |
| | | | 22_23ins_t, 22_23ins_c, 22_23ins_a, 24_25ins_c, 33_34ins_t, 34_35ins_t, 36_37ins_t, 406_407ins_t, 484_485ins_c, 484_485ins_t, |
| | | | 486_487ins_t, 488_489ins_c, 488_489ins_t, 489_490ins_c, 489_490ins_t, 490_491ins_c, 490_491ins_t, 491_492ins_c, 491_492ins_t, |
| | | | 494_495ins_c, 494_495ins_t, 560_561ins_g, 561_562ins_g, 585_586ins_a, 762_763ins_t, 763_764ins_t, 764_765ins_t, 765_766ins_t, |
| | | | 768_769ins_t, 9a, 9c, 9g, 10c, 10g, 12g, 15a, 16a, 16g, 17a, 18c, 18g, 19a, 21t, 24t, 25g, 26c, 27g, 29a, 30t, 35g, 35t, 36g, 37c, 38g, 38t, |
| | | | 39a, 39t, 40c, 41t, 42t, 45t, 51c, 52g, 53t, 56a, 58t, 61g, 63t, 66t, 67c, 68g, 70c, 70g, 71g, 72t, 75c, 75g, 77g, 84c, 87g, 89t, 94a, 97t, 98a, |
| | | | 99a, 108t, 117t, 118c, 121a, 127c, 137c, 138t, 140c, 141c, 142a, 143c, 145c, 146t, 147g, 151a, 152c, 157g, 157t, 158g, 159a, 161c, 161t, |
| | | | 163g, 165g, 167c, 172g, 173t, 176t, 177a, 177g, 177t, 178c, 179t, 184c, 185t, 188g, 189t, 190g, 192t, 194c, 195c, 201c, 202t, 209t, 213t, |
| | | | 216c, 217t, 218a, 220c, 221a, 221t, 223a, 229c, 231t, 232a, 234g, 239c, 240a, 241a, 256g, 257g, 259a, 278c, 281c, 285a, 286c, 286t, 290g, |
| | | | 291t, 314a, 318a, 325t, 327a, 327g, 334g, 334t, 336c, 336t, 343t, 345c, 345t, 348a, 354t, 357a, 357t, 360a, 361a, 363t, 364a, 366c, 369a, |
| | | | 372g, 375a, 377g, 379t, 381c, 382g, 383a, 384a, 387a, 388a, 394c, 399t, 402g, 402t, 403c, 404t, 405g, 407g, 408c, 417c, 420c, 421t, 426c, |
| | | | 426g, 429a, 430g, 432t, 435g, 438g, 441a, 441t, 442a, 443c, 444c, 444g, 447c, 449g, 449t, 450a, 450g, 450t, 453a, 455c, 460g, 462a, 465a, |
| | | | 465t, 468c, 470g, 473g, 475g, 477c, 483a, 483c, 489c, 493c, 493g, 494a, 494c, 494g, 495g, 496c, 497a, 500g, 502g, 503c, 503g, 504c, |
| | | | 504g, 515g, 515t, 516a, 516t, 517a, 517t, 519g, 520a, 520c, 520t, 521c, 521g, 522a, 522c, 522g, 526t, 528a, 528t, 531g, 533t, 534g, 537g, |
| | | | 538t, 539a, 540a, 540c, 543a, 543t, 546t, 547g, 548c, 549c, 549g, 551g, 554g, 554t, 555t, 558c, 569a, 569c, 570g, 578a, 582g, 585a, 588c, |
| | | | 588t, 591c, 591t, 592t, 594c, 597a, 605t, 606a, 606t, 609g, 612c, 615t, 618t, 621t, 623c, 623g, 624g, 625a, 630c, 633c, 634t, 636c, 641g, |
| | | | 642t, 648c, 651a, 652a, 653c, 654t, 657t, 660c, 663c, 667g, 669a, 669t, 671t, 676g, 677c, 680c, 682t, 686g, 690a, 690g, 692t, 693c, 696t, |
| | | | 702c, 704g, 704t, 706a, 707g, 707t, 708a, 709t, 710a, 711a, 711g, 712a, 712t, 713a, 713c, 714t, 717c, 723t, 724g, 724t, 725a, 726c, 729g, |
| | | | 732c, 734t, 735c, 736c, 739g, 740t, 741c, 742c, 743c, 744a, 744t, 756a, 760a, 763g, 765c, 765g, 768c, 770a, 770t, 771c, 771t, 772a, 772t, |
| | | | 774t, 777c, 780c, 780g, 785a, 785t, 791a, 792t, 795a, 795g, 795t, 799a, 799t, 800t, 801g, 802t, 804a, 804c, 807a, 807g, 809t, 810t, 811g, |
| | | | 813t, 814g, 816c, 819c, 822c, 823a, 824t, 825c, 826a, 828c, 831t, 832g, 833t, 834c, 834g, 834t, 837a, 838a, 838t, 840g, 840t, 842c, 843c, |
| | | | 843g, 846c, 847g, 847t, 848a, 849c, 852c, 855a, 855t, 858a, 859t, 861g, 861t, 864c, 867g, 869g, 870c, 873a, 876g, 876t, 878t, 879c, 880t, |
| | | | 882a, 885a, 885c, 886a, 888g, 894a, 894c, 894g, 897g, 900c, 906a, 906c, 906g, 912a, 912c, 915a, 918a, 918c, 920g, 921t, 923c, 924a, 924c, |
| | | | 926g, 927g, 928t, 930c, 930g, 930t, 933a, 933c, 933t, 936c, 936g, 936t, 939a, 940g, 942c, 942g, 945a, 951t, 952g, 954c, 957c, 958c, 960a, |
| | | | 963c, 966c, 966g, 969c, 970g, 972t, 973a, 974t, 975c, 978a, 978c, 978g, 981c, 983a, 983t, 984a, 987c, 987g, 987t, 988g, 990a, 994a, 996t, |
| | | | 997c, 999t, 1002c, 1002g, 1004a, 1005g, 1008t, 1010t, 1011a, 1011g, 1011t, 1014g, 1014t, 1016t, 1017g, 1017t, 1018t, 1020c, 1020g, |
| | | | 1021a, 1021t, 1023c, 1023g, 1023t, 1026a, 1026c, 1029g, 1029t, 1032t, 1035c, 1036a, 1038t, 1039a, 1041t, 1042t, 1044t, 1050c, 1056a, |
| | | | 1059a, 1059t, 1060g, 1065g, 1067t, 1068a, 1070t, 1071c, 1074a, 1074t, 1076a, 1076c, 1077c, 1077g, 1078a, 1079t, 1080c, 1083c, 1084g, |
| | | | 1086a, 1086c, 1086g, 1087c, 1089g, 1090g, 1092c, 1094t, 1095a, 1095c, 1098t, 1101a, 1101t, 1102c, 1103c, 1104c, 1107c, 1111c, 1114t, |
| | | | 1115c, 1116t, 1117a, 1118t, 1119g, 1119t, 1122g, 1122t, 1125a, 1125t, 1127c, 1128c, 1128g, 1128t, 1129a, 1131t, 1132t, 1134a, 1134c, |
| | | | 1135a, 1137c, 1143t, 1145t, 1146a, 1146t, 1149a, 1149c, 1152c, 1155c, 1156a, 1158c, 1159c, 1161t, 1162g, 1164t, 1165g, 1167g, 1170g, |
| | | | 1171a, 1172c, 1173c, 1174a, 1174c, 1174g, 1175g, 1176g, 1176t, 1177a, 1179t, 1181g, 1182g, 1185a, 1185c, 1186t, 1188g, 1188t, 1189c, |
| | | | 1190a, 1190t, 1192t, 1194a, 1194c, 1194g, 1197g, 1197t, 1198g, 1199g, 1200a, 1202t, 1203c, 1203g, 1203t, 1206c, 1206t, 1207a, 1208t, |
| | | | 1209g, 1209t, 1212c, 1212g, 1212t, 1213t, 1215a, 1215c, 1218t, 1219c, 1221t, 1224c, 1227g, 1230g, 1232t, 1233g, 1236g, 1237g, 1238c, |
| | | | 1239a, 1239c, 1241a, 1242g, 1242t, 1245c, 1245g, 1246g, 1248c, 1249a, 1251g, 1254t, 1259g, 1260a, 1263c, 1266a, 1266t, 1269t, 1271a, |
| | | | 1271c, 1272c, 1281g, 1281t, 1284t, 1287t, 1288g, 1289c, 1290a, 1290c, 1290g, 1291g, 1293a, 1293c, 1296g, 1299a, 1300c, 1302a, 1305a, |
| | | | 1305c, 1305g, 1307g, 1308g, 1309g, 1311g, 1311t, 1314t, 1317c, 1320a, 1320c, 1323a, 1326t, 1327c, 1329g, 1330a, 1332t, 1333c, 1335g, |
| | | | 1336c, 1338a, 1339a, 1339t, 1341a, 1341c, 1344c, 1346g, 1347a, 1350a, 1350t, 1353g, 1354c, 1357a, 1357t, 1359g, 1365g, 1366t, 1367t, |
| | | | 1368g, 1371t, 1374c, 1377t, 1379g, 1380a, 1381t, 1382c, 1383a, 1383t, 1385t, 1386a, 1386c, 1386g, 1387g, 1389t, 1394g, 1395g, 1399g, |
| | | | 1401t, 1402g, 1404a, 1407g, 1407t, 1410c, 1412g, 1413a, 1414t, 1416a, 1416g, 1416t, 1419a, 1419c, 1419t, 1422t, 1425c, 1428t, 1431t, |
| | | | 1434g, 1434t, 1437t, 1439c, 1440t, 1443c, 1449g, 1452c, 1459a, 1488c |
| 68 | sense_5p_SARS2 | 98 | 4t, 8t, 9t, 11t, 13c, 15t, 45t, 50t, 51c, 59t, 62t, 63g, 67t, 78g, 90t, 110c, 140t, 141t, 143t, 143a, 166c, 172t, 181t, 186t, 189t, 199t, 208t, 220c, |
| | | | 222t, 239t, 240t, 241t, 245a, 262c, 274t, 278t, 282t, 287g, 288a, 288t, 296a, 300t, 304a, 308a, 310t, 313a, 314c, 320a, 326t, 329t, 330g, |
| | | | 335a, 336t, 337g, 338a, 341t, 355c, 358t, 358a, 382g, 387t, 406t, 418t, 429t, 431t, 442t, 459t, 459a, 460t, 461a, 461t, 466g, 475t, 502c, |
| | | | 505t, 526t, 559g, 560t, 562t, 565t, 589g, 595g, 597t, 600a, 601t, 601Δ, 608c, 626Δ, 626a, 626c, 643t, 647t, 653a, 670g, 733c, 42_43ins_t, |
| | | | 42_43ins_g, 42_43ins_n |
| 109 | sense_5p_SARS2_N250 | 209 | 13c, 29t, 30t, 207t, 241t, 367t, 537t, 670g, 682g, 705g, 793t, 800t, 813c, 820c, 823a, 831g, 834t, 838g, 839t, 840c, 848t, 851t, 860g, 879t, |
| | | | 887a, 899c, 915g, 921g, 927g, 928g, 929t, 931c, 937g, 952t, 955c, 962a, 975t, 978t, 981t, 984a, 988t, 991a, 991t, 995a, 997t, 998c, 1012c, |
| | | | 1015g, 1025g, 1026a, 1034a, 1051c, 1057g, 1060t, 1063t, 1067t, 1071t, 1075c, 1077a, 1077t, 1085a, 1086t, 1089t, 1097a, 1099t, 1102a, |
| | | | 1102c, 1103c, 1109a, 1115a, 1115t, 1118t, 1119g, 1124a, 1126g, 1127a, 1130t, 1132t, 1144c, 1149a, 1163g, 1171g, 1172a, 1173t, 1176t, |
| | | | 1177c, 1182g, 1195t, 1200g, 1204c, 1207t, 1212t, 1215g, 1216c, 1218t, 1220t, 1223g, 1229a, 1231t, 1233t, 1248a, 1248t, 1249t, 1250a, |
| | | | 1264t, 1270c, 1279g, 1290c, 1294a, 1294t, 1315t, 1324t, 1333c, 1340a, 1349t, 1351t, 1367g, 1378g, 1382c, 1384g, 1386t, 1387c, 1389a, |
| | | | 1390t, 1391t, 1395c, 1397c, 1398a, 1404g, 1405g, 1408t, 1411c, 1415c, 1420g, 1432t, 1436t, 1442a, 1450t, 1459g, 1465g, 1474t, 1483t, |
| | | | 1484g, 1485c, 1485t, 1486c, 1487t, 1488a, 1489t, 1492c, 1495t, 1504t, 1507t, 1515a, 1517a, 1517t, 1526a, 1536a, 1538a, 1539t, 1543t, |
| | | | 1557g, 1558c, 1561t, 1579t, 1582t, 1585t, 1588g, 1597a, 1597t, 1600t, 1604t, 1606c, 1607a, 1610t, 1613t, 1614a, 1614t, 1620a, 1622a, |
| | | | 1623t, 1624c, 1627t, 1629g, 1631t, 1632g, 1633t, 1638c, 1649a, 1650t, 1652c, 1653t, 1657t, 1663t, 1664c, 1666t, 1671t, 1672g, 1675g, |
| | | | 1679c, 1679t, 1681c, 1682t, 1683g, 1684t, 1707t, 1711t, 1712t, 1728t |
| 128 | antis_3p_SARS1_short | 41 | 3t, 4a, 26a, 27g, 29a, 29Δ, 30g, 30a, 31c, 32a, 32c, 38t, 38Δ, 40Δ, 41t, 46a, 48Δ, 48t, 48a, 49a, 50t, 51c, 51t, 52Δ, 52a, 55t, 58t, 28_29ins_a, |
| | | | 29_30ins_g, 29_30ins_a, 30_31ins_a, 31_32ins_a, 40_41ins_g, 41_42ins_g, 41_42ins_c, 42_43ins_a, 42_43ins_t, 43_44ins_g, |
| | | | 44_45ins_c, 45_46ins_a, 48_49ins_a |
| 129 | sense_3p_SARS1 | 21 | 8c, 8t, 9a, 10Δ, 34a, 48t, 49t, 64t, 102c, 102t, 104t, 112t, 116a, 118g, 155g, 155t, 199c, 209t, 225t, 236c, 7_8ins_c |
| 137 | antis_5p_SARS1 | 93 | 97g, 98g, 99g, 100g, 101g, 102g, 108a, 109a, 110a, 111a, 112a, 113a, 113g, 134g, 135g, 136g, 137g, 138g, 139g, 178c, 179c, 180c, 181c, |
| | | | 182c, 183c, 183a, 215c, 216c, 217c, 217t, 218c, 218t, 219c, 219t, 220c, 220t, 221t, 222t, 229a, 230a, 231a, 231g, 232a, 232g, 233a, 233g, |
| | | | 234a, 234g, 235g, 236g, 236a, 269a, 270a, 271a, 272a, 273a, 274a, 284a, 285a, 286a, 287a, 288a, 289a, 290a, 299t, 300t, 301t, 302t, 303t, |
| | | | 304t, 324t, 325t, 325g, 326t, 326g, 327Δ, 327t, 327g, 328Δ, 328t, 328g, 329t, 329g, 330g, 330a, 331t, 331Δ, 983a, 990t, 1017a, 1020a, |
| | | | 329_330ins_g, 330_331ins_g |
| 145 | antis_3p_SARS2 | 146 | 21a, 37a, 38a, 42a, 52a, 60t, 66a, 70a, 82c, 87g, 91g, 94a, 97c, 98c, 104t, 105g, 107g, 111a, 112a, 113t, 115g, 116a, 118c, 120g, 124c, 135c, |
| | | | 151a, 153a, 162t, 169g, 178a, 187a, 208a, 208t, 212g, 223c, 232g, 238a, 252t, 253a, 254a, 254t, 269a, 271a, 273t, 279c, 282a, 284a, 286g, |
| | | | 287c, 290a, 295a, 298g, 302c, 307a, 320c, 325g, 326a, 329a, 330t, 331c, 339c, 353t, 358g, 370a, 372a, 375t, 376c, 378t, 383c, 384a, 387a, |
| | | | 387t, 393t, 399g, 400g, 400t, 403a, 405t, 413a, 416a, 417t, 425a, 425t, 427g, 431a, 435a, 439a, 442a, 445c, 451g, 468t, 476t, 477c, 487c, |
| | | | 490g, 504g, 505a, 507t, 511a, 511t, 514a, 518t, 521a, 524a, 527a, 540t, 547g, 550a, 565c, 571g, 573a, 574c, 575c, 581c, 587c, 603g, 615t, |
| | | | 623a, 642c, 651a, 654a, 662g, 663a, 664c, 668a, 671c, 679t, 682g, 689g, 702a, 709a, 797c, 820c, 832c, 965a, 1135a, 1261a, 1295a, 1427g, |
| | | | 1427t, 1429t, 1432t, 1472a, 1473a, 1489g |
| 148 | sense_5p_RSV | 165 | 4g, 4t, 4a, 5t, 5Δ, 6t, 8t, 9g, 11g, 12a, 12g, 13t, 13g, 14t, 14g, 15g, 16g, 16c, 17g, 17t, 18g, 19g, 20g, 21g, 22g, 22c, 23g, 24g, 24t, 25g, 25Δ, |
| | | | 26g, 26t, 27g, 27a, 28g, 28a, 29g, 29t, 30g, 30t, 30a, 31c, 31t, 31g, 31a, 32a, 32t, 33g, 33c, 33t, 34a, 34g, 34t, 35g, 35t, 36a, 36t, 36g, 37c, |
| | | | 37a, 37t, 37g, 38g, 38c, 38t, 39t, 39g, 40g, 40t, 41g, 41t, 42g, 42t, 42c, 42Δ, 43g, 43t, 44a, 44t, 44g, 45t, 45Δ, 45g, 46g, 46a, 47g, 48g, 48a, |
| | | | 49a, 49g, 50Δ, 61a, 62a, 62t, 62g, 64t, 64c, 68t, 68c, 69c, 69t, 70t, 70g, 71c, 71g, 71t, 71a, 72c, 72g, 72t, 73t, 73c, 73g, 74c, 74t, 74g, 75c, |
| | | | 75g, 75t, 76g, 76t, 76c, 76a, 76Δ, 77t, 77c, 77g, 78g, 78t, 78a, 79c, 79t, 79g, 79a, 80t, 80g, 80a, 81c, 81g, 81t, 81a, 82t, 82c, 82a, 82g, 83t, |
| | | | 83g, 83a, 84t, 84g, 85t, 85a, 85g, 86t, 86a, 86g, 87t, 87g, 88t, 88g, 89g, 90g, 91g, 92g |
| 149 | sense_5p_RSV_short | 4 | 4g, 29g, 29t, 30g |
| 154 | sense_3p_RSV | 383 | 3g, 4g, 4a, 4Δ, 5c, 5Δ, 5t, 6a, 6c, 6Δ, 7c, 7t, 8g, 8c, 8Δ, 8t, 9a, 9c, 10c, 14c, 15c, 15a, 16a, 16t, 16c, 17t, 17c, 18t, 18g, 19a, 19c, 19Δ, 20t, |
| | | | 20g, 20a, 20Δ, 21g, 21c, 21Δ, 21a, 22g, 22Δ, 22a, 23g, 23c, 23Δ, 23a, 23t, 24Δ, 24a, 24c, 25c, 25Δ, 26a, 26c, 26Δ, 27t, 27Δ, 28t, 28Δ, 28a, |
| | | | 29t, 29Δ, 29g, 30c, 30g, 30Δ, 30t, 31c, 31Δ, 32g, 32c, 32Δ, 33c, 33a, 34a, 36t, 36a, 37c, 37t, 38t, 39a, 39t, 42t, 42c, 42g, 43c, 44g, 44t, 44c, |
| | | | 44a, 45t, 45g, 46t, 47t, 47g, 48t, 48c, 49a, 49t, 50g, 50t, 50a, 51c, 51t, 51a, 52a, 52t, 52Δ, 53a, 53Δ, 53t, 54a, 54t, 55Δ, 55a, 55t, 56t, 56Δ, |
| | | | 56a, 57a, 57t, 57Δ, 58a, 58c, 59a, 59g, 60a, 61a, 61g, 62t, 63t, 63Δ, 63g, 64t, 64Δ, 65t, 65Δ, 65c, 66c, 66a, 68t, 68c, 70t, 71a, 71c, 72t, 72c, |
| | | | 73t, 73a, 73g, 73c, 74a, 74c, 76t, 77t, 77a, 78a, 79a, 80a, 80t, 80Δ, 81t, 81Δ, 81a, 81g, 81c, 82a, 82Δ, 82t, 83Δ, 83a, 83c, 84t, 84a, 85t, 85Δ, |
| | | | 85a, 86a, 86t, 86Δ, 86c, 87t, 88Δ, 88a, 88t, 88c, 88g, 89a, 89Δ, 89t, 89c, 90Δ, 90t, 90c, 90g, 90a, 91a, 91c, 92Δ, 92a, 93Δ, 93a, 94Δ, 94t, |
| | | | 94c, 94a, 97Δ, 98c, 99c, 99t, 100t, 100g, 100c, 101a, 101g, 102c, 102a, 102g, 103c, 103g, 103a, 104a, 104c, 104g, 105t, 105g, 106c, 106a, |
| | | | 107g, 107t, 107a, 108a, 109g, 109a, 110g, 110t, 110c, 110a, 111c, 111t, 111a, 111g, 112a, 113g, 113a, 116t, 116a, 117a, 117t, 117c, 117g, |
| | | | 118a, 118g, 119g, 120g, 122a, 123a, 123c, 124Δ, 124c, 125t, 126t, 126g, 127t, 127a, 128g, 128a, 128t, 129a, 129g, 129t, 130c, 130a, 130g, |
| | | | 131g, 131t, 131c, 132g, 132c, 133g, 133a, 133t, 133Δ, 134g, 134a, 134t, 134c, 135a, 135c, 137c, 137a, 137t, 138a, 138g, 140a, 140Δ, 141a, |
| | | | 141t, 142a, 142c, 144g, 145g, 146c, 147g, 147c, 149g, 149a, 150a, 151t, 151a, 152t, 152a, 154t, 154a, 155t, 155g, 155a, 156t, 156a, 156g, |
| | | | 158t, 159c, 159a, 162c, 163a, 163c, 165c, 165a, 168c, 169a, 171c, 174g, 174a, 175a, 176a, 179c, 179a, 180a, 182g, 182a, 185t, 185g, 187c, |
| | | | 187t, 188a, 188c, 192c, 5_6ins_a, 7_8ins_t, 7_8ins_g, 17_18ins_t, 19_20ins_a, 47_48ins_t, 49_50ins_t, 54_55ins_g, 56_57ins_t, |
| | | | 56_57ins_a, 59_60ins_a, 60_61ins_t, 60_61ins_a, 65_66ins_t, 65_66ins_a, 67_68ins_a, 67_68ins_t, 78_79ins_t, 81_82ins_t, 81_82ins_a, |
| | | | 83_84ins_t, 83_84ins_a, 84_85ins_t, 84_85ins_c, 84_85ins_a, 85_86ins_a, 86_87ins_t, 88_89ins_a, 88_89ins_t, 89_90ins_a, 89_90ins_t, |
| | | | 93_94ins_a, 93_94ins_t, 94_95ins_a, 94_95ins_t |
| 158 | antis_5p_RSV | 775 | 4g, 4t, 4c, 4a, 5g, 5a, 5t, 5c, 6t, 7c, 7g, 7t, 7a, 8g, 8a, 8t, 9g, 9t, 9c, 9a, 10g, 10t, 10a, 11t, 11a, 12t, 13g, 13t, 13a, 14g, 14c, 14t, 14a, 15c, |
| | | | 15g, 15a, 15t, 16g, 16t, 16a, 16c, 16Δ, 17g, 17t, 17Δ, 17c, 18g, 18c, 18t, 18a, 19t, 19a, 19c, 19Δ, 20g, 20t, 20c, 21g, 21t, 21c, 22g, 22a, 22t, |
| | | | 22c, 23g, 23t, 23a, 23c, 24g, 24a, 24t, 25t, 25g, 25c, 25a, 26t, 26c, 27t, 27Δ, 27a, 28t, 28g, 28a, 29t, 29a, 29c, 29g, 30t, 30g, 30a, 30c, 31a, |
| | | | 31c, 31t, 31g, 32t, 32a, 32c, 33g, 33t, 33c, 33a, 34t, 34c, 34Δ, 35t, 35a, 35c, 35g, 36t, 36a, 36g, 37g, 37t, 37a, 37Δ, 38g, 38t, 38Δ, 38c, 39c, |
| | | | 39Δ, 40t, 40a, 40g, 40c, 40Δ, 41t, 41a, 41c, 41Δ, 42t, 42c, 42a, 42g, 42Δ, 43g, 43a, 43c, 43Δ, 43t, 44c, 44g, 44a, 44t, 44Δ, 45g, 45c, 45a, |
| | | | 45t, 46t, 46a, 46c, 46g, 47g, 47t, 47c, 47a, 48t, 48c, 48Δ, 48a, 48g, 49a, 49t, 49Δ, 50a, 50t, 50g, 50c, 51g, 51a, 51t, 51c, 52t, 52a, 52g, 53a, |
| | | | 53t, 53g, 53c, 53Δ, 54t, 54g, 54a, 54c, 54Δ, 55g, 55t, 55c, 55a, 55Δ, 56a, 56t, 56c, 56Δ, 57g, 57t, 57a, 57c, 57Δ, 58a, 58c, 58t, 58g, 59c, 59a, |
| | | | 59t, 59Δ, 60a, 60t, 60c, 60g, 60Δ, 61t, 61c, 61Δ, 61g, 61a, 62t, 62g, 62c, 62Δ, 62a, 63c, 63t, 63Δ, 63a, 63g, 64c, 64t, 64g, 64Δ, 64a, 65c, 65t, |
| | | | 65a, 65Δ, 66t, 66c, 66a, 66Δ, 67t, 67c, 67a, 68t, 68g, 68a, 68Δ, 69g, 69t, 69c, 69a, 69Δ, 70g, 70t, 70c, 70a, 70Δ, 71t, 71a, 71c, 71Δ, 72t, 72c, |
| | | | 72g, 72Δ, 72a, 73c, 73t, 73a, 73g, 74t, 74c, 74a, 75g, 75c, 75t, 75a, 76t, 76c, 76a, 76g, 77t, 77c, 77a, 77g, 77Δ, 78t, 78c, 78Δ, 79c, 79g, 79a, |
| | | | 80c, 80a, 80t, 80Δ, 81c, 81t, 81a, 81g, 81Δ, 82c, 82g, 82t, 82a, 83c, 83t, 83a, 84t, 84Δ, 84a, 85g, 85Δ, 85t, 85a, 86t, 86c, 86Δ, 86a, 87t, 87c, |
| | | | 87g, 87a, 87Δ, 88c, 88t, 88a, 88g, 89g, 89a, 89t, 90c, 90t, 90Δ, 90g, 90a, 91t, 91c, 91Δ, 91a, 91g, 92t, 92a, 92c, 92Δ, 93t, 93Δ, 93g, 93a, 94t, |
| | | | 94g, 94Δ, 94a, 95Δ, 95g, 95a, 96c, 96g, 96a, 96t, 97g, 97c, 97a, 97Δ, 97t, 98Δ, 98a, 98t, 98g, 99c, 99Δ, 99a, 99g, 100g, 100t, 100Δ, 100a, |
| | | | 101g, 101c, 101t, 101a, 101Δ, 102g, 102Δ, 102t, 102a, 102c, 103t, 103Δ, 103a, 103g, 104t, 104a, 104c, 104Δ, 104g, 105t, 105c, 105g, 105a, |
| | | | 105Δ, 106a, 106t, 106c, 106g, 106Δ, 107c, 107a, 107Δ, 107t, 107g, 108t, 108Δ, 108g, 108a, 108c, 109t, 109c, 109Δ, 109g, 110t, 110Δ, |
| | | | 110a, 110g, 111t, 111Δ, 111a, 111g, 112t, 112g, 112Δ, 112a, 113g, 113t, 113Δ, 113a, 114t, 114g, 114Δ, 114a, 114c, 115a, 115Δ, 115t, |
| | | | 115g, 116a, 116t, 116Δ, 116g, 117t, 117Δ, 117a, 117g, 118t, 118a, 118Δ, 118g, 119t, 119Δ, 119a, 119c, 119g, 120a, 120t, 120Δ, 121a, |
| | | | 121Δ, 121t, 121g, 122Δ, 122a, 122t, 122g, 123t, 123Δ, 123a, 124t, 124Δ, 124a, 124g, 124c, 125t, 125Δ, 125a, 125g, 126a, 126t, 126Δ, |
| | | | 126g, 127Δ, 127t, 127a, 127g, 128t, 128a, 128Δ, 128c, 128g, 129t, 129a, 129Δ, 129g, 130t, 130a, 130Δ, 131a, 131Δ, 131t, 132t, 132a, 133t, |
| | | | 133a, 134t, 134Δ, 134a, 134g, 134c, 135a, 135Δ, 135c, 135g, 137a, 137t, 137c, 138t, 138a, 138g, 139a, 139t, 139g, 140a, 141a, 141g, 141t, |
| | | | 142a, 142g, 143g, 143a, 143Δ, 143t, 144a, 144Δ, 145a, 145Δ, 145t, 146a, 146t, 146Δ, 147a, 147g, 147t, 147Δ, 148a, 148g, 148c, 148Δ, |
| | | | 149g, 149t, 150g, 150c, 151g, 152t, 152c, 153t, 153a, 153g, 154g, 155g, 156c, 156g, 157t, 157g, 157c, 158t, 159a, 159t, 159g, 159c, 160a, |
| | | | 160g, 160c, 160t, 160Δ, 161t, 161g, 161Δ, 162c, 162t, 162a, 162g, 162Δ, 163a, 163g, 163t, 163Δ, 164g, 164a, 164t, 164Δ, 165a, 165t, |
| | | | 165g, 165Δ, 165c, 166g, 166a, 166Δ, 166t, 167g, 167t, 167a, 167Δ, 168a, 168g, 168c, 168Δ, 169a, 169c, 169g, 169t, 170a, 170g, 170t, |
| | | | 171g, 171t, 171a, 172c, 172a, 172g, 173g, 173c, 173a, 173Δ, 174g, 174a, 174c, 174Δ, 175g, 175t, 175Δ, 175a, 176g, 176c, 176Δ, 177a, |
| | | | 177g, 177t, 177Δ, 178g, 178a, 178Δ, 178t, 179g, 179t, 179Δ, 180Δ, 180a, 181c, 181a, 181t, 181Δ, 182g, 182a, 182c, 182t, 182Δ, 183a, |
| | | | 183c, 183g, 183t, 183Δ, 184c, 184g, 185g, 186g, 186Δ, 187a, 187g, 187Δ, 187c, 187t, 188a, 188Δ, 188t, 189c, 189Δ, 190a, 190t, 190Δ, |
| | | | 191t, 191Δ, 192a, 192g, 192Δ, 193c, 193t, 196a, 196t, 197a, 197t, 197c, 197g, 198a, 198c, 198g, 199t, 201t, 202g, 203c, 203g, 203Δ, 204g, |
| | | | 204c, 204t, 206t, 207t, 207Δ, 208c, 211g, 211c, 63_64ins_t, 73_74ins_a, 96_97ins_a, 96_97ins_t, 97_98ins_a, 97_98ins_t, 98_99ins_t, |
| | | | 98_99ins_a, 101_102ins_t, 102_103ins_t, 102_103ins_a, 103_104ins_t, 106_107ins_t, 110_111ins_t, 111_112ins_t, 111_112ins_a, |
| | | | 112_113ins_t, 112_113ins_a, 113_114ins_a, 115_116ins_a, 115_116ins_t, 117_118ins_t, 120_121ins_t, 120_121ins_a, 121_122ins_t, |
| | | | 121_122ins_a, 122_123ins_t, 122_123ins_a, 124_125ins_t, 125_126ins_g, 126_127ins_t, 126_127ins_a, 126_127ins_g, 127_128ins_t, |
| | | | 127_128ins_a, 137_138ins_t, 137_138ins_a, 148_149ins_t, 161_162ins_c, 161_162ins_a, 164_165ins_t, 166_167ins_t, 172_173ins_t, |
| | | | 176_177ins_t, 180_181ins_t, 184_185ins_c, 184_185ins_a, 185_186ins_t, 188_189ins_t, 195_196ins_t, 196_197ins_t, 198_199ins_a, |
| | | | 210_211ins_t |
| 165 | antis_5p_RSV_no_flank | 242 | 4t, 4c, 5t, 14g, 20g, 20t, 24g, 29a, 29t, 30t, 30a, 31c, 31t, 32t, 33g, 34t, 35a, 35t, 36a, 36t, 38g, 38t, 40c, 40g, 43Δ, 43t, 44c, 44a, 45c, 45g, |
| | | | 45a, 46t, 46a, 46Δ, 49t, 50a, 50t, 51a, 51t, 51c, 52t, 52g, 52Δ, 53t, 53a, 54t, 54a, 54c, 55g, 55t, 55c, 55a, 56a, 56t, 57g, 57t, 57c, 57a, 58c, |
| | | | 58a, 58t, 59c, 59a, 60a, 60c, 60t, 61c, 62c, 62t, 63Δ, 63c, 65c, 66t, 66c, 67c, 67t, 68t, 69g, 69t, 70g, 70t, 71a, 71t, 72t, 73c, 73t, 74t, 74c, |
| | | | 75g, 75c, 76t, 76c, 76a, 77a, 77t, 77c, 78t, 79c, 79g, 80a, 81c, 81a, 82c, 83c, 83a, 86c, 87g, 87t, 87a, 88a, 88t, 89t, 89a, 90t, 92t, 92a, 93t, |
| | | | 93g, 94t, 97a, 97g, 98g, 99a, 100a, 100g, 101g, 101t, 103t, 103g, 104t, 104g, 104Δ, 105t, 105a, 106a, 106t, 106Δ, 106g, 107Δ, 107a, 107g, |
| | | | 108t, 108g, 109t, 109g, 109Δ, 110t, 110Δ, 110g, 111g, 111Δ, 112g, 112t, 113g, 113t, 114t, 114g, 114a, 115a, 115g, 115Δ, 116a, 116t, |
| | | | 116Δ, 117t, 117Δ, 118t, 118Δ, 118a, 119t, 119a, 120a, 120t, 120Δ, 121Δ, 121a, 121t, 122a, 122Δ, 122t, 122g, 123t, 123a, 124Δ, 124t, 124a, |
| | | | 125t, 125Δ, 125a, 126a, 126Δ, 126t, 127Δ, 127t, 127a, 128t, 128a, 128c, 129a, 129t, 129Δ, 130t, 130a, 130Δ, 131a, 131Δ, 132t, 132a, 133t, |
| | | | 133a, 134t, 134Δ, 134a, 135Δ, 135a, 137a, 137c, 138a, 139a, 139g, 141a, 141t, 142a, 143a, 144a, 146a, 147a, 148a, 98_99ins_t, |
| | | | 98_99ins_a, 103_104ins_t, 112_113ins_t, 112_113ins_a, 115_116ins_a, 115_116ins_t, 117_118ins_t, 122_123ins_t, 122_123ins_a, |
| | | | 124_125ins_t, 125_126ins_g, 127_128ins_t, 148_149ins_t |
| 169 | antis_3p_RSV | 56 | 4c, 4a, 4Δ, 5c, 5t, 7t, 8a, 8g, 9a, 9t, 10a, 11a, 11t, 12g, 13a, 16g, 16a, 17g, 18g, 18t, 19c, 21c, 21a, 22g, 22t, 24a, 24t, 35t, 36t, 38g, 39c, 40a, |
| | | | 40c, 47Δ, 48t, 48a, 51t, 53c, 54c, 57c, 58c, 58a, 60g, 60a, 60Δ, 62c, 66c, 67c, 67a, 70t, 73a, 78a, 83c, 85t, 93c, 60_61ins_t |
| 170 | antis_3p_RSV_36TrC | 59 | 5c, 5t, 5a, 7c, 7a, 9a, 9g, 9t, 9c, 10a, 10c, 11a, 12a, 12g, 13a, 13g, 14c, 14g, 15a, 16g, 17g, 17c, 18g, 18t, 19c, 20a, 21g, 21c, 21a, 22t, 22g, |
| | | | 22c, 24a, 24t, 24c, 25c, 26g, 26a, 27a, 28c, 29g, 32c, 32t, 33g, 35t, 36g, 36t, 38g, 39c, 40a, 40c, 41a, 45Δ, 45c, 48t, 48a, 54c, 58c, 60g |
| 177 | antis_3p_RSV_short | 7 | 11c, 15g, 15c, 22c, 24c, 25a, 50c |
| 179 | sense_3p_HPIV1_FR2011 | 92 | 7a, 13a, 13g, 21a, 29t, 30g, 30a, 31t, 31Δ, 32g, 32a, 32Δ, 34c, 37t, 37a, 37Δ, 37c, 38a, 41a, 41c, 42c, 43a, 43Δ, 45t, 45Δ, 46a, 46t, 47t, 47a, |
| | | | 48a, 48t, 50a, 50t, 50c, 51a, 51c, 52a, 52Δ, 53a, 53c, 54c, 55a, 55c, 57c, 57a, 60a, 64Δ, 64c, 67t, 67Δ, 69Δ, 70a, 70t, 71Δ, 71a, 72a, 72Δ, |
| | | | 72c, 78a, 79t, 79Δ, 80t, 80a, 80Δ, 81t, 81Δ, 81a, 82a, 82c, 84t, 84c, 85c, 86t, 86c, 87c, 87a, 89Δ, 89c, 89t, 94Δ, 94t, 94c, 95Δ, 95a, 99c, 99Δ, |
| | | | 101Δ, 101t, 102a, 106c, 107t, 1074 |
| 180 | sense_5p_HPIV1_FR2011 | 31 | 12a, 14a, 14g, 16a, 17t, 17Δ, 26t, 32a, 40a, 42a, 42t, 45a, 47a, 52t, 58t, 58a, 60a, 64c, 68c, 68Δ, 74c, 76c, 77a, 91t, 102c, 103a, 105a, 108a, |
| | | | 109c, 124a, 63_64ins_c |
| 181 | antis_5p_HPIV1_FR2011 | 137 | 29t, 32g, 32t, 34a, 34t, 34g, 35t, 35g, 36t, 36a, 38t, 39g, 40t, 40g, 41g, 41t, 42t, 45g, 45t, 46t, 46a, 47t, 48g, 48t, 49g, 49t, 50t, 50a, 51t, 51a, |
| | | | 52g, 52a, 52t, 53t, 53g, 55t, 56g, 56t, 59a, 60a, 60t, 62a, 63a, 63g, 64t, 64a, 65t, 66t, 67a, 67g, 69g, 73t, 73g, 74t, 74g, 75g, 76g, 77t, 77g, |
| | | | 79g, 79a, 80g, 81t, 81g, 82g, 82t, 83t, 84g, 85a, 86g, 86Δ, 87g, 87Δ, 90g, 90t, 90a, 91t, 91a, 94t, 94g, 95t, 96t, 96a, 97a, 97t, 98t, 98a, 99a, |
| | | | 99t, 101t, 102a, 103t, 103a, 105t, 107t, 108t, 109a, 109t, 110t, 111t, 111a, 115a, 115t, 116t, 116a, 117a, 118t, 120a, 121t, 122t, 123t, 124a, |
| | | | 125a, 125t, 126t, 126a, 127t, 128a, 128t, 129t, 129a, 132a, 133a, 134t, 134a, 136t, 137Δ, 137a, 137t, 138t, 138a, 140c, 140t, 141a, 141t, |
| | | | 142t, 144t |
| 184 | antis_3p_HPIV1_FR2011 | 13 | 5Δ, 7g, 8g, 12t, 13a, 34t, 37c, 37a, 42t, 45g, 74c, 99t, 107t |
| 185 | sense_3p_HPIV3_Wash | 134 | 3a, 4a, 4t, 4g, 6a, 7a, 7c, 7g, 8a, 8t, 8g, 9c, 9a, 9Δ, 10a, 10t, 10g, 11a, 12a, 14a, 14c, 16a, 16g, 17a, 17c, 19c, 19a, 20c, 22c, 22a, 23c, 24c, |
| | | | 25c, 25a, 28g, 28t, 29g, 29c, 30g, 30t, 30c, 31g, 31c, 32g, 32c, 33c, 33g, 34g, 34c, 35c, 37c, 37g, 38g, 38c, 39c, 39g, 40g, 40a, 40c, 42c, |
| | | | 42g, 42a, 43c, 43a, 43g, 44c, 44a, 44g, 45t, 45g, 45c, 46c, 47a, 47c, 48g, 48c, 49g, 49c, 50g, 50c, 50t, 51c, 53g, 53c, 54g, 54c, 55g, 55c, |
| | | | 56c, 57g, 57c, 59g, 59c, 60g, 60c, 61c, 64g, 64c, 65c, 66g, 66c, 68g, 69t, 69c, 70c, 71g, 71c, 72c, 74g, 74c, 76g, 76t, 76c, 77c, 78c, 79c, |
| | | | 80c, 81c, 82c, 82t, 84g, 84c, 85g, 85c, 86c, 86t, 87c, 89c, 90a, 90c, 92c, 93g, 93c, 100c |
| 186 | sense_5p_HPIV3_Wash | 84 | 4t, 4c, 4g, 5t, 6g, 6a, 9t, 9c, 9g, 10g, 11g, 12g, 12t, 13c, 13a, 13g, 14t, 14g, 15a, 15c, 16g, 21c, 21g, 22g, 22c, 23g, 23c, 23a, 24g, 24a, 25g, |
| | | | 25t, 27c, 27t, 28c, 28t, 29c, 29t, 31c, 33c, 33a, 34c, 34a, 35c, 35a, 36a, 36c, 37c, 37a, 37g, 38a, 39g, 39t, 40t, 40a, 41t, 42t, 43t, 44a, 44t, |
| | | | 45g, 45a, 45t, 46a, 46t, 47a, 47c, 49a, 49t, 51t, 51a, 53t, 54g, 57a, 58g, 74c, 88a, 89a, 90a, 72_73ins_a, 72_73ins_t, 90_91ins_a, |
| | | | 91_92ins_a, 92_93ins_a |
| 187 | antis_5p_HPIV3_Wash | 172 | 4t, 4c, 6t, 9t, 12t, 13t, 15t, 15a, 16t, 20t, 23t, 31a, 31t, 32a, 33a, 33c, 34c, 34a, 35a, 36a, 36g, 36t, 37a, 37g, 38t, 38g, 38a, 39g, 39a, 39t, 41a, |
| | | | 41g, 41t, 42a, 42t, 43a, 43t, 43c, 44a, 44t, 44g, 45a, 45g, 46a, 46t, 46g, 47a, 47g, 47t, 48t, 48g, 48a, 48c, 49a, 49g, 49t, 49c, 50t, 50a, 50g, |
| | | | 50c, 51a, 51t, 51g, 51c, 52a, 52t, 52g, 53a, 53g, 53t, 54g, 54t, 54c, 55c, 55g, 56a, 56t, 57a, 57t, 57g, 57c, 58t, 58c, 58g, 58a, 59a, 59g, 59t, |
| | | | 60a, 60g, 60c, 61a, 61t, 61g, 61c, 62a, 62t, 62g, 62c, 63a, 63c, 63g, 64t, 64c, 64a, 65t, 65g, 65c, 66t, 66g, 66a, 66c, 67t, 67a, 67g, 68c, 68t, |
| | | | 68g, 68a, 69g, 69a, 69t, 69c, 70a, 70g, 70c, 71c, 71t, 71a, 72a, 72c, 72t, 72g, 73g, 73c, 74g, 74c, 75t, 75g, 75c, 76g, 76c, 77g, 77c, 78g, 78t, |
| | | | 78c, 78a, 79g, 81c, 83c, 84g, 84c, 88c, 89c, 90c, 90g, 91c, 92c, 93c, 96c, 96g, 97c, 100c, 101c, 107g, 107c, 108a, 3_4ins_t, 38_39ins_a, |
| | | | 38_39ins_t |
| 190 | antis_3p_HPIV3_Wash | 31 | 5a, 6g, 7g, 10g, 11g, 16a, 16c, 16t, 17c, 18c, 22c, 22a, 23t, 28g, 33c, 36c, 38c, 44t, 66a, 67g, 68g, 69g, 81c, 83c, 83a, 85t, 86g, 86t, 87g, |
| | | | 88g, 94t |
| 192 | sense_3p_HMPV_NL | 1 | 41a |
| 193 | sense_5p_AMPV | 32 | 13a, 19a, 22g, 60a, 63a, 65g, 68a, 83g, 94a, 96t, 97g, 103a, 110a, 117t, 119g, 126g, 128g, 132t, 137a, 146g, 147t, 153t, 157g, 158c, 175g, |
| | | | 181t, 182c, 197a, 197Δ, 204a, 205g, 207a |
| 194 | sense_5p_HMPV_NL | 44 | 9t, 10a, 16a, 19c, 23a, 23t, 28g, 30c, 31t, 31a, 31g, 37g, 37t, 37c, 38t, 38a, 40a, 40c, 51t, 51g, 52a, 52c, 58t, 63g, 65a, 75t, 75a, 76g, 82a, |
| | | | 93a, 94t, 116a, 116g, 123Δ, 123c, 125a, 128g, 133t, 139c, 78_79ins_a, 82_83ins_a, 89_90ins_a, 90_91ins_t, 149_150ins_t |
| 195 | antis_5p_AMPV | 48 | 6Δ, 6t, 10Δ, 10a, 35t, 37c, 38t, 38c, 45t, 45g, 46c, 50a, 56a, 57c, 60g, 61a, 66t, 67c, 71a, 75c, 85c, 86a, 89a, 93t, 100t, 105t, 106c, 107a, |
| | | | 109t, 110a, 110c, 114g, 114t, 116c, 120a, 120c, 123c, 132t, 135t, 138c, 140t, 143t, 146a, 148t, 174t, 181c, 184t, 190t |
| 196 | antis_5p_HMPV_NL | 89 | 4g, 4t, 8c, 9c, 12c, 14a, 14c, 15a, 17c, 17a, 20a, 20g, 21c, 22a, 22t, 24t, 27c, 28a, 31a, 32Δ, 32t, 34a, 34c, 37a, 46a, 49a, 49t, 55a, 55g, 57a, |
| | | | 57g, 59g, 59a, 60a, 60g, 61a, 65a, 66t, 66c, 71a, 71c, 74a, 74c, 75t, 79t, 79c, 81a, 82a, 85a, 88t, 88c, 89a, 91c, 91a, 92a, 92g, 98a, 98t, 100c, |
| | | | 102a, 102t, 103t, 103c, 104a, 104c, 106c, 106t, 112c, 112t, 115t, 117a, 118c, 118t, 121t, 124a, 124t, 130t, 133a, 133t, 134g, 134c, 136g, |
| | | | 136c, 139t, 145t, 5_6ins_a, 6_7ins_a, 7_8ins_t, 52_53ins_t |
| 201 | antis_3p_HMPV_NL | 1 | 7t |
| 202 | sense_3p_NiV_UMMC1 | 21 | 5a, 6a, 7a, 8a, 8c, 10a, 10c, 14a, 14c, 23c, 25a, 26c, 26a, 26g, 33c, 33g, 38c, 39c, 40c, 41c, 47c |
| 203 | sense_5p_NiV_UMMC1 | 30 | 8g, 10g, 13g, 14a, 15g, 16a, 18g, 20g, 21c, 23c, 24c, 26c, 27t, 29t, 30g, 32g, 32Δ, 33c, 34c, 34a, 35a, 36g, 38g, 39c, 41c, 42a, 43g, 43a, 44a, |
| | | | 29_30ins_c |
| 204 | antis_5p_NiV_UMMC1 | 115 | 19g, 19Δ, 37t, 39c, 39g, 40g, 40t, 41t, 41g, 42t, 42c, 43t, 43c, 43g, 44t, 44c, 44g, 45t, 45c, 45g, 46g, 46c, 47t, 47g, 47c, 48t, 48g, 48c, 49a, |
| | | | 49c, 49g, 50g, 50c, 51g, 51c, 51t, 51a, 52t, 52g, 52a, 52c, 53a, 53t, 53c, 53g, 54c, 54g, 55g, 55a, 56g, 56c, 56a, 57g, 57c, 57a, 58c, 58a, |
| | | | 58g, 59c, 59a, 59g, 60a, 60g, 60c, 61g, 61c, 62g, 62t, 62a, 62c, 63a, 63g, 64g, 64c, 64t, 65c, 65g, 65t, 66g, 66c, 66t, 67c, 67a, 68a, 68g, 68c, |
| | | | 68t, 69g, 69c, 69t, 70g, 70t, 70c, 71g, 71c, 71t, 72c, 72t, 73g, 73t, 73c, 74g, 74c, 75t, 75c, 76c, 76t, 77c, 78c, 79c, 81t, 81c, 82c, 87c, |
| | | | 21_22ins_g |
| 206 | antis_3p_NiV_UMMC1 | 4 | 32c, 33t, 66g, 99t |
| 207 | sense_5p_HeV_AUS | 12 | 3g, 3a, 6t, 23g, 25a, 30Δ, 36Δ, 45c, 32_33ins_t, 33_34ins_t, 38_39ins_t, 39_40ins_t |
| 208 | sense_3p_HeV_AUS | 10 | 4a, 41t, 43g, 47c, 73t, 93t, 96c, 98a, 109g, 106_107ins_g |
| 209 | antis_5p_HeV_AUS | 35 | 10Δ, 11Δ, 12Δ, 13Δ, 15Δ, 21Δ, 40a, 41a, 41t, 42a, 42t, 43a, 43t, 43c, 44t, 44c, 45a, 45t, 45c, 46a, 46t, 48t, 51a, 54g, 54c, 54t, 74t, 76c, 96c, |
| | | | 6_7ins_a, 7_8ins_a, 8_9ins_a, 9_10ins_a, 11_12ins_a, 18_19ins_a |
| 211 | antis_3p_HeV_AUS | 10 | 4c, 15t, 17g, 20a, 40a, 66g, 70c, 72a, 109t, 6_7ins_c |
| 227 | antis_5p_ZEBOV | 2929 | 4Δ, 4a, 4g, 4t, 4c, 5t, 5g, 5c, 5a, 6g, 6a, 6t, 6c, 7t, 7c, 7a, 7g, 8t, 8c, 8a, 8g, 9Δ, 9c, 9g, 9a, 10Δ, 10t, 10g, 10a, 10c, 11c, 11t, 11a, 11g, 12g, |
| | | | 12t, 12c, 13t, 13g, 13a, 13c, 14t, 14c, 14a, 14g, 15a, 15g, 15c, 15t, 16t, 16g, 16a, 16c, 17g, 17t, 17a, 17c, 18t, 18a, 18c, 18g, 19c, 19t, 19g, |
| | | | 19a, 20g, 20a, 20c, 21a, 21g, 21c, 22c, 22a, 22t, 22g, 23a, 23g, 23c, 23t, 24c, 24a, 24g, 24t, 25t, 25c, 25a, 25g, 26g, 26t, 26a, 26c, 27a, 27g, |
| | | | 27c, 28t, 28g, 28a, 28c, 29a, 29g, 29t, 30c, 30a, 30t, 30g, 31a, 31t, 31g, 31c, 32t, 32a, 32c, 32Δ, 32g, 33Δ, 33c, 33t, 33a, 33g, 34c, 34t, 34a, |
| | | | 34g, 35c, 35a, 35t, 36t, 36g, 36a, 36c, 37t, 37g, 37a, 37c, 38g, 38t, 38a, 38c, 39t, 39g, 39a, 39c, 40Δ, 40t, 40a, 40g, 40c, 41c, 41a, 41g, 41t, |
| | | | 41Δ, 42t, 42c, 42a, 42g, 42Δ, 43t, 43a, 43g, 43c, 44t, 44c, 44g, 44a, 45t, 45a, 45g, 45c, 46t, 46a, 46c, 46g, 47t, 47a, 47c, 47g, 48t, 48a, 48c, |
| | | | 48g, 48Δ, 49c, 49g, 49t, 49a, 50t, 50g, 50a, 50c, 51g, 51a, 51c, 52t, 52g, 52a, 52c, 53t, 53a, 53g, 53c, 54a, 54c, 54g, 54t, 55t, 55c, 55g, 55a, |
| | | | 56c, 56g, 56t, 56a, 57g, 57a, 57t, 57c, 58g, 58a, 58c, 58Δ, 59t, 59g, 59a, 59c, 60t, 60a, 60g, 60c, 61t, 61a, 61c, 61g, 62t, 62c, 62g, 62a, 63t, |
| | | | 63a, 63g, 63c, 64g, 64t, 64a, 64c, 65a, 65t, 65c, 65g, 66t, 66a, 66c, 66g, 67t, 67c, 67a, 67g, 68t, 68a, 68c, 68g, 69c, 69t, 69a, 69g, 70t, 70a, |
| | | | 70c, 71t, 71a, 71c, 71g, 72t, 72a, 72c, 72g, 73t, 73a, 73c, 73g, 74t, 74a, 74c, 74g, 74Δ, 75t, 75a, 75c, 75g, 76t, 76a, 76c, 76g, 77t, 77a, 77c, |
| | | | 77g, 78t, 78a, 78c, 78g, 79t, 79a, 79c, 79g, 80a, 80t, 80c, 80g, 81c, 81t, 81a, 82t, 82a, 82c, 82g, 83a, 83t, 83c, 83g, 84t, 84a, 84c, 84g, 85c, |
| | | | 85t, 85a, 85g, 86t, 86c, 86a, 86g, 87t, 87a, 87c, 87g, 88a, 88t, 88c, 89t, 89a, 89c, 89g, 90t, 90a, 90c, 90g, 91t, 91a, 91c, 91g, 92a, 92c, 92g, |
| | | | 93c, 93a, 93t, 93g, 94a, 94c, 94t, 94g, 95a, 95t, 95c, 95g, 96a, 96c, 96t, 97a, 97c, 97t, 98c, 98a, 98t, 98g, 99a, 99c, 99t, 100a, 100c, 100g, |
| | | | 101a, 101c, 101t, 101g, 102a, 102c, 102t, 102g, 103a, 103c, 103t, 103g, 104a, 104c, 104t, 105c, 105t, 105g, 105a, 106c, 106g, 106a, 107c, |
| | | | 107a, 107g, 107Δ, 108c, 108a, 108g, 108t, 109c, 109a, 109g, 110c, 110a, 110g, 111c, 111a, 111g, 112c, 112a, 112g, 113a, 113c, 113g, |
| | | | 113t, 114c, 114a, 114g, 114t, 115c, 115a, 115t, 115g, 116c, 116a, 116t, 116g, 117c, 117a, 117t, 117g, 118a, 118c, 118t, 118g, 119a, 119c, |
| | | | 119g, 119t, 120a, 120t, 120g, 120c, 121a, 121t, 121c, 121g, 122a, 122g, 122c, 122t, 123t, 123a, 123g, 124a, 124t, 124g, 124c, 125a, 126a, |
| | | | 126g, 126Δ, 127a, 127t, 127g, 127c, 127Δ, 128a, 128g, 128t, 128c, 129a, 129t, 129g, 129c, 130g, 130t, 130c, 130a, 131a, 131t, 131g, 131c, |
| | | | 132a, 132t, 132g, 133a, 133t, 133g, 134a, 134t, 134g, 134c, 135a, 135t, 135c, 135g, 136a, 136t, 136c, 136g, 137a, 137t, 137c, 137g, 138a, |
| | | | 138t, 138c, 138g, 139a, 139g, 139t, 139c, 139Δ, 140a, 140g, 140t, 140c, 141a, 141t, 141c, 141g, 142c, 142a, 142t, 142g, 143a, 143g, 143t, |
| | | | 143c, 144a, 144g, 144t, 145a, 145g, 145t, 145c, 146a, 146g, 146t, 146c, 147a, 147t, 147c, 147Δ, 147g, 148a, 148t, 148c, 148g, 149a, 149t, |
| | | | 149c, 149g, 150a, 150t, 150c, 150g, 151a, 151t, 151c, 151g, 152a, 152t, 152c, 152g, 153a, 153g, 153t, 153c, 154a, 154t, 154g, 154c, 155a, |
| | | | 155g, 155t, 155c, 156a, 156c, 156t, 156g, 157a, 157t, 157g, 157c, 158a, 158t, 158g, 158c, 159g, 159a, 159t, 160a, 160t, 160g, 160c, 160Δ, |
| | | | 161a, 161t, 161g, 161Δ, 162c, 162a, 162t, 162g, 163a, 163c, 163g, 163t, 163Δ, 164a, 164t, 164g, 164c, 165a, 165t, 165g, 165c, 166a, 166g, |
| | | | 166c, 167g, 167a, 167t, 167c, 168a, 168t, 168g, 168c, 169g, 169a, 169t, 169c, 170a, 170t, 170c, 170g, 171a, 171t, 171c, 171g, 171Δ, 172t, |
| | | | 172a, 172g, 172c, 173a, 173g, 173c, 174a, 174t, 174g, 174c, 175a, 175t, 175g, 175c, 176a, 176t, 176c, 176g, 177a, 177t, 177g, 177c, 178a, |
| | | | 178t, 178g, 178c, 179a, 179t, 179g, 179c, 180a, 180t, 180g, 180c, 181a, 181t, 181g, 181c, 182a, 182t, 182c, 182g, 183a, 183t, 183g, 183c, |
| | | | 184a, 184t, 184g, 184c, 185a, 185t, 185Δ, 185c, 185g, 186a, 186t, 186c, 186g, 187a, 187t, 187c, 187g, 188t, 188a, 188g, 188c, 189a, 189t, |
| | | | 189g, 189c, 190a, 190t, 190g, 190c, 191a, 191t, 191g, 191c, 192a, 192t, 192g, 192c, 193a, 193t, 193c, 193g, 194a, 194t, 194c, 194g, 195a, |
| | | | 195g, 195t, 195Δ, 195c, 196t, 196c, 196a, 196g, 197a, 197c, 197g, 197Δ, 198c, 198a, 198g, 199c, 199g, 199a, 199t, 200t, 200c, 200a, 200g, |
| | | | 200Δ, 201t, 201c, 201g, 201a, 201Δ, 202a, 202g, 202t, 202c, 203a, 203g, 203t, 203c, 203Δ, 204a, 204g, 204c, 205a, 205g, 206c, 206a, |
| | | | 206g, 206t, 207t, 207c, 207a, 207g, 207Δ, 208t, 208a, 208g, 208c, 208Δ, 209a, 209g, 209c, 209t, 210t, 210a, 210g, 210c, 211t, 211a, 211g, |
| | | | 212a, 212t, 212g, 213a, 213t, 213c, 213g, 214a, 214c, 214t, 214g, 215a, 215t, 215g, 215c, 216g, 216c, 216a, 216t, 217a, 217c, 217t, 217g, |
| | | | 218t, 218c, 218a, 218g, 219a, 219t, 219c, 219g, 219Δ, 220t, 220c, 220a, 220g, 220Δ, 221t, 221c, 221a, 221g, 221Δ, 222t, 222a, 222g, 222c, |
| | | | 223t, 223c, 223g, 224a, 224t, 224c, 224g, 225t, 225c, 225a, 225g, 226a, 226t, 226c, 226g, 227t, 227c, 227a, 227g, 228t, 228c, 228a, 228Δ, |
| | | | 229a, 229t, 229c, 229g, 230a, 230t, 230c, 230g, 231a, 231t, 231c, 231g, 232a, 232t, 232c, 232g, 233a, 233c, 233t, 233g, 234a, 234t, 234c, |
| | | | 234Δ, 234g, 235a, 235t, 235c, 235g, 236a, 236t, 236c, 236g, 237a, 237t, 237c, 237g, 238t, 238c, 238a, 238g, 239t, 239c, 239g, 239a, 240a, |
| | | | 240c, 240g, 240t, 241a, 241c, 241g, 241t, 242a, 242c, 242g, 242t, 243a, 243c, 243g, 243t, 244c, 244a, 244g, 245a, 245c, 245g, 245t, 246a, |
| | | | 246g, 247a, 247g, 247t, 247c, 248a, 248g, 248t, 248c, 249Δ, 249a, 249g, 249t, 249c, 250Δ, 250a, 250g, 250c, 250t, 251a, 251g, 251t, 251c, |
| | | | 252a, 252g, 252t, 252c, 253a, 253g, 253t, 253c, 254a, 254g, 254t, 254c, 255a, 255g, 255t, 255c, 256a, 256g, 256t, 256c, 257g, 257a, 257t, |
| | | | 257c, 258a, 258g, 258t, 258c, 259a, 259g, 259t, 259c, 260g, 260t, 260a, 260c, 261g, 261a, 261t, 261c, 262g, 262t, 262a, 262c, 263g, 263a, |
| | | | 263t, 263c, 264g, 264t, 264a, 264c, 265g, 265t, 265c, 265a, 266g, 266a, 266c, 266t, 267a, 267g, 267t, 267c, 268c, 268g, 268a, 268t, 269c, |
| | | | 269t, 269a, 269g, 270c, 270a, 270t, 270g, 271c, 271t, 271a, 271g, 272t, 272c, 272g, 272a, 273a, 273c, 273t, 273g, 274a, 274t, 274c, 274g, |
| | | | 275t, 275c, 275g, 275a, 276t, 276a, 276c, 276g, 277t, 277a, 277c, 277g, 278a, 278t, 278c, 278g, 279a, 279t, 279c, 279g, 280a, 280t, 280c, |
| | | | 280g, 281g, 281a, 281t, 281c, 281Δ, 282t, 282c, 282a, 283a, 283t, 283c, 283g, 284a, 284g, 284c, 285a, 285t, 285c, 285g, 286t, 286a, 286c, |
| | | | 286g, 287g, 287a, 287t, 287c, 288t, 288a, 288c, 288g, 289a, 289t, 289c, 289g, 290a, 290t, 290c, 290g, 291c, 291a, 291g, 292t, 292a, 292g, |
| | | | 292c, 293t, 293a, 293g, 293c, 294c, 294t, 294a, 294Δ, 294g, 295c, 295t, 295a, 295g, 296t, 296a, 296c, 297a, 297t, 297c, 297g, 297Δ, 298t, |
| | | | 298a, 298c, 298g, 298Δ, 299t, 299a, 299c, 299g, 300t, 300a, 300c, 300g, 301t, 301a, 301c, 301g, 302a, 302c, 302t, 302g, 303t, 303a, 303g, |
| | | | 303c, 304a, 304g, 304t, 304c, 305a, 305t, 305g, 305c, 306t, 306c, 306g, 306a, 307g, 307t, 307c, 307a, 308c, 308t, 308g, 308a, 309c, 309g, |
| | | | 309t, 309a, 310c, 310t, 310g, 310a, 311t, 311c, 311g, 311a, 312g, 312t, 312c, 312a, 313t, 313g, 313c, 313a, 314t, 314g, 314c, 314a, 315g, |
| | | | 315c, 315a, 316g, 316c, 316a, 317c, 317t, 317g, 317a, 318t, 318g, 318c, 318a, 319g, 319c, 319t, 320g, 320c, 320t, 320a, 321g, 321c, 321a, |
| | | | 321t, 322g, 322c, 322a, 323g, 323c, 323a, 323t, 324g, 324c, 324a, 325g, 325c, 325t, 325a, 326g, 326c, 326a, 326t, 327g, 327c, 327a, 328g, |
| | | | 328c, 328a, 329g, 329c, 330g, 330c, 330a, 331g, 331c, 331a, 331t, 332c, 332g, 332t, 333c, 333g, 334c, 334g, 334a, 334t, 335g, 335c, 335a, |
| | | | 336c, 336g, 336a, 337Δ, 337c, 337g, 337a, 337t, 338c, 338g, 338a, 339c, 339t, 339g, 339a, 340g, 340c, 340a, 341c, 341g, 341a, 341t, |
| | | | 342Δ, 342c, 342g, 342a, 343Δ, 343c, 343g, 343a, 344c, 344g, 344a, 345Δ, 345g, 345a, 345c, 346Δ, 346g, 346c, 346a, 347g, 347c, 347t, |
| | | | 348g, 348a, 348c, 349c, 349g, 349a, 349t, 350c, 350g, 350a, 350t, 351g, 351a, 351t, 352c, 352g, 352a, 353c, 353g, 353a, 354c, 354g, 354a, |
| | | | 354t, 355c, 355g, 355a, 355t, 356g, 356c, 356a, 357c, 357g, 357a, 357Δ, 358g, 358a, 358c, 358Δ, 359c, 359g, 359a, 360t, 360c, 360g, |
| | | | 360a, 361c, 361g, 361t, 361a, 362c, 362g, 362a, 362t, 363c, 363g, 363a, 363t, 364c, 364g, 364a, 365c, 365g, 365a, 365t, 366c, 366g, 366a, |
| | | | 367t, 367c, 367g, 367a, 368t, 368c, 368g, 368a, 369c, 369g, 369a, 369t, 370t, 370c, 370g, 370a, 371c, 371g, 371a, 371t, 372c, 372t, 372g, |
| | | | 372a, 373c, 373g, 373a, 373t, 374c, 374g, 374a, 375c, 375t, 375g, 375a, 376t, 376c, 376a, 376g, 377c, 377g, 377a, 378c, 378g, 378a, 379c, |
| | | | 379t, 379g, 379a, 380c, 380g, 380a, 381c, 381g, 381a, 382g, 382c, 382a, 383t, 383g, 383c, 383a, 384t, 384g, 384a, 384c, 385t, 385g, 385c, |
| | | | 385a, 386g, 386a, 386c, 387t, 387g, 387c, 387a, 388t, 388g, 388c, 388a, 389t, 389g, 389c, 389a, 390g, 390c, 390a, 390t, 391g, 391a, 391c, |
| | | | 392t, 392g, 392c, 392a, 393t, 393g, 393c, 393a, 394t, 394g, 394a, 394c, 395g, 395c, 395a, 396g, 396c, 396a, 396Δ, 397g, 397a, 397c, 398g, |
| | | | 398a, 398c, 398t, 399c, 399g, 399a, 400t, 400c, 400g, 400a, 401t, 401g, 401c, 401a, 402t, 402g, 402c, 402a, 403c, 403g, 403a, 404t, 404g, |
| | | | 404a, 404c, 405g, 405c, 405a, 405t, 406t, 406g, 406c, 406a, 407t, 407g, 407c, 407a, 408c, 408g, 408a, 408t, 409g, 409c, 409t, 409a, 410c, |
| | | | 410t, 410g, 410a, 411t, 411g, 411a, 411c, 412g, 412c, 412a, 412t, 413g, 413c, 413a, 414g, 414c, 414a, 414t, 415g, 415c, 415a, 415t, 416g, |
| | | | 416c, 416a, 417c, 417t, 417g, 417a, 418c, 418t, 418g, 418a, 419g, 419c, 419t, 419a, 420g, 420c, 420a, 420t, 421g, 421c, 421t, 421a, 422c, |
| | | | 422g, 422a, 422t, 423t, 423g, 423c, 423a, 424g, 424c, 424a, 425t, 425g, 425c, 425a, 426t, 426c, 426g, 426a, 427g, 427c, 427a, 427t, 428g, |
| | | | 428c, 428a, 428t, 429g, 429c, 429a, 429t, 430g, 430c, 430a, 431g, 431c, 431a, 431t, 432g, 432c, 432a, 433g, 433c, 433a, 434c, 434g, 434t, |
| | | | 434a, 435a, 435g, 435c, 435t, 436a, 436c, 436g, 436t, 437a, 437c, 437g, 438g, 438a, 439a, 439g, 439c, 439t, 440t, 440a, 440c, 440g, 441g, |
| | | | 441a, 441c, 441t, 442a, 442c, 442g, 442t, 443a, 443c, 443g, 443t, 444g, 444a, 444c, 444t, 445a, 445c, 445g, 445t, 446a, 446g, 446c, 446Δ, |
| | | | 447a, 447g, 447t, 447c, 448a, 448c, 448g, 448t, 449a, 449g, 449c, 449t, 450a, 450g, 450c, 450t, 451a, 451c, 451g, 451Δ, 452a, 452c, 452g, |
| | | | 452Δ, 452t, 453a, 453g, 453c, 453Δ, 453t, 454a, 454c, 454g, 454Δ, 454t, 455a, 455g, 455c, 455Δ, 455t, 456a, 456c, 456g, 456Δ, 456t, |
| | | | 457a, 457c, 457g, 457Δ, 457t, 458a, 458c, 458g, 459a, 459c, 459g, 460a, 460c, 460g, 460Δ, 460t, 461a, 461g, 461c, 461Δ, 462a, 462c, |
| | | | 462g, 462Δ, 463a, 463g, 463c, 463Δ, 464a, 464c, 464g, 464Δ, 465c, 465g, 465Δ, 465a, 466t, 466a, 466c, 466g, 466Δ, 467a, 467c, 467g, |
| | | | 467Δ, 468a, 468g, 468c, 468Δ, 468t, 469a, 469g, 469c, 469t, 470a, 470g, 470c, 470Δ, 471c, 471a, 471g, 471t, 472c, 472g, 472a, 472t, 473a, |
| | | | 473c, 473g, 473t, 474a, 474c, 474g, 474t, 475a, 475c, 475g, 475t, 476a, 476c, 476g, 477a, 477c, 477g, 478a, 478c, 478g, 478t, 479a, 479c, |
| | | | 479g, 480a, 480c, 480g, 480t, 481c, 481a, 481g, 481t, 482a, 482c, 482g, 482t, 483a, 483c, 483g, 483Δ, 484a, 484t, 484c, 484g, 485a, 485t, |
| | | | 485c, 485g, 486a, 486c, 486g, 486Δ, 486t, 487a, 487c, 487g, 487Δ, 487t, 488a, 488t, 488c, 488g, 489c, 489a, 489g, 489t, 490a, 490t, 490c, |
| | | | 490g, 491a, 491c, 491g, 491t, 492a, 492c, 492g, 492Δ, 492t, 493a, 493c, 493g, 493t, 494c, 494a, 494g, 494t, 495a, 495c, 495g, 495t, 496c, |
| | | | 496g, 496a, 496t, 497c, 497g, 497a, 497Δ, 497t, 498c, 498g, 498a, 498Δ, 498t, 499c, 499g, 499a, 499t, 500c, 500g, 500t, 501c, 501g, 501a, |
| | | | 501Δ, 501t, 502c, 502g, 502a, 502Δ, 502t, 503c, 503g, 503a, 503Δ, 503t, 504g, 504c, 504a, 504t, 504Δ, 505g, 505c, 505a, 505t, 506c, 506g, |
| | | | 506a, 506t, 507c, 507g, 507a, 508t, 508c, 508g, 508a, 508Δ, 509g, 509c, 509a, 509Δ, 509t, 510c, 510g, 510a, 510Δ, 510t, 511c, 511g, 511a, |
| | | | 511t, 512t, 512g, 512c, 512a, 512Δ, 513g, 513c, 513a, 513t, 513Δ, 514c, 514a, 514Δ, 514g, 515c, 515a, 515Δ, 515t, 515g, 516a, 516Δ, |
| | | | 516t, 516c, 516g, 517a, 517Δ, 517g, 518a, 518t, 518Δ, 518c, 518g, 519t, 519a, 519c, 519g, 520c, 520a, 520t, 520g, 521a, 521t, 521g, 522a, |
| | | | 522Δ, 522t, 522c, 522g, 523a, 523Δ, 523g, 523t, 523c, 524g, 524c, 524a, 525a, 525Δ, 525t, 525c, 525g, 526t, 526g, 526c, 526a, 527t, 527a, |
| | | | 527c, 527g, 528t, 528c, 528g, 528a, 529Δ, 529t, 529c, 529a, 529g, 530Δ, 530t, 530g, 530c, 530a, 531t, 531g, 531c, 531a, 532Δ, 532t, 532g, |
| | | | 532c, 532a, 533Δ, 533t, 533g, 533c, 533a, 534Δ, 534t, 534c, 534g, 534a, 535Δ, 535t, 535g, 535a, 535c, 536t, 536Δ, 536g, 536c, 536a, |
| | | | 537Δ, 537t, 537g, 537c, 537a, 538a, 538Δ, 538t, 538c, 538g, 539Δ, 539t, 539c, 539g, 539a, 540Δ, 540t, 540a, 540c, 540g, 541g, 541Δ, |
| | | | 541t, 541a, 541c, 542Δ, 542t, 542g, 542c, 542a, 543t, 543Δ, 543g, 543c, 543a, 544a, 544t, 544g, 544Δ, 544c, 545a, 545Δ, 545t, 545g, 545c, |
| | | | 546t, 546a, 546Δ, 546g, 546c, 547Δ, 547t, 547c, 547g, 547a, 548t, 548a, 548Δ, 548g, 548c, 549Δ, 549g, 549c, 549a, 550a, 550Δ, 550g, |
| | | | 550c, 551Δ, 551g, 551a, 551c, 552t, 552Δ, 552g, 552a, 552c, 553a, 553Δ, 553g, 553c, 554a, 554Δ, 554c, 554g, 555Δ, 555a, 555g, 555c, |
| | | | 556a, 556Δ, 556g, 556c, 557Δ, 557g, 557t, 557c, 557a, 558a, 558Δ, 558g, 558c, 559t, 559Δ, 559a, 559c, 559g, 560t, 560Δ, 560c, 560g, |
| | | | 560a, 561a, 561Δ, 561c, 561g, 562a, 562c, 563g, 563c, 563a, 564g, 564c, 564a, 564t, 565g, 565c, 565a, 566g, 566c, 566a, 567a, 567g, |
| | | | 567c, 568t, 568g, 568a, 568c, 569a, 569g, 569c, 570a, 570g, 570c, 571g, 571c, 571a, 572c, 572a, 572t, 573g, 573c, 573a, 574g, 574c, 574a, |
| | | | 575g, 575c, 575a, 576g, 576c, 576a, 576t, 577g, 577c, 577a, 578a, 578g, 578c, 579g, 579c, 579a, 579t, 580g, 580c, 580a, 580t, 581g, 581c, |
| | | | 581a, 582g, 582c, 582a, 583g, 583c, 583a, 583t, 584g, 584c, 584a, 585g, 585a, 585c, 586g, 586a, 586c, 587g, 587a, 587c, 588g, 588a, |
| | | | 588c, 589g, 589a, 589c, 590g, 590c, 590a, 591c, 591g, 591a, 592g, 592a, 592c, 593g, 593c, 593a, 594g, 594a, 594c, 595g, 595a, 595c, |
| | | | 595t, 596g, 596a, 596c, 597c, 597a, 597g, 598g, 598a, 598c, 599g, 599a, 599c, 599t, 600g, 600c, 600a, 600t, 601c, 601g, 601a, 601t, 602g, |
| | | | 602a, 602c, 603a, 603c, 603g, 604g, 604a, 604c, 605a, 605c, 605g, 605t, 606a, 606c, 606g, 607c, 607a, 607g, 608g, 608c, 608a, 609a, |
| | | | 609c, 609g, 610c, 610a, 610g, 611t, 611c, 611g, 611a, 612c, 612a, 613a, 613c, 613g, 613t, 614a, 614c, 614g, 615g, 615a, 615c, 616g, 616t, |
| | | | 616a, 616c, 617t, 617a, 617c, 617g, 618c, 618a, 618g, 618t, 619a, 619c, 619g, 619t, 620g, 620c, 620a, 620t, 621g, 621a, 621c, 622a, 622c, |
| | | | 622g, 623a, 623c, 623g, 624g, 624c, 624a, 625a, 625c, 625g, 625t, 626a, 626c, 626g, 626t, 627a, 627c, 627g, 627t, 628c, 628a, 628g, 629c, |
| | | | 629a, 629g, 630a, 630g, 630c, 630t, 631a, 631c, 631g, 632a, 632c, 632g, 632t, 633a, 633c, 633g, 634a, 634c, 634g, 635c, 635a, 635g, 635t, |
| | | | 636c, 636a, 636g, 636t, 637a, 637c, 637g, 638a, 638c, 638g, 638t, 639a, 639c, 639g, 639t, 640a, 640c, 640g, 640t, 641c, 641a, 641g, 641t, |
| | | | 642c, 642g, 642a, 643c, 643a, 643g, 643t, 644c, 644g, 644a, 644t, 645g, 645c, 645a, 645t, 646a, 646g, 646c, 646t, 647g, 647a, 647t, 647c, |
| | | | 648a, 648g, 648c, 648t, 649a, 649g, 649c, 649t, 650a, 650g, 651g, 651a, 652g, 652a, 653a, 653g, 654a, 654c, 654g, 655g, 655a, 655c, |
| | | | 656g, 656a, 657a, 657g, 657t, 658a, 658t, 659g, 659a, 659t, 660a, 660g, 660t, 661g, 661a, 661c, 662a, 662c, 662g, 662t, 663a, 663g, 663t, |
| | | | 664a, 664c, 664g, 664t, 665a, 665c, 665t, 666g, 666a, 666c, 666t, 667a, 667c, 667g, 668a, 668c, 669a, 669c, 669g, 669t, 670a, 670g, 671a, |
| | | | 671c, 671g, 671t, 672g, 672a, 672c, 672t, 673g, 673a, 673c, 673t, 674g, 674a, 674c, 675a, 675c, 676g, 676c, 676a, 676t, 677c, 677g, 677a, |
| | | | 677t, 678g, 678c, 678a, 678t, 679g, 679c, 679a, 679t, 680c, 680g, 680a, 680t, 681c, 681g, 681a, 681t, 682g, 682c, 682a, 682t, 683g, 683c, |
| | | | 683t, 683a, 684c, 684g, 684a, 684t, 685c, 685g, 685a, 685t, 686c, 686g, 686a, 686t, 687c, 687g, 687a, 687t, 688c, 688g, 688a, 688t, 689g, |
| | | | 689c, 689a, 689t, 690g, 690c, 690a, 690t, 691c, 691g, 691a, 691t, 692c, 692g, 692a, 692t, 693c, 693g, 693a, 693t, 694c, 694g, 694a, 694t, |
| | | | 695g, 695a, 695t, 696g, 696a, 696t, 697g, 697a, 697t, 698g, 698a, 699a, 699t, 700a, 700t, 701a, 701t, 702a, 702t, 703t, 703a, 704a, 704t, |
| | | | 705a, 706a, 706g, 706t, 707a, 707t, 708a, 709a, 709t, 710a, 710t, 711c, 711a, 711t, 712a, 712t, 713a, 713t, 714a, 714t, 715g, 715a, 716a, |
| | | | 716t, 717a, 717t, 718a, 718t, 719a, 719t, 720a, 720t, 721a, 721t, 722a, 722t, 723a, 723g, 723t, 724t, 725t, 726t, 727t, 728t, 729t, 730t, 731a, |
| | | | 731t, 732t, 733t, 734t, 735t, 736t, 737t, 738t, 67_68ins_g, 67_68ins_n, 67_68ins_t, 67_68ins_a, 78_79ins_t, 78_79ins_a, 127_128ins_g, |
| | | | 127_128ins_a, 127_128ins_t, 177_178ins_t, 177_178ins_a, 227_228ins_t, 227_228ins_a, 230_231ins_g, 230_231ins_a, 230_231ins_t, |
| | | | 231_232ins_g, 231_232ins_a, 231_232ins_t, 232_233ins_g, 232_233ins_a, 232_233ins_t, 233_234ins_g, 233_234ins_a, 233_234ins_t, |
| | | | 234_235ins_g, 234_235ins_a, 234_235ins_t, 235_236ins_g, 235_236ins_a, 235_236ins_t, 236_237ins_g, 236_237ins_a, 236_237ins_t, |
| | | | 237_238ins_g, 237_238ins_a, 237_238ins_t, 238_239ins_t, 238_239ins_a, 242_243ins_t, 242_243ins_a, 244_245ins_t, 244_245ins_a, |
| | | | 246_247ins_a, 246_247ins_t, 252_253ins_g, 252_253ins_a, 252_253ins_t, 256_257ins_g, 256_257ins_a, 256_257ins_t, 261_262ins_g, |
| | | | 261_262ins_a, 261_262ins_t, 267_268ins_g, 267_268ins_a, 267_268ins_t, 268_269ins_t, 268_269ins_a, 270_271ins_g, 270_271ins_a, |
| | | | 270_271ins_t, 271_272ins_g, 271_272ins_a, 271_272ins_t, 272_273ins_g, 272_273ins_a, 272_273ins_t, 275_276ins_t, 275_276ins_a, |
| | | | 277_278ins_g, 277_278ins_a, 277_278ins_t, 278_279ins_g, 278_279ins_a, 278_279ins_t, 279_280ins_g, 279_280ins_a, 279_280ins_t, |
| | | | 281_282ins_g, 281_282ins_a, 281_282ins_t, 281_282ins_n, 283_284ins_g, 283_284ins_a, 283_284ins_t, 283_284ins_n, 284_285ins_g, |
| | | | 284_285ins_a, 284_285ins_t, 285_286ins_g, 285_286ins_a, 285_286ins_t, 286_287ins_g, 286_287ins_a, 286_287ins_t, 291_292ins_g, |
| | | | 291_292ins_a, 291_292ins_t, 292_293ins_g, 292_293ins_a, 292_293ins_t, 295_296ins_t, 295_296ins_a, 298_299ins_g, 298_299ins_a, |
| | | | 298_299ins_t, 299_300ins_g, 299_300ins_a, 299_300ins_t, 301_302ins_g, 301_302ins_a, 301_302ins_t, 302_303ins_g, 302_303ins_a, |
| | | | 302_303ins_t, 304_305ins_g, 304_305ins_a, 304_305ins_t, 305_306ins_g, 305_306ins_a, 305_306ins_t, 306_307ins_t, 306_307ins_n, |
| | | | 307_308ins_t, 307_308ins_g, 309_310ins_n, 309_310ins_a, 311_312ins_t, 314_315ins_a, 314_315ins_t, 315_316ins_g, 315_316ins_a, |
| | | | 315_316ins_t, 316_317ins_t, 316_317ins_a, 318_319ins_t, 318_319ins_a, 320_321ins_t, 320_321ins_a, 321_322ins_a, 321_322ins_t, |
| | | | 322_323ins_a, 322_323ins_t, 323_324ins_t, 323_324ins_a, 324_325ins_a, 324_325ins_t, 325_326ins_t, 325_326ins_a, 326_327ins_a, |
| | | | 326_327ins_t, 327_328ins_t, 327_328ins_a, 327_328ins_c, 328_329ins_t, 328_329ins_a, 328_329ins_c, 329_330ins_t, 329_330ins_a, |
| | | | 329_330ins_c, 330_331ins_a, 330_331ins_t, 330_331ins_c, 331_332ins_t, 331_332ins_a, 332_333ins_t, 332_333ins_a, 333_334ins_t, |
| | | | 333_334ins_a, 334_335ins_t, 334_335ins_a, 335_336ins_t, 335_336ins_a, 336_337ins_t, 336_337ins_a, 337_338ins_a, 337_338ins_t, |
| | | | 338_339ins_a, 338_339ins_t, 339_340ins_t, 339_340ins_a, 340_341ins_t, 340_341ins_a, 341_342ins_t, 341_342ins_a, 342_343ins_t, |
| | | | 342_343ins_a |
| 231 | antis_3p_ZEBOV | 315 | 4a, 6g, 6Δ, 7g, 7t, 7a, 8g, 8c, 9t, 10g, 10a, 12a, 12g, 13g, 13t, 14a, 14g, 15t, 15c, 17c, 17g, 17t, 18c, 18a, 19t, 19a, 20t, 20g, 20a, 22c, 22t, |
| | | | 23t, 23Δ, 24g, 24Δ, 25t, 25c, 25a, 26c, 26Δ, 27g, 29t, 29a, 30c, 30a, 32t, 32c, 33c, 33a, 34a, 35c, 36c, 36a, 38g, 38t, 39t, 41g, 41a, 41c, 44c, |
| | | | 44a, 45g, 45c, 46t, 46c, 47g, 47a, 48g, 48t, 48a, 49a, 50c, 50g, 50a, 51c, 51t, 52c, 52t, 52g, 53c, 53g, 53a, 54g, 54a, 55c, 55g, 55a, 56t, 56g, |
| | | | 56Δ, 57t, 57a, 57g, 60t, 61g, 61a, 62t, 62c, 63t, 63c, 63Δ, 64g, 66c, 67g, 67a, 68a, 69a, 69t, 70a, 70c, 71a, 71g, 72c, 73t, 73a, 74a, 74g, 75c, |
| | | | 77g, 77c, 77a, 78g, 79t, 79c, 80c, 81c, 82c, 82g, 82t, 84g, 84c, 85g, 85c, 86a, 86c, 86t, 86g, 86Δ, 87t, 89t, 89c, 90t, 92g, 93g, 94g, 95a, 96a, |
| | | | 97g, 98a, 99g, 100g, 101g, 108g, 110t, 111g, 113g, 113a, 114a, 114c, 115a, 118g, 123c, 128a, 129a, 129g, 129c, 131a, 142g, 142a, 143c, |
| | | | 145a, 146g, 146c, 147c, 148t, 150g, 153c, 154t, 155a, 156t, 156c, 157g, 158t, 158a, 160g, 161c, 162c, 162t, 163a, 163g, 164g, 166a, 167g, |
| | | | 168c, 168g, 169g, 170g, 170t, 172t, 172a, 174g, 174c, 175g, 176a, 176c, 181g, 182g, 182c, 183a, 187a, 189c, 190a, 192c, 193c, 196t, 196c, |
| | | | 202a, 205a, 205t, 206g, 206c, 207t, 208g, 209a, 210a, 212a, 213c, 213a, 214a, 215a, 216g, 218t, 219c, 222g, 222c, 223a, 224g, 224a, 227c, |
| | | | 227g, 229c, 233a, 234g, 240a, 240c, 254c, 255c, 256c, 257c, 258t, 264g, 269g, 273g, 274c, 280a, 280c, 282t, 283c, 285t, 288c, 289t, 290a, |
| | | | 292g, 295t, 298g, 300a, 301c, 308t, 315g, 317c, 320c, 321a, 323a, 327t, 332t, 337a, 343a, 344c, 354g, 356a, 362t, 363g, 365c, 374t, 381g, |
| | | | 382g, 387g, 388g, 389a, 397g, 398g, 400c, 403a, 408t, 420a, 422c, 423c, 425a, 428t, 428a, 429g, 429t, 437g, 437a, 440Δ, 441g, 445t, 448t, |
| | | | 451t, 451g, 466c, 466a, 467g, 48_49ins_t, 50_51ins_a |
| 232 | antis_5p_SEBOV | 225 | 56c, 61t, 61g, 62c, 64c, 64t, 65c, 70c, 70a, 71c, 71t, 71g, 74t, 77t, 80t, 80a, 85t, 86t, 87g, 88t, 89t, 95g, 96g, 96a, 97t, 98t, 100t, 104a, 105a, |
| | | | 105t, 108t, 109t, 113t, 113g, 114t, 139g, 143a, 147g, 148g, 151a, 152a, 186g, 190a, 194g, 195g, 195c, 198a, 199a, 202g, 210g, 211g, 213t, |
| | | | 215a, 215c, 221t, 221c, 222t, 223a, 224a, 229c, 230c, 238t, 241t, 241g, 246t, 247t, 255t, 256t, 267g, 270g, 275g, 276g, 278g, 279g, 283t, |
| | | | 289t, 297t, 298t, 307g, 309t, 315g, 316g, 317t, 318t, 318c, 326t, 327t, 327g, 334c, 342c, 342g, 343c, 348g, 353g, 356g, 357g, 361g, 362g, |
| | | | 395t, 397a, 403t, 404t, 405a, 405t, 406a, 410t, 411t, 415t, 421t, 421g, 429t, 430t, 436a, 436g, 446a, 446g, 447g, 448c, 448a, 449c, 452a, |
| | | | 454c, 455g, 456g, 457c, 458c, 458a, 460a, 461a, 462c, 462g, 463c, 468a, 470g, 470a, 471g, 471a, 471c, 474a, 477a, 480a, 480c, 482a, |
| | | | 483a, 484a, 490a, 490c, 492a, 493a, 496a, 497c, 504a, 505c, 505a, 505g, 506c, 506g, 513g, 513c, 514c, 514g, 515c, 532g, 537g, 539t, |
| | | | 539c, 539a, 540g, 540t, 541g, 545g, 545a, 545t, 547c, 548c, 553a, 554a, 554g, 560g, 568g, 569g, 571t, 573c, 576g, 579t, 580t, 584g, 585g, |
| | | | 585c, 598g, 601c, 603t, 606g, 607g, 609c, 610c, 611t, 612t, 621a, 628t, 628c, 636t, 637t, 654c, 660t, 662c, 662g, 663c, 670t, 673t, 676t, |
| | | | 681t, 681g, 682t, 682g, 685t, 686t, 688g, 689g, 690g, 693t, 694t, 695t, 696g, 697g |
| 236 | antis_3p_SEBOV | 58 | 4Δ, 6g, 10t, 11c, 15g, 16a, 26t, 31t, 44g, 45g, 46a, 48c, 48a, 49Δ, 50g, 54t, 55t, 56c, 57c, 57Δ, 60t, 60g, 61c, 72a, 73a, 73c, 75c, 75a, 76g, |
| | | | 90g, 91g, 92c, 106a, 112t, 120t, 127a, 128g, 135a, 144t, 148t, 151g, 166t, 176g, 179a, 194a, 200t, 214c, 253a, 256a, 277t, 312g, 362t, 424g, |
| | | | 426a, 428a, 7_8ins_a, 41_42ins_t, 44_45ins_g |
| 237 | antis_5p_MARV | 517 | 9t, 9c, 10t, 10g, 21a, 21t, 30t, 30g, 30a, 32g, 39g, 43t, 48t, 48c, 53c, 55c, 55g, 71t, 71c, 72a, 72g, 73t, 73c, 75a, 76t, 76c, 88g, 88a, 89c, 89g, |
| | | | 89a, 91t, 91a, 93t, 93a, 95t, 100t, 101a, 109g, 109a, 109Δ, 109c, 111g, 111c, 113c, 115g, 118g, 118a, 119t, 119a, 123t, 123a, 124g, 126a, |
| | | | 131g, 131a, 133g, 133c, 135t, 138a, 141g, 141c, 143t, 144t, 144g, 145t, 145g, 145a, 146g, 146a, 148a, 148t, 150a, 150g, 151a, 151t, 152t, |
| | | | 152a, 153g, 153t, 153a, 155a, 155g, 155c, 160a, 160c, 162g, 163c, 163t, 164c, 164a, 166c, 168c, 168Δ, 168t, 169a, 169t, 170t, 170a, 171a, |
| | | | 171c, 172t, 172a, 176c, 177t, 177c, 178c, 178a, 178g, 179a, 179c, 179g, 180g, 183t, 184c, 184a, 186c, 186g, 187g, 188c, 188Δ, 189Δ, 189c, |
| | | | 190Δ, 194c, 194t, 197c, 197Δ, 203g, 205g, 205a, 205c, 206a, 206g, 207a, 207g, 208g, 208a, 211t, 211a, 213a, 213Δ, 214a, 215g, 220a, |
| | | | 220t, 221t, 221g, 224c, 226a, 226c, 227g, 228t, 228g, 229g, 229c, 230g, 233a, 236g, 237t, 237g, 237a, 239c, 242c, 244g, 248c, 249a, 250t, |
| | | | 251g, 251c, 252a, 252c, 253c, 254c, 255g, 255c, 257a, 257t, 258g, 258t, 261c, 261g, 261t, 262c, 262g, 263a, 263c, 264c, 264t, 266t, 266g, |
| | | | 267g, 267t, 268g, 268c, 271c, 275g, 275c, 278a, 278t, 278c, 279a, 279g, 279c, 282c, 283g, 283a, 285c, 285a, 286a, 286c, 286t, 287t, 287g, |
| | | | 292t, 293c, 297a, 298c, 298t, 300t, 300c, 302c, 303c, 303t, 303g, 305g, 305c, 306c, 306t, 307c, 307t, 308t, 309t, 309g, 310c, 310g, 312g, |
| | | | 314c, 314g, 315t, 315c, 316t, 321a, 321g, 323a, 330c, 330t, 332t, 339g, 339c, 340c, 344t, 344c, 347t, 347c, 350g, 350c, 351g, 351c, 352a, |
| | | | 352c, 352g, 353t, 353g, 354t, 354g, 356c, 356a, 356t, 357a, 358g, 359c, 360c, 360a, 362g, 362t, 363c, 364a, 364c, 366c, 366t, 366g, 368t, |
| | | | 370c, 373t, 373c, 374t, 374g, 375t, 375g, 375a, 376g, 376t, 377t, 377g, 378t, 380a, 380c, 382t, 382a, 383g, 383a, 384c, 384t, 385t, 385c, |
| | | | 386c, 387c, 387t, 387a, 387g, 389a, 389c, 391a, 391g, 392g, 393c, 393g, 394c, 394t, 395g, 398a, 399c, 399g, 400g, 400c, 400t, 401g, 401c, |
| | | | 404a, 404t, 405a, 405t, 406t, 407t, 408c, 408t, 408a, 409t, 412a, 412c, 413t, 413a, 414c, 414t, 415t, 415g, 416t, 416g, 417c, 417g, 420a, |
| | | | 420g, 420t, 421g, 421t, 422t, 423t, 423g, 424g, 424c, 428g, 428a, 428c, 429t, 429a, 430a, 430c, 431Δ, 431g, 434a, 434t, 436a, 436t, 437a, |
| | | | 437c, 441c, 441t, 445t, 450t, 450c, 451a, 451t, 452t, 452c, 453a, 454c, 454a, 455g, 455a, 456a, 456t, 457c, 457g, 459c, 460c, 461a, 461t, |
| | | | 462c, 463t, 463a, 463c, 465a, 466c, 468a, 472a, 472t, 472g, 473c, 473g, 473t, 474g, 474t, 476t, 477t, 478t, 478g, 479g, 481g, 482a, 483t, |
| | | | 485t, 488c, 491a, 491c, 493c, 493g, 494g, 494c, 495g, 495t, 500c, 500g, 502g, 504c, 507g, 508c, 511a, 512t, 515a, 515c, 515t, 516c, 516t, |
| | | | 517t, 517g, 517Δ, 522t, 523c, 523t, 524t, 527c, 530t, 530g, 531g, 532c, 538g, 538c, 542g, 542t, 543t, 543c, 543a, 544t, 549g, 551t, 554c, |
| | | | 556c, 558c, 559c, 559t, 560c, 563c, 563a, 565a, 565c, 566t, 567Δ, 567c, 567t, 571t, 571a, 572a, 572g, 576c, 576t, 578c, 579g, 579t, 581t, |
| | | | 583c, 585c, 586t, 587c, 588t, 588Δ, 588g, 591g, 591a, 600a, 604c, 605c, 608a, 625Δ, 625t, 627t, 632Δ, 186_187ins_c, 193_194ins_c, |
| | | | 232_233ins_c, 234_235ins_t, 239_240ins_c, 423_424ins_c, 428_429ins_t, 430_431ins_t, 430_431ins_c, 564_565ins_c |
| 240 | antis_3p_MARV | 12 | 14a, 16Δ, 16a, 21t, 27t, 33t, 58c, 76g, 78a, 81a, 82g, 18_19ins_c |
| 242 | antis_3p_RV | 3894 | 3g, 4c, 4a, 4Δ, 4g, 4t, 5c, 5g, 5a, 5Δ, 5t, 6c, 6g, 6Δ, 6a, 6t, 7t, 7g, 7Δ, 7c, 7a, 8t, 8Δ, 8c, 8a, 8g, 9Δ, 9g, 9c, 9a, 9t, 10t, 10g, 10Δ, 10c, 10a, |
| | | | 11Δ, 11c, 11a, 11g, 11t, 12t, 12g, 12Δ, 12c, 12a, 13a, 13c, 13Δ, 13t, 13g, 14Δ, 14g, 14c, 14t, 14a, 15g, 15Δ, 15t, 15c, 15a, 16g, 16a, 16c, |
| | | | 16t, 16Δ, 17t, 17Δ, 17c, 17g, 17a, 18Δ, 18a, 18t, 18g, 18c, 19Δ, 19a, 19c, 19t, 19g, 20a, 20c, 20Δ, 20g, 20t, 21t, 21Δ, 21c, 21a, 21g, 22c, |
| | | | 22g, 22a, 22Δ, 22t, 23c, 23a, 23t, 23g, 23Δ, 24a, 24t, 24Δ, 24c, 24g, 25t, 25Δ, 25a, 25g, 25c, 26t, 26a, 26g, 26c, 26Δ, 27a, 27Δ, 27g, 27t, |
| | | | 27c, 28t, 28a, 28g, 28c, 28Δ, 29t, 29Δ, 29a, 29c, 29g, 30a, 30g, 30Δ, 30t, 30c, 31g, 31a, 31t, 31Δ, 31c, 32Δ, 32g, 32t, 32c, 32a, 33Δ, 33a, |
| | | | 33g, 33t, 33c, 34a, 34Δ, 34t, 34g, 34c, 35a, 35t, 35g, 35Δ, 35c, 36a, 36t, 36Δ, 36g, 36c, 37t, 37Δ, 37g, 37a, 37c, 38a, 38t, 38Δ, 38c, 38g, 39t, |
| | | | 39a, 39g, 39Δ, 39c, 40a, 40Δ, 40g, 40t, 40c, 41a, 41Δ, 41t, 41g, 41c, 42t, 42a, 42Δ, 42c, 42g, 43Δ, 43a, 43t, 43g, 43c, 44Δ, 44t, 44a, 44g, |
| | | | 44c, 45Δ, 45a, 45t, 45g, 45c, 46a, 46Δ, 46g, 46t, 46c, 47Δ, 47g, 47a, 47t, 47c, 48a, 48Δ, 48c, 48g, 48t, 49t, 49a, 49Δ, 49c, 49g, 50c, 50Δ, |
| | | | 50g, 50a, 50t, 51Δ, 51a, 51g, 51t, 51c, 52a, 52t, 52c, 52Δ, 52g, 53g, 53Δ, 53a, 53t, 53c, 54a, 54c, 54g, 54t, 54Δ, 55g, 55c, 55t, 55a, 55Δ, |
| | | | 56Δ, 56g, 56a, 56c, 56t, 57a, 57t, 57c, 57Δ, 57g, 58Δ, 58a, 58c, 58g, 58t, 59g, 59c, 59t, 59Δ, 59a, 60t, 60a, 60g, 60c, 60Δ, 61g, 61a, 61Δ, |
| | | | 61t, 61c, 62g, 62c, 62Δ, 62t, 62a, 63g, 63t, 63c, 63a, 63Δ, 64t, 64g, 64c, 64a, 64Δ, 65t, 65a, 65Δ, 65c, 65g, 66g, 66a, 66t, 66c, 66Δ, 67g, |
| | | | 67Δ, 67t, 67c, 67a, 68t, 68c, 68g, 68Δ, 68a, 69g, 69t, 69a, 69c, 69Δ, 70g, 70c, 70a, 70t, 70Δ, 71a, 71c, 71t, 71g, 71Δ, 72t, 72a, 72g, 72Δ, |
| | | | 72c, 73t, 73c, 73g, 73a, 73Δ, 74c, 74t, 74a, 74Δ, 74g, 75t, 75g, 75a, 75c, 75Δ, 76c, 76g, 76t, 76a, 76Δ, 77c, 77t, 77g, 77a, 77Δ, 78c, 78g, |
| | | | 78t, 78a, 78Δ, 79a, 79g, 79t, 79c, 79Δ, 80c, 80g, 80t, 80a, 80Δ, 81c, 81a, 81g, 81t, 81Δ, 82a, 82c, 82g, 82t, 82Δ, 83g, 83c, 83t, 83a, 83Δ, |
| | | | 84g, 84a, 84t, 84Δ, 84c, 85g, 85a, 85t, 85c, 85Δ, 86g, 86t, 86a, 86c, 86Δ, 87g, 87t, 87a, 87c, 87Δ, 88g, 88a, 88c, 88Δ, 88t, 89a, 89t, 89c, |
| | | | 89g, 89Δ, 90t, 90a, 90g, 90c, 90Δ, 91c, 91a, 91g, 91t, 91Δ, 92c, 92a, 92t, 92g, 92Δ, 93a, 93c, 93t, 93g, 93Δ, 94t, 94a, 94g, 94Δ, 94c, 95g, |
| | | | 95a, 95c, 95t, 95Δ, 96c, 96g, 96t, 96a, 96Δ, 97a, 97c, 97t, 97g, 97Δ, 98g, 98t, 98a, 98c, 98Δ, 99a, 99t, 99c, 99g, 99Δ, 100g, 100c, 100a, |
| | | | 100t, 100Δ, 101c, 101g, 101t, 101a, 101Δ, 102g, 102a, 102c, 102t, 102Δ, 103g, 103t, 103c, 103Δ, 103a, 104g, 104c, 104a, 104t, 104Δ, |
| | | | 105g, 105c, 105a, 105t, 105Δ, 106c, 106g, 106t, 106a, 106Δ, 107c, 107t, 107g, 107a, 107Δ, 108a, 108t, 108c, 108Δ, 108g, 109g, 109c, |
| | | | 109Δ, 109t, 109a, 110a, 110Δ, 110c, 110t, 110g, 111t, 111c, 111g, 111Δ, 111a, 112t, 112g, 112c, 112a, 112Δ, 113g, 113c, 113a, 113Δ, |
| | | | 113t, 114c, 114t, 114a, 114Δ, 114g, 115a, 115c, 115g, 115Δ, 115t, 116t, 116g, 116Δ, 116c, 116a, 117t, 117Δ, 117c, 117a, 117g, 118c, 118g, |
| | | | 118t, 118Δ, 118a, 119c, 119g, 119t, 119a, 119Δ, 120g, 120t, 120c, 120Δ, 120a, 121t, 121a, 121Δ, 121c, 121g, 122t, 122Δ, 122c, 122g, |
| | | | 122a, 123a, 123t, 123g, 123Δ, 123c, 124t, 124Δ, 124g, 124c, 124a, 125g, 125t, 125c, 125a, 125Δ, 126c, 126a, 126t, 126Δ, 126g, 127a, |
| | | | 127Δ, 127t, 127c, 127g, 128a, 128t, 128c, 128Δ, 128g, 129t, 129c, 129Δ, 129g, 129a, 130g, 130Δ, 130t, 130a, 130c, 131c, 131a, 131g, |
| | | | 131Δ, 131t, 132g, 132a, 132t, 132Δ, 132c, 133t, 133g, 133c, 133a, 133Δ, 134c, 134t, 134g, 134Δ, 134a, 135c, 135t, 135a, 135g, 135Δ, |
| | | | 136c, 136g, 136t, 136a, 136Δ, 137g, 137t, 137c, 137a, 137Δ, 138g, 138t, 138a, 138c, 138Δ, 139t, 139a, 139g, 139c, 139Δ, 140a, 140c, 140t, |
| | | | 140g, 140Δ, 141a, 141t, 141g, 141c, 141Δ, 142g, 142t, 142a, 142c, 142Δ, 143g, 143a, 143t, 143c, 143Δ, 144g, 144t, 144a, 144c, 144Δ, |
| | | | 145g, 145t, 145a, 145c, 145Δ, 146t, 146g, 146a, 146c, 146Δ, 147g, 147a, 147c, 147t, 147Δ, 148t, 148g, 148c, 148a, 148Δ, 149g, 149c, 149t, |
| | | | 149a, 149Δ, 150g, 150a, 150t, 150c, 150Δ, 151g, 151t, 151a, 151c, 151Δ, 152c, 152g, 152t, 152a, 152Δ, 153t, 153g, 153a, 153Δ, 153c, |
| | | | 154g, 154a, 154t, 154c, 154Δ, 155g, 155a, 155t, 155c, 155Δ, 156g, 156a, 156t, 156c, 156Δ, 157a, 157g, 157c, 157t, 157Δ, 158g, 158c, 158t, |
| | | | 158a, 158Δ, 159g, 159a, 159c, 159t, 159Δ, 160t, 160g, 160a, 160c, 160Δ, 161g, 161a, 161t, 161c, 161Δ, 162a, 162c, 162t, 162g, 162Δ, |
| | | | 163a, 163t, 163g, 163c, 163Δ, 164g, 164a, 164t, 164c, 164Δ, 165g, 165a, 165t, 165c, 165Δ, 166a, 166c, 166g, 166t, 166Δ, 167c, 167g, 167t, |
| | | | 167a, 167Δ, 168a, 168g, 168t, 168c, 168Δ, 169t, 169a, 169g, 169c, 169Δ, 170a, 170t, 170g, 170c, 170Δ, 171g, 171c, 171a, 171t, 171Δ, |
| | | | 172g, 172a, 172t, 172c, 172Δ, 173a, 173t, 173c, 173g, 173Δ, 174a, 174t, 174g, 174c, 174Δ, 175g, 175t, 175c, 175a, 175Δ, 176g, 176c, |
| | | | 176a, 176t, 176Δ, 177a, 177g, 177c, 177t, 177Δ, 178g, 178c, 178a, 178t, 178Δ, 179a, 179g, 179c, 179t, 179Δ, 180c, 180a, 180g, 180t, |
| | | | 180Δ, 181a, 181g, 181c, 181t, 181Δ, 182g, 182a, 182t, 182c, 182Δ, 183g, 183c, 183t, 183a, 183Δ, 184c, 184a, 184t, 184g, 184Δ, 185c, |
| | | | 185g, 185a, 185t, 185Δ, 186a, 186t, 186c, 186g, 186Δ, 187c, 187a, 187g, 187t, 187Δ, 188t, 188a, 188c, 188g, 188Δ, 189t, 189c, 189g, 189a, |
| | | | 189Δ, 190c, 190t, 190a, 190g, 190Δ, 191a, 191c, 191t, 191g, 191Δ, 192a, 192g, 192t, 192c, 192Δ, 193t, 193c, 193a, 193g, 193Δ, 194t, |
| | | | 194a, 194g, 194c, 195a, 195c, 195t, 195Δ, 195g, 196a, 196Δ, 196c, 196g, 196t, 197g, 197a, 197t, 197c, 197Δ, 198a, 198t, 198g, 198Δ, |
| | | | 198c, 199g, 199t, 199c, 199a, 199Δ, 200g, 200a, 200c, 200t, 200Δ, 201c, 201a, 201g, 201t, 201Δ, 202t, 202g, 202a, 202c, 202Δ, 203c, 203t, |
| | | | 203g, 203a, 203Δ, 204c, 204g, 204t, 204a, 204Δ, 205a, 205g, 205t, 205c, 205Δ, 206a, 206t, 206c, 206g, 206Δ, 207a, 207g, 207c, 207Δ, |
| | | | 207t, 208t, 208a, 208c, 208g, 208Δ, 209a, 209c, 209g, 209Δ, 209t, 210g, 210c, 210a, 210t, 210Δ, 211a, 211t, 211c, 211g, 211Δ, 212a, 212g, |
| | | | 212t, 212c, 212Δ, 213c, 213g, 213a, 213t, 213Δ, 214t, 214c, 214a, 214g, 214Δ, 215t, 215g, 215c, 215a, 215Δ, 216a, 216g, 216c, 216Δ, |
| | | | 216t, 217a, 217c, 217t, 217g, 217Δ, 218g, 218c, 218a, 218t, 218Δ, 219t, 219c, 219a, 219Δ, 219g, 220t, 220g, 220c, 220a, 220Δ, 221c, 221g, |
| | | | 221a, 221t, 221Δ, 222t, 222a, 222c, 222g, 222Δ, 223g, 223c, 223t, 223a, 223Δ, 224t, 224a, 224c, 224g, 224Δ, 225t, 225g, 225c, 225a, |
| | | | 225Δ, 226g, 226a, 226t, 226c, 226Δ, 227t, 227g, 227a, 227c, 227Δ, 228g, 228t, 228a, 228c, 228Δ, 229g, 229a, 229t, 229Δ, 229c, 230a, |
| | | | 230t, 230g, 230Δ, 230c, 231c, 231t, 231g, 231a, 231Δ, 232t, 232a, 232c, 232g, 232Δ, 233a, 233t, 233c, 233g, 233Δ, 234g, 234t, 234a, 234c, |
| | | | 234Δ, 235t, 235a, 235c, 235Δ, 235g, 236g, 236a, 236t, 236c, 236Δ, 237c, 237a, 237g, 237t, 237Δ, 238a, 238c, 238t, 238g, 238Δ, 239a, |
| | | | 239g, 239t, 239c, 239Δ, 240c, 240g, 240a, 240t, 240Δ, 241a, 241c, 241t, 241g, 241Δ, 242c, 242g, 242t, 242a, 242Δ, 243g, 243t, 243a, |
| | | | 243Δ, 243c, 244g, 244t, 244a, 244Δ, 244c, 245g, 245a, 245Δ, 245t, 245c, 246g, 246a, 246t, 246Δ, 246c, 247a, 247t, 247c, 247g, 247Δ, |
| | | | 248c, 248t, 248a, 248g, 248Δ, 249t, 249a, 249g, 249c, 249Δ, 250g, 250t, 250a, 250c, 250Δ, 251g, 251t, 251a, 251c, 251Δ, 252g, 252a, |
| | | | 252c, 252t, 252Δ, 253a, 253g, 253t, 253c, 253Δ, 254t, 254g, 254a, 254c, 254Δ, 255a, 255g, 255c, 255t, 255Δ, 256g, 256a, 256t, 256Δ, |
| | | | 256c, 257g, 257a, 257t, 257c, 257Δ, 258c, 258t, 258g, 258a, 258Δ, 259t, 259a, 259c, 259Δ, 259g, 260a, 260t, 260Δ, 260c, 260g, 261a, |
| | | | 261g, 261Δ, 261c, 261t, 262t, 262g, 262a, 262c, 262Δ, 263c, 263g, 263t, 263a, 263Δ, 264c, 264a, 264g, 264Δ, 264t, 265g, 265t, 265a, 265c, |
| | | | 265Δ, 266a, 266g, 266t, 266c, 266Δ, 267a, 267t, 267c, 267g, 267Δ, 268a, 268t, 268c, 268Δ, 268g, 269a, 269g, 269t, 269Δ, 269c, 270t, |
| | | | 270a, 270g, 270c, 270Δ, 271g, 271t, 271a, 271c, 271Δ, 272t, 272a, 272g, 272c, 272Δ, 273a, 273t, 273c, 273g, 273Δ, 274g, 274a, 274t, |
| | | | 274c, 274Δ, 275a, 275g, 275c, 275t, 275Δ, 276a, 276g, 276t, 276Δ, 276c, 277g, 277t, 277c, 277a, 277Δ, 278a, 278g, 278t, 278c, 278Δ, |
| | | | 279g, 279t, 279a, 279c, 279Δ, 280t, 280c, 280a, 280g, 280Δ, 281c, 281a, 281Δ, 281t, 281g, 282a, 282c, 282t, 282Δ, 282g, 283a, 283g, |
| | | | 283Δ, 283t, 283c, 284t, 284g, 284c, 284a, 284Δ, 285t, 285a, 285g, 285Δ, 285c, 286a, 286c, 286g, 286t, 286Δ, 287g, 287a, 287c, 287Δ, |
| | | | 287t, 288c, 288g, 288a, 288Δ, 288t, 289c, 289Δ, 289g, 289a, 289t, 290a, 290g, 290t, 290c, 290Δ, 291a, 291g, 291c, 291t, 291Δ, 292a, 292g, |
| | | | 292c, 292t, 292Δ, 293g, 293t, 293a, 293Δ, 293c, 294g, 294c, 294Δ, 294a, 294t, 295a, 295g, 295t, 295Δ, 295c, 296c, 296t, 296a, 296Δ, |
| | | | 296g, 297a, 297t, 297c, 297g, 297Δ, 298g, 298a, 298t, 298Δ, 298c, 299g, 299c, 299a, 299t, 299Δ, 300g, 300a, 300t, 300c, 300Δ, 301g, |
| | | | 301c, 301a, 301t, 301Δ, 302c, 302g, 302a, 302t, 302Δ, 303a, 303t, 303g, 303c, 303Δ, 304t, 304g, 304a, 304c, 304Δ, 305t, 305a, 305g, 305c, |
| | | | 305Δ, 306g, 306t, 306Δ, 306c, 306a, 307t, 307g, 307a, 307c, 307Δ, 308g, 308Δ, 308a, 308t, 308c, 309g, 309a, 309t, 309c, 309Δ, 310t, |
| | | | 310a, 310g, 310Δ, 310c, 311t, 311g, 311a, 311c, 311Δ, 312a, 312g, 312t, 312c, 312Δ, 313g, 313a, 313c, 313t, 313Δ, 314a, 314c, 314t, |
| | | | 314g, 314Δ, 315a, 315c, 315t, 315g, 315Δ, 316a, 316t, 316g, 316c, 316Δ, 317a, 317t, 317g, 317Δ, 317c, 318a, 318g, 318t, 318c, 318Δ, |
| | | | 319a, 319g, 319c, 319t, 319Δ, 320c, 320g, 320a, 320t, 320Δ, 321a, 321t, 321g, 321c, 321Δ, 322a, 322c, 322g, 322t, 322Δ, 323g, 323t, 323c, |
| | | | 323a, 323Δ, 324g, 324c, 324a, 324t, 324Δ, 325t, 325a, 325g, 325Δ, 325c, 326a, 326c, 326g, 326t, 326Δ, 327a, 327t, 327g, 327c, 327Δ, |
| | | | 328t, 328g, 328Δ, 328c, 328a, 329a, 329g, 329t, 329c, 329Δ, 330t, 330g, 330a, 330c, 330Δ, 331t, 331Δ, 331a, 331c, 331g, 332a, 332g, |
| | | | 332Δ, 332t, 332c, 333a, 333t, 333Δ, 333g, 333c, 334g, 334a, 334Δ, 334c, 334t, 335c, 335t, 335a, 335Δ, 335g, 336Δ, 336t, 336a, 336c, |
| | | | 336g, 337Δ, 337a, 337c, 337t, 337g, 338t, 338g, 338a, 338Δ, 338c, 339a, 339t, 339Δ, 339c, 339g, 340g, 340c, 340a, 340Δ, 340t, 341g, |
| | | | 341t, 341c, 341a, 341Δ, 342c, 342t, 342g, 342Δ, 342a, 343t, 343g, 343Δ, 343c, 343a, 344g, 344c, 344t, 344a, 344Δ, 345g, 345Δ, 345t, |
| | | | 345c, 345a, 346t, 346Δ, 346g, 346a, 346c, 347g, 347t, 347a, 347c, 347Δ, 348g, 348a, 348t, 348c, 348Δ, 349g, 349c, 349t, 349a, 349Δ, |
| | | | 350g, 350Δ, 350t, 350a, 350c, 351a, 351Δ, 351c, 351t, 351g, 352Δ, 352c, 352g, 352a, 352t, 353c, 353g, 353a, 353t, 353Δ, 354c, 354g, |
| | | | 354a, 354t, 354Δ, 355g, 355t, 355a, 355c, 355Δ, 356g, 356t, 356a, 356c, 356Δ, 357t, 357a, 357c, 357g, 357Δ, 358c, 358g, 358t, 358a, |
| | | | 358Δ, 359g, 359t, 359c, 359a, 359Δ, 360g, 360t, 360a, 360c, 360Δ, 361a, 361t, 361g, 361c, 361Δ, 362t, 362a, 362c, 362g, 362Δ, 363a, |
| | | | 363g, 363t, 363Δ, 363c, 364c, 364t, 364a, 364g, 364Δ, 365c, 365a, 365t, 365g, 365Δ, 366t, 366g, 366c, 366a, 366Δ, 367t, 367g, 367c, |
| | | | 367Δ, 367a, 368t, 368c, 368g, 368a, 368Δ, 369g, 369c, 369Δ, 369a, 369t, 370g, 370t, 370a, 370c, 370Δ, 371c, 371a, 371Δ, 371g, 371t, |
| | | | 372t, 372g, 372c, 372a, 372Δ, 373c, 373g, 373t, 373Δ, 373a, 374g, 374t, 374a, 374Δ, 374c, 375t, 375c, 375a, 375g, 375Δ, 376c, 376a, 376t, |
| | | | 376g, 376Δ, 377a, 377Δ, 377t, 377c, 377g, 378a, 378t, 378g, 378c, 378Δ, 379a, 379c, 379g, 379t, 379Δ, 380g, 380c, 380a, 380t, 380Δ, |
| | | | 381t, 381a, 381c, 381Δ, 381g, 382t, 382a, 382g, 382Δ, 382c, 383a, 383t, 383c, 383g, 383Δ, 384Δ, 384t, 384a, 384g, 384c, 385g, 385c, |
| | | | 385a, 385t, 385Δ, 386Δ, 386a, 386t, 386g, 386c, 387c, 387g, 387t, 387a, 387Δ, 388t, 388a, 388Δ, 388g, 388c, 389t, 389g, 389a, 389c, |
| | | | 389Δ, 390a, 390Δ, 390c, 390t, 390g, 391t, 391g, 391a, 391Δ, 391c, 392g, 392a, 392c, 392t, 392Δ, 393t, 393g, 393a, 393c, 393Δ, 394g, |
| | | | 394t, 394a, 394c, 395a, 395t, 395c, 395g, 395Δ, 396t, 396a, 396c, 396g, 396Δ, 397a, 397g, 397Δ, 397c, 397t, 398c, 398t, 398g, 398a, 398Δ, |
| | | | 399g, 399a, 399c, 399t, 400a, 400c, 400g, 400t, 400Δ, 401c, 401g, 401t, 401a, 401Δ, 402a, 402c, 402t, 402g, 402Δ, 403c, 403g, 403a, 403t, |
| | | | 403Δ, 404t, 404g, 404a, 404c, 404Δ, 405c, 405g, 405a, 405t, 405Δ, 406t, 406c, 406g, 406a, 406Δ, 407a, 407c, 407g, 407t, 407Δ, 408c, |
| | | | 408g, 408t, 408a, 408Δ, 409a, 409g, 409Δ, 409t, 409c, 410g, 410a, 410t, 410c, 410Δ, 411g, 411t, 411a, 411c, 411Δ, 412a, 412g, 412t, 412c, |
| | | | 412Δ, 413a, 413g, 413c, 413t, 413Δ, 414t, 414g, 414c, 414a, 414Δ, 415a, 415g, 415t, 415c, 415Δ, 416a, 416g, 416t, 416Δ, 416c, 417g, |
| | | | 417a, 417t, 417Δ, 417c, 418a, 418g, 418c, 418Δ, 418t, 419t, 419c, 419Δ, 419a, 419g, 420c, 420g, 420a, 420t, 420Δ, 421c, 421a, 421g, |
| | | | 421Δ, 422a, 422c, 422g, 422t, 422Δ, 423a, 423c, 423Δ, 423t, 423g, 424t, 424a, 424g, 424Δ, 424c, 425g, 425c, 425a, 425t, 425Δ, 426c, |
| | | | 426a, 426g, 426Δ, 426t, 427c, 427g, 427a, 427Δ, 427t, 428a, 428c, 428g, 428Δ, 428t, 429g, 429t, 429Δ, 429a, 429c, 430g, 430a, 430Δ, |
| | | | 430c, 430t, 431a, 431g, 431t, 431c, 431Δ, 432g, 432a, 432t, 432c, 433t, 433a, 433g, 433c, 433Δ, 434t, 434a, 434g, 434Δ, 434c, 435a, 435g, |
| | | | 435c, 435t, 435Δ, 436g, 436a, 436c, 436t, 436Δ, 437g, 437a, 437t, 438a, 438c, 438g, 438Δ, 438t, 439a, 439t, 439c, 439g, 439Δ, 440g, 440c, |
| | | | 440a, 440t, 440Δ, 441a, 441t, 441c, 441Δ, 441g, 442g, 442t, 442c, 442a, 443t, 443g, 443a, 443Δ, 443c, 444a, 444g, 444Δ, 444t, 444c, 445t, |
| | | | 445g, 445Δ, 445c, 445a, 446g, 446a, 446t, 446Δ, 446c, 447g, 447t, 447a, 447c, 447Δ, 448c, 448a, 448t, 448g, 449c, 449a, 449g, 449t, |
| | | | 449Δ, 450a, 450t, 450c, 450g, 451g, 451a, 451c, 451t, 451Δ, 452a, 452g, 452t, 452c, 452Δ, 453g, 453t, 453a, 453c, 453Δ, 454t, 454a, 454g, |
| | | | 454c, 454Δ, 455g, 455c, 455a, 455Δ, 455t, 456a, 456c, 456t, 456g, 456Δ, 457a, 457g, 457Δ, 457t, 457c, 458g, 458t, 458Δ, 458a, 458c, |
| | | | 459t, 459g, 459a, 459c, 460g, 460t, 460Δ, 460a, 460c, 461g, 461t, 461a, 461c, 461Δ, 462c, 462a, 462Δ, 462t, 462g, 463t, 463g, 463c, 463Δ, |
| | | | 463a, 464a, 464t, 464Δ, 464c, 464g, 465c, 465t, 465g, 465Δ, 465a, 466c, 466a, 466Δ, 466g, 466t, 467a, 467c, 467t, 467Δ, 467g, 468t, 468a, |
| | | | 468c, 468Δ, 468g, 469a, 469t, 469c, 469Δ, 469g, 470g, 470t, 470Δ, 470c, 470a, 471t, 471c, 471Δ, 471a, 471g, 472Δ, 472c, 472a, 472g, |
| | | | 472t, 473a, 473c, 473t, 473g, 473Δ, 474t, 474a, 474c, 474g, 474Δ, 475t, 475a, 475g, 475c, 475Δ, 476a, 476t, 476g, 476c, 476Δ, 477g, 477a, |
| | | | 477c, 477t, 478t, 478c, 478Δ, 478g, 478a, 479a, 479g, 479c, 479Δ, 479t, 480g, 480Δ, 480c, 480t, 480a, 481t, 481g, 481a, 481c, 481Δ, 482t, |
| | | | 482g, 482Δ, 482c, 482a, 483g, 483a, 483Δ, 483t, 483c, 484c, 484Δ, 484g, 484a, 485g, 485c, 485a, 485t, 485Δ, 486t, 486a, 486c, 486Δ, |
| | | | 486g, 487t, 487g, 487Δ, 487c, 487a, 488c, 488a, 488g, 488t, 488Δ, 489a, 489g, 489c, 489Δ, 489t, 490g, 490a, 490c, 490t, 490Δ, 491Δ, |
| | | | 491g, 491t, 491a, 491c, 492t, 492a, 492g, 492c, 492Δ, 493c, 493a, 493t, 493g, 494a, 494c, 494g, 494Δ, 494t, 495c, 495a, 495Δ, 495t, 495g, |
| | | | 496t, 496g, 496Δ, 496a, 497t, 497c, 497g, 497a, 497Δ, 498Δ, 498a, 498t, 499Δ, 499t, 499g, 499a, 500a, 500t, 500c, 500Δ, 501Δ, 501g, |
| | | | 501c, 501t, 501a, 502a, 502t, 502g, 502Δ, 502c, 503g, 503a, 503t, 503Δ, 503c, 504t, 504g, 504Δ, 504c, 504a, 505t, 505g, 505a, 505Δ, 505c, |
| | | | 506t, 506c, 506Δ, 506g, 506a, 507Δ, 507t, 507g, 507c, 507a, 508g, 508Δ, 508t, 508a, 508c, 509Δ, 509a, 509t, 509c, 509g, 510Δ, 510c, |
| | | | 510t, 510g, 510a, 511t, 511Δ, 511a, 511c, 511g, 512t, 512Δ, 512g, 512a, 513Δ, 513a, 513t, 513g, 513c, 514a, 514Δ, 514g, 514t, 514c, 515a, |
| | | | 515Δ, 515t, 515c, 515g, 516t, 516Δ, 516a, 516g, 516c, 517t, 517a, 517g, 517Δ, 518a, 518t, 518Δ, 518g, 518c, 519Δ, 519g, 519t, 519a, |
| | | | 519c, 520a, 520Δ, 520g, 520c, 521t, 521Δ, 521g, 521a, 521c, 522Δ, 522g, 522t, 522c, 522a, 523t, 523Δ, 523a, 523g, 523c, 524g, 524Δ, |
| | | | 524t, 524c, 524a, 525g, 525a, 525Δ, 525t, 525c, 526g, 526Δ, 526a, 526t, 527Δ, 527t, 527g, 527c, 528Δ, 528a, 528c, 528t, 528g, 529Δ, |
| | | | 529c, 529t, 529g, 529a, 530Δ, 530g, 531a, 531t, 531Δ, 532c, 532g, 532Δ, 533a, 533g, 533c, 533t, 534c, 534g, 534a, 535Δ, 535c, 535g, |
| | | | 535a, 536a, 536t, 536g, 536c, 537g, 537t, 537c, 537a, 538a, 538g, 538c, 539g, 539Δ, 539a, 539t, 539c, 540g, 540c, 540Δ, 540a, 540t, 541c, |
| | | | 541t, 541g, 541a, 542g, 542c, 542a, 543a, 543g, 543c, 544g, 544c, 544a, 545g, 545c, 545a, 546c, 546g, 546a, 547g, 547a, 547c, 547t, 548c, |
| | | | 548g, 548a, 548t, 549g, 549c, 549a, 550g, 550a, 550t, 550Δ, 550c, 551c, 551a, 551g, 551t, 552a, 552g, 552t, 552Δ, 552c, 553c, 553g, 553a, |
| | | | 553Δ, 554a, 554g, 554Δ, 555t, 555g, 555c, 555a, 555Δ, 556a, 556g, 556c, 556Δ, 556t, 557c, 557g, 557a, 557Δ, 557t, 558c, 558g, 558t, |
| | | | 558a, 559a, 559c, 559t, 559Δ, 559g, 560a, 560g, 560t, 560Δ, 560c, 561a, 561t, 561c, 561Δ, 562a, 562g, 562t, 563t, 563c, 563a, 563g, 563Δ, |
| | | | 564t, 564a, 564c, 564Δ, 564g, 565c, 565a, 565t, 565Δ, 565g, 566c, 566a, 566t, 566g, 566Δ, 567a, 567g, 567t, 567Δ, 568t, 568g, 568c, 568a, |
| | | | 568Δ, 569c, 569g, 569Δ, 569a, 569t, 570g, 570Δ, 570c, 570t, 571Δ, 571g, 571a, 571t, 572a, 572t, 572g, 572c, 572Δ, 573a, 573t, 573c, |
| | | | 573g, 574g, 574a, 574Δ, 574t, 575g, 575a, 575Δ, 575t, 576g, 576Δ, 576t, 576c, 576a, 577t, 577Δ, 577g, 577a, 577c, 578g, 578a, 578Δ, |
| | | | 578t, 579t, 579g, 579a, 579Δ, 579c, 580g, 580a, 580t, 580c, 580Δ, 581a, 581g, 581c, 581Δ, 581t, 582Δ, 582c, 582a, 582g, 583t, 583a, |
| | | | 583Δ, 584t, 584g, 585t, 585g, 585c, 586a, 587a, 587g, 587c, 587t, 588g, 588t, 590c, 591t, 591a, 592g, 592t, 592c, 593t, 593a, 593g, 593c, |
| | | | 594c, 594t, 595a, 595c, 595t, 596c, 596t, 596a, 597t, 597a, 598c, 598t, 599c, 599t, 599a, 600a, 600c, 601a, 602c, 602Δ, 602a, 603c, 603t, |
| | | | 604t, 604g, 604a, 605g, 606g, 3_4ins_g, 4_5ins_c, 4_5ins_g, 5_6ins_n, 5_6ins_t, 5_6ins_c, 6_7ins_a, 6_7ins_c, 6_7ins_t, 6_7ins_r, |
| | | | 6_7ins_g, 7_8ins_t, 7_8ins_c, 7_8ins_a, 7_8ins_g, 7_8ins_m, 7_8ins_n, 8_9ins_c, 8_9ins_t, 8_9ins_g, 8_9ins_a, 9_10ins_t, 9_10ins_a, |
| | | | 9_10ins_c, 9_10ins_g, 9_10ins_n, 10_11ins_t, 10_11ins_c, 10_11ins_a, 10_11ins_n, 11_12ins_c, 11_12ins_a, 11_12ins_t, 11_12ins_g, |
| | | | 12_13ins_t, 12_13ins_c, 12_13ins_a, 13_14ins_c, 13_14ins_t, 13_14ins_a, 13_14ins_g, 14_15ins_c, 14_15ins_t, 14_15ins_g, 14_15ins_a, |
| | | | 15_16ins_c, 15_16ins_t, 15_16ins_a, 15_16ins_g, 16_17ins_c, 16_17ins_a, 16_17ins_t, 16_17ins_g, 17_18ins_g, 17_18ins_t, 17_18ins_c, |
| | | | 17_18ins_a, 18_19ins_t, 18_19ins_a, 18_19ins_g, 18_19ins_c, 19_20ins_g, 19_20ins_a, 19_20ins_c, 19_20ins_t, 20_21ins_g, 20_21ins_c, |
| | | | 20_21ins_t, 21_22ins_t, 21_22ins_a, 21_22ins_c, 21_22ins_g, 22_23ins_t, 22_23ins_a, 22_23ins_g, 22_23ins_c, 23_24ins_a, 23_24ins_n, |
| | | | 23_24ins_g, 23_24ins_c, 23_24ins_t, 24_25ins_c, 24_25ins_t, 24_25ins_g, 24_25ins_a, 25_26ins_n, 25_26ins_a, 25_26ins_c, 25_26ins_t, |
| | | | 25_26ins_g, 26_27ins_g, 26_27ins_a, 26_27ins_c, 26_27ins_t, 27_28ins_c, 27_28ins_g, 27_28ins_a, 28_29ins_t, 28_29ins_g, |
| | | | 28_29ins_c, 29_30ins_c, 29_30ins_g, 29_30ins_t, 29_30ins_a, 30_31ins_g, 30_31ins_t, 30_31ins_c, 30_31ins_a, 31_32ins_a, 31_32ins_t, |
| | | | 31_32ins_c, 31_32ins_g, 32_33ins_a, 32_33ins_t, 32_33ins_c, 32_33ins_g, 33_34ins_a, 33_34ins_t, 33_34ins_c, 33_34ins_g, 34_35ins_a, |
| | | | 34_35ins_g, 34_35ins_t, 34_35ins_c, 35_36ins_a, 35_36ins_g, 35_36ins_c, 35_36ins_t, 36_37ins_c, 36_37ins_t, 36_37ins_g, 36_37ins_a, |
| | | | 37_38ins_a, 37_38ins_c, 37_38ins_t, 37_38ins_g, 38_39ins_g, 38_39ins_t, 38_39ins_a, 38_39ins_c, 39_40ins_t, 39_40ins_a, 39_40ins_g, |
| | | | 39_40ins_c, 40_41ins_a, 40_41ins_g, 40_41ins_c, 40_41ins_t, 41_42ins_a, 41_42ins_t, 41_42ins_c, 41_42ins_g, 41_42ins_n, 42_43ins_a, |
| | | | 42_43ins_g, 42_43ins_t, 42_43ins_c, 43_44ins_g, 43_44ins_t, 43_44ins_a, 44_45ins_a, 44_45ins_g, 44_45ins_t, 44_45ins_n, 45_46ins_g, |
| | | | 45_46ins_a, 45_46ins_c, 45_46ins_t, 46_47ins_t, 47_48ins_g, 47_48ins_c, 47_48ins_t, 47_48ins_a, 48_49ins_t, 48_49ins_c, 48_49ins_a, |
| | | | 48_49ins_g, 49_50ins_t, 49_50ins_a, 49_50ins_y, 49_50ins_c, 49_50ins_g, 50_51ins_t, 50_51ins_a, 50_51ins_c, 50_51ins_g, |
| | | | 51_52ins_g, 51_52ins_c, 51_52ins_t, 51_52ins_a, 52_53ins_t, 52_53ins_g, 52_53ins_a, 52_53ins_c, 53_54ins_a, 53_54ins_t, 53_54ins_c, |
| | | | 53_54ins_g, 54_55ins_c, 54_55ins_t, 54_55ins_g, 54_55ins_a, 55_56ins_t, 55_56ins_c, 55_56ins_g, 55_56ins_a, 56_57ins_a, |
| | | | 56_57ins_n, 56_57ins_t, 56_57ins_c, 56_57ins_g, 57_58ins_a, 57_58ins_t, 57_58ins_c, 57_58ins_g, 58_59ins_g, 58_59ins_c, |
| | | | 58_59ins_a, 58_59ins_n, 59_60ins_c, 59_60ins_g, 59_60ins_a, 59_60ins_n, 60_61ins_g, 60_61ins_n, 60_61ins_a, 60_61ins_t, |
| | | | 60_61ins_c, 61_62ins_n, 61_62ins_c, 61_62ins_a, 61_62ins_t, 62_63ins_c, 62_63ins_t, 62_63ins_n, 62_63ins_a, 62_63ins_g, 63_64ins_t, |
| | | | 63_64ins_n, 63_64ins_a, 63_64ins_c, 64_65ins_t, 64_65ins_a, 64_65ins_c, 64_65ins_g, 65_66ins_n, 65_66ins_g, 65_66ins_a, |
| | | | 65_66ins_c, 65_66ins_w, 66_67ins_c, 66_67ins_t, 66_67ins_a, 66_67ins_g, 67_68ins_t, 67_68ins_c, 67_68ins_g, 67_68ins_a, |
| | | | 68_69ins_g, 68_69ins_a, 68_69ins_c, 68_69ins_t, 69_70ins_a, 69_70ins_g, 69_70ins_t, 69_70ins_c, 70_71ins_g, 70_71ins_a, 70_71ins_t, |
| | | | 70_71ins_c, 71_72ins_g, 71_72ins_a, 71_72ins_t, 71_72ins_c, 72_73ins_t, 72_73ins_c, 72_73ins_a, 72_73ins_g, 73_74ins_a, 73_74ins_c, |
| | | | 73_74ins_t, 73_74ins_g, 74_75ins_c, 74_75ins_t, 74_75ins_a, 74_75ins_g, 75_76ins_t, 75_76ins_a, 75_76ins_g, 75_76ins_c, 76_77ins_t, |
| | | | 76_77ins_g, 76_77ins_c, 76_77ins_a, 77_78ins_c, 77_78ins_t, 77_78ins_g, 77_78ins_a, 78_79ins_a, 78_79ins_c, 78_79ins_t, 78_79ins_g, |
| | | | 79_80ins_c, 79_80ins_a, 79_80ins_g, 79_80ins_t, 80_81ins_a, 80_81ins_t, 80_81ins_c, 80_81ins_g, 81_82ins_c, 81_82ins_g, 81_82ins_t, |
| | | | 82_83ins_t, 82_83ins_c, 82_83ins_g, 82_83ins_a, 83_84ins_a, 83_84ins_t, 83_84ins_c, 83_84ins_g, 84_85ins_c, 84_85ins_a, 84_85ins_g, |
| | | | 84_85ins_t, 85_86ins_s, 85_86ins_a, 85_86ins_t, 86_87ins_g, 86_87ins_a, 87_88ins_g, 87_88ins_c, 88_89ins_c, 88_89ins_t, 88_89ins_g, |
| | | | 89_90ins_a, 89_90ins_n, 89_90ins_c, 90_91ins_a, 90_91ins_c, 90_91ins_g, 91_92ins_c, 91_92ins_g, 91_92ins_t, 91_92ins_a, 92_93ins_t, |
| | | | 92_93ins_a, 92_93ins_c, 93_94ins_g, 93_94ins_t, 93_94ins_a, 95_96ins_t, 95_96ins_c, 95_96ins_g, 96_97ins_c, 96_97ins_g, 96_97ins_t, |
| | | | 96_97ins_a, 97_98ins_a, 97_98ins_t, 97_98ins_c, 97_98ins_g, 98_99ins_t, 98_99ins_g, 98_99ins_c, 98_99ins_a, 99_100ins_n, |
| | | | 99_100ins_t, 99_100ins_a, 99_100ins_c, 100_101ins_a, 100_101ins_t, 100_101ins_c, 101_102ins_t, 101_102ins_a, 101_102ins_c, |
| | | | 102_103ins_t, 102_103ins_c, 102_103ins_n, 102_103ins_g, 103_104ins_c, 103_104ins_t, 103_104ins_g, 103_104ins_a, 104_105ins_g, |
| | | | 104_105ins_c, 104_105ins_a, 104_105ins_t, 105_106ins_a, 105_106ins_c, 105_106ins_g, 105_106ins_t, 106_107ins_c, 106_107ins_g, |
| | | | 106_107ins_a, 106_107ins_w, 107_108ins_a, 107_108ins_g, 107_108ins_c, 108_109ins_c, 108_109ins_a, 108_109ins_g, 109_110ins_a, |
| | | | 109_110ins_c, 109_110ins_t, 109_110ins_g, 110_111ins_a, 110_111ins_c, 110_111ins_t, 110_111ins_g, 110_111ins_n, 111_112ins_g, |
| | | | 111_112ins_t, 111_112ins_c, 111_112ins_a, 112_113ins_c, 112_113ins_a, 112_113ins_g, 113_114ins_g, 113_114ins_c, 113_114ins_a, |
| | | | 113_114ins_t, 114_115ins_a, 114_115ins_g, 114_115ins_c, 114_115ins_t, 115_116ins_g, 115_116ins_a, 115_116ins_c, 115_116ins_t, |
| | | | 117_118ins_t, 117_118ins_a, 117_118ins_g, 118_119ins_c, 118_119ins_a, 118_119ins_t, 118_119ins_g, 119_120ins_g, 119_120ins_c, |
| | | | 119_120ins_a, 119_120ins_t, 120_121ins_t, 120_121ins_a, 120_121ins_c, 120_121ins_g, 121_122ins_t, 121_122ins_g, 121_122ins_a, |
| | | | 121_122ins_c, 122_123ins_g, 122_123ins_c, 122_123ins_h, 123_124ins_a, 123_124ins_t, 123_124ins_c, 124_125ins_c, 124_125ins_t, |
| | | | 124_125ins_a, 124_125ins_g, 125_126ins_c, 125_126ins_a, 125_126ins_t, 125_126ins_g, 126_127ins_g, 126_127ins_a, 126_127ins_t, |
| | | | 126_127ins_c, 127_128ins_t, 127_128ins_c, 127_128ins_g, 127_128ins_a, 128_129ins_a, 128_129ins_g, 128_129ins_c, 129_130ins_c, |
| | | | 129_130ins_a, 129_130ins_g, 129_130ins_t, 130_131ins_t, 130_131ins_g, 130_131ins_a, 130_131ins_c, 131_132ins_g, 131_132ins_a, |
| | | | 131_132ins_c, 131_132ins_n, 131_132ins_t, 132_133ins_t, 132_133ins_g, 132_133ins_c, 132_133ins_a, 133_134ins_c, 133_134ins_t, |
| | | | 133_134ins_a, 133_134ins_g, 134_135ins_c, 134_135ins_t, 134_135ins_g, 134_135ins_a, 135_136ins_a, 135_136ins_t, 135_136ins_g, |
| | | | 135_136ins_c, 136_137ins_c, 136_137ins_g, 136_137ins_t, 137_138ins_g, 137_138ins_a, 137_138ins_c, 138_139ins_t, 138_139ins_g, |
| | | | 138_139ins_c, 139_140ins_g, 139_140ins_t, 139_140ins_c, 139_140ins_a, 140_141ins_t, 140_141ins_c, 140_141ins_g, 140_141ins_n, |
| | | | 141_142ins_t, 141_142ins_a, 141_142ins_c, 142_143ins_a, 142_143ins_t, 142_143ins_c, 142_143ins_g, 143_144ins_a, 143_144ins_g, |
| | | | 143_144ins_c, 144_145ins_a, 144_145ins_c, 144_145ins_g, 144_145ins_t, 145_146ins_t, 145_146ins_c, 145_146ins_a, 145_146ins_g, |
| | | | 146_147ins_g, 146_147ins_c, 146_147ins_t, 147_148ins_g, 147_148ins_t, 147_148ins_a, 147_148ins_c, 148_149ins_t, 148_149ins_c, |
| | | | 148_149ins_g, 148_149ins_a, 149_150ins_t, 149_150ins_a, 149_150ins_c, 149_150ins_g, 150_151ins_a, 150_15lins_c, 150_151ins_g, |
| | | | 150_151ins_t, 151_152ins_a, 151_152ins_g, 151_152ins_t, 151_152ins_c, 152_153ins_t, 152_153ins_a, 152_153ins_g, 152_153ins_c, |
| | | | 153_154ins_t, 153_154ins_a, 153_154ins_g, 153_154ins_c, 154_155ins_t, 154_155ins_a, 154_155ins_g, 154_155ins_c, 155_156ins_g, |
| | | | 155_156ins_t, 155_156ins_c, 155_156ins_a, 156_157ins_t, 156_157ins_g, 156_157ins_a, 156_157ins_c, 157_158ins_c, 157_158ins_a, |
| | | | 157_158ins_g, 157_158ins_t, 158_159ins_g, 158 159ins_a, 158_159ins_c, 158_159ins_t, 159_160ins_g, 159_160ins_t, 159_160ins_a, |
| | | | 159_160ins_c, 160_161ins_c, 160_161ins_g, 160_161ins_t, 160_161ins_a, 160_161ins_r, 161_162ins_g, 161_162ins_t, 161_162ins_a, |
| | | | 161_162ins_c, 162_163ins_t, 162_163ins_c, 162_163ins_a, 162_163ins_g, 163_164ins_g, 163_164ins_t, 163_164ins_a, 163_164ins_c, |
| | | | 164_165ins_g, 164_165ins_t, 164_165ins_c, 164_165ins_a, 165_166ins_a, 165_166ins_c, 165_166ins_t, 165_166ins_g, 165_166ins_y, |
| | | | 166_167ins_t, 166_167ins_a, 166_167ins_g, 166_167ins_c, 167_168ins_a, 167_168ins_t, 167_168ins_c, 167_168ins_g, 168_169ins_a, |
| | | | 168_169ins_c, 168_169ins_t, 168_169ins_g, 169_170ins_a, 169_170ins_g, 169_170ins_t, 169_170ins_n, 169_170ins_c, 170_171ins_t, |
| | | | 170_171ins_a, 170_171ins_g, 170_171ins_n, 170_171ins_c, 170_171ins_s, 171_172ins_t, 171_172ins_g, 171_172ins_a, 171_172ins_c, |
| | | | 172_173ins_g, 172_173ins_a, 172_173ins_t, 172_173ins_c, 172_173ins_r, 173_174ins_t, 173_174ins_a, 173_174ins_g, 173_174ins_c, |
| | | | 174_175ins_a, 174_175ins_t, 174_175ins_c, 174_175ins_g, 175_176ins_a, 175_176ins_t, 175_176ins_g, 175_176ins_c, 176_177ins_a, |
| | | | 176_177ins_g, 176_177ins_t, 176_177ins_c, 177_178ins_a, 177_178ins_t, 177_178ins_g, 178_179ins_t, 178_179ins_a, 178_179ins_g, |
| | | | 178_179ins_c, 179_180ins_a, 179_180ins_t, 179_180ins_g, 179_180ins_c, 180_181ins_a, 180_181ins_c, 180_181ins_t, 181_182ins_a, |
| | | | 181_182ins_c, 181_182ins_t, 181_182ins_g, 182_183ins_a, 182_183ins_c, 182_183ins_g, 183_184ins_t, 183_184ins_g, 184_185ins_t, |
| | | | 184_185ins_c, 184_185ins_a, 184_185ins_g, 185_186ins_a, 185_186ins_c, 185_186ins_t, 185_186ins_g, 186_187ins_a, 186_187ins_c, |
| | | | 186_187ins_t, 186_187ins_g, 187_188ins_t, 187_188ins_g, 187_188ins_a, 187_188ins_c, 188_189ins_c, 188_189ins_g, 188_189ins_t, |
| | | | 189_190ins_g, 189_190ins_c, 189_190ins_t, 189_190ins_a, 190_191ins_a, 190_191ins_c, 190_191ins_t, 190_191ins_g, 191_192ins_c, |
| | | | 191_192ins_a, 191_192ins_t, 191_192ins_g, 192_193ins_c, 192_193ins_t, 192_193ins_a, 193_194ins_t, 193_194ins_a, 193_194ins_g, |
| | | | 193_194ins_c, 194_195ins_g, 194_195ins_n, 194_195ins_c, 195_196ins_c, 195_196ins_t, 195_196ins_g, 195_196ins_a, 196_197ins_t, |
| | | | 196_197ins_c, 196_197ins_g, 196_197ins_a, 197_198ins_c, 197_198ins_g, 198_199ins_g, 199_200ins_a, 199_200ins_g, 199_200ins_t, |
| | | | 200_201ins_t, 200_201ins_a, 200_201ins_g, 201_202ins_t, 201_202ins_a, 201_202ins_c, 201_202ins_g, 202_203ins_c, 202_203ins_g, |
| | | | 202_203ins_a, 202_203ins_t, 203_204ins_g, 203_204ins_t, 203_204ins_c, 204_205ins_c, 204_205ins_t, 204_205ins_g, 204_205ins_a, |
| | | | 205_206ins_g, 205_206ins_t, 205_206ins_a, 205_206ins_c, 206_207ins_c, 206_207ins_t, 206_207ins_a, 206_207ins_g, 207_208ins_g, |
| | | | 207_208ins_t, 207_208ins_a, 208_209ins_t, 208_209ins_c, 208_209ins_g, 208_209ins_a, 208_209ins_n, 209_210ins_a, 209_210ins_g, |
| | | | 209_210ins_c, 209_210ins_t, 210_211ins_a, 210_211ins_t, 210_211ins_g, 210_211ins_c, 211_212ins_a, 211_212ins_g, 211_212ins_t, |
| | | | 211_212ins_n, 211_212ins_c, 212_213ins_a, 212_213ins_t, 212_213ins_c, 212_213ins_g, 213_214ins_c, 213_214ins_a, 213_214ins_t, |
| | | | 213_214ins_m, 213_214ins_g, 213_214ins_r, 214_215ins_g, 214_215ins_a, 214_215ins_t, 214_215ins_c, 215_216ins_a, 215_216ins_c, |
| | | | 215_216ins_g, 215_216ins_t, 216_217ins_g, 216_217ins_a, 216_217ins_t, 216_217ins_c, 217_218ins_g, 217_218ins_a, 217_218ins_t, |
| | | | 218_219ins_a, 218_219ins_t, 218_219ins_g, 218_219ins_c, 219_220ins_a, 219_220ins_c, 219_220ins_t, 219_220ins_g, 220_221ins_t, |
| | | | 220_221ins_a, 220_221ins_g, 220_221ins_c, 221_222ins_t, 221_222ins_g, 221_222ins_c, 221_222ins_a, 222_223ins_g, 222_223ins_a, |
| | | | 222_223ins_t, 223_224ins_a, 223_224ins_c, 223_224ins_t, 223_224ins_g, 224_225ins_a, 224_225ins_c, 224_225ins_g, 224_225ins_t, |
| | | | 225_226ins_t, 225_226ins_a, 225_226ins_c, 225_226ins_g, 226_227ins_g, 226_227ins_t, 226_227ins_a, 226_227ins_c, 227_228ins_t, |
| | | | 227_228ins_g, 227_228ins_c, 227_228ins_a, 228_229ins_g, 228_229ins_t, 228_229ins_c, 228_229ins_a, 229_230ins_t, 229_230ins_a, |
| | | | 229_230ins_g, 229_230ins_c, 230_231ins_a, 230_231ins_g, 230_231ins_t, 230_231ins_c, 231_232ins_a, 231_232ins_g, 231_232ins_c, |
| | | | 231_232ins_t, 232_233ins_t, 232_233ins_a, 232_233ins_c, 232_233ins_g, 233_234ins_a, 233_234ins_g, 233_234ins_c, 233_234ins_t, |
| | | | 234_235ins_t, 234_235ins_a, 234_235ins_g, 234_235ins_c, 235_236ins_a, 235_236ins_g, 235_236ins_t, 235_236ins_c, 236_237ins_a, |
| | | | 236_237ins_g, 236_237ins_t, 236_237ins_c, 237_238ins_a, 237_238ins_c, 237_238ins_g, 237_238ins_t, 238_239ins_a, 238_239ins_c, |
| | | | 238_239ins_t, 238_239ins_g, 239_240ins_g, 239_240ins_a, 239_240ins_t, 239_240ins_c, 240_241ins_g, 240_241ins_c, 240_241ins_a, |
| | | | 240_241ins_t, 241_242ins_t, 241_242ins_a, 241_242ins_g, 241_242ins_c, 242_243ins_a, 242_243ins_c, 242_243ins_t, 242_243ins_g, |
| | | | 243_244ins_t, 243_244ins_a, 243_244ins_c, 243_244ins_g, 244_245ins_g, 244_245ins_t, 244_245ins_c, 244_245ins_a, 245_246ins_g, |
| | | | 245_246ins_t, 245_246ins_a, 245_246ins_c, 246_247ins_g, 246_247ins_t, 246_247ins_a, 246_247ins_c, 247_248ins_c, 247_248ins_a, |
| | | | 247_248ins_g, 247_248ins_t, 248_249ins_a, 248_249ins_t, 248_249ins_g, 248_249ins_c, 249_250ins_a, 249_250ins_t, 249_250ins_c, |
| | | | 249_250ins_g, 250_251ins_t, 250_251ins_c, 250_251ins_a, 250_251ins_g, 251_252ins_c, 251_252ins_a, 251_252ins_t, 251_252ins_g, |
| | | | 252_253ins_t, 252_253ins_a, 252_253ins_c, 252_253ins_g, 253_254ins_t, 253_254ins_a, 253_254ins_g, 253_254ins_c, 254_255ins_t, |
| | | | 254_255ins_a, 254_255ins_c, 254_255ins_g, 255_256ins_t, 255_256ins_a, 255_256ins_g, 255_256ins_c, 256_257ins_a, 256_257ins_c, |
| | | | 256_257ins_g, 256_257ins_t, 257_258ins_a, 257_258ins_c, 257_258ins_t, 257_258ins_g, 258_259ins_a, 258_259ins_c, 258_259ins_g, |
| | | | 258_259ins_t, 259_260ins_g, 259_260ins_c, 259_260ins_t, 259_260ins_a, 260_261ins_c, 260_261ins_a, 260_261ins_n, 260_261ins_g, |
| | | | 260_261ins_t, 261_262ins_g, 261_262ins_c, 261_262ins_t, 261_262ins_a, 262_263ins_a, 262_263ins_c, 262_263ins_g, 262_263ins_t, |
| | | | 263_264ins_a, 263_264ins_t, 263_264ins_c, 263_264ins_g, 263_264ins_w, 264_265ins_t, 264_265ins_a, 264_265ins_c, 264_265ins_g, |
| | | | 265_266ins_a, 265_266ins_c, 265_266ins_t, 265_266ins_g, 266_267ins_t, 266_267ins_c, 266_267ins_m, 266_267ins_g, 266_267ins_a |
| 243 | antis_5p_EVD68 | 6 | 5t, 7a, 8g, 35t, 25_26ins_a, 25_26ins_g |
| 245 | antis_5p_NDV | 251 | 5g, 9g, 11g, 11a, 12g, 14g, 15g, 16g, 21g, 22a, 22g, 23a, 23g, 23c, 24g, 24Δ, 25g, 25Δ, 26g, 26Δ, 26c, 27g, 27Δ, 29Δ, 29g, 30Δ, 30t, 32Δ, |
| | | | 32t, 32g, 33t, 34a, 34g, 35t, 35a, 35g, 37t, 39g, 41g, 42g, 42c, 43c, 43g, 46c, 47c, 47a, 48a, 48t, 48c, 49a, 49c, 50a, 50t, 51g, 51t, 52a, 52t, |
| | | | 53t, 54g, 54Δ, 55Δ, 55c, 57t, 58a, 58c, 59c, 59a, 61t, 61g, 61c, 62c, 63a, 63c, 64a, 64g, 65t, 65g, 67t, 67a, 68a, 68c, 68t, 69t, 71g, 71a, 72t, |
| | | | 72c, 73c, 73g, 74g, 74c, 75g, 75a, 75c, 76t, 77c, 79t, 79c, 79a, 79g, 80c, 80a, 81t, 81g, 81c, 82g, 82c, 82t, 83c, 85t, 85g, 86g, 86t, 87c, 87t, |
| | | | 87a, 88t, 88c, 88a, 88g, 90t, 90g, 91t, 91c, 92a, 92t, 92c, 93t, 93c, 93g, 93Δ, 94c, 95g, 95a, 95t, 96g, 96a, 96c, 97a, 97g, 99a, 101g, 101Δ, |
| | | | 102g, 102Δ, 102a, 102t, 103a, 103c, 103Δ, 104g, 104a, 104t, 104Δ, 105g, 106g, 106a, 107g, 107a, 108a, 108t, 110g, 110t, 111a, 111t, 112a, |
| | | | 112g, 113c, 113t, 114t, 114a, 115a, 116a, 116g, 117a, 119g, 121g, 122a, 122g, 123g, 123a, 124a, 124t, 125t, 126a, 126g, 127a, 127c, 127g, |
| | | | 128a, 128g, 129a, 130g, 130a, 131g, 131a, 131c, 132g, 132a, 132c, 132t, 133g, 133a, 133c, 133t, 134g, 134a, 135c, 136a, 136c, 137a, 137g, |
| | | | 138a, 138g, 139g, 141a, 142a, 142g, 143a, 145a, 147a, 148a, 149g, 149c, 150c, 150a, 151a, 151t, 151Δ, 154g, 156c, 156a, 157a, 158a, |
| | | | 160a, 160t, 162a, 162t, 166t, 166g, 167a, 176c, 179c, 188a, 188g, 60_61ins_g, 60_61ins_c, 61_62ins_c, 81_82ins_t, 94_95ins_t, |
| | | | 98_99ins_a |
| 248 | antis_3p_NDV | 200 | 5g, 9g, 11g, 11a, 12g, 14g, 15g, 16g, 21g, 22a, 22g, 23a, 23g, 23c, 24g, 24Δ, 25g, 25Δ, 26g, 26Δ, 26c, 27g, 27Δ, 29Δ, 29g, 30Δ, 30t, 33t, |
| | | | 35t, 35a, 39g, 41g, 42g, 42c, 43c, 48a, 48t, 48c, 49a, 49c, 50a, 50t, 51g, 51t, 52a, 53t, 55t, 55c, 57t, 58a, 58g, 58c, 59c, 59a, 62c, 63a, 63c, |
| | | | 63Δ, 64a, 64Δ, 65t, 65g, 67t, 67a, 68a, 68c, 69t, 71g, 71a, 72t, 72c, 73c, 75g, 75a, 76t, 77c, 79t, 79c, 80c, 80a, 81t, 81g, 81c, 82g, 82c, 82t, |
| | | | 83c, 85t, 85g, 86g, 86t, 87c, 87t, 88t, 88c, 88a, 88g, 90t, 90g, 91t, 91c, 92a, 92t, 92c, 93t, 93c, 93g, 93Δ, 94c, 95g, 99a, 101g, 102g, 102Δ, |
| | | | 102a, 103a, 103c, 104g, 104a, 104t, 105g, 106g, 106a, 107g, 108a, 108t, 110g, 111a, 111t, 112a, 112g, 114t, 114a, 115a, 116a, 116g, 117a, |
| | | | 119g, 121g, 122a, 122g, 123g, 123a, 125t, 126a, 126g, 127a, 128a, 128g, 129a, 130g, 130a, 131g, 131a, 132g, 132a, 133g, 133a, 133c, |
| | | | 134g, 134a, 136a, 136c, 137a, 137g, 138a, 138g, 139g, 141a, 142a, 142g, 143a, 145a, 147a, 148a, 149g, 149c, 150c, 150a, 151a, 151t, |
| | | | 151Δ, 154g, 156c, 156a, 157a, 158a, 160a, 160t, 162a, 162t, 166t, 166g, 167a, 176c, 179c, 188a, 188g, 81_82ins_t, 98_99ins_a |
| 249 | antis_5p_EEEV | 163 | 5a, 6t, 9c, 10c, 10g, 14a, 19t, 21a, 23g, 23a, 27a, 28a, 30a, 30t, 33g, 33c, 34a, 34g, 35g, 36a, 37a, 37g, 38t, 39a, 42g, 51a, 51g, 54a, 57g, |
| | | | 58g, 61g, 62a, 64g, 66g, 68g, 69t, 69a, 70a, 70g, 83a, 87g, 87Δ, 88Δ, 89g, 89Δ, 90g, 90Δ, 91g, 91Δ, 92a, 92Δ, 93Δ, 94Δ, 95c, 95Δ, 96a, |
| | | | 96Δ, 97c, 97Δ, 98g, 98Δ, 99a, 99Δ, 100g, 100t, 100Δ, 101g, 101Δ, 102g, 102Δ, 103g, 103t, 103Δ, 103c, 104Δ, 105Δ, 106g, 106Δ, 107Δ, |
| | | | 108a, 108Δ, 109a, 111a, 112c, 112g, 114a, 115g, 135t, 139g, 141g, 143g, 144g, 144Δ, 146g, 146c, 147a, 149a, 152g, 154g, 162a, 162t, |
| | | | 176g, 179t, 180a, 180g, 181g, 183g, 188a, 188g, 193g, 199t, 199g, 199c, 200a, 200g, 212g, 217g, 220c, 220t, 221c, 222g, 222c, 223Δ, |
| | | | 223g, 224c, 225g, 226t, 226Δ, 227a, 227Δ, 231g, 232t, 232g, 235a, 237a, 237c, 242a, 242g, 243g, 244a, 257g, 257t, 259g, 259t, 267c, |
| | | | 268a, 277c, 278a, 280a, 282g, 283c, 285g, 287a, 288g, 289a, 290g, 14_15ins_t, 14_15ins_c, 176_177ins_g, 217_218ins_k, 217_218ins_y, |
| | | | 217_218ins_c, 217_218ins_g |
| 250 | antis_3p_EEEV | 179 | 4c, 4Δ, 4a, 5t, 5a, 5c, 6g, 6a, 7a, 7Δ, 8g, 8c, 8a, 9c, 9g, 9a, 10g, 10t, 11g, 11a, 11c, 11t, 12g, 12a, 12t, 12c, 13g, 13t, 14g, 14t, 14c, 15c, 15g, |
| | | | 17a, 17t, 17c, 18a, 18g, 18t, 18c, 20g, 20t, 21a, 21t, 21c, 22g, 22t, 24g, 24c, 24t, 25g, 27g, 28g, 29g, 29t, 31t, 36c, 38g, 38a, 38t, 39a, 40a, |
| | | | 45a, 48g, 50g, 54g, 56a, 56g, 56t, 57a, 57t, 58t, 59t, 60c, 60a, 60t, 62t, 63g, 63c, 63t, 65t, 66t, 66g, 66c, 72c, 73a, 74c, 74t, 75c, 77t, 77g, |
| | | | 81Δ, 82a, 83a, 83c, 84t, 86c, 86Δ, 87c, 87a, 87t, 91a, 92c, 93g, 93t, 95a, 95c, 96c, 96a, 100c, 100t, 102t, 102a, 105a, 105t, 108a, 108c, 111a, |
| | | | 113a, 114c, 114a, 117a, 118a, 123c, 125t, 126g, 128g, 128t, 129g, 129t, 131t, 131g, 132g, 135c, 152a, 154a, 154g, 154c, 155Δ, 156g, 156Δ, |
| | | | 157c, 157Δ, 157a, 158a, 158Δ, 158c, 158t, 159Δ, 159a, 159c, 159g, 160c, 160Δ, 161g, 161a, 161Δ, 162g, 167a, 167c, 169a, 171c, 171g, |
| | | | 172c, 178a, 178g, 180a, 183t, 183a, 184a, 3_4ins_a, 3_4ins_g, 4_5ins_g, 4_5ins_t, 84_85ins_c, 90_91ins_c, 146_147ins_g, 150_151ins_a, |
| | | | 152_153ins_a |
| 251 | antis_3p_EEEV_V2 | 196 | 4a, 4c, 5c, 6a, 7a, 7t, 7c, 7g, 8c, 8a, 9a, 9c, 11a, 12a, 12t, 13g, 13t, 14g, 15g, 16a, 16t, 18g, 18t, 19t, 19a, 20t, 21a, 21t, 22g, 24t, 27g, 29a, |
| | | | 29t, 34a, 34t, 35c, 35t, 40a, 40g, 43g, 45a, 46t, 48g, 48a, 53t, 54t, 54g, 55c, 55a, 55g, 56a, 56g, 57a, 57t, 58g, 58t, 60c, 60a, 61g, 63g, 63c, |
| | | | 66g, 66c, 70c, 72c, 73c, 74c, 74t, 75c, 77g, 81Δ, 82c, 83t, 83a, 83c, 84t, 86c, 86Δ, 87c, 87t, 88t, 90a, 91c, 91a, 92c, 95a, 95c, 96c, 96a, 100a, |
| | | | 100c, 100t, 101t, 102t, 102a, 103a, 103c, 103g, 105a, 105t, 108a, 108c, 109c, 109a, 111a, 112a, 113a, 114c, 114a, 117a, 118c, 118a, 123c, |
| | | | 124g, 124t, 125t, 126t, 126g, 127g, 128g, 128t, 129g, 129t, 130t, 130c, 131t, 131g, 132g, 135c, 149c, 152c, 152a, 153a, 153t, 154Δ, 154a, |
| | | | 154g, 154c, 155g, 155Δ, 156g, 156Δ, 157c, 157Δ, 157a, 157g, 158a, 158c, 158Δ, 158t, 159Δ, 159a, 159c, 159g, 160a, 160c, 160Δ, 161g, |
| | | | 161a, 161Δ, 162a, 162g, 164a, 165a, 165c, 166c, 166a, 166g, 167a, 167c, 168a, 169a, 171c, 171g, 172c, 175a, 178t, 178a, 178g, 179a, |
| | | | 180a, 183t, 183a, 184a, 3_4ins_a, 3_4ins_g, 4_5ins_g, 4_5ins_a, 84_85ins_c, 85_86ins_c, 90_91ins_c, 141_142ins_g, 146_147ins_g, |
| | | | 150_151ins_a, 151_152ins_a, 152_153ins_a |
| 252 | sense_5p_EEEV | 203 | 4c, 4t, 4g, 6t, 8a, 8c, 9t, 9g, 9a, 10a, 10t, 11t, 11c, 11a, 12t, 13t, 13a, 14t, 14a, 15t, 16a, 16t, 17c, 17g, 20g, 20a, 20t, 21t, 21g, 22g, 22t, 22c, |
| | | | 24t, 25g, 26t, 26g, 26a, 28a, 28Δ, 29g, 29Δ, 30c, 31Δ, 31c, 32c, 32Δ, 32t, 33c, 34Δ, 34t, 34g, 35t, 35a, 37t, 37g, 38c, 39g, 46a, 46c, 47c, |
| | | | 47g, 51c, 51g, 52a, 52c, 52g, 53t, 53a, 55a, 55g, 56g, 56a, 57t, 57g, 58t, 58a, 58c, 58g, 59c, 59a, 60t, 61g, 61t, 61c, 62a, 64a, 64g, 66c, 66a, |
| | | | 68c, 68Δ, 69Δ, 70c, 71t, 71c, 72c, 72g, 73c, 73t, 75c, 75g, 75t, 76c, 76t, 77c, 79t, 79g, 80t, 82g, 82t, 83t, 83c, 84c, 84a, 85c, 85t, 85g, 86g, |
| | | | 87c, 87a, 88g, 88c, 88t, 88a, 91c, 92a, 92t, 97g, 97t, 98t, 101g, 103Δ, 105a, 106g, 106Δ, 107c, 107Δ, 112g, 115g, 118g, 125t, 127c, 130c, |
| | | | 133g, 133t, 133c, 135a, 139t, 139c, 148c, 148a, 151c, 165c, 165a, 166a, 166c, 167a, 169c, 169a, 172t, 172a, 175c, 178t, 181t, 182t, 184g, |
| | | | 33_34ins_t, 36_37ins_t, 46_47ins_c, 99_100ins_g, 102_103ins_g, 140_141ins_c, 142_143ins_c, 147_148ins_c, 152_153ins_c, |
| | | | 154_155ins_c, 156_157ins_c, 161_162ins_c, 163_164ins_c, 164_165ins_c, 165_166ins_c, 166_167ins_c, 166_167ins_t, 167_168ins_c, |
| | | | 169_170ins_c, 170_171ins_c, 171_172ins_c, 172_173ins_c, 173_174ins_c, 174_175ins_c, 175_176ins_c, 176_177ins_c, 177_178ins_c, |
| | | | 178_179ins_c, 179_180ins_c, 180_181ins_c, 181_182ins_c, 182_183ins_c, 183_184ins_c, 185_186ins_c, 186_187ins_c, 187_188ins_c, |
| | | | 188_189ins_c |
| 253 | sense_5p_EEEV_V2 | 195 | 3t, 3a, 4c, 4t, 4g, 5a, 6t, 6a, 6c, 8a, 8c, 9t, 9g, 9a, 9c, 10a, 10t, 11t, 11c, 11a, 12t, 13t, 13a, 14t, 14a, 15t, 16a, 16t, 17g, 20g, 20t, 21t, 21g, |
| | | | 22g, 22t, 22c, 23c, 24t, 25g, 26t, 26g, 26a, 27c, 28Δ, 29g, 29Δ, 30c, 31Δ, 31c, 32c, 32Δ, 32t, 33c, 34Δ, 34t, 34g, 35t, 35a, 37t, 37g, 38c, 39g, |
| | | | 39t, 44t, 46c, 47g, 47t, 49c, 50a, 51c, 51g, 52a, 52c, 54a, 54g, 55a, 56a, 58t, 58a, 58c, 58g, 61t, 61c, 61g, 62a, 63g, 64a, 64g, 66c, 72g, 73t, |
| | | | 75g, 75t, 76t, 79g, 79t, 82g, 82t, 83t, 84a, 85t, 85g, 85c, 87a, 88t, 88a, 95g, 96t, 97g, 97t, 98t, 99g, 101g, 103Δ, 105a, 106g, 106Δ, 107c, |
| | | | 107Δ, 109t, 110g, 112g, 115g, 118g, 123t, 125t, 127c, 130c, 133g, 133t, 133c, 135a, 139t, 139c, 140c, 145c, 146t, 148c, 148a, 151c, 160t, |
| | | | 165c, 165a, 166a, 166c, 167a, 169c, 169a, 172t, 172a, 173c, 175c, 178t, 181t, 182t, 184g, 30_31ins_t, 33_34ins_t, 36_37ins_t, 46_47ins_c, |
| | | | 58_59ins_t, 99_100ins_g, 102_103ins_g, 140_141ins_c, 142_143ins_c, 147_148ins_c, 152_153ins_c, 154_155ins_c, 156_157ins_c, |
| | | | 161_162ins_c, 163_164ins_c, 164_165ins_c, 165_166ins_c, 166_167ins_c, 166_167ins_t, 167_168ins_c, 169_170ins_c, 170_171ins_c, |
| | | | 171_172ins_c, 172_173ins_c, 173_174ins_c, 174_175ins_c, 175_176ins_c, 176_177ins_c, 177_178ins_c, 178_179ins_c, 179_180ins_c, |
| | | | 180_181ins_c, 181_182ins_c, 182_183ins_c, 183_184ins_c, 185_186ins_c, 186_187ins_c, 187_188ins_c, 188_189ins_c |
| 254 | sense_3p_EEEV | 138 | 3c, 4t, 5c, 8c, 10g, 11c, 11g, 13t, 15t, 16g, 25t, 25g, 26g, 34c, 34a, 36c, 36a, 49t, 51t, 51c, 56t, 56g, 61a, 61c, 62c, 66t, 66Δ, 67a, 67Δ, 68c, |
| | | | 69g, 70Δ, 70c, 71c, 72g, 73g, 73a, 76c, 79c, 94a, 94c, 94g, 100c, 103c, 110c, 114a, 124c, 124t, 130g, 131t, 131a, 139c, 144t, 146t, 147c, |
| | | | 149c, 149Δ, 150c, 152c, 158a, 179t, 179g, 182t, 185Δ, 186Δ, 187c, 187Δ, 188Δ, 189Δ, 190c, 190a, 190Δ, 191c, 191Δ, 192c, 192Δ, 193c, |
| | | | 193a, 193Δ, 194t, 194Δ, 195c, 195Δ, 196g, 196Δ, 197t, 197Δ, 198g, 198Δ, 199Δ, 200Δ, 201t, 201Δ, 202c, 202Δ, 203c, 203Δ, 204c, 204Δ, |
| | | | 205Δ, 205t, 206c, 206Δ, 210t, 223t, 224a, 224t, 225c, 229c, 231t, 235c, 236c, 239t, 242t, 251c, 254t, 255a, 257t, 258c, 259t, 260c, 260g, |
| | | | 263t, 266t, 272t, 274a, 279t, 283g, 283c, 284g, 287a, 75_76ins_g, 75_76ins_r, 75_76ins_m, 114_115ins_c, 194_195ins_t, 276_277ins_g, |
| | | | 276_277ins_a |
| 255 | antis_5p_VEEV | 7 | 6a, 35a, 38g, 53c, 53a, 59a, 61g |
| 256 | antis_3p_VEEV | 216 | 4t, 5a, 6t, 6c, 7g, 9a, 11c, 11t, 15t, 18a, 18g, 19c, 21a, 22a, 22c, 24g, 24a, 26g, 27g, 28g, 39g, 40g, 40t, 41g, 42t, 42c, 42g, 43g, 43t, 45t, |
| | | | 45g, 45c, 46g, 46c, 46t, 53t, 53g, 54g, 55a, 55t, 58a, 58t, 58c, 61g, 63t, 65t, 69c, 70a, 70t, 71g, 71c, 73t, 74c, 74a, 75a, 75t, 75g, 76t, 76a, |
| | | | 77c, 78c, 78a, 80c, 81c, 81a, 82c, 82a, 82t, 83g, 83t, 84g, 84t, 85t, 87t, 88t, 90t, 90g, 91t, 91c, 92a, 92g, 93g, 93t, 94t, 95g, 95Δ, 96c, 96a, |
| | | | 97c, 98a, 98c, 99g, 99c, 102t, 102g, 102c, 102a, 103c, 104c, 104a, 105a, 105g, 105c, 106c, 106a, 106g, 108a, 109a, 111c, 111t, 111a, 112a, |
| | | | 112g, 113g, 113a, 114g, 114c, 115t, 115g, 116a, 118t, 120a, 121c, 122c, 122t, 123a, 124a, 124c, 124t, 125c, 126g, 127a, 128g, 131c, 134t, |
| | | | 134c, 135a, 135c, 136t, 137c, 138c, 143t, 144g, 144a, 145g, 145a, 146c, 147a, 149c, 150t, 151c, 152t, 152g, 153c, 153Δ, 153t, 154t, 154c, |
| | | | 155Δ, 155g, 155t, 155c, 156a, 156t, 156Δ, 157g, 157Δ, 157a, 158a, 158g, 158t, 159t, 159Δ, 159g, 159a, 160a, 160c, 160Δ, 160t, 161t, |
| | | | 161Δ, 161a, 162a, 162t, 162Δ, 163t, 163g, 163Δ, 163a, 166c, 166a, 167a, 169a, 170a, 170g, 171c, 172g, 173g, 174g, 175a, 176t, 177c, |
| | | | 177a, 179t, 180t, 180c, 186t, 92_93ins_a, 141_142ins_g, 147_148ins_t, 152_153ins_t, 155_156ins_t, 155_156ins_g, 168_169ins_a, |
| | | | 174_175ins_t, 174_175ins_a |
| 257 | antis_3p_VEEV_V2 | 184 | 4t, 4c, 5a, 6t, 6c, 7g, 7c, 9a, 10g, 15t, 16g, 18g, 19c, 19a, 22a, 24g, 24a, 27g, 28g, 35g, 37g, 39g, 40t, 40g, 41g, 42t, 42c, 42g, 43Δ, 43g, 43t, |
| | | | 45t, 45g, 45c, 46g, 46c, 46t, 47g, 53g, 54g, 55a, 55t, 57t, 58a, 58c, 59a, 61g, 63t, 64Δ, 67g, 70a, 70t, 72t, 73t, 75t, 75g, 76t, 76a, 78a, 79a, |
| | | | 81a, 82c, 82a, 82t, 83g, 83t, 84t, 85t, 87t, 88t, 90t, 90g, 91t, 91c, 92g, 93t, 94t, 95g, 95Δ, 96a, 97c, 99c, 102g, 102c, 103c, 105a, 105g, 105c, |
| | | | 106c, 106a, 106g, 108a, 109a, 111c, 111t, 111a, 112a, 112g, 113g, 113a, 114c, 115g, 118t, 120a, 123a, 124a, 124t, 127a, 130c, 132c, 133g, |
| | | | 134c, 135c, 136t, 137c, 141g, 142a, 144g, 144a, 145g, 145a, 147a, 149c, 150t, 151c, 152t, 153Δ, 153c, 153t, 154t, 154c, 155Δ, 155t, 155c, |
| | | | 155g, 156t, 156Δ, 157g, 157Δ, 157a, 158a, 158g, 159Δ, 159t, 159g, 159a, 160a, 160Δ, 160t, 161t, 161Δ, 161a, 162t, 162Δ, 162a, 163t, |
| | | | 163g, 163Δ, 163a, 164Δ, 166c, 169a, 170a, 172g, 173g, 176t, 177c, 177a, 181t, 186t, 92_93ins_a, 141_142ins_g, 143_144ins_a, |
| | | | 147_148ins_t, 152_153ins_t, 155_156ins_t, 155_156ins_g, 158_159ins_t, 159_160ins_c, 162_163ins_t, 163_164ins_t, 168_169ins_a, |
| | | | 174_175ins_t, 174_175ins_a |
| 258 | sense_5p_VEEV | 387 | 3c, 3a, 3t, 4a, 4Δ, 4g, 5a, 6a, 6g, 6Δ, 6t, 7a, 7g, 8Δ, 8t, 9t, 9a, 10t, 10Δ, 10g, 10a, 11t, 11c, 11a, 12a, 12c, 13c, 13a, 13Δ, 13t, 14c, 14t, 15g, |
| | | | 15c, 15a, 16g, 16c, 16a, 16t, 17a, 18a, 18Δ, 18t, 18g, 19a, 19Δ, 19t, 19g, 20a, 20t, 20Δ, 21a, 21t, 22a, 22Δ, 22t, 23t, 23a, 23Δ, 23g, 24t, 24Δ, |
| | | | 25t, 25c, 25a, 25Δ, 26a, 26c, 26Δ, 26g, 26t, 27g, 27a, 27c, 27t, 28a, 28t, 28Δ, 28c, 29a, 29c, 29t, 30Δ, 30c, 30a, 30t, 31t, 31c, 31Δ, 31a, 32c, |
| | | | 32g, 32Δ, 32a, 33a, 33c, 33g, 33Δ, 33t, 34g, 34a, 35a, 35Δ, 35t, 36g, 36c, 37t, 37a, 38g, 38t, 38c, 39g, 39t, 40c, 40t, 40a, 41c, 41t, 42g, 42a, |
| | | | 42c, 43t, 43a, 44t, 44c, 45t, 47c, 47g, 48g, 48t, 48c, 49g, 49t, 50g, 50c, 51t, 51a, 51c, 52g, 52t, 53g, 53a, 54g, 55t, 55c, 56t, 56g, 57a, 57t, |
| | | | 57c, 58t, 58Δ, 60a, 60c, 60g, 60t, 60Δ, 61c, 61g, 62a, 62g, 63a, 63c, 64t, 64g, 64c, 64a, 65a, 65g, 65t, 65c, 66a, 67t, 67a, 68a, 69t, 69a, 69g, |
| | | | 70a, 70t, 71g, 71t, 71a, 72c, 72a, 72t, 73c, 73a, 73t, 74t, 74g, 75c, 75a, 75t, 76t, 76c, 76a, 77t, 77c, 78a, 78c, 79t, 79c, 79a, 80a, 80t, 80c, |
| | | | 81a, 81c, 82c, 82a, 83g, 83t, 83c, 85a, 85t, 87a, 87g, 88c, 88g, 88a, 88t, 89t, 89a, 90a, 90c, 91g, 91a, 92g, 92a, 92c, 93a, 93c, 94c, 94a, 95t, |
| | | | 95a, 96a, 96c, 97a, 98a, 98t, 99a, 99c, 100a, 100c, 101a, 102c, 103c, 103t, 103g, 105g, 105t, 106c, 106g, 106t, 107g, 107t, 107a, 108g, 108t, |
| | | | 108c, 109g, 109a, 110a, 110t, 110c, 111a, 111g, 112t, 113g, 113t, 113c, 113a, 114g, 114c, 115c, 115g, 115a, 116g, 116c, 117g, 117c, 120a, |
| | | | 121a, 121t, 121c, 121g, 123g, 123c, 125t, 125c, 127a, 127c, 129c, 129t, 130c, 131t, 132c, 132a, 133c, 133a, 133g, 133t, 134g, 135c, 135a, |
| | | | 135g, 136c, 136g, 137c, 137a, 138t, 138c, 139c, 139a, 141c, 141a, 142a, 142c, 143c, 143g, 143a, 146c, 147c, 147a, 147g, 148t, 148a, 148c, |
| | | | 149c, 149a, 151c, 151a, 152c, 152t, 153c, 153a, 154c, 154t, 155c, 156t, 156c, 157c, 157a, 158t, 159a, 159t, 160c, 160t, 161c, 161t, 163g, |
| | | | 163a, 164t, 165t, 165a, 169t, 170t, 170a, 170c, 172t, 173c, 173t, 174t, 174a, 175a, 176g, 176t, 177a, 177t, 179c, 179t, 180t, 182a, 185a, |
| | | | 11_12ins_c, 13_14ins_t, 13_14ins_a, 19_20ins_t, 29_30ins_g, 29_30ins_a, 32_33ins_c, 32_33ins_a, 33_34ins_a, 41_42ins_a, 60_61ins_t |
| 259 | sense_5p_VEEV_V2 | 353 | 3c, 3a, 3t, 4Δ, 4a, 4g, 5Δ, 5a, 5g, 6g, 6a, 6Δ, 6t, 6c, 7Δ, 7a, 7t, 8a, 8Δ, 8t, 9t, 9a, 10a, 10Δ, 10t, 10g, 11t, 11c, 11a, 12a, 12c, 13a, 13c, 13t, |
| | | | 13Δ, 14c, 14t, 15g, 15a, 16c, 16a, 16t, 17a, 18a, 18Δ, 18t, 18g, 19a, 19Δ, 19t, 19g, 20a, 20Δ, 21a, 21t, 22a, 22Δ, 22t, 23t, 23a, 23Δ, 23g, 24t, |
| | | | 24Δ, 25c, 25a, 25Δ, 25t, 26a, 26c, 26Δ, 26g, 26t, 27a, 27c, 27t, 28a, 28t, 28Δ, 28c, 29a, 29c, 29t, 30Δ, 30a, 30t, 31t, 31c, 31Δ, 31a, 32c, 32g, |
| | | | 32Δ, 32a, 33a, 33g, 33Δ, 33c, 33t, 34a, 34g, 35a, 35Δ, 35t, 36c, 37t, 37a, 38g, 38t, 38c, 39t, 39g, 40c, 40t, 40a, 41c, 41t, 42g, 42c, 43t, 43a, |
| | | | 44t, 44c, 45t, 47c, 47g, 48t, 48c, 49t, 50g, 50c, 51t, 51c, 51g, 52g, 52t, 53g, 54g, 55t, 55c, 56t, 56g, 57a, 57t, 57c, 58t, 58Δ, 59a, 60a, 60t, |
| | | | 60Δ, 61c, 62a, 62g, 63a, 63c, 64c, 64a, 65a, 65t, 65c, 66a, 67t, 67a, 68a, 69a, 69g, 70a, 70t, 71g, 71t, 71a, 72a, 72t, 73a, 73c, 73t, 74t, 74g, |
| | | | 75c, 75a, 75t, 76t, 76c, 76a, 77t, 77c, 78a, 78c, 79t, 79c, 79a, 80a, 80t, 80c, 81a, 81c, 82c, 82a, 83g, 83t, 83c, 84g, 85a, 85t, 87a, 88g, 88a, |
| | | | 89a, 90a, 91a, 92g, 92a, 92c, 93a, 94a, 95a, 96a, 96c, 97a, 98a, 98t, 99a, 100a, 100c, 101a, 102c, 103t, 104t, 105t, 106c, 106t, 107g, 107t, |
| | | | 107a, 108t, 108c, 109a, 110a, 110t, 110c, 111a, 111g, 113c, 113a, 114c, 115g, 115a, 116c, 117g, 117c, 118c, 121t, 121a, 121c, 121g, 123g, |
| | | | 123c, 124g, 125t, 125c, 127c, 129c, 129t, 130c, 131t, 132c, 133c, 133a, 133g, 133t, 134g, 135a, 135g, 135c, 136g, 137c, 137a, 138c, 139c, |
| | | | 139a, 140a, 141c, 141a, 142a, 142c, 143c, 143g, 143a, 144t, 146c, 147c, 147a, 147g, 148t, 148a, 148c, 149c, 149a, 151c, 151a, 152c, 152t, |
| | | | 153c, 153a, 154c, 154t, 155c, 156c, 157c, 157a, 158t, 159a, 159t, 160c, 160t, 161c, 161t, 163a, 164t, 165a, 166a, 170t, 170a, 170c, 172t, |
| | | | 173c, 173t, 174t, 175a, 176t, 177a, 177t, 178a, 179c, 179t, 180t, 182a, 185a, 11_12ins_c, 13_14ins_t, 13_14ins_a, 19_20ins_t, 24_25ins_a, |
| | | | 29_30ins_g, 29_30ins_a, 32_33ins_c, 32_33ins_a, 33_34ins_a, 41_42ins_a, 44_45ins_t, 60_61ins_t, 62_63ins_t |
| 260 | sense_3p_VEEV | 6 | 3g, 4c, 6t, 27c, 30t, 59t |
| 261 | antis_5p_WEEV | 71 | 4a, 4Δ, 5c, 6t, 10c, 10t, 11g, 11t, 13c, 13g, 15c, 17a, 19a, 21t, 21a, 24t, 24c, 25c, 25a, 34a, 34t, 34c, 37g, 41a, 41t, 41g, 43g, 50t, 55g, 57g, |
| | | | 58g, 58t, 62a, 63t, 68g, 69a, 70a, 74g, 77t, 77g, 87g, 87t, 90t, 92a, 101a, 112g, 115g, 120t, 120g, 121g, 122g, 128a, 128g, 136c, 137g, 138t, |
| | | | 149c, 149t, 151a, 152g, 156g, 202a, 209t, 214a, 214Δ, 26_27ins_g, 26_27ins_a, 112_113ins_c, 123_124ins_g, 193_194ins_t, |
| | | | 193_194ins_a |
| 262 | antis_3p_WEEV | 17 | 6a, 7a, 7Δ, 20t, 41a, 62c, 62t, 65c, 67c, 80c, 81g, 116g, 155t, 155g, 158c, 171g, 182a |
| 263 | antis_3p_WEEV_V2 | 31 | 7a, 7c, 7g, 11t, 23g, 33c, 38g, 40a, 41a, 54g, 55a, 57t, 62t, 78g, 79c, 80c, 80g, 81g, 89a, 115g, 116g, 154t, 154g, 155t, 155g, 157c, 158c, |
| | | | 170g, 171g, 181a, 182a |
| 264 | sense_5p_WEEV | 16 | 9t, 11g, 20c, 27c, 33g, 36a, 36c, 50c, 52c, 56a, 70t, 70c, 75c, 86c, 87g, 87a |
| 265 | sense_5p_WEEV_V2 | 34 | 3t, 3a, 6a, 6c, 9t, 9c, 11g, 20c, 27c, 31t, 33g, 34g, 36a, 36c, 48c, 50c, 51c, 51g, 52c, 58g, 70c, 75c, 84t, 86c, 87g, 87a, 98t, 102t, 117t, 120g, |
| | | | 126t, 153c, 174a, 184t |
| 266 | sense_3p_WEEV | 92 | 3a, 3c, 4g, 4Δ, 6t, 9a, 9c, 10c, 11a, 11t, 16c, 18t, 19g, 19c, 20t, 22a, 23a, 33t, 39Δ, 39t, 41a, 42t, 49a, 54c, 55c, 61c, 61t, 64c, 68a, 69t, 69c, |
| | | | 71g, 71c, 72c, 73c, 74c, 76c, 81c, 83c, 84g, 88c, 90t, 96t, 98c, 100a, 100c, 104g, 104c, 107g, 112a, 120a, 122a, 126g, 133c, 133a, 138g, |
| | | | 140c, 140t, 142t, 143a, 143c, 144t, 146c, 147g, 147t, 148g, 149t, 151t, 151a, 158t, 160a, 162c, 162g, 165c, 183c, 186t, 186a, 195g, 199t, |
| | | | 201t, 205g, 207c, 207a, 209c, 214a, 7_8ins_c, 26_27ins_a, 26_27ins_t, 191_192ins_c, 191_192ins_t, 193_194ins_c, 193_194ins_t |

What is claimed is:

1. An isolated RNA polynucleotide, comprising
a coding region having a coding sequence encoding one or more therapeutic polypeptides; and
template regions, wherein the template regions comprise two distinct regions, a left flanking region (“L region”) of a virus and a right flanking region (“R region”) of the virus, wherein the L region is adjacent to and contiguous with a 5′ end of the coding region and the R region is adjacent to and contiguous with a 3′ end of the coding region;
wherein the coding sequence is in an antisense orientation;
wherein the therapeutic polypeptide is heterologous to the virus; and
wherein the template regions interact with and initiate RNA-dependent polymerase activity of a polymerase in a cell containing the RNA dependent polymerase.

2. The reverse complement of the isolated RNA polynucleotide of claim 1.

3. The isolated RNA polynucleotide of claim 1 or 2, wherein the virus is selected from the group consisting of viruses in the orders of Amarillovirales, Articulavirales, Blubervirales, Bunyavirales, Hepelivirales, Martellivirales, Mononegavirales, Nidovirales, and Picornavirales.

4. The isolated RNA polynucleotide of any one of claims 1-3, wherein the virus is selected from the group consisting of viruses in the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, Rhabdoviridae, and Togaviridae.

5. The isolated RNA polynucleotide of any one of claims 1-4, wherein the virus is selected from the group consisting of Alphacoronavirus 229E, Alphacoronavirus NL63, Alphacoronavirus WA2028, Avian metapneumovirus (AMPV), Betacoronavirus HKU1, Betacoronavirus HKU15, Betacoronavirus HKU33, Betacoronavirus OC43, Chikungunya virus, Crimean-Congo Hemorrhagic Fever Virus, Dengue Virus, Eastern Equine Encephalitis Virus (EEEV), Enterovirus D68 (EV-D68), Foot and Mouth Disease Virus, Hanta Virus, Hendra Virus, Hepatitis B Virus, Hepatitis C Virus, HMPV, Human Parainfluenzavirus 1 (HPIV1), Human Parainfluenzavirus 3 (HPIV3), Infectious Salmon Anemia Virus, Influenza A Virus, Influenza B Virus, Lassa Virus, Marburg Virus, Middle East Respiratory Syndrome Coronavirus (MERS-COV), Newcastle Disease Virus (NDV), Nipah Virus, Norwalk Virus, Rabies Virus, Respiratory Syncytial Virus, Reston Ebola virus, Rhinovirus, Rift Valley Fever Virus, Rubella virus, SARS-COV-1, SARS-COV-2, Sudan Ebola virus, Venezuelan Equine Encephalitis Virus (VEEV), Vesicular Stomatitis Virus, Western Equine Encephalitis Virus (WEEV), Yellow Fever Virus, Zaire Ebola virus, and Zika Virus.

6. The isolated RNA polynucleotide of any one of claims 1-5, wherein the virus is not an alphavirus.

7. The isolated RNA polynucleotide of any one of claims 1-6, wherein the template regions are native to the virus.

8. The isolated RNA polynucleotide of any one of claims 1-7, wherein the template regions are variants of template regions native to the virus, wherein the variants have at least 90%, 91%, 92%, 93%, 94%, 95%, 96%, 97%, 98%, or 99% sequence identity to the template regions native to the virus.

9. The isolated RNA polynucleotide of any one of claims 1-8, wherein each of the L and the R regions of the template regions comprise fewer than 10, 9, 8, 7, 6, 5, 4, 3, or 2 variations relative to template regions native to the virus.

10. The isolated RNA polynucleotide of any one of claims 1-9, wherein each of the L and the R regions of the template regions vary from template regions native to the virus by not more than 10, 9, 8, 7, 6, 5, 4, 3, or 2 substitutions that are not involved in 5′ capping.

11. The isolated RNA polynucleotide of any one of claims 1-10, wherein each of the L and the R regions of the template regions vary from template regions native to the virus by not more than 1 substitution that is not involved in 5′ capping.

12. The isolated RNA polynucleotide of any one of claims 1-11, wherein the isolated RNA polynucleotide comprises at least one nucleoside modification.

13. The isolated RNA polynucleotide of any one of claims 1-12, wherein the template regions are nucleoside modified, wherein the percentage of modified nucleosides is not more than 40%, 35%, 30%, 25%, 20%, 15%, 10%, or 5%.

14. The isolated RNA polynucleotide of any one of claims 1-12, wherein the template regions are nucleoside modified, wherein the percentage of modified nucleosides at least 30%, 35%, 40%, 45%, 50%, 55%, 60%, 65%, 70%, 75%, 80%, 90%, 95%, or 100%.

15. The isolated RNA polynucleotide of claim 12 or 13, wherein the nucleoside modification is a nonimmunogenic uridine modification, and the percentage of modified uridine modifications is not more than 40%, 35%, 30%, 25%, 20% 15% or 10%.

16. The isolated RNA polynucleotide of any one of claim 12 or 14, wherein the nucleoside modification is a nonimmunogenic uridine modification, and the percentage of modified uridine modifications is more than 30%, 35%, 40%, 45%, 50%, 55%, 60%, 65%, 70%, 75%, 80%, 90%, or 95%, or is 100%.

17. The isolated RNA polynucleotide of claim 12 or 13, wherein the nucleoside modification is a nonimmunogenic cytidine modification, and the percentage of modified cytidine modifications is not more than 40%, 35%, 30%, 25%, 20% 15% or 10%.

18. The isolated RNA polynucleotide of any one of claim 12 or 14, wherein the nucleoside modification is a nonimmunogenic cytidine modification, and the percentage of modified cytidine modifications is more than 30%, 35%, 40%, 45%, 50%, 55%, 60%, 65%, 70%, 75%, 80%, 90%, or 95%, or is 100%.

19. The isolated RNA polynucleotide of claim 12 or 13, wherein the nucleoside modification is a nonimmunogenic adenosine modification, and the percentage of modified adenosine modifications is between 1% and 30%.

20. The isolated RNA polynucleotide of claim 19, wherein the nucleoside modification is a nonimmunogenic adenosine modification, and the percentage of modified cytidine modifications is about 1%, 5%, 10%, 15%, 20%, 25%, or 30%.

21. The isolated RNA polynucleotide of any one of claims 1-20, wherein the isolated polynucleotide comprises a 5′ cap structure.

22. The isolated RNA polynucleotide of any one of claims 1-21, wherein the 5′ end of the L region comprises a 5′ cap structure.

23. The isolated RNA polynucleotide of any one of claims 1-22, wherein the 5′ end of the L region comprises one or more variations associated with a 5′ cap structure.

24. The isolated RNA polynucleotide of claim 22 or 23, wherein the 5′ cap structure is selected from the group consisting of Cap 0, Cap 0 (3′-O-Me), Cap 1, Cap 1 (3′-O-Me), Cap 2, Cap 2 (3′-O-Me), Anti-Reverse Cap Analog (ARCA), inosine, N1-methyl-guanosine, 2′-fluoro-guanosine, 7-deaza-guanosine, 8-oxo-guanosine, 2-amino-guanosine, locked nucleic acid guanosine (LNA-guanosine), and 2-azido-guanosine structure.

25. The isolated RNA polynucleotide of any one of claims 1-20, wherein the isolated polynucleotide does not comprise a 5′ cap structure (uncapped).

26. The isolated RNA polynucleotide of any one of claim 1-20 or 25, wherein the 5′ end of the L region does not comprise a 5′ cap structure (uncapped).

27. The isolated RNA polynucleotide of claim 25 or 26, wherein the 5′ end of the isolated polynucleotide comprises a 5′-monophosphate, 5′-diphosphate, or 5′-triphosphate.

28. The isolated RNA polynucleotide of claim 25 or 26, wherein the 5′ end of the isolated polynucleotide does not comprise a 5′-phosphate (dephosphorylated).

29. The isolated RNA polynucleotide of claim 2, wherein the template regions are the reverse complement of template regions native to the virus.

30. The isolated RNA polynucleotide of claim 29, wherein the template regions are variants of a reverse complement of a template regions native to the virus, wherein the variants have at least 90%, 91%, 92%, 93%, 94%, 95%, 96%, 97%, 98%, or 99% sequence identity to the reverse complement of the template regions native to the virus.

31. The isolated RNA polynucleotide of claim 29 or 30, wherein the reverse complements of each of the L and the R regions vary from the reverse complements of template regions native to the virus by not more than 10, 9, 8, 7, 6, 5, 4, 3, or 2 substitutions that are not involved in 5′ capping.

32. The isolated RNA polynucleotide of any one of claims 29-31, wherein the reverse complements of each of the L and the R regions vary from the reverse complements of a template region native to the virus by not more than 1 substitution that is not involved in 5′ capping.

33. The isolated RNA polynucleotide of any one of claims 29-32, wherein the isolated RNA polynucleotide comprises at least one nucleoside modification.

34. The isolated RNA polynucleotide of claim 33, wherein the template regions are nucleoside-modified and the percentage of modified nucleotides is not more than 40%, 35%, 30%, 25%, 20%, 15%, 10%, or 5%.

35. The isolated RNA polynucleotide of any one of claim 2 or 29-34, wherein the 5′ end of the reverse complement of the R region encodes a cap structure.

36. The isolated RNA polynucleotide of any one of claim 2 or 29-35, wherein the 5′ end of the R region is capped.

37. The isolated RNA polynucleotide of any one of claim 1-36, wherein the therapeutic polypeptide is a secreted polypeptide.

38. The isolated RNA polynucleotide of any one of claims 1-37, wherein the therapeutic polypeptide is selected from the group consisting of an interferon, an interferon stimulated gene, a cytokine, a chemokine, an antibody, a signaling molecule, a cytotoxic protein, a protein that causes cell death, an antineoplastic protein, an immunomodulatory protein, protein toll-like receptor agonist, or a dominant negative protein.

39. The isolated RNA polynucleotide of claim 38, wherein the cytokine is an inflammatory cytokine.

40. The isolated RNA polynucleotide of claim 38, wherein the inflammatory cytokine is TNF-α.

41. The isolated RNA polynucleotide of claim 38, wherein the cytokine is an anti-inflammatory cytokine.

42. The isolated RNA polynucleotide of claim 41, wherein the anti-inflammatory cytokine is an interleukin-1 receptor antagonist (IL-1RN).

43. The isolated RNA polynucleotide of any one of claim 1-42, wherein the therapeutic polypeptide is an interleukin or a caspase.

44. The isolated RNA polynucleotide of claim 43, wherein the interleukin is IL-12A, IL-12B or IL-2.

45. The isolated RNA polynucleotide of claim 38, wherein the secreted protein is an antibody.

46. The isolated RNA polynucleotide of claim 38, wherein the therapeutic polypeptide is an interferon.

47. The isolated RNA polynucleotide of claim 46, wherein the interferon is an IFN-α, IFN-β, IFN-ε, IFN-κ, IFN-ω, IFN-γ, or IFN-λ.

48. The isolated RNA polynucleotide of claim 47, wherein the interferon is IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-β1, IFN-ε, IFN-κ, IFN-ω1, IFN-γ, IFN-λ1 (IL28A), IFN-λ2 (IL28B), IFN-λ3 (IL29), or IFN-λ4.

49. The isolated RNA polynucleotide of claim 46, wherein the interferon is IFN-α, IFN-β, IFN-κ, IFN-λ1 (IL28A), IFN-λ2 (IL28B), or IFN-λ3 (IL29).

50. The isolated RNA polynucleotide of any one of claims 1-49, wherein the coding sequence encodes more than one therapeutic polypeptide, which are separated by one or more ribosomal skipping sequence.

51. The isolated RNA polynucleotide of any one of claims 1-50, wherein the coding region further comprises one or more regulatory elements selected from the group consisting of ribosomal binding site, Kozak sequence, Shine-Dalgarno sequence, ribozyme, riboswitch, promoter, microRNA binding site, and internal ribosomal entry site (IRES).

52. The isolated RNA polynucleotide of any one of claims 1-51, wherein the one or more regulatory elements are operably linked to the coding sequence.

53. The isolated RNA polynucleotide of any one of claims 1-52, further comprising a polyadenylation signal and/or a 3′ poly(A) tail.

54. The isolated RNA polynucleotide of any one of claim 1-53, wherein the RNA-dependent polymerase is an RNA-dependent RNA polymerase.

55. The isolated RNA polynucleotide of any one of claims 1-53, wherein the RNA-dependent polymerase is an RNA-dependent DNA polymerase.

56. The isolated RNA polynucleotide of any one of claims 1-55, wherein the RNA-dependent polymerase is a polymerase is from the virus.

57. The isolated RNA polynucleotide of any one of claims 1-56, wherein the isolated RNA polynucleotide is a single stranded RNA.

58. The isolated RNA polynucleotide of any one of claims 1-57, wherein the isolated polynucleotide is in linear form.

59. The isolated RNA polynucleotide of any one of claims 1-57, wherein the isolated polynucleotide is in a covalently-closed circular form.

60. The isolated RNA polynucleotide of any one of claims 1-59, wherein
the virus is an influenza virus,
wherein the L region comprises a nucleotide sequence set forth as SEQ ID NO: 2; or a variant of SEQ ID NO: 2, wherein the variant of SEQ ID NO: 2 comprises a variation at one or more nucleotide positions selected from the group consisting of positions 14-26 of SEQ ID NO: 2; and
wherein the R region comprises the nucleotide sequence set forth as any one of SEQ ID NOs: 20, 21, 22, or 23; or a variant of any one of SEQ ID NOs: 20, 21, 22, or 23, wherein the variant comprises a variation at one or more nucleotide positions selected from the group consisting of positions 8-15 of any one of SEQ ID NOs: 20, 21, 22, or 23.

61. The isolated RNA polynucleotide of any one of claims 1-59, wherein
the virus is an influenza virus,
wherein the L region comprises a nucleotide sequence set forth as SEQ ID NO: 3; or a variant of SEQ ID NO: 3, wherein
(i) the variant of SEQ ID NO: 3 comprises a variation at one or more nucleotide positions selected from the group consisting of positions 14-35 of SEQ ID NO: 3; and
wherein the R region comprises the nucleotide sequence set forth as any one of SEQ ID NOs: 24, 25, 26, or 27; or a variant of any one of SEQ ID NOs: 24, 25, 26, or 27, wherein
(i) the variant of SEQ ID NO: 24 comprises a variation at one or more nucleotide positions selected from the group consisting of positions 8-12 of SEQ ID NO: 24;
(ii) the variant of SEQ ID NO: 25 comprises a variation at one or more nucleotide positions selected from the group consisting of positions 8-12 of SEQ ID NO: 25;
(iii) the variant of SEQ ID NO: 26 comprises a variation at one or more nucleotide positions selected from the group consisting of positions 8-12 of SEQ ID NO: 26; or
(iv) the variant of SEQ ID NO: 27 comprises a variation at one or more nucleotide positions selected from the group consisting of positions 8-12 of SEQ ID NO: 27.

62. The isolated RNA polynucleotide of any one of claims 1-59, wherein
the virus is an influenza virus,
wherein the L region comprises a nucleotide sequence set forth as SEQ ID NO: 4; or a variant of SEQ ID NO: 4, wherein the variant of SEQ ID NO: 4 comprises a variation at one or more nucleotide positions selected from the group consisting of positions 14-50 of SEQ ID NO: 4; and
wherein the R region comprises the nucleotide sequence set forth as any one of SEQ ID NOs: 28, 29, 30, or 31; or a variant of any one of SEQ ID NOs: 28, 29, 30, or 31, wherein the variant comprises a variation at one or more nucleotide positions selected from the group consisting of positions 8-12 of any one of SEQ ID NOs: 28, 29, 30, or 31.

63. The isolated RNA polynucleotide of any one of claims 1-59, wherein
the virus is an influenza virus,
wherein the L region comprises a nucleotide sequence set forth as SEQ ID NO: 1 or 5; or a variant of SEQ ID NO: 1 or 5, wherein the variant comprises a variation at one or more nucleotide positions selected from the group consisting of positions 14-37 of SEQ ID NO: 1 or 5; and
wherein the R region comprises the nucleotide sequence set forth as SEQ ID NO: 18 or 19; or a variant of SEQ ID NO: 18 or 19, wherein the variant comprises a variation at one or more nucleotide positions selected from the group consisting of positions 8-20 of SEQ ID NO: 18 or 19.

64. The isolated RNA polynucleotide of any one of claims 1-59, wherein
the virus is an influenza virus,
wherein the L region comprises a nucleotide sequence set forth as SEQ ID NO: 6; or a variant of SEQ ID NO: 6, wherein the variant of SEQ ID NO: 6 comprises a variation at one or more nucleotide positions selected from position 14 or 15 of SEQ ID NO: 6; and
wherein the R region comprises the nucleotide sequence set forth as SEQ ID NO: 32 or 33; or a variant of SEQ ID NO: 32 or 33, wherein the variant comprises a variation at one or more nucleotide positions selected from the group consisting of positions 8-33 of SEQ ID NO: 32 or 33.

65. The isolated RNA polynucleotide of any one of claims 1-59, wherein
the virus is an influenza virus,
wherein the L region comprises a nucleotide sequence set forth as SEQ ID NO: 7; or a variant of SEQ ID NO: 7, wherein the variant of SEQ ID NO: 7 comprises a variation at one or more nucleotide positions selected from the group consisting of positions 14-20 of SEQ ID NO: 7; and
wherein the R region comprises the nucleotide sequence set forth as any one of SEQ ID NOs: 34, 35, 36, or 37; or a variant of any one of SEQ ID NOs: 34, 35, 36, or 37, wherein the variant comprises a variation at one or more nucleotide positions selected from the group consisting of positions 5-8 of SEQ ID NO: 34, 35, 36, or 37.

66. The isolated RNA polynucleotide of any one of claims 1-59, wherein
the virus is an influenza virus,
wherein the L region comprises a nucleotide sequence set forth as SEQ ID NO: 8; or a variant of SEQ ID NO: 8, wherein the variant of SEQ ID NO: 8 comprises a variation at one or more nucleotide positions selected from position 14 or 15 of SEQ ID NO: 8; and
wherein the R region comprises the nucleotide sequence set forth as any one of SEQ ID NOs: 38, 39, 40, or 41; or a variant of any one of SEQ ID NOs: 38, 39, 40, or 41, wherein the variant comprises a variation at one or more nucleotide positions selected from the group consisting of positions 8-13 of SEQ ID NO: 38, 39, 40, or 41.

67. The isolated RNA polynucleotide of any one of claims 1-59, wherein
the virus is an influenza virus,
wherein the L region comprises a nucleotide sequence set forth as SEQ ID NO: 9; or a variant of SEQ ID NO: 9, wherein the variant of SEQ ID NO: 9 comprises a variation at one or more nucleotide positions selected from the group consisting of positions 14-18 of SEQ ID NO: 9; and
wherein the R region comprises the nucleotide sequence set forth as SEQ ID NO: 42 or 43; or a variant of SEQ ID NO: 42 or 43, wherein the variant comprises a variation at one or more nucleotide positions selected from the group consisting of positions 8-14 of SEQ ID NO: 42 or 43.

68. The isolated RNA polynucleotide of any one of claims 1-59, wherein
the virus is an influenza virus,
wherein the L region comprises a nucleotide sequence set forth as SEQ ID NO: 11; or a variant of SEQ ID NO: 11, wherein the variant of SEQ ID NO: 11 comprises a variation at one or more nucleotide positions selected from the group consisting of positions 14-81 of SEQ ID NO: 11; and
wherein the R region comprises the nucleotide sequence set forth as SEQ ID NO: 46 or 47; or a variant of SEQ ID NO: 46 or 47, wherein the variant comprises a variation at one or more nucleotide positions selected from the group consisting of positions 5-9 of SEQ ID NO: 46 or 47.

69. The isolated RNA polynucleotide of any one of claims 1-59, wherein
the virus is an influenza virus,
wherein the L region comprises a nucleotide sequence set forth as SEQ ID NO: 12; or a variant of SEQ ID NO: 12, wherein the variant of SEQ ID NO: 12 comprises a variation at one or more nucleotide positions selected from the group consisting of positions 14-52 of SEQ ID NO: 12; and
wherein the R region comprises the nucleotide sequence set forth as any one of SEQ ID NO: 48 or 49; or a variant of any one of SEQ ID NO: 48 or 49, wherein the variant comprises a variation at one or more nucleotide positions selected from the group consisting of positions 8-11 of SEQ ID NO: 48 or 49.

70. The isolated RNA polynucleotide of any one of claims 1-59, wherein
the virus is an influenza virus,
wherein the L region comprises a nucleotide sequence set forth as SEQ ID NO: 13; or a variant of SEQ ID NO: 13, wherein the variant of SEQ ID NO: 13 comprises a variation at one or more nucleotide positions selected from the group consisting of positions 14-87 of SEQ ID NO: 13; and
wherein the R region comprises the nucleotide sequence set forth as SEQ ID NO: 50 or 51; or a variant of SEQ ID NO: 50 or 51, wherein the variant comprises a variation at one or more nucleotide positions selected from the group consisting of positions 8-17 of SEQ ID NO: 50 or 51.

71. The isolated RNA polynucleotide of any one of claims 1-59, wherein
the virus is an influenza virus,
wherein the L region comprises a nucleotide sequence set forth as any one of SEQ ID NO: 10; or a variant of any one of SEQ ID NO: 10, wherein the variant of SEQ ID NO: 10 comprises a variation at one or more nucleotide positions selected from the group consisting of positions 14-86 of SEQ ID NO: 10; and
wherein the R region comprises the nucleotide sequence set forth as SEQ ID NO: 44 or 45; or a variant of SEQ ID NO: 44 or 45, wherein the variant comprises a variation at one or more nucleotide positions selected from the group consisting of positions 8-21 of SEQ ID NO: 44 or 45.

72. The isolated RNA polynucleotide of any one of claims 1-59, wherein
the virus is an influenza virus,
wherein the L region comprises a nucleotide sequence set forth as SEQ ID NO: 14; or a variant of SEQ ID NO: 14, wherein the variant of SEQ ID NO: 14 comprises a variation at one or more nucleotide positions selected from the group consisting of positions 14-93 of SEQ ID NO: 14; and
wherein the R region comprises the nucleotide sequence set forth as any one of SEQ ID NO: 52 or 53; or a variant of any one of SEQ ID NO: 52 or 53, wherein the variant comprises a variation at one or more nucleotide positions selected from the group consisting of positions 8-48 of SEQ ID NO: 52 or 53.

73. The isolated RNA polynucleotide of any one of claims 1-59, wherein
the virus is an influenza virus,
wherein the L region comprises a nucleotide sequence set forth as SEQ ID NO: 15; or a variant of SEQ ID NO: 15, wherein the variant of SEQ ID NO: 15 comprises a variation at one or more nucleotide positions selected from the group consisting of positions 14-95 of SEQ ID NO: 15; and
wherein the R region comprises the nucleotide sequence set forth as SEQ ID NO: 54 or 55; or a variant of any one of SEQ ID NO: 54 or 55, wherein the variant comprises a variation at one or more nucleotide positions selected from the group consisting of positions 8-34 of SEQ ID NO: 54 or 55.

74. The isolated RNA polynucleotide of any one of claims 1-59, wherein
the virus is an influenza virus,
wherein the L region comprises a nucleotide sequence set forth as SEQ ID NO: 16; or a variant of SEQ ID NO: 16, wherein the variant of SEQ ID NO: 16 comprises a variation at one or more nucleotide positions selected from the group consisting of positions 14-81 of SEQ ID NO: 16; and
wherein the R region comprises the nucleotide sequence set forth as any one of SEQ ID NO: 56 or 57; or a variant of any one of SEQ ID NO: 56 or 57, wherein the variant comprises a variation at one or more nucleotide positions selected from the group consisting of positions 8-12 of SEQ ID NO: 56 or 57.

75. The isolated RNA polynucleotide of any one of claims 1-59, wherein
the virus is an influenza virus,
wherein the L region comprises a nucleotide sequence set forth as SEQ ID NO: 17; or a variant of SEQ ID NO: 17, wherein the variant of SEQ ID NO: 17 comprises a variation at one or more nucleotide positions selected from the group consisting of positions 14-22 of SEQ ID NO: 17; and
wherein the R region comprises the nucleotide sequence set forth as any one of SEQ ID NO: 58 or 59; or a variant of any one of SEQ ID NO: 58 or 59, wherein the variant comprises a variation at one or more nucleotide positions selected from the group consisting of positions 8-32 of SEQ ID NO: 58 or 59.

76. The isolated RNA polynucleotide of any one of claims 1-59, wherein
the virus is a sarbecovirus,
wherein the L region comprises a nucleotide sequence set forth as SEQ ID NO: 137; or a variant of SEQ ID NO: 137, wherein the variant of SEQ ID NO: 137 comprises a variation at one or more nucleotide positions selected from the group consisting of positions 40-1557 of SEQ ID NO: 137; and
wherein the R region comprises the nucleotide sequence set forth as SEQ ID NO: 128; or a variant of any one of SEQ ID NO: 128, wherein the variant of SEQ ID NO: 128 comprises a variation at one or more nucleotide positions selected from the group consisting of positions 20-30 of SEQ ID NO: 128.

77. The isolated RNA polynucleotide of any one of claims 1-59, wherein
the virus is a sarbecovirus,
wherein the L region comprises a nucleotide sequence set forth as any one of SEQ ID NOs: 138, 139, 140, 141, 142, 143, or 144; or a variant of any one of SEQ ID NOs: 138, 139, 140, 141, 142, 143, or 144, wherein
(i) the variant of SEQ ID NO: 138 comprises a variation at one or more nucleotide positions selected from the group consisting of positions 50-312 of SEQ ID NO: 138;
(ii) the variant of SEQ ID NO: 139 comprises a variation at one or more nucleotide positions selected from the group consisting of positions 50-1567 of SEQ ID NO: 139;
(iii) the variant of SEQ ID NO: 140 comprises a variation at one or more nucleotide positions selected from the group consisting of positions 50-1488 of SEQ ID NO: 140;
(iv) the variant of SEQ ID NO: 141 comprises a variation at one or more nucleotide positions selected from the group consisting of positions 50-1593 of SEQ ID NO: 141;
(v) the variant of SEQ ID NO: 142 comprises a variation at one or more nucleotide positions selected from the group consisting of positions 50-1570 of SEQ ID NO: 142;
(vi) the variant of SEQ ID NO: 143 comprises a variation at one or more nucleotide positions selected from the group consisting of positions 50-1488 of SEQ ID NO: 143; or
(vii) the variant of SEQ ID NO: 144 comprises a variation at one or more nucleotide positions selected from the group consisting of positions 50-1593 of SEQ ID NO: 144; and
wherein the R region comprises the nucleotide sequence set forth as any one of SEQ ID NOs: 130, 136, 145, 146, or 147; or a variant of any one of SEQ ID NOs: 130, 136, 145, 146, or 147, wherein
(i) the variant of SEQ ID NO: 130 comprises a variation at one or more nucleotide positions selected from the group consisting of positions 20-320 of SEQ ID NO: 130;
(ii) the variant of SEQ ID NO: 136 comprises a variation at one or more nucleotide positions selected from the group consisting of positions 20-33 of SEQ ID NO: 136;
(iii) the variant of SEQ ID NO: 145 comprises a variation at one or more nucleotide positions selected from the group consisting of positions 50-1461 of SEQ ID NO: 145;
(iv) the variant of SEQ ID NO: 146 comprises a variation at one or more nucleotide positions selected from the group consisting of positions 60-1441 of SEQ ID NO: 146; or
(v) the variant of SEQ ID NO: 147 comprises a variation at one or more nucleotide positions selected from the group consisting of positions 60-897 of SEQ ID NO: 147.

78. The isolated RNA polynucleotide of any one of claims 1-59, wherein
the virus is Respiratory Syncytial Virus (RSV),
wherein the L region comprises a nucleotide sequence set forth as any one of SEQ ID NOs: 158, 163, 165, 166, or 419; or a variant of any one of SEQ ID NOs: 158, 163, 165, 166, or 419, wherein
(i) the variant of SEQ ID NO: 158 comprises a variation at one or more nucleotide positions selected from the group consisting of positions 15-207 of SEQ ID NO: 158;
(ii) the variant of SEQ ID NO: 163 comprises a variation at one or more nucleotide positions selected from the group consisting of positions 18-210 of SEQ ID NO: 163;
(iii) the variant of SEQ ID NO: 165 comprises a variation at one or more nucleotide positions selected from the group consisting of positions 15-147 of SEQ ID NO: 165;
(iv) the variant of SEQ ID NO: 166 comprises a variation at one or more nucleotide positions selected from the group consisting of positions 15-32 of SEQ ID NO: 166; or
(v) the variant of SEQ ID NO: 419 comprises a variation at one or more nucleotide positions selected from the group consisting of positions 18-35 of SEQ ID NO: 419; and
wherein the R region comprises the nucleotide sequence set forth as any one of SEQ ID NOs: 169, 170, 176, 177, or 420; or a variant of any one of SEQ ID NOs: 169, 170, 176, 177, or 420, wherein
(i) the variant of SEQ ID NO: 169 comprises a variation at one or more nucleotide positions selected from the group consisting of positions 15-78 of SEQ ID NO: 169;
(ii) the variant of SEQ ID NO: 170 comprises a variation at one or more nucleotide positions selected from the group consisting of positions 15-80 of SEQ ID NO: 170;
(iii) the variant of SEQ ID NO: 176 comprises a variation at one or more nucleotide positions selected from the group consisting of positions 15-36 of SEQ ID NO: 176;
(iv) the variant of SEQ ID NO: 177 comprises a variation at one or more nucleotide positions selected from the group consisting of positions 15-33 of SEQ ID NO: 177; or
(v) the variant of SEQ ID NO: 420 comprises a variation at one or more nucleotide positions selected from the group consisting of positions 15-35 of SEQ ID NO: 420.

79. The isolated RNA polynucleotide of any one of claims 1-59, wherein
the virus is a parainfluenzavirus,
wherein the L region comprises a nucleotide sequence set forth as any one of SEQ ID NOs: 181, 182, or 183; or a variant of any one of SEQ ID NOs: 181, 182, or 183, wherein
(i) the variant of SEQ ID NO: 181 comprises a variation at one or more nucleotide positions selected from the group consisting of positions 17-136 of SEQ ID NO: 181;
(ii) the variant of SEQ ID NO: 182 comprises a variation at one or more nucleotide positions selected from the group consisting of positions 17-142 of SEQ ID NO: 182; or
(iii) the variant of SEQ ID NO: 183 comprises a variation at one or more nucleotide positions selected from the group consisting of positions 17-136 of SEQ ID NO: 183; and
wherein the R region comprises the nucleotide sequence set forth as SEQ ID NO: 184; or a variant of SEQ ID NO: 184, wherein the variant of SEQ ID NO: 184 comprises a variation at one or more nucleotide positions selected from the group consisting of positions 17-98 of SEQ ID NO: 184.

80. The isolated RNA polynucleotide of any one of claims 1-59, wherein
the virus is a parainfluenzavirus,
wherein the L region comprises a nucleotide sequence set forth as any one of SEQ ID NOs: 187, 188, or 189; or a variant of any one of SEQ ID NOs: 187, 188, or 189, wherein
(i) the variant of SEQ ID NO: 187 comprises a variation at one or more nucleotide positions selected from the group consisting of positions 21-95 of SEQ ID NO: 187;
(ii) the variant of SEQ ID NO: 188 comprises a variation at one or more nucleotide positions selected from the group consisting of positions 21-101 of SEQ ID NO: 188; or
(iii) the variant of SEQ ID NO: 189 comprises a variation at one or more nucleotide positions selected from the group consisting of positions 21-95 of SEQ ID NO: 189;
and
wherein the R region comprises the nucleotide sequence set forth as SEQ ID NO: 190; or a variant of SEQ ID NO: 190, wherein the variant of SEQ ID NO: 190 comprises a variation at one or more nucleotide positions selected from the group consisting of positions 21-93 of SEQ ID NO: 190.

81. The isolated RNA polynucleotide of any one of claims 1-59, wherein
the virus is a metapneumovirus,
wherein the L region comprises a nucleotide sequence set forth as any one of SEQ ID NOs: 196, 197, or 199; or a variant of any one of SEQ ID NOs: 196, 197, or 199, wherein
(i) the variant of SEQ ID NO: 196 comprises a variation at one or more nucleotide positions selected from the group consisting of positions 21-224 of SEQ ID NO: 196;
(ii) the variant of SEQ ID NO: 197 comprises a variation at one or more nucleotide positions selected from the group consisting of positions 21-230 of SEQ ID NO: 197;
(iii) the variant of SEQ ID NO: 199 comprises a variation at one or more nucleotide positions selected from the group consisting of positions 21-140 of SEQ ID NO: 199;
and
wherein the R region comprises the nucleotide sequence set forth as SEQ ID NO: 201; or a variant of SEQ ID NO: 201, wherein the variant of SEQ ID NO: 201 comprises a variation at one or more nucleotide positions selected from the group consisting of positions 17-32 of SEQ ID NO: 201.

82. The isolated RNA polynucleotide of any one of claims 1-59, wherein
the virus is a metapneumovirus,
wherein the L region comprises a nucleotide sequence set forth as SEQ ID NO: 195; or a variant of SEQ ID NO: 195, wherein the variant of SEQ ID NO: 195 comprises a variation at one or more nucleotide positions selected from the group consisting of positions 21-224 of SEQ ID NO: 195; and
wherein the R region comprises the nucleotide sequence set forth as SEQ ID NO: 200; or a variant of SEQ ID NO: 200, wherein the variant of SEQ ID NO: 200 comprises a variation at one or more nucleotide positions selected from the group consisting of positions 17-32 of SEQ ID NO: 200.

83. The isolated RNA polynucleotide of any one of claims 1-59, wherein
the virus is a henipavirus,
wherein the L region comprises a nucleotide sequence set forth as SEQ ID NO: 204; or a variant of SEQ ID NO: 204, wherein the variant of SEQ ID NO: 204 comprises a variation at one or more nucleotide positions selected from the group consisting of positions 17-77 of SEQ ID NO: 204; and
wherein the R region comprises the nucleotide sequence set forth as SEQ ID NO: 206; or a variant of SEQ ID NO: 206, wherein the variant of SEQ ID NO: 206 comprises a variation at one or more nucleotide positions selected from the group consisting of positions 17-91 of SEQ ID NO: 206.

84. The isolated RNA polynucleotide of any one of claims 1-59, wherein
the virus is a henipavirus,
wherein the L region comprises a nucleotide sequence set forth as SEQ ID NO: 209 or 210; or a variant of SEQ ID NO: 209 or 210, wherein
(i) the variant of SEQ ID NO: 209 comprises a variation at one or more nucleotide positions selected from the group consisting of positions 17-77 of SEQ ID NO: 209; or
(ii) the variant of SEQ ID NO: 210 comprises a variation at one or more nucleotide positions selected from the group consisting of positions 17-83 of SEQ ID NO: 210; and
wherein the R region comprises the nucleotide sequence set forth as SEQ ID NO: 211; or a variant of SEQ ID NO: 211, wherein the variant of SEQ ID NO: 211 comprises a variation at one or more nucleotide positions selected from the group consisting of positions 17-91 of SEQ ID NO: 211.

85. The isolated RNA polynucleotide of any one of claims 1-59, wherein
the virus is a hepadnavirus,
wherein the L region comprises a nucleotide sequence set forth as SEQ ID NO: 222 or 223; or a variant of SEQ ID NO: 222 or 223, wherein
(i) the variant of SEQ ID NO: 222 comprises a variation at one or more nucleotide positions selected from the group consisting of positions 101-639 of SEQ ID NO: 222; or
(ii) the variant of SEQ ID NO: 223 comprises a variation at one or more nucleotide positions selected from the group consisting of positions 101-186 of SEQ ID NO: 223; and
wherein the R region comprises the nucleotide sequence set forth as SEQ ID NO: 225; or a variant of SEQ ID NO: 225, wherein the variant of SEQ ID NO: 225 comprises a variation at one or more nucleotide positions selected from the group consisting of positions 101-1023 of SEQ ID NO: 225.

86. The isolated RNA polynucleotide of any one of claims 1-59, wherein
the virus is a filovirus,
wherein the L region comprises a nucleotide sequence set forth as any one of SEQ ID NOs: 227, 228, 229, or 230; or a variant of any one of SEQ ID NOs: 227, 228, 229, or 230, wherein
(i) the variant of SEQ ID NO: 227 comprises a variation at one or more nucleotide positions selected from the group consisting of positions 20-710 of SEQ ID NO: 227;
(ii) the variant of SEQ ID NO: 228 comprises a variation at one or more nucleotide positions selected from the group consisting of positions 23-713 of SEQ ID NO: 228;
(iii) the variant of SEQ ID NO: 229 comprises a variation at one or more nucleotide positions selected from the group consisting of positions 17-707 of SEQ ID NO: 229; or
(iv) the variant of SEQ ID NO: 230 comprises a variation at one or more nucleotide positions selected from the group consisting of positions 17-707 of SEQ ID NO: 230; and
wherein the R region comprises the nucleotide sequence set forth as SEQ ID NO: 231; or a variant of SEQ ID NO: 231, wherein the variant of SEQ ID NO: 231 comprises a variation at one or more nucleotide positions selected from the group consisting of positions 15-449 of SEQ ID NO: 231.

87. The isolated RNA polynucleotide of any one of claims 1-59, wherein
the virus is a filovirus,
wherein the L region comprises a nucleotide sequence set forth as any one of SEQ ID NOs: 232, 233, 234, or 235; or a variant of any one of SEQ ID NOs: 232, 233, 234, or 235, wherein
(i) the variant of SEQ ID NO: 232 comprises a variation at one or more nucleotide positions selected from the group consisting of positions 20-678 of SEQ ID NO: 232;
(ii) the variant of SEQ ID NO: 233 comprises a variation at one or more nucleotide positions selected from the group consisting of positions 23-681 of SEQ ID NO: 233;
(iii) the variant of SEQ ID NO: 234 comprises a variation at one or more nucleotide positions selected from the group consisting of positions 23-678 of SEQ ID NO: 234; or
(iv) the variant of SEQ ID NO: 235 comprises a variation at one or more nucleotide positions selected from the group consisting of positions 23-678 of SEQ ID NO: 235; and
wherein the R region comprises the nucleotide sequence set forth as SEQ ID NO: 236; or a variant of SEQ ID NO: 236, wherein the variant of SEQ ID NO: 236 comprises a variation at one or more nucleotide positions selected from the group consisting of positions 15-437 of SEQ ID NO: 236.

88. The isolated RNA polynucleotide of any one of claims 1-59, wherein
the virus is a filovirus,
wherein the L region comprises a nucleotide sequence set forth as any one of SEQ ID NOs: 237, 238, or 239; or a variant of any one of SEQ ID NOs: 237, 238, or 239, wherein
(i) the variant of SEQ ID NO: 237 comprises a variation at one or more nucleotide positions selected from the group consisting of positions 20-605 of SEQ ID NO: 237;
(ii) the variant of SEQ ID NO: 238 comprises a variation at one or more nucleotide positions selected from the group consisting of positions 21-606 of SEQ ID NO: 238; or
(iii) the variant of SEQ ID NO: 239 comprises a variation at one or more nucleotide positions selected from the group consisting of positions 20-605 of SEQ ID NO: 239; and
wherein the R region comprises the nucleotide sequence set forth as SEQ ID NO: 240; or a variant of SEQ ID NO: 240, wherein the variant of SEQ ID NO: 240 comprises a variation at one or more nucleotide positions selected from the group consisting of positions 15-83 of SEQ ID NO: 240.

89. The isolated RNA polynucleotide of any one of claims 1-59, wherein
the virus is a filovirus,
wherein the L region comprises a nucleotide sequence set forth as SEQ ID NO: 241; or a variant of SEQ ID NO: 241, wherein the variant of SEQ ID NO: 241 comprises a variation at one or more nucleotide positions selected from the group consisting of positions 20-34 of SEQ ID NO: 241; and
wherein the R region comprises the nucleotide sequence set forth as SEQ ID NO: 242; or a variant of any one of SEQ ID NO: 242, wherein the variant of SEQ ID NO: 242 comprises a variation at one or more nucleotide positions selected from the group consisting of positions 100-593 of SEQ ID NO: 242.

90. The isolated RNA polynucleotide of any one of claims 1-59, wherein
the virus is a filovirus,
wherein the L region comprises a nucleotide sequence set forth as SEQ ID NO: 243; or a variant of SEQ ID NO: 243, wherein the variant of SEQ ID NO: 243 comprises a variation at one or more nucleotide positions selected from the group consisting of positions 30-45 of SEQ ID NO: 243; and
wherein the R region comprises the nucleotide sequence set forth as SEQ ID NO: 244; or a variant of SEQ ID NO: 244, wherein the variant of SEQ ID NO: 244 comprises a variation at one or more nucleotide positions selected from the group consisting of positions 100-677 of SEQ ID NO: 244.

91. The isolated RNA polynucleotide of any one of claims 1-59, wherein
the virus is a filovirus,
wherein the L region comprises a nucleotide sequence set forth as any one of SEQ ID NOs: 245, 246, or 247; or a variant of any one of SEQ ID NOs: 245, 246, or 247, wherein
(i) the variant of SEQ ID NO: 245 comprises a variation at one or more nucleotide positions selected from the group consisting of positions 29-171 of SEQ ID NO: 245;
(ii) the variant of SEQ ID NO: 246 comprises a variation at one or more nucleotide positions selected from the group consisting of positions 30-171 of SEQ ID NO: 246; or
(iii) the variant of SEQ ID NO: 247 comprises a variation at one or more nucleotide positions selected from the group consisting of positions 29-171 of SEQ ID NO: 247; and
wherein the R region comprises the nucleotide sequence set forth as SEQ ID NO: 248; or a variant of SEQ ID NO: 248, wherein the variant of SEQ ID NO: 248 comprises a variation at one or more nucleotide positions selected from the group consisting of positions 20-91 of SEQ ID NO: 248.

92. The isolated RNA polynucleotide of any one of claims 1-59, wherein
the virus is an alphavirus,
wherein the L region comprises a nucleotide sequence set forth as SEQ ID NO: 249; or a variant of SEQ ID NO: 249, wherein the variant of SEQ ID NO: 249 comprises a variation at one or more nucleotide positions selected from the group consisting of positions 60-274 of SEQ ID NO: 249; and
wherein the R region comprises the nucleotide sequence set forth as SEQ ID NO: 250 or 251; or a variant of SEQ ID NO: 250 or 251, wherein
(i) the variant of SEQ ID NO: 250 comprises a variation at one or more nucleotide positions selected from the group consisting of positions 60-183 of SEQ ID NO: 250; or
(ii) the variant of SEQ ID NO: 251 comprises a variation at one or more nucleotide positions selected from the group consisting of positions 60-375 of SEQ ID NO: 251.

93. The isolated RNA polynucleotide of any one of claims 1-59, wherein
the virus is an alphavirus,
wherein the L region comprises a nucleotide sequence set forth as SEQ ID NO: 255; or a variant of SEQ ID NO: 255, wherein the variant of SEQ ID NO: 255 comprises a variation at one or more nucleotide positions selected from the group consisting of positions 60-35 of SEQ ID NO: 255; and
wherein the R region comprises the nucleotide sequence set forth as SEQ ID NO: 256 or 257; or a variant of SEQ ID NO: 256 or 257, wherein
(i) the variant of SEQ ID NO: 256 comprises a variation at one or more nucleotide positions selected from the group consisting of positions 600-273 of SEQ ID NO: 256; or
(ii) the variant of SEQ ID NO: 257 comprises a variation at one or more nucleotide positions selected from the group consisting of positions 60-377 of SEQ ID NO: 257.

94. The isolated RNA polynucleotide of any one of claims 1-59, wherein
the virus is an alphavirus,
wherein the L region comprises a nucleotide sequence set forth as SEQ ID NO: 261; or a variant of SEQ ID NO: 261, wherein the variant of SEQ ID NO: 261 comprises a variation at one or more nucleotide positions selected from the group consisting of positions 60-215 of SEQ ID NO: 261; and
wherein the R region comprises the nucleotide sequence set forth as SEQ ID NO: 262 or 263; or a variant of SEQ ID NO: 262 or 263, wherein
(i) the variant of SEQ ID NO: 262 comprises a variation at one or more nucleotide positions selected from the group consisting of positions 60-166 of SEQ ID NO: 262; or
(ii) the variant of SEQ ID NO: 263 comprises a variation at one or more nucleotide positions selected from the group consisting of positions 60-379 of SEQ ID NO: 263.

95. An isolated RNA polynucleotide, comprising
a coding region having a coding sequence encoding one or more polypeptide; and
template regions, wherein the template regions comprise two distinct regions, a left flanking region (“L region”) of a virus and a right flanking region (“R region”) of the virus, wherein the L region is adjacent to and contiguous with a 5′ end of the coding region and the R region is adjacent to and contiguous with a 3′ end of the coding region;
wherein the coding sequence is in a sense orientation;
wherein the polypeptide is heterologous to the virus;
wherein the template region interact with and initiate RNA-dependent polymerase activity of a polymerase in a cell containing the RNA dependent polymerase; and
wherein the virus is not an alphavirus.

96. The reverse complement of the isolated RNA polynucleotide of claim 95.

97. The isolated RNA polynucleotide of claim 95 or 96, wherein the virus is selected from the group consisting of viruses in the orders of Amarillovirales, Articulavirales, Blubervirales, Bunyavirales, Hepelivirales, Mononegavirales, Nidovirales, and Picornavirales.

98. The isolated RNA polynucleotide of any one of claims 95-97, wherein the virus is selected from the group consisting of viruses in the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, and Rhabdoviridae.

99. The isolated RNA polynucleotide of any one of claims 95-98, wherein the virus is from the group consisting of Alphacoronavirus 229E, Alphacoronavirus NL63, Alphacoronavirus WA2028, Avian metapneumovirus (AMPV), Betacoronavirus HKU1, Betacoronavirus HKU15, Betacoronavirus HKU33, Betacoronavirus OC43, Chikungunya virus, Crimean-Congo Hemorrhagic Fever Virus, Dengue Virus, Enterovirus D68 (EV-D68), Foot and Mouth Disease Virus, Hanta Virus, Hendra Virus, Hepatitis B Virus, Hepatitis C Virus, HMPV, Human Parainfluenzavirus 1 (HPIV1), Human Parainfluenzavirus 3 (HPIV3), Infectious Salmon Anemia Virus, Influenza A Virus, Influenza B Virus, Lassa Virus, Marburg Virus, Middle East Respiratory Syndrome Coronavirus (MERS-COV), Newcastle Disease Virus (NDV), Nipah Virus, Norwalk Virus, Rabies Virus, Respiratory Syncytial Virus, Reston Ebola virus, Rhinovirus, Rift Valley Fever Virus, Rubella virus, SARS-COV-1, SARS-COV-2, Sudan Ebola virus, Vesicular Stomatitis Virus, Yellow Fever Virus, Zaire Ebola virus, and Zika Virus.

100. The isolated RNA polynucleotide of any one of claims 95-99, wherein the template regions are native to the virus.

101. The isolated RNA polynucleotide of any one of claims 95-99, wherein the template regions are variants of template regions native to the virus, wherein the variants have at least 90%, 91%, 92%, 93%, 94%, 95%, 96%, 97%, 98%, or 99% sequence identity to the template regions native to the virus.

102. The isolated RNA polynucleotide of any one of claims 95-101, wherein each of the L and the R regions of the template regions comprise fewer than 10, 9, 8, 7, 6, 5, 4, 3, or 2 variations relative to template regions native to the virus.

103. The isolated RNA polynucleotide of any one of claims 95-101, wherein each of the L and the R regions of the template regions vary from template regions native to the virus by not more than 10, 9, 8, 7, 6, 5, 4, 3, or 2 substitutions that are not involved in 5′ capping.

104. The isolated RNA polynucleotide of any one of claims 95-103, wherein each of the L and the R regions of the template regions varies from template regions native to the virus by not more than 1 substitution that is not involved in 5′ capping.

105. The isolated RNA polynucleotide of any one of claims 95-104, wherein the isolated RNA polynucleotide comprises at least one nucleoside modification.

106. The isolated RNA polynucleotide of any one of claims 95-105, wherein the template regions are nucleoside modified, wherein the percentage of modified nucleosides is not more than 40%, 35%, 30%, 25%, 20%, 15%, 10%, or 5%.

107. The isolated RNA polynucleotide of any one of claims 95-105, wherein the template regions are nucleoside modified, wherein the percentage of modified nucleosides is at least 30%, 35%, 40%, 45%, 50%, 55%, 60%, 65%, 70%, 75%, 80%, 90%, 95%, or 100%.

108. The isolated RNA polynucleotide of claim 105 or 106, wherein the nucleoside modification is a nonimmunogenic uridine modification, and the percentage of modified uridine modifications is not more than 40%, 35%, 30%, 25%, 20% 15% or 10%.

109. The isolated RNA polynucleotide of claim 105 or 107, wherein the nucleoside modification is a nonimmunogenic uridine modification, and the percentage of modified uridine modifications is more than 30%, 35%, 40%, 45%, 50%, 55%, 60%, 65%, 70%, 75%, 80%, 90%, or 95%, or is 100%.

110. The isolated RNA polynucleotide of any one of claim 105 or 106, wherein the nucleoside modification is a nonimmunogenic cytidine modification, and the percentage of modified cytidine modifications is not more than 40%, 35%, 30%, 25%, 20% 15% or 10%.

111. The isolated RNA polynucleotide of any one of claim 105 or 107, wherein the nucleoside modification is a nonimmunogenic cytidine modification, and the percentage of modified cytidine modifications is more than 30%, 35%, 40%, 45%, 50%, 55%, 60%, 65%, 70%, 75%, 80%, 90%, or 95%, or is 100%.

112. The isolated RNA polynucleotide of claim 105 or 106, wherein the nucleoside modification is a nonimmunogenic adenosine modification, and the percentage of modified adenosine modifications is between 1% and 30%.

113. The isolated RNA polynucleotide of claim 112, wherein the nucleoside modification is a nonimmunogenic adenosine modification, and the percentage of modified adenosine modifications is about 1%, 5%, 10%, 15%, 20%, 25%, or 30%.

114. The isolated RNA polynucleotide of any one of claims 95-113, wherein the isolated

115. The isolated RNA polynucleotide of any one of claims 95-114, wherein the 5′ end of the L region comprises a 5′ cap structure.

116. The isolated RNA polynucleotide of any one of claims 95-115, wherein the 5′ end of the L region comprises one or more variations associated with a 5′ cap structure.

117. The isolated RNA polynucleotide of any one of claims 114-116, wherein the 5′-cap structure is selected from the group consisting of Cap 0, Cap 0 (3′-O-Me), Cap 1, Cap 1 (3′-O-Me), Cap 2, Cap 2 (3′-O-Me), Anti-Reverse Cap Analog (ARCA), inosine, N1-methyl-guanosine, 2′-fluoro-guanosine, 7-deaza-guanosine, 8-oxo-guanosine, 2-amino-guanosine, locked nucleic acid guanosine (LNA-guanosine), and 2-azido-guanosine structure.

118. The isolated RNA polynucleotide of any one of claims 95-113, wherein the isolated polynucleotide does not comprise a 5′ cap structure (uncapped).

119. The isolated RNA polynucleotide of any one of claim 95-113 or 118, wherein the 5′ end of the L region does not comprise a 5′ cap structure (uncapped).

120. The isolated RNA polynucleotide of claim 118 or 119, wherein the 5′ end of the isolated polynucleotide comprises a 5′-monophosphate, 5′-diphosphate, or 5′-triphosphate.

121. The isolated RNA polynucleotide of claim 118 or 119, wherein the 5′ end of the isolated polynucleotide does not comprise a 5′-phosphate (dephosphorylated).

122. The isolated RNA polynucleotide of claim 96, wherein the template regions are the reverse complement of template regions native to the virus.

123. The isolated RNA polynucleotide of claim 96 or 122, wherein the template regions are variants of a reverse complement of template regions native to the virus, wherein the variants have at least 90%, 91%, 92%, 93%, 94%, 95%, 96%, 97%, 98%, or 99% sequence identity to the reverse complement of the template regions native to the virus.

124. The isolated RNA polynucleotide of claim 122 or 123, wherein the reverse complements of each of the L and the R regions vary from the reverse complements of template regions native to the virus by not more than 10, 9, 8, 7, 6, 5, 4, 3, or 2 substitutions that are not involved in 5′ capping.

125. The isolated RNA polynucleotide of claim 123 or 124, wherein the reverse complements of each of the L and the R regions vary from the reverse complements of template regions native to the virus by not more than 1 substitution that is not involved in 5′ capping.

126. The isolated RNA polynucleotide of any one of claims 122-125, wherein the isolated RNA polynucleotide comprises at least one nucleoside modification.

127. The isolated RNA polynucleotide of 126, wherein the template regions are nucleoside-modified and the percentage of modified nucleotides is not more than 40%, 35%, 30%, 25%, 20%, 15%, 10%, or 5%.

128. The isolated RNA polynucleotide of any one of claim 96 or 122-127, wherein the 5′ end of the reverse complement of the R region encodes a cap structure.

129. The isolated RNA polynucleotide of any one of claim 96 or 122-128, wherein the 5′ end of the R region is capped.

130. The isolated RNA polynucleotide of any one of claim 95-129, wherein the therapeutic polypeptide is a secreted polypeptide.

131. The isolated RNA polynucleotide of any one of claim 95-130, wherein the therapeutic polypeptide is selected from the group consisting of an interferon, an interferon stimulated gene, a cytokine, a chemokine, an antibody, a signaling molecule, a cytotoxic protein, a protein that causes cell death, an antineoplastic protein, an immunomodulatory protein, protein toll-like receptor agonist, or a dominant negative protein.

132. The isolated RNA polynucleotide of claim 131, wherein the cytokine is an inflammatory cytokine.

133. The isolated RNA polynucleotide of claim 131, wherein the inflammatory cytokine is TNF-α.

134. The isolated RNA polynucleotide of claim 131, wherein the cytokine is an anti-inflammatory cytokine.

135. The isolated RNA polynucleotide of claim 134, wherein the anti-inflammatory cytokine is an interleukin-1 receptor antagonist (IL-1RN).

136. The isolated RNA polynucleotide of any one of claim 95-135, wherein the therapeutic polypeptide is an interleukin or a caspase.

137. The isolated RNA polynucleotide of claim 136, wherein the interleukin is IL-12A, IL-12B or IL-2.

138. The isolated RNA polynucleotide of claim 131, wherein the therapeutic polypeptide is an antibody.

139. The isolated RNA polynucleotide of claim 131, wherein the therapeutic polypeptide is an interferon.

140. The isolated RNA polynucleotide of claim 139, wherein the interferon is an IFN-α, IFN-β, IFN-ε, IFN-κ, IFN-ω, IFN-γ, or IFN-λ.

141. The isolated RNA polynucleotide of claim 140, wherein the interferon is IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-β1, IFN-ε, IFN-κ, IFN-ω1, IFN-γ, IFN-λ1 (IL28A), IFN-λ2 (IL28B), IFN-λ3 (IL29), or IFN-λ4.

142. The isolated RNA of claim 139, wherein the interferon is IFN-α, IFN-β, IFN-κ, IFN-λ1 (IL28A), IFN-λ2 (IL28B), or IFN-λ3 (IL29).

143. The isolated RNA polynucleotide of any one of claim 95-142, wherein the coding sequence encodes more than one therapeutic polypeptide, which are separated by one or more ribosomal skipping sequence.

144. The isolated RNA polynucleotide of any one of claims 95-143, wherein the coding region further comprises one or more regulatory elements selected from the group consisting of ribosomal binding site, Kozak sequence, Shine-Dalgarno sequence, ribozyme, riboswitch, promoter, microRNA binding site, and internal ribosomal entry site (IRES).

145. The isolated RNA polynucleotide of claim 144, wherein the one or more regulatory elements are operably linked to the coding sequence.

146. The isolated RNA polynucleotide of any one of claim 95-145, further comprising a polyadenylation signal and/or a 3′ poly(A) tail.

147. The isolated RNA polynucleotide of any one of claims 95-146, wherein the RNA-dependent polymerase is an RNA-dependent RNA polymerase.

148. The isolated RNA polynucleotide of any one of claims 95-146, wherein the RNA-dependent polymerase is an RNA-dependent DNA polymerase.

149. The isolated RNA polynucleotide of any one of claims 95-148, wherein the RNA-dependent polymerase is a polymerase is from the virus.

150. The isolated RNA polynucleotide of any one of claims 95-149, wherein the isolated RNA polynucleotide is a single stranded RNA.

151. The isolated RNA polynucleotide of any one of claims 95-150, wherein the isolated polynucleotide is in linear form.

152. The isolated RNA polynucleotide of any one of claims 95-150, wherein the isolated polynucleotide is in a covalently-closed circular form.

153. The isolated RNA polynucleotide of any one of claims 95-152, wherein the virus is a sarbecovirus,
wherein the L region comprises a nucleotide sequence set forth as any one of SEQ ID NOs: 60, 61, 62, 63, 64, 65, 66, or 67; or a variant of any one of SEQ ID NOs: 60, 61, 62, 63, 64, 65, 66, or 67, wherein the variant comprises a variation at one or more nucleotide positions selected from the group consisting of positions 39-789 or 1426-1493 of any one of SEQ ID NOs: 60, 61, 62, 63, 64, 65, 66, or 67; and
wherein the R region comprises the nucleotide sequence set forth SEQ ID NO: 129; or a variant of SEQ ID NO: 129, wherein the variant of SEQ ID NO: 129 comprises a variation at one or more nucleotide positions selected from the group consisting of positions 20-320 of SEQ ID NO: 129.

154. The isolated RNA polynucleotide of any one of claims 95-152, wherein
the virus is a sarbecovirus,
wherein the L region comprises a nucleotide sequence set forth as any one of SEQ ID NOs: 68, 69, 70, 71, 72, 73, 74, 75, 76, or 77; or a variant of any one of SEQ ID NOW: 68, 69, 70, 71, 72, 73, 74, 75, 76, or 77, wherein
(i) the variant of SEQ ID NO: 68 comprises a variation at one or more nucleotide positions selected from the group consisting of positions 39-789 or 1434-1501 of SEQ ID NO: 68;
(ii) the variant of SEQ ID NO: 69 comprises a variation at one or more nucleotide positions selected from the group consisting of positions 39-789 or 1434-1501 of SEQ ID NO: 69;
(iii) the variant of SEQ ID NO: 70 comprises a variation at one or more nucleotide positions selected from the group consisting of positions 39-789 or 1446-1513 of SEQ ID NO: 70;
(iv) the variant of SEQ ID NO: 71 comprises a variation at one or more nucleotide positions selected from the group consisting of positions 39-789 or 1455-1522 of SEQ ID NO: 71;
(v) the variant of SEQ ID NO: 72 comprises a variation at one or more nucleotide positions selected from the group consisting of positions 39-789 or 1462-1529 of SEQ ID NO: 72;
(vi) the variant of SEQ ID NO: 73 comprises a variation at one or more nucleotide positions selected from the group consisting of positions 39-789 or 1469-1536 of SEQ ID NO: 73;
(vii) the variant of SEQ ID NO: 74 comprises a variation at one or more nucleotide positions selected from the group consisting of positions 39-789 or 1485-1552 of SEQ ID NO: 74;
(viii) the variant of SEQ ID NO: 75 comprises a variation at one or more nucleotide positions selected from the group consisting of positions 39-789 or 1686-1753 of SEQ ID NO: 75;
(ix) the variant of SEQ ID NO: 76 comprises a variation at one or more nucleotide positions selected from the group consisting of positions 39-789 or 1704-1771 of SEQ ID NO: 76; or
(x) the variant of SEQ ID NO: 77 comprises a variation at one or more nucleotide positions selected from the group consisting of positions 39-789 or 1720-1787 of SEQ ID NO: 77; and
wherein the R region comprises the nucleotide sequence set forth as SEQ ID NO: 130; or a variant of SEQ ID NO: 130, wherein the variant of SEQ ID NO: 130 comprises a variation at one or more nucleotide positions selected from the group consisting of positions 20-320 of SEQ ID NO: 130.

155. The isolated RNA polynucleotide of any one of claims 95-152, wherein
the virus is a sarbecovirus,
wherein the L region comprises a nucleotide sequence set forth as any one of SEQ ID NOs: 78, 79, 80, 81, 82, 83, 85, 86, 87, or 88; or a variant of any one of SEQ ID NOs: 78, 79, 80, 81, 82, 83, 85, 86, 87, or 88, wherein
(i) the variant of SEQ ID NO: 78 comprises a variation at one or more nucleotide positions selected from the group consisting of positions 39-789 or 1734-1801 of SEQ ID NO: 78;
(ii) the variant of SEQ ID NO: 79 comprises a variation at one or more nucleotide positions selected from the group consisting of positions 39-789 or 1687-1754 of SEQ ID NO: 79;
(iii) the variant of SEQ ID NO: 80 comprises a variation at one or more nucleotide positions selected from the group consisting of positions 39-789 or 1695-1762 of SEQ ID NO: 80;
(iv) the variant of SEQ ID NO: 81 comprises a variation at one or more nucleotide positions selected from the group consisting of positions 39-789 or 1434-1501 of SEQ ID NO: 81;
(v) the variant of SEQ ID NO: 82 comprises a variation at one or more nucleotide positions selected from the group consisting of positions 39-789 or 1443-1510 of SEQ ID NO: 82;
(vi) the variant of SEQ ID NO: 83 comprises a variation at one or more nucleotide positions selected from the group consisting of positions 39-789 or 1459-1526 of SEQ ID NO: 83;
(vii) the variant of SEQ ID NO: 85 comprises a variation at one or more nucleotide positions selected from the group consisting of positions 39-789 or 1434-1501 of SEQ ID NO: 85;
(viii) the variant of SEQ ID NO: 86 comprises a variation at one or more nucleotide positions selected from the group consisting of positions 39-789 or 1434-1501 of SEQ ID NO: 86;
(ix) the variant of SEQ ID NO: 87 comprises a variation at one or more nucleotide positions selected from the group consisting of positions 39-789 or 1435-1502 of SEQ ID NO: 87; or
(x) the variant of SEQ ID NO: 88 comprises a variation at one or more nucleotide positions selected from the group consisting of positions 39-789 or 1463-1530 of SEQ ID NO: 88; and
wherein the R region comprises the nucleotide sequence set forth as SEQ ID NO: 130; or a variant of SEQ ID NO: 130, wherein the variant of SEQ ID NO: 130 comprises a variation at one or more nucleotide positions selected from the group consisting of positions 20-320 of SEQ ID NO: 130.

156. The isolated RNA polynucleotide of any one of claims 95-152, wherein
the virus is a sarbecovirus,
wherein the L region comprises a nucleotide sequence set forth as any one of SEQ ID NOs: 89, 90, 91, 92, 96, 104, 105, 106, 107, or 108; or a variant of any one of SEQ ID NOs: 89, 90, 91, 92, 96, 104, 105, 106, 107, or 108, wherein
(i) the variant of SEQ ID NO: 89 comprises a variation at one or more nucleotide positions selected from the group consisting of positions 39-789 or 1466-1533 of SEQ ID NO: 89;
(ii) the variant of SEQ ID NO: 90 comprises a variation at one or more nucleotide positions selected from the group consisting of positions 39-789 or 1425-1492 of SEQ ID NO: 90;
(iii) the variant of SEQ ID NO: 91 comprises a variation at one or more nucleotide positions selected from the group consisting of positions 39-789 or 1425-1492 of SEQ ID NO: 91;
(iv) the variant of SEQ ID NO: 92 comprises a variation at one or more nucleotide positions selected from the group consisting of positions 39-789 or 1425-1492 of SEQ ID NO: 92;
(v) the variant of SEQ ID NO: 96 comprises a variation at one or more nucleotide positions selected from the group consisting of positions 39-769 or 1471-1471 of SEQ ID NO: 96;
(vi) the variant of SEQ ID NO: 104 comprises a variation at one or more nucleotide positions selected from the group consisting of positions 39-789 or 1446-1513 of SEQ ID NO: 104;
(vii) the variant of SEQ ID NO: 105 comprises a variation at one or more nucleotide positions selected from the group consisting of positions 39-789 or 1455-1522 of SEQ ID NO: 105;
(viii) the variant of SEQ ID NO: 106 comprises a variation at one or more nucleotide positions selected from the group consisting of positions 39-789 or 1462-1529 of SEQ ID NO: 106;
(ix) the variant of SEQ ID NO: 107 comprises a variation at one or more nucleotide positions selected from the group consisting of positions 39-789 or 1469-1536 of SEQ ID NO: 107; or
(x) the variant of SEQ ID NO: 108 comprises a variation at one or more nucleotide positions selected from the group consisting of positions 89-839 or 1485-1552 of SEQ ID NO: 108; and
wherein the R region comprises the nucleotide sequence set forth as SEQ ID NO: 130; or a variant of SEQ ID NO: 130, wherein the variant of SEQ ID NO: 130 comprises a variation at one or more nucleotide positions selected from the group consisting of positions 20-320 of SEQ ID NO: 130.

157. The isolated RNA polynucleotide of any one of claims 95-152, wherein
the virus is a sarbecovirus,
wherein the L region comprises a nucleotide sequence set forth as any one of SEQ ID NOs: 109, 110, 111, 112, 113, 114, 115, 116, 117, or 118; or a variant of any one of SEQ ID NOs: 109, 110, 111, 112, 113, 114, 115, 116, 117, or 118, wherein
(i) the variant of SEQ ID NO: 109 comprises a variation at one or more nucleotide positions selected from the group consisting of positions 40-789 or 1686-1753 of SEQ ID NO: 109;
(ii) the variant of SEQ ID NO: 110 comprises a variation at one or more nucleotide positions selected from the group consisting of positions 40-789 or 1704-1771 of SEQ ID NO: 110;
(iii) the variant of SEQ ID NO: 111 comprises a variation at one or more nucleotide positions selected from the group consisting of positions 40-789 or 1720-1787 of SEQ ID NO: 111;
(iv) the variant of SEQ ID NO: 112 comprises a variation at one or more nucleotide positions selected from the group consisting of positions 40-789 or 1734-1801 of SEQ ID NO: 112;
(v) the variant of SEQ ID NO: 113 comprises a variation at one or more nucleotide positions selected from the group consisting of positions 40-789 or 1687-1754 of SEQ ID NO: 113;
(vi) the variant of SEQ ID NO: 114 comprises a variation at one or more nucleotide positions selected from the group consisting of positions 40-789 or 1695-1762 of SEQ ID NO: 114;
(vii) the variant of SEQ ID NO: 115 comprises a variation at one or more nucleotide positions selected from the group consisting of positions 40-789 or 1434-1501 of SEQ ID NO: 115;
(viii) the variant of SEQ ID NO: 116 comprises a variation at one or more nucleotide positions selected from the group consisting of positions 40-789 or 1434-1501 of SEQ ID NO: 116;
(ix) the variant of SEQ ID NO: 117 comprises a variation at one or more nucleotide positions selected from the group consisting of positions 40-789 or 1434-1501 of SEQ ID NO: 117; or
(xl) the variant of SEQ ID NO: 118 comprises a variation at one or more nucleotide positions selected from the group consisting of positions 40-789 or 1434-1501 of SEQ ID NO: 118; and
wherein the R region comprises the nucleotide sequence set forth as SEQ ID NO: 130; or a variant of SEQ ID NO: 130, wherein the variant of SEQ ID NO: 130 comprises a variation at one or more nucleotide positions selected from the group consisting of positions 20-320 of SEQ ID NO: 130.

158. The isolated RNA polynucleotide of any one of claims 95-152, wherein
the virus is a sarbecovirus,
wherein the L region comprises a nucleotide sequence set forth as any one of SEQ ID NOs: 119, 120, 122, 123, 124, 125, 126, or 127; or a variant of any one of SEQ ID NOs: 119, 120, 122, 123, 124, 125, 126, or 127, wherein
(i) the variant of SEQ ID NO: 119 comprises a variation at one or more nucleotide positions selected from the group consisting of positions 40-789 or 1443-1510 of SEQ ID NO: 119;
(ii) the variant of SEQ ID NO: 120 comprises a variation at one or more nucleotide positions selected from the group consisting of positions 40-789 or 1459-1526 of SEQ ID NO: 120;
(iii) the variant of SEQ ID NO: 122 comprises a variation at one or more nucleotide positions selected from the group consisting of positions 40-789 or 1434-1501 of SEQ ID NO: 122;
(iv) the variant of SEQ ID NO: 123 comprises a variation at one or more nucleotide positions selected from the group consisting of positions 40-789 or 1434-1501 of SEQ ID NO: 123;
(v) the variant of SEQ ID NO: 124 comprises a variation at one or more nucleotide positions selected from the group consisting of positions 40-789 or 1434-1501 of SEQ ID NO: 124;
(vi) the variant of SEQ ID NO: 125 comprises a variation at one or more nucleotide positions selected from the group consisting of positions 40-789 or 1463-1530 of SEQ ID NO: 125;
(vii) the variant of SEQ ID NO: 126 comprises a variation at one or more nucleotide positions selected from the group consisting of positions 40-789 or 1466-1533 of SEQ ID NO: 126;
(viii) the variant of SEQ ID NO: 127 comprises a variation at one or more nucleotide positions selected from the group consisting of positions 40-789 or 1425-1492 of SEQ ID NO: 127; and
wherein the R region comprises the nucleotide sequence set forth as SEQ ID NO: 130; or a variant of SEQ ID NO: 130, wherein the variant of SEQ ID NO: 130 comprises a variation at one or more nucleotide positions selected from the group consisting of positions 20-320 of SEQ ID NO: 130.

159. The isolated RNA polynucleotide of any one of claims 95-152, wherein the virus is a Respiratory Syncytial Virus (RSV),
wherein the L region comprises a nucleotide sequence set forth as any one of SEQ ID NOs: 148, 149, 150, 151, or 152; or a variant of any one of SEQ ID NOs: 148, 149, 150, 151, or 152, wherein
(i) the variant of SEQ ID NO: 148 comprises a variation at one or more nucleotide positions selected from the group consisting of positions 15-78 of SEQ ID NO: 148;
(ii) the variant of SEQ ID NO: 149 comprises a variation at one or more nucleotide positions selected from the group consisting of positions 15-33 of SEQ ID NO: 149;
(iii) the variant of SEQ ID NO: 150 comprises a variation at one or more nucleotide positions selected from the group consisting of positions 15-35 of SEQ ID NO: 150;
(iv) the variant of SEQ ID NO: 151 comprises a variation at one or more nucleotide positions selected from the group consisting of positions 18-36 of SEQ ID NO: 151; or
(v) the variant of SEQ ID NO: 152 comprises a variation at one or more nucleotide positions selected from the group consisting of positions 15-38 of SEQ ID NO: 152;
and
wherein the R region comprises the nucleotide sequence set forth as SEQ ID NO: 154 or 155; or a variant of SEQ ID NO: 154 or 155, wherein
(i) the variant of SEQ ID NO: 154 comprises a variation at one or more nucleotide positions selected from the group consisting of positions 15-207 of SEQ ID NO: 154; or
(ii) the variant of SEQ ID NO: 155 comprises a variation at one or more nucleotide positions selected from the group consisting of positions 15-32 of SEQ ID NO: 155.

160. The isolated RNA polynucleotide of any one of claims 95-152, wherein
the virus is a parainfluenzavirus,
wherein the L region comprises a nucleotide sequence set forth as SEQ ID NO: 180; or a variant of SEQ ID NO: 180, wherein the variant of SEQ ID NO: 180 comprises a variation at one or more nucleotide positions selected from the group consisting of positions 17-136 of SEQ ID NO: 180; and
wherein the R region comprises the nucleotide sequence set forth as SEQ ID NO: 179; or a variant of SEQ ID NO: 179, wherein the variant of SEQ ID NO: 179 comprises a variation at one or more nucleotide positions selected from the group consisting of positions 17-98 of SEQ ID NO: 179.

161. The isolated RNA polynucleotide of any one of claims 95-152, wherein
the virus is a parainfluenzavirus,
wherein the L region comprises a nucleotide sequence set forth as SEQ ID NO: 186; or a variant of SEQ ID NO: 186, wherein the variant of SEQ ID NO: 186 comprises a variation at one or more nucleotide positions selected from the group consisting of positions 21-95 of SEQ ID NO: 186; and
wherein the R region comprises the nucleotide sequence set forth as SEQ ID NO: 185; or a variant of SEQ ID NO: 185, wherein the variant of SEQ ID NO: 185 comprises a variation at one or more nucleotide positions selected from the group consisting of positions 21-93 of SEQ ID NO: 185.

162. The isolated RNA polynucleotide of any one of claims 95-152, wherein
the virus is a metapneumovirus,
wherein the L region comprises a nucleotide sequence set forth as SEQ ID NO: 194; or a variant of SEQ ID NO: 194, wherein the variant of SEQ ID NO: 194 comprises a variation at one or more nucleotide positions selected from the group consisting of positions 17-220 of SEQ ID NO: 194; and
wherein the R region comprises the nucleotide sequence set forth as SEQ ID NO: 192; or a variant of any one of SEQ ID NO: 192, wherein the variant of SEQ ID NO: 192 comprises a variation at one or more nucleotide positions selected from the group consisting of positions 17-32 of SEQ ID NO: 192.

163. The isolated RNA polynucleotide of any one of claims 95-152, wherein
the virus is a metapneumovirus,
wherein the L region comprises a nucleotide sequence set forth as SEQ ID NO: 193; or a variant of SEQ ID NO: 193, wherein the variant of SEQ ID NO: 193 comprises a variation at one or more nucleotide positions selected from the group consisting of positions 17-220 of SEQ ID NO: 193; and
wherein the R region comprises the nucleotide sequence set forth as SEQ ID NO: 191; or a variant of SEQ ID NO: 191, wherein the variant of SEQ ID NO: 191 comprises a variation at one or more nucleotide positions selected from the group consisting of positions 17-32 of SEQ ID NO: 191.

164. The isolated RNA polynucleotide of any one of claims 95-152, wherein
the virus is a henipavirus,
wherein the L region comprises a nucleotide sequence set forth as SEQ ID NO: 203; or a variant of SEQ ID NO: 203, wherein the variant of SEQ ID NO: 203 comprises a variation at one or more nucleotide positions selected from the group consisting of positions 17-77 of SEQ ID NO: 203; and
wherein the R region comprises the nucleotide sequence set forth as SEQ ID NO: 202; or a variant of SEQ ID NO: 202, wherein the variant of SEQ ID NO: 202 comprises a variation at one or more nucleotide positions selected from the group consisting of positions 17-91 of SEQ ID NO: 202.

165. The isolated RNA polynucleotide of any one of claims 95-152, wherein
the virus is a henipavirus,
wherein the L region comprises a nucleotide sequence set forth as SEQ ID NO: 207; or a variant of SEQ ID NO: 207, wherein the variant of SEQ ID NO: 207 comprises a variation at one or more nucleotide positions selected from the group consisting of positions 17-77 of SEQ ID NO: 207; and
wherein the R region comprises the nucleotide sequence set forth as SEQ ID NO: 208; or a variant of SEQ ID NO: 208, wherein the variant of SEQ ID NO: 208 comprises a variation at one or more nucleotide positions selected from the group consisting of positions 17-91 of SEQ ID NO: 208.

166. The isolated RNA polynucleotide of any one of claims 95-152, wherein
the virus is a hepadnavirus,
wherein the L region comprises a nucleotide sequence set forth as any one of SEQ ID NOs: 212, 213, 214, 215, or 216; or a variant of any one of SEQ ID NOs: 212, 213, 214, 215, or 216, wherein
(i) the variant of SEQ ID NO: 212 comprises a variation at one or more nucleotide positions selected from the group consisting of positions 101-1326 of SEQ ID NO: 212;
(ii) the variant of SEQ ID NO: 213 comprises a variation at one or more nucleotide positions selected from the group consisting of positions 101-1291 of SEQ ID NO: 213;
(iii) the variant of SEQ ID NO: 214 comprises a variation at one or more nucleotide positions selected from the group consisting of positions 101-1325 of SEQ ID NO: 214;
(iv) the variant of SEQ ID NO: 215 comprises a variation at one or more nucleotide positions selected from the group consisting of positions 101-15 of SEQ ID NO: 215; or
(v) the variant of SEQ ID NO: 216 comprises a variation at one or more nucleotide positions selected from the group consisting of positions 101-211 of SEQ ID NO: 216; and
wherein the R region comprises the nucleotide sequence set forth as any one of SEQ ID NOs: 217, 218, 219, or 220; or a variant of any one of SEQ ID NOs: 217, 218, 219, or 220, wherein
(i) the variant of SEQ ID NO: 217 comprises a variation at one or more nucleotide positions selected from the group consisting of positions 101-754 of SEQ ID NO: 217;
(ii) the variant of SEQ ID NO: 218 comprises a variation at one or more nucleotide positions selected from the group consisting of positions 101-790 of SEQ ID NO: 218;
(iii) the variant of SEQ ID NO: 219 comprises a variation at one or more nucleotide positions selected from the group consisting of positions 101-892 of SEQ ID NO: 219; or
(iv) the variant of SEQ ID NO: 220 comprises a variation at one or more nucleotide positions selected from the group consisting of positions 101-2309 of SEQ ID NO: 220.

167. An isolated RNA polynucleotide, comprising
a coding region having a coding sequence encoding one or more polypeptides; and
template regions, wherein the template regions comprise two distinct regions, a left flanking region (“L region”) of a virus and a right flanking region (“R region”) of the virus, wherein the L region is adjacent to and contiguous with a 5′ end of the coding region and the R region is adjacent to and contiguous with a 3′ end of the coding region; wherein
at least 30% of uridine nucleotides are modified,
at least 30% of cytidine nucleotides are modified, and/or
between 1-30% of adenosine nucleotides are modified; and
wherein the template region interact with and initiate RNA-dependent polymerase activity of a polymerase in a cell containing the RNA dependent polymerase.

168. The isolated RNA polynucleotide of claim 167, wherein the coding sequence is in an antisense orientation.

169. The isolated RNA polynucleotide of claim 167, wherein the coding sequence is in a sense orientation.

170. The isolated RNA polynucleotide of any one of claims 167-169, wherein polypeptide is a secreted protein.

171. The isolated RNA polynucleotide of any one of claims 167-170, wherein the polypeptide is selected from the group consisting of a medicament, a therapeutic polypeptide, an antigen, and a reporter.

172. The reverse complement of the isolated RNA polynucleotide of any one of claims 167-171.

173. The isolated RNA polynucleotide of any one of claims 167-172, wherein the virus is selected from the group consisting of viruses in the orders of Amarillovirales, Articulavirales, Blubervirales, Bunyavirales, Hepelivirales, Martellivirales, Mononegavirales, Nidovirales, and Picornavirales.

174. The isolated RNA polynucleotide of any one of claims 167-173, wherein the virus is selected from the group consisting of viruses in the families of Arenaviridae, Coronaviridae, Filoviridae, Flaviviridae, Hantaviridae, Hepadnaviridae, Matonaviridae, Nairoviridae, Orthomyxoviridae, Paramyxoviridae, Phenuiviridae, Picornaviridae, Pneumoviridae, Rhabdoviridae, and Togaviridae.

175. The isolated RNA polynucleotide of any one of claims 167-174, wherein the virus is from the group consisting of Alphacoronavirus 229E, Alphacoronavirus NL63, Alphacoronavirus WA2028, Avian metapneumovirus (AMPV), Betacoronavirus HKU1, Betacoronavirus HKU15, Betacoronavirus HKU33, Betacoronavirus OC43, Chikungunya virus, Crimean-Congo Hemorrhagic Fever Virus, Dengue Virus, Eastern Equine Encephalitis Virus (EEEV), Enterovirus D68 (EV-D68), Foot and Mouth Disease Virus, Hanta Virus, Hendra Virus, Hepatitis B Virus, Hepatitis C Virus, HMPV, Human Parainfluenzavirus 1 (HPIV1), Human Parainfluenzavirus 3 (HPIV3), Infectious Salmon Anemia Virus, Influenza A Virus, Influenza B Virus, Lassa Virus, Marburg Virus, Middle East Respiratory Syndrome Coronavirus (MERS-COV), Newcastle Disease Virus (NDV), Nipah Virus, Norwalk Virus, Rabies Virus, Respiratory Syncytial Virus, Reston Ebola virus, Rhinovirus, Rift Valley Fever Virus, Rubella virus, SARS-COV-1, SARS-COV-2, Sudan Ebola virus, Venezuelan Equine Encephalitis Virus (VEEV), Vesicular Stomatitis Virus, Western Equine Encephalitis Virus (WEEV), Yellow Fever Virus, Zaire Ebola virus, and Zika Virus.

176. The isolated RNA polynucleotide of any one of claims 167-175, wherein the template regions are native to the virus.

177. The isolated RNA polynucleotide of any one of claims 167-175, wherein the template regions are variants of template regions native to the virus, wherein the variant has at least 90%, 91%, 92%, 93%, 94%, 95%, 96%, 97%, 98%, or 99% sequence identity to the template region native to the virus.

178. The isolated RNA polynucleotide of any one of claims 167-177, wherein each of the L and the R regions of the template regions comprise fewer than 10, 9, 8, 7, 6, 5, 4, 3, or 2 variations relative to template regions native to the virus.

179. The isolated RNA polynucleotide of any one of claims 167-178, wherein each of the L and the R regions of the template regions vary from template regions native to the virus by not more than 10, 9, 8, 7, 6, 5, 4, 3, or 2 substitutions that are not involved in 5′ capping.

180. The isolated RNA polynucleotide of any one of claims 167-179, wherein each of the L and the R regions of the template regions varies from template regions native to the virus by not more than 1 substitution that is not involved in 5′ capping.

181. The isolated RNA polynucleotide of any one of claims 167-180, wherein the template regions are nucleoside modified, wherein the percentage of modified nucleosides is not more than 40%, 35%, 30%, 25%, 20%, 15%, 10%, or 5%.

182. The isolated RNA polynucleotide of any one of claims 167-181, wherein the template regions are nucleoside modified, wherein the percentage of modified nucleosides at least 30%, 35%, 40%, 45%, 50%, 55%, 60%, 65%, 70%, 75%, 80%, 90%, 95%, or 100%.

183. The isolated RNA polynucleotide of any one of claims 167-182, wherein the nucleoside modification is a nonimmunogenic uridine modification, and the percentage of modified uridine modifications is more than 30%, 35%, 40%, 45%, 50%, 55%, 60%, 65%, 70%, 75%, 80%, 90%, or 95%, or is 100%.

184. The isolated RNA polynucleotide of any one of claims 167-183, wherein the nucleoside modification is a nonimmunogenic cytidine modification, and the percentage of modified cytidine modifications is more than 30%, 35%, 40%, 45%, 50%, 55%, 60%, 65%, 70%, 75%, 80%, 90%, or 95%, or is 100%.

185. The isolated RNA polynucleotide of any one of claims 167-184, wherein the nucleoside modification is a nonimmunogenic adenosine modification, and the percentage of modified adenosine modifications is about 1%, 5%, 10%, 15%, 20%, 25%, or 30%.

186. The isolated RNA polynucleotide of any one of claims 167-185, wherein the isolated

187. The isolated RNA polynucleotide of any one of claims 167-186, wherein the 5′ end of the L region comprises a 5′ cap structure.

188. The isolated RNA polynucleotide of any one of claims 167-187, wherein the 5′ end of the L region comprises one or more variations associated with a 5′ cap structure.

189. The isolated RNA polynucleotide of any one of claims 186-188, wherein the 5′-cap structure is selected from the group consisting of Cap 0, Cap 0 (3′-O-Me), Cap 1, Cap 1 (3′-O-Me), Cap 2, Cap 2 (3′-O-Me), Anti-Reverse Cap Analog (ARCA), inosine, N1-methyl-guanosine, 2′-fluoro-guanosine, 7-deaza-guanosine, 8-oxo-guanosine, 2-amino-guanosine, locked nucleic acid guanosine (LNA-guanosine), and 2-azido-guanosine structure.

190. The isolated RNA polynucleotide of any one of claims 167-185, wherein the isolated polynucleotide does not comprise a 5′ cap structure (uncapped).

191. The isolated RNA polynucleotide of any one of claims 167-190, wherein the 5′ end of the L region does not comprise a 5′ cap structure (uncapped).

192. The isolated RNA polynucleotide of claim 190 or 191, wherein the 5′ end of the isolated polynucleotide comprises a 5′-monophosphate, 5′-diphosphate, or 5′-triphosphate.

193. The isolated RNA polynucleotide of claim 190 or 191, wherein the 5′ end of the isolated polynucleotide does not comprise a 5′-phosphate (dephosphorylated).

194. The isolated RNA polynucleotide of claim 172, wherein the template regions are the reverse complement of template regions native to the virus.

195. The isolated RNA polynucleotide of claim 172 or 194, wherein the template regions are variants of a reverse complement of template regions native to the virus, wherein the variants have at least 90%, 91%, 92%, 93%, 94%, 95%, 96%, 97%, 98%, or 99% sequence identity to the reverse complement of the template regions native to the virus.

196. The isolated RNA polynucleotide of claim 172, 194, or 195, wherein the reverse complements of each of the L and the R regions vary from the reverse complements of template regions native to the virus by not more than 10, 9, 8, 7, 6, 5, 4, 3, or 2 substitutions that are not involved in 5′ capping.

197. The isolated RNA polynucleotide of any one of claim 172 or 194-196, wherein the reverse complements of each of the L and the R regions vary from the reverse complements of template regions native to the virus by not more than 1 substitution that is not involved in 5′ capping.

198. The isolated RNA polynucleotide of any one of claim 172 or 194-196, wherein the 5′ end of the reverse complement of the R region encodes a 5′ cap structure.

199. The isolated RNA polynucleotide of any one of claim 172 or 194-198, wherein the 5′ end of the R region is capped.

200. The isolated RNA polynucleotide of any one of claims 167-199, wherein the coding sequence encodes more than one polypeptide, which are separated by one or more ribosomal skipping sequence.

201. The isolated RNA polynucleotide of any one of claims 167-200, wherein the coding region further comprises one or more regulatory elements selected from the group consisting of ribosomal binding site, Kozak sequence, Shine-Dalgarno sequence, ribozyme, riboswitch, promoter, microRNA binding site, and internal ribosomal entry site (IRES).

202. The isolated RNA polynucleotide of claim 201, wherein the one or more regulatory elements are operably linked to the coding sequence.

203. The isolated RNA polynucleotide of any one of claims 167-202, further comprising a polyadenylation signal and/or a 3′ poly(A) tail.

204. The isolated RNA polynucleotide of any one of claims 167-203, wherein the RNA-dependent polymerase is an RNA-dependent RNA polymerase.

205. The isolated RNA polynucleotide of any one of claims 167-203, wherein the RNA-dependent polymerase is an RNA-dependent DNA polymerase.

206. The isolated RNA polynucleotide of any one of claims 167-205, wherein the RNA-dependent polymerase is a polymerase is from the virus.

207. The isolated RNA polynucleotide of any one of claim 167-206, wherein the isolated RNA polynucleotide is a single stranded RNA.

208. The isolated RNA polynucleotide of any one of claims 167-207, wherein the isolated polynucleotide is in linear form.

209. The isolated RNA polynucleotide of any one of claims 167-208, wherein the isolated polynucleotide is in a covalently-closed circular form.

210. An isolated DNA polynucleotide encoding the isolated RNA polynucleotide of any one of claims 1-209.

211. A cell or cell line comprising the isolated DNA polynucleotide of claim 210.

212. A vector comprising the isolated RNA polynucleotide of any one of claims 1-209 or the isolated DNA polynucleotide of claim 210.

213. The vector of claim 212, wherein the vector is a viral vector or an expression vector.

214. The vector of claim 213, wherein the viral vector is selected from the group consisting of adenovirus vector, adeno-associated virus vector, poxvirus vector, retrovirus vector, lentivirus vector, herpesvirus vector, alphavirus vector, and baculovirus vector. s

215. An RNA-protein complex, comprising
the isolated RNA polynucleotide of any one of claims 1-209, and
an RNA-binding protein;
wherein the isolated RNA polynucleotide of the RNA-protein complex has increased stability as compared to the isolated RNA polypeptide without the RNA binding protein.

216. The RNA-protein complex of claim 215, wherein the RNA-binding protein is a viral nucleocapsid protein (N) or viral capsid protein.

217. The RNA-protein complex of claim 215 or 216, wherein the RNA-binding protein is a viral nucleocapsid protein (N) or a viral capsid protein of the virus.

218. The RNA-protein complex of claim 216 or 217, wherein the viral nucleocapsid protein or a viral capsid protein from an influenza virus, sarbecovirus, pneumovirus, paramyxovirus, henipavirus, or hepadnavirus.

219. A composition comprising the isolated RNA polynucleotide of any one of claims 1-209, the isolated DNA polynucleotide of claim 210, the cell or cell line of claim 211, the vector of any one of claims 212-214, or the RNA-protein complex of any one of claims 215-218.

220. The composition of claim 219, further comprising a pharmaceutically acceptable carrier.

221. A nanoparticle comprising the isolated RNA polynucleotide of any one of claims 1-209, the isolated DNA polynucleotide of claim 210, or the RNA-protein complex of any one of claims 215-218.

222. A method, comprising
administering to a subject in need thereof a therapeutically effective amount of the isolated RNA polynucleotide of any one of claims 1-209, the isolated DNA polynucleotide of claim 210, the cell or cell line of claim 211, the vector of any one of claims 212-214, the RNA-protein complex of any one of claims 215-218, the composition of claim 219 or 220, or the nanoparticle of claim 221.

223. The method of claim 222, further comprising administering to a subject in need thereof a therapeutically effective amount of a second isolated RNA polynucleotide of any one of claims 1-209, a second isolated DNA polynucleotide of claim 210, a second cell or cell line of claim 211, a second vector of any one of claims 212-214, a second RNA-protein complex of any one of claims 215-218, a second composition of claim 219 or 220, or a second nanoparticle of claim 221.

224. The method of claim 222 or 223, wherein the subject is a human, cow, pig, sheep, horse, deer, rumenants, rodent, fish, or fowl.

225. The method of any one of claims 222-224, wherein the subject has a disease or disorder resulting from a viral infection.

226. The method of any one of claims 222-225, wherein the subject has an infection with a virus.

227. The method of any one of claims 222-226, wherein the administration is by intratracheal or inhalation, intranasal, oral, rectal, vaginal, transmucosal, or intestinal administration; parenteral delivery, including intradermal, transdermal (topical), intramuscular, subcutaneous, intramedullary injections, as well as intrathecal, direct intraventricular, intravenous, or intraperitoneal administration.

228. A method, comprising
contacting a cell with the isolated RNA polynucleotide of any one of claims 1-209, the isolated DNA polynucleotide of claim 210, the vector of any one of claims 212-214, the RNA-protein complex of any one of claims 215-218, or the composition of claim 219 or 220, or the nanoparticle of claim 221.

229. The method of claim 228, wherein the contacting is in vitro or ex vivo.

230. A method, comprising
administering to a subject in need thereof
(i) a therapeutically effective amount of the isolated RNA polynucleotide of any one of claims 1-209, the isolated DNA polynucleotide of claim 210, the cell or cell line of claim 211, the vector of any one of claims 212-214, the RNA-protein complex of any one of claims 215-218, or the composition of claim 219 or 220, or the nanoparticle of claim 221; and
(ii) a second polynucleotide encoding a polymerase capable of interacting with and initiating the transcription or translation of the therapeutic polypeptide or polypeptide.

231. The method of claim 230, further comprising administering to the subject (iii) one or more accessory proteins associated with polymerase activity.

232. The method of claim 231, wherein the accessory protein is a nucleocapsid protein.

233. The method of claim 231 or 232, wherein the polymerase and/or accessory proteins are administered in the form of one or more nucleic acid encoding the polymerase and/or accessory proteins.

234. The method of any one of claims 230-233, wherein (i) and (ii) are administered sequentially or simultaneously.

235. The method of any one of claims 230-234, wherein (i) and (ii) are present on the same polynucleotide.

236. The method of any one of claims 230-234, wherein (i) and (ii) are present on separate polynucleotides.

237. The method of any one of claims 230-236, further comprising administering to a subject in need thereof a therapeutically effective amount of a second isolated RNA polynucleotide of any one of claims 1-209, a second isolated DNA polynucleotide of claim 210, a second cell or cell line of claim 211, a second vector of any one of claims 212-214, a second RNA-protein complex of any one of claims 215-218, a second composition of claim 219 or 220, or a second nanoparticle of claim 221.

238. The method of any one of claims 230-237, wherein the subject is a human, cow, pig, sheep, horse, deer, rumenants, rodent, fish, or fowl.

239. A method, comprising
(a) providing a DNA vector encoding the isolated RNA polynucleotide of any one of claims 1-209;
(b) linearizing the DNA vector to produce a linear DNA vector; and
(c) contacting the linear DNA vector with a RNA polymerase, thereby producing the isolated RNA polynucleotide.

240. The method of claim 239, further comprising (d) subjecting the isolated RNA polynucleotide of (c) to one or more purification steps.

241. The method of claim 240, wherein the one or more purification steps of (d) are selected from contacting the isolated RNA polynucleotide with DNAse under conditions suitable for the digestion of the DNA vector; and tangential flow filtration.

242. The method of any one of claims 239-241, wherein the DNA vector comprises a promoter capable of directing activity of the RNA polymerase and/or a restriction endonuclease recognition site.

243. The method of claim 242, wherein the RNA polymerase is a T7 RNA polymerase and the promoter is a T7 promoter.

244. The method of any one of claims 239-243, wherein linearizing the DNA vector comprises contacting the DNA vector with a restriction endonuclease that recognizes the restriction endonuclease recognition site.

245. The method of any one of claims 239-244, wherein the contacting of (c) is performed at about 50° C.

246. The method of any one of claims 239-245, wherein the contacting of (c) is performed in the presence of one or more additional factors selected from the group consisting of ribonucleotide triphosphates, modified nucleotide triphosphates, a cap analog, inorganic pyrophosphatase, and a RNAse inhibitor.

247. The method of any one of claims 239-246, further comprising formulating the isolated RNA polynucleotide into a nanoparticle.

248. A method of generating a transgenic animal or plant comprising inserting the isolated RNA polynucleotide of any one of claims 1-209, the isolated DNA polynucleotide of claim 210, the cell or cell line of claim 211, the vector of any one of claims 212-214, the RNA-protein complex of any one of claims 215-218, or the composition of claim 219 or 220, or the nanoparticle of claim 221 into an animal or plant, thereby generating a transgenic animal or plant.

249. The method of claim 248, wherein the coding sequence of the encodes an antiviral polypeptide.

250. The transgenic animal or plant of claim 248 or 249, wherein the transgenic animal or plant has increased resistance to viral infection.

251. The transgenic animal or plant of any one of claims 248-205, which is an avian, pig, fish, cow, horse, camel, dog, cat, mouse, rat, cotton rat, hamster, ferret, primate, or other commercially valuable animal or plant species.
